(19) United States
(12) Patent Application Publication
(10) Pub. No.: US 2026/0226167 A1
(43) Pub. Date: Aug. 6, 2026

(54) IN VIVO T CELL GENE EDITING

(71) Applicant: Genzyme Corporation, Cambridge, MA (US)

(72) Inventors: Christopher BORGES, Reading, MA (US); Viktor T. LEMGART, Boston, MA (US); Matthew MACKEY, Melrose, MA (US); Shu-Wei WU, Somerville, MA (US)

(21) Appl. No.: 19/530,031

(22) Filed: Feb. 4, 2026

Related U.S. Application Data
(60) Provisional application No. 63/753,894, filed on Feb. 4, 2025.
(60) Provisional application No. 63/796,975, filed on Apr. 29, 2025.

(51) Int. Cl. C07K 16/28; A61K 39/395; A61K 48/00; C07K 14/705; C07K 14/725; C12N 15/88

(52) U.S. Cl. CPC C07K 16/2803; A61K 39/3955; A61K 48/0033; A61K 48/005; C07K 14/7051; C07K 14/70517; C07K 14/70521; C12N 15/88; C07K 2317/52; C07K 2317/53; C07K 2317/55; C07K 2317/569; C07K 2317/92; C07K 2319/02; C07K 2319/03

(57) ABSTRACT

The present disclosure relates generally to compositions and methods for in vivo gene therapy, and more specifically to delivering mRNA encoding anti-CD19 chimeric antigen receptors (CARs) to immune cells (e.g., T cells, such as CD8+ T cells) in vivo.

### CROSS REFERENCE TO RELATED APPLICATIONS

This application claims priority to U.S. Provisional Application No. 63/753,894, filed Feb. 4, 2025, and U.S. Provisional Application No. 63/796,975, filed Apr. 29, 2025, the contents of each which are incorporated herein by reference in their entireties.

### REFERENCE TO AN ELECTRONIC SEQUENCE LISTING

The content of the electronic sequence listing (159792020800seqlist.xml; Size: 786,216 bytes; and Date of Creation: Feb. 3, 2026) is herein incorporated by reference in its entirety.

### FIELD

The present disclosure relates generally to compositions and methods for in vivo gene therapy, and more specifically to delivering mRNA-based therapeutics to immune cells in vivo.

### BACKGROUND

Genetic modification of T cells with chimeric antigen receptors (CARs) to target specific diseases has shown impressive clinical responses in patients with hematologic malignancies. However, several barriers remain before this therapy is available to a broader patient population. Currently, CAR-T cell therapy production is carried out ex vivo, including genetic modification of the patient's T cells in culture before infusing the cells back into the patient. The ex vivo methods required to generate sufficient numbers of tumor-specific T cells are complex thereby hindering widespread application to treat cancer patients. Finally, CAR T cell therapy results in acute and chronic toxicities that limit its overall therapeutic index in cancer patients [5]. The onset of immune activation, known as cytokine release syndrome (CRS), is the most prevalent adverse effect following CAR T cell infusion. In order to overcome these significant challenges and limitations, more innovative strategies are required to program T cells to express CARs.

In-vitro transcribed (IVT) mRNA has, in recent years, proven an effective technology to express therapeutic proteins and antigens in cells, making it a promising alternative to DNA-based products. By utilizing IVT mRNA, researchers allow for transient protein expression without causing integration into the genome, and do not require nuclear import or slow cytoplasmic diffusion. However, to function in vivo, effective and stable delivery platforms, protecting the mRNA from degradation and allowing for specific target cell uptake, are required. One such delivery system that has entered the clinic and proven successful is lipid nanoparticles (LNPs), employing an ionizable cationic lipid to condense nucleic acids into a relatively uniform solid lipid nanoparticle approximating 100 nm in diameter. Remarkably, the recent authorization of two coronavirus (COVID-19) vaccines utilizing LNPs to deliver mRNA intramuscularly stands out as noticeable examples. However, to date, the use and design of LNPs for systemic delivery have primarily allowed for cellular uptake by hepatocytes and Kupffer cells of the liver. Delivery of a nuclei acid payload toward a cell type of interest may be achieved with the addition of targeting ligands.

Thus, what is needed is a delivery platform that overcomes some of the barriers in existing T cell therapies. More particularly, what is needed is a delivery platform that overcomes some of the barriers in existing CAR T cell therapies for B cell hematologic malignancies.

### BRIEF SUMMARY

The present disclosure in one aspect provides a polypeptide comprising an anti-CD19 chimeric antigen receptor (anti-CD19 CAR), wherein the anti-CD19 CAR comprises: (a) an anti-CD19 single domain antibody (sdAb or VHH) as disclosed herein; (b) a hinge domain, (c) a transmembrane domain, (d) an intracellular signaling domain, and (e) optionally a co-stimulatory domain. The present disclosure in another aspect provides anti-CD19 single domain antibodies (sdAbs or VHH antibodies) as disclosed herein. The sdABs of the disclosure can be used independently (e.g., as an immunotherapy), as a component of a bispecific a multispecific antibody, for diagnostic applications, or can be used to engineer CAR-T cells.

The present disclosure in one embodiment provides a polypeptide comprising an anti-CD19 chimeric antigen receptor (anti-CD19 CAR), wherein the anti-CD19 CAR comprises: (a) an anti-CD19 antigen-binding domain, wherein the anti-CD19 antigen-binding domain is an anti-CD19 single domain antibody (anti-CD19 sdAb) domain comprising an anti-CD19 VHH comprising: i) an H-CDR1 comprising the amino acid sequence (Kabat numbering) of SEQ ID NO:427, an H-CDR2 comprising the amino acid sequence of SEQ ID NO: 428 (Kabat numbering), and an H-CDR3 comprising the amino acid sequence of SEQ ID NO: 429 (Kabat numbering), or (ii) an H-CDR1 comprising the amino acid sequence of SEQ ID NO: 475 (Kabat numbering), an H-CDR2 comprising the amino acid sequence of SEQ ID NO: 476 (Kabat numbering), and an H-CDR3 comprising the amino acid sequence of SEQ ID NO: 477 (Kabat numbering); (b) a hinge domain, (c) a transmembrane domain, (d) an intracellular signaling domain, and (e) optionally a co-stimulatory domain.

In some embodiments according to any of the polypeptides described above, the anti-CD19 sdAb comprises a VHH comprising the amino acid sequence of SEQ ID NO: 433 or 481, or an amino acid sequence comprising at least about 80% sequence identity to the amino acid sequence of SEQ ID NO: 433 or 481, or an amino acid sequence comprising at least about 85% sequence identity to the amino acid sequence of SEQ ID NO: 433 or SEQ ID NO: 481, or an amino acid sequence comprising at least about 90% sequence identity to the amino acid sequence of SEQ ID NO: 433 or SEQ ID NO: 481, or an amino acid sequence comprising at least about 95% sequence identity to the amino acid sequence of SEQ ID NO: 433 or SEQ ID NO: 481, or an amino acid sequence comprising at least about 98% sequence identity to the amino acid sequence of SEQ ID NO: 433 or SEQ ID NO: 481, or an amino acid sequence comprising at least about 99% sequence identity to the amino acid sequence of SEQ ID NO: 433 or SEQ ID NO: 481.

The present disclosure in one embodiment provides a polypeptide comprising an anti-CD19 chimeric antigen receptor (anti-CD19 CAR), wherein the anti-CD19 CAR comprises: (a) an anti-CD19 antigen-binding domain, wherein the anti-CD19 antigen-binding domain is an anti-CD19 single domain antibody (anti-CD19 sdAb) domain comprising an anti-CD19 VHH comprising: i) an H-CDR1 comprising the amino acid sequence (Kabat numbering) of SEQ ID NO: 740, an H-CDR2 comprising the amino acid sequence of SEQ ID NO: 741 (Kabat numbering), and an H-CDR3 comprising the amino acid sequence of SEQ ID NO: 743 (Kabat numbering), or (ii) an H-CDR1 comprising the amino acid sequence of SEQ ID NO: 740 (Kabat numbering), an H-CDR2 comprising the amino acid sequence of SEQ ID NO: 741 (Kabat numbering), and an H-CDR3 comprising the amino acid sequence of SEQ ID NO: 744 (Kabat numbering); (b) a hinge domain, (c) a transmembrane domain, (d) an intracellular signaling domain, and (e) optionally a co-stimulatory domain.

In some embodiments according to any of the polypeptides described above, the anti-CD19 sdAb comprises a VHH comprising the amino acid sequence of SEQ ID NO: 699, SEQ ID NO: 739 or SEQ ID NO: 721, or an amino acid sequence comprising at least about 80% sequence identity to the amino acid sequence of SEQ ID NO: 699, SEQ ID NO: 739 or SEQ ID NO: 721, or an amino acid sequence comprising at least about 85% sequence identity to the amino acid sequence of SEQ ID NO: 699, SEQ ID NO: 739 or SEQ ID NO: 721, or an amino acid sequence comprising at least about 90% sequence identity to the amino acid sequence of SEQ ID NO: 699, SEQ ID NO: 739 or SEQ ID NO: 721, or an amino acid sequence comprising at least about 95% sequence identity to the amino acid sequence of SEQ ID NO: 699, SEQ ID NO: 739 or SEQ ID NO: 721, or an amino acid sequence comprising at least about 98% sequence identity to the amino acid sequence of SEQ ID NO: 699, SEQ ID NO: 739 or SEQ ID NO: 721, or an amino acid sequence comprising at least about 99% sequence identity to the amino acid sequence of SEQ ID NO: 699, SEQ ID NO: 739 or SEQ ID NO: 721.

The present disclosure in another embodiment provides a polypeptide comprising an anti-CD19 chimeric antigen receptor (anti-CD19 CAR), wherein the anti-CD19 CAR comprises: (a) an anti-CD19 antigen-binding domain, wherein the anti-CD19 antigen-binding domain is an anti-CD19 single domain antibody (anti-CD19 sdAb) domain comprising an anti-CD19 VHH comprising: an H-CDR1 comprising the amino acid sequence (Kabat numbering) of SEQ ID NO: 850, an H-CDR2 comprising the amino acid sequence of SEQ ID NO: 851 (Kabat numbering), and an H-CDR3 comprising the amino acid sequence of SEQ ID NO: 852 (Kabat numbering); (b) a hinge domain, (c) a transmembrane domain, (d) an intracellular signaling domain, and (e) optionally a co-stimulatory domain.

In some embodiments according to any of the polypeptides described above, the anti-CD19 sdAb comprises a VHH comprising the amino acid sequence of SEQ ID NO: 711 or SEQ ID NO: 712, or an amino acid sequence comprising at least about 80% sequence identity to the amino acid sequence of SEQ ID NO: 711 or SEQ ID NO: 712, or an amino acid sequence comprising at least about 85% sequence identity to the amino acid sequence of SEQ ID NO: 711 or SEQ ID NO: 712, or an amino acid sequence comprising at least about 90% sequence identity to the amino acid sequence of SEQ ID NO: 711 or SEQ ID NO: 712, or an amino acid sequence comprising at least about 95% sequence identity to the amino acid sequence of SEQ ID NO: 711 or SEQ ID NO: 712, or an amino acid sequence comprising at least about 98% sequence identity to the amino acid sequence of SEQ ID NO: 711 or SEQ ID NO: 712, or an amino acid sequence comprising at least about 99% sequence identity to the amino acid sequence of SEQ ID NO: 711 or SEQ ID NO: 712.

In some embodiments according to any of the polypeptides described above, the hinge domain is derived from a protein selected from the group consisting of CD28, CD5, CD9, CD2, CD27, CD28, IgG, IgK, and CD8α. In some embodiments, the hinge domain is derived from CD28 or CD8α.

In some embodiments according to any of the polypeptides described above, the transmembrane domain is derived from a protein selected from the group consisting of CD28, CD3ε, CD45, CD4, CD5, CD8α, CD9, CD16, CD22, CD33, CD37, CD64, CD80, CD86, CD147, 4-1BB (CD137), CD152, PD-1, CD2, CD27, CD11b, CD11d, CD11a, CD11c, CD84, and CD86. In some embodiments, the transmembrane domain is derived from CD28 or CD8α.

In some embodiments according to any of the polypeptides described above, the hinge domain and the transmembrane domain are derived from the same protein. In some embodiments, the hinge domain and the transmembrane domain are derived from CD28 and comprises the amino acid sequence of SEQ ID NO: 431 or 434.

In some embodiments according to any of the polypeptides described above, the intracellular signaling domain is derived from a protein selected from the group consisting of CD3ζ, FcεRIβ, FcεRIγ, FcRβ, CD3γ, CD3δ, CD3ε, CD5, CD22, CD79a, CD79b, and CD66d. In some embodiments, the intracellular signaling domain is derived from CD3ζ. In some embodiments, the intracellular signaling domain comprises the amino acid sequence of SEQ ID NO: 544.

In some embodiments according to any of the polypeptides described above, the anti-CD19 CAR further comprises a co-stimulatory domain derived from a protein selected from the group consisting of CD27, CD28, CD137 (4-1BB), OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR, LGALS9, CD83, ligands of CD83, and any combination thereof. In some embodiments, the co-stimulatory domain is derived from CD137 (4-1BB) or CD28. In some embodiments, the co-stimulatory domain comprises the amino acid sequence of SEQ ID NO: 432 or 543.

In some embodiments according to any of the polypeptides described above, the anti-CD19 CAR further comprises a signal peptide sequence that comprises a signal peptide, wherein the lead peptide sequence is connected to the N-terminus of the anti-CD19 CAR. In some embodiments, the signal peptide sequence is derived from a CD8a propeptide. In some embodiments, the signal peptide sequence comprises the amino acid sequence of SEQ ID NO: 515.

In some embodiments according to any of the polypeptides described above, the C-terminus of the anti-CD19 sdAb and the N-terminus of the hinge domain are connected by a peptide linker. In some embodiments, the peptide linker is AS or AAA.

In some embodiments according to any of the polypeptides described above, the anti-CD19 CAR comprises the amino acid sequence of any one of SEQ ID NOs: 49-51 and 435-437.

In some embodiments according to any of the polypeptides described above, the anti-CD19 CAR comprises the amino acid sequence of SEQ ID NO: 49.

In some embodiments according to any of the polypeptides described above, the anti-CD19 CAR comprises the amino acid sequence of SEQ ID NO: 50.

In some embodiments according to any of the polypeptides described above, the anti-CD19 CAR comprises the amino acid sequence of SEQ ID NO: 51.

In some embodiments according to any of the polypeptides described above, the anti-CD19 CAR comprises the amino acid sequence of SEQ ID NO: 435.

In some embodiments according to any of the polypeptides described above, the anti-CD19 CAR comprises the amino acid sequence of SEQ ID NO: 436.

In some embodiments according to any of the polypeptides described above, the anti-CD19 CAR comprises the amino acid sequence of SEQ ID NO: 437.

In some embodiments according to any of the polypeptides described above, the anti-CD19 CAR comprises the amino acid sequence of any one of SEQ ID NOs: 576-623. In some embodiments according to any of the polypeptides described above, the anti-CD19 CAR comprises the amino acid sequence of any one of SEQ ID NOs: 624-681.

In some embodiments according to any of the polypeptides described above, the anti-CD19 CAR comprises the amino acid sequence of SEQ ID NO: 584.

In some embodiments according to any of the polypeptides described above, the anti-CD19 CAR comprises the amino acid sequence of SEQ ID NO: 632.

In some embodiments according to any of the polypeptides described above, the anti-CD19 CAR comprises the amino acid sequence of SEQ ID NO: 585.

In some embodiments according to any of the polypeptides described above, the anti-CD19 CAR comprises the amino acid sequence of SEQ ID NO: 633.

In some embodiments according to any of the polypeptides described above, the anti-CD19 CAR comprises the amino acid sequence of SEQ ID NO: 621.

In some embodiments according to any of the polypeptides described above, the anti-CD19 CAR comprises the amino acid sequence of SEQ ID NO: 675.

In some embodiments according to any of the polypeptides described above, the anti-CD19 CAR comprises the amino acid sequence of SEQ ID NO: 591.

In some embodiments according to any of the polypeptides described above, the anti-CD19 CAR comprises the amino acid sequence of SEQ ID NO: 601.

In some embodiments according to any of the polypeptides described above, the anti-CD19 CAR comprises the amino acid sequence of SEQ ID NO: 602.

In some embodiments according to any of the polypeptides described above, the anti-CD19 CAR comprises the amino acid sequence of SEQ ID NO: 639.

In some embodiments according to any of the polypeptides described above, the anti-CD19 CAR comprises the amino acid sequence of SEQ ID NO: 649.

In some embodiments according to any of the polypeptides described above, the anti-CD19 CAR comprises the amino acid sequence of SEQ ID NO: 650.

In another aspect, there is provided an isolated nucleic acid comprising a nucleic acid sequence selected from the group consisting of SEQ ID NO:46-48, 114, 145, 441-458, 482-511, and 570-575.

Also provided herein are isolated nucleic acids encoding any of the polypeptides described above, vectors comprising any of the isolated nucleic acids described herein, and cells comprising any of the vectors or isolated nucleic acids described herein or any of the polypeptides described above. In some embodiments, the isolated nucleic acid comprises or is RNA. In some embodiments, the RNA comprises or is mRNA. In some embodiments, the RNA comprises or is any one of SEQ ID NOs:570-575. In some embodiments, the cell is an immune cell. In some embodiments, the immune cell is a T cell. In some embodiments, the cell is a human cell.

In another aspect, there is provided a lipid nanoparticle (LNP) comprising: (a) a lipid-immune cell targeting group conjugate comprising the compound of Formula (II): [Lipid]-[optional linker]-[binding domain], wherein the binding domain specifically binds to human CD8α, (b) an ionizable cationic lipid, and (c) a nucleic acid encoding any of the polypeptides described above, wherein the nucleic acid is encapsulated in the LNP.

In another aspect, there is provided a lipid nanoparticle (LNP) comprising: (a) a lipid-immune cell targeting group conjugate comprising the compound of Formula (II): [Lipid]-[optional linker]-[binding domain], wherein the binding domain specifically binds to human CD8α, (b) an ionizable cationic lipid, and (c) a nucleic acid comprising a nucleic acid sequence selected from the group consisting of SEQ ID NO:46-48, 114, 145, 441-458, 482-511, and 570-575, wherein the nucleic acid is encapsulated in the LNP.

In some embodiments according to any of the LNPs described above, the binding domain is an immunoglobulin single variable domain (ISVD) that specifically binds to human CD8α, wherein the ISVD comprises a complementarity-determining region 1 (CDR1), a CDR2, and a CDR3 of an ISVD having the sequence selected from the group consisting of SEQ ID NOs: 160-179; or the binding domain is an immunoglobulin single variable domain (ISVD) that specifically binds to human CD8α, wherein the ISVD specifically binding to CD8α comprises a CDR1, a CDR2, and a CDR3 according to the Abm CDR definition, wherein CDR1 is chosen from the group consisting of: (i) SEQ ID NO: 244; and (ii) amino acid sequences that have 3, 2, or 1 amino acid difference with at least one of the amino acid sequences of SEQ ID NO: 244; wherein CDR2 is chosen from the group consisting of: (i) SEQ ID NO: 246; and (ii) amino acid sequences that have 3, 2, or 1 amino acid difference with at least one of the amino acid sequences of SEQ ID NO: 246; and wherein CDR3 is chosen from the group consisting of: (i) SEQ ID NO: 248; and (ii) amino acid sequences that have 3, 2, or 1 amino acid difference with at least one of the amino acid sequences of SEQ ID NO: 248.

In some embodiments according to any of the LNPs described above, the binding domain specifically binding to human CD8α is covalently coupled to the Lipid in Formula (II) via a linker comprising polyethylene glycol (PEG). In some embodiments, the Lipid in Formula (II) covalently coupled to the binding domain is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the Lipid in Formula (II) covalently coupled to the binding domain is DSPE. In some embodiments, the PEG is PEG 3400 (PEG 3.4K).

In some embodiments according to any of the LNPs described above, the immunoglobulin single variable domain comprises SEQ ID NO: 9, SEQ ID NO: 169, or SEQ ID NO: 44.

In some embodiments according to any of the LNPs described above, the LNP further comprises a structural lipid, a neutral phospholipid, or a free PEG-lipid, or any combination thereof. In some embodiments, the structural lipid comprises or is sterol. In some embodiments, the sterol comprises or is cholesterol.

In some embodiments according to any of the LNPs described above, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin. In some embodiments, the neutral phospholipid comprises or is DSPC.

In some embodiments according to any of the LNPs described above, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. In some embodiments, the free PEG lipid is PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, diacylphosphatidylethanolamine comprising Dipalmitoyl (C16) chain or Distearoyl (C18) chain, or a PEG-distearoyl-phosphatidylethanolamine (PEG-DSPE) lipid. In some embodiments, the PEG-DAG comprises PEG-DMG, PEG-DPG, or PEG-DSG, or any combination thereof. In some embodiments, the free PEG-lipid comprises PEG-DPG. In some embodiments, the PEG-DPG comprises or is PEG 2000-DPG (DPG-PEG 2000).

In some embodiments according to any of the LNPs described above, the nucleic acid comprises or is RNA. In some embodiments, the RNA comprises or is mRNA. In some embodiments, the RNA comprises or is any one of SEQ ID NOs:570-575. In some embodiments, the mRNA comprises a 5′ Cap, a 5′ untranslated region (UTR), a sequence encoding a polypeptide, a 3′ UTR, and optionally a poly A tail. In some embodiments, the nucleic acid comprises: (1) optionally, a 5′ cap; (2) optionally, a 5′ UTR region; (3) nucleotides encoding any of the polypeptides described above; (4) optionally, a 3′ UTR region; and (5) optionally, a polyA tail. In some embodiments, the polypeptide encoded by the nucleic acid comprises the following formula, arranged from N-terminus to C-terminus: [Signal peptide sequence (optional)]-[anti-CD19 sdAb]-[Peptide Linker (optional)]-[Hinge domain]-[Transmembrane domain]-[Co-stimulatory domain]-[Intracellular signaling domain]. In some embodiments, the nucleic acid comprises pseudouridine. In some embodiments, the pseudouridine is N1-methyl-pseudouridine.

In some embodiments according to any of the LNPs described above, the ionizable cationic lipid comprises a compound of Formula (I):

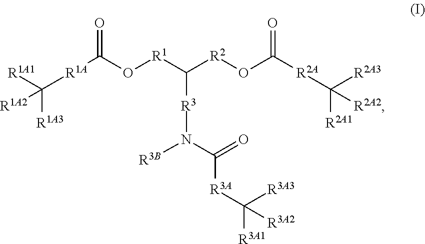

or a salt thereof, or both, wherein: R¹, R², and R³ are each independently a bond or C₁₋₃ alkylene; R^{1A}, R^{2A}, and R^{3A} are each independently a bond or C₁₋₁₀ alkylene; R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}; R^{a1} and R^{a2} are each independently C₁₋₂₀ alkyl or C₁₋₂₀ alkenyl; R^{3B} is

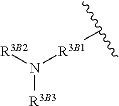

R^{3B1} is C₁₋₆ alkylene; and R^{3B2} and R^{3B3} are each independently H, unsubstituted C₁₋₆ alkyl, or C₁₋₆ alkyl substituted with 1 or 2-OH.

In some embodiments according to any of the LNPs described above, R¹, R², and R³ are each independently a bond or methylene; R^{1A} and R^{2A} are each C₁₋₁₀ alkylene; R^{3A} is C₁₋₅ alkylene; R^{1A1}, R^{1A2}, R^{2A1}, R^{2A2}, R^{3A1}, and R^{3A2} are each H; R^{1A3} and R^{2A3} are each C₁₋₂₀ alkenyl; R^{3A3} is —C(O)O(C₁₋₂₀ alkyl); R^{3B} is

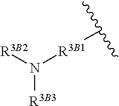

R^{3B1} is C₂₋₄ alkylene; and R^{3B2} and R^{3B3} are each methyl. In some embodiments, R^{3B1} is —(CH₂)₃—.

In some embodiments according to any of the LNPs described above, the ionizable cationic lipid comprises

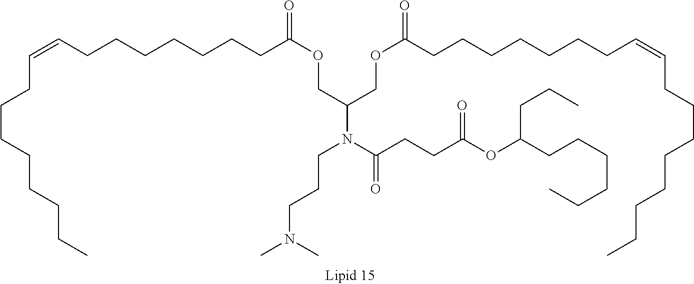 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

or a salt thereof, or both.

In some embodiments according to any of the LNPs described above, the cationic lipid has a concentration between about 10 mol % and about 60 mol % of the LNP. In some embodiments, the LNP comprises cholesterol at a concentration between about 25 mol % and about 45 mol % of the LNP. In some embodiments, the LNP comprises DSPC at a concentration between about 5 mol % and about 25% mol % of the LNP. In some embodiments, the LNP comprises DPG-PEG2K at a concentration between about 0.5 mol % and about 2.5 mol % of the LNP. In some embodiments, the LNP comprises cationic lipid at a concentration between about 49 mol % and about 50 mol % of the LNP, such as about 49.1, 49.2, 49.3, 49.4, 49.5, 49.6, 49.7, 49.8, or 49.9 mol %. In some embodiments, the LNP comprises cholesterol at a concentration between about 25 mol % and about 30 mol % of the LNP, such as about 25, 26, 27, 28, 29, or 30 mol %. In some embodiments, the LNP comprises DSPC at a concentration between about 9 mol % and about 21 mol % of the LNP. In some embodiments, the LNP comprises DPG-PEG2K at a concentration between about 1.4 mol % and about 1.6 mol % of the LNP.

In some embodiments according to any of the LNPs described above, the LNP comprises cationic lipid at a concentration between about 10 and about 20 g per gram of mRNA in the LNP. In some embodiments, the LNP comprises cholesterol at a concentration between about 3.0 and about 5.0 g per gram of mRNA in the LNP. In some embodiments, the LNP comprises DSPC at a concentration between about 2.0 and about 5.0 g per gram of mRNA in the LNP. In some embodiments, the LNP comprises DPG-PEG2K at a concentration between about 1.0 and about 1.5 g per gram of mRNA in the LNP. In some embodiments, the LNP comprises a DSPE-PEG3.4-antibody conjugate at a concentration between about 0.05 to 0.1 g per gram of mRNA in the LNP.

In some embodiments according to any of the LNPs described above, (i) the cationic lipid has a concentration about 49.2 mol % of the LNP; (ii) the cholesterol has a concentration about 39.4 mol % of the LNP; (iii) the DSPC has a concentration about 9.8 mol % of the LNP; and (iv) the DPG-PEG2K has a concentration about 1.5% of the LNP.

In some embodiments according to any of the LNPs described above, (i) the cationic lipid has a concentration about 49.2 mol % of the LNP; (ii) the cholesterol has a concentration about 29.3 mol % of the LNP; (iii) the DSPC has a concentration about 20.0 mol % of the LNP; and (iv) the DPG-PEG2K has a concentration about 1.5% of the LNP.

In some embodiments according to any of the LNPs described above, (i) the cationic lipid has a concentration about 14.2 g/g mRNA in the LNP; (ii) the cholesterol has a concentration about 4.64 g/g mRNA in the LNP; (iii) the DSPC has a concentration about 2.37 g/g mRNA in the LNP; (iv) the DPG-PEG2K has a concentration about 1.15 g/g mRNA in the LNP; and (v) the DSPE-PEG3.4K-anti-CD8 antibody conjugate has a concentration about 0.084 g/g to 0.15 g/g mRNA in the LNP.

Also provided herein is an in vitro transcribed mRNA derived from an isolated nucleic acid encoding any of the polypeptides described above, an immune cell comprising said in vitro transcribed mRNA, and an immune cell expressing any of the polypeptides described herein. Also provided herein is a pharmaceutical composition comprising any of the polypeptides described herein, any of the isolated nucleic acids described herein, any of the vectors described herein, any of the cells of described herein, and/or any of the LNPs described herein.

In another aspect is provided a method of preparing an LNP, comprising combining any of the isolated nucleic acids described above with a mixture of lipids.

In another aspect is provided a method of delivering a nucleic acid sequence into a human cell, comprising administering any of the pharmaceutical compositions described herein. In some embodiments, the human cell is a human immune cell. In some embodiments, the human immune cell is a human T cell (such as a human CD8+ T cell). Also provided herein is a use of any of the LNPs or pharmaceutical compositions described above for the manufacture of a medicament for delivering a nucleic acid to a target cell. In some embodiments, the target cell is an immune cell (e.g., a T cell, such as a CD8+ T cell).

In another aspect is provided a method of modulating immune response in a human subject, comprising administering any of the LNPs described above, any of the isolated nucleic acids described above, any of the vectors described above, and/or any of the cells described above to the human subject, and/or expressing any of the polypeptides described above in the human subject.

In another aspect is provided a method of treating a B cell malignancy in a human subject comprising administering any of the LNPs described above, any of the isolated nucleic acids described above, any of the vectors described above, and/or any of the cells described above to the human subject, and/or expressing any of the polypeptides described above in the human subject. Also provided herein is a use of any of the polypeptides described above, any of the isolated nucleic acids described above, any of the vectors described above, any of the cells described above, any of the LNPs described above, and/or any of the pharmaceutical compositions described above for the manufacture of a medicament for the treatment of a B cell malignancy. In some embodiments, the B cell malignancy is a hematological cancer or an autoimmune disorder. In some embodiments, the B cell malignancy is a hematological cancer, wherein the cancer is hematological cancer is a B cell lymphoma. In some embodiments, the B cell lymphoma is diffuse large B cell lymphoma (DLBCL). In some embodiments, the B cell malignancy is an autoimmune disorder selected from the group consisting of rheumatoid arthritis (RA), multiple sclerosis, type 1 diabetes mellitus (TID), primary Sjögren's syndrome (pSS), and systemic lupus erythematosus (SLE).

In another aspect, the disclosure provides anti-CD19 single domain antibodies (sdAbs or VHH antibodies). In some embodiments, the sdAb has an amino acid sequence selected from the group consisting of any one of SEQ ID Nos: 684-739, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence selected from the group consisting of any one of SEQ ID Nos: 684-739.

In one embodiment, the sdAb comprises four framework regions (FR1 to FR4, respectively) and three complementarity determining regions (H-CDR1 to H-CDR3, respectively), wherein:
H-CDR1 (AbM definition) consists of an amino acid sequence selected from:
   a) the amino acid sequence of GFTFSSYAMA (SEQ ID NO: 747);
   b) amino acid sequences that have at least 80% amino acid identity with the amino acid sequence of GFTFSSYAMA (SEQ ID NO: 747); and
   c) amino acid sequences that have 3, 2, or 1 amino acid difference with the amino acid sequence of GFTFSSYAMA (SEQ ID NO: 747);
and
H-CDR2 (AbM definition) consists of an amino acid sequence selected from:
   d) the amino acid sequence of VISLSGGSVD (SEQ ID NO: 748);
   e) amino acid sequences that have at least 80% amino acid identity with the amino acid sequence of VISLSGGSVD (SEQ ID NO: 748); and
   f) amino acid sequences that have 3, 2, or 1 amino acid difference with the amino acid sequence of VISLSGGSVD (SEQ ID NO: 748);
and
H-CDR3 (AbM definition) consists of an amino acid sequence selected from:
   g) the amino acid sequence of RPVYX₁KWSERDFDY (SEQ ID NO: 838), wherein the amino acid residue X₁ is selected from M, I, L and V;
   h) amino acid sequences that have at least 80% amino acid identity with the amino acid sequence of RPVYX₁KWSERDFDY (SEQ ID NO: 838), wherein the amino acid residue X₁ is selected from M, I, L and V; and
   i) amino acid sequences that have 3, 2, or 1 amino acid difference with the amino acid sequence of RPVYX₁KWSERDFDY (SEQ ID NO: 838), wherein the amino acid residue X₁ is selected from M, I, L and V.

In some embodiments, the disclosure provides an anti-CD19 antigen-binding domain, wherein the anti-CD19 antigen-binding domain is an anti-CD19 single domain antibody (anti-CD19 sdAb) domain comprising an anti-CD19 VHH comprising: i) an H-CDR1 comprising the amino acid sequence (Abm numbering) of SEQ ID NO: 747, an H-CDR2 comprising the amino acid sequence of SEQ ID NO: 748 (Abm numbering), and an H-CDR3 comprising the amino acid sequence of SEQ ID NO: 749 (Abm numbering), or (ii) an H-CDR1 comprising the amino acid sequence of SEQ ID NO: 747 (Abm numbering), an H-CDR2 comprising the amino acid sequence of SEQ ID NO: 748 (Abm numbering), and an H-CDR3 comprising the amino acid sequence of SEQ ID NO: 750 (Abm numbering).

In another aspect, the disclosure provides anti-CD19 single domain antibodies (sdAbs or VHH antibodies). In one embodiment, the sdAb comprises four framework regions (FR1 to FR4, respectively) and three complementarity determining regions (H-CDR1 to H-CDR3, respectively), wherein:
H-CDR1 (Kabat definition) consists of an amino acid sequence selected from:
   a) the amino acid sequence of SYAMA (SEQ ID NO: 740);
   b) amino acid sequences that have at least 80% amino acid identity with the amino acid sequence of SYAMA (SEQ ID NO: 740); and
   c) amino acid sequences that have 3, 2, or 1 amino acid difference with the amino acid sequence of SYAMA (SEQ ID NO: 740);
and
H-CDR2 (Kabat definition) consists of an amino acid sequence selected from:
   d) the amino acid sequence of VISLSGGSVDYX₂DSVKG (SEQ ID NO: 839), wherein X₂ is R or A;
   e) amino acid sequences that have at least 80% amino acid identity with the amino acid sequence of VISLSGGSVDYX₂DSVKG (SEQ ID NO: 839), wherein X₂ is R or A; and
   f) amino acid sequences that have 3, 2, or 1 amino acid difference with the amino acid sequence of VISLSGGSVDYX₂DSVKG (SEQ ID NO: 839), wherein X₂ is R or A;
and
H-CDR3 (Kabat definition) consists of an amino acid sequence selected from:
   g) the amino acid sequence of RPVYX₁KWSERDFDY (SEQ ID NO: 815), wherein the amino acid residue X₁ is selected from M, I, L and V;
   h) amino acid sequences that have at least 80% amino acid identity with the amino acid sequence of RPVYX₁KWSERDFDY (SEQ ID NO: 815), wherein the amino acid residue X₁ is selected from M, I, L and V; and
   i) amino acid sequences that have 3, 2, or 1 amino acid difference with the amino acid sequence of RPVYX₁KWSERDFDY (SEQ ID NO: 815), wherein the amino acid residue X₁ is selected from M, I, L and V.

In some embodiments, the disclosure provides an anti-CD19 antigen-binding domain, wherein the anti-CD19 antigen-binding domain is an anti-CD19 single domain antibody (anti-CD19 sdAb) domain comprising an anti-CD19 VHH comprising: i) an H-CDR1 comprising the amino acid sequence (Kabat numbering) of SEQ ID NO: 740, an H-CDR2 comprising the amino acid sequence of SEQ ID NO: 741 (Kabat numbering), and an H-CDR3 comprising the amino acid sequence of SEQ ID NO: 743 (Kabat numbering), or (ii) an H-CDR1 comprising the amino acid sequence of SEQ ID NO: 740 (Kabat numbering), an H-CDR2 comprising the amino acid sequence of SEQ ID NO: 741 (Kabat numbering), and an H-CDR3 comprising the amino acid sequence of SEQ ID NO: 744 (Kabat numbering).

In some embodiments according to any of the polypeptides described above, the anti-CD19 sdAb comprises a VHH comprising the amino acid sequence of SEQ ID NO: 699, SEQ ID NO: 739 or SEQ ID NO: 721, or an amino acid sequence comprising at least about 80% sequence identity to the amino acid sequence of SEQ ID NO: 699, SEQ ID NO: 739 or SEQ ID NO: 721, or an amino acid sequence comprising at least about 85% sequence identity to the amino acid sequence of SEQ ID NO: 699, SEQ ID NO: 739 or SEQ ID NO: 721, or an amino acid sequence comprising at least about 90% sequence identity to the amino acid sequence of SEQ ID NO: 699, SEQ ID NO: 739 or SEQ ID NO: 721, or an amino acid sequence comprising at least about 95% sequence identity to the amino acid sequence of SEQ ID NO: 699, SEQ ID NO: 739 or SEQ ID NO: 721, or an amino acid sequence comprising at least about 98% sequence identity to the amino acid sequence of SEQ ID NO: 699, SEQ ID NO: 739 or SEQ ID NO: 721, or an amino acid sequence comprising at least about 99% sequence identity to the amino acid sequence of SEQ ID NO: 699, SEQ ID NO: 739 or SEQ ID NO: 721.

In some embodiments, the disclosure provides an anti-CD19 antigen-binding domain, wherein the anti-CD19 antigen-binding domain is an anti-CD19 single domain antibody (anti-CD19 sdAb) domain comprising an anti-CD19 VHH comprising: i) an H-CDR1 comprising the amino acid sequence (Kabat numbering) of SEQ ID NO: 850, an H-CDR2 comprising the amino acid sequence of SEQ ID NO: 851 (Kabat numbering), and an H-CDR3 comprising the amino acid sequence of SEQ ID NO: 852 (Kabat numbering), or (ii) an H-CDR1 comprising the amino acid sequence of SEQ ID NO: 857 (Abm numbering), an H-CDR2 comprising the amino acid sequence of SEQ ID NO: 858 (Kabat numbering), and an H-CDR3 comprising the amino acid sequence of SEQ ID NO: 859 (Kabat numbering).

### DESCRIPTION OF THE FIGURES

The patent or application file contains at least one drawing executed in color. Copies of this patent or patent application publication with color drawings will be provided by the Office upon request and payment of the necessary fee.

The present application can be understood by reference to the following description taken in conjunction with the accompanying figures.

FIG. 1 depicts proton NMR spectrum of intermediate 13-11.

FIGS. 2A-2C depict proton NMR spectra of intermediates. FIG. 2A depicts proton NMR spectrum of intermediate 13-11a; FIG. 2B depicts proton NMR spectrum of intermediate 13-11b; and FIG. 2C depicts LC-ELSD of intermediate 13-11b.

FIGS. 3A-3B depict the proton NMR spectrum and LC-CAD chromatogram of intermediate 13-10. FIG. 3A depicts proton NMR spectrum of intermediate 13-10; FIG. 3B depicts LC-CAD chromatogram of intermediate 13-10.

FIGS. 4A-1 and 4A-2 depict the proton NMR spectrum and LC-CAD chromatogram of Lipid 1. FIG. 4A-1 depicts proton NMR spectrum for Lipid 1; FIG. 4A-2 depicts the LC-CAD chromatogram of Lipid 1.

FIGS. 4B-1 and 4B-2 depict the proton NMR spectrum and LC-CAD chromatogram of Lipid 3. FIG. 4B-1 depicts proton NMR spectrum of Lipid 3; FIG. 4B-2 depicts the LC-CAD chromatogram of Lipid 3.

FIGS. 4C-1 and 4C-2 depict the proton NMR spectrum and LC-CAD chromatogram of Lipid 4. FIG. 4C-1 depicts proton NMR spectrum of Lipid 4; FIG. 4C-2 depicts the LC-CAD chromatogram L of Lipid 4.

FIGS. 4D-1 and 4D-2 depict the proton NMR spectrum and LC-CAD chromatogram of Lipid 5A. FIG. 4D-1 depicts proton NMR spectrum of Lipid 5A; FIG. 4D-2 depicts the LC-CAD chromatogram of Lipid 5A.

FIGS. 4E-1 and 4E-2 depict the proton NMR spectrum and LC-CAD chromatogram of Lipid 6. FIG. 4E-1 depicts proton NMR spectrum of Lipid 6; FIG. 4E-2 depicts the LC-CAD chromatogram of Lipid 6.

FIGS. 4F-1 and 4F-2 depict the proton NMR spectrum and LC-CAD chromatogram of Lipid 7. FIG. 4F-1 depicts proton NMR spectrum of Lipid 7; FIG. 4F-2 depicts the LC-CAD chromatogram of Lipid 7.

FIGS. 4G-1 and 4G-2 depict the proton NMR spectrum and LC-CAD chromatogram of Lipid 2. FIG. 4G-1 depicts proton NMR spectrum of Lipid 2; FIG. 4G-2 depicts the LC-CAD chromatogram of Lipid 2.

FIGS. 4H-1 and 4H-2 depict the proton NMR spectrum and LC-CAD chromatogram of Lipid 8. FIG. 4H-1 depicts proton NMR spectrum of Lipid 8; FIG. 4H-2 depicts the LC-CAD chromatogram of Lipid 8.

FIGS. 4I-1 and 4I-2 depict the proton NMR spectrum and LC-CAD chromatogram of Lipid 9. FIG. 4I-1 depicts proton NMR spectrum of Lipid 9; FIG. 4I-2 depicts the LC-CAD chromatogram of Lipid 9.

FIGS. 4J-1 and 4J-2 depict the proton NMR spectrum and LC-CAD chromatogram of Lipid 10A. FIG. 4J-1 depicts proton NMR spectrum of Lipid 10A; FIG. 4J-2 depicts the LC-CAD chromatogram of Lipid 10A.

FIGS. 4K-1 and 4K-2 depict the proton NMR spectrum and LC-CAD chromatogram of Lipid 11A. FIG. 4K-1 depicts proton NMR spectrum of Lipid 11A; FIG. 4K-2 depicts the LC-CAD chromatogram of Lipid 11A.

FIGS. 4L-1 and 4L-2 depict the proton NMR spectrum and LC-CAD chromatogram of Lipid 12. FIG. 4L-1 depicts proton NMR spectrum of Lipid 12; FIG. 4L-2 depicts the LC-CAD chromatogram of Lipid 12.

FIGS. 4M-1 and 4M-2 depict the proton NMR spectrum and LC-CAD chromatogram of Lipid 13. FIG. 4M-1 depicts proton NMR spectrum of Lipid 13; FIG. 4M-2 depicts the LC-CAD chromatogram of Lipid 13.

FIGS. 4N-1 and 4N-2 depict the proton NMR spectrum and LC-CAD chromatogram of Lipid 15. FIG. 4N-1 depicts proton NMR spectrum of Lipid 15; FIG. 4N-2 depicts the LC-CAD chromatogram of Lipid 15.

FIGS. 40-1 and 40-2 depict the proton NMR spectrum and LC-CAD chromatogram of Lipid 16. FIG. 40-1 depicts proton NMR spectrum of Lipid 16; FIG. 40-2 depicts the LC-CAD of Lipid 16.

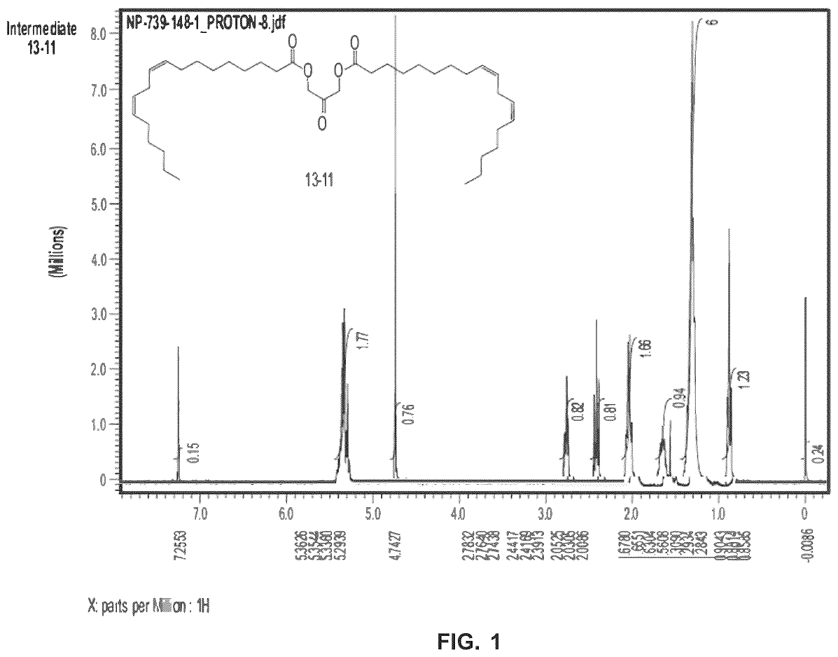

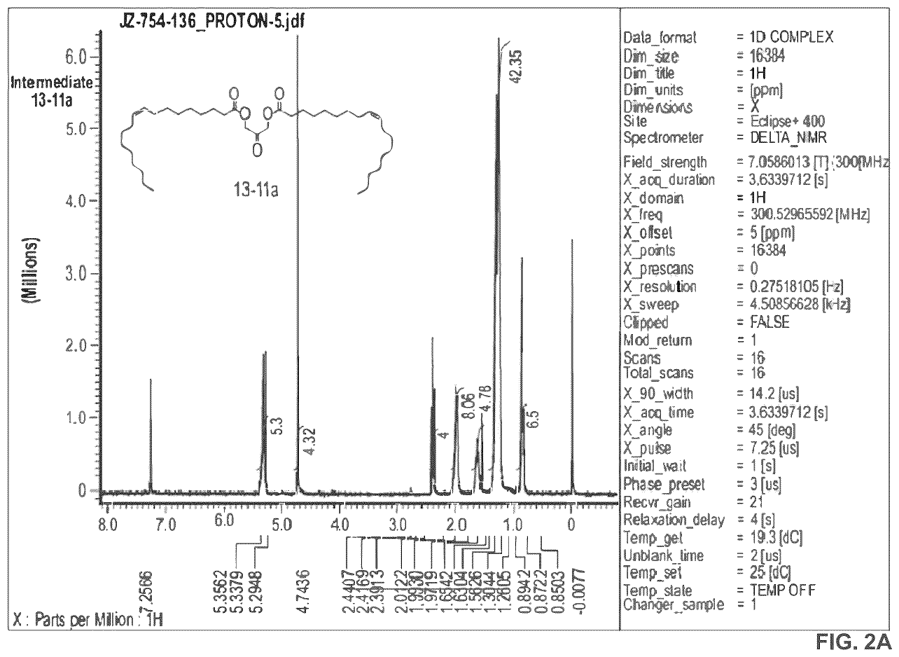

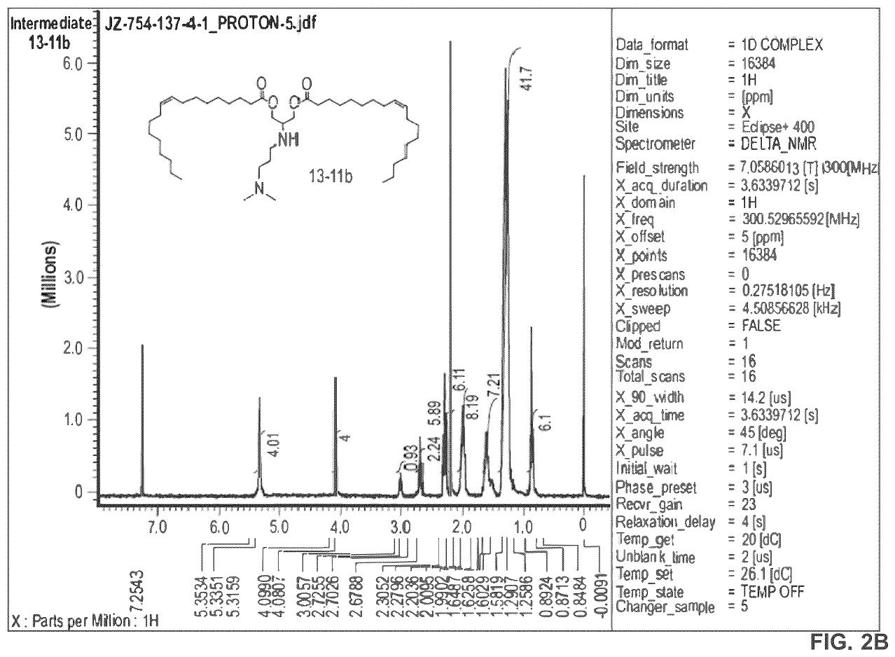

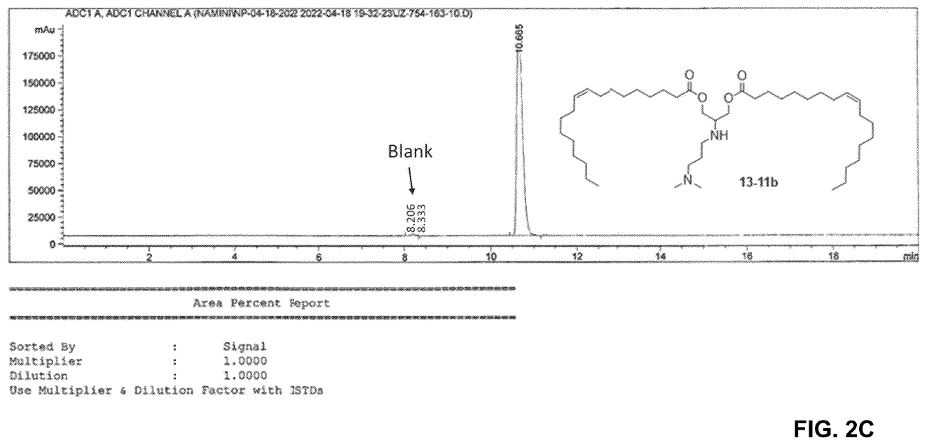

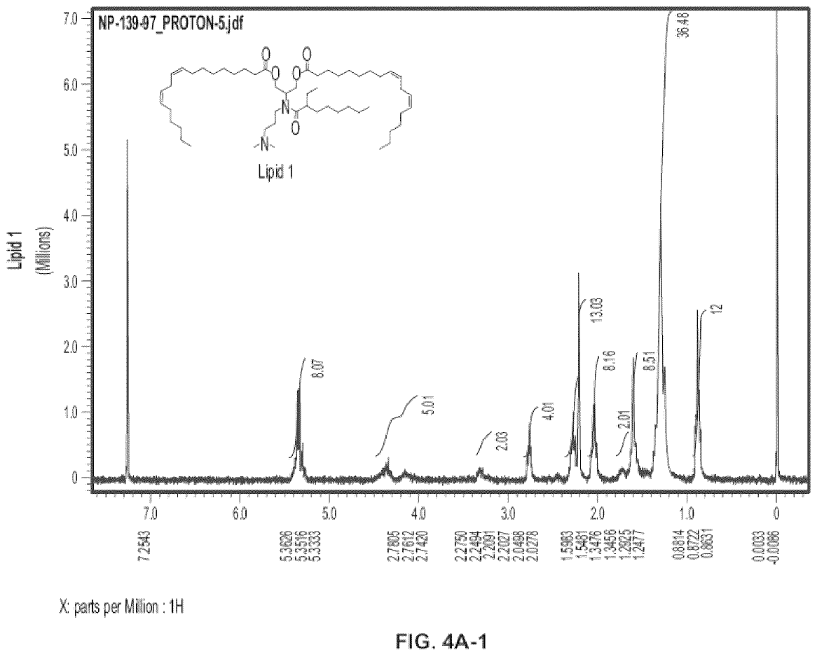

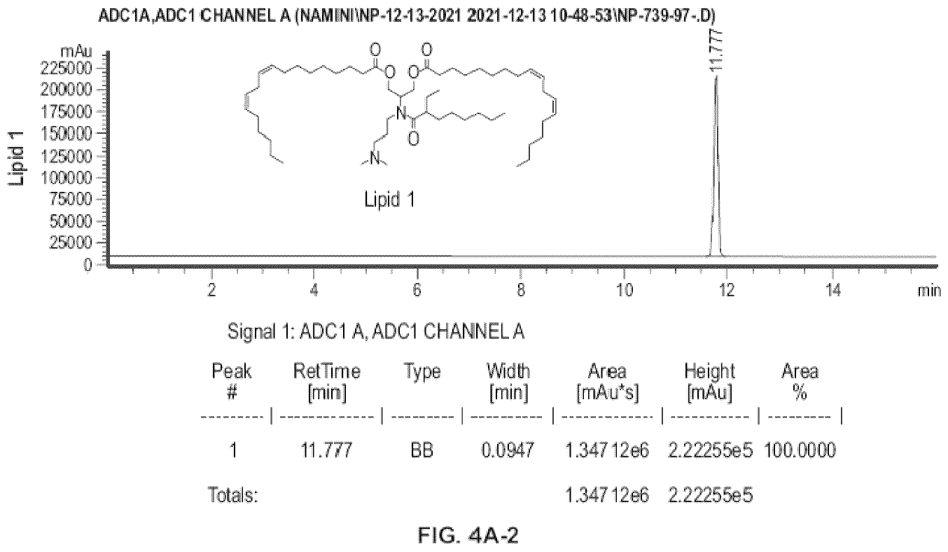

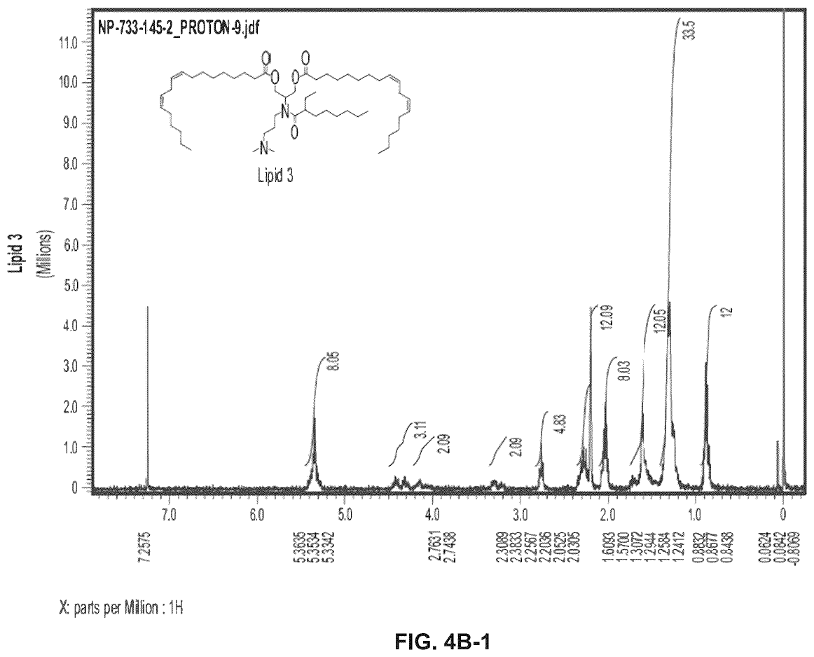

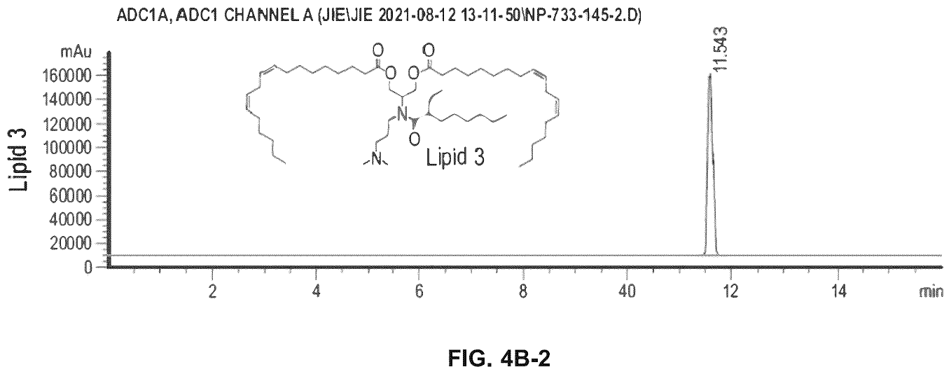

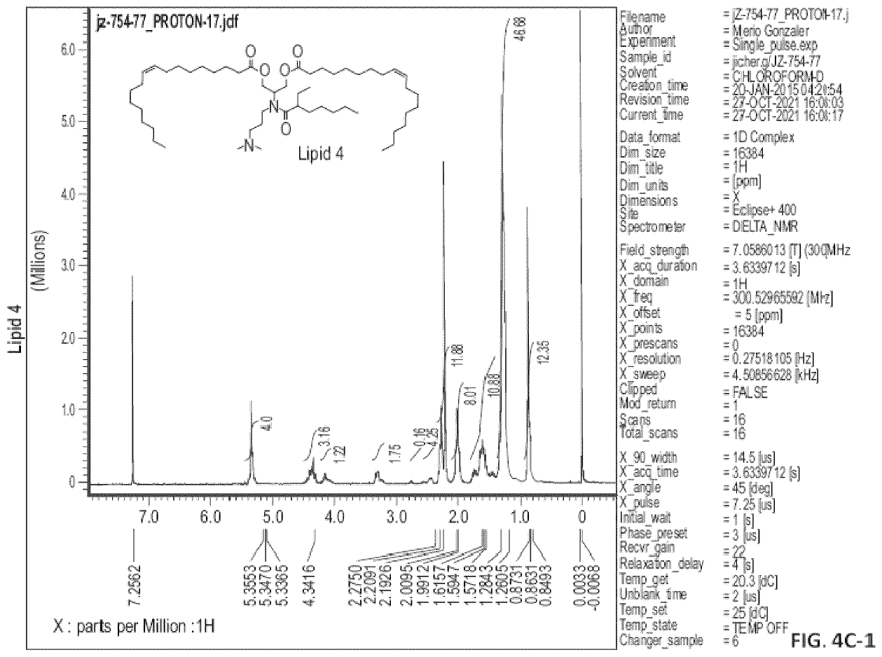

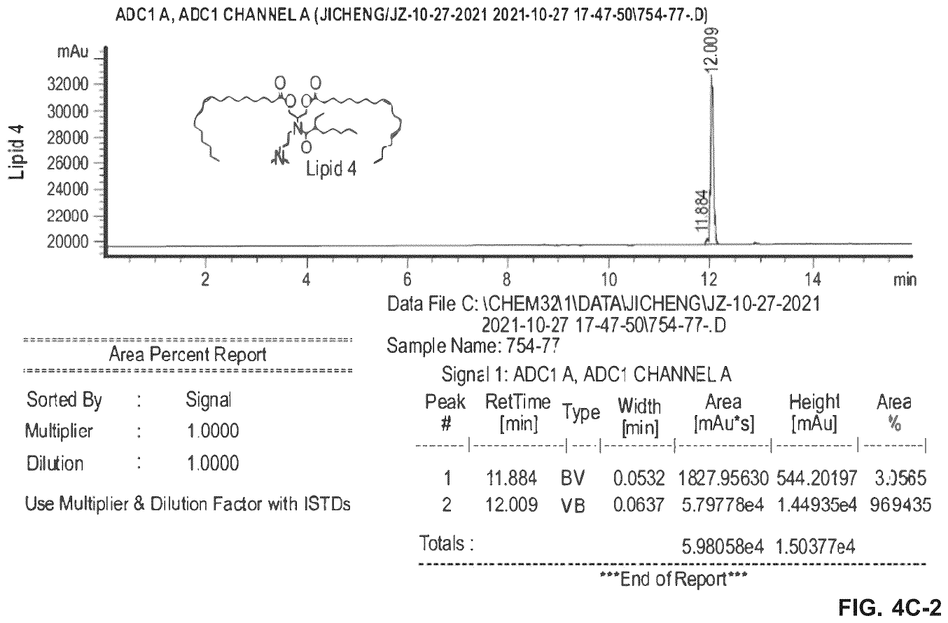

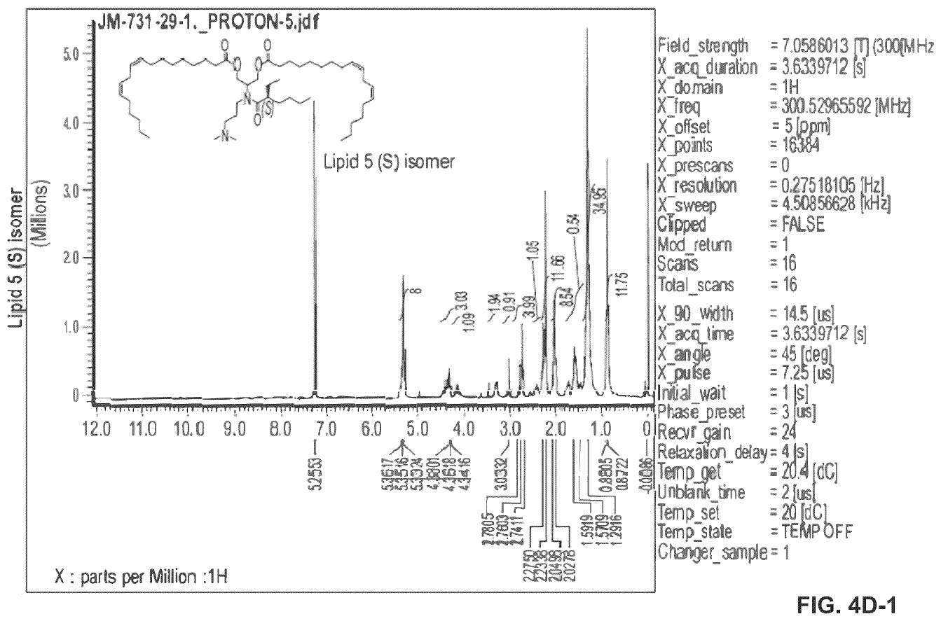

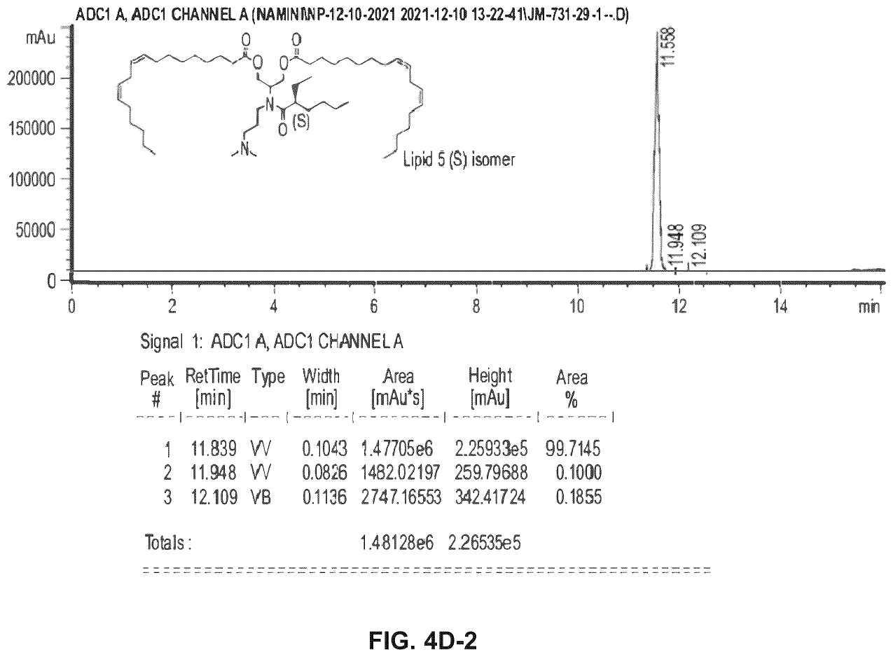

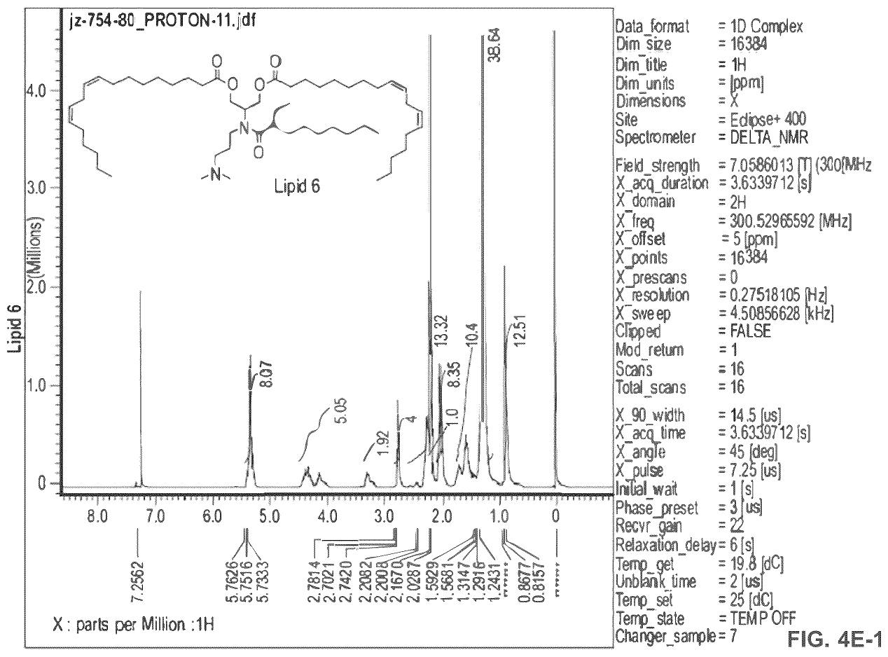

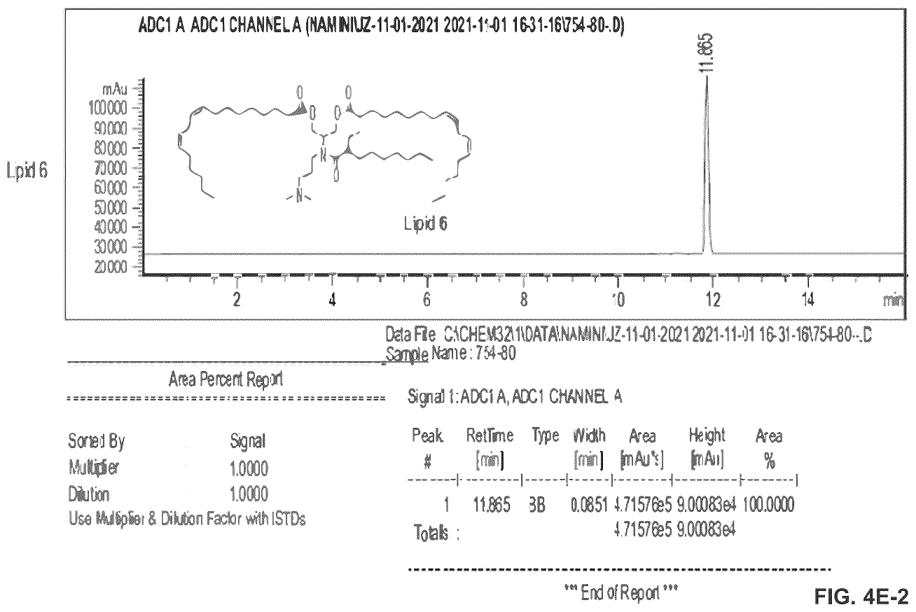

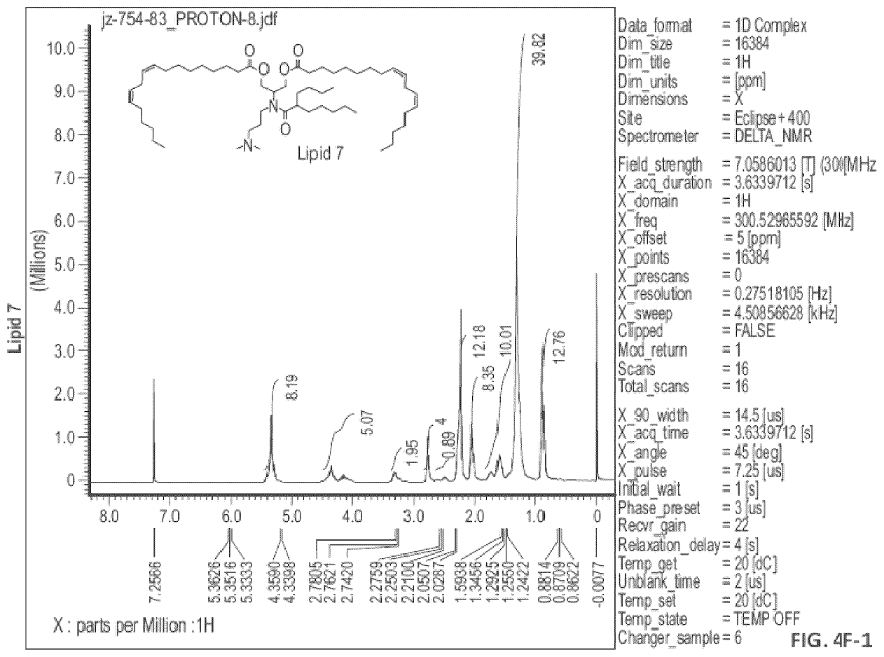

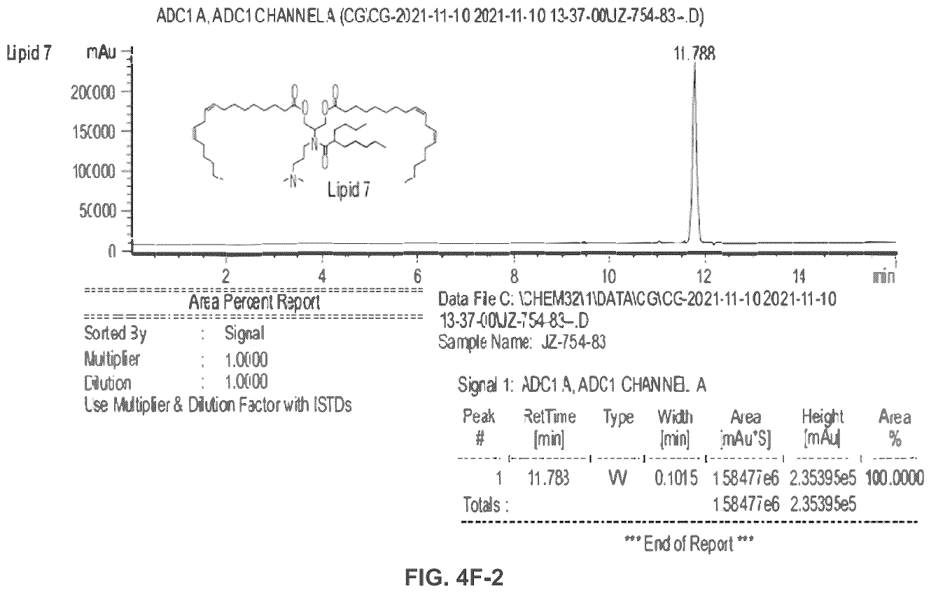

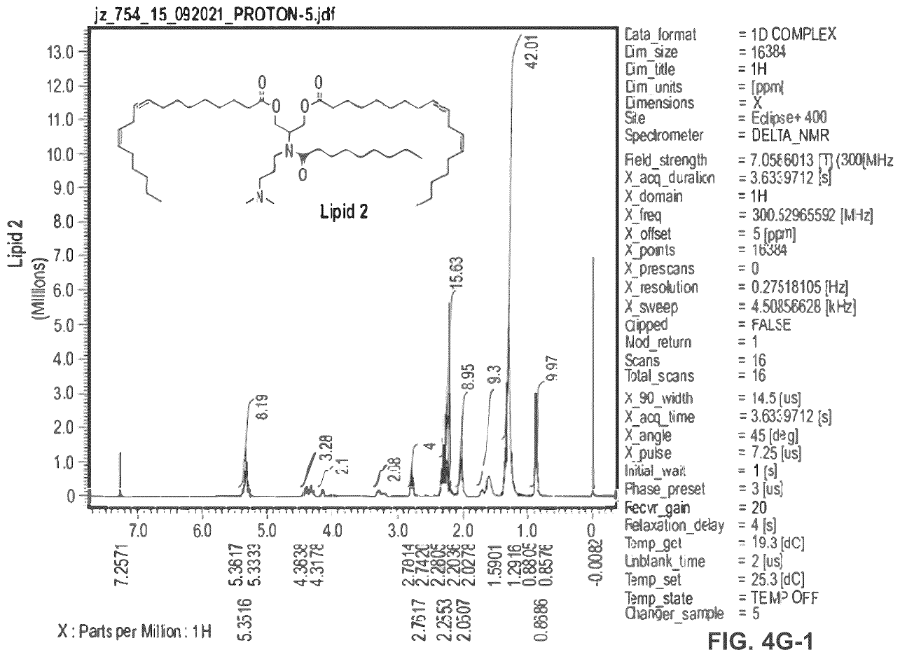

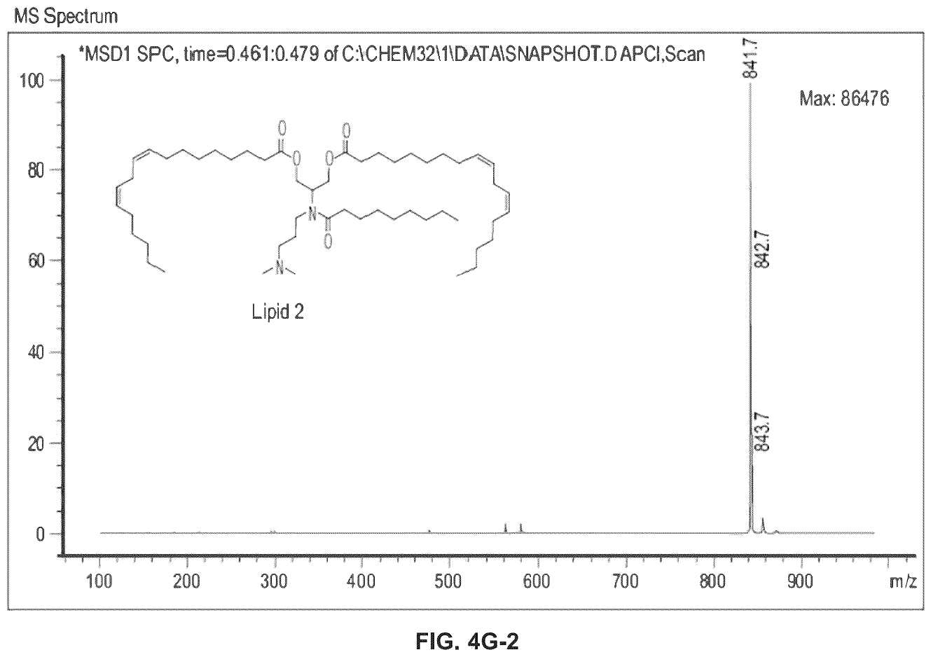

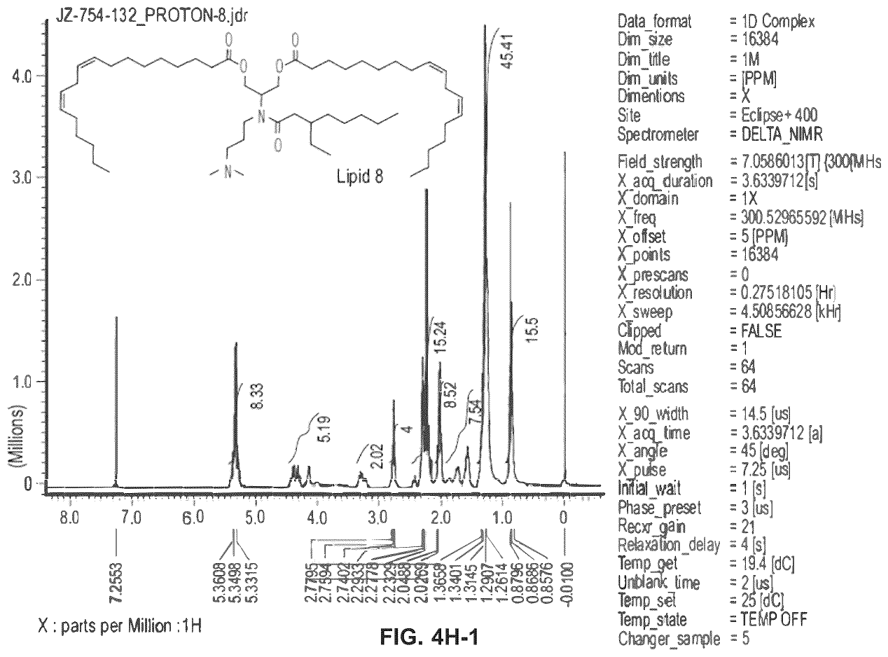

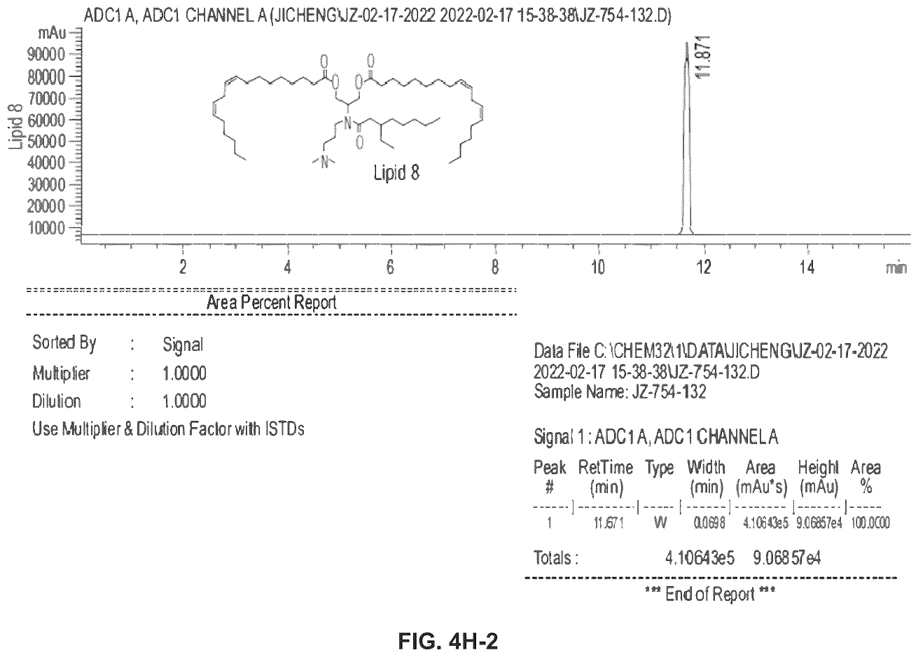

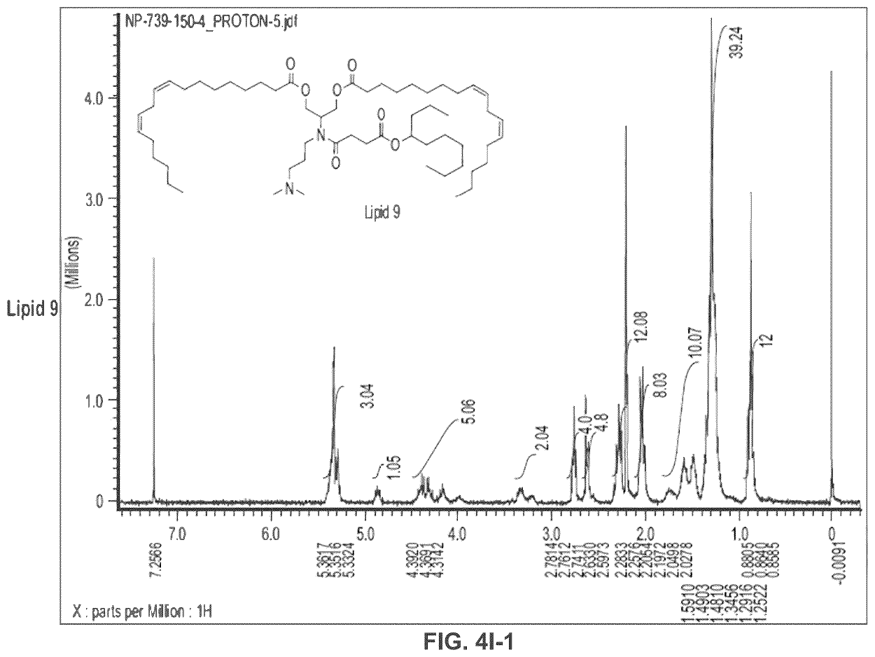

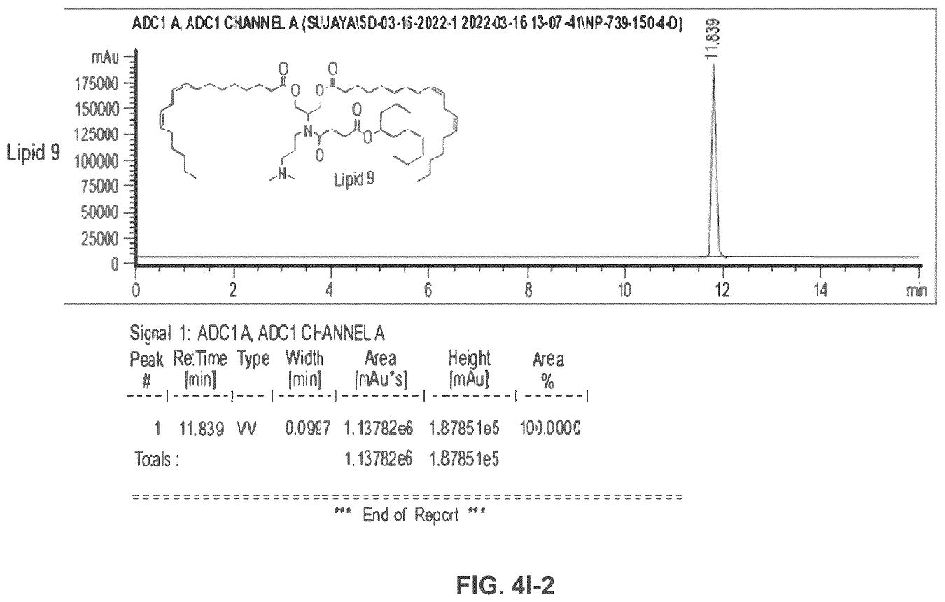

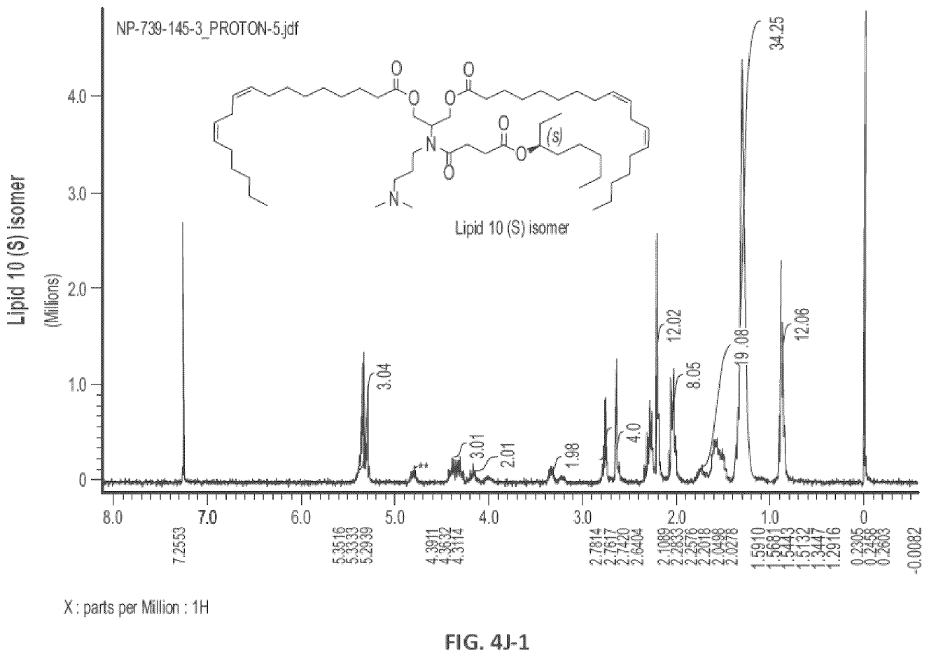

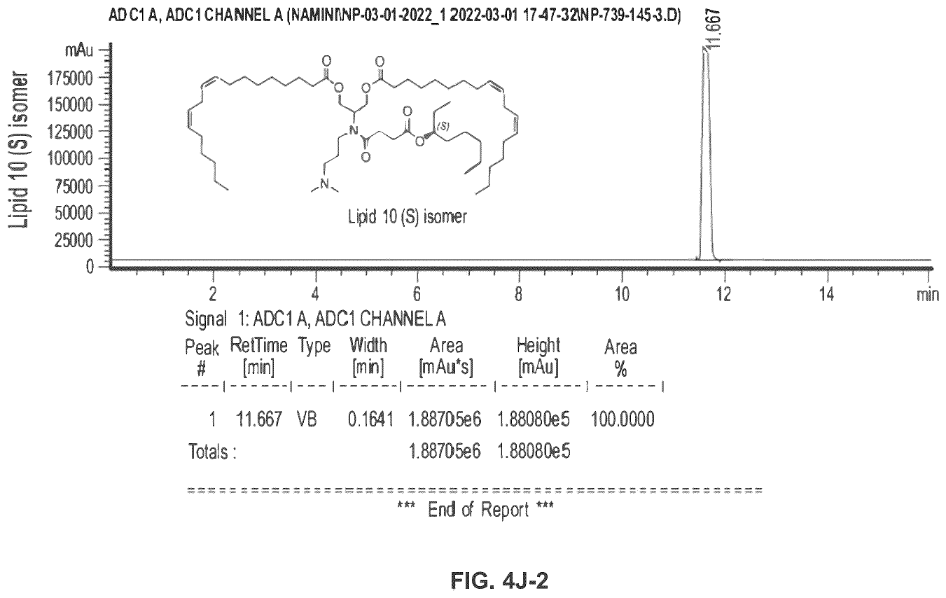

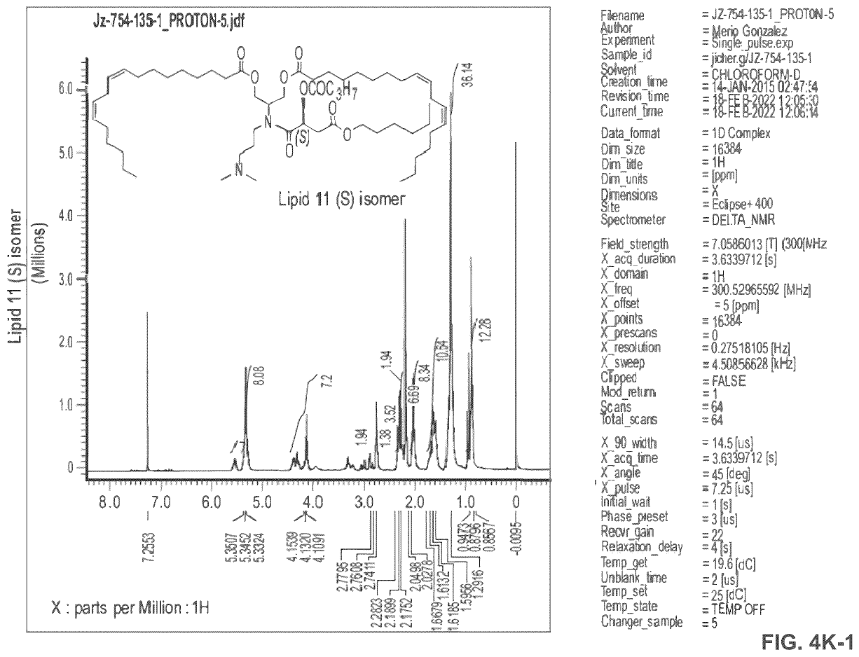

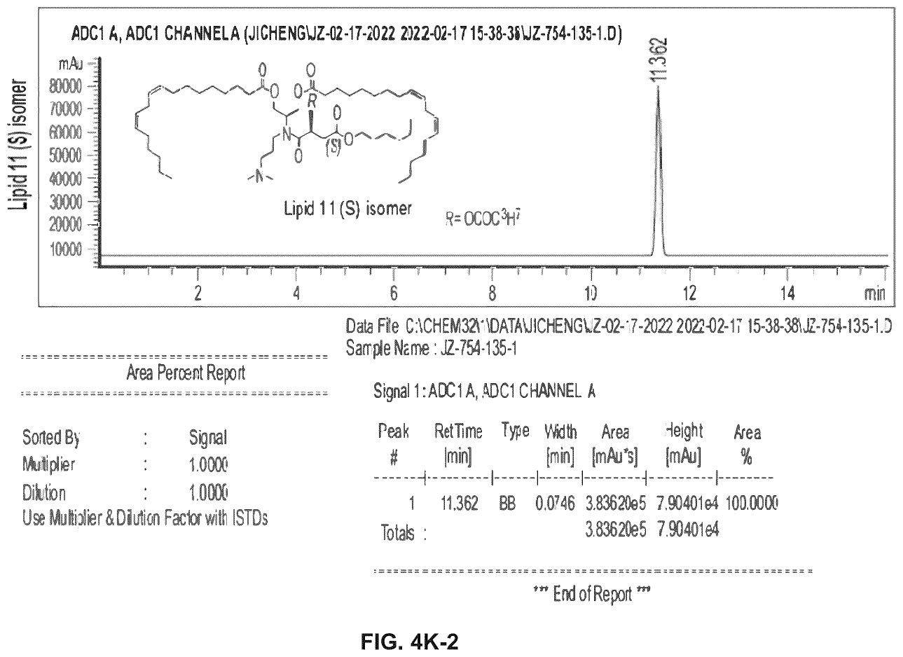

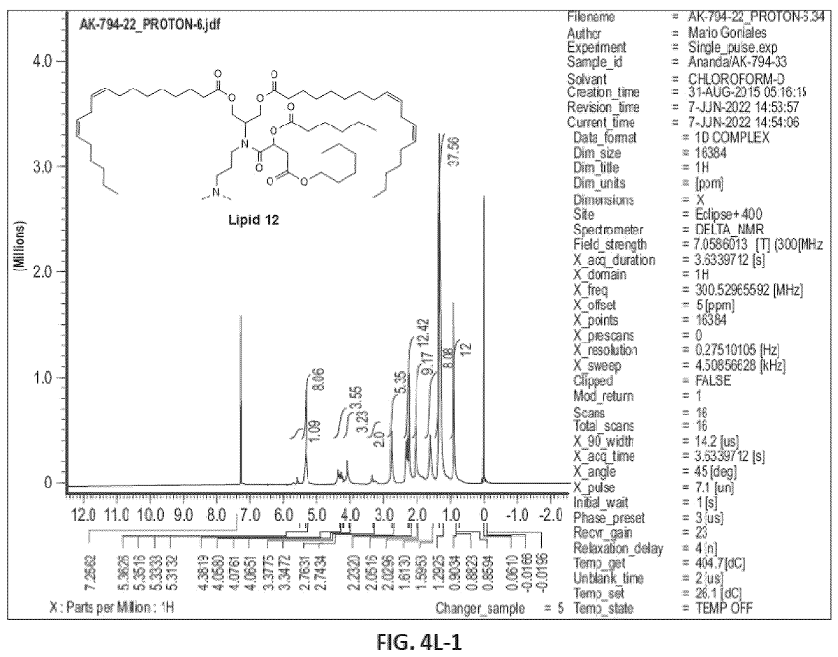

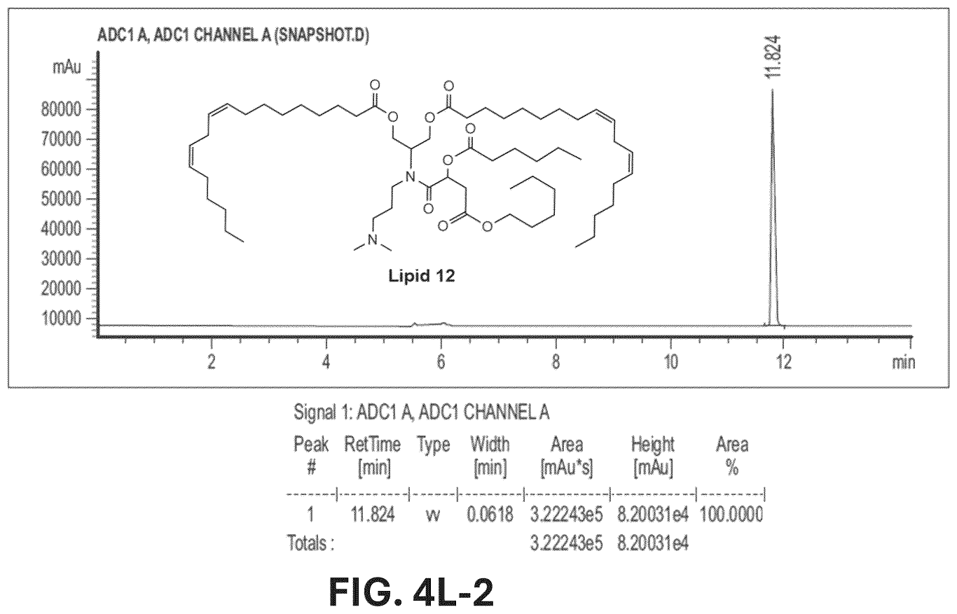

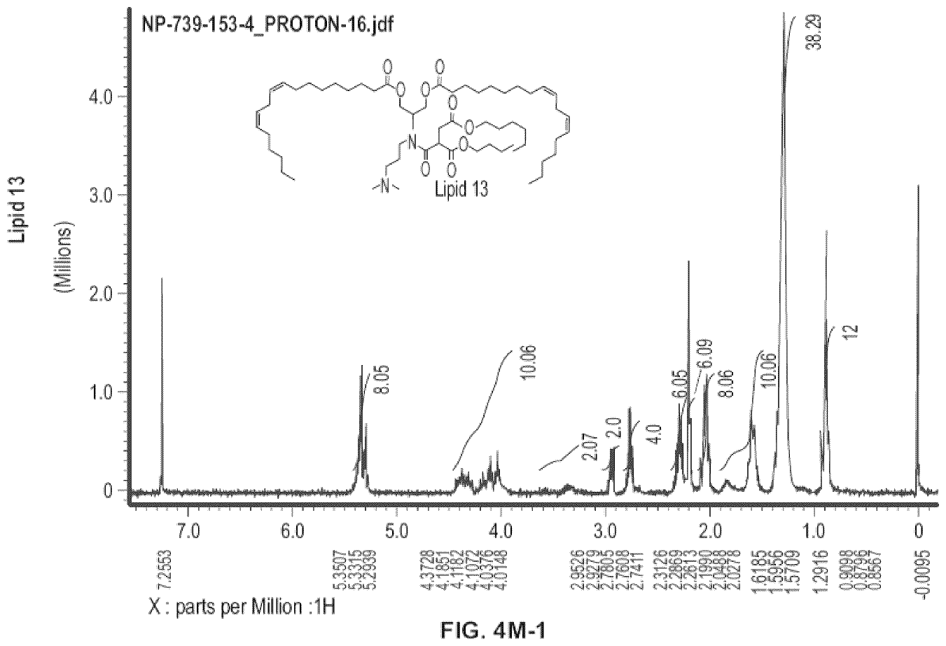

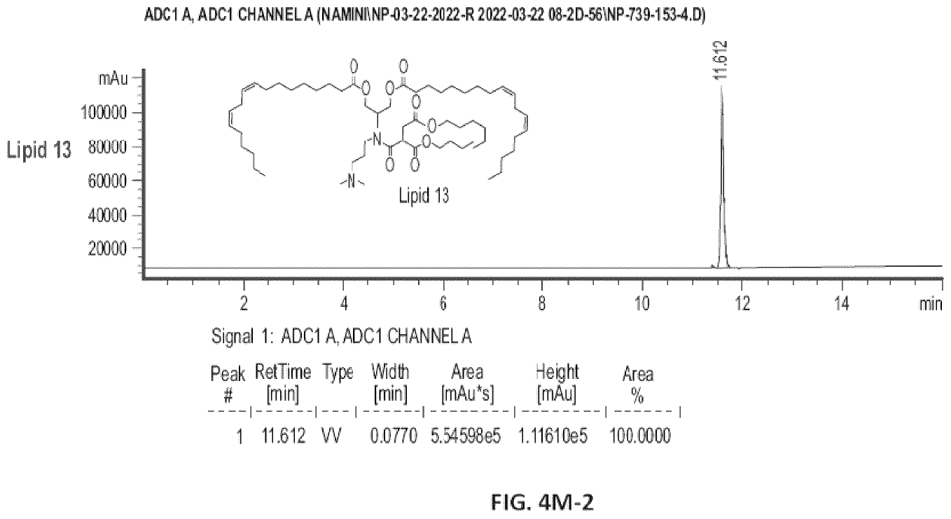

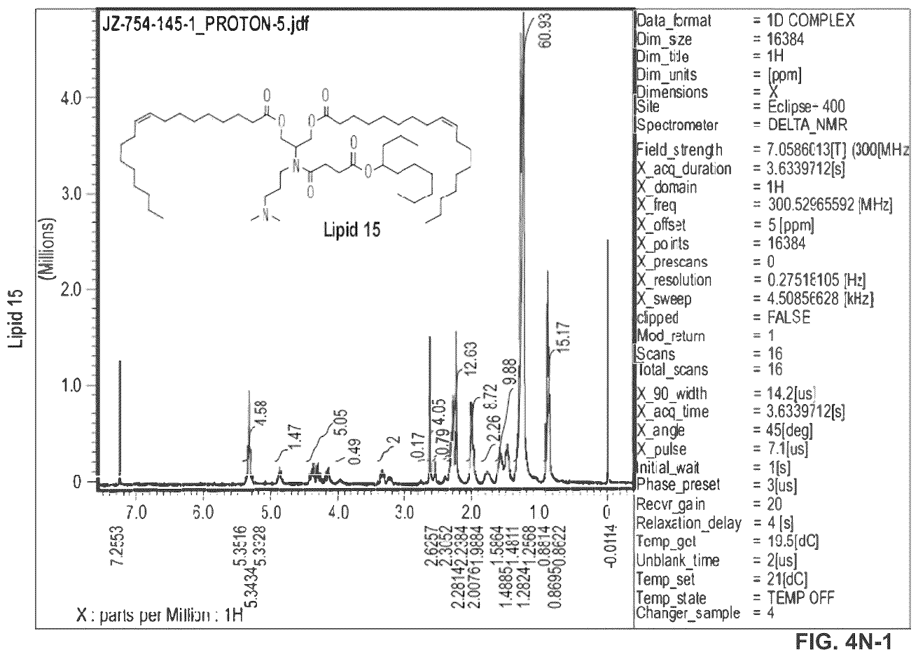

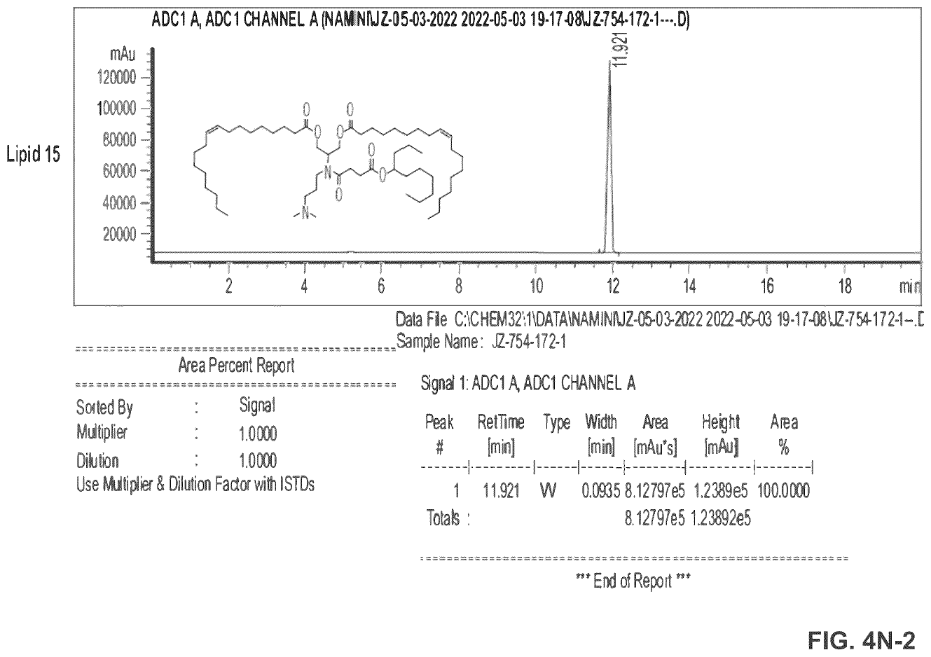

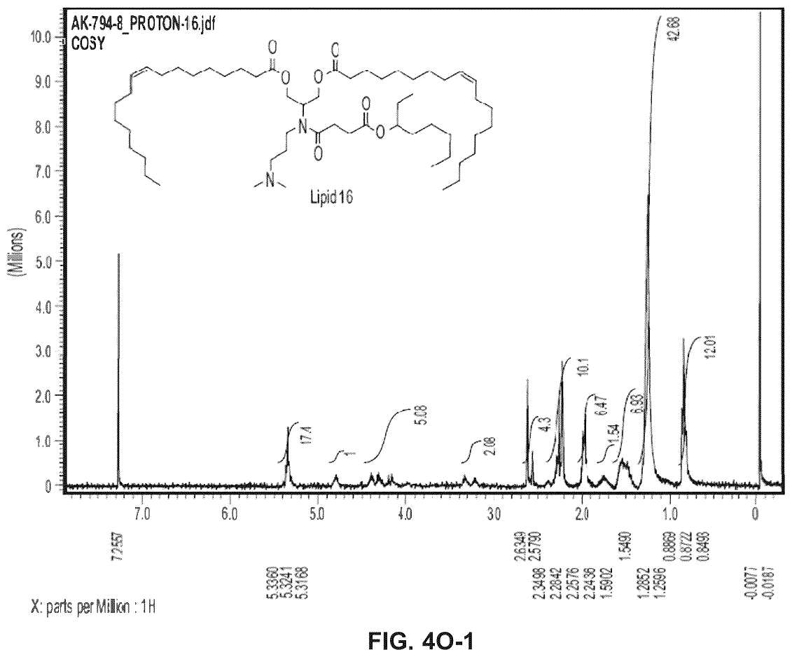

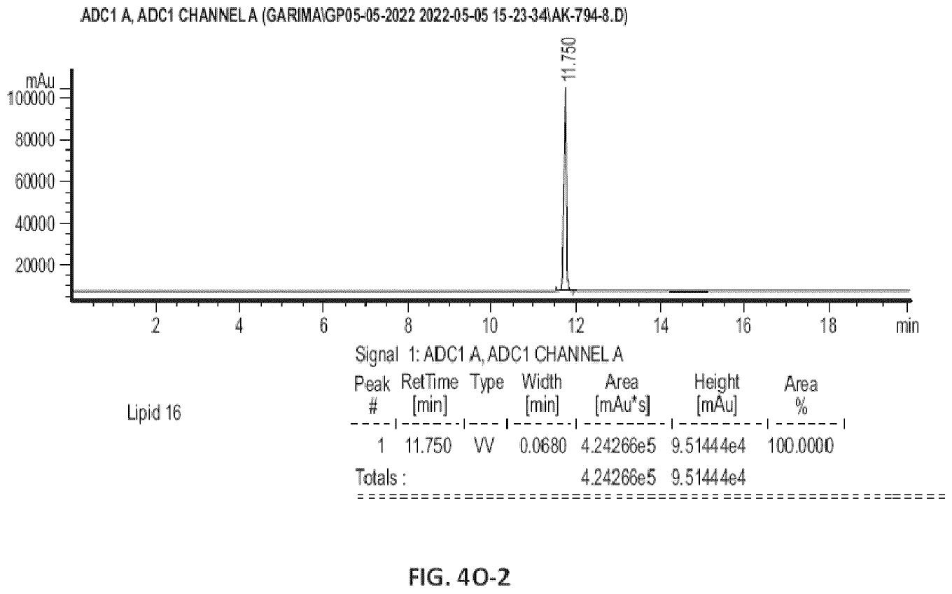

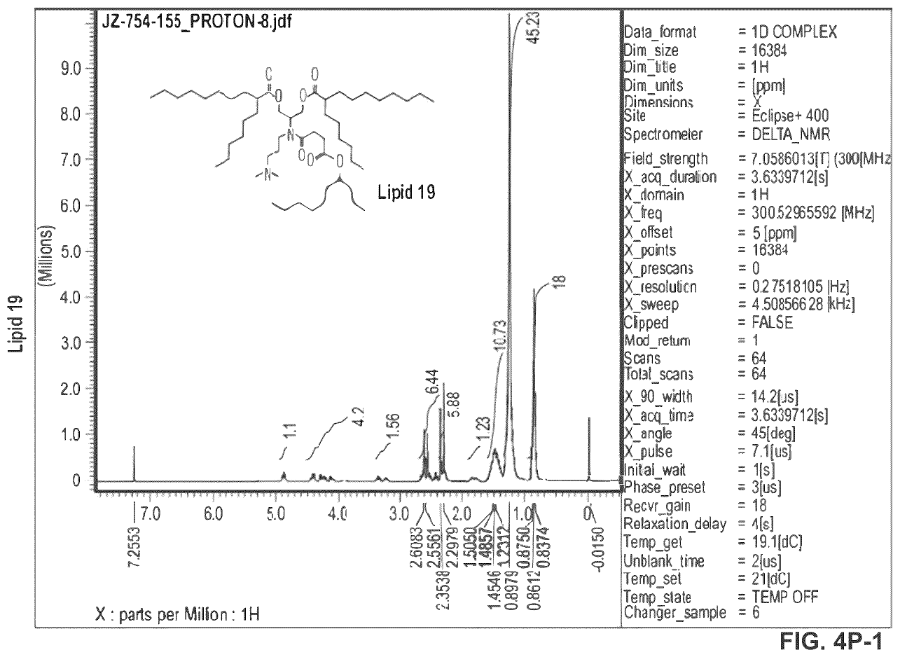

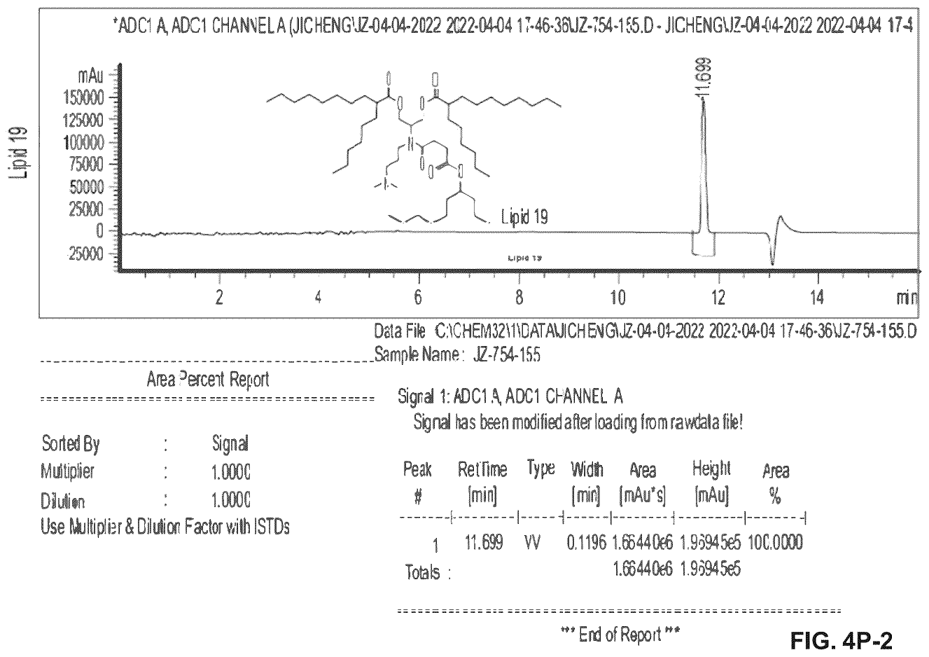

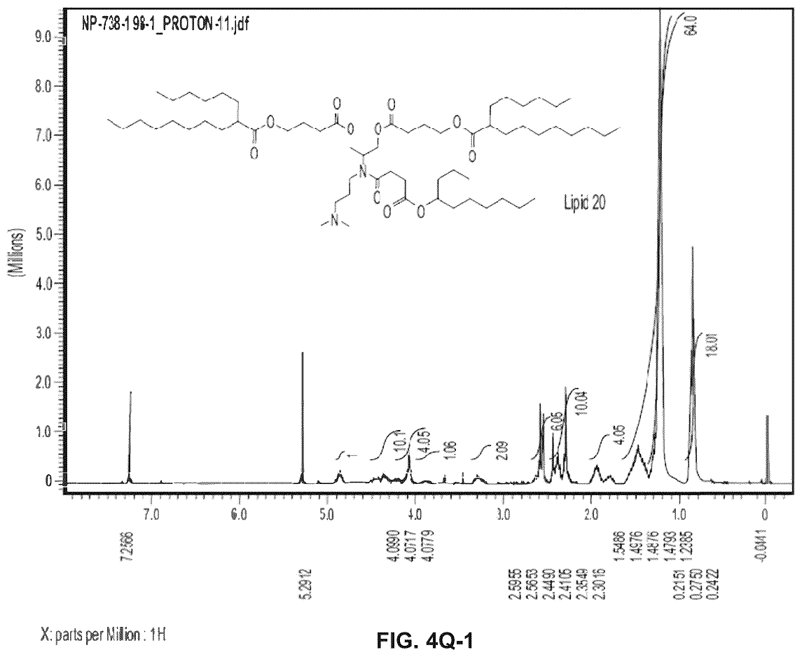

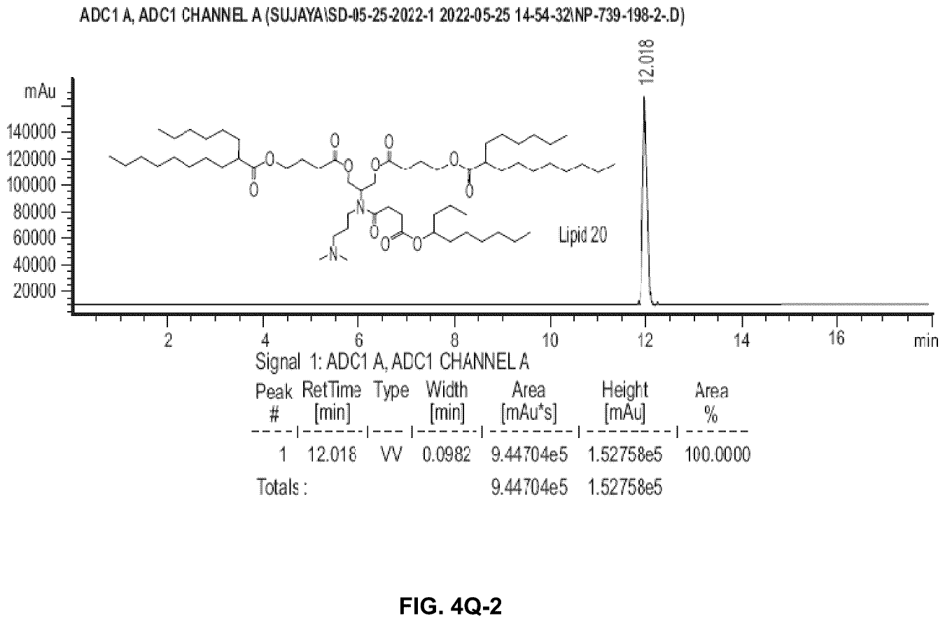

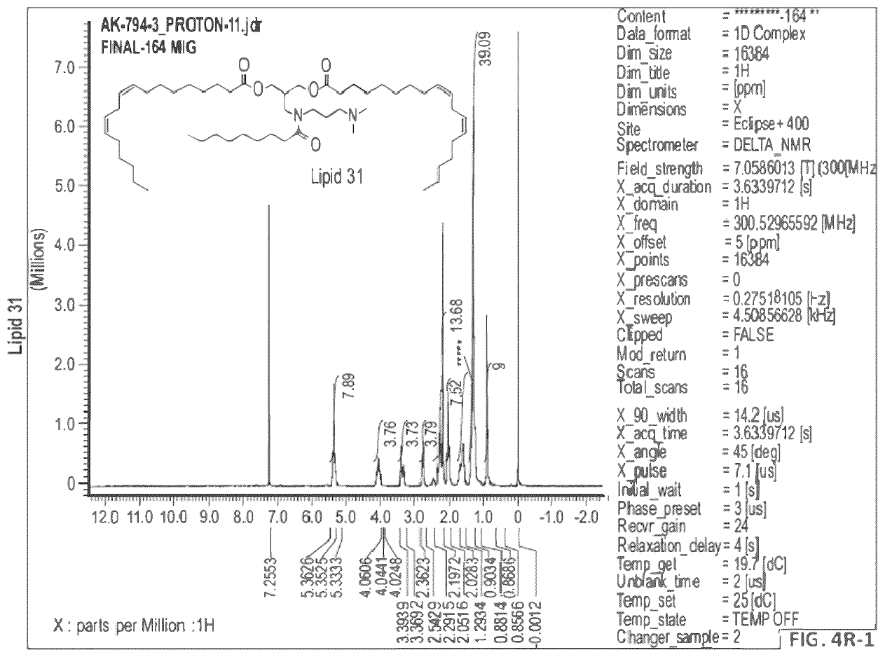

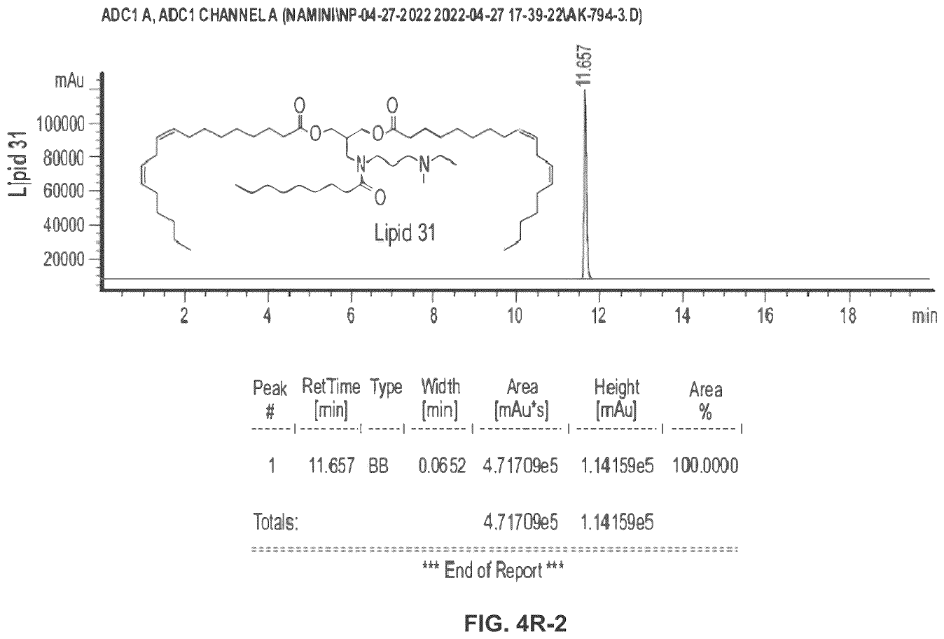

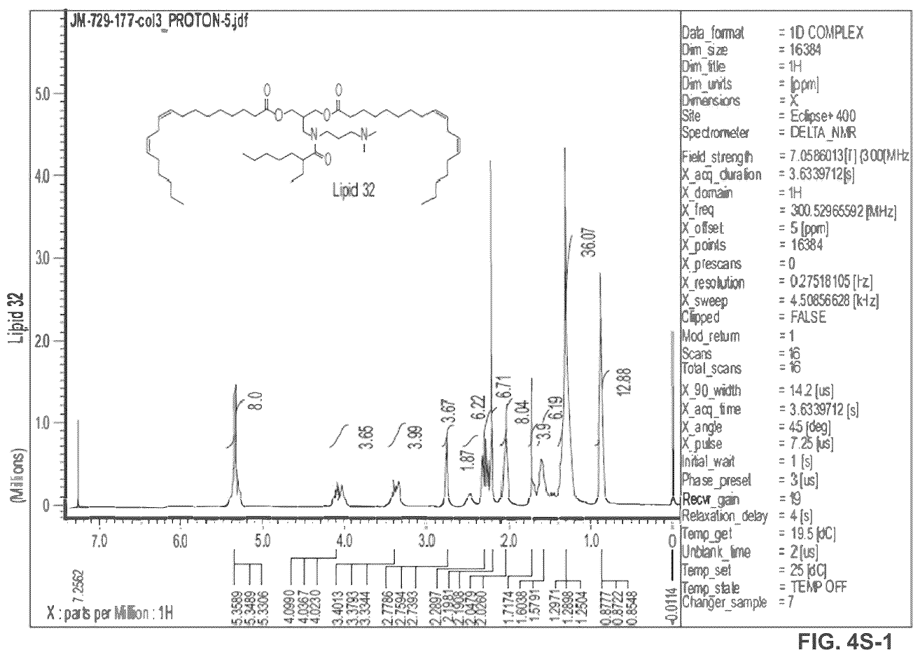

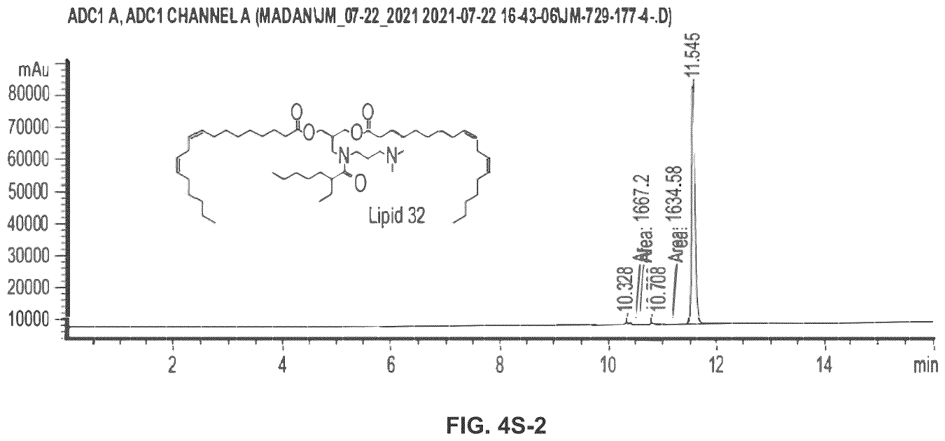

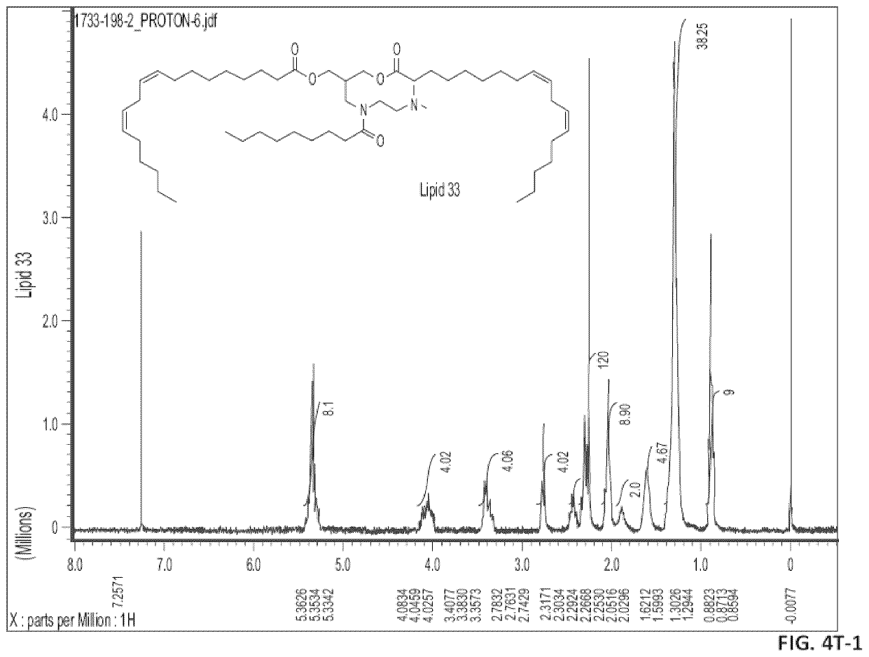

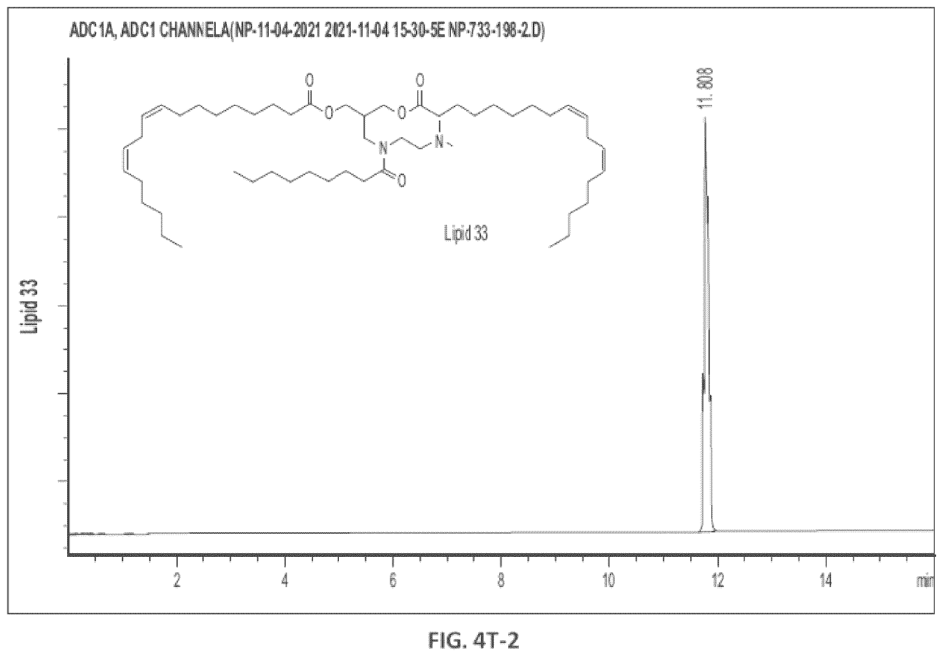

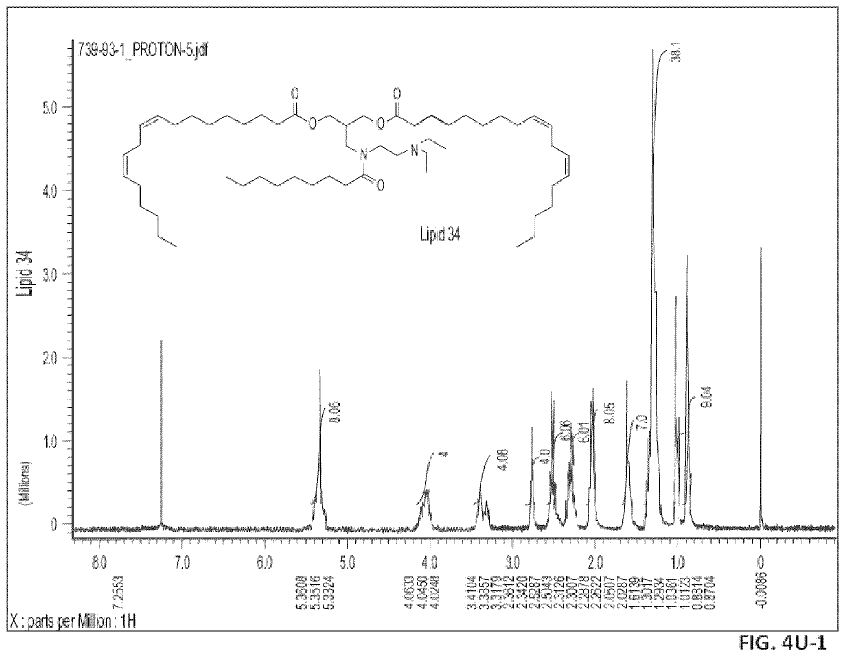

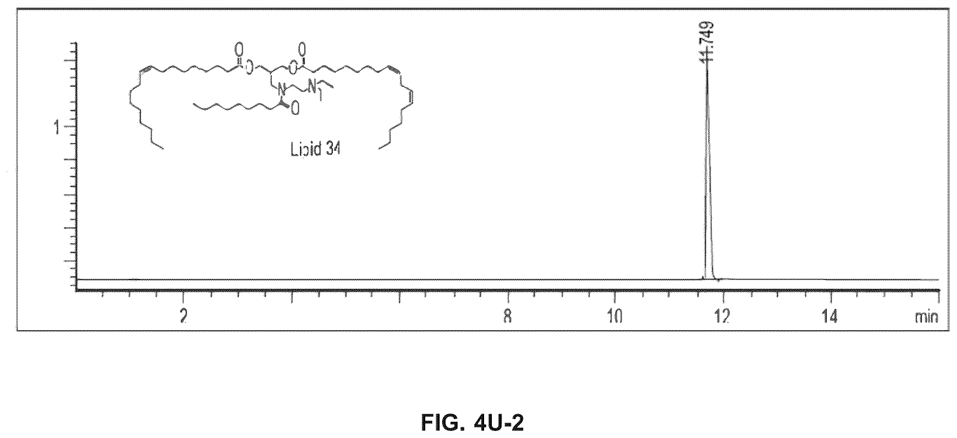

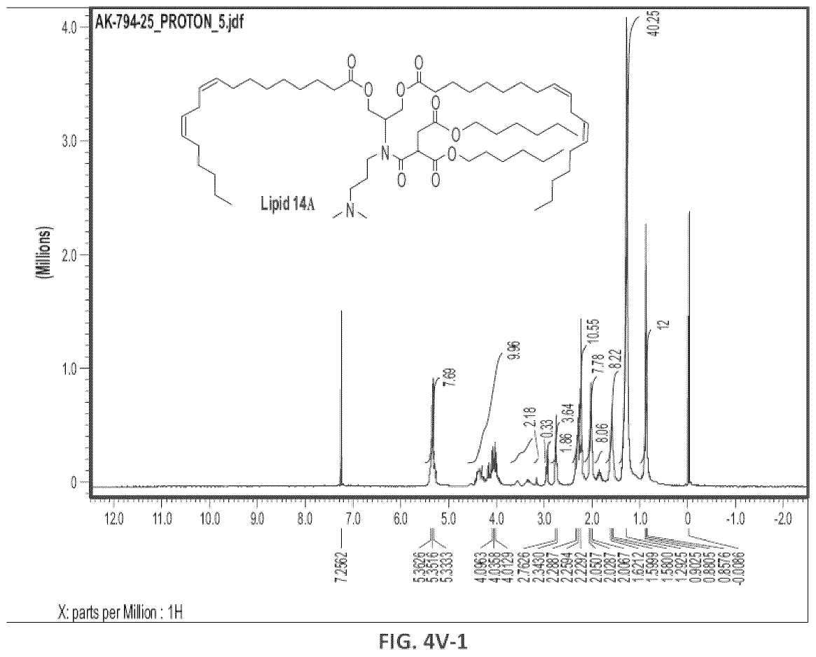

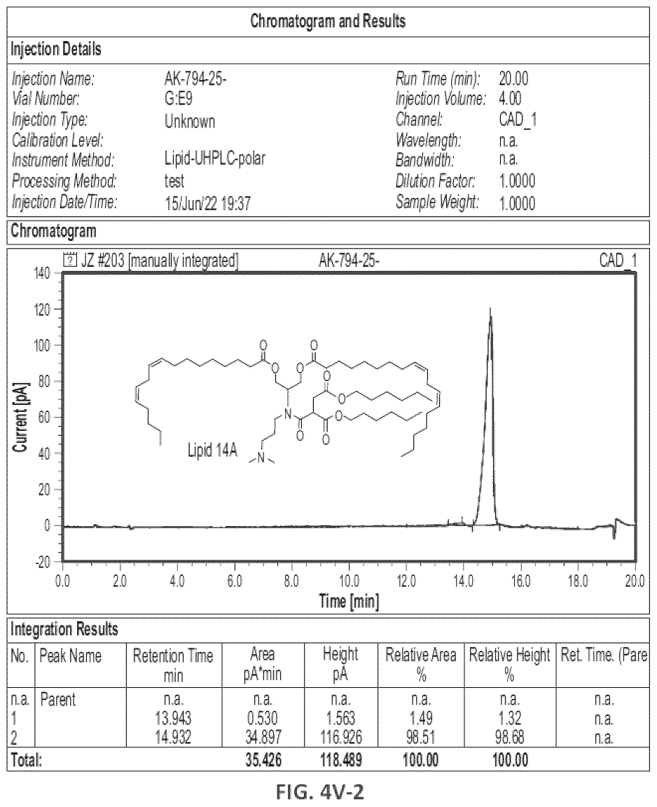

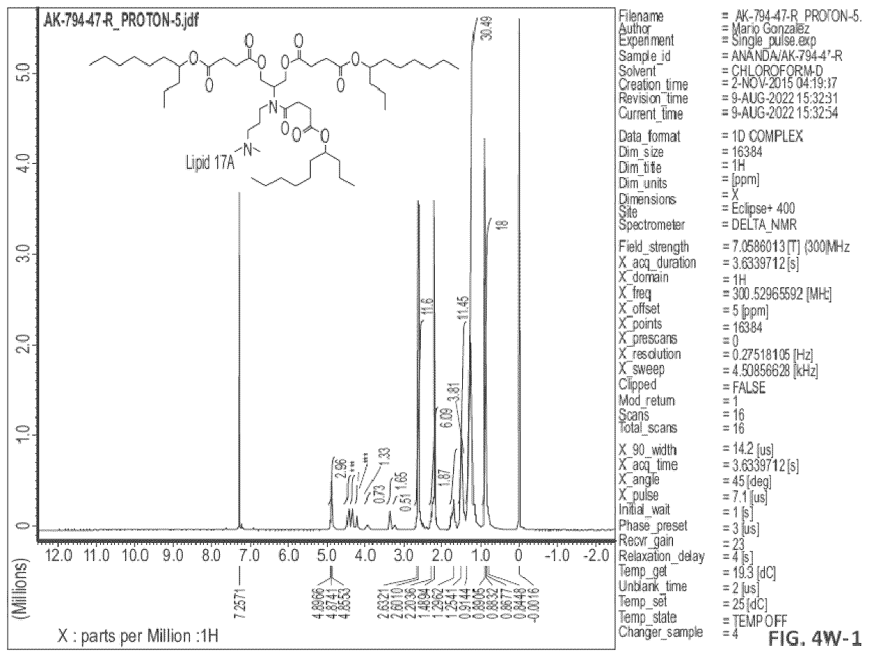

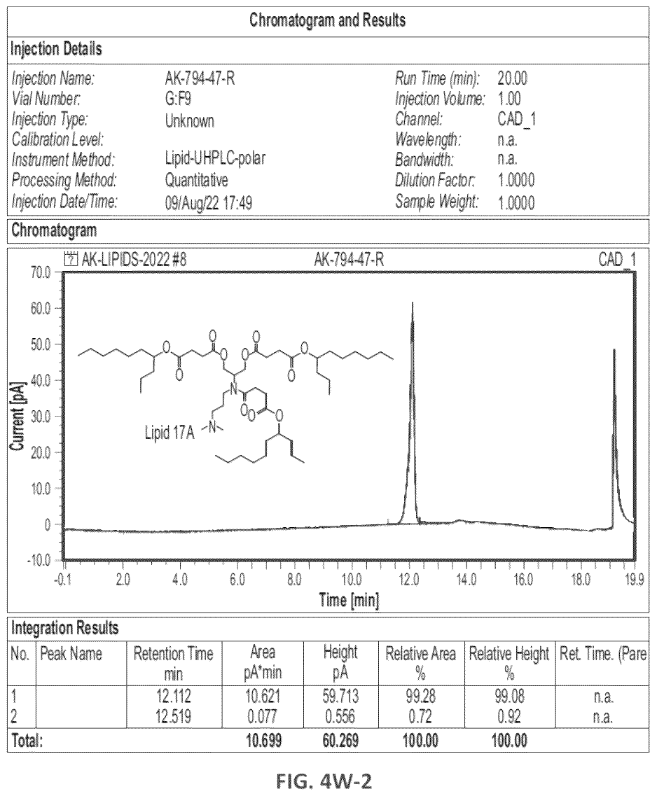

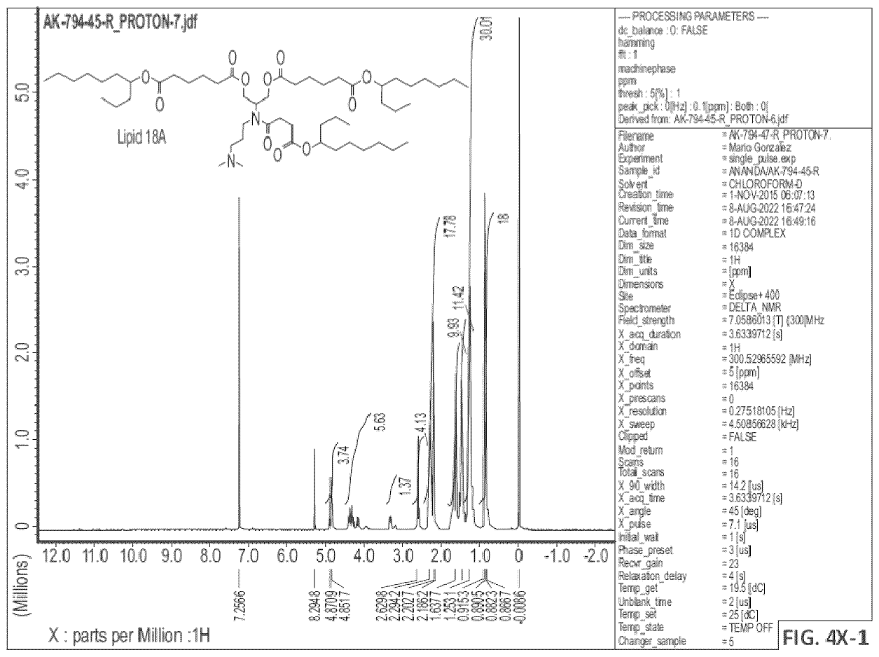

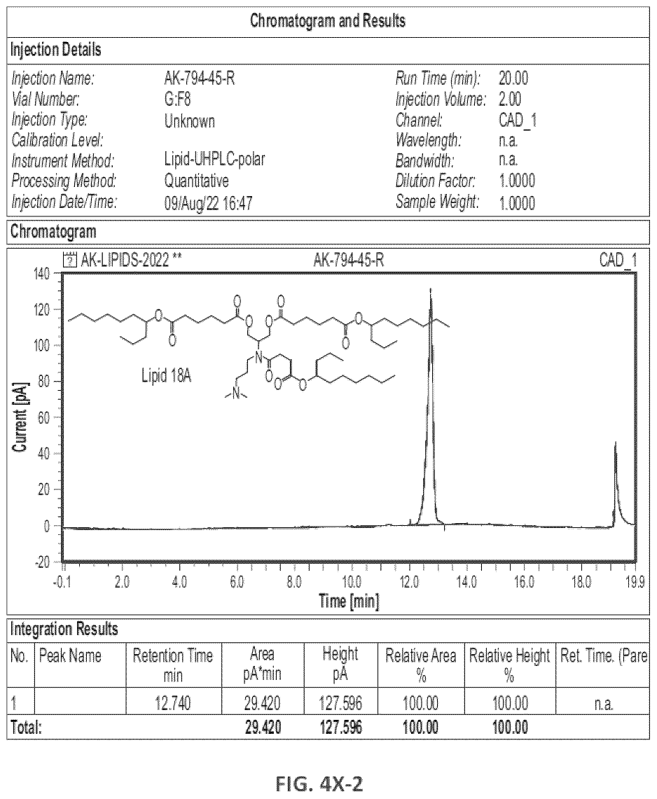

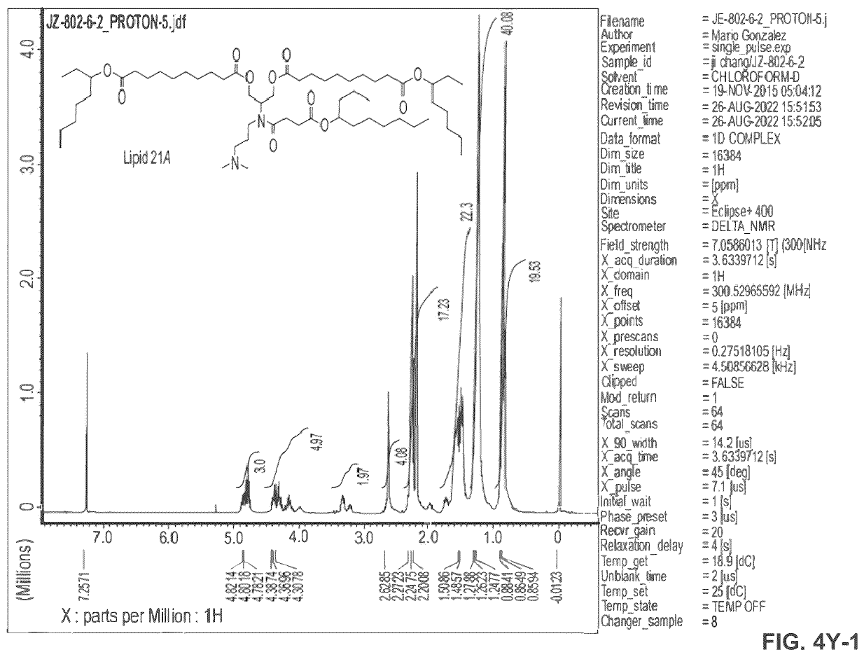

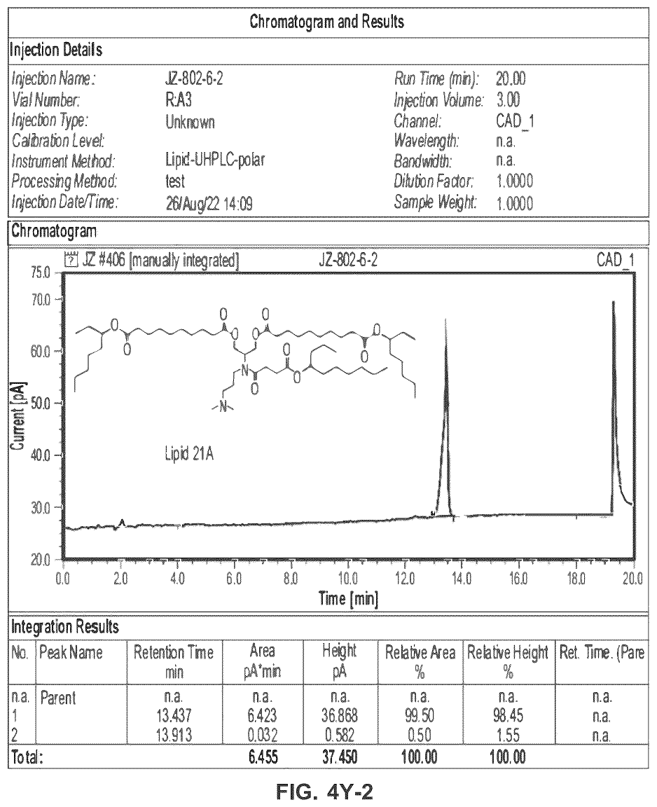

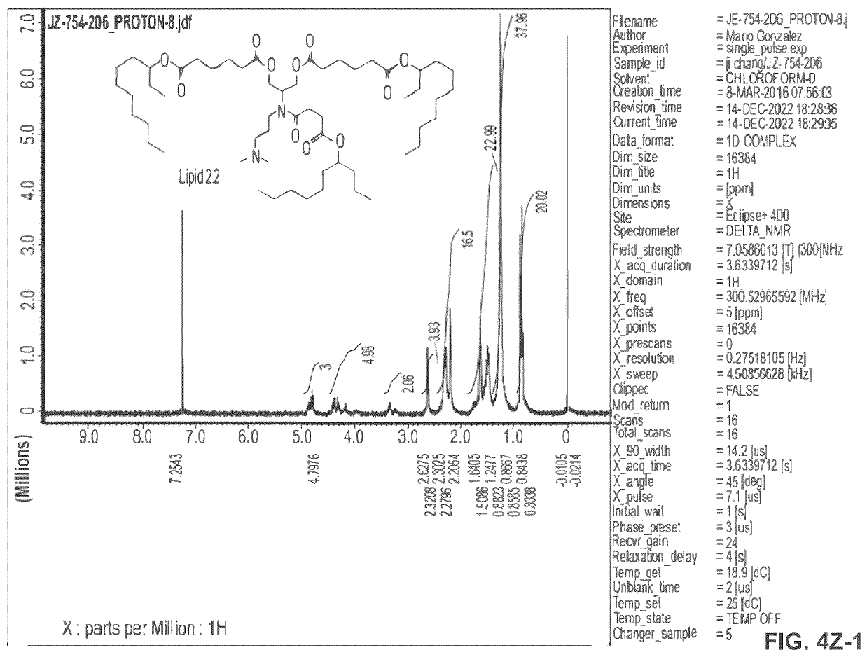

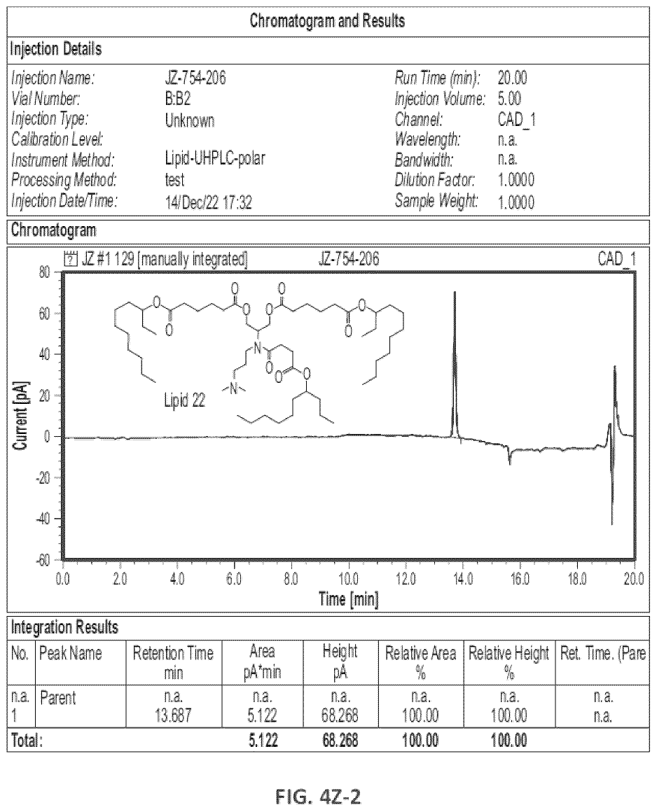

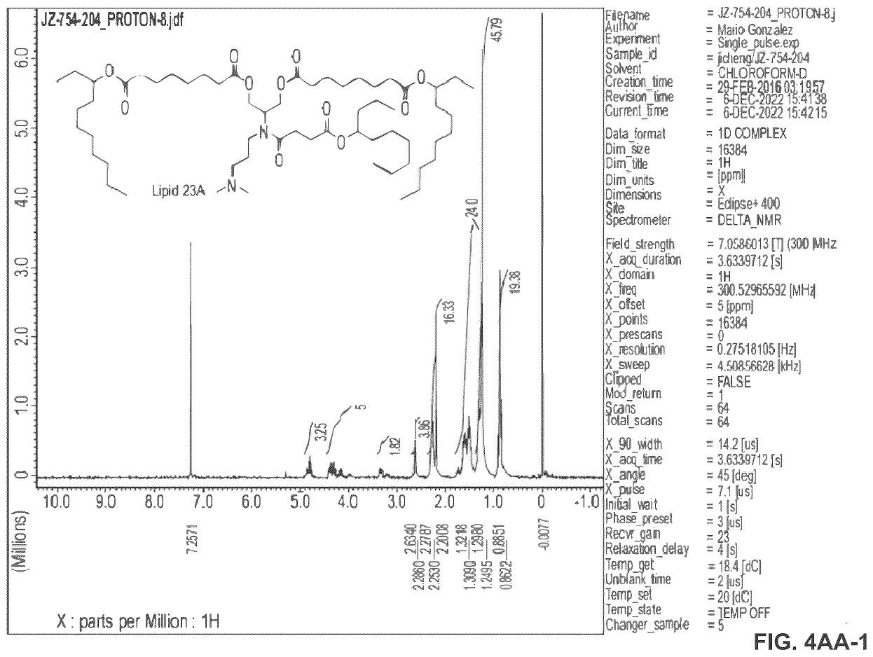

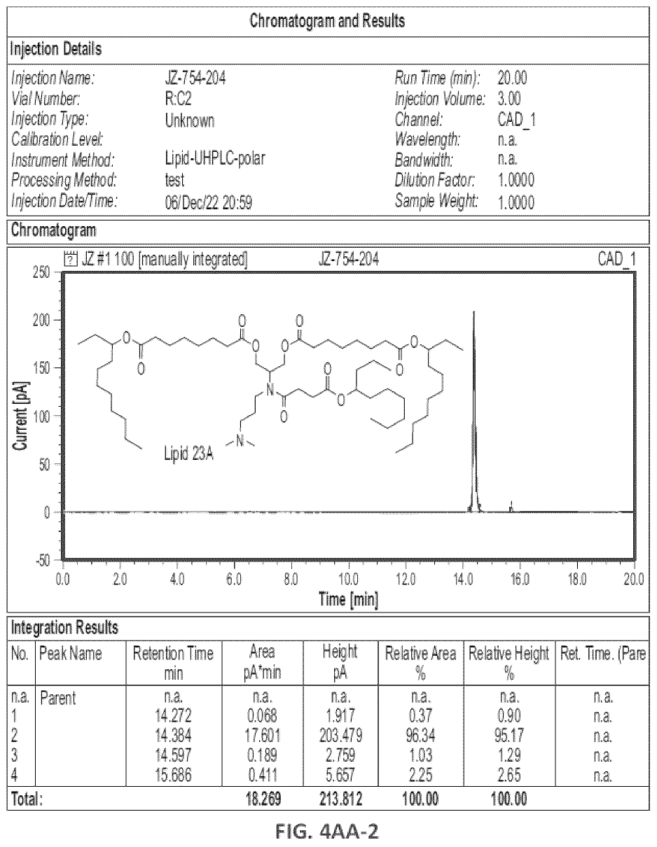

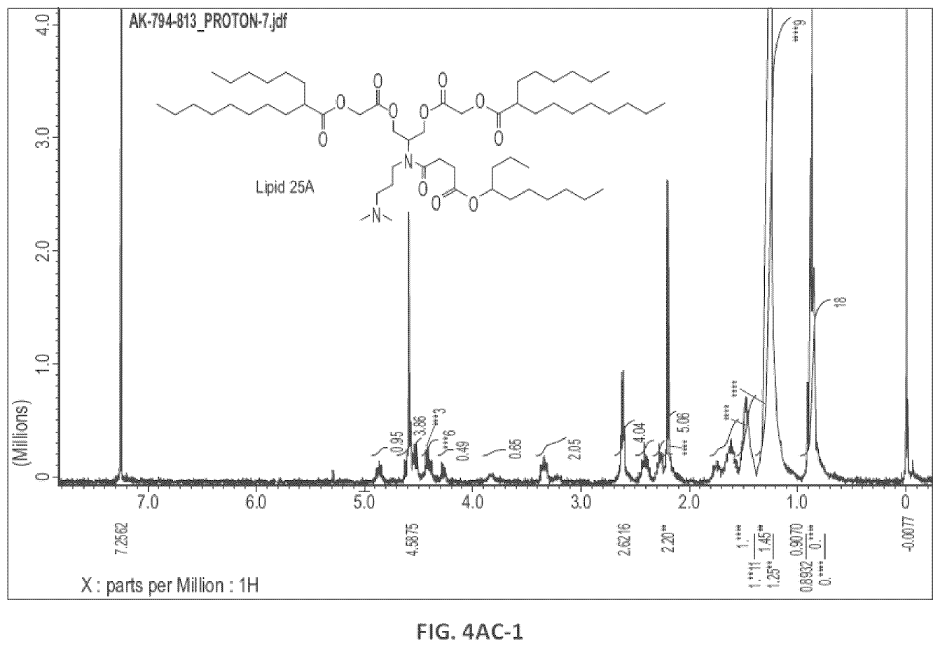

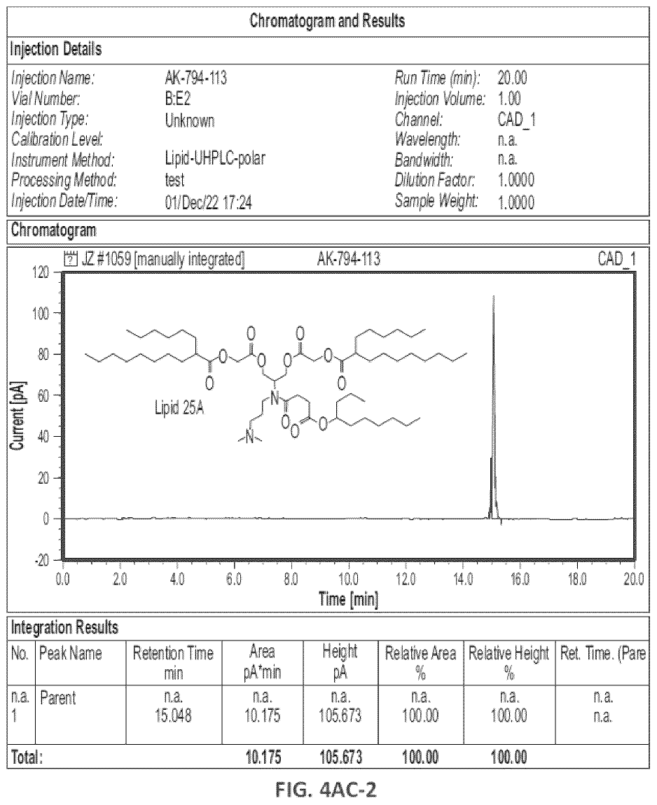

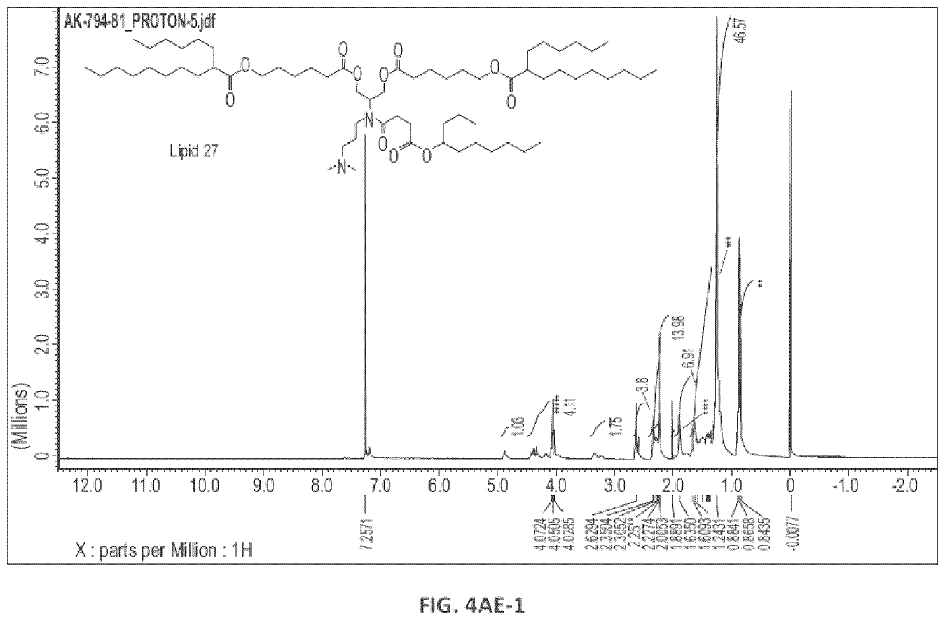

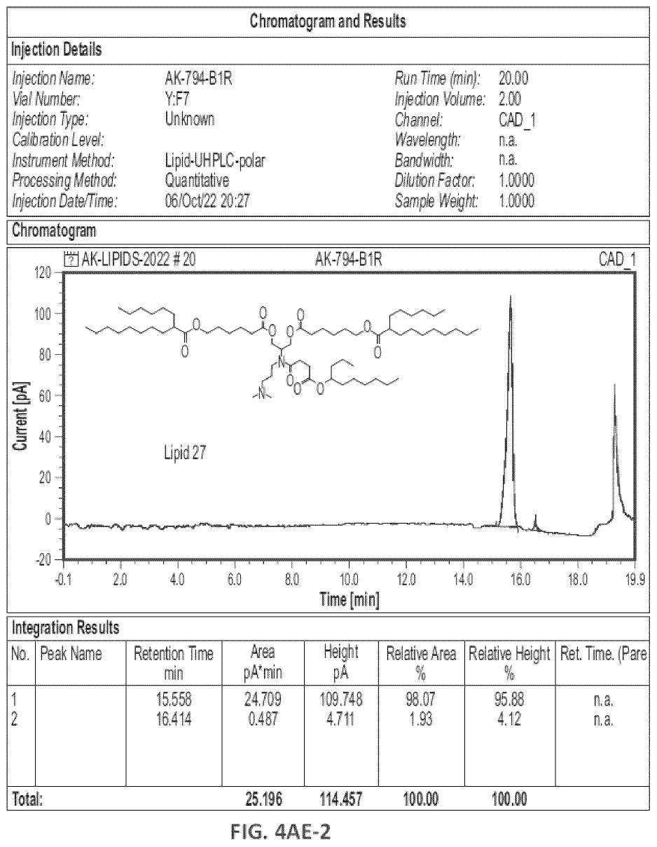

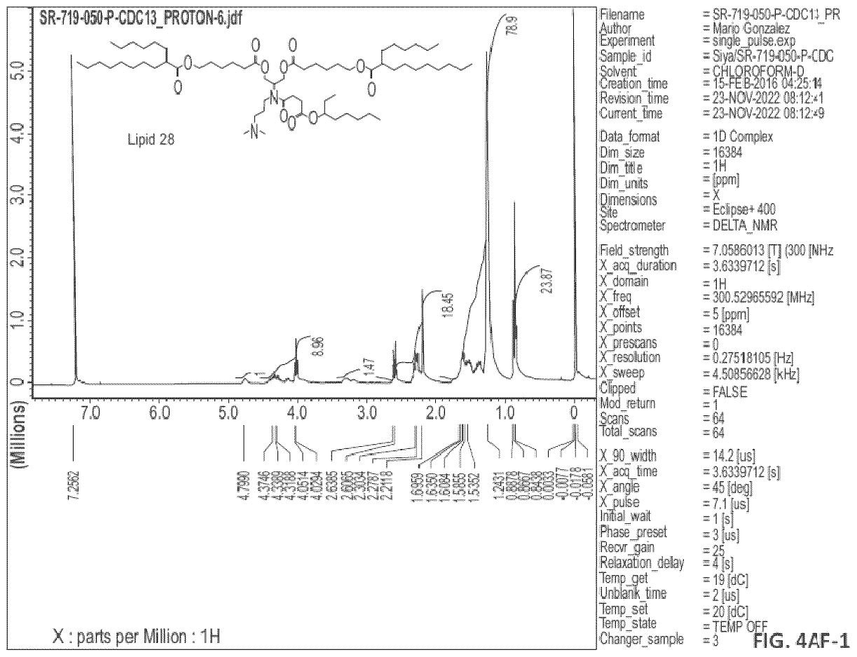

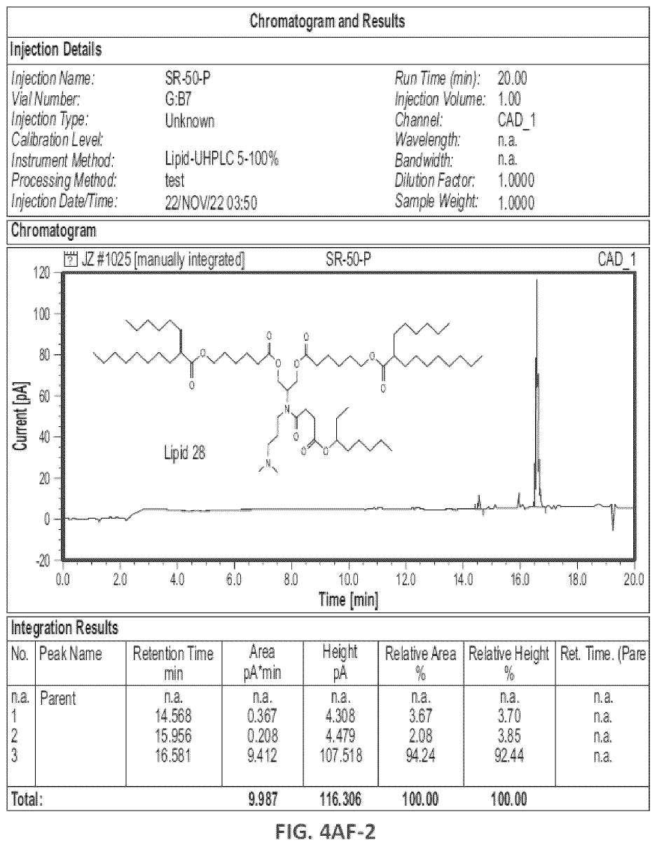

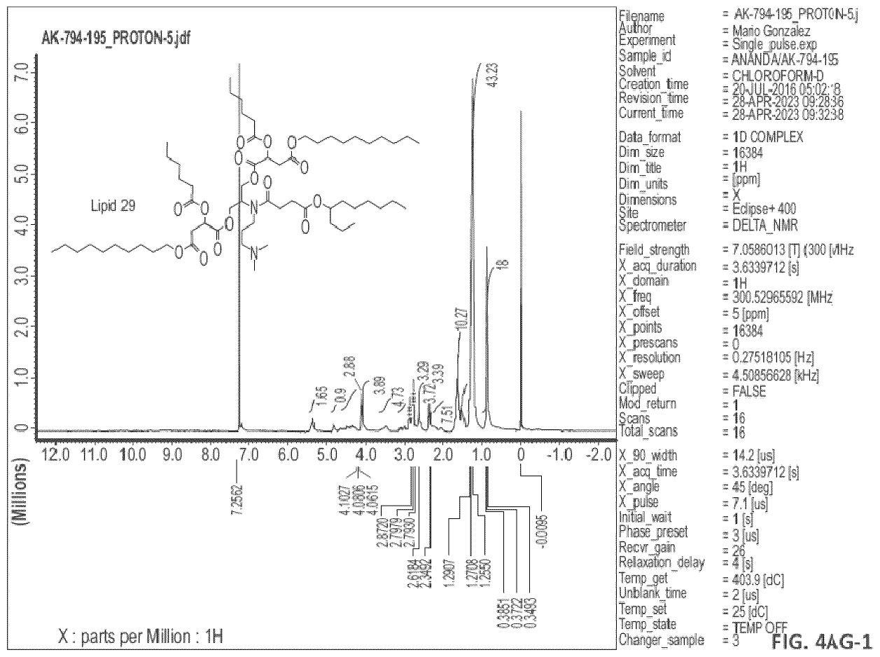

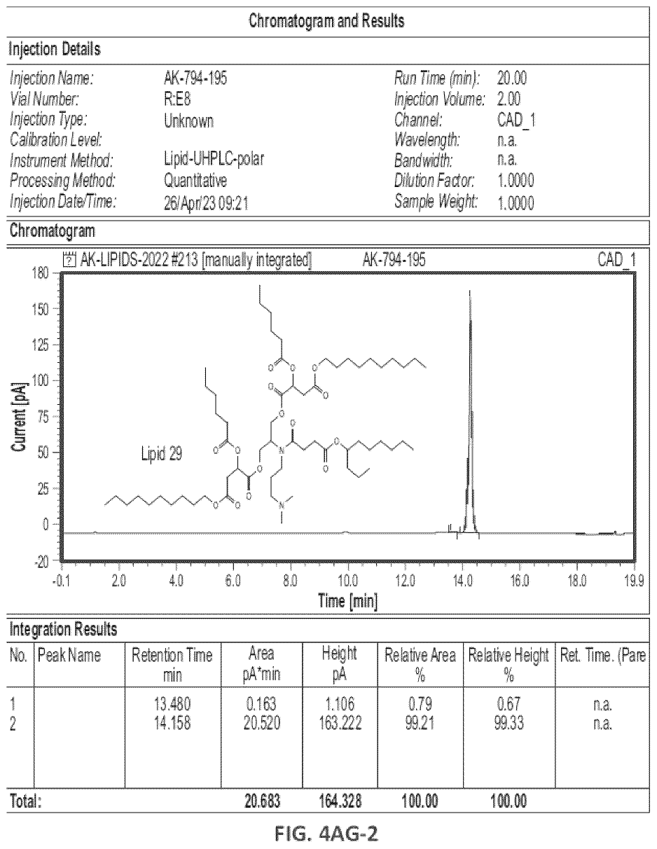

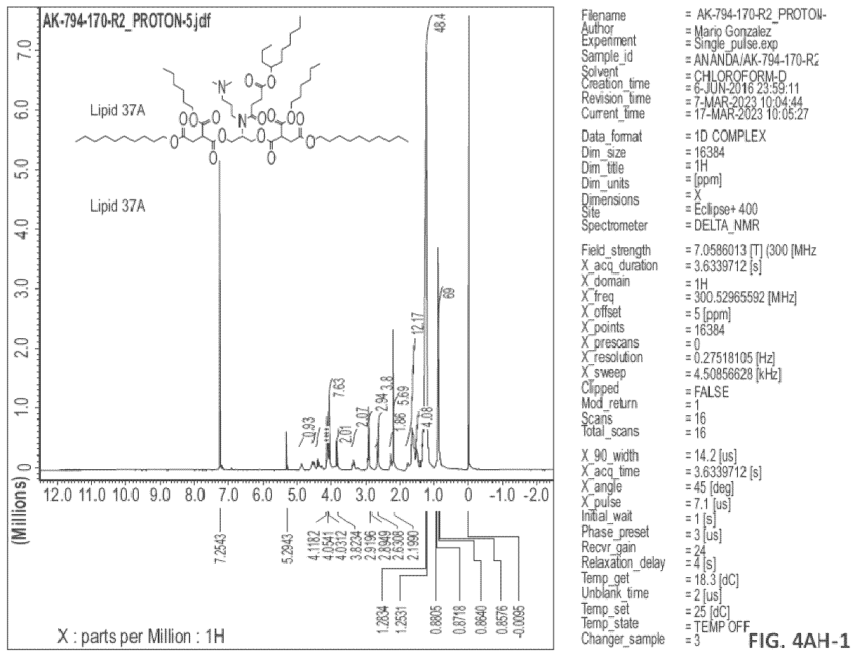

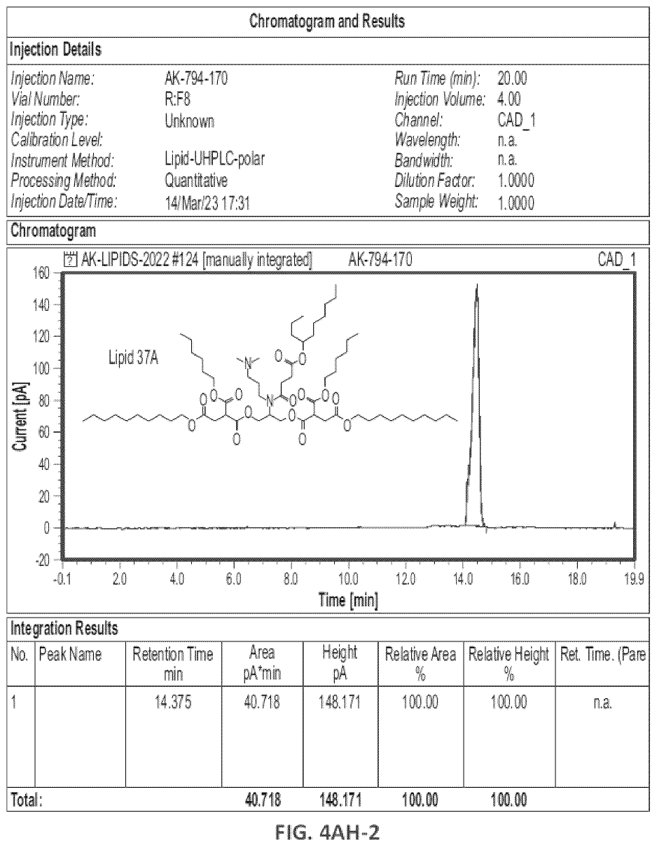

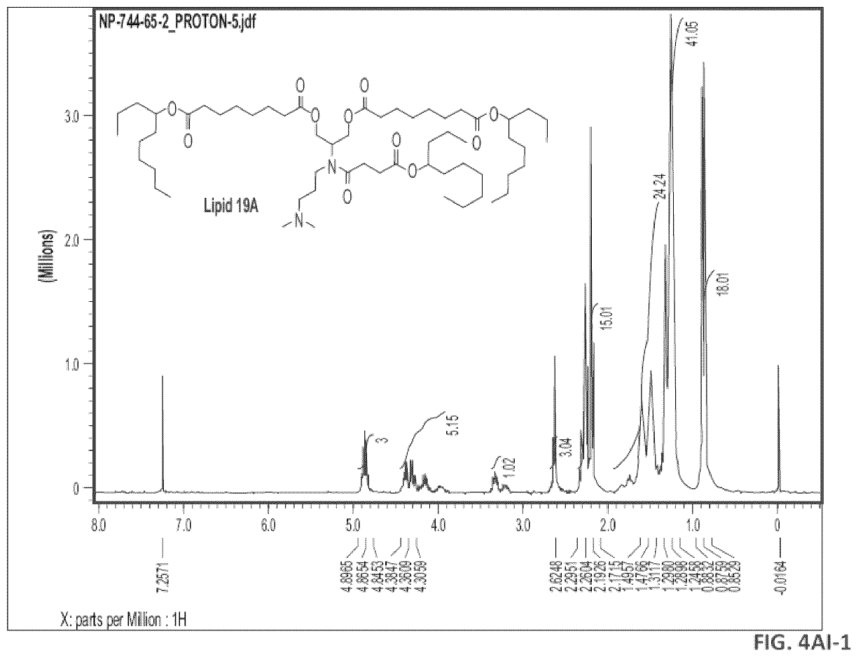

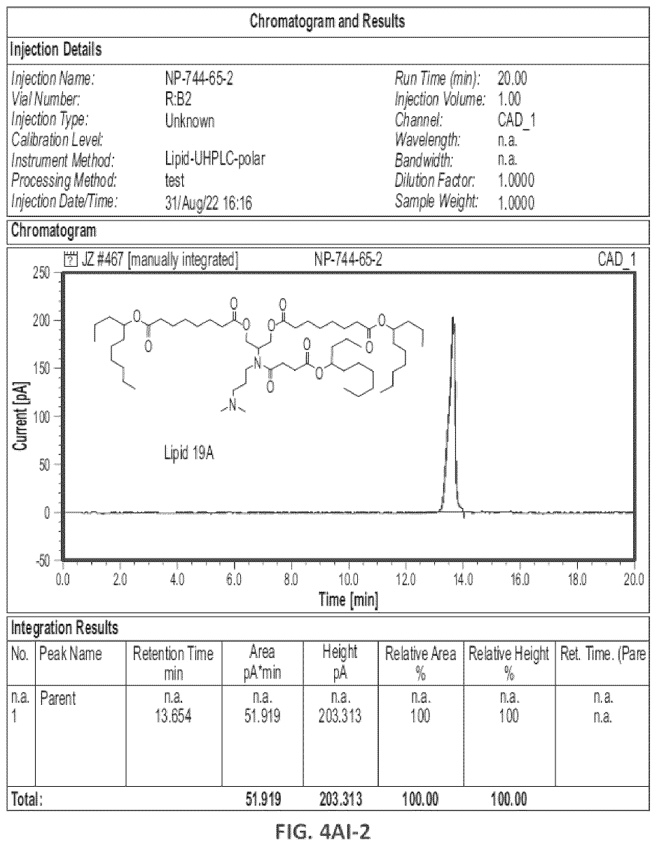

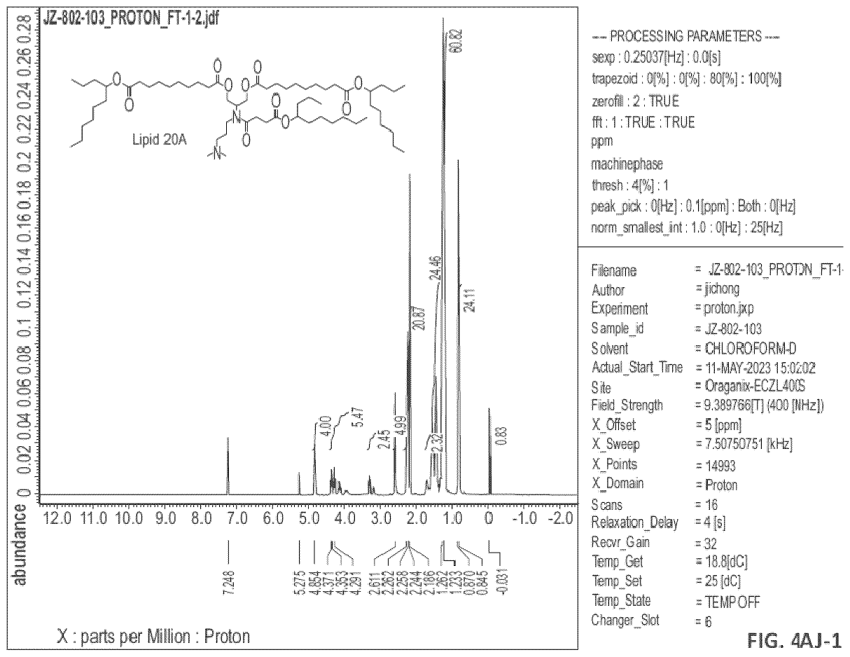

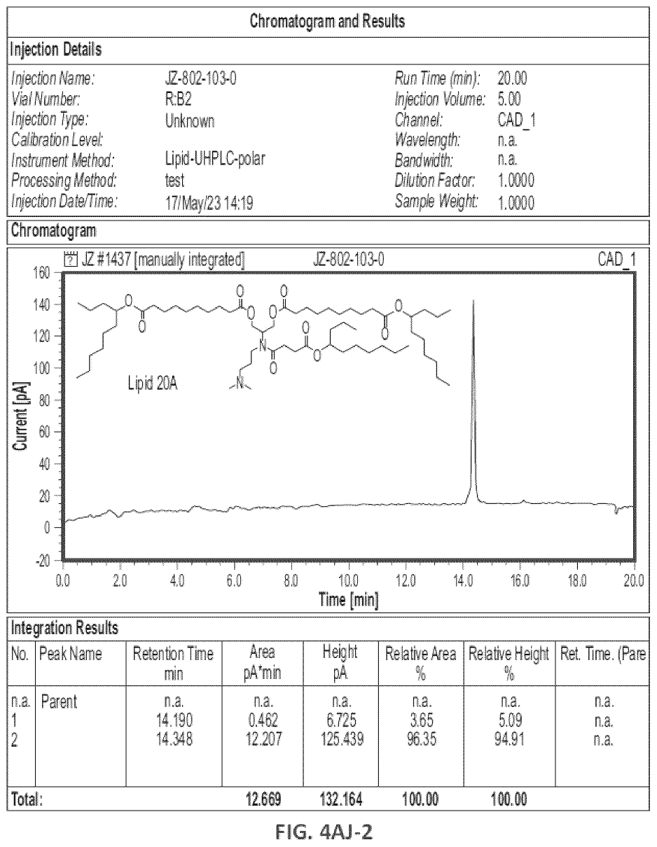

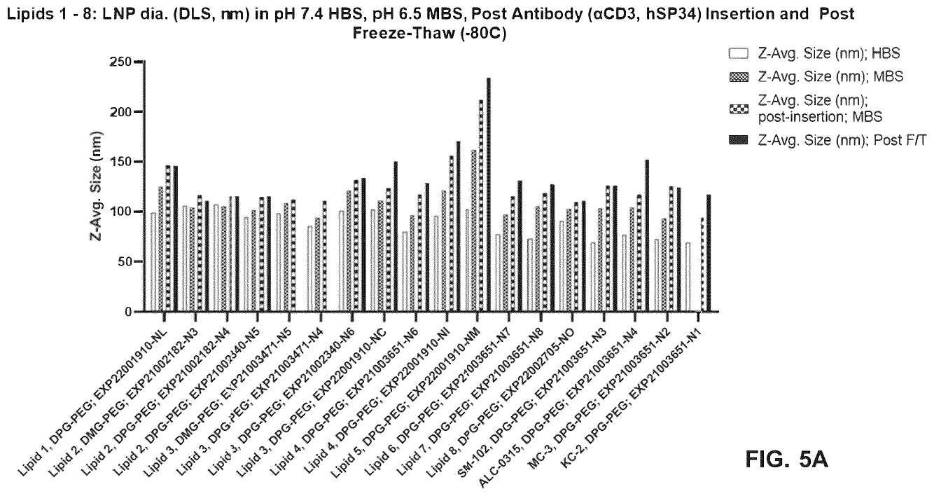

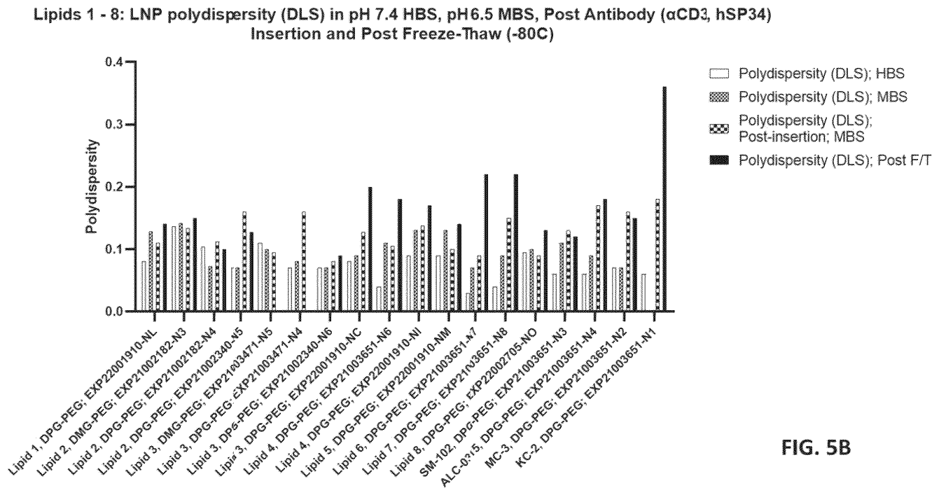

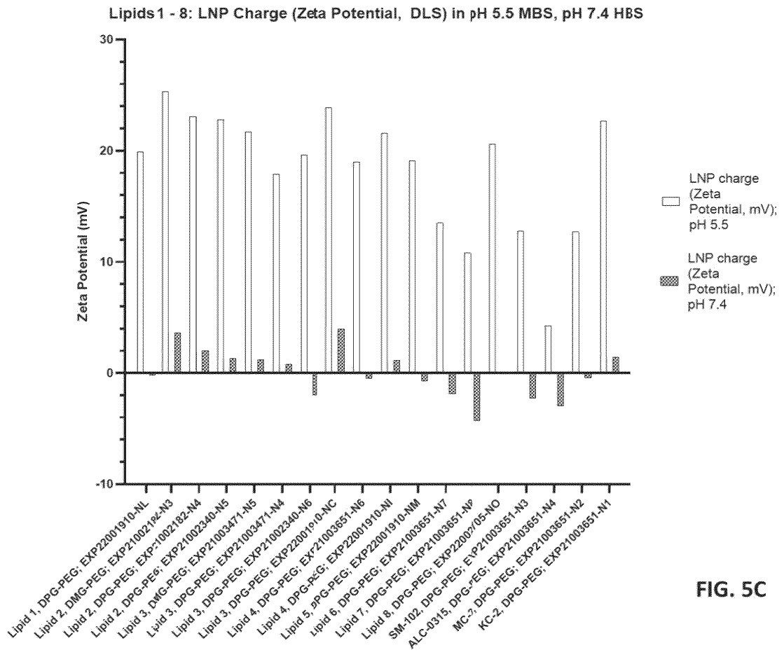

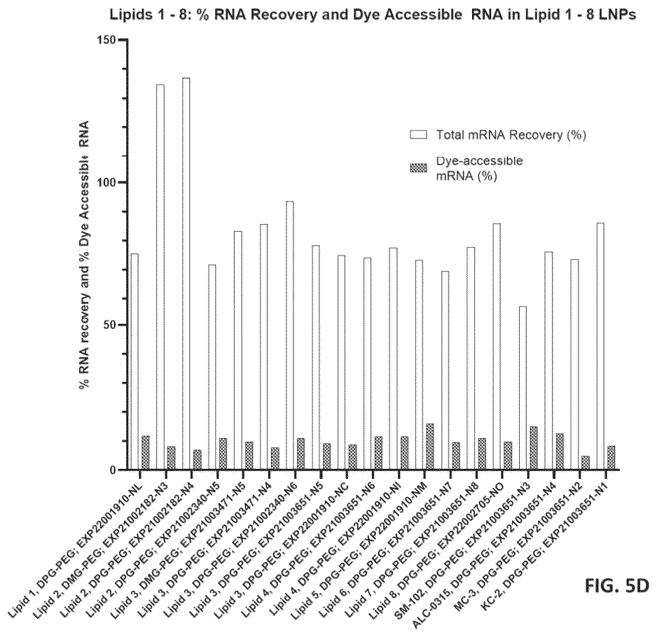

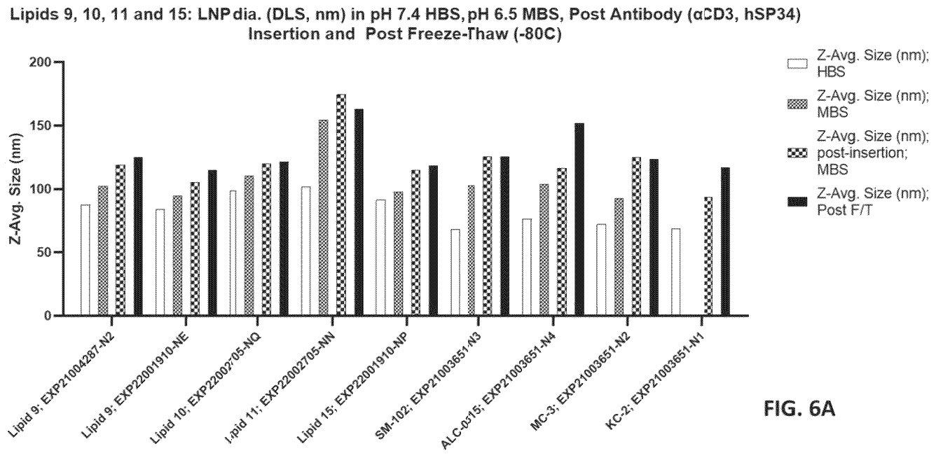

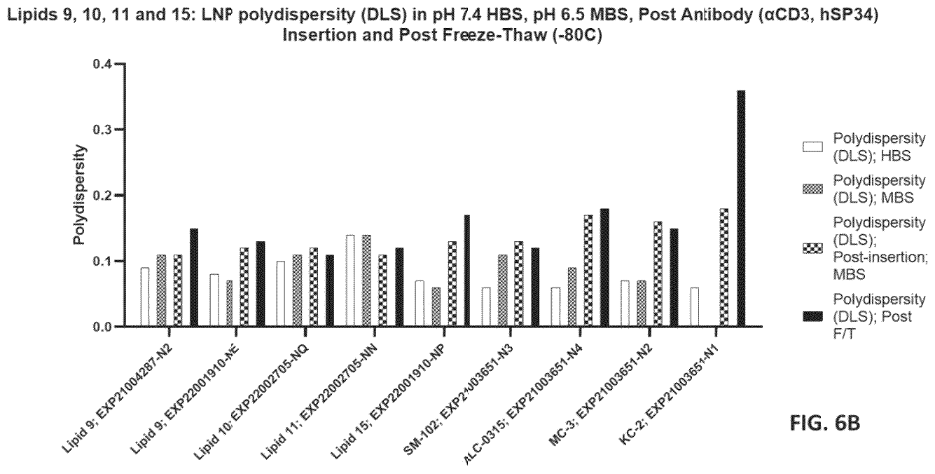

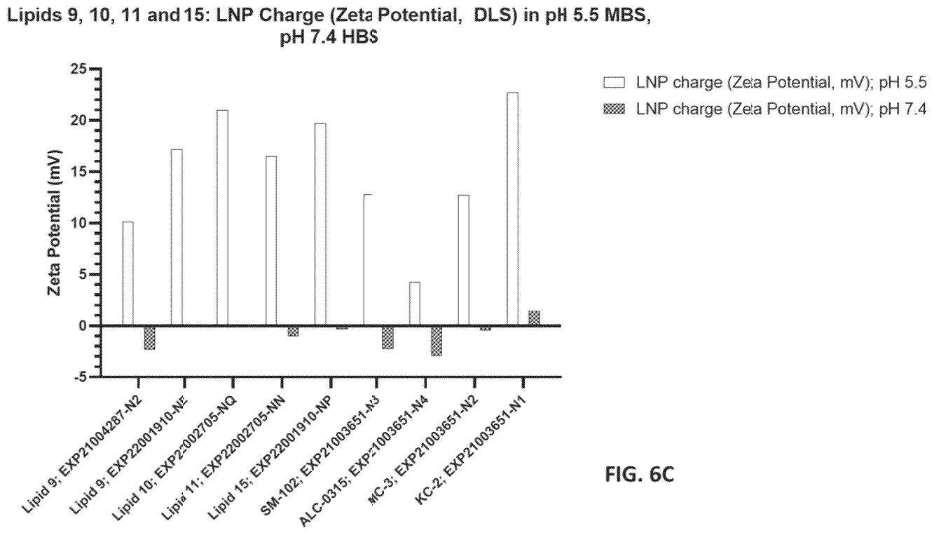

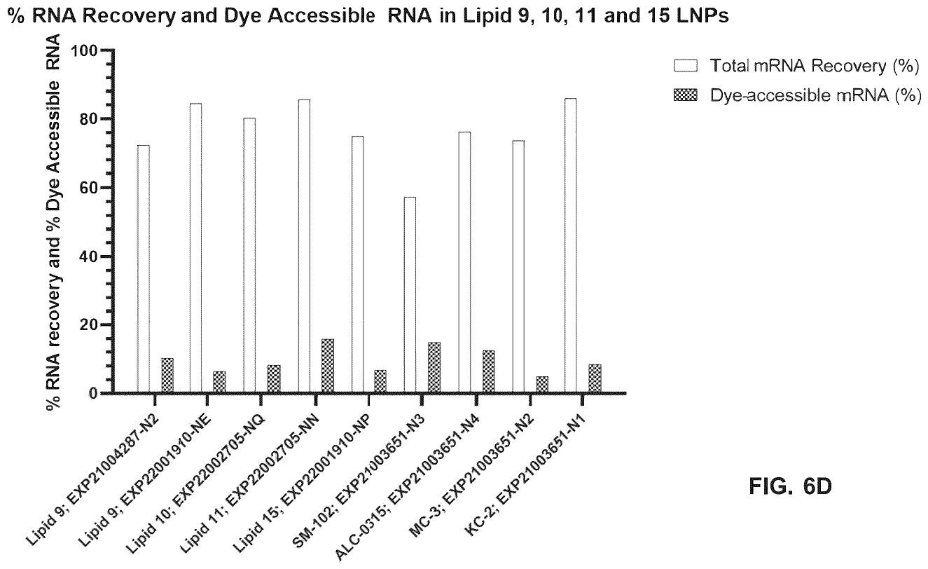

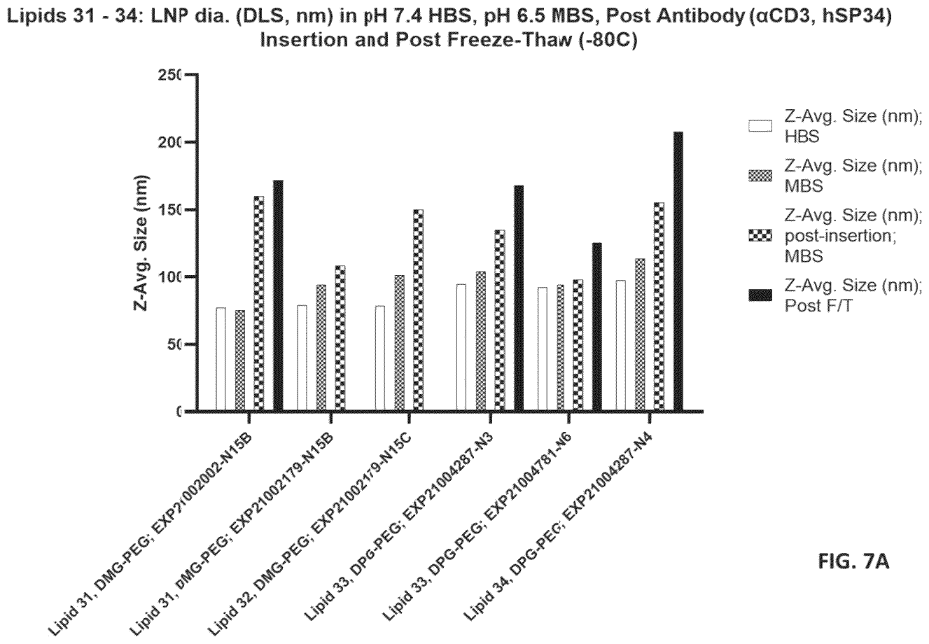

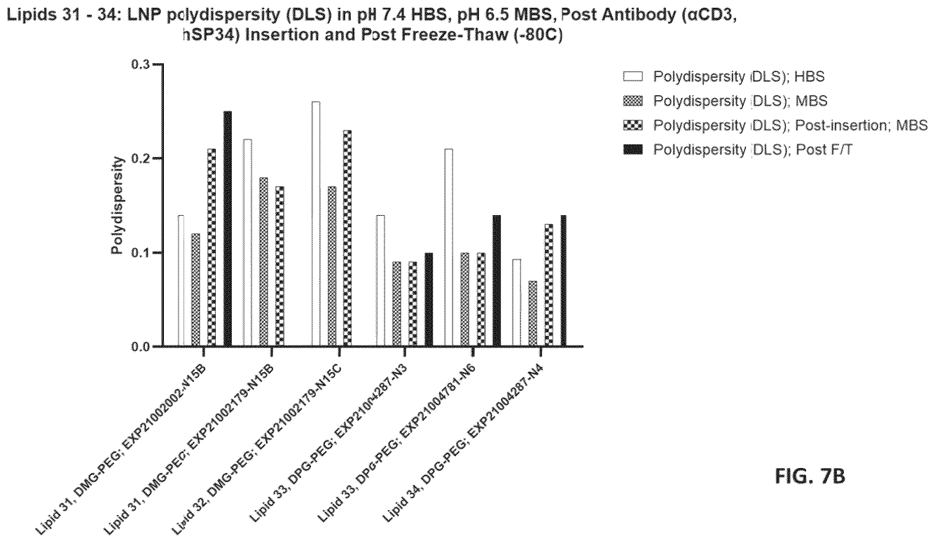

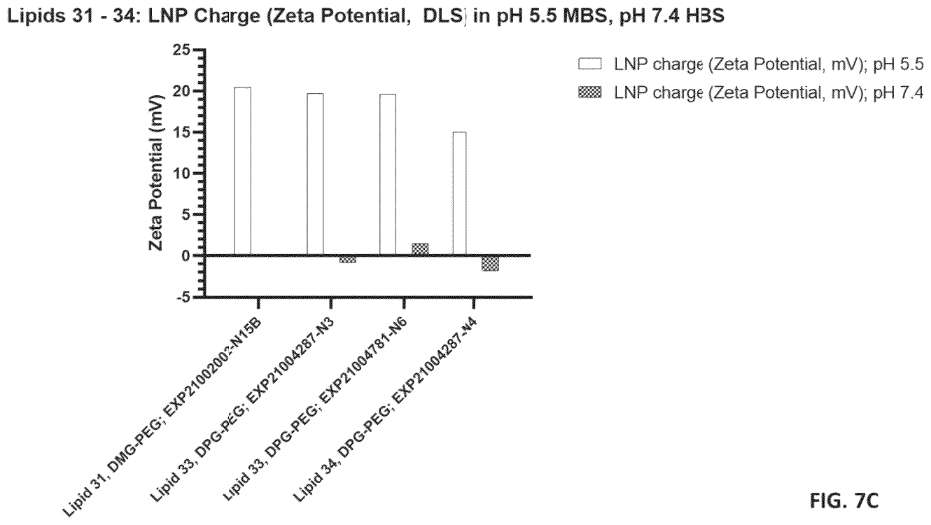

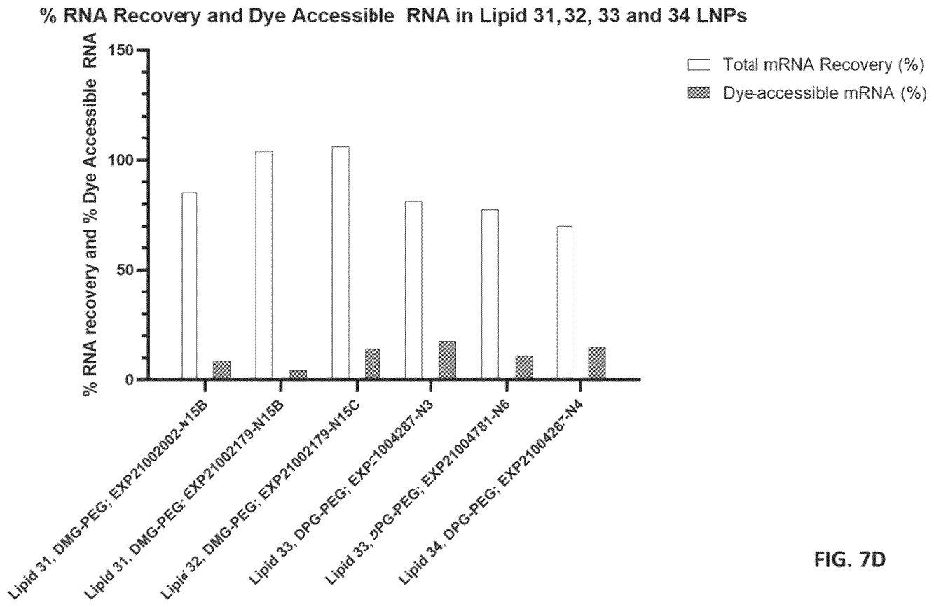

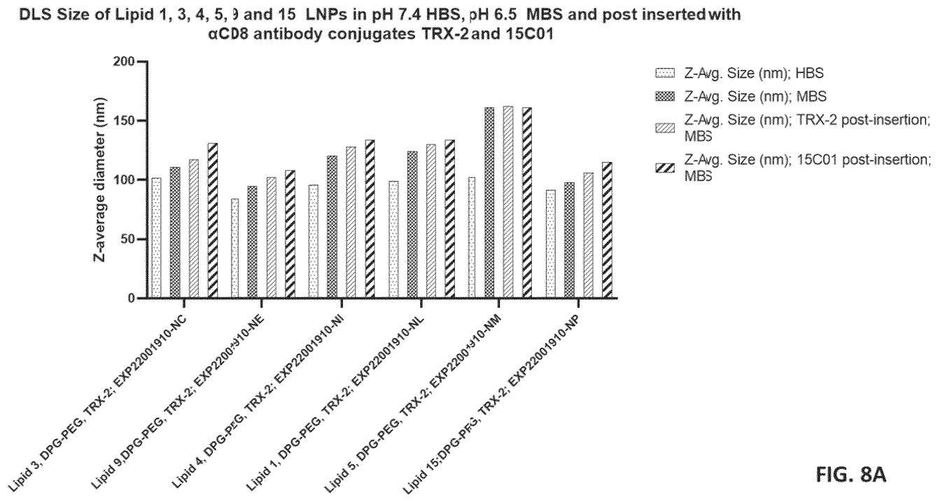

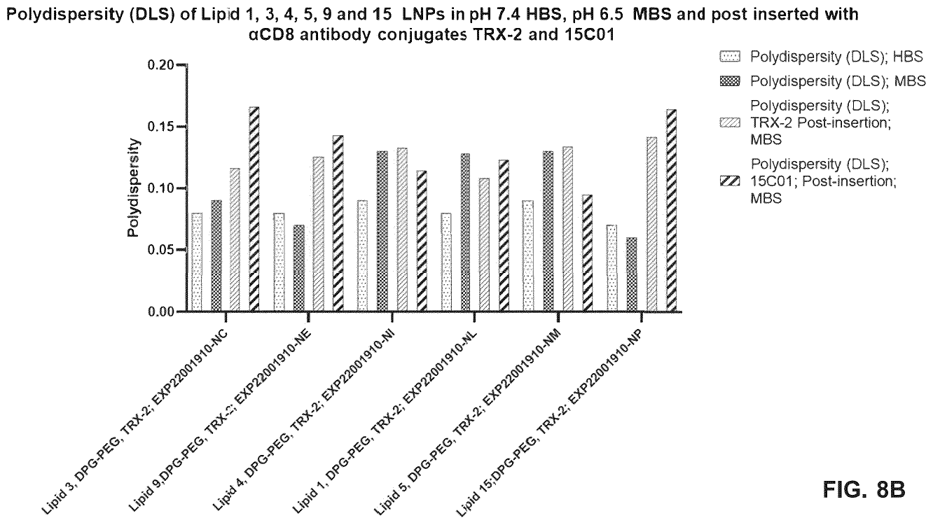

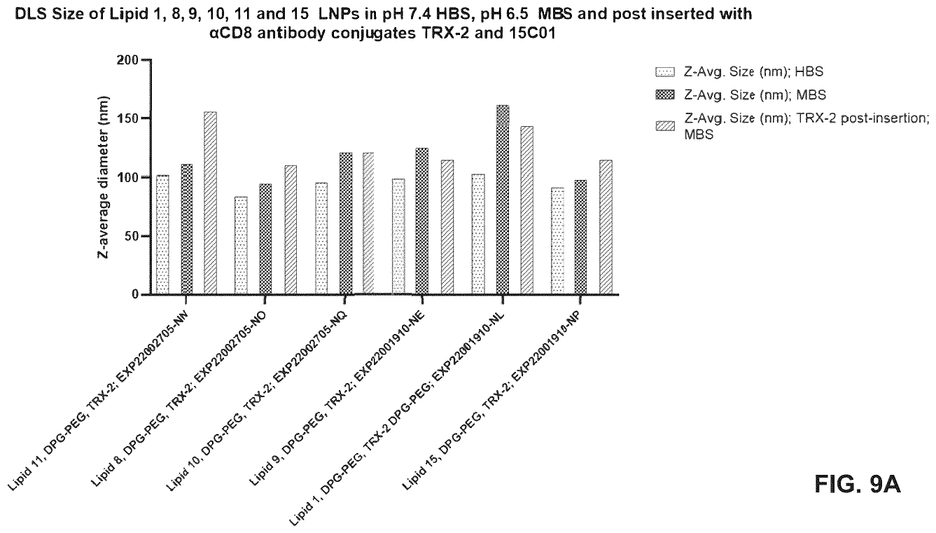

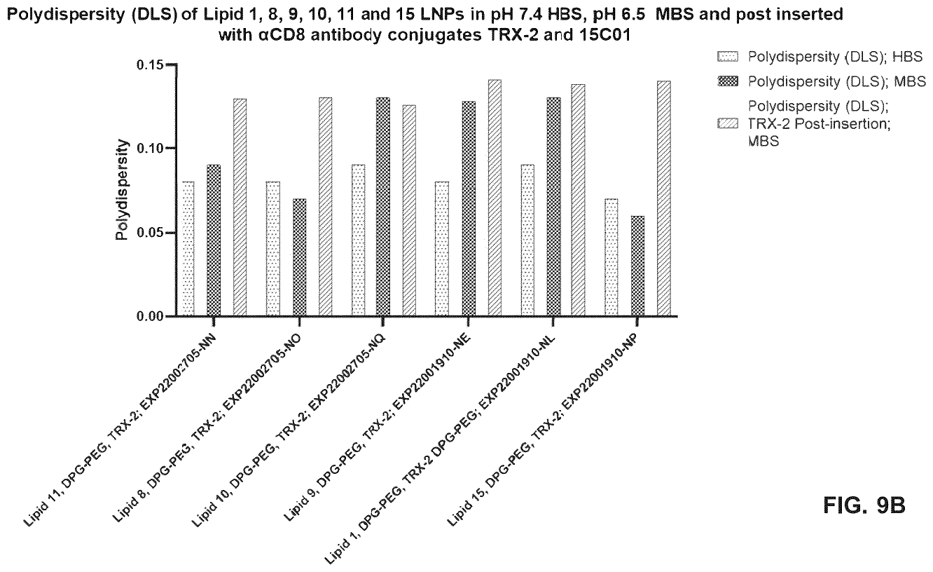

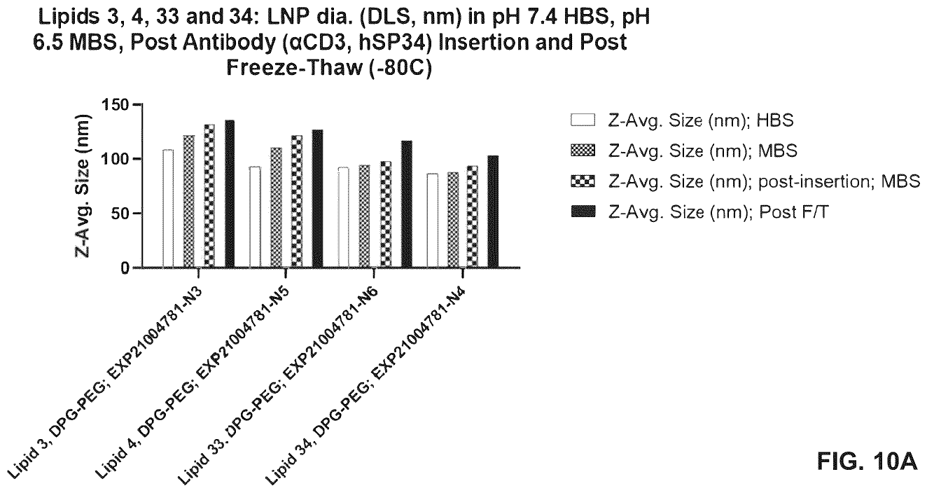

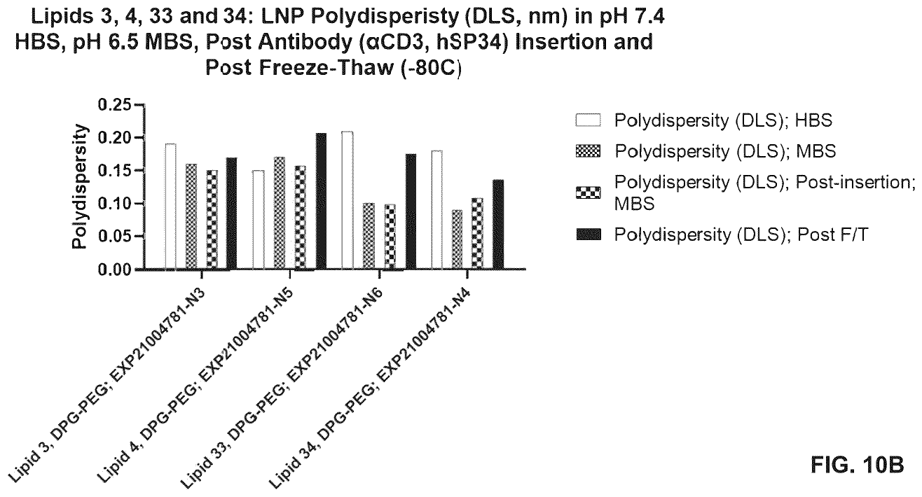

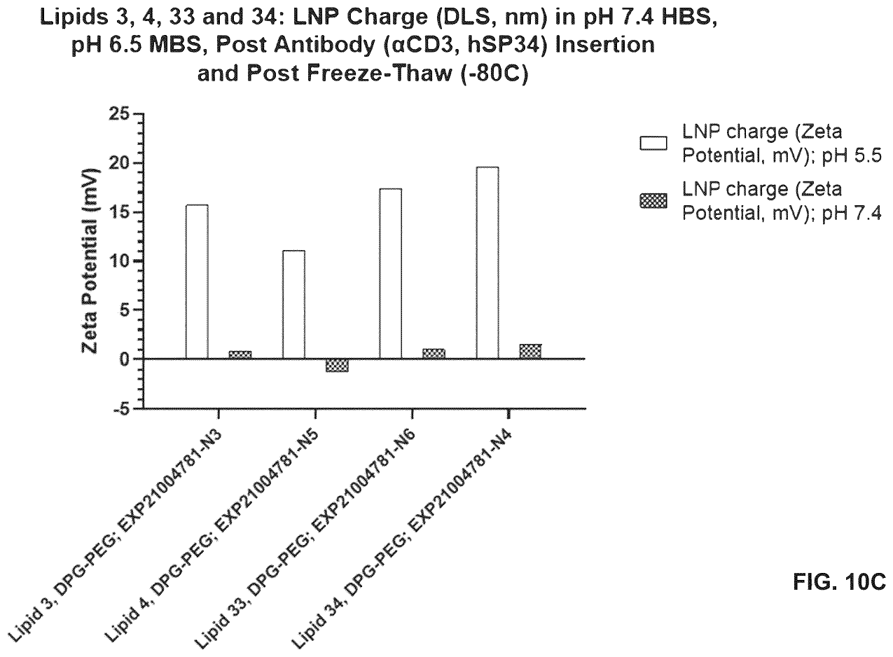

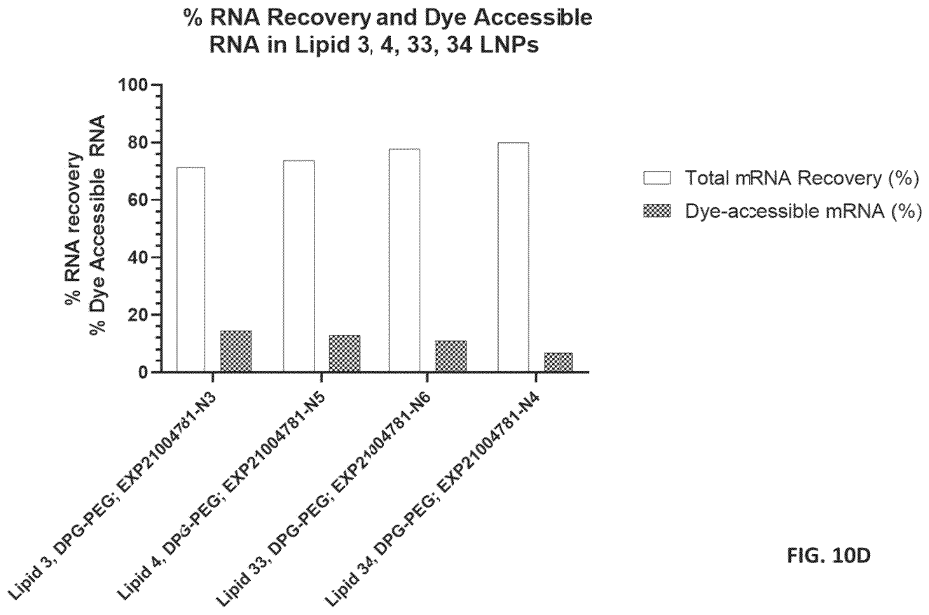

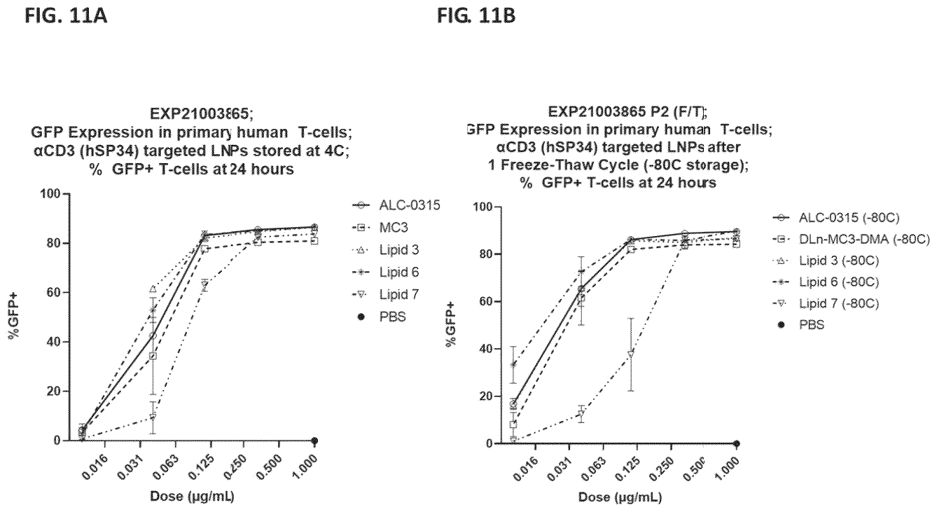

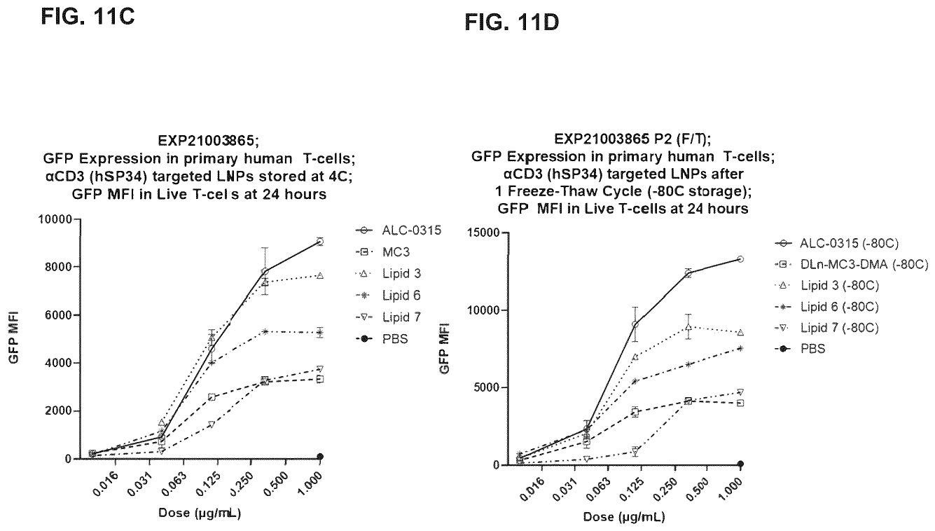

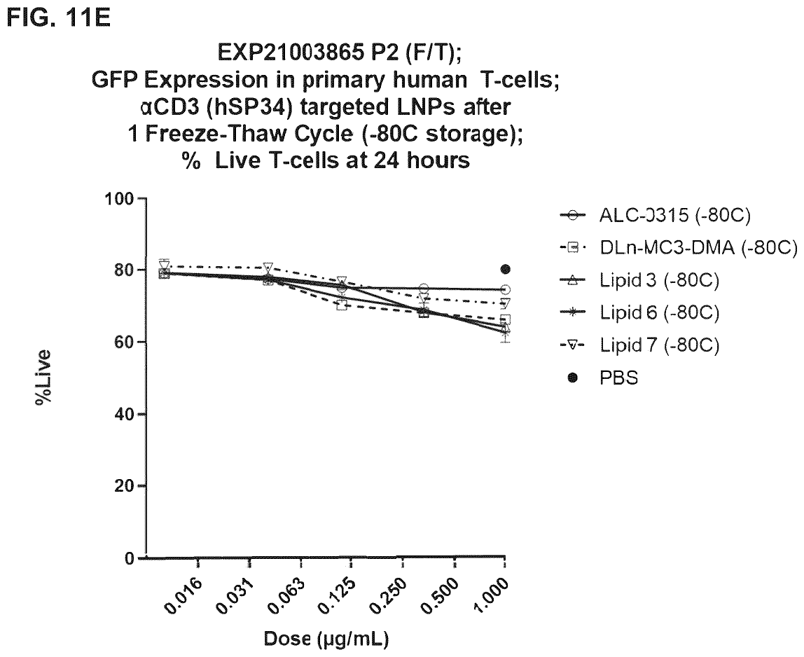

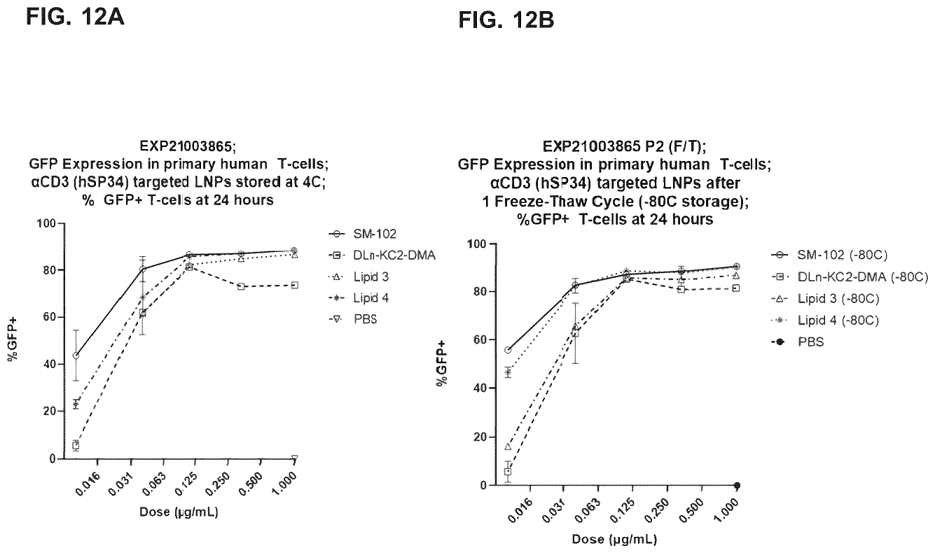

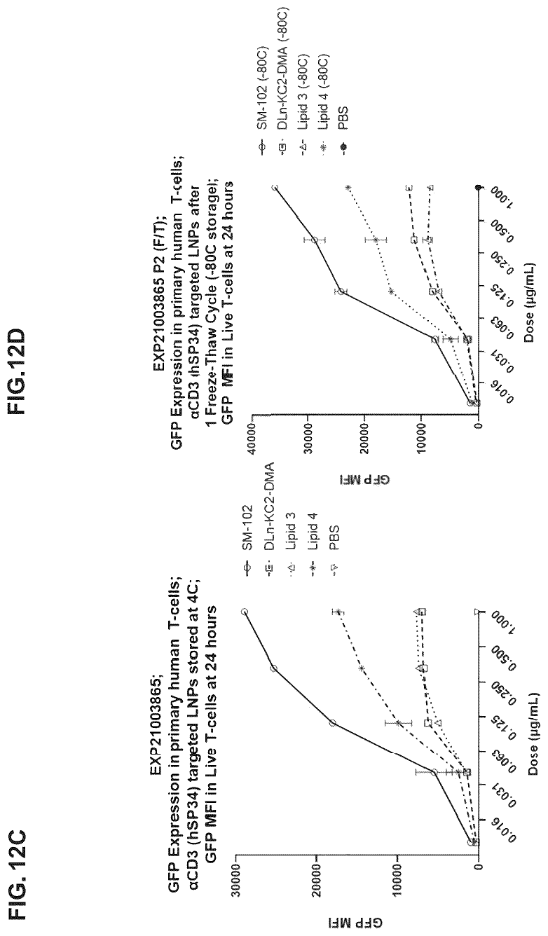

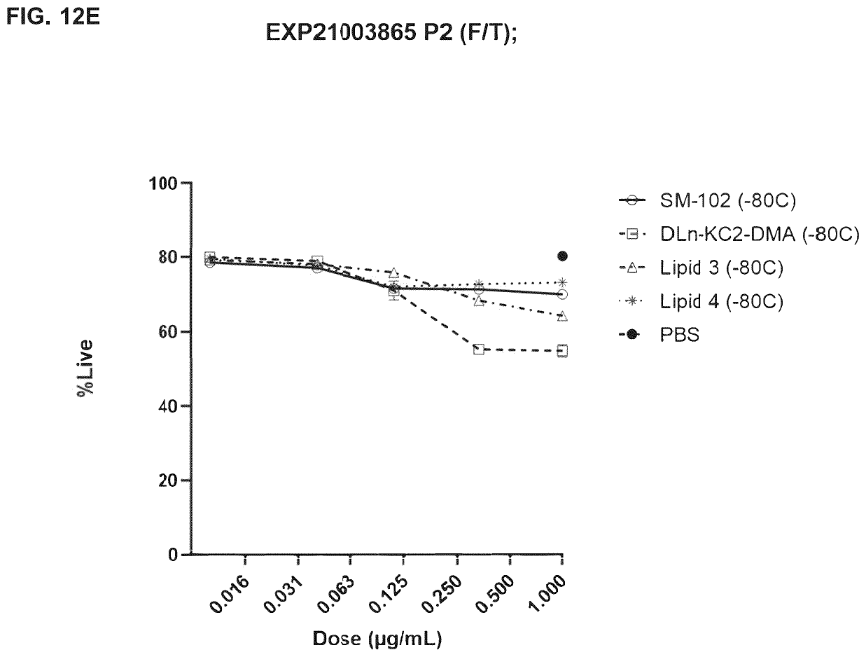

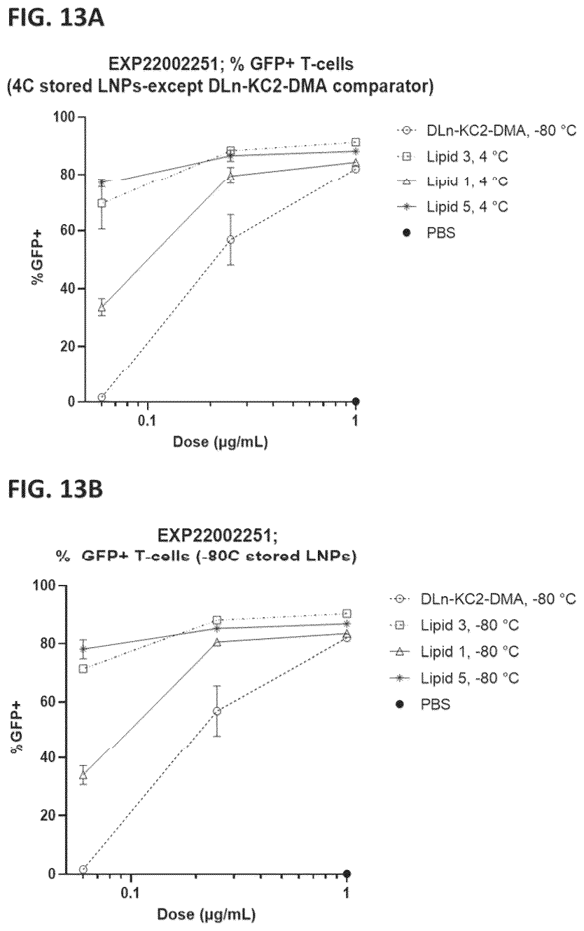

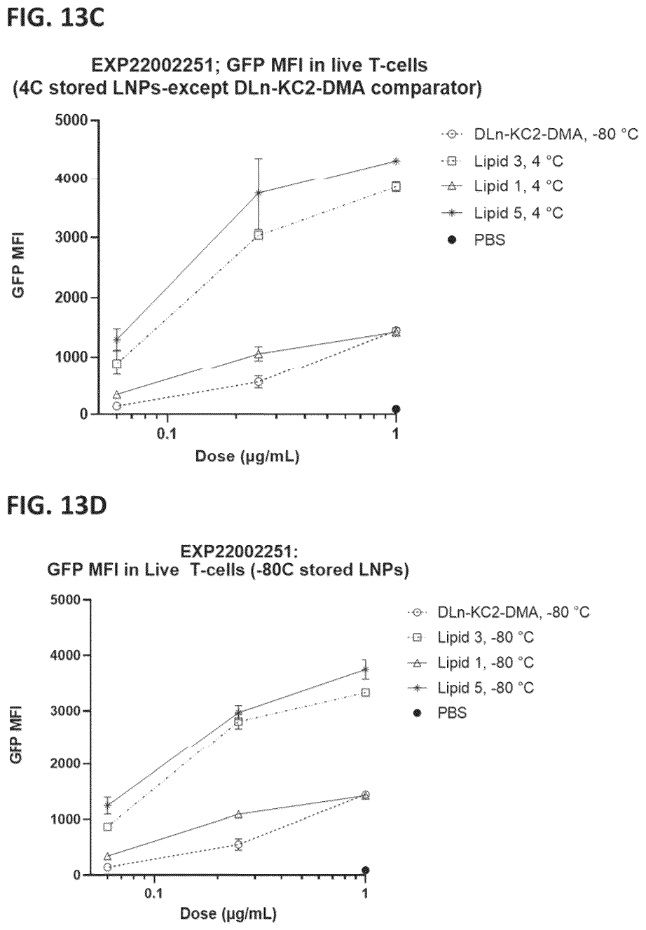

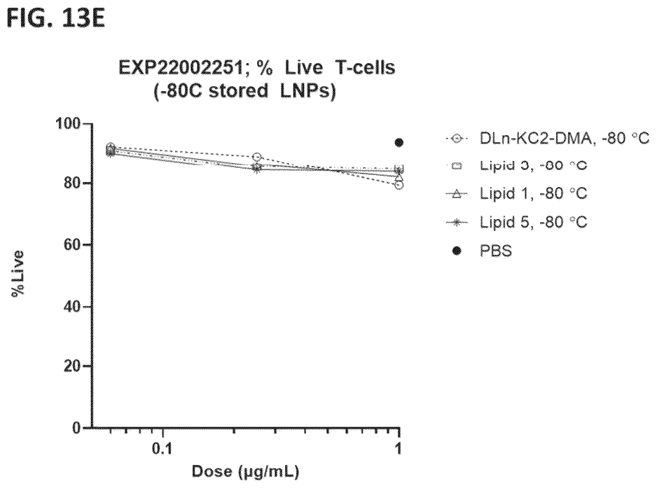

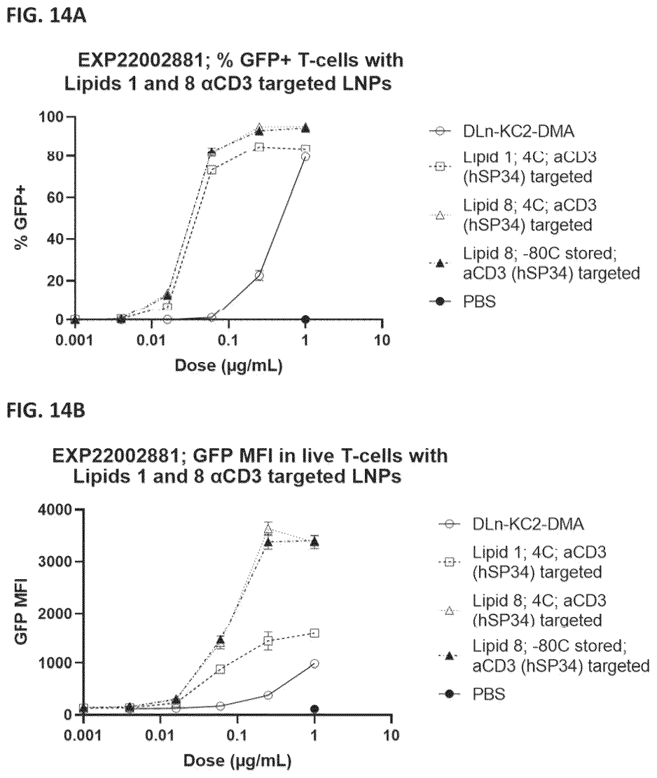

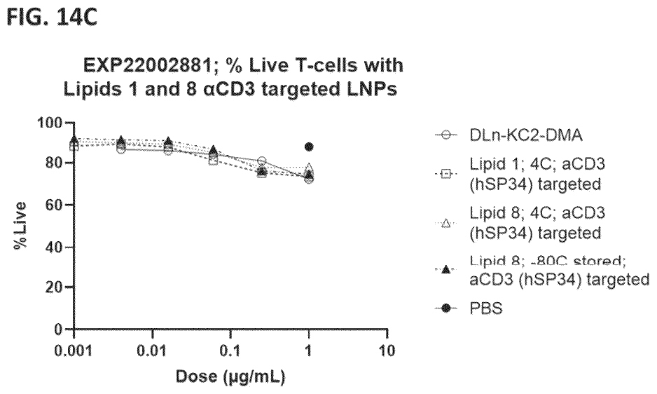

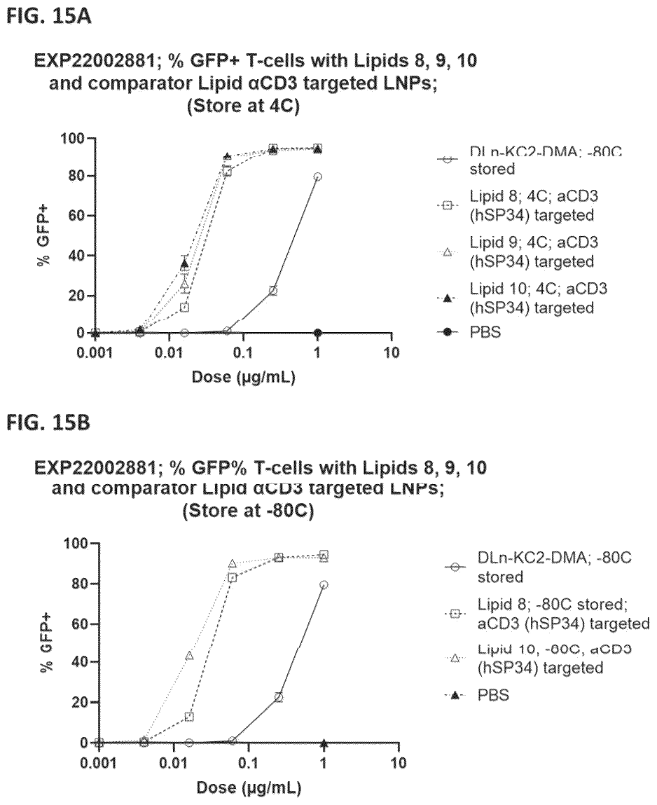

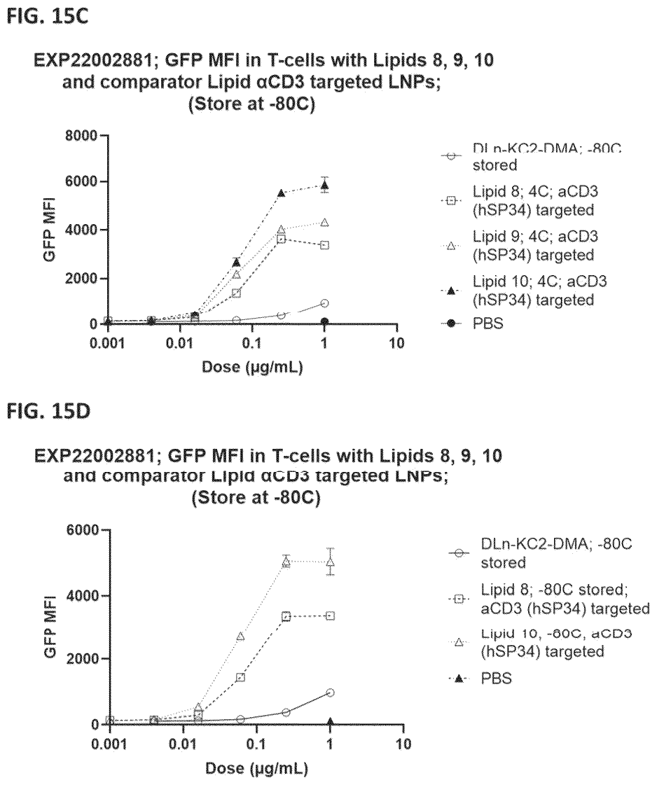

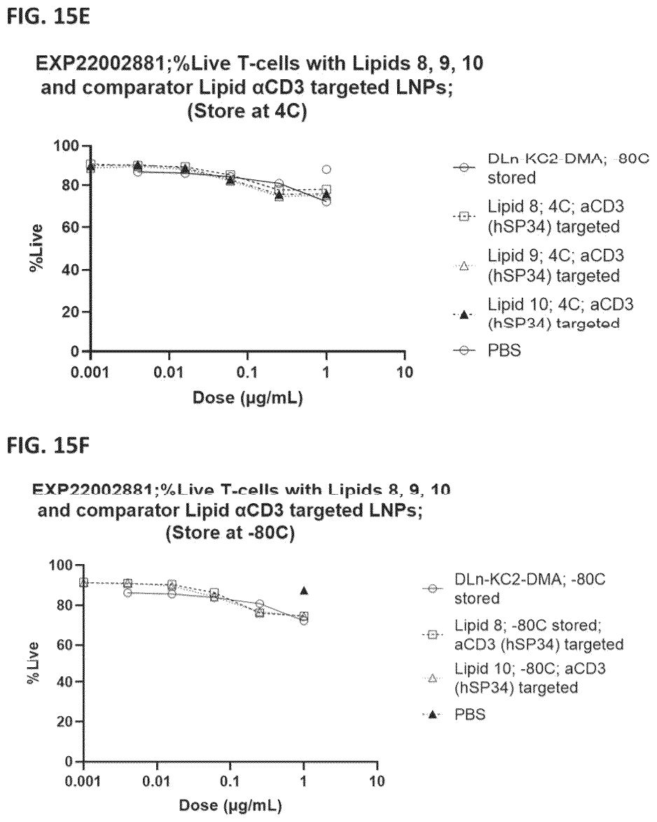

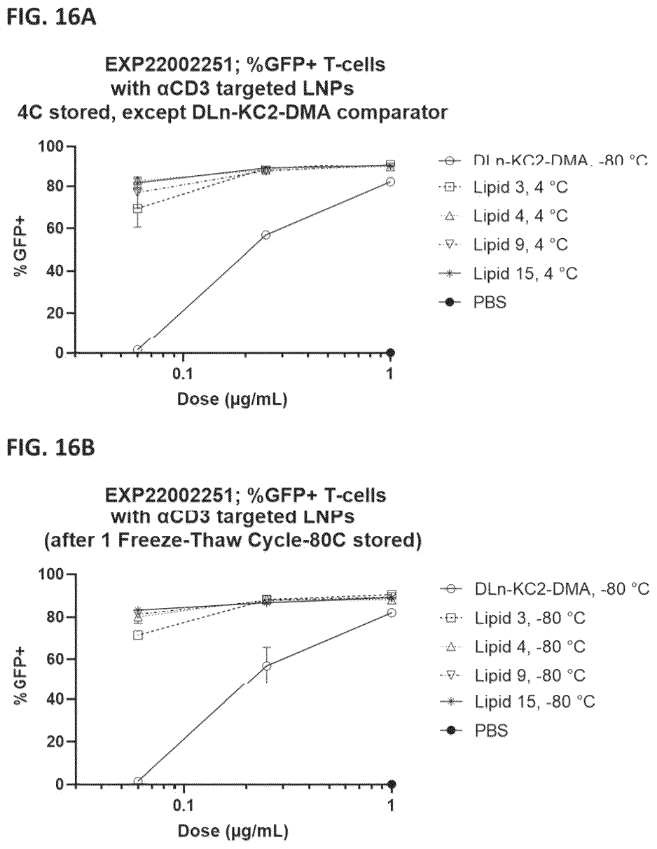

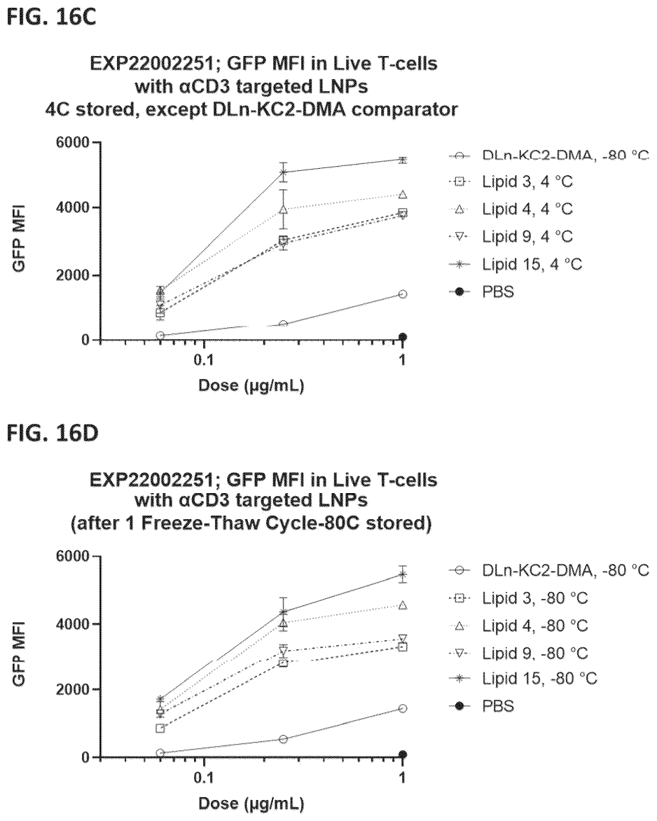

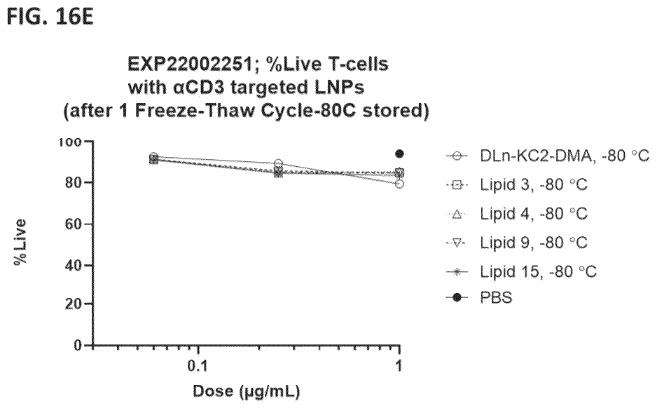

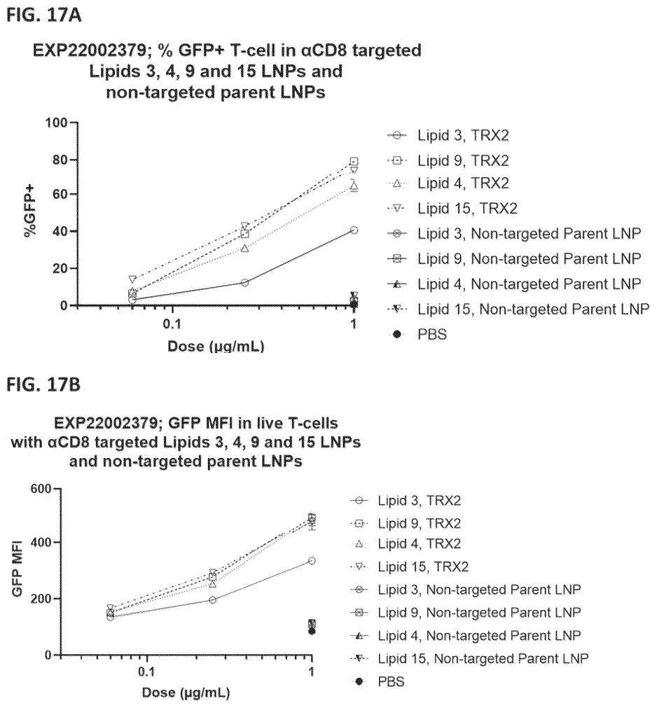

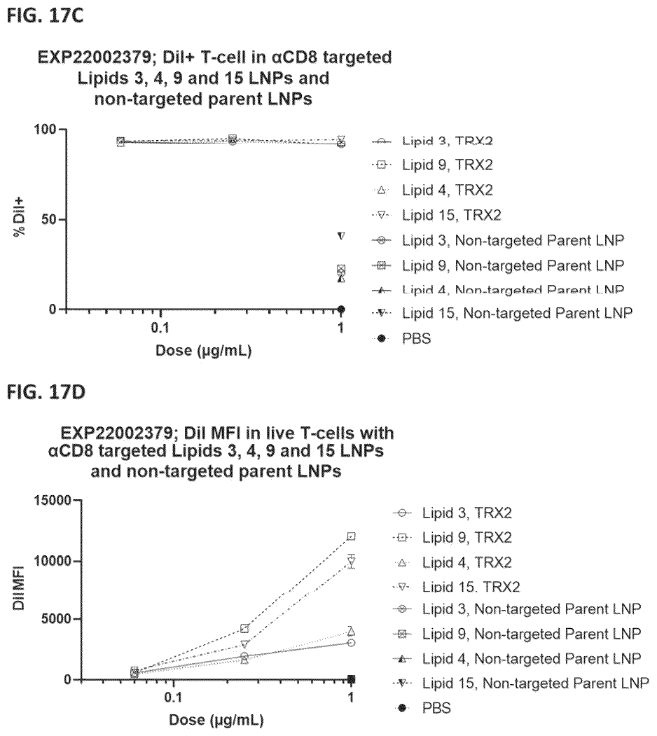

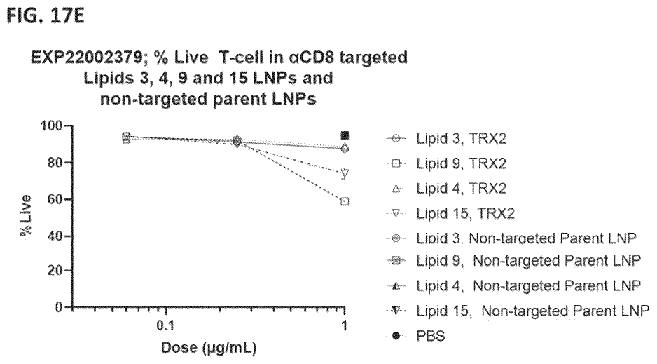

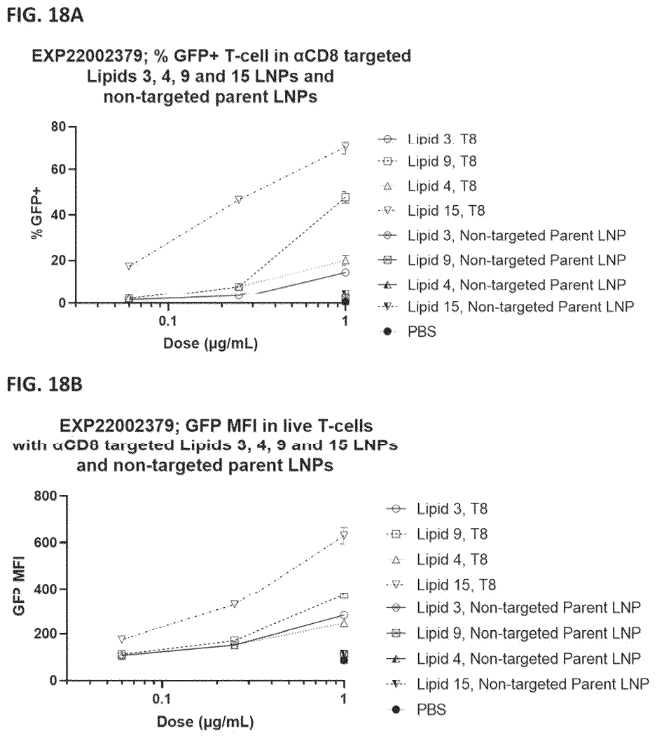

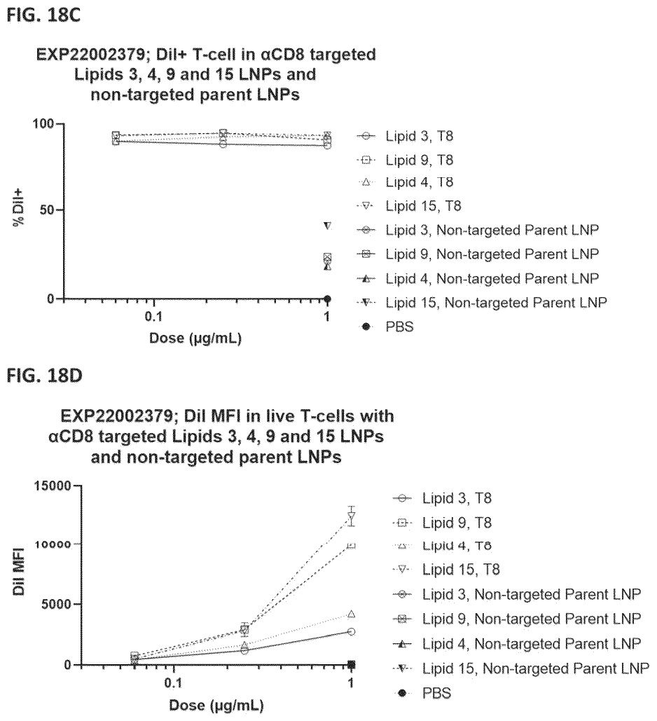

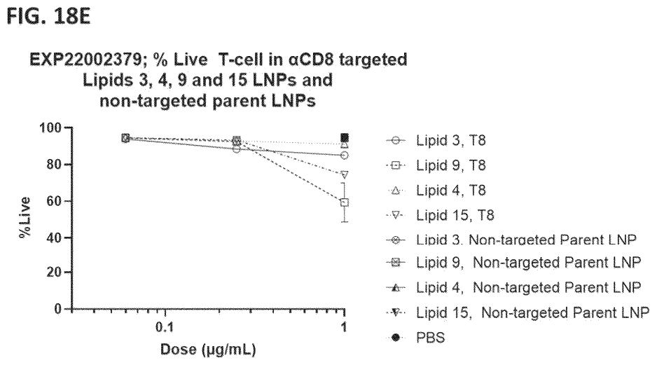

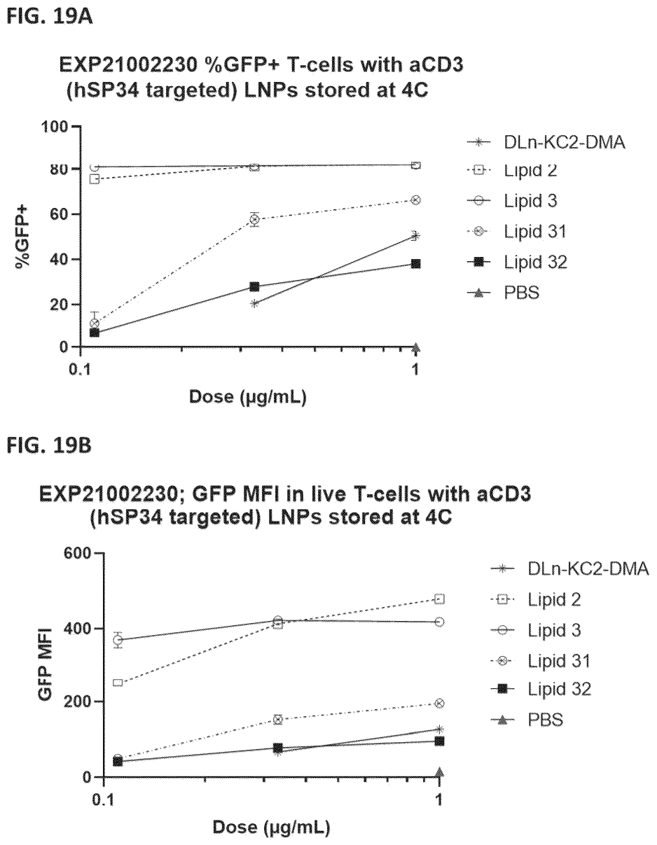

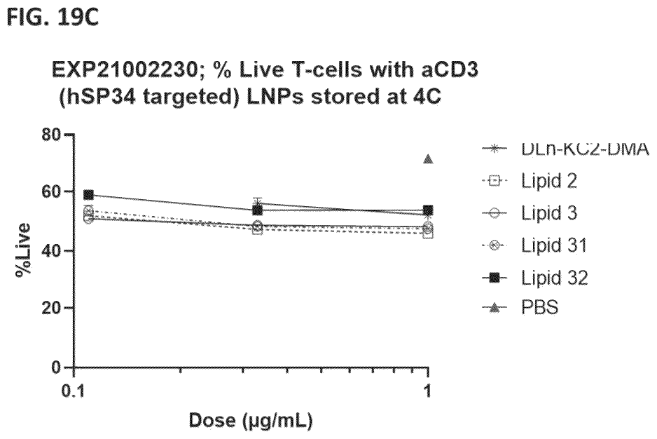

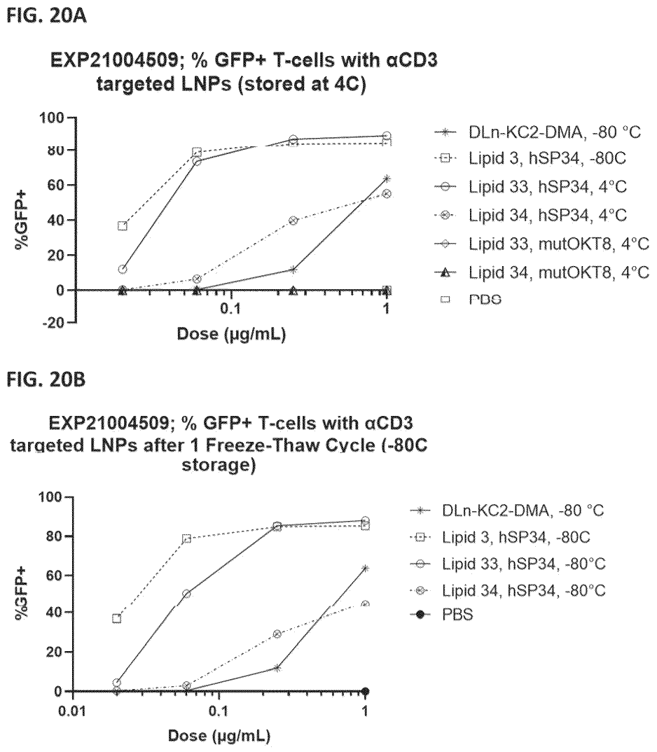

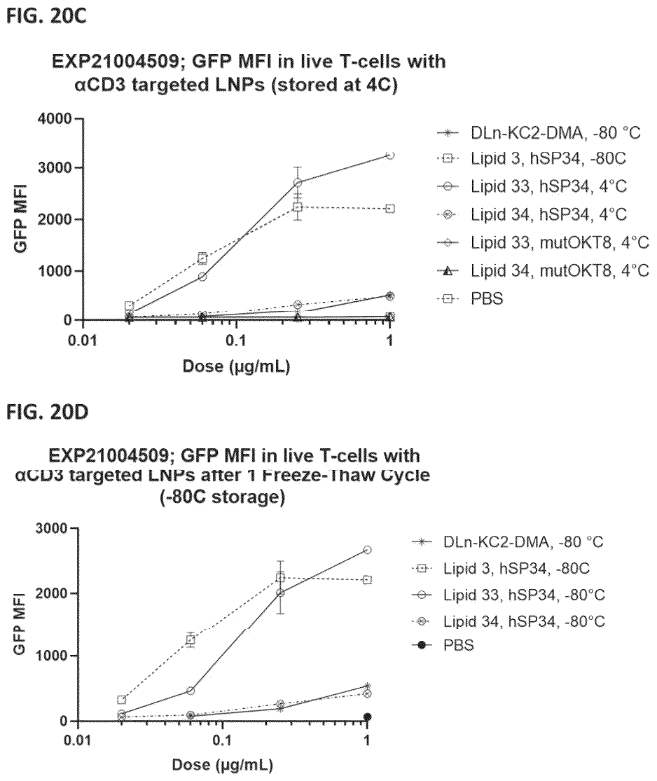

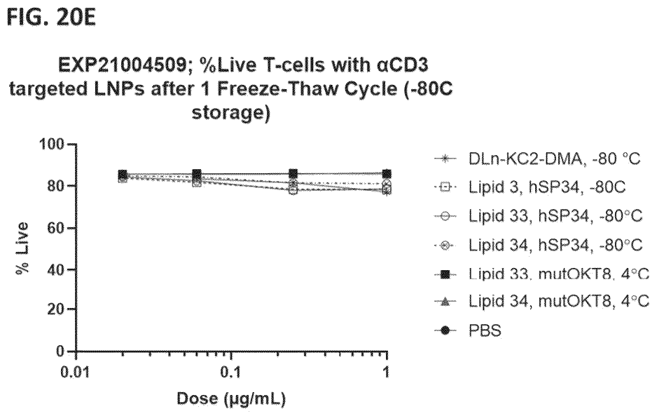

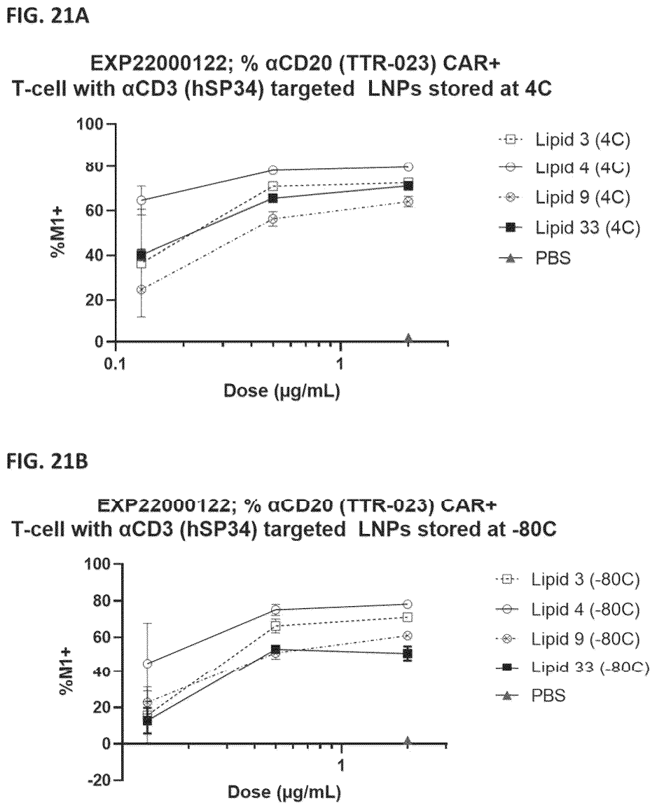

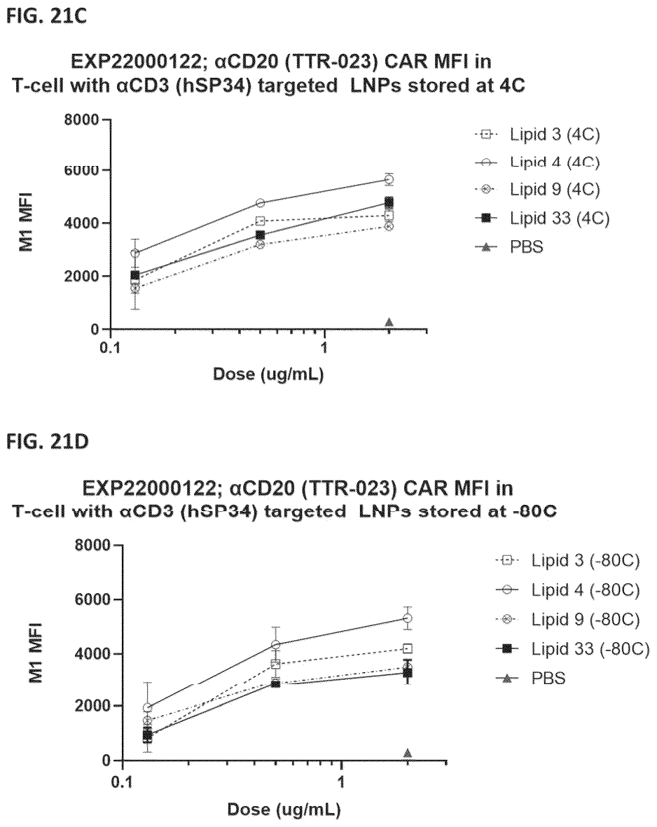

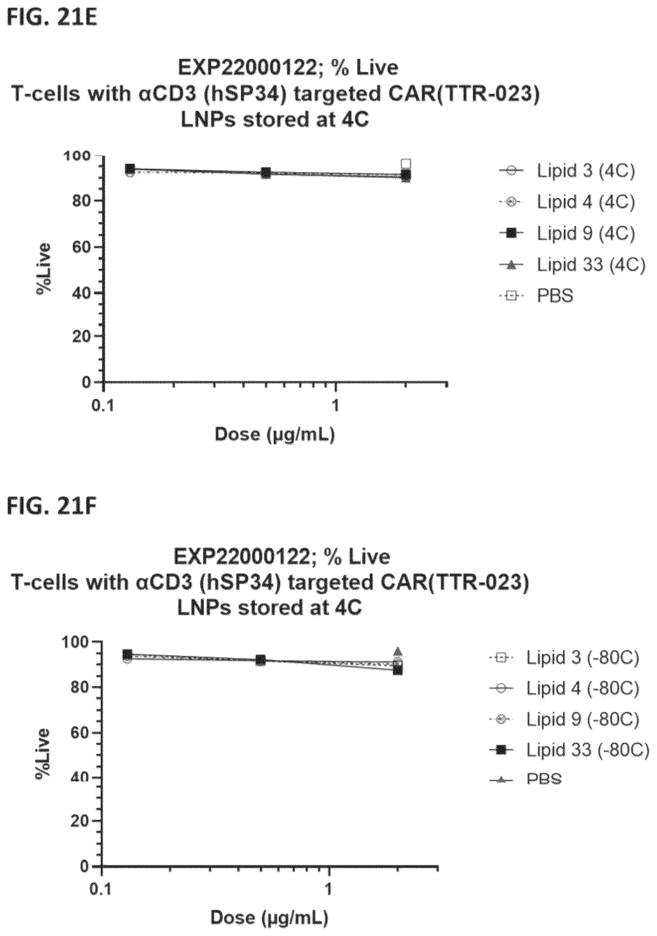

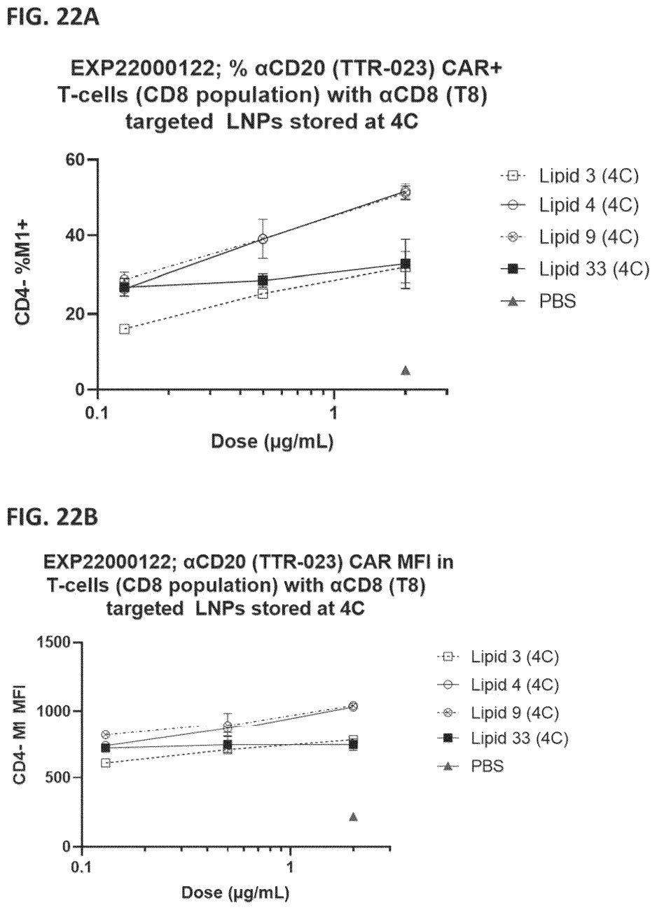

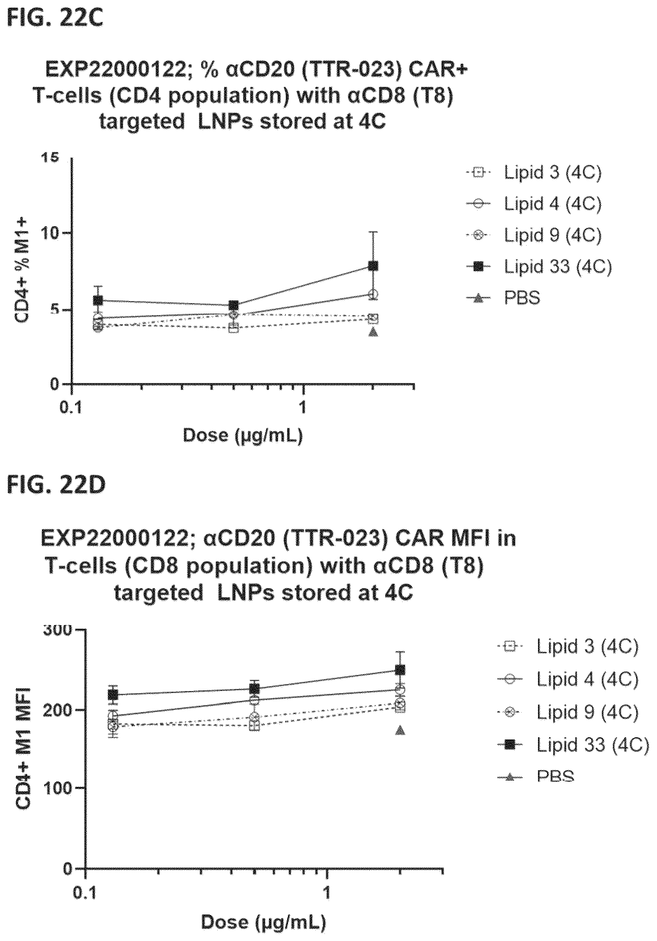

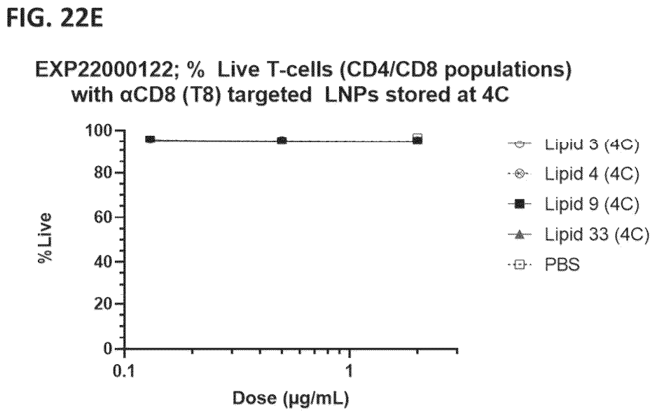

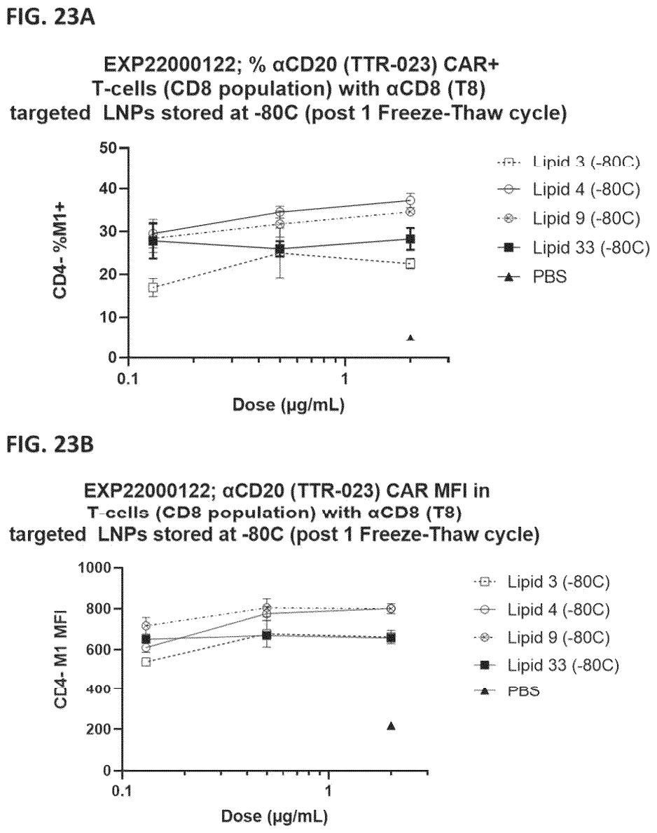

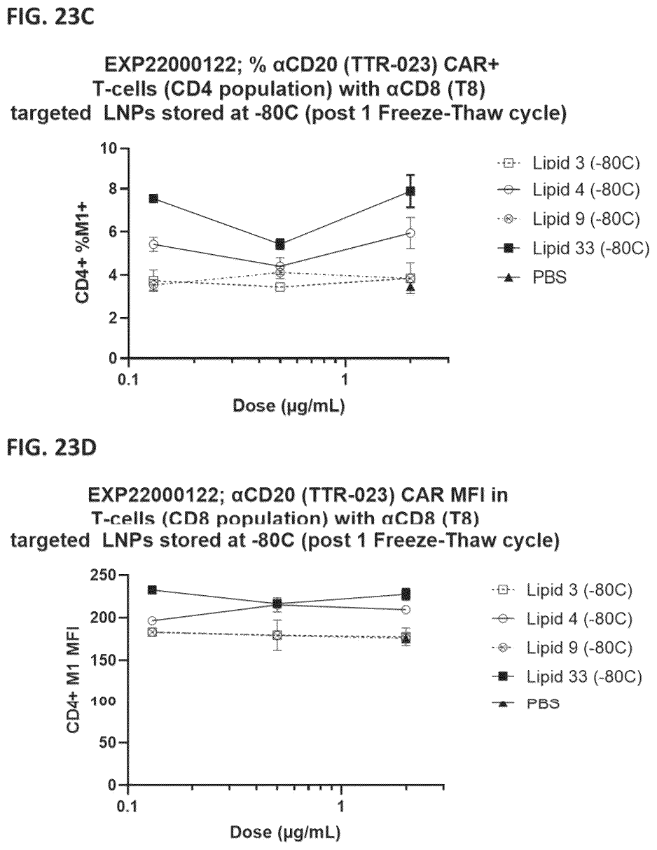

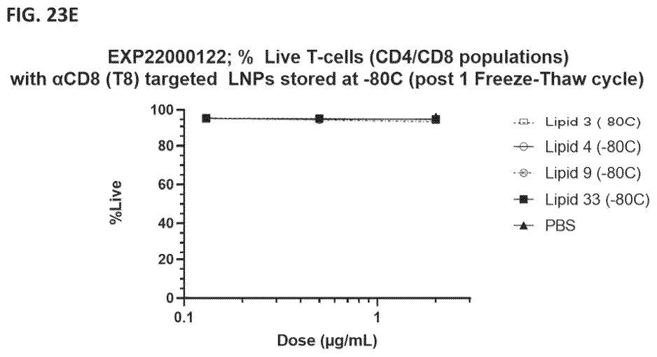

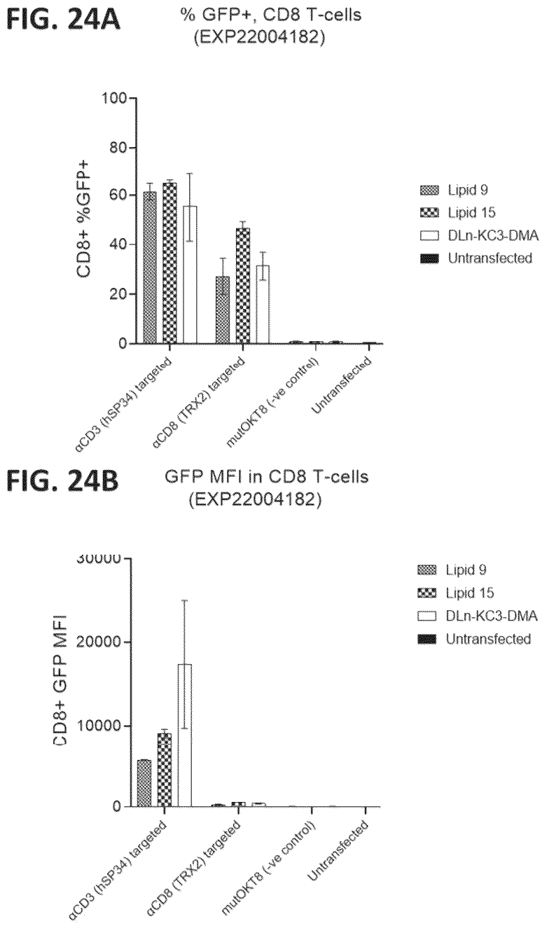

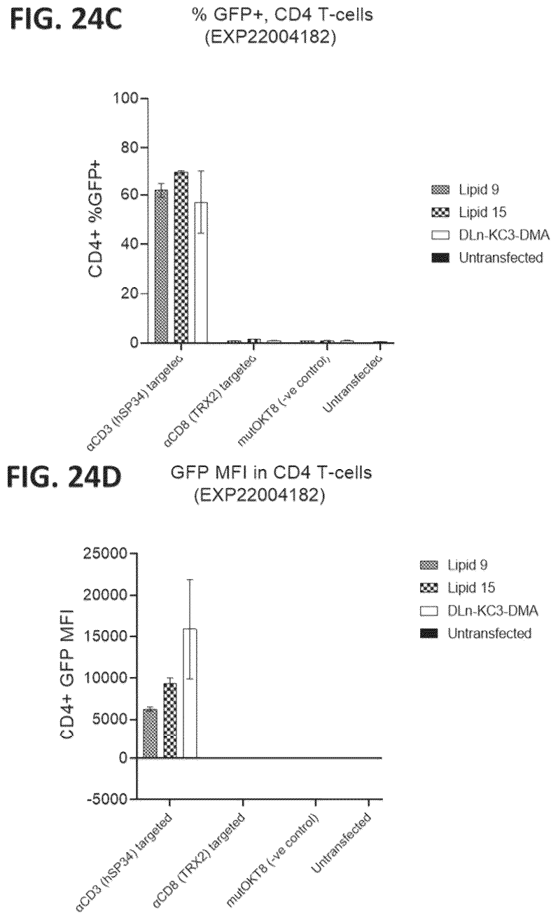

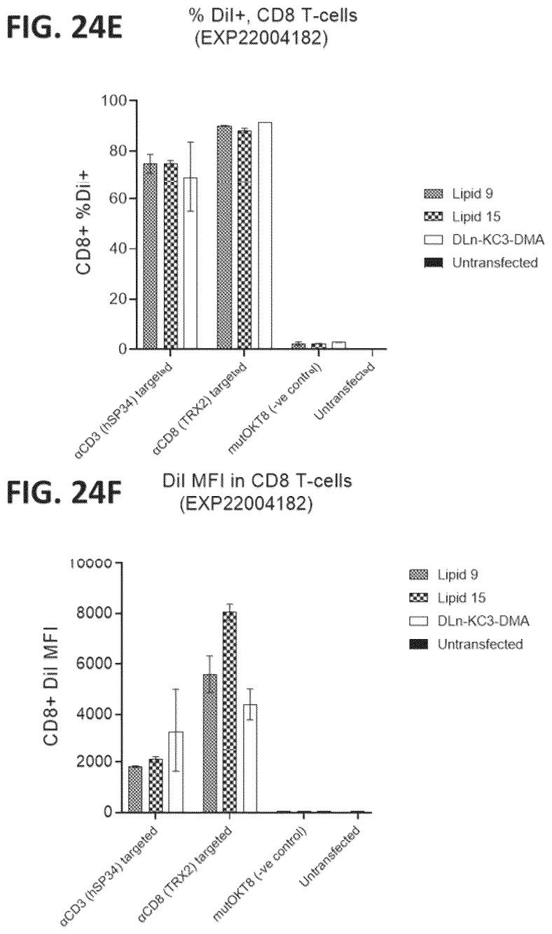

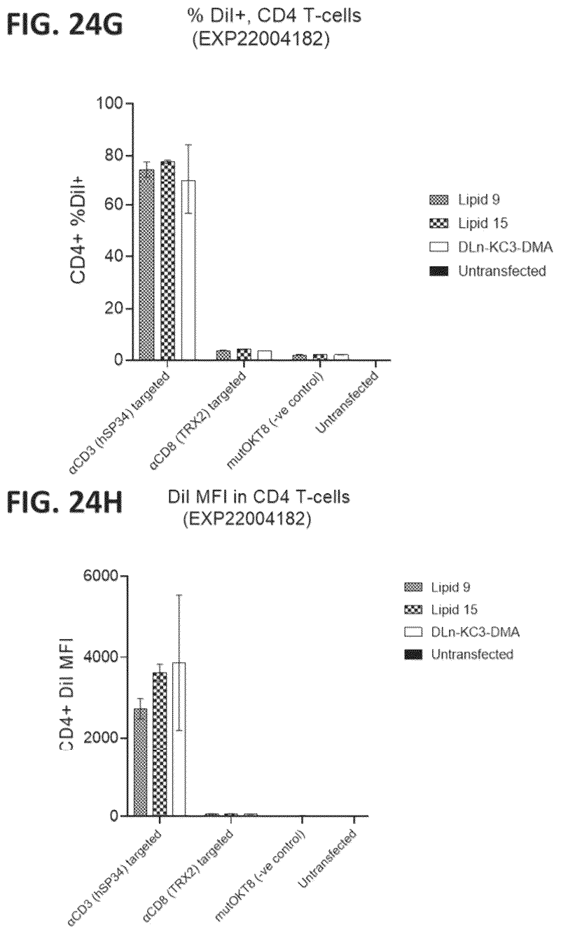

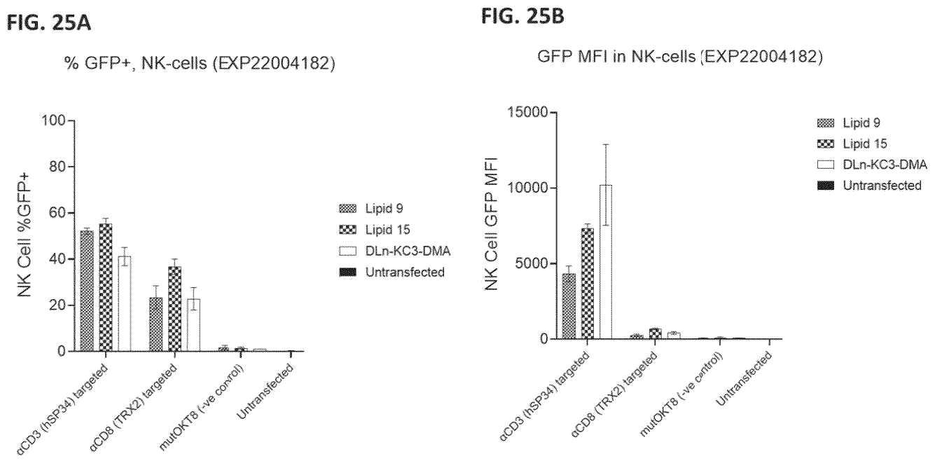

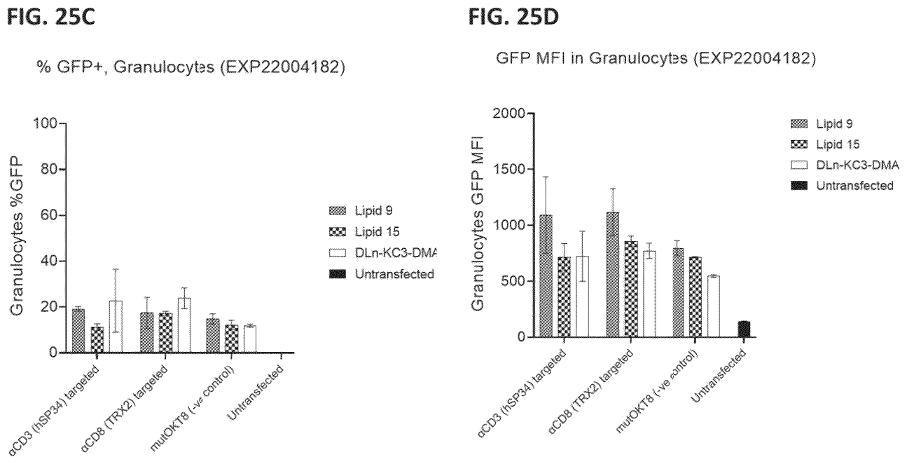

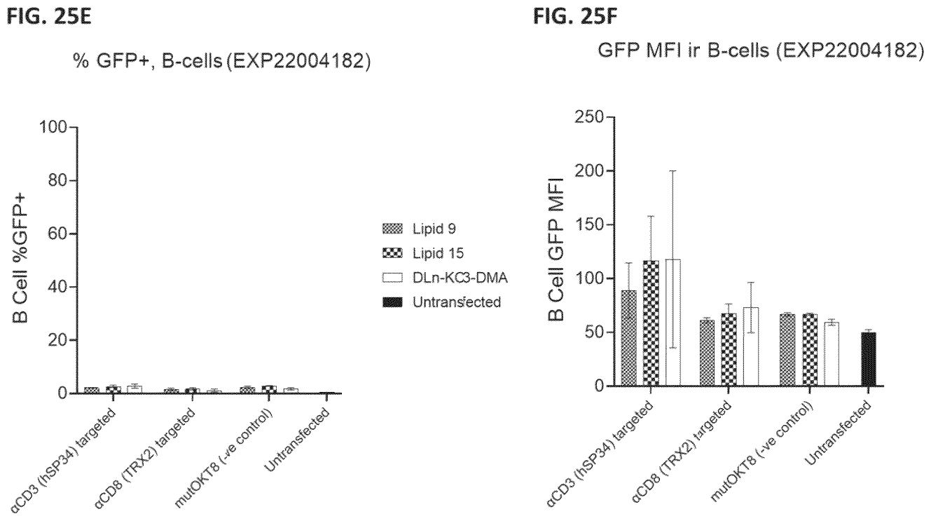

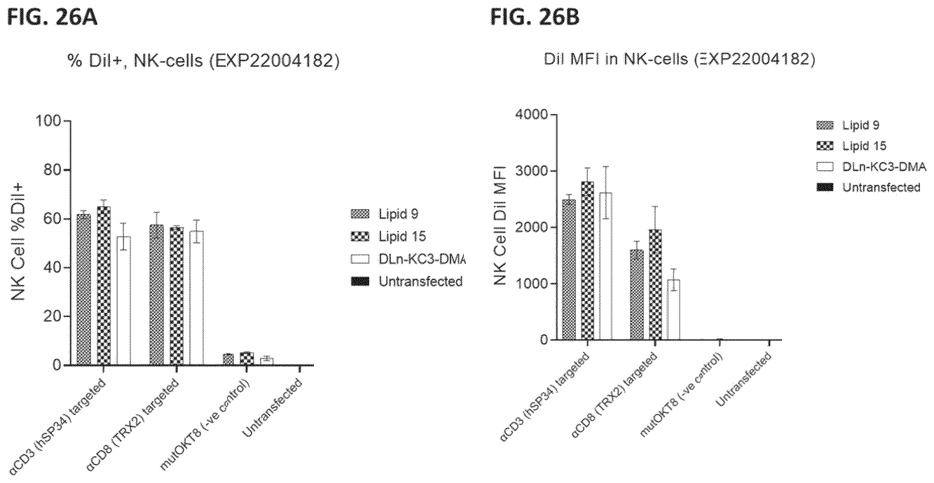

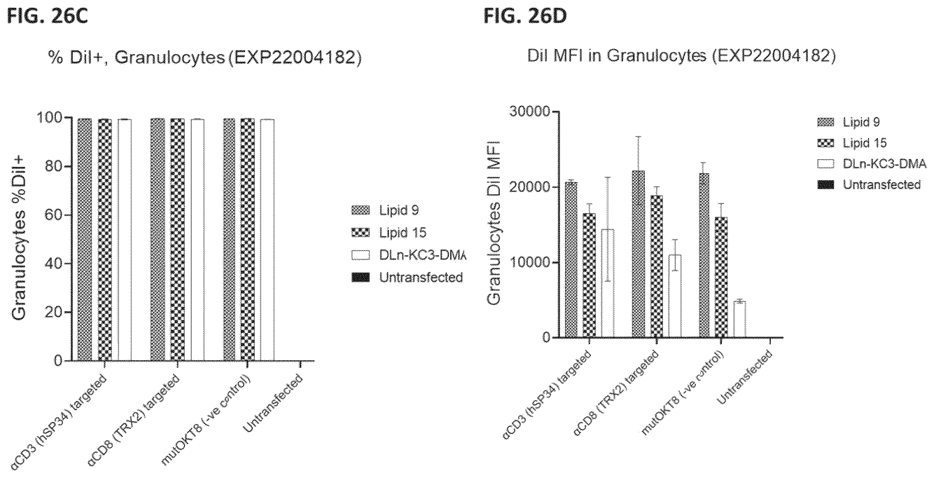

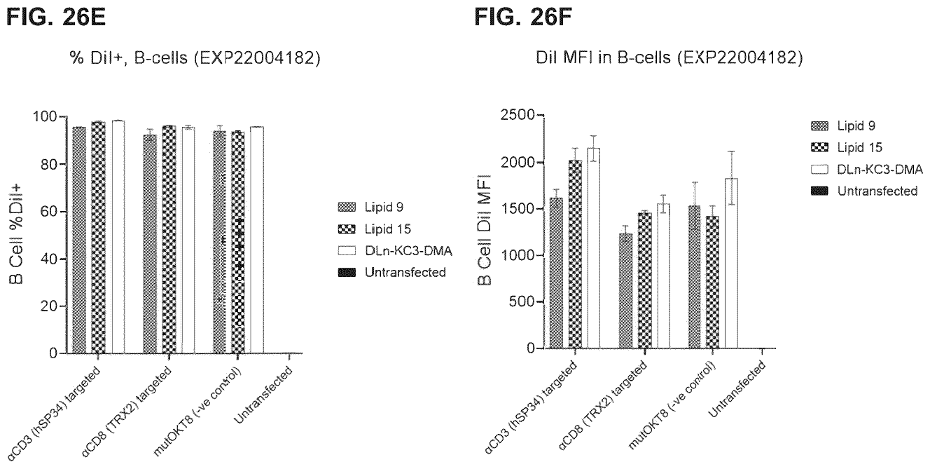

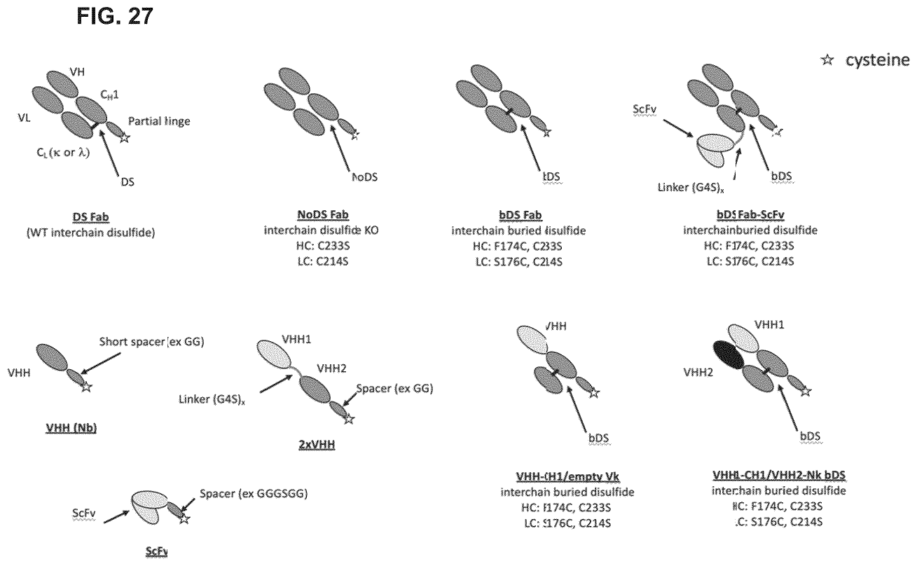

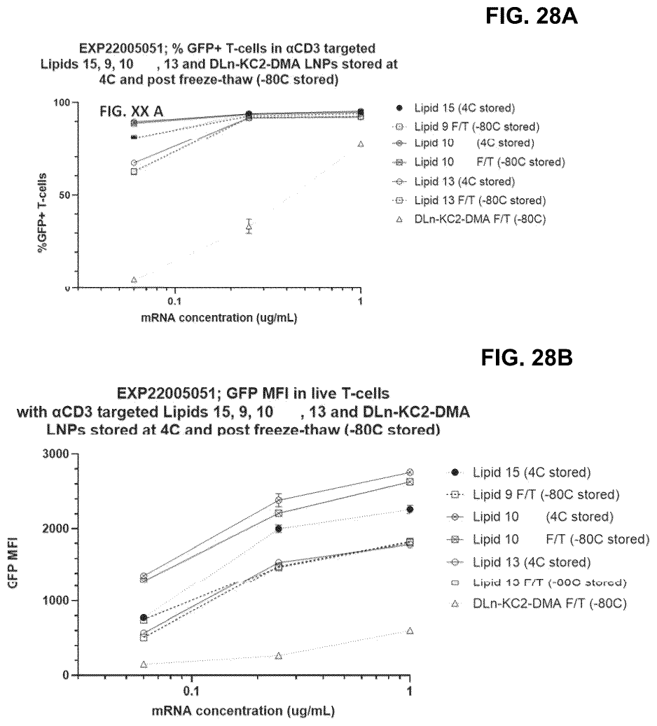

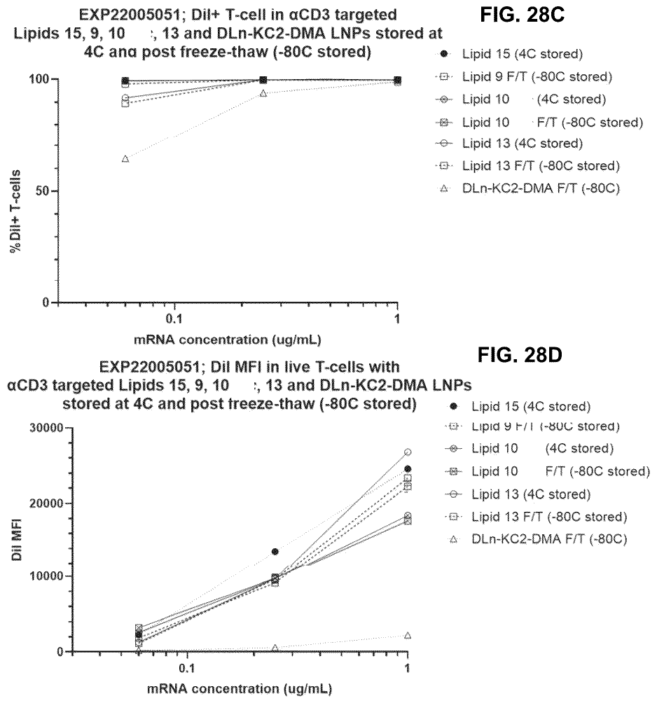

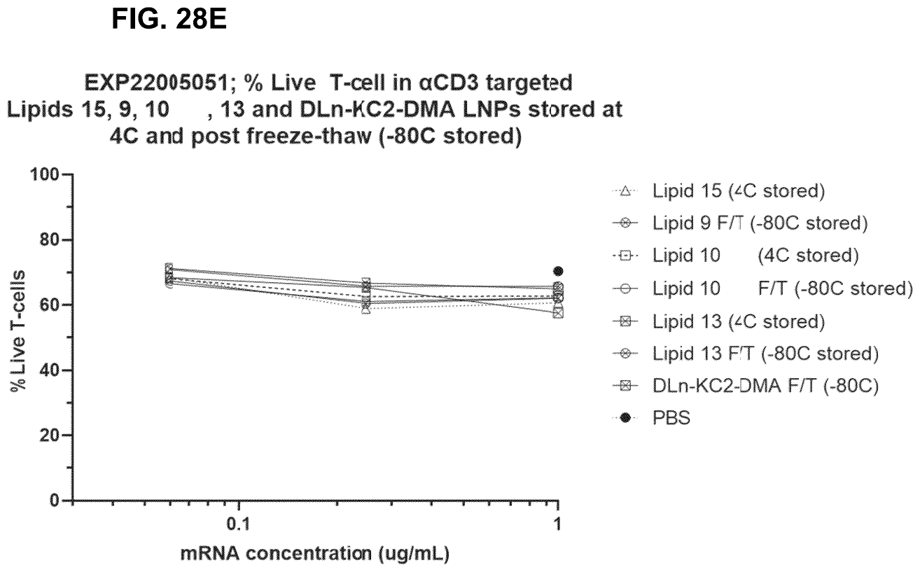

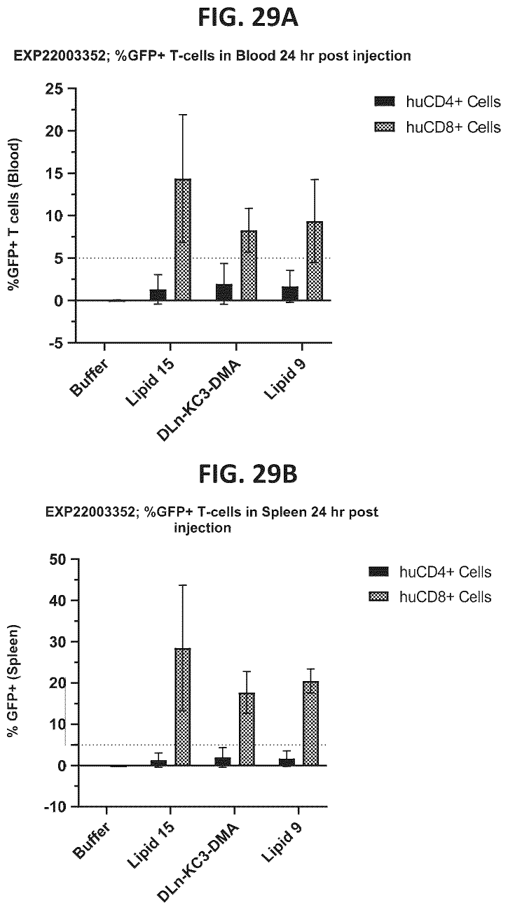

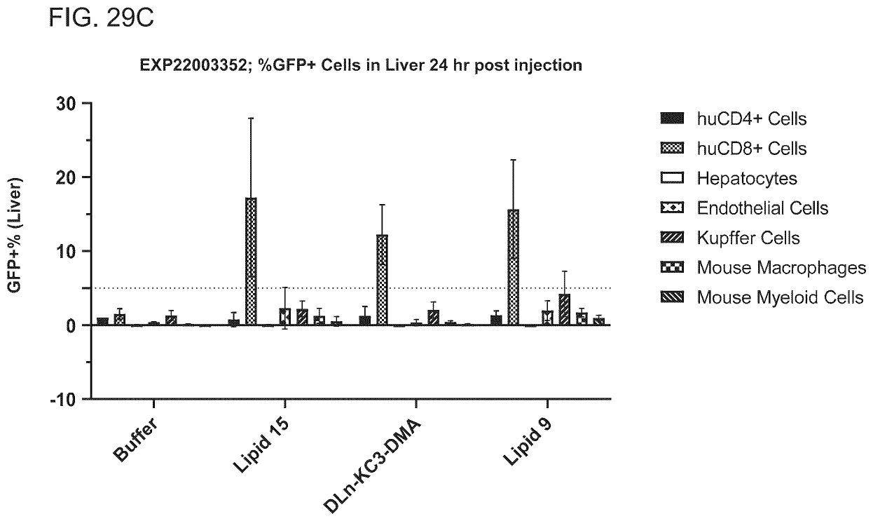

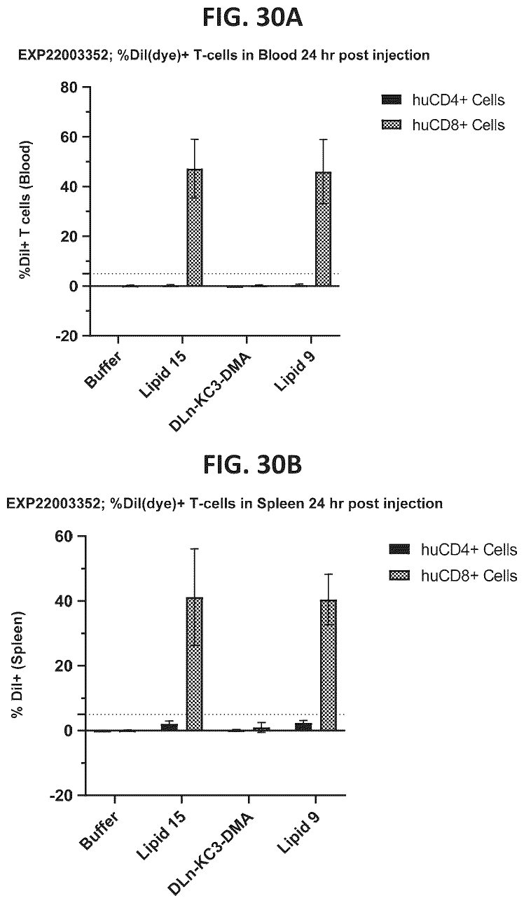

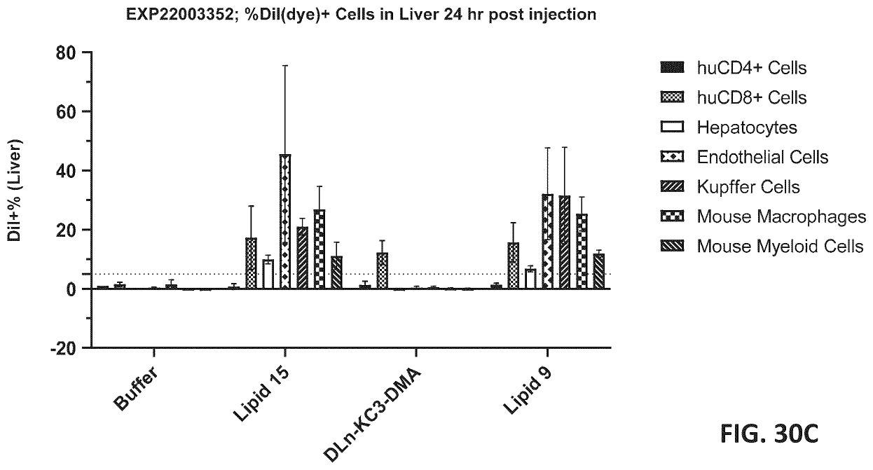

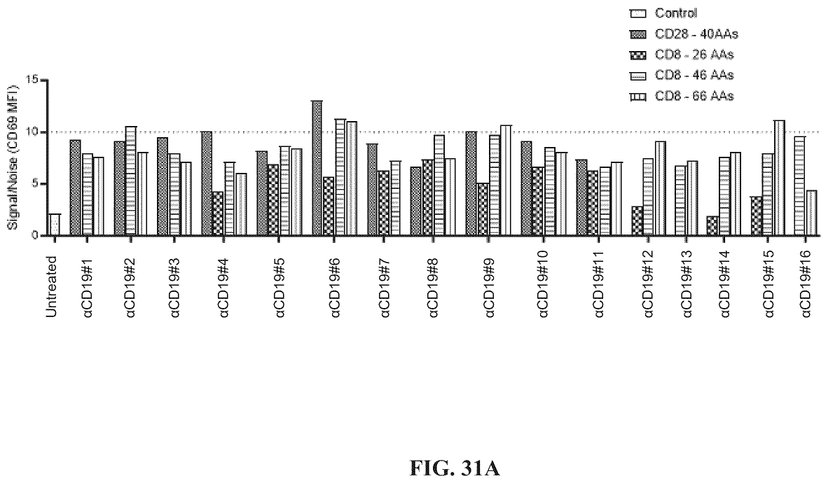

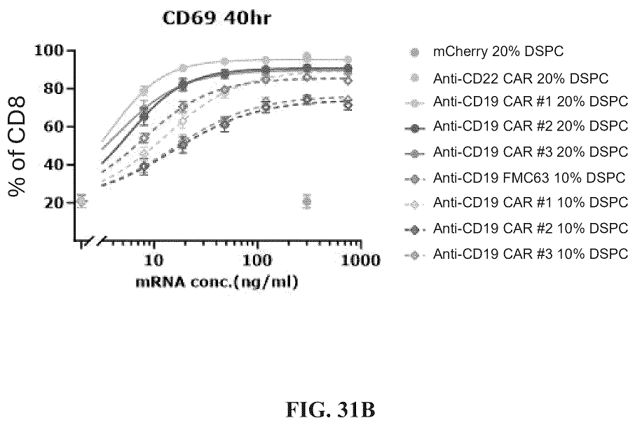

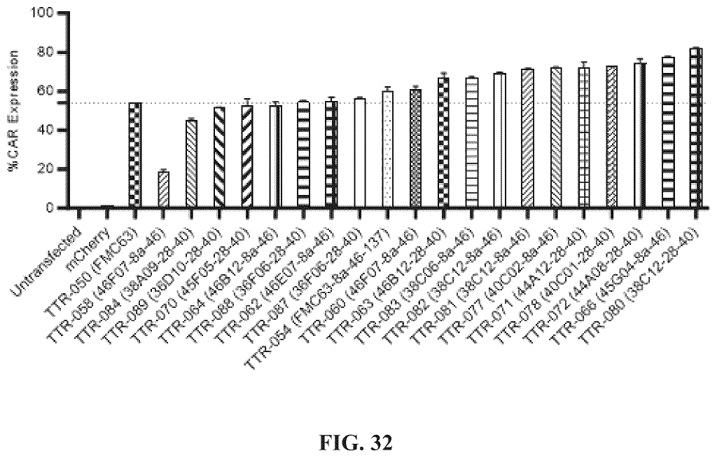

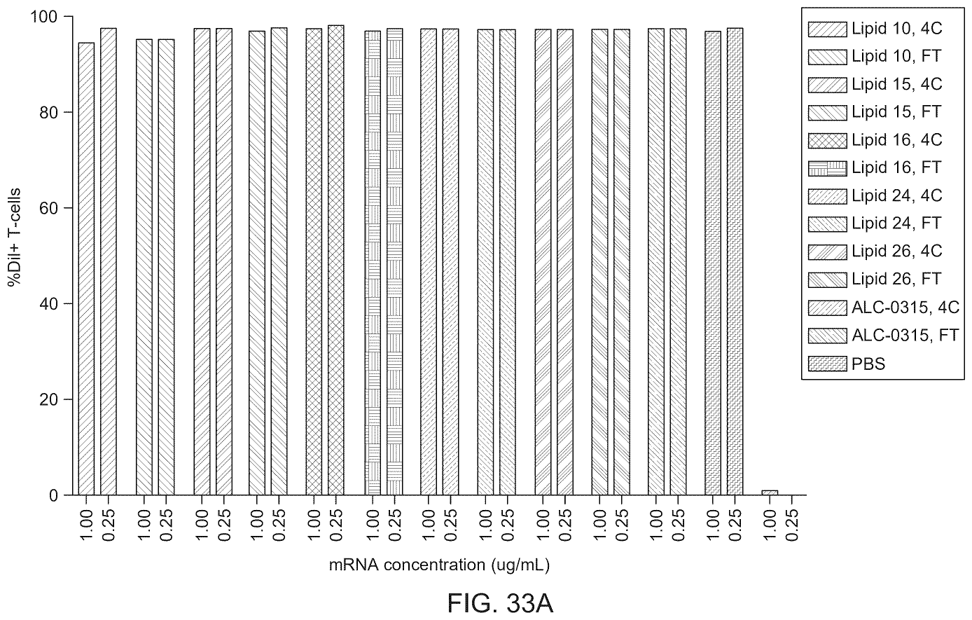

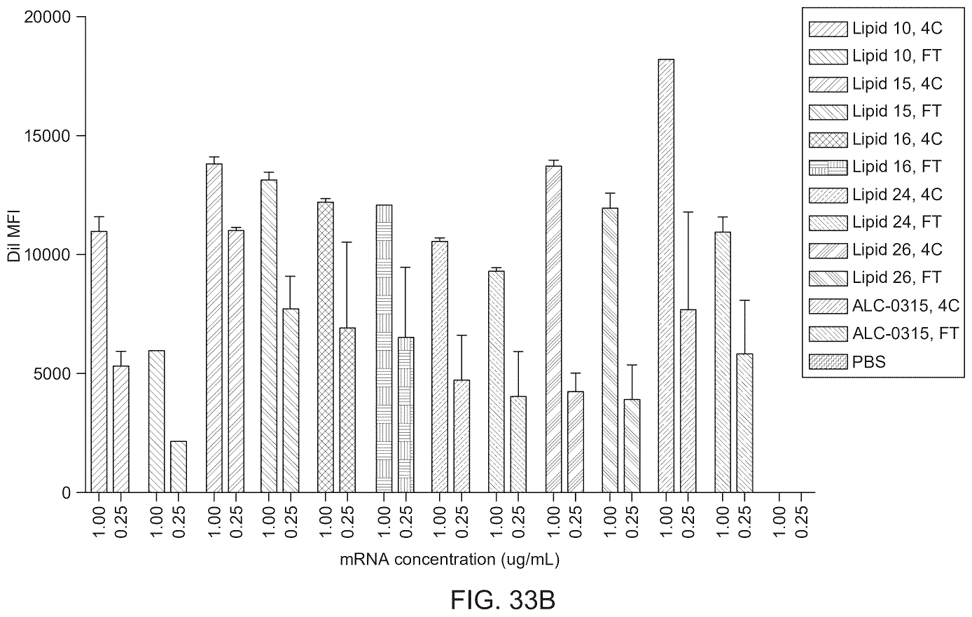

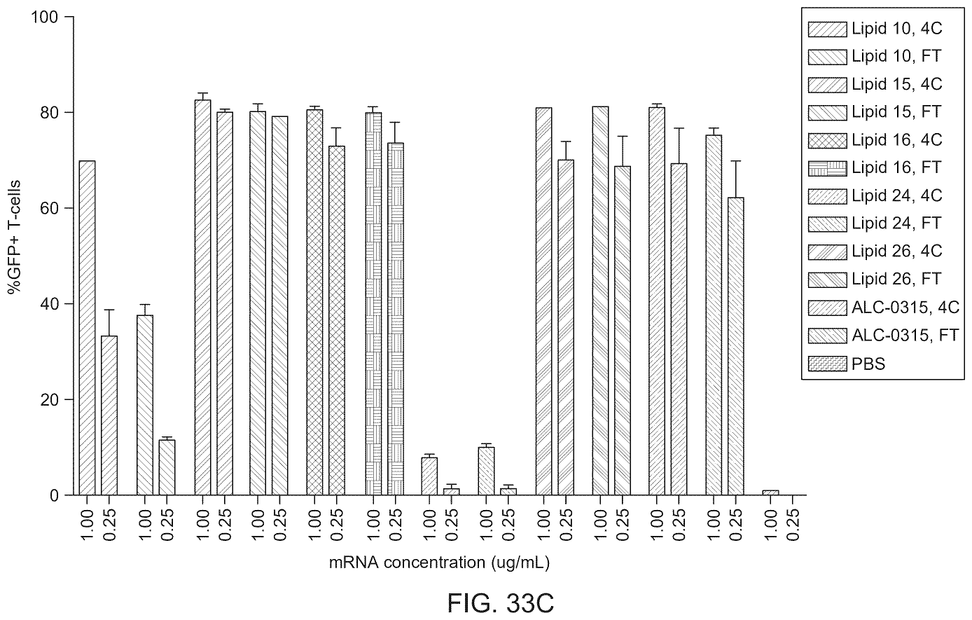

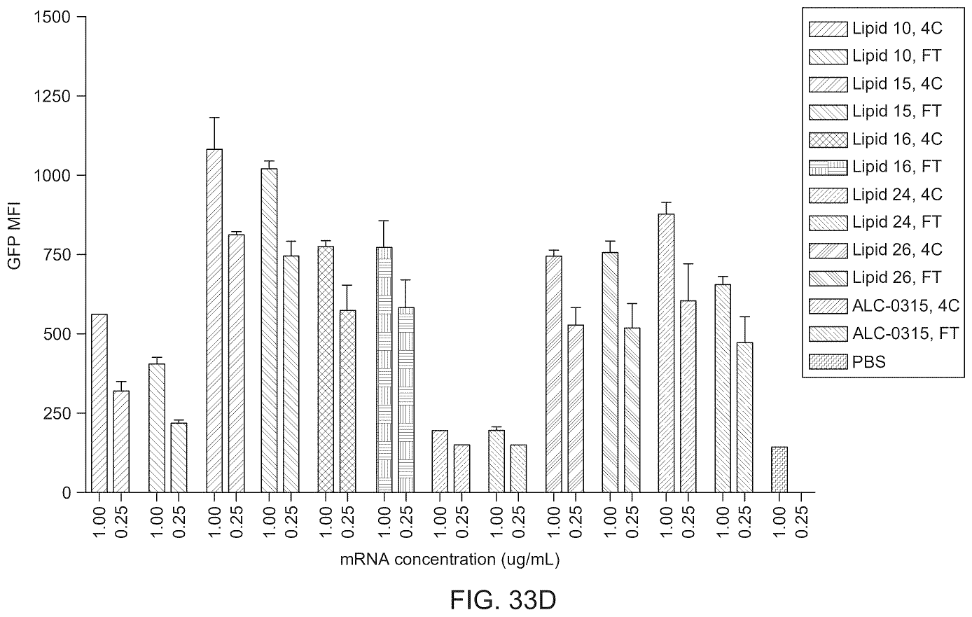

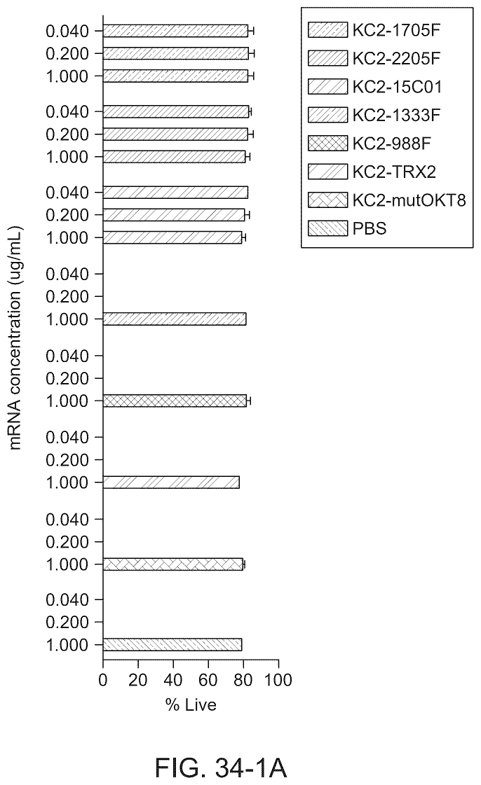

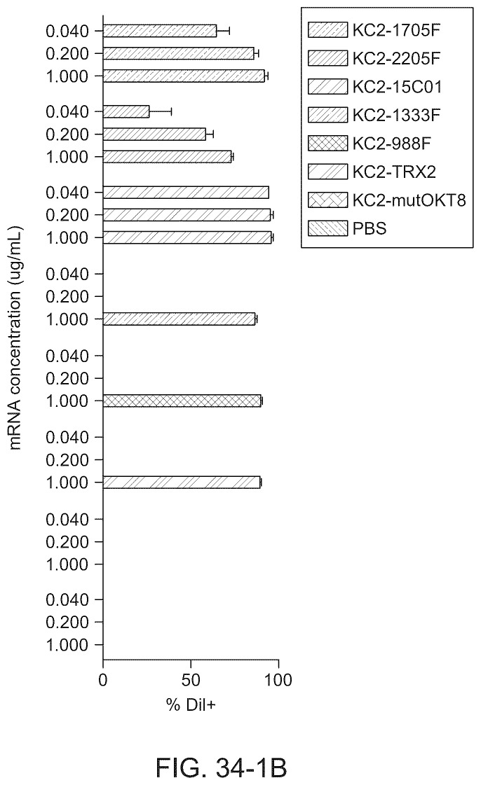

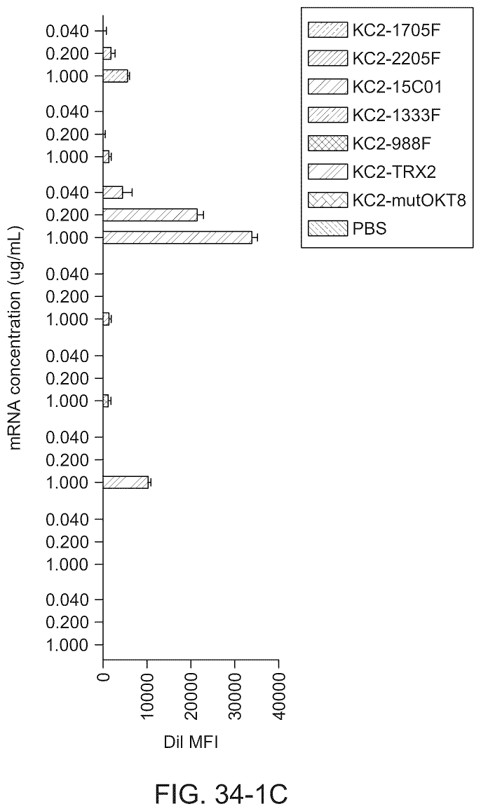

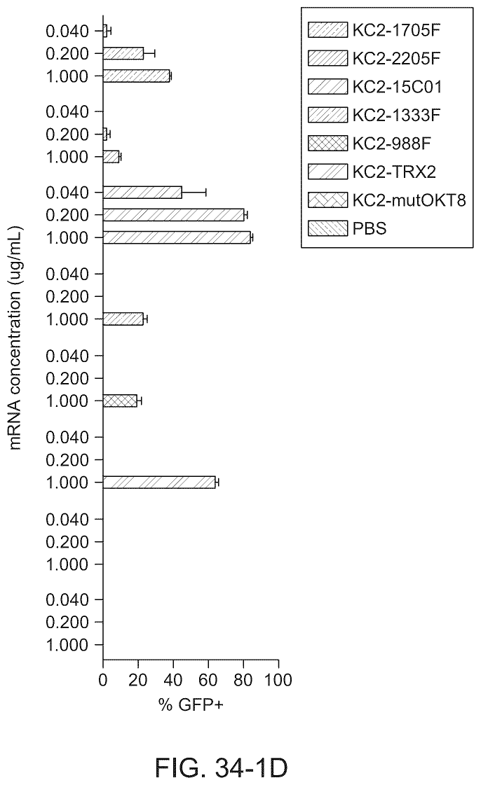

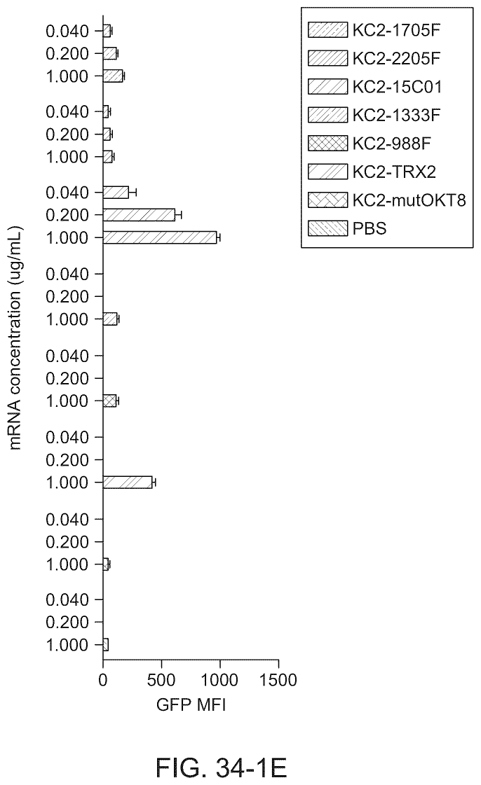

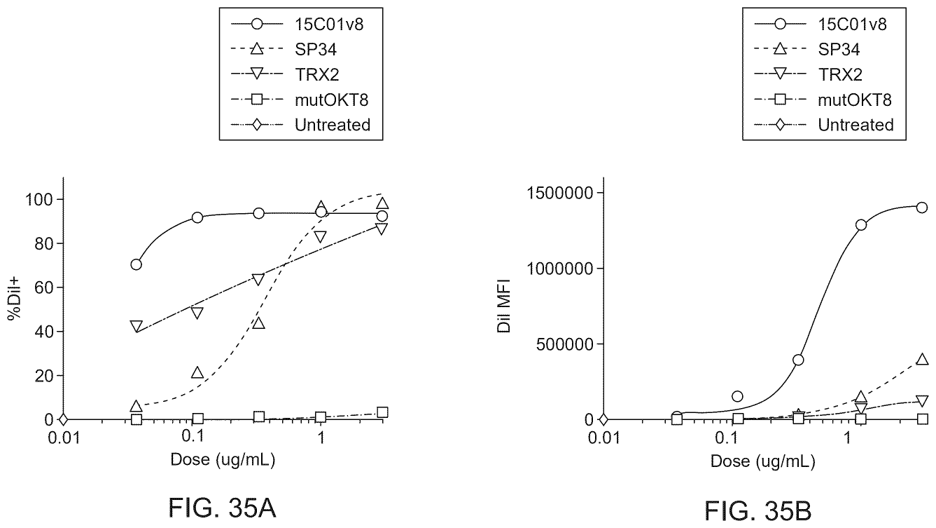

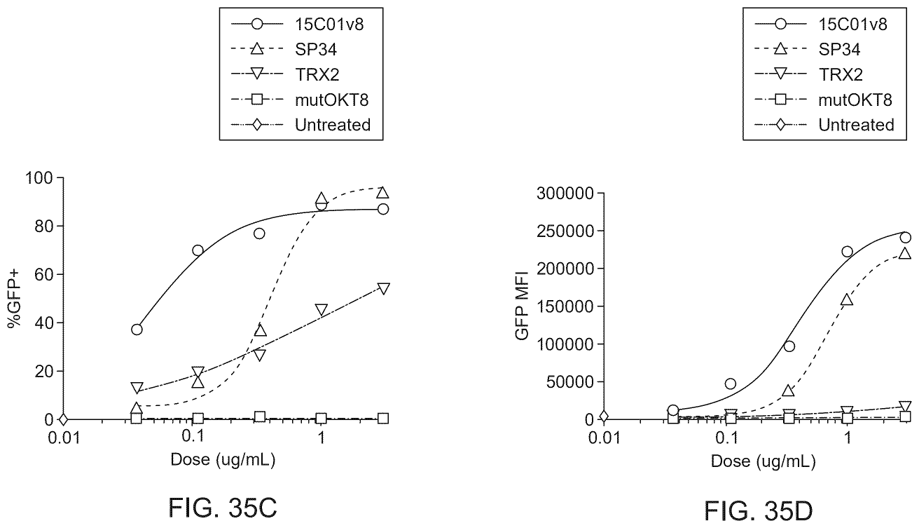

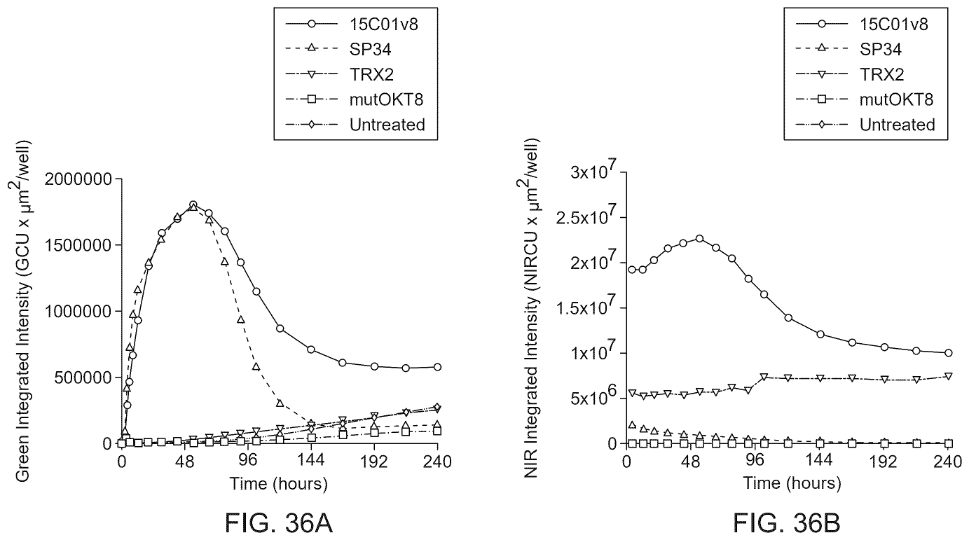

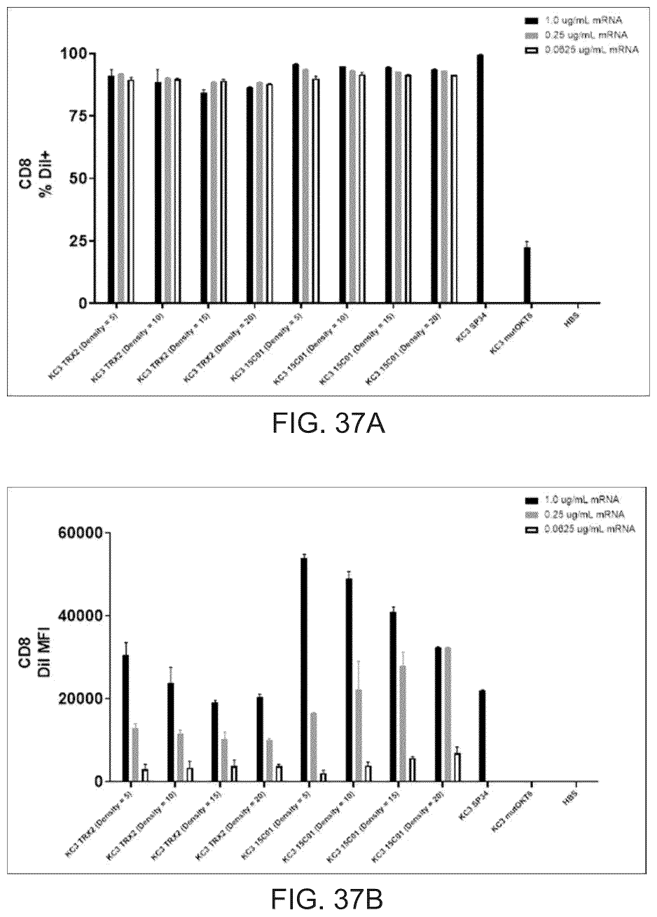

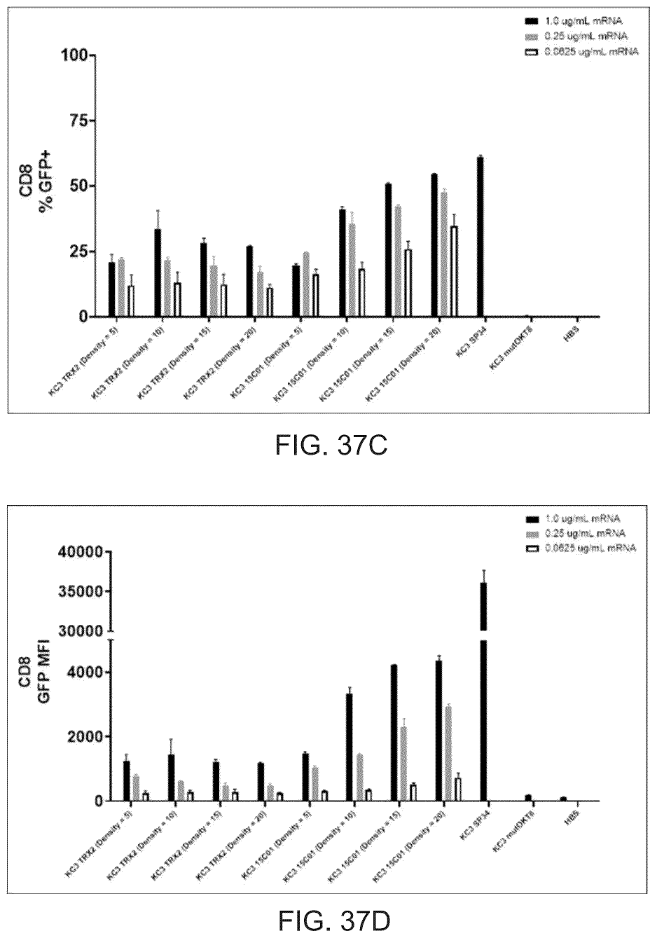

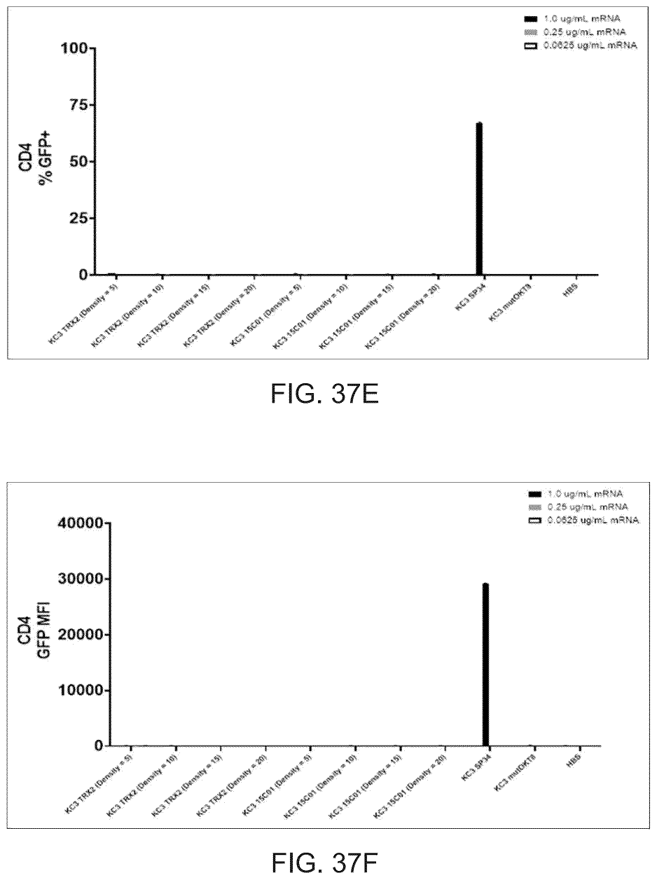

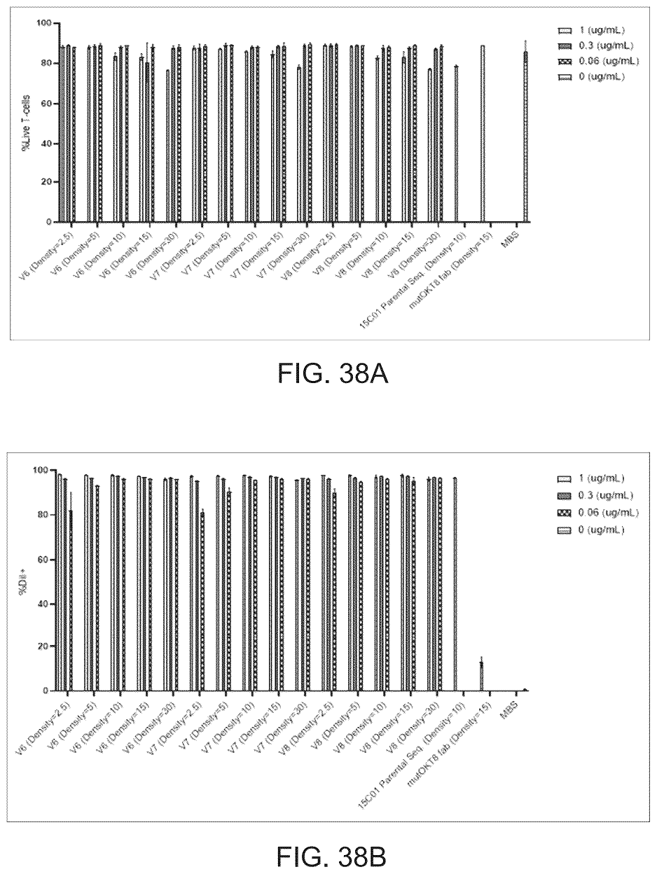

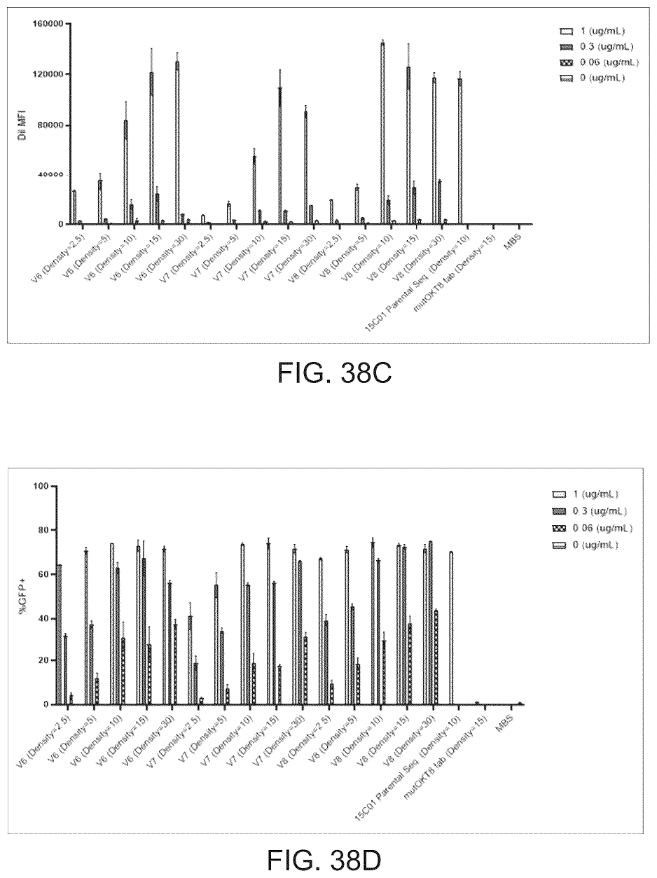

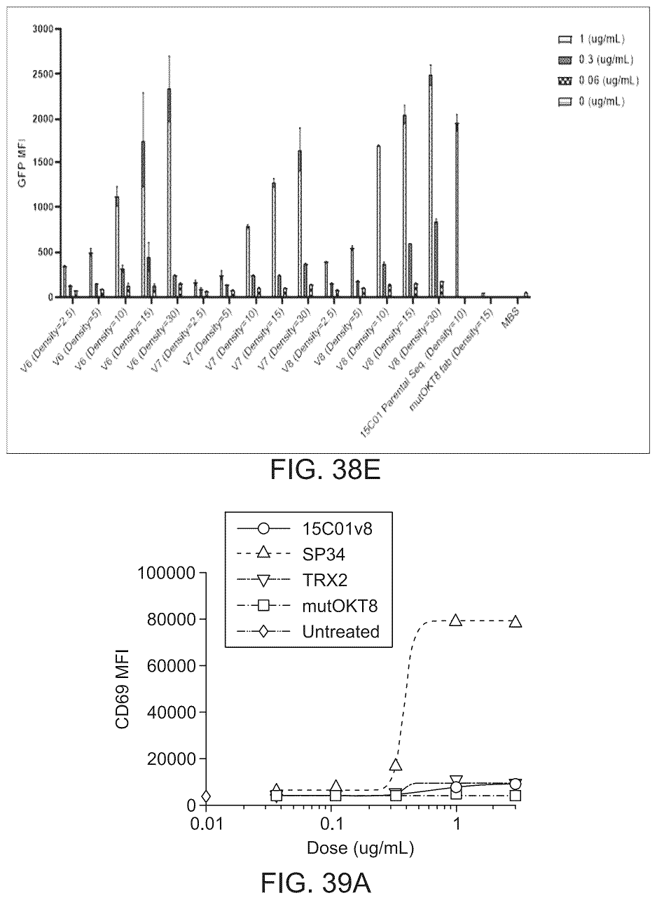

FIGS. 4P-1 and 4P-2 depict the proton NMR spectrum and LC-CAD chromatogram of Lipid 19. FIG. 4P-1 depicts proton NMR spectrum of Lipid 19; FIG. 4P-2 depicts the LC-ELSD chromatogram of Lipid 19.

FIGS. 4Q-1 and 4Q-2 depict the proton NMR spectrum and LC-CAD chromatogram of Lipid 20. FIG. 4Q-1 depicts proton NMR spectrum of Lipid 20; FIG. 4Q-2 depicts the LC-ELSD chromatogram of Lipid 20.

FIGS. 4R-1 and 4R-2 depict the proton NMR spectrum and LC-CAD chromatogram of Lipid 31. FIG. 4R-1 depicts proton NMR spectrum of Lipid 31; FIG. 4R-2 depicts the LC-CAD chromatogram of Lipid 31.

FIGS. 4S-1 and 4S-2 depict the proton NMR spectrum and LC-CAD chromatogram of Lipid 32. FIG. 4S-1 depicts proton NMR spectrum of Lipid 32; FIG. 4S-2 depicts the LC-CAD chromatogram of Lipid 32.

FIGS. 4T-1 and 4T-2 depict the proton NMR spectrum and LC-CAD chromatogram of Lipid 33. FIG. 4T-1 depicts proton NMR spectrum of Lipid 33; FIG. 4T-2 depicts the LC-CAD chromatogram of Lipid 33.

FIGS. 4U-1 and 4U-2 depict the proton NMR spectrum and LC-CAD chromatogram of Lipid 34. FIG. 4U-1 depicts proton NMR spectrum of Lipid 34; FIG. 4U-2 depicts the LC-CAD chromatogram of Lipid 34.

FIGS. 4V-1 and 4V-2 depict the proton NMR spectrum and LC-CAD chromatogram of Lipid 14A. FIG. 4V-1 depicts proton NMR spectrum of Lipid 14A; FIG. 4V-2 depicts the LC-CAD chromatogram of Lipid 14A.

FIGS. 4W-1 and 4W-2 depict the proton NMR spectrum and LC-CAD chromatogram of Lipid 17A. FIG. 4W-1 depicts proton NMR spectrum of Lipid 17A; FIG. 4W-2 depicts the LC-CAD chromatogram of Lipid 17A.

FIGS. 4X-1 and 4X-2 depict the proton NMR spectrum and LC-CAD chromatogram of Lipid 18A. FIG. 4X-1 depicts proton NMR spectrum of Lipid 18A; FIG. 4X-2 depicts the LC-CAD chromatogram of Lipid 18A.

FIGS. 4Y-1 and 4Y-2 depict the proton NMR spectrum and LC-CAD chromatogram of Lipid 21A. FIG. 4Y-1 depicts proton NMR spectrum of Lipid 21A; FIG. 4Y-2 depicts the LC-CAD chromatogram of Lipid 21A.

FIGS. 4Z-1 and 4Z-2 depict the proton NMR spectrum and LC-CAD chromatogram of Lipid 22. FIG. 4Z-1 depicts proton NMR spectrum of Lipid 22; FIG. 4Z-2 depicts the LC-CAD chromatogram of Lipid 22.

FIGS. 4AA-1 and 4AA-2 depict the proton NMR spectrum and LC-CAD chromatogram of Lipid 23A. FIG. 4AA-1 depicts proton NMR spectrum of Lipid 23A; FIG. 4AA-2 depicts the LC-CAD chromatogram of Lipid 23A.

FIGS. 4AC-1 and 4AC-2 depict the proton NMR spectrum and LC-CAD chromatogram of Lipid 25A. FIG. 4AC-1 depicts proton NMR spectrum of Lipid 25A; FIG. 4AC-2 depicts the LC-CAD chromatogram of Lipid 25A.

FIGS. 4AE-1 and 4AE-2 depict the proton NMR spectrum and LC-CAD chromatogram of Lipid 27. FIG. 4AE-1 depicts proton NMR spectrum of Lipid 27; FIG. 4AE-2 depicts the LC-CAD chromatogram of Lipid 27.

FIGS. 4AF-1 and 4AF-2 depict the proton NMR spectrum and LC-CAD chromatogram of Lipid 28. FIG. 4AF-1 depicts proton NMR spectrum of Lipid 28; FIG. 4AF-2 depicts the LC-CAD chromatogram of Lipid 28.

FIGS. 4AG-1 and 4AG-2 depict the proton NMR spectrum and LC-CAD chromatogram of Lipid 29. FIG. 4AG-1 depicts proton NMR spectrum of Lipid 29; FIG. 4AG-2 depicts the LC-CAD chromatogram of Lipid 29.

FIGS. 4AH-1 and 4AH-2 depict the proton NMR spectrum and LC-CAD chromatogram of Lipid 37A. FIG. 4AH-1 depicts proton NMR spectrum of Lipid 37A; FIG. 4AH-2 depicts the LC-CAD chromatogram of Lipid 37A.

FIGS. 4AI-1 and 4AI-2 depict the proton NMR spectrum and LC-CAD chromatogram of Lipid 19A. FIG. 4AI-1 depicts proton NMR spectrum of Lipid 19A; FIG. 4AI-2 depicts the LC-CAD chromatogram of Lipid 19A.

FIGS. 4AJ-1 and 4AJ-2 depict the proton NMR spectrum and LC-CAD chromatogram of Lipid 20A. FIG. 4AJ-1 depicts proton NMR spectrum of Lipid 20A; FIG. 4AJ-2 depicts the LC-CAD chromatogram of Lipid 20A.

FIG. 5A depicts diameter (DLS, nm) of LNPs based on Lipid 1 to Lipid 8 in pH 7.4 HBS, pH 6.5 MBS, Post antibody (αCD3, hSP34) insertion and post freeze-thaw (−80° C.).

FIG. 5B depicts polydispersity (DLS) of LNPs based on Lipid 1 to Lipid 8 in pH 7.4 HBS, pH 6.5 MBS, Post antibody (αCD3, hSP34) insertion and post freeze-thaw (−80° C.).

FIG. 5C depicts charge (Zeta potential, DLS) of LNPs based on Lipid 1 to Lipid 8 in pH 5.5 MBS, pH 7.4 HBS.

FIG. 5D depicts % RNA recovery and dye accessible RNA in LNPs based on Lipid 1 to Lipid 8.

FIG. 6A depicts diameter (DLS, nm) of LNPs based on Lipids 9, 10, 11, and 15 in pH 7.4 HBS, pH 6.5 MBS, Post antibody (αCD3, hSP34) insertion and post freeze-thaw (−80° C.).

FIG. 6B depicts polydispersity (DLS) of LNPs based on Lipids 9, 10, 11, and 15 in pH 7.4 HBS, pH 6.5 MBS, Post antibody (αCD3, hSP34) insertion and post freeze-thaw (−80° C.).

FIG. 6C depicts charge (Zeta potential, DLS) of LNPs based on Lipids 9, 10, 11, and 15 in pH 5.5 MBS, pH7.4 HBS.

FIG. 6D depicts % RNA recovery and dye accessible RNA in LNPs based on Lipids 9, 10, 11, and 15.

FIG. 7A depicts diameter (DLS, nm) of LNPs based on Lipid 31 to Lipid 34 in pH 7.4 HBS, pH 6.5 MBS, Post antibody (αCD3, hSP34) insertion and post freeze-thaw (−80° C.).

FIG. 7B depicts polydispersity (DLS) of LNPs based on Lipid 31 to Lipid 34 in pH 7.4 HBS, pH 6.5 MBS, Post antibody (αCD3, hSP34) insertion and post freeze-thaw (−80° C.).

FIG. 7C depicts charge (Zeta potential, DLS) of LNPs based on Lipid 31 to Lipid 34 in pH 5.5 MBS, pH 7.4 HBS.

FIG. 7D depicts % RNA recovery and dye accessible RNA in LNPs based on Lipid 31 to Lipid 34.

FIG. 8A depicts diameter (DLS, nm) of LNPs based on Lipids 1, 3, 4, 5, 9, and 15 in pH 7.4 HBS, pH 6.5 MBS, Post inserted with αCD8 antibody conjugates TRX-2 and T8.

FIG. 8B depicts polydispersity (DLS) of LNPs based on Lipids 1, 3, 4, 5, 9, and 15 in pH 7.4 HBS, pH 6.5 MBS, Post inserted with αCD8 antibody conjugates TRX-2 and T8.

FIG. 9A depicts diameter (DLS, nm) of LNPs based on Lipids 1, 8, 9, 10, 11, and 15 in pH 7.4 HBS, pH 6.5 MBS, Post inserted with αCD8 antibody conjugates TRX-2 and T8.

FIG. 9B depicts polydispersity (DLS) of LNPs based on Lipids 1, 8, 9, 10, 11, and 15 in pH 7.4 HBS, pH 6.5 MBS, Post inserted with αCD8 antibody conjugates TRX-2 and T8.

FIG. 10A depicts diameter (DLS, nm) of LNPs based on Lipid 3, 4, 33, and 34 in pH 7.4 HBS, pH 6.5 MBS, Post antibody (αCD3, hSP34) insertion and post freeze-thaw (−80° C.).

FIG. 10B depicts polydispersity (DLS) of LNPs based on Lipid 3, 4, 33, and 34 in pH 7.4 HBS, pH 6.5 MBS, Post antibody (αCD3, hSP34) insertion and post freeze-thaw (−80° C.).

FIG. 10C depicts charge (Zeta potential, DLS) of LNPs based on Lipid 3, 4, 33, and 34 in pH 5.5 MBS, pH7.4 HBS, pH6.5 MBS, post antibody (αCD3, hSP34) insertion and post freeze-thaw (−80° C.).

FIG. 10D depicts % RNA recovery and dye accessible RNA in LNPs based on Lipid 3, 4, 33, and 34.

FIG. 11A depicts GFP expression in primary human T cells; transfected by αCD8 (hsp34) targeted LNPs based on ALC-0315, DLin-MC3-DMA, Lipid 3, Lipid 6, and Lipid 7, stored at 4° C.; % GFP+ T cells at 24 hours.

FIG. 11B depicts GFP expression in primary human T cells; transfected by αCD3 (hsp34) targeted LNPs based on ALC-0315, DLin-MC3-DMA, Lipid 3, Lipid 6, and Lipid 7, after 1 freeze-thaw cycle (−80° C. storage); % GFP+ T cells at 24 hours.

FIG. 11C depicts GFP expression in primary human T cells; transfected by αCD3 (hsp34) targeted LNPs based on ALC-0315, DLin-MC3-DMA, Lipid 3, Lipid 6, and Lipid 7, stored at 4° C.; GFP MFI in live T cells at 24 hours.

FIG. 11D depicts GFP expression in primary human T cells; transfected by αCD3 (hsp34) targeted LNPs based on ALC-0315, DLin-MC3-DMA, Lipid 3, Lipid 6, and Lipid 7, after 1 freeze-thaw cycle (−80° C. storage); GFP MFI in live T cells at 24 hours.

FIG. 11E depicts % live T cells transfected by αCD3 (hsp34) targeted LNPs based on ALC-0315, DLin-MC3-DMA, Lipid 3, Lipid 6, and Lipid 7, after 1 freeze-thaw cycle (−80° C. storage); % live T cells at 24 hours.

FIG. 12A depicts GFP expression in primary human T cells; transfected by αCD3 (hsp34) targeted LNPs based on SM-102, DLin-KC2-DMA, Lipid 3, Lipid 4 stored at 4° C.; % GFP+ T cells at 24 hours.

FIG. 12B depicts GFP expression in primary human T cells; transfected by αCD3 (hsp34) targeted LNPs based on SM-102, DLin-KC2-DMA, Lipid 3, Lipid 4, after 1 freeze-thaw cycle (−80° C. storage); % GFP+ T cells at 24 hours.

FIG. 12C depicts GFP expression in primary human T cells; transfected by αCD3 (hsp34) targeted LNPs based on SM-102, DLin-KC2-DMA, Lipid 3, Lipid 4, stored at 4° C.; GFP MFI in live T cells at 24 hours.

FIG. 12D depicts GFP expression in primary human T cells; transfected by αCD3 (hsp34) targeted LNPs based on SM-102, DLin-KC2-DMA, Lipid 3, Lipid 4, after 1 freeze-thaw cycle (−80° C. storage); GFP MFI in live T cells at 24 hours.

FIG. 12E depicts % live T cells transfected with by αCD3 (hsp34) targeted LNPs based on SM-102, DLin-KC2-DMA, Lipid 3, Lipid 4, after 1 freeze-thaw cycle (−80° C. storage); % live T cells at 24 hours.

FIG. 13A depicts GFP expression in primary human T cells; transfected by targeted LNPs based on DLin-KC2-DMA (−80° C. stored), Lipid 1 (4° C. stored), Lipid 3 (4° C. stored), and Lipid 5 (4° C. stored); % GFP+ T cells.

FIG. 13B depicts GFP expression in primary human T cells; transfected by targeted LNPs based on DLin-KC2-DMA, Lipid 1, Lipid 3, and Lipid 5 after freeze-thaw cycle (−80° C. storage); % GFP+ T cells.

FIG. 13C depicts GFP expression in primary human T cells; transfected by targeted LNPs based on DLin-KC2-DMA (−80° C. stored), Lipid 1 (4° C. stored), Lipid 3 (4° C. stored), and Lipid 5 (4° C. stored), GFP MFI in live T cells.

FIG. 13D depicts GFP expression in primary human T cells; transfected by a targeted LNPs based on DLin-KC2-DMA, Lipid 1, Lipid 3, and Lipid 5 after freeze-thaw cycle (−80° C. storage); GFP MFI in live T cells.

FIG. 13E depicts % live T cells transfected with targeted LNPs based on DLin-KC2-DMA, Lipid 1, Lipid 3, and Lipid 5 stored at −80° C.

FIG. 14A depicts GFP expression in primary human T cells; transfected by αCD3 (hSP34) targeted LNPs based on DLin-KC2-DMA, Lipid 1 (4° C. stored), Lipid 8 (4° C. stored), and Lipid 8 (−80° C. stored); % GFP+ T cells.

FIG. 14B depicts GFP expression in primary human T cells; transfected by αCD3 (hSP34) targeted LNPs based on DLin-KC2-DMA, Lipid 1 (4° C. stored), Lipid 8 (4° C. stored), and Lipid 8 (−80° C. stored); GFP MFI in live T cells.

FIG. 14C depicts % living cells with targeted LNPs based on DLin-KC2-DMA, Lipid 1 (4° C. stored), Lipid 8 (4° C. stored), and Lipid 8 (−80° C. stored).

FIG. 15A depicts GFP expression in primary human T cells; transfected by αCD3 (hsp34) targeted LNPs based on DLin-KC2-DMA (−80° C. stored), Lipid 8 (4° C. stored), Lipid 9 (4° C. stored), and Lipid 10 (4° C. stored); % GFP+ T cells.

FIG. 15B depicts GFP expression in primary human T cells; transfected by αCD3 (hsp34) targeted LNPs based on DLin-KC2-DMA (−80° C. stored), Lipid 8 (−80° C. stored) and Lipid 10 (−80° C. stored); % GFP+ T cells.

FIG. 15C depicts GFP expression in primary human T cells; transfected by αCD3 (hsp34) targeted LNPs based on DLin-KC2-DMA (−80° C. stored), Lipid 8 (4° C. stored), Lipid 9 (4° C. stored), and Lipid 10 (4° C. stored); GFP MFI in live T cells.

FIG. 15D depicts GFP expression in primary human T cells; transfected by αCD3 (hsp34) targeted LNPs based on DLin-KC2-DMA (−80° C. stored), Lipid 8 (−80° C. stored) and Lipid 10 (−80° C. stored); GFP MFI in live T cells.

FIG. 15E depicts % live T cells transfected with αCD3 (hsp34) targeted LNPs based on DLin-KC2-DMA (−80° C. stored), Lipid 8 (4° C. stored), Lipid 9 (4° C. stored), and Lipid 10 (4° C. stored); % live T cells.

FIG. 15F depicts % live T cells transfected with αCD3 (hsp34) targeted LNPs based on DLin-KC2-DMA (−80° C. stored), Lipid 8 (−80° C. stored) and Lipid 10 (−80° C. stored); % live T cells.

FIG. 16A depicts GFP expression in primary human T cells; transfected by αCD3 (hsp34) targeted LNPs based on DLin-KC2-DMA (−80° C. stored), Lipid 3 (4° C. stored), Lipid 4 (4° C. stored), Lipid 9 (4° C. stored), Lipid 15 (4° C. stored); % GFP+ T cells.

FIG. 16B depicts GFP expression in primary human T cells; transfected by αCD3 (hsp34) targeted LNPs based on DLin-KC2-DMA (−80° C. stored), Lipid 3 (−80° C. stored), Lipid 4 (−80° C. stored), Lipid 9 (−80° C. stored), Lipid 15 (−80° C. stored); % GFP+ T cells.

FIG. 16C depicts GFP expression in primary human T cells; transfected by αCD3 (hsp34) targeted LNPs based on DLin-KC2-DMA (−80° C. stored), Lipid 3 (4° C. stored), Lipid 4 (4° C. stored), Lipid 9 (4° C. stored), Lipid 15 (4° C. stored), GFP MFI in live T cells.

FIG. 16D depicts GFP expression in primary human T cells; transfected by αCD3 (hsp34) targeted LNPs based on DLin-KC2-DMA (−80° C. stored), Lipid 3 (−80° C. stored), Lipid 4 (−80° C. stored), Lipid 9 (−80° C. stored), Lipid 15 (−80° C. stored); GFP MFI in live T cells.

FIG. 16E depicts % live T cells transfected with αCD3 (hsp34) targeted LNPs based on DLin-KC2-DMA (−80° C. stored), Lipid 3 (−80° C. stored), Lipid 4 (−80° C. stored), Lipid 9 (−80° C. stored), and Lipid 15 (−80° C. stored).

FIG. 17A depicts GFP expression in primary human T cells; transfected by αCD8 (TRX2) targeted LNPs based on Lipid 3, Lipid 4, Lipid 9, Lipid 15, and compared with the corresponding non-targeted parent LNPs; % GFP+ T cells.

FIG. 17B depicts GFP expression in primary human T cells; transfected by αCD8 (TRX2) targeted LNPs based on Lipid 3, Lipid 4, Lipid 9, Lipid 15, and compared with the corresponding non-targeted parent LNPs; GFP MFI in live T cells.

FIG. 17C depicts %+Dil T cell with αCD8 (TRX2) targeted LNPs based on Lipid 3, Lipid 4, Lipid 9, Lipid 15, and compared with the corresponding non-targeted parent LNPs.

FIG. 17D depicts Dil MFI in live T cells with αCD8 (TRX2) targeted LNPs based on Lipid 3, Lipid 4, Lipid 9, Lipid 15, and compared with the corresponding non-targeted parent LNPs.

FIG. 17E depicts % live T cells transfected with αCD8 (TRX2) targeted LNPs based on Lipid 3, Lipid 4, Lipid 9, Lipid 15, and compared with the corresponding non-targeted parent LNPs.

FIG. 18A depicts GFP expression in primary human T cells; transfected by αCD8 (T8) targeted LNPs based on Lipid 3, Lipid 4, Lipid 9, Lipid 15, and compared with the corresponding non-targeted parent LNPs; % GFP+ T cells.

FIG. 18B depicts GFP expression in primary human T cells; transfected by αCD8 (T8) targeted LNPs based on Lipid 3, Lipid 4, Lipid 9, Lipid 15, and compared with the corresponding non-targeted parent LNPs; GFP MFI in live T cells.

FIG. 18C depicts %+Dil T cell with αCD8 (T8) targeted LNPs based on Lipid 3, Lipid 4, Lipid 9, Lipid 15, and compared with the corresponding non-targeted parent LNPs.

FIG. 18D depicts Dil MFI in live T cells with αCD8 (T8) targeted LNPs based on Lipid 3, Lipid 4, Lipid 9, Lipid 15, and compared with the corresponding non-targeted parent LNPs.

FIG. 18E depicts % live T cells transfected with αCD8 (T8) targeted LNPs based on Lipid 3, Lipid 4, Lipid 9, Lipid 15, and compared with the corresponding non-targeted parent LNPs.

FIG. 19A depicts GFP expression in primary human T cells; transfected by αCD3 (hSP34) targeted LNPs based on DLin-KC2-DMA, Lipid 2, Lipid 3, Lipid 31, and Lipid 32; stored at 4° C.; % GFP+ T cells.

FIG. 19B depicts GFP expression in primary human T cells; transfected by αCD3 (hSP34) targeted LNPs based on DLin-KC2-DMA, Lipid 2, Lipid 3, Lipid 31, and Lipid 32; stored at 4° C.; GFP MFI in live T cells.

FIG. 19C depicts % living T cells with αCD3 (hSP34) targeted LNPs based on DLin-KC2-DMA, Lipid 2, Lipid 3, Lipid 31, and Lipid 32; stored at 4° C.

FIG. 20A depicts GFP expression in primary human T cells; transfected with αCD3 (hSP34) targeted LNPs based on DLin-KC2-DMA (−80° C. stored), Lipid 3 (4° C. stored), Lipid 33 (4° C. stored), Lipid 34 (4° C. stored), or transfected with αCD8 (muOKT8) targeted LNPs based on Lipid 33 (4° C. stored) Lipid 34 (4° C. stored); % GFP+ T cells.

FIG. 20B depicts GFP expression in primary human T cells; transfected with αCD3 (hSP34) targeted LNPs based on DLin-KC2-DMA (−80° C. stored), Lipid 3 (−80° C. stored), Lipid 33 (−80° C. stored), Lipid 34 (−80° C. stored); % GFP+ T cells.

FIG. 20C depicts GFP expression in primary human T cells; transfected with αCD3 (hSP34) targeted LNPs based on DLin-KC2-DMA (−80° C. stored), Lipid 3 (−80° C. stored), Lipid 33 (4° C. stored), Lipid 34 (4° C. stored), or transfected with αCD8 (muOKT8) targeted LNPs based on Lipid 33 (4° C. stored) Lipid 34 (4° C. stored); GFP MFI in live T cells.

FIG. 20D depicts GFP expression in primary human T cells; transfected with αCD3 (hSP34) targeted LNPs based on DLin-KC2-DMA (−80° C. stored), Lipid 3 (−80° C. stored), Lipid 33 (−80° C. stored), Lipid 34 (−80° C. stored); GFP MFI in live T cells.

FIG. 20E depicts % live T cells transfected with αCD3 (hSP34) targeted LNPs based on DLin-KC2-DMA (−80° C. stored), Lipid 3 (−80° C. stored), Lipid 33 (−80° C. stored), Lipid 34 (−80° C. stored), or transfected with αCD8 (muOKT8) targeted LNPs based on Lipid 33 (4° C. stored) and Lipid 34 (4° C. stored).

FIG. 21A depicts % αCD20 CAR+ T cells transfected by αCD3 (hSP34) targeted LNPs based on Lipid 3 (4° C. stored), Lipid 4 (4° C. stored), Lipid 9 (4° C. stored), and Lipid 33 (4° C. stored); as illustrated by % M1 value.

FIG. 21B depicts % αCD20 CAR+ T cells transfected by αCD3 (hSP34) targeted LNPs based on Lipid 3 (−80° C. stored), Lipid 4 (−80° C. stored), Lipid 9 (−80° C. stored), Lipid 33 (−80° C. stored); as illustrated by % M1 value.

FIG. 21C depicts % αCD20 CAR MFI in T cells transfected by αCD3 (hSP34) targeted LNPs based on Lipid 3 (4° C. stored), Lipid 4 (4° C. stored), Lipid 9 (4° C. stored), and Lipid 33 (4° C. stored).

FIG. 21D depicts % αCD20 CAR MFI in T cells transfected by αCD3 (hSP34) targeted LNPs based on Lipid 3 (−80° C. stored), Lipid 4 (−80° C. stored), Lipid 9 (−80° C. stored), Lipid 33 (−80° C. stored).

FIG. 21E depicts % live T cells transfected with αCD3 (hSP34) targeted αCD20 CAR LNPs based on Lipid 3 (4° C. stored), Lipid 4 (4° C. stored), Lipid 9 (4° C. stored), and Lipid 33 (4° C. stored).

FIG. 21F depicts % live T cells transfected with αCD3 (hSP34) targeted αCD20 CAR LNPs based on Lipid 3 (−80° C. stored), Lipid 4 (−80° C. stored), Lipid 9 (−80° C. stored), Lipid 33 (−80° C. stored).

FIG. 22A depicts % αCD20 CAR+ T cells (CD8 population) with αCD8 (T8) targeted LNPs based on Lipid 3 (4° C. stored), Lipid 4 (4° C. stored), Lipid 9 (4° C. stored), Lipid 33 (4° C. stored), as illustrated by CD4-% M1 value.

FIG. 22B depicts αCD20 CAR MFI in T cells (CD8 population) with αCD8 (T8) targeted LNPs based on Lipid 3 (4° C. stored), Lipid 4 (4° C. stored), Lipid 9 (4° C. stored), Lipid 33 (4° C. stored), as illustrated by CD4-M1 MFI value.

FIG. 22C depicts αCD20 CAR level in CD4+ T cells transfected with αCD8 (T8) targeted LNPs based on Lipid 3 (4° C. stored), Lipid 4 (4° C. stored), Lipid 9 (4° C. stored), Lipid 33 (4° C. stored); as illustrated by the M1% value.

FIG. 22D depicts αCD20 CAR level in CD4+ T cells transfected with αCD8 (T8) targeted LNPs based on DLin-KC2-DMA (−80° C. stored), Lipid 3 (−80° C. stored), Lipid 33 (−80° C. stored), Lipid 34 (−80° C. stored); as illustrated by the M1 MFI value.

FIG. 22E depicts % live T cells (CD4/CD8 populations) transfected with αCD8 (T8) targeted αCD20 CAR LNPs based on Lipid 3 (4° C. stored), Lipid 4 (4° C. stored), Lipid 9 (4° C. stored), Lipid 33 (4° C. stored).

FIG. 23A depicts % αCD20 CAR+ T cells (CD8 population) transfected with αCD8 (T8) targeted LNPs based on Lipid 3 (−80° C. stored), Lipid 4 (−80° C. stored), Lipid 9 (−80° C. stored), Lipid 33 (−80° C. stored) after one Freeze-Thaw cycle, as illustrated by CD4-% M1 value.

FIG. 23B depicts αCD20 CAR MFI in T cells (CD8 population) transfected with αCD8 (T8) targeted LNPs based on Lipid 3 (−80° C. stored), Lipid 4 (−80° C. stored), Lipid 9 (−80° C. stored), Lipid 33 (−80° C. stored) after one Freeze-Thaw cycle, as illustrated by CD4-M1 MFI value.

FIG. 23C depicts αCD20 CAR level in CD4+ T cells transfected with αCD8 (T8) targeted LNPs based on Lipid 3 (−80° C. stored), Lipid 4 (−80° C. stored), Lipid 9 (−80° C. stored), Lipid 33 (−80° C. stored); as illustrated by CD4+% M1 value.

FIG. 23D depicts αCD20 CAR level in CD4+ T cells transfected with αCD8 (T8) targeted LNPs based on Lipid 3 (−80° C. stored), Lipid 4 (−80° C. stored), Lipid 9 (−80° C. stored), Lipid 33 (−80° C. stored); as illustrated by CD4+M1 MFI value.

FIG. 23E depicts % live T cells transfected with αCD8 (T8) targeted αCD20 CAR LNPs based on Lipid 3 (−80° C. stored), Lipid 4 (−80° C. stored), Lipid 9 (−80° C. stored), Lipid 33 (−80° C. stored).

FIG. 24A depicts GFP expression in CD8+ T cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % GFP+ T cells.

FIG. 24B depicts GFP expression in CD8+ T cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by GFP MFI.

FIG. 24C depicts GFP expression in CD4+ T cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % GFP+ T cells.

FIG. 24D depicts GFP expression in CD4+ T cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by GFP MFI.

FIG. 24E depicts % Dil+CD8+ T cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % Dil+ T cells.

FIG. 24F depicts Dil MFI in CD8+ T cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by Dil MFI.

FIG. 24G depicts % Dil+CD4+ T cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % Dil+ T cells.

FIG. 24H depicts Dil MFI in CD4+ T cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by Dil MFI.

FIG. 25A depicts GFP expression in NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % GFP+NK cells.

FIG. 25B depicts GFP expression in NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by GFP MFI.

FIG. 25C depicts GFP expression in granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % GFP+ granulocytes.

FIG. 25D depicts GFP expression in granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by GFP MFI.

FIG. 25E depicts GFP expression in B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % GFP+ B cells.

FIG. 25F depicts GFP expression in B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by GFP MFI.

FIG. 26A depicts LNP binding to NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % Dil+NK cells.

FIG. 26B depicts LNP binding to NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by Dil MFI.

FIG. 26C depicts LNP binding to granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % Dil+ granulocytes.

FIG. 26D depicts LNP binding to granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by Dil MFI.

FIG. 26E depicts LNP binding to B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % Dil+ B cells.

FIG. 26F depicts LNP binding to B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by Dil MFI.

FIG. 27 depicts structures of various Fab, VHH (Nb), ScFv, Fab-ScFv and Fab-VHH hybrids.

FIG. 28A depicts GFP expression in T cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by % GFP+ T cells.

FIG. 28B depicts GFP expression in T cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by GFP MFI.

FIG. 28C depicts % Dil+ T cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by % Dil+ T cells.

FIG. 28D depicts Dil MFI in live T cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by Dil % MFI.

FIG. 28E depicts % live T cells transfected with αCD3 targeted LNPs based on Lipid 9, Lipid 10, Lipid 13, Lipid 15, or DLin-KC2-DMA, stored at either 4° C. or post freeze-thaw (−80° C. stored).

FIGS. 29A-29C depict % GFP+ T cells (CD4 and CD8 populations) in Blood (FIG. 29A), Spleen (FIG. 29B), and Liver (FIG. 29C) samples (analyzed for additional cell types of interest per legend) at 24 hours post injection of GFP RNA using Lipid 15, DLin-KC3-DMA, and Lipid 9 LNP formulations and α-CD8 targeting with TRX-2 antibody.

FIGS. 30A-30C depict % DiI+ T cells (CD4 and CD8 populations) in Blood (FIG. 30A), Spleen (FIG. 30B), and Liver (FIG. 30C) samples (analyzed for additional cell types of interest per legend) at 24 hours post injection of GFP RNA using Lipid 15 (DiI-dye labelled), DLin-KC3-DMA (No DiI-dye label used), and Lipid 9 LNP (Dil-dye labelled) formulations and α-CD8 targeting with TRX-2 antibody.

FIGS. 31A-31B provide a comparison of the CD69 signal in Jurkat T cells (FIG. 31A) and primary T cells (FIG. 31B) resulting from anti-CD19 CAR mRNA transfection and co-culture with CD19-expressing Raji cells. Untreated Jurkat T cells or primary T cells were used as a negative control (i.e., signal/noise ratio of 1). Anti-CD19 CARs were designed using any one of αCD19 #1-16 VHH clones and a CD28 hinge of 40 amino acids (“AAs”), a CD8α hinge of 26 amino acids (i.e., “short”), a CD8a hinge of 26 amino acids (i.e., “intermediate”), or CD8a hinge of 66 amino acids (i.e., “long”).

FIG. 32 shows the anti-CD19 CAR protein expression levels after 24 hours in primary human CD8+ T cells resulting from CAR mRNA transfection. FMC63 was used as a benchmark control.

FIG. 33A depicts % DiI+ CD8+ T cells transfected with αCD8 targeted LNPs based on Lipids 10, 15, 16, 24, and 26 and ALC-0315 as a comparator, as illustrated by % DiI+ T cells.

FIG. 33B depicts DiI MFI of CD8+ T cells transfected with αCD8 targeted LNPs based on Lipids 10, 15, 16, 24, and 26 and ALC-0315 as a comparator, as illustrated by Dil MFI.

FIG. 33C depicts % GFP+ CD8+ T cells transfected with αCD8 targeted LNPs based on Lipids 10, 15, 16, 24, and 26 and ALC-0315 as a comparator, as illustrated by % GFP+ T cells.

FIG. 33D depicts GFP MFI of CD8+ T cells transfected with αCD8 targeted LNPs based on Lipids 10, 15, 16, 24, and 26 and ALC-0315 as a comparator, as illustrated by GFP MFI.

FIG. 34-1A depicts the viability of CD8+ T cells transfected with αCD8 targeted LNPs based on DLn-KC2-DMA, as illustrated by % Live T cells.

FIG. 34-1B depicts % DiI+ CD8+ T cells transfected with αCD8 targeted LNPs based on DLn-KC2-DMA, as illustrated by % DiI+ T cells.

FIG. 34-1C depicts DiI MFI in CD8+ T cells transfected with αCD8 targeted LNPs based on DLn-KC2-DMA, as illustrated by DiI MFI.

FIG. 34-1D depicts GFP expression in CD8+ T cells transfected with αCD8 targeted LNPs based on DLn-KC2-DMA, as illustrated by % GFP+ T cells.

FIG. 34-1E depicts GFP MFI in CD8+ T cells transfected with αCD8 targeted LNPs based on DLn-KC2-DMA, as illustrated by GFP MFI.

FIG. 35A depicts Dil expression in CD8+ T cells transfected with αCD3- and αCD8-targeted LNPs based on Lipid 15, at mRNA doses of 3, 1, 0.33, 0.11, and 0.037 μg/mL, as illustrated by % DiI+ T cells.

FIG. 35B depicts DiI MFI in CD8+ T cells transfected with αCD3- and αCD8-targeted LNPs based on Lipid 15, at mRNA doses of 3, 1, 0.33, 0.11, and 0.037 μg/mL, as illustrated by DiI MFI.

FIG. 35C depicts GFP expression in CD8+ T cells transfected with αCD3- and αCD8-targeted LNPs based on Lipid 15, at mRNA doses of 3, 1, 0.33, 0.11, and 0.037 μg/mL, as illustrated by % GFP+ T cells.

FIG. 35D depicts GFP MFI in CD8+ T cells transfected with αCD3- and αCD8-targeted LNPs based on Lipid 15, at mRNA doses of 3, 1, 0.33, 0.11, and 0.037 μg/mL, as illustrated by GFP MFI.

FIG. 36A depicts GFP expression in CD8+ T cells transfected with αCD3- and αCD8-targeted LNPs based on Lipid 15, at various time points, as illustrated by Green Integrated Intensity.

FIG. 36B depicts level of LNP association (Dil signal) in CD8+ T cells transfected with αCD3- and αCD8-targeted LNPs based on Lipid 15, at various time points, as illustrated by NIR Integrated Intensity.

FIG. 37A depicts level of LNP association (Dil signal) in CD8+ T cells transfected with DLin-KC3-DMA (KC3) LNPs containing different densities of αCD8 (TRX2 and 15C₀₁), at various dose levels, as illustrated by % DiI+ CD8+ T cells.

FIG. 37B depicts level of LNP association (Dil signal) in CD8+ T cells transfected with KC3 LNPs containing different densities of αCD8 (TRX2 and 15C01), at various dose levels, as illustrated by DiI MFI of CD8+ T cells.

FIG. 37C depicts level of GFP expression in CD8+ T cells transfected with KC3 LNPs containing different densities of αCD8 (TRX2 and 15C01), at various dose levels, as illustrated by % GFP+ CD8+ T cells.

FIG. 37D depicts level of GFP expression in CD8+ T cells transfected with KC3 LNPs containing different densities of αCD8 (TRX2 and 15C01), at various dose levels, as illustrated by GFP MFI of CD8+ T cells.

FIG. 37E depicts level of GFP expression in CD4+ T cells transfected with KC3 LNPs containing different densities of αCD8 (TRX2 and 15C01), at various dose levels, as illustrated by % GFP+ CD4+ T cells.

FIG. 37F depicts level of GFP expression in CD4+ T cells transfected with KC3 LNPs containing different densities of αCD8 (TRX2 and 15C01), at various dose levels, as illustrated by GFP MFI of CD4+ T cells.

FIG. 38A depicts viability of CD8+ T cells transfected with Lipid 15 LNPs containing different variants of the 15C01 αCD8 targeting moiety, at various densities and dose levels, as illustrated by % Live T cells.

FIG. 38B depicts LNP association levels (Dil signal) of CD8+ T cells transfected with Lipid 15 LNPs containing different variants of the 15C01 αCD8 targeting moiety, at various densities and dose levels, as illustrated by % DiI+ T cells.

FIG. 38C depicts LNP association levels (Dil signal) of CD8+ T cells transfected with Lipid 15 LNPs containing different variants of the 15C01 αCD8 targeting moiety, at various densities and dose levels, as illustrated by Dil MFI of T cells.

FIG. 38D depicts GFP expression levels of CD8+ T cells transfected with Lipid 15 LNPs containing different variants of the 15C01 αCD8 targeting moiety, at various densities and dose levels, as illustrated by % GFP+ T cells.

FIG. 38E depicts GFP expression levels of CD8+ T cells transfected with Lipid 15 LNPs containing different variants of the 15C01 αCD8 targeting moiety, at various densities and dose levels, as illustrated by GFP MFI of T cells.

FIG. 39A depicts CD69 expression levels of CD8+ T cells transfected with Lipid 15 LNPs containing αCD3 (SP34) or αCD8 (15C01v8 or TRX2) targeting moieties, at various dose levels, as illustrated by CD69 MFI of T cells.

FIG. 39B illustrates a histogram of CD69 expression levels of CD8+ T cells transfected with Lipid 15 LNPs containing αCD3 (SP34) or αCD8 (15C01v8 or TRX2) targeting moieties, at a dose of 1 μg/mL mRNA.

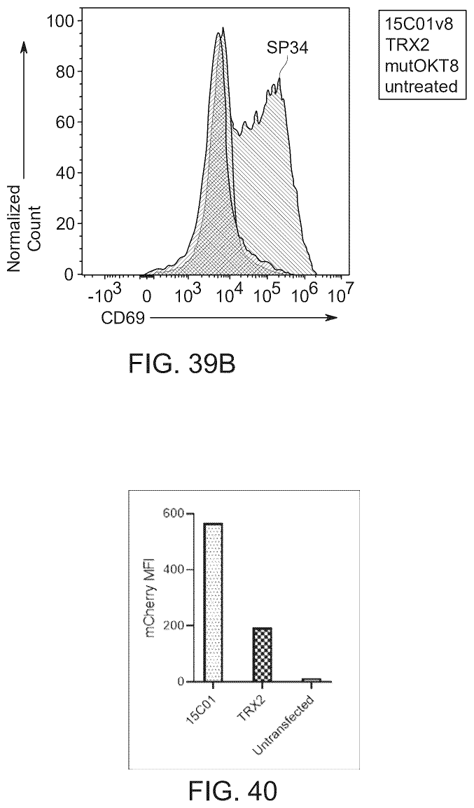

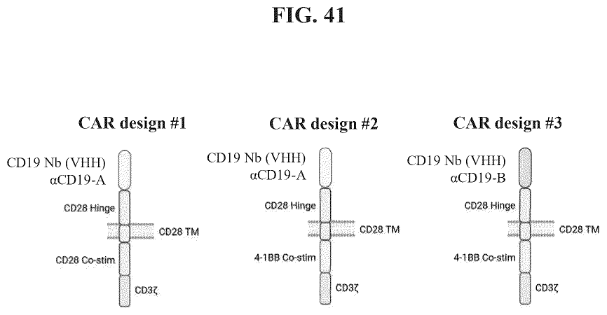

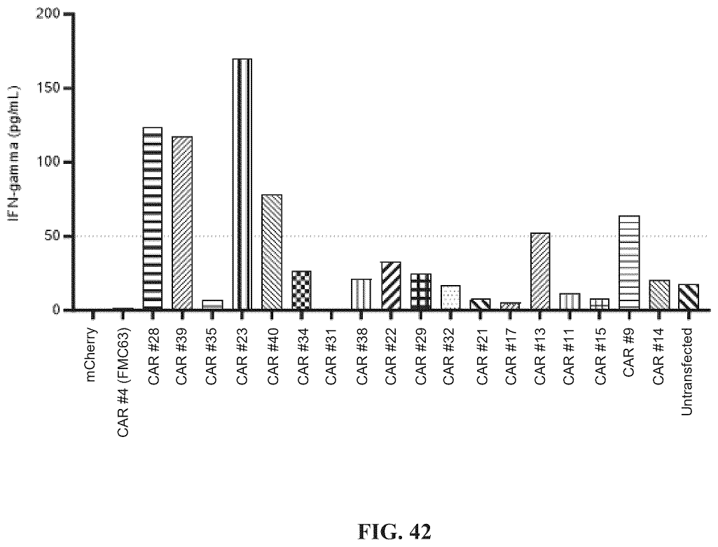

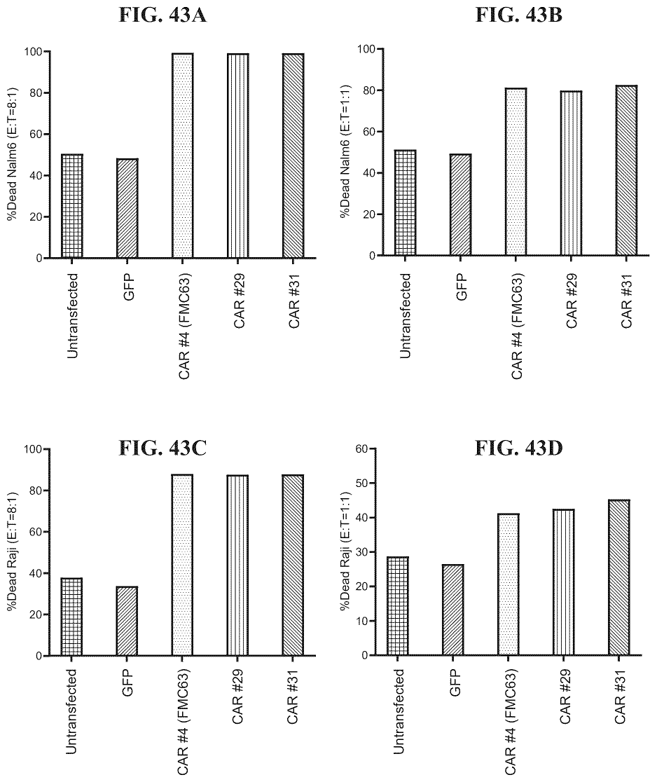

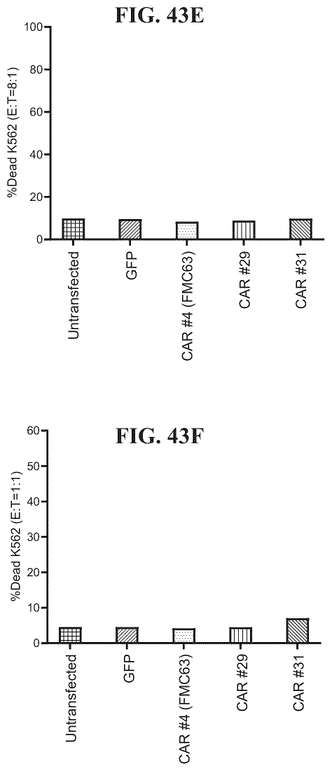

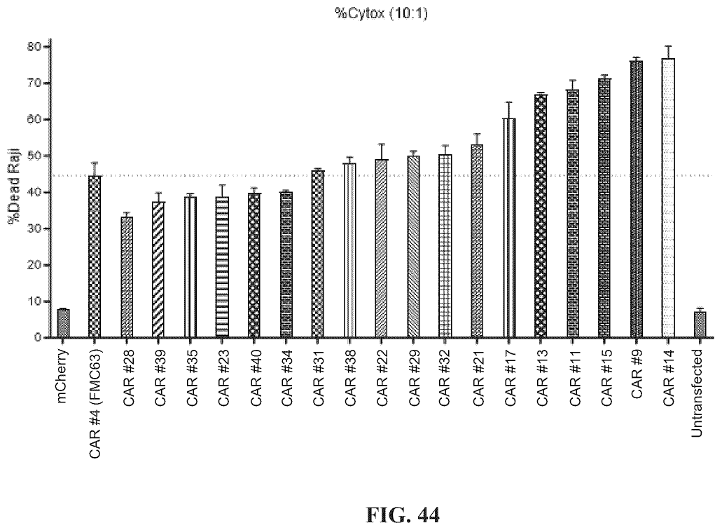

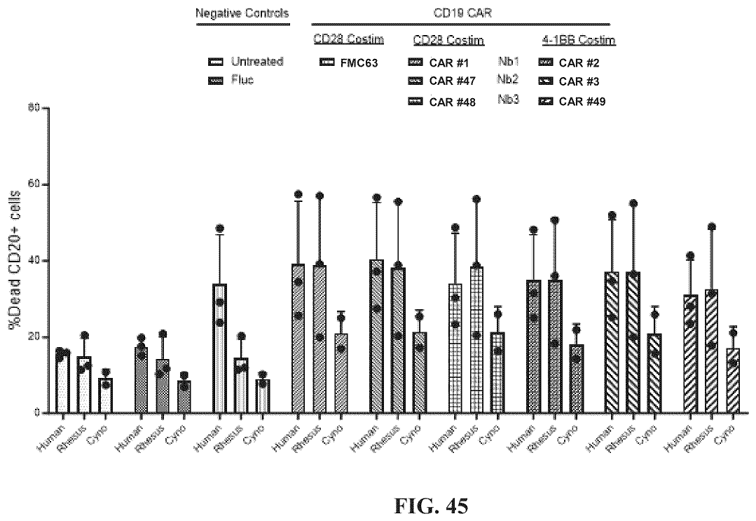

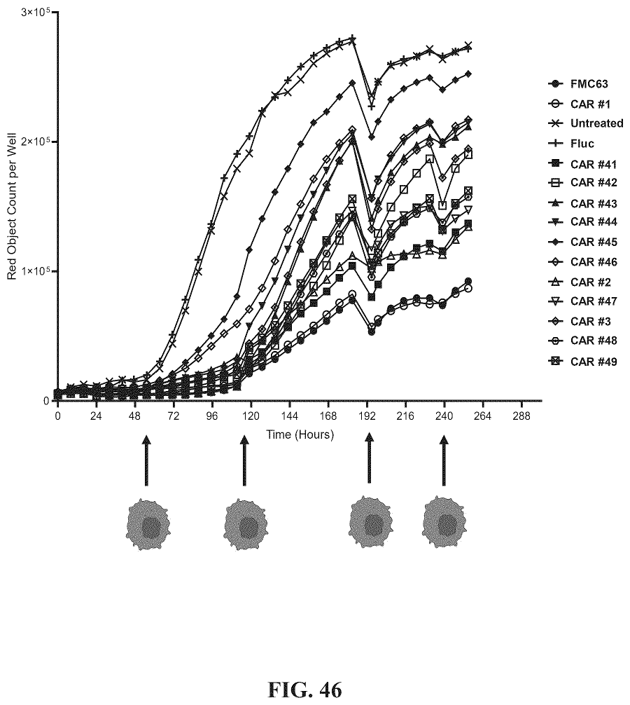

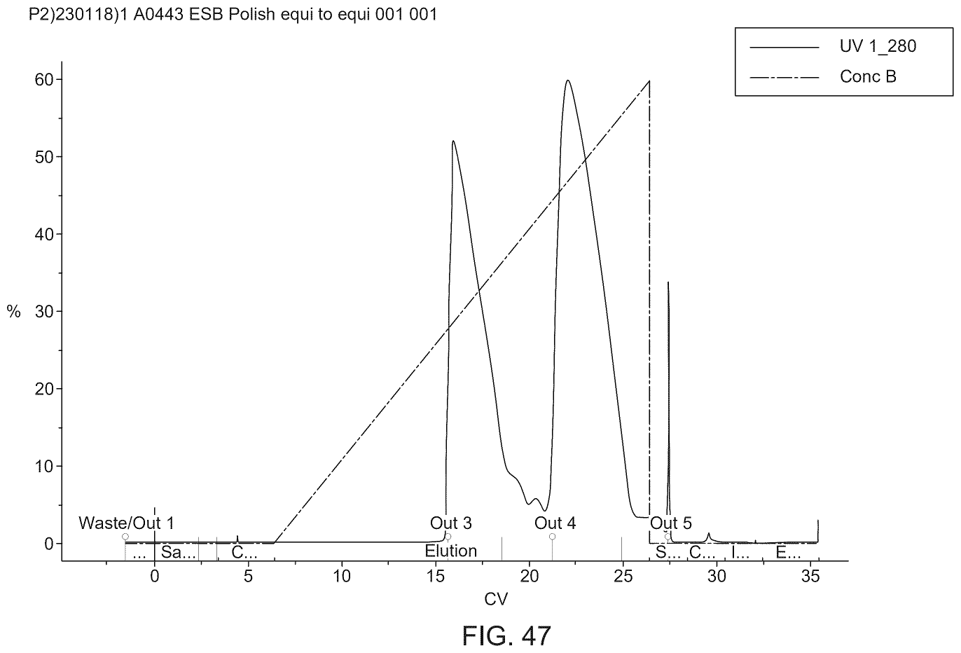

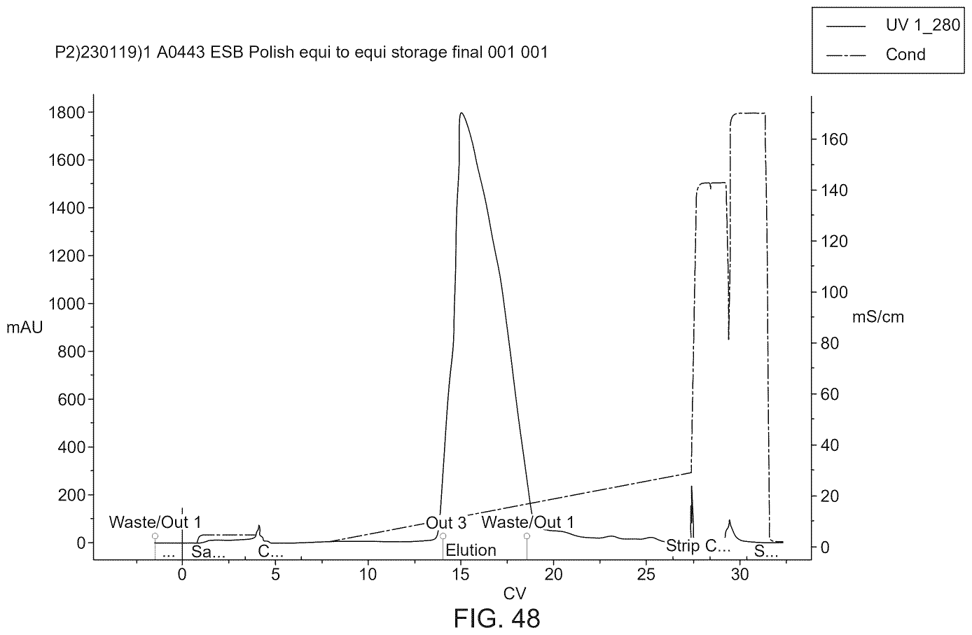

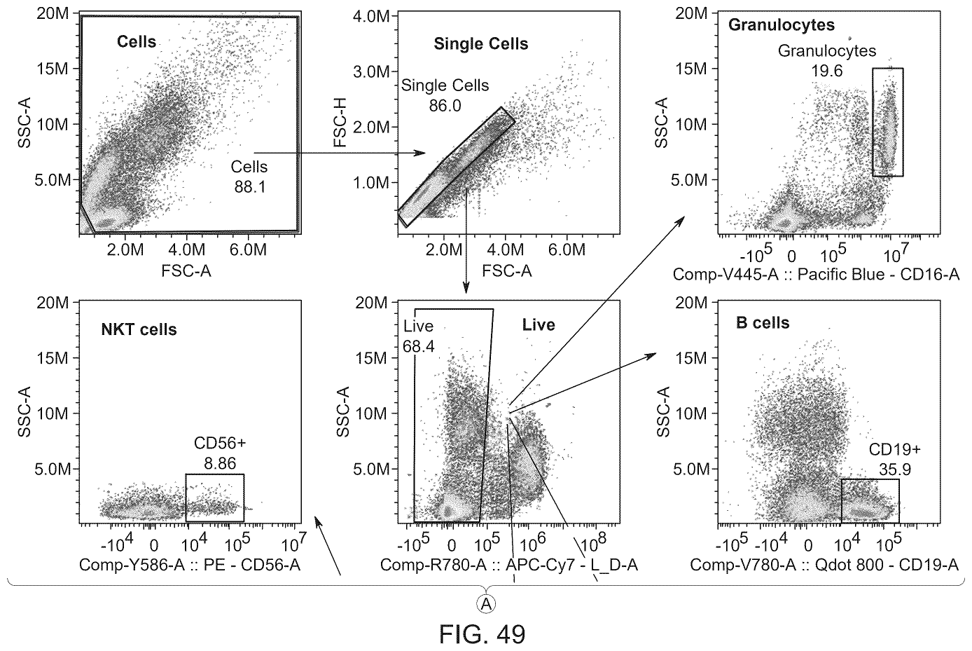

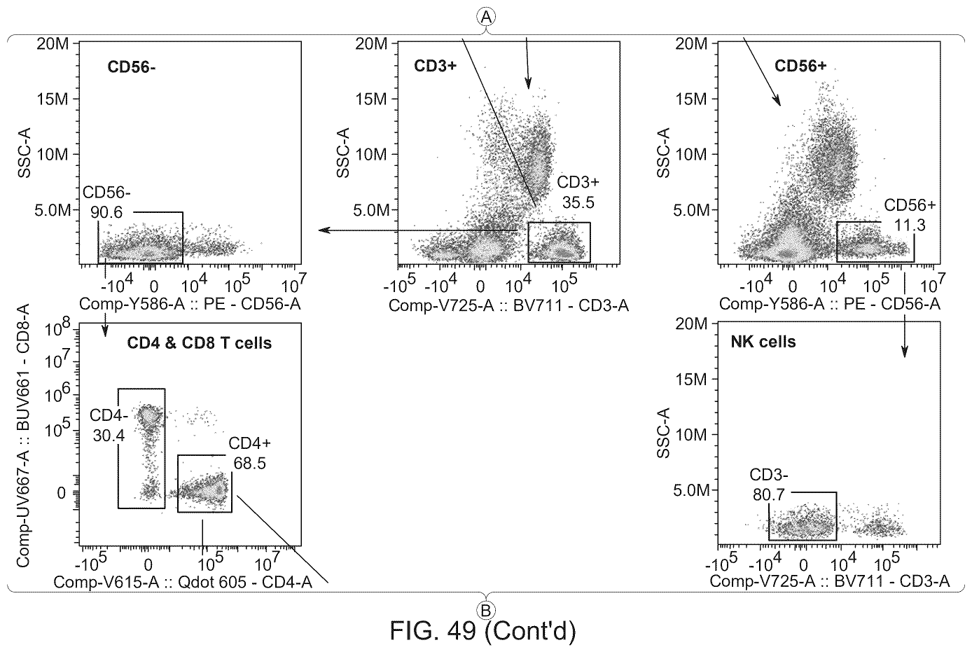

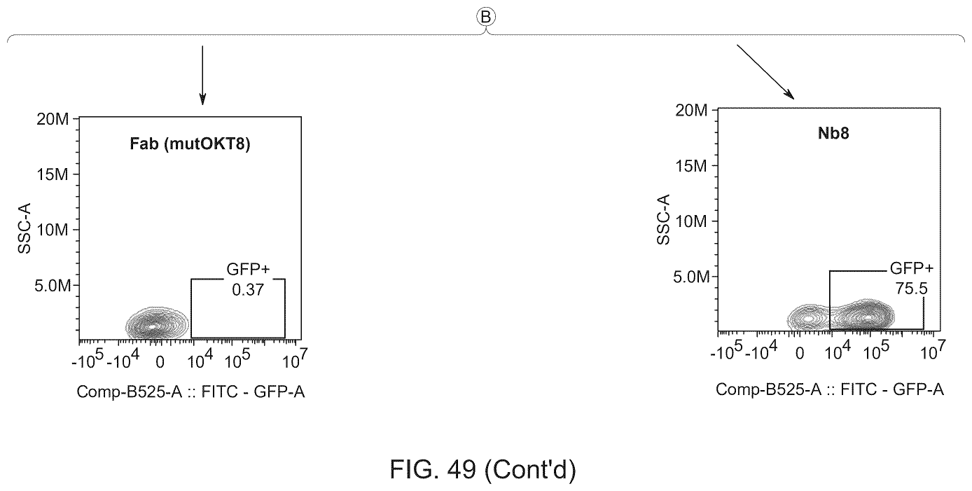

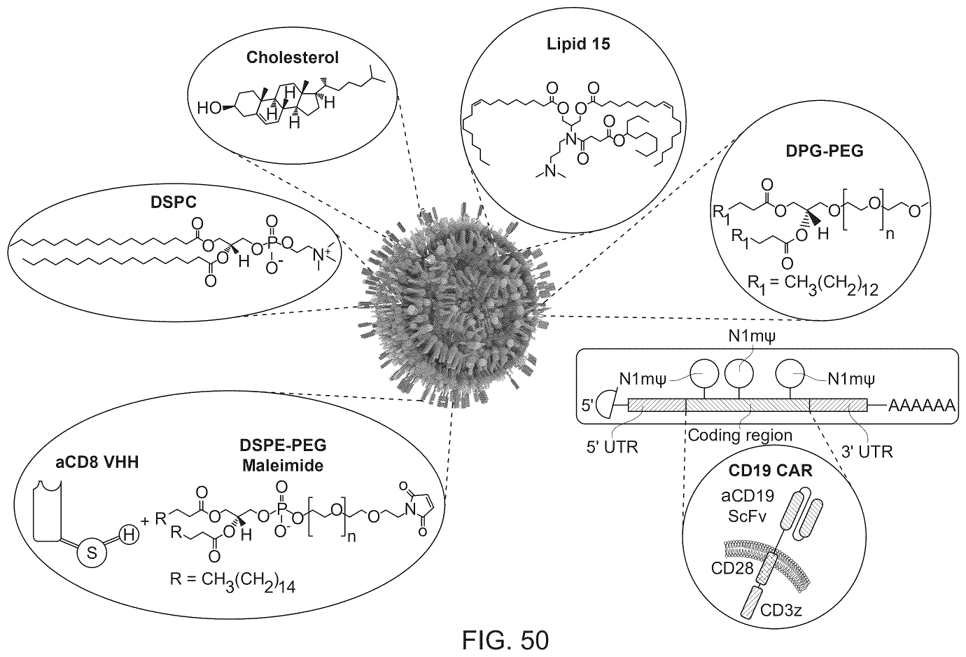

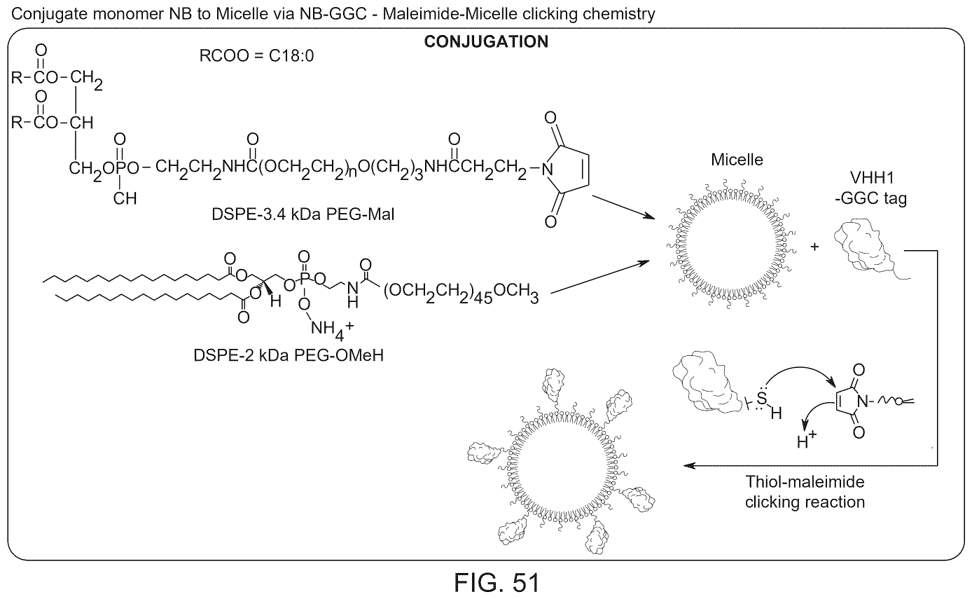

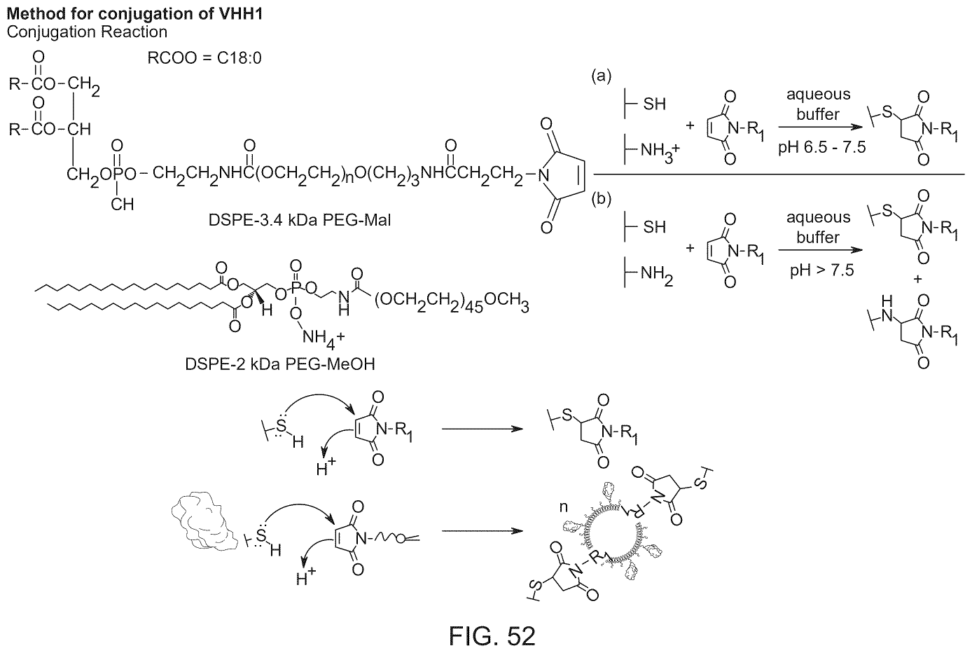

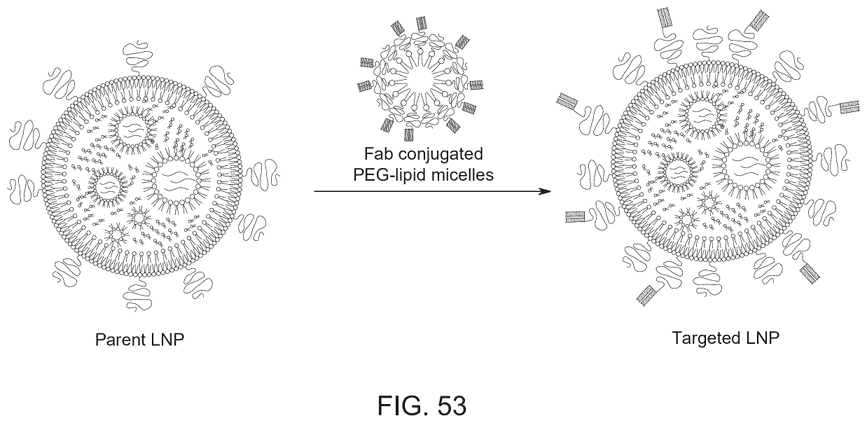

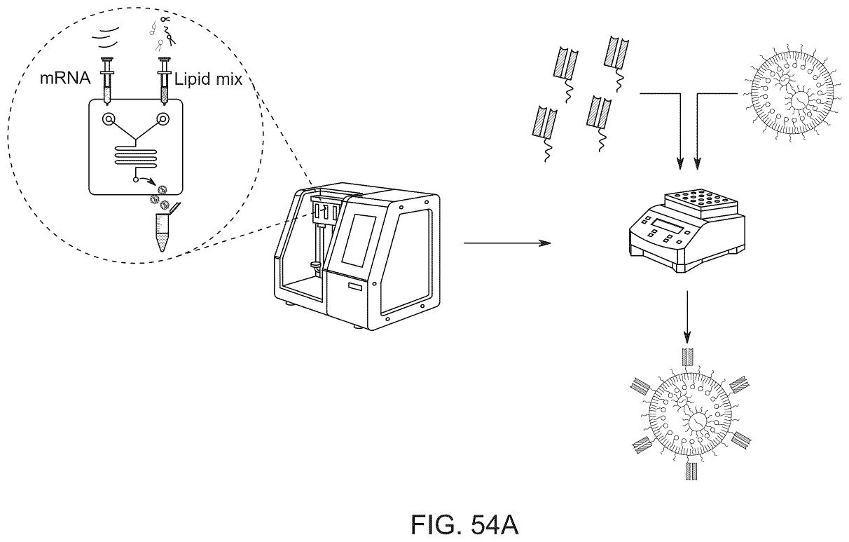

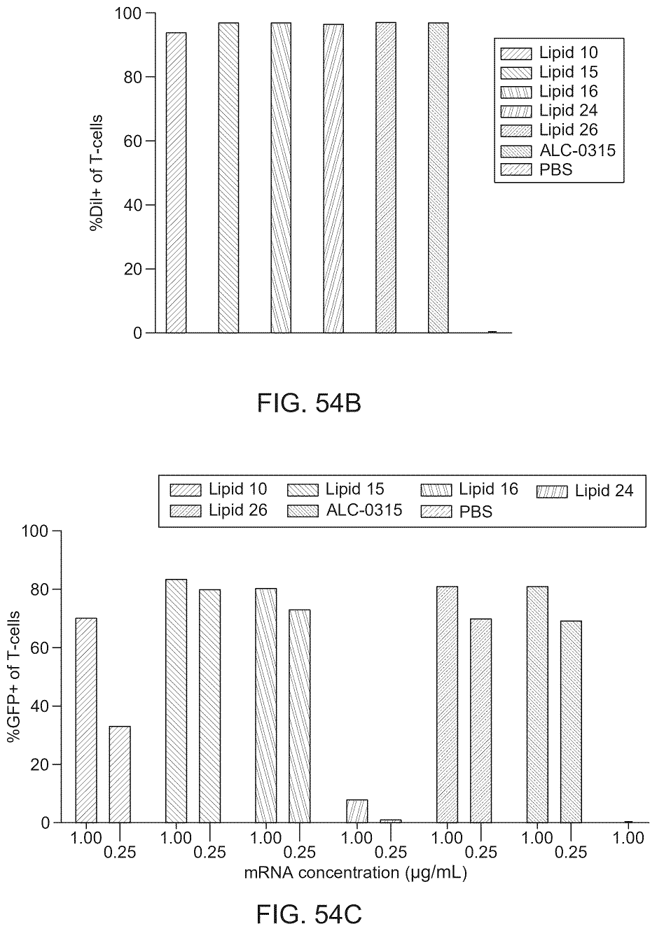

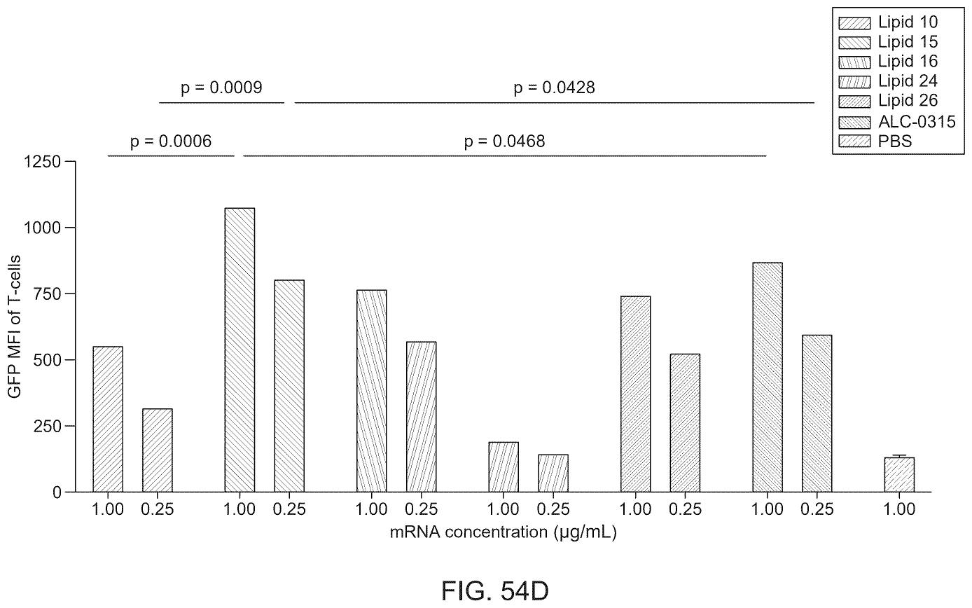

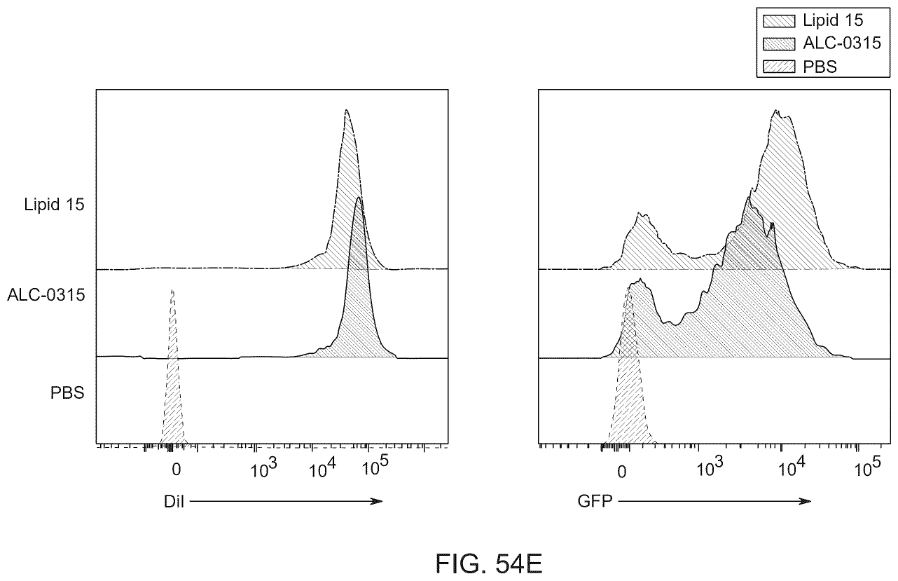

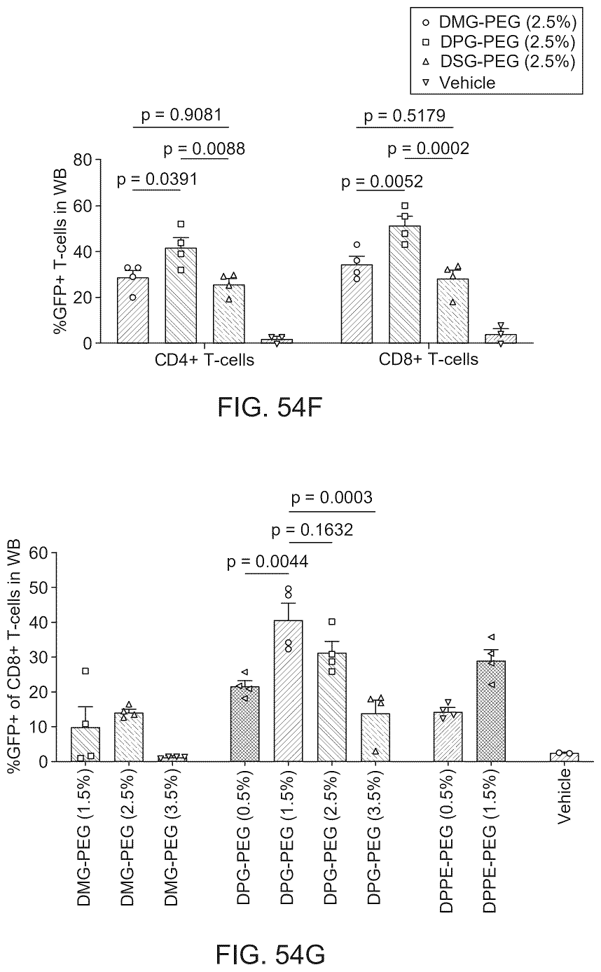

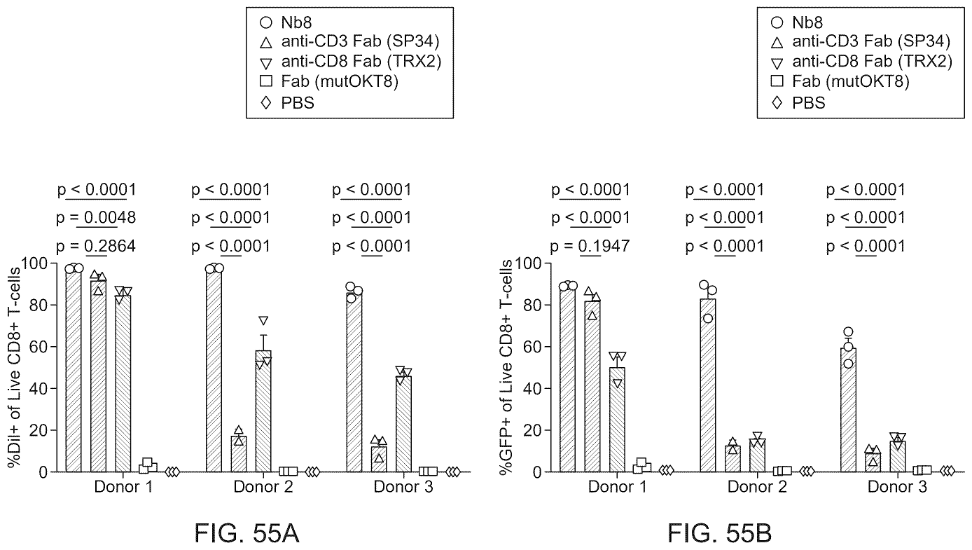

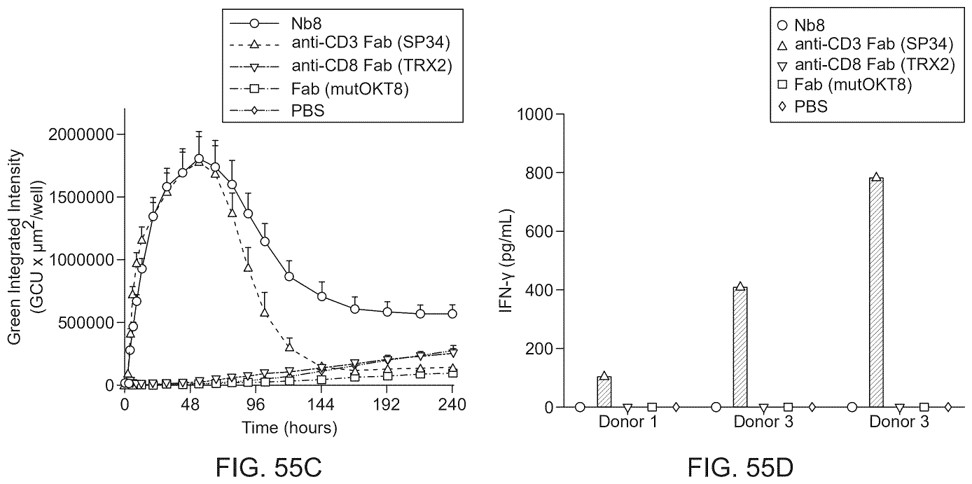

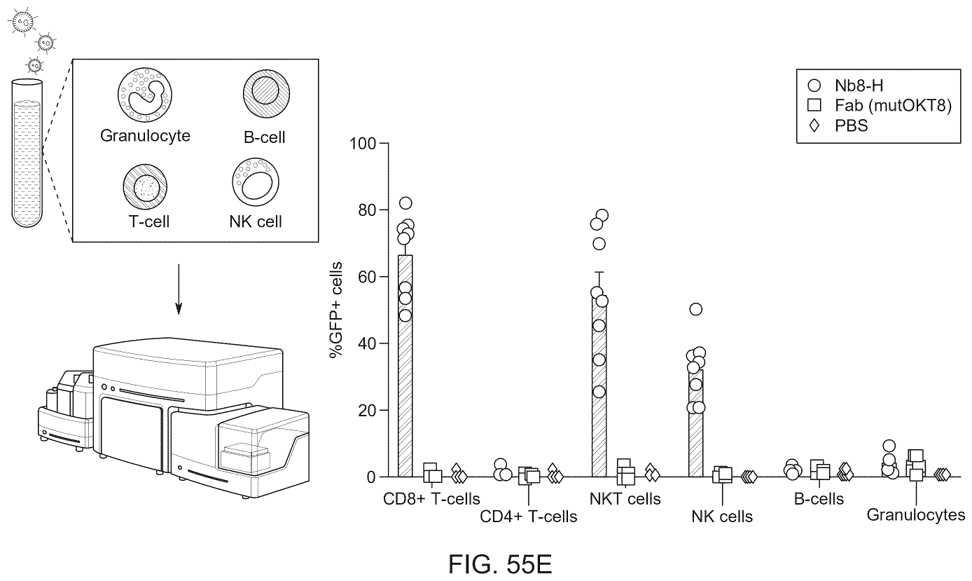

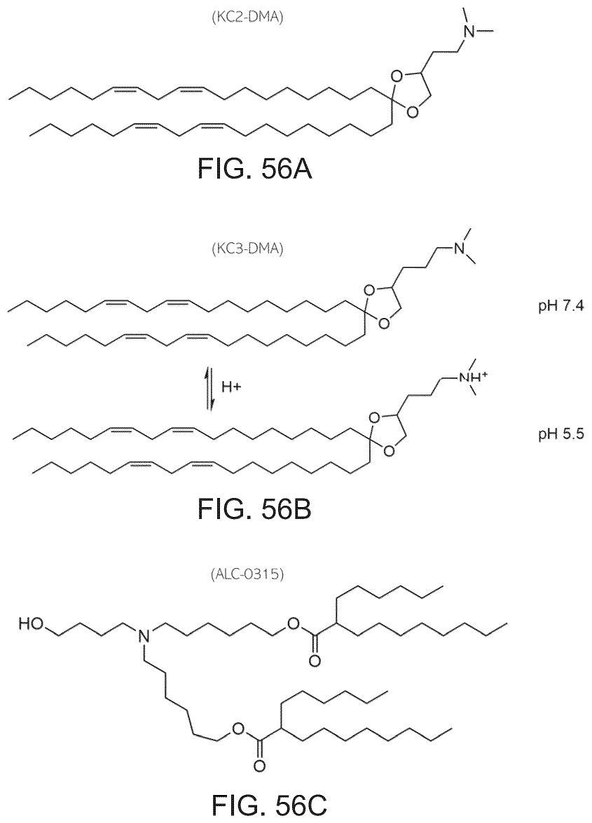

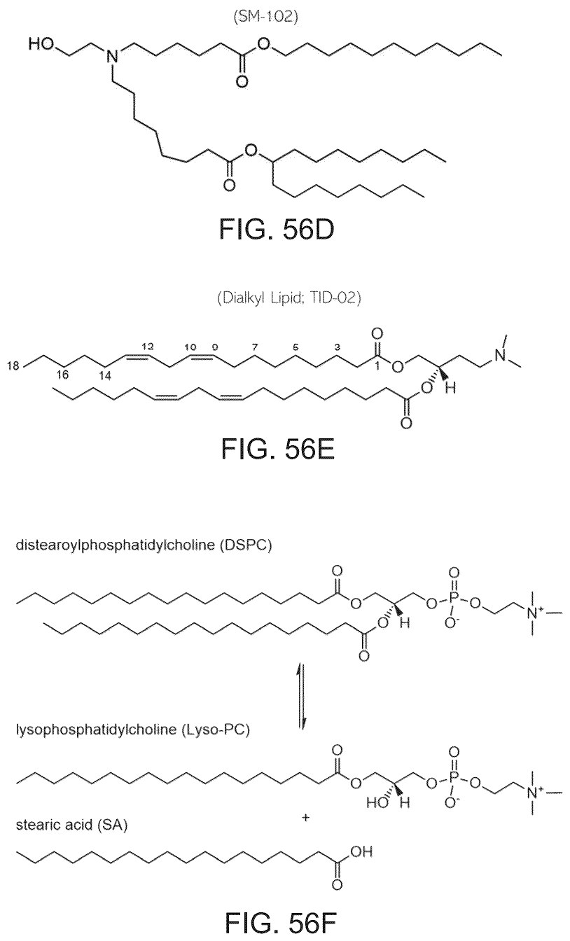

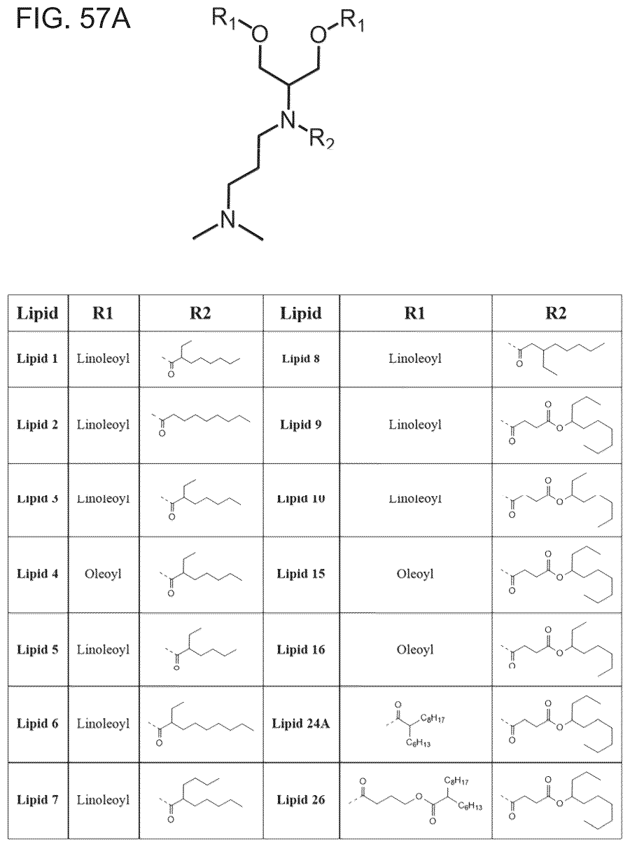

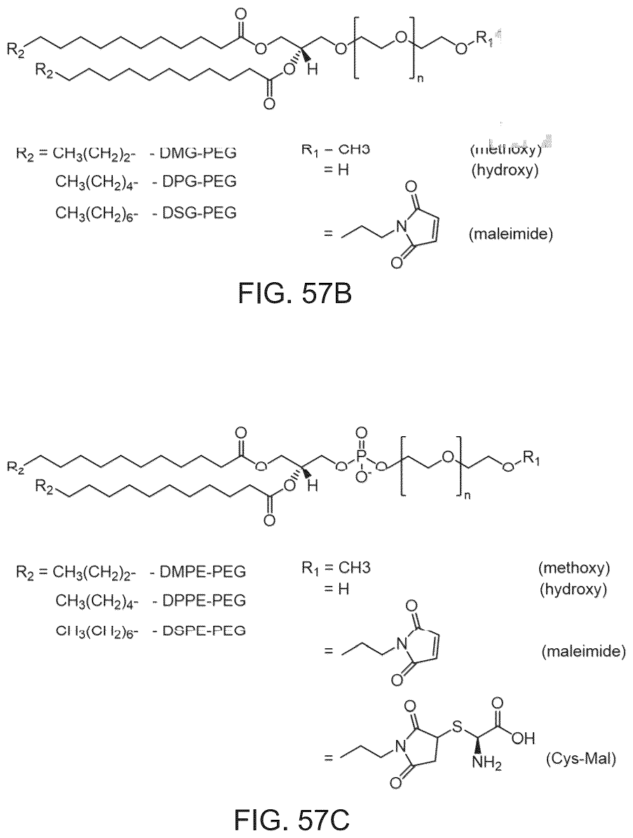

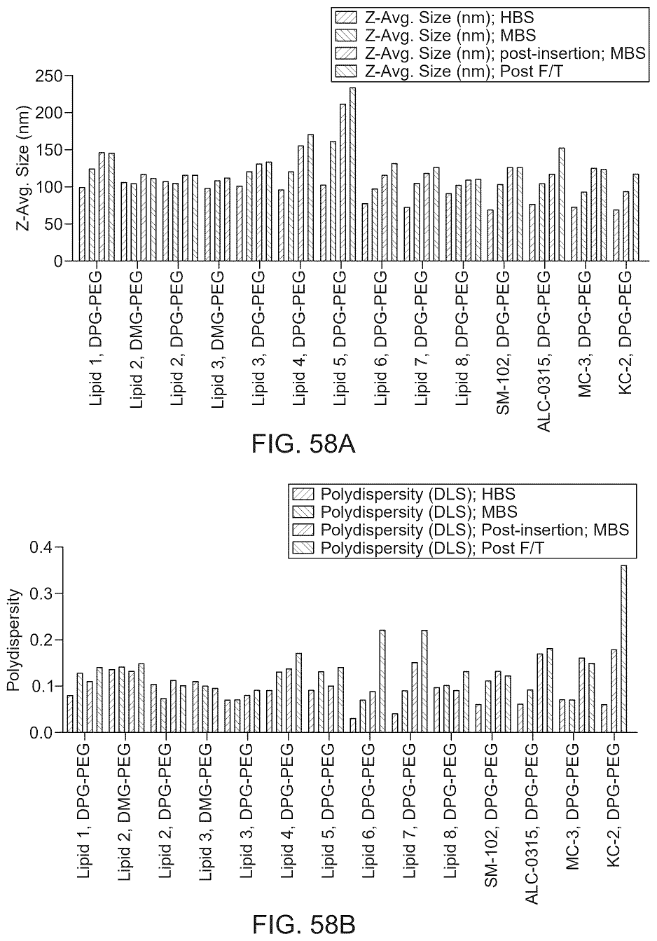

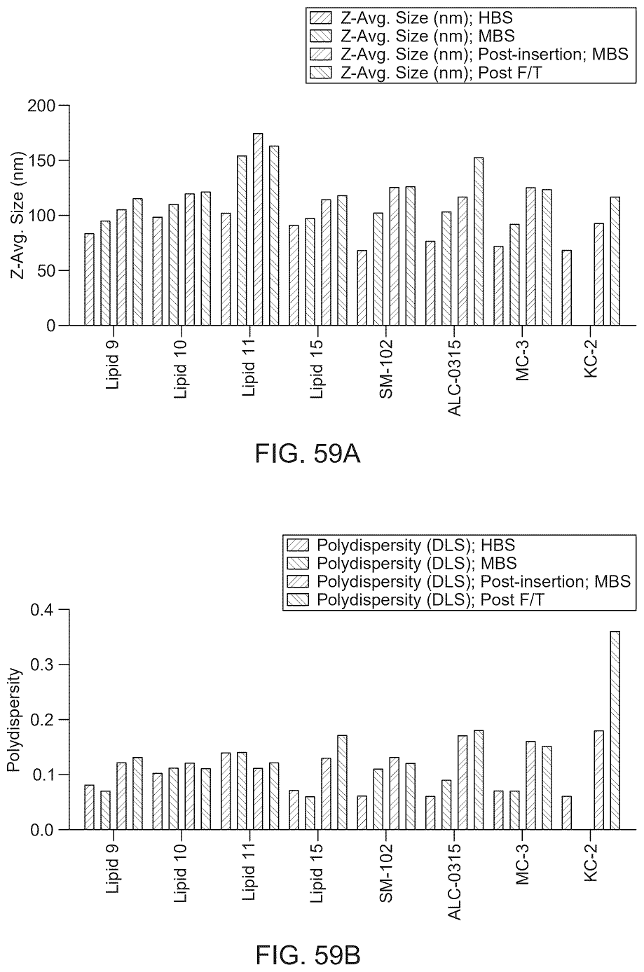

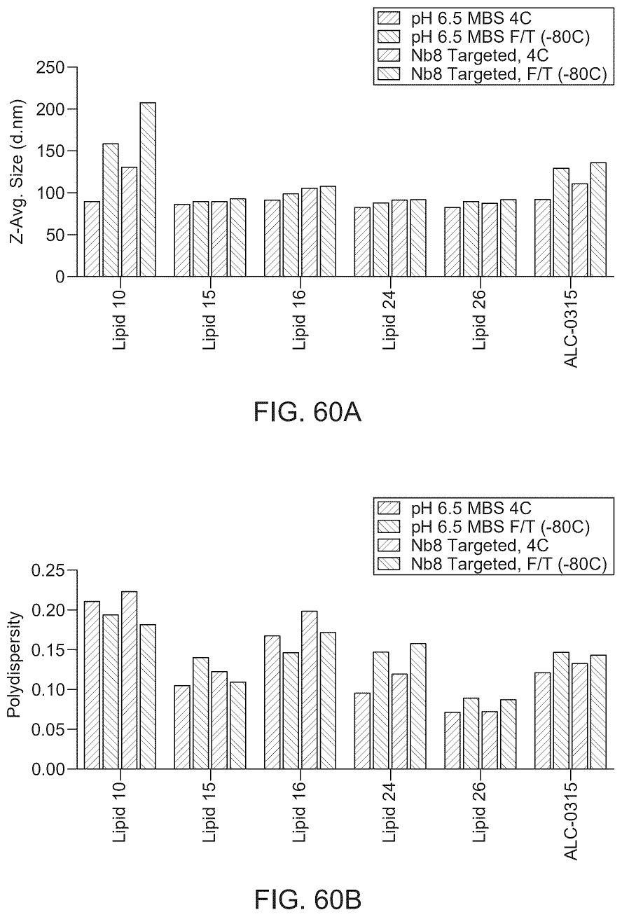

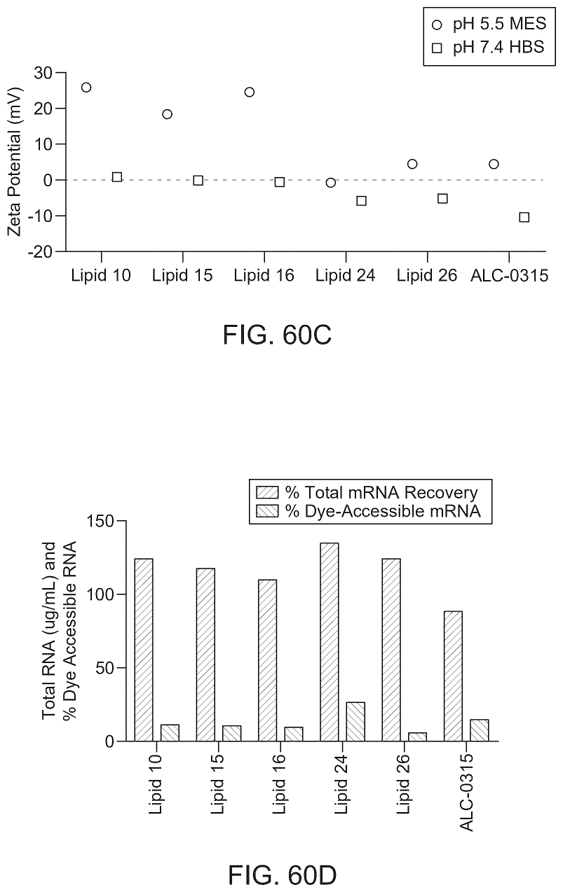

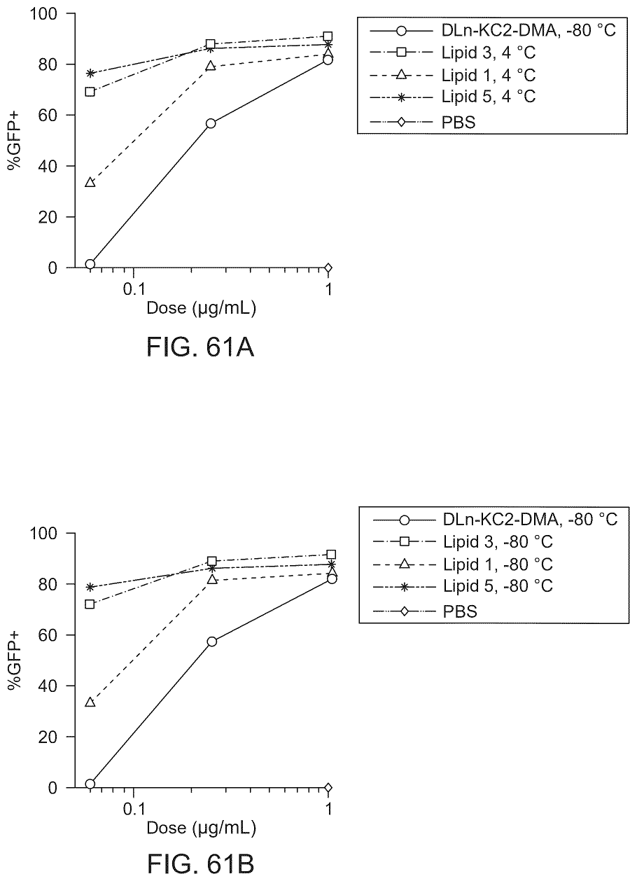

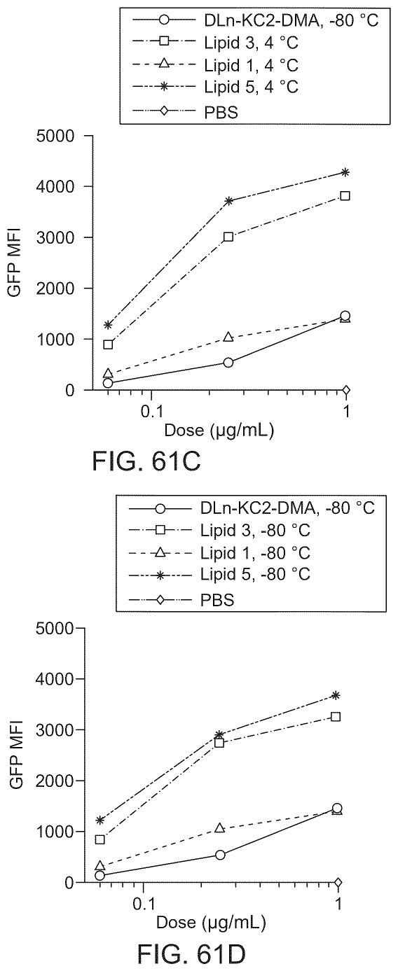

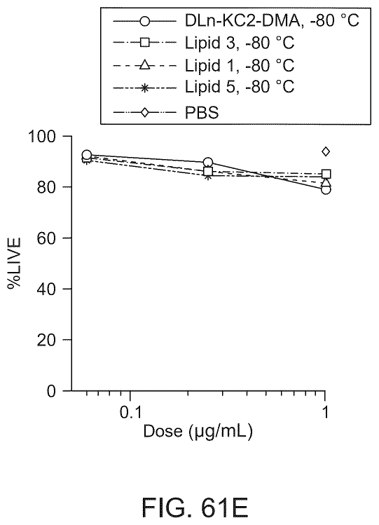

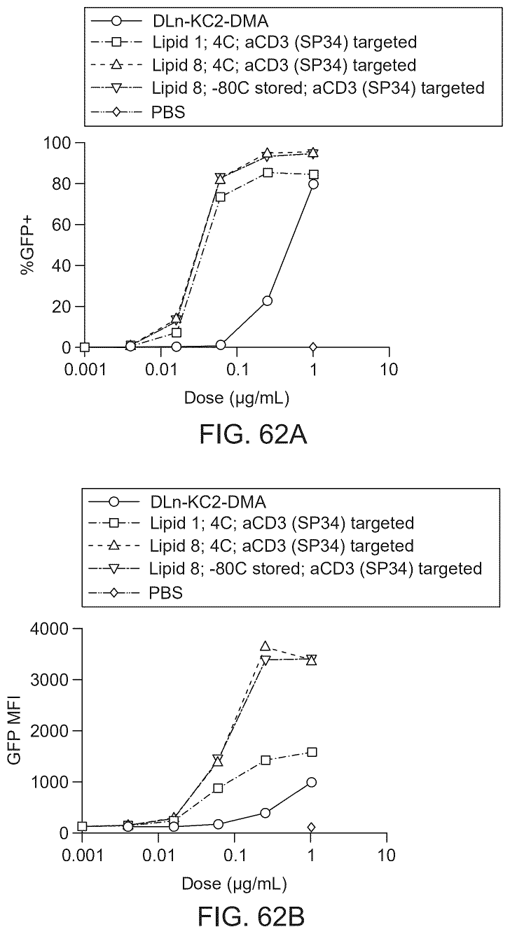

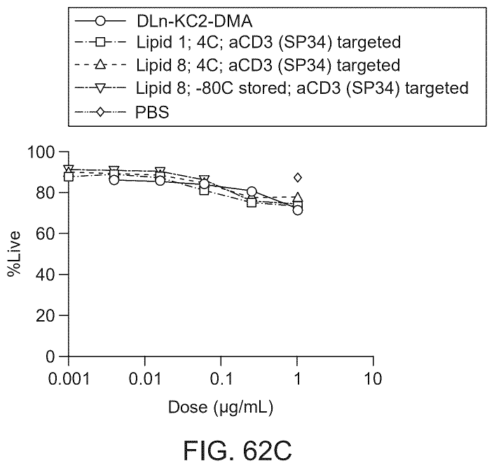

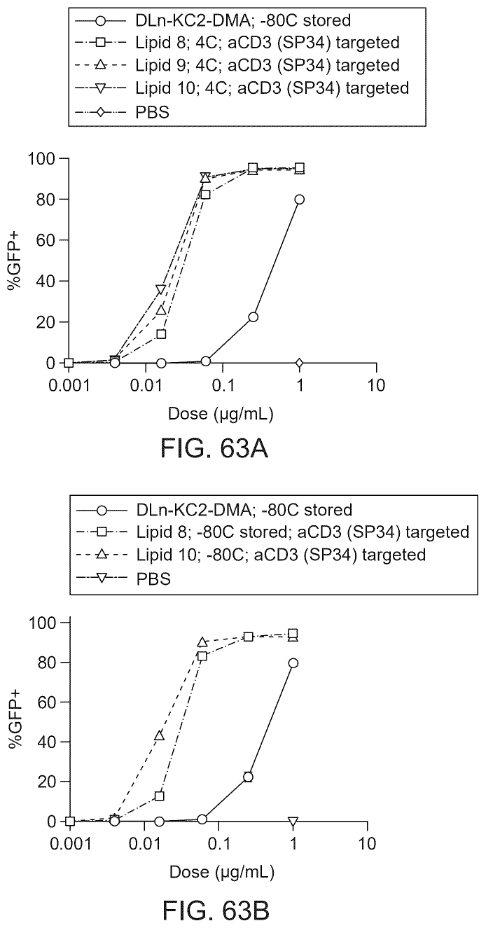

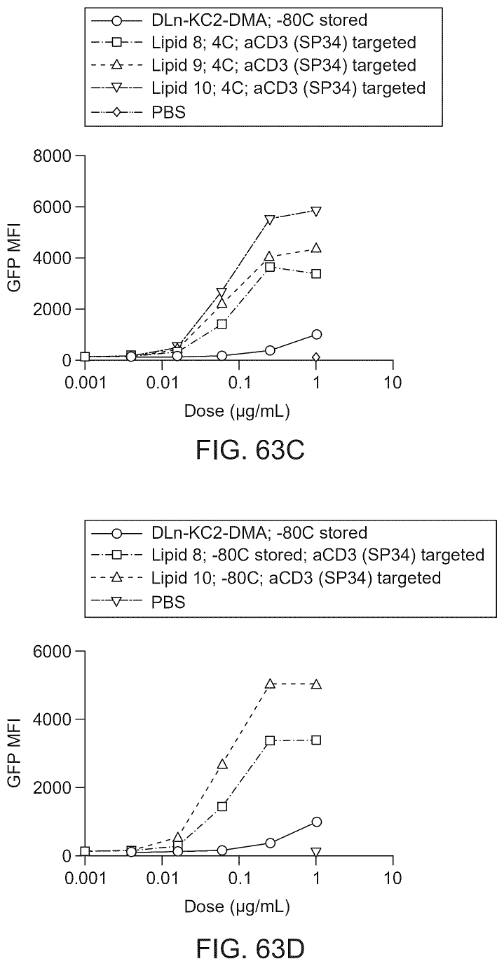

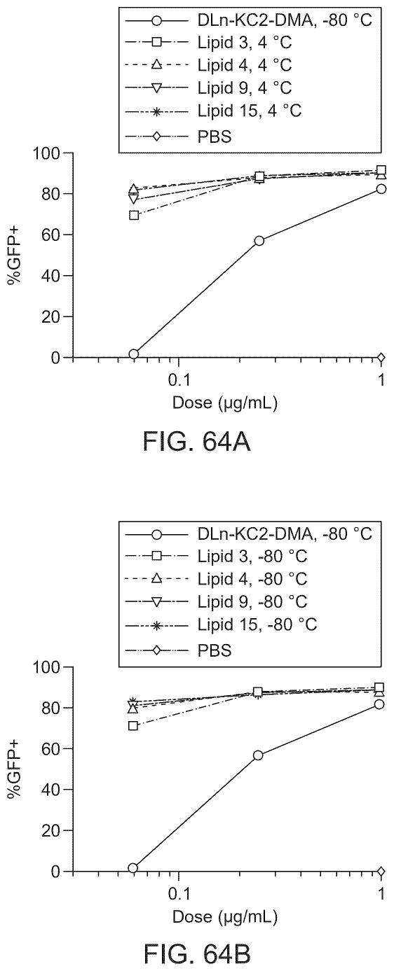

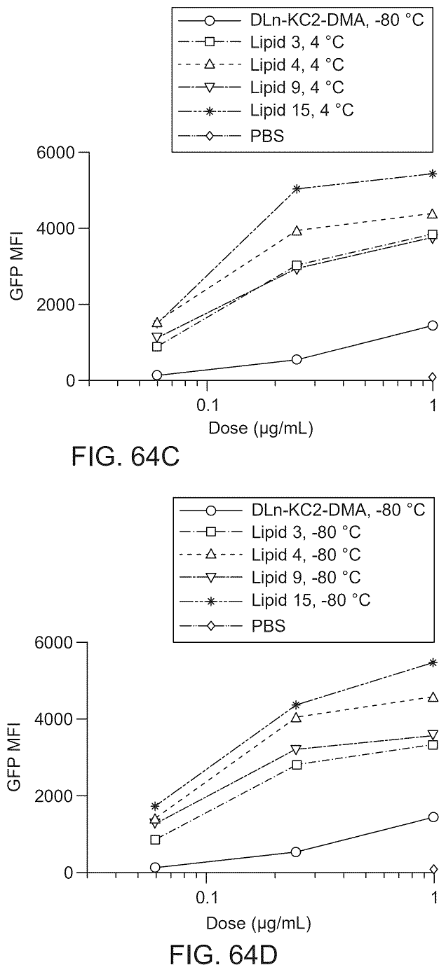

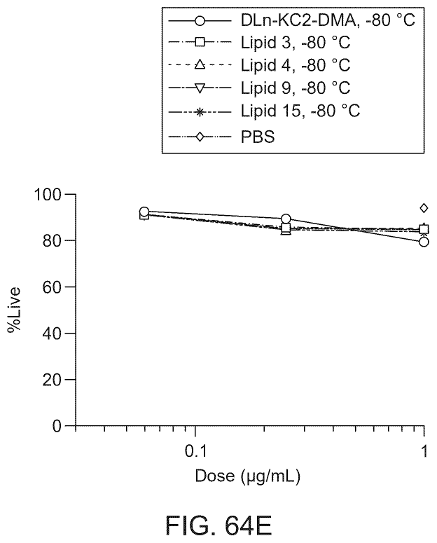

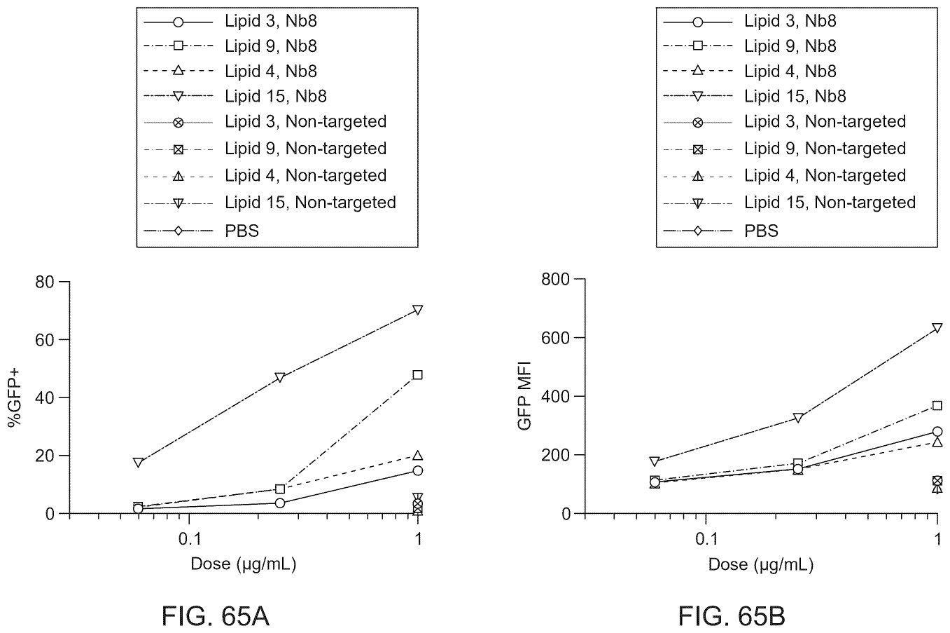

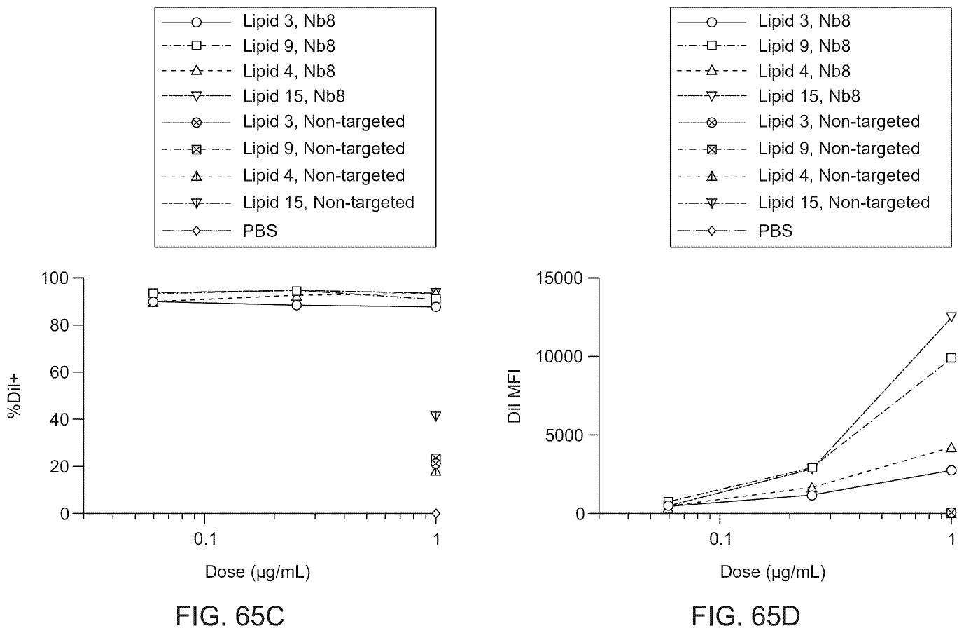

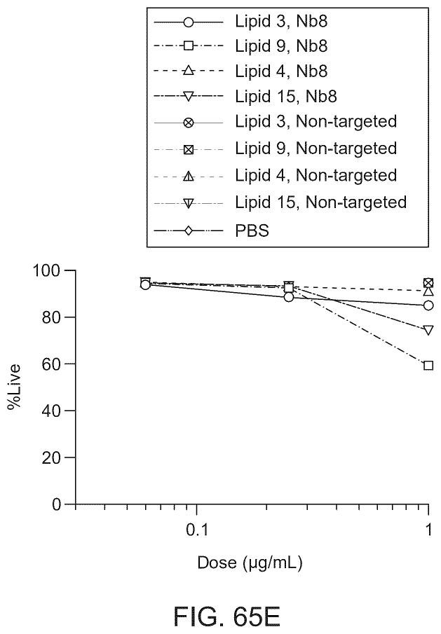

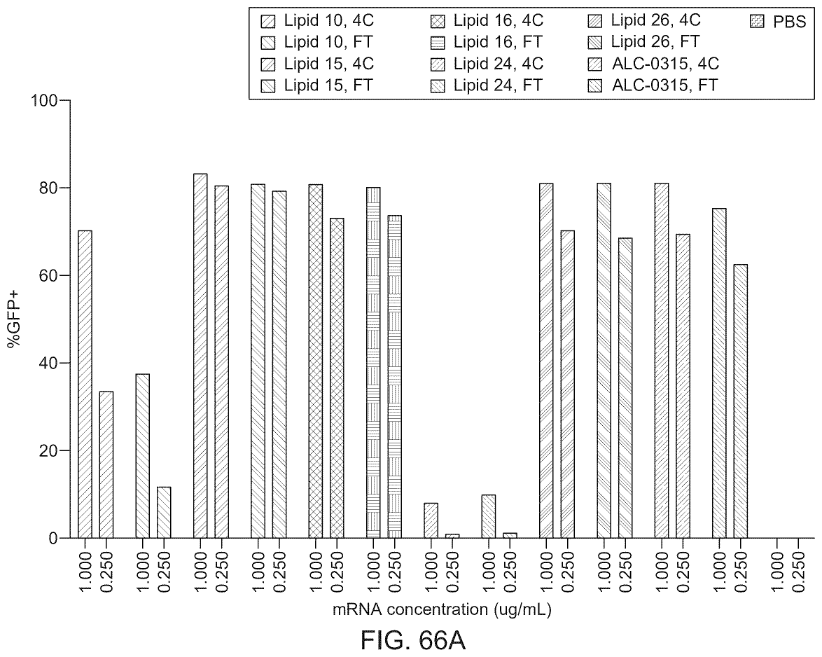

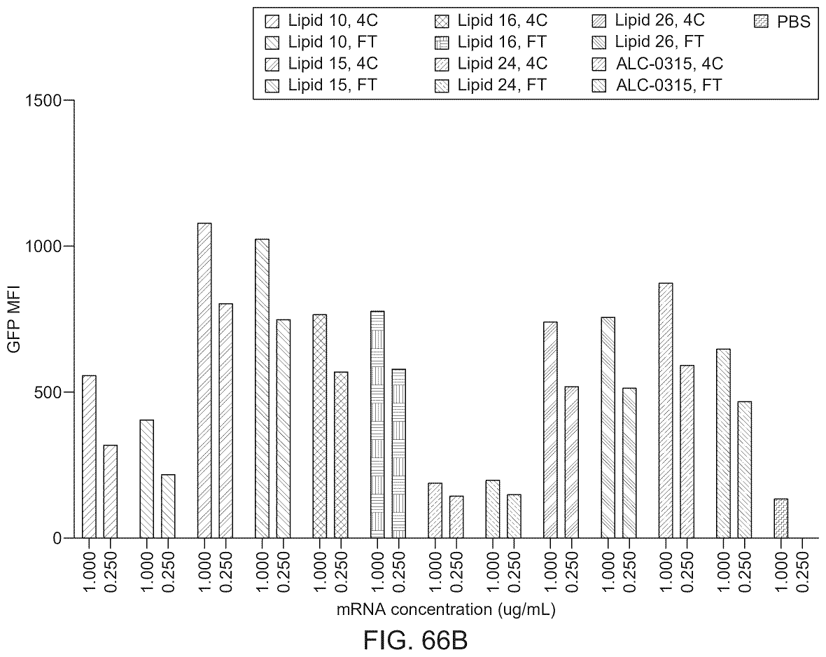

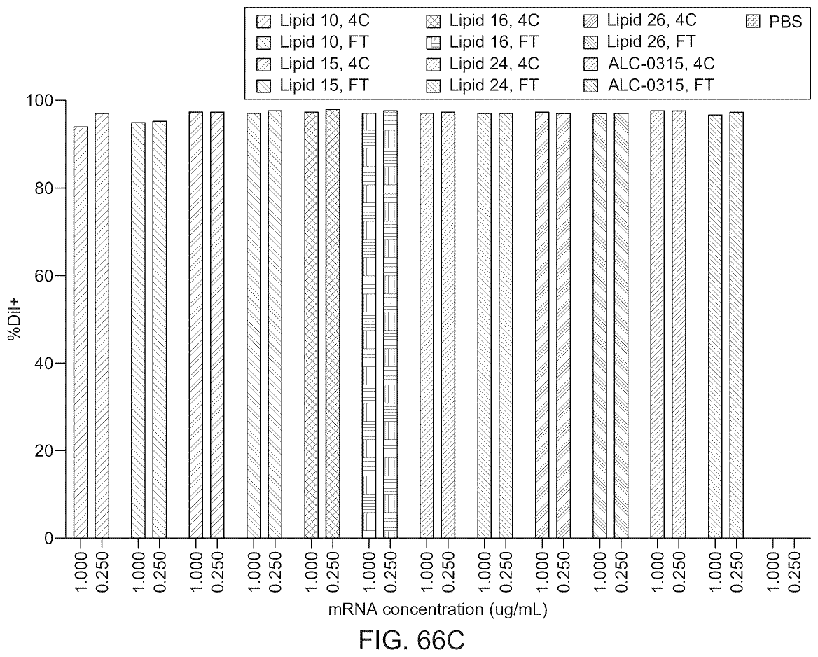

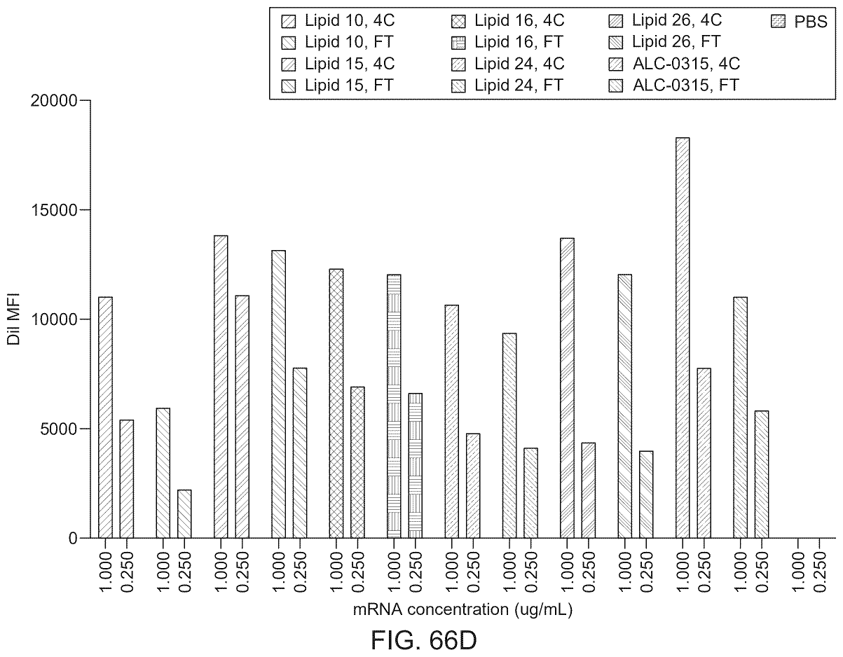

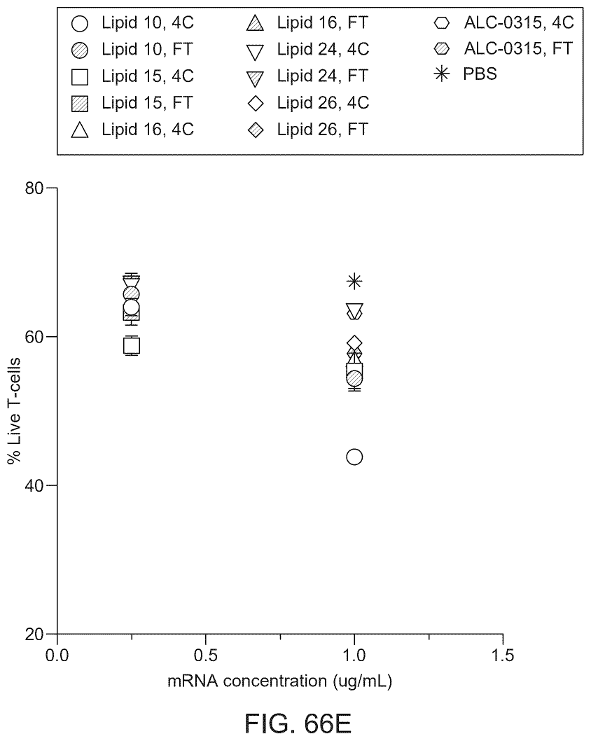

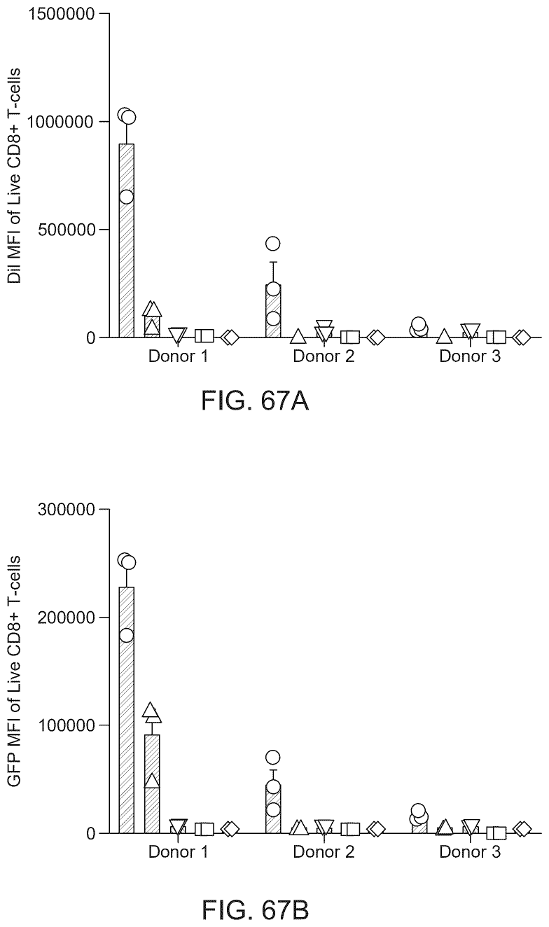

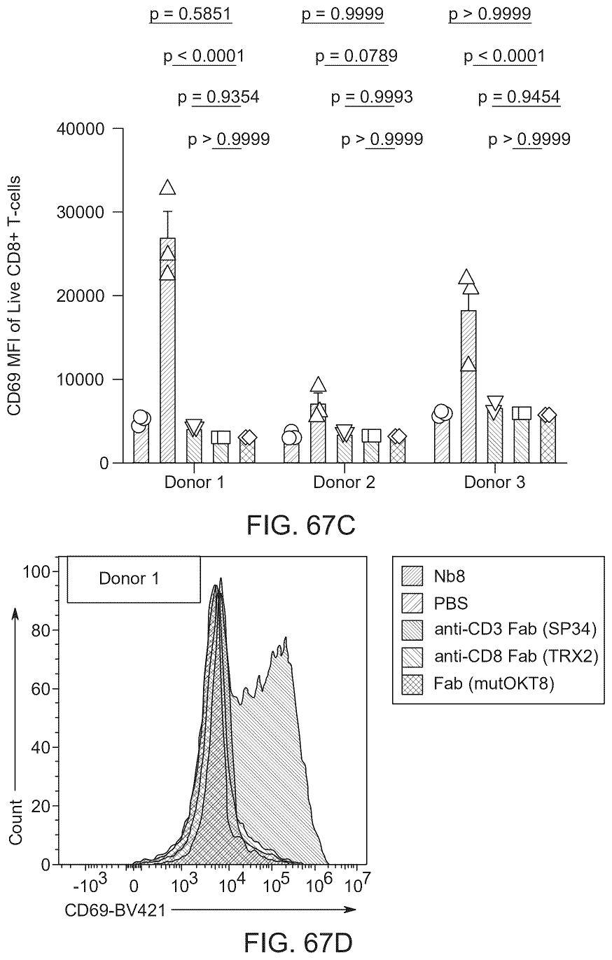

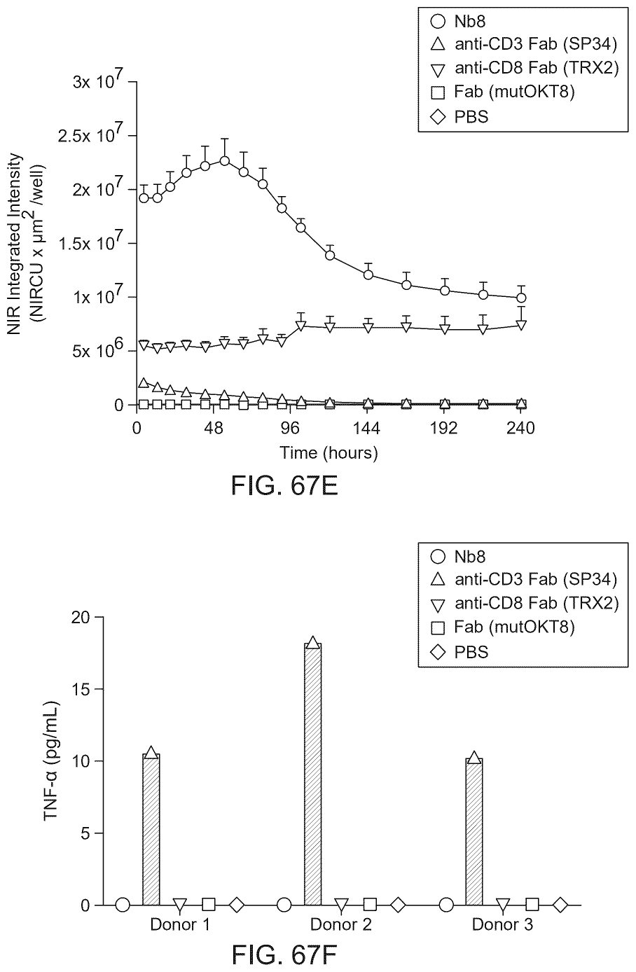

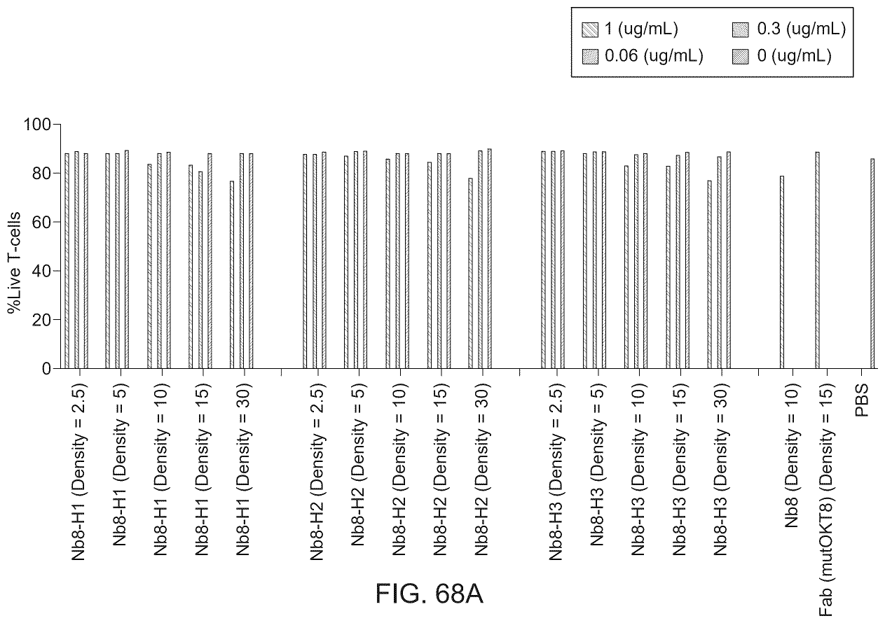

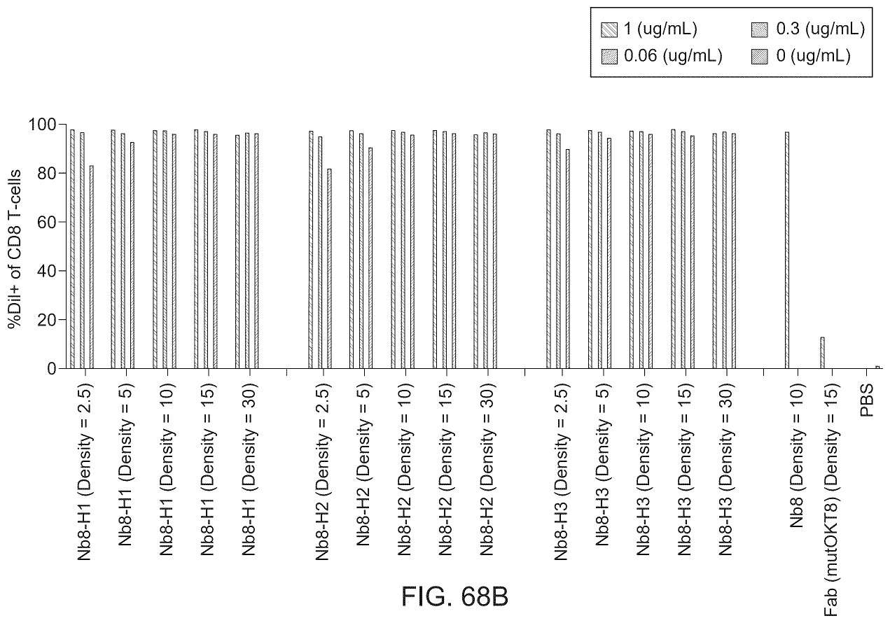

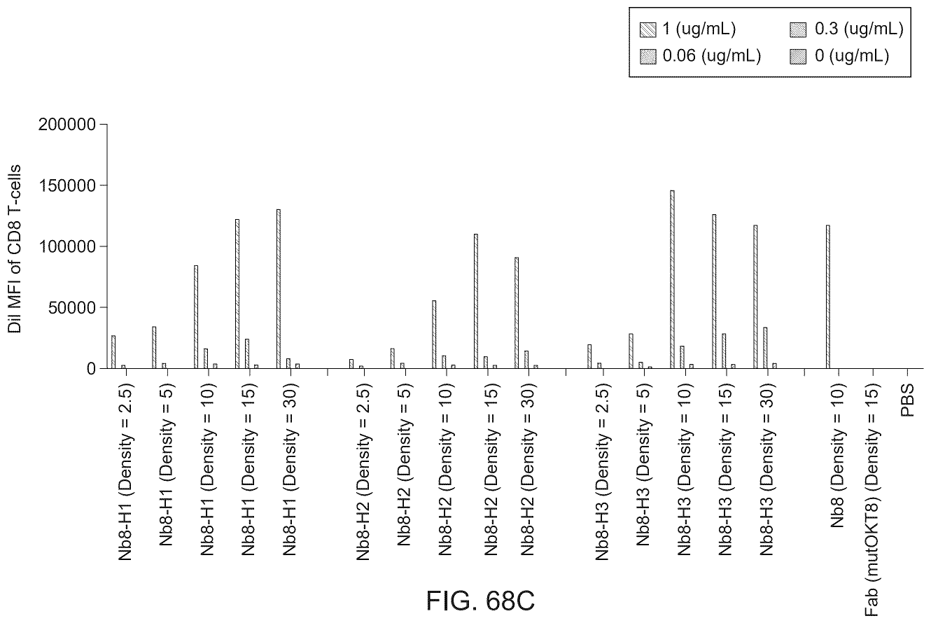

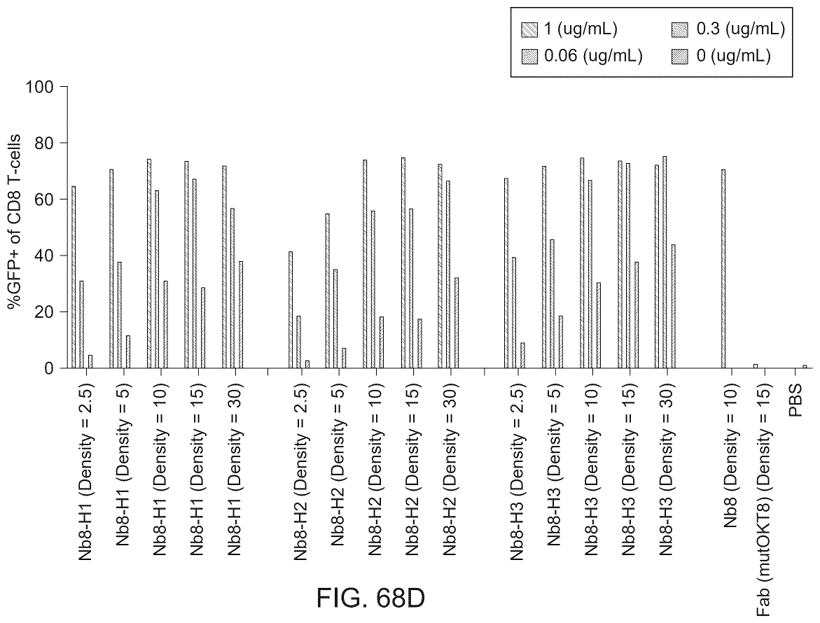

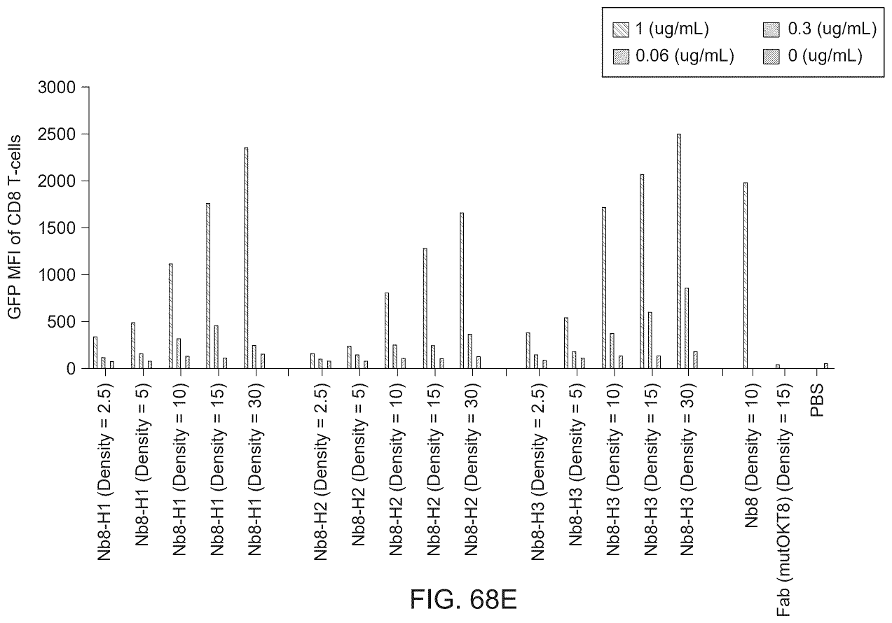

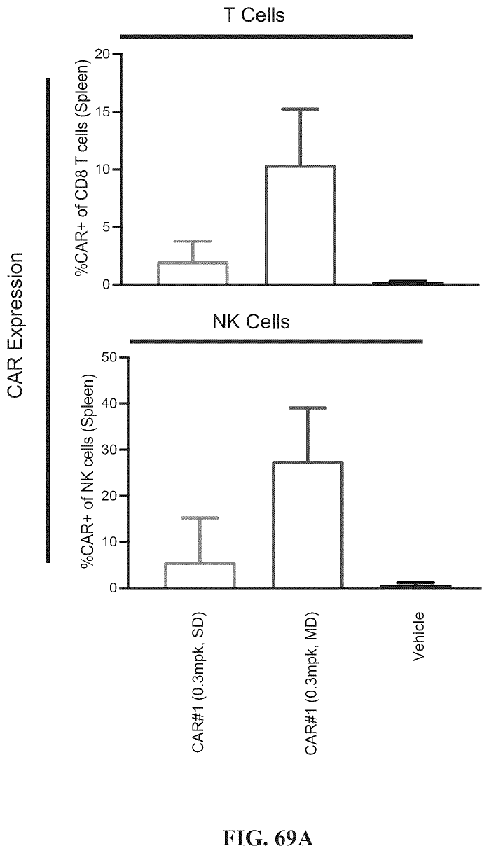

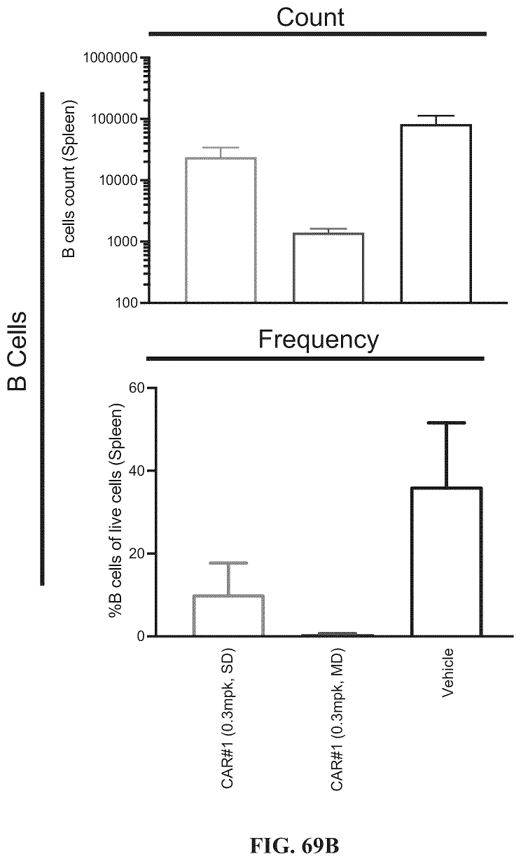

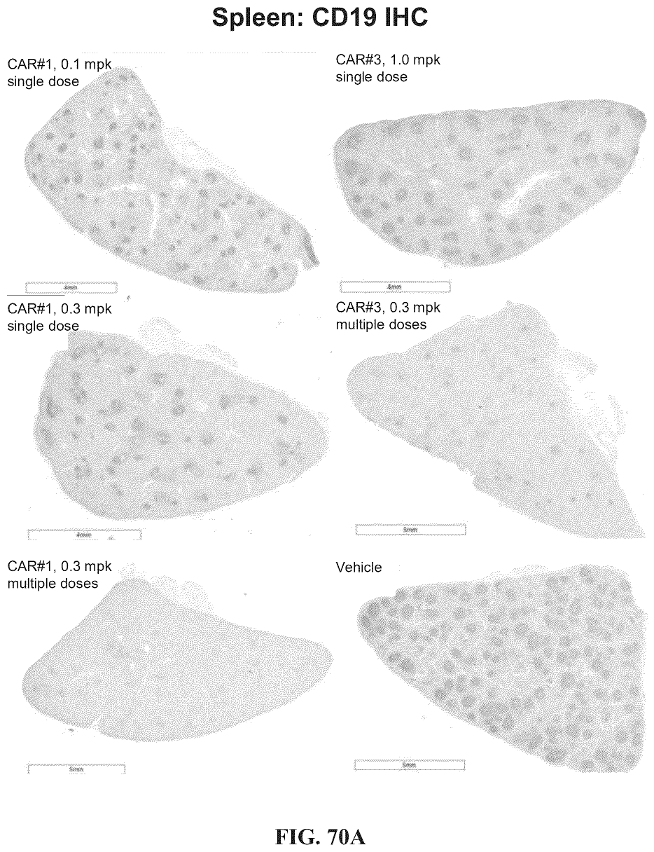

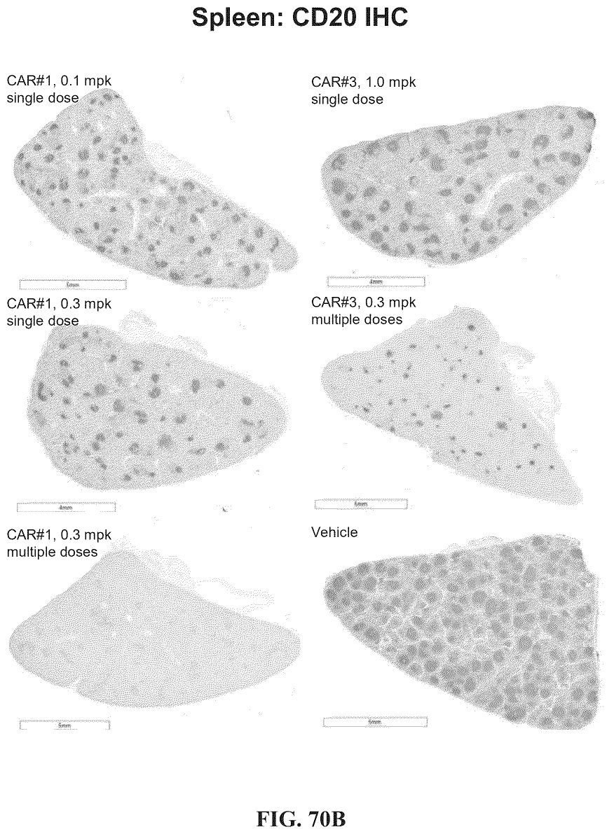

FIG. 40 depicts mCherry expression levels of primary NHP CD8+ T cells transfected with Lipid 15 LNPs containing different αCD8 targeting moieties (15C01 and TRX2) as illustrated by mCherry MFI of T cells.

FIG. 41 illustrates the general CAR design (top panel) consisting of an anti-CD19 VHH domain connected by a linker, an extracellular hinge domain, a transmembrane domain, and an intracellular co-stimulatory domain and signaling domain. The bottom panel illustrates three different CAR cassette designs with varying intracellular co-stimulatory and/or anti-CD19 VHH domains.

FIG. 42 shows the comparison of IFN-γ secretion in primary human CD8+ T cells electroporated with exemplary anti-CD19 CAR constructs and assessed by IFN-γ ELISA.

FIGS. 43A-43F show the levels of anti-CD19 CAR-mediated cytotoxicity of CD8+ T cells transfected with CAR-comprising LNPs against target cells at effector-to-target cell ratios (E:Ts) of 8:1 or 1:1. FIG. 43A shows the levels of anti-CD19 CAR-mediated cytotoxicity against wild-type Nalm6 cells at effector-to-target cell ratios (E:Ts) of 8:1. FIG. 43B shows the levels of anti-CD19 CAR-mediated cytotoxicity against wild-type Nalm6 cells at effector-to-target cell ratios (E:Ts) of 1:1. FIG. 43C shows the levels of anti-CD19 CAR-mediated cytotoxicity against wild-type Raji cells at effector-to-target cell ratios (E:Ts) of 8:1. FIG. 43D shows the levels of anti-CD19 CAR-mediated cytotoxicity against wild-type Raji cells at effector-to-target cell ratios (E:Ts) of 1:1. FIG. 43E shows the levels of anti-CD19 CAR-mediated cytotoxicity against K562 cells at effector-to-target cell ratios (E:Ts) of 8:1. FIG. 43F shows the levels of anti-CD19 CAR-mediated cytotoxicity against K562 cells at effector-to-target cell ratios (E:Ts) of 1:1.

FIG. 44 shows the level of anti-CD19 CAR-mediated cytotoxicity of Raji cells resulting from anti-CD19 CAR mRNA transfection by electroporation and subsequent co-culture with CD19-expressing Raji cells. FMC63 was used as a benchmark control.

FIG. 45 shows the level of B cell depletion in non-human primate (NHP; i.e., rhesus macaques and cynomolgus monkeys) and human primary PBMCs. The level of B cell depletion was assessed by gating on the CD20+ cells and determining the % of dead cells via live/dead stain.

FIG. 46 shows the repeated CAR-mediated cytotoxicity of Nalm6 target cells resulting from co-culture with CD8+ T cells after being transfected with Lipid 15 LNPs comprising an anti-CD8-targeting moiety as assessed by the live cell imaging SX5 Incucyte system. Repeated addition of Nalm6 target cells to the culture was performed every 48 or 72 hours as indicated by the cell schematic beneath the x-axis.

FIG. 47 depicts the Anion Exchange Chromatography profile of VHH 15C01-GGC after capture and subsequent polish. The product was loaded on Capto Q ImpRes (Cytiva) in a 25 mM Tirs pH7.5 buffer and eluted with the same buffer with a salt gradient from 0 to 2000 mM of NaCl.

FIG. 48 depicts the Anion Exchange Chromatography profile of VHH 15C01-GGC after capture, a reduction step in TCEP and subsequent polish. The product was loaded on Capto Q ImpRes (Cytiva) in a 25 mM Tirs pH7.5 buffer and eluted with the same buffer with a salt gradient from 0 to 2000 mM of NaCl.

FIG. 49 depicts the Flow Cytometry gating strategy for ex vivo whole blood.

FIG. 50 depicts concept and compositions of an exemplary targeted LNP.

FIG. 51 depicts exemplary method for making phospholipid-PEG-ISVD conjugate.

FIG. 52 depicts an example of using click chemistry to make phospholipid-PEG-ISVD conjugate.

FIG. 53 depicts an exemplary method for making targeted LNP through antibody conjugated lipid-PEG micelles and parent LNPs.

FIGS. 54A-54G depict Lipid 15-based LNPs show highly efficient mRNA delivery to primary human T cells in vitro and 1.5% DPG-PEG shows superior transfection in vivo. FIG. 54A shows an illustration of the LNP formulation workflow. The left panel depicts how an aqueous solution containing the mRNA and an organic solution containing the various lipids are mixed by microfluidic mixing using a NanoAssemblr Ignite. The right panel depicts how a protein-based targeting moiety conjugate is post-inserted into the non-targeted LNP to form a targeted LNP (created with BioRender.com). FIG. 54B shows the fraction of DiI-positive primary human T cells, demonstrating the level of association and binding to T cells 24 hours after incubation with 1 μg/mL CD8-targeted LNPs formulated with different ionizable lipids and encapsulating mRNA encoding GFP. FIGS. 54C-54D depict the fraction of GFP-positive T cells (FIG. 54C) and the GFP MFI (FIG. 54D) evaluating the level of protein expression in T cells 24 hours after incubation with 1 μg/mL and 0.25 μg/mL CD8-targeted LNPs formulated with different ionizable lipids and encapsulating mRNA encoding GFP. Data are mean of values in n=2 replicates. The displayed p-values are from a two-way ANOVA. FIG. 54E depicts histograms of Dil and GFP values in one representative donor illustrating the differences between CD8-targeted LNPs based on Lipid 15 and ALC-0315. FIG. 54F shows an in vivo comparison of 1 mg/kg CD3-targeted (SP34) LNPs based on DLin-KC2-DMA (KC2) and with 2.5% DMG-PEG, DPG-PEG, and DSG-PEG, respectively, demonstrating the level of GFP-positive cells of CD4+ and CD8+ T cells, 24 hours after intravenous injection into humanized NSG mice. Data are from biologically independent mice (n=4 in LNP-treated groups, n=3 in vehicle group). The displayed p-values are from a two-way ANOVA. FIG. 54G shows an in vivo comparison of 0.3 mg/kg CD3-targeted (SP34) LNPs based on KC2 and with different percentages of DMG-PEG, DPG-PEG, and DPPE-PEG, demonstrating the level of GFP-positive cells of CD8+ T cells, 24 hours after intravenous injection into humanized NSG mice. Data are from biologically independent mice (n=4 in LNP groups, n=2 in vehicle group). The displayed p-values are from an ordinary one-way ANOVA.

FIGS. 55A-55E depict αCD8 ISVD-targeted LNPs show dose-dependent and specific delivery to CD8+ cells without inducing cytokine release in vitro. FIGS. 55A-55B depicts the fraction of DiI− (FIG. 55A) and GFP-positive (FIG. 55B) primary human T cells 24 hours after incubation with 0.33 μg/mL mRNA encapsulated in targeted LNPs inserted with αCD8 (anti-CD8 Fab (TRX2) or Nb8), αCD3 (anti-CD3 Fab (SP34)), or non-binding (Fab (mutOKT8)) targeting moieties in three independent donors. Each bar represents the mean of technical replicates (n=3). The displayed p-values are from a two-way ANOVA. FIG. 55C depicts the level of GFP expression over time in T cells evaluated with the Incucyte SX5 and illustrated as the Green Integrated Intensity (GCU). Data are from biologically independent replicates (n=3). FIG. 55D shows the levels of IFN-γ detected in the supernatants 24 hours after incubation with 0.33 μg/mL mRNA encapsulated in targeted LNPs. Each bar represents the mean of technical replicates (n=3). FIG. 55E provides an ex vivo whole blood experiment enabling investigation of expression in Granulocytes, B cells, T cells, and NK cells by flow cytometry (left panel). Fraction of GFP-positive cells in various cell subsets 24 hours after incubation with mRNA-encapsulating LNPs inserted with the humanized αCD8 VHH (Nb8-H3) or non-binding (Fab (mutOKT8)) targeting moieties (right panel). Data are from biologically independent replicates (n=8) (created with BioRender.com).

FIGS. 56A-56F depicts Lipid chemistry of various lipids. Chemical structures of DLin-KC2-DMA (KC2-DMA) (FIG. 56A), KC3-DMA (FIG. 56B), ALC-0315 (FIG. 56C), SM-102 (FIG. 56D), Dialkyl lipid (FIG. 56E), and phospholipid degradation (FIG. 56F).

FIGS. 57A-57C depict chemical structures of novel ionizable lipids and PEG lipids. FIG. 57A provides chemical structures of proprietary branched lipids 1, 2, 3, 4, 5, 6, 7, 8, 9, 10, 15, 16, 24A, and 26. FIG. 57B provides chemical structures of DMG-PEG, DPG-PEG, and DSG-PEG. FIG. 57C provides chemical structures of DMPE-PEG, DPPE-PEG, and DSPE-PEG.

FIGS. 58A-58B depict size and PDI of LNPs formulated with novel ionizable lipids 1-8. FIG. 58A provides diameter (DLS, nm) of LNPs based on Lipid 1 to Lipid 8 in pH 7.4 HBS, pH 6.5 MBS, post-antibody (αCD3, SP34) insertion and post-freeze-thaw (−80° C.). FIG. 58B provides polydispersity (DLS) of LNPs based on Lipid 1 to Lipid 8 in pH 7.4 HBS, pH 6.5 MBS, post-antibody (αCD3, SP34) insertion and post-freeze-thaw (−80° C.). Data are mean of values in n=2 replicates

FIGS. 59A-59B depict size and PDI of LNPs formulated with novel ionizable lipids 9, 10, 11, and 15. FIG. 59A provides diameter (DLS, nm) of LNPs based on Lipids 9, 10, 11, and 15 in pH 7.4 HBS, pH 6.5 MBS, post-antibody (αCD3, SP34) insertion and post-freeze-thaw (−80° C.). FIG. 59B provides polydispersity (DLS) of LNPs based on Lipids 9, 10, 11, and 15 in pH 7.4 HBS, pH 6.5 MBS, post-antibody (αCD3, SP34) insertion and post-freeze-thaw (−80° C.). Data are mean of values in n=2 replicates.

FIGS. 60A-60D depict LNP characterization of LNPs formulated with ionizable lipids 10, 15, 16, 24A, and 26. FIG. 60A provides diameter (DLS, nm) of targeted LNPs (αCD8, Nb8 VHH) based on Lipids 10, 15, 16, 24A, 26, and ALC-0315 pre- and post-freeze-thaw. FIG. 60B provides polydispersity of targeted LNPs (αCD8, Nb8 VHH) based on Lipids 10, 15, 16, 24A, 26, and ALC-0315 pre- and post-freeze-thaw. FIG. 60C provides zeta Potential (mV) of targeted LNPs (αCD8, Nb8 VHH) based on Lipids 10, 15, 16, 24A, 26, and ALC-0315 pre- and post-freeze-thaw in pH 5.5 MES and pH 7.4 HBS. FIG. 60D provides total mRNA recovery (ug/mL) and % dye-accessible mRNA of targeted LNPs (αCD8, Nb8 VHH) based on Lipids 10, 15, 16, 24A, 26, and ALC-0315 pre- and post-freeze-thaw.

FIGS. 61A-61E depicts transfection of human T cells with CD3-targeted LNPs based on Lipids 1, 3, and 5 pre- and post-freeze-thaw. FIG. 61A provides % GFP+ primary human T cells after transfection with CD3-targeted LNPs based on DLn-KC2-DMA (−80° C. stored), Lipid 1 (4° C. stored), Lipid 3 (4° C. stored), and Lipid 5 (4° C. stored). FIG. 61B provides % GFP+ primary human T cells after transfection with CD3-targeted LNPs based on DLn-KC2-DMA, Lipid 1, Lipid 3, and Lipid 5 after freeze-thaw cycle (−80° C. storage). FIG. 61C provides GFP MFI in primary human T cells after transfection with CD3-targeted LNPs based on DLn-KC2-DMA (−80° C. stored), Lipid 1 (4° C. stored), Lipid 3 (4° C. stored), and Lipid 5 (4° C. stored). FIG. 61D provides GFP MFI in primary human T cells after transfection with CD3-targeted LNPs based on DLn-KC2-DMA, Lipid 1, Lipid 3, and Lipid 5 after freeze-thaw cycle (−80° C. storage). FIG. 61E provides % Live primary human T cells after transfection with CD3-targeted LNPs based on DLn-KC2-DMA, Lipid 1, Lipid 3, and Lipid 5 after freeze-thaw cycle (−80° C. storage).

FIGS. 62A-62C depict transfection of human T cells with CD3-targeted LNPs based on Lipids 1 and 8 pre- and post-freeze-thaw. FIG. 62A provides % GFP+ primary human T cells after transfection with αCD3 (SP34) targeted LNPs based on DLn-KC2-DMA, Lipid 1 (4° C. stored), Lipid 8 (4° C. stored), and Lipid 8 (−80° C. stored). FIG. 62B provides GFP MFI in primary human T cells after transfection with αCD3 (SP34) targeted LNPs based on DLn-KC2-DMA, Lipid 1 (4° C. stored), Lipid 8 (4° C. stored), and Lipid 8 (−80° C. stored). FIG. 62C provides % Live primary human T cells after transfection with αCD3 (SP34) targeted LNPs based on DLn-KC2-DMA, Lipid 1 (4° C. stored), Lipid 8 (4° C. stored), and Lipid 8 (−80° C. stored).

FIGS. 63A-63D depict transfection of human T cells with CD3-targeted LNPs based on Lipids 8, 9, and 10 pre- and post-freeze-thaw. FIG. 63A provides % GFP+ primary human T cells after transfection with αCD3 (SP34) targeted LNPs based on DLn-KC2-DMA (−80° C. stored), Lipid 8 (4° C. stored), Lipid 9 (4° C. stored), and Lipid 10 (4° C. stored). FIG. 63B provides % GFP+ primary human T cells after transfection with αCD3 (SP34) targeted LNPs based on DLn-KC2-DMA (−80° C. stored), Lipid 8 (−80° C. stored), and Lipid 10 (−80° C. stored). FIG. 63C provides GFP MFI in primary human T cells after transfection with αCD3 (SP34) targeted LNPs based on DLn-KC2-DMA (−80° C. stored), Lipid 8 (4° C. stored), Lipid 9 (4° C. stored), and Lipid 10 (4° C. stored). FIG. 63D provides GFP MFI in primary human T cells after transfection with αCD3 (SP34) targeted LNPs based on DLn-KC2-DMA (−80° C. stored), Lipid 8 (−80° C. stored), and Lipid 10 (−80° C. stored).

FIGS. 64A-64E depict transfection of human T cells with CD3-targeted LNPs based on Lipids 3, 4, 9, and 15 pre- and post-freeze-thaw. FIG. 64A provides % GFP+ primary human T cells after transfection with αCD3 (SP34) targeted LNPs based on DLn-KC2-DMA (−80° C. stored), Lipid 3 (4° C. stored), Lipid 4 (4° C. stored), Lipid 9 (4° C. stored), and Lipid 15 (4° C. stored). FIG. 64B provides % GFP+ primary human T cells after transfection with αCD3 (SP34) targeted LNPs based on DLn-KC2-DMA (−80° C. stored), Lipid 3 (−80° C. stored), Lipid 4 (−80° C. stored), Lipid 9 (−80° C. stored), and Lipid 15 (−80° C. stored). FIG. 64C provides GFP MFI in primary human T cells after transfection with αCD3 (SP34) targeted LNPs based on DLn-KC2-DMA (−80° C. stored), Lipid 3 (4° C. stored), Lipid 4 (4° C. stored), Lipid 9 (4° C. stored), and Lipid 15 (4° C. stored). FIG. 64D provides GFP MFI in primary human T cells after transfection with αCD3 (SP34) targeted LNPs based on DLn-KC2-DMA (−80° C. stored), Lipid 3 (−80° C. stored), Lipid 4 (−80° C. stored), Lipid 9 (−80° C. stored), and Lipid 15 (−80° C. stored). FIG. 64E provides % Live primary human T cells after transfection with αCD3 (SP34) targeted LNPs based on DLn-KC2-DMA (−80° C. stored), Lipid 3 (−80° C. stored), Lipid 4 (−80° C. stored), Lipid 9 (−80° C. stored), and Lipid 15 (−80° C. stored).

FIGS. 65A-65E depict transfection of human T cells with CD8-targeted vs non-targeted LNPs based on Lipids 3, 4, 9, and 15. FIG. 65A provides % GFP+ primary human T cells after transfection with αCD8 (Nb8) targeted or non-targeted LNPs based on Lipid 3, Lipid, 4, Lipid 9, and Lipid 15. FIG. 65B provides GFP MFI in primary human T cells after transfection with αCD8 (Nb8) targeted or non-targeted LNPs based on Lipid 3, Lipid, 4, Lipid 9, and Lipid 15. FIG. 65C provides % DiI+ primary human T cells after transfection with αCD8 (Nb8) targeted or non-targeted LNPs based on Lipid 3, Lipid, 4, Lipid 9, and Lipid 15. FIG. 65D provides DiI MFI in primary human T cells after transfection with αCD8 (Nb8) targeted or non-targeted LNPs based on Lipid 3, Lipid, 4, Lipid 9, and Lipid 15. FIG. 65E provides % Live primary human T cells after transfection with αCD8 (Nb8) targeted or non-targeted LNPs based on Lipid 3, Lipid, 4, Lipid 9, and Lipid 15.

FIGS. 66A-66E depict transfection of human T cells with CD8-targeted LNPs based on Lipids 10, 15, 16, 24A, and 26 pre- and post-freeze-thaw. FIG. 66A provides % GFP+primary human T cells after transfection with αCD8 (Nb8) targeted LNPs based on Lipids 10, 15, 16, 24A, and 26 and comparator lipid ALC-0315 pre- and post-freeze-thaw. FIG. 66B provides GFP MFI in primary human T cells after transfection with αCD8 (Nb8) targeted LNPs based on Lipids 10, 15, 16, 24A, and 26 and comparator lipid ALC-0315 pre- and post-freeze-thaw. FIG. 66C provides % DiI+primary human T cells after transfection with αCD8 (Nb8) targeted LNPs based on Lipids 10, 15, 16, 24A, and 26 and comparator lipid ALC-0315 pre- and post-freeze-thaw. FIG. 66D provides DiI MFI in primary human T cells after transfection with αCD8 (Nb8) targeted LNPs based on Lipids 10, 15, 16, 24A, and 26 and comparator lipid ALC-0315 pre- and post-freeze-thaw. FIG. 66E provides % Live primary human T cells after transfection with αCD8 (Nb8) targeted LNPs based on Lipids 10, 15, 16, 24A, and 26 and comparator lipid ALC-0315 pre- and post-freeze-thaw.

FIGS. 67A-67F depict αCD3-targeted LNPs induce cytokine secretion and upregulation of CD69 in human T cells in vitro. FIG. 67A provides the Dil MFI and FIG. 67B provides GFP MFI of human T cells 24 hours after incubation with varying doses of mRNA-encapsulating LNPs inserted with αCD8 (Nb8 or CD8 Fab), αCD3 (CD3 Fab), or non-binding (Fab control) targeting moieties in 3 independent human donors. Data are from technical replicates (n=3). FIG. 67C provides CD69 MFI of human T cells 24 hours after incubation with varying doses of mRNA-encapsulating LNPs inserted with αCD8 (Nb8 or CD8 Fab), αCD3 (CD3 Fab), or non-binding (Fab control) targeting moieties in 3 independent human donors. Data are from technical replicates (n=3). FIG. 67D provides histograms of CD69 values in one representative donor (Donor 1) illustrating the differences between cells treated with αCD3-targeted LNPs and αCD8-targeted LNPs. FIG. 67E provides the level of DiI-LNP association over time with T cells evaluated with the Incucyte SX5 and illustrated as the Near InfraRed (NIR) Integrated Intensity (NIRCU). Data are from biologically independent replicates (n=3). FIG. 67F provides levels of TNF-α detected in the supernatants 24 hours after incubation with varying doses of mRNA-encapsulating LNPs in 3 independent human donors. Data are from technical replicates (n=3). The displayed p-values are from a two-way ANOVA.

FIGS. 68A-68E depict comparison of LNPs inserted with humanized variants of Nb8. FIG. 68A provides the fraction of live T cells 24 hours after incubation with 1, 0.3, and 0.06 μg/mL mRNA encapsulated in targeted LNPs inserted with humanized variants of Nb8 at different densities. Each bar represents the mean of technical duplicates (n=2). FIG. 68B provides the DiI-positive fraction and FIG. 68C provides DiI MFI of primary human T cells 24 hours after incubation with 1, 0.3, and 0.06 μg/mL mRNA encapsulated in targeted LNPs inserted with humanized variants of Nb8 at different densities. Data are mean of values in n=2 replicates. FIG. 68D provides the GFP-positive fraction and FIG. 68E provides GFP MFI of primary human T cells 24 hours after incubation with 1, 0.3, and 0.06 μg/mL mRNA encapsulated in targeted LNPs inserted with humanized variants of Nb8 at different densities. Data are mean of values in n=2 replicates.

FIGS. 69A-69B depict the comparison of CAR expression level on NK cells and CD8+ T cells (FIG. 69A) and B cell aplasia (FIG. 69B) in the tissues (e.g., spleen) of rhesus macaques dosed with CD8-targeted LNP comprising mRNA encoding anti-CD19 CAR #1. FIG. 69A shows the percent (%) of anti-CD19 CAR #1 expression on CD8+ T cells (top panel) and NK cells (bottom panel). FIG. 69B shows the B cell count (top panel) and percent (%) of live B cells in the spleens of rhesus macaques dosed with CD8-targeted LNP comprising mRNA encoding anti-CD19 CAR #1.

FIGS. 70A-70B depict the degree of B cell depletion in spleen and axillary lymph nodes of rhesus macaques dosed with CD8-targeted LNP comprising mRNA encoding anti-CD19 CAR #1 or anti-CD19 CAR #3. FIG. 70A shows the results of immunohistochemical (IHC) staining of the spleens rhesus macaques dosed with CD8-targeted LNP comprising mRNA encoding anti-CD19 CAR #1 or anti-CD19 CAR #3 for CD19-positive cells. FIG. 70B showsshows the results of immunohistochemical (IHC) staining of the spleens of rhesus macaques dosed with CD8-targeted LNP comprising mRNA encoding anti-CD19 CAR #1 or anti-CD19 CAR #3 for CD20-positive cells.

### DETAILED DESCRIPTION—LIPID NANOPARTICLES

The following description sets forth exemplary methods, parameters and the like. It should be recognized, however, that such description is not intended as a limitation on the scope of the present disclosure but is instead provided as a description of exemplary embodiments.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts. In addition, when a chemical group is a diradical, for example, it is understood a that the chemical groups can be bonded to their adjacent atoms in the remainder of the structure in one or both orientations, for example, —OC(O)— is interchangeable with —C(O)O— or —OC(S)— is interchangeable with —C(S)O—.

The terms “a” and “an” as used herein mean “one or more” and include the plural unless the context is inappropriate. In some embodiments, “one or more” is 1 or 2. In some embodiments, “one or more” is 1, 2, or 3. In some embodiments, “one or more” is 1, 2, 3, or 4. In some embodiments, “one or more” is 1, 2, 3, 4, or 5. In some embodiments, “one or more” is 1, 2, 3, 4, 5, or more.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, or C₁-C₆alkyl, respectively. In some embodiments, alkyl is optionally substituted. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. In some embodiments, alkylene is optionally substituted. An exemplary alkylene group is —CH2CH2-.

The term “haloalkyl” refers to an alkyl group that is substituted with at least one halogen. For example, —CH₂F, —CHF₂, —CF₃, —CH₂CF₃, —CF₂CF₃, and the like.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

The term “oxo” is art-recognized and refers to a “—O” substituent. For example, a cyclopentane substituted with an oxo group is cyclopentanone.

The term “morpholinyl” refers to a substituent having the structure of:

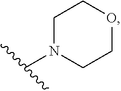 [SMILES: *N1CCOCC1]

which is optionally substituted.

The term “piperidinyl” refers to a substituent having a structure of:

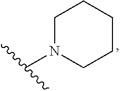 [SMILES: *N1CCCCC1]

which is optionally substituted.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, “optionally substituted” is equivalent to “unsubstituted or substituted.” In some embodiments, “optionally substituted” indicates that the designated atom or group is optionally substituted with one or more substituents independently selected from optional substituents provided herein. In some embodiments, optional substituent may be selected from the group consisting of: C₁₋₆alkyl, cyano, halogen, —O—C₁₋₆alkyl, C₁₋₆haloalkyl, C₃₋₇cycloalkyl, 3- to 7-membered heterocyclyl, 5- to 6-membered heteroaryl, and phenyl. In some embodiments, optional substituent is alkyl, cyano, halogen, halo, azide, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl, or heteroaryl. In some embodiments, optional substituent is —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13}, or —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

The term “haloalkyl” refers to an alkyl group that is substituted with at least one halogen. For example, —CH₂F, —CHF₂, —CF₃, —CH₂CF₃, —CF₂CF₃, and the like.

The term “cycloalkyl” refers to a monovalent saturated cyclic, bicyclic, bridged cyclic (e.g., adamantyl), or spirocyclic hydrocarbon group of 3-12, 3-10, 3-8, 4-8, or 4-6 carbons, referred to herein, e.g., as “C₄₋₈cycloalkyl,” derived from a cycloalkane. In some embodiments, cycloalkyl is optionally substituted. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. In some embodiments, heterocyclyl is optionally substituted. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems (e.g., fused, spiro, bridged bicyclic). A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “aryl” is art-recognized and refers to a carbocyclic aromatic group. In some embodiments, aryl is optionally substituted. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6- to 10-membered ring structure. In some embodiments, the aryl group is a C₆-C₁₄ aryl.

The term “heteroaryl” is art-recognized and refers to aromatic groups that include at least one ring heteroatom. In some embodiments, heteroaryl is optionally substituted. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula —N(R¹⁰)(R¹¹), wherein R¹⁰ and R¹¹ each independently represent hydrogen, alkyl, cycloalkyl, heterocyclyl, alkenyl, aryl, aralkyl, or (CH₂)ₘ—R¹²; or R¹⁰ and R¹¹, taken together with the N atom to which they are attached complete a heterocycle having from 4 to 8 atoms in the ring structure; R 12 represents an aryl, a cycloalkyl, a cycloalkenyl, a heterocycle or a polycycle; and m is zero or an integer in the range of 1 to 8. In certain embodiments, R¹⁰ and R¹¹ each independently represent hydrogen, alkyl, alkenyl, or —(CH₂)ₘ—R¹².

The terms “alkoxyl” or “alkoxy” are art-recognized and refer to an alkyl group, as defined above, having an oxygen radical attached thereto. In some embodiments, alkoxyl is optionally substituted. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, O-alkynyl, —O—(CH₂)ₘ—R¹², where m and R¹² are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol “=” denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The present disclosure also embraces isotopically labeled compounds of the present disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in, e.g., the Examples herein by substituting an isotopically labeled reagent for a non-isotopically labeled reagent.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an affinity ligand, in particular, an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable excipient” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Remington's The Science and Practice of Pharmacy, 21st Edition, A. R. Gennaro; Lippincott, Williams & Wilkins, Baltimore, MD, 2006.

As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the present disclosure and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

Abbreviations as used herein include diisopropylethylamine (DIPEA); 4-dimethylaminopyridine (DMAP); tetrabutylammonium iodide (TBAI); 1-ethyl-3-(3-dimethylaminopropyl) carbodiimide (EDC); benzotriazol-1-yl-oxytripyrrolidinophosphonium hexafluorophosphate (PyBOP), 9-Fluorenylmethoxycarbonyl (Fmoc), tetrabutyldimethylsilyl chloride (TBDMSCl), hydrogen fluoride (HF), phenyl (Ph), bis(trimethylsilyl)amine (HMDS), dimethylformamide (DMF); methylene chloride (DCM); tetrahydrofuran (THF); high-performance liquid chromatography (HPLC); mass spectrometry (MS), evaporative light scattering detector (ELSD), electrospray(ES)); nuclear magnetic resonance spectroscopy (NMR).

As used herein, the term “effective amount” refers to the amount of a compound (e.g., a nucleic acid, e.g., an mRNA) sufficient to effect beneficial or desired results. An effective amount can be administered in one or more administrations, applications or dosages and is not intended to be limited to a particular formulation or administration route. The term effective amount can be considered to include therapeutically and/or prophylactically effective amounts of a compound.

The phrase “therapeutically effective amount” as used herein means that amount of a compound (e.g., a nucleic acid, e.g., an mRNA), material, or composition comprising a compound (e.g., a nucleic acid, e.g., an mRNA) which is effective for producing some desired therapeutic effect in at least a sub-population of cells in a mammal, for example, a human, or a subject (e.g., a human subject) at a reasonable benefit/risk ratio applicable to any medical treatment.

The phrase “prophylactically effective amount” as used herein means that amount of a compound (e.g., a nucleic acid, e.g., an mRNA), material, or composition comprising a compound (e.g., a nucleic acid, e.g., an mRNA) which is effective for producing some desired prophylactic effect in at least a sub-population of cells in a mammal, for example, a human, or a subject (e.g., a human subject) by reducing, minimizing or eliminating the risk of developing a condition or the reducing or minimizing severity of a condition at a reasonable benefit/risk ratio applicable to any medical treatment.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As used herein, unless otherwise indicated, the term “antibody” means any antigen-binding molecule or molecular complex comprising at least one complementarity determining region (CDR) that specifically binds to or interacts with a particular antigen. It is understood the term encompasses an intact antibody (e.g., an intact monoclonal antibody), or a fragment thereof, such as an Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified or engineered. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). The term also encompasses an immunoglobulin single variable domain, such as a VHH (including a humanized VHH), a VH (including a camelized VH, a human VH, a camelized human VH, and a dAb) or a VL.

As used here, an “antibody that binds to X” (i.e., X being a particular antigen), or “an anti-X antibody”, is an antibody that specifically recognizes the antigen X.

As used herein, a “buried interchain disulfide bond” or an “interchain buried disulfide bond” refers to a disulfide bond on a polypeptide which is not readily accessible to water soluble reducing agents, or is effectively “buried” in the hydrophobic regions of the polypeptide, such that it is unavailable to both reducing agents and for conjugation to other hydrophilic PEGs. Buried interchain disulfide bonds are further described in WO2017096361A1, which is incorporated by reference in its entirety.

As used herein, specificity of the targeted delivery by an LNP is defined by the ratio between % of a desired immune cell type that receives the delivered nucleic acid (e.g., on-target delivery), and % of an undesired immune cell type that is not meant to be the destination of the delivery, but receives the delivered nucleic acid (e.g., off-target delivery). For example, the specificity is higher when more desired immune cells receive the delivered nucleic acid, while less undesired immune cells receive the delivered nucleic acid. Specificity of the targeted delivery by an LNP can also be defined by the ratio of amount of nucleic acid being delivered to the desired immune cells (e.g., on-target delivery) and amount of nucleic acid being delivered to the undesired immune cells (e.g., off-target delivery). Specificity of the delivery can be determined using any suitable method. As a non-limiting example, expression level of the nucleic acid in the desired immune cell type can be measured and compared to that of a different immune cell type that is not meant to be the destination of the delivery.

As used herein, in some embodiments, a reference LNP is an LNP that does not have the immune cell targeting group but is otherwise the same as the tested LNP. In some other embodiments, a reference LNP is an LNP that has a different ionizable cationic lipid but is otherwise the same as the tested LNP. In some embodiments, a reference LNP comprises D-Lin-MC3-DMA as the ionizable cationic lipid which is different from the ionizable cationic lipid in a tested LNP, but is otherwise the same as the tested LNP.

As used herein, a humanized antibody is an antibody which is wholly or partially of non-human origin and whose protein sequence has been modified to replace certain amino acids, for instance that occur at the corresponding position(s) in the framework regions of the VH and VL domains in a sequence of antibody from a human being, to increase its similarity to antibodies produced naturally in humans, in order to avoid or minimize an immune response in humans. For example, using techniques of genetic engineering, the variable domains of a non-human antibodies of interest may be combined with the constant domains of human antibodies. The constant domains of a humanized antibody are most of the time human CH and CL domains.

As used herein, the term “structural lipid” refers to sterols and also to lipids containing sterol moieties.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description includes each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the present disclosure unless claimed. No language in the specification should be construed as indicating any non-claimed element as essential to the practice of the present invention.

As used herein, the term “pseudouridine” refers to the natural product which is a C-glycosyl pyrimidine that consists of uracil having a beta-D-ribofuranosyl residue attached at position 5 (i.e., 5-(beta-D-Ribofuranosyl) uracil). In some embodiments, the term refers to m′acphi/(1-methyl-3-(3-amino-3-carboxypropyl) pseudouridine. In another embodiment, the term refers to mlvP (1-methylpseudouridine). In another embodiment, the term refers to *|/m (2′-O-methylpseudouridine. In another embodiment, the term refers to m5D (5-methyldihydrouridine). In another embodiment, the term refers to m3\|/(3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

As used herein, the terms “lipid-PEG” and “PEG-lipid” are interchangeable, referring to PEG derivatives in which PEG is attached with a lipid moiety. PEG-lipid can be used to improve circulation times for liposome encapsulated (LNP) drugs and reduce non-specific uptakes. If the lipid is a phospholipid, the molecule can be referred as “phospholipid-PEG” or “PEG-phospholipid”. Any suitable chemistry may be used to conjugate a polypeptide to the PEG of the PEG-lipid, see Parhiz et al., Journal of Controlled Release 291:106-115, 2018; Kolb et al., Angewandte Chemie International Edition 40(11):2004-2021, 2001; and Evans, Australian Journal of Chemistry 60(6):384-395, 2007. For example, lipid-PEG-maleimide, lipid-PEG-cysteine, lipid-PEG-alkyne, PEG-dibenzocyclooctyne (DBCO), lipid-PEG-bromo maleimide, lipid-PEG-alkylnoic amide, PEG-alkynoic imide, and lipid-PEG-azide can be used to produce a Lipd-PEG-polypeptide conjugate.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In some embodiments, when a domain, antibody, or sequence is derived from another domain, antibody, or sequence, then the domain, antibody, or sequence is the same as the other domain, antibody, or sequence. In some embodiments, when a domain, antibody, or sequence is derived from another domain, antibody, or sequence, the domain, antibody, or sequence has at least 80%, at least 85%, at least 90%, at least 95%, at least 96%, at least 97%, at least 98%, or at least 99% identity with the other domain, antibody, or sequence. In some embodiments, when a domain, antibody, or sequence is derived from another domain, antibody, or sequence, the domain, antibody, or sequence has 3, 2, or 1 amino acid difference.

### II. Antibody Moieties (Immunoglobulin Single Variable Domain)

The present disclosure provides immune cell targeting LNPs comprising an immune cell targeting group. In some embodiments, the immune cell targeting group of the LNPs as described herein comprises an antibody moiety, wherein the antibody moiety is selected from the group consisting of: a full-length antibody, an scFv, a VH, a VL, an scFv-scFv, an Fv, a Fab, a Fab′, a (Fab′)₂, a minibody, a diabody, a domain antibody variant (dAb), a single domain antibody (sdAb or VHH), a camelid VHH antibody, a Nanobody® ISVD, a fibronectin 3 domain variant, an ankyrin repeat variant, and other antigen-specific binding domains derived from other protein scaffolds. In some embodiments, the immune cell targeting group of the LNPs as described herein comprises an immunoglobulin single variable domain (ISVD), such as a VHH (including a humanized VHH), a VH (including a camelized VH, a human VH, a camelized human VH, and a dAb) or a VL.

The term “immunoglobulin single variable domain” (ISVD), interchangeably used with “single variable domain,” defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both VH and VL will contribute to the antigen binding site, i.e., a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain. Hence, the antigen binding site of an immunoglobulin single variable domain is formed by no more than three CDRs.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a V_{H}, V_{HH}, including a camelized V_{H} or humanized V_{HH}. In one embodiment, it is a V_{HH}, including a camelized V_{H} or humanized V_{HH}. Heavy chain ISVDs can be derived from a conventional four-chain antibody or from a heavy chain antibody.

For example, the immunoglobulin single variable domain may be a (single) domain antibody (or an amino acid sequence that is suitable for use as a single domain antibody), a “dAb” or dAb (or an amino acid sequence that is suitable for use as a dAb) or a Nanobody® ISVD (as defined herein and including but not limited to a V_{HH}); other single variable domains, or any suitable fragment of any one thereof.

In particular, the immunoglobulin single variable domain may be a Nanobody® ISVD (such as a V_{HH}, including a humanized V_{HH} or camelized V_{H}) or a suitable fragment thereof. Nanobody® and Nanobodies® is a registered trademark of Ablynx N.V.

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. 1993 (Nature 363:446-448). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans 2001 (Reviews in Molecular Biotechnology 74:277-302).

For the term “dAb's” and “domain antibody”, reference is for example made to Ward et al. 1989 (Nature 341:544), to Holt et al. 2003 (Trends Biotechnol. 21:484); as well as to for example WO 2004/068820, WO 2006/030220, WO 2006/003388 and other published patent applications of Domantis Ltd. It should also be noted that, although less preferred in the context of the present invention because they are not of mammalian origin, single variable domains can be derived from certain species of shark (for example, the so-called “IgNAR domains”, see for example WO 2005/18629).

Typically, the generation of immunoglobulins involves the immunization of experimental animals, fusion of immunoglobulin producing cells to create hybridomas and screening for the desired specificities. Alternatively, immunoglobulins can be generated by screening of naïve, immune or synthetic libraries, e.g., by phage display.

The generation of immunoglobulin sequences, such as VHHs, has been described extensively in various publications, among which WO 1994/04678, Hamers-Casterman et al. 1993 (Nature 363:446-448) and Muyldermans et al. 2001 (Reviews in Molecular Biotechnology 74:277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be used herein. Also, fully human, humanized or chimeric sequences can be used in the method described herein. For example, camelid immunoglobulin sequences and humanized camelid immunoglobulin sequences, or camelized domain antibodies, e.g., camelized dAb as described by Ward et al. 1989 (Nature 341:544), WO 1994/04678, and Davis and Riechmann (1994, Febs Lett., 339:285-290; and 1996, Prot. Eng., 9:531-537) can be used herein. Moreover, the ISVDs are fused forming a multivalent and/or multispecific construct (for multivalent and multispecific polypeptides containing one or more V_{HH} domains and their preparation, reference is also made to Conrath et al. 2001 (J. Biol. Chem., Vol. 276, 10. 7346-7350) as well as to for example WO 1996/34103 and WO 1999/23221).

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the prior art (e.g., WO 2008/020079). Again, it should be noted that such humanized V_{HH}S can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the description in the prior art (e.g., Davies and Riechman 1994, FEBS 339:285; 1995, Biotechnol. 13:475; 1996, Prot. Eng. 9:531; and Riechman 1999, J. Immunol. Methods 231:25). Such “camelizing” substitutions are inserted at amino acid positions that form and/or are present at the V_{H}-V_{L} interface, and/or at the so-called Camelidae hallmark residues, as defined herein (see for example WO 1994/04678 and Davies and Riechmann (1994 and 1996, supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

The amino acid residues of an ISVD can be numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 2000 (J. Immunol. Methods 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering. That is, one or more positions according to the Kabat numbering may not be occupied in the actual sequence, or the actual sequence may contain more amino acid residues than the number allowed for by the Kabat numbering. This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, unless indicated otherwise, CDR sequences were determined according to the AbM numbering as described in Kontermann and Dübel (Eds. 2010, Antibody Engineering, vol 2, Springer Verlag Heidelberg Berlin, Martin, Chapter 3, pp. 33-51). According to this method, FR1 of an ISVD comprises the amino acid residues at positions 1-25, CDR1 of an ISVD comprises the amino acid residues at positions 26-35, FR2 of an ISVD comprises the amino acids at positions 36-49, CDR2 of an ISVD comprises the amino acid residues at positions 50-58, FR3 of an ISVD comprises the amino acid residues at positions 59-94, CDR3 of an ISVD comprises the amino acid residues at positions 95-102, and FR4 of an ISVD comprises the amino acid residues at positions 103-113.

Determination of CDR regions may also be done according to different methods. In the CDR determination according to Kabat, FR1 of an ISVD comprises the amino acid residues at positions 1-30, CDR1 of an ISVD comprises the amino acid residues at positions 31-35, FR2 of an ISVD comprises the amino acids at positions 36-49, CDR2 of an ISVD comprises the amino acid residues at positions 50-65, FR3 of an ISVD comprises the amino acid residues at positions 66-94, CDR3 of an ISVD comprises the amino acid residues at positions 95-102, and FR4 of an ISVD comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a V_{L}-sequence) and/or from a heavy chain variable domain (e.g., a V_{H}-sequence or V_{HH} sequence). In one particular aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence described herein may contain one or more of hallmark residues (as defined herein), such that the ISVD sequence is a Nanobody® ISVD, such as, e.g., a V_{HH}, including a humanized V_{HH} or camelized V_{H}. Non-limiting examples of (suitable combinations of) such framework sequences will become clear from the further disclosure herein.

The total number of amino acid residues in a V_{H} domain and a V_{HH} domain will usually be in the range of from 110 to 120, often between 112 and 115. It should however be noted that smaller and longer sequences may also be suitable for the purposes described herein.

However, it should be noted that the ISVDs described herein is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVDs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, Nanobody® ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a Nanobody® ISVD can be defined as an immunoglobulin sequence with the (general) structure FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISVD can be an immunoglobulin sequence with the (general) structure FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody® ISVD can be an immunoglobulin sequence with the (general) structure FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table A below.

TABLE A
Hallmark Residues in Nanobody ® ISVDs

| Position | Human VH3 | Hallmark Residues |
|---|---|---|
| 11 | L, V; predominantly L | L, S, V, M, W, F, T, Q, E, A, R, G, K, Y, N, P, I; |
| | | preferably L |
| 37 | V, I, F; usually V | F⁽¹⁾, Y, V, L, A, H, S, I, W, C, N, G, D, T, P, |
| | | preferably F⁽¹⁾ or Y |
| 44⁽⁸⁾ | G | E⁽³⁾, Q⁽³⁾, G⁽²⁾, D, A, K, R, L, P, S, V, H, T, N, W, |
| | | M, I; |
| | | preferably G⁽²⁾, E⁽³⁾ or Q⁽³⁾; most preferably G⁽²⁾ or |
| | | Q⁽³⁾ |
| 45⁽⁸) | L | L⁽²⁾, R⁽³⁾, P, H, F, G, Q, S, E, T, Y, C, I, D, V; |
| | | preferably L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, |
| | | K, Q, N, D; preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | P⁽⁵⁾, S, H, L, A, V, I, T, F, D, R, Y, N, Q, G, E; |
| | | preferably P |
| 103 | W | W⁽⁴⁾, R⁽⁶⁾, G, S, K, A, M, Y, L, F, T, N, V, Q, P⁽⁶⁾, E, |
| | | C; preferably W |
| 104 | G | G, A, S, T, D, P, N, E, C, L; preferably G |
| 108 | L, M or T; predominantly | Q, L⁽⁷⁾, R, P, E, K, S, T, M, A, H; preferably Q or |
| | L | L⁽⁷⁾ |

Notes:
⁽¹⁾In particular, but not exclusively, in combination with KERE (SEQ ID NO: 372) or KQRE (SEQ ID NO: 373) at positions 43-46.
⁽²⁾Usually as GLEW (SEQ ID NO: 374) at positions 44-47.
⁽³⁾Usually as KERE (SEQ ID NO: 372) or KQRE (SEQ ID NO: 373) at positions 43-46, e.g., as KEREL (SEQ ID NO: 375), KEREF (SEQ ID NO: 376), KQREL (SEQ ID NO: 377), KQREF (SEQ ID NO: 378), KEREG (SEQ ID NO: 379), KQREW (SEQ ID NO: 380) or KQREG (SEQ ID NO: 381) at positions 43-47. Alternatively, also sequences such as TERE (SEQ ID NO: 382) (for example TEREL (SEQ ID NO: 383)), TQRE (SEQ ID NO: 384) (for example TQREL (SEQ ID NO: 385)), KECE (SEQ ID NO: 386) (for example KECEL (SEQ ID NO: 387) or KECER (SEQ ID NO: 388)), KQCE (SEQ ID NO: 389) (for example KQCEL (SEQ ID NO: 390)), RERE (SEQ ID NO: 391) (for example REREG (SEQ ID NO: 392)), RQRE (SEQ ID NO: 393) (for example RQREL (SEQ ID NO: 394), RQREF (SEQ ID NO: 395) or RQREW (SEQ ID NO: 396)), QERE (SEQ ID NO: 397) (for example QEREG (SEQ ID NO: 398)), QQRE (SEQ ID NO: 399), (for example QQREW (SEQ ID NO: 400), QQREL (SEQ ID NO: 401) or QQREF (SEQ ID NO: 402)), KGRE (SEQ ID NO: 403) (for example KGREG (SEQ ID NO: 404)), KDRE (SEQ ID NO: 405) (for example KDREV (SEQ ID NO: 406)) are possible. Some other possible, but less preferred sequences include for example DECKL (SEQ ID NO: 407) and NVCEL (SEQ ID NO: 408).
⁽⁴⁾With both GLEW (SEQ ID NO: 374) at positions 44-47 and KERE (SEQ ID NO: 372) or KQRE (SEQ ID NO: 373) at positions 43-46.
⁽⁵⁾Often as KP or EP at positions 83-84 of naturally occurring VHH domains.
⁽⁶⁾In particular, but not exclusively, in combination with GLEW (SEQ ID NO: 374) at positions 44-47.
⁽⁷⁾With the proviso that when positions 44-47 are GLEW (SEQ ID NO: 374), position 108 is always Q in (non-humanized) VHH sequences that also contain a W at 103.
⁽⁸⁾The GLEW group also contains GLEW-like sequences at positions 44-47, such as for example GVEW (SEQ ID NO: 409), EPEW (SEQ ID NO: 410), GLER (SEQ ID NO: 411), DQEW (SEQ ID NO: 412), DLEW (SEQ ID NO: 413), GIEW (SEQ ID NO: 414), ELEW (SEQ ID NO: 415), GPEW (SEQ ID NO: 416), EWLP (SEQ ID NO: 417), GPER (SEQ ID NO: 418), GLER (SEQ ID NO: 411) and ELEW (SEQ ID NO: 415).

In one embodiment, the immunoglobulin single variable domain has certain amino acid substitutions in the framework regions effective in preventing or reducing binding by so-called “pre-existing antibodies” to the polypeptides. To this end, in one embodiment, the polypeptide comprises a valine (V) at amino acid position 11 and a leucine (L) at amino acid position 89 (according to Kabat numbering) in at least one ISVD. In one embodiment, the polypeptide comprises a valine (V) at amino acid position 11 and a leucine (L) at amino acid position 89 (according to Kabat numbering) in each ISVD. Accordingly, part of the present disclosure are also ISVDs and polypeptides as described above that have been sequence optimized with a valine (V) at amino acid position 11 and a leucine (L) at amino acid position 89 (according to Kabat numbering) in at least one ISVD, such as in all ISVDs. Examples of ISVDs and polypeptides that comprises a valine (V) at amino acid position 11 and a leucine (L) at amino acid position 89 (according to Kabat numbering) in at least one ISVD are depicted in Table C-2 (SEQ ID NOs: 161-169, 171-179 and 28-36 and 44).

In one embodiment, the ISVD or polypeptide has a C-terminal end of the sequence VTVSS(X)n (SEQ ID NO: 353), in which n is 1 to 10, preferably 1 to 5, such as 1, 2, 3, 4 or 5, and in which each X is an amino acid residue that is independently chosen. In one embodiment, the polypeptide comprises such an ISVD at its C-terminal end. In one embodiment, n is 1 or 2, such as 1. In one embodiment, X is a naturally occurring amino acid. In one embodiment, X is chosen from the group consisting of alanine (A), glycine (G), valine (V), leucine (L) or isoleucine (I).

In another embodiment the polypeptide comprises a lysine (K) or glutamine (Q) at position 110 (according to Kabat numbering) in at least one ISVD. In another embodiment, the ISVD comprises a lysine (K) or glutamine (Q) at position 112 (according to Kabat numbering) in at least one ISVD. In these embodiments, the C-terminus of the ISVD is VKVSS (SEQ ID NO: 354), VQVSS (SEQ ID NO: 355), VTVKS (SEQ ID NO: 356), VTVQS (SEQ ID NO: 357), VKVKS (SEQ ID NO: 358), VKVQS (SEQ ID NO: 359), VQVKS (SEQ ID NO: 360), or VQVQS (SEQ ID NO: 361) such that after addition of a single alanine the C-terminus of the polypeptide for example comprises the sequence VTVSSA (SEQ ID NO: 362), VKVSSA (SEQ ID NO: 363), VQVSSA (SEQ ID NO: 364), VTVKSA (SEQ ID NO: 365), VTVQSA (SEQ ID NO: 366), VKVKSA (SEQ ID NO: 367), VKVQSA (SEQ ID NO: 368), VQVKSA (SEQ ID NO: 369), or VQVQSA (SEQ ID NO: 370). In one embodiment, the polypeptide comprises a valine (V) at amino acid position 11 and a leucine (L) at amino acid position 89 (according to Kabat numbering) in each ISVD, optionally a lysine (K) or glutamine (Q) at position 110 (according to Kabat numbering) in at least one ISVD, and comprises an extension of 1 to 5 (naturally occurring) amino acids (as defined above), such as a single alanine (A) extension, at the C-terminus of the C-terminal ISVD, such that the C-terminus of the polypeptide for example comprises the sequence VTVSSA (SEQ ID NO: 362), VKVSSA (SEQ ID NO: 363) or VQVSSA (SEQ ID NO: 364). See e.g. WO2012/175741 and WO2015/173325 for further information in this regard.

The immunoglobulin single variable domains may form part of a protein or polypeptide, which may comprise or essentially consist of one or more (at least one) immunoglobulin single variable domains and which may optionally further comprise one or more further amino acid sequences (all optionally linked via one or more suitable linkers). The term “immunoglobulin single variable domain” may also encompass such polypeptides. The one or more immunoglobulin single variable domains may be used as a binding unit in such a protein or polypeptide, which may optionally contain one or more further amino acids that can serve as a binding unit, so as to provide a monovalent, multivalent or multispecific polypeptide of the present disclosure, respectively (for multivalent and multispecific polypeptides containing one or more VHH domains and their preparation, reference is also made to Conrath et al. 2001 (J. Biol. Chem. 276:7346), as well as to for example WO 1996/34103, WO 1999/23221 and WO 2010/115998).

The polypeptides may comprise or essentially consist of one immunoglobulin single variable domain, as outlined above. Such polypeptides are also referred to herein as monovalent polypeptides.

The term “multivalent” indicates the presence of multiple ISVDs in a polypeptide. In one embodiment, the polypeptide is “bivalent”, i.e., comprises or consists of two ISVDs. In one embodiment, the polypeptide is “trivalent”, i.e., comprises or consists of three ISVDs. In another embodiment, the polypeptide is “tetravalent”, i.e. comprises or consists of four ISVDs. The polypeptide can thus be “bivalent”, “trivalent”, “tetravalent”, “pentavalent”, “hexavalent”, “heptavalent”, “octavalent”, “nonavalent”, etc., i.e., the polypeptide comprises or consists of two, three, four, five, six, seven, eight, nine, etc., ISVDs, respectively. In one embodiment the multivalent ISVD polypeptide is trivalent. In another embodiment the multivalent ISVD polypeptide is tetravalent. In still another embodiment, the multivalent ISVD polypeptide is pentavalent.

In one embodiment, the multivalent ISVD polypeptide can also be multispecific. The term “multispecific” refers to binding to multiple different target molecules (also referred to as antigens). The multivalent ISVD polypeptide can thus be “bispecific”, “trispecific”, “tetraspecific”, etc., i.e., can bind to two, three, four, etc., different target molecules, respectively.

For example, the polypeptide may be bispecific-trivalent, such as a polypeptide comprising or consisting of three ISVDs, wherein two ISVDs bind to a first target and one ISVD binds to a second target different from the first target. In another example, the polypeptide may be trispecific-tetravalent, such as a polypeptide comprising or consisting of four ISVDs, wherein one ISVD binds to a first target, two ISVDs bind to a second target different from the first target and one ISVD binds to a third target different from the first and the second target. In still another example, the polypeptide may be trispecific-pentavalent, such as a polypeptide comprising or consisting of five ISVDs, wherein two ISVDs bind to a first target, two ISVDs bind to a second target different from the first target and one ISVD binds to a third target different from the first and the second target.

In one embodiment, the multivalent ISVD polypeptide can also be multiparatopic. The term “multiparatopic” refers to binding to multiple different epitopes on the same target molecules (also referred to as antigens). The multivalent ISVD polypeptide can thus be “biparatopic”, “triparatopic”, etc., i.e., can bind to two, three, etc., different epitopes on the same target molecules, respectively.

In another aspect, the polypeptide of the present disclosure that comprises or essentially consists of one or more immunoglobulin single variable domains (or suitable fragments thereof), may further comprise one or more other groups, residues, moieties or binding units. Such further groups, residues, moieties, binding units or amino acid sequences may or may not provide further functionality to the immunoglobulin single variable domain (and/or to the polypeptide in which it is present) and may or may not modify the properties of the immunoglobulin single variable domain.

For example, such further groups, residues, moieties or binding units may be one or more additional amino acids, such that the compound, construct or polypeptide is a (fusion) protein or (fusion) polypeptide. In a preferred but non-limiting aspect, said one or more other groups, residues, moieties or binding units are immunoglobulins. Even more preferably, said one or more other groups, residues, moieties or binding units are chosen from the group consisting of domain antibodies, amino acids that are suitable for use as a domain antibody, single domain antibodies, amino acids that are suitable for use as a single domain antibody, “dAb” s, amino acids that are suitable for use as a dAb, VHHs (including humanized VHHs), VHs (including human VHs, camelized VHs and camelized human VHs), and VLs.

Alternatively, such groups, residues, moieties or binding units may for example be chemical groups, residues, moieties, which may or may not by themselves be biologically and/or pharmacologically active. For example, and without limitation, such groups may be linked to the one or more immunoglobulin single variable domain so as to provide a “derivative” of the immunoglobulin single variable domain.

In another embodiment, said further residues may be effective in preventing or reducing binding by so-called “pre-existing antibodies” to the polypeptides. For this purpose, the polypeptides and constructs may contain a C-terminal extension (X)n (in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I), for which reference is made to WO 2012/175741. Accordingly, the polypeptide may further comprise a C-terminal extension (X)n, in which n is 1 to 5, such as 1, 2, 3, 4 or 5, and in which X is a naturally occurring amino acid, preferably no cysteine.

In one embodiment, the polypeptide may further comprise one or more other groups, residues, moieties or binding units, optionally linked via one or more peptidic linkers, in which said one or more other groups, residues, moieties or binding units provide the polypeptide with increased (in vivo) half-life, compared to the corresponding polypeptide without said one or more other groups, residues, moieties or binding units. In vivo half-life extension means, for example, that the polypeptide has an increased half-life in a mammal, such as a human subject, after administration. Half-life can be expressed for example as t½beta.

The type of groups, residues, moieties or binding units is not generally restricted and may for example be chosen from the group consisting of a polyethylene glycol molecule, serum proteins or fragments thereof, binding units that can bind to serum proteins, an Fc portion, and small proteins or peptides that can bind to serum proteins.

More specifically, said one or more other groups, residues, moieties or binding units that provide the polypeptide with increased half-life can be chosen from the group consisting of binding units that can bind to serum albumin, such as human serum albumin, or a serum immunoglobulin, such as IgG. In one embodiment, said one or more other groups, residues, moieties or binding units that provide the polypeptide with increased half-life is a binding unit that can bind to human serum albumin. In one embodiment, the binding unit is an ISVD.

For example, WO 2004/041865 and WO 2006/122787 describes ISVDs binding to serum albumin (and in particular against human serum albumin) that can be linked to other proteins (such as one or more other ISVDs binding to a desired target) in order to increase the half-life of said protein. These ISVDs include the ISVDs called Alb-1 (SEQ ID NO: 52 in WO 2006/122787) and humanized variants thereof, such as Alb-8 (SEQ ID NO: 62 in WO 2006/122787). Again, these can be used to extend the half-life of therapeutic proteins and polypeptide and other therapeutic entities or moieties. Moreover, WO 2012/175400 describes a further improved version of Alb-1, called Alb-23.

In one embodiment, the polypeptide comprises a serum albumin binding moiety selected from Alb-1, Alb-3, Alb-4, Alb-5, Alb-6, Alb-7, Alb-8, Alb-9, Alb-10 (WO 2006/122787) and Alb-23. In one embodiment, the serum albumin binding moiety is Alb-8 or Alb-23 or its variants, as shown on pages 7-9 of WO 2012/175400. In one embodiment, the serum albumin binding moiety is selected from the albumin binders described in WO 2012/175741, WO2015/173325, WO2017/080850, WO2017/085172, WO2018/104444, WO2018/134235, and WO2018/134234. Some serum albumin binders are also shown in Table B. In one embodiment, the serum albumin binder is AlbX00001 (SEQ ID NO: 334). In one embodiment the serum albumin binder is Alb23002 (SEQ ID NO: 335).

In the polypeptides described above, the one or more immunoglobulin single variable domains and the one or more groups, residues, moieties or binding units may be linked directly to each other and/or via one or more suitable linkers or spacers. For example, when the one or more groups, residues, moieties or binding units are amino acids, the linkers may also be an amino acid, so that the resulting polypeptide is a fusion protein or fusion polypeptide.

As used herein, the term “linker” denotes a peptide that fuses together two or more ISVDs into a single molecule. The use of linkers to connect two or more (poly) peptides is well known in the art.

In some embodiments, the further exemplary peptide linkers are shown in Table B. One often used class of peptide linker are known as the “Gly-Ser” or “GS” linkers. These are linkers that essentially consist of glycine (G) and serine(S) residues, and usually comprise one or more repeats of a peptide motif such as the GGGGS (SEQ ID NO: 337) motif (for example, having the formula (Gly-Gly-Gly-Gly-Ser) n in which n may be 1, 2, 3, 4, 5, 6, 7 or more). Some often-used examples of such GS linkers are 9GS linkers (GGGGSGGGS, SEQ ID NO: 340), 15GS linkers (n=3) and 35GS linkers (n=7). Reference is for example made to Chen et al. 2013 (Adv. Drug Deliv. Rev. 65 (10): 1357-1369) and Klein et al. 2014 (Protein Eng. Des. Sel. 27 (10): 325-330).

TABLE B
Serum albumin binding ISVD sequences, Linker sequences and some proposed ISVD
C-terminal ends (amino acids 109-112 or 109-113, according to Kabat numbering)
(“ID” refers to the SEQ ID NO as used herein).

| Name Seq ID | Amino acid sequence |
|---|---|
| Alb8 | EVQLVESGGGLVQPGNSLRLSCAASGFTFSSFGMSWVRQAPGKGLEWVSSISGSGSDT |
| 320 | LYADSVKGR |
| | FTISRDNAKTTLYLQMNSLRPEDTAVYYCTIGGSLSRSSQGTLVTVSS |
| Alb23 | EVQLLESGGGLVQPGGSLRLSCAASGFTERSFGMSWVRQAPGKGPEWVSSISGSGSDT |
| 321 | LYADSVKGR |
| | FTISRDNSKNTLYLQMNSLRPEDTAVYYCTIGGSLSRSSQGTLVTVSS |
| Alb129 | EVQLVESGGGVVQPGNSLRLSCAASGFTESSFGMSWVRQAPGKGLEWVSSISGSGSDT |
| 322 | LYADSVKGR |
| | FTISRDNAKTTLYLQMNSLRPEDTATYYCTIGGSLSRSSQGTLVTVSSA |
| Alb132 | EVQLVESGGGVVQPGGSLRLSCAASGFTERSFGMSWVRQAPGKGPEWVSSISGSGSDT |
| 323 | LYADSVKGR |
| | FTISRDNSKNTLYLQMNSLRPEDTATYYCTIGGSLSRSSQGTLVTVSSA |
| Alb11 | EVQLVESGGGLVQPGNSLRLSCAASGFTESSFGMSWVRQAPGKGLEWVSSISGSGSDT |
| 324 | LYADSVKGR |
| | FTISRDNAKTTLYLQMNSLRPEDTAVYYCTIGGSLSRSSQGTLVTVSS |
| Alb11 | EVQLVESGGGLVQPGNSLRLSCAASGFTESSFGMSWVRQAPGKGLEWVSSISGSGSDT |
| (S112K)-A | LYADSVKGR |
| 325 | FTISRDNAKTTLYLQMNSLRPEDTAVYYCTIGGSLSRSSQGTLVKVSSA |
| Alb82 | EVQLVESGGGVVQPGNSLRLSCAASGFTESSFGMSWVRQAPGKGLEWVSSISGSGSDT |
| 326 | LYADSVKGR |
| | FTISRDNAKTTLYLQMNSLRPEDTALYYCTIGGSLSRSSQGTLVTVSS |
| Alb82-A | EVQLVESGGGVVQPGNSLRLSCAASGFTESSFGMSWVRQAPGKGLEWVSSISGSGSDT |
| 327 | LYADSVKGR |
| | FTISRDNAKTTLYLQMNSLRPEDTALYYCTIGGSLSRSSQGTLVTVSSA |
| Alb82-AA | EVQLVESGGGVVQPGNSLRLSCAASGFTESSFGMSWVRQAPGKGLEWVSSISGSGSDT |
| 328 | LYADSVKGR |
| | FTISRDNAKTTLYLQMNSLRPEDTALYYCTIGGSLSRSSQGTLVTVSSAA |
| Alb82-AAA | EVQLVESGGGVVQPGNSLRLSCAASGFTESSFGMSWVRQAPGKGLEWVSSISGSGSDT |
| 329 | LYADSVKGR |
| | FTISRDNAKTTLYLQMNSLRPEDTALYYCTIGGSLSRSSQGTLVTVSSAAA |
| Alb82-G | EVQLVESGGGVVQPGNSLRLSCAASGFTESSFGMSWVRQAPGKGLEWVSSISGSGSDT |
| 330 | LYADSVKGR |
| | FTISRDNAKTTLYLQMNSLRPEDTALYYCTIGGSLSRSSQGTLVTVSSG |
| Alb82-GG | EVQLVESGGGVVQPGNSLRLSCAASGFTESSFGMSWVRQAPGKGLEWVSSISGSGSDT |
| 331 | LYADSVKGR |
| | FTISRDNAKTTLYLQMNSLRPEDTALYYCTIGGSLSRSSQGTLVTVSSGG |
| Alb82-GGG | EVQLVESGGGVVQPGNSLRLSCAASGFTESSFGMSWVRQAPGKGLEWVSSISGSGSDT |
| 332 | LYADSVKGR |
| | FTISRDNAKTTLYLQMNSLRPEDTALYYCTIGGSLSRSSQGTLVTVSSGGG |
| Alb223 | EVQLVESGGGVVQPGGSLRLSCAASGFTFRSFGMSWVRQAPGKGPEWVSSISGSGSDT |
| 333 | LYADSVKGR |
| | FTISRDNSKNTLYLQMNSLRPEDTALYYCTIGGSLSRSSQGTLVTVSSA |
| AlbX00001 | EVQLVESGGGVVQPGGSLRLSCAASGLTFSSYAMGWFRQAPGKERERVVSISRGGGYT |
| 334 | YYADSVKGR |
| | FTISRDNSENTVYLQMNSLRPEDTALYYCAAARYWATGSEYEFDYWGQGTLVTVSS |
| Alb23002 | EVQLVESGGGVVQPGGSLRLSCAASGFTFRSFGMSWVRQAPGKGPEWVSSISGSGSDT |
| 335 | LYADSVKGR |
| | FTISRDNSKNTLYLQMNSLRPEDTALYYCTIGGSLSRSSQGTLVTVSS |
| 336 | AAA |
| 337 | GGGGS |
| 338 | SGGSGGS |
| 339 | GGGGSGGS |
| 340 | GGGGSGGGS |
| 341 | GGGGSGGGGS |
| 342 | GGGGGGGGSGGGGS |
| 343 | GGGGSGGGGSGGGGSGGS |
| 344 | GGGGSGGGGSGGGGSGGGGS |
| 345 | GGGGGGGGSGGGGSGGGGSGGGGS |
| 346 | GGGGSGGGGSGGGGSGGGGSGGGGSGGGGS |
| 347 | GGGGSGGGGSGGGGSGGGGSGGGGSGGGGSGGGGS |
| 348 | GGGGSGGGGSGGGGSGGGGSGGGGSGGGGSGGGGSGGGGS |
| 349 | EPKSCDKTHTCPPCP |
| 350 | GGGGSGGGSEPKSCDKTHTCPPCP |
| 351 | EPKTPKPQPAAA |
| 352 | ELKTPLGDTTHTCPRCPEPKSCDTPPPCPRCPEPKSCDTPPPCPRCPEPKSCDTPPPC |
| | PRCP |
| 353 | VTVSS(X)n |
| 354 | VKVSS |
| 355 | VQVSS |
| 356 | VTVKS |
| 357 | VTVQS |
| 358 | VKVKS |
| 359 | VKVQS |
| 360 | VQVKS |
| 361 | VQVQS |
| 362 | VTVSSA |
| 363 | VKVSSA |
| 364 | VQVSSA |
| 365 | VTVKSA |
| 366 | VTVQSA |
| 367 | VKVKSA |
| 368 | VKVQSA |
| 369 | VQVKSA |
| 370 | VQVQSA |
| 371 | VTVSS |

In one aspect, the disclosure relates to CD8 binders that can bind to and/or are directed against CD8. CD8 is a disulfide-linked dimer of α and β chain, two a chains, or two β chains (see, Weber and Cantor, Encyclopedia of Immunology, p475-478, 1998; Li et al., Front Immunol. 4:206, 2013). The most common form of CD8 is composed of a CD8-α and CD8-β chain. In mammals, the CD8 receptor acts as a co-receptor with the TCR complex. The CD8α and CD8β chains display high homology to immunoglobulin variable domains. The CD8αβ and CD8αα dimers bind to MHC class I molecules and help stabilize ligand binding during antigen presentation and T cell receptor activation. In some embodiments, the CD8 binder specifically recognizes a human CD8. In some embodiments, the CD8 binder specifically recognizes a cynomolgus monkey and/or rhesus macaque CD8. In some embodiments, the CD8 binder specifically recognizes both human and cynomolgus monkey and/or rhesus macaque CD8. CD8 as used herein may be from various animal species, including human, primate, mouse, rat, rabbit, or other mammals. CD8 as used herein can be wild-type or mutant (e.g., has insertion, deletion, and/or amino acid substitution compared to wild-type). In some embodiments, the CD8 binder specifically binds to the extracellular domain of one or both of CD8α and CD8β. In some embodiments, the CD8 binder specifically binds to the extracellular domain of CD8α. In some embodiments, the CD8 binder specifically binds to the extracellular domain of CD8β.

In some embodiments, the CD8 binder comprises an anti-CD8 antibody moiety (e.g., full-length, sdAb, scFv, or Fab) that specifically binds to both human and non-human primate (such as cynomolgus monkey and/or rhesus macaque) CD8. Exemplary anti-human CD8 antibodies and antigen binding fragments thereof with cross reactivity to human and monkey CD8 include, but are not limited to, cM-T807 mouse-human chimeric monoclonal antibody (see, for example, Schmitz, J. E. et al., Am J Pathol. 154(6):1923-1932, 1999), REGN5054 human monoclonal antibody (see, for example, Fredriksson, F. et al., J Nucl Med. 61(1): abstract 158), 3B5 mouse monoclonal antibody (see, for example, Woolridge, L. et al., J. Immunol. 171(12):6650-6660, 2003), LT8 mouse monoclonal antibody (see, for example, Sestak, K. et al., Vet Immunol Immunopathol. 119(1-2):21-26, 2007), FK18 mouse monoclonal antibody (see, for example, Stevens, H. P. J. D. et al., J Med Primatol. 20(8):386-393, 1991), or RPA-T8 monoclonal mouse antibody (see, for example, Bozkus, C. C. et al., STAR Protoc. 2(3):100758, 2021). Use of such anti-CD8 antibody moieties with cross-reactivity to monkey CD8 may facilitate toxicity studies in non-human primates, which can provide more relevant safety assessments for human clinical trial candidates, without having to perform toxicity studies in chimpanzees or using surrogate molecules.

In some embodiments, the anti-CD8 antibody moiety (e.g., full-length, sdAb, scFv, or Fab) is derived from an anti-CD8 antibody or antigen binding fragment thereof that does not have cross-reactivity to non-human primates. Such exemplary anti-CD8 antibody moieties include, but are not limited to, the QA18A37 mouse monoclonal antibody (see, for example, Paulikat, A. D. et al. J Innate Immun. 14(5):569-580, 2022), MEM-31 mouse monoclonal antibody (see, for example, Horejsi, V. et al., Folia Biol. 34(1):23-24, 1988), UCHT-4 mouse monoclonal antibody (see, for example, Willemsen, R. A. et al. J Immunol. 177(2):991-998, 2006), and OKT8 mouse monoclonal antibody (see, for example, Shiratsuchi, H. and Tsuyuguchi, I., Clin Exp Immunol. 57(2):271-278, 1984). Further CD8 binding molecules contemplated herein include CD8 binding molecules described in U.S. Pat. No. 11,535,869; WO2021/046159; WO2019/023148; WO2019/033043; WO1993/008817; WO2014/164553; the contents of each of which are incorporated herein by reference in their entirety.

In one aspect, the disclosure also relates to ISVDs as described herein that can bind to and/or are directed against CD8α (CD8alpha) and that comprise CDR sequences that are generally as further defined herein, to suitable fragments thereof, as well as to polypeptides that comprise or essentially consist of one or more of such ISVDs and/or suitable fragments. In some aspect, the disclosure relates to an ISVDs comprising a sequence selected from the group consisting of SEQ ID NOs: 160 to 179. In particular, the disclosure in some specific aspects provides:
(I) ISVDs that are directed against CD8α and that have at least 80%, preferably at least 85%, such as 90% or 95% or more sequence identity with an ISVD comprising a sequence selected from the group consisting of SEQ ID NOs: 160 to 179;
(II) ISVDs that cross-block the binding of the amino acid sequence selected from the group consisting of SEQ ID NOs: 160 to 179 to CD8α and/or that compete with at least the ISVD selected from the group consisting of SEQ ID NOs: 160 to 179 for binding to CD8a.

Such ISVDs may be as further described herein (and may for example be VHHs, including humanized VHHs, VHs, including human VHs, camelized VHs and camelized human VHs); as well as polypeptides of the disclosure that comprise one or more of such amino acid sequences (which may be as further described herein), and particularly bispecific (or multispecific) polypeptides as described herein, and nucleic acid sequences that encode ISVDs and polypeptides. Such ISVDs and polypeptides do not include any naturally occurring ligands.

In some embodiments, the CD8α is derived from a mammalian animal, such as a human being. In one specific, but non-limiting aspect, the disclosure relates to an ISVD directed against CD8α, that comprises:
a) the amino acid sequence selected from the group consisting of SEQ ID NOs: 160-179;
b) amino acid sequences that have at least 80% amino acid identity with a sequence selected from the group consisting of SEQ ID NOs: 160-179, or
c) amino acid sequences that have 3, 2, or 1 amino acid difference with a sequence selected from the group consisting of SEQ ID NOs: 160-179;
or any suitable combination thereof.

In some embodiments, disclosed is an ISVD against CD8a, which consist of 4 framework regions (FR1 to FR4 respectively) and 3 complementarity determining regions (CDR1 to CDR3 respectively). In some embodiments, in such an ISVD:
(I) CDR1 comprises or essentially consists of an amino acid sequence selected from the group consisting of SEQ ID NOs: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering; or amino acid sequences that have at least 80%, at least 90%, at least 95%, at least 99% or more sequence identity with a sequence selected from the group consisting of SEQ ID NO: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering; and/or amino acids sequences that have 2 or only 1 amino acid difference(s) with a sequence selected from the group consisting of SEQ ID NO: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering, in which any amino acid substitution is a conservative amino acid substitution; and/or said amino acid sequence only contains amino acid substitutions, and no amino acid deletions or insertions, compared to a sequence selected from the group consisting of SEQ ID NO: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering.
(II) CDR2 comprises or essentially consists of an amino acid sequence comprising a sequence selected from the group consisting of SEQ ID NO: 183, 190, 197, 204, 211, 218, 225, 232, 239 and 246 according to Abm numbering, and SEQ ID NOs: 253, 260, 267, 274, 281, 288, 295, 302, 309 and 316 according to Kabat numbering, or amino acid sequences that have at least 80%, at least 90%, at least 95%, at least 99% or more sequence identity with a sequence selected from the group consisting of SEQ ID NO: 183, 190, 197, 204, 211, 218, 225, 232, 239 and 246 according to Abm numbering, and SEQ ID NOs: 253, 260, 267, 274, 281, 288, 295, 302, 309 and 316 according to Kabat numbering in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 183, 190, 197, 204, 211, 218, 225, 232, 239 and 246 according to Abm numbering, and SEQ ID NOs: 253, 260, 267, 274, 281, 288, 295, 302, 309 and 316 according to Kabat numbering; and/or amino acids sequences that have 2 or only 1 amino acid difference(s) with a sequence selected from the group consisting of SEQ ID NO: 183, 190, 197, 204, 211, 218, 225, 232, 239 and 246 according to Abm numbering, and SEQ ID NOs: 253, 260, 267, 274, 281, 288, 295, 302, 309 and 316 according to Kabat numbering in which any amino acid substitution is a conservative amino acid substitution; and/or said amino acid sequence only contains amino acid substitutions, and no amino acid deletions or insertions, compared to a sequence selected from the group consisting of SEQ ID NO: 183, 190, 197, 204, 211, 218, 225, 232, 239 and 246 according to Abm numbering and SEQ ID NOs: 253, 260, 267, 274, 281, 288, 295, 302, 309 and 316 according to Kabat numbering.
(III) CDR3 comprises or essentially consists of an amino acid sequence comprising a sequence selected from the group consisting of SEQ ID NO: 185, 192, 199, 206, 213, 220, 227, 234, 241 and 248 according to Abm numbering, and SEQ ID NOs: 255, 262, 269, 276, 283, 290, 297, 304, 311 and 318 according to Kabat numbering, or amino acid sequences that have at least 80%, at least 90%, at least 95%, at least 99% or more sequence identity with a sequence selected from the group consisting of SEQ ID NO: 185, 192, 199, 206, 213, 220, 227, 234, 241 and 248 according to Abm numbering and SEQ ID NOs: 255, 262, 269, 276, 283, 290, 297, 304, 311 and 318 according to Kabat numbering, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 185, 192, 199, 206, 213, 220, 227, 234, 241 and 248 according to Abm numbering and SEQ ID NOs: 255, 262, 269, 276, 283, 290, 297, 304, 311 and 318 according to Kabat numbering; and/or from the group consisting of amino acids sequences that have 2 or only 1 amino acid difference(s) with a sequence selected from the group consisting of SEQ ID NO: 185, 192, 199, 206, 213, 220, 227, 234, 241 and 248 according to Abm numbering and SEQ ID NOs: 255, 262, 269, 276, 283, 290, 297, 304, 311 and 318 according to Kabat numbering, in which any amino acid substitution is a conservative amino acid substitution; and/or said amino acid sequence only contains amino acid substitutions, and no amino acid deletions or insertions, compared to a sequence selected from the group consisting of SEQ ID NO: 185, 192, 199, 206, 213, 220, 227, 234, 241 and 248 according to Abm numbering and SEQ ID NOs: 4 255, 262, 269, 276, 283, 290, 297, 304, 311 and 318 according to Kabat numbering.

In one embodiment, the ISVD comprises a CDR1 that is the amino acid sequence of SEQ ID NO: 244, a CDR2 that is the amino acid sequence of SEQ ID NO: 246 and a CDR3 that is the amino acid sequence of SEQ ID NO: 248 (Abm numbering) or that comprises a CDR1 that is the amino acid sequence of SEQ ID NO: 314, a CDR2 that is the amino acid sequence of SEQ ID NO: 316 and a CDR3 that is the amino acid sequence of SEQ ID NO: 318 (Kabat numbering).

CD8 binding ISVDs as disclosed herein may comprise one, two or all three of the CDRs explicitly listed above. In some embodiments, the anti-CD8α ISVD is selected from the ISVDs described in the tables below. In some embodiments, the anti-CD8αISVD is selected from the ISVDs that have at least 80%, at least 85%, at least 90%, at least 95%, at least 96%, at least 97%, at least 98%, or at least 99% sequence identity with the ISVDs described Table C-1, Table C-2, and Table C-3. In some embodiments, the anti-CD8 ISVD is selected from the ISVDs that have at least 80%, at least 85%, at least 90%, at least 95%, at least 96%, at least 97%, at least 98%, or at least 99% sequence identity with the ISVDs described in Table C-4.

TABLE C-1
Anti-CD8α ISVDs.

| | Full | fw 1 | cdr1 | fw 2 | cdr2 | fw3 | cdr3 | fw4 |
|---|---|---|---|---|---|---|---|---|
| | SEQ | SEQ | SEQ | SEQ | SEQ | SEQ | SEQ | SEQ |
| name | ID | ID | ID | ID | ID | ID | ID | ID |
| Abm CDR | | | | | | | | |
| definition | | | | | | | | |
| T0347015C01 | 160 | 180 | 181 | 182 | 183 | 184 | 185 | 186 |
| (15C01) | | | | | | | | |
| A044300805 | 161 | 187 | 188 | 189 | 190 | 191 | 192 | 193 |
| A044300805_v1 | 162 | 194 | 195 | 196 | 197 | 198 | 199 | 200 |
| (15C01v1) | | | | | | | | |
| A044300805_v2 | 163 | 201 | 202 | 203 | 204 | 205 | 206 | 207 |
| (15C01v2) | | | | | | | | |
| A044300805_v3 | 164 | 208 | 209 | 210 | 211 | 212 | 213 | 214 |
| (15C01v3) | | | | | | | | |
| A044300805_v4 | 165 | 215 | 216 | 217 | 218 | 219 | 220 | 221 |
| (15C01v4) | | | | | | | | |
| A044300805_v5 | 166 | 222 | 223 | 224 | 225 | 226 | 227 | 228 |
| (15C01v5) | | | | | | | | |
| A044300805_v6 | 167 | 229 | 230 | 231 | 232 | 233 | 234 | 235 |
| (15C01v6) | | | | | | | | |
| A044300805_v7 | 168 | 236 | 237 | 238 | 239 | 240 | 241 | 242 |
| (15C01v7) | | | | | | | | |
| A044300805_v8 | 169 | 243 | 244 | 245 | 246 | 247 | 248 | 249 |
| (15C01v8) | | | | | | | | |
| Kabat CDR | | | | | | | | |
| definition | | | | | | | | |
| T0347015C01 | 170 | 250 | 251 | 252 | 253 | 254 | 255 | 256 |
| (15C01) | | | | | | | | |
| A044300805 | 171 | 257 | 258 | 259 | 260 | 261 | 262 | 263 |
| A044300805_v1 | 172 | 264 | 265 | 266 | 267 | 268 | 269 | 270 |
| (15C01v1) | | | | | | | | |
| A044300805_v2 | 173 | 271 | 272 | 273 | 274 | 275 | 276 | 277 |
| (15C01v2) | | | | | | | | |
| A044300805_v3 | 174 | 278 | 279 | 280 | 281 | 282 | 283 | 284 |
| (15C01v3) | | | | | | | | |
| A044300805_v4 | 175 | 285 | 286 | 287 | 288 | 289 | 290 | 291 |
| (15C01v4) | | | | | | | | |
| A044300805_v5 | 176 | 292 | 293 | 294 | 295 | 296 | 297 | 298 |
| (15C01v5) | | | | | | | | |
| A044300805_v6 | 177 | 299 | 300 | 301 | 302 | 303 | 304 | 305 |
| (15C01v6) | | | | | | | | |
| A044300805_v7 | 178 | 306 | 307 | 308 | 309 | 310 | 311 | 312 |
| (15C01v7) | | | | | | | | |
| A044300805_v8 | 179 | 313 | 314 | 315 | 316 | 317 | 318 | 319 |
| (15C01v8) | | | | | | | | |
| IMGT CDR | | | | | | | | |
| definition | | | | | | | | |
| BDSn | 419 | 420 | 421 | 422 | 423 | 424 | 425 | 426 |

TABLE C-2
Anti-CD8α ISVD full sequences

| Seq | |
|---|---|
| ID | Full seq |
| 160 | EVQLVESGGGLVQAGGSLRLSCAASGFTEDDYAIGWFRQAPGKEREEVSCIRTYDGNTYYIDS |
| | VKGRFTISSDNAKNTVYLQMNSLKPEDTAAYYCAAGSYYACAKYSRPDPSENHVDMDYWGKGT |
| | LVTVSS |
| 161 | EVQLVESGGGVVQPGGSLRLSCAASGFTFEDYAIGWFRQAPGKEREEVSCIRTYDEQTYYADS |
| | VKGRFTISRDNAKNTVYLQMNSLRPEDTALYYCAAGSYYACAKYSRPDPSEGHVDLDYWGQGT |
| | LVTVSS |
| 162 | EVQLVESGGGVVQPGGSLRLSCAASGFTFEDYAIGWFRQAPGKEREEVSCIRTYDEQTYYADS |
| | VKGRFTISRDNAKNTVYLQMNSLRPEDTALYYCAAGSYYACAEYSRPDPSEGHVDLDYWGQGT |
| | LVTVSS |
| 163 | EVQLVESGGGVVQPGGSLRLSCAASGFTFEDYAIGWFRQAPGKEREEVSCIRTYDEQTYYADS |
| | VKGRFTISRDNAKNTVYLQMNSLRPEDTALYYCAAGSYYACAYYSRPDPSEGHVDLDYWGQGT |
| | LVTVSS |
| 164 | EVQLVESGGGVVQPGGSLRLSCAASGFTFEDYAIGWFRQAPGKEREEVSCIRTYDEQTYYADS |
| | VKGRFTISRDNAKNTVDLQMNSLRPEDTALYYCAAGSYYACAKYSRPDPSEGHVDLDYWGQGT |
| | LVTVSS |
| 165 | EVQLVESGGGVVQPGGSLRLSCAASGFTFEDYAIGWFRQAPGKEREEVSCIRTYDEQTYYADS |
| | VKGRFTISRDNAKNTVDLQMNSLRPEDTALYYCAAGSYYACAEYSRPDPSEGHVDLDYWGQGT |
| | LVTVSS |
| 166 | EVQLVESGGGVVQPGGSLRLSCAASGFTFEDYAIGWFRQAPGKEREEVSCIRTYDEQTYYADS |
| | VKGRFTISRDNAKNTVDLQMNSLRPEDTALYYCAAGSYYACAYYSRPDPSEGHVDLDYWGQGT |
| | LVTVSS |
| 167 | EVQLVESGGGVVQPGGSLRLSCAASGFTFEDYAIGWFRQAPGKEREEVSCIRTYDEQTYYADS |
| | VKGRFTISRDNAKNTVSLQMNSLRPEDTALYYCAAGSYYACAKYSRPDPSEGHVDLDYWGQGT |
| | LVTVSS |
| 168 | EVQLVESGGGVVQPGGSLRLSCAASGFTFEDYAIGWFRQAPGKEREEVSCIRTYDEQTYYADS |
| | VKGRFTISRDNAKNTVSLQMNSLRPEDTALYYCAAGSYYACAEYSRPDPSEGHVDLDYWGQGT |
| | LVTVSS |
| 169 | EVQLVESGGGVVQPGGSLRLSCAASGFTFEDYAIGWFRQAPGKEREEVSCIRTYDEQTYYADS |
| | VKGRFTISRDNAKNTVSLQMNSLRPEDTALYYCAAGSYYACAYYSRPDPSEGHVDLDYWGQGT |
| | LVTVSS |
| 170 | EVQLVESGGGLVQAGGSLRLSCAASGFTEDDYAIGWFRQAPGKEREEVSCIRTYDGNTYYIDS |
| | VKGRFTISSDNAKNTVYLQMNSLKPEDTAAYYCAAGSYYACAKYSRPDPSENHVDMDYWGKGT |
| | LVTVSS |
| 171 | EVQLVESGGGVVQPGGSLRLSCAASGFTFEDYAIGWFRQAPGKEREEVSCIRTYDEQTYYADS |
| | VKGRFTISRDNAKNTVYLQMNSLRPEDTALYYCAAGSYYACAKYSRPDPSEGHVDLDYWGQGT |
| | LVTVSS |
| 172 | EVQLVESGGGVVQPGGSLRLSCAASGFTFEDYAIGWFRQAPGKEREEVSCIRTYDEQTYYADS |
| | VKGRFTISRDNAKNTVYLQMNSLRPEDTALYYCAAGSYYACAEYSRPDPSEGHVDLDYWGQGT |
| | LVTVSS |
| 173 | EVQLVESGGGVVQPGGSLRLSCAASGFTFEDYAIGWFRQAPGKEREEVSCIRTYDEQTYYADS |
| | VKGRFTISRDNAKNTVYLQMNSLRPEDTALYYCAAGSYYACAYYSRPDPSEGHVDLDYWGQGT |
| | LVTVSS |
| 174 | EVQLVESGGGVVQPGGSLRLSCAASGFTFEDYAIGWFRQAPGKEREEVSCIRTYDEQTYYADS |
| | VKGRFTISRDNAKNTVDLQMNSLRPEDTALYYCAAGSYYACAKYSRPDPSEGHVDLDYWGQGT |
| | LVTVSS |
| 175 | EVQLVESGGGVVQPGGSLRLSCAASGFTFEDYAIGWFRQAPGKEREEVSCIRTYDEQTYYADS |
| | VKGRFTISRDNAKNTVDLQMNSLRPEDTALYYCAAGSYYACAEYSRPDPSEGHVDLDYWGQGT |
| | LVTVSS |
| 176 | EVQLVESGGGVVQPGGSLRLSCAASGFTFEDYAIGWERQAPGKEREEVSCIRTYDEQTYYADS |
| | VKGRFTISRDNAKNTVDLQMNSLRPEDTALYYCAAGSYYACAYYSRPDPSEGHVDLDYWGQGT |
| | LVTVSS |
| 177 | EVQLVESGGGVVQPGGSLRLSCAASGFTFEDYAIGWFRQAPGKEREEVSCIRTYDEQTYYADS |
| | VKGRFTISRDNAKNTVSLQMNSLRPEDTALYYCAAGSYYACAKYSRPDPSEGHVDLDYWGQGT |
| | LVTVSS |
| 178 | EVQLVESGGGVVQPGGSLRLSCAASGFTFEDYAIGWFRQAPGKEREEVSCIRTYDEQTYYADS |
| | VKGRFTISRDNAKNTVSLQMNSLRPEDTALYYCAAGSYYACAEYSRPDPSEGHVDLDYWGQGT |
| | LVTVSS |
| 179 | EVQLVESGGGVVQPGGSLRLSCAASGFTFEDYAIGWFRQAPGKEREEVSCIRTYDEQTYYADS |
| | VKGRFTISRDNAKNTVSLQMNSLRPEDTALYYCAAGSYYACAYYSRPDPSEGHVDLDYWGQGT |
| | LVTVSS |
| 28 | EVQLVESGGGLVQAGGSLRLSCAASGFTEDDYAIGWERQAPGKEREEVSCIRTYDGNTYYIDS |
| | VKGRFTISSDNAKNTVYLQMNSLKPEDTAAYYCAAGSYYACAKYSRPDPSENHVDMDYWGKGT |
| | LVTVSSGGC |
| 29 | EVQLVESGGGVVQPGGSLRLSCAASGFTFEDYAIGWERQAPGKEREEVSCIRTYDEQTYYADS |
| | VKGRFTISRDNAKNTVYLQMNSLRPEDTALYYCAAGSYYACAKYSRPDPSEGHVDLDYWGQGT |
| | LVTVSSGGC |
| 30 | EVQLVESGGGVVQPGGSLRLSCAASGFTFEDYAIGWFRQAPGKEREEVSCIRTYDEQTYYADS |
| | VKGRFTISRDNAKNTVYLQMNSLRPEDTALYYCAAGSYYACAEYSRPDPSEGHVDLDYWGQGT |
| | LVTVSSGGC |
| 31 | EVQLVESGGGVVQPGGSLRLSCAASGFTFEDYAIGWFRQAPGKEREEVSCIRTYDEQTYYADS |
| | VKGRFTISRDNAKNTVYLQMNSLRPEDTALYYCAAGSYYACAYYSRPDPSEGHVDLDYWGQGT |
| | LVTVSSGGC |
| 32 | EVQLVESGGGVVQPGGSLRLSCAASGFTFEDYAIGWFRQAPGKEREEVSCIRTYDEQTYYADS |
| | VKGRFTISRDNAKNTVDLQMNSLRPEDTALYYCAAGSYYACAKYSRPDPSEGHVDLDYWGQGT |
| | LVTVSSGGC |
| 33 | EVQLVESGGGVVQPGGSLRLSCAASGFTFEDYAIGWFRQAPGKEREEVSCIRTYDEQTYYADS |
| | VKGRFTISRDNAKNTVDLQMNSLRPEDTALYYCAAGSYYACAEYSRPDPSEGHVDLDYWGQGT |
| | LVTVSSGGC |
| 34 | EVQLVESGGGVVQPGGSLRLSCAASGFTFEDYAIGWFRQAPGKEREEVSCIRTYDEQTYYADS |
| | VKGRFTISRDNAKNTVDLQMNSLRPEDTALYYCAAGSYYACAYYSRPDPSEGHVDLDYWGQGT |
| | LVTVSSGGC |
| 35 | EVQLVESGGGVVQPGGSLRLSCAASGFTFEDYAIGWFRQAPGKEREEVSCIRTYDEQTYYADS |
| | VKGRFTISRDNAKNTVSLQMNSLRPEDTALYYCAAGSYYACAKYSRPDPSEGHVDLDYWGQGT |
| | LVTVSSGGC |
| 36 | EVQLVESGGGVVQPGGSLRLSCAASGFTFEDYAIGWFRQAPGKEREEVSCIRTYDEQTYYADS |
| | VKGRFTISRDNAKNTVSLQMNSLRPEDTALYYCAAGSYYACAEYSRPDPSEGHVDLDYWGQGT |
| | LVTVSSGGC |
| 44 | EVQLVESGGGVVQPGGSLRLSCAASGFTFEDYAIGWFRQAPGKEREEVSCIRTYDEQTYYADS |
| | VKGRFTISRDNAKNTVSLQMNSLRPEDTALYYCAAGSYYACAYYSRPDPSEGHVDLDYWGQGT |
| | LVTVSSGGC |

TABLE C-3
anti-CD8α ISVD CDRs.

| SEQ | | Seq | | Seq | | Seq | | Seq | | Seq | | Seq | |
|---|---|---|---|---|---|---|---|---|---|---|---|---|---|
| ID | Fw1 | ID | cdr1 | ID | fw2 | ID | cdr2 | ID | fw3 | ID | cdr3 | ID | fw4 |
| 180 | EVQLVESGG | 181 | GFT | 182 | WFRQ | 183 | CIRTY | 184 | YIDSVKGRFTISS | 185 | GSYYACA | 186 | WGK |
| | GLVQAGGSL | | FDD | | APGK | | DGNTY | | DNAKNTVYLQM | | KYSRPDPS | | GTL |
| | RLSCAAS | | YAI | | EREE | | | | NSLKPEDTAAYY | | ENHVDM | | VTV |
| | | | G | | VS | | | | CAA | | DY | | SS |
| 187 | EVQLVESGG | 188 | GFT | 189 | WFRQ | 190 | CIRTY | 191 | YADSVKGRFTISR | 192 | GSYYACA | 193 | WGQ |
| | GVVQPGGSL | | FED | | APGK | | DEQTY | | DNAKNTVYLQM | | KYSRPDPS | | GTL |
| | RLSCAAS | | YAI | | EREE | | | | NSLRPEDTALYY | | EGHVDLD | | VTV |
| | | | G | | VS | | | | CAA | | Y | | SS |
| 194 | EVQLVESGG | 195 | GFT | 196 | WFRQ | 197 | CIRTY | 198 | YADSVKGRFTISR | 199 | GSYYACA | 200 | WGQ |
| | GVVQPGGSL | | FED | | APGK | | DEQTY | | DNAKNTVYLQM | | EYSRPDPS | | GTL |
| | RLSCAAS | | YAI | | EREE | | | | NSLRPEDTALYY | | EGHVDLD | | VTV |
| | | | G | | VS | | | | CAA | | Y | | SS |
| 201 | EVQLVESGG | 202 | GFT | 203 | WFRQ | 204 | CIRTY | 205 | YADSVKGRFTISR | 206 | GSYYACA | 207 | WGQ |
| | GVVQPGGSL | | FED | | APGK | | DEQTY | | DNAKNTVYLQM | | YYSRPDPS | | GTL |
| | RLSCAAS | | YAI | | EREE | | | | NSLRPEDTALYY | | EGHVDLD | | VTV |
| | | | G | | VS | | | | CAA | | Y | | SS |
| 208 | EVQLVESGG | 209 | GFT | 210 | WFRQ | 211 | CIRTY | 212 | YADSVKGRFTISR | 213 | GSYYACA | 214 | WGQ |
| | GVVQPGGSL | | FED | | APGK | | DEQTY | | DNAKNTVDLQM | | KYSRPDPS | | GTL |
| | RLSCAAS | | YAI | | EREE | | | | NSLRPEDTALYY | | EGHVDLD | | VTV |
| | | | G | | VS | | | | CAA | | Y | | SS |
| 215 | EVQLVESGG | 216 | GFT | 217 | WFRQ | 218 | CIRTY | 219 | YADSVKGRFTISR | 220 | GSYYACA | 221 | WGQ |
| | GVVQPGGSL | | FED | | APGK | | DEQTY | | DNAKNTVDLQM | | EYSRPDPS | | GTL |
| | RLSCAAS | | YAI | | EREE | | | | NSLRPEDTALYY | | EGHVDLD | | VTV |
| | | | G | | VS | | | | CAA | | Y | | SS |
| 222 | EVQLVESGG | 223 | GFT | 224 | WFRQ | 225 | CIRTY | 226 | YADSVKGRFTISR | 227 | GSYYACA | 228 | WGQ |
| | GVVQPGGSL | | FED | | APGK | | DEQTY | | DNAKNTVDLQM | | YYSRPDPS | | GTL |
| | RLSCAAS | | YAI | | EREE | | | | NSLRPEDTALYY | | EGHVDLD | | VTV |
| | | | G | | VS | | | | CAA | | Y | | SS |
| 229 | EVQLVESGG | 230 | GFT | 231 | WFRQ | 232 | CIRTY | 233 | YADSVKGRFTISR | 234 | GSYYACA | 235 | WGQ |
| | GVVQPGGSL | | FED | | APGK | | DEQTY | | DNAKNTVSLQMN | | KYSRPDPS | | GTL |
| | RLSCAAS | | YAI | | EREE | | | | SLRPEDTALYYC | | EGHVDLD | | VTV |
| | | | G | | VS | | | | AA | | Y | | SS |
| 236 | EVQLVESGG | 237 | GFT | 238 | WFRQ | 239 | CIRTY | 240 | YADSVKGRFTISR | 241 | GSYYACA | 242 | WGQ |
| | GVVQPGGSL | | FED | | APGK | | DEQTY | | DNAKNTVSLQMN | | EYSRPDPS | | GTL |
| | RLSCAAS | | YAI | | EREE | | | | SLRPEDTALYYC | | EGHVDLD | | VTV |
| | | | G | | VS | | | | AA | | Y | | SS |
| 243 | EVQLVESGG | 244 | GFT | 245 | WFRQ | 246 | CIRTY | 247 | YADSVKGRFTISR | 248 | GSYYACA | 249 | WGQ |
| | GVVQPGGSL | | FED | | APGK | | DEQTY | | DNAKNTVSLQMN | | YYSRPDPS | | GTL |
| | RLSCAAS | | YAI | | EREE | | | | SLRPEDTALYYC | | EGHVDLD | | VTV |
| | | | G | | VS | | | | AA | | Y | | SS |
| 250 | EVQLVESGG | 251 | DY | 252 | WFRQ | 253 | CIRTY | 254 | RFTISSDNAKNTV | 255 | GSYYACA | 256 | WGK |
| | GLVQAGGSL | | AIG | | APGK | | DGNTY | | YLQMNSLK | | KYSRPDPS | | GTL |
| | RLSCAASGFT | | | | EREE | | YIDSV | | PEDTAAYYCAA | | ENHVDM | | VTV |
| | FD | | | | VS | | KG | | | | DY | | SS |
| 257 | EVQLVESGG | 258 | DY | 259 | WFRQ | 260 | CIRTY | 261 | RFTISRDNAKNTV | 262 | GSYYACA | 263 | WGQ |
| | GVVQPGGSL | | AIG | | APGK | | DEQTY | | YLQMNSLR | | KYSRPDPS | | GTL |
| | RLSCAASGFT | | | | EREE | | YADSV | | PEDTALYYCAA | | EGHVDLD | | VTV |
| | FE | | | | VS | | KG | | | | Y | | SS |
| 264 | EVQLVESGG | 265 | DY | 266 | WFRQ | 267 | CIRTY | 268 | RFTISRDNAKNTV | 269 | GSYYACA | 270 | WGQ |
| | GVVQPGGSL | | AIG | | APGK | | DEQTY | | YLQMNSLR | | EYSRPDPS | | GTL |
| | RLSCAASGFT | | | | EREE | | YADSV | | PEDTALYYCAA | | EGHVDLD | | VTV |
| | FE | | | | VS | | KG | | | | Y | | SS |
| 271 | EVQLVESGG | 272 | DY | 273 | WFRQ | 274 | CIRTY | 275 | RFTISRDNAKNTV | 276 | GSYYACA | 277 | WGQ |
| | GVVQPGGSL | | AIG | | APGK | | DEQTY | | YLQMNSLR | | YYSRPDPS | | GTL |
| | RLSCAASGFT | | | | EREE | | YADSV | | PEDTALYYCAA | | EGHVDLD | | VTV |
| | FE | | | | VS | | KG | | | | Y | | SS |
| 278 | EVQLVESGG | 279 | DY | 280 | WFRQ | 281 | CIRTY | 282 | RFTISRDNAKNTV | 283 | GSYYACA | 284 | WGQ |
| | GVVQPGGSL | | AIG | | APGK | | DEQTY | | DLQMNSLR | | KYSRPDPS | | GTL |
| | RLSCAASGFT | | | | EREE | | YADSV | | PEDTALYYCAA | | EGHVDLD | | VTV |
| | FE | | | | VS | | KG | | | | Y | | SS |
| 285 | EVQLVESGG | 286 | DY | 287 | WFRQ | 288 | CIRTY | 289 | RFTISRDNAKNTV | 290 | GSYYACA | 291 | WGQ |
| | GVVQPGGSL | | AIG | | APGK | | DEQTY | | DLQMNSLR | | EYSRPDPS | | GTL |
| | RLSCAASGFT | | | | EREE | | YADSV | | PEDTALYYCAA | | EGHVDLD | | VTV |
| | FE | | | | VS | | KG | | | | Y | | SS |
| 292 | EVQLVESGG | 293 | DY | 294 | WFRQ | 295 | CIRTY | 296 | RFTISRDNAKNTV | 297 | GSYYACA | 298 | WGQ |
| | GVVQPGGSL | | AIG | | APGK | | DEQTY | | DLQMNSLR | | YYSRPDPS | | GTL |
| | RLSCAASGFT | | | | EREE | | YADSV | | PEDTALYYCAA | | EGHVDLD | | VTV |
| | FE | | | | VS | | KG | | | | Y | | SS |
| 299 | EVQLVESGG | 300 | DY | 301 | WFRQ | 302 | CIRTY | 303 | RFTISRDNAKNTV | 304 | GSYYACA | 305 | WGQ |
| | GVVQPGGSL | | AIG | | APGK | | DEQTY | | SLQMNSLR | | KYSRPDPS | | GTL |
| | RLSCAASGFT | | | | EREE | | YADSV | | PEDTALYYCAA | | EGHVDLD | | VTV |
| | FE | | | | VS | | KG | | | | Y | | SS |
| 306 | EVQLVESGG | 307 | DY | 308 | WFRQ | 309 | CIRTY | 310 | RFTISRDNAKNTV | 311 | GSYYACA | 312 | WGQ |
| | GVVQPGGSL | | AIG | | APGK | | DEQTY | | SLQMNSLR | | EYSRPDPS | | GTL |
| | RLSCAASGFT | | | | EREE | | YADSV | | PEDTAL YYCAA | | EGHVDLD | | VTV |
| | FE | | | | VS | | KG | | | | Y | | SS |
| 313 | EVQLVESGG | 314 | DY | 315 | WFRQ | 316 | YADSV | 317 | RFTISRDNAKNTV | 318 | GSYYACA | 319 | WGQ |
| | GVVQPGGSL | | AIG | | APG | | KGC | | SLQMNSLR | | YYSRPDPS | | GTL |
| | RLSCAASGFT | | | | KERE | | IRTYD | | PEDTALYYCAA | | EGHVDLD | | VTV |
| | FE | | | | EVS | | EQTY | | | | Y | | SS |

In some embodiments, the ISVDs as described herein may further comprise additional amino acids at either the N-terminus and/or the C-terminus. In some embodiments, the additional amino acids are at the C-terminus. In some embodiments, the additional amino acids form a tag peptide that can facilitate purification. In some embodiments, the tag peptide is a poly-histidine (such as 6× His). In some embodiments, the additional amino acids can form a tag for downstream modification of the ISVD, such as a tag for conjugation reaction. In some embodiments, the tag for conjugation reaction comprises at least one cysteine, which can help with a downstream cysteine-based click chemistry reaction. In some embodiments, the tag for click chemistry reaction comprises GGC. In some embodiments, the additional amino acid can form both a tag for click chemistry, and a tag that can facilitate purification. In some embodiments, the tag for click chemistry reaction is GGC. For example, GGC can be added to the C-terminus of an ISVD (e.g. an anti-CD8α ISVD) as described herein, such as any ISVD of SEQ ID Nos: 160 to 179, to form new ISVD of SEQ ID Nos: 28-36 and 44. In some embodiments, the phospholipid-PEG-anti-CD8α conjugate is DSPE-PEG3.4K-A044300805_v8_GGC (a.k.a., T0347015C₀₁v8, SEQ ID NO: 44).

In some embodiments, the ISVDs as described herein comprise at least one internal disulfide bridge. In some embodiments, the ISVDs as described herein comprise two internal disulfide bridge, such as a canonical disulfide bridge and a VHH-specific bridge (e.g., a VHH1 specific disulfide bridge). In some embodiments, the ISVD is a VHH1-type ISVD. In some embodiments, the VHH1-type ISVD has two internal disulfide bridges (e.g., 1×canonical+1×exposed VHH1-specific), and one free cysteine for conjugation at the C-terminus (e.g., a GGC linker).

In the ISVDs of the disclosure that comprise the combinations of CDRs mentioned above, each CDR can be replaced by a CDR chosen from the group consisting of amino acid sequences that have at least 80%, preferably at least 90%, more preferably at least 95%, even more preferably at least 99% sequence identity with the mentioned CDRs; in which:
(1) any amino acid substitution is preferably a conservative amino acid substitution; and/or
(2) said amino acid sequence preferably only contains amino acid substitutions, and no amino acid deletions or insertions, compared to the above amino acid sequence(s); and/or chosen from the group consisting of amino acid sequences that have 3, 2 or only 1 (as indicated in the preceding paragraph) “amino acid difference(s)” with the mentioned CDR(s) of one of the above amino acid sequences, in which:
(1) any amino acid substitution is preferably a conservative amino acid substitution; and/or
(2) said amino acid sequence preferably only contains amino acid substitutions, and no amino acid deletions or insertions, compared to the above amino acid sequence(s).

In one embodiment, the anti-CDα 8 ISVD is BDSn:

Anti-CD8 BDSn Nb sequence (CDR1, CDR2, CDR3 underlined based on IMGT designation):

(SEQ ID NO: 419)
EVQLVESGGGLVQAGGSLRLSCAASGSTFSDYGVGWFRQAPGKGREFVAD
IDWNGEHTSYADSVKGRFATSRDNAKNTAYLQMNSLKPEDTAVYYCAADA
LPYTVRKYNYWGQGTQVTVSSGGCGGHHHHHH

In some embodiments, the anti-CD8α ISVD in a phospholipid-PEG-antibody conjugate is derived from SEQ ID NO: 419, such as listed in the Table C-4 below.

TABLE C-4
Anti-CD8 BDSn modified ISVDs.

| ISVD name | | |
|---|---|---|
| SES ID NO | Description | Sequence |
| A044300694 | CD8aBDSn (L11V, A14P, N53E, A68T, T69I,K8 | EVQLVESGGGVVQPGGSLRLSCAASGSTFSDYGVGWFR |
| SEQ ID NO: 10 | 3R,V89L, N101R, Q108L) | QAPGKGREFVADIDWEGEHTSYADSVKGRFTISRDNAK |
| | | NTAYLQMNSLRPEDTALYYCAADALPYTVRKYRYWGQG |
| | | TLVTVSS |
| A044300694 v1 | CD8aBDSn (L11V, A14P, N53E, A68T, T69I, K8 | EVQLVESGGGVVQPGGSLRLSCAASGSTFSDYGVGWER |
| SEQ ID NO: 11 | 3R, V89L, A94E, N101R, Q108L) | QAPGKGREFVADIDWEGEHTSYADSVKGRFTISRDNAK |
| | | NTAYLQMNSLRPEDTALYYCAEDALPYTVRKYRYWGQG |
| | | TLVTVSS |
| A044300694 v2 | CD8aBDSn (L11V, A14P, N53E, A68T, T69I, K8 | EVQLVESGGGVVQPGGSLRLSCAASGSTFSDYGVGWER |
| SEQ ID NO: 12 | 3R, V89L, A94Y, N101R, Q108L) | QAPGKGREFVADIDWEGEHTSYADSVKGRFTISRDNAK |
| | | NTAYLQMNSLRPEDTALYYCAYDALPYTVRKYRYWGQG |
| | | TLVTVSS |
| A044300694 v3 | CD8aBDSn (L11V,A14P, N53E, A68T, T69I, K8 | EVQLVESGGGVVQPGGSLRLSCAASGSTFSDYGVGWER |
| SEQ ID NO: 13 | 3R, V89L, V100aL, N101R, Q108L) | QAPGKGREFVADIDWEGEHTSYADSVKGRFTISRDNAK |
| | | NTAYLQMNSLRPEDTALYYCAADALPYTLRKYRYWGQG |
| | | TLVTVSS |
| A044300694 v4 | CD8aBDSn (L11V, A14P, N53E, A68T, T69I, K8 | EVQLVESGGGVVQPGGSLRLSCAASGSTFSDYGVGWER |
| SEQ ID NO: 14 | 3R, V89L, A94E, V100aL, N101R, Q108L) | QAPGKGREFVADIDWEGEHTSYADSVKGRFTISRDNAK |
| | | NTAYLQMNSLRPEDTALYYCAEDALPYTLRKYRYWGQG |
| | | TLVTVSS |
| A044300694 v5 | CD8aBDSn (L11V, A14P, N53E, A68T, T69I, K8 | EVQLVESGGGVVQPGGSLRLSCAASGSTFSDYGVGWER |
| SEQ ID NO: 15 | 3R, V89L, A94Y, V100aL, N101R, Q108L) | QAPGKGREFVADIDWEGEHTSYADSVKGRFTISRDNAK |
| | | NTAYLQMNSLRPEDTALYYCAYDALPYTLRKYRYWGQG |
| | | TLVTVSS |
| A044300694 v6 | CD8aBDSn (L11V, A14P, N53E, A68T, T691, Y7 | EVQLVESGGGVVQPGGSLRLSCAASGSTFSDYGVGWFR |
| SEQ ID NO: 16 | 9D, K83R, V89L, N101R, Q108L) | QAPGKGREFVADIDWEGEHTSYADSVKGRFTISRDNAK |
| | | NTADLQMNSLRPEDTALYYCAADALPYTVRKYRYWGQG |
| | | TLVTVSS |
| A044300694 v7 | CD8aBDSn (L11V, A14P, N53E, A68T, T691, Y7 | EVQLVESGGGVVQPGGSLRLSCAASGSTFSDYGVGWER |
| SEQ ID NO: 17 | 9D, K83R, V89L, V100aL, N101R, Q108L) | QAPGKGREFVADIDWEGEHTSYADSVKGRFTISRDNAK |
| | | NTADLQMNSLRPEDTALYYCAADALPYTLRKYRYWGQG |
| | | TLVTVSS |
| A044300694 v8 | CD8aBDSn (L11V, A14P, N53E, A68T, T691,Y7 | EVQLVESGGGVVQPGGSLRLSCAASGSTFSDYGVGWER |
| SEQ ID NO: 18 | 9D, K83R, V89L, A94E, V100aL, N101R, Q108L) | QAPGKGREFVADIDWEGEHTSYADSVKGRFTISRDNAK |
| | | NTADLQMNSLRPEDTALYYCAEDALPYTLRKYRYWGQG |
| | | TLVTVSS |
| A044300694 v9 | CD8aBDSn (L11V, A14P, N53E, A68T, T691, Y7 | EVQLVESGGGVVQPGGSLRLSCAASGSTFSDYGVGWER |
| SEQ ID NO: 19 | 9D, K83R, V89L, A94Y, V100aL, N101R, Q108L) | QAPGKGREFVADIDWEGEHTSYADSVKGRFTISRDNAK |
| | | NTADLQMNSLRPEDTALYYCAYDALPYTLRKYRYWGQG |
| | | TLVTVSS |
| A044300694 v10 | CD8aBDSn (L11V, A14P, N53E, A68T, T691, Y7 | EVQLVESGGGVVQPGGSLRLSCAASGSTFSDYGVGWER |
| SEQ ID NO: 20 | 9D, K83R, V89L, A94E, N101R, Q108L) | QAPGKGREFVADIDWEGEHTSYADSVKGRFTISRDNAK |
| | | NTADLQMNSLRPEDTALYYCAEDALPYTVRKYRYWGQG |
| | | TLVTVSS |
| A044300694 v11 | CD8aBDSn (L11V, A14P, N53E, A68T, T691, Y7 | EVQLVESGGGVVQPGGSLRLSCAASGSTFSDYGVGWER |
| SEQ ID NO: 21 | 9D, K83R, V89L, A94Y, N101R, Q108L) | QAPGKGREFVADIDWEGEHTSYADSVKGRFTISRDNAK |
| | | NTADLQMNSLRPEDTALYYCAYDALPYTVRKYRYWGQG |
| | | TLVTVSS |
| A044300694 v12 | CD8aBDSn (L11V, A14P, N53E, A68T, T691, Y7 | EVQLVESGGGVVQPGGSLRLSCAASGSTFSDYGVGWER |
| SEQ ID NO: 22 | 9S, K83R, V89L, V100aL, N101R, Q108L) | QAPGKGREFVADIDWEGEHTSYADSVKGRFTISRDNAK |
| | | NTASLQMNSLRPEDTALYYCAADALPYTLRKYRYWGQG |
| | | TLVTVSS |
| A044300694 v13 | CD8aBDSn (L11V, A14P, N53E, A68T, T691, Y7 | EVQLVESGGGVVQPGGSLRLSCAASGSTFSDYGVGWER |
| SEQ ID NO: 23 | 9S, K83R, V89L, A94E, V100aL, N101R, Q108L) | QAPGKGREFVADIDWEGEHTSYADSVKGRFTISRDNAK |
| | | NTASLQMNSLRPEDTALYYCAEDALPYTLRKYRYWGQG |
| | | TLVTVSS |
| A044300694 v14 | CD8aBDSn (L11V, A14P, N53E, A68T, T69I, Y7 | EVQLVESGGGVVQPGGSLRLSCAASGSTFSDYGVGWER |
| SEQ ID NO: 24 | 9S, K83R, V89L, A94Y, V100aL, N101R, Q108L) | QAPGKGREFVADIDWEGEHTSYADSVKGRFTISRDNAK |
| | | NTASLQMNSLRPEDTALYYCAYDALPYTLRKYRYWGQG |
| | | TLVTVSS |
| A044300694 v15 | CD8aBDSn (L11V, A14P, N53E, A68T, T691, Y7 | EVQLVESGGGVVQPGGSLRLSCAASGSTFSDYGVGWER |
| SEQ ID NO: 25 | 9S, K83R, V89L, N101R, Q108L) | QAPGKGREFVADIDWEGEHTSYADSVKGRFTISRDNAK |
| | | NTASLQMNSLRPEDTALYYCAADALPYTVRKYRYWGQG |
| | | TLVTVSS |
| A044300694 v16 | CD8aBDSn (L11V, A14P, N53E, A68T, T691, Y7 | EVQLVESGGGVVQPGGSLRLSCAASGSTFSDYGVGWFR |
| SEQ ID NO: 26 | 9S, K83R, V89L, A94E, N101R, Q108L) | QAPGKGREFVADIDWEGEHTSYADSVKGRFTISRDNAK |
| | | NTASLQMNSLRPEDTALYYCAEDALPYTVRKYRYWGQG |
| | | TLVTVSS |
| A044300694 v17 | CD8aBDSn (L11V, A14P, N53E, A68T, T691, Y7 | EVQLVESGGGVVQPGGSLRLSCAASGSTFSDYGVGWER |
| SEQ ID NO: 27 | 9S, K83R, V89L, A94Y, N101R, Q108L) | QAPGKGREFVADIDWEGEHTSYADSVKGRFTISRDNAK |
| | | NTASLQMNSLRPEDTALYYCAYDALPYTVRKYRYWGQG |
| | | TLVTVSS |

In some embodiments, the anti-CD8α ISVD in a phospholipid-PEG-antibody conjugate is T0347015C01, A044300805, A044300805_v1, A044300805_v2, A044300805_v3, A044300805_v4, A044300805_v5, A044300805_v6, A044300805_v7, or A044300805_v8 (e.g., SEQ ID NOs: 160 to 169), or a modified version wherein a polypeptide comprising cysteine (e.g., GGC) is added to the C-terminus of each (e.g., SEQ ID NOs: 28-36 and 44). In some embodiments, the anti-CD8α ISVD in the phospholipid-PEG-antibody conjugate is SEQ ID NO: 44.

In some embodiments, an anti-CD8α ISVD of the present disclosure binds to CD8 with an dissociation constant (KD) of 10⁻⁵ to 10⁻¹² moles/liter (M) or less, and preferably 10⁻⁷ to 10-12 moles/liter (M) or less and more preferably 10⁻⁸ to 10⁻¹² moles/liter (M), and/or with an association constant (KA) of at least 107 M⁻¹, preferably at least 10⁸ M⁻¹, more preferably at least 10⁹ M⁻¹, such as at least 10¹² M⁻¹; and in particular with a KD less than 500 nM, preferably less than 200 nM, more preferably less than 10 nM, such as less than 500 μM. The KD and KA values of the ISVD of the disclosure against CD8 can be determined in a manner known per se, for example using the assay described herein. More generally, the ISVDs described herein preferably have a dissociation constant with respect to CD8 that is as described in this paragraph.

Generally, it should be noted that the term ISVD as used herein in its broadest sense is not limited to a specific biological source or to a specific method of preparation. For example, as will be discussed in more detail below, the ISVD can be obtained (1) by isolating the VHH domain of a naturally occurring heavy chain antibody; (2) by expression of a nucleotide sequence encoding a naturally occurring VHH domain; (3) by “humanization” (as described below) of a naturally occurring VHH domain or by expression of a nucleic acid encoding such a humanized VHH domain; (4) by “camelization” (as described below) of a naturally occurring VH domain from any animal species, in particular a species of mammal, such as from a human being, or by expression of a nucleic acid encoding such a camelized VH domain; (5) by “camelisation” of a “domain antibody” or “Dab” as described by Ward et al (supra), or by expression of a nucleic acid encoding such a camelized VH domain; (6) using synthetic or semi-synthetic techniques for preparing proteins, polypeptides or other amino acid sequences; (7) by preparing a nucleic acid encoding a VHH domain, VH domain and/or dAb using techniques for nucleic acid synthesis, followed by expression of the nucleic acid thus obtained; and/or (8) by any combination of the foregoing. Suitable methods and techniques for performing the foregoing will be clear to the skilled person based on the disclosure herein and for example include the methods and techniques described in more detail hereinbelow.

In some embodiments, the anti-CD8α ISVDs of the present disclosure do not have an amino acid sequence that is exactly the same as (i.e. as a degree of sequence identity of 100% with) the amino acid sequence of a naturally occurring VH domain, such as the amino acid sequence of a naturally occurring VH domain from a mammal, and in particular from a human being.

One class of anti-CD8α ISVDs of the disclosure comprises ISVDs with an amino acid sequence that corresponds to the amino acid sequence of a naturally occurring VHH domain, but that has been “humanized”, i.e. by replacing one or more amino acid residues in the amino acid sequence of said naturally occurring VHH sequence by one or more of the amino acid residues that occur at the corresponding position(s) in a VH domain from a conventional 4-chain antibody from a human being (e.g., indicated above). It should be noted that such humanized anti-CD8α ISVDs of the present disclosure can be obtained in any suitable manner known per se (i.e. as indicated under points (1)-(8) above) and thus are not strictly limited to polypeptides that have been obtained using a polypeptide that comprises a naturally occurring VHH domain as a starting material.

Another class of anti-CD8α ISVDs of the present disclosure comprises Nanobody® ISVD with an amino acid sequence that corresponds to the amino acid sequence of a naturally occurring VH domain that has been “camelized”, i.e. by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. This can be performed in a manner known per se, which will be clear to the skilled person, for example on the basis of the further description below. Reference is also made to WO 94/04678. Such camelization may preferentially occur at amino acid positions which are present at the VH-VL interface and at the so-called Camelidae hallmark residues (see for example also WO 94/04678), as also mentioned below. In some embodiments, the VH domain or sequence that is used as a starting material or starting point for generating or designing the camelized ISVD is a VH sequence from a mammal, e.g., VH sequence of a human being. It should be noted that such camelized ISVD of the present disclosure can be obtained in any suitable manner known per se and thus are not strictly limited to polypeptides that have been obtained using a polypeptide that comprises a naturally occurring VH domain as a starting material.

For example, both “humanization” and “camelization” can be performed by providing a nucleotide sequence that encodes such a naturally occurring VHH domain or VH domain, respectively, and then changing, in a manner known per se, one or more codons in said nucleotide sequence such that the new nucleotide sequence encodes a humanized or camelized ISVD of the present disclosure, respectively, and then expressing the nucleotide sequence thus obtained in a manner known per se so as to provide the desired ISVD. Alternatively, based on the amino acid sequence of a naturally occurring VHH domain or VH domain, respectively, the amino acid sequence of the desired humanized or camelized ISVD of the present disclosure, respectively, can be designed and then synthesized de novo using techniques for peptide synthesis known per se. Also, based on the amino acid sequence or nucleotide sequence of a naturally occurring VHH domain or VH domain, respectively, a nucleotide sequence encoding the desired humanized or camelized ISVD can be designed and then synthesized de novo using techniques for nucleic acid synthesis known per se, after which the nucleotide sequence thus obtained can be expressed in a manner known per se so as to provide the desired ISVD.

Other suitable ways and techniques for obtaining ISVDs and/or nucleotide sequences and/or nucleic acids encoding the same, starting from (the amino acid sequence of) naturally occurring VH domains or preferably VHH domains and/or from nucleotide sequences and/or nucleic acid sequences encoding the same will be clear from the skilled person, and may for example comprising combining one or more amino acid sequences and/or nucleotide sequences from naturally occurring VH domains (such as one or more FR's and/or CDR's) with one or more one or more amino acid sequences and/or nucleotide sequences from naturally occurring VHH domains (such an one or more FR's or CDR's), in a suitable manner so as to provide (a nucleotide sequence or nucleic acid encoding) an ISVD. Also provided are compounds and constructs, and in particular proteins and polypeptides that comprise or essentially consists of at least one such amino acid sequence and/or ISVD of the disclosure (or suitable fragments thereof), and optionally further comprises one or more other groups, residues, moieties or binding units. In some embodiments, such further groups, residues, moieties, binding units or amino acid sequences may or may not provide further functionality to the amino acid sequence and/or ISVD (and/or to the compound or construct in which it is present) and may or may not modify the properties of the amino acid sequence and/or ISVD.

The disclosure also encompasses any polypeptide of the present disclosure that has been glycosylated at one or more amino acid positions, usually depending on the host used to express the polypeptide. A polypeptide can comprise an amino acid sequence of an anti-CD8α ISVD of the present disclosure, which is fused at its amino terminal end, at its carboxy terminal end, or both at its amino terminal end and at its carboxy terminal end with at least one further amino acid sequence. Such further amino acid sequence may comprise at least one further ISVD, so as to provide a polypeptide that comprises at least two, such as three, four or five, ISVDs, in which said ISVDs may optionally be linked via one or more linker sequences (as defined herein). Polypeptides of comprising the anti-CD8α ISVD of the present disclosure and one or more other ISVDs are multivalent polypeptides. In a multivalent polypeptide, the two or more ISVDs may be the same or different. For example, the two or more ISVDs in a multivalent polypeptide:
may be directed against the same antigen, i.e. against the same parts or epitopes of said antigen or against two or more different parts or epitopes of said antigen; and/or:
may be directed against the different antigens;
or a combination thereof.

Thus, a bivalent polypeptide, for example:
may comprise two identical ISVDs;
may comprise a first ISVD directed against a first part or epitope of an antigen and a second ISVD directed against the same part or epitope of said antigen or against another part or epitope of said antigen;
or may comprise a first ISVD directed against a first antigen and a second ISVD directed against a second antigen different from said first antigen;
whereas a trivalent polypeptide of the present disclosure for example:
may comprise three identical or different ISVDs directed against the same or different parts or epitopes of the same antigen;
may comprise two identical or different ISVDs directed against the same or different parts or epitopes on a first antigen and a third ISVD directed against a second antigen different from said first antigen; or
may comprise a first ISVD directed against a first antigen, a second ISVD directed against a second antigen different from said first antigen, and a third ISVD directed against a third antigen different from said first and second antigen.

The anti-CD8α ISVDs and polypeptides as disclosed herein can also be introduced and expressed in one or more cells, tissues or organs of a multicellular organism, for example for prophylactic and/or therapeutic purposes (e.g., as a gene therapy). For this purpose, the nucleotide sequences encoding the anti-CD8α ISVDs or polypeptides as disclosed herein can be introduced into the cells or tissues in any suitable way, for example as such (e.g., using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patient by administering a nucleic acid of the present disclosure or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the present disclosure and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y.). Giordano, Nature F Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. Nos. 5,580,859; 5,589,546; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

The disclosure also encompasses compositions comprising at least one ISVD or at least one polypeptide or construct of the present technology, at least one nucleic acid molecule encoding a polypeptide of the present technology or at least one vector comprising such a nucleic acid molecule. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

Accordingly, nucleic acid sequences encoding the anti-CD8α ISVDs as described herein, and expression construct and host cells comprising the nucleic acid sequence are also provided. Suitable host cells or host organisms are clear to the skilled person, and are for example any suitable fungal, prokaryotic or eukaryotic cell or cell line or any suitable fungal, prokaryotic or eukaryotic organism. Specific examples include HEK293 cells, CHO cells, Escherichia coli or Pichia pastoris. In one embodiment, the host is Pichia pastoris.

The present technology also provides a method for producing the ISVD and/or polypeptide of the present technology. The method may comprise transforming/transfecting a (non-human) host cell or host organism with a nucleic acid encoding the ISVD and/or polypeptide, expressing the ISVD and/or polypeptide in the host, optionally followed by one or more isolation and/or purification steps. In an embodiment, the method may comprise:
a) expressing, in a suitable (non-human) host cell or host organism or in another suitable expression system, a nucleic acid sequence or genetic construct encoding the ISVD and/or polypeptide; optionally followed by:
b) isolating and/or purifying the ISVD and/or polypeptide.

In another embodiment, the method may comprise:
a) cultivating and/or maintaining a (non-human) host or host cell that is capable of expressing the ISVD and/or polypeptide of the present technology and/or that comprises a nucleic acid or a genetic construct encoding the ISVD and/or polypeptide of the present technology, under suitable circumstances that are such that said (non-human) host or host cell is expresses the ISVD and/or polypeptide of the present technology;
optionally followed by:
b) isolating and/or purifying the ISVD and/or polypeptide produced.

Suitable (non-human) host cells or host organisms for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Pichia pastoris. In one embodiment, the host is Pichia pastoris.

Also disclosed are methods of using anti-CD8α ISVDs and polypeptides of the present disclosure.

In some embodiments, a polypeptide comprising an anti-CD8α ISVD can be used in the lipid nanoparticles of the present disclosure for delivering a nucleic acid into an immune cell, as described herein. In some embodiments, anti-CD8α ISVDs and polypeptides of the present disclosure can be used to treat a condition or a disease in a subject in need thereof. In some embodiments, such conditions or diseases include, but are not limited to, cancer, infections, immune disorders, autoimmune diseases.

Also provided is the ISVD of the present disclosure, the polypeptide or construct comprising the ISVD, the nucleic acid molecule or vector as described, or the composition comprising the ISVD, polypeptide, construct, nucleic acid molecule or vector of the present disclosure, for use in the diagnosis, prevention and/or treatment of at least one disease and/or disorder that is associated with CD8α, with its biological or pharmacological activity, and/or with the biological pathways or signaling in which CD8α is involved.

Also provided is the ISVD of the present disclosure, the polypeptide or construct comprising the ISVD, the nucleic acid molecule or vector as described, or the composition comprising the ISVD, polypeptide, construct, nucleic acid molecule or vector of the present disclosure for use in the diagnosis, prevention and/or treatment of a proliferative disease (such as cancer), an inflammatory disease, and/or an infectious disease. Therefore, also provided is the ISVD of the present disclosure, the polypeptide or construct of the present disclosure, the nucleic acid molecule or vector as described, or the composition comprising the ISVD, polypeptide, construct, nucleic acid molecule or vector of the present disclosure for use in the diagnosis, prevention and/or treatment of a proliferative disease (such as cancer), an inflammatory disease, and/or an infectious disease.

In some embodiments, a polypeptide comprising an anti-CD8α ISVD can be used in an imaging agent. In some embodiments, the imaging agent allows for the detection of human CD8, which is a specific biomarker found on the surface of a subset of T cells for diagnostic imaging of the immune system. Imaging of CD8 allows for the in vivo detection of T cell localization. Changes in T cell localization can reflect the progression of an immune response and can occur over time as a result of various therapeutic treatments or even disease states. In some embodiments, it is used for imaging T cell localization for immunotherapy.

In addition, CD8 plays a role in activating downstream signaling pathways that are important for the activation of cytolytic T cells that function to clear viral pathogens and provide immunity to tumors. CD8 positive T cells can recognize short peptides presented within the MHCI protein of antigen presenting cells. In some embodiments, a polypeptide comprising an anti-CD8α ISVD can potentiate signaling through the T cell receptor and enhance the ability of a subject to clear viral pathogens and respond to tumor antigens. Thus, in some embodiments, the antigen binding constructs provided herein can be agonists and can activate the CD8 target.

General methods for purifying ISVD polypeptides (such as VHs and VHHs) can be used to produce ISVD that is used to make phospholipid-PEG-ISVD (e.g., VHH) conjugate, such as those described in WO 2010/125187 and WO 2012/056000.

### III. Lipid Nanoparticles (LNPs) and Compositions

In one aspect, provided is a lipid nanoparticle (LNP) or LNP composition. In some embodiments, the LNP or LNP composition comprises (i) a lipid-immune cell targeting group conjugate.

In some embodiments, the LNPs also comprise (ii) an ionizable cationic lipid.

In some embodiments, the LNPs further comprise (iii) a structural lipid.

In some embodiments, the LNP further comprise (iv) a neutral phospholipid.

In some embodiments, the LNPs further comprise (v) a free PEG-lipid.

In some embodiments, the LNPs further comprise (vi) a payload.

In some embodiments, the LNPs comprises (i) to (vi) and any combination thereof.

In some embodiments, the LNPs comprise a formulation as illustrated in FIG. 50.

In some embodiments, the LNP or LNP composition comprises DSPE-PEG 3.4K-anti-CD8α ISVD conjugate (lipid-immune cell targeting group conjugate), Lipid 15 (ionizable cationic lipid), mRNA (payload), cholesterol (structural lipid), DSPC (neutral phospholipid), and PEG 2000-DPG (same as DPG-PEG 2K; free PEG-lipid). In some embodiments, the mRNA encodes a chimeric antigen receptor (CAR) comprises an anti-CD19 antibody, such as an anti-CD19 single domain antibody (sdAb or VHH). Non-limiting examples of each element are described herein.

In some embodiments, the LNP or LNP composition further comprises one or more additional components. For example, it may further comprise one or more additional components that are included in the LNP due to a manufacturing process that is used to produce the LNP. For example, as described herein, to produce a phospholipid-PEG-antibody conjugate, a phospholipid-PEG that does not have a bioconjugation linker may be included with the LNP or the LNP composition. In some embodiments, such phospholipid-PEG not having a bioconjugation linker is DSPE-PEG. In some embodiments, the DSPE-PEG has a PEG with a molecular weight smaller than the PEG in the phospholipid-PEG-antibody conjugate. In some embodiments, the DSPE-PEG has a PEG with a molecular weight of about 2.0 kDa, and the PEG in the phospholipid-PEG-antibody conjugate has a molecular weight of about 3.4 kDa.

In some embodiments, the DSPE-PEG2.0k has a concentration of less than about 0.1 mol %, 0.09 mol %, 0.08 mol %, 0.07 mol %, 0.06 mol %, 0.05 mol %, 0.04 mol %, 0.03 mol %, 0.02 mol %, 0.01 mol %, 0.009 mol %, 0.008 mol %, 0.007 mol %, 0.006 mol %, 0.005 mol %, 0.004 mol %, 0.003 mol %, 0.002 mol %, or 0.001 mol % in the LNP, or a composition comprising the LNP, excluding solvent.

In some embodiments, the DSPE-PEG2.0k has a concentration of less than about 0.1 mol %, 0.09 mol %, 0.08 mol %, 0.07 mol %, 0.06 mol %, 0.05 mol %, 0.04 mol %, 0.03 mol %, 0.02 mol %, 0.01 mol %, 0.009 mol %, 0.008 mol %, 0.007 mol %, 0.006 mol %, 0.005 mol %, 0.004 mol %, 0.003 mol %, 0.002 mol %, or 0.001 mol % in the LNP, or a composition comprising the LNP, excluding solvent. In some embodiments, the DSPE-PEG2.0k has a concentration of between about 0.01 mol % and about 0.02 mol % or between about 0.04 mol % and about 0.08 mol %, in the LNP, or a composition comprising the LNP, excluding solvent.

### A. Lipid-Immune Cell Targeting Group Conjugates

In some embodiments, the lipid-immune cell targeting group conjugate comprises the compound of Formula (II): [Lipid]-[optional linker]-[antibody]. In some embodiments, the Lipid of Formula (II) is a phospholipid. In some embodiments, the optional linker of Formula (II) is PEG. In some embodiments, the lipid-immune cell targeting group conjugate comprises DSPE-PEG 3.4K-anti CD8 antibody conjugate. In some embodiments, the conjugate is produced from conjugating an anti-CD8α immunoglobulin single variable domain (ISVD, such as a VHH) such as those described in the “II. Immunoglobulin single variable domain” section above, and DSPE-PEG having a bioconjugation linker, such as maleimide. In some embodiments, the DSPE-PEG maleimide is DSPE-PEG3.4K-maleimide. In some embodiments, the anti-CD8α ISVD in the conjugate comprises any one of SEQ ID NOs: 160 to 169 or any one of SEQ ID NOs 28-36 and 44, such as SEQ ID NO: 44, or any one of SEQ ID NOs: 10 to 27.

In some embodiments, the antibody of Formula (II) comprises an ISVD that can bind to and/or are directed against CD8α (CD8alpha) and that comprise CDR sequences that are generally as further defined herein, to suitable fragments thereof, as well as to polypeptides that comprise or essentially consist of one or more of such ISVDs and/or suitable fragments. In some embodiments, the antibody of Formula (II) comprises an ISVD comprising a sequence selected from the group consisting of SEQ ID NOs: 160 to 179. In some embodiments, the antibody of Formula (II) comprises
I) ISVD that is directed against CD8α and that has at least 80%, preferably at least 85%, such as 90% or 95% or more sequence identity with an ISVD comprising a sequence selected from the group consisting of SEQ ID NOs: 160 to 179; and/or
II) ISVD that cross-blocks the binding of the amino acid sequence selected from the group consisting of SEQ ID NOs: 160 to 179 to CD8α and/or that compete with at least the ISVD selected from the group consisting of SEQ ID NOs: 160 to 179 for binding to CD8α.

Such ISVDs, as part of Formula (II), may be as further described herein (and may for example be VHHs, including humanized VHHs, VHs, including human VHs, camelized VHs and camelized human VHs); as well as polypeptides of the disclosure that comprise one or more of such amino acid sequences (which may be as further described herein), and particularly bispecific (or multispecific) polypeptides as described herein, and nucleic acid sequences that encode ISVDs and polypeptides. Such ISVDs and polypeptides do not include any naturally occurring ligands.

In some embodiments, the CD8α is derived from a mammalian animal, such as a human being. In some embodiments, the antibody of Formula (II) comprises an ISVD directed against CD8α, that comprises:
a) the amino acid sequence selected from the group consisting of SEQ ID NOs: 160 to 179;
b) amino acid sequences that have at least 80% amino acid identity with a sequence selected from the group consisting of SEQ ID NOs: 160 to 179, or
c) amino acid sequences that have 3, 2, or 1 amino acid difference with a sequence selected from the group consisting of SEQ ID NOs: 160 to 179;
or any suitable combination thereof.

In some embodiments, the antibody of Formula (II) comprises an ISVD against CD8a, which consist of 4 framework regions (FR1 to FR4 respectively) and 3 complementarity determining regions (CDR1 to CDR3 respectively). In some embodiments, in such an ISVD:
(I) CDR1 comprises or essentially consists of an amino acid sequence selected from the group consisting of SEQ ID NOs: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering;
or amino acid sequences that have at least 80%, at least 90%, at least 95%, at least 99% or more sequence identity with a sequence selected from the group consisting of SEQ ID NO: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering;
and/or amino acids sequences that have 2 or only 1 amino acid difference(s) with a sequence selected from the group consisting of SEQ ID NO: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering, in which
any amino acid substitution is a conservative amino acid substitution; and/or
said amino acid sequence only contains amino acid substitutions, and no amino acid deletions or insertions, compared to a sequence selected from the group consisting of SEQ ID NO: 181, 188, 195, 202, 209, 216, 223, 230, 237, and 244 according to Abm numbering, and SEQ ID NOs: 251, 258, 265, 272, 279, 286, 293, 300, 307, and 314 according to Kabat numbering.
(II) CDR2 comprises or essentially consists of an amino acid sequence comprising a sequence selected from the group consisting of SEQ ID NO: 183, 190, 197, 204, 211, 218, 225, 232, 239 and 246 according to Abm numbering, and SEQ ID NOs: 253, 260, 267, 274, 281, 288, 295, 302, 309 and 316 according to Kabat numbering,
or amino acid sequences that have at least 80%, at least 90%, at least 95%, at least 99% or more sequence identity with a sequence selected from the group consisting of SEQ ID NO: 183, 190, 197, 204, 211, 218, 225, 232, 239 and 246 according to Abm numbering, and SEQ ID NOs: 253, 260, 267, 274, 281, 288, 295, 302, 309 and 316 according to Kabat numbering in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 183, 190, 197, 204, 211, 218, 225, 232, 239 and 246 according to Abm numbering, and SEQ ID NOs: 253, 260, 267, 274, 281, 288, 295, 302, 309 and 316 according to Kabat numbering; and/or amino acids sequences that have 2 or only 1 amino acid difference(s) with a sequence selected from the group consisting of SEQ ID NO: 183, 190, 197, 204, 211, 218, 225, 232, 239 and 246 according to Abm numbering, and SEQ ID NOs: 253, 260, 267, 274, 281, 288, 295, 302, 309 and 316 according to Kabat numbering in which
any amino acid substitution is a conservative amino acid substitution; and/or
said amino acid sequence only contains amino acid substitutions, and no amino acid deletions or insertions, compared to a sequence selected from the group consisting of SEQ ID NO: 183, 190, 197, 204, 211, 218, 225, 232, 239 and 246 according to Abm numbering and SEQ ID NOs: 253, 260, 267, 274, 281, 288, 295, 302, 309 and 316 according to Kabat numbering.
(III) CDR3 comprises or essentially consists of an amino acid sequence comprising a sequence selected from the group consisting of SEQ ID NO: 185, 192, 199, 206, 213, 220, 227, 234, 241 and 248 according to Abm numbering, and SEQ ID NOs: 255, 262, 269, 276, 283, 290, 297, 304, 311 and 318 according to Kabat numbering,
or amino acid sequences that have at least 80%, at least 90%, at least 95%, at least 99% or more sequence identity with a sequence selected from the group consisting of SEQ ID NO: 185, 192, 199, 206, 213, 220, 227, 234, 241 and 248 according to Abm numbering and SEQ ID NOs: 255, 262, 269, 276, 283, 290, 297, 304, 311 and 318 according to Kabat numbering, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 185, 192, 199, 206, 213, 220, 227, 234, 241 and 248 according to Abm numbering and SEQ ID NOs: 255, 262, 269, 276, 283, 290, 297, 304, 311 and 318 according to Kabat numbering;
and/or from the group consisting of amino acids sequences that have 2 or only 1 amino acid difference(s) with a sequence selected from the group consisting of SEQ ID NO: 185, 192, 199, 206, 213, 220, 227, 234, 241 and 248 according to Abm numbering and SEQ ID NOs: 255, 262, 269, 276, 283, 290, 297, 304, 311 and 318 according to Kabat numbering, in which any amino acid substitution is a conservative amino acid substitution; and/or
said amino acid sequence only contains amino acid substitutions, and no amino acid deletions or insertions, compared to a sequence selected from the group consisting of SEQ ID NO: 185, 192, 199, 206, 213, 220, 227, 234, 241 and 248 according to Abm numbering and SEQ ID NOs: 4 255, 262, 269, 276, 283, 290, 297, 304, 311 and 318 according to Kabat numbering.

In one embodiment, the antibody of Formula (II) comprises ISVD that comprises a CDR1 that is the amino acid sequence of SEQ ID NO: 244, a CDR2 that is the amino acid sequence of SEQ ID NO: 246 and a CDR3 that is the amino acid sequence of SEQ ID NO: 248 (Abm numbering) or that comprises a CDR1 that is the amino acid sequence of SEQ ID NO: 314, a CDR2 that is the amino acid sequence of SEQ ID NO: 316 and a CDR3 that is the amino acid sequence of SEQ ID NO: 318 (Kabat numbering).

CD8 binding ISVDs as disclosed herein may comprise one, two or all three of the CDRs explicitly listed above. In some embodiments, the anti-CD8αISVD is selected from the ISVDs described in Table C-1, Table C-2, and Table C-3 above. In some embodiments, the anti-CD8αISVD is selected from the ISVDs that have at least 80%, at least 85%, at least 90%, at least 95%, at least 96%, at least 97%, at least 98%, or at least 99% sequence identity with the ISVDs described in Table C-1, Table C-2, and Table C-3 above. In some embodiments, the anti-CD8ISVD is selected from the ISVDs that have at least 80%, at least 85%, at least 90%, at least 95%, at least 96%, at least 97%, at least 98%, or at least 99% sequence identity with the ISVDs described in Table C-4 above.

In some embodiments, the lipid-immune cell targeting group conjugate is present in the LNP in a range of 0.001-0.5 mol percent, 0.001-0.1 mol %, 0.01-0.5 mol %, 0.05-0.5 mol %, 0.1-0.5 mol %, 0.1-0.3 mol %, 0.1-0.2 mol %, 0.2-0.3 mol %, of about 0.01 mol %, about 0.05 mol %, about 0.1 mol %, about 0.15 mol %, about 0.2 mol %, about 0.25 mol %, about 0.3 mol %, about 0.35 mol %, about 0.4 mol %, about 0.45 mol %, or about 0.5 mol %. In some embodiments, the lipid-immune cell targeting group conjugate is present in the LNP in a range of 0.01 to 0.03 mol percent. In some embodiment, the lipid-immune cell targeting group conjugate is present in the LNP in about 0.015 to about 0.016 mol percent.

In some embodiments, the lipid-immune cell targeting group conjugate is presented in the LNP at a density from about 0.4 to 11.4, about 1.9 to 9.5, about 3.8 to 7.6, about 4.6 to 6.5, about 5.3 to 6.1, about 3.8, or about 5.7 micromoles of conjugate per gram of mRNA in the LNP. In some embodiments, the lipid-immune cell targeting group conjugate comprises SEQ ID NO: 9, SEQ ID NO: 169, or SEQ ID NO: 44.

In one aspect, the present disclosure provides methods for producing the lipid-immune cell targeting group conjugate. In some embodiments, the lipid-immune cell targeting group conjugate comprises lipid-linker-antibody, such as a phospholipid-PEG-antibody. In some embodiments, the antibody is an ISVD. In some embodiments, the methods comprise (i) purifying the ISVD; and (ii) conjugating the ISVD to a phospholipid-PEG, such as a DSPE-PEG (e.g., DSPE-PEG3.4k-maileimide).

In some embodiments, non-limiting examples for the method of producing the conjugate is as illustrated in FIG. 51 and FIG. 52.

In some embodiments, after the production/expression of the polypeptide of an ISVD, the host cell proteins and other impurities can be removed from the culture medium by routine means. For example, the host cell proteins and other impurities can be removed by centrifugation or filtration. The solution obtained by removal of the host cell proteins and other impurities from the culture medium is also referred to as culture supernatant or clarified culture supernatant.

The ISVD mixture product can be purified from culture supernatant by methods described herein. In some embodiments, the methods include, but are not limited to chromatographic methods, including size exclusion chromatography (SEC), ion exchange chromatography (IEX), affinity chromatography (AC), hydrophobic interaction chromatography (HIC), mixed-mode chromatography (MMC). These methods can be performed alone or in combination with other purification methods, e.g., precipitation. Suitable combinations of purification methods for ISVDs and ISVD containing polypeptides can be chosen as needed.

Any or all chromatographic steps can be carried out by any suitable mechanical means. Chromatography may be carried out, for example, in a column. The column may be run with or without pressure and from top to bottom or bottom to top. The direction of the flow of fluid in the column may be reversed during the chromatography process. Chromatography may also be carried out using a batch process in which the solid media is separated from the liquid used to load, wash, and elute the sample by any suitable means, including gravity, centrifugation, or filtration.

Chromatography may also be carried out by contacting the sample with a filter that absorbs or retains some molecules in the sample more strongly than others. In the following description, the various embodiments are mostly described in the context of chromatography carried out in a column. It is understood, however, that use of a column is merely one of several chromatographic modalities that may be used, and the illustration using a column does not limit the application to column chromatography, as those skilled in the art may readily apply the teachings to other modalities as well, such as those using a batch process or filter.

Suitable supports may be any currently available or later developed materials having the characteristics necessary to practice the claimed method, and may be based on any synthetic, organic, or natural polymer. For example, commonly used support substances include organic materials such as cellulose, polystyrene, agarose, sepharose, polyacrylamide, polymethacrylate, dextran and starch, and inorganic materials, such as charcoal, silica (glass beads or sand) and ceramic materials. Suitable solid supports are disclosed, for example, in Zaborsky “Immobilized Enzymes” CRC Press, 1973, Table IV on pages 28-46.

General method conditions, solutions and/or buffers, as well as their concentration ranges for use in the different chromatographic processes can be determined based on standard handbooks on chromatography (see e.g. Günter Jagschies, Eva Lindskog (ed.) Biopharmaceutical Processing, Development, Design, and Implementation of Manufacturing Processes, 1st Ed. 2017, Elsevier).

The first step of an ISVD polypeptide purification process is often referred to as “the capture step”. The purpose of the capture step is to have a first reduction of process-related impurities (for example, but not limited to, host cell proteins (HCPs), color and DNA) and to capture the ISVD polypeptide product while maintaining a high recovery. In one embodiment, the capture step refers to the first purification step on protein A chromatography in bind and elute mode.

In one embodiment, the ISVD polypeptide containing preparations may be purified by Protein A chromatography. Staphylococcal Protein A (SpA) is a 42 kDa protein composed of five nearly homologous domains named as E, D, A, B and C in order from the N-terminus (Sjodhal Eur. J. Biochem. 78: 471-490 (1977); Uhlen et al. J. Biol. Chem. 259: 1695-1702 (1984)). These domains contain approximately 58 residues, each sharing about 65%-90% amino acid sequence identity. Binding studies between Protein A and antibodies have shown that while all five domains of SpA (E, D, A, B and C) bind to an IgG via its Fc region, domains D and E exhibit significant Fab binding (Ljungberg et al. Mol. Immunol. 30(14): 1279-1285 (1993); Roben et al. J. Immunol. 154: 6437-6445 (1995); Starovasnik et al. Protein Sei. 8: 1423-1431 (1999). The Z-domain, a functional analogue and energy-minimized version of the B domain (Nilsson et al. Protein Eng. 1:107-113 (1987)), was shown to have negligible binding to the antibody variable domain region (Cedergren et al. Protein Eng. 6(4): 441-448 (1993); Ljungberg et al. (1993) supra; Starovasnik et al. (1999) supra).

Until recently, commercially available Protein A stationary phases employed SpA (isolated from Staphylococcus aureus or expressed recombinantly) as their immobilized ligand. Using these columns, it has not been possible to use alkaline conditions for column regeneration and sanitation as is typically done with other modes of chromatography using non-proteinaceous ligands (Ghose et al. Biotechnology and Bioengineering Yol. 92 (6): 665-73 (2005)). A new resin (MabSELECT™ SuRe) has been developed to withstand stronger alkaline conditions (Ghose et al. (2005) supra). Using protein engineering techniques, a number of asparagine residues were replaced in the Z-domain of protein A and a new ligand was created as a tetramer of four identically modified Z-domains (Ghose et al. (2005) supra).

Accordingly, purification methods can be carried out using commercially available Protein A columns according to manufacturers' specification. For instance, MabSELECT™ columns or MabSELECT™ SuRe columns (GE Healthcare Products) can be used. MabSELECT™ is a commercially available resin containing recombinant SpA as its immobilized ligand. Other commercially available sources of Protein A column including, but not limited to, PROSEP-ATM (Millipore, U.K.), which consists of Protein A covalently coupled to controlled pore glass, can be usefully employed. Other useful Protein A formulations include Protein A Sepharose FAST FLOW™ (Amersham Biosciences, Piscataway, NJ), Amsphere™ A3 (JSR Life Sciences), and TOYOPEARL™ 650M Protein A (TosoHaas Co., Philadelphia, PA).

Protein purification by Protein A-based chromatography may be performed in a column containing an immobilized Protein A ligand (typically a column packed with modified support of methacrylate copolymer or agarose beads to which is affixed an adsorbent consisting of Protein A or functional derivatives thereof). The column is typically equilibrated with a buffer and a sample containing a mixture of proteins (the target protein, plus contaminating proteins) is loaded onto the column. As the mixture passes through the column, the target protein binds to the adsorbent (Protein A or derivative thereof) within the column, while some unbound impurities and contaminants flow through. Bound protein is then eluted from the column. In this process the target protein is bound to the column while impurities and contaminants flow through. Target protein is subsequently recovered from the eluate.

After host cell proteins are removed, a sample containing ISVD can be subjected to a process often referred to as “the polish step” which aims at purity improvement. For instance, a chromatography step (e.g., ion exchange chromatography) in bind and elute mode can be used to remove/reduce product related variants (e.g., but not limited to, High-molecular Weight (HMW) species, Low-Molecular Weight (LMW) species, and other charged variants) as well as some process related impurities (e.g., but not limited to, HCP, residual Protein A, DNA) still present after the capture step.

### Method for Purifying ISVD Monomers

Due to the free cysteine linker at the C-terminus of the ISVD molecules, ISVD harvested from host cells normally present as a mixture of both monomers and dimers (e.g., two ISVD molecules are bound together through S-S). For example, a typical fermentation process may lead to a mixture having over 60% ISVD dimers and less than 40% monomers. Accordingly, there is a need to isolate only ISVD monomers from the mixture before they are conjugated to the phospholipid-PEG.

Therefore, in one aspect, the present disclosure provides a method for preparing a composition comprising monomers of an ISVD with a cysteine containing linker at its C-terminal end from a mixture of monomers of the ISVD and dimers of the ISVD. In some embodiments, the method comprises (a) reducing ISVD dimers in the mixture to ISVD monomers with a first reducing agent. In some embodiments, the first reducing agent comprises TCEP (tris(2-carboxyethyl) phosphine). In some embodiments, the step (a) is conducted around 15 to 25° C., optionally around 20-22° C. In some embodiments, the first reducing agent comprises 20×TCEP. In some embodiments, the step (a) takes about 16 to 20 hours. Optionally, the mixture of monomers of the ISVD and dimers of the ISVD is subjected to a step of removing host cell proteins and DNA before being subjected to step (a). In some embodiments, Protein A chromatography is used to remove host cell proteins and DNA.

In some embodiments, the method further comprises (b) purifying the ISVD monomers obtained in step (a) to get a purified composition comprising the ISVD monomers. In some embodiments, the step (b) comprises using a chromatograph, such as an ion exchange chromatography (IEX).

In some embodiments, the method further comprises (c) reducing the purified composition obtained in step (b) with a second reducing agent. In some embodiments, the second reducing agent is as the same or different from the first reducing agent used in step (a). In some embodiments, the reducing agent also comprises TCEP. In some embodiments, the first reducing agent comprises 10×TCEP. In some embodiments, the step (c) is conducted around 15° C. to 25° C., optionally around 20-22° C. In some embodiments, the step (c) takes about 16 to 20 hours.

In some embodiments, the method further comprises (d) purifying the reduced composition obtained in step (c) to obtain a composition comprising monomers of the ISVD. In some embodiments, the step (d) comprises Ultrafiltration/Diafiltration (UF/DF). In some embodiments, the UF/DF membrane has a molecular weight cut-off of 10 kDa.

In some embodiments, at least 50%, at least 55%, at least 60%, at least 65%, at least 70%, at least 75%, at least 80%, at least 85%, at least 90%, at least 95% or more ISVD in the composition obtained from step (d) are in monomeric form.

### Method for Conjugating ISVD to Lipid or LNPs Containing Lipid

In one aspect, also provided is a method for preparation of a composition comprising a lipid-PEG-antibody conjugate (e.g., a lipid-PEG-ISVD conjugate, such as a phospholipid-PEG-ISVD conjugate). In some embodiments, the lipid-PEG-ISVD is a phospholipid-PEG-ISVD. In some embodiments, the method comprises the following steps: (a) mixing a first composition comprising monomers of an ISVD comprising a linker (e.g., a linker for click chemistry reaction, such as a cysteine linker (e.g., GGC)), with a second composition comprising a first phospholipid-PEG comprising a bioconjugation linker under conditions that the phospholipid-PEG and the ISVD monomer can form a conjugate through click chemistry; (b) adding a quenching agent to the mixture obtained in step (a) under conditions that the conjugation reaction is quenched, wherein a composition comprising the phospholipid-PEG ISVD conjugate is obtained. As used herein, the term “quenching agent” refers to a compound that is able to compete with at least one of the substrates of the conjugation reaction there to slow down or stop the reaction. For example, the quenching agent can be cysteine when the conjugation click chemistry is based on cysteine-assisted click chemistry. In some embodiments, the composition obtained from the method comprises micelles wherein the micelles comprise the phospholipid-PEG-ISVD.

In some embodiments, the click chemistry reaction takes place in the mixture in step (a) under 15° C. to 25° C., such as about 15° C., 16° C., 17° C., 18° C., 19° C., 20° C., 21° C., 22° C., 23° C., 24° C., or 25° C. In some embodiments, the click chemistry reaction takes place under 20-22° C.

The click chemistry reaction in step (a) can take as long as needed to ensure a complete reaction. In some embodiments, the reaction takes about 1 to about 3 hours. In some embodiments, the reaction takes about 2 hours.

Step (b) as described in the method is a quenching step in which excessive quenching agent (e.g., cysteine for a cysteine-based click chemistry reaction) is added into the reaction so that the conjugation reaction is slowed down or stopped. In some embodiments, molar ratio between the added quenching agent and the phospholipid-PEG-maleimide is about 5:1 to about 1:1, such as about 5.0:1, 4.9:1, 4.8:1, 4.7:1, 4.6:1, 4.5:1, 4.4:1, 4.3:1, 4.2:1, 4.1:1, 4.0:1, 3.9:1, 3.8:1, 3.7:1, 3.6:1, 3.5:1, 3.4:1, 3.3:1, 3.2:1, 3.1:1, 3.0:1, 2.9:1, 2.8:1, 2.7:1, 2.6:1, 2.5:1, 2.4:1, 2.3:1, 2.2:1, 2.1:1, 2.0:1, 1.9:1, 1.8:1, 1.7:1, 1.6:1, 1.5:1, 1.4:1, 1.3:1, 1.2:1, 1.1:1, or 1.0:1. In some embodiments, the quenching step take about 5 minutes to 60 minutes, such as about 5 minutes, 10 minutes, 15 minutes, 20 minutes, 25 minutes, 30 minutes, 35 minutes, 40 minutes, 45 minutes, 50 minutes, 55 minutes or 60 minutes. In some embodiments, the quenching step takes place in the mixture in step (a) under 15° C. to 25° C., such as about 15° C., 16° C., 17° C., 18° C., 19° C., 20° C., 21° C., 22° C., 23° C., 24° C., or 25° C. In some embodiments, the click chemistry reaction takes place under 20-22° C.

In some embodiments, at least 50%, at least 55%, at least 60%, at least 65%, at least 70%, at least 75%, at least 80%, at least 85%, at least 90%, at least 95%, at least 99% or more of the ISVDs in the first composition are in monomeric form. In some embodiments, the phospholipid in the first phospholipid-PEG is a derivative of phosphatidylethanolamine. In some embodiments, the phospholipid in the first phospholipid-PEG comprises stearic acid acyl chains. In some embodiments, phospholipid in the first phospholipid-PEG is 1,2-Distearoyl-sn-glycero-3-phosphoethanolamine (DSPE). The PEG in the first phospholipid-PEG can have a weight from about 1 kDa to about 10 kDa, such as about 1 kDa, 1.5 kDa, 2.0 kDa, 2.5 kDa, 3.0 kDa, 3.5 kDa, 4.0 kDa, 4.5 kDa, 5.0 kDa, 5.5 kDa, 6.0 kDa, 6.5 kDa, 7.0 kDa, 7.5 kDa, 8.0 kDa, 8.5 kDa, 9.0 kDa, 9.5 kDa, or 10 kDa. In some embodiments, the PEG in the first phospholipid-PEG has a weight of about 3.4 kDa, and the conjugate is a DSPE-PEG 3.4K-ISVD conjugate. In some embodiments, the ISVD in the DSPE-PEG 3.4K-ISVD conjugate is an anti-CD8a ISVD, such as those described herein.

In some embodiments, the phospholipid-PEG has a bioconjugation linker, so that under proper conditions the first phospholipid-PEG molecules and the ISVD monomers can form a conjugate through click chemistry reaction. In some embodiments, the bioconjugation linker in the phospholipid-PEG has a maleimide group (e.g., phospholipid-PEG-maleimide, such as DSPE-PEG 3.4K-maleimide).

In some embodiments, a mixture obtained from the method described herein that contains micelles comprising the phospholipid-PEG-ISVD is contacted with a composition comprising LNPs. In some embodiments, such contact leads to that molecules in the micelles diffuse from the micelles and insert into the LNPs. For example, the phospholipid-PEG-ISVD in the micelles can diffuse from the micelles and insert into the LNPs to form new LNPs that comprise the phospholipid-PEG-ISVD. Depending on the binding specificity of the ISVD, the resulted LNPs can target to a specific cell type or tissue that the ISVD can specifically bind to. In some embodiments, the LNPs before step (a) comprise elements selected from the group of (i) an ionizable cationic lipid, (ii) a structural lipid, (iii) a neutral phospholipid, (iv) a free PEG-lipid, (v) a payload, such as mRNA, and (vi) any combination thereof. In some embodiments, the resulted LNPs obtained from the method comprise (i) the ionizable cationic lipid, (ii) the structural lipid, (iii) the neutral phospholipid, (iv) the free PEG-lipid, (v) the payload, such as mRNA, and (vi) the phospholipid-PEG-ISVD conjugate. In some embodiments, the LNPs resulted from the method comprise Lipid 15 (ionizable cationic lipid), cholesterol (structural lipid), DSPC (neutral phospholipid), PEG 2000-DPG (same as DPG-PEG 2K; free PEG-lipid), mRNA encodes a chimeric antigen receptor (CAR) comprises an anti-CD19 antibody, such as an anti-CD19 single domain antibody (sdAb or VHH), and DSPE-PEG 3.4K-anti-CD8α ISVD conjugate (lipid-immune cell targeting group conjugate).

Optionally, the composition comprising the phospholipid-PEG that has a bioconjugation linker for the conjugation reaction (e.g., a click chemistry reaction), further contains a second phospholipid-PEG that does not react with the ISVD. For example, the second phospholipid-PEG does not have the bioconjugation linker (e.g., a phospholipid-PEG that is not reactive in the bioconjugation reaction). In some embodiments, the second phospholipid-PEG that does not have the bioconjugation linker stabilizes the composition. For example, in some embodiments, the second phospholipid-PEG that does not have the bioconjugation linker stabilizes the micelle structure of the composition formed by the first phospholipid-PEG having a bioconjugation linker. In some embodiments, the second phospholipid-PEG that does not have the bioconjugation linker is commercially available. In some embodiments, the second phospholipid-PEG that does not have the bioconjugation linker to be used has at least a GMP grade. In some embodiments, the micelles formed through the bioconjugation reaction comprises a conjugate formed by the process as described herein. In some embodiments, the second phospholipid-PEG that does not have the bioconjugation linker stabilizes the micelle structure of the composition formed by the first phospholipid-PEG having a bioconjugation linker and the ISVD, and the second phospholipid-PEG that does not have the bioconjugation linker. In some embodiments, the second phospholipid-PEG that does not have the bioconjugation linker can be the same phospholipid in the first phospholipid-PEG, and the PEG in the second phospholipid-PEG has a different or the same molecular weight as the first phospholipid-PEG that has the bioconjugation linker. In some other embodiments, the second phospholipid-PEG that does not have the bioconjugation linker has a different phospholipid as in the first phospholipid-PEG, and the PEG in the second phospholipid-PEG has a different or the same molecular weight as the first phospholipid-PEG that has the bioconjugation linker. In some embodiments, the PEG in the second phospholipid-PEG has a molecular weight from about 0.5 kDa to about 10 kDa, such as about 0.5 kDa, 1.0 kDa, 1.5 kDa, 2.0 kDa, 2.5 kDa, 3.0 kDa, 3.5 kDa, 4.0 kDa, 4.5 kDa, 5.0 kDa, 5.5 kDa, 6.0 kDa, 6.5 kDa, 7.0 kDa, 7.5 kDa, 8.0 kDa, 8.5 kDa, 9.0 kDa, 9.5 kDa or 10.0 Kda. In some embodiments, the second phospholipid-PEG that does not have the bioconjugation linker can be the same phospholipid in the first phospholipid-PEG, and the PEG in the second phospholipid-PEG has a smaller molecular weight compared to the first phospholipid-PEG that has the bioconjugation linker. In some embodiments, the PEG in the first phospholipid-PEG with a bioconjugation linker has a molecular weight of about 3.4 kDa, and the PEG in the second phospholipid-PEG without a bioconjugation linker has a molecular weight of about 2.0 kDa. In some embodiments, the second phospholipid-PEG is also DSPE-PEG but with a smaller PEG size (e.g., DSPE-PEG2.0k) compared to the first DSPE-PEG having a bioconjugation linker (e.g., DSPE-PEG3.4k-maleimide). In some embodiments, the first DSPE-PEG is DSPE-PEG3.4k-maleimide and the second phospholipid-PEG is DSPE-PEG2.0k-OMeH.

In some embodiments, the first phospholipid-PEG molecules comprising the bioconjugation linker and the second phospholipid-PEG that does not have the bioconjugation linker are mixed first to form micelles before being conjugated to the antibody (e.g., the ISVD). In some embodiments, the molar ratio of the first phospholipid-PEG molecules comprising the bioconjugation linker to the second phospholipid-PEG that does not have the bioconjugation linker before they are mixed to form the micelles, or in the formed micelles is about 1:3 to about 1:1, such as about 1:3, about 2:3, about 1:1. In some embodiments, the molar ratio of the phospholipid-PEG molecules comprising the bioconjugation linker to the second phospholipid-PEG that does not have the bioconjugation linker before they are mixed to form micelles, or the ratio of the two components in formed micelles is about 2:3.

In some embodiments, the formed micelles are conjugated to one or more ISVD monomers that have a linker through a clicking reaction, such as a thiol-maleimide clicking reaction (e.g., through the reaction between a cysteine tag on the ISVD and a maleimide tail on the phospholipid-PEG). In some embodiments, the molar ratio of the ISVD monomers, the phospholipid-PEG molecules comprising the bioconjugation linker, and the second phospholipid-PEG that does not have the bioconjugation linker before the clicking reaction or in the formed conjugate is about 1:1:4 or 1:2:3. In some embodiments, the micelles having the phospholipid-PEG-antibody conjugated to them are mixed with a composition comprising LNPs to form targeted LNPs having the phospholipid-PEG-antibody conjugate.

As a result of the method described herein, micelles comprising the phospholipid-PEG-antibody (e.g., ISVD) conjugate are obtained. In some embodiments, the micelles also comprise a second phospholipid-PEG. In some embodiments, the micelles comprise DSPE-PEG3.4K-antibodody (e.g., ISVD) conjugate. In some embodiments, the micelles comprise DSPE-PEG3.4K-ISVD conjugate and DSPE-PEG-3.4-maleimide not conjugated to the antibody (e.g., ISVD). In some embodiments, the micelles comprise DSPE-PEG3.4K-ISVD conjugate, DSPE-PEG-3.4-maleimide not conjugated to the antibody (e.g., ISVD), and/or a second phospholipid-PEG that does not have a bioconjugate linker, such as DSPE-PEG2k. In some embodiments, the DSPE-PEG3.4K-ISVD conjugate has a concentration about 0.084 g/g to 0.15 g/g mRNA in the LNP. In some embodiments, the DSPE-PEG3.4K-ISVD conjugate has a concentration of about 0.084 g/g mRNA in the LNP. In some embodiments, the DSPE-PEG3.4K-ISVD conjugate has a concentration of about 0.11 g/g mRNA in the LNP. In some embodiments, the DSPE-PEG3.4K-ISVD conjugate has a concentration about 0.05 g/g to 0.15 g/g mRNA in the LNP. In some embodiments, the DSPE-PEG3.4K-ISVD conjugate is DSPE-PEG3.4K-anti-CD8 antibody conjugate.

The obtained micelles comprising the phospholipid-PEG-antibody (e.g., ISVD) can be further used to produce targeted LNPs described in the present disclosure.

In another aspect, also provided is a method for conjugating an antibody (e.g., an ISVD) to a LNP comprising at least one lipid-PEG (e.g., a phospholipid-PEG). In some embodiments, the lipid-PEG-ISVD is a phospholipid-PEG-ISVD. In some embodiments, the method comprises the following steps: (a) mixing a first composition comprising monomers of an ISVD comprising a cysteine containing linker, with a second composition comprising a LNP that contains a first phospholipid-PEG, wherein the first phospholipid-PEG comprises a bioconjugation linker under conditions that the phospholipid-PEG and the ISVD monomer can form a conjugate through click chemistry; (b) adding a quenching agent to the mixture obtained in step (a) under conditions that the conjugation reaction is quenched, wherein a composition comprising LNPs comprising the phospholipid-PEG ISVD conjugate is obtained. In some embodiments, the LNPs that contain first phospholipid-PEG molecules also comprise other elements selected from the group of (i) an ionizable cationic lipid, (ii) a structural lipid, (iii) a neutral phospholipid, (iv) a free PEG-lipid, (v) a payload, such as mRNA, and (vi) any combination thereof. In some embodiments, the LNPs comprise Lipid 15 (ionizable cationic lipid), mRNA (payload), cholesterol (structural lipid), DSPC (neutral phospholipid), and PEG 2000-DPG (same as DPG-PEG 2K; free PEG-lipid). In some embodiments, the mRNA encodes a chimeric antigen receptor (CAR) comprises an anti-CD19 antibody, such as an anti-CD19 single domain antibody (sdAb or VHH). In some embodiments, the LNPs produced from a method as described herein further comprises DSPE-PEG 3.4K-anti-CD8α ISVD conjugate (lipid-immune cell targeting group conjugate).

In some embodiments, the antibody in the phospholipid-PEG-antibody is an immunoglobulin single variable domain. In some embodiments, the antibody in the phospholipid-PEG-antibody is selected from a VHH or sdAb (including humanized VHH or sdAb), a VH (including camelized VH, human VH, camelized human VH, dAb) and a VL. In some embodiments, the antibody in the phospholipid-PEG-antibody is an immunoglobulin single variable domain that comprises at least two disulfide bridges.

It is known that all VHHs contain at least one disulfide bridge, between the cysteine residue at position 22 and the cysteine residue at position 92 (numbering according to Kabat; Muyldermans and Lauwereys 1999, J. Mol. Recognit. 12: 131). Although most VHHs contain only this single disulfide bridge, it is also known that some VHHs contain a total of two (or in exceptional cases three) disulfide bridges. For example, a class of VHHs referred to as “VHH-1 type”, “VHH-1 class”, or the like (as further defined herein) commonly has a second disulfide bridge between the cysteine residue in CDR2 at position 50 and a cysteine residue present in CDR3 (or in exceptional cases in CDR1 or CDR2). Also, some VHHs derived from camels or dromedaries often have a disulfide bridge between a cysteine residue present in CDR1 (or at position 45 in FR2) and a cysteine residue present in CDR3 (Vu et al. 1997, Mol. Immunol. 34: 1121; Muyldermans and Lauwereys 1999). Some VHHs derived from llamas sometimes have a disulfide bridge between cysteine residues present in CDR1 (such as at position 33) and a cysteine residue present in CDR3 (Vu et al., 1997).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in one of the framework regions and a cysteine residue in one of the CDR regions.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in one of the framework regions and a cysteine residue in CDR3.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in framework two (FR2) and a cysteine residue in CDR3.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue at position 45 in framework two (FR2) and a cysteine residue in CDR3 (as in some VHHs derived from camels and dromedaries).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in one CDR and a cysteine residue in another CDR.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR3 and a cysteine residue in another CDR (in particular in CDR1, as in some VHHs derived from camels, dromedaries and llamas).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR1 and a cysteine residue in another CDR.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR1 and a cysteine residue in CDR1.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR1 and a cysteine residue in CDR3 (as in some VHHs derived from camels, dromedaries and llamas).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine at position 33 and a cysteine residue in CDR3 (as in some VHHs derived from camels, dromedaries and llamas).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR2 and a cysteine residue in another CDR.

In some embodiments, the immunoglobulin single variable domain comprises a disulfide bridge between the cysteine residue at position 22 and the cysteine residue at position 92, and further comprises a disulfide bridge that is formed between a cysteine residue in CDR2 and a cysteine residue in CDR2.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR2 and a cysteine residue in CDR3 (as in some VHHs derived from llamas).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue at position 50 and a cysteine residue in another CDR, such as CDR1, CDR2 or CDR3 (as in VHHs of the VHH1 type).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue at position 50 and a cysteine residue in CDR3 (as in VHHs of the VHH1 type).

In some embodiments, the immunoglobulin single variable domain in the phospholipid-PEG-antibody may be a “VHH1 type immunoglobulin single variable domain”. An amino acid such as e.g., an immunoglobulin single variable domain or polypeptide is said to be a “VHH1 type immunoglobulin single variable domain” or “VHH type 1 sequence”, if said VHH1 type immunoglobulin single variable domain or VHH type 1 sequence has 85% identity (using the blastp algorithm with standard setting, i.e. blosom62 scoring matrix) to the VHH1 consensus sequence (SEQ ID NO: 569): QVQLVESGGGLVQPGGSLRLSCAASGFTLDYYAIGWFRQAPGKEREGVSCISSSDGS TYYADSVKGRFTISRDNAKNTVYLQMNSLKPEDTAVYYCAA) and mandatorily has a cysteine in position 50, i.e. Cys50 (using Kabat numbering). These VHH1 type immunoglobulin single variable domains generally have (or are capable of forming) a disulfide bridge between Cys50 and a cysteine residue in CDR3 (or in exceptional cases CDR1 or CDR2).

In some embodiments, the immunoglobulin single variable domain in the phospholipid-PEG-antibody is another immunoglobulin single variable domains that comprise two or more disulfide bridges (such as (single) domain antibodies, dAbs, IgNAR domains from sharks, etc.). In some embodiments, the immunoglobulin single variable domain in the phospholipid-PEG-antibody comprises at least two disulfide bridges. In some embodiments, the immunoglobulin single variable domain in the phospholipid-PEG-antibody belongs to the group of VHH1 type immunoglobulin single variable domains.

In some embodiments, the immunoglobulin single variable domain in the phospholipid-PEG-antibody does not belong to the group of VHH1 type immunoglobulin single variable domains, but belongs to another group of immunoglobulin single variable domains, such as the VHH2, VHH3 or any other type of immunoglobulin single variable domains.

As such, in some embodiments, the lipid-immune cell targeting group conjugate comprises DSPE-PEG 3.4K-ISVD conjugate. In some embodiments, the lipid-immune cell targeting group conjugate comprises DSPE-PEG 3.4K-VHH conjugate. In some embodiments, the lipid-immune cell targeting group conjugate comprises DSPE-PEG 3.4K-anti-CD8α VHH conjugate. In some embodiments, the lipid-immune cell targeting group conjugate comprises DSPE-PEG 3.4K-VHH1 conjugate. In some embodiments, the lipid-immune cell targeting group conjugate comprises DSPE-PEG 3.4K-anti-CD8α VHH1 conjugate.

In some embodiments, the immunoglobulin single variable domain comprised in the phospholipid-PEG-antibody is an ISVD directed against CD8α, that comprises:
a) the amino acid sequence selected from the group consisting of SEQ ID NOs: 160 to 179;
b) amino acid sequences that have at least 80% amino acid identity with a sequence selected from the group consisting of SEQ ID NOs: 160 to 179, or
c) amino acid sequences that have 3, 2, or 1 amino acid difference with a sequence selected from the group consisting of SEQ ID NOs: 160 to 179;
or any suitable combination thereof.

In some embodiments, the immunoglobulin single variable domain comprised in the phospholipid-PEG-antibody an ISVD against CD8α, which consist of 4 framework regions (FR1 to FR4 respectively) and 3 complementarity determining regions (CDR1 to CDR3 respectively) in which:
(I) CDR1 comprises or essentially consists of an amino acid sequence selected from the group consisting of SEQ ID NOs: 181 and 188, according to Abm numbering, and SEQ ID NOs: 251, according to Kabat numbering;
or of amino acid sequences that have at least 80%, at least 90%, at least 95%, at least 99% or more sequence identity with a sequence selected from the group consisting of SEQ ID NO: 181 and 188, according to Abm numbering, and SEQ ID NOs: 251, according to Kabat numbering, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 181 and 188, according to Abm numbering, and SEQ ID NOs: 251, according to Kabat numbering; and/or amino acids sequences that have 2 or only 1 amino acid difference(s) with a sequence selected from the group consisting of SEQ ID NO: 181 and 188, according to Abm numbering, and SEQ ID NO: 251, according to Kabat numbering, in which any amino acid substitution is a conservative amino acid substitution; and/or said amino acid sequence only contains amino acid substitutions, and no amino acid deletions or insertions, compared to a sequence selected from the group consisting of SEQ ID NO: 181 and 188, according to Abm numbering, and SEQ ID NO: 251, according to Kabat numbering.
(II) CDR2 comprises or essentially consists of an amino acid sequence comprising a sequence selected from the group consisting of SEQ ID NO: 183 and 190, according to Abm numbering, and SEQ ID NOs: 253 and 260, according to Kabat numbering, or amino acid sequences that have at least 80%, at least 90%, at least 95%, at least 99% or more sequence identity with a sequence selected from the group consisting of SEQ ID NO: 183 and 190, according to Abm numbering, and SEQ ID NOs: 253 and 260, according to Kabat numbering in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 183 and 190, according to Abm numbering, and SEQ ID NOs: 253 and 260, according to Kabat numbering;
and/or amino acids sequences that have 2 or only 1 amino acid difference(s) with a sequence selected from the group consisting of SEQ ID NO: 183 and 190, according to Abm numbering, and SEQ ID NOs: 253 and 260, according to Kabat numbering, in which any amino acid substitution is a conservative amino acid substitution; and/or said amino acid sequence only contains amino acid substitutions, and no amino acid deletions or insertions, compared to a sequence selected from the group consisting of SEQ ID NO: 183 and 190, according to Abm numbering and SEQ ID NOs: 253 and 260, according to Kabat numbering.
(III) CDR3 comprises or essentially consists of an amino acid sequence comprising a sequence selected from the group consisting of SEQ ID NO: 185, 192, 199 and 206, according to Abm numbering, and SEQ ID NOs 185, 192, 199 and 206, according to Kabat numbering, or amino acid sequences that have at least 80%, at least 90%, at least 95%, at least 99% or more sequence identity with a sequence selected from the group consisting of SEQ ID NO: 185, 192, 199 and 206, according to Abm numbering and SEQ ID NOs: 185, 192, 199 and 206, according to Kabat numbering, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 185, 192, 199 and 206, according to Abm numbering and SEQ ID NOs: 185, 192, 199 and 206, according to Kabat numbering;
and/or amino acids sequences that have 2 or only 1 amino acid difference(s) with a sequence selected from the group consisting of SEQ ID NO: 185, 192, 199 and 206, according to Abm numbering and SEQ ID NOs: 185, 192, 199 and 206, according to Kabat numbering, in which any amino acid substitution is a conservative amino acid substitution; and/or said amino acid sequence only contains amino acid substitutions, and no amino acid deletions or insertions, compared to a sequence selected from the group consisting of SEQ ID NO: 185, 192, 199 and 206, according to Abm numbering and SEQ ID NOs: 185, 192, 199 and 206, according to Kabat numbering.

In one embodiment, the immunoglobulin single variable domain comprised in the phospholipid-PEG-antibody comprises a CDR1 that is the amino acid sequence of SEQ ID NO: 244, a CDR2 that is the amino acid sequence of SEQ ID NO: 246 and a CDR3 that is the amino acid sequence of SEQ ID NO: 248 (Abm numbering) or comprises a CDR1 that is the amino acid sequence of SEQ ID NO: 314, a CDR2 that is the amino acid sequence of SEQ ID NO: 316 and a CDR3 that is the amino acid sequence of SEQ ID NO: 318 (Kabat numbering).

CD8 binding ISVDs in the phospholipid-PEG-antibody may comprise one, two or all three of the CDRs explicitly listed above. In some embodiments, the anti-CD8αISVD in the phospholipid-PEG-antibody is selected from the ISVDs described in Table C-1, Table C-2, and Table C-3. In some embodiments, the anti-CD8αISVD in the phospholipid-PEG-antibody is selected from the ISVDs that have at least 80%, at least 85%, at least 90%, at least 95%, at least 96%, at least 97%, at least 98%, or at least 99% sequence identity with the ISVDs described in Table C-1, Table C-2, and Table C-3. In some embodiments, the anti-CD8ISVD in the phospholipid-PEG-antibody is selected from the ISVDs that have at least 80%, at least 85%, at least 90%, at least 95%, at least 96%, at least 97%, at least 98%, or at least 99% sequence identity with the ISVDs described in Table C-4.

In some embodiments, the anti-CD8α ISVD comprises or is an ISVD described herein, such as: a) the amino acid sequence selected from the group consisting of SEQ ID NOs: 160 to 179; b) amino acid sequences that have at least 80% amino acid identity with a sequence selected from the group consisting of SEQ ID NOs: 160 to 179; or c) amino acid sequences that have 3, 2, or 1 amino acid difference with a sequence selected from the group consisting of SEQ ID NOs: 160 to 179. In some embodiments, the anti-CD8αISVD comprises SEQ ID NP: 9, SEQ ID NO: 169, or SEQ ID NO: 179.

In some embodiments, the phospholipid in the phospholipid-PEG-antibody is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), ceramide. In some embodiments, the phospholipid is DSPE.

In some embodiments, the antibody is covalently coupled to the phospholipid in Formula (II) ([Lipid]-[optional linker]-[antibody]), phospholipid-linker-antibody, or phospholipid-PEG-antibody, wherein the linker comprises polyethylene glycol (PEG). In some embodiments, PEG has a molecular weight of about 1.0 kDa to about 6 kDa. In some embodiments, the PEG has a molecular weight about 3400 Da (e.g., PEG 3400 (PEG 3.4K)). In some embodiments, phospholipid-PEG molecule is conjugated to the antibody through a click chemistry reaction. In some embodiments, the antibody is an ISVD. The phospholipid-PEG can be conjugated to the ISVD according to methods described herein.

The lipid-PEG-antibody conjugate of the present disclosure can be inserted into an LNP to form a targeted LNP. In some embodiment, the lipid-PEG-antibody conjugate can help the LNP to target a specific cell, a specific tissue, or a specific organ. In some embodiments, the lipid-PEG-antibody conjugate comprises an immune cell targeting group. For example, the antibody in the conjugate can be an antibody that specifically binds to immune cells. In some embodiments, the lipid-immune cell targeting group conjugate in the LNP of the present disclosure comprises DSPE-PEG-anti-CD8α antibody. In some embodiments, the conjugate comprises DSPE-PEG3.4k-anti-CD8α antibody. In some embodiments, the conjugate comprises DSPE-PEG3.4k-A044300805_v8 (SEQ ID NO: 9, SEQ ID NO: 169 or SEQ ID NO: 179).

In some embodiments, the conjugate of the present disclosure (e.g., DSPE-PEG3.4k-A044300805_v8) can be present in the LNP in a range of 0.001-0.5 mol percent, 0.001-0.1 mol %, 0.01-0.5 mol %, 0.05-0.5 mol %, 0.1-0.5 mol %, 0.1-0.3 mol %, 0.1-0.2 mol %, 0.2-0.3 mol %, of about 0.01 mol %, about 0.05 mol %, about 0.1 mol %, about 0.15 mol %, about 0.2 mol %, about 0.25 mol %, about 0.3 mol %, about 0.35 mol %, about 0.4 mol %, about 0.45 mol %, or about 0.5 mol %. In some embodiments, the conjugate comprises DSPE-PEG3.4k-A044300805_v8, and is present in the LNP in a range of 0.01 to 0.02 mol percent. In some embodiment, the conjugate comprising DSPE-PEG3.4k-A044300805_v8 is present in the LNP is about 0.015 to about 0.016 mol percent.

An exemplary method for making a targeted LNP that comprises any of the ISVD described herein is shown in FIG. 53.

### B. Ionizable Cationic Lipids

Provided herein are ionizable cationic lipids that can be used to produce lipid nanoparticle compositions to facilitate the delivery of a payload (e.g., a nucleic acid, such as a DNA or RNA, such as an mRNA, such as any of SEQ ID NOs:570-575) disposed therein to cells, e.g., mammalian cells, e.g., immune cells. In some embodiments, an ionizable cationic lipid of the present disclosure is used to produce LNPs as described herein. The ionizable cationic lipids have been designed to enable intracellular delivery of a nucleic acid, e.g., mRNA, to the cytosolic compartment of a target cell type and rapidly degrade into non-toxic components. The complex functionalities of the ionizable cationic lipids are facilitated by the interplay between the chemistry and geometry of the ionizable lipid head group, the hydrophobic “acyl-tail” groups and the linkers connecting the head group and the acyl tail groups. Typically, the substituents of the ionizable amine head group are chosen to tune the apparent pKa of amine head group in the LNP formulation to fall in the range of 6-8, such as between 6.2-7.4, or between 6.7-7.2. As such, the amine head groups remains strongly cationic under acidic formulation conditions (e.g., pH 4-pH 5.5), neutral or slightly anionic or slightly cationic (typical zeta potential of 0±5 mV) in physiological pH (7.4), but strongly cationic in the early and late endosomal compartments (e.g., pH 5.5-pH 7). The acyl-tail groups play a key role in fusion of the lipid nanoparticle with endosomal membranes and membrane destabilization through structural perturbation. The three-dimensional structure of the acyl-tail (determined by its length, and degree and site of unsaturation) along with the relative sizes of the head group and tail group are thought to play a role in promoting membrane fusion, and hence lipid nanoparticle endosomal escape (a key requirement for cytosolic delivery of a nucleic acid payload). The linker connecting the head group and acyl tail groups is designed to degrade by physiologically prevalent enzymes (e.g., esterases, or proteases) or by acid catalyzed hydrolysis.

In one aspect, the present invention provides a compound represented by Formula

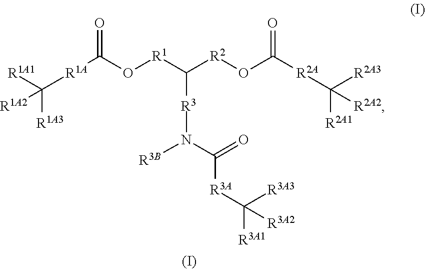

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is

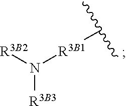

R^{3B1} is C₁₋₆ alkylene; and
R^{3B2} and R^{3B3} are each independently H, unsubstituted C₁₋₆ alkyl, or C₁-6 alkyl substituted with 1 or 2-OH.

In some embodiments, provided is a compound of Formula (I-1):

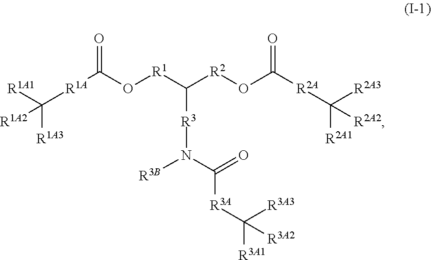

or a salt thereof, wherein:
R¹, R², and R³ are each independently a bond or methylene;
R^{1A} and R^{2A} are each C₁₋₁₀ alkylene;
R^{3A} is C₁₋₅ alkylene;
R^{1A1}, R^{1A2}, R^{2A1}, R^{2A2}, R^{3A1}, and R^{3A2} are each H;
R^{1A3} and R^{2A3} are each C₁₋₂₀ alkenyl;
R^{3A3} is —C(O)O(C₁₋₂₀ alkyl);
R^{3B} is

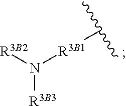

R^{3B1} is C₂₋₄ alkylene; and
R^{3B2} and R^{3B3} are each methyl.

In some embodiments, R^{3B1} is —(CH₂)₃—.

In some embodiments, the ionizable cationic lipid is

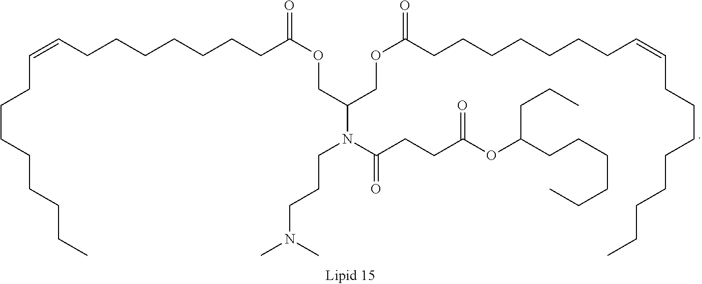 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

In one aspect, the present invention provides a compound of Formula (I-A):

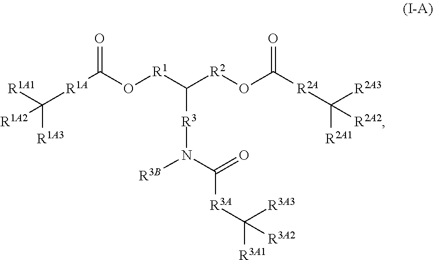

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is

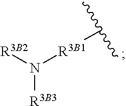

R^{3B1} is C₁₋₆ alkylene; and
R^{3B2} and R^{3B3} are each independently H, unsubstituted C₁₋₆ alkyl, or C₁-6 alkyl substituted with one or more substituents each independently selected from the group consisting of —OH and —O—(C₁₋₆ alkyl).

Any of the variables or substituents provided herein is unsubstituted or substituted with one or more substituents. In some embodiments, any of the variables or substituents provided herein is optionally substituted. In some embodiments, any of the variables or substituents provided herein is optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13}, and —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃-10 cycloalkyl, C₆₋₁₄ aryl, 5- to 10-membered heteroaryl, or 3- to 10-membered heterocyclyl, each of which is optionally substituted.

In some embodiments, R¹, R², and R³ are each independently a bond or C₁₋₃ alkylene. In some embodiments, R¹, R², and R³ are each independently a bond or methylene. In some embodiments, R¹ and R² are each methylene and R³ is a bond. In some embodiments, R¹, R², and R³ are each methylene. In some embodiments, R¹, R², and R³ are each independently unsubstituted or substituted. In some embodiments, R¹, R², and R³ are unsubstituted.

In some embodiments, R^{1A}, R^{2A}, and R^{3A} are each independently a bond or C₁₋₁₀ alkylene. In some embodiments, R^{1A}, R^{2A}, and R^{3A} are each independently a bond or —(CH₂)₁₋₁₀—. In some embodiments, R^{1A} and R^{2A} are each independently a bond, —CH₂—, —(CH₂)₂—, —(CH₂)₃—, —(CH₂)₄—, —(CH₂)₅—, —(CH₂)₆—, —(CH₂)₇—, or —(CH₂)₈—. In some embodiments, R^{1A} and R^{2A} are each a bond, each —CH₂—, each —(CH₂)₂—, each —(CH₂)₃—, each —(CH₂)₄—, each —(CH₂)₅—, each —(CH₂)₆—, each —(CH₂)₇—, or each —(CH₂)₈—. In some embodiments, R^{1A} and R^{2A} are each independently a bond, —(CH₂)₂—, —(CH₂)₄—, —(CH₂)₆—, —(CH₂)₇—, or —(CH₂)₈—. In some embodiments, R^{1A} and R^{2A} are each a bond, each —(CH₂)₂—, each —(CH₂)₄—, each —(CH₂)₆—, each —(CH₂)₇—, or each —(CH₂)₈—. In some embodiments, R^{3A} is a bond, —CH₂—, —(CH₂)₂—, or —(CH₂)₇—. In some embodiments, R^{1A}, R^{2A}, and R^{3A} are each independently unsubstituted or substituted. In some embodiments, R^{1A}, R^{2A}, and R^{3A} are unsubstituted.

In some embodiments, R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}. In some embodiments, R^{1A1}, R^{1A2}, R^{1A3}, R^{2A1}, R^{2A2}, R^{2A3}, R^{3A1}, R^{3A2}, and R^{3A3} are each independently H, C₁₋₁₅ alkyl, —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), —(CH₂)₀₋₄OC(O)CH(C₁₋₁₀ alkyl)(C₁₋₁₅ alkyl), —(CH₂)₀₋₄C(O)OCH₂(C₁₋₁₅ alkyl), or —(CH₂)₀₋₄OC(O)CH₂(C₁₋₁₅ alkyl). In some embodiments, R^{1A1}, R^{1A2}, R^{1A3}, R^{2A1}, R^{2A2}, R^{2A3}, R^{3A1}, R^{3A2}, R^{3A3}, R¹, R², R³, R^{1A}, R^{2A}, and R^{3A} are each independently unsubstituted or substituted. In some embodiments, R^{1A1}, R^{1A2}, R^{1A3}, R^{2A1} R^{2A2}, R^{2A3}, R^{3A1}, R^{3A2}, R^{3A3}, R¹, R², R³, R^{1A}, R^{2A}, and R^{3A} are each unsubstituted. In some embodiments, R^{1A1}, R^{1A2}, R^{1A3}, R^{2A1}, R^{2A2}, R^{2A3}, R^{3A1}, R^{3A2}, and R^{3A3} are each independently unsubstituted or substituted. In some embodiments, R^{1A1}, R^{1A2}, R^{1A3}, R^{2A1}, R^{2A2}, R^{2A3}, R^{3A1}, R^{3A2}, and R^{3A3} are each unsubstituted. In some embodiments, R¹, R², R³, R^{1A}, R^{2A}, and R^{3A} are each independently unsubstituted or substituted. In some embodiments, R¹, R², R³, R^{1A}, R^{2A}, and R^{3A} are each unsubstituted. In some embodiments, R¹, R², and R³ are each unsubstituted.

In some embodiments, R^{3B1} is unsubstituted. In some embodiments, R^{3B1} is not substituted with oxo.

In some embodiments, R^{1A1} and R^{2A1} are each independently —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H. In some embodiments, R^{1A1} and R^{2A1} are each —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H. In some embodiments, R^{1A1} and R^{2A1} are each

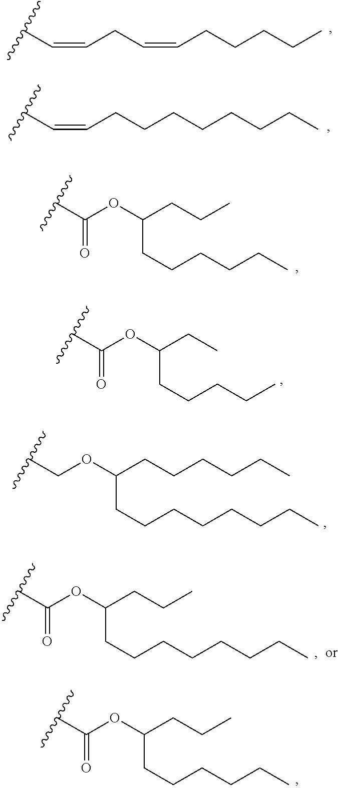

In some embodiments, R^{1A1} and R^{2A1} are each

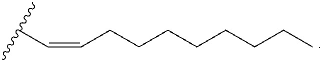 [SMILES: */C=C\CCCCCCCC]

In some embodiments, R^{1A2}, R^{1A3}, R^{2A2}, and R^{2A3} are each H.

In some embodiments, R^{1A1} and R^{2A1} are each C₁₋₁₅ alkyl; R^{1A2} and R^{2A2} are each C₁₋₁₅ alkyl; and R^{1A3} and R^{2A3} are each H. In some embodiments, R^{1A1} and R^{2A1} are each

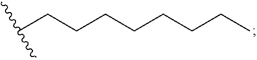 [SMILES: *CCCCCCCC]

and R^{1A2} and R^{2A2} are each

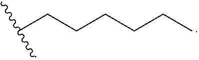 [SMILES: *CCCCCC]

In some embodiments, R^{1A3} and R^{2A3} are each H. In some embodiments, R^{1A} and R^{2A} are each a bond.

In some embodiments, R^{1A1} and R^{2A1} are each —(CH₂)₀₋₄OC(O)CH₂(C₁₋₁₅ alkyl); R^{2A1} and R^{2A2} are each —(CH₂)₀₋₄C(O)OCH₂(C₁₋₁₅ alkyl); and R^{1A3} and R^{2A3} are each H. In some embodiments, R^{1A1} and R^{2A1} are each

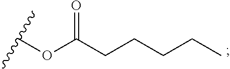 [SMILES: *OC(=O)CCCCC]

and R^{2A1} and R^{2A2} are each

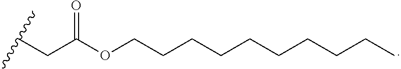 [SMILES: *CC(=O)OCCCCCCCCCC]

In some embodiments, R^{1A3} and R^{2A3} are each H. In some embodiments, R^{1A} and R^{2A} are each a bond.

In some embodiments, R^{1A1} and R^{2A1} are each —C(O)OCH₂(C₁₋₁₅ alkyl); R^{1A2} and R^{2A2} are each —(CH₂)₀₋₄C(O)OCH₂(C₁₋₁₅ alkyl); and R^{1A3} and R^{2A3} are each H. In some embodiments, R^{1A1} and R^{2A1} are each

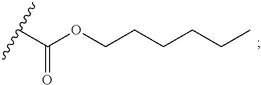 [SMILES: *C(=O)OCCCCCC]

and R^{1A2} and R^{2A2} are each

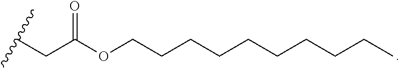 [SMILES: *CC(=O)OCCCCCCCCCC]

In some embodiments, R^{1A1} and R^{2A1} are each

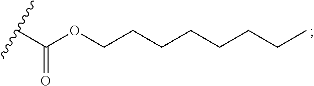 [SMILES: *C(=O)OCCCCCCCC]

and R^{2A1} and R^{2A2} are each

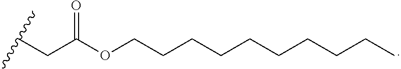 [SMILES: *CC(=O)OCCCCCCCCCC]

In some embodiments, R^{1A3} and R^{2A3} are each H. In some embodiments, R^{1A} and R^{2A} are each a bond.

In some embodiments, R^{3A1}, R^{3A2}, and R^{3A3} are each independently H, C₁₋₁₅ alkyl, —(CH₂)₀₋₄C(O)OCH(C₁₋₅ alkyl)(C₁₋₁₀ alkyl), —(CH₂)₀₋₄OC(O)CH(C₁₋₅ alkyl)(C₁₋₁₀ alkyl), —(CH₂)₀₋₄C(O)OCH₂(C₁₋₁₀ alkyl), or —(CH₂)₀₋₄OC(O)CH₂(C₁₋₁₀ alkyl).

In some embodiments, R^{3A1} and R^{3A2} are each independently C₁₋₁₅ alkyl; and R^{3A3} is H. In some embodiments, R^{3A1} and R^{3A2} are each independently ethyl, propyl, butyl, pentyl, hexyl, or heptyl. In some embodiments, R^{3A1} and R^{3A2} are each independently ethyl,

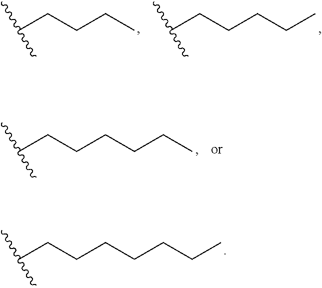

In some embodiments, R^{3A3} is H. In some embodiments, R^{3A} is a bond.

In some embodiments, R^{3A1} is C₁₋₁₅ alkyl; and R^{3A2} and R^{3A3} are each H. In some embodiments, R^{3A1} is

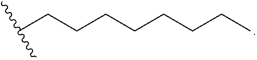 [SMILES: *CCCCCCCC]

In some embodiments, R^{3A2} and R^{3A3} are each H. In some embodiments, R^{3A} is a bond.

In some embodiments, R^{3A1} is —C(O)OCH(C₁₋₅ alkyl)(C₁₋₁₀ alkyl); and R^{3A2} and R^{3A3} are each H. In some embodiments, R^{3A1} is

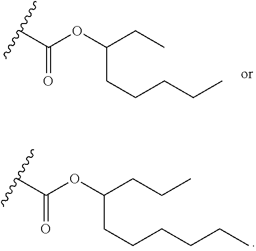 [SMILES: *C(=O)OC(CC)CCCCC ; *C(=O)OC(CCC)CCCCCC]

In some embodiments, R^{3A1} is

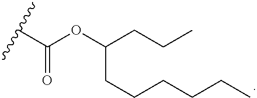 [SMILES: *C(=O)OC(CCC)CCCCCC]

In some embodiments, R^{3A} is ethylene or —(CH₂)₂—. In some embodiments, R^{3A2} and R^{3A3} are each H.

In some embodiments, R^{3A1} is —(CH₂)₀₋₄OC(O)CH₂(C₁₋₁₀ alkyl); R^{3A2} is —(CH₂)₀₋₄(O)OCH₂(C₁₋₁₀ alkyl); and R^{3A3} is H. In some embodiments, R^{3A1} is

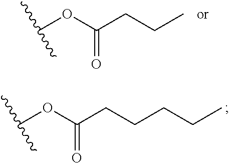

### and R^{3A2} is

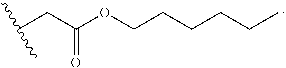 [SMILES: *CC(=O)OCCCCCC]

In some embodiments, R^{3A3} is H. In some embodiments, R^{3A} is a bond.

In some embodiments, R^{3A1} is —(CH₂)₀₋₄C(O)OCH₂(C₁₋₁₀ alkyl); R^{3A2} is —(CH₂)₀₋₄C(O)OCH₂(C₁₋₁₀ alkyl); and R^{3A3} is H. In some embodiments, R^{3A1} is

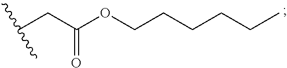 [SMILES: *CC(=O)OCCCCCC]

### and R^{3A2} is

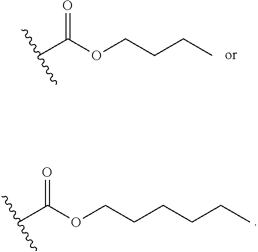

In some embodiments, R^{3A3} is H. In some embodiments, R^{3A} is a bond.

In some embodiments, R^{3A1}, R^{3A2}, and R^{3A3} are each H.
R^{a1} and R^{a2} are each independently C₁₋₂₀ alkyl or C₁₋₂₀ alkenyl. In some embodiments, R^{a1} and R^{a2} are each independently —(CH₂)₀₋₁₅CH₃ or —CH(C₁₋₁₀ alkyl)(C₁₋₁₅ alkyl). In some embodiments, R^{a1} and R^{a2} are each independently

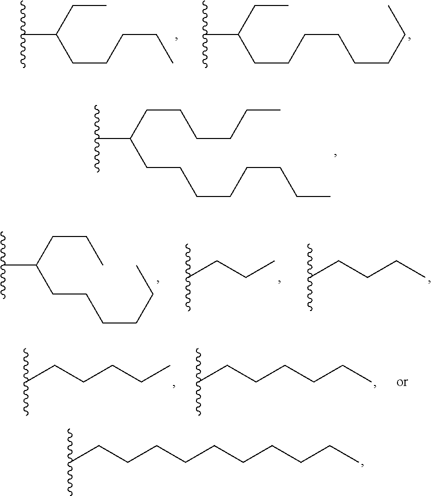

each of which is optionally substituted. In some embodiments, R^{a1} and R^{a2} are each independently unsubstituted or substituted. In some embodiments, R^{a1} and R^{a2} are unsubstituted.

In some embodiments, R^{3B} is

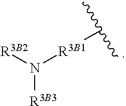

In some embodiments, R^{3B} is H. In some embodiments, R^{3B} is unsubstituted or substituted. In some embodiments, R^{3B} is unsubstituted.

In some embodiments, R^{3B1} is C₁₋₆ alkylene. In some embodiments, R^{3B1} is ethylene or propylene. In some embodiments, R^{3B1} is unsubstituted or substituted. In some embodiments, R^{3B1} is optionally substituted.

In some embodiments, R^{3B2} and R^{3B3} are each independently and optionally substituted. In some embodiments, R^{3B2} and R^{3B3} are each independently H or C₁-6 alkyl optionally substituted with one or more substituents each independently selected from the group consisting of —OH and —O—(C₁₋₆ alkyl). In some embodiments, R^{3B2} and R^{3B3} are each independently H or C₁₋₆ alkyl optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13}, and —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14} and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted. In some embodiments, R^{3B2} and R^{3B3} are each independently H, methyl, ethyl, propyl, butyl, or pentyl, each of which is optionally substituted with one or more substituents each independently selected from the group consisting of —OH and —O—(C₁₋₆ alkyl). In some embodiments, R^{3B2} and R^{3B3} are each independently methyl or ethyl, each optionally substituted with one or more —OH. In some embodiments, R^{3B2} and R^{3B3} are each methyl or each ethyl, each optionally substituted with one or more —OH. In some embodiments, R^{3B2} and R^{3B3} are each unsubstituted methyl.

In some embodiments,

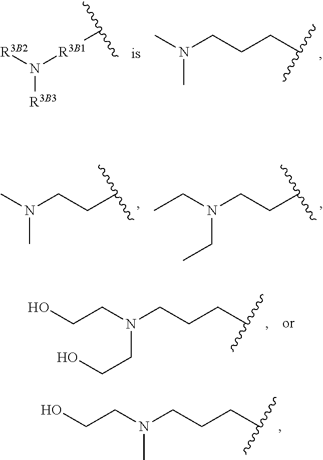

each of which is optionally substituted.

In one aspect, the present invention provides a compound represented by Formula (Ia):

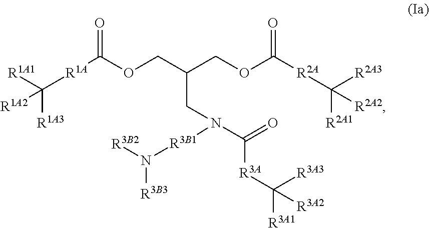

or a salt thereof, wherein R^{1A}, R^{2A}, R^{3A}, R^{1A1}, R^{1A2}, R^{1A3}, R^{2A1}, R^{2A2}, R^{2A3}, R^{3A1}, R^{3A2}, R^{3A3}, R^{3B1}, R^{3B2}, and R^{3B3} are as defined for Formula (I) or any variation or embodiment thereof.

In one aspect, the present invention provides a compound represented by Formula (Ib):

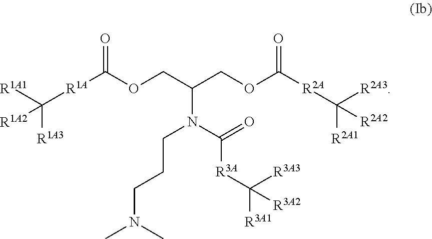

or a salt thereof, wherein R^{1A}, R^{2A}, R^{3A}, R^{1A1}, R^{1A2}, R^{1A3}, R^{2A1}, R^{2A2}, R^{2A3}, R^{3A1}, R^{3A2}, and R^{3A3} are as defined for Formula (I) or any variation or embodiment thereof.

In some embodiments, the cationic lipid in a targeted LNP of the present disclosure has a concentration between about 10 mol % to about 60 mol % of the LNP, such as about 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, or 60 mol %. In some embodiments, the cationic lipid in a targeted LNP of the present disclosure has a concentration between about 48.0 mol % to about 50.0 mol %, such as 48.0 mol %, 48.1 mol %, 48.2 mol %, 48.3 mol %, 48.4 mol %, 48.5 mol %, 48.6 mol %, 48.7 mol %, 48.8 mol %, 48.9 mol %, 49.0 mol %, 49.1 mol %, 49.2 mol %, 49.3 mol %, 49.4 mol %, 49.5 mol %, 49.6 mol %, 49.7 mol %, 49.8 mol %, 49.9 mol %, or 50.0 mol %.

In some embodiments, the cationic lipid in the targeted LNP comprises Lipid 15.

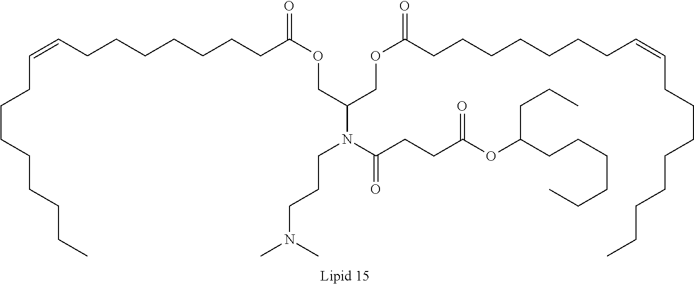 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

In some aspects, the ionizable lipid is DLin-KC2-DMA:

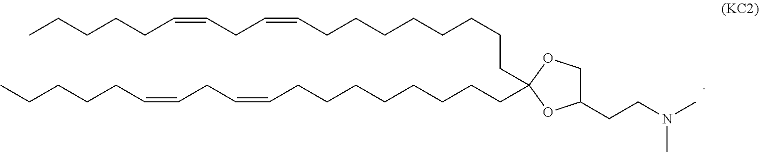 [SMILES: CCCCC/C=C\C/C=C\CCCCCCCCC1(CCCCCCCC/C=C\C/C=C\CCCCC)OCC(CCN(C)C)O1]

In some embodiments, the ionizable lipid is DLin-KC3-DMA:

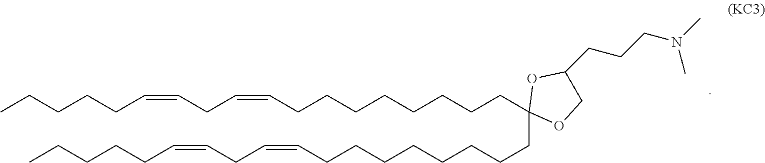 [SMILES: CCCCC/C=C\C/C=C\CCCCCCCCC1(CCCCCCCC/C=C\C/C=C\CCCCC)OCC(CCCN(C)C)O1]

In some embodiments, the ionizable lipid is DLin-MC3-DMA:

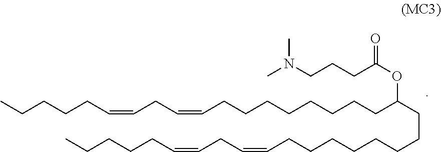 [SMILES: CCCCC/C=C\C/C=C\CCCCCCCCC(CCCCCCCC/C=C\C/C=C\CCCCC)OC(=O)CCCN(C)C]

In some embodiments, the cationic lipid, or a salt thereof, is a compound selected from the compounds listed in Table 1, or a slat thereof.

TABLE 1
(Lipid 1)

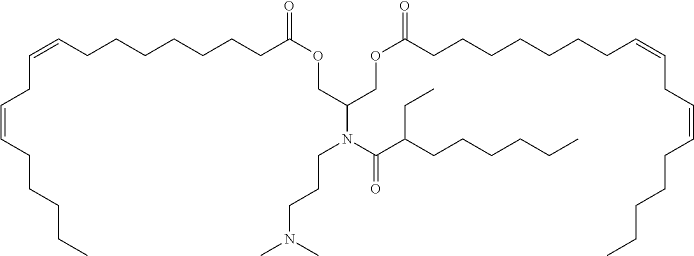 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCCC]

Lipid 1
(Lipid 2)

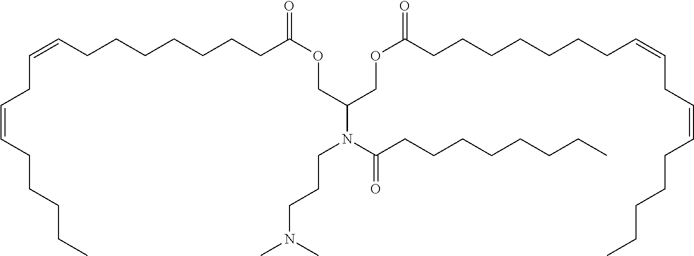 [SMILES: CCCCC/C=C\C/C=C\CCCCCCCC(=O)OCC(COC(=O)CCCCCCC/C=C\C/C=C\CCCCC)N(CCCN(C)C)C(=O)CCCCCCCC]

Lipid 2
(Lipid 3)

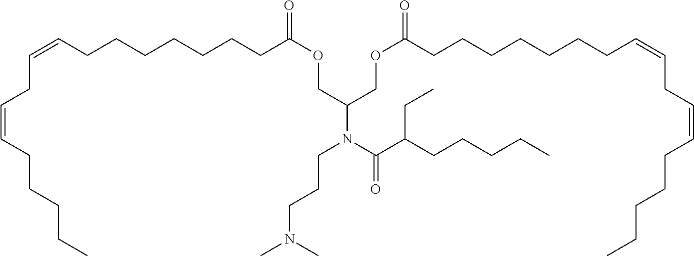 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCC]

Lipid 3
(Lipid 4)

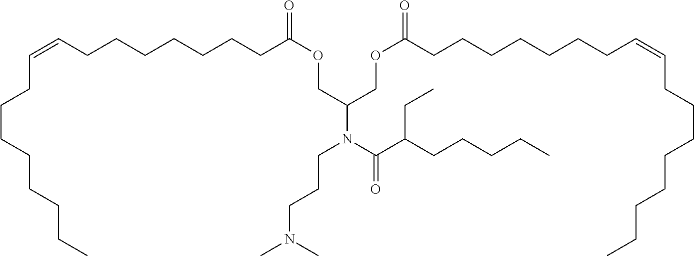 [SMILES: CCCCCCCC/C=C\CCCCCCCC(=O)OCC(COC(=O)CCCCCCC/C=C\CCCCCCCC)N(CCCN(C)C)C(=O)C(CC)CCCCC]

Lipid 4
(Lipid 5)

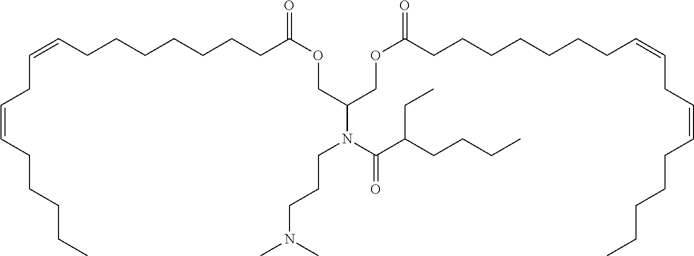 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCC]

Lipid 5
(Lipid 5A)

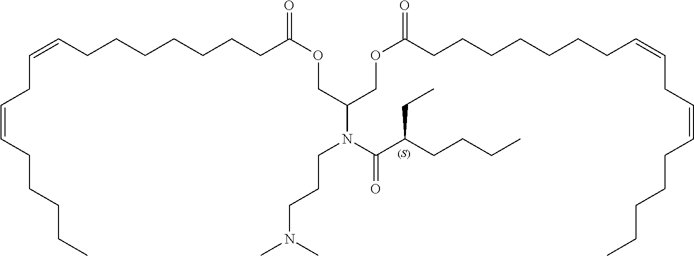

Lipid 5A
(Lipid 6)

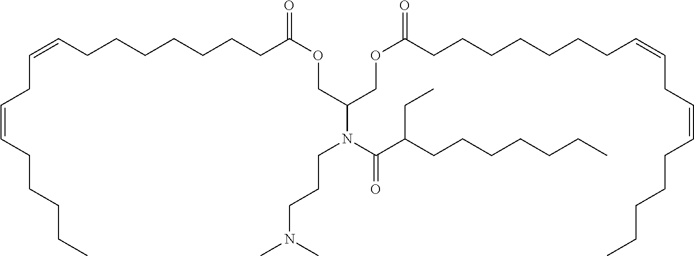 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCCCC]

Lipid 6
(Lipid 7)

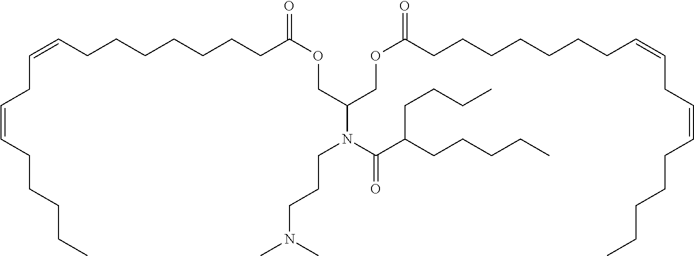 [SMILES: CCCCC/C=C\C/C=C\CCCCCCCC(=O)OCC(COC(=O)CCCCCCC/C=C\C/C=C\CCCCC)N(CCCN(C)C)C(=O)C(CCCC)CCCCC]

Lipid 7
(Lipid 8)

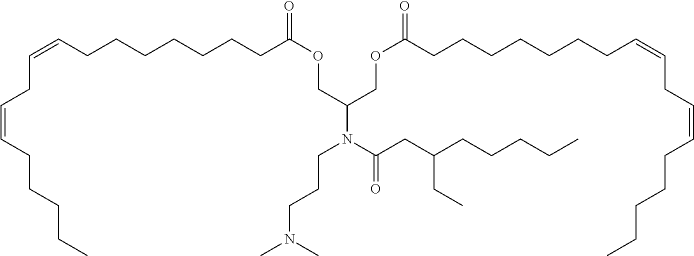 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

Lipid 8
(Lipid 9)

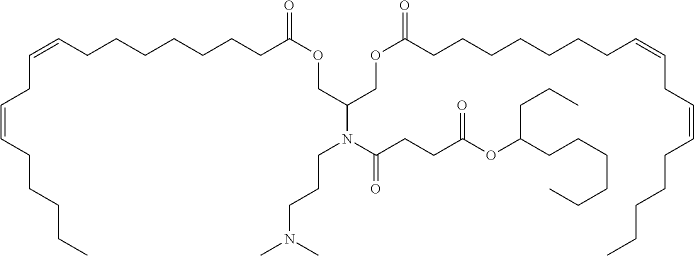 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

Lipid 9
(Lipid 10)

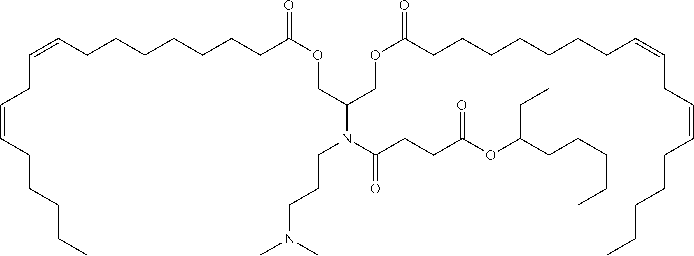 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CC)CCCCC]

Lipid 10
(Lipid 10A)

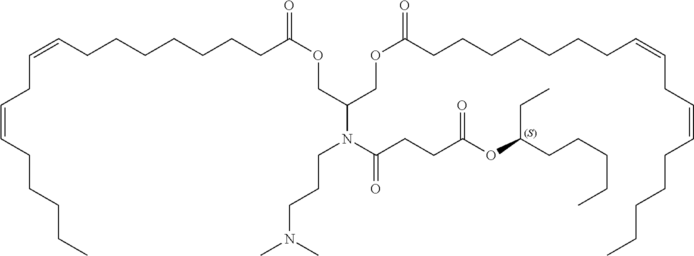

Lipid 10A
(Lipid 11)

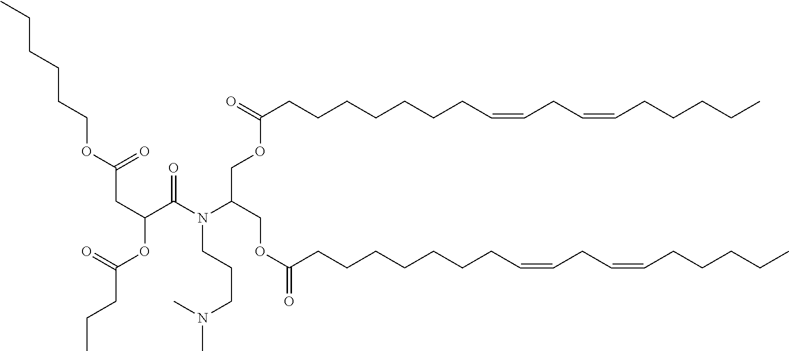 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCC]

Lipid 11
(Lipid 11A)

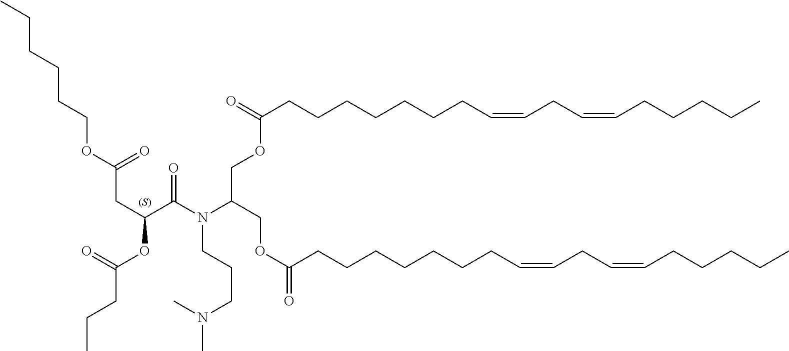

Lipid 11A
(Lipid 12)

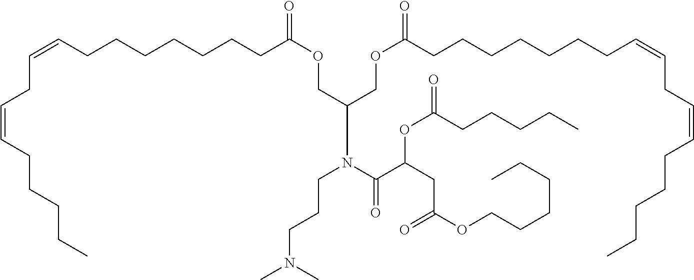 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCCCC]

Lipid 12
(Lipid 13)

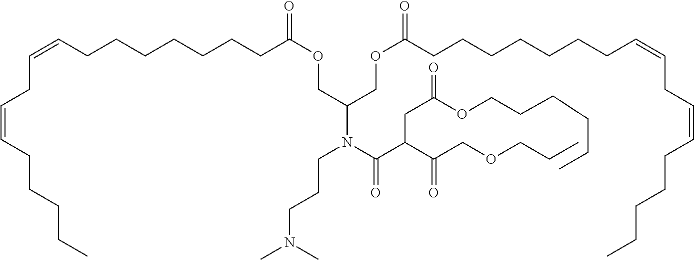 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)C(=O)COCCC]

Lipid 13
(Lipid 14)

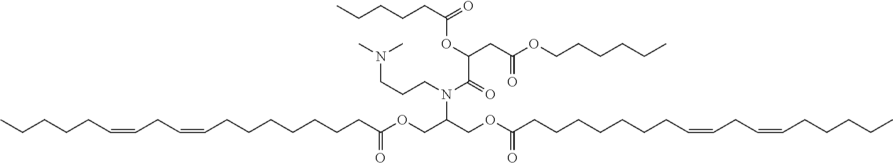 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCCCC]

Lipid 14
(Lipid 14A)

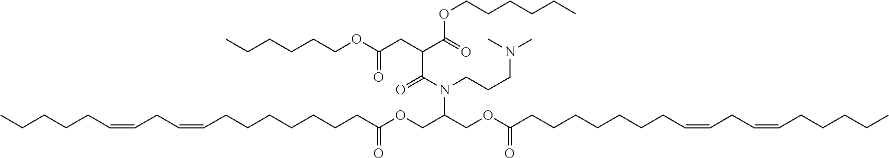 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)C(=O)OCCCCCC]

Lipid 14A
(Lipid 15)

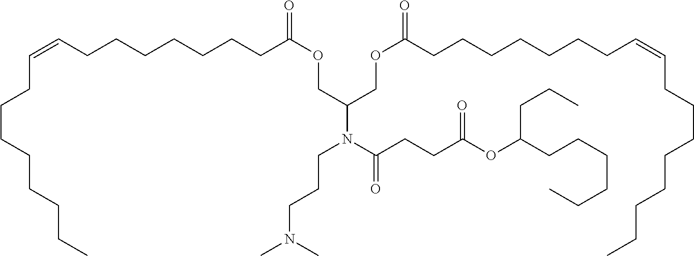 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

Lipid 15
(Lipid 16)

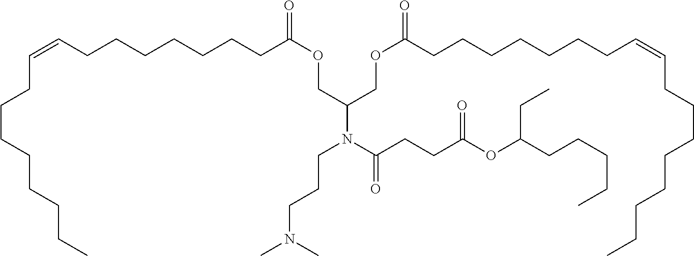 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CC)CCCCC]

Lipid 16
(Lipid 17)

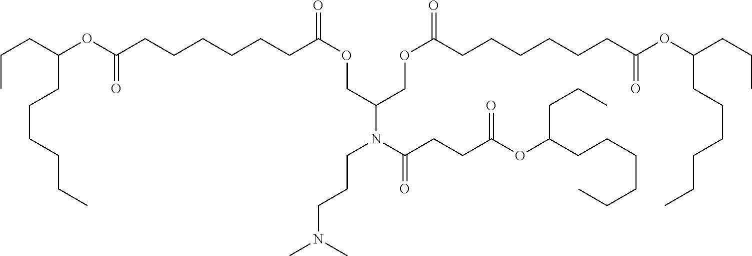 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

Lipid 17
(Lipid 17A)

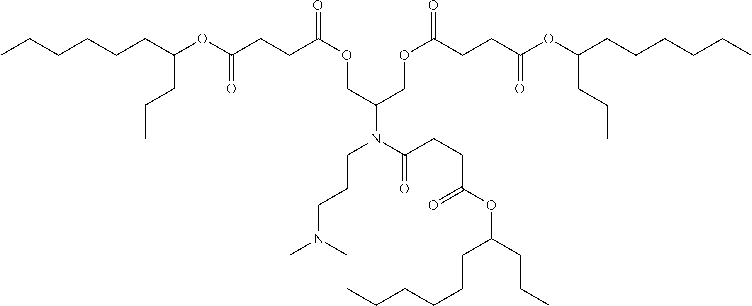 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC]

Lipid 17A
(Lipid 18)

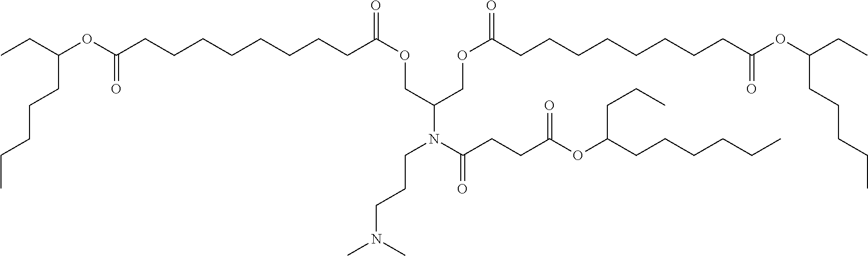 [SMILES: CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC]

Lipid 18
(Lipid 18A)

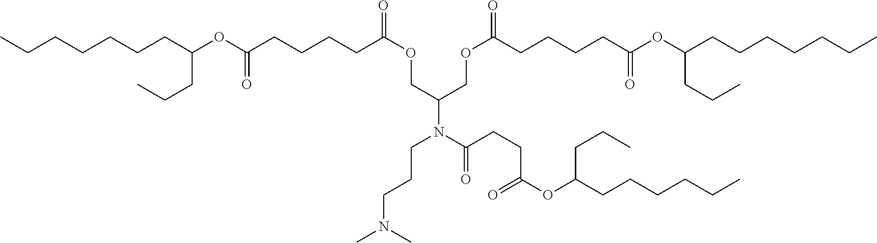 [SMILES: CCCCCCCC(CCC)OC(=O)CCCCC(=O)OCC(COC(=O)CCCCC(=O)OC(CCC)CCCCCCC)N(CCCN(C)C)C(=O)CCC(=O)OC(CCC)CCCCCC]

Lipid 18A
(Lipid 19)

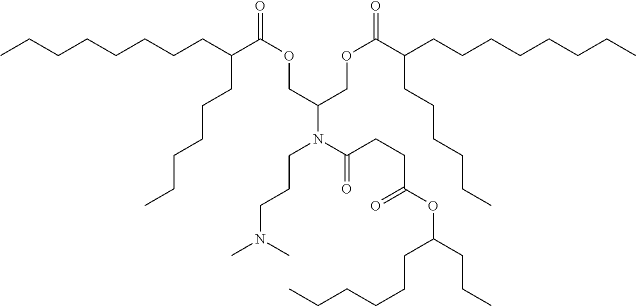 [SMILES: CCCCCCCCC(CCCCCC)C(=O)OCC(COC(=O)C(CCCCCC)CCCCCCCC)N(CCCN(C)C)C(=O)CCC(=O)OC(CCC)CCCCCC]

Lipid 19
(Lipid 19A)

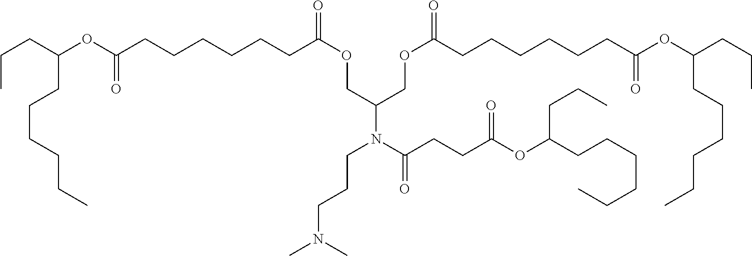 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

Lipid 19A
(Lipid 20)

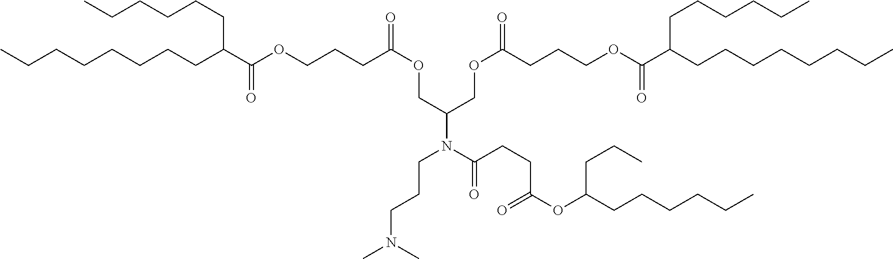 [SMILES: CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC]

Lipid 20
(Lipid 20A)

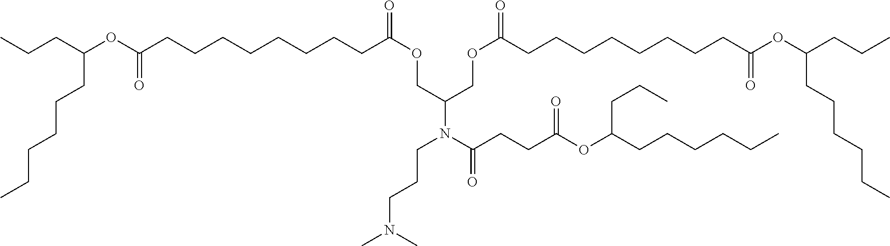 [SMILES: CCCCCCC(CCC)OC(=O)CCCCCCCCC(=O)OCC(COC(=O)CCCCCCCCC(=O)OC(CCC)CCCCCC)N(CCCN(C)C)C(=O)CCC(=O)OC(CCC)CCCCCC]

Lipid 20A
(Lipid 21)

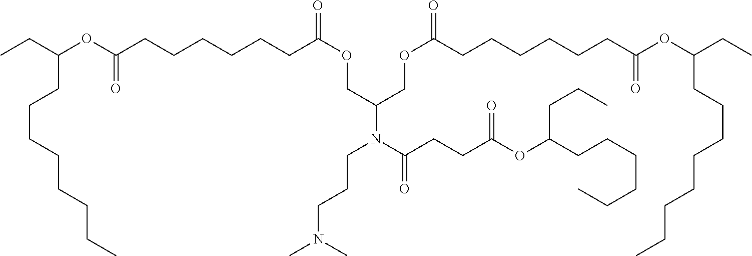 [SMILES: CCCCCCCCC(CC)OC(=O)CCCCCCC(=O)OCC(COC(=O)CCCCCCC(=O)OC(CC)CCCCCCCC)N(CCCN(C)C)C(=O)CCC(=O)OC(CCC)CCCCCC]

Lipid 21
(Lipid 21A)

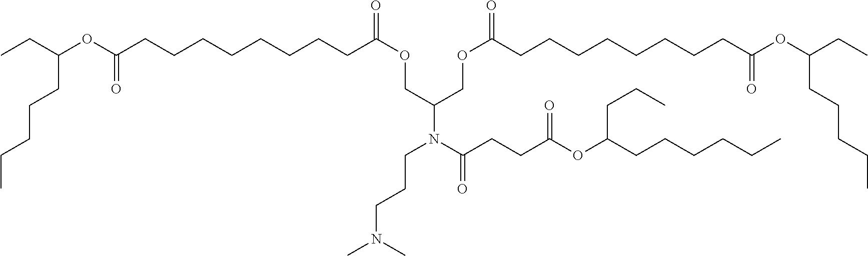 [SMILES: CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC]

Lipid 21A
(Lipid 22)

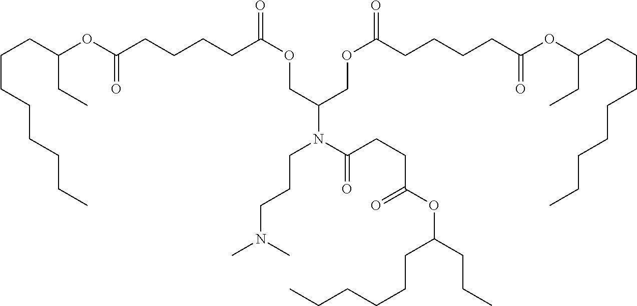 [SMILES: CCCCCCCCC(CC)OC(=O)CCCCC(=O)OCC(COC(=O)CCCCC(=O)OC(CC)CCCCCCCC)N(CCCN(C)C)C(=O)CCC(=O)OC(CCC)CCCCCC]

Lipid 22
(Lipid 23)

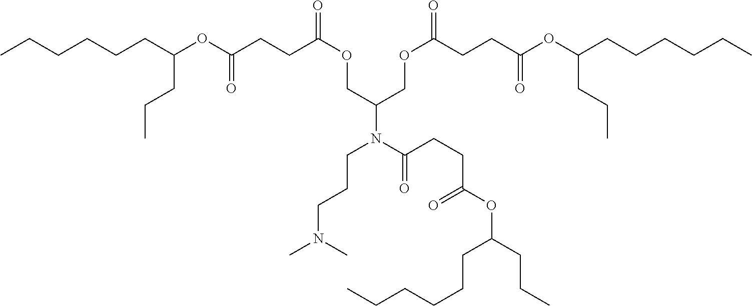 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC]

Lipid 23
(Lipid 23A)

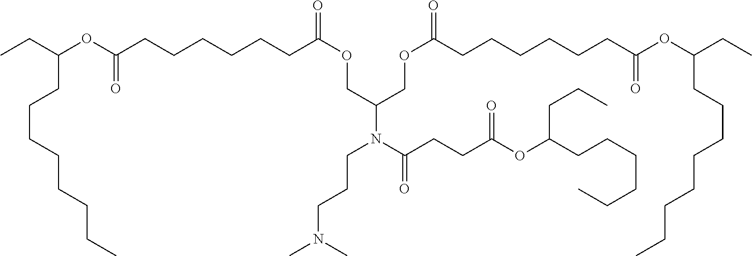 [SMILES: CCCCCCCCC(CC)OC(=O)CCCCCCC(=O)OCC(COC(=O)CCCCCCC(=O)OC(CC)CCCCCCCC)N(CCCN(C)C)C(=O)CCC(=O)OC(CCC)CCCCCC]

Lipid 23A
(Lipid 24)

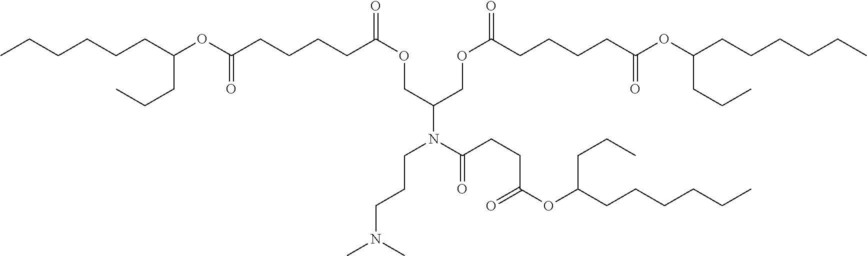 [SMILES: CCCCCCC(CCC)OC(=O)CCCCC(=O)OCC(COC(=O)CCCCC(=O)OC(CCC)CCCCCC)N(CCCN(C)C)C(=O)CCC(=O)OC(CCC)CCCCCC]

Lipid 24
(Lipid 24A)

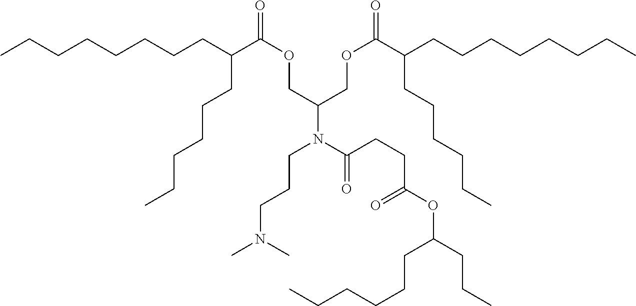 [SMILES: CCCCCCCCC(CCCCCC)C(=O)OCC(COC(=O)C(CCCCCC)CCCCCCCC)N(CCCN(C)C)C(=O)CCC(=O)OC(CCC)CCCCCC]

Lipid 24A
(Lipid 25)

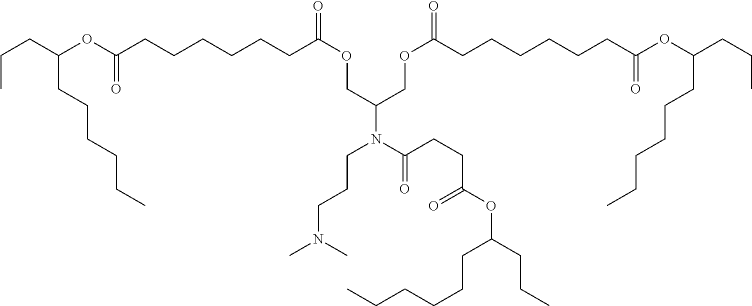 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

Lipid 25
(Lipid 25A)

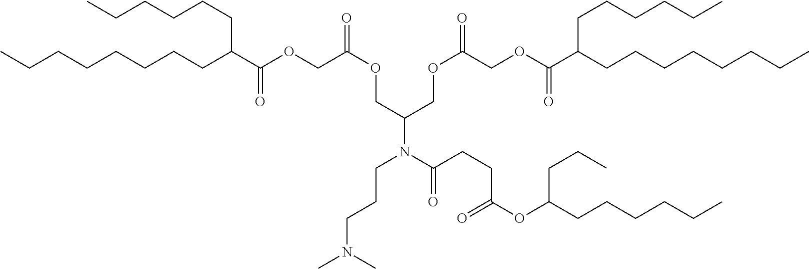 [SMILES: CCCCCCCCC(CCCCCC)C(=O)OCC(=O)OCC(COC(=O)COC(=O)C(CCCCCC)CCCCCCCC)N(CCCN(C)C)C(=O)CCC(=O)OC(CCC)CCCCCC]

Lipid 25A
(Lipid 26)

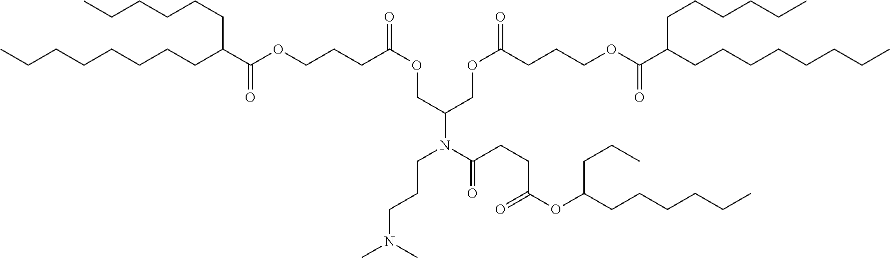 [SMILES: CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC]

Lipid 26
(Lipid 27)

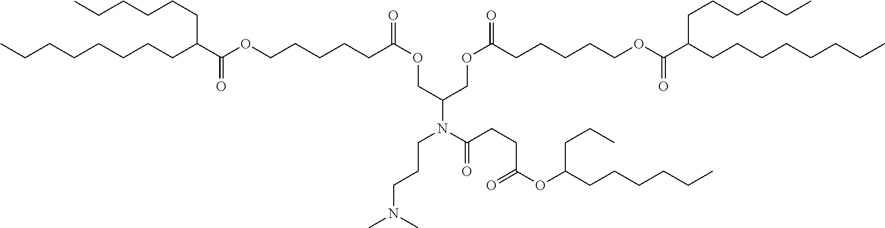 [SMILES: CCCCCCCCC(CCCCCC)C(=O)OCCCCCC(=O)OCC(COC(=O)CCCCCOC(=O)C(CCCCCC)CCCCCCCC)N(CCCN(C)C)C(=O)CCC(=O)OC(CCC)CCCCCC]

Lipid 27
(Lipid 28)

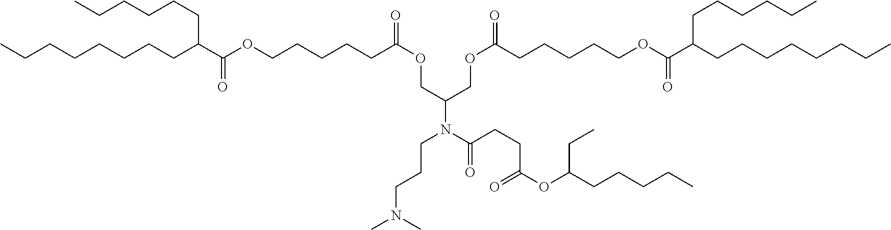 [SMILES: CCCCCCCCC(CCCCCC)C(=O)OCCCCCC(=O)OCC(COC(=O)CCCCCOC(=O)C(CCCCCC)CCCCCCCC)N(CCCN(C)C)C(=O)CCC(=O)OC(CC)CCCCC]

Lipid 28
(Lipid 29)

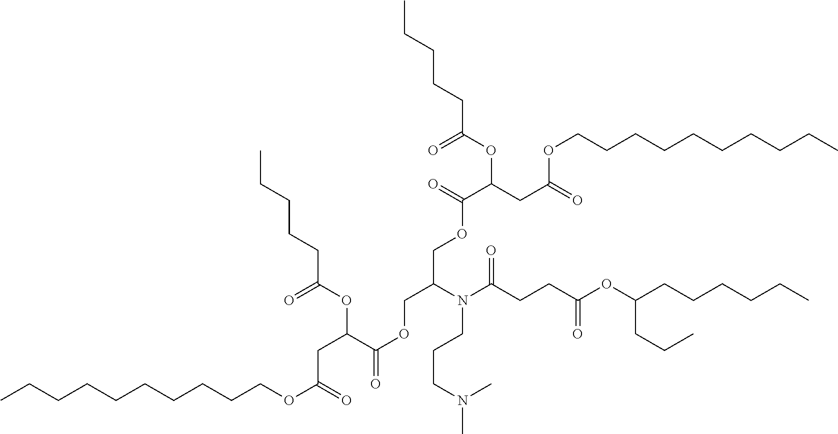 [SMILES: CCCCCCCCCCOC(=O)CC(OC(=O)CCCCC)C(=O)OCC(COC(=O)C(CC(=O)OCCCCCCCCCC)OC(=O)CCCCC)N(CCCN(C)C)C(=O)CCC(=O)OC(CCC)CCCCCC]

Lipid 29
(Lipid 30)

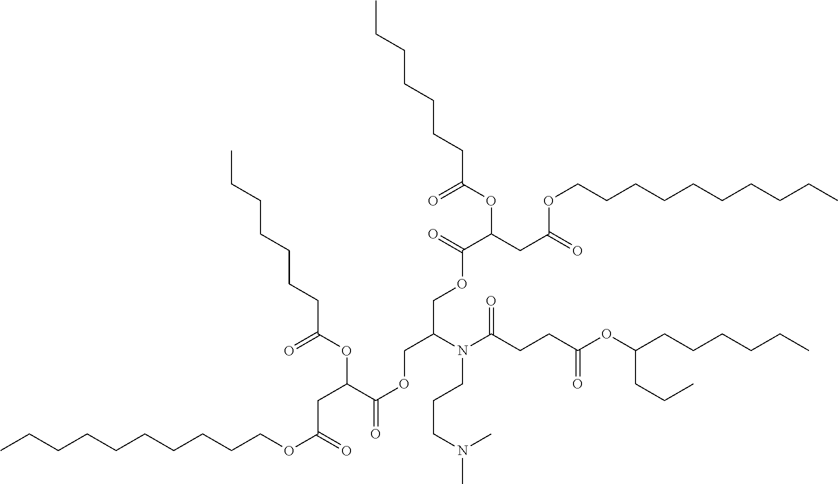 [SMILES: CCCCCCCCCCOC(=O)CC(OC(=O)CCCCCCC)C(=O)OCC(COC(=O)C(CC(=O)OCCCCCCCCCC)OC(=O)CCCCCCC)N(CCCN(C)C)C(=O)CCC(=O)OC(CCC)CCCCCC]

Lipid 30
(Lipid 31)

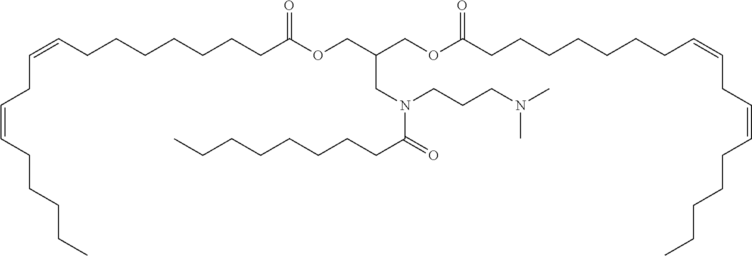 [SMILES: CCCCC/C=C\C/C=C\CCCCCCCC(=O)OCC(COC(=O)CCCCCCC/C=C\C/C=C\CCCCC)CN(CCCN(C)C)C(=O)CCCCCCCC]

Lipid 31
(Lipid 32)

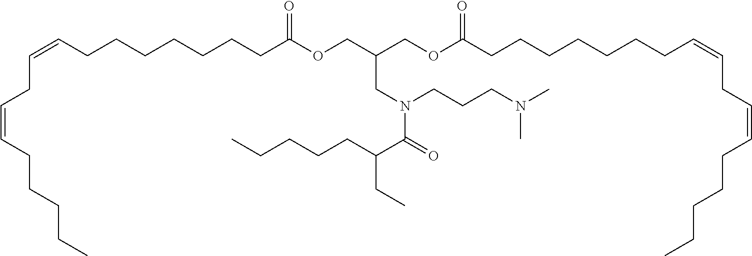 [SMILES: CCCCC/C=C\C/C=C\CCCCCCCC(=O)OCC(COC(=O)CCCCCCC/C=C\C/C=C\CCCCC)CN(CCCN(C)C)C(=O)C(CC)CCCCC]

Lipid 32
(Lipid 33)

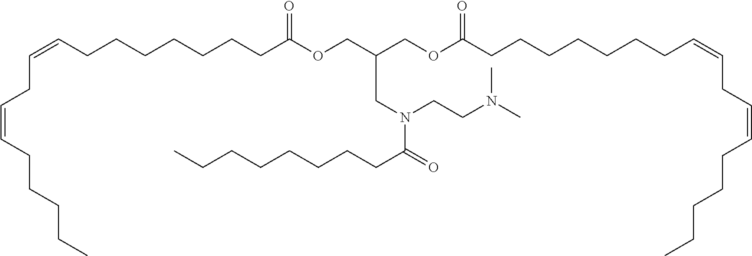 [SMILES: CCCCC/C=C\C/C=C\CCCCCCCC(=O)OCC(COC(=O)CCCCCCC/C=C\C/C=C\CCCCC)CN(CCN(C)C)C(=O)CCCCCCCC]

Lipid 33
(Lipid 34)

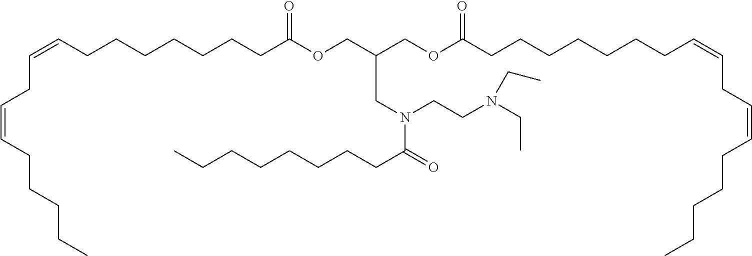 [SMILES: CCCCC/C=C\C/C=C\CCCCCCCC(=O)OCC(COC(=O)CCCCCCC/C=C\C/C=C\CCCCC)CN(CCN(CC)CC)C(=O)CCCCCCCC]

Lipid 34
(Lipid 35)

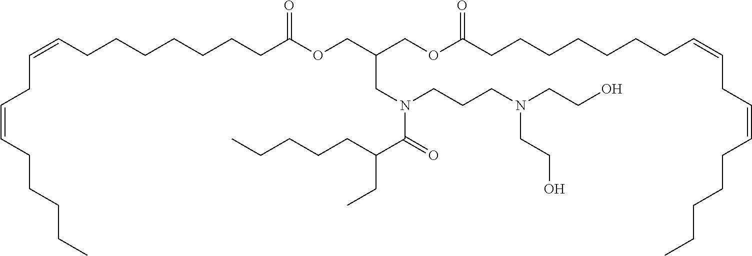 [SMILES: CCCCC/C=C\C/C=C\CCCCCCCC(=O)OCC(COC(=O)CCCCCCC/C=C\C/C=C\CCCCC)CN(CCCN(CCO)CCO)C(=O)C(CC)CCCCC]

Lipid 35
(Lipid 36)

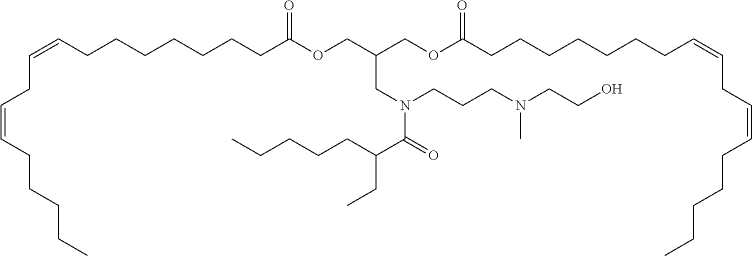 [SMILES: CCCCC/C=C\C/C=C\CCCCCCCC(=O)OCC(COC(=O)CCCCCCC/C=C\C/C=C\CCCCC)CN(CCCN(C)CCO)C(=O)C(CC)CCCCC]

Lipid 36
(Lipid 37)

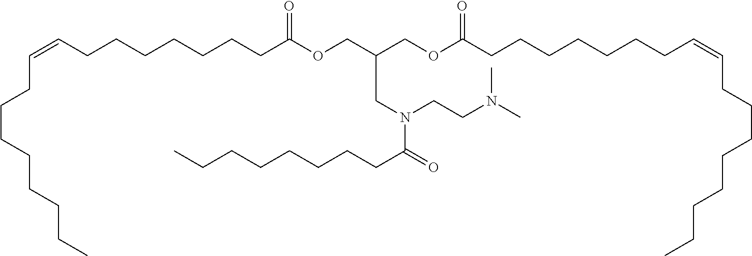 [SMILES: CCCCCCCC/C=C\CCCCCCCC(=O)OCC(COC(=O)CCCCCCC/C=C\CCCCCCCC)CN(CCN(C)C)C(=O)CCCCCCCC]

Lipid 37
(Lipid 38)

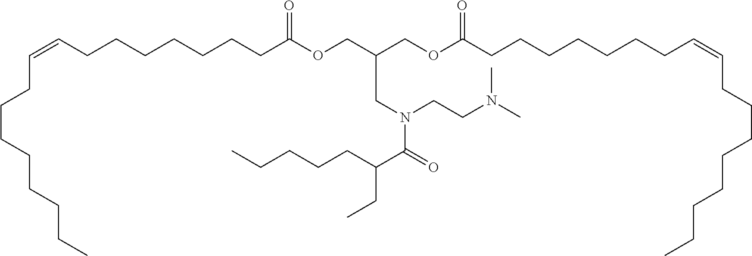 [SMILES: CCCCCCCC/C=C\CCCCCCCC(=O)OCC(COC(=O)CCCCCCC/C=C\CCCCCCCC)CN(CCN(C)C)C(=O)C(CC)CCCCC]

Lipid 38
(Lipid 37A)

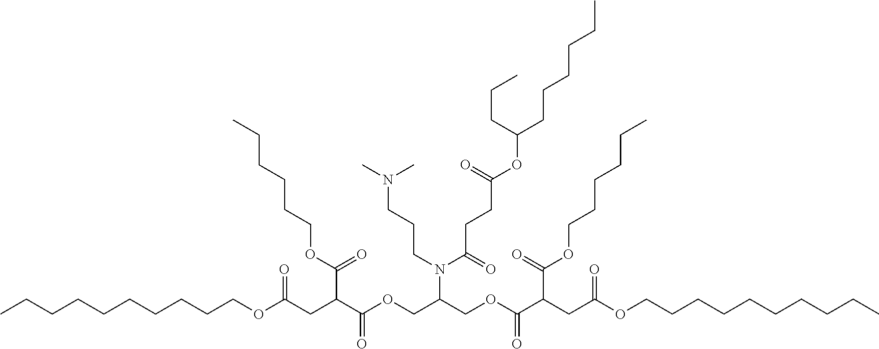 [SMILES: CCCCCCCCCCOC(=O)CC(C(=O)OCCCCCC)C(=O)OCC(COC(=O)C(CC(=O)OCCCCCCCCCC)C(=O)OCCCCCC)N(CCCN(C)C)C(=O)CCC(=O)OC(CCC)CCCCCC]

Lipid 37A
(Lipid 38A)

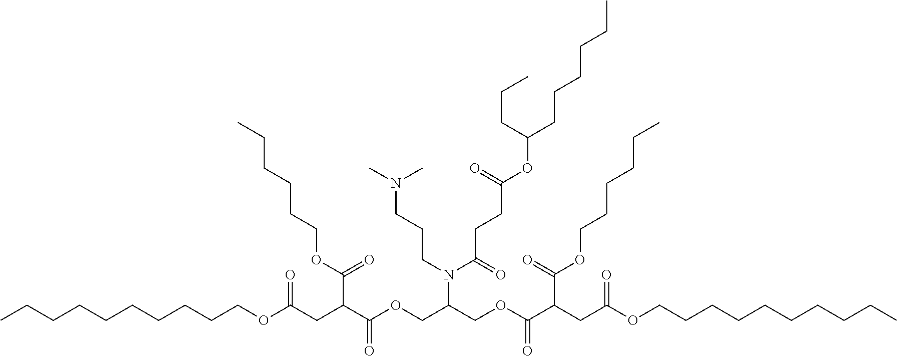 [SMILES: CCCCCCCCCCOC(=O)CC(C(=O)OCCCCCC)C(=O)OCC(COC(=O)C(CC(=O)OCCCCCCCCCC)C(=O)OCCCCCC)N(CCCN(C)C)C(=O)CCC(=O)OC(CCC)CCCCCC]

Lipid 38A

In some aspects, the ionizable lipid comprises one or more lipids as described in International PCT Publication Nos. WO2009028824A2, WO2011043913A2, WO2012091523A2, WO2015074085A1, WO2016081029A1, WO2017117530A1, WO2018118102A1, WO2018119163A1, WO2019045897A1, WO2020069445A1, WO2020106903A1, WO2020219876A1, WO2021055892A1, WO2021163339A1, WO2021188389A2, WO2021202694A1, WO2022016070A1, WO2022119883A2, WO2022120388A2, WO2022159475A1, WO2022207938A1, WO2022218957A1, WO2022246555A1, WO2023023410A2, WO2023091490A1, WO2023091787A1, WO2023114937A2, WO2023114944A1, WO2023115221A1, WO2023121970A1, WO2023121975A1, WO2023141624A1, WO2023144798A1, WO2023183616A1, WO2023196444A1, WO2023196527A2, WO2023207101A1, WO2023235589A1, WO2023240156A1, WO2024008967A1, WO2024010330A1, WO2024019770A1, and WO2024035710A2.

### C. Payload

In some embodiments, the payload is a drug substance. In some embodiments, the payload is a nucleic acid. In some embodiments, the nucleic acid is RNA. In some embodiments, the RNA is mRNA. In some embodiments, mRNA encodes a synthetic T cell receptor (synTCR) or a Chimeric Antigen Receptor (CAR). In some embodiments, mRNA comprises a 5′ Cap, a 5′ untranslated region (UTR), a payload nucleic acid sequence encoding a payload polypeptide, a 3′ UTR, and a polyA tail. In some embodiments, the payload polypeptide comprises an antibody specifically binding to a B cell, a hinge and transmembrane domain, a co-stimulatory domain, and a signaling domain.

In some embodiments, the payload is an mRNA that encodes a payload polypeptide comprising: an optional Lead peptide sequence (e.g., a signal peptide), an antibody heavy chain variable region (V_{H}), an antibody light chain variable region (V_{L}), a hinge domain, a transmembrane domain, a Co-stimulatory domain, and a Signaling domain. In some embodiments, each of the domains described herein is connected to another directly without any linker. In some embodiments, one or more linkers are used to connect any two of the domains described herein. In some embodiments, the payload is an mRNA that encodes a payload polypeptide having the following formula, arranged from N-terminus to C-terminus: [Lead peptide sequence (optional)]-[antibody heavy chain variable region (V_{HH})]-[Linker A (optional)]-[Hinge domain]-[Transmembrane domain]-[Co-stimulatory domain]-[Signaling domain]. In some embodiments, exemplary payload polypeptides are demonstrated in FIG. 41.

In some embodiments, the payload is an mRNA that comprises a 5′ Cap (optional), a 5′ UTR (optional), nucleotides encoding a Lead polypeptide sequence, nucleotides encoding a V_{HH} domain, nucleotides encoding a Linker A, nucleotides encoding a Hinge, nucleotides encoding a Transmembrane domain, nucleotides encoding Co-stimulatory domain, nucleotides encoding a Signaling domain, a 3′UTR (optional), and polyA tail (optional). In some embodiments, the payload has the following formula, arranged from 5′ to 3′: 5′ Cap (optional)-5′ UTR (optional)-nucleotides encoding Lead peptide sequence (optional)-nucleotides encoding a V_{HH}-nucleotides encoding a Linker A (optional)-nucleotides encoding a Hinge-nucleotides encoding a Transmembrane domain-nucleotides encoding Co-stimulatory domain-nucleotides encoding Signaling domain-3′UTR (optional)-poly A tail (optional).

In some embodiments, the 5′ UTR comprises SEQ ID NO: 516 or SEQ ID NO: 518. In some embodiments, the 3′ UTR comprises SEQ ID NO: 517 or SEQ ID NO: 519. In some embodiments, the 5′ UTR comprises SEQ ID NO: 516 and the 3′ UTR comprises SEQ ID NO: 517. In some embodiments, the 5′ UTR comprises SEQ ID NO: 518 and the 3′ UTR comprises SEQ ID NO: 519.

In some embodiments, an mRNA of the present disclosure comprises a 5′ cap structure. In some embodiments, the 5′ cap structure is IRES, Cap0, Cap1, ARCA, inosine, Nl-methyl-guanosine, 2′fluoro-guanosine, 7-deaza-guanosine, CleanCap™, m7 (3′OMeG) (5′) ppp (5′) (2′OMeA) pG, 8-oxo-guanosine, 2-amino-guanosine, LNA-guanosine, 2-azido-guanosine, Cap2, Cap4, CAP-003, or CAP-225. In one embodiment, the mRNA comprises a 5′-cap and wherein at least one of the uridines in the molecule is a modified uridine, preferably Nl-methyl-pseudouridine (1m Y′). In some embodiments, the mRNA comprises a 5′ cap having the sequence NpppNU, wherein the U in the 5′ cap is an unmodified uridine. In one embodiment, the 5′ cap has the sequence NpppAU with A representing a modified or unmodified adenosine nucleotide. For example, a modified nucleotide N or A 3′ to the triphosphate linkage may have a modified ribose structure such as a 2′-O-methylated ribose (Nm or Am) resulting in a so-called “Cap 1”. In contrast, a cap comprising a nucleotide N or A 3′ to the triphosphate linkage having an unmethylated ribose is usually referred to as “Cap 0”.

In some embodiments, the payload is an mRNA that encodes a chimeric antigen receptor (CAR), wherein the CAR comprises an antibody binding domain that specifically binds to B cells. In some embodiments, the antibody binding domain specifically binding to B cells is an antibody binding domain that specifically binds to human CD19 (i.e., “anti-CD19 CAR”). Exemplary anti-CD19 CAR constructs are described herein.

### (i) Anti-CD19 Antibody Binding Domain

In some embodiments, the anti-CD19 antigen binding domain specifically binds to a target epitope of a CD19 molecule. CD19 antigens may be from various animal species, including human, non-human primate, mouse, rat, rabbit, or other non-human mammals. In some embodiments, the CD19 molecule is human CD19. In some embodiments, the CD19 molecule is rhesus macaque CD19. In some embodiments, the CD19 molecule is cynomolgus monkey CD19. In some embodiments, the CD19 molecule is wild-type. In some embodiments, the CD19 molecule is a mutant (e.g., comprises one or more insertions, deletions, and/or amino acid substitutions compared to a wild-type CD19 sequence from the same species). In some embodiments, the anti-CD19 antigen binding domain cross-reacts with a human CD19 and a cynomolgus monkey CD19 and/or a rhesus macaque CD19. In some embodiments, the anti-CD19 antigen moiety binds to wild-type human CD19.

In some embodiments, the anti-CD19 antigen binding domain (e.g., full-length, sdAb, scFv, or Fab) binds to a CD19 antigen with a K_{d}≤1 μM, such as ≤100 nM, preferably ≤10 nM, more preferably ≤1 nM. For example, the K_{d} value of the anti-CD19 antigen binding domain is between about 1 nM and about 1 pM. In some embodiments, the anti-CD19 antigen binding domain binds to a human CD19 and/or a monkey (e.g., cynomolgus, rhesus macaque) CD19 with a K_{d} of from about 1×10⁻¹¹ M to about 1×10⁻⁷ M (e.g., from about 1×10⁻¹¹ M to about 1×10⁻¹⁰ M, from about 1×10⁻¹¹ M to about 1×10⁻⁹ M, from about 1×10⁻¹¹ M to about 1×10⁻⁸ M, from about 1×10⁻¹⁰ M to about 1×10⁻⁹ M, from about 1×10⁻¹⁰ M to about 1×10⁻⁸ M, from about 1×10⁻¹⁰ M to about 1×10⁻⁷ M, from about 1×10⁻⁹ M to about 1×10⁻⁷ M, from about 1×10⁻⁹ M to about 1×10⁻⁸ M, or from about 1×10⁻⁸ M to about 1×10⁻⁷ M).

The anti-CD19 antigen binding domains described herein can be of any format and derived from any suitable anti-CD19 antibodies or antigen-binding fragments thereof. For example, the anti-CD19 antigen binding domain can be selected from a full-length antibody, an scFv, a V_{H}, a V_{L}, an scFv-scFv, an Fv, a Fab, a Fab′, a (Fab′)₂, a minibody, a diabody, a domain antibody variant (dAb), a single domain antibody (sdAb or VHH), a camelid VHH antibody, a Nanobody® ISVD, a fibronectin 3 domain variant, an ankyrin repeat variant, and other antigen-specific binding domains derived from other protein scaffolds. In some embodiments, the anti-CD19 antigen binding domain is an sdAb, a VHH, or a Nanobody® ISVD. In some embodiments, the anti-CD19 antigen binding domain is a Fab. In some embodiments, the anti-CD19 antigen binding domain is an scFv. In some embodiments, the anti-CD19 antigen binding domain is humanized. In some embodiments, the anti-CD19 antigen binding domain is chimeric. In some embodiments, the anti-CD19 antigen binding domain is derived from a monoclonal antibody of mouse, rat, monkey, or rabbit. In some embodiments, the anti-CD19 antigen binding domain is derived from any anti-CD19 monoclonal antibodies known in the art. In some embodiments, the anti-CD19 antigen binding domain is derived from a fully human antibody, for example, developed using phage-display, yeast-display, or transgenic mice bearing human Ig genes. In some embodiments, the anti-CD19 antigen binding domain is a monospecific antibody, a multispecific antibody, a monovalent antibody, or a multivalent (e.g., bivalent) antibody.

In some embodiments, the anti-CD19 antigen binding domain specifically binds to both human and non-human primates (such as cynomolgus monkey and/or rhesus macaque) CD19. In some embodiments, the anti-CD19 antigen binding domain only binds to human CD19. Exemplary anti-CD19 antibodies and antigen binding fragments thereof include, but are not limited to, tafasitamab, loncastuximab tesirine, blinatumomab, MOR00208, DI-B4, FMC63, and those disclosed in: U.S. Pat. Nos. 7,968,687; 11,091,532; 11,639,386; 11,253,547; 10,533,055; 11,975,026; 11,427,633; 10,301,388; 11,364,289; WO2018161017; and WO2017055328, the content of each of which is specifically incorporated herein by reference. Chimeric antigen receptor constructs employing such anti-CD19 antigen binding domains with cross-reactivity to monkey CD19 may facilitate toxicity studies in non-human primates, which can provide more relevant safety assessments for human clinical trial candidates, without having to perform toxicity studies in chimpanzees or using surrogate molecules.

Anti-CD19 Single Domain Antibody (sdAb; VHH; Nanobody® ISVD)

In some embodiments, the antigen binding domain specifically binding to B cells comprises a single domain antibody (sdAb) comprising a VHH. In some embodiments, the anti-CD19 antigen binding domain (e.g., scFv or Fab) specifically recognizes CD19.

In some embodiments, the anti-CD19 antigen binding domain comprises a VHH comprising an amino acid sequence of SEQ ID NO: 433. In some embodiments, the anti-CD19 antigen binding domain comprises a VHH comprising an amino acid sequence of SEQ ID NO: 481. In some embodiments, the anti-CD19 antigen binding domain comprises a VHH comprising one, two, or three CDRs selected from either one of SEQ ID NO: 433 or 481.

In some embodiments, the anti-CD19 antigen binding domain comprises: (i) a VHH amino acid sequence as set forth in SEQ ID NO: 433, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 433; or (ii) a VHH sequence as set forth in SEQ ID NO: 481, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 481. In some embodiments, the anti-CD19 antigen binding domain comprises: (i) a VHH sequence as set forth in SEQ ID NO: 433; or (ii) a VHH sequence as set forth in SEQ ID NO: 481. In some embodiments, the anti-CD19 antigen binding domain comprises SEQ ID NO: 433 or SEQ ID NO: 481.

In some embodiments, the anti-CD19 antigen binding domain binds to essentially the same epitope as an antibody comprising: (i) a VHH amino acid sequence as set forth in SEQ ID NO: 433 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 433; or (ii) a VHH amino acid sequence as set forth in SEQ ID NO: 481 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 481.

In some embodiments, the anti-CD19 antigen binding domain competes for binding to a CD19 epitope with an antibody comprising: (i) a VHH amino acid sequence as set forth in SEQ ID NO: 433 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 433; or (ii) a VHH amino acid sequence as set forth in SEQ ID NO: 481 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 481.

In some embodiments, the anti-CD19 antigen binding domain comprises a VHH of any one of SEQ ID NOs: 684-739.

In some embodiments, the anti-CD19 antigen binding domain comprises a VHH comprising an amino acid sequence of SEQ ID NO: 699.

In some embodiments, the anti-CD19 antigen binding domain comprises a VHH comprising an amino acid sequence of SEQ ID NO: 739.

In some embodiments, the anti-CD19 antigen binding domain comprises a VHH comprising an amino acid sequence of SEQ ID NO: 721.

In some embodiments, the anti-CD19 antigen binding domain comprises a VHH comprising an amino acid sequence of SEQ ID NO: 687.

In some embodiments, the anti-CD19 antigen binding domain comprises a VHH comprising an amino acid sequence of SEQ ID NO: 701.

In some embodiments, the anti-CD19 antigen binding domain comprises a VHH comprising an amino acid sequence of SEQ ID NO: 706.

In some embodiments, the anti-CD19 antigen binding domain comprises a VHH comprising an amino acid sequence of SEQ ID NO: 707.

In some embodiments, the anti-CD19 antigen binding domain comprises a VHH comprising an amino acid sequence of SEQ ID NO: 714.

In some embodiments, the anti-CD19 antigen binding domain comprises a VHH comprising an amino acid sequence of SEQ ID NO: 715.

In some embodiments, the anti-CD19 antigen binding domain comprises a VHH comprising an amino acid sequence of SEQ ID NO: 718.

In some embodiments, the anti-CD19 antigen binding domain comprises a VHH comprising an amino acid sequence of SEQ ID NO: 711.
the anti-CD19 antigen binding domain comprises a VHH comprising an amino acid sequence of SEQ ID NO: 712.

In some embodiments, the anti-CD19 antigen binding domain comprises: (i) a VHH amino acid sequence as set forth in SEQ ID NO: 699, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 699; (ii) a VHH sequence as set forth in SEQ ID NO: 739, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 481; (iii) a VHH sequence as set forth in SEQ ID NO: 721, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 721; (iv) a VHH sequence as set forth in SEQ ID NO: 687, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 687; (v) a VHH sequence as set forth in SEQ ID NO: 701, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 701; (vi) a VHH sequence as set forth in SEQ ID NO: 706, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 706; (vii) a VHH sequence as set forth in SEQ ID NO: 707, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 707; (viii) a VHH sequence as set forth in SEQ ID NO: 714, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 714; (ix) a VHH sequence as set forth in SEQ ID NO: 715, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 715; or (x) a VHH sequence as set forth in SEQ ID NO: 718, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 718.

In some embodiments, the anti-CD19 antigen binding domain binds to essentially the same epitope as an antibody comprising: (i) a VHH amino acid sequence as set forth in SEQ ID NO: 699 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 699; (ii) a VHH amino acid sequence as set forth in SEQ ID NO: 739 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 739; (iii) a VHH amino acid sequence as set forth in SEQ ID NO: 721 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 721; (iv) a VHH amino acid sequence as set forth in SEQ ID NO: 687 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 687; (v) a VHH amino acid sequence as set forth in SEQ ID NO: 701 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 701; (vi) a VHH amino acid sequence as set forth in SEQ ID NO: 706 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 706; (vii) a VHH amino acid sequence as set forth in SEQ ID NO: 707 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 707; (viii) a VHH amino acid sequence as set forth in SEQ ID NO: 714 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 714; (ix) a VHH amino acid sequence as set forth in SEQ ID NO: 715 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 715; or (x) a VHH amino acid sequence as set forth in SEQ ID NO: 718 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 718.

In some embodiments, the anti-CD19 antigen binding domain competes for binding to a CD19 epitope with an antibody comprising: (i) a VHH amino acid sequence as set forth in SEQ ID NO: 699 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 699; (ii) a VHH amino acid sequence as set forth in SEQ ID NO: 739 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 739; (iii) a VHH amino acid sequence as set forth in SEQ ID NO: 721 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 721; (iv) a VHH amino acid sequence as set forth in SEQ ID NO: 687 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 687; (v) a VHH amino acid sequence as set forth in SEQ ID NO: 701 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 701; (vi) a VHH amino acid sequence as set forth in SEQ ID NO: 706 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 706; (vii) a VHH amino acid sequence as set forth in SEQ ID NO: 706 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 706; (vii) a VHH amino acid sequence as set forth in SEQ ID NO: 707 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 707; (viii) a VHH amino acid sequence as set forth in SEQ ID NO: 714 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 714; (ix) a VHH amino acid sequence as set forth in SEQ ID NO: 715 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 715; (x) a VHH amino acid sequence as set forth in SEQ ID NO: 718 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 718; (xi) a VHH amino acid sequence as set forth in SEQ ID NO: 711 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 711; or (xii) a VHH amino acid sequence as set forth in SEQ ID NO: 712 or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence set forth in SEQ ID NO: 712.

In some embodiments, the anti-CD19 antigen binding domain comprises an amino acid sequence of SEQ ID NO: 433. In some embodiments, the anti-CD19 antigen binding domain comprises an HC-CDR1, an HC-CDR2, and an HC-CDR3 of an sdAb as set forth in SEQ ID NO: 433. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 427, VHH-CDR2 is or comprises the amino acid sequence depicted in SEQ ID NO: 428, and VHH-CDR3 is or comprises the amino acid sequence depicted in SEQ ID NO: 429.

In some embodiments, the anti-CD19 antigen binding domain comprises an amino acid sequence of SEQ ID NO: 481. In some embodiments, the anti-CD19 antigen binding domain comprises an HC-CDR1, an HC-CDR2, and an HC-CDR3 of an sdAb as set forth in SEQ ID NO: 481. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 475, VHH-CDR2 is or comprises the amino acid sequence depicted in SEQ ID NO: 476, and VHH-CDR3 is or comprises the amino acid sequence depicted in SEQ ID NO: 477.

In some embodiments, the anti-CD19 antigen binding domain comprises an amino acid sequence of SEQ ID NO: 699. In some embodiments, the anti-CD19 antigen binding domain comprises an HC-CDR1, an HC-CDR2, and an HC-CDR3 of an sdAb as set forth in SEQ ID NO: 699. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 739. In some embodiments, the anti-CD19 antigen binding domain comprises an HC-CDR1, an HC-CDR2, and an HC-CDR3 of an sdAb as set forth in SEQ ID NO: 739. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the anti-CD19 antigen binding domain comprises an amino acid sequence of SEQ ID NO: 721. In some embodiments, the anti-CD19 antigen binding domain comprises an HC-CDR1, an HC-CDR2, and an HC-CDR3 of an sdAb as set forth in SEQ ID NO: 721. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the anti-CD19 antigen binding domain comprises an amino acid sequence of SEQ ID NO: 711. In some embodiments, the anti-CD19 antigen binding domain comprises an HC-CDR1, an HC-CDR2, and an HC-CDR3 as set forth in SEQ ID NO: 711. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 850, VHH-CDR2 is or comprises the amino acid sequence depicted in SEQ ID NO: 851, and VHH-CDR3 is or comprises the amino acid sequence depicted in SEQ ID NO: 852. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 857, VHH-CDR2 is or comprises the amino acid sequence depicted in SEQ ID NO: 858, and VHH-CDR3 is or comprises the amino acid sequence depicted in SEQ ID NO: 859.

In some embodiments, the anti-CD19 antigen binding domain comprises an amino acid sequence of SEQ ID NO: 723. In some embodiments, the anti-CD19 antigen binding domain comprises an HC-CDR1, an HC-CDR2, and an HC-CDR3 of an sdAb as set forth in SEQ ID NO: 723. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 724. In some embodiments, the anti-CD19 antigen binding domain comprises an HC-CDR1, an HC-CDR2, and an HC-CDR3 of an sdAb as set forth in SEQ ID NO: 724. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 725. In some embodiments, the anti-CD19 antigen binding domain comprises an HC-CDR1, an HC-CDR2, and an HC-CDR3 of an sdAb as set forth in SEQ ID NO: 725. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO:726. In some embodiments, the anti-CD19 antigen binding domain comprises an HC-CDR1, an HC-CDR2, and an HC-CDR3 of an sdAb as set forth in SEQ ID NO: 726. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 727. In some embodiments, the anti-CD19 antigen binding domain comprises an HC-CDR1, an HC-CDR2, and an HC-CDR3 of an sdAb as set forth in SEQ ID NO: 727. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 728. In some embodiments, the anti-CD19 antigen binding domain comprises an HC-CDR1, an HC-CDR2, and an HC-CDR3 of an sdAb as set forth in SEQ ID NO: 728. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 729. In some embodiments, the anti-CD19 antigen binding domain comprises an HC-CDR1, an HC-CDR2, and an HC-CDR3 of an sdAb as set forth in SEQ ID NO: 729. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 730. In some embodiments, the anti-CD19 antigen binding domain comprises an HC-CDR1, an HC-CDR2, and an HC-CDR3 of an sdAb as set forth in SEQ ID NO: 730. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 731. In some embodiments, the anti-CD19 antigen binding domain comprises an HC-CDR1, an HC-CDR2, and an HC-CDR3 of an sdAb as set forth in SEQ ID NO: 731. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 732. In some embodiments, the anti-CD19 antigen binding domain comprises an HC-CDR1, an HC-CDR2, and an HC-CDR3 of an sdAb as set forth in SEQ ID NO: 732. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the anti-CD19 antigen binding domain comprises an amino acid sequence of SEQ ID NO: 733. In some embodiments, the anti-CD19 antigen binding domain comprises an HC-CDR1, an HC-CDR2, and an HC-CDR3 of an sdAb as set forth in SEQ ID NO: 733. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 745. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 751.

In some embodiments, the anti-CD19 antigen binding domain comprises an amino acid sequence of SEQ ID NO: 734. In some embodiments, the anti-CD19 antigen binding domain comprises an HC-CDR1, an HC-CDR2, and an HC-CDR3 of an sdAb as set forth in SEQ ID NO: 734. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 746. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 752.

In some embodiments, the anti-CD19 antigen binding domain comprises an amino acid sequence of SEQ ID NO: 735. In some embodiments, the anti-CD19 antigen binding domain comprises an HC-CDR1, an HC-CDR2, and an HC-CDR3 of an sdAb as set forth in SEQ ID NO: 735. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the anti-CD19 antigen binding domain comprises an amino acid sequence of SEQ ID NO: 736. In some embodiments, the anti-CD19 antigen binding domain comprises an HC-CDR1, an HC-CDR2, and an HC-CDR3 of an sdAb as set forth in SEQ ID NO: 736. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 744. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the anti-CD19 antigen binding domain comprises an amino acid sequence of SEQ ID NO: 737. In some embodiments, the anti-CD19 antigen binding domain comprises an HC-CDR1, an HC-CDR2, and an HC-CDR3 of an sdAb as set forth in SEQ ID NO: 737. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the anti-CD19 antigen binding domain comprises an amino acid sequence of SEQ ID NO: 738. In some embodiments, the anti-CD19 antigen binding domain comprises an HC-CDR1, an HC-CDR2, and an HC-CDR3 of an sdAb as set forth in SEQ ID NO: 738. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the anti-CD19 antigen binding domain comprises an amino acid sequence of SEQ ID NO: 715. In some embodiments, the anti-CD19 antigen binding domain comprises an HC-CDR1, an HC-CDR2, and an HC-CDR3 of an sdAb as set forth in SEQ ID NO: 715. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 753, VHH-CDR2 is or comprises the amino acid sequence depicted in SEQ ID NO: 754, and VHH-CDR3 is or comprises the amino acid sequence depicted in SEQ ID NO: 755. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 756, VHH-CDR2 is or comprises the amino acid sequence depicted in SEQ ID NO: 757, and VHH-CDR3 is or comprises the amino acid sequence depicted in SEQ ID NO: 758.

In some embodiments, the anti-CD19 antigen binding domain comprises an amino acid sequence of SEQ ID NO: 714. In some embodiments, the anti-CD19 antigen binding domain comprises an HC-CDR1, an HC-CDR2, and an HC-CDR3 of an sdAb as set forth in SEQ ID NO: 714. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 759, VHH-CDR2 is or comprises the amino acid sequence depicted in SEQ ID NO: 760, and VHH-CDR3 is or comprises the amino acid sequence depicted in SEQ ID NO: 761. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 762, VHH-CDR2 is or comprises the amino acid sequence depicted in SEQ ID NO: 763, and VHH-CDR3 is or comprises the amino acid sequence depicted in SEQ ID NO: 764.

In some embodiments, the anti-CD19 antigen binding domain comprises an amino acid sequence of SEQ ID NO: 707. In some embodiments, the anti-CD19 antigen binding domain comprises an HC-CDR1, an HC-CDR2, and an HC-CDR3 of an sdAb as set forth in SEQ ID NO: 707. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 765, VHH-CDR2 is or comprises the amino acid sequence depicted in SEQ ID NO: 766, and VHH-CDR3 is or comprises the amino acid sequence depicted in SEQ ID NO: 767. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 768, VHH-CDR2 is or comprises the amino acid sequence depicted in SEQ ID NO: 769, and VHH-CDR3 is or comprises the amino acid sequence depicted in SEQ ID NO: 770.

In some embodiments, the anti-CD19 antigen binding domain comprises an amino acid sequence of SEQ ID NO: 701. In some embodiments, the anti-CD19 antigen binding domain comprises an HC-CDR1, an HC-CDR2, and an HC-CDR3 of an sdAb as set forth in SEQ ID NO: 701. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 771, VHH-CDR2 is or comprises the amino acid sequence depicted in SEQ ID NO: 772, and VHH-CDR3 is or comprises the amino acid sequence depicted in SEQ ID NO: 773. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 774, VHH-CDR2 is or comprises the amino acid sequence depicted in SEQ ID NO: 775, and VHH-CDR3 is or comprises the amino acid sequence depicted in SEQ ID NO: 776.

In some embodiments, the anti-CD19 antigen binding domain comprises an amino acid sequence of SEQ ID NO: 718. In some embodiments, the anti-CD19 antigen binding domain comprises an HC-CDR1, an HC-CDR2, and an HC-CDR3 of an sdAb as set forth in SEQ ID NO: 718. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 777, VHH-CDR2 is or comprises the amino acid sequence depicted in SEQ ID NO: 778, and VHH-CDR3 is or comprises the amino acid sequence depicted in SEQ ID NO: 779. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 780, VHH-CDR2 is or comprises the amino acid sequence depicted in SEQ ID NO: 781, and VHH-CDR3 is or comprises the amino acid sequence depicted in SEQ ID NO: 782.

In some embodiments, the anti-CD19 antigen binding domain comprises an amino acid sequence of SEQ ID NO: 706. In some embodiments, the anti-CD19 antigen binding domain comprises an HC-CDR1, an HC-CDR2, and an HC-CDR3 of an sdAb as set forth in SEQ ID NO: 706. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 783, VHH-CDR2 is or comprises the amino acid sequence depicted in SEQ ID NO: 784, and VHH-CDR3 is or comprises the amino acid sequence depicted in SEQ ID NO: 785. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 786, VHH-CDR2 is or comprises the amino acid sequence depicted in SEQ ID NO: 787, and VHH-CDR3 is or comprises the amino acid sequence depicted in SEQ ID NO: 788.

In some embodiments, the anti-CD19 antigen binding domain comprises an amino acid sequence of SEQ ID NO: 687. In some embodiments, the anti-CD19 antigen binding domain comprises an HC-CDR1, an HC-CDR2, and an HC-CDR3 of an sdAb as set forth in SEQ ID NO: 687. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 789, VHH-CDR2 is or comprises the amino acid sequence depicted in SEQ ID NO: 815, and VHH-CDR3 is or comprises the amino acid sequence depicted in SEQ ID NO: 816. In some embodiments, the anti-CD19 antigen binding domain comprises an HC-CDR1, an HC-CDR2, and an HC-CDR3 according to the Abm definition, wherein VHH-CDR1 is or comprises the amino acid sequence depicted in SEQ ID NO: 817, VHH-CDR2 is or comprises the amino acid sequence depicted in SEQ ID NO: 818, and VHH-CDR3 is or comprises the amino acid sequence depicted in SEQ ID NO: 819.

In some embodiments, the anti-CD19 antigen binding domain comprises an amino acid sequence of SEQ ID NO: 688. In some embodiments, the anti-CD19 antigen binding domain comprises an HC-CDR1, an HC-CDR2, and an HC-CDR3 as set forth in SEQ ID NO: 688. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 841, VHH-CDR2 is or comprises the amino acid sequence depicted in SEQ ID NO: 842, and VHH-CDR3 is or comprises the amino acid sequence depicted in SEQ ID NO: 843.

In some embodiments, the anti-CD19 antigen binding domain comprises an amino acid sequence of SEQ ID NO: 700. In some embodiments, the anti-CD19 antigen binding domain comprises an HC-CDR1, an HC-CDR2, and an HC-CDR3 as set forth in SEQ ID NO: 700. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 844, VHH-CDR2 is or comprises the amino acid sequence depicted in SEQ ID NO: 845, and VHH-CDR3 is or comprises the amino acid sequence depicted in SEQ ID NO: 846.

In some embodiments, the anti-CD19 antigen binding domain comprises an amino acid sequence of SEQ ID NO: 688. In some embodiments, the anti-CD19 antigen binding domain comprises an HC-CDR1, an HC-CDR2, and an HC-CDR3 as set forth in SEQ ID NO: 688. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 841, VHH-CDR2 is or comprises the amino acid sequence depicted in SEQ ID NO: 842, and VHH-CDR3 is or comprises the amino acid sequence depicted in SEQ ID NO: 843.

In some embodiments, the anti-CD19 antigen binding domain comprises an amino acid sequence of SEQ ID NO: 710. In some embodiments, the anti-CD19 antigen binding domain comprises an HC-CDR1, an HC-CDR2, and an HC-CDR3 as set forth in SEQ ID NO: 710. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 847, VHH-CDR2 is or comprises the amino acid sequence depicted in SEQ ID NO: 848, and VHH-CDR3 is or comprises the amino acid sequence depicted in SEQ ID NO: 849.

In some embodiments, the anti-CD19 antigen binding domain comprises an amino acid sequence of SEQ ID NO: 713. In some embodiments, the anti-CD19 antigen binding domain comprises an HC-CDR1, an HC-CDR2, and an HC-CDR3 as set forth in SEQ ID NO: 713. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 853, VHH-CDR2 is or comprises the amino acid sequence depicted in SEQ ID NO: 854, and VHH-CDR3 is or comprises the amino acid sequence depicted in SEQ ID NO: 429.

In some embodiments, the anti-CD19 antigen binding domain comprises an amino acid sequence of SEQ ID NO: 719. In some embodiments, the anti-CD19 antigen binding domain comprises an HC-CDR1, an HC-CDR2, and an HC-CDR3 as set forth in SEQ ID NO: 719. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 855, and VHH-CDR3 is or comprises the amino acid sequence depicted in SEQ ID NO: 856.

In some embodiments, the anti-CD19 antigen binding domain comprises an amino acid sequence of SEQ ID NO: 717. In some embodiments, the anti-CD19 antigen binding domain comprises an HC-CDR1, an HC-CDR2, and an HC-CDR3 as set forth in SEQ ID NO: 717. In some embodiments, the anti-CD19 antigen binding domain comprises an HC-CDR1, an HC-CDR2, and an HC-CDR3 according to the Kabat definition, wherein VHH-CDR1 is or comprises the amino acid sequence depicted in SEQ ID NO: 753, VHH-CDR2 is or comprises the amino acid sequence depicted in SEQ ID NO: 754, and VHH-CDR3 is or comprises the amino acid sequence depicted in SEQ ID NO: 755.

In some embodiments, the anti-CD19 antigen binding domain is selected from the antibodies below:

TABLE D
Anti-CD19 antibodies, as defined by Kabat numbering.

| | | | SEQ |
|---|---|---|---|
| | | | ID |
| | | Sequences | NO: |
| Anti- | HC- | DYTLA | 427 |
| CD19 | CDR1 | | |
| mAb-1 | HC | VSTWNRYTYYAEGVKG | 428 |
| (αCD19- | CDR2 | | |
| A clone) | HC- | KFRISYNVRDDSFYDL | 429 |
| (also | CDR3 | | |
| referred | VHH | EVQLVESGGGVVQPGGSLRLSCAASGLFLSDYTLAWFRQAPGKERE | 433 |
| to as | | FVIVSTWNRYTYYAEGVKGRFTISRDNAKKTVYLQMNSLRPEDTAL | |
| VHH-A) | | YYCAAKFRISYNVRDDSFYDLWGQGTLVTVSS | |
| Anti- | HC- | TKAMG | 475 |
| CD19 | CDR1 | | |
| mAb-2 | HC- | AITWTAGLTYYADSVKG | 476 |
| (αCD19- | CDR2 | | |
| B clone) | HC- | RRTFSYEHPNYYTRSAYHF | 477 |
| (also | CDR3 | | |
| referred | VHH | EVQLVESGGGVVQPGGSLRLSCAASGDTFGTKAMGWFRQAPGKGRE | 481 |
| to as | | FVAAITWTAGLTYYADSVKGRFTISKDRAKNTVDLQMNSLRPEDTAL | |
| VHH-B) | | YYCASRRTFSYEHPNYYTRSAYHFWGQGTLVTVSS | |
| VHH-H | HC- | SYAMA | 740 |
| | CDR1 | | |
| | HC- | VISLSGGSVDYRDSVKG | 741 |
| | CDR2 | | |
| | HC- | RPVYMKWSERDFDY | 743 |
| | CDR3 | | |
| | VHH | EVQLVESGGGLVQAGGSLRLSCAASGFTFSSYAMAWFRQAPGK | 699 |
| | | EREFVAVISLSGGSVDYRDSVKGRFTISRDNAQSTVSLQMNNL | |
| | | KPEDTAIYYCAARPVYMKWSERDFDYWGQGTLVTVSS | |
| VHH- | HC- | SYAMA | 740 |
| AV | CDR1 | | |
| | HC- | VISLSGGSVDYRDSVKG | 741 |
| | CDR2 | | |
| | HC- | RPVYIKWSERDFDY | 744 |
| | CDR3 | | |
| | VHH | EVQLVESGGGVVQPGGSLRLSCAASGFTFSSYAMAWFRQAP | 721 |
| | | GKEREFVAVISLSGGSVDYRDSVKGRFTISRDNAKNTVDLQM | |
| | | NSLRPEDTALYYCAARPVYIKWSERDFDYWGQGTLVTVSS | |
| VHH- | HC- | SYAMA | 740 |
| AD | CDR1 | | |
| | HC- | VISLSGGSVDYRDSVKG | 741 |
| | CDR2 | | |
| | HC- | RPVYIKWSERDFDY | 744 |
| | CDR3 | | |
| | VHH | EVQLVESGGGVVQPGGSLRLSCAASGFTFSSYAMAWFRQAP | 739 |
| | | GKEREFVAVISLSGGSVDYRDSVKGRFTISRDNAKNTVSLQM | |
| | | NSLRPEDTALYYCAARPVYIKWSERDFDYWGQGTLVTVSS | |

In some embodiments, one or more modifications to the anti-CD19 antigen binding domain as described herein were made while not substantially impacting the binding affinity of the anti-CD19 single domain antibodies.

### (ii) Signal Peptide

The chimeric antigen receptors of the present disclosure may comprise a signal peptide (also known as a signal sequence) at the N-terminus of the polypeptide. In general, signal peptides are peptide sequences that target a polypeptide to the desired site in a cell. In some embodiments, the signal peptide targets the CAR to the secretory pathway of the cell and will allow for integration and anchoring of the CAR into the lipid bilayer. Signal peptides including signal sequences of naturally occurring proteins or synthetic, non-naturally occurring signal sequences, which are compatible for use in the CARs described herein, will be evident to one of skill in the art. In some embodiments, the signal peptide is derived from a molecule selected from the group consisting of CD8α, GM-CSF receptor α, and IgG1 heavy chain. In some embodiments, the signal peptide is derived from a CD8α propeptide, e.g., human CD8α. In some embodiments, the signal peptide is derived from an immunoglobulin propeptide, such as an immunoglobulin light chain (e.g., Igκ). In some embodiments, the signal peptide is derived from a GM-CSF peptide, such as human GM-CSF.

In some embodiments, the anti-CD19 VHH comprises a signal peptide sequence at the N-terminus. In some embodiments, the anti-CD19 VHH comprises a signal peptide, such as a signal peptide derived from CD8α. In some embodiments, the signal peptide comprises the amino acid sequence MALPVTALLLPLALLLHAARP (SEQ ID NO: 515, CD8α signal peptide), the amino acid sequence MDMRVPAQLLGLLLLWLPGAKC (SEQ ID NO: 520, Igk signal peptide), or the amino acid sequence MWLQNLLLLGAVVCSIS (SEQ ID NO: 430, GM-CSF signal peptide). In some embodiments, the signal peptide comprises the amino acid sequence METDTLLLWVLLLWVPGSTG (SEQ ID NO: 840).

(iii) Peptide Linker

The chimeric antigen receptors of the present disclosure may comprise one or more peptide linkers, such as between different components of the CAR (e.g., between two or more intracellular co-stimulatory signaling domains, between intracellular co-stimulatory signaling domain and primary intracellular signaling domain, or between the anti-CD19 antigen binding domain and the transmembrane domain), and/or within one CAR component (e.g., antigen binding domain, such as within an scFv, or for connecting two or more antibody moieties in tandem).

Each peptide linker in a CAR may have the same or different length and/or sequence depending on the structural and/or functional features of the anti-CD19 antigen binding domain and/or the various CAR domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. For example, longer peptide linkers may be selected to ensure that two adjacent domains do not sterically interfere with one another. A short peptide linker may be disposed between the transmembrane domain and the primary intracellular signaling domain of a CAR, or between the transmembrane domain and the intracellular co-stimulatory signaling domain of a CAR. In some embodiment, the peptide linker comprises flexible residues (such as glycine and serine) so that the adjacent domains are free to move relative to each other. For example, a glycine-serine doublet can be a suitable peptide linker.

The peptide linker can be of any suitable length. The peptide linker may be at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids (aa) long. The peptide linker may be no more than about any of 100, 75, 50, 40, 35, 30, 25, 20, 19, 18, 17, 16, 15, 14, 13, 12, 11, 10, 9, 8, 7, 6, 5 or fewer aa long. In some embodiments, the length of the peptide linker is any of about 1 aa to about 10 aa, about 1 aa to about 20 aa, about 1 aa to about 30 aa, about 5 aa to about 15 aa, about 10 aa to about 25 aa, about 5 aa to about 30 aa, about 10 aa to about 30 aa, about 30 aa to about 50 aa, about 50 aa to about 100 aa, or about 1 aa to about 100 aa. In some embodiments, the peptide linker is about 10 aa to about 20 aa, such as about 15 aa.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G) n, glycine-serine polymers, glycine-alanine polymers, alanine-serine polymers, threonine-serine, and other flexible linkers known in the art. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the chimeric receptors provided herein, the disclosure of each of which is incorporated herein by reference in their entirety.

In some embodiments, the anti-CD19 antigen binding domain and the Hinge domain are connected through a linker sequence (Linker A). In some embodiments, Linker A comprises (AAA) n. In some embodiments, Linker A is AAA. In some embodiments, Linker A comprises AS. In some embodiments, Linker A is AS.

### (iv) Transmembrane Domain

The chimeric antigen receptors of the present disclosure comprise a transmembrane domain that can be directly or indirectly fused to the extracellular anti-CD19 antigen binding domain. Any protein structure that is thermodynamically stable in a cell membrane, such as a eukaryotic cell membrane, can be used as the transmembrane domain herein. The transmembrane domain may be derived either from a natural or from a synthetic source. For example, it can be a synthetic, non-naturally occurring protein segment, e.g., a hydrophobic protein segment that is thermodynamically stable in a cell membrane. The transmembrane domain can be derived from a wild-type protein, or contains one or more mutations (e.g., insertion, deletion, and/or substitution, such as conservative substitution). A transmembrane domain containing one or more mutations can be employed to avoid mispairing with endogenous protein, e.g., endogenous CD28 complex component(s), in order to enhance correct CAR assembly and/or expression level.

In some embodiments, the transmembrane domain of the anti-CD19 CAR is derived from a Type I single-pass membrane protein. In some embodiments, transmembrane domains from multi-pass membrane proteins may also be compatible for use in the anti-CD19 CARs described herein. Multi-pass membrane proteins may comprise a complex of (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the anti-CD19 CAR is derived from a molecule selected from the group consisting of: an α, β or ζ chain of a TCR, CD28, CD38, CD45, CD4, CD5, CD8, CD9, CD16, CD22, CD33, CD37, CD64, CD80, CD86, CD134, 4-1BB (CD137), CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, Claudin-6, IL-2Rβ, IL-2Rγ, IL-7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C. In some embodiments, the transmembrane domain is derived from a molecule selected from the group consisting of CD8α and CD28. In some embodiments, the transmembrane domain is derived from CD28.

### (v) Hinge Domain

The chimeric antigen receptors of the present disclosure may comprise a hinge domain that is located between the anti-CD19 antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used. A peptide linker (e.g., any of those described in the “Peptide Linker” subsection below) can be used as a hinge domain. The hinge domain may confer stability to a multi-chain chimeric receptor, such as by forming disulfide-bond between two polypeptide chains of a chimeric receptor (e.g., a CH1-CL pair hinge domain, or an antibody hinge region pair).

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

The hinge domain can be a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α.

Non-naturally occurring peptides may also be used as hinge domains for the chimeric receptors described herein. In some embodiments, the hinge domain between the C-terminus of the extracellular antigen binding domain and the N-terminus of the transmembrane domain is a peptide linker, such as a (AAA), linker, wherein n can be an integer of at least 1, e.g., 1, 2, 3, 4, or more; or AAA or AS.

In some embodiments, the hinge and transmembrane domains are derived from the same protein (e.g., CD8α or CD28). In some embodiments, the Hinge-transmembrane region is about 5 to 10 aa long. In some embodiments, the Hinge-transmembrane region is about 10 to 20 aa long. In some embodiments, the Hinge-transmembrane region is about 20 to 30 aa long. In some embodiments, the Hinge-transmembrane region is about 30 to 40 aa long. In some embodiments, the Hinge-transmembrane region is about 40 to 50 aa long. In some embodiments, the Hinge-transmembrane region is about 50 to 60 aa long. In some embodiments, the Hinge-transmembrane region is about 60 to 70 aa long. In some embodiments, the Hinge-transmembrane region is about 70 to 80 aa long. In some embodiments, the Hinge-transmembrane region is about 80 to 90 aa long. In some embodiments, the Hinge-transmembrane region is about 90 to 100 aa long. In some embodiments, the Hinge-transmembrane region is about 100 to 120 aa long.

In some embodiments, the Hinge-transmembrane region is derived from human CD8alpha or human CD28 (see Cells 2020, 9, 1182; doi: 10.3390/cells9051182).

In some embodiments, the Hinge-transmembrane region is derived from human CD8alpha, comprising the full sequence or a functional fragment of LSNSIMYFSHFVPVFLPAKPTTTPAPRPPTPAPTIASQPLSLRPEACRPAAGGAVHTRG LDFACDIYIWAPLAGTCGVLLLSLVITLYCNHRN (SEQ ID NO: 521), such as about 10-20 amino acids, about 20-30 amino acids, about 30-40 amino acids, about 40-50 amino acids, about 50-60 amino acids, about 60-70 amino acids, about 70-80 amino acids, or about 80-90 amino acids of SEQ ID NO: 521. In some embodiments, the Hinge-transmembrane region comprises or is SEQ ID NO: 521, or share at least 90%, 91%, 92%, 93%, 94%, 95%, 96%, 97%, 98%, 99%, 99.5% with SEQ ID NO: 521. In some embodiments, the Hinge-transmembrane region comprises or is SEQ ID NO: 538, or share at least 90%, 91%, 92%, 93%, 94%, 95%, 96%, 97%, 98%, 99%, 99.5% with SEQ ID NO: 538. In some embodiments, the Hinge-transmembrane region comprises or is SEQ ID NO: 539, or share at least 90%, 91%, 92%, 93%, 94%, 95%, 96%, 97%, 98%, 99%, 99.5% with SEQ ID NO: 539. In some embodiments, the Hinge-transmembrane region comprises or is SEQ ID NO: 540, or share at least 90%, 91%, 92%, 93%, 94%, 95%, 96%, 97%, 98%, 99%, 99.5% with SEQ ID NO: 540.

In some embodiments, the Hinge-transmembrane region is derived from human CD28, comprising the full sequence or a functional fragment of IEVMYPPPYLDNEKSNGTIIHVKGKHLCPSPLFPGPSKPFWVLVVVGGVLACYSLLVT VAFIIFWV̅ (SEQ ID NO: 431) or IEVMYPPPYLDNEKSNGTIIHVKGKHLCPSPLFPGPSKPFWVLVVVGGVLACYSLLVT VAFIIFWK (SEQ ID NO: 434) or IEVMYPPPYLDNERSNGTIIHVKGKHLCPSPLFPGPSKPFWVLVVVGGVLACYSLLVT VAFIIFWV (SEQ ID NO: 522), such as about 10-20 amino acids, about 20-30 amino acids, about 30-40 amino acids, about 40-50 amino acids, about 50-60 amino acids, or about 60-70 amino acids of any one of SEQ ID NOs: 431, 434, or 522. In some embodiments, the Hinge-transmembrane region comprises or is SEQ ID NO: 431, 434, or 522. In some embodiments, the Hinge region comprises or is SEQ ID NO: 438, 439, or 541. In some embodiments, the transmembrane region comprises or is SEQ ID NO: 440 or 542, or share at least 90%, 91%, 92%, 93%, 94%, 95%, 96%, 97%, 98%, 99%, 99.5% with SEQ ID NO: 440 or 542.

### (vi) Co-Stimulatory Domain

In some embodiments, the chimeric antigen receptor of the present disclosure comprises at least one co-stimulatory domain. The term “co-stimulatory signaling domain,” as used herein, refers to at least a portion of a protein that mediates a secondary or co-stimulatory signal transduction within a cell to induce an immune response such as an effector function. The co-stimulatory signaling domain of the CAR described herein can be an intracellular signaling domain from a co-stimulatory protein, which transduces a secondary or co-stimulatory signal and modulates responses mediated by immune cells, such as T cells. In some embodiments, the co-stimulatory domain is derived from a protein selected from the group consisting of: CD27, CD28, 4-1BB (also known as CD137), OX40, CD40, PD-1, LFA-1, ICOS, CD2, CD7, LIGHT, B7-H3, TNFRSF9, TNFRSF4, TNFRSF8, CD40LG, ITGB2, KLRC2, TNFRSF18, and TNFRSF14.

In some embodiments, the co-stimulatory (co-stim) domain is derived from CD28. In some embodiments, the co-stim domain comprises the full sequence or a functional fragment of RSKRSRLLHSDYMNMTPRRPGPTRKHYQPYAPPRDFAAYRS (SEQ ID NO: 543). In some embodiments, the transmembrane region comprises or is SEQ ID NO: 543, or share at least 90%, 91%, 92%, 93%, 94%, 95%, 96%, 97%, 98%, 99%, 99.5% with SEQ ID NO: 543.

In some embodiments, the co-stimulatory (co-stim) domain is derived from 4-1BB. In some embodiments, the co-stim domain comprises the full sequence or a functional fragment of RGRKKLLYIFKQPFMRPVQTTQEEDGCSCRFPEEEEGGCEL (SEQ ID NO: 432). In some embodiments, the transmembrane region comprises or is SEQ ID NO: 432, or share at least 90%, 91%, 92%, 93%, 94%, 95%, 96%, 97%, 98%, 99%, 99.5% with SEQ ID NO: 432.

In some embodiments, the anti-CD19 CAR comprises (or consists essentially of, or consists of) a single co-stimulatory domain. In some embodiments, the anti-CD19 CAR comprises (or consists essentially of, or consists of) two or more (such as about any of 2, 3, 4, or more) co-stimulatory domains. In some embodiments, the anti-CD19 CAR comprises two or more of the same co-stimulatory domains. In some embodiments, the anti-CD19 CAR comprises two or more co-stimulatory domains from different co-stimulatory proteins, such as any two or more co-stimulatory proteins described herein.

In some embodiments, the one or more co-stimulatory domains and the intracellular signaling domain (such as intracellular signaling domain of CD32) are fused to each other via optional peptide linkers. The intracellular signaling domain and the one or more co-stimulatory domains may be arranged in any suitable order. In some embodiments, the one or more co-stimulatory domains are located between the transmembrane domain and the intracellular signaling domain (such as intracellular signaling domain of CD32). In some embodiments, the one or more co-stimulatory domains are located at the C-terminus of the intracellular signaling domain (e.g., derived from CD32). In some embodiments, the intracellular signaling domain (e.g., derived from CD32) is located in between two co-stimulatory domains. Multiple co-stimulatory domains may provide additive or synergistic stimulatory effects.

(vii) Intracellular Signaling Domain

The intracellular signaling domain is responsible for activation of at least one of the normal effector functions of the immune effector cell expressing the chimeric antigen receptors. The term “effector function” refers to a specialized function of a cell, such as in the killing of diseased cells such as tumor cells, or in the inhibition of tumor growth and/or inhibition of tumor development, including inhibition of tumor dissemination and metastasis. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular signaling domain comprises (or consists essentially of, or consists of) a primary intracellular signaling domain of an immune effector cell. In some embodiments, the chimeric receptor comprises an intracellular signaling domain consisting essentially of a primary intracellular signaling domain of an immune effector cell. “Primary intracellular signaling domain” refers to intracellular signaling sequence that acts in a stimulatory manner to induce immune effector functions. In some embodiments, the anti-CD19 CAR intracellular signaling domain is derived from the intracellular signaling domain of a protein selected from the group consisting of: CD3ζ, FcεRIβ, FcεRIγ, FcRβ, CD3γ, CD3δ, CD3ε, CD5, CD22, CD79a, CD79b, and CD66d (CEACAM3).

In some embodiments, the intracellular signaling domain is derived from CD3 zeta. In some embodiments, the intracellular signaling domain comprises the full sequence or a functional fragment of RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO: 544), or shares at least 90%, 91%, 92%, 93%, 94%, 95%, 96%, 97%, 98%, 99%, 99.5% with SEQ ID NO: 544.

(viii) Anti-CD19 CARs

In some embodiments, the payload encodes an anti-CD19 CAR. In some embodiments, the anti-CD19 CAR comprises an amino acid sequence selected from the group consisting of SEQ ID NOs:49-51 and 435-437, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence selected from the group consisting of SEQ ID NOs:49-51 and 435-437. In some embodiments, the payload comprises an mRNA that encodes an anti-CD19 CAR as described herein, wherein the mRNA can be selected from any one of the tables below, or an mRNA encoding an amino acid sequence selected from the group consisting of SEQ ID NOs:49-51 and 435-437 and from any one of the tables below.

In some embodiments according to any of the polypeptides described above, the anti-CD19 CAR comprises the amino acid sequence of SEQ ID NO:49, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 49.

In some embodiments according to any of the polypeptides described above, the anti-CD19 CAR comprises the amino acid sequence of SEQ ID NO: 50, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 50.

In some embodiments according to any of the polypeptides described above, the anti-CD19 CAR comprises the amino acid sequence of SEQ ID NO: 51, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 51.

In some embodiments according to any of the polypeptides described above, the anti-CD19 CAR comprises the amino acid sequence of SEQ ID NO: 435, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 435.

In some embodiments according to any of the polypeptides described above, the anti-CD19 CAR comprises the amino acid sequence of SEQ ID NO: 436, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 436.

In some embodiments according to any of the polypeptides described above, the anti-CD19 CAR comprises the amino acid sequence of SEQ ID NO: 437, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 437.

In some embodiments according to any of the polypeptides described above, the anti-CD19 CAR comprises the amino acid sequence selected from the group consisting of any one of SEQ ID NOs: 576-623, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence selected from the group consisting of any one of SEQ ID Nos: 576-623.

In some embodiments according to any of the polypeptides described above, the anti-CD19 CAR comprises the amino acid sequence selected from the group consisting of any one of 624-681, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence selected from the group consisting of any one of SEQ ID Nos: 624-681.

In some embodiments according to any of the polypeptides described above, the anti-CD19 CAR comprises the amino acid sequence of SEQ ID NO: 621, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 621.

In some embodiments according to any of the polypeptides described above, the anti-CD19 CAR comprises the amino acid sequence of SEQ ID NO: 675, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 675.

In some embodiments according to any of the polypeptides described above, the anti-CD19 CAR comprises the amino acid sequence of SEQ ID NO: 584, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 584.

In some embodiments according to any of the polypeptides described above, the anti-CD19 CAR comprises the amino acid sequence of SEQ ID NO: 632, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 632.

In some embodiments according to any of the polypeptides described above, the anti-CD19 CAR comprises the amino acid sequence of SEQ ID NO: 585, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 585.

In some embodiments according to any of the polypeptides described above, the anti-CD19 CAR comprises the amino acid sequence of SEQ ID NO: 633, or an amino acid sequence comprising at least about 80% (such as at least about any of 85%, 86%, 87%, 88%, 89%, 90%, 91%, 92%, 93%, 94%, 95%, 96%, 97%, 98%, 99%, or more) sequence identity to an amino acid sequence of SEQ ID NO: 633. In some embodiments, the payload nucleic acid (e.g., payload mRNA) encoding an anti-CD19 CAR comprises a nucleic acid selected from the group consisting of SEQ ID NOs: 46-48, 441-458, 482-511, and 570-575. In some embodiments, the payload nucleic acid is a payload mRNA encoding an anti-CD19 CAR, wherein the payload mRNA comprises the RNA sequence of any one of SEQ ID NOs:570-575. In some embodiments, a nucleotide sequence encoding an anti-CD19 CAR further comprises a 5′ UTR comprising SEQ ID NO: 516 or SEQ ID NO: 518. In some embodiments, the nucleotide sequence further comprises a 3′ UTR comprising SEQ ID NO: 517 or SEQ ID NO: 519. In some embodiments, the 5′ UTR comprises SEQ ID NO: 516 and the 3′ UTR comprises SEQ ID NO: 517. In some embodiments, the 5′ UTR comprises SEQ ID NO: 518 and the 3′ UTR comprises SEQ ID NO: 519. In some embodiments, the payload mRNA comprises the RNA sequence of any one of SEQ ID NOs:573-575.

In some embodiments, the payload nucleic acid (e.g., payload mRNA) encoding an anti-CD19 CAR comprises a nucleic acid selected from the group consisting of SEQ ID NOS: 46-48, 441-458, 482-511, and 570-575. In some embodiments, the payload nucleic acid is a payload mRNA encoding an anti-CD19 CAR, wherein the payload mRNA comprises the RNA sequence of any one of SEQ ID NOs:570-575. In some embodiments, a nucleotide sequence encoding an anti-CD19 CAR further comprises a 5′ Cap. In some embodiments, the 5′ Cap is Cap 1. In some embodiments, a nucleotide sequence encoding an anti-CD19 CAR comprises Cap 1, a 5′ UTR comprising SEQ ID NO: 516, and a 3′ UTR comprises SEQ ID NO: 517. In some embodiments, a nucleotide sequence encoding an anti-CD19 CAR comprises Cap 1, a 5′ UTR comprising SEQ ID NO: 518, and the 3′ UTR comprises SEQ ID NO: 519. In some embodiments, a nucleotide sequence hybridizes to a nucleic acid comprising the complement of comprises a nucleic acid selected from the group consisting of SEQ ID NOs: 46-48, 441-458, and 482-511 under stringent conditions, such as: i) washing at 50° C. with 0.015 M NaCl, 0.0015 M sodium citrate and 0.1% SDS; or ii) washing at 42° C. in 0.2%×SSC and 0.1% SDS. In some embodiments, the payload comprises or is a nucleic acid selected from the group consisting of SEQ ID NOs: 46-48, 441-458, 482-511, and 570-575. In some embodiments, the payload comprises or is an mRNA selected from the group consisting of SEQ ID NOs:573-575.

In some embodiments, the payload RNA in the LNP is an in vitro transcribed (IVT) RNA. For example, the RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. In some embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. In some embodiments, 3′ UTRs can be selected or designed to increase the stability of the transcribed RNA. In some embodiments, the mRNA has both a cap on the 5′ end and a 3′ poly(A) tail (PolyA tail) which determine ribosome binding, initiation of translation and stability mRNA in the cell. The template DNA can be a circular DNA template or a linear DNA template. In some embodiments, poly (A) sequence is integrated into the RNA. In some embodiments, the poly (A) tail is arranged from about 100 nucleotides to between 100 and 200 nucleotides, between 200 and 300 nucleotides, or between 300 and 400 nucleotides. In some embodiments, the poly(A) tail comprises or is SEQ ID NO: 45.

In some embodiments, the RNA comprises one or more modified nucleosides. In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Kariko et ah, 2008, Mol Ther 16: 1833-1840; Anderson et ah, 2010, Nucleic Acids Res 38: 5884-5892; Anderson et al., 2011, Nucleic Acids Research 39: 9329-9338; Kariko et al., 2011, Nucleic Acids Research 39: el42; Kariko et al., 2012, Mol Ther 20: 948-953; Kariko et al., 2005, Immunity 23: 165-175). In some embodiments, the RNA comprises pseudouridines (e.g., 1-methyl-pseudouridines). In some embodiments, the modified nucleoside is mlacp,P (1-methyl-3-(3-amino-3-carboxypropyl) pseudouridine. In another embodiment, the modified nucleoside is mlxP (1-methylpseudouridine). In another embodiment, the modified nucleoside is *Pm (2′-O-methylpseudouridine). In another embodiment, the modified nucleoside is m5D (5-methyldihydrouridine). In another embodiment, the modified nucleoside is m3vP (3-methylpseudouridine). In another embodiment, the modified nucleoside is a pseudouridine moiety that is not further modified (*P). In another embodiment, the modified nucleoside is a monophosphate, diphosphate, or triphosphate of any of the above pseudouridines. In another embodiment, the modified nucleoside is any other pseudouridine-like nucleoside known in the art. Pseudouridines described herein can be incorporated into RNA molecules through methods known in the art, such as in vitro transcription.

In another embodiment, the nucleoside that is modified in the nucleoside-modified RNA is a modified uridine (U). In another embodiment, the modified nucleoside is a modified cytidine (C). In another embodiment, the modified nucleoside is a modified adenosine (A). In another embodiment, the modified nucleoside is a modified guanosine (G).

In another embodiment, the modified nucleoside of the present invention is m5C (5-methylcytidine). In another embodiment, the modified nucleoside is m5U (5-methyluridine). In another embodiment, the modified nucleoside is m6A (N6-methyladenosine). In another embodiment, the modified nucleoside is s2U (2-thiouridine). In another embodiment, the modified nucleoside is 'P (pseudouridine). In another embodiment, the modified nucleoside is Um (2′-O-methyluridine).

In other embodiments, the modified nucleoside is m1A (1-methyladenosine); m2A (2-methyladenosine); Am (2′-0-methyladenosine); ms2m6A (2-methylthio-N6-methyladenosine); i6A (N6-isopentenyladenosine); ms216A (2-methylthio-N6-isopentenyladenosine); io6A (N6-(cis-hydroxyisopentenyl) adenosine); ms2106A (2-methylthio-N6-(cis-hydroxyisopentenyl) adenosine); g6A (N6-glycinylcarbamoyladenosine); t6A (N6-threonylcarbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyladenosine); ms2hn6A (2-methylthio-N6-hydroxynorvalyl carbamoyladenosine); Ar(p) (2′-O-ribosyladenosine (phosphate)); I (inosine); ml! (1-methylinosine); m′lm (1,2′-O-dimethylinosine); m3C (3-methylcytidine); Cm (2′-O-methylcytidine); s2C (2-thiocytidine); ac4C (N4-acetylcytidine); PC (5-formylcytidine); m5Cm (5,2′-O-dimethylcytidine); ac4Cm (N4-acetyl-2′-O-methylcytidine); k2C (lysidine); m′G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr (p) (2′-O-ribosylguanosine (phosphate)); yW (wybutosine); 02yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQo (7-cyano-7-deazaguanosine); preQi (7-aminomethyl-7-deazaguanosme); G+(archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl) uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl) uridine)); mchm5U (5-(carboxyhydroxymethyl) uridine methyl ester); mcm5U (5-methoxy carbonylmethyluridine); mcm5Um (5-methoxycarbonylmethyl-2′-O-methyluridine); mcm5s2U (5-methoxycarbonylmethyl-2-thiouridine); nm5s2U (5-aminomethyl-2-thiouridine); mnm5U (5-methylaminomethyluridine); mnmVU (5-methylaminomethyl-2-thiouridine); mnm5se2U (5-methylaminomethyl-2-selenouridine); ncm5U (5-carbamoylmethyluridine); ncm5Um (5-carbamoylmethyl-2′-O-methyluridine); cmnm5U (5-carboxymethylaminomethyluridine); cmnm5Um (5-carboxymethylaminomethyl-21-O-methyluridine); cmnm5s2U (5-carboxymethylaminomethyl-2-thiouridine); m A (N6,N6-dimethyladenosine); Im (2′-O-methylinosine); m4C (N4-methylcytidine); m4Cm (N4,2′-O-dimethylcytidine); hm5C (5-hydroxymethylcytidine); m3U (3-methyluridine); cm5U (5-carboxymethyluridine); m6Am (N6,2′-O-dimethyladenosine); m62Am (N6,N6,0-2′-trimethyladenosine); m2 7G (N2,7-dimethylguanosine); m2 2 7G (N2,N2,7-trimethylguanosine); m3Um (3,2′-O-dimethyluridine); m5D (5-methyldihydrouridine); PCm (5-formyl-2′-O-methylcytidine); m′Gm (1,2′-O-dimethylguanosine); m′Am (1,2′-O-dimethyladenosine); Tm5U (5-taurinomethyluridine); rm5s2U (5-taurinomethyl-2-thiouridine)); imG-14 (4-demethylwyosine); imG2 (isowyosine); or ac6A (N6-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In various embodiments, between 0.1% and 100% of the residues in the nucleoside-modified of the present invention are modified (e.g., either by the presence of pseudouridine or another modified nucleoside base). In some embodiments, the fraction of modified residues is 0.1%. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%.

In some embodiments, the nucleic acid comprises pseudouridine. In some embodiments, the pseudouridine is N1-methyl-pseudouridine. In some embodiments, all uridines in an mRNA sequence of the present disclosure are replaced with N1-methyl-pseudouridine.

The LNP compositions may comprise an agent, for example, a nucleic acid molecule for delivery to a cell (e.g., an immune cell) or tissue, for example, a cell (e.g., an immune cell) or tissue in a subject.

The LNP compositions of the present invention may include a nucleic acid, for example, a DNA or RNA, such as an mRNA, tRNA, microRNA, siRNA, gRNA (guide RNA), circRNA (circular RNA), ribozymes, decoy RNA or dicer substrate siRNA. It is contemplated that nucleic acids can contain naturally occurring components, such as, naturally occurring bases, sugars or linkage groups (e.g., phosphodiester linkage groups) or may contain non-naturally occurring components or modifications, (e.g., thioester linkage groups). For example, the nucleic acid can be synthesized to contain base, sugar, linker modifications known to those skilled in the art. Furthermore, the nucleic acids can be linear or circular or can have any desired configuration. The LNP compositions can include multiple nucleic acid molecules, for example, multiple RNA molecules, which can be the same or different.

In certain embodiments, the payload is an mRNA. In certain embodiments, a particular LNP composition may contain a number of mRNA molecules that can be the same or different. In certain embodiments, one or more LNP compositions including one or more different mRNAs may be combined, and/or simultaneously contacted, with a cell. It is contemplated that an mRNA may include one or more of a stem loop, a chain terminating nucleoside, a poly A sequence, a polyadenylation signal, and/or a 5′ cap structure. The mRNA may encode a receptor, such as a chimeric antigen receptor (CAR), for use in for example, an immune disorder, inflammatory disorder or cancer. In addition, the mRNA may encode an antigen for use in a therapeutic or prophylactic vaccine, for example, for treating or preventing an infection by a pathogen, for example, a microbial or viral pathogen, or for reducing or ameliorating the side effects caused directly or indirectly by such an infection.

In certain embodiments, the LNP composition may include one or more other components including, but not limited to, one or more pharmaceutically acceptable excipients, small hydrophobic molecules, therapeutic agents, carbohydrates, polymers, permeability enhancing molecules, and surface altering agents.

In some embodiments, the wt/wt ratio of the lipid component to the payload (e.g., mRNA) in the resulting LNP composition is from about 1:1 to about 50:1. In certain embodiments, the wt/wt ratio of the lipid component to the payload (e.g., mRNA) in the resulting composition is from about 5:1 to about 50:1. In certain embodiments, the wt/wt ratio is from about 5:1 to about 40:1. In certain embodiments, the wt/wt ratio is from about 10:1 to about 40:1. In certain embodiments, the wt/wt ratio is from about 15:1 to about 25:1.

In certain embodiments, the encapsulation efficiency of the payload (e.g., mRNA) in the lipid nanoparticles is at least 50%. In certain embodiments, the encapsulation efficiency is at least 80%, at least 90% or, or greater than 90%.

In some embodiments, LNPs comprising an ionizable cationic lipid described herein, prepared and characterized using methods described herein, exhibit encapsulation efficiency of greater than 50, 55, 60, 65, 70, 75, 80, 82.5, 85, 87.5, 90, 92.5, 95, 97.5, or 99%. In some embodiments, LNPs comprising an ionizable cationic lipid described herein, prepared and characterized using methods described herein, exhibit encapsulation efficiency of greater than 87.5%. In some embodiments, LNPs comprising an ionizable cationic lipid described herein, prepared and characterized using methods described herein, exhibit dye accessible RNA of less than 50, 45, 40, 35, 30, 25, 20, 17.5, 15, 12.5, 10, 7.5, 5, 2.5, or 1%. In some embodiments, LNPs comprising an ionizable cationic lipid described herein, prepared and characterized using methods described herein, exhibit dye accessible RNA of less than 12.5%.

In some embodiments, LNPs comprising an ionizable cationic lipid described herein, prepared and characterized using methods described herein, exhibit total mRNA recovery of greater than 50, 55, 60, 65, 70, 75, 80, 85, 90, or 95%. In some embodiments, LNPs comprising an ionizable cationic lipid described herein, prepared and characterized using methods described herein, exhibit total mRNA recovery of greater than 80%.

In certain embodiments, the RNA payload is an mRNA, tRNA, microRNA, or siRNA payload.

In certain embodiments, the lipid nanoparticle compositions are optimized for the delivery of RNA, e.g., mRNA, to a target cell for translation within the cell. An mRNA may be a naturally or non-naturally occurring mRNA. An mRNA may include one or more modified nucleobases, nucleosides, or nucleotides.

The nucleobases may be selected from the non-limiting group consisting of adenine, guanine, uracil, cytosine, 7-methylguanine, 5-methylcytosine, 5-hydroxymethylcytosine, thymine, pseudouracil, dihydrouracil, N1-methylpseudouracil, hypoxanthine, and xanthine. In some embodiments, nucleobase is N1-methylpseudouracil.

A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications.

A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein.

An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. In certain embodiments, one or more or all uridine bases may be N1-methylpseudouridines.

In certain embodiments, an mRNA may include a 5′ cap structure, a chain terminating nucleotide, a stem loop, a poly A sequence, and/or a polyadenylation signal.

A cap structure or cap species is a compound including two nucleoside moieties joined by a linker and may be selected from a naturally occurring cap, a non-naturally occurring cap or a cap analog. A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G (5′) ppp (5′) G, commonly written as m7GpppG. A cap species may also be an anti-reverse cap analog. A non-limiting list of possible cap species includes m7GpppG, m7Gpppm7G, m73′dGpppG, m7Gpppm7G, m73′dGpppG, and m27 02′GppppG.

Alternatively or in addition, an mRNA may include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

Alternatively or in addition, an mRNA may include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a poly A sequence or tail.

Alternatively or in addition, an mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, the poly A sequence comprises about 50 to 200 adenine nucleotides, such as about 50, 60, 70, 80, 90, 100, 110, 120, 130, 140, 150, 160, 170, 180, 190, 200 adenine nucleotides. In some embodiments, the poly A sequence is SEQ ID NO: 45.

An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell. In some embodiments, the mRNA may encode an antibody, enzyme, growth factor, hormone, cytokine, viral protein (e.g., a viral capsid protein), antigen, vaccine, or receptor. In some embodiments, the mRNA may encode an engineered receptor such as a CAR or an antigen for use in a therapeutic vaccine (e.g., a cancer vaccine) or a prophylactic vaccine (e.g., a vaccine for minimizing the risk or severity of an infection by a microbial or viral pathogen). In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

A lipid composition may be designed for one or more specific applications or targets. For example, an LNP composition may be designed to deliver mRNA to a particular cell, tissue, organ, or system or group thereof in a mammal's body, such as the renal system. Physiochemical properties of LNP compositions may be altered in order to increase selectivity for particular target site within a subject. For instance, particle sizes may be adjusted based on the fenestration sizes of different organs. The mRNA included in an LNP composition may also depend on the desired delivery target or targets. For example, an mRNA may be selected for a particular indication, condition, disease, or disorder and/or for delivery to a particular cell, tissue, organ, or system or group thereof (e.g., localized or specific delivery).

The amount of mRNA in a lipid composition may depend on the size, sequence, and other characteristics of the mRNA. The amount of mRNA in an LNP may also depend on the size, composition, desired target, and other characteristics of the LNP composition. The relative amounts of mRNA and other elements (e.g., lipids) may also vary. The amount of mRNA in an LNP composition may, for example, be measured using absorption spectroscopy (e.g., ultraviolet-visible spectroscopy) or fluorescence spectroscopy (e.g., with detection dyes).

In some embodiments, the one or more mRNAs, lipids, and polymers and amounts thereof may be selected to provide a specific N:P ratio (the ratio of positively-chargeable lipid or polymer amine (N=nitrogen) groups to negatively-charged nucleic acid phosphate (P) groups). The N:P ratio of the composition refers to the molar ratio of nitrogen atoms in one or more lipids to the number of phosphate groups in an mRNA. In general, a lower N:P ratio is preferred. A N:P ratio may be dependent on a specific lipid and its pKa. In certain embodiments, the mRNA and LNP composition, and/or their relative amounts may be selected to provide an N:P ratio from about 1:1 to about 30:1, or from about 1:1 to about 20:1. In certain embodiments, the N:P ratio can be, for example, 1:1, 2:1, 3:1, 4:1, 5:1, 6:1, 7:1, or 8:1. In certain embodiments, the N:P ratio may be from about 2:1 to about 5:1. In certain embodiments, the N:P ratio may be about 4:1. In other embodiments, the N:P ratio is from about 4:1 to about 8:1. For example, the N:P ratio may be about 4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 6.0:1, about 6.5:1, or about 7.0:1.

The amount of mRNA in a nanoparticle composition may depend on the size, sequence, and other characteristics of the mRNA. The amount of mRNA in a nanoparticle composition may also depend on the size, composition, desired target, and other characteristics of the nanoparticle composition. The relative amounts of mRNA and other elements (e.g., lipids) may also vary. In some embodiments, the wt/wt ratio of the lipid component to an mRNA in a nanoparticle composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of the lipid component to an mRNA may be from about 10:1 to about 40:1. The amount of mRNA in a nanoparticle composition may, for example, be measured using absorption spectroscopy (e.g., ultraviolet-visible spectroscopy) or fluorescence spectroscopy (e.g., with detection dyes).

The efficiency of encapsulation of an mRNA describes the amount of mRNA that is encapsulated or otherwise associated with a lipid composition after preparation, relative to the initial amount provided. The encapsulation efficiency is desirably high (e.g., close to 100%). The encapsulation efficiency may be measured, for example, by comparing the amount of mRNA in a solution containing the lipid composition before and after breaking up the LNP composition with one or more organic solvents or detergents. Fluorescence may be used to measure the amount of free mRNA in a solution. For the LNP compositions of the present disclosure, the encapsulation efficiency of an mRNA may be at least 50%, for example 50%, 55%, 60%, 65%, 70%, 75%, 80%, 85%, 90%, 91%, 92%, 93%, 94%, 95%, 96%, 97%, 98%, 99%, or 100%. In certain embodiments, the encapsulation efficiency may be at least 80%.

### D. Other Lipid Components

In some embodiments, an LNP of the present disclosure comprises one or more structural lipids, one or more neutral phospholipids, or one or more free PEG-lipids, or any combination thereof.

### (i) Structural Lipid

In some embodiments, the structural lipid is sterol. In some embodiments, the structural lipid is cholesterol.

In certain embodiments, the LNP may comprise a sterol component, for example, one or more sterols selected from the group consisting of cholesterol, fecosterol, sitosterol, tomatidine, tomatine, ursolic acid, alpha-tocopherol, β-sitosterol, ergosterol, campesterol, stigmasterol, stigmastanol, brassicasterol, and mixtures thereof. In certain embodiments, the sterol is cholesterol. In some embodiments, the structural lipid includes cholesterol and a corticosteroid (such as prednisolone, dexamethasone, prednisone, and hydrocortisone), or a combination thereof.

The sterol (e.g., cholesterol) may be present in the LNP in a range of 20-70 mol %, 20-60 mol %, 20-50 mol %, 30-70 mol %, 30-60 mol %, 30-50 mol %, 40-70 mol %, 40-60 mol %, 40-50 mol %, 50-70 mol %, 50-60 mol %, or about 20 mol %, about 25 mol %, about 30 mol %, about 35 mol %, about 40 mol %, about 45 mol %, about 50 mol %, about 55 mol %, about 60 mol %, or about 65 mol %. In some embodiments, the sterol (e.g., cholesterol) may be present in the LNP in a range of about 25 mol % to about 45 mol %, such as about 30% to about 40%. In some embodiments, the sterol (e.g., cholesterol) may be present in the LNP about 28 mol % to 30 mol %, such as 29 mol % to about 30 mol % or such as about 29 mol %. In some embodiments, the sterol (e.g., cholesterol) may be present in the LNP about 38 mol % to 40 mol %, such as about 39 mol %. In some embodiments, the cholesterol is present in the LNP at a concentration of about 29 mol % when the DSPC is present in the LNP at a concentration of about 20 mol %. In some embodiments, the cholesterol is present in the LNP at a concentration of about 39 mol % when the DSPC is present in the LNP at a concentration of about 9.8 mol % or about 10 mol %.

### (ii) Neutral Phospholipid

In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dilinoleoyl-sn-glycero-3-phosphocholine (DLPC), 1,2-dimyristoyl-sn-glycero-phosphocholine (DMPC), 1,2-dipalmitoyl-sn-glycero-3-phosphocholine (DPPC), 1,2-diundecanoyl-sn-gly cero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₆ Lyso PC), 1,2-dilinolenoyl-sn-gly cero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some embodiments, the neutral phospholipid is DSPC.

In certain embodiments, the LNP may contain one or more neutral phospholipids. In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), and 1,2-dioleoyl-sn-glycero-3-phosphocholine (DOPC), sphingomyelin (SM).

Other neutral phospholipids can be selected from the group consisting of distearoyl-phosphatidylethanolamine (DSPE), dimyrstoyl-phosphatidylethanolamine (DMPE), distearoyl-glycero-phosphocholine (DSPC), hydrogenated soy phosphatidylcholine (HSPC), dioleoyl-glycero-phosphoethanolamine (DOPE), dilinoleoyl-glycero-phosphocholine (DLPC), dimyristoyl-glycero-phosphocholine (DMPC), dioleoyl-glycero-phosphocholine (DOPC), dipalmitoyl-glycero-phosphocholine (DPPC), diundecanoyl-glycero-phosphocholine (DUPC), palmitoyl-oleoyl-glycero-phosphocholine (POPC), dioctadecenyl-glycero-phosphocholine, oleoyl-cholesterylhemisuccinoyl-glycero-phosphocholine, hexadecyl-glycero-phosphocholine, dilinolenoyl-glycero-phosphocholine, diarachidonoyl-glycero-3-phosphocholine, didocosahexaenoyl-glycero-phosphocholine, and sphingomyelin.

In some embodiments, the neutral phospholipid (e.g., DSPC) is present in the LNP in a range of 1-30 mol %, 1-15 mol %, 1-12 mol %, 1-10 mol %, 3-15 mol %, 3-12 mol %, 3-10 mol %, 4-15 mol %, 4-12 mol %, 4-10 mol %, 4-8 mol %, 5-15 mol %, 5-12 mol %, 5-10 mol %, 6-15 mol %, 6-12 mol %, 6-10 more percent, or about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, or about 15 mol %, about 16 mol %, about 17 mol %, about 18 mol %, about 19 mol %, about 20 mol %, about 21 mol %, about 22 mol %, about 23 mol %, about 24 mol %, or about 15 mol %, about 26 mol %, about 27 mol %, about 28 mol %, about 29 mol %, about 30 mol %. In some embodiments, the neutral phospholipid (e.g., DSPC) is present in the LNP in a range of about 19 mol % to about 21 mol %, such as about 20 mol %.

(iii) Free PEG-Lipid

In some embodiments, the free PEG-lipid is a stabilizing lipid. In some embodiments, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. In some embodiments, the free PEG-lipid is PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, diacylphosphatidylethanolamine comprising Dipalmitoyl (C16) chain or Distearoyl (C18) chain, or a PEG-distearoyl-phosphatidylethanolamine (PEG-DSPE) lipid. In some embodiments, the PEG-DAG comprises PEG-DMG, PEG-DPG, or PEG-DSG, or any combination thereof. In some embodiments, the free PEG-lipid comprises PEG-DPG. In some embodiments, the PEG-DPG is PEG 2000-DPG.

In some embodiments, the LNP may include one or more PEG or PEG-modified lipids. Such species may be alternately referred to as PEGylated lipids. A PEG lipid is a lipid modified with polyethylene glycol. As noted above, free PEG-lipids can be included in the LNP to reduce or eliminate non-specific binding via a targeting group when a lipid-immune cell targeting group is included in the LNP.

A PEG lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

In certain embodiments, the blend may contain a free PEG-lipid that can be selected from the group consisting of PEG-distearoylglycerol (PEG-DSG), PEG-diacylglycerol (PEG-DAG, e.g., PEG-DMG, PEG-DPG, and PEG-DSG), PEG-dimyristoyl-glycerol (PEG-DMG), PEG-distearoyl-phosphatidylethanolamine (PEG-DSPE) and PEG-dimyrstoyl-phosphatidylethanolamine (PEG-DMPE). In some embodiments, the free PEG-lipid comprises a diacylphosphatidylcholines comprising Dipalmitoyl (C16) chain or Distearoyl (C18) chain.

The PEG-lipid may be present in the LNP in a range of 1-10 mol %, 1-8 mol %, 1-7 mol %, 1-6 mol %, 1-5 mol %, 1-4 mol %, 1-3 mol %, 2-8 mol %, 2-7 mol %, 2-6 mol %, 2-5 mol %, 2-4 mol %, 2-3 mol %, or about 1 mol %, about 2 mol %, about 3 mol %, about 4 mol %, or about 5 mol %. In some embodiments, the PEG-lipid is a free PEG-lipid.

In some embodiments, the PEG-lipid (e.g., PEG-DPG, such as PEG2.0K-DPG) may be present in the LNP in the range of 0.01-10 mol %, 0.01-5 mol %, 0.01-4 mol %, 0.01-3 mol %, 0.01-2 mol %, 0.01-1 mol %, 0.1-10 mol %, 0.1-5 mol %, 0.1-4 mol %, 0.1-3 mol %, 0.1-2 mol %, 0.1-1 mol %, 0.5-10 mol %, 0.5-5 mol %, 0.5-4 mol %, 0.5-3 mol %, 0.5-2 mol %, 0.5-1 mol %, 1-2 mol %, 3-4 mol %, 4-5 mol %, 5-6 mol %, or 1.25-1.75 mol %. In some embodiments, the PEG-lipid may be about 0.5 mol %, about 1 mol %, about 1.5 mol %, about 2 mol %, about 2.5 mol %, about 3 mol %, about 3.5 mol %, about 4 mol %, about 4.5 mol %, about 5 mol %, or about 5.5 mol % of the LNP. In some embodiments, the PEG-lipid (e.g., PEG-DPG, such as PEG2.0K-DPG) may be present in the LNP in about 1.2 to 1.8 mol %, such as about 1.5-1.6 mol %. In some embodiments, the PEG-lipid is a free PEG-lipid, such as PEG-DPG (e.g., PEG2.0K-DPG).

In some embodiments, the lipid anchor length of PEG-lipid is C14 (as in PEG-DMG). In some embodiments, the lipid anchor length of PEG-lipid is C16 (as in DPG). In some embodiments, the lipid anchor length of PEG-lipid is C18 (as in PEG-DSG). In some embodiments, the back-bone or head group of PEG-lipid is diacyl glycerol or phosphoethanolamine. In some embodiments, the PEG-lipid is a free PEG-lipid.

A LNP of the present disclosure may comprise one or more free PEG-lipid that is not conjugated to an immune cell targeting group, and a PEG-lipid that is conjugated to immune cell targeting group. In some embodiments, the free PEG-lipid comprises the same or a different lipid as the lipid in the lipid-immune cell targeting group conjugate.

### E. LNP Compositions

In some aspect, provided herein are LNP compositions. In some embodiments, the LNP composition comprises a lipid-immune cell targeting group conjugate, an ionizable cationic lipid, a payload, a structural lipid, a neutral phospholipid, or a free PEG-lipid, or any combination thereof. In some embodiments, the LNP composition comprise mRNA, Lipid 15, PEG 2000-DPG (same as DPG-PEG 2K), DSPC, cholesterol, and DSPE-PEG 3.4K-anti CD8 VHH conjugate. In some embodiments, the LNP composition comprises mRNA, Lipid 15, PEG 2000-DPG (same as DPG-PEG 2K), DSPC, cholesterol, and DSPE-PEG 3.4K-anti CD8 VHH conjugate. In some embodiments, the LNP is described in FIG. 50. In some embodiments, the LNP is described in the Table F-1 or Table F-2 below.

TABLE F-1
Exemplary LNP formulation (Mol percent, excluding payload content).

| | | Mol % | Mol % |
|---|---|---|---|
| | | in the LNP | in the LNP |
| | | or in the LNP | or in the LNP |
| | | composition | composition |
| Components | Description | (Formulation 1) | (Formulation 2) |
| Ionizable | (i) ionizable cationic Lipid 15 having the structure | about 48% to | about 48% to about |
| cationic lipid | below, | about 49.5% | 49.5% |
| | 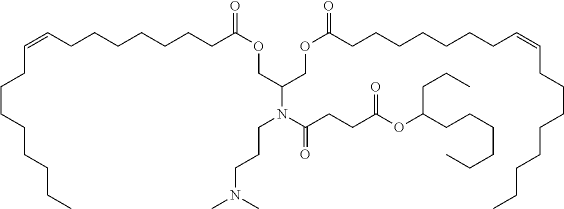 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC] | | |
| | Lipid 15 | | |
| | and/or a salt thereof; | | |
| Structural | (ii) cholesterol; | about 39% to | about 29% to about |
| lipid | | about 40% | 30% |
| Neutral lipid | (iii), DSPC; | about 9% to | about 19% to about |
| | | about 11% | 21% |
| Lipid-PEG | (iv) DPG-PEG; | about 1.4% to | about 1.4% to about |
| (PEG-lipid) | | about 1.6% | 1.6% |
| Targeting | (v) a DSPE-PEG3.4k-anti-CD8α antibody | about 0.01% to | about 0.01% to |
| moiety | conjugate, wherein the anti-CD8α antibody comprising | 0.02% | 0.02% |
| | SEQ ID NP: 9 SEQ ID NO: 169 or SEQ ID NO: 44. | | |

TABLE F-2
Exemplary LNP formulation (g/g mRNA)

| | | g/g mRNA | g/g mRNA |
|---|---|---|---|
| Components | Description | (Formulation 3) | (Formulation 4) |
| Ionizable cationic | (i) ionizable cationic Lipid 15 having the structure | about 14 to about | about 14 to about |
| lipid | below, | 15 | 15 |
| | 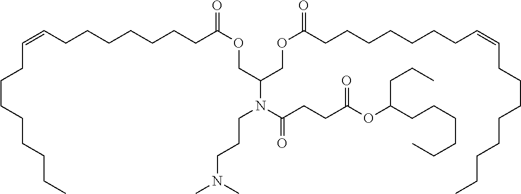 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC] | | |
| | Lipid 15 | | |
| | and/or a salt thereof; | | |
| Structural lipid | (ii) cholesterol; | about 4 to about 5 | about 3 to about 4 |
| Neutral lipid | (iii) DSPC; | about 2 to about 3 | about 4 to about 5 |
| Lipid-PEG | (iv) DPG-PEG; | about 1.0 to about | about 1.0 to about |
| (PEG-lipid) | | 1.5 | 1.5 |
| Targeting moiety | (v) a DSPE-PEG3.4k-anti-CD8α antibody conjugate, | about 0.05 to | about 0.05 to about |
| | wherein the anti-CD8α antibody comprising SEQ ID | about 0.10 | 0.10 |
| | NP: 9, SEQ ID NO: 169 or SEQ ID NO: 44. | | |

In some embodiments, an LNP as described in Table F-1 or Table F-2 herein comprises a payload mRNA comprising a nucleic acid sequence encoding a polypeptide comprising SEQ ID NO: 435, SEQ ID NO: 436, or SEQ ID NO: 437, wherein the mRNA comprises a 5′ cap (e.g., Cap 1), 5′ UTR, nucleic acids encoding an anti-CD19 CAR, 3′ UTR, and a poly (A) tail. In some embodiments, an LNP as described in Table F-1 or Table F-2 herein comprises a payload mRNA comprising a nucleic acid sequence encoding a polypeptide comprising SEQ ID NO: 49, SEQ ID NO: 50, or SEQ ID NO: 51, wherein the mRNA comprises a 5′ cap (e.g., Cap 1), 5′ UTR, nucleic acids encoding an anti-CD19 CAR, 3′ UTR, and a poly (A) tail.

### F. Additional Targeting Moieties

In some embodiments, the LNP further comprises one or more additional targeting moieties. In some embodiments, at least one additional targeting moiety is an anti-CD4 antibody (e.g., Ibalizumab). In some embodiments, the additional targeting moiety is in the same lipid-antibody conjugate for the anti-CD8 antibody, or in a different lipid-antibody conjugate. In some embodiments, the additional targeting moiety is in the same lipid-antibody conjugate for the anti-CD8 antibody, and the antibody in the conjugate is a bi-specific antibody targeting both CD8 and CD4. In some embodiments, the additional targeting moiety is in a different lipid-antibody conjugate. In some embodiments, the additional targeting moiety is an anti-CD4 antibody (e.g., Ibalizumab) in a different lipid-antibody conjugate. In some embodiments, the anti-CD8 conjugate and the anti-CD4 conjugate are presented in the LNP at a density of about 1.9 to 5.7 micromoles of conjugate per gram of mRNA in the LNP and about 1.9 to 11.4 micromoles of conjugate per gram of mRNA in the LNP respectively. In some embodiments, the anti-CD8 conjugate has a density of about 1.9 micromoles of conjugate per gram of mRNA in the LNP and the anti-CD4 conjugate has a density of about 11.4 micromoles of conjugate per gram of mRNA in the LNP.

### III. Methods of Making LNPs

a. Method of Producing LNPs

In some embodiments, the LNPs are produced by using either rapid mixing by an orbital vortexer or by microfluidic mixing. In some embodiments, the LNPs are produced by using T-mixing. Orbital vortexer mixing is accomplished by rapid addition of lipids solution in ethanol to the aqueous solution of a nucleic acid of interest followed immediately by vortexing at 2,500 rpm. In some embodiments, the LNPs are produced using a microfluidic mixing step. In some embodiments, microfluidic mixing is achieved mixing the aqueous and organic streams at a controlled flow rates in a microfluidic channel using, e.g., a NanoAssemblr device and microfluidic chips featuring optimized mixing chamber geometry (Precision Nanosystems, Vancouver, BC). In some embodiments, the LNPs are produced using a microfluidic mixing step to rapidly mix the ethanolic lipid solution and aqueous nucleic acid solution, resulting in encapsulation of the nucleic acid in the solid lipid nanoparticles. The nanoparticle suspension is then buffer exchanged into an all-aqueous buffer using membrane filtration device of choice for ethanol removal and nanoparticle maturation.

In certain embodiments, the resulting LNP compositions comprise, for example, from about 40 mol % to about 60 mol % of one or more ionizable cationic lipids described herein, from about 35 mol % to about 50 mol % of one or more sterols, from about 5 mol % to about 15 mol % of one or more neutral lipids, and from about 0.5 mol % to about 5 mol % of one or more PEG-lipids.

In certain embodiments, the resulting LNP compositions comprise, for example, from about 40 mol % to about 60 mol % of one or more ionizable cationic lipids described herein, from about 25 mol % to about 40 mol % of one or more sterols, from about 15 mol % to about 25 mol % of one or more neutral lipids, and from about 0.5 mol % to about 5 mol % of one or more PEG-lipids.

b. Physical Properties of LNPs

The characteristics of an LNP composition may depend on the components, their absolute or relative amounts, contained in a lipid nanoparticle (LNP) composition. Characteristics may also vary depending on the method and conditions of preparation of the LNP composition.

LNP compositions may be characterized by a variety of methods. For example, microscopy (e.g., transmission electron microscopy or scanning electron microscopy) may be used to examine the morphology and size distribution of an LNP composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of an LNP composition, such as particle size, polydispersity index, and zeta potential. RNA encapsulated efficiency is determined by a combination of methods relying on RNA binding dyes (ribogreen, cybergreen to determine dye accessible RNA fraction) and LNP de-formulation followed by HPLC analysis for total RNA content.

In some embodiments, the LNP may have a mean diameter in the range of 1-250 nm, 1-200 nm, 1-150 nm, 1-100 nm, 50-250 nm, 50-200 nm, 50-150 nm, 50-100 nm, 75-250 nm, 75-200 nm, 75-150 nm, 75-100 nm, 100-250 nm, 100-200 nm, 100-150 nm. In certain embodiments, the LNP compositions may have a mean diameter of about 1 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the LNP has a mean diameter of about 70 nm to about 100 nm.

In some embodiments, LNPs comprising an ionizable cationic lipid described herein, prepared and characterized using methods described herein, show average diameter change after a freeze-thaw of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change after a freeze-thaw of less than 30%. In some embodiments, the freeze-thaw and diameter measurements are conducted with 10% sucrose in MES pH 6.5 buffer.

In some embodiments, LNPs comprising an ionizable cationic lipid described herein, prepared and characterized using methods described herein, show average diameter change upon targeting antibody insertion of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change upon targeting antibody insertion of less than 15%. In some embodiments, the diameter change upon targeting antibody insertion is measured in pH 6.5 MES using a 37° C. incubation for 4 hours.

In some embodiments, LNPs comprising an ionizable cationic lipid described herein, prepared and characterized using methods described herein, have average LNP diameter of less than 50, 60, 70, 80, 90, 100, 110, 120, 130, 140, 150, 160, 170, 180, 190, or 200 nm. In some embodiments, LNPs comprising an ionizable cationic lipid described herein, prepared and characterized using methods described herein, have average LNP diameter of less than 100 nm.

Alternatively or in addition, the LNP compositions may have a polydispersity index in a range from 0.05-1, 0.05-0.75, 0.05-0.5, 0.05-0.4, 0.05-0.3, 0.05-0.2, 0.08-1, 0.08-0.75, 0.08-0.5, 0.08-0.4, 0.08-0.3, 0.08-0.2, 0.1-1, 0.1-0.75, 0.1-0.5, 0.1-0.4, 0.1-0.3, 0.1-0.2. In certain embodiments, the polydispersity index is in the range of 0.1-0.25, 0.1-0.2, 0.1-0.19, 0.1-0.18, 0.1-0.17, 0.1-0.16, or 0.1-0.15.

In some embodiments, the LNP compositions or LNPs comprising an ionizable cationic lipid described herein, prepared and characterized using methods described herein, have polydispersity of less than 0.4, 0.3, 0.25, 0.2, 0.15, 0.1, or 0.05. In some embodiments, LNPs comprising an ionizable cationic lipid described herein, prepared and characterized using methods described herein, have polydispersity of less than 0.25.

Alternatively or in addition, the LNP compositions may have a zeta potential of about −30 mV to about +30 mV. In certain embodiments, the LNP composition has a zeta potential of about −10 mV to about +20 mV. The zeta potential may vary as a function of pH. As a result, in certain embodiments, the LNP compositions may have a zeta potential of about 0 mV to about +30 mV or about +10 mV to +30 mV or about +20 mV to about +30 mV at pH 5.5 or pH 5, and/or a zeta potential of about −30 mV to about +5 mV or about −20 mV to about +15 mV at pH 7.4.

In some embodiments, the LNP compositions or LNPs comprising an ionizable cationic lipid described herein, prepared and characterized using methods described herein, have Zeta Potential at pH 7.4 greater than −15, −14, −13, −12, −11, −10, −9, −8, −7, −6, −5.5, −5, −4.5, −4, −3.5, −3, −2.5, −2, −1.5, −1, or −0.5 mV. In some embodiments, the LNP compositions or LNPs comprising an ionizable cationic lipid described herein, prepared and characterized using methods described herein, have Zeta Potential at pH 7.4 greater than −10 mV. In some embodiments, the LNP compositions or LNPs comprising an ionizable cationic lipid described herein, prepared and characterized using methods described herein, have Zeta Potential at pH 7.4 greater than −1 mV. In some embodiments, the LNP compositions or LNPs comprising an ionizable cationic lipid described herein, prepared and characterized using methods described herein, have Zeta Potential at pH 5.5 greater than −1, 0, 1, 2, 3, 4, 4.5, 5, 7.5, 10, 12.5, 15, 17.5, 20, 22.5, or 25 mV. In some embodiments, the LNP compositions or LNPs comprising an ionizable cationic lipid described herein, prepared and characterized using methods described herein, have Zeta Potential at pH 5.5 greater than 5 mV. In some embodiments, the LNP compositions or LNPs comprising an ionizable cationic lipid described herein, prepared and characterized using methods described herein, have Zeta Potential at pH 5.5 greater than 15 mV.

### IV. Methods of Using LNPs

The present disclosure provides methods of delivering a payload to a target cell or tissue, for example, a target cell or tissue in a subject, and LNPs or pharmaceutical compositions containing the LNPs for use in such methods. Any disclosure herein of a method of, e.g., treating a disease or disorder or, e.g., delivering a nucleic acid to a cell or, e.g., producing a polypeptide of interest in a cell should be interpreted also as a disclosure of an LNP or pharmaceutical composition comprising said LNP for use in such methods.

In certain embodiments, the present disclosure provides a method of producing a polypeptide of interest (e.g., a protein of interest) in a mammalian cell, and LNPs or pharmaceutical compositions containing the LNPs for use in such methods. Methods of producing polypeptides in such a cell involve contacting a cell with an LNP composition comprising an RNA of interest (e.g., an mRNA encoding the polypeptide of interest (e.g., a protein of interest). Upon contacting the cell with the LNP composition, the mRNA may be taken up and translated in the cell to produce the polypeptide of interest.

In certain embodiments, the present disclosure provides methods for expressing a payload mRNA in a target cell, tissue or organ in a subject, and mRNAs, LNPs or pharmaceutical compositions containing the mRNAs or LNPs for use in such methods. In some embodiments, the subject is diagnosed to have, expected to have, or expected to develop a lymphoma. In some embodiments, the lymphoma is a B cell lymphoma. In some embodiments, the B cell lymphoma is diffuse large B cell lymphoma (DLBCL). In some embodiments, the DLBCL is a relapse/refractory DLBCL. In some embodiments, the methods comprise contacting mRNAs, LNPs or pharmaceutical compositions containing the mRNA or LNPs described herein with a target cell, tissue, or organ of the subject.

In certain embodiments, the present disclosure provides methods for modifying gene expression in a target cell, issue or organ in a subject, and mRNAs, LNPs or pharmaceutical compositions containing the LNPs for use in such methods. In some embodiments, the methods comprise contacting a target cell, tissue, or organ in the subject with mRNAs, LNPs or pharmaceutical compositions containing the mRNA or LNPs described herein.

In certain embodiments, the present disclosure provides methods for modifying immune response in a subject, and mRNAs, LNPs or pharmaceutical compositions containing the mRNAs or LNPs for use in such methods. In some embodiments, the methods comprise administering the mRNAs, the LNPs or pharmaceutical compositions to the subject.

In certain embodiments, the present disclosure provides methods for activating T cells in a subject, and mRNAs, LNPs or pharmaceutical compositions containing the mRNAs or LNPs for use in such methods. In some embodiments, the methods comprise administering the mRNAs, the LNPs or the pharmaceutical compositions to the subject.

In certain embodiments, the present disclosure provides methods for inducing immune response against a cancerous cell in a subject, and mRNAs, LNPs or pharmaceutical compositions containing the mRNAs or LNPs for use in such methods. In some embodiments, the cancerous cell is a lymphoma cell. In some embodiments, the lymphoma cell is a B cell. In some embodiments, the methods comprise administering the mRNAs, the LNPs or the pharmaceutical compositions to the subject.

In certain embodiments, the present disclosure provides methods for treating B cell lymphoma in a subject, and mRNAs, LNPs or pharmaceutical compositions containing the mRNAs or LNPs for use in such methods. In some embodiments, the B cell lymphoma is diffuse large B cell lymphoma (DLBCL). In some embodiments, the DLBCL is a relapse/refractory DLBCL. In some embodiments, the methods comprise administering the mRNAs, the LNPs or the pharmaceutical composition to the subject.

In certain embodiments, the present disclosure provides methods for treating a B cell malignancy (e.g., a hematological cancer such as B-cell lymphoma, B-cell acute lymphoblastic leukemia (B-ALL), diffuse large B-cell lymphoma (DLBCL), follicular lymphoma, non-Hodgkin's lymphoma (NHL), or mantle cell lymphoma (MCL)) in a subject, and mRNAs, LNPs or pharmaceutical compositions containing the mRNAs or LNPs for use in such methods. In some embodiments, the B cell lymphoma is diffuse large B cell lymphoma (DLBCL). In some embodiments, the DLBCL is a relapse/refractory DLBCL. In some embodiments, the methods comprise administering the mRNAs, the LNPs or the pharmaceutical composition to the subject.

In certain embodiments, the present disclosure provides methods for treating an autoimmune disorder involving B cell activity in a subject, and mRNAs, LNPs or pharmaceutical compositions containing the mRNAs or LNPs for use in such methods. In some embodiments, the autoimmune disorder involving B cell activity is selected from the group consisting of: rheumatoid arthritis (RA), multiple sclerosis, type 1 diabetes mellitus (TID), primary Sjögren's syndrome (pSS), and systemic lupus erythematosus (SLE). In some embodiments, the methods comprise administering the mRNAs, the LNPs or the pharmaceutical composition to the subject.

In certain embodiments, the present disclosure provides methods for producing a modified T cell in vitro, ex vivo, or in vivo, and mRNAs, LNPs or pharmaceutical compositions containing the mRNAs or LNPs for use in such methods. In some embodiments, the methods comprise contacting the mRNAs, the LNPs or the pharmaceutical compositions to a T cell.

In certain embodiments, the present disclosure provides methods for producing a T cell with a Chimeric Antigen Receptor (CAR), and mRNAs, LNPs or pharmaceutical compositions containing the mRNAs or LNPs for use in such methods. In some embodiments, the methods comprise contacting the mRNAs, the LNPs or pharmaceutical compositions to a T cell.

In certain embodiments, the present disclosure provides methods for down-regulating B cell lymphoma activity in a subject, and mRNAs, LNPs or pharmaceutical compositions containing the mRNAs or LNPs for use in such methods. In some embodiments, the B cell lymphoma is diffuse large B cell lymphoma (DLBCL). In some embodiments, the DLBCL is a relapse/refractory DLBCL. In some embodiments, the methods comprise administering the mRNAs, the LNPs or the pharmaceutical compositions to the subject.

In certain embodiments, the present disclosure provides methods, wherein the methods comprise contacting a cell, tissue, or organ in a subject with mRNAs, LNPs or pharmaceutical compositions containing the mRNAs or LNPs as described herein.

In certain embodiments, the present disclosure provides methods, wherein the methods comprise administering a subject with mRNAs, LNPs or pharmaceutical compositions containing the mRNAs or LNPs as described herein.

In general, the step of contacting a mammalian cell with an mRNA or an LNP composition including an mRNA encoding a polypeptide of interest may be performed in vivo, ex vivo, or in vitro. The amount of the mRNA or the LNP composition contacted with a cell, and/or the amount of mRNA therein, may depend on the type of cell or tissue being contacted, the means of administration, the physiochemical characteristics of the LNP composition and the mRNA (e.g., size, charge, and chemical composition) therein, and other factors. In general, an effective amount of the mRNA or the LNP composition will allow for efficient polypeptide production in the cell. Metrics for efficiency may include polypeptide translation (indicated by polypeptide expression), level of mRNA degradation, and immune response indicators.

The step of contacting an mRNA or an LNP composition including an mRNA of the present disclosure with a cell may involve or cause transfection where the LNP composition or other vehicle for delivery the mRNA may fuse with the membrane of cell to permit the delivery of the mRNA into the cell. Upon introduction into the cytoplasm of the cell, the mRNA is then translated into a protein or peptide via the protein synthesis machinery within the cytoplasm of the cell.

In certain embodiments, the LNP compositions described herein may be used to deliver therapeutic or prophylactic agents to a subject. For example, an mRNA included in an LNP composition may encode a polypeptide and produce the therapeutic or prophylactic polypeptide upon contacting and/or entry (e.g., transfection) into a cell. In certain embodiments, an mRNA included in an LNP composition of the present disclosure may encode a polypeptide that may improve or increase the immunity of a subject.

In certain embodiments, contacting a cell with an mRNA or an LNP composition including an mRNA of the present disclosure may reduce the innate immune response of a cell to an exogenous nucleic acid. A cell may be contacted with a first exogenous mRNA or a first LNP composition including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times.

Additionally, efficiency of polypeptide production in the cell may be optionally determined, and the cell may be re-contacted with the first and/or second composition repeatedly until a target protein production efficiency is achieved.

The present disclosure provides methods of delivering a nucleic acid (e.g., an mRNA) to a mammalian cell or tissue, for example, a mammalian cell or tissue in a subject. Delivery of an mRNA to such a cell or tissue involves administering an LNP composition including the mRNA to a subject, for example, by injection, e.g., via intramuscular injection or intravascular delivery into the subject. After administration, the LNP can target and/or contact a cell, for example, an immune cell, such as a T cell. Upon contacting the cell with the LNP composition, a translatable mRNA may be translated in the cell to produce a polypeptide of interest.

In certain embodiments, an LNP composition of the present disclosure may target a particular type or class of cells. This targeting may be facilitated using the lipids described herein to form LNPs, which may also include a targeting group for targeting cells of interest. In certain, embodiments, specific delivery may result in a greater than 2 fold, 5 fold, 10 fold, 15 fold, or 20 fold increase in the amount of mRNA to the targeted destination (e.g., cells that express or express at high levels the receptor of interest which binds to the immune cell targeting group of the LNPs) as compared to another destinations (e.g., cells that either do not express or only express at low levels the receptor of interest).

mRNAs or LNP compositions comprising mRNAs of the present disclosure may be useful for treating a disease, disorder, or condition characterized by missing or aberrant protein or polypeptide activity. Upon delivery of an mRNA encoding the missing or aberrant polypeptide to a cell, translation of the mRNA may produce the polypeptide, thereby reducing or eliminating an issue caused by the absence of or aberrant activity caused by the polypeptide. Because translation may occur rapidly, the methods and compositions of the present disclosure may be useful in the treatment of acute diseases, disorders, or conditions such as sepsis, stroke, and myocardial infarction. An mRNA included in an LNP composition of the present disclosure may also be capable of altering the rate of transcription of a given species, thereby affecting gene expression.

Diseases, disorders, and/or conditions characterized by dysfunctional or aberrant protein or polypeptide activity for which a composition of the present disclosure may be administered include, but are not limited to, cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. The present disclosure provides a method for treating such diseases, disorders, and/or conditions in a subject by administering a composition including an mRNA as described herein (e.g., an LNP composition comprising the mRNA) to the subject.

The therapeutic and/or prophylactic compositions described herein may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject, the purpose of the administration, the particular composition, the mode of administration, and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

An mRNA or a LNP composition including one or more mRNAs may be administered by a variety of routes, for example, orally, intravenously, intramuscularly, intra-arterially, intramedullary, intrathecally, subcutaneously, intraventricularly, trans- or intra-dermally, intradermally, rectally, intravaginally, intraperitoneally, topically, mucosally, nasally, intratumorally. In certain embodiments, an mRNA composition (e.g., an LNP composition comprising the mRNA) may be administered intravenously, intramuscularly, intradermally, intra-arterially, intratumorally, or subcutaneously. However, the present disclosure encompasses the delivery of mRNA (e.g., LNP compositions comprising the mRNA) of the present disclosure by any appropriate route taking into consideration likely advances in the sciences of drug delivery. In general, the most appropriate route of administration will depend upon a variety of factors including the nature of the LNP composition including one or more mRNAs (e.g., its stability in various bodily environments such as the bloodstream and gastrointestinal tract), the condition of the patient (e.g., whether the patient is able to tolerate particular routes of administration), etc.

mRNAs or LNP compositions including one or more mRNAs of the present disclosure may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more LNP compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the present disclosure, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

In some embodiments, no more than 1%, no more than 2%, no more than 3%, no more than 4%, no more than 5%, no more than 6%, no more than 7%, no more than 8%, no more than 9%, no more than 10%, no more than 15%, no more than 20%, no more than 25%, no more than 30%, no more than 35%, no more than 40%, no more than 45%, or no more than 50% of cells that are not meant to be the destination of the delivery are transfected by the LNP. In some embodiments, the cells that are not meant to be the destination of the delivery are subject's non-immune cells. In some embodiments, the cells that are not meant to be the destination of the delivery are cells not targeted by the method. In some embodiments, the cells that are not meant to be the destination of the delivery are subject's cells not targeted by the method.

In some embodiments, the half-life of the nucleic acid delivered by the LNP described herein to the immune cell or a polypeptide encoded by the nucleic acid delivered by the LNP and expressed in the immune cell is at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, or at least 5 times longer than the half-life of the nucleic acid delivered by a reference LNP to the immune cells or a polypeptide encoded by the nucleic acid delivered by the reference LNP and expressed in the immune cell.

In some embodiments, the composition of the LNP differs from the composition of the reference LNP in the type of ionizable cationic lipid, relative amount of ionizable cationic lipid, length of the lipid anchor in PEG lipid, back bone or head group of the PEG lipid, relative amount of PEG lipid, or type of immune cell targeting group, or any combination thereof. In some embodiments, the composition of the LNP differs from the composition of the reference LNP only in the type of ionizable cationic lipid. In some embodiments, the composition of the LNP differs from the composition of the reference LNP only in the amount of PEG lipid. In some embodiments, the reference LNP comprises cationic Lipid DLin-KC3-DMA, but otherwise as the same as a tested LNP. In some embodiments, the reference LNP comprises cationic Lipid DLin-KC2-DMA, but otherwise as the same as a tested LNP. In some embodiments, the reference LNP comprises cationic Lipid ALC-0315, but otherwise as the same as a tested LNP. In some embodiments, the reference LNP comprises cationic Lipid SM-102, but otherwise as the same as a tested LNP. In some embodiments, PEG lipid is a free PEG lipid.

In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the immune cells are transfected by the LNP. In some embodiments, the immune cells are subject's immune cells. In some embodiments, the immune cells are immune cells targeted by the method. In some embodiments, the immune cells are subject's immune cells targeted by the method.

In some embodiments, the expression level of the nucleic acid delivered by the LNP is at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, at least 5 times, at least 6 times, at least 7 times, at least 8 times, at least 9 times, or at least 10 times higher than the expression level of the nucleic acid delivered by a reference LNP. In some embodiments, the expression level is measured and compared with a method described herein. In some embodiments, the expression level is measured by the ratio of cells expressing the encoded polypeptide. In some embodiments, the expression level is measured with FACS. In some embodiments, the expression level is measured by the average amount of the encoded polypeptide expressed in cells. In some embodiments, the expression level is measured as mean fluorescence intensity. In some embodiments, the expression level is measured by the amount of the encoded polypeptide or other materials secreted by cells.

In another aspect, provided herein are methods of targeting the delivery of a nucleic acid to an immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the compound of the following formula: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, an aspect of the disclosure relates to an LNP or a pharmaceutical composition containing thereof, as disclosed herein, for use in a method of targeting the delivery of a nucleic acid to an immune cell of a subject. Such a method may be for the treatment of a disease or disorder as disclosed hereafter. In some embodiments, a method as disclosed herein can comprise contacting in vivo, in vitro, or ex vivo the immune cell of a subject with a lipid nanoparticle (LNP). In some embodiments, the LNP is an LNP as described herein in the present disclosure.

In some embodiments, the LNP provides at least one of the following benefits:
(i) increased specificity of targeted delivery to the immune cell compared to a reference LNP;
(ii) increased half-life of the nucleic acid or a polypeptide encoded by the nucleic acid in the immune cell compared to a reference LNP;
(iii) increased transfection rate compared to a reference LNP; and
(iv) a low level of dye accessible mRNA (<15%) and high RNA encapsulation efficiencies, wherein at least 80% mRNA was recovered in final formulation relative to the total RNA used in LNP batch preparation.
In some aspect, provided are methods of expressing a polypeptide of interest in a targeted immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition containing thereof, as disclosed herein, for use in a method of expressing a polypeptide of interest in a targeted immune cell of a subject. Such a method may be for the treatment of a disease or disorder as disclosed hereafter. In some embodiments, a method as disclosed herein can comprise contacting in vivo, in vitro, or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP; and
(v) a low level of dye accessible mRNA (<15%) and high RNA encapsulation efficiencies, wherein at least 80% mRNA was recovered in final formulation relative to the total RNA used in LNP batch preparation.

In some aspects, provided are methods of modulating cellular function of a target immune cell of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the immune cell. In some embodiments, an aspect of the disclosure relates to an LNP or a pharmaceutical composition containing thereof, as disclosed herein, for use in a method of modulating cellular function of a targeted immune cell of a subject. Such a method may be for the treatment of a disease or disorder as disclosed hereafter. In some embodiments, a method as disclosed herein can comprise contacting in vivo, in vitro, or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP;
(v) the LNP can be administered at a lower dose compared to a reference LNP to reach the same biologic effect in the immune cell; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the modulation of cell function comprises reprogramming the immune cells to initiate an immune response. In some embodiments, the modulation of cell function comprises modulating antigen specificity of the immune cell.

In some aspect, provided are methods of treating, ameliorating, or preventing a symptom of a disorder or disease in a subject in need thereof. In some embodiments, the method comprises administering to the subject a lipid nanoparticle (LNP) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the nucleic acid modulates the immune response of the immune cell, therefore, to treat or ameliorate the symptom. In some embodiments, an aspect of the disclosure relates to an LNP or a pharmaceutical composition containing thereof, as disclosed herein, for use in a method of treating, ameliorating, or preventing a symptom of a disorder or disease in a subject in need thereof. A disease or disorder may be as disclosed hereafter. In some embodiments, a method as disclosed herein can comprise contacting in vivo, in vitro, or ex vivo the immune cell of a subject with a lipid nanoparticle (LNP).

In some embodiments, the LNP provides at least one of the following benefits:
(i) increased specificity of delivery of the nucleic acid into the immune cell compared to a reference LNP;
(ii) increased half-life of the nucleic acid or a polypeptide encoded by the nucleic acid in the immune cell compared to a reference LNP;
(iii) increased transfection rate compared to a reference LNP;
(iv) the LNP can be administered at a lower dose compared to a reference LNP to reach the same treatment efficacy;
(v) increased level of gain of function by an immune cell compared to a reference LNP; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the disorder is an immune disorder (e.g., an autoimmune disorder such as systemic lupus erythematosus, Sjögren's syndrome, and multiple sclerosis), an inflammatory disorder, or cancer. In some embodiments, the nucleic acid encodes an antigen for use in a therapeutic or prophylactic vaccine for treating or preventing an infection by a pathogen. In some embodiment, the disorder is lymphoma. In some embodiments, the lymphoma is a B cell lymphoma. In some embodiments, the B cell lymphoma is diffuse large B cell lymphoma (DLBCL). In some embodiments, the DLBCL is a relapse/refractory DLBCL.

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In some aspects, provided are methods of targeting the delivery of a nucleic acid to an immune cell of a subject. In some embodiments, the method comprises contacting the immune cell with a lipid nanoparticle (LNP) provided herein. In some embodiments, the method is for targeting T cells. In some embodiments, the immune cell targeting group binds to CD8α.

In some aspects, provided are methods of expressing a polypeptide of interest in a targeted immune cell of a subject. In some embodiments, the method comprises contacting the immune cell with an mRNA or a lipid nanoparticle (LNP) provided herein.

In some aspect, provided are method of modulating cellular function of a target immune cell of a subject. In some embodiments, the method comprises administering to the subject an mRNA or a lipid nanoparticle (LNP) provided herein.

In some aspects, provided are method of treating, ameliorating, or preventing a symptom of a disorder or disease in a subject in need thereof. In some embodiments, the subject is a subject in need thereof. In some embodiments, the method comprises administering to the subject an mRNA or a lipid nanoparticle (LNP) provided herein.

In some aspects, provided are methods of treating a disease or disorder related to CD8 in a subject. In some embodiments, the method comprises administering a pharmaceutical composition described herein to the subject. In some embodiments, the disease or disorder is cancer.

LNPs disclosed in the present disclosure and as claimed are suitable for the methods described above.

In some embodiments, when a substance described herein such as the LNP, isolated polynucleotide, expression construct, vector, host cell, ISVD, polypeptide, construct, and/or composition described herein is administered to a subject, it is administered in an effective amount, therapeutically effective amount, and/or prophylactically effective amount. In some embodiments, when a substance such as the LNP, isolated polynucleotide, expression construct, vector, host cell, ISVD, polypeptide, construct, and/or composition described herein is administered to a subject, it is administered in a pharmaceutically active amount.

### Formulation and Mode of Delivery

LNP compositions of the present disclosure may be formulated in whole or in part as a pharmaceutical composition. The pharmaceutical compositions may further include one or more pharmaceutically acceptable excipients or accessory ingredients such as those described herein. General guidelines for the formulation and manufacture of pharmaceutical compositions and agents are available, for example, in Remington's (2006) supra. Conventional excipients and accessory ingredients may be used in any pharmaceutical composition of the present disclosure, except insofar as any conventional excipient or accessory ingredient may be incompatible with one or more components of an LNP composition of the present disclosure. An excipient or accessory ingredient may be incompatible with a component of an LNP composition if its combination with the component may result in any undesirable biological effect or otherwise deleterious effect.

In some embodiments, one or more excipients or accessory ingredients may make up greater than 50% of the total mass or volume of a pharmaceutical composition including an LNP composition of the present disclosure. For example, the one or more excipients or accessory ingredients may make up 30%, 40%, 50%, 60%, 70%, 80%, 90%, or more of a pharmaceutical composition. In certain embodiments, the excipient is approved for use in humans and for veterinary use, for example, by United States Food and Drug Administration. In certain embodiments, the excipient is pharmaceutical grade. In certain embodiments, an excipient meets the standards of the United States Pharmacopoeia (USP), the European Pharmacopoeia (EP), the British Pharmacopoeia, and/or the International Pharmacopoeia.

Relative amounts of the one or more lipids or LNPs, one or more pharmaceutically acceptable excipients, and/or any additional ingredients in a pharmaceutical composition will vary, depending upon the identity, size, and/or condition of the subject treated and further depending upon the route by which the composition is to be administered.

Lipid compositions and/or pharmaceutical compositions including one or more LNP compositions may be administered to any subject, including a human patient that may benefit from a therapeutic effect provided by the delivery of a nucleic acid, e.g., an RNA (e.g., mRNA, tRNA or siRNA) to one or more particular cells, tissues, organs, or systems or groups thereof, such as the renal system. Although the descriptions provided herein of LNP compositions and pharmaceutical compositions including LNP compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is understood.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., the payload).

Pharmaceutical compositions of the present disclosure may be prepared in a variety of forms suitable for a variety of routes and methods of administration. For example, pharmaceutical compositions of the present disclosure may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

### V. Kits

Another aspect of the present disclosure provides a kit. In some embodiments, the kits are for treating a disorder. In some embodiments, the kit comprises an mRNA as described herein. In some embodiments, the kit comprises an LNP as described herein. In some embodiments, the kit comprises: an ionizable cationic lipid, a lipid-immune cell targeting group conjugate, a lipid nanoparticle composition comprising an ionizable cationic lipid and/or a lipid-immune cell targeting group conjugate with or without an encapsulated payload (e.g., an mRNA), and instructions for treating a medical disorder, such as, cancer or a microbial or viral infection.

### VI. Single Domain Antibodies

In another aspect, the disclosure provides anti-CD19 single domain antibodies (sdAbs or VHH antibodies). In some embodiments, the VHH comprises four framework regions (FR1 to FR4, respectively) and three complementarity determining regions (H-CDR1 to H-CDR3, respectively).

In some embodiments, the sdAb has an amino acid sequence selected from the group consisting of any one of SEQ ID Nos: 684-739, or an amino sequence comprising at least about 80% (such as at least about any of 85%, 86%, 87%, 88%, 89%, 90%, 91%, 92%, 93%, 94%, 95%, 96%, 97%, 98%, 99%, or more) sequence identity to the amino acid sequence selected from the group consisting of any one of SEQ ID Nos: 684-739.

In some embodiments, the VHH comprises an amino acid sequence of SEQ ID NO: 699. In some embodiments, the VHH comprises an HC-CDR1, an HC-CDR2, and an HC-CDR3 as set forth in SEQ ID NO: 699. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the VHH comprises an amino acid sequence of SEQ ID NO: 739. In some embodiments, the VHH comprises an HC-CDR1, an HC-CDR2, and an HC-CDR3 as set forth in SEQ ID NO: 739. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the VHH comprises an amino acid sequence of SEQ ID NO: 721. In some embodiments, the VHH comprises an HC-CDR1, an HC-CDR2, and an HC-CDR3 as set forth in SEQ ID NO: 721. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the VHH comprises an HC-CDR1, an HC-CDR2, and an HC-CDR3 according to the Abm definition, wherein VHH-CDR1 is or comprises the amino acid sequence depicted in SEQ ID and VHH-CDR3 is or comprises the amino acid sequence depicted in SEQ ID NO: 750.

In some embodiments, the VHH comprises an amino acid sequence of SEQ ID NO: 711. In some embodiments, the VHH comprises an HC-CDR1, an HC-CDR2, and an HC-CDR3 as set forth in SEQ ID NO: 711. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 850, VHH-CDR2 is or comprises the amino acid sequence depicted in SEQ ID NO: 851, and VHH-CDR3 is or comprises the amino acid sequence depicted in SEQ ID NO: 852. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 857, VHH-CDR2 is or comprises the amino acid sequence depicted in SEQ ID NO: 858, and VHH-CDR3 is or comprises the amino acid sequence depicted in SEQ ID NO: 859.

In some embodiments, the VHH comprises an amino acid sequence of SEQ ID NO: 723. In some embodiments, the VHH comprises an HC-CDR1, an HC-CDR2, and an HC-CDR3 as set forth in SEQ ID NO: 723. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the VHH comprises an amino acid sequence of SEQ ID NO: 724. In some embodiments, the VHH comprises an HC-CDR1, an HC-CDR2, and an HC-CDR3 as set forth in SEQ ID NO: 724. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID and VHH-CDR3 is or comprises the amino acid sequence depicted in SEQ ID NO: 749.

In some embodiments, the VHH comprises an amino acid sequence of SEQ ID NO: 725. In some embodiments, the VHH comprises an HC-CDR1, an HC-CDR2, and an HC-CDR3 as set forth in SEQ ID NO: 725. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the VHH comprises an amino acid sequence of SEQ ID NO: 726. In some embodiments, the VHH comprises an HC-CDR1, an HC-CDR2, and an HC-CDR3 as set forth in SEQ ID NO: 726. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the VHH comprises an amino acid sequence of SEQ ID NO: 727. In some embodiments, the VHH comprises an HC-CDR1, an HC-CDR2, and an HC-CDR3 as set forth in SEQ ID NO: 727. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID and VHH-CDR3 is or comprises the amino acid sequence depicted in SEQ ID NO: 749.

In some embodiments, the VHH comprises an amino acid sequence of SEQ ID NO: 728. In some embodiments, the VHH comprises an HC-CDR1, an HC-CDR2, and an HC-CDR3 as set forth in SEQ ID NO: 728. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the VHH comprises an amino acid sequence of SEQ ID NO: 729. In some embodiments, the VHH comprises an HC-CDR1, an HC-CDR2, and an HC-CDR3 as set forth in SEQ ID NO: 729. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the VHH comprises an amino acid sequence of SEQ ID NO: 730. In some embodiments, the VHH comprises an HC-CDR1, an HC-CDR2, and an HC-CDR3 as set forth in SEQ ID NO: 730. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID and VHH-CDR3 is or comprises the amino acid sequence depicted in SEQ ID NO: 749.

In some embodiments, the VHH comprises an amino acid sequence of SEQ ID NO: 731. In some embodiments, the VHH comprises an HC-CDR1, an HC-CDR2, and an HC-CDR3 as set forth in SEQ ID NO: 731. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the VHH comprises an amino acid sequence of SEQ ID NO: 732. In some embodiments, the VHH comprises an HC-CDR1, an HC-CDR2, and an HC-CDR3 as set forth in SEQ ID NO: 732. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the VHH comprises an amino acid sequence of SEQ ID NO: 733. In some embodiments, the VHH comprises an HC-CDR1, an HC-CDR2, and an HC-CDR3 as set forth in SEQ ID NO: 733. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 745. In some embodiments, the VHH comprises an HC-CDR1, an HC-CDR2, and an HC-CDR3 according to the Abm definition, wherein VHH-CDR1 is or comprises the amino acid sequence depicted in SEQ ID and VHH-CDR3 is or comprises the amino acid sequence depicted in SEQ ID NO: 751.

In some embodiments, the VHH comprises an amino acid sequence of SEQ ID NO: 734. In some embodiments, the VHH comprises an HC-CDR1, an HC-CDR2, and an HC-CDR3 as set forth in SEQ ID NO: 734. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 746. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 752.

In some embodiments, the VHH comprises an amino acid sequence of SEQ ID NO: 735. In some embodiments, the VHH comprises an HC-CDR1, an HC-CDR2, and an HC-CDR3 as set forth in SEQ ID NO: 735. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 743. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 749.

In some embodiments, the VHH comprises an amino acid sequence of SEQ ID NO: 736. In some embodiments, the VHH comprises an HC-CDR1, an HC-CDR2, and an HC-CDR3 as set forth in SEQ ID NO: 736. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 742, and VHH-CDR3 is or comprises the amino acid sequence depicted in SEQ ID NO: 744. In some embodiments, the VHH comprises an HC-CDR1, an HC-CDR2, and an HC-CDR3 according to the Abm definition, wherein VHH-CDR1 is or comprises the amino acid sequence depicted in SEQ ID and VHH-CDR3 is or comprises the amino acid sequence depicted in SEQ ID NO: 750.

In some embodiments, the VHH comprises an amino acid sequence of SEQ ID NO: 737. In some embodiments, the VHH comprises an HC-CDR1, an HC-CDR2, and an HC-CDR3 as set forth in SEQ ID NO: 737. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the VHH comprises an amino acid sequence of SEQ ID NO: 738. In some embodiments, the VHH comprises an HC-CDR1, an HC-CDR2, and an HC-CDR3 as set forth in SEQ ID NO: 738. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 741, and VHH-CDR3 is or comprises the amino acid sequence depicted in SEQ ID NO: 744. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 747, VHH-CDR2 is or comprises the amino acid sequence depicted in SEQ ID NO: 748, and VHH-CDR3 is or comprises the amino acid sequence depicted in SEQ ID NO: 750.

In some embodiments, the VHH comprises an amino acid sequence of SEQ ID NO: 715. In some embodiments, the VHH comprises an HC-CDR1, an HC-CDR2, and an HC-CDR3 as set forth in SEQ ID NO: 715. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 753, VHH-CDR2 is or comprises the amino acid sequence depicted in SEQ ID NO: 754, and VHH-CDR3 is or comprises the amino acid sequence depicted in SEQ ID NO: 755. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 756, VHH-CDR2 is or comprises the amino acid sequence depicted in SEQ ID NO: 757, and VHH-CDR3 is or comprises the amino acid sequence depicted in SEQ ID NO: 758.

In some embodiments, the VHH comprises an amino acid sequence of SEQ ID NO: 714. In some embodiments, the VHH comprises an HC-CDR1, an HC-CDR2, and an HC-CDR3 as set forth in SEQ ID NO: 714. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 759, VHH-CDR2 is or comprises the amino acid sequence depicted in SEQ ID NO: 760, and VHH-CDR3 is or comprises the amino acid sequence depicted in SEQ ID NO: 761. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 762, VHH-CDR2 is or comprises the amino acid sequence depicted in SEQ ID NO: 763, and VHH-CDR3 is or comprises the amino acid sequence depicted in SEQ ID NO: 764.

In some embodiments, the VHH comprises an amino acid sequence of SEQ ID NO: 707. In some embodiments, the VHH comprises an HC-CDR1, an HC-CDR2, and an HC-CDR3 as set forth in SEQ ID NO: 707. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 765, VHH-CDR2 is or comprises the amino acid sequence depicted in SEQ ID NO: 766, and VHH-CDR3 is or comprises the amino acid sequence depicted in SEQ ID NO: 767. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 768, VHH-CDR2 is or comprises the amino acid sequence depicted in SEQ ID NO: 769, and VHH-CDR3 is or comprises the amino acid sequence depicted in SEQ ID NO: 770.

In some embodiments, the VHH comprises an amino acid sequence of SEQ ID NO: 701. In some embodiments, the VHH comprises an HC-CDR1, an HC-CDR2, and an HC-CDR3 as set forth in SEQ ID NO: 701. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 771, VHH-CDR2 is or comprises the amino acid sequence depicted in SEQ ID NO: 772, and VHH-CDR3 is or comprises the amino acid sequence depicted in SEQ ID NO: 773. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 774, VHH-CDR2 is or comprises the amino acid sequence depicted in SEQ ID NO: 775, and VHH-CDR3 is or comprises the amino acid sequence depicted in SEQ ID NO: 776.

In some embodiments, the VHH comprises an amino acid sequence of SEQ ID NO: 718. In some embodiments, the VHH comprises an HC-CDR1, an HC-CDR2, and an HC-CDR3 as set forth in SEQ ID NO: 718. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 777, VHH-CDR2 is or comprises the amino acid sequence depicted in SEQ ID NO: 778, and VHH-CDR3 is or comprises the amino acid sequence depicted in SEQ ID NO: 779. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 780, VHH-CDR2 is or comprises the amino acid sequence depicted in SEQ ID NO: 781, and VHH-CDR3 is or comprises the amino acid sequence depicted in SEQ ID NO: 782.

In some embodiments, the VHH comprises an amino acid sequence of SEQ ID NO: 706. In some embodiments, the VHH comprises an HC-CDR1, an HC-CDR2, and an HC-CDR3 as set forth in SEQ ID NO: 706. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 783, VHH-CDR2 is or comprises the amino acid sequence depicted in SEQ ID NO: 784, and VHH-CDR3 is or comprises the amino acid sequence depicted in SEQ ID NO: 785. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 786, VHH-CDR2 is or comprises the amino acid sequence depicted in SEQ ID NO: 787, and VHH-CDR3 is or comprises the amino acid sequence depicted in SEQ ID NO: 788.

In some embodiments, the VHH comprises an amino acid sequence of SEQ ID NO: 687. In some embodiments, the VHH comprises an HC-CDR1, an HC-CDR2, and an HC-CDR3 as set forth in SEQ ID NO: 687. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 789, VHH-CDR2 is or comprises the amino acid sequence depicted in SEQ ID NO: 815, and VHH-CDR3 is or comprises the amino acid sequence depicted in SEQ ID NO: 816. In some embodiments, the VHH comprises an HC-CDR1, an HC-CDR2, and an HC-CDR3 according to the Abm definition, wherein VHH-CDR1 is or comprises the amino acid sequence depicted in SEQ ID NO: 817, VHH-CDR2 is or comprises the amino acid sequence depicted in SEQ ID NO: 818, and VHH-CDR3 is or comprises the amino acid sequence depicted in SEQ ID NO: 819.

In some embodiments, the VHH comprises an amino acid sequence of SEQ ID NO: 688. In some embodiments, the VHH comprises an HC-CDR1, an HC-CDR2, and an HC-CDR3 as set forth in SEQ ID NO: 688. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 841, VHH-CDR2 is or comprises the amino acid sequence depicted in SEQ ID NO: 842, and VHH-CDR3 is or comprises the amino acid sequence depicted in SEQ ID NO: 843.

In some embodiments, the VHH comprises an amino acid sequence of SEQ ID NO: 700. In some embodiments, the VHH comprises an HC-CDR1, an HC-CDR2, and an HC-CDR3 as set forth in SEQ ID NO: 700. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 844, VHH-CDR2 is or comprises the amino acid sequence depicted in SEQ ID NO: 845, and VHH-CDR3 is or comprises the amino acid sequence depicted in SEQ ID NO: 846.

In some embodiments, the VHH comprises an amino acid sequence of SEQ ID NO: 688. In some embodiments, the VHH comprises an HC-CDR1, an HC-CDR2, and an HC-CDR3 as set forth in SEQ ID NO: 688. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 841, VHH-CDR2 is or comprises the amino acid sequence depicted in SEQ ID NO: 842, and VHH-CDR3 is or comprises the amino acid sequence depicted in SEQ ID NO: 843.

In some embodiments, the VHH comprises an amino acid sequence of SEQ ID NO: 710. In some embodiments, the VHH comprises an HC-CDR1, an HC-CDR2, and an HC-CDR3 as set forth in SEQ ID NO: 710. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 847, VHH-CDR2 is or comprises the amino acid sequence depicted in SEQ ID NO: 848, and VHH-CDR3 is or comprises the amino acid sequence depicted in SEQ ID NO: 849.

In some embodiments, the VHH comprises an amino acid sequence of SEQ ID NO: 713. In some embodiments, the VHH comprises an HC-CDR1, an HC-CDR2, and an HC-CDR3 as set forth in SEQ ID NO: 713. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 853, VHH-CDR2 is or comprises the amino acid sequence depicted in SEQ ID NO: 854, and VHH-CDR3 is or comprises the amino acid sequence depicted in SEQ ID NO: 429.

In some embodiments, the VHH comprises an amino acid sequence of SEQ ID NO: 719. In some embodiments, the VHH comprises an HC-CDR1, an HC-CDR2, and an HC-CDR3 as set forth in SEQ ID NO: 719. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 740, VHH-CDR2 is or comprises the amino acid sequence depicted in SEQ ID NO: 855, and VHH-CDR3 is or comprises the amino acid sequence depicted in SEQ ID NO: 856.

In some embodiments, the VHH comprises an amino acid sequence of SEQ ID NO: 717. In some embodiments, the VHH comprises an HC-CDR1, an HC-CDR2, and an HC-CDR3 as set forth in SEQ ID NO: 717. In some embodiments, the VHH comprises an HC-CDR1, an HC-CDR2, and an HC-CDR3 according to the Kabat definition, wherein VHH-CDR1 is or comprises the amino acid sequence depicted in SEQ ID NO: 753, VHH-CDR2 is or comprises the amino acid sequence depicted in SEQ ID NO: 754, and VHH-CDR3 is or comprises the amino acid sequence depicted in SEQ ID NO: 755.

In one embodiment, the VHH comprises four framework regions (FR1 to FR4, respectively) and three complementarity determining regions (H-CDR1 to H-CDR3, respectively), wherein:
H-CDR1 (Kabat definition) consists of an amino acid sequence selected from:
   j) the amino acid sequence of SYAMA (SEQ ID NO: 740);
   k) amino acid sequences that have at least 80% amino acid identity with the amino acid sequence of SYAMA (SEQ ID NO: 740); and
   l) amino acid sequences that have 3, 2, or 1 amino acid difference with the amino acid sequence of SYAMA (SEQ ID NO: 740);
and
H-CDR2 (Kabat definition) consists of an amino acid sequence selected from:
   m) the amino acid sequence of VISLSGGSVDYX₂DSVKG (SEQ ID NO: 839), wherein X₂ is R or A;
   n) amino acid sequences that have at least 80% amino acid identity with the amino acid sequence of VISLSGGSVDYX₂DSVKG (SEQ ID NO: 839), wherein X₂ is R or A; and
   o) amino acid sequences that have 3, 2, or 1 amino acid difference with the amino acid sequence of VISLSGGSVDYX2DSVKG (SEQ ID NO: 839), wherein X₂ is R or A;
and
H-CDR3 (Kabat definition) consists of an amino acid sequence selected from:
   p) the amino acid sequence of RPVYX₁KWSERDFDY (SEQ ID NO: 815), wherein the amino acid residue X₁ is selected from M, I, L and V;
   q) amino acid sequences that have at least 80% amino acid identity with the amino acid sequence of RPVYX₁KWSERDFDY (SEQ ID NO: 815), wherein the amino acid residue X₁ is selected from M, I, L and V; and
   r) amino acid sequences that have 3, 2, or 1 amino acid difference with the amino acid sequence of RPVYX₁KWSERDFDY (SEQ ID NO: 815), wherein the amino acid residue X₁ is selected from M, I, L and V.

In some embodiments, FR1 comprises an amino acid sequence (as defined by Kabat numbering) selected from the group consisting of SEQ ID NO: 820, SEQ ID NO: 834, and SEQ ID NO: 835, or amino acid sequences that have at least 80% amino acid identity with the amino acid sequence (as defined by Kabat numbering) selected from the group consisting of SEQ ID NO: 820, SEQ ID NO: 834, and SEQ ID NO: 835.

In some embodiments, FR1 comprises an amino acid sequence (as defined by Abm numbering) selected from the group consisting of SEQ ID NO: 792, SEQ ID NO: 836, and SEQ ID NO: 837, or amino acid sequences that have at least 80% amino acid identity with the amino acid sequence (as defined by Abm numbering) selected from the group consisting of SEQ ID NO: 792, SEQ ID NO: 836, and SEQ ID NO: 837.

In some embodiments, FR2 comprises an amino acid sequence (as defined by Kabat numbering) selected from the group consisting of SEQ ID NO: 821, SEQ ID NO: 822, and SEQ ID NO: 823, or amino acid sequences that have at least 80% amino acid identity with the amino acid sequence (as defined by Kabat numbering) selected from the group consisting of SEQ ID NO: 821, SEQ ID NO: 822, and SEQ ID NO: 823.

In some embodiments, FR2 comprises an amino acid sequence (as defined by Abm numbering) selected from the group consisting of SEQ ID NO: 824, SEQ ID NO: 825, and SEQ ID NO: 826, or amino acid sequences that have at least 80% amino acid identity with the amino acid sequence (as defined by Abm numbering) selected from the group consisting of SEQ ID NO: 824, SEQ ID NO: 825, and SEQ ID NO: 826.

In some embodiments, FR3 comprises an amino acid sequence (as defined by Kabat numbering) selected from the group consisting of SEQ ID NO: 827, SEQ ID NO: 790, SEQ ID NO: 791, SEQ ID NO: 797, SEQ ID NO: 798, SEQ ID NO: 799, SEQ ID NO: 800, SEQ ID NO: 801, SEQ ID NO: 802, and SEQ ID NO: 803, or amino acid sequences that have at least 80% amino acid identity with the amino acid sequence (as defined by Kabat numbering) selected from the group consisting of SEQ ID NO: 827, SEQ ID NO: 790, SEQ ID NO: 791, SEQ ID NO: 797, SEQ ID NO: 798, SEQ ID NO: 799, SEQ ID NO: 800, SEQ ID NO: 801, SEQ ID NO: 802, and SEQ ID NO: 803.

In some embodiments, FR3 comprises an amino acid sequence (as defined by Abm numbering) selected from the group consisting of SEQ ID NO: 793, SEQ ID NO: 794, SEQ ID NO: 795, SEQ ID NO: 804, SEQ ID NO: 805, SEQ ID NO: 806, SEQ ID NO: 807, SEQ ID NO: 808, SEQ ID NO: 809, SEQ ID NO: 810, SEQ ID NO: 811, SEQ ID NO: 812, SEQ ID NO: 813, and SEQ ID NO: 814, or amino acid sequences that have at least 80% amino acid identity with the amino acid sequence (as defined by Abm numbering) selected from the group consisting of SEQ ID NO: 793, SEQ ID NO: 794, SEQ ID NO: 795, SEQ ID NO: 804, SEQ ID NO: 805, SEQ ID NO: 806, SEQ ID NO: 807, SEQ ID NO: 808, SEQ ID NO: 809, SEQ ID NO: 810, SEQ ID NO: 811, SEQ ID NO: 812, SEQ ID NO: 813, and SEQ ID NO: 814

In some embodiments, FR4 comprises an amino acid sequence (as defined by Kabat numbering) selected from the group consisting of SEQ ID NO: 828, SEQ ID NO: 829, and SEQ ID NO: 830, or amino acid sequences that have at least 80% amino acid identity with the amino acid sequence (as defined by Kabat numbering) selected from the group consisting of SEQ ID NO: 831, SEQ ID NO: 832, and SEQ ID NO: 833.

In some embodiments, FR4 comprises an amino acid sequence (as defined by Abm numbering) selected from the group consisting of SEQ ID NO: 792, SEQ ID NO: 836, and SEQ ID NO: 837, or amino acid sequences that have at least 80% amino acid identity with the amino acid sequence (as defined by Abm numbering) selected from the group consisting of SEQ ID NO: 792, SEQ ID NO: 836, and SEQ ID NO: 837.

In some embodiments, the disclosure provides an anti-CD19 antigen-binding domain, wherein the anti-CD19 antigen-binding domain is an anti-CD19 single domain antibody (anti-CD19 sdAb) domain comprising an anti-CD19 VHH comprising: i) an H-CDR1 comprising the amino acid sequence (Kabat numbering) of SEQ ID NO: 850, an H-CDR2 comprising the amino acid sequence of SEQ ID NO: 851 (Kabat numbering), and an H-CDR3 comprising the amino acid sequence of SEQ ID NO: 852 (Kabat numbering), or (ii) an H-CDR1 comprising the amino acid sequence of SEQ ID NO: 857 (Abm numbering), an H-CDR2 comprising the amino acid sequence of SEQ ID NO: 858 (Kabat numbering), and an H-CDR3 comprising the amino acid sequence of SEQ ID NO: 859 (Kabat numbering).

### EXAMPLES

The invention now being generally described, will be more readily understood by reference to the following examples, which are included merely for purposes of illustration of certain aspects and embodiments of the present invention, and are not intended to limit the invention.

### Example 1. Preparation of Ionizable Cationic Lipids

This Example describes the synthesis of various cationic lipids.

### General Scheme for the Synthesis of Lipids 1 Through Lipid 30

A general scheme for the synthesis of Lipids 1 through Lipid 30 is provided in Scheme 1 below. Corresponding R and R′ for each lipid is provided in Tables 2 to 4 below.

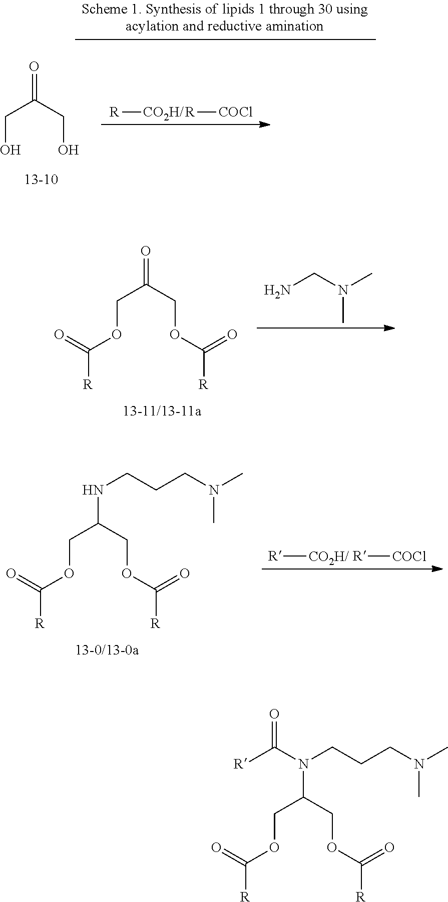

### Synthesis of Intermediates 13-11 and 13-11a

Intermediate 13-11 (Scheme 2) was synthesized by acylation of dihydroxyacetone (13-10) with linoleic acid. Dihydroxyacetone (22 mmol, 2 g, 1 eq.) was reacted with linoleic acid 1-5 (55 mmol, 15.4 g, 2.5 eq.) using EDCI (55 mmol, 10.5 g, 2.5 eq.) activation in 50 mL DCM, in the presence of DIPEA (55 mmol, 9.6 mL, 2.5 eq.), DMAP (4.4 mmol, 540 mg, 0.2 eq.) at room temperature yielding 11.1 g (79%) crude product. Purified product was obtained by column chromatography and characterized by proton NMR spectroscopy (FIG. 1).

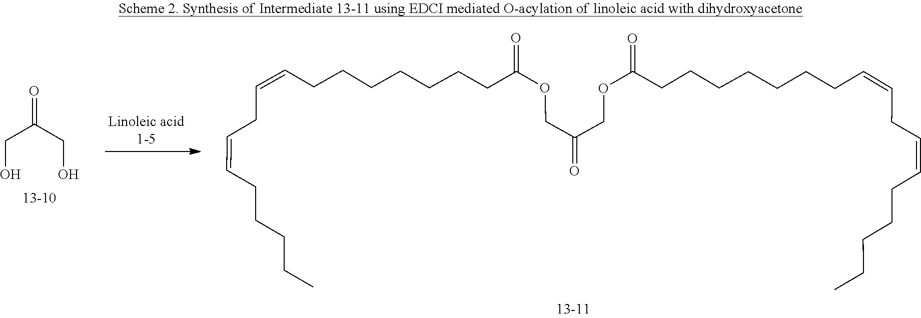 [SMILES: O=C(CO)CO ; CCCCC/C=C\C/C=C\CCCCCCCC(=O)OCC(=O)COC(=O)CCCCCCC/C=C\C/C=C\CCCCC]

Intermediates 13-11a (Scheme 3) was synthesized by acylation of dihydroxyacetone (13-10) with oleoyl chloride. Dihydroxyacetone (44.4 mmol, 4 g, 1 eq.) was reacted with oleoyl chloride 1-6a (111 mmol, 36.7 mL, 2.5 eq.) in the presence of Pyridine (133.3 mmol, 11 mL, 3 eq.), DMAP (13.3 mmol, 1.63 g, 0.3 eq.) in 80 mL DCM, at room temperature yielding 14.9 g (54%) crude product. Crude product was purified by column chromatography and characterized by proton NMR spectroscopy (FIG. 2A).

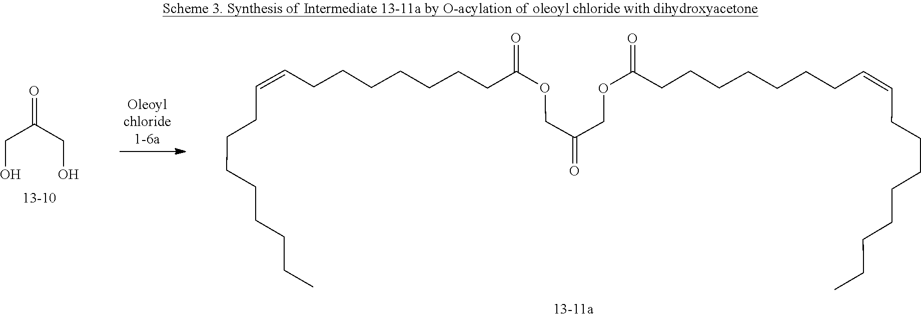 [SMILES: O=C(CO)CO ; CCCCCCCC/C=C\CCCCCCCC(=O)OCC(=O)COC(=O)CCCCCCC/C=C\CCCCCCCC]

### Synthesis of Intermediates 13-0a and 13-11b

Intermediates 13-0a and 13-11b were synthesized by reductive amination of intermediates 13-11 and 13-11a respectively.

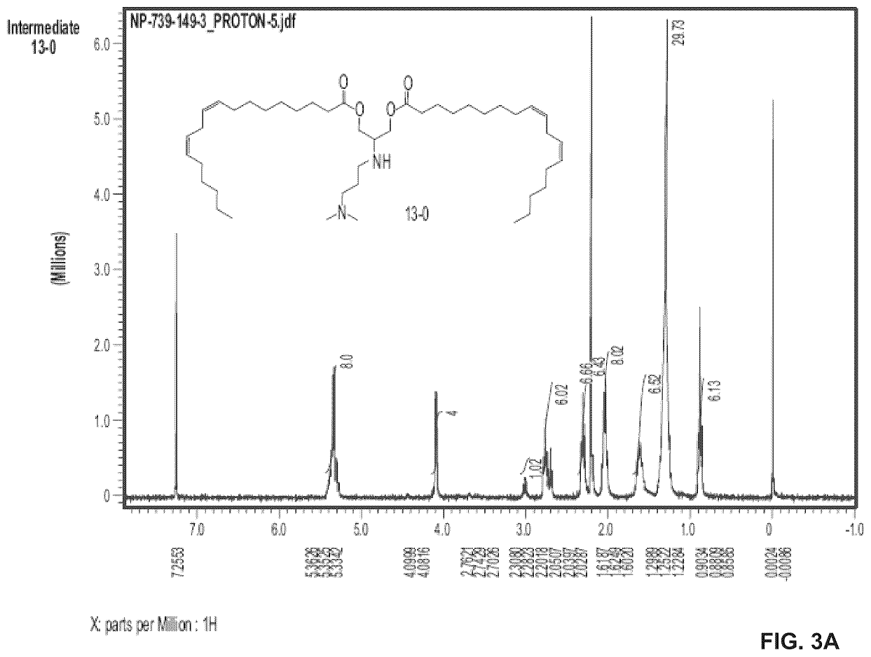

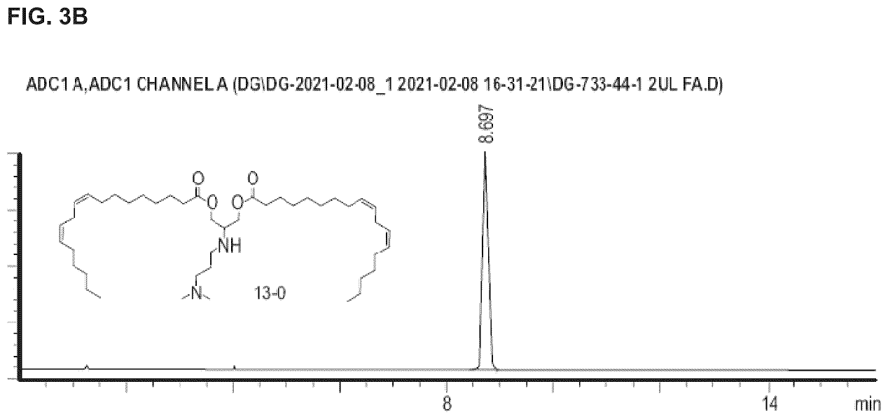

Intermediate 13-0 was produced by reductive amination (scheme 4) of intermediate 13-11 (13.1 mmol, 8.1 g, 1.0 eq) using N1,N1-dimethylpropane-1,3-diamine 15-3 (26 mmol, 3.2 mL, 2.0 eq.) in DCM (10 mL) using acetic acid (26.0 mmol, 1.50 mL, 2 eq.) and sodium borohydride triacetate (4.32 mmol, 3.3 g, 1.2 eq.) yielding 3.1 g (32%) crude product. Column purification resulted in pure product (Proton NMR Spectrum and LC-CAD chromatogram shown in FIG. 3A and FIG. 3B, respectively).

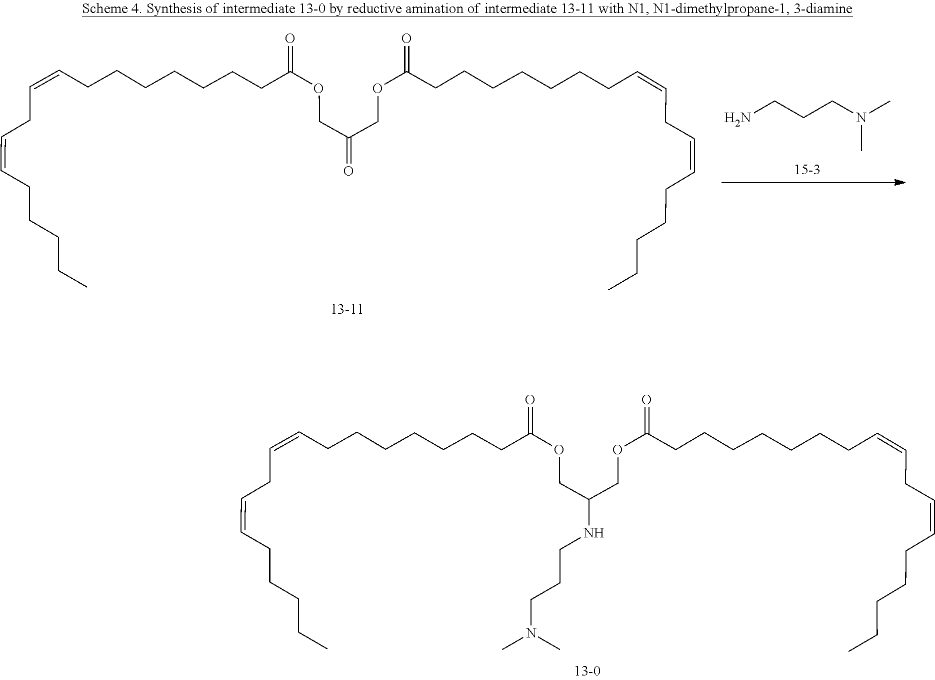

Intermediate 13-11b was produced by reductive amination (scheme 5) of intermediate 13-11a (24.2 mmol, 14.9 g, 1.0 eq) using N1,N1-dimethylpropane-1,3-diamine 15-3 (48.4 mmol, 6.05 mL, 2.0 eq.) in DCM (60 mL) using acetic acid (48.4 mmol, 2.8 mL, 2 eq.) and sodium borohydride triacetate (29.1 mmol, 6.05 g, 1.2 eq.) yielding 6 g (35%) crude product. Column purification resulted in purified product (Proton NMR Spectrum and LC-ELSD chromatogram shown in FIG. 2B and FIG. 2C, respectively).

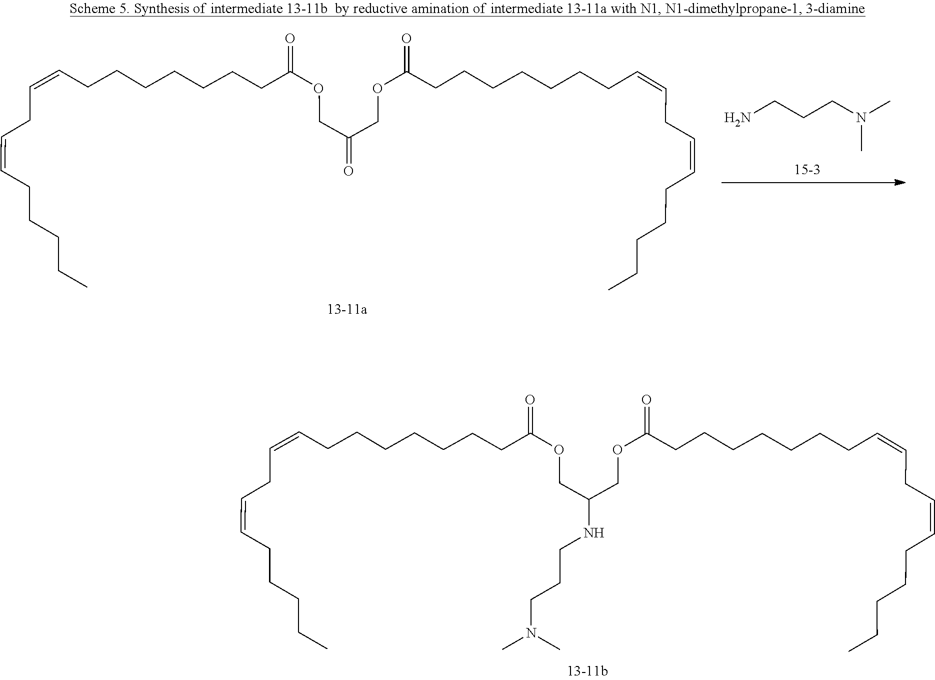

TABLE 2
R (O-acyl) and R′ (N-acyl) groups of lipids 1 through 8.

| Lipid # | R (O-acyl groups) | R′ (N-acyl group) |
|---|---|---|
| Lipid 1 | 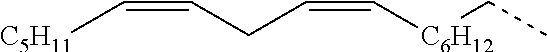 | 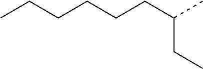 |
| Lipid 2 | 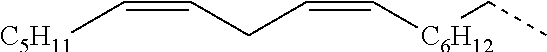 | 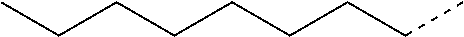 |
| Lipid 3 | 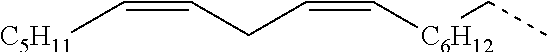 | 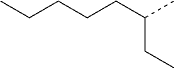 |
| Lipid 4 | 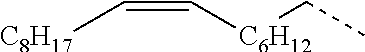 | 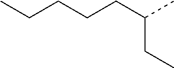 |
| Lipid 5 | 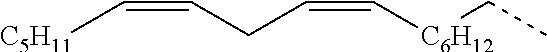 | 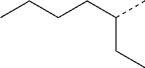 |
| Lipid 6 | 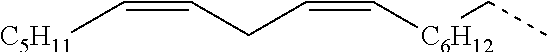 | 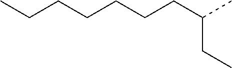 |
| Lipid 7 | 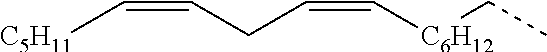 | 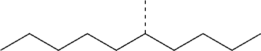 |
| Lipid 8 | 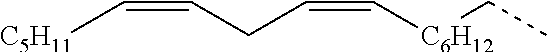 | 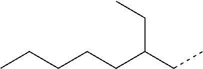 |

TABLE 3
R (O-acyl) and R′ (N-acyl) groups of Lipids 9 through 16.

| Lipid # | R (O-acyl groups) | R′ (N-acyl group) |
|---|---|---|
| Lipid 9 | 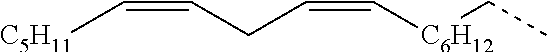 | 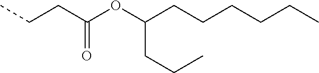 |
| Lipid 10 | 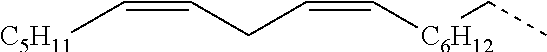 | 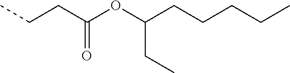 |
| Lipid 10A | 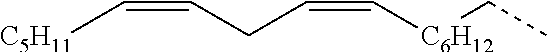 | 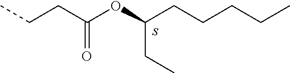 |
| Lipid 11 | 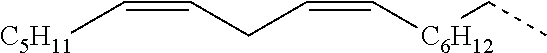 | 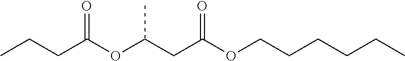 [SMILES: CCCCCCOC(=O)C[C@@H](C)OC(=O)CCC] |
| Lipid 11A | 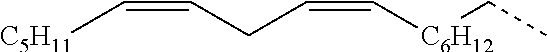 | 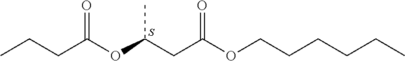 |
| Lipid 12 | 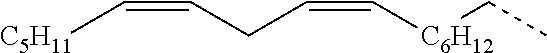 | 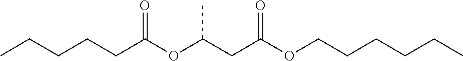 [SMILES: CCCCCCOC(=O)C[C@@H](C)OC(=O)CCCCC] |
| Lipid 13 | 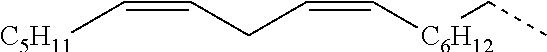 | 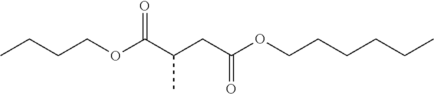 [SMILES: CCCCCCOC(=O)C[C@H](C)C(=O)OCCCC] |
| Lipid 14 | 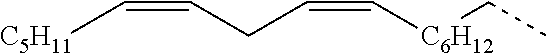 | 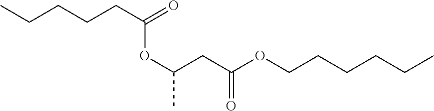 [SMILES: CCCCCCOC(=O)C[C@H](C)OC(=O)CCCCC] |
| Lipid 14A | 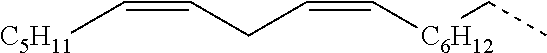 | 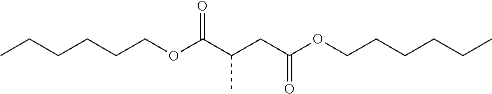 [SMILES: CCCCCCOC(=O)C[C@H](C)C(=O)OCCCCCC] |
| Lipid 15 | 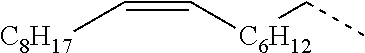 | 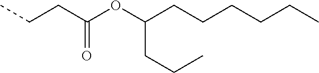 |
| Lipid 16 | 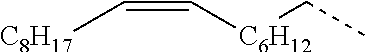 | 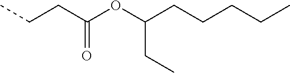 |

TABLE 4-1
R (O-acyl) and R′ (N-acyl) groups of Lipids 17, 17A, 18, 18A, 19, 19A, 20, 20A,
21, 21A, 22, 23, and 23A.

| Lipid # | R (O-acyl groups) | R′ (N-acyl group) |
|---|---|---|
| Lipid 17 | 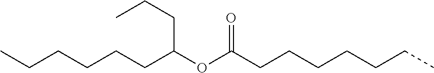 | 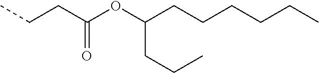 |
| Lipid 17A | 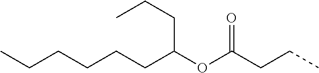 | 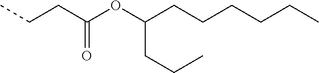 |
| Lipid 18 | 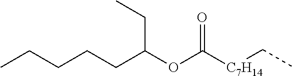 | 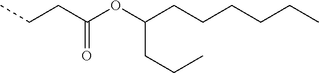 |
| Lipid 18A | 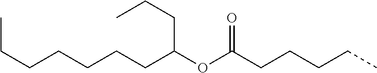 | 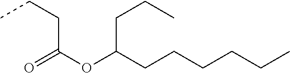 |
| Lipid 19 | 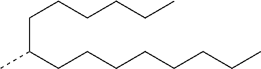 | 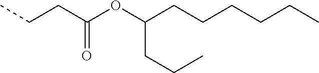 |
| Lipid 19A | 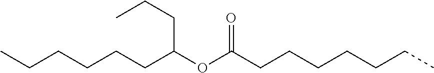 | 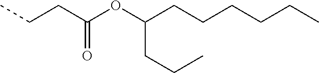 |
| Lipid 20 | 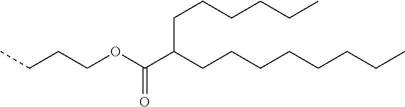 | 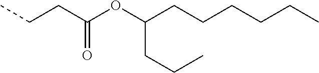 |
| Lipid 20A | 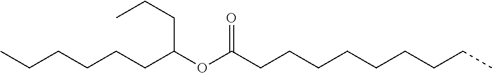 | 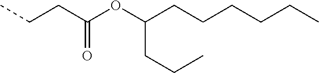 |
| Lipid 21 | 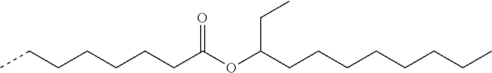 | 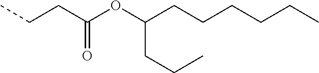 |
| Lipid 21A | 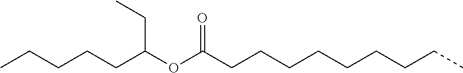 | 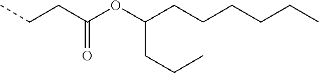 |
| Lipid 22 | 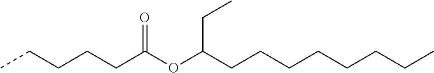 | 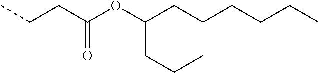 |
| Lipid 23 | 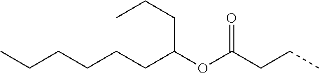 | 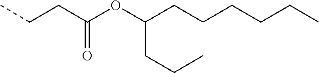 |
| Lipid 23A | 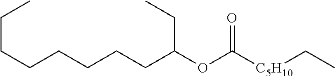 | 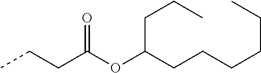 |

TABLE 4-2
R (O-acyl) and R′ (N-acyl) groups of Lipids 24, 25, 25A, 26, 27, 28, 29, and 30.

| Lipid # | R (O-acyl groups) | R′ (N-acyl group) |
|---|---|---|
| Lipid 24 | 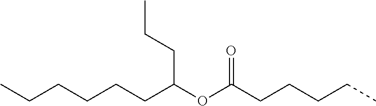 | 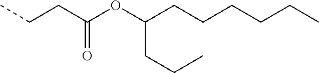 |
| Lipid 25 | 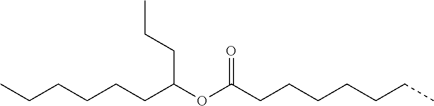 | 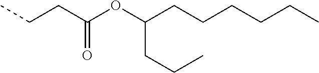 |
| Lipid 25A | 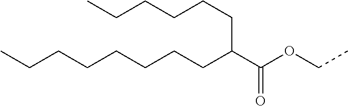 | 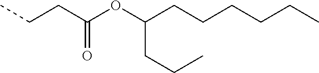 |
| Lipid 26 | 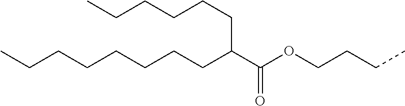 | 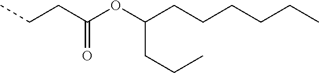 |
| Lipid 27 | 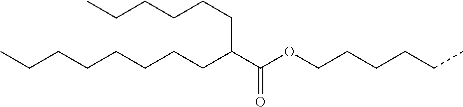 | 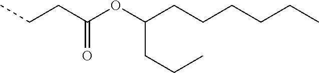 |
| Lipid 28 | 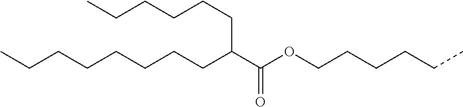 | 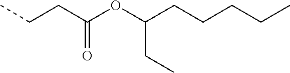 |
| Lipid 29 | 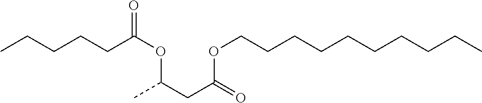 [SMILES: CCCCCCCCCCOC(=O)C[C@H](C)OC(=O)CCCCC] | 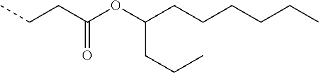 |
| Lipid 30 | 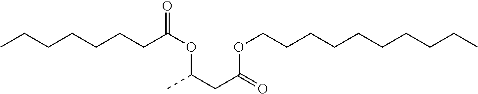 [SMILES: CCCCCCCCCCOC(=O)C[C@H](C)OC(=O)CCCCCCC] | 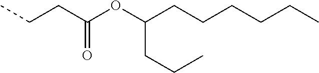 |

TABLE 4-3
R (O-acyl) and R′ (N-acyl) groups of Lipids 31 to 38, 37A, and 38A.

| Lipid # | R (O-acyl groups) | R′ (N-acyl group) |
|---|---|---|
| Lipid 31 | 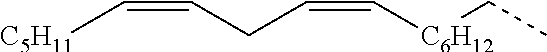 | 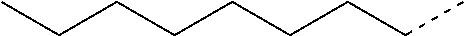 |
| Lipid 32 | 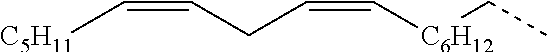 | 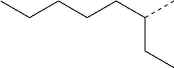 |
| Lipid 33 | 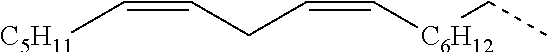 | 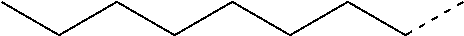 |
| Lipid 34 | 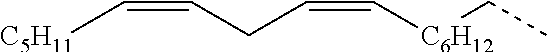 | 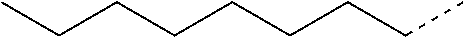 |
| Lipid 35 | 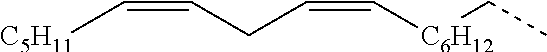 | 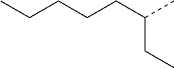 |
| Lipid 36 | 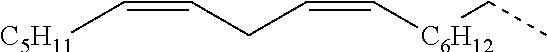 | 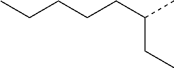 |
| Lipid 37 | 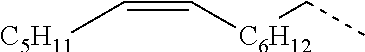 | 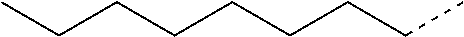 |
| Lipid 38 | 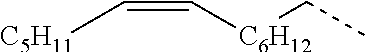 | 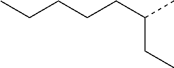 |
| Lipid 37A | 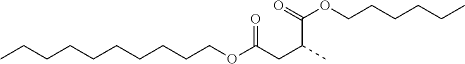 [SMILES: CCCCCCCCCCOC(=O)C[C@@H](C)C(=O)OCCCCCC] | 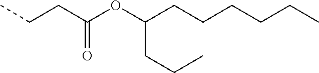 |
| Lipid 38A | 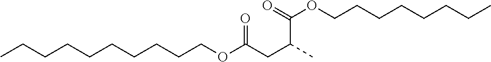 [SMILES: CCCCCCCCCCOC(=O)C[C@@H](C)C(=O)OCCCCCCCC] | 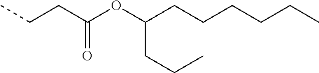 |

TABLE 5
Expected and observed mass (m/z) of named ionizable lipids.

| Entry | Compound code | Expected mass (g/mol) | Observed mass (m/z) |
|---|---|---|---|
| 1 | Lipid 1 | 854.75 | 855.7, 856.7, 857.7 |
| | | | (M + 1, M + 2, M + 3) |
| 2 | Lipid 2 | 840.73 | 841.7, 842.7, 843.7 |
| | | | (M + 1, M + 2, M + 3) |
| 3 | Lipid 3 | 840.73 | 841.7, 842.7, 843.7 |
| | | | (M + 1, M + 2, M + 3) |
| 4 | Lipid 4 | 845.39 | 845.7, 846.7, 847.7 (M, |
| | | | M + 1, M + 2) |
| 5 | Lipid 5 (S) isomer | 827.33 | 827.7, 828.7, 829.7 (M, |
| | (Lipid 5A) | | M + 1, M + 2) |
| 6 | Lipid 6 | 868.76 | 869.7, 870.7, 871.7 |
| | | | (M + 1, M + 2, M + 3) |
| 7 | Lipid 7 | 868.76 | 869.7, 870.7, 871.7 |
| | | | (M + 1, M + 2, M + 3) |
| 8 | Lipid 8 | 854.75 | 855.7, 856.7, 857.7 |
| | | | (M + 1, M + 2, M + 3) |
| 9 | Lipid 9 | 940.78 | 941.7, 942.7, 943.7 |
| | | | (M + 1, M + 2, M + 3) |
| 10 | Lipid 10 (S) isomer | 912.75 | 913.7, 914.7, 915.7 |
| | (Lipid 10A) | | (M + 1, M + 2, M + 3) |
| 11 | Lipid 11 (S) isomer | 970.76 | 971.7, 972.7, 973.7 |
| | (Lipid 11A) | | (M + 1, M + 2, M + 3) |
| 12 | Lipid 12 | 999.51 | 999.0, 1001, 1002 |
| | | | (M + 1, M + 2, M + 3) |
| 13 | Lipid 13 | 984.77 | 985.7, 986.7, 987.6 |
| | | | (M + 1, M + 2, M + 3) |
| 14 | Lipid 14A | 1013.54 | 1013.1, 1014.1, 1015.1 |
| | | | (M + 1, M + 2, M + 3) |
| 15 | Lipid 15 | 944.82 | 945.1, 946.1, 947.1 |
| | | | (M + 1, M + 2, M + 3) |
| 16 | Lipid 16 | 916.78 | 917.2, 918.2, 919.2 |
| | | | (M + 1, M + 2, M + 3) |
| 17 | Lipid 17A | 896.67 | 897.9, 898.9, 899.9 |
| | | | (M + 1, M + 2, M + 3) |
| 18 | Lipid 18A | 952.73 | 953.7, 954.7, 955.7 |
| | | | (M + 1, M + 2, M + 3) |
| 19 | Lipid 19A | 1008.80 | 1009.8, 1010.8, 1011.8 |
| | | | (M + 1, M + 2, M + 3) |
| 20 | Lipid 20A | 1064.9 | 1065.7, 1066.7, 1067.7 |
| | | | (M + 1, M + 2, M + 3) |
| 21 | Lipid 21A | 1008.80 | 1009.7, 1010.7, 1011.7 |
| | | | (M + 1, M + 2, M + 3) |
| 22 | Lipid 22 | 980.76 | 981.7, 982.7, 983.7 |
| | | | (M + 1, M + 2, M + 3) |
| 23 | Lipid 23A | 1036.8 | 1037.7, 1038.6, 1039.7 |
| | | | (M + 1, M + 2, M + 3) |
| 24 | Lipid 19 (Lipid 24A) | 892.78 | 893.7, 894.7, 895.7 |
| | | | (M + 1, M + 2, M + 3) |
| 25 | Lipid 25A | 1008.80 | 1009.7, 1010.7, 1011.7 |
| | | | (M + 1, M + 2, M + 3) |
| 26 | Lipid 20 (Lipid 26) | 1064.86 | 1065.1, 1066.1, |
| | | | 1067.1, 1068.1 (M + 1, |
| | | | M + 2, M + 3, M + 4) |
| 27 | Lipid 27 | 1120.92 | 1121.9, 1122.9, 1123.9 |
| | | | (M + 1, M + 2, M + 3) |
| 28 | Lipid 28 | 1092.9 | 1093.8, 1094.8, 1095.8 |
| | | | (M + 1, M + 2, M + 3) |
| 29 | Lipid 29 | 1124.81 | 1125.8, 1126.8, 1127.8 |
| | | | (M + 1, M + 2, M + 3) |
| 30 | Lipid 30 | 1180.87 | NA |
| 31 | Lipid 31 | 854.75 | 855.1, 856.1, 857.1 |
| | | | (M + 1, M + 2, M + 3) |
| 32 | Lipid 32 | 854.75 | 855.7, 856.7, 857.7 |
| | | | (M + 1, M + 2, M + 3) |
| 33 | Lipid 33 | 840.73 | 841.7, 842.7, 843.7 |
| | | | (M + 1, M + 2, M + 3) |
| 34 | Lipid 34 | 868.76 | 869.7, 870.7, 871.7 |
| | | | (M + 1, M + 2, M + 3) |
| 35 | Lipid 35 | 914.77 | NA |
| 36 | Lipid 36 | 884.76 | NA |
| 37 | Lipid 37A | 1153.63 | 1153.8, 1154.8, 1155.8 |
| | | | (M + 1, M + 2, M + 3) |
| 38 | Lipid 38A | 1209.74 | NA |

### Synthesis of Lipids 1-16 by N-Acylation of Intermediates 13-0 or 13-11b

N-acylation of intermediates 13-0 and 13-11b with compounds R′CO₂H or R′COCl (R′ structures shown in Table 2 and Table 3) yielded lipids 1 through 16 as described in examples below.

### Synthesis of Lipids 1, 3, 4, 5, 6, and 7 by N-Acylation of Intermediate 13-0 Using the Corresponding Acid Chlorides

### Synthesis of Lipid 1

Lipid 1 was synthesized as provided in scheme 6 below and as follows. Starting material, 131-1 (0.75 mmol, 130 mg, 1.0 eq) was converted to the acid chloride (Step 1) using oxalyl chloride (3.7 mmol, 320 μl, 5 eq,) and DMF (10 μl, catalytic) in 6 mL of benzene. Product (143 mg, 98%) showed only one spot on TLC (as methyl ester) and was used without further purification for acylation (step 2) of intermediate 13-0. Intermediate 13-0 (0.35 mmol, 250 mg, 1.0 eq.) was acylated with crude acid chloride, 131-1 (0.75 mmol, 143 mg, 1.7 eq.) using TEA (240 μL, 5 eq, 1.8 mmol) and DMAP (10 mg, catalytic amount). Crude product was purified by column chromatography (2×) yielding 124 mg (76%) of pure Lipid 1 (>99% purity by LC-ELSD) and characterized by proton NMR and Mass Spectrometry (see FIG. 4A-1 for Lipid 1 NMR spectrum, and Table 5 for product mass).

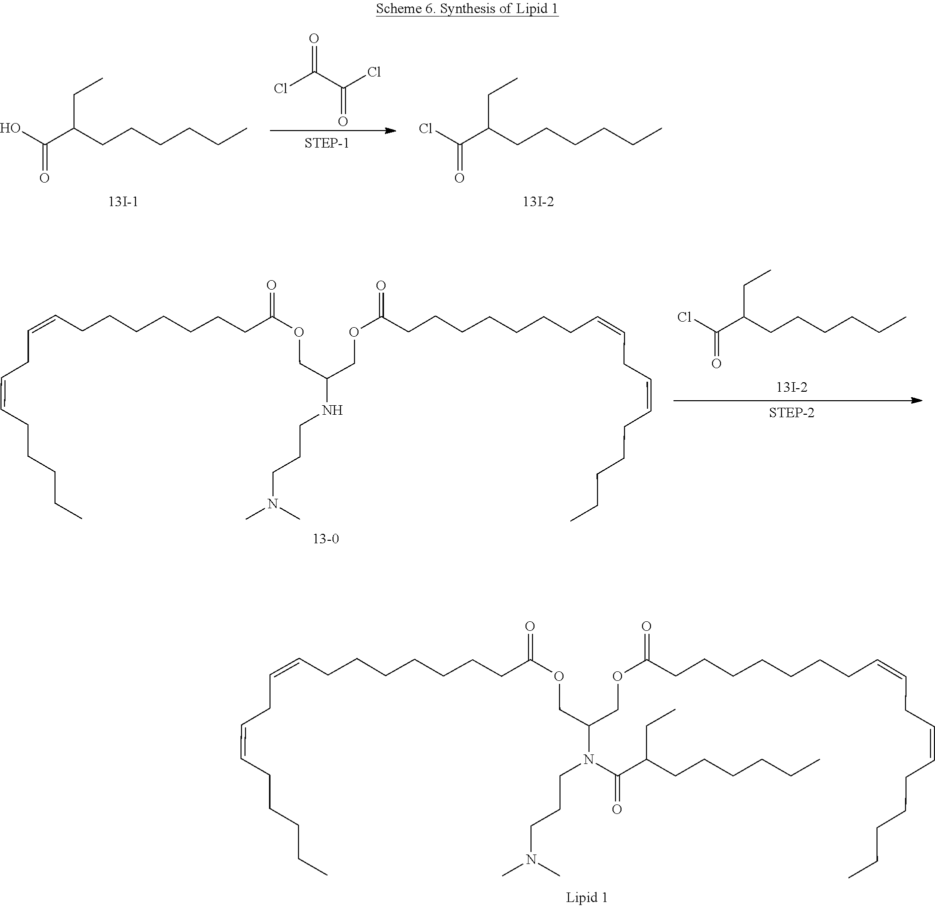 [SMILES: CCCCCCC(CC)C(=O)O ; O=C(Cl)C(=O)Cl ; CCCCCCC(CC)C(=O)Cl ; CCCCC/C=C\C/C=C\CCCCCCCC(=O)OCC(COC(=O)CCCCCCC/C=C\C/C=C\CCCCC)NCCCN(C)C ; CCCCCCC(CC)C(=O)Cl ; CCCCC/C=C\C/C=C\CCCCCCCC(=O)OCC(COC(=O)CCCCCCC/C=C\C/C=C\CCCCC)N(CCCN(C)C)C(=O)C(CC)CCCCCC]

### Synthesis of Lipid 3

Lipid 3 was synthesized as provided in scheme 7 below and as follows. Starting material, 13-13 (8.3 mmol, 1.30 g, 1.0 eq) was converted to the acid chloride, 13-13a (Step 1) using oxalyl chloride (2.8 mmol, 2.4 ml, 5 eq.) and DMF (100 μl, catalytic) in 60 mL of benzene. Product (1.44 g, 98%) showed only one spot on TLC (as methyl ester) and was used without further purification for acylation (step 2) of intermediate 13-0. Intermediate 13-0 (5.4 mmol, 3.78 g, 1.0 eq.) was acylated with crude acid chloride, 13-13a (1.44 g, 1.5 eq, 8.1 mmol) using TEA (3.76 mL, 5 eq, 27 mmol) and DMAP (50 mg, cat, catalytic amount) in benzene (100 mL). Crude product was purified by column chromatography (2×) yielding 2.1 g (46.3%) of pure Lipid 3 (≥99% purity by LC-ELSD) and characterized by proton NMR and Mass Spectrometry (see FIG. 4B-1 for Lipid 3 NMR spectrum, FIG. 4B-2 for Lipid 3 LC-MS, and Table 5 for product mass).

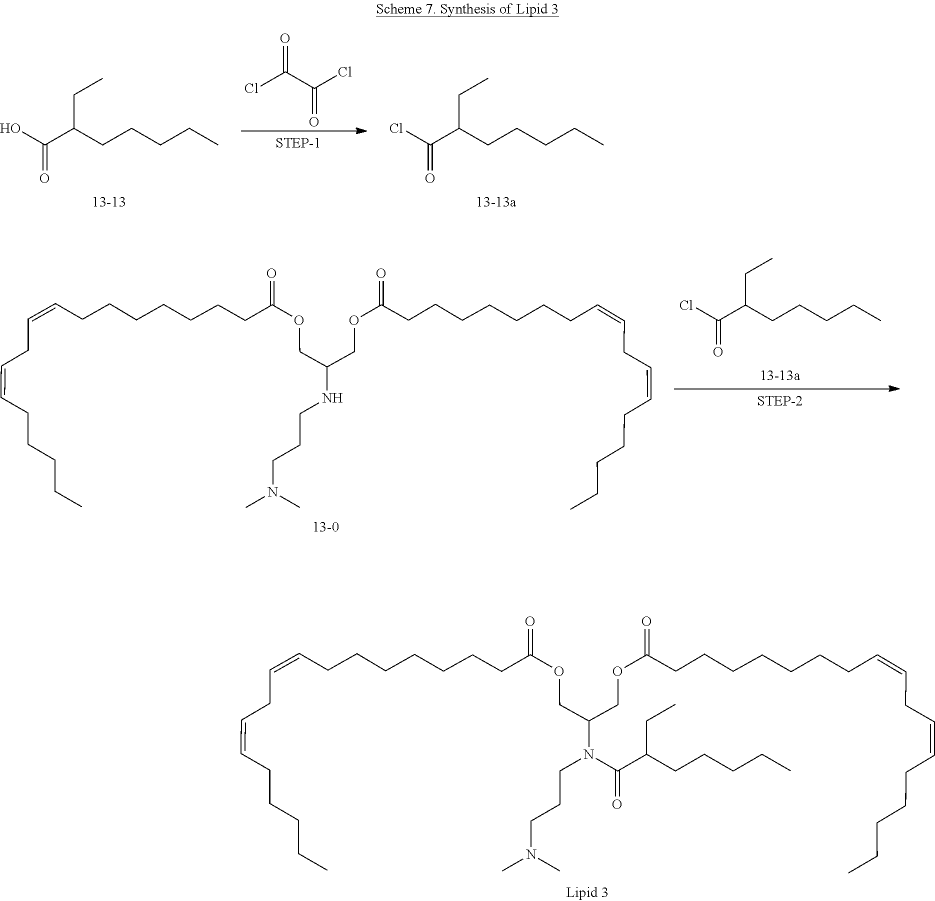 [SMILES: CCCCCC(CC)C(=O)O ; O=C(Cl)C(=O)Cl ; CCCCCC(CC)C(=O)Cl ; CCCCC/C=C\C/C=C\CCCCCCCC(=O)OCC(COC(=O)CCCCCCC/C=C\C/C=C\CCCCC)NCCCN(C)C ; CCCCCC(CC)C(=O)Cl ; CCCCC/C=C\C/C=C\CCCCCCCC(=O)OCC(COC(=O)CCCCCCC/C=C\C/C=C\CCCCC)N(CCCN(C)C)C(=O)C(CC)CCCCC]

### Synthesis of Lipid 4

Lipid 4 was synthesized as provided in scheme 7 below and as follows. Starting material, 13-18 (0.95 mmol, 150 mg, 1 eq.) was converted to the acid chloride, 13-18′ (Step 1) using oxalyl chloride (3.23 mmol, 227 μl, 3.4 eq.) and DMF (10 μl, catalytic) in 6 mL of benzene. Product showed only one spot on TLC (as methyl ester) and was used without further purification for acylation (step 2) of intermediate 13-11b. Intermediate 13-11b (0.63 mmol, 444 mg, 1.0 eq.) was acylated with crude acid chloride, 13-18′ (167 mg, 1.5 eq, 0.95 mmol) using TEA (445 μL, 5.0 eq, 3.2 mmol) and DMAP (10 mg, catalytic amount) in benzene (10 mL). Crude product was purified by column chromatography (5×) yielding 140 mg (26%) of pure Lipid 4 (97% purity by LC-ELSD) and characterized by proton NMR and Mass Spectrometry (see FIG. 4C-1 for Lipid 4 NMR spectrum, FIG. 4C-2 for Lipid 4 LC-MS, and Table 5 for product mass).

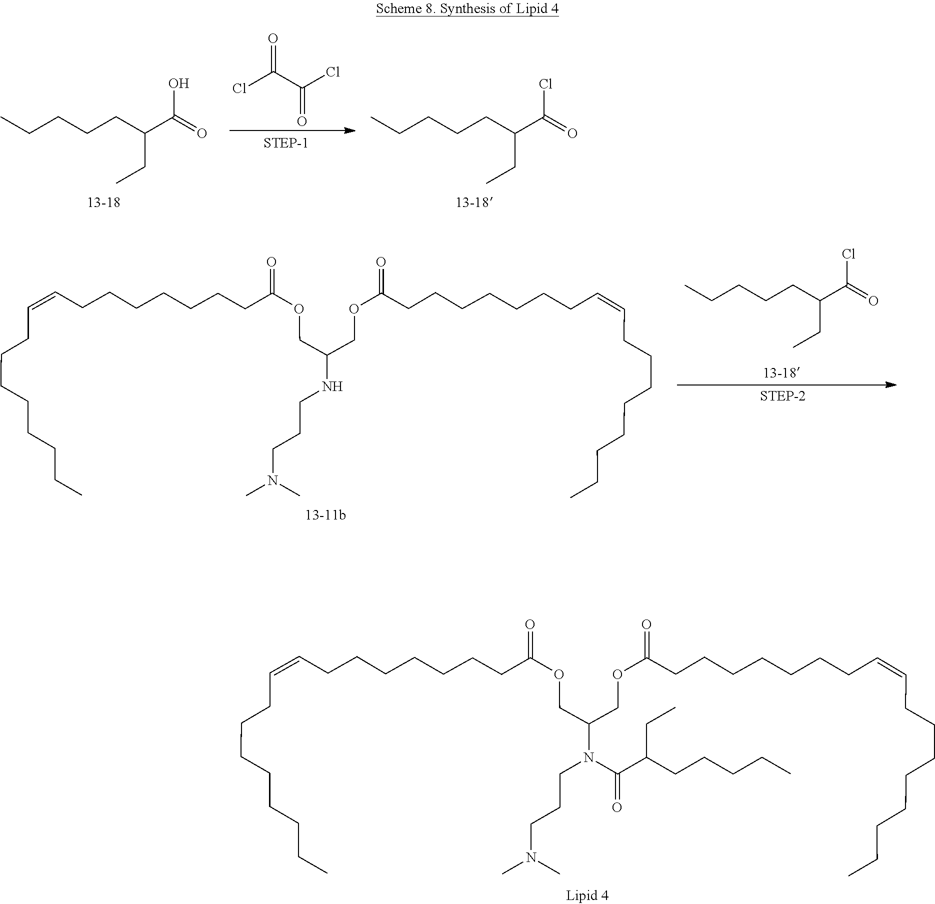 [SMILES: CCCCCC(CC)C(=O)O ; O=C(Cl)C(=O)Cl ; CCCCCC(CC)C(=O)Cl ; CCCCCCCC/C=C\CCCCCCCC(=O)OCC(COC(=O)CCCCCCC/C=C\CCCCCCCC)NCCCN(C)C ; CCCCCC(CC)C(=O)Cl ; CCCCCCCC/C=C\CCCCCCCC(=O)OCC(COC(=O)CCCCCCC/C=C\CCCCCCCC)N(CCCN(C)C)C(=O)C(CC)CCCCC]

### Synthesis of Lipid 5 and its (S) Isomer

The(S) isomer of lipid 5 was synthesized as provided in scheme 9-1 below and as follows. Starting material, Ethyl hexenoic acid 13m-1 (110 mg, 1.0 eq, 0.75 mmol) was converted to the acid chloride, 13m-2 (Step 1) using oxalyl chloride (320 μL, 1.0 eq, 3.7 mmol) and DMF (20 μl, catalytic) under reflux for 2 hours in 3 mL of benzene. Product showed only one spot on TLC (as methyl ester) and was used without further purification for acylation (step 2) of intermediate 13-0. Intermediate 13-0 (250 mg, 1.0 eq, 0.35 mmol) was acylated with crude acid chloride, 13m-2 (120 mg, 1.8 eq, 0.75 mmol) using TEA (240 μL, 5.0 eq, 1.8 mmol) and DMAP (10 mg, catalytic amount) in 10 mL benzene, overnight at room temperature. Crude product was purified by column chromatography (2×) yielding 95 mg (32%) of pure Lipid 5 (≥99% purity by LC-ELSD) and characterized by proton NMR and Mass Spectrometry (see FIG. 4D-1 for Lipid 5 NMR spectrum, FIG. 4D-2 for Lipid 5 LC-MS, and Table 5 for product mass).

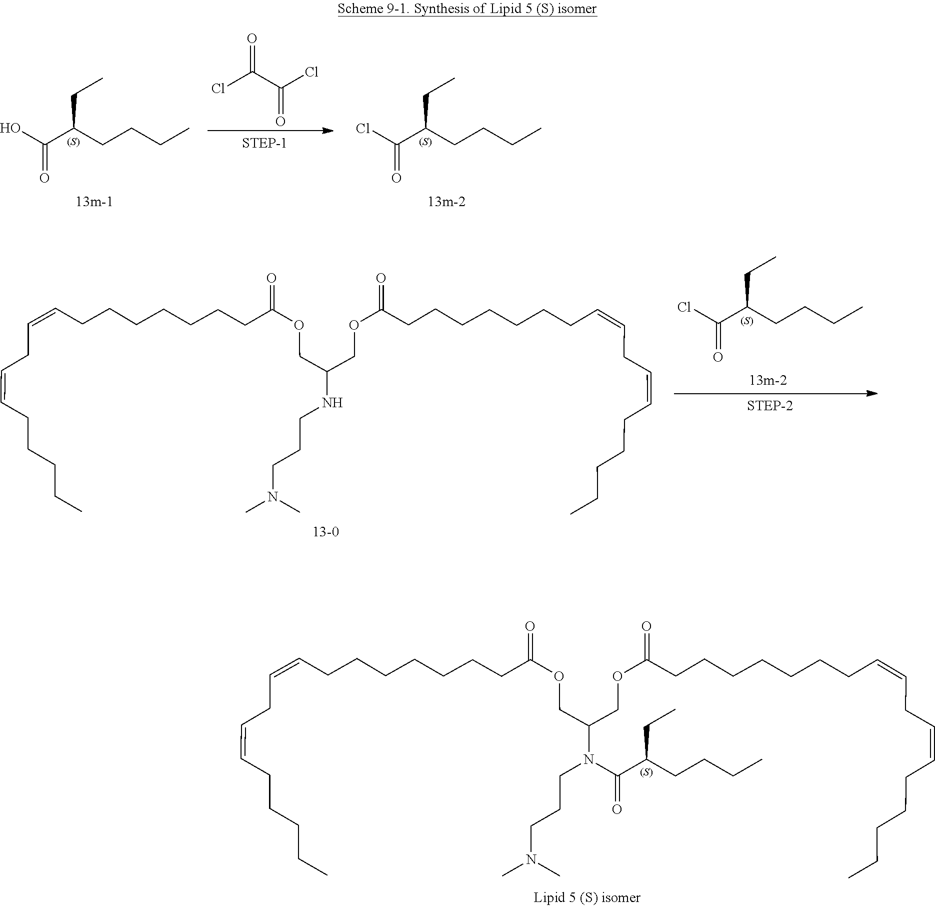

Lipid 5 as a racemic mixture was synthesized similarly as provided in scheme 9-2 below.

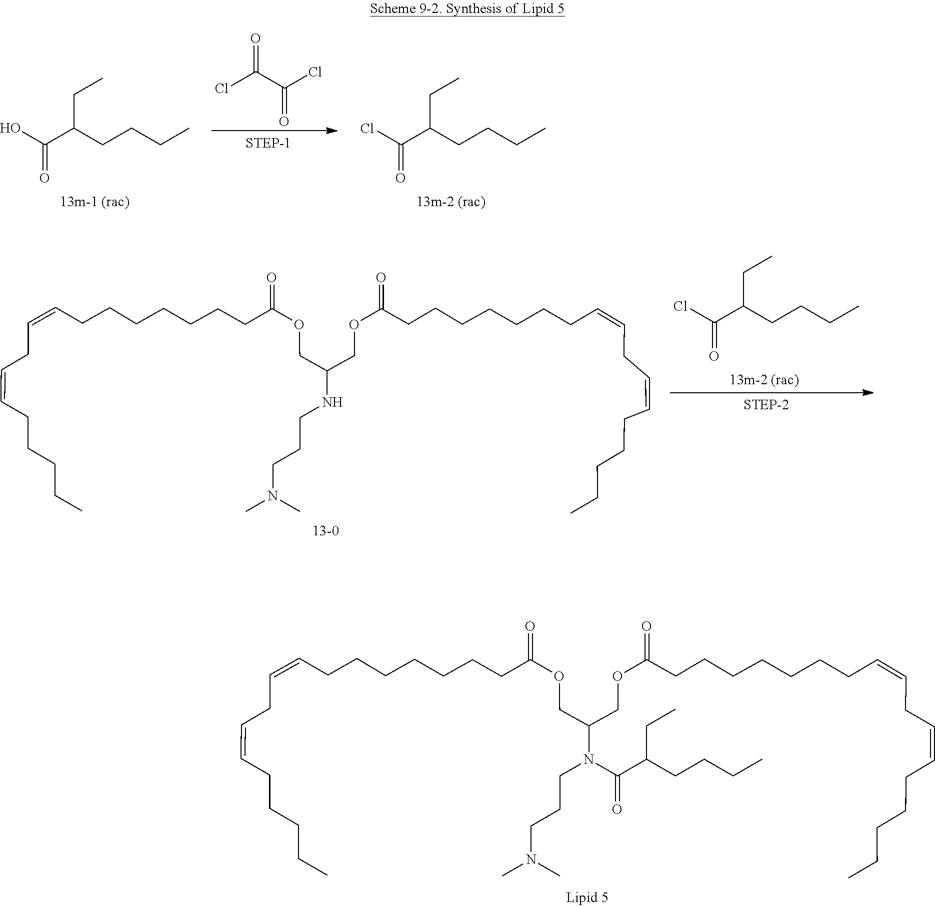

### Synthesis of Lipid 6

Lipid 6 was synthesized as provided in scheme 10 below and as follows. Starting material, 2-ethylnonanoic acid 13-14 (132 mg, 0.17 mmol, 1 eq.) was converted to the acid chloride, 13-14′ (Step 1) using oxalyl chloride (207 μl, 3.4 eq, 2.4 mmol) and DMF (10 μl, catalytic quantity) in 6 mL of benzene. Product showed only one spot on TLC (as methyl ester) and was used without further purification for acylation (step 2) of intermediate 13-0. Intermediate 13-0 (0.47 mmol, 330 mg, 1 eq.) was acylated with crude acid chloride, 13-14′ (145 mg, 1.5 eq, 0.7 mmol) using TEA (327 μL, 5.0 eq, 2.4 mmol) and DMAP (10 mg, catalytic amount) in 10 mL benzene. Crude product was purified by column chromatography (2×) yielding 75 mg (18%) of pure Lipid 6 (≥99% purity by LC-ELSD) and characterized by proton NMR and Mass Spectrometry (see FIG. 4E-1 for Lipid 6 NMR spectrum, FIG. 4E-2 for Lipid 6 LC-MS, and Table 5 for product mass).

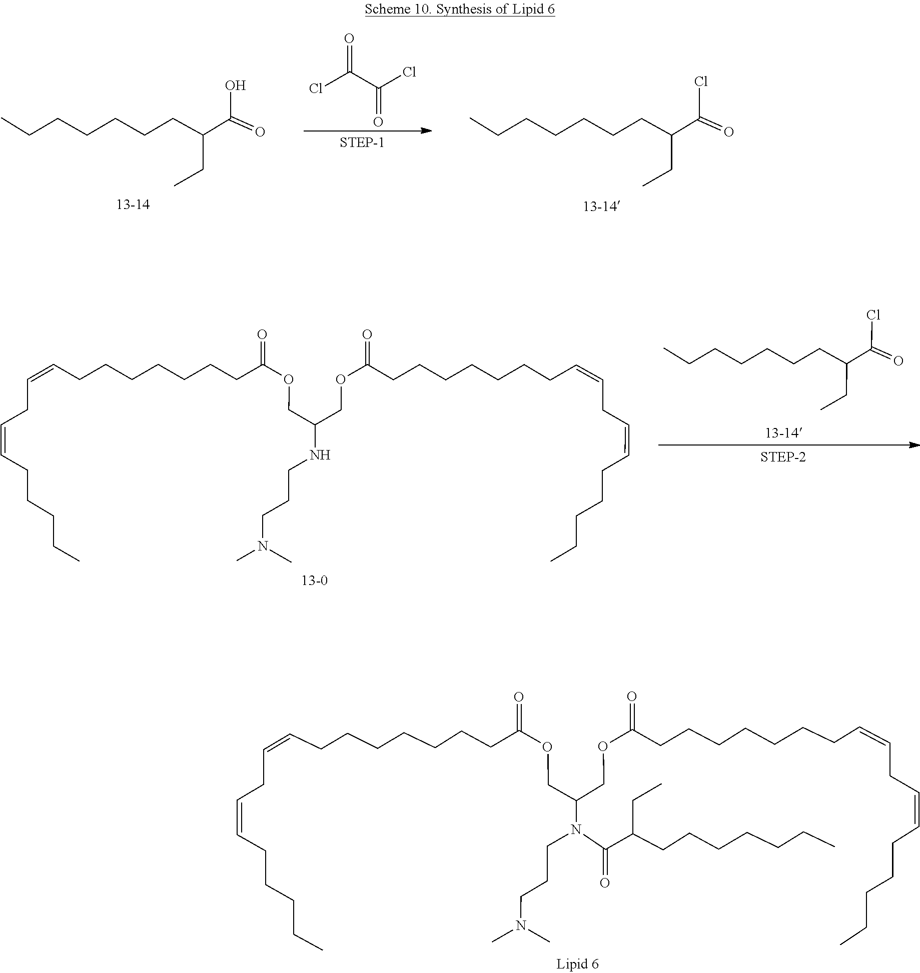 [SMILES: CCCCCCCC(CC)C(=O)O ; O=C(Cl)C(=O)Cl ; CCCCCCCC(CC)C(=O)Cl ; CCCCC/C=C\C/C=C\CCCCCCCC(=O)OCC(COC(=O)CCCCCCC/C=C\C/C=C\CCCCC)NCCCN(C)C ; CCCCCCCC(CC)C(=O)Cl ; CCCCC/C=C\C/C=C\CCCCCCCC(=O)OCC(COC(=O)CCCCCCC/C=C\C/C=C\CCCCC)N(CCCN(C)C)C(=O)C(CC)CCCCCCC]

### Synthesis of Lipid 7

Lipid 7 was synthesized as provided in scheme 11 below and as follows. Starting material, heptanoic acid, 13-15 (23.1 mmol, 3.0 g, 1 eq.) was alkylated (step 1) with n-butyl bromide, 13-16 ((2.5 mL, 1.0 eq, 23.1 mmol) and 2.5 M n-butyl lithium in hexane (20.0 mL, 2.2 eq, 51 mmol) using diisopropylamine (7.2 mL, 2.2 eq, 51 mmol) in HMPA (4.4 mL) and 30 mL THF. 1.5 g (35%) of 2-butyl heptanoic acid, 13-17, was isolated from reaction mixture by flash chromatography. Intermediate 13-17 (360 mg, 0.94 mmol, 1 eq.) was converted to the acid chloride, 13-17′ (Step 2) using oxalyl chloride (6.6 mmol, 568 μl, 3.4 eq.) and DMF (5 μl, catalytic) in 3 mL of benzene. Product showed only one spot on TLC (as methyl ester) and was used without further purification for acylation (step 3) of intermediate 13-0. Intermediate 13-0 (0.64 mmol, 450 mg, 1 eq.) was acylated with crude acid chloride, 13-17′ (395 mg, 3.0 eq, 1.94 mmol), TEA (446 μL, 5.0 eq, 3.2 mmol), DMAP (10 mg) in 10 mL of benzene. Crude product was purified by column chromatography (2×) yielding 228 mg (41%) of pure Lipid 7 (≥99% purity by LC-ELSD) and characterized by proton NMR and Mass Spectrometry (see FIG. 4F-1 for Lipid 7 NMR spectrum, FIG. 4F-2 for Lipid 7 LC-MS, and Table 5 for product mass).

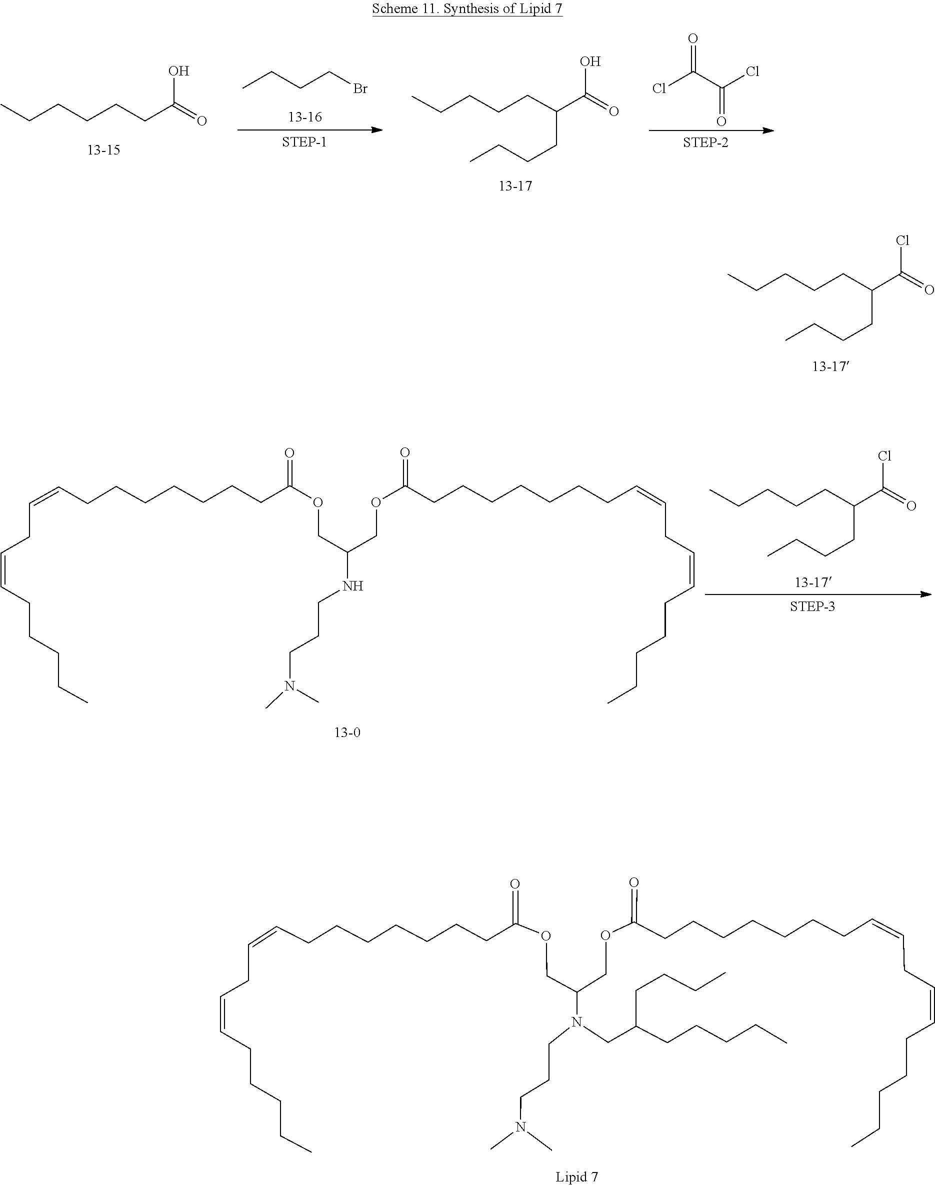

### Synthesis of Lipids 2, 8, 9 and 10 by N-Acylation of Intermediate 13-0 Using Carbodiimide Activation of the Corresponding Carboxylic Acids

### Synthesis of Lipid 2

Lipid 2 was synthesized as provided in scheme 12 below and as follows. Intermediate 13-0 (0.14 mmol, 320 mg, 1.0 eq.) was acylated with nonanoic acid 13-12 (1.15 mmol, 198 μL, 2.5 eq.), EDCI (1.15 mmol, 221 mg, 2.5 eq.), DIPEA (1.15 mmol, 198 μL, 2.5 eq.), and DMAP (0.05 mmol, 6.4 mg, 0.1 eq.) in 5 mL DCM. Crude product was purified by column chromatography (3×) yielding 107 mg (%) of pure Lipid 2 (≥99% purity by LC-ELSD) and characterized by proton NMR and Mass Spectrometry (see FIG. 4G-1 for Lipid 2 NMR spectrum, FIG. 4G-2 for Lipid 2 LC-MS, and Table 5 for product mass).

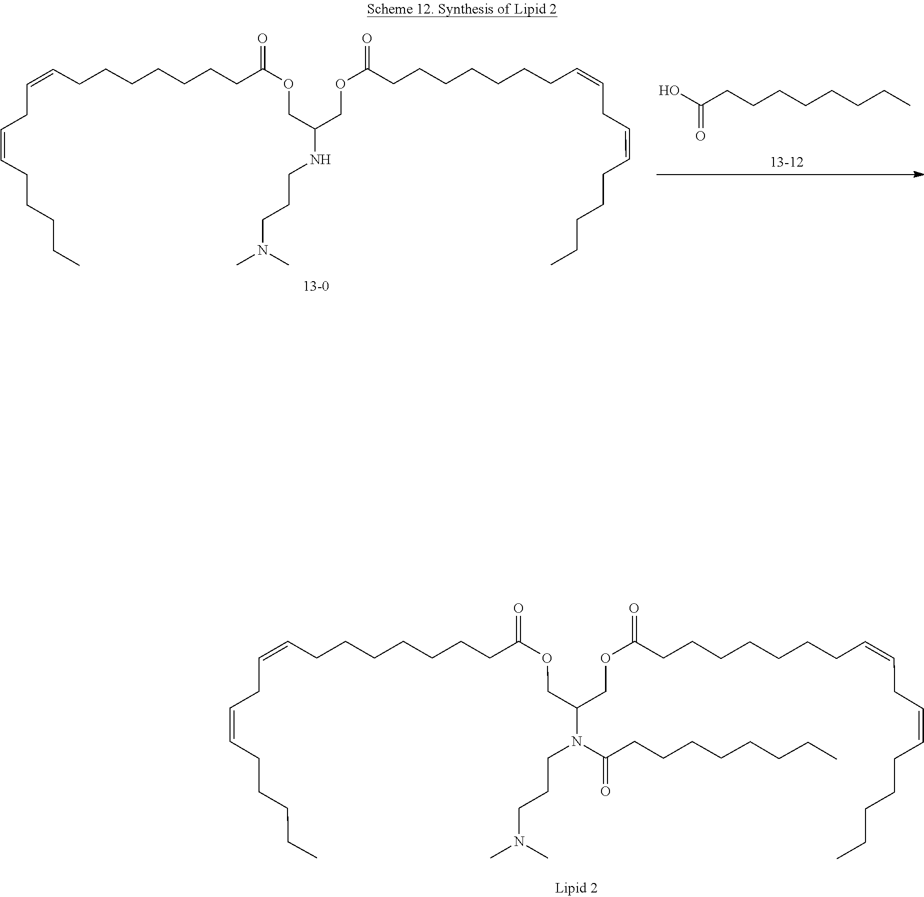 [SMILES: CCCCC/C=C\C/C=C\CCCCCCCC(=O)OCC(COC(=O)CCCCCCC/C=C\C/C=C\CCCCC)NCCCN(C)C ; CCCCCCCCC(=O)O ; CCCCC/C=C\C/C=C\CCCCCCCC(=O)OCC(COC(=O)CCCCCCC/C=C\C/C=C\CCCCC)N(CCCN(C)C)C(=O)CCCCCCCC]

### Synthesis of Lipid 8

Lipid 8 was synthesized as provided in scheme 13 below and as follows. Alkene, 13-48 was accessed via the HWE reaction (step 1) of octan-3-one, 13-46 (2 g, 15.6 mmol) with ethyl 2-(diethoxyphosphoryl) acetate, 13-47 (7.0 g, 2.0 eq, 31.2 mmol), 2M NaHMDS in THF (15.6 mL, 2.0 eq, 31.2 mmol), and 9 ml THF solvent. Reaction workup yielded 2.38 g (77%) of 13-48 confirmed by NMR, product mass and single TLC spot. Alkene, 13-48 (5.1 mmol, 1 g, 1 eq.) was hydrogenated (step 2) using Pd/C (50 mg) in 8 mL ethyl acetate yielding intermediate 13-48 (958 mg, 77%). Ester hydrolysis (step 3) of 13-49 (5.1 mmol, 412 mg) using THF/MeOH/1M LiOH (3.0/2.0/3.0 mL) yielded carboxylic acid intermediate 13-50 (336 mg, 95%). Intermediate 13-0 (0.33 mmol, 234 mg) was acylated with 13-50 (0.66 mmol, 115 mg, 2.0 eq.) using EDCI (0.66 mmol, 102 mg, 2.0 eq.), DIPEA (0.66 mmol, 114 μL, 2.0 eq.), DMAP (0.33 mmol, 41 mg, 1.0 eq.), in 2 mL DCM yielding 77 mg (27%) of pure Lipid 8 (≥99% purity by LC-ELSD) and characterized by proton NMR and Mass Spectrometry (see FIG. 4H-1 for Lipid 8 NMR spectrum, FIG. 4H-2 for Lipid 8 LC-MS, and Table 5 for product mass).

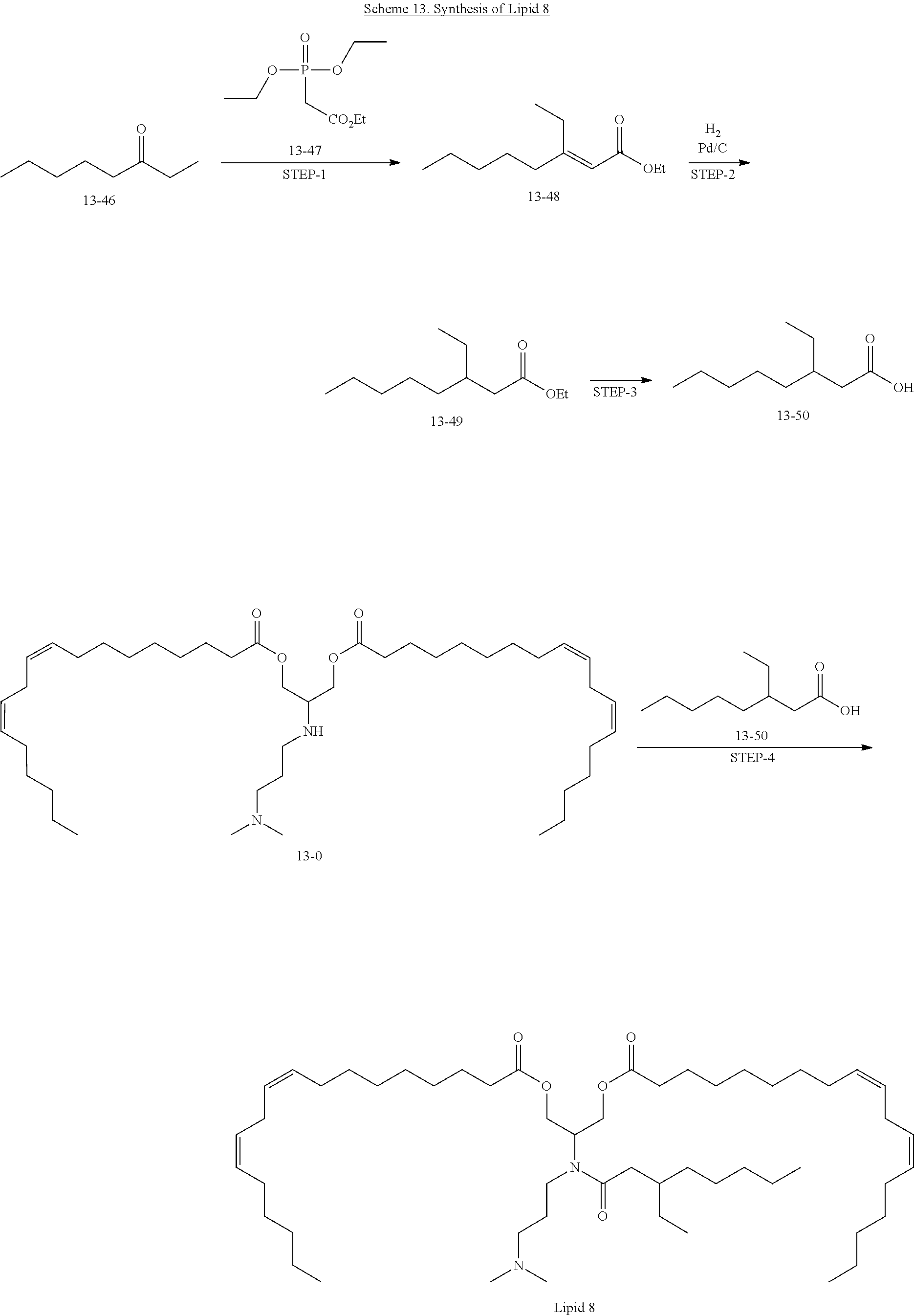

### Synthesis of Lipid 9

Lipid 9 was synthesized as provided in scheme 14 below and as follows. Starting material, decan-4-ol, 13-29 (32.0 mmol, 5.0 g, 1.0 eq.) was acylated with succinic acid, 13-30 (6.3 g, 2.0 eq, 63.0) using DMAP (3.55 g, 1.0 eq, 32.0 mmol) and pyridine (5.0 ml) in 5 mL THF. Crude product was purified by column chromatography (1×) to obtain 4.26 g (81%) of pure acid intermediate 13-31. Intermediate 13-0 (2.1 mmol, 1.5 g, 1 eq.) was acylated with 13-31 (2.13 mmol, 0.554 g, 1.1 eq), using DIPEA (745 μL, 4.26 mmol, 2.5 eq), EDCI (820 mg, 4.26 mmol, 2.5 eq), and DMAP (480 mg, 0.43 mmol, 0.25 eq), in 50 mL DCM. Crude product was purified by column chromatography (3×) yielding 1.4 g (73%) of pure Lipid 9 (≥99% purity by LC-ELSD) and characterized by proton NMR and Mass Spectrometry (see FIG. 4I-1 for Lipid 9 NMR spectrum, FIG. 41-2 for Lipid 9 LC-MS, and Table 5 for product mass).

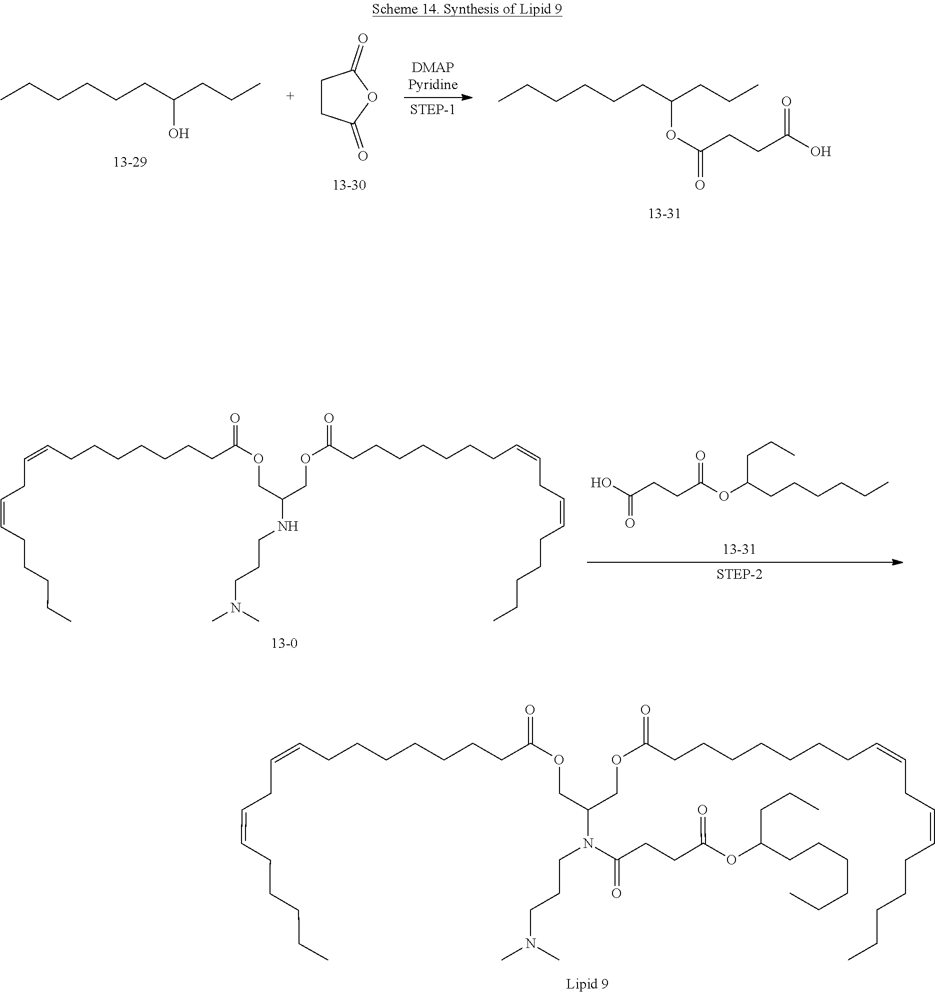

### Synthesis of Lipid 10 and its (s) Isomer

The (S) isomer of lipid 10 was synthesized as provided in scheme 15-1 below and as follows. Starting material, Octan-3-ol, 13-46 (2.0 g, 1.0 eq, 15.3 mmol) was acylated with succinic acid, 13-30 (3.1 g, 2.0 eq, 30.6 mmol) using DMAP (1.72 g, 1.0 eq, 15.3 mmol) and pyridine (2.0 ml) in 2 mL THF and 6 mL DCM. Crude product was purified by column chromatography (1×) to obtain 1.1 g (31%) of pure acid intermediate 13-47. Intermediate 13-0 (250 mg, 1.0 eq, 0.36 mmol) was acylated with 13-47 (123 mg, 1.5 eq, 0.53 mmol), using EDCI (207 mg, 3.0 eq, 1.80 mmol), DIPEA (188 μL, 3.0 eq, 1.8 mmol) and DMAP (15.0 mg, 3.0 eq, 0.018 mmol), in 5 mL DCM. Crude product was purified by column chromatography (2×) yielding 261 mg (54%) of pure Lipid 10 (≥99% purity by LC-ELSD) and characterized by proton NMR and Mass Spectrometry (see FIG. 4J-1 for Lipid 10 NMR spectrum, FIG. 4J-2 for Lipid 10 LC-MS, and Table 5 for product mass).

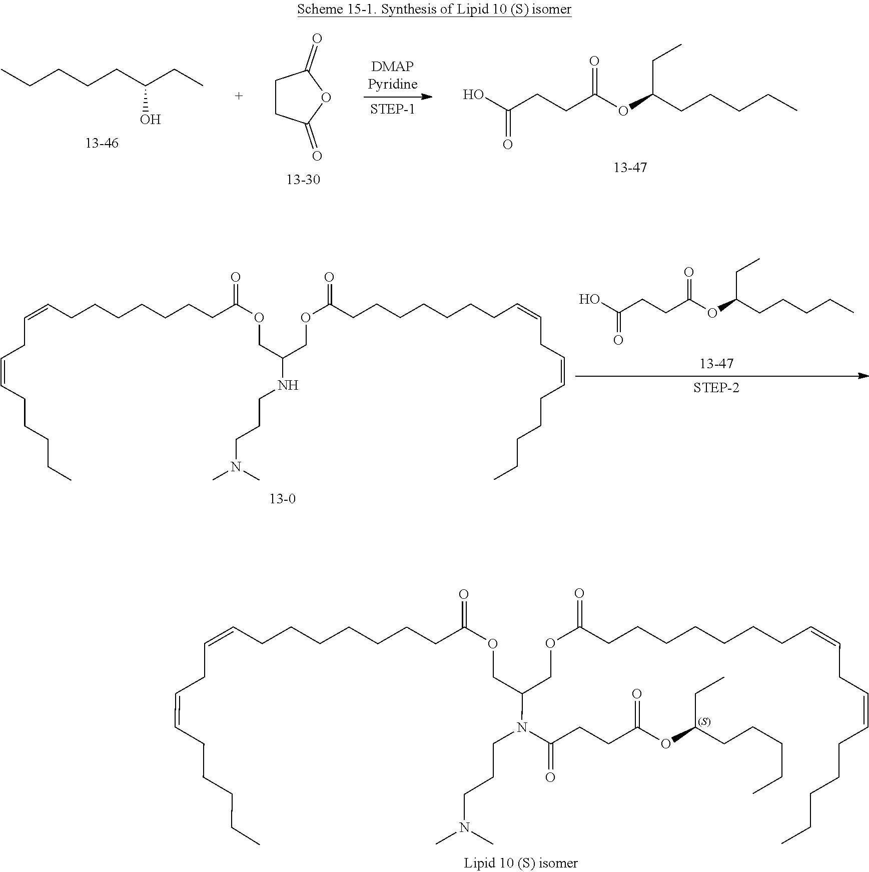

Lipid 10 as a racemic mixture was synthesized similarly as provided in scheme 15-2 below. Starting material, octan-3-ol, 13-46 (2.0 g, 1.0 eq, 15.3 mmol) was acylated with succinic acid, 13-30 (3.1 g, 2.0 eq, 30.6 mmol) using DMAP (1.72 g, 1.0 eq, 15.3 mmol) and pyridine (2.0 ml) in 2 mL THF and 6 mL DCM to obtain intermediate 13-47. Crude product was purified by column chromatography (1×) to obtain 1.1 g (31%) of pure acid intermediate 13-47. 13-0 (250 mg, 1.0 eq, 0.36 mmol) was acylated with 13-38 (123 mg, 1.5 eq, 0.53 mmol) using DIPEA (188 μL, 3.0 eq, 1.8 mmol), EDCI (207 mg, 3.0 eq, 1.80 mmol), and DMAP (15.0 mg, 3.0 eq, 0.018 mmol), in 5 mL DCM. Crude product was purified by column chromatography (2×) yielding 261 mg (54%) of pure Lipid 10 (≥99% purity by LC-ELSD) and characterized by proton NMR and Mass Spectrometry (see FIG. 4J-1 for Lipid 10 NMR spectrum, FIG. 4J-2 for Lipid 10 LC-MS, and Table 5 for product mass).

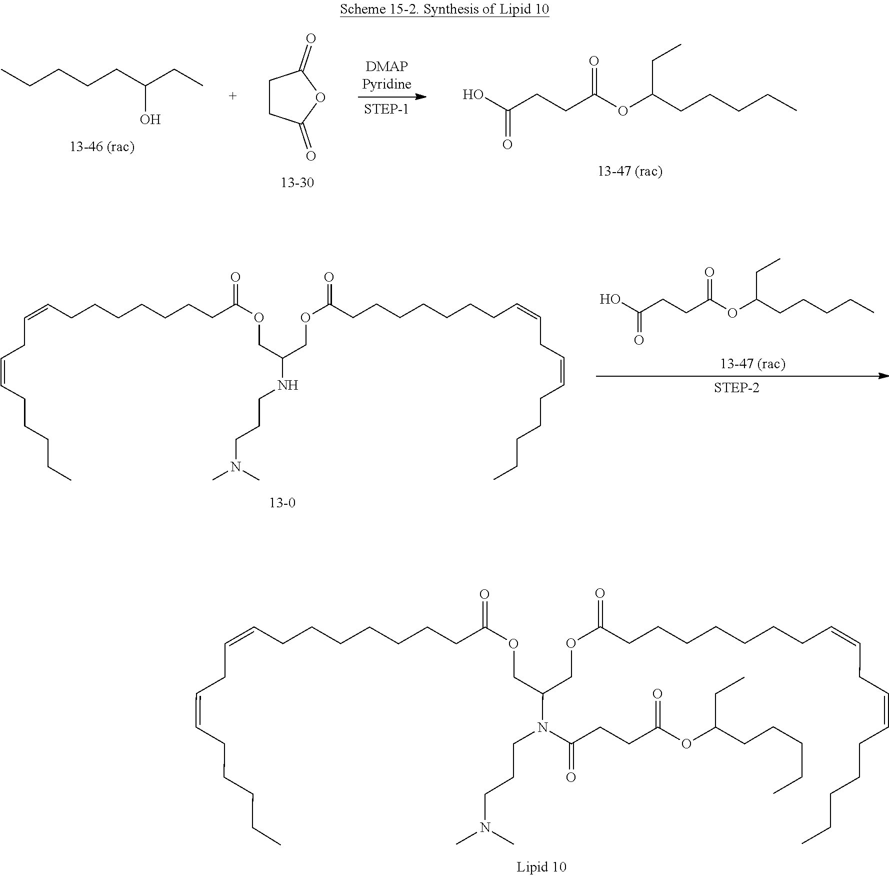

### Synthesis of Lipid 11 by N-Acylation of Intermediate 13-0 Using the Corresponding Acid Chloride

### Synthesis of Lipid 11 and its(s) Isomer

The (S) isomer of lipid 5 was synthesized as provided in scheme 16-1 below and as follows. Starting material, benzyl alcohol, 13-39′ (18.5 mmol, 2 g) was used to acylate compound 13-39 (4.8 g, 1.5 eq, 27.8 mmol) using EDCI (5.4 g, 1.5 eq, 27.8 mmol), DIPEA (4.6 mL, 1.5 eq, 27.8 mmol), and DMAP (463 mg, 0.2 eq, 3.7 mmol) yielding 3.6 g (74%) of column purified intermediate 13-40 (product confirmed by mass spectrometry and proton NMR). Intermediate, 13-40 (684 mg, 2.6 mmol, 1 eq.) was deprotected in acetic acid to obtain intermediate, 13-41 (~600 mg, quantitative and product structure was confirmed by mass spectrometry and proton NMR). Additional quantity of intermediate 13-41 was generated and 1.68 g, 7.5 mmol of 13-41 was selectively protected at the hydroxyl group using TBSCl (1.7 g, 11.25 mmol, 1.5 eq), TEA (5.3 mL, 5.0 eq, 37.5 mmol), and DMAP (92 mg, 0.75 mmol, 0.1 eq), in 20 mL DCM yielding protected intermediate 13-41a (~2.5 g, quantitative) (product mass was confirmed by mass spectrometry and proton NMR). Intermediate 13-41a (1.61 g, 4.76 mmol) was esterified with n-hexyl alcohol 13-34 (2.94 mL, 23.8 mmol, 5.0 eq) using EDCI (2.76 g, 14.2 mmol, 3.0 eq), DIPEA (1.6 mL, 2.0 eq, 9.52 mmol), and DMAP (580 mg, 4.76 mmol, 1.0 eq) in 11.0 mL of DCM to obtain 13-41b (0.95 g, 48%). Additional quantity of 13-41b was generated and total of 1.36 g (3.2 mmol) was deprotected using HF-pyridine (5.8 mL, 80.6 mmol, 25 eq.) in 30 mL THF to obtain intermediate 13-41c (837 mg, 84%). Intermediate 13-41c (456 mg, 1.48 mmol) was acylated with n-butanoyl chloride, 13-42 (760 μL, 7.4 mmol, 5.0 eq) in 4.0 mL pyridine (4.0 mL) yielding compound 13-44 (505 mg, 90%). Intermediate 13-44 (505 mg, 1.34 mmol) was deprotected using Pd/C (30 mg) in 3.0 mL ethyl acetate yielding compound 13-45 (370 mg, 96%). Compound 13-45 (188 mg, 0.65 mmol) was converted to the acid chloride intermediate using oxalyl chloride (190 μg, 3.4 eq, 2.2 mmol) and DMF (10 μL, catalytic quantity), in 3 mL Benzene. Product showed only one spot on TLC (as methyl ester) and was used without further purification for acylation (step 9) of intermediate 13-0. Intermediate 13-0 (152 mg, 0.22 mmol, 1 eq.) was acylated with crude acid chloride, 13-45′ (200 mg, 3.0 eq, 0.65 mmol), TEA (152 μL, 5.0 eq, 1.1 mmol), DMAP (10 mg) in 5 mL of benzene to obtain Lipid 11. Crude product was purified by column chromatography to yield 77 mg (37%) of pure Lipid 11 (≥99% purity by LC-ELSD) and characterized by proton NMR and Mass Spectrometry (see FIG. 4K-1 for Lipid 11 NMR spectrum, FIG. 4K-2 for Lipid 11 LC-MS, and Table 5 for product mass).

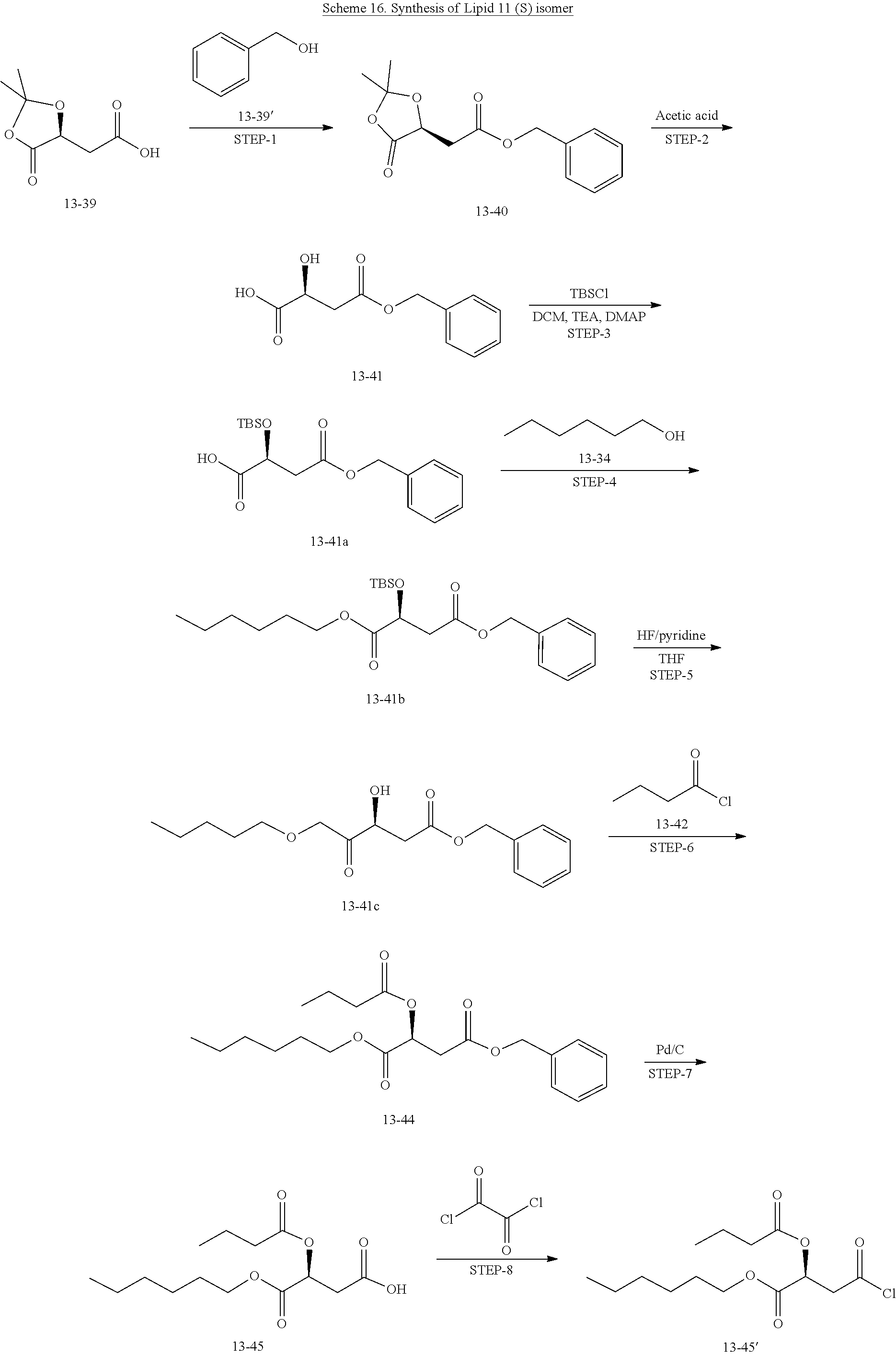

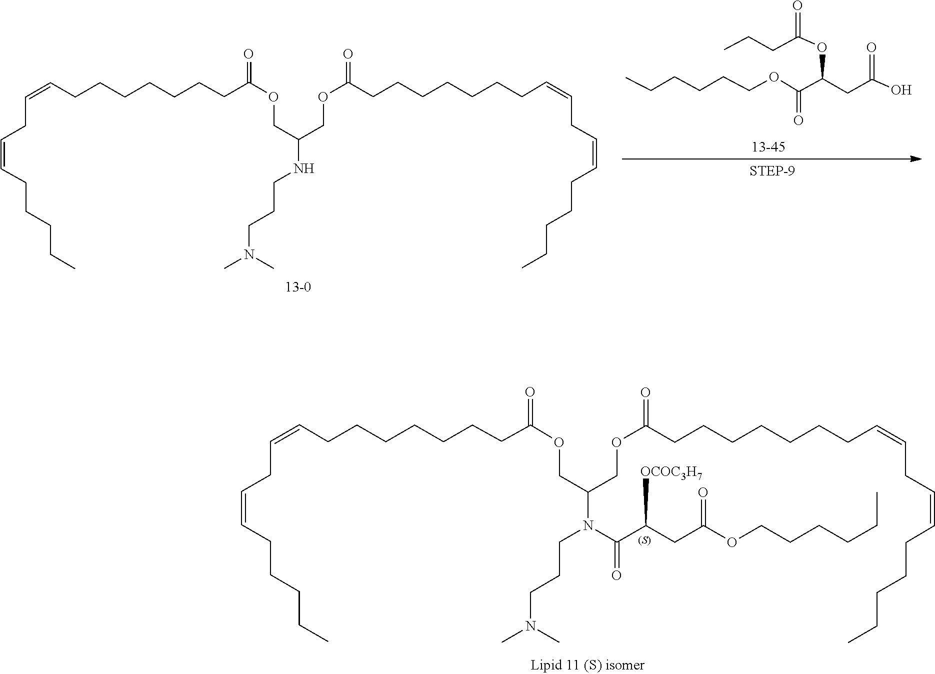

Lipid 11 as a racemic mixture was synthesized similarly as provided in scheme 16-2 below.

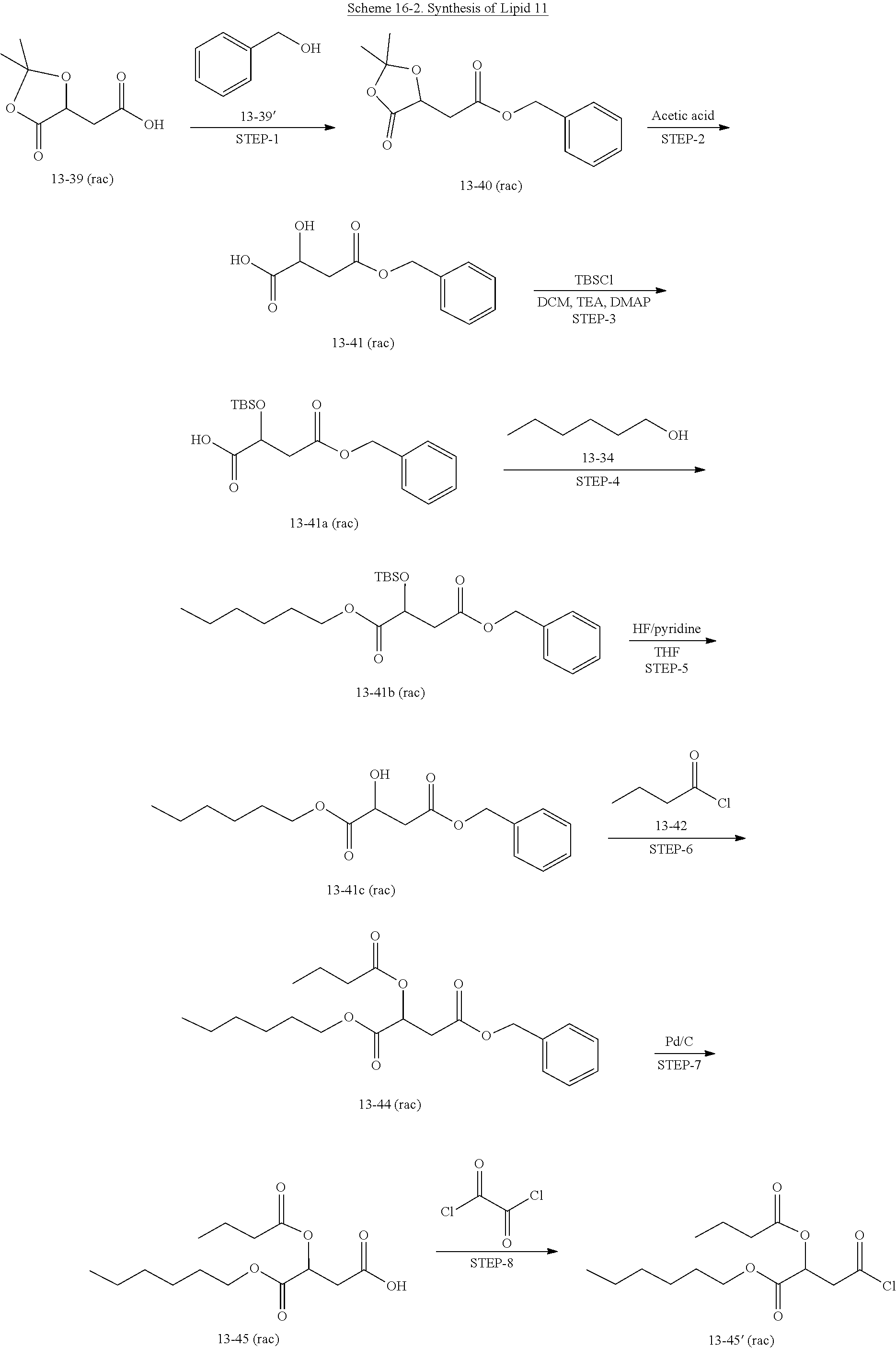

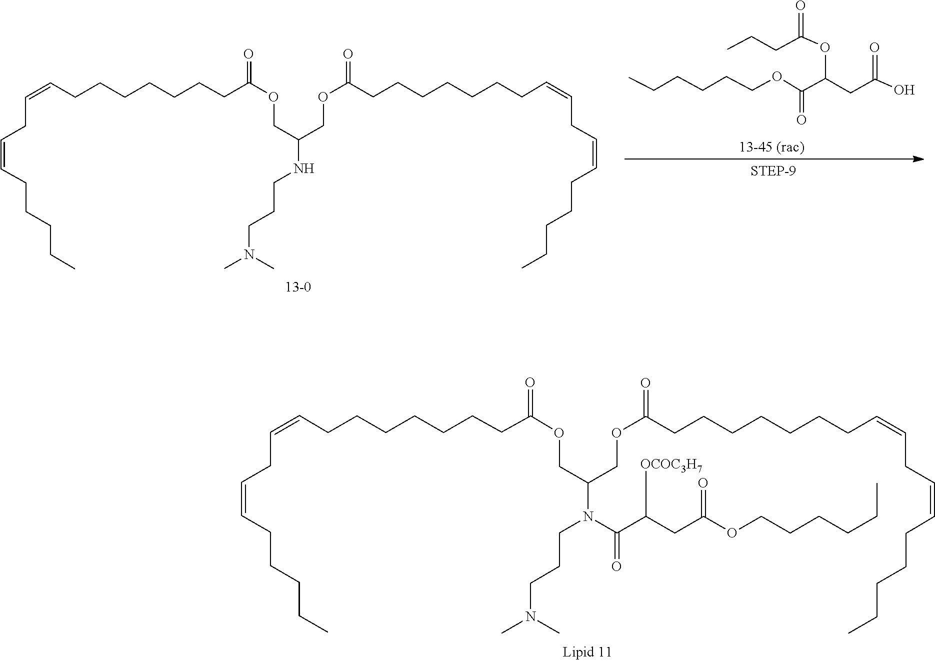

### Synthesis of Lipid 12

Lipid 12 was synthesized as provided in scheme 34 below and as follows. Starting material, 14-3 (3 g, 1.0 eq, 22.37 mmol) was selectively protected in trifluoroacetic anhydride (11.27 g, 2.4 eq, 53.69 mmol) and Benzyl alcohol (15 mL) at room temperature, overnight yielding intermediate 14-4. Crude product was purified by column chromatography (1×) to obtain 4.7 g (96%) purified 14-4. Subsequent acylation of 14-4 (1.0 eq, 4.44 mmol) with n-butanol, 13-34 (4.55 g, 10.0 eq, 44.60 mmol) using EDCI (1.71 g, 2 eq, 8.92 mmol) and DMAP (1.089 g, 2 eq, 8.92 mmol) in 10 mL DCM at RT, overnight yielded 14-5. Crude product was purified by column chromatography (1×) to obtain 800 mg (58%) purified 14-5. Acylation of the free hydroxyl of 14-5 (800 mg, 1.0 eq, 2.59 mmol) with hexanoyl chloride (1.39 g, 4.0 eq, 10.37 mmol) using TEA (1.31 g, 5 eq, 12.97 mmol) and DMAP (10 mg, catalytic amount) in 10 mL toluene at room temperature, overnight yielded intermediate 14-7. Purification of crude product by column chromatography (1×) yielded 470 mg (46%) purified 14-7. Intermediate 14-7 (470 mg, 1 eq., 3.4 mmol) yielded 340 mg (93%) of free acid 14-8. Crude 14-8 (56 mg, 1 eq., 0.18 mmol) was converted to the corresponding chloride, 14-8′, using Oxalyl Chloride (50 μL, 3.4 eq, 0.60 mmol) and DMF (0.2 μL, Catalytic amount) in 1 mL Toluene at room temperature, overnight to afford 56 mg of crude chloride 14-8′. N-acylation of 13-0 (42 mg, 1 eq., 0.059 mmol) with 14-8′ (56.0 mg, 3.0 eq, 0.17 mmol) using TEA (39.0 μL, 5.0 eq, 0.29 mmol) and DMAP (10 mg, Catalytic amount) in 3 mL Toluene yielded Lipid 12. Crude product was purified by column chromatography (1×) to obtain pure Lipid 12 (23 mg, 39%) (≥99% purity by LC-ELSD) and characterized by proton NMR and Mass Spectrometry (see FIG. 4L-1 for Lipid 12 NMR spectrum, FIG. 4L-2 for Lipid 12 LC-ELSD chromatogram, and Table 5 for product mass).

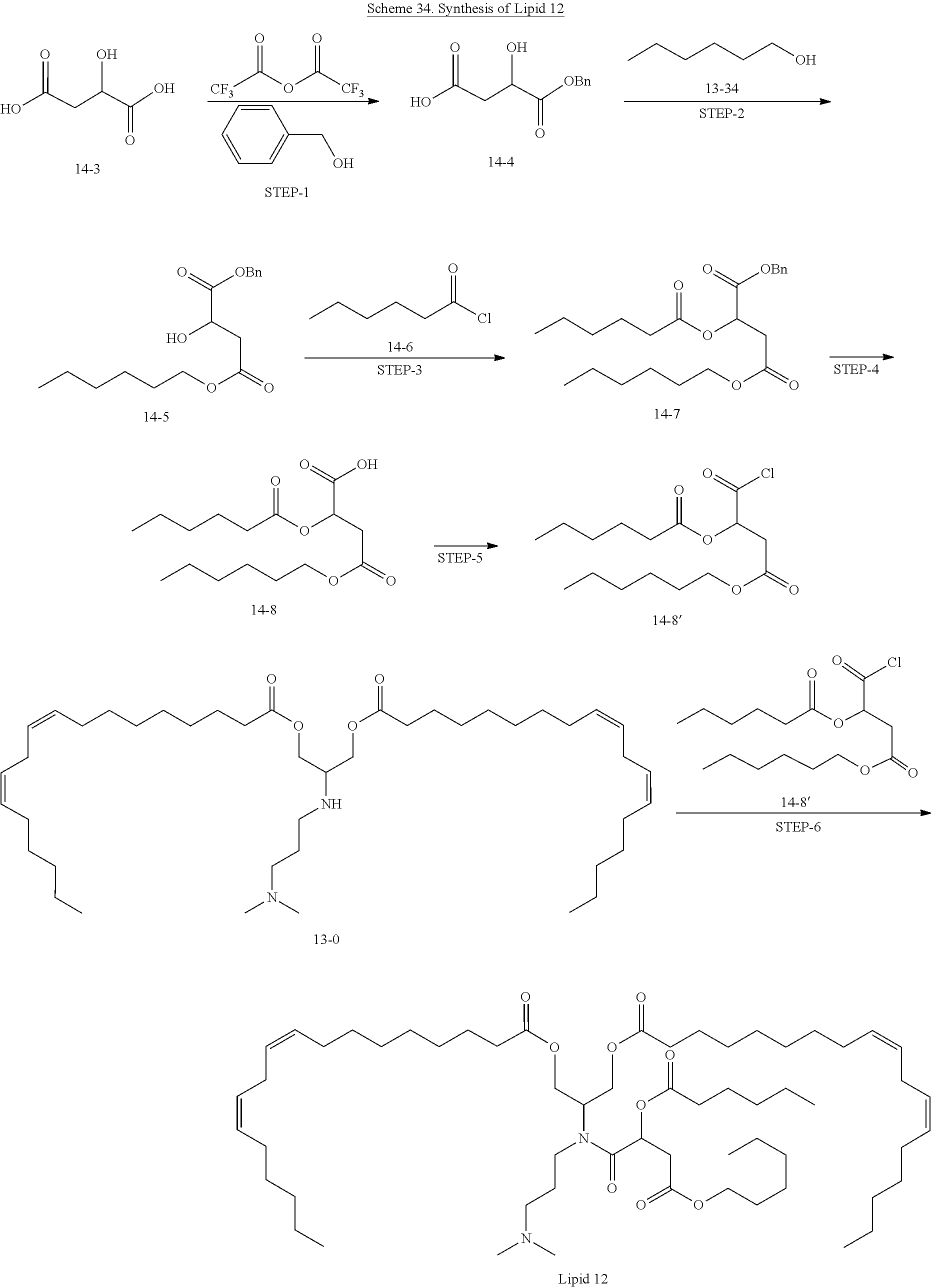

### Synthesis of Lipids 13 by N-Acylation of Intermediate 13-0 Carbodiimide Activation of the Corresponding Carboxylic Acid

### Synthesis of Lipid 13

Lipid 13 was synthesized as provided in scheme 17 below and as follows. Starting material 13-32 (4.8 g, 2.0 eq, 25.0 mmol) was esterified with 1-Butanol (1.13 mL, 1 eq, 12.4 mmol) using EDCI (4.8 g, 2 eq, 25.0 mmol), DIPEA (4.35 mL, 2 eq, 25.0 mmol), and DMAP (280 mg, 0.2 eq, 2.5 mmol) in 20 mL DCM to obtain intermediate 13-33. Crude product was purified by column chromatography to obtain 2.78 g (44%) of pure intermediate 13-33. Intermediate 13-36 was accessed by acylation of n-hexanol (2 g, 1.0 eq, 19.6 mmol) with 2-bromoacetyl bromide, 13-35 (5.05 g, 1.3 eq, 25.0 mmol) using NaHCO₃ (3.95 g, 2.4 eq, 47.0 mmol) in 50 mL acetonitrile. Crude product was purified by column chromatography (1×) to obtain 4.32 g (97%) of pure intermediate 13-36.

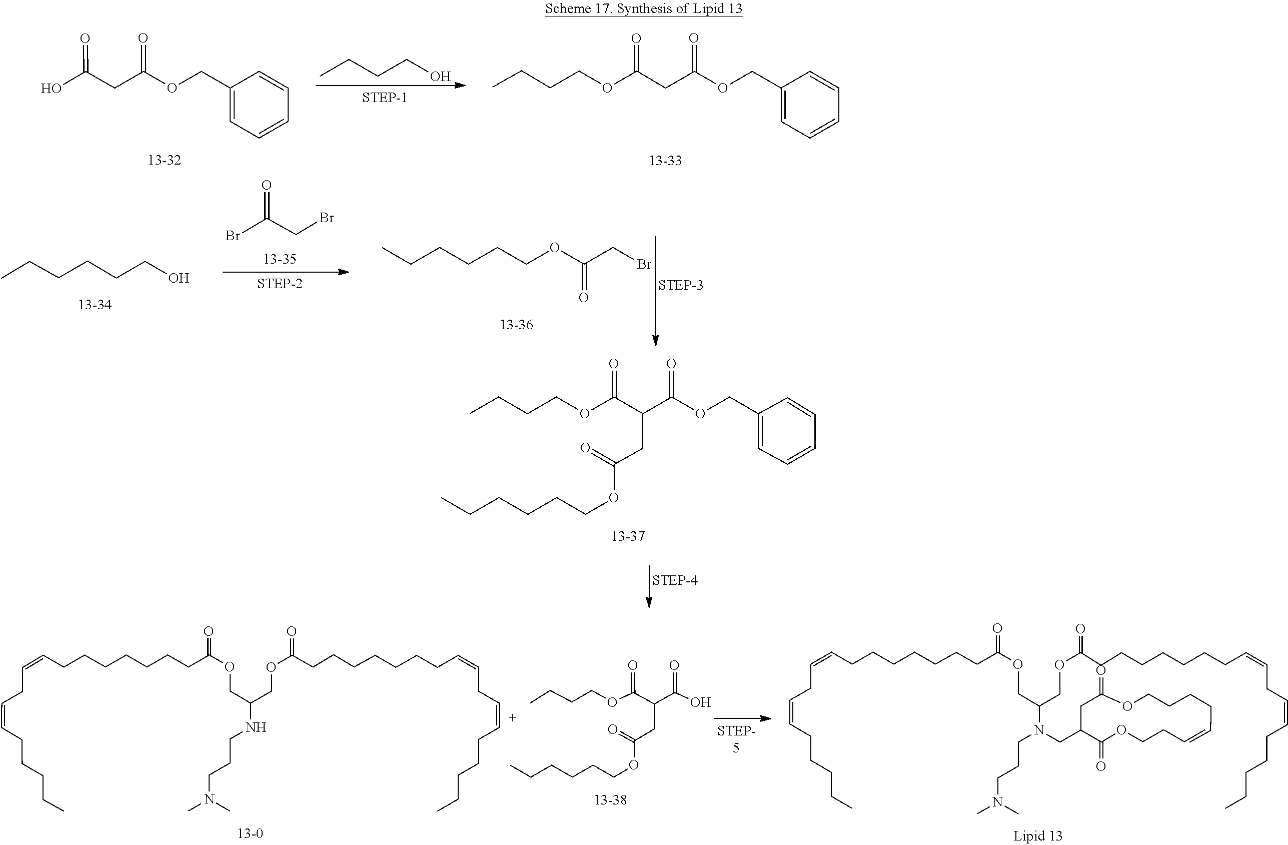

Intermediate 13-37 was accessed by in situ generation of the nucleophilic carbanion of 13-33 (1.25 g, 1.0 eq., 5.0 mmol) using NaH (200 mg, 1.0 eq, 5.0 mmol) in 8 mL DMF and displacement reaction with intermediate 13-36 (1.1 g, 1.0 eq, 5.0 mmol). Crude product was purified by column chromatography (2×) to 1.15 g (58%) of pure intermediate 13-37. Free acid intermediate 13-38 was obtained by deprotection (Pd/C, 230 mg catalyst and hydrogen gas in methanol) of intermediate 13-37 (1.15 g, 1.0 eq, 2.9 mmol). Crude product was purified by column chromatography (4×) to obtain 88 mg (9%) of pure intermediate 13-38. Intermediate 13-0 (105 mg, 1.0 eq, 0.04 mmol) was acylated with 13-38 (2.13 mmol, 0.554 g, 1.1 eq), using DIPEA (78 μL, 3.0 eq, 0.45 mmol), EDCI (87 mg, 3.0 eq, 0.45 mmol), and DMAP (5 mg, 0.3 eq, 0.04 mmol), in 2 mL DCM. Crude product was purified by column chromatography (3×) yielding 41 mg (27%) of pure Lipid 13 (≥99% purity by LC-ELSD) and characterized by proton NMR and Mass Spectrometry (see FIG. 4M-1 for Lipid 13 NMR spectrum, FIG. 4M-2 for Lipid 13 LC-MS, and Table 5 for product mass).

### Synthesis of Lipid 14A by N-Acylation of Intermediate 14-11 Using the Corresponding Acid Chloride

### Synthesis of Lipid 14A

Lipid 14A was synthesized as provided in Scheme 37 below and as follows.

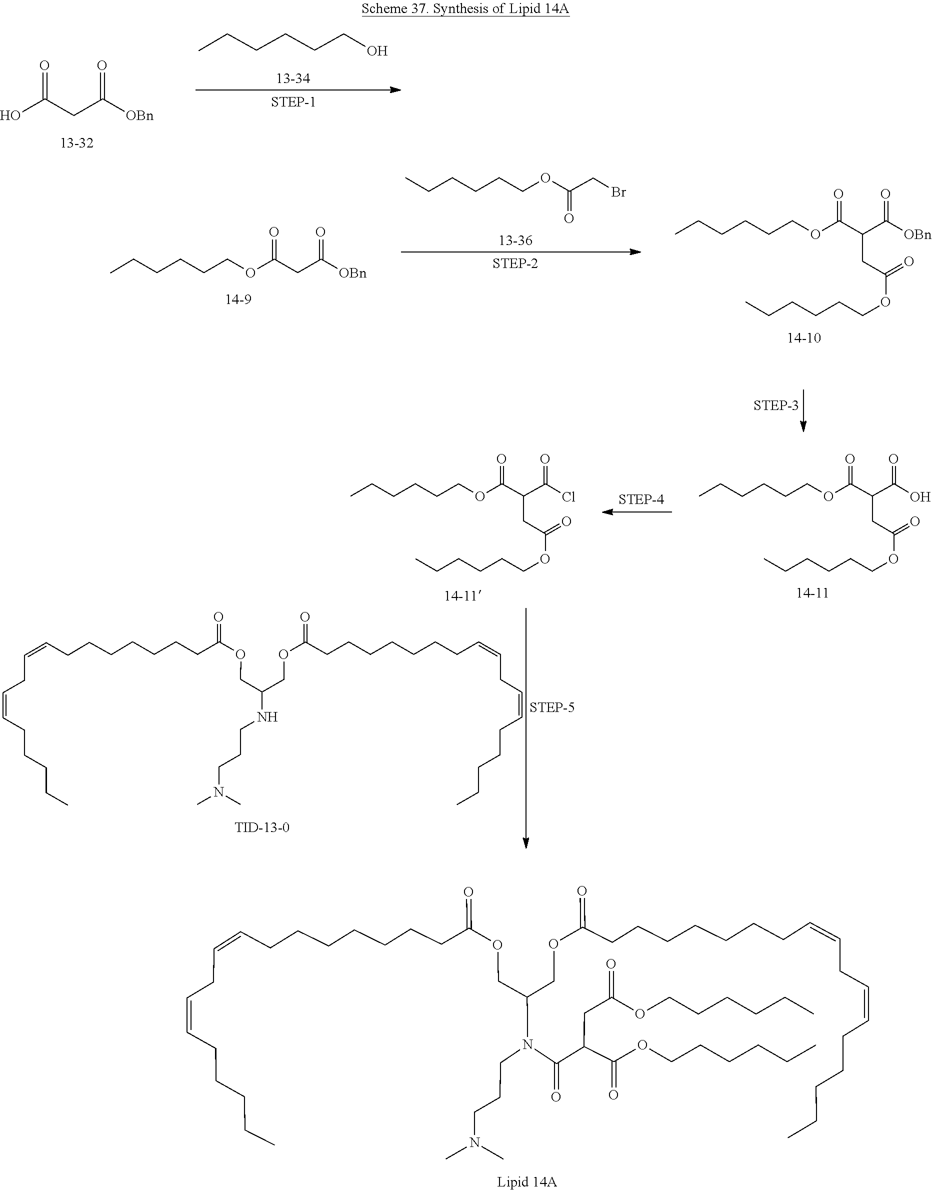

Starting material, mono-benzyl protected malonic acid, 13-32 (9.26 mmol, 1.8 g, 1.0 eq.) was esterified with N-hexanol, 13-34 (92.69 mmol, 9.47 g, 10.0 eq.) using EDCI (18.53 mmol, 3.55 g, 2 eq.) and DMAP (2.26 g, 2 eq, 18.53 mmol) in DCM (20 mL) at room temperature, overnight to obtain intermediate, 14-9 (2.04 g, 79%), Compound 13-36 was prepared by reacting bromoacetyl bromide (38.17 mmol, 7.70 g, 1.3 eq.) with 3.0 g of N-hexanol, 13-34 (1.0 eq, 29.36 mmol) using NaHCO₃ (5.9 g, 2.4 eq, 70.47 mmol) in 30 mL of acetonitrile (0° C. to room temperature) overnight to obtain 6.0 g (91.6%) of intermediate 13-36. Intermediate 14-9 (7.2 mmol, 2.03 g, 1.0 eq.) was converted to the corresponding carbanion and reacted with bromoacetyl bromide (7.70 g, 1.3 eq, 38.17 mmol) using NaHCO₃ (5.9 g, 2.4 eq, 70.47 mmol) in 30 mL of acetonitrile (0° C. to room temperature, overnight) to obtain Intermediate, 14-10 (1.15 g, 38%).

Deprotection of Intermediate 14-10 (3.4 mmol, 1.15 g, 1.0 eq.) by hydrogenation (Pd/C, H₂, RT, overnight) in ethyl acetate yielded Intermediate 14-11 (850 mg, 94%). 14-11 (300 mg, 0.9 mmol, 1.0 eq.) was converted to the corresponding chloride, 14-11′ using Oxalyl Chloride (3.0 mmol, 260 μL, 3.4 eq) in 4 mL Toluene using DMF (0.2 μL, Catalytic quantity) at room temperature for 2 hours. Crude 14-11′ (0.17 mmol, 280 mg, 3.0 eq.) was used for N-acylation of Intermediate 13-0 (0.059 mmol, 200 mg, 1.0 eq.) using TEA (39.0 μL, 5.0 eq, 0.29 mmol), DMAP (10 mg, Catalytic quantity) in 3 mL of Toluene to obtain Lipid 14A. Purification of crude product by column chromatography (DCM: 10% MeOH in DCM) yielded 220 mg (76%) of purified Lipid 14A (98% by HPLC-CAD) and characterized by proton NMR and mass spectrometry (FIG. 4V-1 and FIG. 4V-2 for proton NMR and HPLC-CAD and Table 5 for Mass Spectrometry data).

### Synthesis of Lipid 15 by N-Acylation of Intermediate 13-11a Using the Corresponding Acid Chloride

### Synthesis of Lipid 15

Lipid 15 was synthesized as provided in scheme 18 below and as follows. Starting material, decan-4-ol, 13-29 (10.0 g, 63.0 mmol) was acylated with succinic acid, 13-30 (12.6 g, 126 mmol, 2.0 eq) using DMAP (7.7 g, 63 mmol, 1 eq) and pyridine (5.0 ml) in 5 mL THF and 15 mL DCM to obtain intermediate 13-31. Crude product was purified by column chromatography (3×) to obtain 8.9 g (55%) of pure acid intermediate 13-31. Intermediate 13-31 (1.26 g, 4.9 mmol) was converted to the acid chloride intermediate 13-31′ using oxalyl chloride (1.43 mL, 3.4 eq, 16.66 mmol) and DMF (50 μL, catalytic quantity), in 5 mL Benzene. Product showed only one spot on TLC (as methyl ester) and was used without further purification for acylation (step 3) of intermediate 13-11b. Intermediate 13-11b (275 mg, 0.39 mmol) was acylated with crude acid chloride, 13-31′ (324 mg, 3.0 eq, 1.17 mmol), TEA (270 μL, 5.0 eq, 1.95 mmol), DMAP (20 mg, catalytic quantity) in 10 mL of benzene to obtain Lipid 15. Crude product was purified by column chromatography (2×) to yield 230 mg g (64%) of pure Lipid 15 (99% purity by LC-ELSD) and characterized by proton NMR and Mass Spectrometry (see FIG. 4N-1 for Lipid 15 NMR spectrum, FIG. 4N-2 for Lipid 15 LC-MS, and Table 5 for product mass).

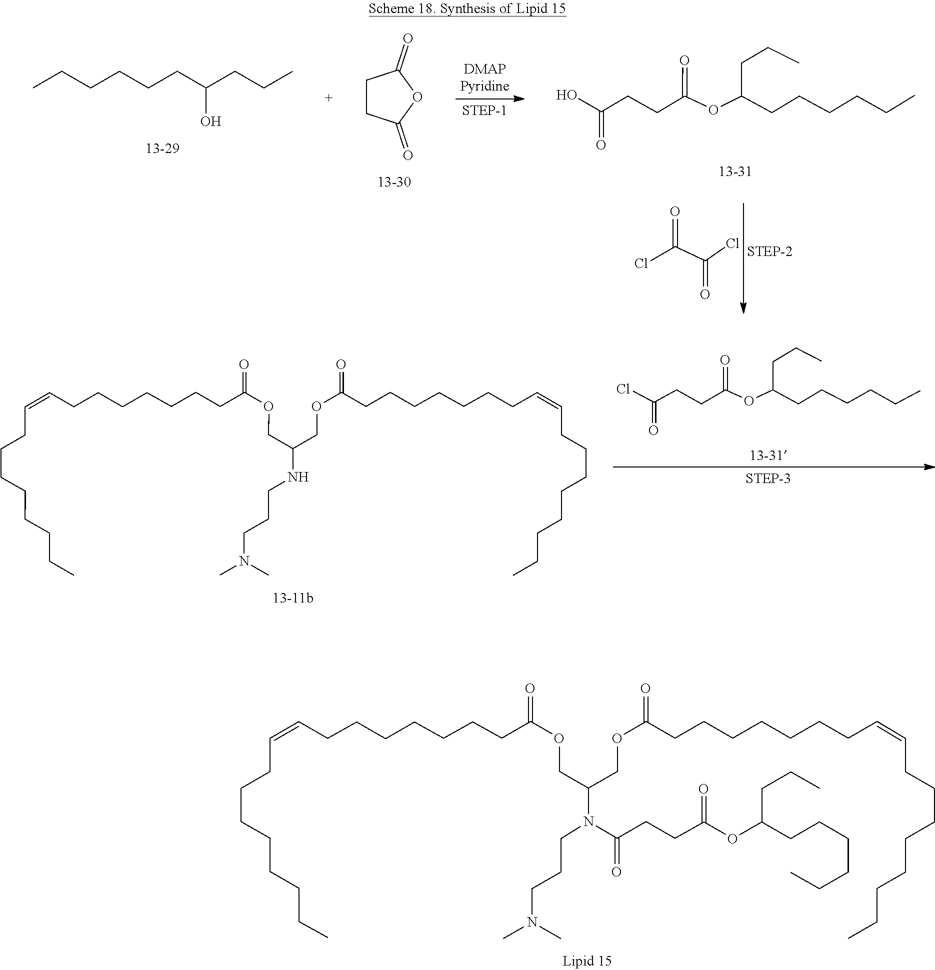

### Synthesis of Lipid 16

Lipid 16 was synthesized as provided in scheme 19 below and as follows. Starting material, octan-3-ol, 13-48 rac (3 g, 23 mmol) was acylated with succinic acid, 13-30 (46.08 mmol, 4.61 g, 2.0 eq) using DMAP (23.04 mmol, 2.8 g, 1.0 eq,) and pyridine (5.0 ml) in 5 mL THF and 15 mL DCM to obtain intermediate 13-31. Crude product was purified by column chromatography (1×) to obtain 3.4 g (64%) of pure acid intermediate 13-47 rac. Intermediate 13-47 rac (300 mg, 0.42 mmol) was converted to the acid chloride intermediate 13-47′ rac using oxalyl chloride (0.38 mL, 4.4 mmol, 3.4 eq.) and DMF (2 μL, catalytic quantity). Product showed only one spot on TLC (as methyl ester) and was used without further purification for acylation (step 3) of intermediate 13-11b. Intermediate 13-11b (270 mg, 0.38 mmol) was acylated with crude acid chloride, 13-47′ rac (0.42 mmol, 300 mg, 3.0 eq.), TEA (260 μL, 5.0 eq, 1.9 mmol), DMAP (20 mg, catalytic quantity) in 5 mL of toluene to obtain Lipid 16. Crude product was purified by column chromatography (1×) to yield 165 mg (47%) of pure Lipid 16 (99% purity by LC-ELSD) and characterized by proton NMR and Mass Spectrometry (see FIG. 40-1 for Lipid 16 NMR spectrum, FIG. 40-2 for Lipid 16 LC-MS, and Table 5 for product mass).

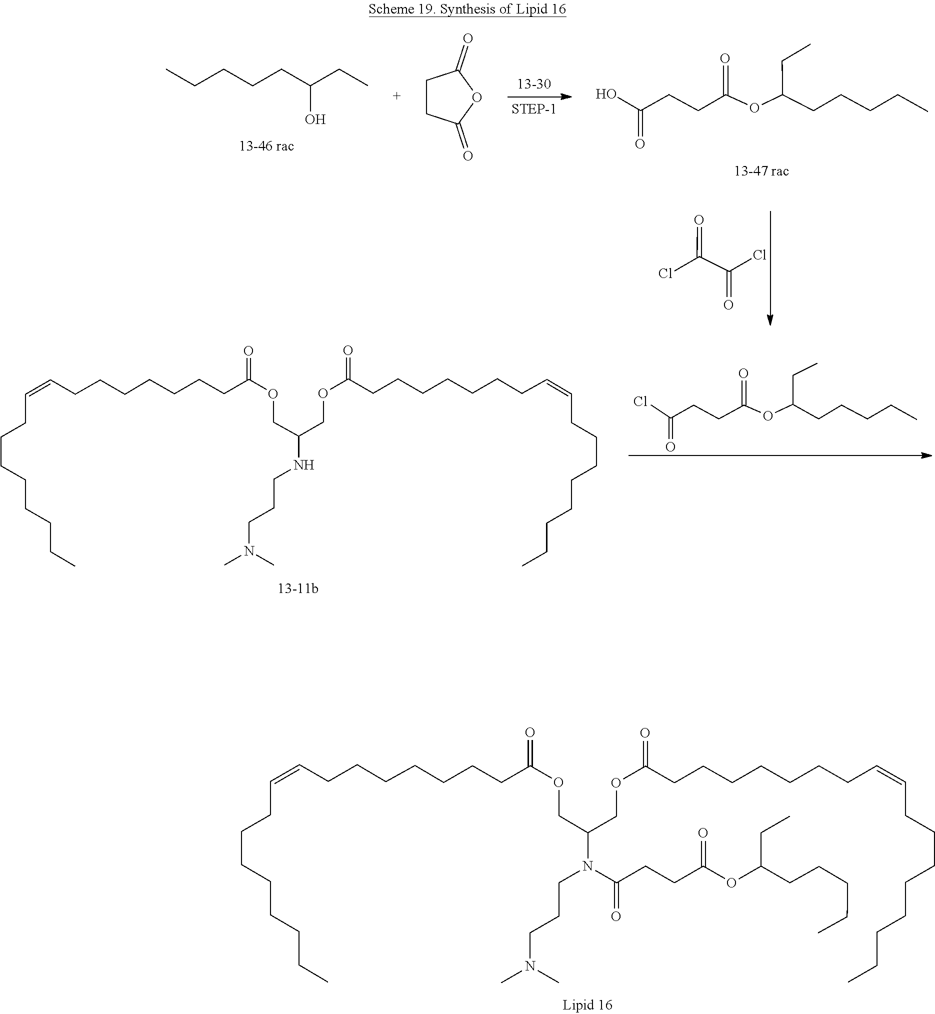

### Synthesis of Lipid 17

Lipid 17 was synthesized as provided in scheme 20 below. Octanedioic acid, 13-51 (5.0 g, 2.0 eq, 28.5 mmol) was mono-acylated with decane-3-ol, 13-29 (2.75 mL, 1.0 eq, 14.3 mmol) using EDCI (3.29 g, 1.2 eq, 17.2 mmol), DMAP (160 mg, 0.12 eq, 1.72 mmol) and TEA (9.96 mL, 5.0 eq, 71.5 mmol) in 50 mL of DCM/DMF (1:1 v/v) (50 mL) at room temperature overnight to obtain free acid 13-53. Crude product was purified by column chromatography (1×) to obtain 1.06 g (28%) of purified 13-53. Acid 13-53 (1.06 g, 2 eq., 3.7 mmol) was reacted with dihydroxyacetone (152 mg, 1.0 eq, 1.7 mmol) using EDCI (816 mg, 2.5 eq, 4.25 mmol), DMAP (50 mg, 0.25 eq, 0.43 mmol) and DIPEA (740 μL, 2.5 eq, 4.3 mmol) in 15 mL DCM at room temperature overnight to obtain ketone 13-54. Crude product was purified by column chromatography (1×) to obtain 890 mg (69%) of purified 13-54. Reductive amination of 13-54 (890 mg, 1.0 eq, 1.3 mmol) with amine, 15-3 (327 μl, 2.0 eq, 2.6 mmol) using acetic acid (150 μL, 2.0 eq, 2.6 mmol) and sodium borohydride triacetate, Na(OAc)₃BH (331 mg, 1.2 eq, 1.5 mmol) in 20 mL DCM (20 ml) at room temperature for 3 hours yielded intermediate 13-55. Crude product was purified by column chromatography (1×) to obtain purified 13-55 (470 mg, 47%). N-acylation of intermediate 13-55 using acid 13-31 and reaction conditions reported for N-acylation of Lipid 15 synthesis resulted in Lipid 17.

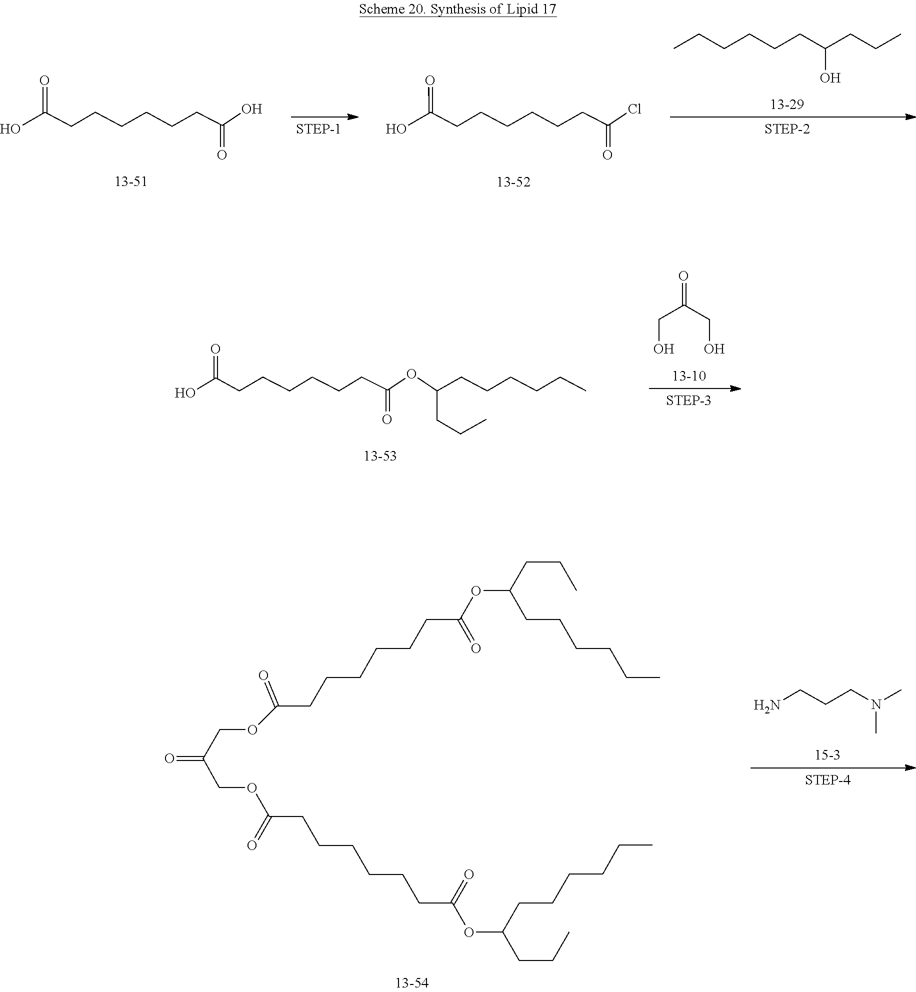

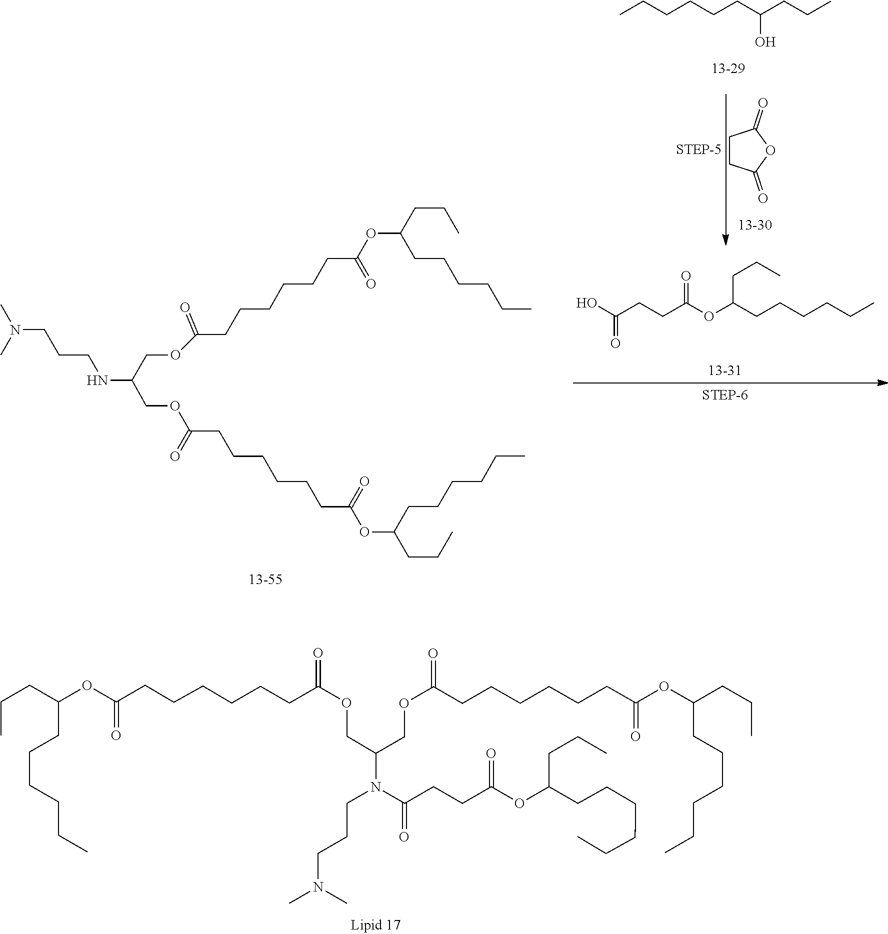 [SMILES: CCCCCCC(O)CCC ; O=C1CCC(=O)O1 ; CCCCCCC(CCC)OC(=O)CCCCCCC(=O)OCC(COC(=O)CCCCCCC(=O)OC(CCC)CCCCCC)NCCCN(C)C ; CCCCCCC(CCC)OC(=O)CCC(=O)O ; CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

### Synthesis of Lipid 17A

Starting material 4-hydroxydecanol (63.1 mmol, 10 g, 1 eq.) was acylated with succinic anhydride, 13-30 (12.64 g, 2.0 eq, 12.2 mmol) using EDCI (2.65 g, 2.5 eq, 126.3 mmol), DMAP (7.7 g, 1.0 eq, 63.1 mmol) and Pyridine (17 mL) in a mixture of 17 ml of THF and 50 mL of DCM at room temperature, overnight to obtain 8.8 g (54%) of acid intermediate, 13-31. Starting material, 1,3-dihydroxyacetone, 13-10 (8.32 mmol, 0.75 g, 1 eq.) was diacylated with intermediate 13-31 (20.8 mmol, 5.36 g, 2.5 eq,) using EDCI (3.98 g, 2.5 eq, 20.8 mmol) and DMAP (0.203 g, 0.2 eq, 1.6 mmol) in 20 mL of DCM at RT, overnight to obtain 1.89 g (40%) of ketone intermediate 13-70. Intermediate 13-70 (3.2 mmol, 31.87 g, 1 eq.) was converted to diamine intermediate 13-71 by reductive amination with N,N-dimethylamino-3-aminopropane, 15-3 (6.5 mmol, 0.66 g, 2.0 eq.) using Na(OAc)₃BH (1.38 g, 2.0 eq, 6.5 mmol), acetic acid (0.37 mL, 2.0 eq, 6.5 mmol) in 15 mL of DCM (15 mL) at room temperature for 3 hours. Purification of crude product by 2× column chromatography (10% MeOH in DCM) yielded 500 mg (23%) of purified Intermediate 13-71. Additional quantity of acid Intermediate 13-31 (19.4 mmol, 0.24 g, 1 eq.) was converted to the corresponding acid chloride 13-31′ using Oxalyl chloride (0.92 mmol, 0.26 mL, 3.4 eq.) and DMF (20 μL, catalytic quantity) in 3 mL of Toluene at room temperature for 2 hours and crude acid chloride 13-31 (0.9 mmol, 0.23 g, 3 eq.) was used for N-acylation of diamine intermediate 13-71 (0.3 mmol, 0.2 g, 1 eq.) using TEA (1.5 mmol, 3.98 g, 5.0 eq,) and DMAP (20 mg, catalytic quantity) in 4 mL of Toluene at room temperature overnight to yield Lipid 17A. Crude product was purified 2× by column chromatography (DCM: 10% MeOH in DCM) to obtain 165 mg (60%) of pure Lipid 17A (>99% by HPLC-CAD) and characterized by proton NMR and mass spectrometry (FIG. 4W-1 and FIG. 4W-2 for proton NMR and HPLC-CAD and Table 5 for Mass Spectrometry data).

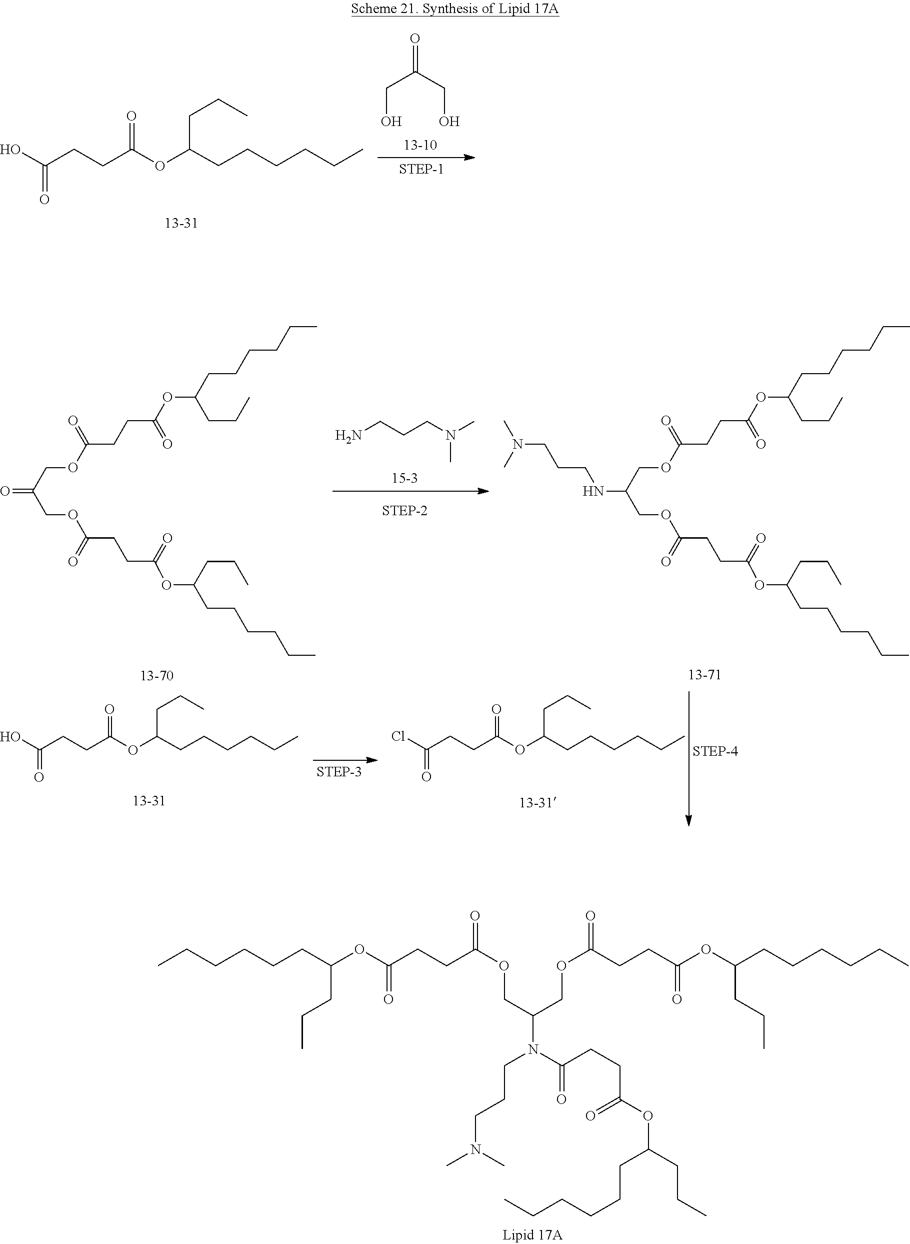

### Synthesis of Lipid 18 and its Isomer

An isomer of lipid 18 was synthesized as provided in scheme 21-1 below. Lipid 18 was synthesis using methods analogous to those reported for Lipid 17 by replacing decan-3-ol with octane-2-ol in the Step 1.

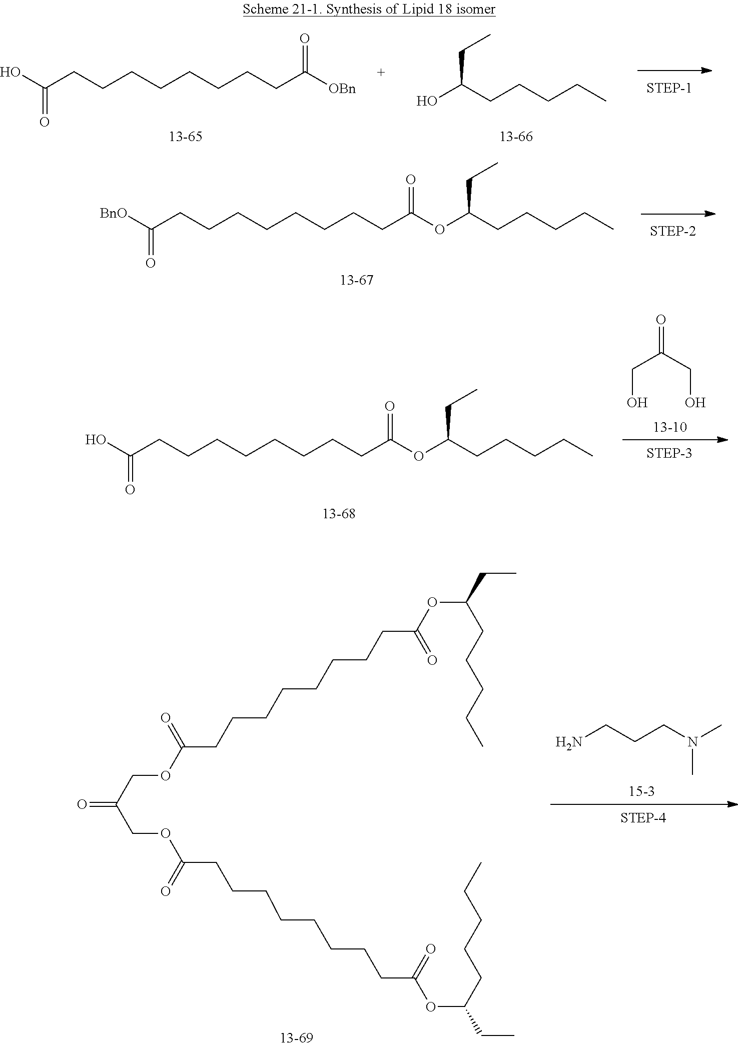

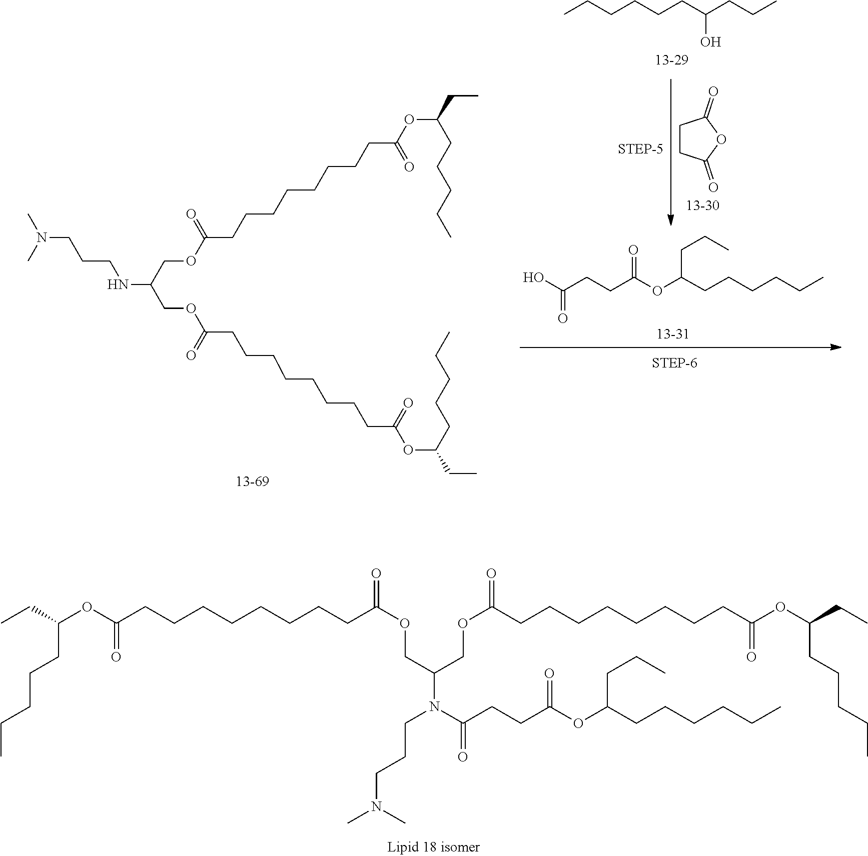 [SMILES: CCCCCCC(O)CCC ; CCCCC[C@H](CC)OC(=O)CCCCCCCCC(=O)OCC(COC(=O)CCCCCCCCC(=O)O[C@@H](CC)CCCCC)NCCCN(C)C ; CCCCCCC(CCC)OC(=O)CCC(=O)O ; O=C1CCC(=O)O1 ; CCCCCCC(CCC)OC(=O)CCC(=O)N(CCCN(C)C)C(COC(=O)CCCCCCCCC(=O)O[C@@H](CC)CCCCC)COC(=O)CCCCCCCCC(=O)O[C@@H](CC)CCCCC]

Lipid 18 as a racemic mixture was synthesized as provided in scheme 21-2 below.

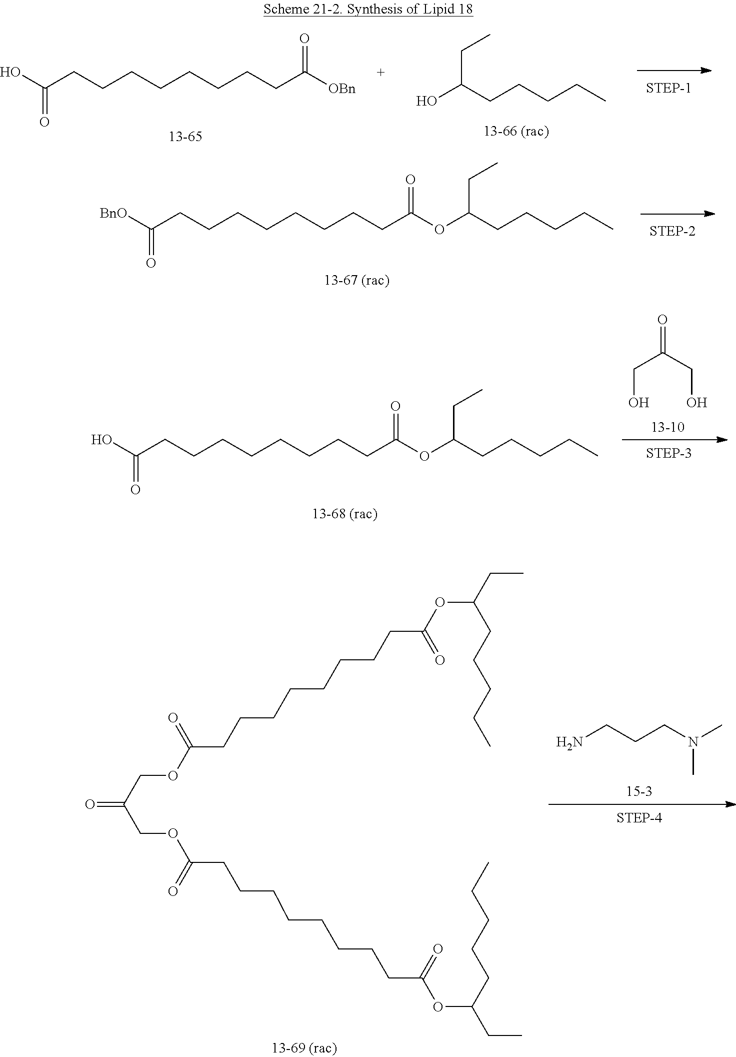

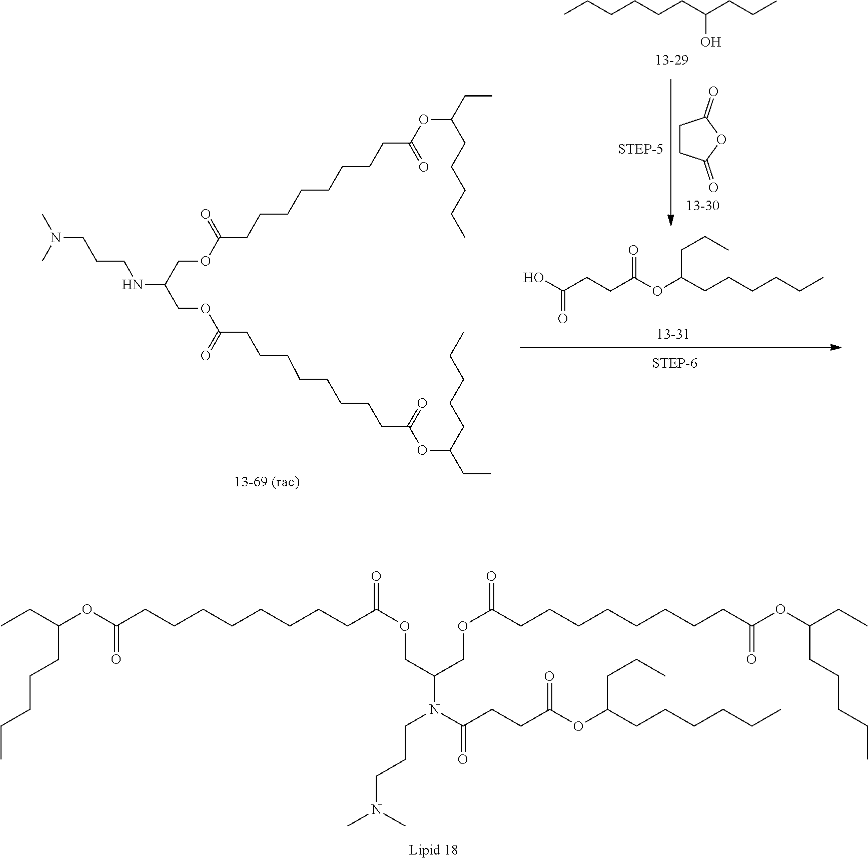 [SMILES: CCCCCCC(O)CCC ; CCCCCC(CC)OC(=O)CCCCCCCCC(=O)OCC(COC(=O)CCCCCCCCC(=O)OC(CC)CCCCC)NCCCN(C)C ; CCCCCCC(CCC)OC(=O)CCC(=O)O ; O=C1CCC(=O)O1 ; CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC]

### Synthesis of Lipid 18A

Starting material 4-hydroxydecanol, 13-29 (31.6 mmol, 5 g, 1 eq.) was acylated with adipic acid, 13-72 (10.4 g, 2.0 eq, 63.2 mmol) using EDCI (37.90 mmol, 7.3 g, 1.2 eq,), DMAP (0.5 g, 0.12 eq, 3.8 mmol, 0.5 g, 0.12 eq,) and TEA (158 mmol, 22 mL, 5.0 eq.) in a mixture of 50 mL of DCM (50 mL) 50 mL DMF (50 mL) at room temperature, RT, overnight to obtain 9.8 g (90%) of acid intermediate, 13-74. Starting material, 1,3-dihydroxyacetone, 13-10 (11.09 mmol, 1.0 g, 1.0 eq.) was diacylated with acid Intermediate 13-74 (27.7 mmol, 7.93 g, 2.5 eq.) using EDCI (27.7 mmol, 5.32 g, 2.5 eq.) and DMAP (3.58 g, 0.2 eq, 2.2 mmol) in 30 mL at room temperature overnight to obtain 1.18 g (17%) of ketone intermediate 13-75. Intermediate 13-75 (3.2 mmol, 1.16 g, 1.0 eq.) was converted to diamine intermediate 13-76 by reductive amination with N,N-dimethylamino-3-aminopropane, 15-3 (6.5 mmol, 0.66 g, 2.0 eq.) using Na(OAc)₃BH (1.38 g, 2.0 eq, 6.5 mmol), acetic acid (0.37 mL, 2.0 eq, 6.5 mmol) in 15 mL of DCM (15 mL) at room temperature for 3-4 hours. Purification of crude product by 2× column chromatography (10% MeOH in DCM) yielded 660 mg (50%) of purified Intermediate 13-76. Additional quantity of acid Intermediate 13-31 (19.4 mmol, 0.24 g, 1 eq.) was converted to the corresponding acid chloride 13-31′ using Oxalyl chloride (0.92 mmol, 0.26 mL, 3.4 eq.) and DMF (20 μL, catalytic quantity) in 3 mL of Toluene at room temperature for 2 hours and crude acid chloride 13-31 (0.9 mmol, 0.23 g, 3 eq.) was used for N-acylation of diamine intermediate 13-76 (0.3 mmol, 0.2 g, 1 eq.) using TEA (1.5 mmol, 3.98 g, 5.0 eq,) and DMAP (20 mg, catalytic quantity) in 4 mL of Toluene at room temperature overnight to yield Lipid 18A. Crude product was purified 2× by column chromatography (DCM: 10% MeOH in DCM) to obtain 175 mg (60%) of pure Lipid 18A (>99% by HPLC-CAD) and characterized by proton NMR and mass spectrometry (FIG. 4X-1 and FIG. 4X-2 for proton NMR and HPLC-CAD and Table 5 for Mass Spectrometry data).

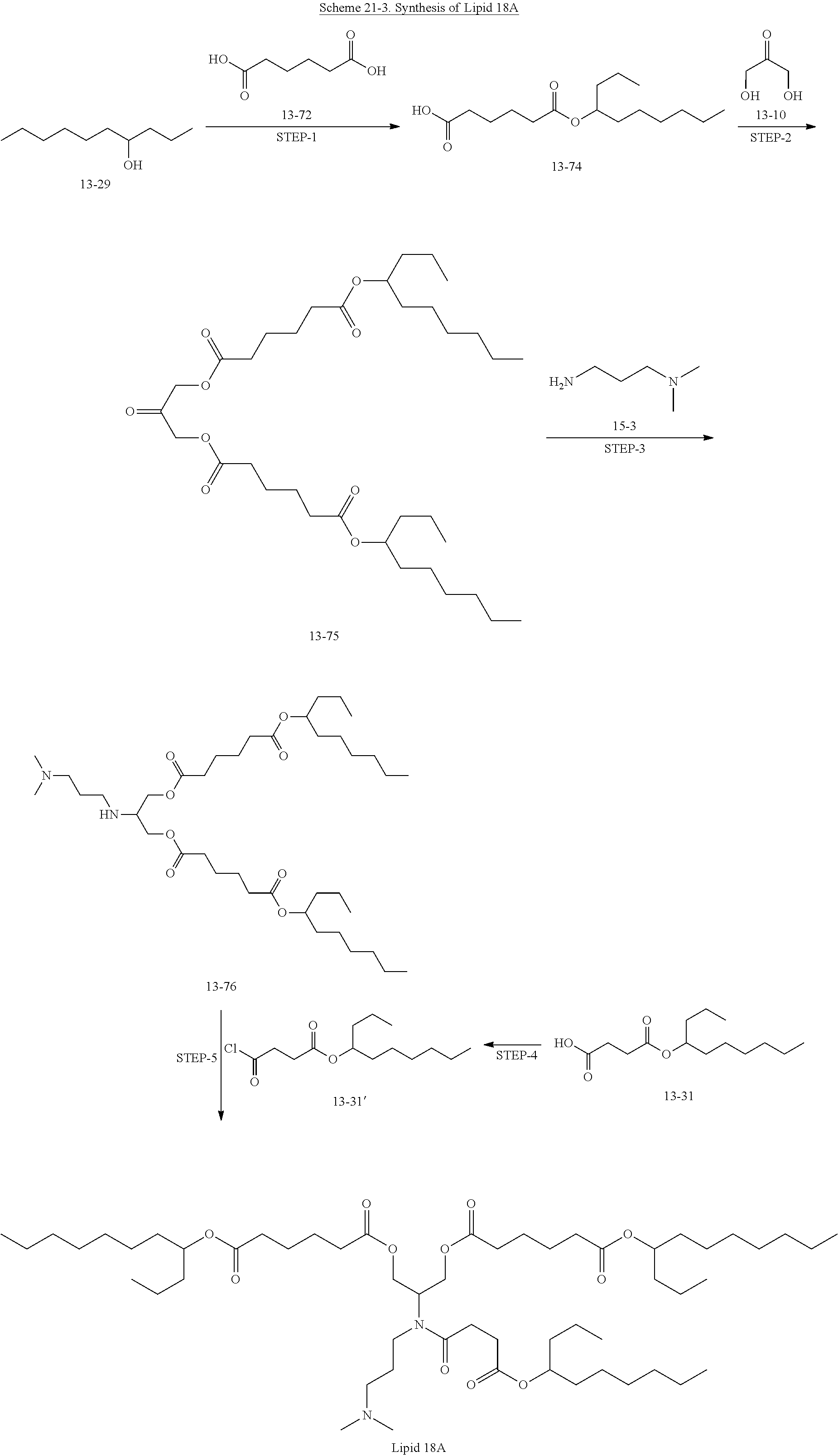

### Synthesis of Lipid 19

Lipid 19 was synthesized as provided in scheme 22 below and as follows. Starting material dihydroxyacetone (422 mg, 4.7 mmol) was acylated with compound 13-56 (3.0 g, 2.5 eq, 11.71 mmol) using EDCI (2.24 g, 2.5 eq, 11.71 mmol), DIPEA (2.0 mL, 2.5 eq, 11.71 mmol), and DMAP (115 mg, 0.2 eq, 0.94 mmol) in 10 mL DCM yielding 2.1 g (79%) of intermediate 13-57. Reductive amination of 13-57 (2.1 g, 1.0 eq, 3.7 mmol) with amine 15-3 (925 μL, 2.0 eq, 7.4 mmol) using acetic acid (430 μL, 2.0 eq, 7.4 mmol), Na(OAc)₃BH (923 mg, 1.2 eq, 4.44 mmol) in 10.0 mL DCM yielding 1.55 g (65%) of intermediate 13-58. Intermediate 13-31 was produced as described in the synthesis of Lipid 9 and Lipid 15 earlier. N-acylation of intermediate 13-58 (484 mg, 1.0 eq, 0.74 mmol) with 13-31 (380 mg, 2.0 eq, 1.48 mmol) using EDCI (291 mg, 2.0 eq, 1.48 mmol), DIPEA (247 μL, 2.0 eq, 1.48 mmol), and DMAP (45 mg, 0.5 eq, 0.37 mmol) in 4.0 mL DCM at room temperature, overnight yielded 423 mg (63%) of pure lipid 19 (>99% purity).

See FIG. 4P-1 for Lipid 19 NMR spectrum, FIG. 4P-2 for Lipid 19 reverse phase LC-ELSD chromatogram, and Table 5 for product mass.

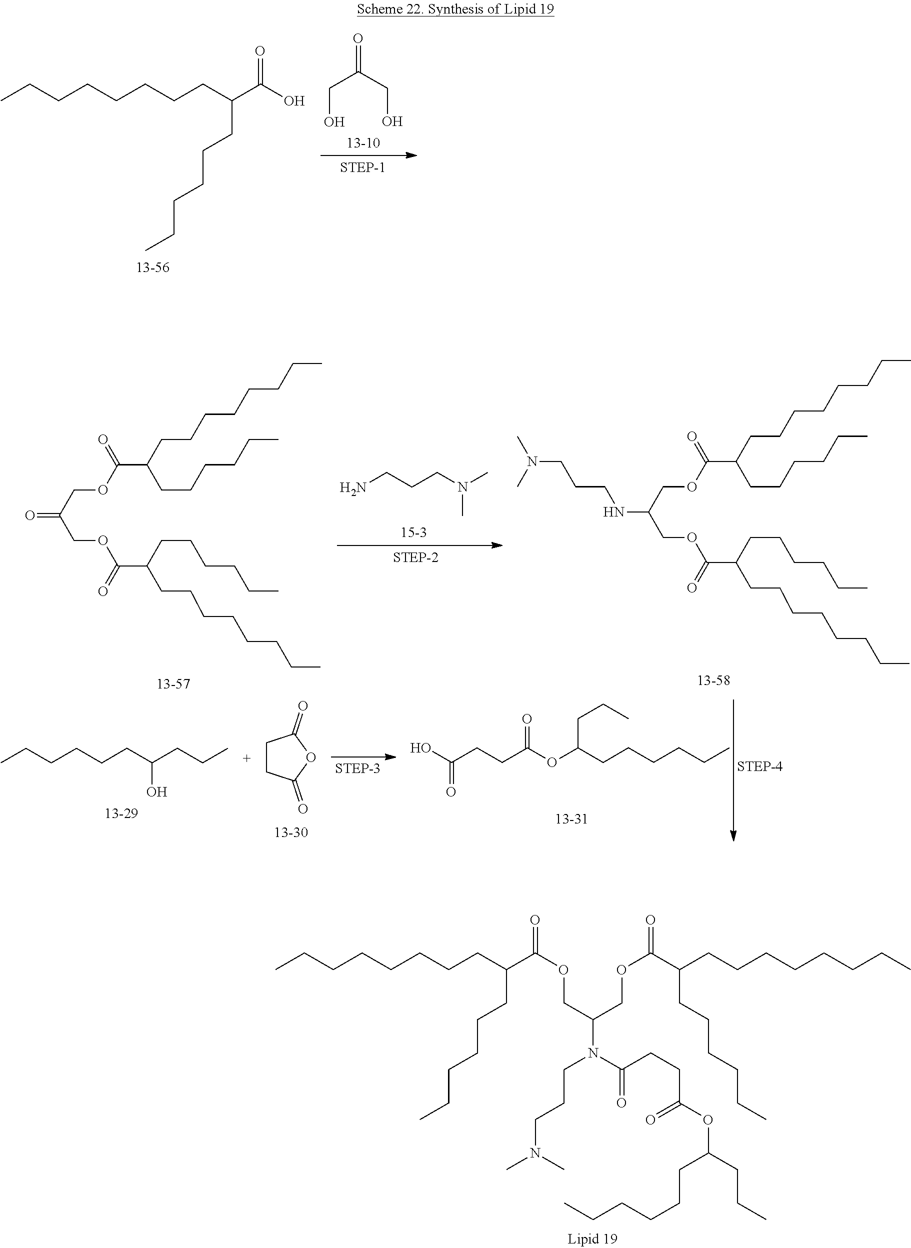

### Synthesis of Lipid 19A

Starting material tertiary-butyl protected suberic acid 13-51 (8.6 mmol, 2.0 g, 1 eq.) was esterified using 4-hydroxydecanol 13-52 (13.02 mmol, 2.06 g, 1.5 eq) using EDCI (13.02 mmol, 2.49 g, 1.5 eq), DMAP (4.3 mmol, 0.53 g, 0.5 eq) and DIPEA (13.02 mmol, 1.68 g, 1.5 eq) in 20 mL of dichloromethane at room temperature for 4 hours. yielding 2.83 g (88%) of protected intermediate 13-53. Intermediate 13-53 (4.05 mmol, 1.5 g, 1.0 eq.) was deprotected using 4N HCl in 10 mL dioxane at room temperature overnight yielding 1.07 g (84%) of acid intermediate 13-54.

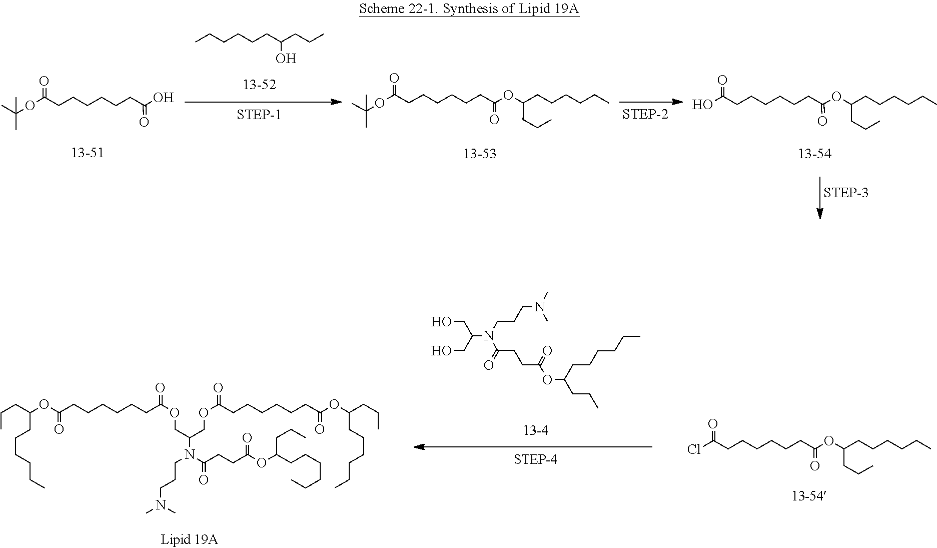

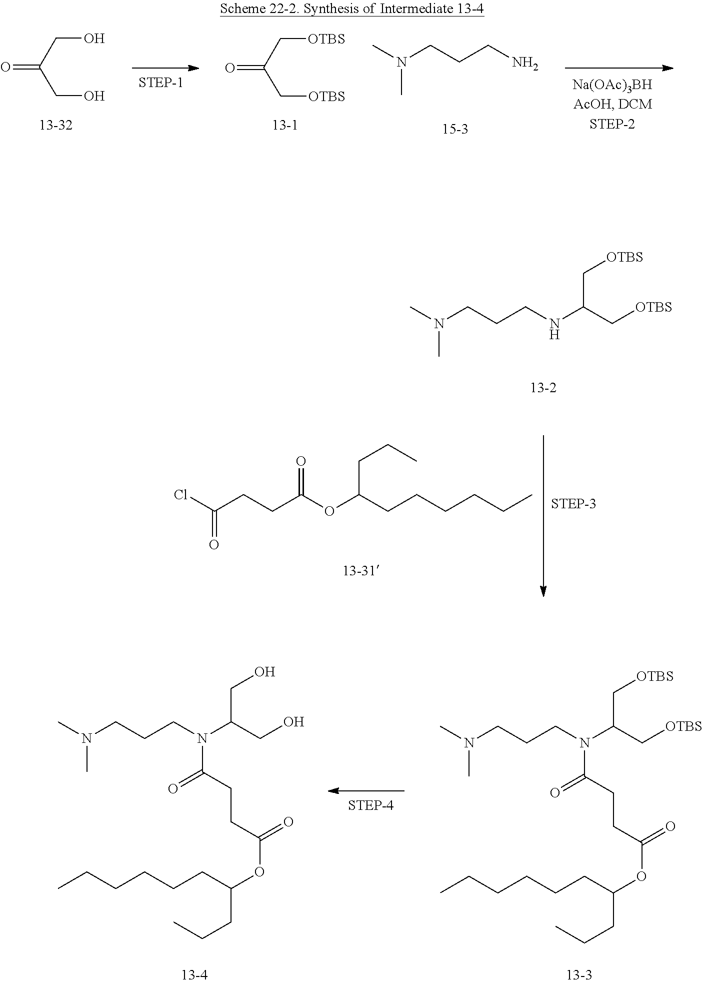

Protection of starting material dihydroxyacetone 13-32 (111 mmol, 10 g, 1 eq.) using tert-butyltrimethylsilyl chloride TBSCl (332 mmol, 50 g, 3.0 eq), TEA (148 mmol, 160 mL, 10.34 eq,) and DMAP (23 mmol, 2.80 g, 0.21 eq.) in 420 mL DCM (420.0 mL) at room temperature overnight yielded protected intermediate 13-1. A second batch of 13-1 was produced from 50 g (0.56 mol, 1.0 eq) of additional 13-32 using TBSCl (1.68 mol, 250 g, 3.0 eq), TEA (5.6 mol, 400 mL, 10.34 eq.), DMAP (0.11 mol, 13.7 g, 0.21 eq,) in 800 mL of DCM at room temperature overnight. Crude products from the two batches were reductively aminated separately and combined prior to purification. First batch of 13-1 (176 g, 552.0 mmol) was reductively aminated using N,N-dimethylaminopropyl amine 15-3, (1104.0 mmol, 139 mL, 2.0 eq.), Acetic acid (1104.0 mmol, 64 mL, 2.0 eq.), and Na(OAc)₃BH (662.0 mmol, 135 g, 1.2 eq.), in 1.5 L of dichloromethane at room temperature for 3 hours. The second batch of 13-1 (36.8 g, 115.4 mmol) was reductively aminated using N,N-dimethylaminopropyl amine 15-3, (230.8 mmol, 29 mL, 2.0 eq.), Acetic acid (230.8 mmol, 13.4 mL, 2.0 eq,), and Na(OAc)₃BH (138.5 mmol 28.2 g, 1.2 eq.), in 300 mL of dichloromethane at room temperature for 3 hours. Combined crude product from the two batches was purified by filter column chromatography on silica column eluting with DCM and (10% MeOH in DCM+1% NH₄OH) to obtain desired product yielding 17 g pure intermediate 13-2 based on TLC.

Two batches of 13-2 were separately deprotected using identical reactions conditions; each consisting of 13-2 (300 mg, 0.465 mmol) in HF-pyridine, (4.65 mmol, 0.42 mL, 10.0 eq.) and 2 mL THF at room temperature for 2 hours.

Two batches of acid intermediate 13-54 were separately converted to the corresponding acid chloride using identical reaction conditions; each consisting of 13-54 (881 mg, 2.8 mmol) using oxalyl chloride (9.5 mmol, 0.8 mL, 3.4 eq,), DMF (100 μL, catalytic quantity) in 6.0 mL of toluene at room temperature for 2 hours.

Crude di-hydroxy intermediate 13-4 (194 mg, 0.465 mmol) and crude acid chloride 13-54′ (2.8 mmol, 881 mg, 6.0 eq.), were combined with TEA (4.65 mmol, 0.65 mL, 10.0 eq,) in 8.0 mL of toluene at room temperature overnight. Crude product was purified on ISCO column chromatography on silica column eluting with DCM and 10% MeOH in DCM. Column purification was repeated after product isolation yielding 247 mg (53%) of 76% pure (HPLC-CAD) Lipid 19A. Product was re-purified on ISCO column chromatography on silica column eluting with DCM and 10% MeOH in DCM. yielding 122 mg of >99% purity (HPLC-CAD) Lipid 19A (see FIG. 4AI-1 and FIG. 4AI-2 for characterization by proton NMR, and LC-CAD purity and Table 5 for Mass Spectrometry data).

### Synthesis of Lipid 20

Lipid 20 was synthesized as provided in scheme 23 below and as follows. Monoprotected succinic acid, 13-59 (2.0 g, 1.0 eq, 9.65 mmol) was reduced to the corresponding alcohol using Borane-dimethyl sulfide (6.2 mL, 7.0 eq, 67.0 mmol) at 0-5° C., 1 hr followed by room temperature reaction overnight. Crude product was purified by column chromatography (2×) yielding 1.3 g (71%) of pure compound 13-60. Intermediate 13-60 (1.3 g, 1.3 eq, 6.7 mmol) was used to acylate acid 13-56 (1.51 mL, 1.0 eq, 5.0 mmol) using EDCI (1.63 g, 1.7 eq, 8.5 mmol), DIPEA (1.48 mL, 1.7 eq, 8.5 mmol) and DMAP (98 mg, 0.17 eq, 0.85 mmol) in 10.0 mL DCM at room temperature overnight. Crude product was purified by column chromatography (1×) yielding 1.88 g (65%) of pure intermediate 13-61. Subsequent deprotection by hydrogenation on Pd/C/Hydrogen gas (400 mg) in methanol yielded 1.42 g of free acid 13-62 (99%) of crude product. Crude 13-62 (1.32 g, 2.2 eq, 4.2 mmol) was used to acylate dihydroxyacetone, 13-10 (172 mg, 1.0 eq, 1.9 mmol), EDCI (958 mg, 2.6 eq, 5.0 mmol), DIPEA (870 μL, 2.6 eq, 5.0 mmol), and DMAP (56 mg, 0.26 eq, 0.5 mmol) in 10.0 mL DCM at room temperature, overnight to obtain ketone 13-63. Crude product was purified by column chromatography to obtain 120 mg (3.8%) of pure 13-63. Reductive amination of 13-63 (120 mg, 1.0 eq, 0.16 mmol) with amine 15-3 (42 μl, 2.0 eq, 0.32 mmol) using acetic acid (18 μL, 2.0 eq, 7.8 mmol) and Na(OAc)₃BH (41 mg, 1.2 eq, 0.19 mmol) in 3 mL DCM at room temperature of 3 hours yielded intermediate 13-64. Crude product was purified by column chromatography (1×) to obtain 23 mg (17%) of purified intermediate 13-64. N-acylation of 13-64 (23 mg, 1.0 eq, 0.028 mmol) with acid 13-31 (8.7 mg, 1.2 eq, 0.034 mmol) using EDCI (6.4 mg, 1.2 eq, 0.034 mmol), DIPEA (5.8 μL, 1.2 eq, 0.034 mmol) and DMAP (1 mg, cat) in 1.5 mL DCM at room temperature overnight yielded Lipid 20. Crude product was purified by column chromatography (1×) to obtain 21 mg (70%) of pure lipid 20 (99%).

See FIG. 4Q-1 for Lipid 20 NMR spectrum, FIG. 4Q-2 for Lipid 20 reverse phase LC-ELSD chromatogram, and Table 5 for product mass.

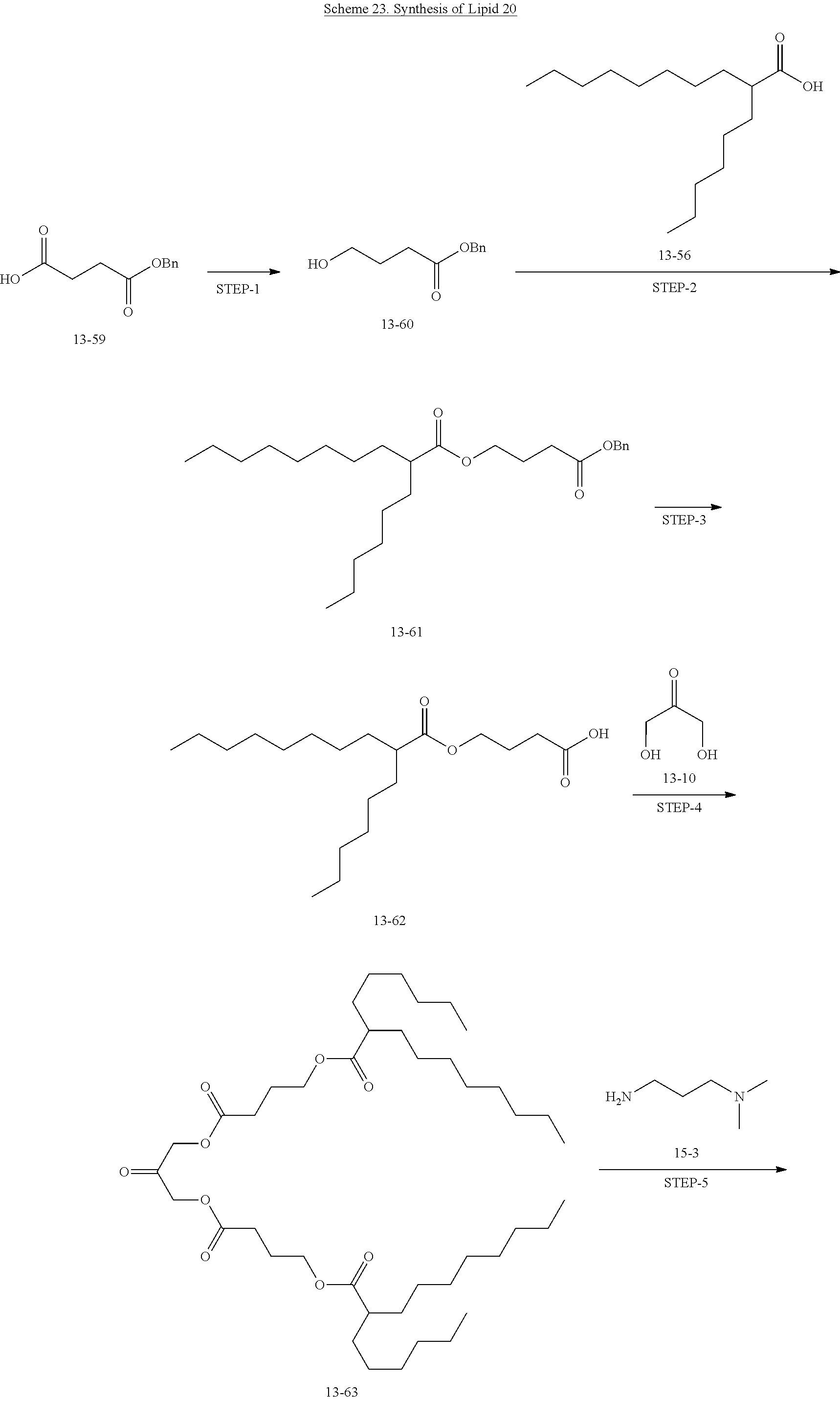

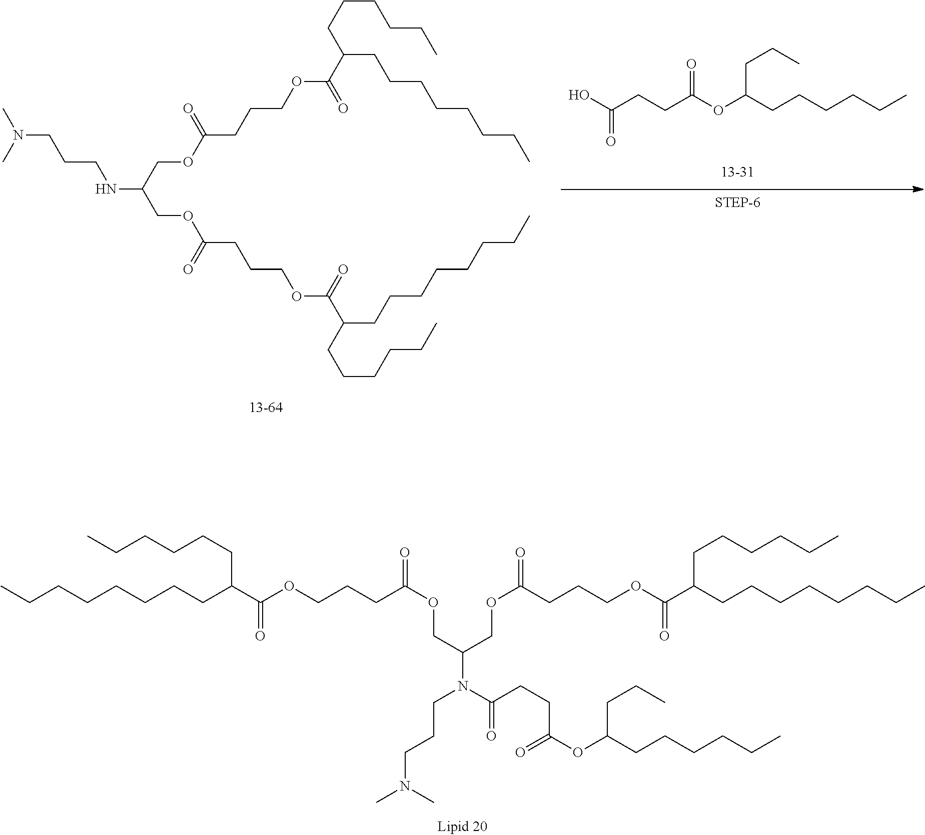 [SMILES: CCCCCCCCC(CCCCCC)C(=O)OCCCC(=O)OCC(COC(=O)CCCOC(=O)C(CCCCCC)CCCCCCCC)NCCCN(C)C ; CCCCCCC(CCC)OC(=O)CCC(=O)O ; CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC]

### Synthesis of Lipid 20A

Starting material tertiary-butyl protected sebacic acid 14-19 (15.5 mmol, 4.0 g, 1 eq.) was esterified using 4-hydroxydecanol 14-20 (23.25 mmol, 3.7 g, 1.5 eq) and EDCI (23.25 mmol, 4.5 g, 1.5 eq), DMAP (7.75 mmol, 0.95 g, 0.5 eq) and DIPEA (23.25 mmol, 4 mL, 1.5 eq.) in 20 mL of dichloromethane at room temperature overnight yielding 4.1 g (66%) of protected intermediate ester 14-21. Intermediate 14-21 (10.3 mmol, 4.1 g, 1.0 eq.) was deprotected using 4N HCl in 15 mL dioxane at room temperature overnight yielding 2.7 g (77%) of acid intermediate 14-22.

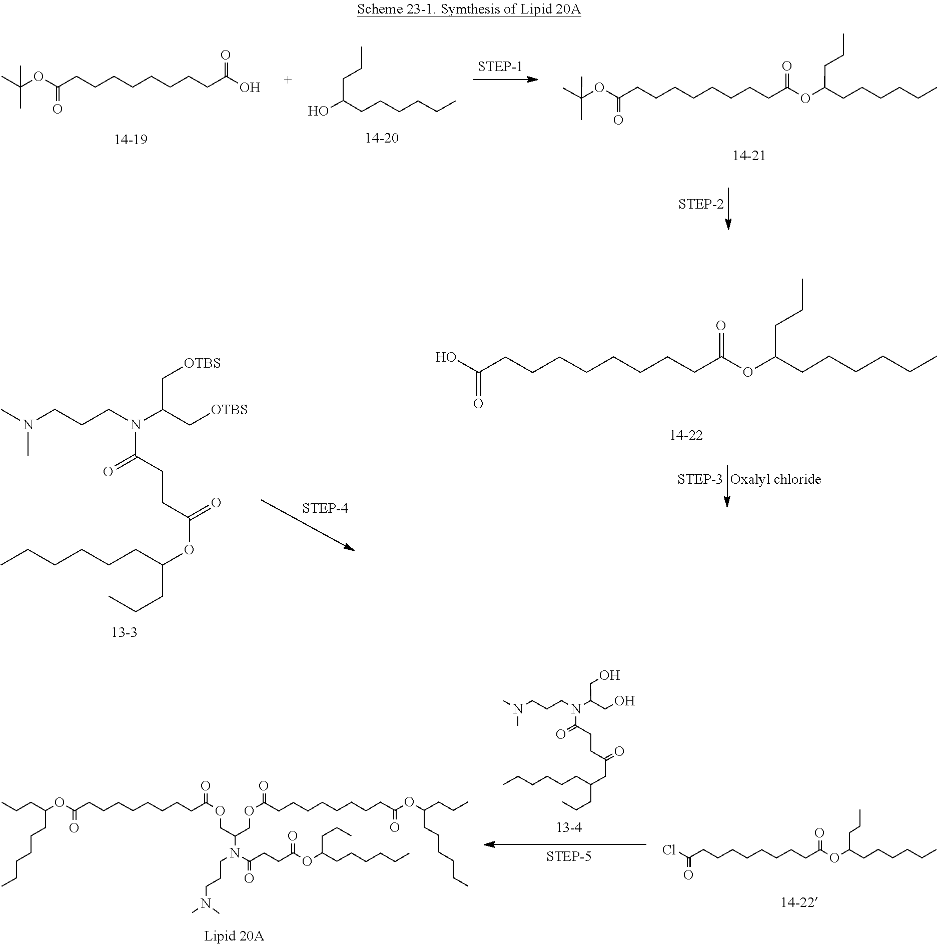

Protected intermediate 13-3 (400 mg, 0.62 mmol, 1 eq.) was treated with Hydrogen Fluoride/Pyridine (15.5 mmol, 1.11 mL, 25.0 eq.) in 6.0 mL THF at room temperature for 2 hours and deprotection was confirmed by TLC and mass spectrometry. Acid intermediate 14-22 (3.72 mmol, 1.27 g, 1 eq.) was converted to the corresponding acid chloride 14-22′ using oxalyl chloride (12.6 mmol, 1.1 mL, 3.4 eq.) and DMF (40 μL, catalytic quantity) in 5.0 mL of Toluene at room temperature for 2 hours and conversion to chloride intermediate by TLC.

Crude di-hydroxy intermediate 13-4 (258 mg, 0.62 mmol, 1 eq.) and crude acid chloride 14-22′ (3.72 mmol, 1.34 g, 6.0 eq.) were combined with TEA (6.2 mmol, 0.87 mL, 10.0 eq) in 5 mL of toluene at room for 2 hours. Crude product was purified on ISCO column chromatography on silica column eluting with DCM and 10% MeOH in DCM yielding 300 mg of 96% pure (HPLC-CAD) Lipid 20A (see FIG. 4AJ-1 and FIG. 4AJ-2 for characterization by proton NMR, mass spectrometry and LC-CAD purity).

### Synthesis of Lipid 21 and its Isomer

An isomer of lipid 21 was synthesized as provided in scheme 24-1 below. Briefly, alcohol 13-78 was accessed by nucleophilic addition to aldehyde 13-77 using diethyl zinc (Step 1) and subsequently used in the ring opening addition to cyclic anhydride 13-52 to access intermediate 13-79. O-acylation of dihydroxyacetone with intermediate 13-79 using conditions described in Lipid 17 synthesis yielded ketone 13-80. Reductive amination of 13-80 with amine 15-3 using conditions described in the Lipid 17 synthesis yielded intermediate 13-81. Subsequent N-acylation of intermediate 13-81 with acid 13-31 using conditions analogous to those used for Lipid 9 synthesis afforded Lipid 21.

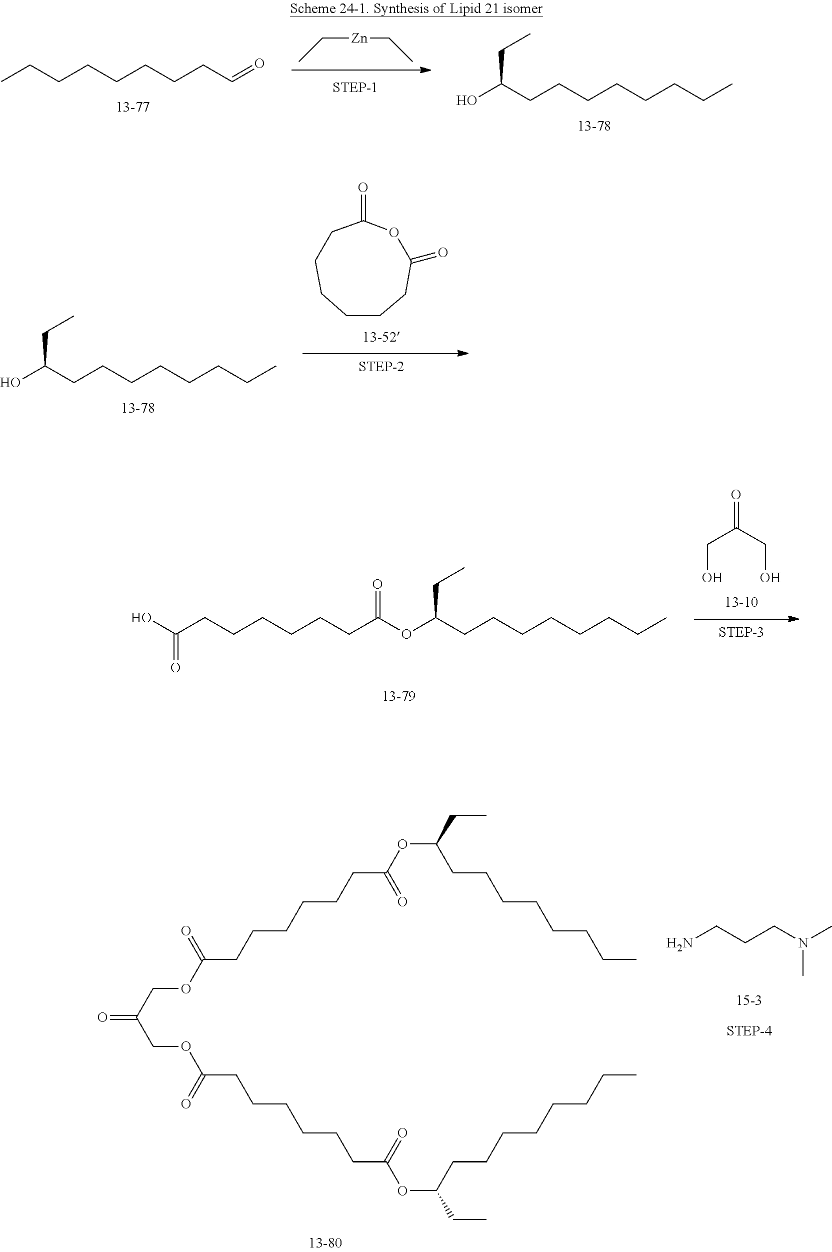

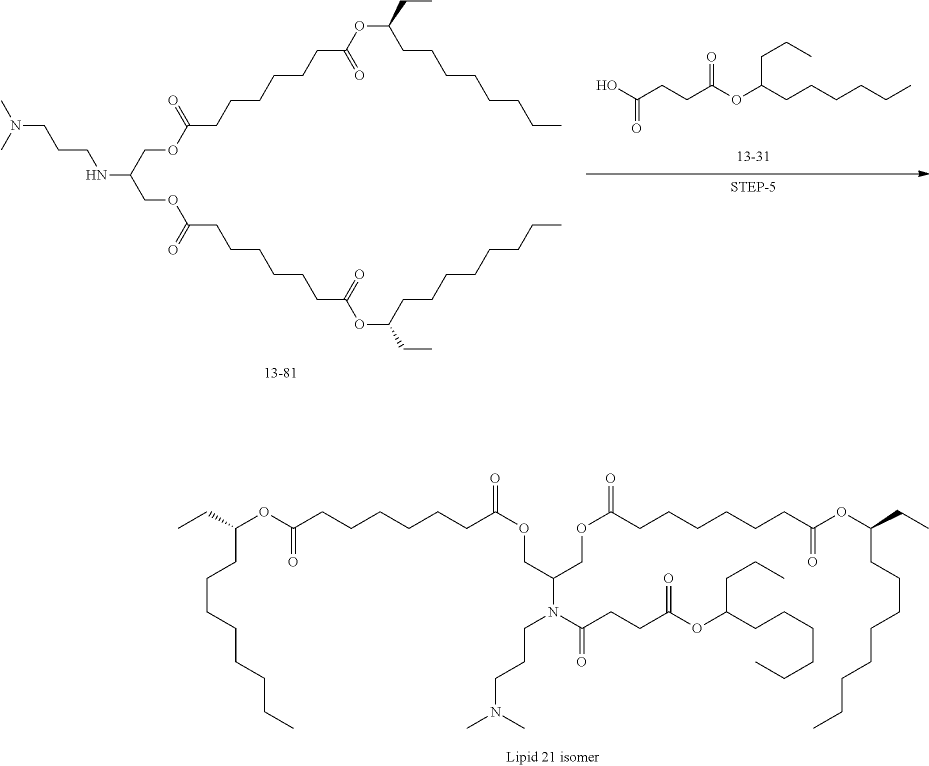

Lipid 21 as a racemic mixture was synthesized as provided in scheme 24-2 below. Briefly, Lipid 21 (racemate) was accessed using methods analogous to those described for Lipid 21 isomer except using ethyl lithium for accessing the racemic alcohol in Step 1.

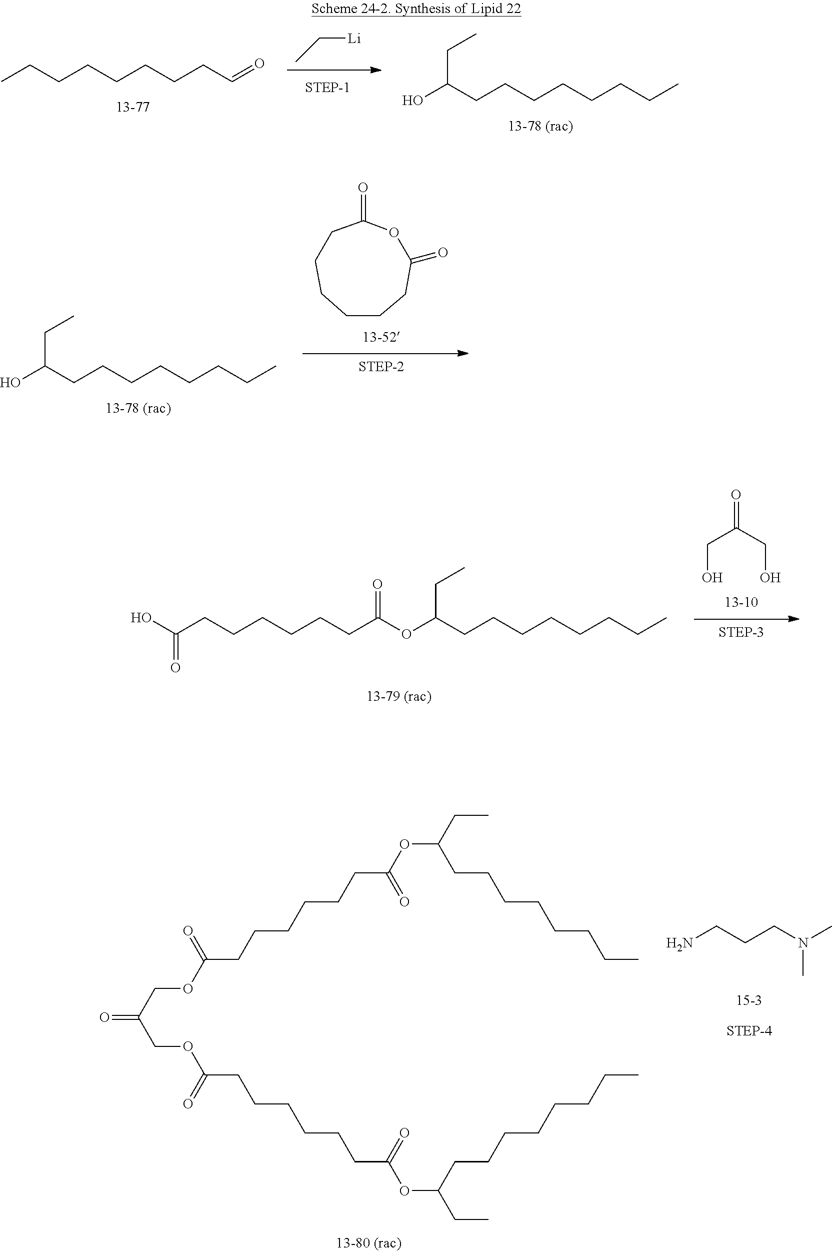

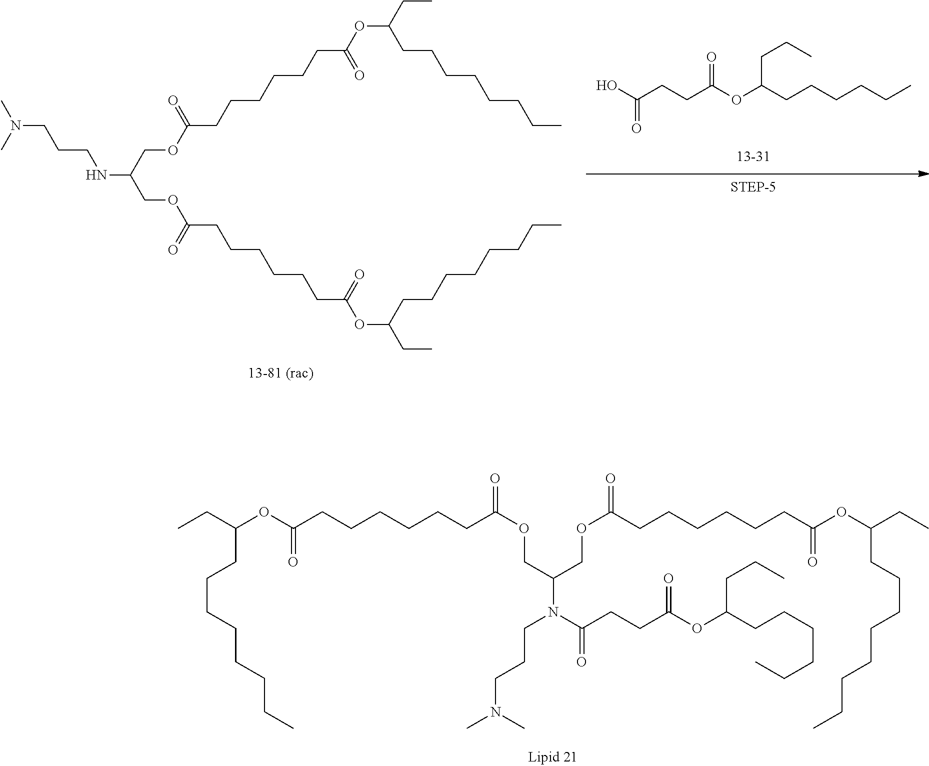

### Synthesis of Lipid 21A

Starting material 3-hydroxyoctanol, 13-66 (12.4 mmol, 1.61 g, 1 eq.) was acylated with sebacic acid 13-65 (24.8 mmol, 5.0 g, 2.0 eq.) using EDCI (14.9 mmol, 2.83 g, 1.2 eq.), DMAP (1.5 mmol, 183 mg, 0.12 eq.) and TEA (62.0 mmol, 8.6 mL, 5.0 eq.) in a mixture of 25 mL of DCM and 25 mL DMF at room temperature, RT, overnight to obtain 2.2 g (56%) of acid intermediate, 13-67. Starting material, 1,3-dihydroxyacetone, 13-10 (3.2 mmol, 286 mg, 1.0 eq.) was diacylated with acid Intermediate 13-67 (7.0 mmol, 2.2 g, 2.2 eq.) using EDCI (7.0 mmol, 1.33 g, 2.2 eq.), DIPEA (7.0 mmol, 1.22 mL, 2.2 eq.) and DMAP (1.6 mmol, 197 mg, 0.5 eq.) in 10 mL of DCM at room temperature overnight to obtain 1.4 g (65%) of ketone intermediate 13-68.

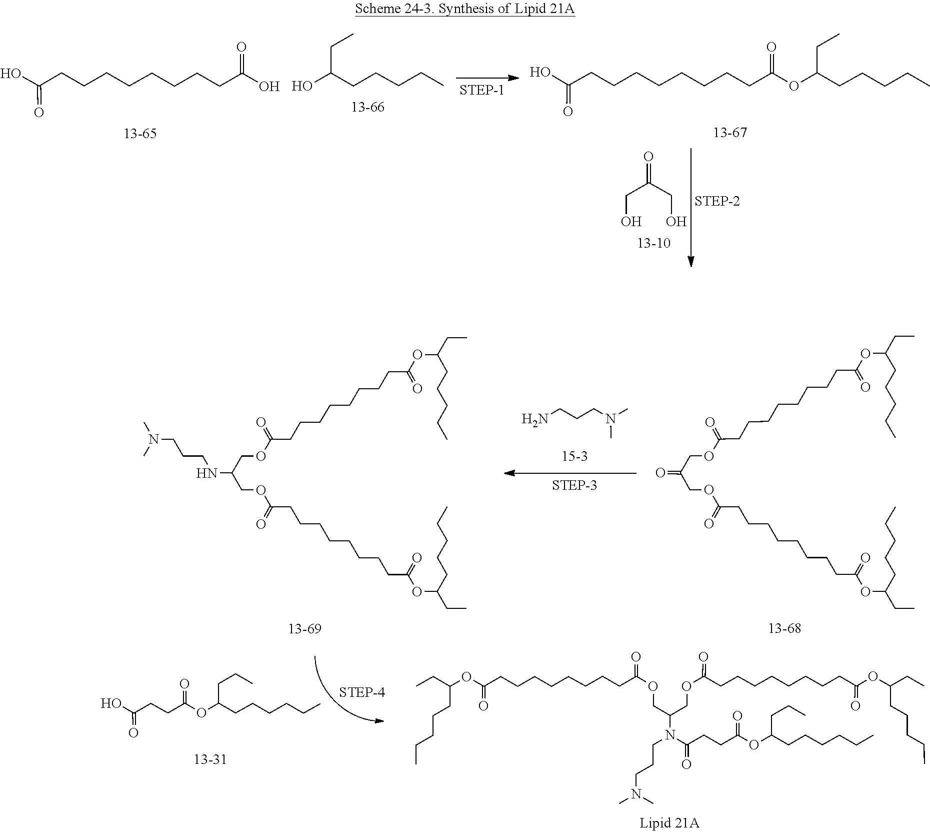

Intermediate 13-68 (2.05 mmol, 1.4 g, 1.0 eq.) was converted to diamine intermediate 13-69 by reductive amination with N,N-dimethylamino-3-aminopropane, 15-3 (514 μL, 2.0 eq, 4.10 mmol) using Na(OAc)₃BH (10 mg, 1.2 eq, 2.46 mmol)), acetic acid (236 μL, 2.0 eq, 4.10 mmol) in 5 mL of DCM at room temperature for 3 hours. Purification of crude product by silica column chromatography (10% MeOH in DCM, 1% NH₄OH) yielded 480 mg (32%) of purified Intermediate 13-69. Acid Intermediate 13-31 (1.86 mmol, 484 mg, 1 eq.) was converted to the corresponding acid chloride 13-31′ using Oxalyl chloride (540 μL, 3.4 eq, 6.38 mmol) and DMF (20 μL, catalytic quantity) in 3 mL of Toluene at room temperature for 2 hours and crude acid chloride 13-31′, (518 mg, 3.0 eq, 1.88 mmol), TEA, Toluene (3.0 mL), RT, Overnight was used for N-acylation of diamine intermediate 13-69 (0.3 mmol, 0.2 g, 1 eq.) using TEA (435 μL, 5.0 eq, 3.13 mmol) and in 3 mL of Toluene at room temperature overnight to yield Lipid 21A. Crude product was purified 2× by column chromatography (Hexanes and EtAc, then DCM: 10% MeOH in DCM on silica) to obtain 51 mg of pure Lipid 21A (>99% by HPLC-UV and 95% by HPLC-CAD) and characterized by proton NMR and mass spectrometry (FIG. 4Y-1 and FIG. 4Y-2 for proton NMR and HPLC-CAD and Table 5 for Mass Spectrometry data).

### Synthesis of Lipid 22 and its Isomer

An isomer of lipid 22 was synthesized as provided in scheme 25-1 below. Briefly, alcohol 13-78 (accessed as described for Lipid 21 synthesis above) was used in the ring opening addition to cyclic anhydride 13-73′ to access intermediate 13-82. O-acylation of dihydroxyacetone with intermediate 13-82 using conditions described in Lipid 17 synthesis yielded ketone 13-83. Reductive amination of 13-83 with amine 15-3 using conditions described in the Lipid 17 synthesis yielded intermediate 13-84. Subsequent N-acylation of intermediate 13-84 with acid 13-31 using conditions analogous to those used for Lipid 9 synthesis afforded Lipid 22 isomer.

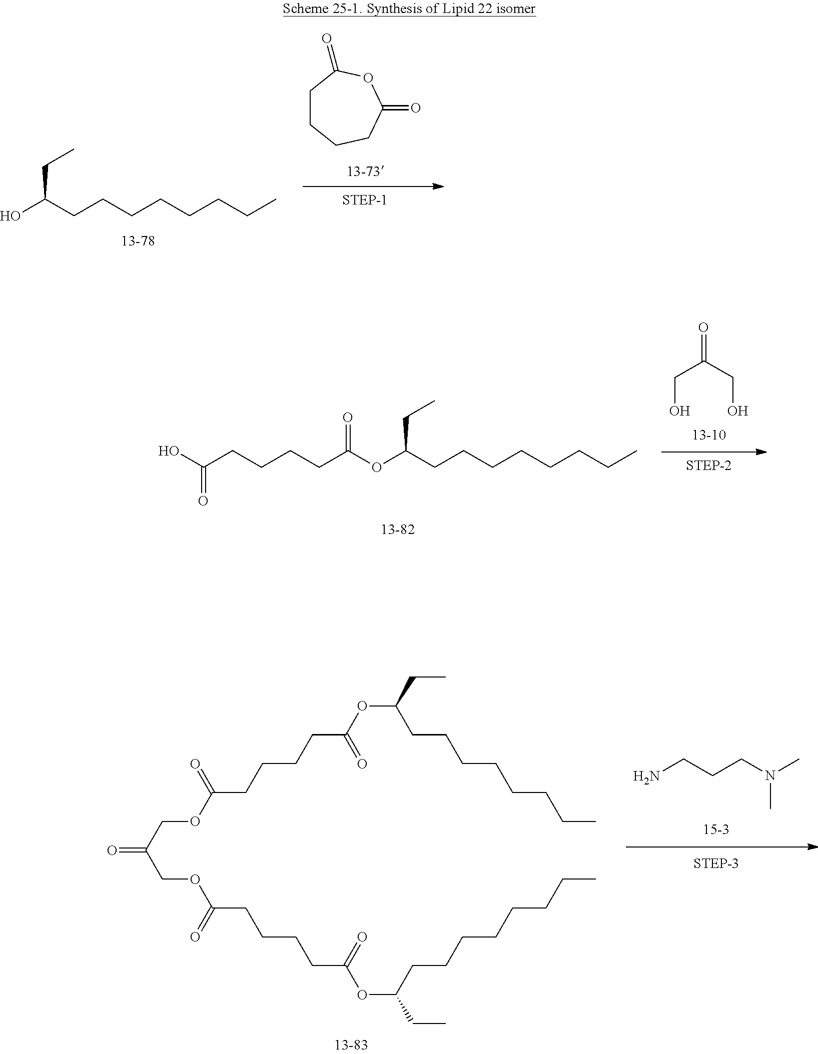

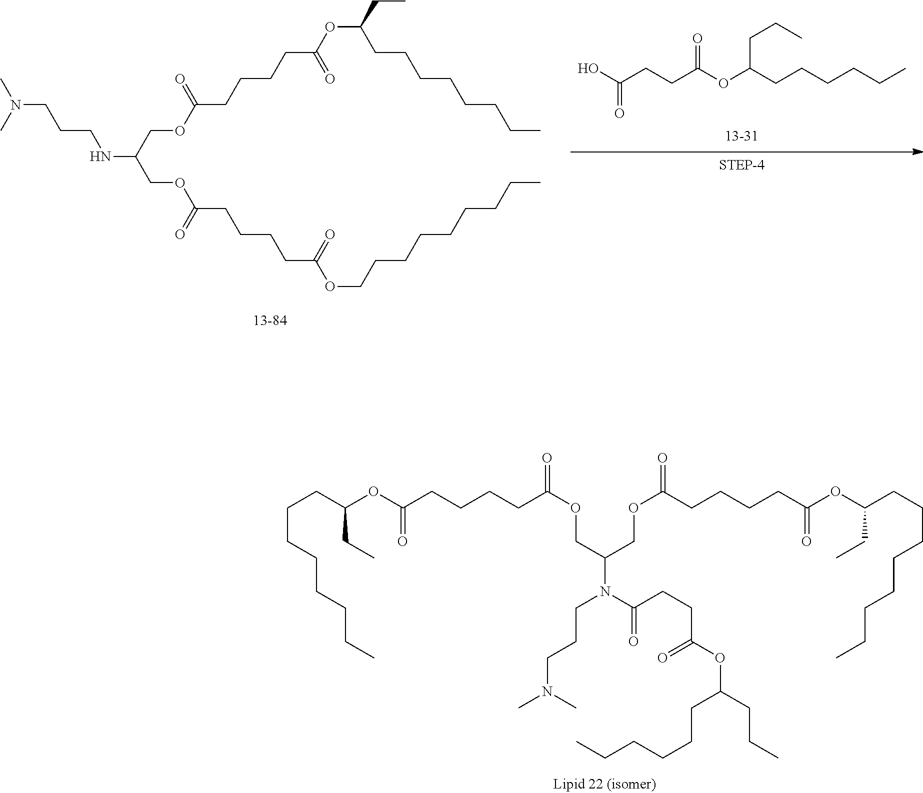 [SMILES: CCCCCCCCCOC(=O)CCCCC(=O)OCC(COC(=O)CCCCC(=O)O[C@@H](CC)CCCCCCCC)NCCCN(C)C ; CCCCCCC(CCC)OC(=O)CCC(=O)O ; CCCCCCCC[C@H](CC)OC(=O)CCCCC(=O)OCC(COC(=O)CCCCC(=O)O[C@@H](CC)CCCCCCCC)N(CCCN(C)C)C(=O)CCC(=O)OC(CCC)CCCCCC]

Lipid 22 as a racemic mixture was synthesized as provided in scheme 25-2 below. Lipid 22 was accessed using methods described for Lipid 22 isomer above by replacing alcohol isomer 13-78 with racemic alcohol 13-78 rac.

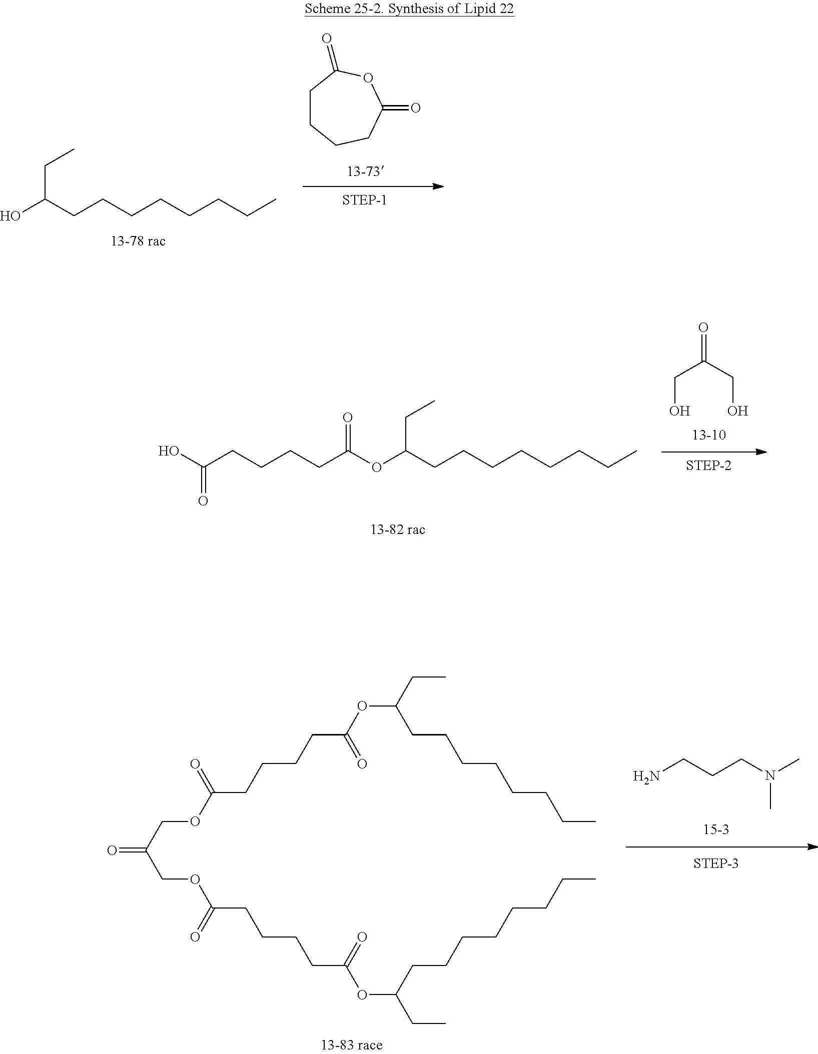

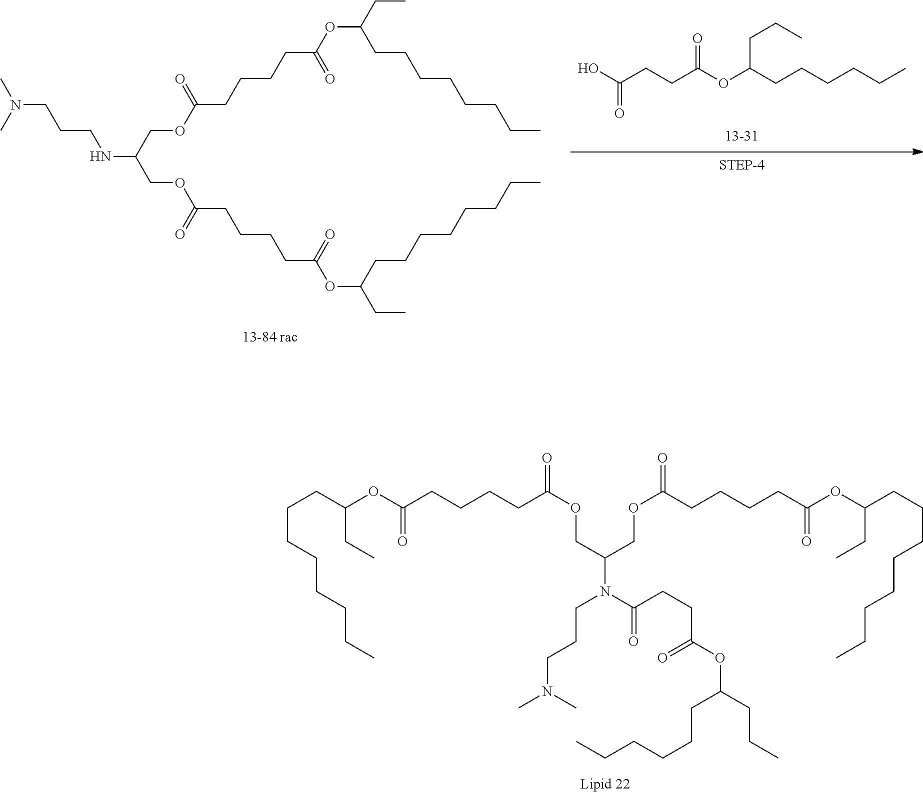 [SMILES: CCCCCCCCC(CC)OC(=O)CCCCC(=O)OCC(COC(=O)CCCCC(=O)OC(CC)CCCCCCCC)NCCCN(C)C ; CCCCCCC(CCC)OC(=O)CCC(=O)O ; CCCCCCCCC(CC)OC(=O)CCCCC(=O)OCC(COC(=O)CCCCC(=O)OC(CC)CCCCCCCC)N(CCCN(C)C)C(=O)CCC(=O)OC(CCC)CCCCCC]

Alternatively, starting material 3-undecanol, 13-78-racemic (31.6 mmol, 5 g, 1 eq.) was acylated with adipic acid, 13-82 (9.8 mmol g, 1.68 g, 2.0 eq.) using EDCI (2.26 g, 1.2 eq, 11.8 mmol), DMAP (143 mg, 0.12 eq, 1.2 mmol) and TEA (6.8 mL, 5.0 eq, 49.0 mmol) in a mixture of 20 mL of DCM and 20 mL DMF at room temperature, overnight to obtain 1.35 g (46%) of acid intermediate, 13-83 rac. Starting material, 1,3-dihydroxyacetone, 13-10 (2.05 mmol, 184 mg, 1.0 eq.) was diacylated with acid Intermediate 13-83-rac (1.35 g, 2.2 eq, 4.5 mmol) using EDCI (856 mg, 2.2 eq, 4.5 mmol), DIPEA (782 μL, 2.2 eq, 4.5 mmol), and DMAP (127 mg, 0.5 eq, 1.03 mmol) in 6 mL of DCM at room temperature overnight to obtain 610 mg (46%) of ketone intermediate 13-84-rac.

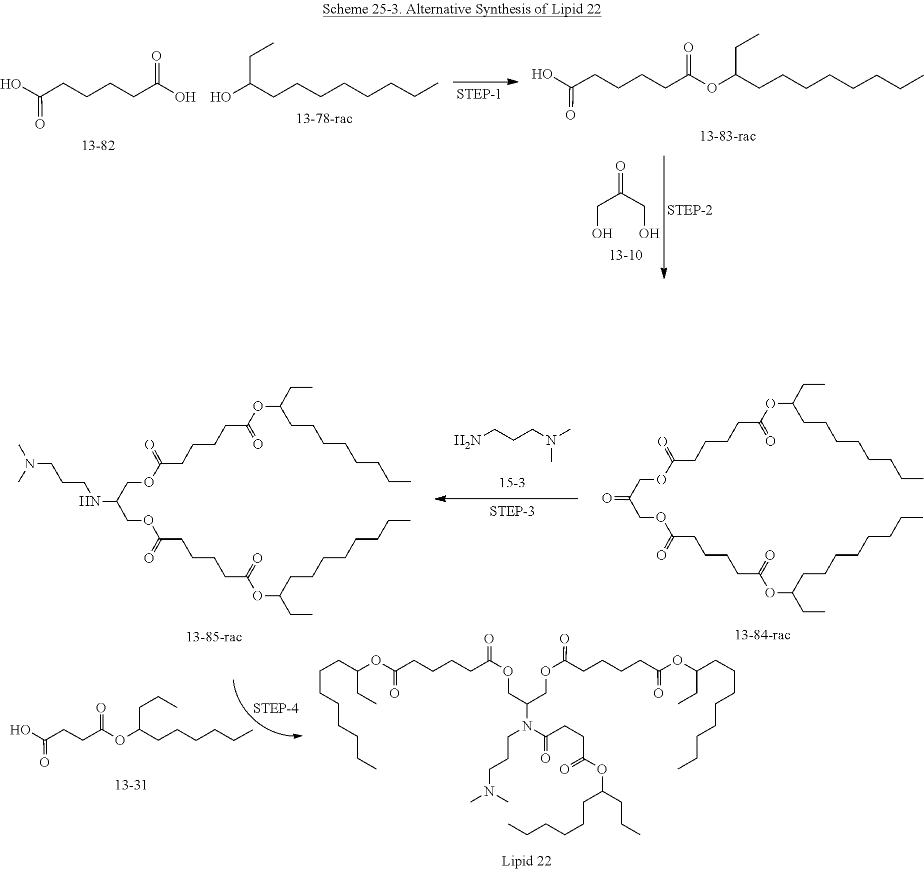

Intermediate 13-84-rac (0.93 mmol, 610 mg, 1.0 eq.) was converted to diamine intermediate 13-85-rac by reductive amination with N,N-dimethylamino-3-aminopropane, 15-3 (1.86 mmol, 233 μL, 2.0 eq.) using Na(OAc)₃BH (1.2 mmol, 249 mg, 1.2 eq.), acetic acid (1.86 mmol, 107 μL, 2.0 eq.) in 3 mL of DCM at room temperature for 3 hours. Purification of crude product by silica column chromatography (10% MeOH in DCM) yielded 210 mg of purified Intermediate 13-85-rac. Acid chloride 13-31′ (0.85 mmol, 233 mg, 3.0 eq.) from a previous batch was used for N-acylation of diamine intermediate 13-85-rac (0.28 mmol, 210 mg, 1 eq.) using TEA (1.4 mmol, 197 μL, 5.0 eq.) in 3 mL of Toluene at room temperature overnight to yield Lipid 22. Crude product was purified 2× by column chromatography (DCM: 10% MeOH in DCM) to obtain 56 mg (60%) of pure Lipid 22 (>99% by HPLC-CAD) and characterized by proton NMR and mass spectrometry (FIG. 4Z-1 and FIG. 4Z-2 for proton NMR and HPLC-CAD and Table 5 for Mass Spectrometry data A,B,C).

### Synthesis of Lipid 23

Lipid 23 was synthesized as provided in scheme 26 below. Briefly, O-acylation of dihydroxyacetone with acid 13-31 using conditions described in Lipid 9 synthesis yielded ketone 13-70. Reductive amination of 13-70 with amine 15-3 using conditions described in the Lipid 9 synthesis yielded intermediate 13-71. Subsequent N-acylation of intermediate 13-71 with acid 13-31 using conditions analogous to those used for Lipid 9 synthesis afforded Lipid 23.

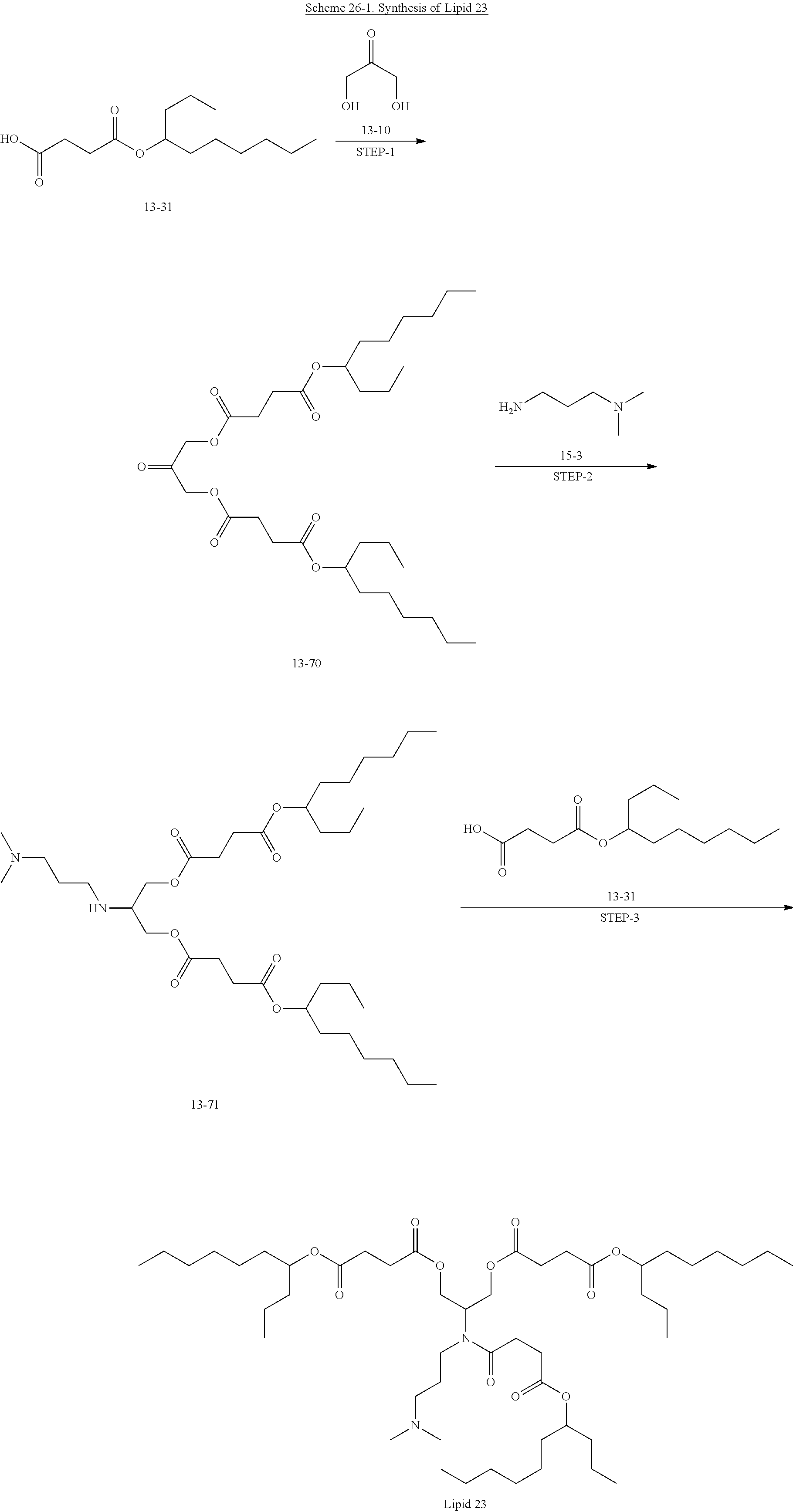

### Synthesis of Lipid 23A

Starting material 3-undecanol, 13-78-racemic (14.4 mmol, 2.47 g, 1 eq.) was acylated with suberic acid, 13-77 (5.0 g, 2.0 eq, 82.7 mmol) using EDCI (3.3 g, 1.2 eq, 17.3 mmol), DMAP (211 mg, 0.12 eq, 1.73 mmol) and TEA (10.0 mL, 5.0 eq, 72.0 mmol) in a mixture of 20 mL of DCM and 20 mL DMF at room temperature overnight to obtain 2 g (43%) of acid intermediate, 13-879-rac. Starting material, 1,3-dihydroxyacetone, 13-10 (2.8 mmol, 250 mg, 1.0 eq.) was diacylated with acid Intermediate 13-79-rac (2.0 g, 2.2 eq, 6.1 mmol) using EDCI (1.16 g, 2.2 eq, 6.1 mmol), DIPEA (1.06 mL, 2.2 eq, 6.1 mmol), and DMAP (172 mg, 0.5 eq, 1.4 mmol) in 8 mL of DCM at room temperature overnight to obtain 690 mg (35%) of ketone intermediate 13-80-rac.

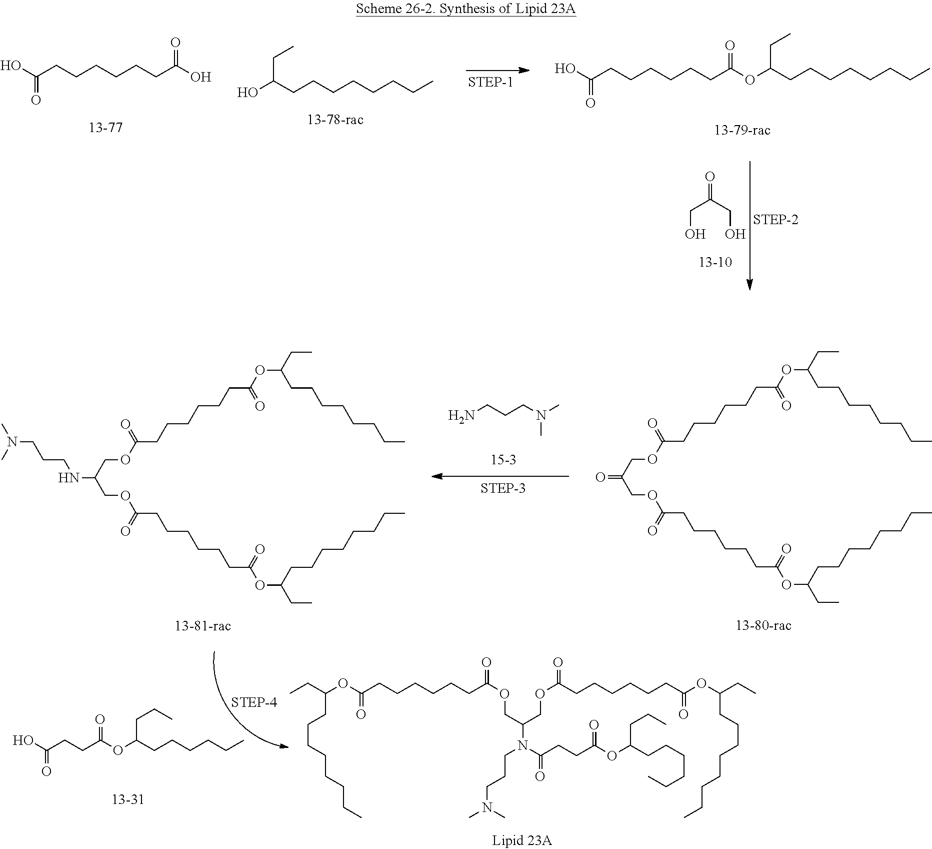

Intermediate 13-80-rac (0.97 mmol, 690 mg, 1.0 eq.) was converted to diamine intermediate 13-81-rac by reductive amination with N,N-dimethylamino-3-aminopropane, 15-3 (1.94 mmol, 243 μL, 2.0 eq.) using Na(OAc)₃BH (1.2 mmol, 249 mg, 1.2 eq.), acetic acid (1.94 mmol, 112 μL, 2.0 eq.) in 3 mL of DCM at room temperature for 3 hours. Purification of crude product by silica column chromatography (10% MeOH in DCM) yielded 520 mg of purified Intermediate 13-81-rac. Acid chloride 13-31′ (1.63 mmol, 447 mg, 2.5 eq.) from a previous batch was used for N-acylation of diamine intermediate 13-81-rac (0.65 mmol, 520 mg, 1 eq.) using TEA (3.25 mmol, 458 μL, 5.0 eq.) in 4 mL of toluene at room temperature overnight to yield Lipid 23A. Crude product was purified 2× by column chromatography (DCM: 10% MeOH in DCM) to obtain 230 mg (31%) of pure Lipid 23A (>99% by HPLC-UV and 96% by HPLC-CAD) and characterized by proton NMR and mass spectrometry (FIG. 4AA-1 and FIG. 4AA-2 for proton NMR and HPLC-CAD and Table 5 for Mass Spectrometry data).

### Synthesis of Lipid 24

Lipid 24 was synthesized as provided in scheme 27 below. Briefly, acid 13-34 was accessed by O-acylation of mono-protected di-acid 13-72 with alcohol 13-29 and subsequent deprotection of intermediate 13-73 to yield acid, 13-74. O-acylation of dihydroxyacetone with intermediate 13-74 using conditions described in Lipid 17 synthesis yielded ketone 13-75. Reductive amination of 13-75 with amine 15-3 using conditions described in the Lipid 17 synthesis yielded intermediate 13-76. Subsequent N-acylation of intermediate 13-76 with acid 13-31 using conditions analogous to those used for Lipid 9 synthesis afforded Lipid 24.

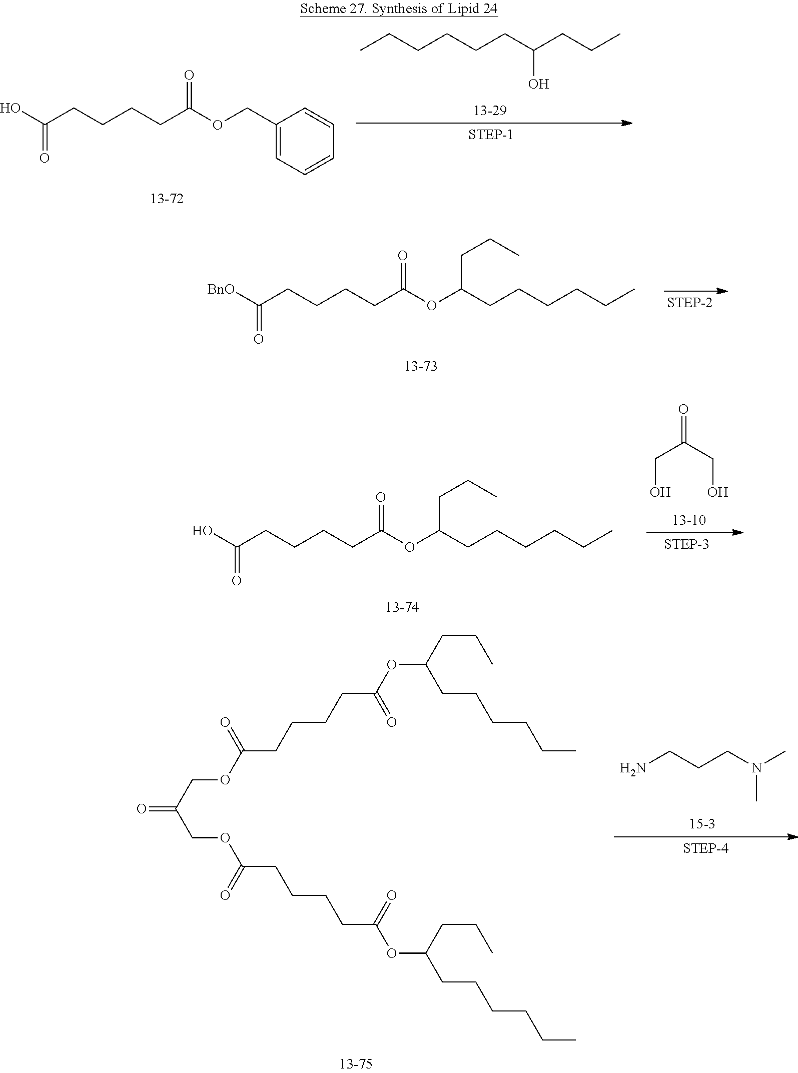

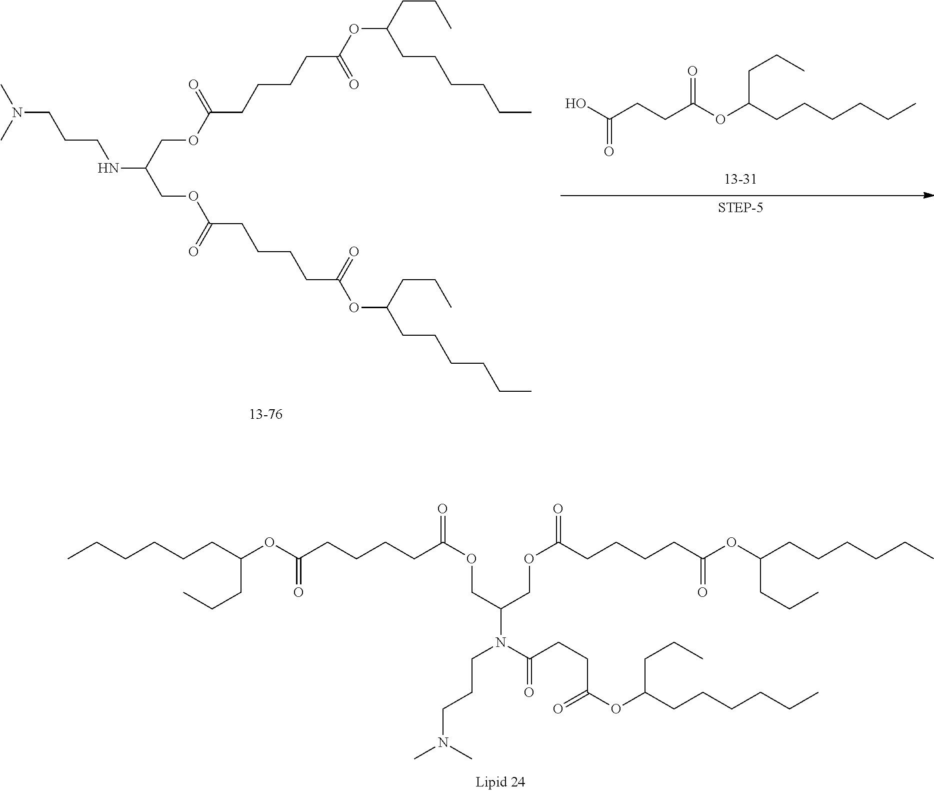 [SMILES: CCCCCCC(CCC)OC(=O)CCCCC(=O)OCC(COC(=O)CCCCC(=O)OC(CCC)CCCCCC)NCCCN(C)C ; CCCCCCC(CCC)OC(=O)CCC(=O)O ; CCCCCCC(CCC)OC(=O)CCCCC(=O)OCC(COC(=O)CCCCC(=O)OC(CCC)CCCCCC)N(CCCN(C)C)C(=O)CCC(=O)OC(CCC)CCCCCC]

### Synthesis of Lipid 25

Lipid 25 was synthesized as provided in scheme 28 below. Briefly, ring opening addition of alcohol 13-29 to cyclic anhydride 13-52′ yielded acid intermediate 13-85. O-acylation of dihydroxyacetone with intermediate 13-85 using conditions described in Lipid 17 synthesis yielded ketone 13-86. Reductive amination of 13-86 with amine 15-3 using conditions described in the Lipid 17 synthesis yielded intermediate 13-87. Subsequent N-acylation of intermediate 13-87 with acid 13-31 using conditions analogous to those used for Lipid 9 synthesis afforded Lipid 25.

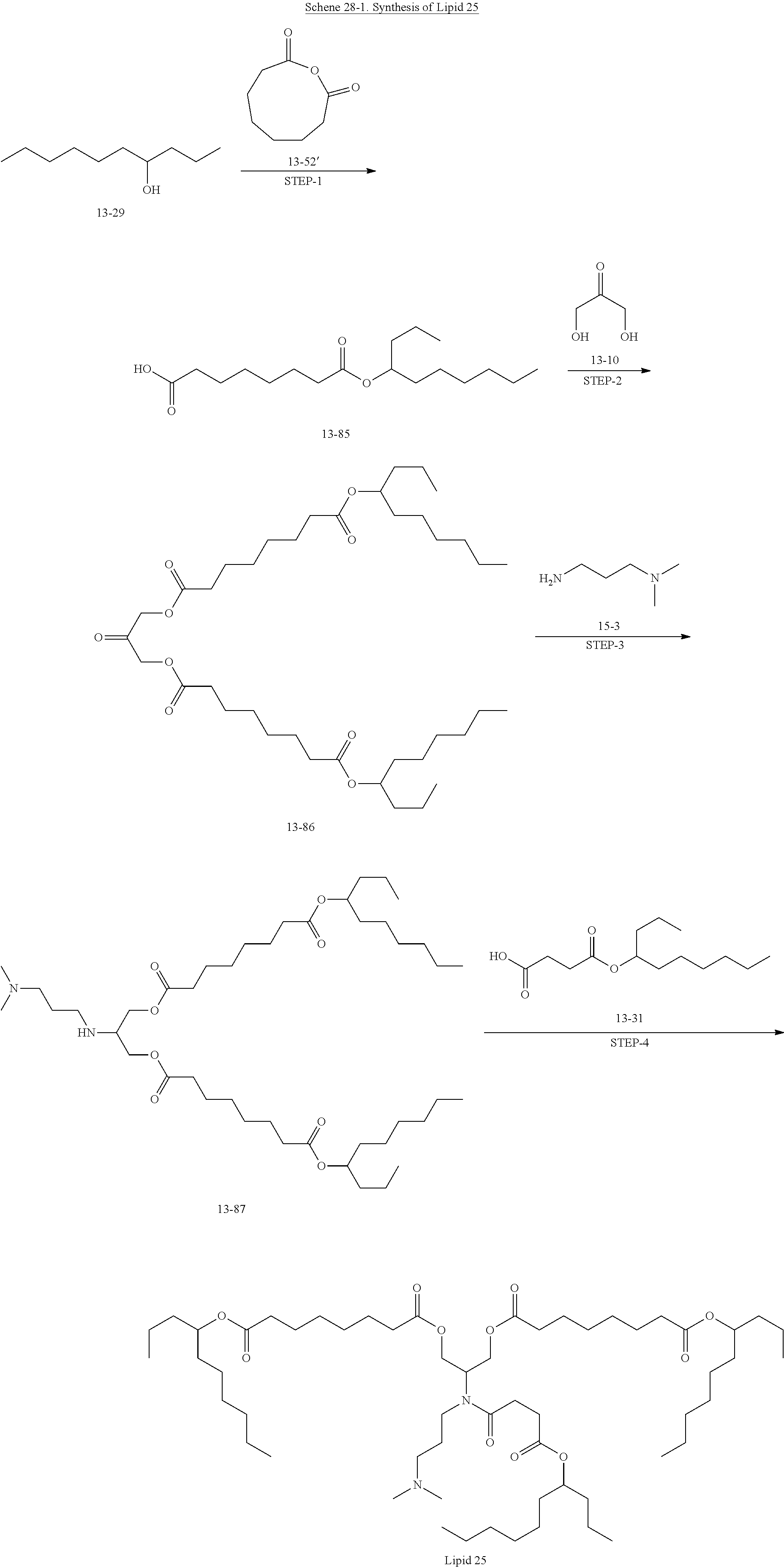

### Synthesis of Lipid 25A

Starting material benzyl protected glycolic acid 14-24 (15.4 mmol, 2.61 g, 1 eq.) was acylated with 2-hexyldecanoic acid 14-25 (6.10 g, 96%, 1.48 eq, 22.84 mmol) using EDCI (5.80 g, 1.97 eq, 30.26 mmol), DMAP (0.40 g, 0.21 eq, 3.27 mmol) and DIPEA (5.4 mL g, 1.97 eq, 30.33 mmol) in 60 mL of dichloromethane at room temperature overnight yielding 2.75 g (49%) of protected intermediate ester 14-26. Intermediate 14-26 (10.3 mmol, 4.1 g, 1.0 eq.) was deprotected using Pd/C (930 mg, 32% w/w), EtOAc (35 mL) at room temperature overnight yielding 1.72 g (82%) of acid intermediate 14-27.

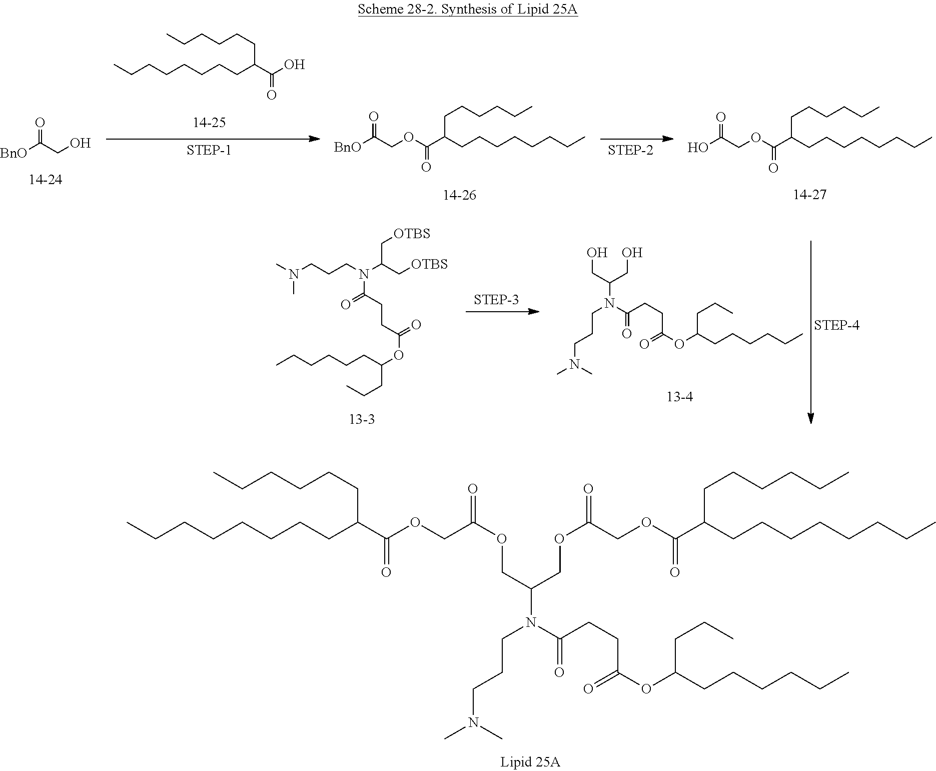

Protected intermediate 13-3 (600 mg, 0.9 mmol, 1 eq.) was treated with Hydrogen Flouride/Pyridine (0.92 g, 10.0 eq, 9.3 mmol) in 4.0 mL THF at room temperature for 2 hours yielding dihydroxyl intermediate 13-4. Deprotection was confirmed by TLC and mass spectrometry. Acid intermediate 14-27 (5.4 mmol, 1.72 g, 1 eq.) was converted to the corresponding acid chloride 14-27′ using oxalyl chloride (2.36 g, 3.4 eq, 18.59 mmol) and DMF (100 μL, catalytic quantity) in 5.0 mL of Toluene at room temperature for 2 hours and conversion to chloride intermediate was confirmed by TLC.

Crude di-hydroxy intermediate 13-4 (350 mg, 0.84 mmol, 1 eq.) and crude acid chloride 14-27′ (1.58 g, 6.0 eq, 5.04 mmol) were combined with TEA (0.85 g, 10.0 eq, 8.4 mmol) in 9 mL of toluene at room temperature, overnight. Crude product was purified on ISCO column chromatography on silica column eluting with DCM and 10% MeOH in DCM yielding 240 mg (85%) of 99% pure (HPLC-CAD) Lipid 25A. (see FIG. 4AB-1 and FIG. 4AB-2 for characterization by proton NMR and LC-CAD purity and Table 5 for Mass Spectrometry data).

### Synthesis of Lipid 27

Lipid 27 was synthesized as provided in scheme 29 below and as follows. Ring opening of starting material caprolactone 14-30 (2 g, 17.5 mmol) using 5 mL of 0.5 M NaOH at room temperature for 2 hours yielded 6-hydroxyhexanoic acid 14-31 (1.8 g, 78%). Additional 14-31 was produced in a second 2 g scale caprolactone hydrolysis reaction using identical conditions to obtain 1.9 g (82%) of 14-31. 6-hydroxyhexanoic acid 14-31 (1 g, 7.5 mmol) was benzyl protected using DBU (1.38 g, 1.2 eq, 9 mmol), Benzylbromide (1.68 g, 1.3 eq, 9.8 mmol) in 6 mL of MeOH and 10 mL of DMF at 0° C. to room temperature, overnight yielding protected intermediate 14-32 (1.3 g, 77%). Additional 14-31 (2 g, 15.1 mmol) was protected using DBU (2.76 g, 1.2 eq, 18.1 mmol), Benzylbromide (3.36 g, 1.3 eq, 19.6 mmol) in 12 mL of MeOH and 20 mL of DMF at 0° C. to room temperature, overnight yielding protected intermediate 14-32 (2.65 g, 79%).

Intermediate 14-32 (2.45 g, 1 eq, 11 mmol) was used to acylate acid 14-25 (2.59 g, 1.5 eq, 10 mmol) using EDCI (2.58 g, 2 eq, 13.4 mmol), DIPEA (1.74 g, 2 eq, 13.4 mmol) and DMAP (0.16 g, 0.2 eq, 1.3 mmol) in 20.0 mL DCM at room temperature overnight. Crude product was purified by column chromatography (1×) yielding 4.8 g (94%) of pure protected intermediate 14-33. Subsequent deprotection of 14-33 (4.8 g, 22 mmol) by hydrogenation on Pd/C/Hydrogen gas (0.6 g, 20% w/w) in 30 mL of ethylacetate yielded 3.68 g (95%) of free acid 14-34 of column purified material.

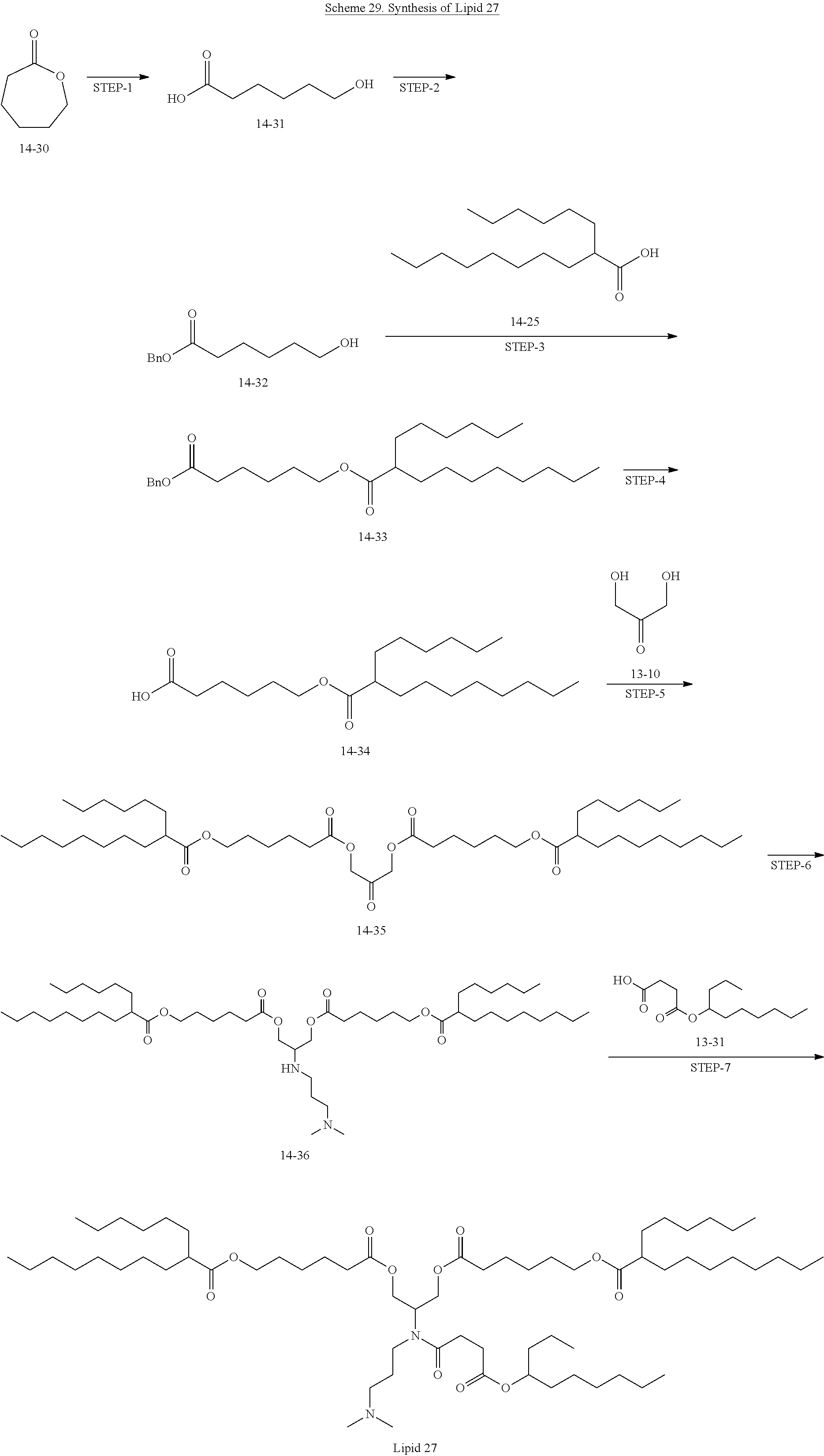

Acid intermediate 14-34 (1.74 g, 2.5 eq, 5.5 mmol) was used to acylate dihydroxyacetone, 13-10 (200 mg, 1.0 eq, 6 mmol), EDCI (1.06 g, 2.5 eq, 5.5 mmol), DIPEA (0.71 g, 2.5 eq, 5.5 mmol), and DMAP (54 mg, 0.2 eq, 0.4 mmol) in 10.0 mL DCM at room temperature, overnight to obtain ketone 14-35. Crude product was purified by column chromatography to obtain 1.27 mg (76%) of pure 14-35. Reductive amination of 14-35 (1.26 mg, 1.0 eq, 1.5 mmol) with amine 15-3 (0.32 g, 2.0 eq, 3.1 mmol) using acetic acid (0.19 g, 2.0 eq, 3.1 mmol) and Na(OAc)₃BH (0.50 g, 1.5 eq, 2.3 mmol) in 15 mL DCM at room temperature of 3 hours yielded intermediate 14-36 (330 mg, 34%).

Compound 13-31 (290 mg, 1.1 mmol) from a previous batch was converted to the corresponding acid chloride 13-31′ using oxalyl chloride (0.48 g, 3.4 eq, 3.8 mmol) and DMF (20 μL, catalytic quantity) in 4 mL of Toluene for 2 hours at room temperature. Crude 13-31′ (0.29 g, 3.0 eq, 1.1 mmol) was used for N-acylation of 14-36 (330 mg, 1.0 eq, 0.3 mmol) using TEA (0.26 mL, 5.0 eq, 1.8 mmol) 5 mL toluene at room temperature, overnight to obtain column purified Lipid 20 (180 mg, 43%) of >98% purity (HPLC-CAD).

See FIG. 4AE-1 and FIG. 4AE-2 for Lipid 27 NMR spectrum, and reverse phase LC-CAD chromatogram, and Table 5 for product mass.

### Synthesis of Lipid 28

Lipid 28 was synthesized as provided in scheme 30 below and as follows. Intermediate 14-36 was produced as described above (see Synthesis of Lipid 27).

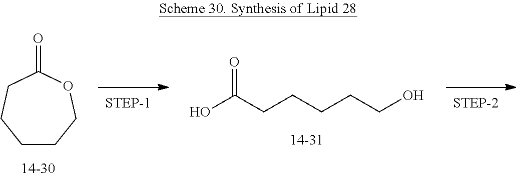 [SMILES: O=C1CCCCCO1 ; O=C(O)CCCCCO]

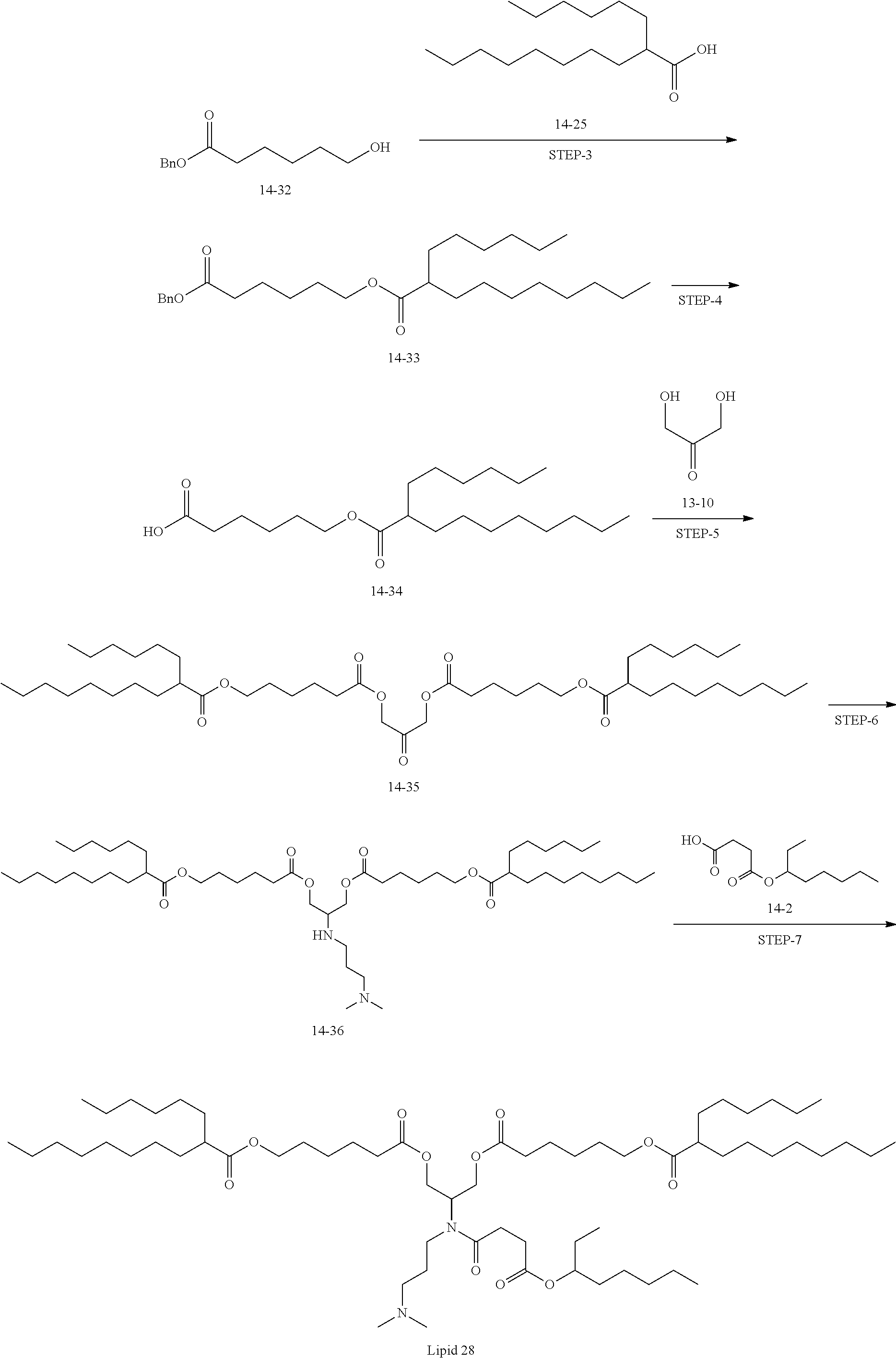

Acid intermediate 14-2 was produced by acylation of starting material 3-hydroxyoctanol 14-1 with succinic acid using 13-30 (1.53 g, 2.0 eq, 15.3 mmol) and DMAP (0.93 g, 1.0 eq, 7.6 mmol) and 2 mL of Pyridine in a mixture of 2 mL of THF and 5 mL of DCM to obtain 1.1 g (64%) of 14-2. 14-2 (348 mg, 1.51 mmol) was converted to the corresponding acid chloride 14-2″ using oxalyl chloride (0.651 g, 5.13 mmol, 3.4 eq) and DMF (40 μL, catalytic quantity) in 3 mL of Toluene for 2 hours at room temperature. Crude 14-2′ (0.348 g, 1.51 mmol, 3.1 eq.) was used for N-acylation of 14-36 (430 mg, 1.51) using TEA (0.41 mL, 2.94 mmol, 6.02 eq) in 6 mL toluene and 2.5 mL DCM at room temperature, overnight to obtain column purified (eluting with 10% methanol in DCM) Lipid 20 (148 mg, 28%) of 85% purity (HPLC-CAD). Second column purification (eluting with 5% methanol in DCM) yielded 115 mg of 94% purity (HPLC-CAD).

See FIG. 4AF-1 for Lipid 27 NMR spectrum, FIG. 4AF-2 for Lipid 28 reverse phase LC-CAD chromatogram, and Table 5 for product mass.

### Synthesis of Lipid 29

Benzyl protected malic acid 14-4 (14.1 mmol, 3.18 g, 1 eq.) was acylated with N-decanoic acid 14-12 (3.86 g, 1.5 eq, 21.2 mmol) using HATU (8.1 g, 1.5 eq, 21.2 mmol), DBU (4.3 g, 2.0 eq, 28.3 mmol) in 30 mL of DMF at room temperature overnight yielding 1.9 g (36%) of protected intermediate ester 14-13. Intermediate 14-13 (5.2 mmol, 1.9 g, 1 eq.) was acylated with hexanoyl chloride 14-6, (2.8 g, 4.0 eq, 20.8 mmol), TEA (2.63 g, 5.0 eq, 26.0 mmol), DMAP (127 mg, 0.2 eq, 1.0 mmol) in 20 mL of Toluene at room temperature, overnight to obtain intermediate intermediate 14-14 (630 mg, 26%). Intermediate 14-14 (2.8 mmol, 1.3 g, 1.0 eq.) was deprotected using Pd/C (260 mg, 32% w/w) in 15 mL Ethyl Acetate at room temperature overnight yielding 1.025 g (98%) of acid intermediate 14-15.

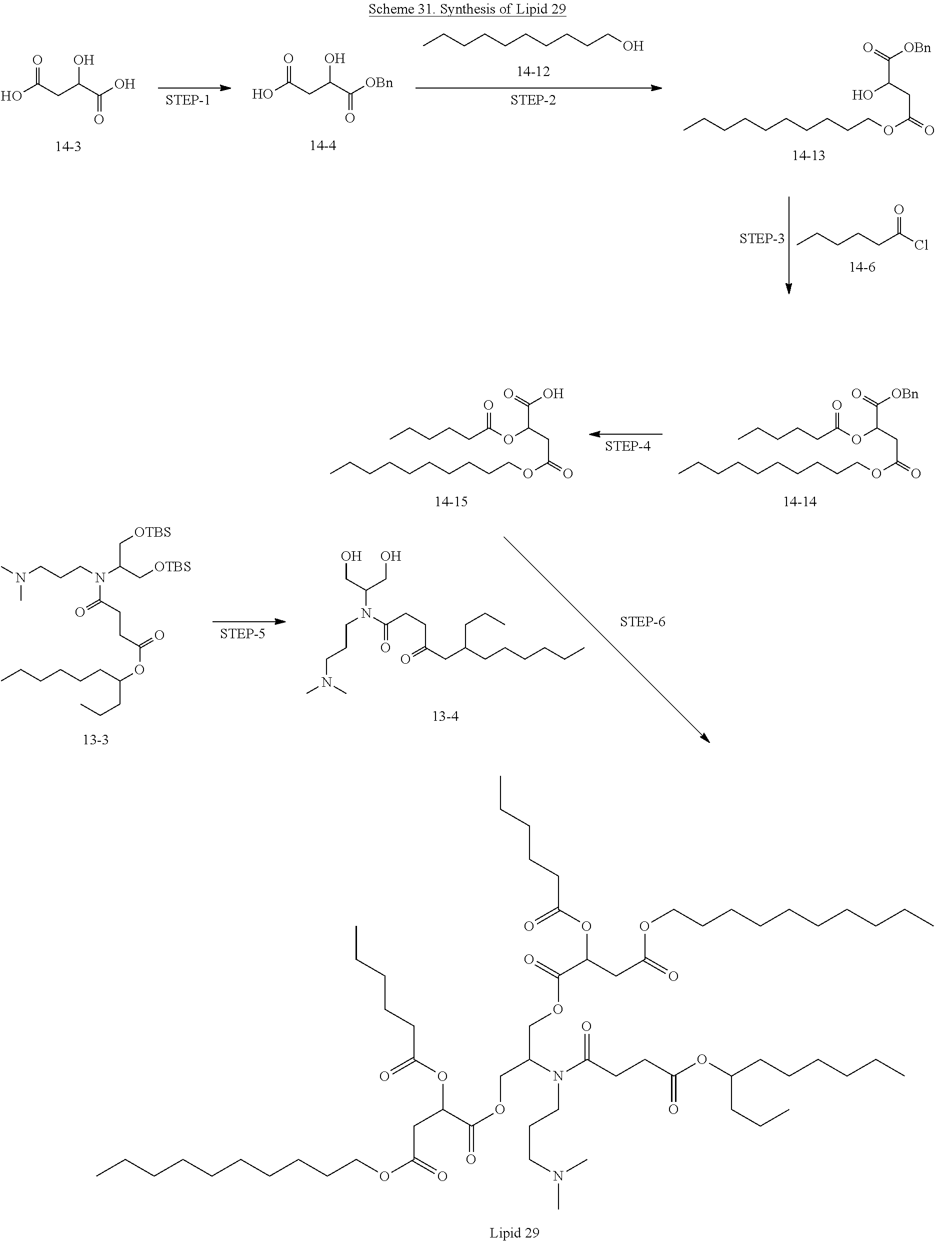

Acid intermediate 14-15 (2.6 mmol, 1.0 g, 1 eq.) was converted to the corresponding acid chloride 14-15′ using oxalyl chloride (0.82 mL, 3.4 eq, 9.1) and DMF (100 μL, catalytic quantity) in 6.0 mL of Toluene at room temperature for 2 hours and conversion to chloride intermediate was confirmed by TLC.

Protected intermediate 13-3 (0.72 mmol, 300 mg) using HF-pyridine, (0.71 mL, 10.0 eq, 7.2 mmol) in 4.0 mL THF at room temperature, overnight and crude di-hydroxy intermediate 13-4 (195 mg, 0.46 mmol, 1 eq.) and crude acid chloride 14-15′ (1.097 g, 6.0 eq, 2.7 mmol) were combined with TEA (0.64 mL, 10.0 eq, 4.6 mmol) in 5 mL of toluene at room temperature, overnight. Crude product was purified on ISCO column chromatography on silica column eluting with DCM and 10% MeOH in DCM yielding to obtain 270 mg (51%) of >99% pure (HPLC-CAD) Lipid 29. (see FIG. 4AG-1 and FIG. 4AG-2 for characterization by proton NMR and LC-CAD purity, and Table 5 for Mass Spectrometry data).

### Synthesis of Lipid 31

Lipid 31 was synthesized as provided in scheme 32 below and as follows. Starting material 15-1 (68 mmol, 10 g, 1 eq.) was treated with p-toluene sulfonyl chloride (70 mmol, 13.3 g, 1.03 eq.) using Pyridine (80 mmol, 10.1 ml, 1.2 eq.) in 150 mL DCM to obtain protected intermediate 15-2. Crude product was recrystallized in ethyl acetate and hexane yielding 20.4 g (99%) of pure intermediate 15.2. Intermediate 15-4 was accessed by reaction of 15-2 (16.5 mmol, 5 g, 1.2 eq.) and diamine 15-3 (33 mmol, 3.35 g, 2 eq.) in 40 mL dioxane under reflux conditions. Crude product was purified by column chromatography to obtain 3.5 g (91%) of pure intermediate 15-4. N-acylation of 15-4 (108 mg, 0.268 mmol) using nonanoic acid 13-12 (0.67 mmol, 106 mg, 2.5 eq) using EDCI (0.67 mmol, 128 mg, 2.5 eq.) and DIEA (0.67 mmol, 86 mg, 2.5 eq) and DMAP (3 mg) in 10 mL DCM yielded amine 15-5. Crude product was purified by column chromatography to obtain 113 mg (65%) of purified diamine 15-5. Diol intermediate 15-6 was accessed by deprotection of 15-5 (113 mg) in 4 mL of 1M HCl and THF (1:3 v/v) at room temperature for 8 hours in quantitative yield (102 mg). Intermediate 15-6 (0.3 mmol, 100 mg, 1 eq) was acylated with linoleic acid 1-5 (0.9 mmol, 250 mg, 3 eq) using EDCI (0.9 mmol, 172 mg, 3 eq), DIPEA (0.9 mmol, 116 mg) and DMAP (10 mg, catalytic quantity) to obtain Lipid 31. Crude product was purified by column chromatography yielding 120 mg (46%) of pure Lipid 31 (>99% purity by LC-ELSD) and characterized by proton NMR and Mass Spectrometry (see FIG. 4R-1 for Lipid 31 NMR spectrum, FIG. 4R-2 for Lipid 31 LC-MS, and Table 5 for product mass).

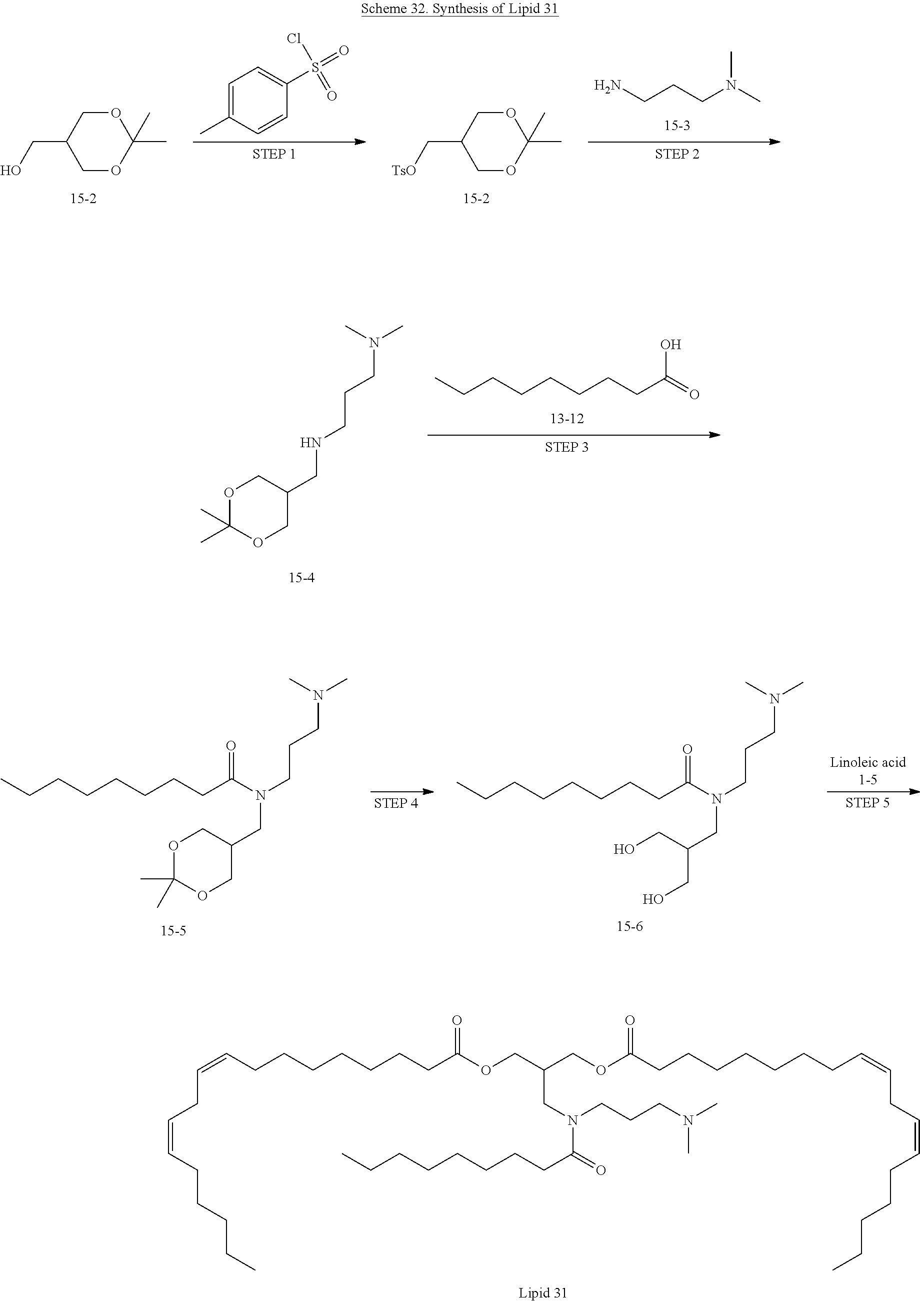

### Synthesis of Lipid 32

Lipid 32 was synthesized as provided in scheme 33 below and as follows. Intermediate 15-4 was produced as described for Lipid 31 above (steps 1 and 2, Scheme 30). N-acylation of 15-4 (4.34 mmol, 1 g, 1.0 eq) using 2-ethyl heptanoic acid 13-13 (10.85 mmol, 1.71 g, 2.5 eq) using EDCI (10.85 mmol, 2.07 g, 2.5 eq), DIEA (10.85 mmol, 1.40 g, 2.5 eq) and DMAP (10 mg) in 100 mL DCM yielded amine 15-7. Crude product was purified by column chromatography to obtain 724 mg (52%) of purified diamine 15-7. Diol intermediate 15-8 was accessed by deprotection of 15-7 (714 mg) in 3 mL of 1M HCl and 7 mL THF at room temperature for 1 hour in quantitative yield. Intermediate 15-8 (1.9 mmol, 630 mg, 1 eq) was acylated with linoleic acid 1-5 (6.49 mmol, 1.82 g, 3.4 eq) using EDCI (6.49 mmol, 1.23 g, 3.4 eq), DIPEA (6.49 mmol, 830 mg, 3.4 eq) and DMAP (20 mg, catalytic quantity) to obtain Lipid 32. Crude product was purified by column chromatography (4×) yielding 27 mg of pure fraction (>98% purity by LC-ELSD) of Lipid 32 and characterized by proton NMR and Mass Spectrometry (see FIG. 4S-1 for Lipid 32 NMR spectrum, FIG. 4S-2 for Lipid 32 LC-MS, and Table 5 for product mass).

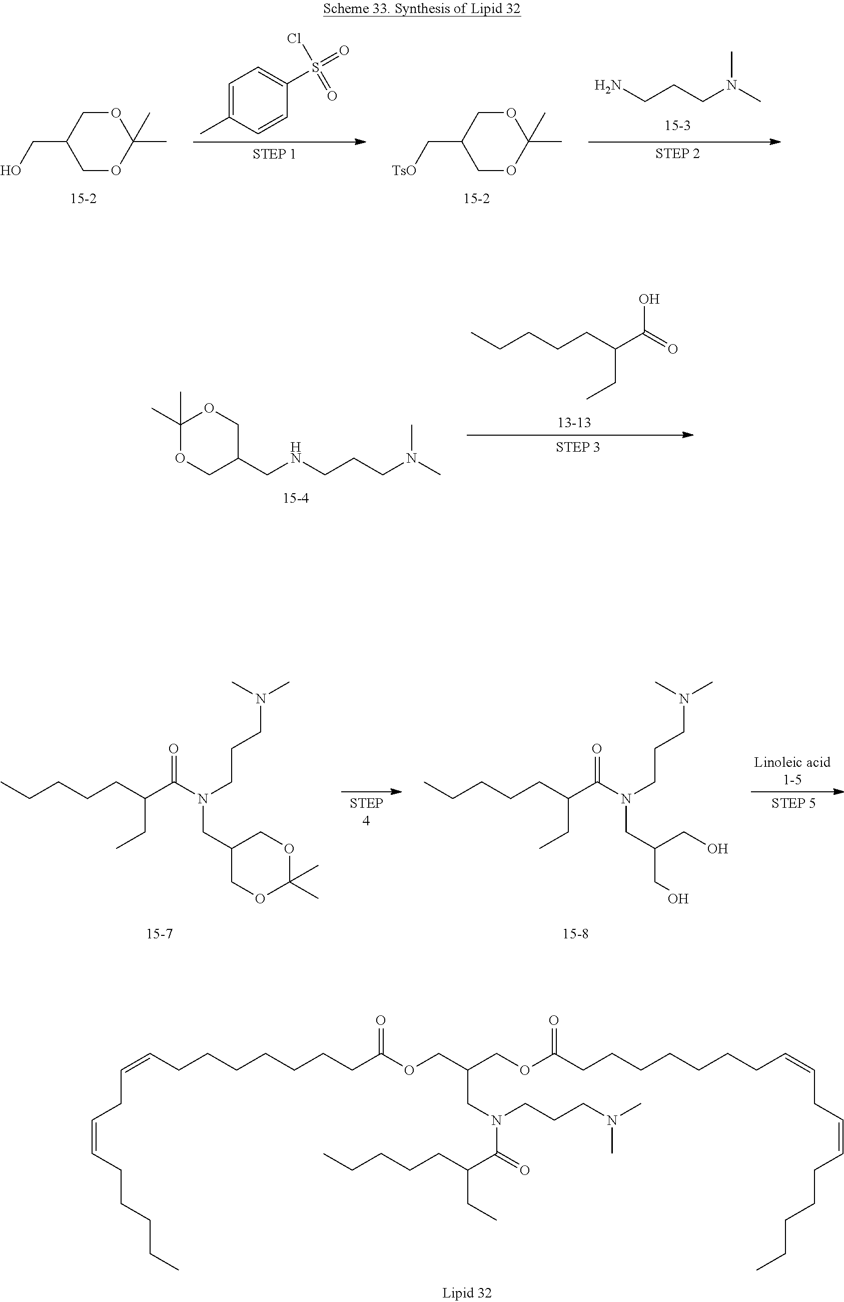

### Synthesis of Lipid 33

Lipid 33 was synthesized as provided in scheme 34 below and as follows. Starting material 15-1 (34.3 mmol, 5 g, 1 eq.) was tosylated using p-toluene sulfonyl chloride, TsCl (6.52 g, 1 eq, 34.3 mmol) using TEA (19.01 mL, 4 eq, 137 mmol) and DMAP (30 mg) in 200 mL DCM. Crude product was purified by column chromatography (1×) to obtain 10.2 g (98%) of reactive intermediate 15-2. Nucleophilic displacement of 15-2 (10.0 mmol, 2.3 g, 1 eq.) with diamine 15-9 (9.24 mmol, 1.0 mL, 1.2 eq.) in 10 mL dioxane (10 mL) yielded 1.6 g (97%) of compound 15-10. Nucleophilic displacement reaction was repeated using additional 15-2 (8.3 mmol, 2.5 g, 1 eq.) and diamine 15-9 (9.9 mmol, 1.1 mL, 1.2 eq.) in 50 mL dioxane for an additional quantity of compound 15-10. Crude products from the two reactions were purified by column chromatography to obtain a total of 1.7 g (~50%) pure 15-10. N-acylation of 15-10 (4.05 mmol, 875 mg, 1 eq.) with nonanoic acid 13-12 (7.1 mmol, 1.24 mL, 1.8 eq.) using EDCI (1.4 g, 1.8 eq, 7.1 mmol), DIPEA (1.3 mL, 1.8 eq, 7.1 mmol) and DMAP (90 mg, 0.2 eq, 0.81 mmol) in 8 mL DCM yielded intermediate 15-11. Crude product was purified by column chromatography (2×) to obtain 230 mg (16%) of pure intermediate 15-11. Deprotection of 15-11 (0.64 mmol, 230 mg, 1 eq.) in 5 mL of 4M HCl in dioxane yielded intermediate 15-12. Crude product was purified by column chromatography (1×) to obtain 74 mg (37%) of pure intermediate 15-12. Intermediate 15-12 (0.24 mmol, 74 mg, 1 eq.) was acylated with linoleic acid 1-5 (169 mg, 2.5 eq, 0.58 mmol) using EDCI (120 mg, 2.5 eq, 0.58 mmol), DIPEA (102 μL, 2.5 eq, 0.58 mmol) and DMAP (6 mg, 0.2 eq, 0.048 mmol) in 5 mL DCM to obtain Lipid 33. Crude product was purified by column chromatography (2×) to obtain 64 mg (32%) of pure Lipid 33 (>99% purity by LC-ELSD) and characterized by proton NMR and Mass Spectrometry (see FIG. 4T-1 for Lipid 33 NMR spectrum, FIG. 4T-2 for Lipid 33 LC-MS, and Table 5 for product mass).

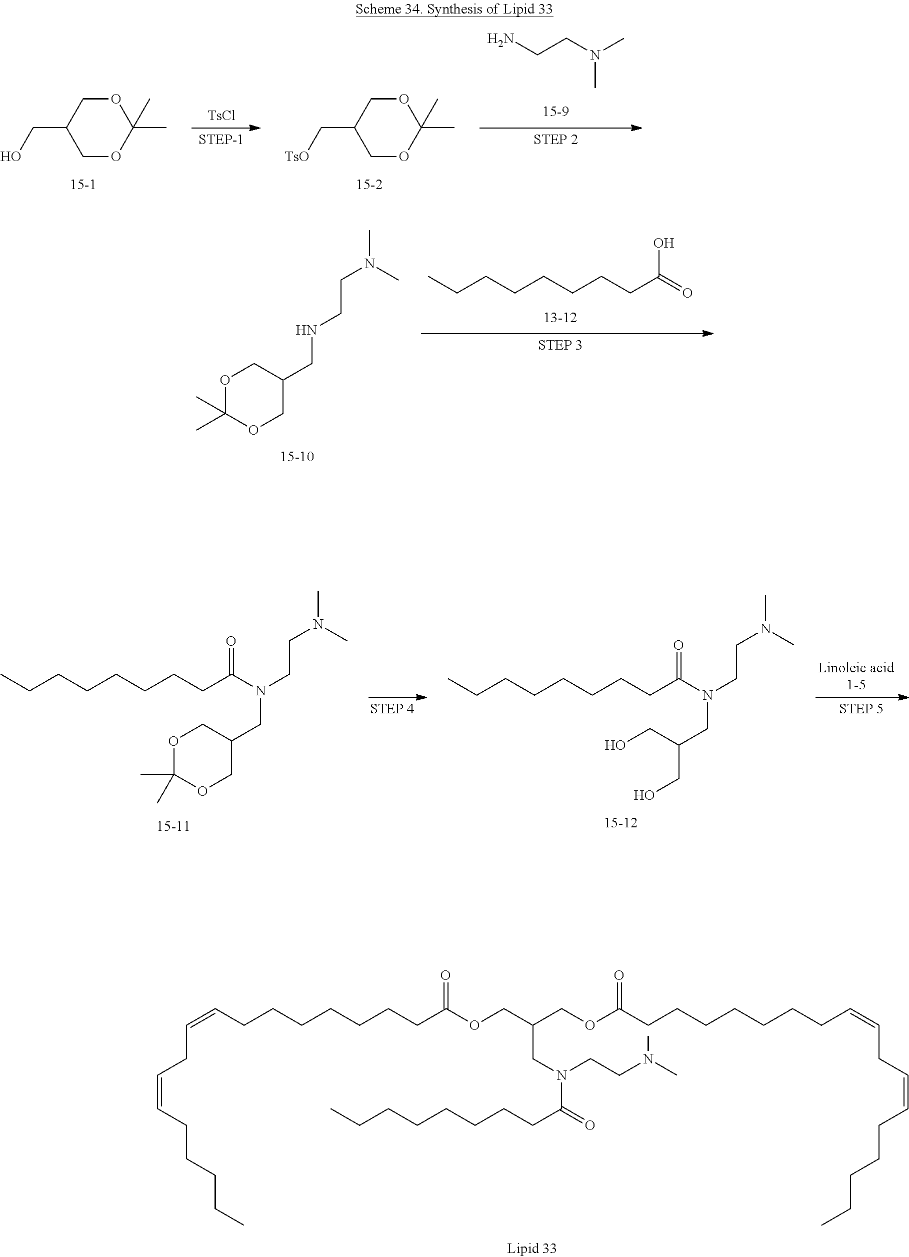

### Synthesis of Lipid 34

Lipid 34 was synthesized as provided in scheme 35 below and as follows. Intermediate 15-2 was accessed as described for Lipid 33. Nucleophilic displacement of 15-2 (3.3 mmol, 1 g, 1 eq.) with diamine 15-13 (3.9 mmol, 0.46 mL, 1.2 eq.) in 6 mL dioxane (10 mL) yielded 520 mg (64%) of compound 15-14. Reaction was repeated to access an additional 400 mg of pure compound 15-14. N-acylation of 15-14 (2.6 mmol, 620 mg, 1 eq.) with nonanoic acid 13-12 (5.3 mmol, 915 μL, 2.0 eq.) using EDCI (5.3 mmol, 1.06 g, 2.0 eq.), DIPEA (923 μL, 2.0 eq, 5.3 mmol) and DMAP (58 mg, 0.2 eq, 0.05 mmol) in 10 mL DCM yielded intermediate 15-15. Crude product was purified by column chromatography (2×) to obtain 355 mg (35%) of pure intermediate 15-15. Deprotection of 15-15 (1.03 mmol, 355 mg, 1 eq.) in 7 mL of 4M HCl in dioxane yielded intermediate 15-16. Crude product was purified by column chromatography (2×) to obtain 40 mg (13%) of pure intermediate 15-16. Intermediate 15-16 (0.116 mmol, 40 mg, 1 eq.) was acylated with linoleic acid 1-5 (81 mg, 2.5 eq, 0.29 mmol) using EDCI (55 mg, 2.5 eq, 0.29 mmol), DIPEA (3.2 μL, 2.5 eq, 0.29 mmol) and DMAP (2 mg, 0.2 eq, 0.05 mmol) in 10 mL DCM to obtain Lipid 34. Crude product was purified by column chromatography (2×) to obtain 73 mg (73%) of pure Lipid 34 (>99% purity by LC-ELSD) and characterized by proton NMR and Mass Spectrometry (see FIG. 4U-1 for Lipid 34 NMR spectrum, FIG. 4U-2 for Lipid 34 LC-MS, and Table 5 for product mass).

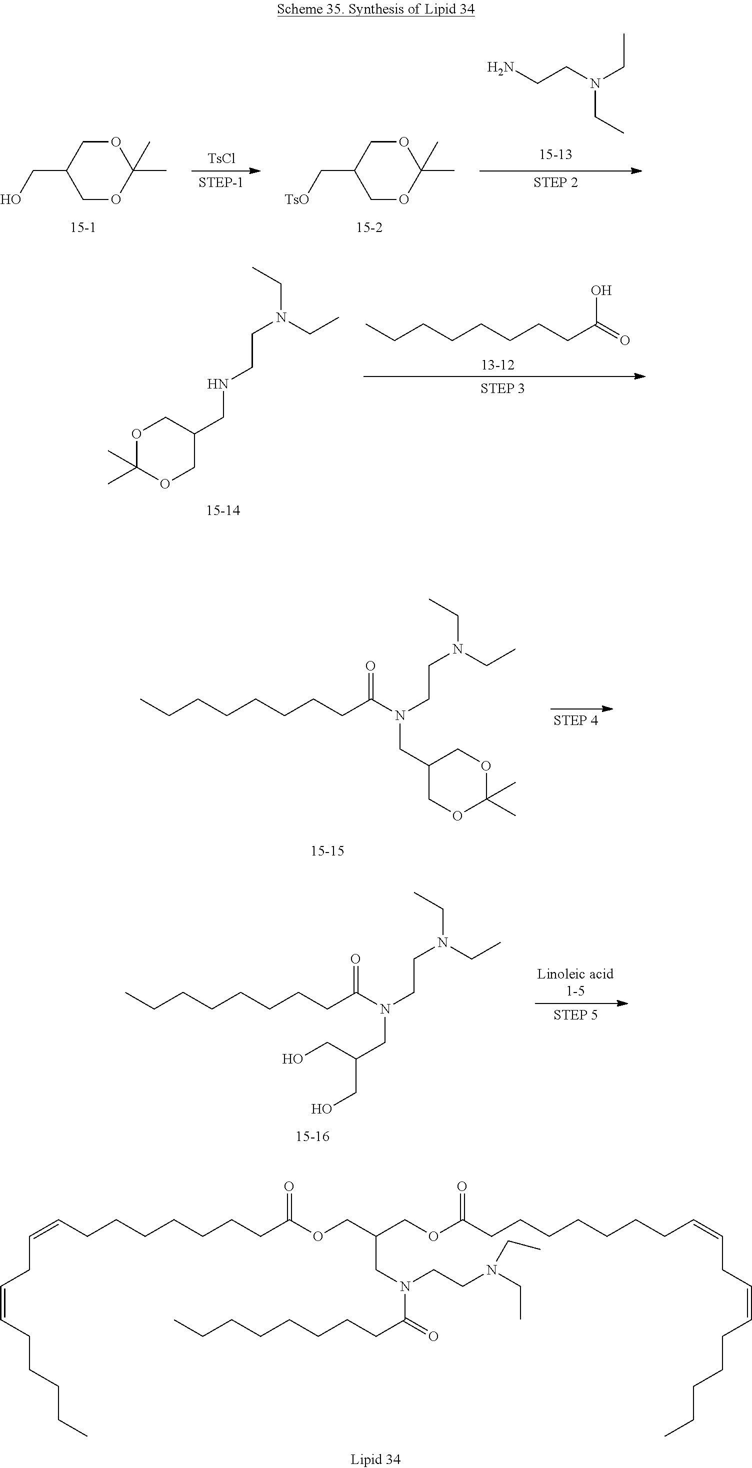

### Synthesis of Lipid 35

Lipid 35 was synthesized as provided in scheme 36 below.

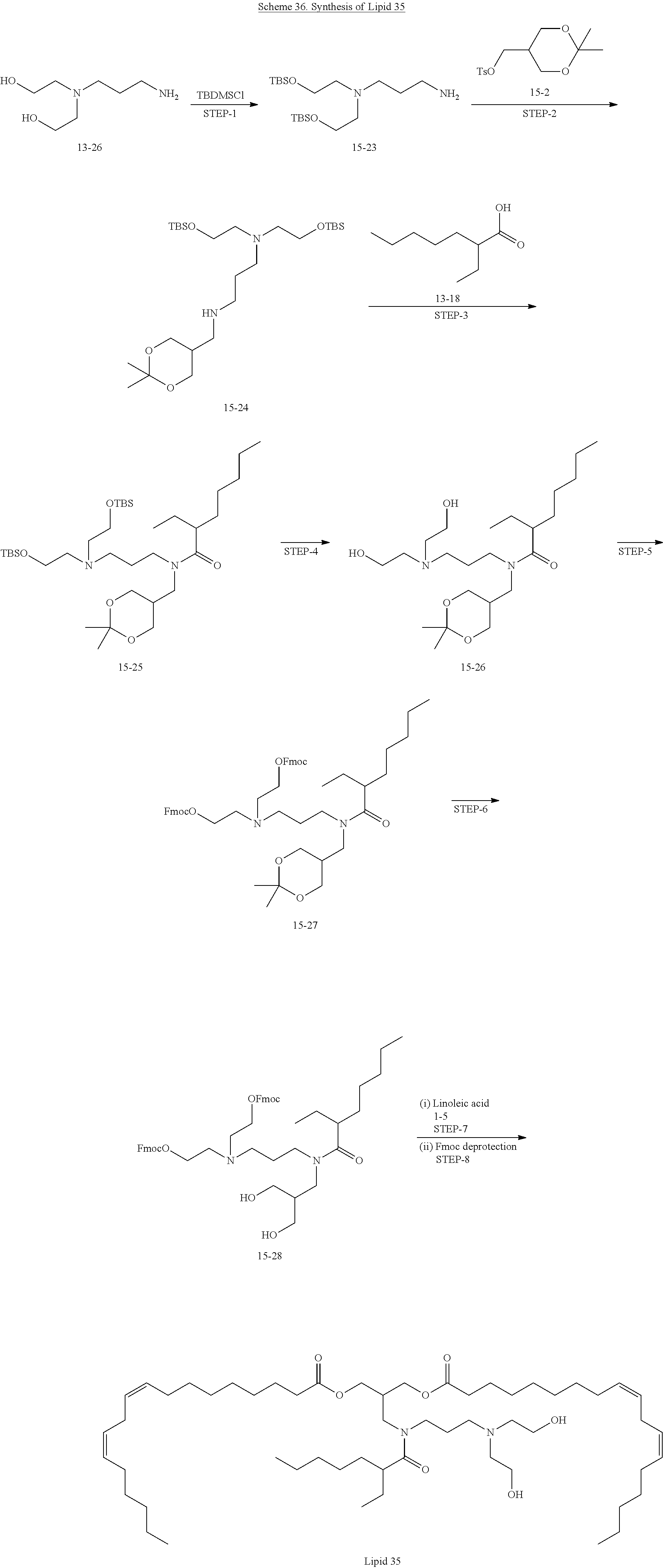

### Synthesis of Lipid 36

Lipid 36 was synthesized as provided in scheme 37 below.

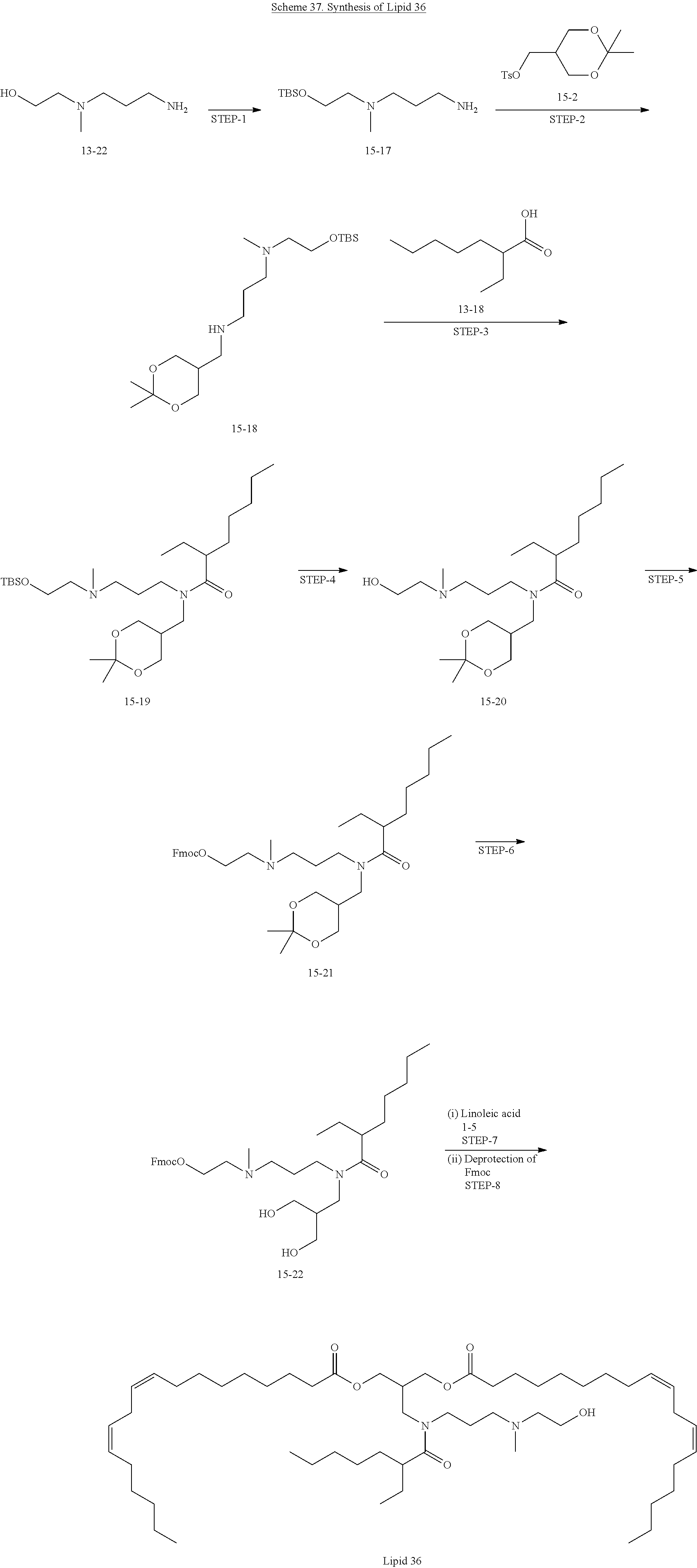

### Synthesis of Lipid 37A

Benzyl protected malonic acid 13-32 (5.1 mmol, 1.0 g, 1 eq.) was esterified with N-hexanol (0.78 g, 1.5 eq, 7.7 mmol) using HATU (2.93 g, 1.5 eq, 7.7 mmol) and DBU (1.56 g, 2.0 eq, 10.3 mmol) in 8 mL DMF for 16 hours at room temperature yielding 0.5 g (35%) of protected intermediate ester 14-9. A second 5 g scale batch using the same reaction conditions and reagent stoichiometry yielded an additional 6 g (84%) of intermediate 14-9. Intermediate 14-18 was produced via acylation of bromoacetyl bromide 13-35 with N-decanol.

Intermediate 14-9 (21.6 mmol, 6 g, 1 eq.) was alkylated with 14-18 (7.2 g, 1.2 eq, 26.0 mmol) using sodium hydride NaH (1.0 g, 1.2 eq, 26.0 mmol) in 40 mL DMF at room temperature overnight to obtain protected intermediate 14-19 (2.5 g, 25%). Intermediate 14-19 (2.5 g, 1.0 eq.) was deprotected using Pd/C (500 mg, 32% w/w) in 30 mL Ethyl Acetate at room temperature overnight yielding 2.02 g (99%) of acid intermediate 14-20.

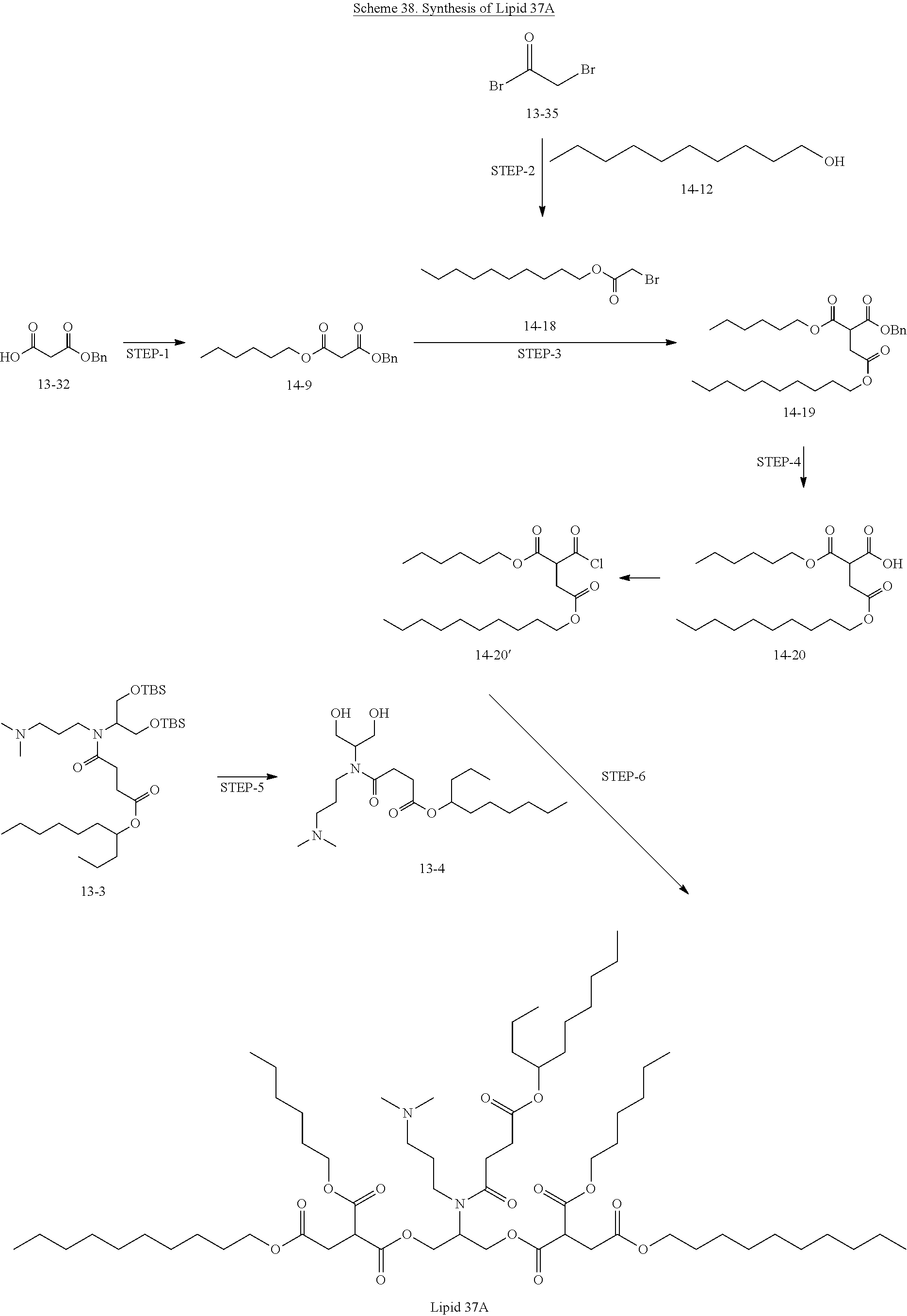

Acid intermediate 14-20 (2.7 mmol, 1.05 g, 1 eq.) was converted to the corresponding acid chloride 14-20′ using oxalyl chloride (0.79 mL, 3.4 eq, 9.2 mmol) and DMF (100 μL, catalytic quantity) in 6.0 mL of Toluene at room temperature for 2 hours and conversion to chloride intermediate was confirmed by TLC.

Intermediate 13-4 (1.57 mmol, 195 mg) was reacted with crude 14-20′ (2.83 g, 6.0 eq, 9.4 mmol) using TEA (2.18 mL, 10.0 eq, 15.7 mmol) in 16.0 mL of Toluene at room temperature overnight. Crude product was purified on ISCO column chromatography on silica column eluting with DCM and 10% MeOH in DCM twice to obtain 205 mg (38%) of >99% pure (HPLC-CAD) Lipid 37A. (see FIG. 4AH-1 and FIG. 4AH-2 for characterization by proton NMR and LC-CAD purity, and Table 5 for Mass Spectrometry data).

### Example 2. Preparation of LNPs by Microfluidic Mixing Using Exemplary Ionizable Lipids

Exemplary LNPs were produced using cationic Lipid 9 and cationic Lipid 15 as synthesized in Example 1 above.

LNPs encapsulating an mRNA payload were prepared by mixing an aqueous mRNA solution and an ethanolic lipid blend solution (containing ionizable lipid, DSPC, DPG-PEG and Cholesterol at lipid ratios shown in Table 6) using an in-line microfluidic mixing process. The mRNA (eGFP encoding mRNA, TriLink Biotechnologies, California, US) stock solution (1 mg/mL) was diluted in pH 4 acetate buffer (yielding a 133 μg/mL solution of mRNA) in 21.7 mM pH 4 acetate buffer. The lipid components were dissolved in anhydrous ethanol at the relative ratios set forth in Table 6 below.

TABLE 6
Components of exemplary lipid blend solution.

| | | Ratio of Lipid to | | Theoretical LNP |
|---|---|---|---|---|
| | | mRNA (nmol | Concentration in | Lipid |
| | | lipid/100 μg | Lipid Solution | Composition |
| Lipid | Source | mRNA) | (mM) | (mol %) |
| Ionizable Cationic | — | 1,500 | 6 | 49.2 |
| Lipid | | | | |
| Cholesterol | Dishman | 1,200 | 4.8 | 39.4 |
| | Netherlands | | | |
| DSPC | Avanti Polar | 300 | 1.2 | 9.8 |
| | Lipids, Alabama, | | | |
| | U.S. | | | |
| DPG-PEG(2000) | NOF America, | 46 | 0.18 | 1.5 |
| | New York, U.S. | | | |
| DiIC18(5)-DS | Invitrogen, | 1.8 | 0.007 | 0.06 |
| | Massachusetts, US | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directly mounted into the luer ports of the mixing cartridge. The two solutions were then mixed at a 3:1 v/v ratio of mRNA solution (1.5 mL) to lipid solution (0.5 mL) at a total flow rate of 9 mL/min using the NanoAssemblr Ignite. The resulting suspension was held at room temperature for a minimum of 5 minutes before proceeding to ethanol removal and buffer exchange.

Following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with HBS exchange buffer (25 mM HEPES buffer with 150 mM NaCl at pH 7.4). The LNP suspension (2 mL) was then loaded into the device and centrifuged at 500 RCF until the volume was reduced by half volume (1 mL). The suspension was then diluted with exchange buffer (1 mL, 25 mM HEPES buffer at pH 7.4) to bring the suspension back to the original volume. This process of two-fold concentration and two-fold dilution was repeated five additional times for a total of six discontinuous diafiltration steps. The LNP suspension was then exchanged into MBS (25 mM MES buffer with 150 mL NaCl at pH 6.5) by diluting ten-fold with MBS and centrifuging at 500 RCF until the volume was reduced by one tenth. This ten-fold dilution with MBS and ten-fold concentration step was repeated one more time. The retentate containing the LNPs in MBS was recovered from the centrifugal ultrafiltration device and stored at 4° C. until further use.

### Example 3. Characterization of LNPs

This Example describes the characterization of LNPs produced in Example 2 above.

Samples of the LNPs produced in Example 2 above were characterized to determine the average hydrodynamic diameter, zeta potential, and mRNA content (total and dye-accessible mRNA). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer.

RNA content of the nanoparticles was measured using Thermo Fisher Quant-iT RiboGreen RNA Assay Kit. Dye accessible RNA, which includes both un-encapsulated RNA and accessible RNA at the LNP surface, was measured by diluting the nanoparticles to approximately 1 μg/mL mRNA using HEPES buffered saline, and then adding Quant-iT reagent to the mixture. Total RNA content was measured by disrupting a nanoparticle suspension by dilution of the stock LNP batch (typically at ≥40 μg/mL RNA) in 0.5% Triton solution in HEPES buffered saline to obtain a 1 μg/mL RNA solution (final nominal concentration based on formulation input values) and subsequent heating at 60° C. for 30 minutes followed by addition of Quant-It reagent. RNA was quantified by measuring fluorescence at 485/535 nm, and concentration was determined relative to a contemporaneously run RNA standard curve. The results are set forth in Table 7.

TABLE 7
LNP formulation results.

| | | DLS | | Zeta | Zeta | Dye- |
|---|---|---|---|---|---|---|
| | | Z- | | Poten- | Poten- | Acces- |
| Form- | | Avg. | | tial at | tial at | sible |
| ulation | Ionizable | Diameter | DLS | pH 5.5 | pH 7.4 | mRNA |
| No. | Lipid | (nm) | PDI | (mV) | (mV) | (%) |
| 1 | Cationic Lipid 9 | 95 | 0.07 | 17 | 0.0 | 5 |
| 2 | Cationic Lipid 15 | 98 | 0.06 | 20 | 0.3 | 5 |

### Example 4. Preparation of Fab Conjugates to Enable T Cell Targeting

This Example describes the production of an exemplary lipid-immune cell targeting group conjugate.

An anti-CD3 Fab (hSP34 with mouse lambda and human lambda) (see amino acid sequences below) was conjugated to DSPE-PEG (2k)-maleimide via covalent coupling between the maleimide group and a C-terminal cysteine in the heavy chain (HC). Anti-CD3 Fab clone Hu291, anti-CD8 Fab clone TRX2, anti-CD8 Fab clone OKT8, a non-functional mutated OKT8 (mutOKT8), anti-CD4 Fab from Ibalizumab sequence, anti-CD5 Fab clone He3, anti-CD7 Fab clone TH-69, anti-CD2 Fab clone TS2/18.1, anti-CD2 Fab clone 9.6, anti-CD2 Fab clone 9-1 with human kappas were also conjugated using similar methods described herein. The protein (3-4 mg/mL), after buffer exchange into oxygen free, pH 7 phosphate buffer, was reduced in 2 mM TCEP in oxygen free pH 7 phosphate buffer for 1 hour at room temperature. The reduced protein was isolated using a 7 kDa SEC column to remove TCEP and buffer exchanged into fresh oxygen free pH 7 phosphate buffer.

The conjugation reaction was initiated by addition of a 10 mg/mL micellar suspension of DSPE-PEG-maleimide (Avanti Polar Lipids, Alabama, US) and 30 mg/mL DSPE-PEG-OCH₃ (Avanti Polar Lipids, Alabama, U.S.) (1:1 to 1:3 weight ratio is used depending on protein) in oxygen free pH 5.7 citrate buffer (1 mM Citrate). Protein solution is concentrated to 3-4 mg/mL using a 10 kDa Regenerated Cellulose Membrane and subsequently buffer exchanged in oxygen free pH 7 phosphate buffer using a 40 kDa Size Exclusion Column. The conjugation reaction is carried out using 2-4 mg/mL protein and a 3.5 molar excess of maleimide at 37° C. for 2 hours followed by incubation at room temperature for an additional 12-16 hours.

The production of the resulting conjugate was monitored by HPLC and the reaction quenched in 2 mM cysteine. The resulting conjugate (DSPE-PEG(2k)-anti-hSP34 Fab) is isolated using a 100 kDa Millipore Regenerated Cellulose membrane filtration using pH 7.4 HEPES buffer saline (25 mM HEPES, 150 mM NaCl) buffer and stored at 4° C. prior to use. After quenching, the final micelle composition consists of a mixture of DSPE-PEG-Fab, DSPE-PEG-maleimide (cysteine terminated), and DSPE-PEG-OCH₃. The ratio of the three components is DSPE-PEG-Fab:DSPE-PEG-maleimide (cysteine terminated):DSPE-PEG-OCH₃=1:2.45:3.45-10.35 (by mole)).

The resulting conjugate displayed comparable binding to recombinant Rhesus CD3 epsilon as the unconjugated anti-CD3 Fab by ELISA assay.

Sequences of antibodies in this example are provided in International Patent Application NO. PCT/US2023/068090, which is incorporated by reference in its entirety.

### Example 5. Preparation of LNPs Containing T Cell Targeting Group

This Example describes the incorporation of an immune cell targeting conjugate into a preformed LNP.

LNPs from Example 2 above, and conjugates (anti-CD3 (hSP34) and anti-CD8 (TRX-2) conjugates) prepared using methods described in Example 4 above, were combined as shown in Table 8 in an Eppendorf tube and vortexed for 10 seconds at 2,500 rpm. The Eppendorf tubes were placed in the ThermoMixer at 37° C. at 300 rpm for 4 hours. Resulting targeted LNP suspension was subsequently stored at 4° C. until use or alternatively stored frozen after reconstitution into sucrose medium at final sucrose concentration of 9.6 wt. % by dilution using the appropriate volume of a 50 wt. % sucrose stock solution (in HEPES buffer saline; 25 mM HEPES, 150 mM NaCl)) and stored frozen at −80° C.

TABLE 8
Results of combining exemplary LNPs and FAb conjugates.

| | Target | | | | | |
|---|---|---|---|---|---|---|
| | FAb | | | | | |
| | density | | | | | |
| | in | | RNA | FAb | | |
| nmol | targeted | | Conc. | Conj- | | |
| total | LNP g | FAb | In | ugate | Fab | LNP |
| lipid/ | (Fab/ | mg/ | LNP | Conc. | mg/ | mL/ |
| mg | mol | mg | (mg/ | (mg/ | mL | mL |
| RNA | lipid) | RNA | mL) | mL) | LNP | Fab |
| 30,460 | 9 | 0.274 | 0.45 | 1.46 | 0.123 | 11.8 |
| 30,460 | 3 | 0.091 | 0.45 | 1.46 | 0.041 | 35.5 |

### Example 6. Preparation of LNPs by Microfluidic in-Line Mixing and Tangential Flow Filtration Using an Exemplary Ionizable Lipid

This example describes preparation of LNPs using scalable unit operations, namely in-line microfluidic mixing followed by tangential flow filtration (TFF) for ethanol removal and buffer exchange. Separate LNP batches were prepared using either DLin-KC3-DMA or Lipid 15 as the ionizable lipid.

LNPs encapsulating an mRNA payload were prepared by mixing an aqueous mRNA solution and an ethanolic lipid blend solution using an in-line microfluidic mixing process. The mRNA (eGFP or mCherry encoding mRNA, TriLink Biotechnologies, California, US) stock solution was diluted in pH 4 acetate buffer (yielding a 400 μg/mL solution of mRNA) in 65 mM pH 4 acetate buffer. The lipid components were dissolved in anhydrous ethanol at the relative ratios set forth in Table 9.1 below.

TABLE 9.1
Ratios of dissolved LNP components.

| | | Ratio of Lipid to | | Theoretical LNP |
|---|---|---|---|---|
| | | mRNA (nmol | Concentration in | Lipid |
| | | lipid/100 μg | Lipid Solution | Composition |
| Lipid | Source | mRNA) | (mM) | (mol %) |
| Ionizable Cationic | — | 1,500 | 18.0 | 49.2 |
| Lipid | | | | |
| Cholesterol | Dishman | 1,200 | 14.4 | 39.4 |
| | Netherlands | | | |
| DSPC | Avanti Polar | 300 | 3.6 | 9.8 |
| | Lipids, Alabama, | | | |
| | U.S. | | | |
| DPG-PEG(2000) | NOF America, | 46 | 0.55 | 1.5 |
| | New York, U.S. | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directed mounted into the luer ports of the mixing cartridge. The two solutions were then mixed at a 3:1 v/v ratio of mRNA solution to lipid solution at a total flow rate of 9 mL/min using the NanoAssemblr Ignite. The resulting suspension was held at room temperature for a minimum of 5 minutes before proceeding to ethanol removal and buffer exchange. Ethanol removal and buffer exchange were subsequently performed using constant volume tangential flow filtration (TFF).

Following mixing, ethanol removal and buffer exchange were performed on the resulting LNP suspension using a hollow fiber TFF module (mPES membrane with 300 kDa MWCO, Repligen, US). Briefly, the TFF module was rinsed with DI water and pumped dry before use. The buffer selected for use as the diafiltration buffer depended on the ionizable lipid in the LNP formulation. For DLin-KC3-DMA LNPs, 25 mM pH 7.4 HEPES buffer with 150 mM NaCl (HBS) was used as the diafiltration buffer. For Lipid 15 LNPs, 25 mM pH 6.5 MES buffer with 150 mM NaCl (MBS) was used as the diafiltration buffer. The LNP mixture in ethanol/water solution was added to the reservoir, the TFF module was primed, and diafiltration was initiated by ramping up the peristaltic pump to the target flow rate and adjusting the retentate valve until the target transmembrane pressure (TMP) was reached. A shear rate of 8000 s⁻¹ and a TMP of 3.5 psi were the target operating parameters for the system once diafiltration was initiated. Throughout the diafiltration process, the TMP was kept constant by adjusting the retentate valve. Permeate flux was monitored and was >20 LMH throughout diafiltration. Six diafiltration exchange volumes were performed, with samples set aside at the end of each diafiltration volume to later track the buffer exchange process. Final ethanol content was ≤0.1%, as measured by refractive index measurements on permeate samples, and pH measurements confirmed the buffer exchange into the desired diafiltration buffer. Upon the completion of six diafiltration volumes, a concentration of the resulting LNP suspension was subsequently performed.

The concentration of the LNP suspension to a target total mRNA concentration of ~0.8 mg/mL was performed using the same TFF module that was used during the buffer exchange process. TMP and flow rate during the buffer exchange process were maintained, and the suspension was allowed to concentrate by stopping the addition of diafiltration buffer to the retentate reservoir. The resulting LNP suspension was collected and filtered with a 0.2 μm syringe filter. The suspension was sampled for analytical purposes and then stored at 4° C. until further use.

Using the LNP characterization process in Example 3 above, LNP batch was characterized to determine the average hydrodynamic diameter and mRNA content (total and dye-accessible), as set forth in Table 9.2 below. As seen in Table 9.2, the microfluidic mixing process with ethanol removal and buffer exchange by TFF results in sub-100 nm particles exhibiting narrow polydispersity and good mRNA encapsulation (<20% dye accessible RNA).

TABLE 9.2
Results of LNP microfluidic mixing process.

| | | | | | Dye- | |
|---|---|---|---|---|---|---|
| | | | | | Acces- | |
| | | DLS | | Total | sible | |
| | | Z- | | mRNA | mRNA | Dye- |
| | | Avg. | | Con- | Con- | Acces- |
| Sample | | Dia- | | tent | tent | sible |
| ID/Lot | | meter | DLS | (μg/ | (μg/ | mRNA |
| Number | Description | (nm) | PDI | mL) | mL) | (%) |
| EXP22001562- | DLin-KC3- | 85 | 0.07 | 846 | 115 | 14 |
| NF40 | DMA | | | | | |
| | LNPs after | | | | | |
| | 6 DVs | | | | | |
| | in HBS | | | | | |
| EXP22007940- | Lipid 15 | 69 | 0.14 | 990 | 161 | 16 |
| NU400 | LNPs | | | | | |
| | after 6 | | | | | |
| | DVs in | | | | | |
| | MBS | | | | | |

### Example 7. Method for Determination of the LNP Apparent PKa Using the Toluidinyl-Naphthalene Sulfonate (TNS) Fluorescent Probe

This example describes the fluorescent dye-based method used for measurement of the apparent pKa of the lipid nanoparticles. Apparent pKa determines the nanoparticle surface charge under physiological pH conditions, typically a pKa value in the endosomal pH range (6-7.4) results in LNPs that are neutral or slightly charged at plasma or the extracellular space (pH 7.4) and become strongly positive under acidic endosomal environments. This positive surface charge drives fusion of the LNP surface with negatively charged endosomal membranes resulting in destabilization and rupture of the endosomal compartment and LNP escape into the cytosolic compartment, a critical step in cytosolic delivery of mRNA and protein expression via engagement of the cells ribosomal machinery.

The apparent pKa of LNPs is determined by 6-(p-Toluidino)-2-naphthalenesulfonic acid (TNS) fluorescence measurement in aqueous buffers covering a range of pH values (pH 4-pH 10). TNS dye is non-fluorescent when free in solution, but fluoresces strongly when associated with a positively charged lipid nanoparticle. At pH values below the pKa of the nanoparticle, positive LNP surface charge results in dye recruitment at the particle interface resulting in TNS fluorescence. At pH values above the LNP pKa, the LNP surface charge is neutralized and TNS dye dissociates away from the particle interface resulting in loss of fluorescence signal. The apparent pKa of the LNP is reported as the pH at which the fluorescence is at 50% of its maximum, as determined using a four-point logistic curve fit.

### Example 8. General Formulation and Physiochemical Characterization Methodology for mRNA Encapsulating LNPs Based on Lipids 1-8, 9-15 and 31-34

Lipid nanoparticles (LNPs) bearing a nucleic acid (reporter RNA or CAR RNA) were formulated by a microfluidic mixing process using lipid and solvent compositions described in Example 2 and 6 above and buffer exchanged into pH 7.4 HEPES buffer saline (resulting in ethanol removal and pH adjustment) using either centrifugal ultrafiltration membrane filter devices or a tangential flow filtration (TFF) process; and characterized by Dynamic Light Scattering (DLS) for hydrodynamic size (diameter, nm), polydispersity (PDI) and charge at pH 5.5 and pH 7.4 (Zeta Potential, mV). The mRNA encapsulation efficiency (percent dye accessible RNA) and total mRNA content (ug/mL RNA in LNP suspension) were determined using methods described in Example 3 above. The formulated LNPs were subsequently buffer exchanged into pH 6.5 MES buffer saline and the size distribution was re-characterized by DLS prior to mixing with the desired quantity of targeting antibody conjugate (see Table 8 in Example 5 above) and incubated at 37° C. for 4 hours to facilitate antibody insertion (using process described in Example 5 above) resulting in final antibody targeted LNPs. The obtained targeted LNPs were sterile filtered and characterized by DLS (size (nm) and PDI) using methods described in Example 3 above.

### Example 9. Physiochemical Properties (Pre- and Post-Insertion with αCD3 Fab Conjugate Hsp34) of Lipids 1-8 GFP RNA Lipid Nanoparticles (LNPs)

Lipids 1, 2, 3, 4, 5, 6, 7 and 8 LNPs encapsulating either GFP-mRNA (TriLink Biotechnologies Inc.) or CAR-RNA (custom made by TriLink Biotechnologies Inc.) were prepared and characterized using methods described in Examples 5 to 8 above. Measured LNP size, PDI, charge and RNA content values for Lipid 1-8 LNP and LNPs made using comparator ionizable lipids (including MC3, KC2, SM-102, and ALC-0315) are summarized in Table 10 to Table 12. As seen in FIG. 5A, the initial mixing step and subsequent buffer exchange into HBS resulted in Lipid 1-8 LNP sizes in the 80-120 nm range. All lipids resulted in high encapsulation efficiency (<15% Dye accessible RNA) and >70% RNA recovery. The subsequent buffer exchange into pH 6.5 MES buffer and antibody insertion process (37° C., 4 hours) was well tolerated by lipids 2, 3, 6, 7 and 8 LNPs resulting in targeted LNP diameters below 140 nm and PDI<0.2. Lipids 1 and 4 LNPs exhibited relatively larger size shifts resulting in final targeted LNPs in the 140-160 nm diameter range, however particles size distributions remained narrow and monomodal for all lipids tested. Lipid 5 LNPs exhibited the greatest size shift with in final targeted LNPs exhibiting >160 nm diameter. Lipids 1, 2, 6, 7 and 8 exhibited no significant size and polydispersity changes after one Freeze-Thaw cycle, however, moderate changes were observed with LNPs based on lipids 3, 4, and 5. In summary, all lipids tested resulted in viable mRNA encapsulation and freeze-thaw stability as well as <200 nm final targeted LNP diameters. The ability of lipids 1-8 LNPs for inducing in vitro protein transfection in primary human T cells mediated by αCD3 or αCD8 T cell receptor targeting was evaluated as described in Examples 16 and 17 below.

TABLE 10
Lipids 1-8 and comparator Lipids LNP Size, Polydispersity (DLS) data in pH 7.4
HBS, pH 6.5 MBS and post αCD3 antibody Fab (hSP34) conjugate insertion.

| | | | Z-Avg. | | | | Polydis- | |
|---|---|---|---|---|---|---|---|---|
| | | | Size | Z-Avg. | | | persity | Polydis- |
| | Z-Avg. | Z-Avg. | (nm); | Size | Polydis- | Polydis- | (DLS); | persity |
| Ionizable | Size | Size | post- | (nm); | persity | persity | Post- | (DLS); |
| Lipid, LNP | (nm); | (nm); | insertion; | Post | (DLS); | (DLS); | insertion; | Post |
| no. | HBS | MBS | MBS | F/T | HBS | MBS | MBS | F/T |
| Lipid 1, | 98.78 | 124.3 | 146.2 | 145.5 | 0.08 | 0.128 | 0.11 | 0.14 |
| DPG-PEG; | | | | | | | | |
| EXP2200191 | | | | | | | | |
| 0-NL | | | | | | | | |
| Lipid 2, | 105.4 | 103.95 | 116.5 | 110.5 | 0.136 | 0.142 | 0.133 | 0.15 |
| DMG-PEG; | | | | | | | | |
| EXP2100218 | | | | | | | | |
| 2-N3 | | | | | | | | |
| Lipid 2, | 107 | 105 | 115.2 | 115.1 | 0.104 | 0.072 | 0.112 | 0.1 |
| DPG-PEG; | | | | | | | | |
| EXP2100218 | | | | | | | | |
| 2-N4 | | | | | | | | |
| Lipid 2, | 94 | 101.3 | 114.7 | 114.8 | 0.07 | 0.07 | 0.16 | 0.127 |
| DPG-PEG; | | | | | | | | |
| EXP2100234 | | | | | | | | |
| 0-N5 | | | | | | | | |
| Lipid 3, | 98.1 | 107.8 | 111.8 | | 0.11 | 0.1 | 0.095 | |
| DMG-PEG; | | | | | | | | |
| EXP2100347 | | | | | | | | |
| 1-N5 | | | | | | | | |
| Lipid 3, | 85.3 | 93.7 | 110.4 | | 0.07 | 0.08 | 0.16 | |
| DPG-PEG; | | | | | | | | |
| EXP2100347 | | | | | | | | |
| 1-N4 | | | | | | | | |
| Lipid 3, | 100.7 | 120.5 | 131.3 | 133.3 | 0.07 | 0.07 | 0.08 | 0.09 |
| DPG-PEG; | | | | | | | | |
| EXP2100234 | | | | | | | | |
| 0-N6 | | | | | | | | |
| Lipid 3, | 101.5 | 110.8 | 123.6 | 150.2 | 0.08 | 0.09 | 0.127 | 0.2 |
| DPG-PEG; | | | | | | | | |
| EXP2200191 | | | | | | | | |
| 0-NC | | | | | | | | |
| Lipid 4, | 79.5 | 96.3 | 116.8 | 128.5 | 0.04 | 0.11 | 0.105 | 0.18 |
| DPG-PEG; | | | | | | | | |
| EXP2100365 | | | | | | | | |
| 1-N6 | | | | | | | | |
| Lipid 4, | 95.5 | 120.6 | 155.4 | 170.3 | 0.09 | 0.13 | 0.137 | 0.17 |
| DPG-PEG; | | | | | | | | |
| EXP2200191 | | | | | | | | |
| 0-NI | | | | | | | | |

TABLE 11
Lipids 1-8 LNP charge, Zeta Potential, ZP (DLS, mV) in pH 5.5 MES
and pH 7.4 HBS.

| Ionizable Lipid, LNP no. | Charge (ZP, mV); pH 5.5 | Charge (ZP, mV); pH 7.4 |
|---|---|---|
| Lipid 1, DPG-PEG; EXP22001910-NL | 19.9 | −0.212 |
| Lipid 2, DMG-PEG; EXP21002182-N3 | 25.3 | 3.64 |
| Lipid 2, DPG-PEG; EXP21002182-N4 | 23.1 | 2.03 |
| Lipid 2, DPG-PEG; EXP21002340-N5 | 22.8 | 1.29 |
| Lipid 3, DMG-PEG; EXP21003471-N5 | 21.7 | 1.18 |
| Lipid 3, DPG-PEG; EXP21003471-N4 | 17.9 | 0.776 |
| Lipid 3, DPG-PEG; EXP21002340-N6 | 19.6 | −2.01 |
| Lipid 3, DPG-PEG; EXP22001910-NC | 23.9 | 3.99 |
| Lipid 4, DPG-PEG; EXP21003651-N6 | 19 | −0.508 |
| Lipid 4, DPG-PEG; EXP22001910-NI | 21.6 | 1.16 |
| Lipid 5, DPG-PEG; EXP22001910-NM | 19.1 | −0.742 |
| Lipid 6, DPG-PEG; EXP21003651-N7 | 13.5 | −1.9 |
| Lipid 7, DPG-PEG; EXP21003651-N8 | 10.8 | −4.31 |
| Lipid 8, DPG-PEG; EXP22002705-NO | 20.6 | 0.051 |
| SM-102, DPG-PEG; EXP21003651-N3 | 12.8 | −2.25 |
| ALC-0315, DPG-PEG; EXP21003651-N4 | 4.25 | −2.95 |
| MC-3, DPG-PEG; EXP21003651-N2 | 12.7 | −0.43 |
| KC-2, DPG-PEG; EXP21003651-N1 | 22.7 | 1.41 |

TABLE 12
Lipids 1-8 and comparator Lipids LNP Dye Accessible RNA
and total RNA content.

| | Nominal | | Dye- | Total | |
|---|---|---|---|---|---|
| | mRNA | Measured | accessible | mRNA | Dye- |
| Ionizable Lipid, | Conc. | Total mRNA | mRNA | Recovery | accessible |
| LNP no. | (μg/mL) | (μg/mL) | (μg/mL) | (%) | mRNA (%) |
| Lipid 1, DPG-PEG; | 100 | 75.5 | 8.9 | 75.5 | 11.8 |
| EXP22001910-NL | | | | | |
| Lipid 2, DMG- | 100 | 134.7 | 11.1 | 134.7 | 8.2 |
| PEG; | | | | | |
| EXP21002182-N3 | | | | | |
| Lipid 2, DPG-PEG; | 100 | 136.9 | 9.4 | 136.9 | 6.9 |
| EXP21002182-N4 | | | | | |
| Lipid 2, DPG-PEG; | 100 | 71.8 | 7.8 | 71.8 | 10.9 |
| EXP21002340-N5 | | | | | |
| Lipid 3, DMG- | 100 | 83.2 | 8.1 | 83.2 | 9.8 |
| PEG; | | | | | |
| EXP21003471-N5 | | | | | |
| Lipid 3, DPG-PEG; | 100 | 85.7 | 6.6 | 85.7 | 7.7 |
| EXP21003471-N4 | | | | | |
| Lipid 3, DPG-PEG; | 100 | 93.5 | 10.3 | 93.5 | 11.0 |
| EXP21002340-N6 | | | | | |
| Lipid 3, DPG-PEG; | 100 | 74.9 | 6.5 | 74.9 | 8.7 |
| EXP22001910-NC | | | | | |
| Lipid 4, DPG-PEG; | 50 | 37.1 | 4.3 | 74.2 | 11.5 |
| EXP21003651-N6 | | | | | |
| Lipid 4, DPG-PEG; | 100 | 77.6 | 9 | 77.6 | 11.6 |
| EXP22001910-NI | | | | | |
| Lipid 5, DPG-PEG; | 100 | 73.4 | 11.8 | 73.4 | 16.1 |
| EXP22001910-NM | | | | | |
| Lipid 6, DPG-PEG; | 50 | 34.7 | 3.3 | 69.4 | 9.5 |
| EXP21003651-N7 | | | | | |
| Lipid 7, DPG-PEG; | 50 | 38.9 | 4.3 | 77.8 | 11.1 |
| EXP21003651-N8 | | | | | |
| Lipid 8, DPG-PEG; | 100 | 85.9 | 8.4 | 85.9 | 9.8 |
| EXP22002705-NO | | | | | |
| SM-102, DPG- | 50 | 28.7 | 4.3 | 57.4 | 15.0 |
| PEG; | | | | | |
| EXP21003651-N3 | | | | | |
| ALC-0315, DPG- | 50 | 38.1 | 4.8 | 76.2 | 12.6 |
| PEG; | | | | | |
| EXP21003651-N4 | | | | | |
| MC-3, DPG-PEG; | 50 | 36.8 | 1.8 | 73.6 | 4.9 |
| EXP21003651-N2 | | | | | |
| KC-2, DPG-PEG; | 50 | 43 | 3.6 | 86 | 8.4 |
| EXP21003651-N1 | | | | | |

### Example 10. Physiochemical Properties (Pre- and Post-Insertion with αCD3 Fab Conjugate HSP34) of Lipids 9, 10, 11 and 15 GFP RNA Lipid Nanoparticles (LNPs)

Lipids 9, 10, 11, and 15 LNPs encapsulating either GFP-mRNA (TriLink Biotechnologies Inc.) or CAR-RNA (custom made by TriLink Biotechnologies Inc.) were prepared and characterized using methods described in Examples 5 to 8 above. Measured LNP size, PDI, charge and RNA content values of resulting and of LNPs made using comparator ionizable lipids (including MC3, KC2, SM-102, and ALC-0315) are summarized in Table 13 to Table 15. As seen in FIG. 6A, the initial mixing step and subsequent buffer exchange into HBS resulted LNP sizes ≤100 nm with Lipids 9, 10, 11 and 15. All lipids resulted in high encapsulation efficiency (<15% Dye accessible RNA) and >70% RNA recovery. The subsequent buffer exchange into pH 6.5 MES buffer and antibody insertion process (37C, 4 hours) was well tolerated by lipids 9, 10, and 15 LNPs resulting in final targeted LNP diameters below 140 nm and PDI<0.2. Lipid 11 LNPs exhibited the greatest size shift upon buffer exchange into pH 6.5 MES buffer, and a moderately larger size shift upon antibody insertion relative to the other lipids tested. All four tested lipid LNPs were stable to one freeze-thaw cycle with no significant shifts in LNP diameter or polydispersity observed. In summary, all four lipids tested resulted in viable mRNA encapsulation and freeze-thaw stability as well as <200 nm final targeted LNP diameters. The ability of lipids 9, 10, 11 and 15 LNPs for inducing in vitro protein transfection in primary human T cells mediated by αCD3 or αCD8 T cell receptor targeting was evaluated as described in Examples 16 and 17 below.

TABLE 13
Lipids 9,10,11,15 LNPs and comparator Lipids LNP Size, Polydispersity (DLS) data
in pH 7.4 HBS, pH 6.5 MBS and post αCD3 antibody Fab (hSP34) conjugate insertion.

| | | | Z- | | | | | |
|---|---|---|---|---|---|---|---|---|
| | | | Avg. | Z- | | | | |
| | Z- | Z- | Size | Avg. | | | PDI | |
| | Avg. | Avg. | (nm); | Size | | | (DLS); | PDI |
| | Size | Size | post- | (nm); | PDI | PDI | Post- | (DLS); |
| Ionizable Lipid, | (nm); | (nm); | insertion; | Post | (DLS); | (DLS); | insertion; | Post |
| LNP no. | HBS | MBS | MBS | F/T | HBS | MBS | MBS | F/T |
| Lipid 9; | 87.6 | 102.4 | 118.9 | 125.3 | 0.09 | 0.11 | 0.11 | 0.15 |
| EXP21004287- | | | | | | | | |
| N2 | | | | | | | | |
| Lipid 9; | 83.9 | 94.8 | 105.1 | 115.1 | 0.08 | 0.07 | 0.12 | 0.13 |
| EXP22001910- | | | | | | | | |
| NE | | | | | | | | |
| Lipid 10; | 98.6 | 110.6 | 119.9 | 121.5 | 0.1 | 0.11 | 0.12 | 0.11 |
| EXP22002705- | | | | | | | | |
| NQ | | | | | | | | |
| Lipid 11; | 101.9 | 154.6 | 174.5 | 162.9 | 0.14 | 0.14 | 0.11 | 0.12 |
| EXP22002705- | | | | | | | | |
| NN | | | | | | | | |
| Lipid 15; | 91.55 | 97.65 | 114.8 | 118.3 | 0.07 | 0.06 | 0.13 | 0.17 |
| EXP22001910- | | | | | | | | |
| NP | | | | | | | | |
| SM-102; | 68.4 | 102.9 | 125.7 | 125.6 | 0.06 | 0.11 | 0.13 | 0.12 |
| EXP21003651- | | | | | | | | |
| N3 | | | | | | | | |
| ALC-0315; | 76.5 | 103.9 | 116.7 | 152.2 | 0.06 | 0.09 | 0.17 | 0.18 |
| EXP21003651- | | | | | | | | |
| N4 | | | | | | | | |
| DLin-MC3- | 72.2 | 92.6 | 124.8 | 123.65 | 0.07 | 0.07 | 0.16 | 0.15 |
| DMA; | | | | | | | | |
| EXP21003651- | | | | | | | | |
| N2 | | | | | | | | |
| DLin-KC2- | 68.8 | | 93.5 | 116.8 | 0.06 | | 0.18 | 0.36 |
| DMA; | | | | | | | | |
| EXP21003651- | | | | | | | | |
| N1 | | | | | | | | |

TABLE 14
Lipids 9, 10, 11, 15 Lipid and comparator Lipid LNP charge, Zeta Potential, ZP
(DLS, mV) in pH 5.5 MES and pH 7.4 HBS.

| | LNP charge (ZP, mV); pH | LNP charge (ZP, mV); pH |
|---|---|---|
| Ionizable Lipid, LNP no. | 5.5 | 7.4 |
| Lipid 9; EXP21004287-N2 | 10.1 | −2.34 |
| Lipid 9; EXP22001910-NE | 17.2 | 0.0437 |
| Lipid 10; EXP22002705-NQ | 21 | −0.137 |
| Lipid 11; EXP22002705-NN | 16.5 | −1.01 |
| Lipid 15; EXP22001910-NP | 19.7 | −0.322 |
| SM-102; EXP21003651-N3 | 12.8 | −2.25 |
| ALC-0315; EXP21003651-N4 | 4.25 | −2.95 |
| DLin-MC3-DMA; EXP21003651- | 12.7 | −0.43 |
| N2 | | |
| DLin-KC2-DMA; EXP21003651- | 22.7 | 1.41 |
| N1 | | |

TABLE 15
Lipids 9, 10, 11, 15 LNP and comparator Lipids LNP Dye Accessible RNA and
total RNA content.

| | Nominal | | Dye- | Total | |
|---|---|---|---|---|---|
| | mRNA | Measured | accessible | mRNA | |
| Ionizable Lipid, LNP | Conc. | Total mRNA | mRNA | Recovery | Dye-accessible |
| no. | (μg/mL) | (μg/mL) | (μg/mL) | (%) | mRNA (%) |
| Lipid 9; EXP21004287- | 100 | 72.3 | 7.5 | 72.3 | 10.4 |
| N2 | | | | | |
| Lipid 9; EXP22001910- | 100 | 84.4 | 5.4 | 84.4 | 6.4 |
| NE | | | | | |
| Lipid 10; | 100 | 80.2 | 6.6 | 80.2 | 8.2 |
| EXP22002705-NQ | | | | | |
| Lipid 11; | 100 | 85.6 | 13.6 | 85.6 | 15.9 |
| EXP22002705-NN | | | | | |
| Lipid 15; | 100 | 74.9 | 5.1 | 74.9 | 6.8 |
| EXP22001910-NP | | | | | |

### Example 11. Physiochemical Properties (Pre- and Post-Insertion with αCD3 Fab Conjugate Hsp34) of Lipids 31-34 GFP RNA Lipid Nanoparticles (LNPs)

Lipids 31, 32, 33, and 34 LNPs encapsulating either GFP-mRNA (TriLink Biotechnologies Inc.) or CAR-RNA (custom made by TriLink Biotechnologies Inc.) were prepared and characterized using methods described in Examples 5 to 8 above. Measured LNP size, PDI, charge and RNA content values of Lipids 31, 32, 33, and 34 LNPs are summarized in Table 16 to Table 18. As seen in FIG. 7A, the initial mixing step and subsequent buffer exchange into HBS resulted in LNP sizes ≤110 nm with Lipids 31, 32, 33, and 34. All LNPs exhibited moderate to high encapsulation efficiency (<20% Dye accessible RNA) and >70% RNA recovery. The subsequent buffer exchange into pH 6.5 MES buffer and antibody insertion process (37° C., 4 hours) resulted in final targeted LNP diameters in the 120 nm to 160 nm range and PDI in the 0.1-0.25 range indicating relatively poorer particle size control in lipids 31 through 34 LNPs. Lipid 31, 32, and 34 LNPs showed moderate size increases after one freeze-thaw cycle, however, a notably larger size shift was observed with lipid 34 LNPs. In summary, all four lipids tested resulted in viable mRNA encapsulation and freeze-thaw stability as well as <200 nm final targeted LNP diameters. The ability of lipids 31, 32, 33, 34 LNPs for inducing in vitro protein transfection in primary human T cells mediated by αCD3 or αCD8 T cell receptor targeting was evaluated as described in Examples 16 and 17 below.

TABLE 16
Lipids 31,32,33,34 LNPs and comparator Lipids LNP Size, Polydispersity (DLS)
data in pH 7.4 HBS, pH 6.5 MBS and post αCD3 antibody Fab (hSP34) conjugate insertion.

| | | | Z-Avg. | Z- | | | Polydis- | |
|---|---|---|---|---|---|---|---|---|
| | Z- | Z- | Size | Avg. | | | persity | Polydis- |
| Ionizable | Avg. | Avg. | (nm); | Size | Polydis- | Polydis- | (DLS); | persity |
| lipid, | Size | Size | post- | (nm); | persity | persity | Post- | (DLS); |
| LNP | (nm); | (nm); | insertion; | Post | (DLS); | (DLS); | insertion; | Post |
| no. | HBS | MBS | MBS | F/T | HBS | MBS | MBS | F/T |
| Lipid 31, | 77.1 | 75.2 | 159.8 | 171.7 | 0.14 | 0.12 | 0.21 | 0.25 |
| DMG-PEG; | | | | | | | | |
| EXP210020 | | | | | | | | |
| 02-N15B | | | | | | | | |
| Lipid 31, | 79.23 | 94.3 | 108.3 | ** | 0.22 | 0.18 | 0.17 | |
| DMG-PEG; | | | | | | | | |
| EXP210021 | | | | | | | | |
| 79-N15B | | | | | | | | |
| Lipid 32, | 78.3 | 101.4 | 149.9 | ** | 0.26 | 0.17 | 0.23 | |
| DMG-PEG; | | | | | | | | |
| EXP210021 | | | | | | | | |
| 79-N15C | | | | | | | | |
| Lipid 33, | 94.74 | 103.9 | 134.8 | 167.8 | 0.14 | 0.09 | 0.09 | 0.1 |
| DPG-PEG; | | | | | | | | |
| EXP210042 | | | | | | | | |
| 87-N3 | | | | | | | | |
| Lipid 33, | 92.2 | 94.1 | 98.085 | 125.3 | 0.21 | 0.1 | 0.10 | 0.14 |
| DPG-PEG; | | | | | | | | |
| EXP210047 | | | | | | | | |
| 81-N6 | | | | | | | | |
| Lipid 34, | 97.31 | 113.3 | 155.1 | 207.5 | 0.093 | 0.07 | 0.13 | 0.14 |
| DPG-PEG; | | | | | | | | |
| EXP210042 | | | | | | | | |
| 87-N4 | | | | | | | | |

** Not measured.

TABLE 17
Lipids 31, 32, 33, 34 Lipid and comparator Lipid LNP charge, Zeta Potential,
ZP (DLS, mV) in pH 5.5 MES and pH 7.4 HBS.

| | Charge (ZP, | Charge (ZP, |
|---|---|---|
| Ionizable lipid, LNP no. | mV); pH 5.5 | mV); pH 7.4 |
| Lipid 31, DMG-PEG; EXP21002002-N15B | 20.5 | −0.138 |
| Lipid 31, DMG-PEG; EXP21002179-N15B | | |
| Lipid 32, DMG-PEG; EXP21002179-N15C | | |
| Lipid 33, DPG-PEG; EXP21004287-N3 | 19.7 | −0.867 |
| Lipid 33, DPG-PEG; EXP21004781-N6 | 19.6 | 1.5 |
| Lipid 34, DPG-PEG; EXP21004287-N4 | 15 | −1.79 |

TABLE 18
Lipids 31, 32, 33, 34 LNP and comparator Lipids LNP Dye Accessible
RNA and total RNA content.

| | Nominal | Measured | Dye- | Total | Dye- |
|---|---|---|---|---|---|
| | mRNA | Total | accessible | mRNA | accessible |
| Ionizable lipid, | Conc. | mRNA | mRNA | Recovery | mRNA |
| LNP no. | (μg/mL) | (μg/mL) | (μg/mL) | (%) | (%) |
| Lipid 31, DMG-PEG; | 50 | 42.7 | 3.7 | 85.4 | 8.7 |
| EXP21002002-N15B | | | | | |
| Lipid 31, DMG-PEG; | 50 | 52.13 | 2.2 | 104.26 | 4.2 |
| EXP21002179-N15B | | | | | |
| Lipid 32, DMG-PEG; | 50 | 53.02 | 7.5 | 106.04 | 14.1 |
| EXP21002179-N15C | | | | | |
| Lipid 33, DPG-PEG; | 100 | 81.2 | 14.3 | 81.2 | 17.6 |
| EXP21004287-N3 | | | | | |
| Lipid 33, DPG-PEG; | 150 | 116.4 | 12.8 | 77.6 | 110 |
| EXP21004781-N6 | | | | | |
| Lipid 34, DPG-PEG; | 100 | 70 | 10.5 | 70 | 15 |
| EXP21004287-N4 | | | | | |

### Example 12. Physiochemical Properties (Pre- and Post-Insertion with αCD8 Fab Conjugates TRX-2 and 15C01) of Lipids 1, 3, 4, 5, 9 and 15 GFP RNA Containing Lipid Nanoparticles (LNPs)

Lipids 1, 3, 4, 5, 9 and 15 LNPs encapsulating GFP-RNA (custom made by TriLink Biotechnologies Inc.) were prepared and characterized using methods described in Examples 5 to 8 above. Measured LNP size, and PDI, Lipids 1, 3, 4, 5, 9 and 15 LNPs in both pre- and post-insertion with αCD8 fab (TRX-1 and 15C01) conjugates are summarized in Table 19. As seen in FIG. 8A, the initial mixing step and subsequent buffer exchange into HBS resulted in LNP sizes ≤120 nm with Lipids 3, 9 and 15, while Lipid 5 LNPs exhibited >150 nm diameter upon buffer exchange into pH 6.5 MBS. Lipids 9 and 15 LNPs exhibited the smallest sizes (≤120 nm) after both αCD8 antibody conjugate insertions (TRX-2 and 15C01) and post one freeze-thaw cycle. Similarly, polydispersity of all final αCD8 targeted LNPs remained <0.2 both pre- and post-freeze thaw (FIG. 8B). The ability of lipids 1, 3, 4, 5, 9 and 15 LNPs for inducing in vitro protein transfection in primary human T cells mediated by αCD8 T cell receptor targeting antibodies (TRX-2 and 15C01) was evaluated as described in Examples 18 and 19 below.

TABLE 19
Lipid 1,3,4,5,9, and 15 LNP size and PDI pre- and post- insertion with αCD8
(TRX-1 and 15C01) targeting Fab conjugate.

| | | | Z-Avg. | Z-Avg. | | | PDI | PDI |
|---|---|---|---|---|---|---|---|---|
| | Z- | Z- | Size (nm); | Size (nm); | | | (DLS); | (DLS); |
| | Avg. | Avg. | TRX-2 | 15C01 | | | TRX-2 | 15C01; |
| Ionizable | Size | Size | post- | post- | PDI | PDI | Post- | Post- |
| Lipid, | (nm); | (nm); | insertion; | insertion; | (DLS); | (DLS); | insertion; | insertion; |
| LNP no. | HBS | MBS | MBS | MBS | HBS | MBS | MBS | MBS |
| Lipid 3, DPG- | 101.5 | 110.8 | 117.4 | 131.0 | 0.08 | 0.09 | 0.116 | 0.166 |
| PEG, TRX-2; | | | | | | | | |
| EXP22001910- | | | | | | | | |
| NC | | | | | | | | |
| Lipid 9,DPG- | 83.9 | 94.8 | 102.5 | 108.1 | 0.08 | 0.07 | 0.1255 | 0.143 |
| PEG, TRX-2; | | | | | | | | |
| EXP22001910- | | | | | | | | |
| NE | | | | | | | | |
| Lipid 4, DPG- | 95.5 | 120.6 | 127.8 | 133.7 | 0.09 | 0.13 | 0.1325 | 0.114 |
| PEG, TRX-2; | | | | | | | | |
| EXP22001910- | | | | | | | | |
| NI | | | | | | | | |
| Lipid 1, DPG- | 98.78 | 124.25 | 130.2 | 133.7 | 0.08 | 0.128 | 0.108 | 0.123 |
| PEG, TRX-2; | | | | | | | | |
| EXP22001910- | | | | | | | | |
| NL | | | | | | | | |
| Lipid 5, DPG- | 102.4 | 161.2 | 162.3 | 161.3 | 0.09 | 0.13 | 0.1335 | 0.095 |
| PEG, TRX-2; | | | | | | | | |
| EXP22001910- | | | | | | | | |
| NM | | | | | | | | |
| Lipid 15; DPG- | 91.55 | 97.65 | 106.0 | 115.1 | 0.07 | 0.06 | 0.1415 | 0.164 |
| PEG, TRX-2; | | | | | | | | |
| EXP22001910- | | | | | | | | |
| NP | | | | | | | | |

### Example 13. Physiochemical Properties (Pre- and Post-Insertion with αCD8 (TRX-2) Fab Conjugate) of Lipids 1, 8, 9, 10, 11 and 15 GFP RNA Containing Lipid Nanoparticles (LNPs)

Lipids 1, 8, 9, 10, 11 and 15 LNPs encapsulating GFP-RNA (custom made by TriLink Biotechnologies Inc.) were prepared and characterized using methods described in Examples 5 to 8 above. Measured LNP size, and PDI, Lipids 1, 8, 9, 10, 11 and 15 LNPs in both pre- and post-insertion with αCD8 (TRX-2) Fab conjugate are summarized in Table 20. As seen in FIG. 9A, the initial mixing step and subsequent buffer exchange into HBS resulted in LNP sizes <130 nm with Lipids 8, 9, 10, 11 and 15, while Lipid 1 LNPs exhibited >150 nm diameter upon buffer exchange into pH 6.5 MBS. Lipids 8, 9, 10 and 15 LNPs exhibited the smallest sizes (≤130 nm) after both αCD8 antibody conjugate insertions (TRX-2 and 15C01) and post one freeze-thaw cycle. Polydispersity of all final &CD8 targeted LNPs remained <0.2 both pre- and post-freeze thaw (FIG. 9B). The ability of lipids 1, 8, 9, 10, 11 and 15 LNPs for inducing in vitro protein transfection in primary human T cells mediated by αCD8 T cell receptor targeting antibodies (TRX-2 and 15C01) was evaluated as described in Examples 16 and 17 below.

TABLE 20
Lipid 1,8,9,10,11 and 15 LNP size and PDI pre- and post-
insertion with αCD8 (TRX-2) targeting Fab conjugate.

| | | | Z- | | | |
|---|---|---|---|---|---|---|
| | | | Avg. | | | |
| | | | Size | | | PDI |
| | Z- | Z- | (nm); | | | (DLS); |
| | Avg. | Avg. | TRX-2 | | | TRX-2 |
| Ionizable | Size | Size | post-in- | PDI | PDI | Post-in- |
| Lipid, | (nm); | (nm); | sertion; | (DLS); | (DLS); | sertion; |
| LNP no. | HBS | MBS | MBS | HBS | MBS | MBS |
| Lipid 11, | 101.5 | 110.8 | 155.45 | 0.08 | 0.09 | 0.13 |
| DPG-PEG, | | | | | | |
| TRX-2; | | | | | | |
| EXP22002705- | | | | | | |
| NN | | | | | | |
| Lipid 8, | 83.9 | 94.8 | 110 | 0.08 | 0.07 | 0.13 |
| DPG-PEG, | | | | | | |
| TRX-2; | | | | | | |
| EXP22002705- | | | | | | |
| NO | | | | | | |
| Lipid 10, | 95.5 | 120.6 | 120.65 | 0.09 | 0.13 | 0.13 |
| DPG-PEG, | | | | | | |
| TRX-2; | | | | | | |
| EXP22002705- | | | | | | |
| NQ | | | | | | |
| Lipid 9, | 98.78 | 124.25 | 114.45 | 0.08 | 0.128 | 0.14 |
| DPG-PEG, | | | | | | |
| TRX-2; | | | | | | |
| EXP22001910- | | | | | | |
| NE | | | | | | |
| Lipid 1, | 102.4 | 161.2 | 142.75 | 0.09 | 0.13 | 0.14 |
| DPG-PEG, | | | | | | |
| TRX-2 | | | | | | |
| DPG-PEG; | | | | | | |
| EXP22001910- | | | | | | |
| NL | | | | | | |
| Lipid 15, | 91.55 | 97.65 | 114.45 | 0.07 | 0.06 | 0.14 |
| DPG-PEG, | | | | | | |
| TRX-2; | | | | | | |
| EXP22001910- | | | | | | |
| NP | | | | | | |

### Example 14. Physiochemical Properties (Pre- and Post-Insertion with ACD8 Fab Conjugate T8) of Lipids 3, 4, 33, and 34 αCD20 CAR RNA Lipid Nanoparticles (LNPs)

Physiochemical properties (pre- and post-insertion with αCD8 Fab conjugate T8) of Lipids 3, 4, 33, and 34 αCD20 CAR RNA Lipid nanoparticles (LNPs).

Lipids 3, 4, 33, and 34 LNPs encapsulating an exemplary αCD20 CAR RNA (custom made by TriLink Biotechnologies Inc.) were prepared and characterized using methods described in Examples 5 to 8 above. Measured LNP size, PDI, charge and RNA content values of Lipids 3, 4, 33, and 34 LNPs are summarized in Table 21, Table 22, and Table 23. As seen in FIG. 10A, the initial mixing step and subsequent buffer exchanges into HBS and then MBS resulted in LNP sizes ≤125 nm with Lipids 3 and 4, and ≤100 nm with Lipids 33 and 34. All LNPs exhibited moderate to high encapsulation efficiency (<15% Dye accessible RNA) and >70% RNA recovery, with Lipids 33 and 34 trending better in terms of recovery and lower in terms of dye accessible RNA (FIG. 10D). The subsequent buffer exchange into pH 6.5 MES buffer and antibody insertion process (37° C., 4 hours) resulted in Lipid 3 and 4 LNP diameters in the 120 nm to 135 nm range, while Lipids 33 and 34 yield sub-100 nm diameters. Similarly, Lipid 3 and 4 LNP PDI trended slightly higher in the 0.15 to 0.21 range, while Lipids 33 and 34 yield PDI≤0.11, indicating slightly better size distribution properties. Lipid 4 and 34 LNPs showed moderate size increases after one freeze-thaw cycle, however, a notably larger size shifts were observed with lipid 3 and 33 LNPs. In summary, all four lipids tested resulted in viable CAR mRNA encapsulation and freeze-thaw stability as well as <150 nm final targeted LNP diameters. The ability of lipids 3, 4, 33, and 34 LNPs for inducing in vitro CAR protein expression in primary human T cells mediated by αCD3 or αCD8 T cell receptor targeting was evaluated as described in Examples 16 and 17 below.

TABLE 21
Lipids 3, 4, 33, 34 LNPs Size, Polydispersity (DLS) data in pH 7.4 HBS, pH 6.5
MBS and post aCD3 antibody Fab (hSP34) conjugate insertion.

| | | | Z-Avg. | Z-Avg. | | | Poly- | |
|---|---|---|---|---|---|---|---|---|
| | Z-Avg. | Z-Avg. | Size (nm); | Size | Poly- | Poly- | dispersity | Poly- |
| Ionizable | Size | Size | post- | (nm); | dispersity | dispersity | (DLS); Post- | dispersity |
| lipid, LNP | (nm); | (nm); | insertion; | Post | (DLS); | (DLS); | insertion; | (DLS); |
| no. | HBS | MBS | MBS | F/T | HBS | MBS | MBS | Post F/T |
| Lipid 3, | 108.6 | 121.5 | 131.8 | 135.5 | 0.19 | 0.16 | 0.15 | 0.17 |
| DPG-PEG; | | | | | | | | |
| EXP21004781- | | | | | | | | |
| N3 | | | | | | | | |
| Lipid 4, | 92.8 | 110.2 | 121.7 | 127.1 | 0.15 | 0.17 | 0.16 | 0.21 |
| DPG-PEG; | | | | | | | | |
| EXP21004781- | | | | | | | | |
| N5 | | | | | | | | |
| Lipid 33, | 92.2 | 94.1 | 97.5 | 116.9 | 0.21 | 0.1 | 0.10 | 0.18 |
| DPG-PEG; | | | | | | | | |
| EXP21004781- | | | | | | | | |
| N6 | | | | | | | | |
| Lipid 34, | 86.3 | 87.8 | 93.4 | 103 | 0.18 | 0.09 | 0.11 | 0.14 |
| DPG-PEG; | | | | | | | | |
| EXP21004781- | | | | | | | | |
| N4 | | | | | | | | |

TABLE 22
Lipids 3, 4, 33, 34 LNP charge, Zeta Potential, ZP (DLS, mV) in pH
5.5 MES and pH 7.4 HBS.

| | Charge (ZP, mV); | Charge (ZP, |
|---|---|---|
| Ionizable lipid, LNP no. | pH 5.5 | mV); pH 7.4 |
| Lipid 3, DPG-PEG; EXP21004781-N3 | 15.7 | 0.786 |
| Lipid 4, DPG-PEG; EXP21004781-N5 | 11.1 | −1.23 |
| Lipid 33, DPG-PEG; EXP21004781-N6 | 17.4 | 1.01 |
| Lipid 34, DPG-PEG; EXP21004781-N4 | 19.6 | 1.5 |

TABLE 23
Lipids 3, 4, 33, 34 LNP Dye Accessible RNA and total RNA content.

| | Nominal | | | | |
|---|---|---|---|---|---|
| | mRNA | Measured | Dye-accessible | | Dye- |
| Ionizable lipid, | Conc. | Total mRNA | mRNA | Total mRNA | accessible |
| LNP no. | (μg/mL) | (μg/mL) | (μg/mL) | Recovery (%) | mRNA (%) |
| Lipid 3, DPG-PEG; | 150 | 107.1 | 15.5 | 71.4 | 14.5 |
| EXP21004781-N3 | | | | | |
| Lipid 4, DPG-PEG; | 150 | 110.7 | 14.4 | 73.8 | 13.0 |
| EXP21004781-N5 | | | | | |
| Lipid 33, DPG-PEG; | 150 | 116.4 | 12.8 | 77.6 | 11.0 |
| EXP21004781-N6 | | | | | |
| Lipid 34, DPG-PEG; | 150 | 119.9 | 8.2 | 79.9 | 6.8 |
| EXP21004781-N4 | | | | | |

### Example 15. Method for Freezing (and Thaw) Process for LNP Suspension and LNP Characterization Post Freeze-Thaw

LNP suspension was mixed with a solution of 49 wt % sucrose solution in water and additional storage buffer (if needed) to achieve a final sample containing LNPs at approximately 45 μg/mL and sucrose at approximately 9.6 wt %. Aliquots of approximately 0.05 mL in 1.5 mL centrifuge tubes were then prepared from the final LNP sample containing sucrose. The aliquots were then placed in a −80° C. freezer for at least 2 h to freeze the samples. After freezing, an aliquot was thawed by placing it at room temperature for at least 10 min. The aliquot was then mixed by vortexing at 2500 rpm for approximately 5s. The thawed material was then analyzed for size by DLS as described in Example 3 above.

### Example 16. Method for the Primary Human T Cell Transfection with DiI-C18-5DS Labelled LNPs

CD3+ T cells were isolated from frozen peripheral blood mononuclear cells using an EasySep Human T Cell Isolation Kit on a RoboSep automated cell isolation system from STEMCELL. T cells were plated into a round bottom 96-well plate in RPMI cell culture media supplemented with glutamax, 10% fetal bovine serum, pen-strep, and 40 ng/ml IL-2. 100 μL of cell suspension was seeded per well at a density of 1M T cells/mL (100K T cells/well). Cells were allowed to rest for two hours in a 37° C. incubator, and then were transfected by gently adding 10 μL of a 22 μg/mL (by mRNA) nanoparticle suspension, resulting in a final mRNA concentration of 2 μg/mL (unless otherwise noted). Cells were gently mixed with a pipette and then incubated for 24 hours in a 37° C. incubator. After incubation the cells were diluted with FACS buffer (BD 554657) and analyzed using a BD Fortessa flow cytometer. Data were analyzed using FlowJo software from BD biosciences.

### Example 17. CD4 and CD69 Staining Protocol Post LNP Transfection

After 24 hours, cells were transferred to a 96-well conical bottom polypropylene plate and centrifuged at 350×g for 5 minutes. Supernatants were removed and transferred to a fresh conical bottom polypropylene plate for further analysis. Cells were washed by adding 200 μL FACS buffer (BD 554657), centrifuging at 350×g for 5 min, and then aspirating the supernatant from each well. BV421 anti-human CD69 (BioLegend 310930 clone FN50) and BV711 anti-human CD4 (BioLegend 344648 clone SK3) antibodies were diluted 100× by adding 100 μL of each antibody to 10 mL FACS buffer. 100 μL of the diluted antibody solution was added to each well and the plate was incubated at room temperature for 20 minutes. The plates were then washed by centrifuging at 350×g for 5 min, removing the supernatant, re-suspending in 200 μL FACS buffer, centrifuging at 350×g for 5 min and aspirating the supernatant from each well. Following the wash, cells were resuspended in 100 μL of 1.6% formaldehyde and stored at 4° C. until FACS analysis. FACS analysis was performed using a BD Fortessa equipped with a High Throughput Sampler.

### Example 18. Human IFN-Γ ELISA Method

IFN-γ was assayed using an R&D Duoset IL-2 ELISA kit, PN DY285B. Briefly: an Immulon 2HB 96-well plate (Thermo X1506319) was coated by adding 100 μL of a 2 μg/mL solution of the R&D IL-2 capture antibody to each well and then incubating the plate overnight at 4° C. The plate was washed three times with wash buffer (0.05 TWEEN-20 in pH 7.4 TRIS buffered saline, Thermo 28360), blocked with reagent diluent (0.1% BSA in wash buffer) for one hour at room temperature, and then washed an additional three times with wash buffer. Supernatants were diluted three-fold in reagent diluent and then 100 μL of diluted supernatant was added to each well. IFN-γ standards were prepared on the same plate by serial dilution. Plates were incubated for two hours at room temperature, washed three times with wash buffer. 100 μL of detection antibody diluted in reagent diluent was added, incubated for 2 hours at room temperature, and then the plate was washed three times with wash buffer. 100 μL of Streptavidin-HRP was added, incubated for 20 minutes at room temperature, and then the plate was washed three times with wash buffer. 100 μL of substrate solution (Thermo N301) was added, incubated for 20 minutes at room temperature and then the reaction was quenched by adding 100 μL of stop solution (Invitrogen SS04). Optical density at 450 nm was read on a SpectraMax M5 plate reader. IFN-γ concentration was quantified relative to a standard curve based on contemporaneously analyzed IFN-γ standards.

### Example 19. Anti-CD20 CAR (M1) Staining Protocol

Cells were transferred to a 96-well conical bottom plate and washed by centrifuging at 350×g for 5 minutes followed by resuspension in FACS buffer (BD 554657). An exemplary CAR protein (e.g., anti-CD20 CAR protein), which contains a FLAG tag, was detected by staining with M1 anti-Flag antibody (Sigma Aldrich F3040) conjugated to Alexa Fluor 488 (Invitrogen A3750) at room temperature for 20 minutes. Following staining, cells were washed twice by centrifugation at 350×g for 5 minutes followed by resuspension in FACS buffer. Cells were analyzed by FACS using a BD Fortessa flow cytometer (BD Biosciences).

Synthesis and purification procedure for the M1 anti-Flag antibody—Alexa Fluor 488 conjugate: M1 anti-FLAG antibody was buffer exchanged into pH 8.3 sodium bicarbonate buffer using a Zeba 40K MWCO spin column. An 8 mg/mL solution of Alexa Fluor 488 TFP ester (Invitrogen A37570) in DMSO was then added to a final AB: dye mass ratio of 10:1 and the mixture was allowed to react for 1 hour with gentle shaking. Fluorophore conjugated M1 was purified and buffer exchanged into pH 7.4 PBS by passing the reaction mixture through two sequential Zeba spin columns. Protein content and degree of labeling were assessed by UV-Vis spectroscopy.

### Example 20. In Vitro LNP Car Primary Human T Cell Transfection and Raji (B Cell) Co-Culture for Assessment of T Cell Function

CD3+ or CD8+ T cells were isolated from human PBMCs by using EasySep Human CD8+ T cell isolation kit (catalog #17953) and EasySep Human T cell isolation kit (catalog #17951) according to manufacturer's instructions. Isolated T cells were rested overnight in T cell media (RPMI+Glutamax, Gibco, catalog #61870-036) with 10% heat inactivated fetal bovine serum (HI-FBS, Gibco, catalog #16140-071) supplemented with 100 IU human IL-2 (Miltenyi catalog #130-097-748) at a density of 500,000 cells/well in a 96 well plate. T cells were then transfected with LNPs at a mRNA concentration of 2 μg/ml. CAR expression was detected 18 h post LNP transfection by using flow cytometry. Before co-culture with Raji B cells, the transfected T cells were washed twice with T cell media. Raji B cells were stained with cell trace violet (CTV, Thermo Fisher, catalog #C₃₄₅₇₁) and co-cultured with different effector to target (E:T) ratios of T cells in a 96 well plate format and incubated at 37° C. for 72 hours. 72 hours post co-culture the cells were stained with live/dead stain (eBioscience Fixable viability dye eFluor 780, Invitrogen catalog #2290917) and analyzed by flow cytometry. Data was analyzed with FlowJo and Graph Pad prism.

### Example 21. In Vitro Experimental Protocol for Whole Blood Transfection

This Example describes the method used to transfect immune cells in whole blood using Fab targeted mRNA LNPs.

Venous blood from healthy volunteers was anti-coagulated in heparin tubes (BD Biosciences #367526) and seeded at 50 μL in a 96-well round-bottom plate. Transfection of whole blood was carried out simply by adding nanoparticles containing 5 μg/mL mRNA to the cells and co-culturing at 37° C. until the time of analysis. To assess transfection efficiency, cells were analyzed 24-hours post-transfection by flow cytometry. LNPs used (with and without post-inserted targets) at 2.5 μg/mL: RDM073.23. Cells obtained from human blood were analyzed by flow cytometry. Prior to the analysis of whole blood transfection efficiency, red blood cells were lysed twice with VersaLyse Lysing Solution (Beckman Coulter #A09777) for 10 minutes at room temperature. Primary antibodies applied in the flow cytometry analysis of whole blood included the following: CD4-FITC (1:200) (BD Biosciences #555346), CD19-BUV395 (1:400) (BD Biosciences #563551), CD56-BUV737 (1:400) (BD Biosciences #741842). Fixable Viability Dye eFluor780 (eBiosciences #65-0865-14) was used to assess viability for all samples. For flow analysis, 1×10⁵ cells were Fc-blocked (BD Biosciences #564219) for 5 minutes on ice, followed by labeling dead cells with fixable viability dye eFluor780 and surface staining for 30 minutes on ice with specific antibodies.

Compensation for each fluorochrome was performed in the multicolor flow panels using positive and negative compensation beads. Fluorescence minus one (FMO) samples and unstained controls were included to determine the level of background fluorescence and to set the gates for the negative cell populations versus the positive cell populations.

All samples were acquired on a BD LSRFortessa X-20 (BD Biosciences) running FACSDIVA software (Becton Dickinson). All data collected were analyzed using FlowJo 10.7.1 software and GraphPad Prism version 9.0.

### Example 22. Protocol for In Vivo T Cell Reprogramming (Using GFP Reporter Protein) in Human T Cell Engrafted NSG-Mice

The following is a standard procedure for in-vivo reprogramming of immune cells with DiI LNP expressing GFP.

### Mice Strains and Humanization

6-8 weeks old NSG male mice were engrafted with 10 million PBMC of qualified donor in sterile PBS by tail vein injection. Individual body weight was monitored twice a week and blood samples were collected at appropriate interval to evaluate human immune cells engraftment.

### Evaluation of Human T Cell Engraftment in the Immunodeficient Mice

50 μl blood was collected by tail vein bleed from each mouse. Red blood cells were lysed using Versalyse, RBC lysis solution following protocol as instructed by manufacturer (Beckman Coulter A09777). Cells were stained with hCD45 & hCD3 to determine the engraftment of human T cells. After 15 days of PBMC injection, mice had anywhere from 30-60% huCD45+. These humanized mice were evaluated for reprogramming of immune cells by LNPs expressing Dil dye and GFP.

### Reprogramming of Immune Cells

At time zero, mice (n=4 per group) were injected with GFP expressing DiI LNPs (by i.v. with appropriate buffer). At each time point, 24 or 48 h depending on the example mice treated with either LNPs or buffer were sacrificed. Terminal blood and tissues collection was performed to determine Dil and GFP expression in different organs and immune cells as below.

### Tissue and Blood Sample Collection.

At above specified timepoints, mice were anesthetized with CO₂ before sample collection. For blood collection, the chest was opened to expose the heart. Up to 300 μl blood was drawn from the left ventricle and dispensed into a K3EDTA mini collect tube (Greiner Bio-One). Then a new syringe was used to draw remaining blood from the heart as much as possible. All the immune organs; spleen, bone marrow was isolated along with liver and lung. Immune cells were isolated from spleen, via smearing and shredding it through syringe and cell suspension was filtered through 70 μM cell strainer and was washed with PBS. Bone marrow was flushed with needle to collect all the immune cells. A piece of liver and lung tissue was gently grinded with tissue homogenizer and the homogenized and cells were isolated using Miltenyi liver dissociation kit, (Miltenyi Biotec, Catalog #130-105-807) and lung dissociation kit (Miltenyi Biotec, Catalog #130-0950927) and instruction were followed according to the manufacturing instruction.

### Immunophenotyping Analysis

Immune cells from blood and all the above organs were processed with Versalyse, RBC lysis buffer as per manufacturing instructions. Immune cells were stained with live/dead fixable dye and surface markers with standard flow analysis protocol as shown in Table 24. BD symphony flow cytometer was used to determine positive population.

TABLE 24
Staining antibodies for immunophenotyping analysis.

| Antigen | Fluorophore | Clone | Company | Catalog# |
|---|---|---|---|---|
| DiI | APC | NA | NA | NA |
| GFP | mRNA | NA | NA | NA |
| Anti-CD45 | BUV395 | HI30 | BD Biosciences | 563792 |
| Anti-CD3 | BUV805 | UCHT1 | BD Biosciences | 612895 |
| Anti-CD4 | BV711 | SK3 | BioLegend | 344648 |
| Anti-CD8 | BV421 | RPA-TB | BD Biosciences | 562428 |
| Anti-CD45 | BB700 | 30-F11 | BD Biosciences | 566439 |
| Anti-CD11b | BV785 | M1/70 | BioLegend | 101243 |
| Anti-F4/80 | PE Dazzle | BM8 | BioLegend | 123146 |
| Anti-CD31 | BUV737 | MEC 13.3 | BD Biosciences | 612802 |
| TruStain Monocyte | NA | NA | BioLegend | 462103 |
| Blocker | | | | |
| Arc Amine Comp | NA | NA | NA | 01-3333- |
| beads | | | | 42 |
| UltraComp eBeads | NA | NA | Invitrogen | 01-3333- |
| | | | | 42 |
| LIVE/DEAD Far | NA | NA | Invitrogen | L34974 |
| Red Stain | | | | |
| TruStain Fc X | NA | NA | Invitrogen | 422302 |

### Example 23. In Vitro Protein Expression (GFP) in Primary Human T Cells of Lipids 3, 6, 7 and Comparator Lipid ACD3 Targeted LNPS (DLIN-MC3-DMA and ALC-0315) Stored at 4° C. And after One Freeze-Thaw Cycle (Post −80° C. Storage)

This example compares the GFP protein expression resulting from LNP's derived from Lipids 3, 6 and 7 to LNPs made using comparator lipids DLin-MC3-DMA and ALC-0315. Nanoparticles bearing GFP encoding mRNA (and optionally a fluorescent dye label (Dil-C18-5DS)) were produced using the microfluidic mixing and buffer exchange processes described in Example 2 above. An αCD3 Fab-conjugate was incorporated into the parent LNPs to obtain the final Antibody targeted LNP formulation using the process described in Example 5 above. Particles thus produced were tested in vitro in primary human CD3+ T cells to assess reported gene expression.

Lipid 3 and ALC-0315 LNPs resulted in similar GFP protein expression levels as indicated by similar % GFP+ T cells and similar Mean Fluorescence Intensity (MFI) of the transfected T cells (see FIG. 11A, FIG. 11B (% GFP+), and FIG. 11C and FIG. 11D (GFP MFI)). This suggests that the antibody driven uptake was equally effective with both ionizable lipids and similar levels of ionizable lipid driven endosomal escape efficiencies were achieved. Lipid 7 and DLin-MC3-DMA lipids resulted in similar and lowest levels of GFP protein expression. Lipid 6 LNPs exhibited an intermediate level of GFP protein expression. The protein expression levels suggest that 9 carbon N-acyl substituent (lipid 3) enhances performance over an 11 carbon N-acyl substituent (of Lipids 6 and 7). The performance levels and relative order of LNP efficiency was preserved in all lipid nanoparticle formulation tested after 1 freeze/thaw cycle (Post −80° C. storage) as illustrated by comparison of % GFP+ cells and GFP MFI values before and after-80° C. storage (FIG. 11A versus FIG. 11B, and FIG. 11C versus FIG. 11D). While all lipid formulation tested were well tolerated by primary human T cells (FIG. 11E); some dose dependent loss in T cell viability was observed with lipids 7 and DLin-MC3-DMA.

### Example 24. In Vitro Protein Expression (GFP) in Primary Human T Cells of Lipids 3, 4 and Comparator Lipid ACD3 Targeted LNPs (DLIN-KC2-DMA and SM-102) Stored at 4° C. And after 1 Freeze-Thaw Cycle (Post −80° C. Storage)

This example compares the GFP protein expression resulting from LNP's derived from Lipids 3 and 4 to LNPs made using comparator lipids DLin-KC2-DMA and SM-102. Nanoparticles bearing GFP encoding mRNA (and optionally a fluorescent dye label (DiI-C18-5DS)) were produced using the microfluidic mixing and buffer exchange processes described in Example 2 above. An αCD3 Fab-conjugate was incorporated into the parent LNPs to obtain the final Antibody targeted LNP formulation using the process described in Example 5 above. Particles thus produced were tested in vitro in primary human CD3+ T cells to assess reported gene expression.

At ≥0.1 μg/mL dose, similar number of T cells were transfected with SM-102 and Lipid 4 LNPs, however, at doses below 0.1 μg/mL, a larger fraction of T cells were GFP+ when counted with SM-102 LNPs, and the GFP MFI values were consistently about 2-fold higher at all doses, suggesting more efficient cytosolic access/cytosolic release of encapsulated GFP mRNA relative to Lipid 4 LNPs. Lipid 3 and DLin-KC2-DMA LNPs performed similarly in terms of the fraction of T cells accessed as well as copies of GFP protein expressed on a per cell basis and were at levels by 2-fold lower relative to Lipid 4 LNPs. This trend indicates that the oleoyl tail groups of Lipid 4 resulted in more efficient cytosolic access/cytosolic release of encapsulated GFP mRNA relative to the linoleoyl tail groups of Lipid 3. The performance levels and relative order of LNP efficiency was well preserved in all lipid nanoparticle formulation tested after 1 freeze/thaw cycle (post −80° C. storage) as illustrated by comparison of % GFP+ cells and GFP MFI values before and after −80° C. storage (FIG. 12A versus FIG. 12B, and FIG. 12C versus FIG. 12D). Lipids 3, 4, and SM-102 LNPs were well tolerated by primary human T cells (FIG. 12E); however, a slightly greater loss in T cell viability was observed with DLin-KC2-DMA LNPs at the higher doses of 0.3 and 1 μg/mL.

### Example 25. In Vitro Protein Expression (GFP) in Primary Human T Cells of Lipids 1, 3, 8 and Comparator Lipid ACD3 Targeted LNPs (DLIN-KC2-DMA) Stored at 4° C. And after 1 Freeze-Thaw Cycle (Post −80° C. Storage)

This example compares the GFP protein expression resulting from LNP's derived from Lipids 1, 3 and 5 to LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2 above. An αCD3 Fab-conjugate was incorporated into the parent LNPs to obtain the final Antibody targeted LNP formulation using the process described in Example 5 above. Particles thus produced were tested in vitro in primary human CD3+ T cells to assess reported gene expression.

At all dose levels, Lipid 1 and DLin-KC2-DMA LNPs performed similarly both in terms of fraction of GFP+ T cells (reflected by % GFP+ cells) and the copies of GFP protein produced on a per cell basis (reflected by GFP MFI values). Lipid 3 LNPs resulted in a roughly 3-fold higher fraction of GFP+ T cells relative to Lipid 1 LNPs and the GFP MFI values were consistently about 3-fold higher at all doses suggesting more efficient cytosolic access/cytosolic release of encapsulated GFP mRNA with Lipid 3 LNPs relative to Lipid 1, indicating that the 9 carbon N-acyl substituent (of Lipid 3) is favorable for efficient cytosolic access/cytosolic release of encapsulated GFP mRNA relative to the 10 carbon N-acyl substituent (of Lipid 1). Highest GFP protein expression was observed with Lipid 5 LNPs as indicated by the higher % GFP+ and GFP MFI values at all dose levels. This indicates that further reduction in carbon number of the N-acyl substituent to 8 carbon skeleton further improves the mRNA delivery efficiency. However, as noted in Example 9 above, Lipid 5 LNPs exhibited larger size shifts in the buffer exchange and antibody conjugate insertion steps resulting in final LNP hydrodynamic diameters >150 nm indicating that 9-carbon N-acyl substitution skeleton proves optimal for LNP size (for enabling 0.2 micron sterile filtration) as well as the ability to induce reporter gene expression. The performance levels and relative order of LNP efficiency was well preserved in all formulation tested after 1 Freeze/Thaw cycle (Post −80° C. storage) as illustrated by comparison of % GFP+ cells and GFP MFI values before and after −80° C. storage (FIG. 13A versus FIG. 13B, and FIG. 13C versus FIG. 13D). Lipids 1, 3, 5 LNPs were well tolerated by primary human T cells (FIG. 13E).

### Example 26. In Vitro Protein Expression (GFP) in Primary Human T Cells of Lipids 1, 8 and Comparator Lipid ACD3 Targeted LNPs (DLIN-KC2-DMA) Stored at 4° C. And after 1 Freeze-Thaw Cycle (Post −80° C. Storage)

This example compares the GFP protein expression resulting from LNP's derived from Lipids 1 and 8. Nanoparticles bearing GFP encoding mRNA (and optionally a fluorescent dye label (DiI-C18-5DS)) were produced using the microfluidic mixing and buffer exchange processes described in Example 2 above. An αCD3 Fab conjugate was incorporated into the parent LNPs to obtain the final antibody targeted LNP formulation using the process described in Example 5 above. Particles thus produced were tested in vitro in primary human CD3+ T cells to assess reported gene expression.

At all dose levels, both Lipid 1 and LNPs resulted in similar fraction of GFP+ T cells (reflected by % GFP+ cells), however, Lipid 8 LNPs out-performed Lipid 1 LNPs by a factor of 3 in terms of copies of GFP protein produced on a per cell basis (reflected by GFP MFI values). This indicates that the β-ethyl octanoyl N-acyl substituent (of Lipid 8) improves cytosolic access/cytosolic release of encapsulated GFP mRNA relative to the α-ethyl octanoyl N-acyl substituent (of Lipid 1). The overall effect of the β-ethyl substitution is better LNP size distribution properties (relative to Lipid 1 as illustrated in Example 9) and gain in mRNA delivery efficiency resulting in a 2-fold improvement in reporter gene expression levels despite the 10 carbon N-acyl substitution pattern. This indicates that both LNP properties and delivery efficiency are tunable via size (carbon number) and geometry (β-ethyl octanoyl versus α-ethyl octanoyl) of the N-acyl substituent. The performance of Lipid 8 LNP's was well preserved after 1 Freeze Thaw cycle (Post −80° C. storage) as illustrated by comparison of % GFP+ cells and GFP MFI values before and after −80° C. storage (FIG. 14A and FIG. 14B). Both Lipids 1 and 8 LNPs were well tolerated by primary human T cells (FIG. 14C).

### Example 27. In Vitro Protein Expression (GFP) in Primary Human T Cells of Lipids 8, 9, 10 and Comparator Lipid ACD3 Targeted LNPs (DLIN-KC3-DMA) Stored at 4° C. And after 1 Freeze-Thaw Cycle (Post −80° C. Storage)

This example compares the GFP protein expression resulting from LNP's derived from Lipids 8, 9, 10 and comparator lipid DLin-KC2-DMA LNPs. Nanoparticles bearing GFP encoding mRNA (and optionally a fluorescent dye label (DiI-C18-5DS)) were produced using the microfluidic mixing and buffer exchange processes described in Example 2 above. An αCD3 Fab conjugate was incorporated into the parent LNPs to obtain the final Antibody targeted LNP formulation using the process described in Example 5 above. Particles thus produced were tested in vitro in primary human CD3+ T cells to assess reported gene expression.

As shown in FIGS. 15A and 15C, at all dose levels, Lipid 8 and 9 LNPs performed similarly both in terms of fraction of GFP+ T cells (reflected by % GFP+ cells) and the copies of GFP protein produced on a per cell basis (reflected by GFP MFI values). Thus, the succinic acid derived 14 carbon N-acyl substituent (in Lipid 9) results in similar efficiency of cytosolic access/cytosolic release of the mRNA payload relative to the 10 carbon N-acyl substituent (of Lipid 8) indicating that introduction of a carboxylate ester (and added polarity of the O-atoms) in N-acyl substituent balances and counters the loss of lipid efficiency seen with Lipids 6 and 7 (that both feature a 11 carbon N-acyl substituent). As illustrated in Example 10 above, Lipid 9 LNPs exhibit size distribution properties comparable to those of Lipids 6, 7 and 8 suggesting that biodegradable ester linkages (as in Lipid 9) can be introduced into the N-acyl substituent without loss of LNP size distribution properties. Lipid 10 LNPs out-performed Lipid 9 LNPs both in terms of fraction of GFP+ T cells (reflected by % GFP+ cells) and the copies of GFP protein produced on a per cell basis (reflected by GFP MFI values) by a factor of 1.5. Suggesting that reduction in the total carbon count to 12 (in lipid 10) versus 14 (in Lipid 9) further improved lipid efficiency. Thus, analogous to trends observed with Lipids 3, 5, 6, 7 and 8 LNPs, the activity of succinic acid derived biodegradable N-acyl substituents in Lipids 9 and 10 is also tunable. The performance of Lipid 10 LNPs was well preserved after 1 Freeze/Thaw cycle (Post −80° C. storage) as illustrated by comparison of % GFP+ cells and GFP MFI values before and after −80° C. storage (FIG. 15A versus FIG. 15B, and FIG. 15C versus FIG. 15D). Lipids 9 and 10 LNPs were well tolerated by primary human T cells (FIG. 15E and FIG. 15F).

### Example 28. In Vitro Protein Expression (GFP) in Primary Human T Cells of Lipids 3, 4, 9, 15 and Comparator Lipid (DLIN-KC2-DMA) ACD3 Targeted LNPS Stored at 4° C. And after 1 Freeze-Thaw Cycle (Post −80° C. Storage)

This example compares the GFP protein expression resulting from LNP's derived from Lipids 3, 4, 9, 15 and LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2 above. An αCD3 Fab conjugate was incorporated into the parent LNPs to obtain the final antibody targeted LNP formulation using the process described in Example 5 above. Particles thus produced were tested in vitro in primary human CD3+ T cells to assess reported gene expression.

At all dose levels, performance improvements (both in terms of fraction of GFP+ T cells as reflected by % GFP+ cells and the copies of GFP protein produced on a per cell basis as reflected by GFP MFI values) were observed between lipid 3 versus lipid 4 and between lipid 9 versus lipid 15 indicating that the oleoyl tail groups (in Lipids 4 and 15) result in improved performance over the corresponding linoleoyl tail groups (in Lipids 3 and 9). Thus, gains in lipid oxidative stability via lower lipid tail unsaturation (with singly unsaturated oleic acid) without loss of LNP efficiency typically observed with the lower lipid membrane fluidity. Furthermore, in contrast to Lipid 4 where poorer LNP size distribution properties were observed (relative to Lipid 3), Lipid 15 LNPs exhibited size distribution properties similar to those observed with Lipid 9 LNPs indicating that the N-acyl substituent plays a more significant role in determining the LNPs size distribution properties compared to the role of the lipid tail groups. The performance levels and relative order of LNP efficiency was well preserved in all formulation tested after 1 Freeze Thaw cycle (Post −80° C. storage) as illustrated by comparison of % GFP+ cells and GFP MFI values before and after −80° C. storage (FIG. 16A versus FIG. 16B, and FIG. 16C versus FIG. 16D). Lipids 3, 4, 9, and 15 LNPs were well tolerated by primary human T cells (FIG. 16E).

### Example 29. In Vitro Protein Expression (GFP) in Primary Human T Cells of Lipids 3, 4, 9, 15 ACD8 (TRX-2) Targeted LNPS and the Corresponding Non-Targeted Parent LNPS (Both Stored at 4° C.)

This example compares the GFP protein expression resulting from LNP's derived from Lipids 3, 4, 9, 15 and the corresponding non-targeted parent LNPs. Nanoparticles bearing GFP encoding mRNA (and optionally a fluorescent dye label (DiI-C18-5DS)) were produced using the microfluidic mixing and buffer exchange processes described in Example 2 above. An αCD8 Fab-conjugate, TRX2, were incorporated into the parent LNPs to obtain the final Antibody targeted LNP formulation using the process described in Example 5 above. Particles thus produced were tested in vitro in primary human CD8 T cells to assess reported gene expression. Additionally, parent LNPs (without any targeting Fab conjugate incorporated into the LNP corona) were tested to check any non-specific uptake in the CD8+ T cell population.

As seen in FIG. 17A and FIG. 17B, Lipids 9 and 15 resulted in similar levels of GFP protein expression the CD8+ T cell population (both in terms of fraction of GFP+ T cells as reflected by % GFP+ cells and the copies of GFP protein produced on a per cell basis as reflected by GFP MFI values). FIG. 17C and FIG. 17D, show the % DiI+ (dye) T cells and the DiI MFI reflective of the relative levels of Dil dye labeled LNPs taken up by the CD8+ T cell population. Notably, similar % DiI+ T cells were observed in all targeted formulations while buffer control-like dye levels (Dil MFI values, FIG. 17D) were seen in parent (non-targeted) formulations confirming the role of the TRX2 targeting Fab in association and uptake into CD8+ T cells. As expected, no GFP protein expression was observed in the non-targeted parent LNP formulations indicating lipid chemistry did not play a role this TRX2 mediated cellular uptake mechanism. Lipids 3, 4, 9, and 15 αCD8 (TRX2) targeted LNPs were well tolerated by primary human T cells (FIG. 17E) with cell viability trending a measurable lower in Lipid 9 and Lipid 15 formulations possibly due to the higher levels of GFP protein expression observed with these lipids (as indicated by the higher GFP MFI values seen FIG. 17B).

### Example 30. In Vitro Protein Expression (GFP) in Primary Human T Cells of Lipids 3, 4, 9, 15 ACD8 (T8) Targeted LNPS and the Corresponding Non-Targeted Parent LNPS (Both Stored at 4° C.)

This example compares the GFP protein expression resulting from LNP's derived from Lipids 3, 4, 9, 15 and the corresponding non-targeted parent LNPs (both after one Freeze/Thaw cycle). Nanoparticles bearing GFP encoding mRNA (and optionally a fluorescent dye label (DiI-C18-5DS)) were produced using the microfluidic mixing and buffer exchange processes described in Example 2 above. An αCD8 Fab conjugate, T8, was incorporated into the parent LNPs to obtain the final Antibody targeted LNP formulation using the process described in Example 5 above. Particles thus produced were tested in vitro in primary human CD8 T cells to assess reported gene expression. Additionally, parent LNPs (without any targeting Fab conjugate incorporated into the LNP corona) were tested to check any non-specific uptake in the CD8+ T cell population.

As seen in FIG. 18A and FIG. 18B, with this T8 αCD8 targeting strategy, Lipid 15 LNPs out-performed Lipid 9 LNPs with 2- to 3-fold higher reporter gene expression observed (both in terms of fraction of GFP+ T cells as reflected by % GFP+ cells and the copies of GFP protein produced on a per cell basis as reflected by GFP MFI values). FIG. 18C and FIG. 18D, show the % DiI+ (dye) T cells and the Dil MFI reflective of the relative levels Dil dye labelled LNPs taken up by the CD8+ T cell population. Notably, similar % DiI+ T cells were observed in all targeted formulations while buffer control-like dye levels (Dil MFI values, FIG. 18D) were seen in parent (non-targeted) formulations confirming the role of the T8 targeting Fab in association and uptake into CD8 T cells. As expected, no GFP protein expression was observed with the non-targeted parent LNP formulations indicating lipid chemistry did not play a role this T8 mediated cellular uptake mechanism. Lipids 3, 4, 9, and 15 αCD8 (T8) targeted LNPs were well tolerated by primary human T cells (FIG. 18E) with cell viability trending a measurable lower in Lipid 9 and Lipid 15 formulations possibly due to the higher levels of GFP protein expression observed with these lipids (as indicated by the higher GFP MFI values seen FIG. 18B).

### Example 31. In Vitro Protein Expression (GFP) in Primary Human T Cells of Lipids 2, 3, 31 and 32 and Comparator Lipid (DLIN-KC2-DMA) ACD3 (HSP34) Targeted LNPs Stored at 4° C.

This example compares the GFP protein expression resulting from LNP's derived from Lipids 2, 3, 31 and 32 to LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2 above. An αCD3 Fab conjugate was incorporated into the parent LNPs to obtain the final antibody targeted LNP formulation using the process described in Example 5 above. Particles thus produced were tested in vitro in primary human CD3+ T cells to assess reported gene expression.

At all dose levels, Lipid 2 and 3 out-performed Lipid 31, 32 and DLin-KC2-DMA LNPs both in terms of fraction of GFP+ T cells (reflected by % GFP+ cells) and the copies of GFP protein produced on a per cell basis (reflected by GFP MFI values) (see FIG. 19A and FIG. 19B). This is consistent with higher apparent pKa of Lipid 31 and Lipid 32 LNPs relative to Lipid 2 and 3 LNPs and less pronounced change in LNP charge state under acidic endosomal pH conditions (as described in Example 13 above) and consequently lower levels of cytosolic access expected with Lipids 31 and 32. Lipids 2, 3, 31 and 32 LNPs were well tolerated by primary human T cells (FIG. 19C).

### Example 32. In Vitro Protein Expression (GFP) in Primary Human T Cells of Lipids 3, 33 and 34 and Comparator Lipid (DLIN-KC2-DMA) ACD3 Targeted LNPs Stored At 4° C. And after 1 Freeze-Thaw Cycle (Post −80° C. Storage) and Non-Binding (Mutated OKT8) Antibody Targeted LNPs

This example compares the GFP protein expression resulting from LNP's derived from Lipids 3, 31, 32 and LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2 above. An αCD3 Fab conjugate was incorporated into the parent LNPs to obtain the final antibody targeted LNP formulation using the process described in Example 5 above. Additionally, mock targeted LNPs were produced by incorporation of a non-binding mutated antibody Fab (mut-OKT8) conjugate into the parent LNPs using the antibody conjugate insertion method described in Example 4 above. Particles thus produced were tested in vitro in primary human CD3+ T cells to assess reported gene expression.

At all dose levels, Lipid 3 and 33 out-performed Lipid 34 and DLin-KC2-DMA LNPs both in terms of fraction of GFP+ T cells (reflected by % GFP+ cells) and the copies of GFP protein produced on a per cell basis (reflected by GFP MFI values). Furthermore, mut-OKT8 functional Lipid 33 and Lipid 34 LNPs did not result in any protein expression confirming the role of the αCD3 Antibody (hSP34) in the cellular uptake mechanism observed with these lipid formulations. The performance levels and relative order of LNP efficiency was well preserved in all formulations tested after 1 Freeze/Thaw cycle (Post −80° C. storage) as illustrated by comparison of % GFP+ cells and GFP MFI values before and after −80° C. storage (see FIG. 20A versus FIG. 20B, and FIG. 20C versus FIG. 20D). Lipids 3, 33 and 34 LNPs were well tolerated by primary human T cells (FIG. 20E).

### Example 33. In Vitro Car Protein Expression (M1 Tagged Extracellular Domain) In Primary Human T-Cells of Lipids 3, 4, 9 and 34, ACD3 Targeted ACD20 CAR-RNA LNPs Stored at 4° C. And after 1 Freeze-Thaw Cycle (Post −80° C. Storage)

This example compares the αCD20 CAR protein expression levels resulting from LNP's derived from Lipids 3, 4, 9 and 33. Nanoparticles bearing an exemplary αCD20 CAR encoding mRNA were produced using the microfluidic mixing and buffer exchange processes described in Example 2 above. An αCD3 Fab conjugate (hSP34) was incorporated into the parent LNPs to obtain the final antibody targeted LNP formulation using the process described in Example 5 above. Particles thus produced were tested in vitro in primary human CD3+ T cells to assess CAR expression via detection of an M1 tag on the extracellular domain of the TTR-023 transmembrane CAR protein.

At all dose levels tested, higher CAR levels were detected with lipids 3 and 4 relative to lipids 9 and 33 with Lipid 4 performing the best in this αCD3 targeting pathway. The relative levels of CAR expression are consistent with oleoyl tail group of Lipid 4 resulting in improved performance over the corresponding linoleoyl tail group of Lipids 3 and 9 seen with reported (GFP) gene expression in Examples 28, 29, and 30 above. Furthermore, this illustrates that this relative order of lipid efficiency is preserved between a reporter gene (GFP) and a therapeutic cargo (αCD20 CAR protein) in this αCD3 mediated targeting and cellular uptake mechanism. The performance levels and relative order of LNP efficiency was well preserved in Lipid 3, 4 and 9 LNPs after 1 Freeze/Thaw cycle (Post −80° C. storage). In contrast, a drop in CAR expression was detected at all doses of Lipid 33 LNP as illustrated by comparison of % M1+ cells and M1 MFI values before (4° C. stored) and after −80° C. storage (see FIG. 21A verses FIG. 21B, and FIG. 21C versus FIG. 21D). Lipids 3, 4, 9, and 33 LNPs were well-tolerated by primary human T-cells (FIG. 21E and FIG. 21F).

### Example 34. In Vitro Car Protein Expression (M1 Tagged Extracellular Domain) In Primary Human T-Cells of Lipids 3, 4, 9 and 34, ACD8 (T8) Targeted ACD20 CAR-RNA LNPS Stored at 4° C.

This example compares the αCD20 CAR protein expression levels resulting from LNP's derived from Lipids 3, 4, 9 and 33. Nanoparticles bearing an exemplary αCD20 CAR encoding mRNA were produced using the microfluidic mixing and buffer exchange processes described in Example 2 above. An αCD8 Fab conjugate (T8) was incorporated into the parent LNPs to obtain the final antibody targeted LNP formulation using the process described in Example 5 above. Particles thus produced were tested in vitro in primary human CD3+ T cells to assess CAR expression via detection of an M1 tag on the extracellular domain of the CD20 transmembrane CAR protein. Transfected cells were gated as CD4+ (CD4 population) and CD4-(CD8 population) and CAR expression was monitored (as % M1+ and M1 MFI) in the two populations to assess specificity in the CD8 population with the αCD8 (T8) targeting strategy.

At all dose levels tested, higher CAR levels in the CD4-population (CD8 cells, as illustrated by the M1% and M1 MFI values in FIG. 22A and FIG. 22B) were detected with lipids 4 and 9 relative to lipids 3 and 33 with Lipids 4 and 9 performing equally well in this CD8 targeting pathway. CAR levels similar to buffer control (PBS) were detected in the CD4+ population (CD4 cells, as illustrated by the M1% and M1 MFI values in FIG. 22C and FIG. 22D) confirming that a T8 antibody enables receptor mediated uptake specifically into CD8 T-cells. Lipids 3, 4, 9, and 33 LNPs were well tolerated by primary human T-cells (FIG. 22E).

### Example 35. In Vitro Car Protein Expression (M1 Tagged Extracellular Domain) In Primary Human T-Cells of Lipids 3, 4, 9 and 34, ACD8 (T8) Targeted ACD20 CAR-RNA LNPS After 1 Freeze-Thaw Cycle (Post −80° C. Storage)

This example compares the αCD20 CAR protein expression levels resulting from LNP's derived from Lipids 3, 4, 9 and 33 after 1 Freeze/Thaw cycle (post −80° C. storage). Nanoparticles bearing an exemplary αCD20 CAR encoding mRNA were produced using the microfluidic mixing and buffer exchange processes described in Example 2 above. An αCD8 Fab conjugate (T8) was incorporated into the parent LNPs to obtain the final antibody targeted LNP formulation using the process described in Example 5 above. Particles thus produced were subjected to 1 Freeze/Thaw cycle and then tested in vitro in primary human CD3+ T cells to assess CAR expression via detection of an M1 tag on the extracellular domain of the αCD20 transmembrane CAR protein. Transfected T-cells were gated as CD4+ (CD4 population) and CD4− (CD8 population) and CAR expression was monitored (as % M1+ and M1 MFI) in the two populations to assess specificity in the CD8 population with the &CD8 (T8) targeting strategy.

At all dose levels tested, higher CAR levels in the CD4-population (CD8 cells, as illustrated by the M1% and M1 MFI values in FIG. 23A and FIG. 23B) were detected with lipids 4 and 9 relative to lipids 3 and 33 while Lipids 4 and 9 performed equally well in this CD8 targeting pathway. CAR levels similar to buffer control (PBS) were detected in the CD4+ population (CD4 cells, as illustrated by the M1% and M1 MFI values in FIG. 23C and FIG. 23D) confirming that a T8 antibody enables receptor mediated uptake specifically into CD8 T-cells. Both levels of CAR expression and specificity to CD8 T-cells were observed with particles that had been subjected to 1 Freeze/Thaw cycle confirming that the integrity and function of the formulations were preserved. Lipids 3, 4, 9, and 33 LNPs were well tolerated by primary human T-cells (FIG. 23E).

### Example 36. GFP Protein Expression and LNP Association (Measured as DiI-Dye Fluorescence) in CD8 and CD4 T Cell with Lipid 9, 15, DLIN-KC3-DMA Lipid ACD3 (HSP34) Targeted LNPS in Human Whole Blood

Lipid 9, 15 and DLin-KC3-DMA αCD3 and αCD8 targeted (and a non-binding antibody, mutOKT8, as a negative control) GFP mRNA LNPs were dosed to human venous whole blood, incubated for 24 hours and analyzed using the protocol described in Example 21 above for whole blood transfections.

As seen in FIGS. 24A-24D, αCD3 (hSP34) targeted LNPs resulted in GFP expression in both CD4+ and CD8+ T cells while αCD8 (TRX2) targeted LNPs resulted in GFP expression selective in CD8 T cells only as expected. Furthermore, non-binding (mutOKT8) control LNP resulted in no GFP expression in either cell type. Lipid 9 and 15 αCD3 (hSP34) targeted LNPs exhibited similar levels of GFP expression indicating that the two lipids are equally efficient in this cellular uptake pathway. However, in the CD8 binding and uptake pathway, αCD8 (TRX2) targeted Lipid 15 LNPs outperformed the corresponding Lipid 9 LNPs transfecting both a larger fraction of CD8+ T cells (expressed as % GFP+ cells) as well as more copies of GFP protein on a per cell basis (expressed as GFP MFI).

As seen in FIGS. 24E-24H, αCD3 (hSP34) targeted LNPs resulted in binding to both CD4+ and CD8+ T cells while αCD8 (TRX2) targeted LNPs bound selectively to CD8+ T cells only (measure as Dil dye % and MFI in the two cell populations). Furthermore, non-binding (mutOKT8) control LNP resulted in no significant binding to either T cell type as expected. Lipid 9 and 15 LNPs bound to similar fractions of CD4+ and CD8+ T cells as indicated by similar % DiI+ cells in both populations. However slightly higher binding was observed with Lipid 15 LNPs in the CD8 binding and uptake pathway relative to Lipid 9 LNPs as indicated by brighter dye fluorescence intensity (DiI MFI values) on a per cell basis.

### Example 37. GFP Protein Expression in NK Cells, Granulocytes, and B Cells, with Lipid 9, 15, DLIN-KC3-DMA Lipid αCD3 (Hsp34) Targeted LNPS in Human Whole Blood

Lipid 9, 15 and DLin-KC3-DMA αCD3 and αCD8 targeted (and a non-binding Antibody, mutOKT8, as a negative control) GFP mRNA LNP transfected whole blood samples (of Example 38 below) were also analyzed for GFP expression in NK cells, Granulocytes, and B cells using the protocol described in Example 21 above for whole blood transfections.

As seen in FIG. 25A and FIG. 25B, both αCD3 (hSP34) targeted LNPs and & CD8 (TRX2) targeted LNPs resulted in GFP expression in NK cells as expected. Furthermore, no GFP expression was observed in NK cells with the non-binding (mutOKT8) control LNP. Lipid 9 and 15 αCD3 (hSP34) targeted LNPs exhibited similar levels of GFP expression in NK cells indicating that the two lipids are equally efficient in this cellular uptake pathway. However, in the CD8 binding and uptake pathway, αCD8 (TRX2) targeted Lipid 15 LNPs outperformed the corresponding Lipid 9 LNPs transfecting both a larger fraction of NK cells (expressed as % GFP+ cells) as well as more copies of GFP protein on a per cell basis (expressed as GFP MFI). As seen in FIGS. 25C-25F, neither targeting modalities resulted in any significant GFP expression in Granulocytes and B cells.

### Example 38. LNP Association (Measured as DiI-Dye Fluorescence) in NK Cells, Granulocytes, and B Cells with Lipid 9, 15, DLIN-KC3-DMA Lipid αCD3 (Hsp34) Targeted LNPS in Human Whole Blood

Lipid 9, 15 and DLin-KC3-DMAαCD3 and αCD8 targeted (and a non-binding Antibody, mutOKT8, as a negative control) GFP mRNA LNP transfected whole blood samples (of Example 36 above) were also analyzed for binding to NK cells, Granulocytes, and B cells using the protocol described in Example 23 above for whole blood transfections.

As seen in FIG. 26A and FIG. 26B, both a CD3 (hSP34) targeted LNPs and a CD8 (TRX2) targeted LNPs bound to NK cells as expected. Furthermore, the non-binding (mutOKT8) control LNP did not result in any significant bind to NK cells as expected. As seen in FIGS. 26C-26F, both targeting modalities resulted in non-specific binding to Granulocytes and B cells however, no GFP expression was observed, indicating that RNA was not delivered to the cytosol of either cell type.

### Example 39. In Vitro Protein Expression (GFP) and LNP Association (Measured as DiI-Dye Fluorescence) in Primary Human T Cells of a CD3 Targeted Lipids 15, 9, 10, And 13 and Comparator (DLIN-KC3-DMA) LNPS Stored at 4° C. And after 1 Freeze-Thaw Cycle (Post −80° C. Storage)

This example compares the GFP protein expression resulting from LNP's derived from Lipids 15, 9, 10 and comparator lipid DLin-KC2-DMA LNPs. Nanoparticles bearing GFP encoding mRNA (and optionally a fluorescent dye label (DiI-C18-5DS)) were produced using the microfluidic mixing and buffer exchange processes described in Examples 2 and 6 above. An αCD3 Fab conjugate was incorporated into the parent LNPs to obtain the final Antibody targeted LNP formulation using the process described in Example 5 above. Particles thus produced were tested in vitro in primary human CD3+ T cells to assess reported gene expression.

As shown in FIG. 28A and FIG. 28B, at all dose levels, Lipid 15 and 10 LNPs performed similarly both in terms of fraction of GFP+ T cells (reflected by % GFP+ cells) and the copies of GFP protein produced on a per cell basis (reflected by GFP MFI values) and significant better relative to Lipid 9 LNPs with respect to the copies of GFP protein produced on a per cell basis (reflected by GFP MFI values) particularly at the lower dose levels. Thus, improvements over lipid 9 performance could be achieved either by modification of the O-acyl substituent (from Linoleoyl of Lipid 9 to Oleoyl of Lipid 15) or by modification of N-acyl substituent (from the succinic acid derived 14 carbon N-acyl substituent of Lipid 9 to the succinic acid derived 12 carbon N-acyl substituent of Lipid 10).

Additionally, as seen in FIG. 28C and FIG. 28D, Lipid 9 and Lipid 13 LNPs exhibited similar cell association levels (both in terms of fraction of DiI+ cells and in terms of the copies of cell associated LNPs reflected by Dil MFI values shown in FIG. 28C and FIG. 28D) at all dose levels, however, Lipid 9 outperformed Lipid 13 LNPs in terms of protein expression both in terms of fraction of GFP+ T cells (reflected by % GFP+ cells) and the copies of GFP protein produced on a per cell basis (reflected by GFP MFI values) suggesting inferior endosomal escape capabilities of Lipid 13 LNPs relative to Lipid 9 LNPs. The performance of Lipids 10 and 13 LNPs was well preserved after 1 Freeze/Thaw cycle (Post −80° C. storage) as illustrated by comparison of % GFP+ cells and GFP MFI values before (4° C. stored) and after-80° C. storage as seen in FIG. 28A and FIG. 28B.

### Example 40. In Vivo T Cell Reprogramming Using GFP Reporter Protein and A-CD8 (TRX-2) Targeted LNPS Based on Lipids 9, 15 and Comparator Lipid DLIN-KC3-DMA (and Formulated with 1.5 Mol % DPG-PEG) in Human T Cell Engrafted NSG Mice

NSG Mice were dosed using protocols described in Example 22 above. Plasma samples were drawn immediately prior to sacrificing the animals and spleen and liver harvested for analysis 24 hours post injection following the study design shown in Table 25.

TABLE 25
NSG Mice GFP T cell Reprogramming study design.

| | | Formulation | | Targeting |
|---|---|---|---|---|
| Groups | Mice # | Dosed | Payload | Antibody |
| 1 | 2 | Buffer Control | NA | NA |
| 2 | 4 | Lipid 15; 1.5% | GFP-mRNA | TRX-2 |
| | | DPG-PEG; DiI | | |
| | | dye labelled | | |
| 3 | 4 | DLin-KC3-DMA; | GFP-mRNA | TRX-2 |
| | | 1.5% DPG-PEG; | | |
| | | No dye label used | | |
| 4 | 3 | Lipid 9; 1.5% | GFP-mRNA | TRX-2 |
| | | DPG-PEG; DiI | | |
| | | dye labelled | | |

CD4+ and CD8+ T cells were stained sorted (by FACS) in blood, spleen and liver samples, liver samples were additionally stained and sorted for hepatocytes, endothelial cells, Kupffer cells, mouse macrophages and mouse myeloid cells using the Flow Panel depicted in Table 26 and Table 27. GFP fluorescence and DiI Dye fluorescence was used for quantitation of GFP protein expression (expressed as % GFP+ Cells and Mean Fluorescence Intensity (MFI) of GFP+ Cells) and LNP association (via Dil label fluorescence, expressed as % DiI+ cells and DiI-MFI of DiI+ Cells) in the stated cell types of interest.

TABLE 26
Cell Markers (Dead-Live, LNP, Protein, HuCD45,
huCD3, huCD4) and Fluorophores used.

| | Dead live | LNP | GFP- | | | |
|---|---|---|---|---|---|---|
| Markers | stain | (Dil dye) | Protein | huCD45 | huCD3 | huCD4 |
| Fluorophore | e-flour780 | APC | Green | BUV395 | BUV805 | BV711 |
| | | | Fluorescence | | | |

TABLE 27
Cell markers (Dead-Live, huCD8, muCD45, hu/muCD11b,
muCD31, F4/80) and Fluorophores used.

| Markers | Dead live stain | huCD8 | muCD45 | Hu/muCD11b+ | muCD31 | F4/80 |
|---|---|---|---|---|---|---|
| Fluorophore | e-flour780 | BV421 | BB700 | BV785 | BUV737 | PE Dazzle |

As seen in FIGS. 29A-29C, 7-25% GFP+ cells were detected in the CD8+ T cell population in blood, spleen and liver samples, while <3% of the CD4+ T cells were GFP+ confirming differential in vivo reprogramming of the CD8+ T cell population and LNP targeting using TRX-2 αCD8 antibody. As seen in FIGS. 30A-30C, LNP association was specific to the CD8+ T cell population in blood and spleen samples, however, significant levels of association to the CD4+ population as well as endothelial cells, Kupffer Cells, and mouse macrophages was observed in the liver samples. Notably, despite non-specific LNP association, no GFP protein was detected (FIG. 29C) indicating that off target LNP association does not result in off-target mRNA delivery and protein expression.

### Example 41. Preparation of LNPS by Microfluidic Mixing Using Exemplary Lipids

This Example describes the production of mRNA-loaded LNPs using exemplary materials and microfluidic mixing process.

LNPs encapsulating an mRNA payload were prepared by mixing an aqueous mRNA solution and an ethanolic lipid blend solution (containing the ionizable lipid Lipid 15, DSPC, DPG-PEG and Cholesterol at lipid ratios shown in Table 28) using an in-line microfluidic mixing process. The mRNA stock solution was diluted in pH 4 acetate buffer (yielding a 400 μg/mL solution of mRNA) in 65 mM pH 4 acetate buffer. The lipid components were dissolved in anhydrous ethanol at the relative ratios set forth in Table 28 below.

TABLE 28
Ratios of lipid blend solution.

| | | Ratio of Lipid to | | Theoretical LNP |
|---|---|---|---|---|
| | | mRNA (nmol | Concentration in | Lipid |
| | | lipid/100 μg | Lipid Solution | Composition |
| Lipid | Source | mRNA) | (mM) | (mol %) |
| Lipid 15 | — | 1,500 | 18 | 49.2449 |
| Cholesterol | Dishman | 1,200 | 14.4 | 39.3959 |
| | Netherlands | | | |
| DSPC | Avanti Polar | 300 | 3.6 | 9.849 |
| | Lipids, Alabama, | | | |
| | U.S. | | | |
| DPG-PEG(2000) | NOF America, | 46 | 0.55 | 1.5102 |
| | New York, U.S. | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directly mounted into the luer ports of the mixing cartridge. The two solutions were then mixed using the NanoAssemblr Ignite (the ratios, volumes, and flow rates can vary). The resulting suspension was held at room temperature for a minimum of 5 minutes before proceeding to ethanol removal and buffer exchange.

Following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with MBS exchange buffer. The LNP suspension (5 mL) was then loaded into the device and centrifuged at 500 RCF until the volume was reduced by half (2.5 mL). The suspension was then diluted with exchange buffer (2.5 mL of MBS) to bring the suspension back to the original volume. This process of two-fold concentration and two-fold dilution was repeated five additional times for a total of six discontinuous diafiltration steps. The retentate containing the LNPs in MBS was recovered from the centrifugal ultrafiltration device, mixed with sucrose to a final sucrose concentration of 10% w/v, and then filtered using a 0.2 μm PES syringe filter. The LNPs were either used right away, or stored frozen at −80° C. until further use.

### Example 42. Characterization of LNPS

This Example describes the characterization of LNPs produced in Example 41 above.

Samples of the LNPs produced in Example 41 above were characterized to determine the average hydrodynamic diameter, zeta potential, and mRNA content (total and dye-accessible mRNA). The hydrodynamic diameter was determined by dynamic light scattering (DLS) using a Zetasizer model ZEN3600 (Malvern Pananalytical, UK). The zeta potential was measured in 5 mM MES buffer at pH 5.5 and 5 mM HEPES buffer at pH 7.4 by laser Doppler electrophoresis using the Zetasizer.

RNA content of the nanoparticles was measured using Thermo Fisher Quant-iT RiboGreen RNA Assay Kit. Dye accessible RNA, which includes both un-encapsulated RNA and accessible RNA at the LNP surface, was measured by diluting the nanoparticles to approximately 1 μg/mL mRNA using HEPES buffered saline, and then adding Quant-iT reagent to the mixture. Total RNA content was measured by disrupting a nanoparticle suspension by dilution of the stock LNP batch (typically at ≥40 μg/mL RNA) in 0.5% Triton solution in HEPES buffered saline to obtain a 1 μg/mL RNA solution (final nominal concentration based on formulation input values) and subsequent heating at 60° C. for 30 minutes followed by addition of Quant-It reagent. RNA was quantified by measuring fluorescence at 485/535 nm, and concentration was determined relative to a contemporaneously run RNA standard curve. The results are set forth in Table 29.

TABLE 29
Results of LNP characterization.

| | | DLS Z- | | Zeta | Zeta | Dye- |
|---|---|---|---|---|---|---|
| | | Avg. | | Potential | Potential | Accessible |
| LNP Lot | mRNA | Diameter | DLS | at pH 5.5 | at pH 7.4 | mRNA |
| Number | Payload | (nm) | PDI | (mV) | (mV) | (%) |
| EXP23012510- | mCherry | 89 | 0.10 | 20.1 | 0.8 | 13 |
| NCS | | | | | | |

### Example 43. Preparation of Fab or VHH Conjugates to Enable T Cell Targeting

Fab or VHH targeting moieties were conjugated to DSPE-PEG (3.4k)-maleimide via covalent coupling between the maleimide group and a C-terminal cysteine in the heavy chain (HC). The protein (3-4 mg/mL), after buffer exchange into pH 7.4 phosphate buffered saline (PBS 137 mM NaCl, 2.7 mM KC1, 10 mM Na₂HPO₄, and 1.8 mM KH₂PO₄) with 5 mM ethylenediaminetetraacetic acid (EDTA), was reduced using 2 mM Tris(2-carboxyethyl)phosphine hydrochloride (TCEP) for 1 hour at room temperature. The reduced protein was isolated using a 7 kDa molecular weight cutoff (MWCO) SEC column to remove TCEP and buffer exchanged into fresh PBS with 5 mM EDTA.

The conjugation reaction was initiated by addition of a 10 mg/mL micellar suspension of DSPE-PEG-maleimide and 30 mg/mL DSPE-PEG-OCH3 (1:1 to 1:4 weight ratio is used depending on protein) in PBS. The conjugation reaction is carried out using 2-4 mg/mL protein and a 3.5 molar excess of maleimide at 37° C. for 2 hours followed by incubation at room temperature for an additional 12-16 hours.

The production of the resulting conjugate was monitored by HPLC, and the reaction was quenched in 2 mM cysteine. The resulting conjugate (DSPE-PEG-Fab) is isolated using a 100 kDa or 50 kDa MWCO Millipore Regenerated Cellulose membrane filtration using pH 7.0 HEPES buffer saline (25 mM HEPES, 150 mM NaCl) and stored at 4° C. prior to use. After quenching, the final micelle composition consists of a mixture of DSPE-PEG-Fab, DSPE-PEG-maleimide (cysteine terminated), and DSPE-PEG-OCH3. The ratio of the three components is approximately DSPE-PEG-Fab: DSPE-PEG-maleimide (cysteine terminated): DSPE-PEG-OCH3=1:2.45:3.45-10.35 (by mol)).

Generation of Nb-conjugates follows the same procedure but uses a 1:1:4 or 1:2:3 (VHH:DSPE-3.4K PEG-maleimide: DSPE-2KPEG-OCH3) molar ratio, as described in later examples.

### Example 44. Preparation of Fab or VHH Conjugates with Shortened DSPE-PEG LINKER

Fab or VHH targeting moieties were conjugated to DSPE-PEG (2k)-maleimide via covalent coupling between the maleimide group and a C-terminal cysteine in the heavy chain (HC) as described in Example 43 above.

### Example 45. Preparation of DSPE-PEG(3.4K)-15C01V8 Conjugate

15C01v8 (SEQ ID NO: 9, SEQ ID NO: 169, or SEQ ID NO: 44) was conjugated to DSPE-PEG (3.4k)-maleimide via covalent coupling between the maleimide group and a C-terminal cysteine in the heavy chain (HC) as described in Example 43 above.

### Example 46. Preparation of LNPS Containing T Cell Targeting Group

This Example describes the incorporation of an immune cell targeting conjugate into preformed LNPs.

LNPs from Example 41 above and DSPE-PEG-anti-CD8 VHH conjugate 15C01v8 from Example 45 (SEQ ID NO: 9, SEQ ID NO: 169, or SEQ ID NO: 44) (prepared using the methods described in Example 87 below) were combined in a 15 ml conical tube at a ratio of 0.084 g VHH conjugate per 1 g of mRNA and diluted to a final mRNA concentration of 0.2 mg/mL. The tube was placed into a ThermoMixer pre-heated to 37° C. and then mixed at 300 rpm for 4 hours at 37° C. The resulting targeted LNP suspension was subsequently filtered using a 0.2 μm PES syringe filter and then either used immediately or stored frozen at −80° C.

### Example 47. Preparation of LNPs by Microfluidic in-Line Mixing and TFF Using an Exemplary Ionizable Lipid

This example describes preparation of LNPs using scalable unit operations, namely in-line microfluidic mixing followed by tangential flow filtration (TFF) for ethanol removal and buffer exchange.

LNPs encapsulating an mRNA payload were prepared by mixing an aqueous mRNA solution and an ethanolic lipid blend solution using an in-line microfluidic mixing process. The mRNA (anti-CD19 CAR #1 (SEQ ID NO: 435 or SEQ ID NO: 49 with signal peptide), CAR #2 (SEQ ID NO: 436 or SEQ ID NO: 50 with signal peptide), or CAR #3 (SEQ ID NO: 437 or SEQ ID NO: 51 with signal peptide)) stock solution was diluted in pH 4.0 acetate buffer (yielding a 400 μg/mL solution of mRNA) in 65 mM pH 4.0 acetate buffer. The lipid components were dissolved in anhydrous ethanol at the relative ratios set forth in Table 30 below.

TABLE 30
Ratios of dissolved LNP components.

| | | Ratio of Lipid to | | Theoretical LNP |
|---|---|---|---|---|
| | | mRNA (nmol | Concentration in | Lipid |
| | | lipid/100 μg | Lipid Solution | Composition |
| Lipid | Source | mRNA) | (mM) | (mol %) |
| Lipid 15 | — | 1,500 | 18 | 49.2449 |
| Cholesterol | Dishman | 1,200 | 14.4 | 39.3959 |
| | Netherlands | | | |
| DSPC | Avanti Polar | 300 | 3.6 | 9.849 |
| | Lipids, Alabama, | | | |
| | U.S. | | | |
| DPG-PEG(2000) | NOF America, | 46 | 0.55 | 1.5102 |
| | New York, U.S. | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directly mounted into the luer ports of the mixing cartridge. The two solutions were then mixed at a 3:1 v/v ratio of mRNA solution (9 mL) to lipid solution (3 mL) at a total flow rate of 9 mL/min using the NanoAssemblr Ignite. This mixing process was performed three additional times, and the resulting LNP suspensions were pooled to yield 48 mL of LNP suspension at 0.3 mg/mL. The resulting suspension was held at room temperature for a minimum of 5 minutes before proceeding to ethanol removal and buffer exchange. Ethanol removal and buffer exchange were subsequently performed using constant volume tangential flow filtration (TFF).

Following mixing, ethanol removal and buffer exchange were performed on the resulting LNP suspension using a hollow fiber TFF module (mPES membrane with 300 kDa MWCO, Repligen, US) and MBS as the exchange buffer. Briefly, the TFF module was rinsed with DI water and pumped dry before use. The LNP mixture in ethanol/water solution was added to the feed reservoir, the TFF module was primed, and diafiltration with MBS was initiated by ramping up the peristaltic pump to the target flow rate and adjusting the retentate valve until the target transmembrane pressure (TMP) was reached. A shear rate of 8000 s-1 and a TMP of 3.5 psi were the target operating parameters for the system once diafiltration was initiated. Throughout the diafiltration process, the TMP was kept constant by adjusting the retentate valve. Permeate flux was monitored and was >20 LMH throughout diafiltration. Six diafiltration exchange volumes were performed, with samples set aside at the end of each diafiltration volume to later track the buffer exchange process. Final ethanol content was ≤0.8%, as measured by refractive index measurements on permeate samples, and pH measurements confirmed the buffer exchange into the desired diafiltration buffer. Upon the completion of six diafiltration volumes, a concentration of the resulting LNP suspension was subsequently performed.

The concentration of the LNP suspension to a target total mRNA concentration of ~0.8 mg/mL was performed using the same TFF module that was used during the buffer exchange process. TMP and flow rate during the buffer exchange process were maintained, and the suspension was allowed to concentrate by stopping the addition of diafiltration buffer to the retentate reservoir. The resulting LNP suspension was collected and filtered with a 0.2 um syringe filter. The suspension was then mixed with a sterile-filtered 49 wt % sucrose solution to a final sucrose concentration of 10% w/v and then stored frozen at −80° C.

Using the LNP characterization process in Example 42 above, the LNP batch was characterized to determine the average hydrodynamic diameter, zeta potential at pH 5.5 and 7.4, and mRNA content (total and dye-accessible). As seen in Table 31, the microfluidic mixing process with ethanol removal and buffer exchange by TFF results in ~100 nm particles exhibiting narrow polydispersity and good mRNA encapsulation (<10% dye accessible RNA).

TABLE 31
Results of LNP microfluidic mixing process.

| | DLS Z- | | Zeta | Zeta | | Dye- |
|---|---|---|---|---|---|---|
| | Avg. | | Potential | Potential | Total | Accessible |
| LNP Lot | Diameter | DLS | at pH 5.5 | at pH 7.4 | mRNA | mRNA |
| Number | (nm) | PDI | (mV) | (mV) | (mg/mL) | (mg/mL) |
| EXP22015234- | 97 | 0.11 | 22.0 | −0.4 | 0.61 | 0.05 |
| NX501 | | | | | | |

### Example 48. Physiochemical Properties (Pre- and Post-Insertion with AntiCD8 VHH Conjugate 15C01V8 of Lipid15 CAR RNA LNPs

LNPs from Example 46 above inserted with DSPE-PEG-anti-CD8 VHH conjugate 15C01v8 (SEQ ID NO: 9) were characterized as described in Example 42 above. Compared against the pre-insertion results summarized in Table 29 above, the insertion process led to a size increase of ~10 nm, a zeta potential decrease of ~3 mV, and a reduction in the dye-accessible mRNA content to ~5%.

### Example 49. Method for the Primary Human Cd8+ T Cell Transfection with DiI-C18-5DS Labelled LNPs

CD8+ T cells were isolated from frozen peripheral blood mononuclear cells using an EasySep Human T Cell Isolation Kit on a RoboSep automated cell isolation system from STEMCELL. CD8+ T cells were plated into a round bottom 96-well plate in RPMI cell culture media supplemented with glutamax, 10% fetal bovine serum, and 50 ng/ml IL-2. 100 μL of CD8+ T cell suspension was seeded per well at a density of 1M CD8+ T cells/mL (100K CD8+ T cells/well). CD8+ T cells were allowed to rest overnight in a 37° C. incubator, 5% CO₂, and then transfected by gently adding 10 μL of a 11 μg/mL (by mRNA) nanoparticle suspension, resulting in a final mRNA concentration of 1 μg/mL (unless otherwise noted). CD8+ T cells were gently mixed with a pipette and then incubated for 24 hours in a 37° C. incubator, 5% CO₂. After incubation, the CD8+ T cells were diluted with FACS buffer (BD 554657) and analyzed using a Novocyte Penteon Flow Cytometer (Agilent). Data were analyzed using FlowJo software from BD biosciences.

### Example 50. Method for the Primary Human T Cell Transfection with Cd19 Car LNPs

CD8+ T cells were isolated from frozen peripheral blood mononuclear cells (PBMCs) using an EasySep Human T Cell Isolation Kit on a RoboSep automated cell isolation system from STEMCELL. CD8+ T cells were plated into a round bottom 96-well plate in RPMI cell culture media supplemented with glutamax, 10% fetal bovine serum, and 50 ng/ml IL-2. 100 μL of CD8+ cell suspension was seeded per well at a density of 1 million CD8+ T cells/mL (100K CD8+ T cells/well). CD8+ T cells were allowed to rest for two hours in a 37° C. incubator, and then transfected by gently adding 10 μL of a 3.3 μg/mL (by mRNA) nanoparticle suspension, resulting in a final mRNA concentration of 0.33 μg/mL (unless otherwise noted). CD8+ T cells were gently mixed with a pipette and then incubated for 24 hours in a 37° C. incubator. After incubation, the CD8+ T cells were diluted with FACS buffer (BD 554657) and analyzed using a Novocyte Quanteon flow cytometer (Agilent). Data were analyzed using FlowJo software from BD biosciences.

### Example 51. Cd4 and Cd69 Staining Protocol Post Transfection

After 24 hours, cells were transferred to a 96-well conical bottom polypropylene plate and centrifuged at 350×g for 5 minutes. Supernatants were removed and transferred to a fresh conical bottom polypropylene plate for further cytokine analysis. Cells were washed by adding 200 μL FACS buffer (BD 554657), centrifuging at 350×g for 5 min, and then aspirating the supernatant from each well. BV421 anti-human CD69 (BioLegend 310930 clone FN50) and BV711 anti-human CD4 (BioLegend 344648 clone SK3) antibodies were diluted 100-fold by adding 100 μL of each antibody to 10 mL FACS buffer. 100 μL of the diluted antibody solution was added to each well and the plate was incubated at 4° C. for 30 minutes. The plates were then washed by centrifuging at 350×g for 5 min, removing the supernatant, re-suspending in 200 μL FACS buffer, centrifuging at 350×g for 5 min, and aspirating the supernatant from each well. Following the wash, cells were resuspended in 100 μL of FACS buffer and immediately analyzed by flow cytometry. Flow cytometry analysis was performed using a Novocyte Penteon (Agilent). Data were analyzed using FlowJo software from BD biosciences.

### Example 52. Method for Quantifying Secreted Cytokines by MSD

IFN-γ, TNF-alpha, Granzyme B, Granzyme A, and GM-CSF were assayed using a custom U-Plex MSD panel (Meso Scale Discovery). Briefly, 200 μL of each biotinylated antibody was added to 300 μL of the corresponding assigned linker and mixed by vortexing. After 30 minutes of incubation at room temperature, 200 μL of stop solution was added and the solution was mixed by vortexing. 600 μL of each U-PLEX Linker-coupled antibody solution was combined into a single tube and mixed by vortexing. The total volume was brought up to 6 mL with Stop solution to reach a final 1× concentration. 50 μL of the prepared multiplex coating solution was added to each well in the U-PLEX 96-well plate and the plate was sealed and incubated at room temperature with shaking (700 rpm) for one hour. Following coating, the plate was washed 3 times using 150 μL/well of 1×MSD Wash Buffer. Calibrator standards were prepared according to the manufacturer's instructions using a 4-fold dilution schema. 50 μL of sample supernatants (diluted 3-fold) and calibrator standards were added to the respective wells. The plate was sealed and incubated at room temperature with shaking (700 rpm) for 2 hours. The plate was then washed 3 times using 150 μL/well of 1×MSD Wash Buffer. Detection antibody stock was diluted 100× to reach a 1× concentration and 50 μL was added to each well following 1 hour incubation at room temperature with shaking (700 rpm). The plate was then washed 3 times using 150 μL/well of 1×MSD Wash Buffer and 150 μL of MSD GOLD Read Buffer was added to each well before immediately analyzing using a MESO QuickPlex SQ 120 MM instrument. The cytokine concentrations were quantified relative to the respective standard curves. The data was analyzed using the MSD Discovery Workbench software and plotted in Graph Pad Prism.

### Example 53. CD19 CAR Staining Protocol Using Directly Labeled CD19 Antigen

Cells were transferred to a 96-well conical bottom plate and washed by centrifuging at 350×g for 5 minutes followed by resuspension in FACS buffer (BD 554657). Anti-CD19 CAR protein was detected by staining with recombinant CD19 antigen conjugated to AF647 (Acro Biosystems CD19-HA2H6) at room temperature for 20 minutes. Following staining, cells were washed twice by centrifugation at 350×g for 5 minutes followed by resuspension in FACS buffer. Cells were analyzed by FACS using a Novocyte Quanteon flow cytometer (Agilent).

### Example 54. CD19 CAR Staining Protocol Using Indirect Detection with Biotinylated CD19 Antigen and Anti-Biotin Antibody

Cells were transferred to a 96-well conical bottom plate and washed by centrifuging at 350×g for 5 minutes followed by resuspension in FACS buffer (BD 554657). CD19 CAR protein was detected by staining with a biotinylated anti-CD19 CAR detection reagent (Miltenyi 130-129-550) at room temperature for 20 minutes. Following primary staining, cells were washed twice by centrifugation at 350×g for 5 minutes followed by staining with a secondary anti-biotin antibody conjugated to APC (Miltenyi 130-110-952) or PE (130-110-951) at room temperature for 20 minutes. Following secondary staining, cells were washed twice by centrifugation at 350×g for 5 minutes followed by resuspension in FACS buffer. Cells were analyzed by FACS using a Novocyte Quanteon flow cytometry (Agilent).

### Example 55. In Vitro LNP Car Transfection into Primary Human T Cells and Nalm6, Raji, or K562 Co-Culture for Assessment of CAR T Cell Function

CD8+ T cells were isolated from human PBMCs by using EasySep Human CD8+ T cell isolation kit (catalog #17953) according to manufacturer's instructions. Isolated CD8+ T cells were rested overnight in T cell media (RPMI+Glutamax, Gibco, catalog #61870-036) with 10% heat inactivated fetal bovine serum (HI-FBS, Gibco, catalog #16140-071) supplemented with 100 IU human IL-2 (Miltenyi catalog #130-097-748) at a density of 500,000 cells/well in a 96 well plate. CD8+ T cells were then transfected with LNPs at an mRNA concentration of 0.33 μg/mL. CAR expression was detected 18 hrs post-LNP transfection by flow cytometry. Before co-culture with Nalm6, Raji, or K562 cells, the transfected CD8+ T cells were washed twice with T cell media. Nalm6, Raji, and K562 cells were stained with cell trace violet (CTV, Thermo Fisher, catalog #C34571) and co-cultured with different effector to target (E:T) ratios of CD8+ T cells in a 96 well flat-bottom plate format and incubated at 37° C. for 72 hours. 72 hours post co-culture, the cells were stained with live/dead stain (eBioscience Fixable viability dye eFluor 780, Invitrogen catalog #2290917) and analyzed by flow cytometry. Data was analyzed with FlowJo and Graph Pad prism.

### Example 56. Method for Primary NHP T Cell Transfection with CAR LNPS

Rhesus CD8+ T cells were isolated from frozen rhesus peripheral blood mononuclear cells using an EasySep NHP T Cell Isolation Kit on a RoboSep automated cell isolation system from STEMCELL. T cells were plated into a round bottom 96-well plate in RPMI cell culture media supplemented with glutamax, 10% fetal bovine serum, and 50 ng/ml human IL-2. 100 μL of cell suspension was seeded per well at a density of 1M T cells/mL (100 K T cells/well). Cells were allowed to rest overnight in a 37° C. incubator, 5% CO₂, and then transfected by gently adding 10 μL of a 11 μg/mL (by mRNA) nanoparticle suspension, resulting in a final mRNA concentration of 1 μg/mL (unless otherwise noted). Cells were gently mixed with a pipette and then incubated for 24 hours in a 37° C. incubator, 5% CO₂. After incubation the cells were diluted with FACS buffer (BD 554657) and analyzed using a Novocyte Penteon Flow Cytometer (Agilent). Data were analyzed using FlowJo software from BD biosciences.

### Example 57. Method for Primary Human and NHP PBMC Transfection with CAR LNPs

Frozen Rhesus, Cyno, and Human peripheral blood mononuclear cells (PBMCs) were thawed and plated into a round bottom 12-well plate in RPMI cell culture media supplemented with glutamax and 10% fetal bovine serum. 500 μL of cell suspension was seeded per well at a density of 1 million PBMCs/mL (500 K PBMCs/well). Cells were transfected by gently adding 10 μL of a 16.5 μg/mL (by mRNA) nanoparticle suspension, resulting in a final mRNA concentration of 0.33 μg/mL (unless otherwise noted). Cells were gently mixed with a pipette and then incubated for 24 hours in a 37° C. incubator at 5% CO₂. After incubation, the cells were diluted with FACS buffer (BD 554657) and analyzed using a Novocyte Penteon Flow Cytometer (Agilent). Data were analyzed using FlowJo software from BD biosciences.

### Example 58. Human and NHP PBMC Staining Protocol Post-Transfection

After 24 hours, cells from Example 54 above were transferred to a 96-well conical bottom polypropylene plate and centrifuged at 350×g for 5 minutes. Supernatants were removed and transferred to a fresh conical bottom polypropylene plate for further cytokine analysis. Cells were washed by adding 200 μL FACS buffer (BD 554657), centrifuging at 350×g for 5 min, and then aspirating the supernatant from each well. Cells were first Fc-blocked for 10 minutes at 4° C. Then BUV395 anti-human CD20 (BD 563782 clone 2H7), BUV496 anti-human CD69 (BD 750214 clone FN50), BUV805 anti-human CD45RA (BD 742052 clone 5H9), BV421 anti-human CD8α (Biolegend 344748 clone SK1), BV650 anti-human CD14 (BD 563419 clone M5E2), BV711 anti-human CD95 (BD 563132 clone DX2), BV786 anti-human CD4 (BD 563914 clone L200), AF488 anti-human CCR7 (Biolegend 353206 clone G043H7), PE anti-G4S (CST 38907 clone E702V), APC anti-human CD159a (Beckman Coulter A60797 clone Z199), and AF700 anti-human CD3 (BD 557917 clone SP34-2) antibodies were diluted 100-fold by adding 100 μL of each antibody to 10 mL FACS buffer. 100 μL of the diluted antibody solution was added to each well and the plate was incubated at 4° C. for 30 minutes. The plates were then washed by centrifuging at 350×g for 5 min, removing the supernatant, re-suspending in 200 μL FACS buffer, centrifuging at 350×g for 5 min, and aspirating the supernatant from each well. Following the wash, cells were resuspended in 150 μL of FACS buffer and immediately analyzed by flow cytometry. Flow cytometry analysis was performed using a Cytek Aurora (Cytek Biosciences). Data were analyzed using FlowJo software from BD biosciences. Dead cells were determined based on a Live/Dead stain using eFluor780 fixable viability dye (Invitrogen 65-0865-18).

### Example 59. Method for Transfecting HEK293T Cells with Car Plasmid Via Lipofectamine

HEK293T cells were seeded in DMEM media into a 48-well plate to reach 70-90% confluency at the time of transfection. 30 minutes prior to transfection the media was replaced with Opti-MEM. For each reaction, 0.6 μL Lipofectamine 3000 Reagent was diluted in 20 μL Opti-MEM Medium and vortexed. 0.4 μg of CAR plasmid DNA was diluted in 20 μL Opti-MEM and 0.8 μL of P3000 reagent was added. 20 μL of the diluted CAR plasmid DNA solution was added to 10 μL of the diluted Lipofectamine 3000 reagent. The solution was then incubated for 15 minutes at room temperature before adding 10 μL of the DNA-lipid complex solution to the respective well containing HEK293T cells. Transfected HEK293T cells were incubated for 24 hours at 37° C. until analysis by flow cytometry.

### Example 60. Jurkat T Cell Transfection with Car Plasmid Via Electroporation

Jurkat T cells were harvested from culture and counted. For each reaction, 100K Jurkat T cells were resuspend in 20 μL SE 4D-Nucleofector™ X Solution (Lonza) and 0.6 μg CAR plasmid DNA was added to the cells and co-incubated for 3 minutes at room temperature. The solution was then transferred to a 16-well electroporation strip (Lonza) and electroporated using the CL-120 pulse code on a 4D-Nucleofector system (Lonza). Following electroporation, Jurkat T cells were immediately recovered in 180 μL pre-warmed recovery media (RPMI+20% FBS), seeded into a 96-well plate at 20K cells per well, and incubated over night at 37° C. Following recovery, transfected Jurkat T cells were co-cultured with Raji cells at varying effector to target cell ratios (E:T) and then incubated at 37° C. for 24 hours before staining and analyzing by flow as described in Example 51 above.

### Example 61. Transfection of Jurkat T Cells and Primary T Cells with mRNA Via Electroporation

Jurkat T cells were harvested from culture and counted, and primary T cells were isolated from human PBMC. For each reaction, 100 K Jurkat T cells or primary T cells were resuspend in 20 μL SE 4D-Nucleofector™ X Solution (Lonza) and 0.6 μg CAR mRNA was added to the cells and co-incubated for 3 minutes at room temperature. The solution was then transferred to a 16-well electroporation strip (Lonza) and electroporated using the CL-120 pulse code on a 4D-Nucleofector system (Lonza). Following electroporation, cells were immediately recovered in 180 μL pre-warmed recovery media (RPMI with 20% FBS), seeded into a 96-well plate at 20K cells per well, and incubated over night at 37° C. Following recovery, transfected Jurkat T cells or primary T cells were co-cultured with Raji cells at varying effector to target cell ratios (E:T) and then incubated at 37° C. for 24 hours before staining and analyzing by flow as described in Example 51 above.

### Example 62. Cd19 Car-Mediated Cd69 Activation in Jurkat T Cells and Primary T Cells Transfected with Car mRNA Encoding Various Anti-Cd19 Car Clones and Car Cassettes

This example compares the CD69 signal in Jurkat T cells and in primary T cells resulting from anti-CD19 CAR mRNA transfection and co-culture with CD19-expressing Raji cells. mRNA encoding various anti-CD19 CARs were transfected into Jurkat T cells or into primary T cells and then co-cultured with Raji cells as described in Example 55 above. The level of CD69 expression was assessed as described in Example 51 above. Increased CD69 expression with a signal/noise >10.5 was observed in Jurkat T cells transfected with mRNA encoding clone αCD19 #2 incorporated into a CAR cassette containing a CD8alpha-derived hinge domain of 46 amino acids in length, clone αCD19 #6 incorporated into CAR cassettes containing a CD28-derived hinge domain of 40 amino acids in length, a CD8alpha-derived hinge domain of 46 amino acids in length, and a CD8alpha-derived hinge domain of 66 amino acids in length, clone αCD19 #9 incorporated into a CAR cassette containing a CD8alpha-derived hinge domain of 66 amino acids in length, and clone αCD19 #15 incorporated into a CAR cassette containing a CD8alpha-derived hinge domain of 66 amino acids in length (see FIGS. 31A-31B and Table 32 below).

TABLE 32
Exemplary anti-CD19 CAR signal-to-noise ratio in Jurkat T cells.

| | CD28- | CD8-26 | CD8-46 | CD8-66 |
|---|---|---|---|---|
| Clone | 40AAs | AAs | AAs | AAs |
| αCD19#1 | 9.23 | NA | 7.93 | 7.58 |
| αCD19#2 | 9.12 | NA | 10.57 | 8.15 |
| αCD19#3 | 9.48 | NA | 7.97 | 7.19 |
| αCD19#4 | 10.10 | 4.27 | 7.12 | 6.13 |
| αCD19#5 | 8.21 | 6.85 | 8.67 | 8.47 |
| αCD19#6 | 13.11 | 5.77 | 11.24 | 11.12 |
| αCD19#7 | 8.87 | 6.31 | 7.31 | NA |
| αCD19#8 | 6.69 | 7.35 | 9.76 | 7.55 |
| αCD19#9 | 10.10 | 5.15 | 9.81 | 10.71 |
| αCD19#10 | 9.13 | 6.70 | 8.61 | 8.09 |
| αCD19#11 | 7.40 | 6.35 | 6.62 | 7.15 |
| αCD19#12 | NA | 2.84 | 7.48 | 9.12 |
| αCD19#13 | NA | NA | 6.79 | 7.21 |
| αCD19#14 | NA | 1.91 | 7.658 | 8.05 |
| αCD19#15 | NA | 3.83 | 7.98 | 11.18 |
| αCD19#16 | NA | NA | 9.61 | 4.45 |

### Example 63. Transfection of Primary Human T Cells with mRNA Via Electroporation and Subsequent Co-Culturing with Target Cells

Primary human CD8+ T cells were isolated from frozen peripheral blood mononuclear cells (PBMCs) using an EasySep Human T Cell Isolation Kit on a RoboSep automated cell isolation system from STEMCELL. CD8+ T cells were plated into a 6-well plate in RPMI cell culture media supplemented with glutamax, 10% fetal bovine serum, and 50 ng/ml IL-2. 3 mL of CD8+ T cell suspension was seeded per well at a density of 1 million CD8+ T cells/mL (3 million CD8+ T cells/well). CD8+ T cells were allowed to rest overnight in a 37° C. incubator at 5% CO₂. For each reaction, 1 million CD8+ T cells were harvested and resuspended in P3 electroporation buffer (Lonza), and 1 μg of mRNA was then added to the CD8+ T cell suspension before transferring the cell-mRNA solution to an electroporation cuvette (Lonza). CD8+ T cells were electroporated using the FI-115 pulse code on a 4D-Nucleofector system (Lonza). Following electroporation, CD8+ T cells were immediately recovered in 500 μL pre-warmed recovery media (RPMI with 20% FBS and 50 ng/ml IL-2), seeded into a 12-well plate, and incubated at 37° C. for 1 hour. For CAR expression studies, CD8+ T cells were allowed to rest for 24 hours until the time of analysis where cells were then stained as described in Example 57 and analyzed by flow cytometry using a Novocyte Penteon Flow Cytometer (Agilent). For cytotoxicity assays, electroporated CD8+ T cells were co-cultured with target Raji or Nalm6 cells at varying effector to target cell ratios (E:T) and then incubated at 37° C. for 48 hours before staining and analyzing by flow as described in Example 58 above.

### Example 64. Cd19-Car Expression in Primary Human T Cells Transfected by Electroporation with mRNA Encoding Anti-Cd19 Car Constructs

This example compares the anti-CD19 CAR protein expression levels after 24 hours in primary human CD8+ T cells resulting from CAR mRNA transfection. mRNA encoding various anti-CD19 CAR constructs were transfected into human CD8+ T cells as described in Example 63 above. The level of anti-CD19 CAR expression was assessed using the method described in Example 55 above. mRNA encoding the clinically validated FMC63 clone was used as a benchmark control. CD8+ T cells transfected with mRNA encoding clone αCD19 #6 incorporated into a CAR cassette containing a CD28-derived hinge domain of 40 amino acids in length showed the highest CAR expression levels of the constructs tested (see FIG. 32).

### Example 65. Protocol for In Vivo Cd8+ T Cell Reprogramming in Human PBMC-Engrafted NSG-Mice

The following is a standard procedure for in vivo reprogramming of immune cells with anti CD8-targeted LNPs encapsulating CAR-encoding mRNA.

### Mice Strains and Humanization

NSG-MHCI/II double-Knockout (DKO) mice were obtained 6-8 weeks old female mice were engrafted with 10 million human PBMC from a donor with a pre-characterized donor profile for Immuno-Oncology studies via tail vein injection. Body weight was monitored three times a week and blood samples were collected at Day 10-12 post PBMC inoculation to evaluate PBMC engraftment.

Evaluation of Human T cell Engraftment in Humanized-Mice

75-100 μl of blood was collected by submandibular bleed from each mouse. Red blood cells (RBCs) were lysed using ACK lysis buffer, RBC lysis solution following protocol as instructed by the manufacturer (Gibco A1049201 A09777). Cells were stained with mCD45, hCD45, hCD3, hCD19, hCD20, hCD22, hCD8 and hCD4 to quantify the engraftment of human cells. A volumetric acquisition was set on Cytek Aurora to enable the determination of absolute cell counts. At day 9-12 days post PBMC injection, engraftment of huCD45+ is 5-45% of total gated alive cells.

### In Vivo Reprogramming of CD8+T Cells to Anti-CD19 CAR T Cells

At day 14-15 post PBMC engraftment mice were treated with one single dose of LNP loaded with mRNA (CAR T-construct or mCherry) via tail vein injection. For single-dose proof of concept studies, mice were bled at 6 hr post LNP injection and sacrificed at 24 hrs post dosing. Whole blood (WB) and tissues (Spleen and Bone Marrow) were collected to evaluate CAR expression and in vivo function. CAR T cell in vivo function was evaluated by the % of B cell depletion detected in circulation, spleen and BM. B cell depletion is a sign of functional CAR T cell persistence. For lipid analysis, plasma, kidney, and liver were weighed and frozen for downstream analysis at the takedown.

### Tissue and Blood Sample Collection

In life blood collection (PBMC engraftment check or CAR expression check in WB post 6 hr LNP injection) was performed via submandibular bleed. For submandibular blood collection, the mandibular vein was punctured by an appropriate sterile animal lanzet and blood was collected into an EDTA-tube (Sarstedt microvette 50020.1341.102). Plasma was collected by centrifuging at 2000 g for 10 min and then stored at −80° C.

Terminal blood collection was performed by cardiac puncture post CO₂ euthanasia 24 hrs post LNP injection. Spleen and Bone Marrow were harvested and processed for single-cell isolation and flow cytometry analysis. Single cell preparations from spleen were generated in a gentleMACS™ C Tubes (Miltenyi Biotec 130-093-237) and homogenized by gentleMACS Program m_spleen_01 according to the manufacturer's instructions. Tissues were resuspended and washed through a pre-rinsed 70 μm filter. Pelleted cells were resuspended in 3 mL of ACK Lysing Buffer and incubate for 10 minutes at 4C to remove RBC based per the vendor's protocol. The washed cells were resuspended in 1 ml BD Pharmingen™ Stain Buffer (BSA) (554657). 100 ul of cell suspensions were transferred to a 96 well U bottom plate for FACS acquisition.

Bone marrow was isolated by flushing both sides of the femurs with cold PBS. Liver and the left side of the kidney were weighed and frozen for lipid analysis.

### Immunophenotyping Analysis

Immune cells from blood and all the above tissues were processed with ACK buffer (Gibco, A1049201), RBC lysis buffer as per manufacturing instructions. Immune cells were stained with live/dead fixable dye and surface markers with standard flow analysis protocol as shown in the below panel in Table 33. The Cytek Aurora spectral flow cytometer was used to assess the frequencies of live cells and determine CAR expression and B cell depletion.

TABLE 33
Staining antibodies for immunophenotyping analysis.

| | | | | | | Target |
|---|---|---|---|---|---|---|
| Marker | Clone | Fluorophore | Vendor | Catalog # | Size | Species |
| Viability | NA | LD Blue | ThermoFisher | L34962 | 1000 test | N/A |
| CD45RA | HI100 | BUV395 | BD | 740298 | 100 test | human |
| CD3 | UCHT1 | BUV496 | BD | 612940 | 100 test | human |
| CD56 | NCAM16.2 | BUV563 | BD | 612928 | 100 test | human |
| CD19 | HIB19 | BUV661 | BD | 741604 | 100 test | human |
| CD4 | SK3 | eFluor 450 | eBioscience | 48-0047-41 | 100 test | human |
| CD14 | M5E2 | BV510 | BioLegend | 301842 | 100 test | human |
| HLA-DR | L243 | BV570 | BioLegend | 307638 | 100 test | human |
| CD16 | 3G8 | BV650 | BioLegend | 302042 | 100 test | human |
| CCR7 | G043H7 | BV711 | BioLegend | 353228 | 100 test | human |
| CD20 | 2H7 | BV786 | BD | 568713 | 100 test | human |
| CD8 | SK1 | FITC | BioLegend | | 100 test | human |
| hCD45 | F38-2E2 | AF532 | BioLegend | 345022 | 100 test | human |
| CD27 | O323 | PerCP-eF710 | Invitrogen | 46-0279-42 | 100 test | human |
| CD69 | FN50 | PE-Cy5 | BioLegend | 310908 | 100 test | human |
| CD22 | BC96 | PE-Cy7 | BioLegend | 302612 | 100 test | human |
| CD19-Ag- | N/A | N/A | Miltenyi | 130-129-550 | 20 test | human |
| biotin | | | | | | |
| Anti- | N/A | APC | Miltenyi | 130-110-952 | 100 test | N/A |
| biotin | | | | | | |
| CD62L | DREG-56 | AF700 | BioLegend | 304820 | 100 test | human |
| mCD45 | 30-F11 | APC-Cy7 | BD | 557659 | 100 test | mouse |
| CD38 | HIT2 | APC-Fire810 | BioLegend | 303550 | 100 test | human |

### Example 66. In Vitro Protein Expression (GFP) in Primary Human T Cells of Lipids 10, 15, 16, 24, 26 and Comparator Lipid ACD8 Targeted LNPS (ALC-0315) Stored at 4° C. And after One Freeze-Thaw Cycle (Post −80° C. Storage)

This example compares the GFP protein expression resulting from LNP's derived from Lipids 10, 15, 16, 24, and 26 to LNPs made using comparator lipids ALC-0315. Nanoparticles bearing GFP encoding mRNA (and optionally a fluorescent dye label (DiI-C18-5DS)) were produced using the microfluidic mixing and buffer exchange processes described in Example 41 above. An anti-CD8 VHH-conjugate was incorporated into the parent LNPs to obtain the final Nanobody®-targeted LNP formulation using the process described in Example 43 above. Particles thus produced were tested in vitro in primary human CD8+ T cells to assess reporter RNA expression as described in Example 49 above.

At 1 μg/mL and 0.25 μg/mL, a similar number of T cells showed LNP association as determined by the % DiI+ T cells (FIG. 33A). At the 1 μg/mL dose, DiI MFI values for ALC-0315 showed a drop from 1445.8 to 870.1 (FIG. 33B and Table 34) after one freeze/thaw cycle. This was not observed for Lipid 15 where the change in Dil MFI was less remarkable, 1096.3 to 1040.8 after 1 freeze/thaw cycle, indicating that LNPs based on Lipid 15 has improved freeze/thaw stability over ALC-0315 LNPs.

TABLE 34
DiI MFI values of Lipids 10, 15, 16, 24, and 26 and comparator lipid ALC-0315
at mRNA doses 1 ug/mL and 0.25 ug/mL and fold over background.

| | | | | | | | Fold over | Fold over |
|---|---|---|---|---|---|---|---|---|
| | | | | | | | background | background |
| DiI MFI | 1 ug/mL | | 0.25 ug/mL | | 0 ug/mL | | (1 ug/mL) | (0.25 ug/mL) |
| Lipid 10, 4 C. | 10594 | 11500 | 5806 | 4945 | | | 873.3 | 424.9 |
| Lipid 10, FT | 5940 | 6012 | 2250 | 2185 | | | 472.4 | 175.3 |
| Lipid 15, 4 C. | 14074 | 13663 | 10963 | 11222 | | | 1096.3 | 876.9 |
| Lipid 15, FT | 13447 | 12885 | 8775 | 6821 | | | 1040.8 | 616.4 |
| Lipid 16, 4 C. | 12258 | 12341 | 9529 | 4358 | | | 972.3 | 548.9 |
| Lipid 16, FT | 12086 | 12111 | 8716 | 4525 | | | 956.4 | 523.4 |
| Lipid 24, 4 C. | 10710 | 10675 | 6110 | 3449 | | | 845.3 | 377.8 |
| Lipid 24, FT | 9442 | 9357 | 5428 | 2838 | | | 743.0 | 326.7 |
| Lipid 26, 4 C. | 13513 | 13953 | 4873 | 3876 | | | 1085.6 | 345.8 |
| Lipid 26, FT | 12513 | 11600 | 5000 | 2970 | | | 953.1 | 315.0 |
| ALC-0315, 4 C. | 18332 | 18247 | 10661 | 4898 | | | 1445.8 | 615.0 |
| ALC-0315, FT | 11467 | 10546 | 7502 | 4188 | | | 870.1 | 462.1 |
| PBS | | | | | 12.1 | 13.2 | | |

At the 1 μg/mL dose, a similar number of CD8+ T cells were transfected with Lipid 15 and ALC-0315 LNPs, however, at the 0.25 μg/mL dose, a larger fraction of T cells were GFP+ with Lipid 15 LNPs (FIG. 33C) and the GFP MFI values were consistently increased at all doses suggesting more efficient cytosolic access/cytosolic release of encapsulated GFP mRNA relative to ALC-0315 LNPs (FIG. 33D). The performance levels and relative order of LNP efficiency were well preserved for Lipid 15 LNPs tested after 1 freeze/thaw cycle (Post-80° C. storage) as illustrated by the comparison of % GFP+ cells and GFP MFI values before and after −80° C. storage (FIG. 33C, FIG. 33D, and Table 35).

TABLE 35
GFP MFI values of Lipids 10, 15, 16, 24, and 26 and comparator lipid ALC-0315
at mRNA doses 1 ug/mL and 0.25 ug/mL and fold over background.

| | | | | | | | Fold over | Fold over |
|---|---|---|---|---|---|---|---|---|
| | | | | | | | background | background |
| GFP MFI | 1 ug/mL | | 0.25 ug/mL | | 0 ug/mL | | (1 ug/mL) | (0.25 ug/mL) |
| Lipid 10, 4 C. | 555 | 558 | 337 | 300 | | | 44.0 | 25.2 |
| Lipid 10, FT | 415 | 393 | 216 | 218 | | | 31.9 | 17.2 |
| Lipid 15, 4 C. | 1153 | 1003 | 811 | 799 | | | 85.2 | 63.6 |
| Lipid 15, FT | 1035 | 1014 | 777 | 718 | | | 81.0 | 59.1 |
| Lipid 16, 4 C. | 785 | 753 | 629 | 514 | | | 60.8 | 45.2 |
| Lipid 16, FT | 833 | 717 | 641 | 516 | | | 61.3 | 45.7 |
| Lipid 24, 4 C. | 193 | 187 | 150 | 141 | | | 15.0 | 11.5 |
| Lipid 24, FT | 204 | 192 | 152 | 142 | | | 15.7 | 11.6 |
| Lipid 26, 4 C. | 754 | 727 | 560 | 484 | | | 58.5 | 41.3 |
| Lipid 26, FT | 779 | 734 | 568 | 460 | | | 59.8 | 40.6 |
| ALC-0315, 4 C. | 901 | 842 | 684 | 503 | | | 68.9 | 46.9 |
| ALC-0315, FT | 670 | 625 | 524 | 414 | | | 51.2 | 37.1 |
| PBS | | | | | 133 | 136 | | |

### Example 67. Viability, LNP Association (Measured as DiI-Dye Fluorescence), and GFP Expression in Human Cd8+ T Cells Transfected with ACD8 (1705F, 2205F, 15C01, 1333F, 988F, TRX2) or Non-Binding (Mutated OKT8) Targeted DLN-KC2-DMA LNPs

This example compares the viability, Dil association, and GFP protein expression resulting from DLn-KC2-DMA LNPs with CD8-targeting moieties 1705F, 2205F, 15C01 (SEQ ID NO: 1), 1333F, 988F, and TRX2. Nanoparticles bearing GFP encoding mRNA (and a fluorescent dye label (DiI-C18-5DS)) were produced using the microfluidic mixing and buffer exchange processes described in Example 41 above. An αCD8 Fab or VHH conjugate was incorporated into the parent LNPs to obtain the final antibody targeted LNP formulation using the process described in Example 43 above. Particles thus produced were tested in vitro in primary human CD8+ T cells to assess reported gene expression as described in Example 49 above.

All targeting moieties tested showed a similar level of T cell viability to the untreated control 24 hours post-transfection (FIG. 34-1A). At all dose levels, 15C01 (SEQ ID NO: 1) LNPs outperformed other tested targeting moieties both in terms of the fraction of DiI+ T cells (reflected by % DiI+ cells) (FIG. 34-1B), the amount of LNP association per cell (reflected by Dil MFI values) (FIG. 34-1C), fraction of GFP+ T cells (reflected by % GFP+ cells) (FIG. 34-1D), and the copies of GFP protein produced on a per cell basis (reflected by GFP MFI values) (FIG. 34-1E). 15C01 (SEQ ID NO: 1) LNPs resulted in roughly 18-fold increased GFP MFI values over the untreated control with TRX2 LNPs resulting in an 8-fold increase in GFP MFI over the untreated control, suggesting more efficient receptor-mediated endocytosis with 15C01 (SEQ ID NO: 1) LNPs relative to TRX2 LNPs.

### Example 68. In Vitro LNP Association (Measured as DiI-Dye Fluorescence) and Protein Expression (GFP) in Primary Human Cd8+ T Cells Transfected with Lipid 15 LNPS Inserted with ACD8 (15C01V8 or TRX2), ACD3 (SP34), or Non-Binding (Mutated OKT8) Targeting Moieties

This example compares Dil association and GFP protein expression resulting from Lipid 15 LNPs with CD8-targeting moieties (15C01v8 or TRX2) and CD3-targeting moieties (SP34) at various dose levels. Nanoparticles bearing GFP encoding mRNA (and a fluorescent dye label (DiI-C18-5DS)) were produced using the microfluidic mixing and buffer exchange processes described in Example 41 above. An αCD8 Fab or VHH conjugate or αCD3 Fab was incorporated into the parent LNPs to obtain the final antibody targeted LNP formulation using the process described in Example 43 above. Particles thus produced were tested in vitro in primary human CD8+ T cells to assess LNP association and reporter protein expression as described in Example 49 above.

At dose levels <1 μg/mL, 15C01v8 LNPs outperformed other tested targeting moieties in terms of the fraction of DiI+ T cells (reflected by % DiI+ cells) (FIG. 35A). 15C01v8 was superior to other tested targeting moieties at all dose levels in terms of DiI MFI (FIG. 35B and Table 37).

TABLE 37
DiI MFI fold over untreated background values of anti-CD3-
and anti-CD8- targeted Lipid 15 LNPs
at mRNA doses 3, 1, 0.33, 0.11, and 0.037 ug/mL.

| DiI MFI | | | | | |
|---|---|---|---|---|---|
| (Fold over | | | | | 0.037 |
| background) | 3 ug/mL | 1 ug/mL | 0.33 ug/mL | 0.11 ug/mL | ug/mL |
| 15C01v8 | 10714.6 | 9819.6 | 3026.3 | 1151.4 | 114.6 |
| mutOKT8 | 5.6 | 3.0 | 2.0 | 1.1 | 1.0 |
| SP34 | 3037.3 | 1146.8 | 280.5 | 27.5 | 7.6 |
| TRX2 | 909.5 | 493.5 | 111.5 | 55.7 | 37.5 |

Similarly, at dose levels <1 μg/mL, 15C01v8 LNPs outperformed other tested targeting moieties in terms of the fraction of GFP+ T cells (reflected by % GFP+ cells) (FIG. 35C). 15C01v8 was superior to other tested targeting moieties at all dose levels in terms of GFP MFI (FIG. 35D and Table 38).

TABLE 38
GFP MFI fold over untreated background values of anti-CD3-
and anti-CD8- targeted Lipid 15 LNPs at
mRNA doses 3, 1, 0.33, 0.11, and 0.037 ug/mL.

| GFP MFI | | | | | |
|---|---|---|---|---|---|
| (Fold over | | | | | 0.037 |
| background) | 3 ug/mL | 1 ug/mL | 0.33 ug/mL | 0.11 ug/mL | ug/mL |
| 15C01v8 | 58.8 | 53.7 | 23.5 | 11.5 | 2.4 |
| mutOKT8 | 1.0 | 0.7 | 0.7 | 0.7 | 0.7 |
| SP34 | 53.0 | 38.5 | 9.3 | 1.6 | 1.1 |
| TRX2 | 4.1 | 2.3 | 1.4 | 1.3 | 1.2 |

### Example 69. In Vitro LNP Association (Measured as DiI-Dye Fluorescence) and Protein Expression (GFP) in Primary Human Cd8+ T Cells Transfected with Lipid 15 LNPS Inserted with ACD8 (15C01V8 or TRX2), ACD3 (SP34), or Non-Binding (Mutated OKT8) Targeting Moieties at Various Time Points

This example compares DiI association and GFP protein expression resulting from Lipid 15 LNPs with CD8-targeting moieties (15C01v8 or TRX2) and CD3-targeting moieties (SP34) at various time points. Nanoparticles bearing GFP encoding mRNA (and a fluorescent dye label (DiI-C18-5DS)) were produced using the microfluidic mixing and buffer exchange processes described in Example 41 above. An αCD8 Fab or VHH conjugate or αCD3 Fab was incorporated into the parent LNPs to obtain the final antibody targeted LNP formulation using the process described in Example 43 above. Particles thus produced were tested in vitro in primary human CD8+ T cells to assess LNP association and reporter protein expression as described in Example 49 above, but with the Dil signal and GPF expression being assessed at the various time points by utilizing the live cell imaging SX5 Incucyte system.

Cells treated with 15C01v8- and SP34-targeted LNPs reached a maximum in GFP expression around the same time point (~50 hours) as depicted by Green Integrated Intensity. However, cells treated with 15C01v8-targeted LNPs showed increased durability of GFP expression compared to cells treated with SP34-targeted LNPs during the time course tested (up to 240 hours) indicating that 15C01v8-targeted LNPs can deliver the mRNA payload to CD8+ T cells over extended time periods (FIG. 36A). Cells treated with 15C01v8-targeted LNPs showed increased Dil signal compared to SP34- and TRX2-targeted LNPs at all time points tested as depicted by NIR Integrated Intensity (FIG. 36B) indicating increased LNP association of 15C01v8-targeted LNPs compared to other targeting moieties tested.

### Example 70. LNP Association (Measured as DiI-Dye Fluorescence) and GFP Expression in Human Cd8+ T Cells Transfected with KC3 LNPs Containing Different Densities of ACD8 (15C01 and TRX2) Targeting Moieties

This example compares DiI association and GFP protein expression resulting from KC3 LNPs with different densities of CD8-targeting moieties (15C01 and TRX2) at different dose levels. Nanoparticles bearing GFP encoding mRNA (and a fluorescent dye label (Dil-C18-5DS)) were produced using the microfluidic mixing and buffer exchange processes described in Example 41 above. An αCD8 Fab or VHH conjugate or αCD3 Fab was incorporated into the parent LNPs to obtain the final antibody targeted LNP formulation using the process described in Example 43 above. Particles thus produced were tested in vitro in primary human CD3+ T cells to assess LNP association and reporter protein expression as described in Example 49 above.

At all dose levels, LNPs performed similar between the various targeting moiety densities tested in terms of the fraction of DiI+ T cells (reflected by % DiI+ cells) (FIG. 37A). At the 1 μg/mL dose, DiI MFI values decreased with the increasing 15C01 densities from 5 VHHs per LNP to 20 VHHs per LNP. However, at dose levels <1 μg/mL DiI MFI, values increased with the increasing 15C01 densities (FIG. 37B). At all dose levels, transfection efficiency was increased with the increasing densities of 15C01 in terms of the fraction of GFP+ T cells (reflected by % GFP+ cells) (FIG. 37C). A similar trend was observed in terms of GFP MFI values (FIG. 37D). Expectedly, no GFP expression was observed in CD4+ T cells with CD8-targeted LNPs (15C01 and TRX2) both in terms of % GFP+ and GFP MFI indicating targeting specificity toward CD8+ cells (FIG. 37E and FIG. 37F).

### Example 71. Viability, LNP Association (Measured as DiI-Dye Fluorescence), and GFP Expression in Human Cd8+ T Cells Transfected with Lipid 15 LNPS Containing Different Variants of 15C01

This example compares the viability, Dil association, and GFP protein expression resulting from Lipid 15 LNPs with different optimized variants of the 15C01 &CD8-targeting moiety (e.g., SEQ ID NO: 1, SEQ ID NO: 7, SEQ ID NO: 8, SEQ ID NO: 9). Nanoparticles bearing GFP encoding mRNA (and a fluorescent dye label (DiI-C18-5DS)) were produced using the microfluidic mixing and buffer exchange processes described in Example 41 above. The different variants of 15C01 αCD8 VHH conjugate were incorporated into the parent LNPs to obtain the final antibody-targeted LNP formulation using the process described in Example 43 above. Particles thus produced were tested in vitro in primary human CD8+ T cells to assess reporter gene expression as described in Example 49 above.

All 15C01 targeting variants tested showed a similar level of T cell viability to the untreated control 24 hours post-transfection (FIG. 38A). At all dose levels, the 15C01 variants tested showed comparable levels of LNP association in terms of the fraction of DiI+ T cells (reflected by % DiI+ cells) (FIG. 38B). However, the amount of LNP association per cell (reflected by DiI MFI values) varied between the variants, with the 15C01v8 (SEQ ID NO: 9) variant showing the highest signal at a density of 10 VHHs per LNP at the 1 μg/mL dose (see FIG. 38C and Table 39).

TABLE 39
DiI MFI values of Lipid 15 LNPs targeted with 15C01 variants (SEQ ID NO: 1, SEQ ID NO: 7,
SEQ ID NO: 8, SEQ ID NO: 9) at different densities and treated at various mRNA doses.

| | | | | | | | | | Fold/ | Fold/ | Fold/ |
|---|---|---|---|---|---|---|---|---|---|---|---|
| | | | | | | | | | back- | back- | back- |
| | | | | | | | | | ground | ground | ground |
| | | | | | | | | | (1.0 | (0.3 | (0.06 |
| DiI MFI | 1 (μg/mL) | | 0.3 (μg/mL) | | 0.06 (μg/mL) | | 0 (ug/mL) | | μg/mL) | μg/mL) | μg/mL) |
| V6 (SEQ ID NO: | 27215 | 26637 | 3163 | 3077 | 494 | 317 | | | 723.8 | 83.9 | 10.9 |
| 7) (Density = 2.5) | | | | | | | | | | | |
| V6 (SEQ ID NO: | 40150 | 29944 | 4556 | 3878 | 1038 | 796 | | | 942.1 | 113.4 | 24.7 |
| 7) (Density = 5) | | | | | | | | | | | |
| V6 (SEQ ID NO: | 94371 | 73704 | 18808 | 13296 | 4425 | 2283 | | | 2259.1 | 431.5 | 90.2 |
| 7) (Density = 10) | | | | | | | | | | | |
| V6 (SEQ ID NO: | 135072 | 109337 | 28410 | 20137 | 3547 | 3201 | | | 3285.1 | 652.5 | 90.7 |
| 7) (Density = 15) | | | | | | | | | | | |
| V6 (SEQ ID NO: | 125773 | 135404 | 8392 | 7629 | 4453 | 3823 | | | 3510.4 | 215.3 | 111.2 |
| 7) (Density = 30) | | | | | | | | | | | |
| V7 (SEQ ID NO: | 7237 | 7782 | 1627 | 1801 | 399 | 335 | | | 201.9 | 46.1 | 9.9 |
| 8) (Density = 2.5) | | | | | | | | | | | |
| V7 (SEQ ID NO: | 14618 | 17988 | 3612 | 3663 | 713 | 524 | | | 438.3 | 97.8 | 16.6 |
| 8) (Density = 5) | | | | | | | | | | | |
| V7 (SEQ ID NO: | 51272 | 59904 | 10945 | 11560 | 2860 | 1874 | | | 1494.3 | 302.5 | 63.6 |
| 8) (Density = 10) | | | | | | | | | | | |
| V7 (SEQ ID NO: | 99358 | 119739 | 10318 | 11351 | 2310 | 2091 | | | 2944.9 | 291.3 | 59.2 |
| 8) (Density = 15) | | | | | | | | | | | |
| V7 (SEQ ID NO: | 87469 | 94371 | 14926 | 15171 | 3547 | 3113 | | | 2444.1 | 404.5 | 89.5 |
| 8) (Density = 30) | | | | | | | | | | | |
| V8 (SEQ ID NO: | 19622 | 19948 | 2957 | 3777 | 666 | 534 | | | 531.9 | 90.5 | 16.1 |
| 9) (Density = 2.5) | | | | | | | | | | | |
| V8 (SEQ ID NO: | 31414 | 27739 | 5381 | 4575 | 1152 | 1481 | | | 795.1 | 133.8 | 35.4 |
| 9) (Density = 5) | | | | | | | | | | | |
| V8 (SEQ ID NO: | 146860 | 143996 | 16769 | 21768 | 3208 | 2940 | | | 3909.4 | 518.0 | 82.6 |
| 9) (Density = 10) | | | | | | | | | | | |
| V8 (SEQ ID NO: | 113718 | 139119 | 25337 | 32879 | 4176 | 3619 | | | 3398.3 | 782.5 | 104.8 |
| 9) (Density = 15) | | | | | | | | | | | |
| V8 (SEQ ID NO: | 120329 | 114841 | 33037 | 35167 | 4099 | 4211 | | | 3160.9 | 916.7 | 111.7 |
| 9) (Density = 30) | | | | | | | | | | | |
| 15C01 Parental | 112883 | 120922 | | | | | | | 3142.5 | | |
| Seq. (SEQ ID | | | | | | | | | | | |
| NO: 1) | | | | | | | | | | | |
| (Density = 10) | | | | | | | | | | | |
| mutOKT8 fab | 75.8 | 71.9 | | | | | | | 2.0 | | |
| (Density = 15) | | | | | | | | | | | |
| MBS | | | | | | | 38.5 | 35.9 | | | |

At dose levels <1 μg/mL, the 15C01v8 variant at a density of 30 VHHs per LNP showed increased levels of GFP expression in terms of the fraction of GFP+ T cells (reflected by % GFP+ cells) (FIG. 38D) and in terms of the copy number of GFP molecules produced per cell as reflected by the GFP MFI values (see FIG. 38E and Table 40).

TABLE 40
GFP MFI values of Lipid 15 LNPs targeted with 15C01 variants
at different densities and treated at various mRNA doses.

| | | | | | | | | | Fold/ | Fold/ | Fold/ |
|---|---|---|---|---|---|---|---|---|---|---|---|
| | | | | | | | | | back- | back- | back- |
| | | | | | | | | | ground | ground | ground |
| | | | | | | | | | (1.0 | (0.3 | (0.06 |
| DiI MFI | 1 (ug/mL) | | 0.3 (ug/mL) | | 0.06 (ug/mL) | | 0 (ug/mL) | | ug/mL) | ug/mL) | ug/mL) |
| V6 (SEQ ID NO: | 351 | 344 | 130 | 134 | 74.5 | 69.4 | | | 6.2 | 2.4 | 1.3 |
| 7) (Density = 2.5) | | | | | | | | | | | |
| V6 (SEQ ID NO: | 531 | 471 | 152 | 146 | 92.5 | 88.7 | | | 9.0 | 2.7 | 1.6 |
| 7) (Density = 5) | | | | | | | | | | | |
| V6 (SEQ ID NO: | 1199 | 1042 | 348 | 294 | 149 | 118 | | | 20.1 | 5.7 | 2.4 |
| 7) (Density = 10) | | | | | | | | | | | |
| V6 (SEQ ID NO: | 2136 | 1381 | 562 | 336 | 144 | 109 | | | 31.5 | 8.0 | 2.3 |
| 7) (Density = 15) | | | | | | | | | | | |
| V6 (SEQ ID NO: | 2080 | 2593 | 238 | 249 | 142 | 160 | | | 41.8 | 4.4 | 2.7 |
| 7) (Density = 30) | | | | | | | | | | | |
| V7 (SEQ ID NO: | 142 | 183 | 89.9 | 104 | 59.1 | 69.4 | | | 2.9 | 1.7 | 1.2 |
| 8) (Density = 2.5) | | | | | | | | | | | |
| V7 (SEQ ID NO: | 211 | 281 | 140 | 138 | 82.2 | 77.1 | | | 4.4 | 2.5 | 1.4 |
| 8) (Density = 5) | | | | | | | | | | | |
| V7 (SEQ ID NO: | 770 | 801 | 246 | 237 | 111 | 100 | | | 14.1 | 4.3 | 1.9 |
| 8) (Density = 10) | | | | | | | | | | | |
| V7 (SEQ ID NO: | 1235 | 1305 | 248 | 241 | 103 | 105 | | | 22.7 | 4.4 | 1.9 |
| 8) (Density = 15) | | | | | | | | | | | |
| V7 (SEQ ID NO: | 1475 | 1828 | 376 | 363 | 143 | 135 | | | 29.6 | 6.6 | 2.5 |
| 8) (Density = 30) | | | | | | | | | | | |
| V8 (SEQ ID NO: | 388 | 396 | 149 | 160 | 87.4 | 80.9 | | | 7.0 | 2.8 | 1.5 |
| 9) (Density = 2.5) | | | | | | | | | | | |
| V8 (SEQ ID NO: | 564 | 524 | 186 | 175 | 99 | 107 | | | 9.7 | 3.2 | 1.8 |
| 9) (Density = 5) | | | | | | | | | | | |
| V8 (SEQ ID NO: | 1705 | 1712 | 363 | 387 | 144 | 127 | | | 30.6 | 6.7 | 2.4 |
| 9) (Density = 10) | | | | | | | | | | | |
| V8 (SEQ ID NO: | 1979 | 2123 | 596 | 597 | 162 | 148 | | | 36.7 | 10.7 | 2.8 |
| 9) (Density = 15) | | | | | | | | | | | |
| V8 (SEQ ID NO: | 2568 | 2410 | 862 | 833 | 178 | 177 | | | 44.6 | 15.2 | 3.2 |
| 9) (Density = 30) | | | | | | | | | | | |
| 15C01 Parental | 1895 | 2025 | | | | | | | 35.1 | | |
| Seq. (SEQ ID | | | | | | | | | | | |
| NO: 1) | | | | | | | | | | | |
| (Density = 10) | | | | | | | | | | | |
| mutOKT8 fab | 57.8 | 57.8 | | | | | | | 1.0 | | |
| (Density = 15) | | | | | | | | | | | |
| MBS | | | | | | | 56.5 | 55.2 | | | |

### Example 72. Cd69 Expression in Human Cd8+ T Cells Transfected with Lipid 15 LNPS Containing Anti-CD3 (SP34) and Anti-Cd8 (15C01V8 and TRX2) Targeting Moieties

This example compares the expression levels of the early T cell activation marker, CD69, resulting from Lipid 15 LNPs with αCD8-targeting moieties (TRX2 and 15C01v8) and αCD3-targeting moiety (SP34). Nanoparticles bearing GFP encoding mRNA (and a fluorescent dye label (DiI-C18-5DS)) were produced using the microfluidic mixing and buffer exchange processes described in Example 41 above. αCD8 and αCD3 Fab or VHH conjugate was incorporated into the parent LNPs to obtain the final antibody targeted LNP formulation using the process described in Example 43 above. Particles thus produced were tested in vitro in primary human CD8+ T cells to assess expression levels of CD69 as described in Example 49 and Example 51 above.

Transfection with SP34-targeted LNPs showed elevated levels of CD69 expression compared to antiCD8 (15C01v8 and TRX2) targeting moieties at all dose levels reflected by the CD69 MFI (FIG. 39A, FIG. 39B, and Table 41). This indicates that targeting the CD3 receptor results in T cell activation, which is not a result of targeting CD8.

TABLE 41
GFP MFI values of Lipid 15 LNPs targeted with 15C01v8 variants
at different densities and treated at various mRNA doses.

| CD69 MFI | 3 ug/mL | 1 ug/mL | 0.33 ug/mL | 0.11 ug/mL | 0.037 ug/mL | 0 ug/mL |
|---|---|---|---|---|---|---|
| 15C01v8 | 9143 | 7795 | 4768 | 4216 | 3870 | |
| (SEQ ID 9) | | | | | | |
| mutOKT8 | 4247 | 4416 | 4036 | 4273 | 4078 | |
| SP34 | 78825 | 79433 | 17543 | 8680 | 6574 | |
| TRX2 | 8803 | 10187 | 4732 | 4468 | 4158 | |
| Untreated | | | | | | 3923 |

### Example 73. Mcherry Expression in NHP CD8+ T Cells Transfected with Lipid 15 LNPS Containing Different Anti-CD8 (15C01 or TRX2) Targeting Moieties

This example compares the mCherry protein expression in primary NHP CD8+ T cells resulting from Lipid 15 LNPs with αCD8-targeting moieties (15C01 and TRX2). Nanoparticles bearing mCherry encoding mRNA were produced using the microfluidic mixing and buffer exchange processes described in Example 41 above. Different αCD8-conjugates (15C01 and TRX2) were incorporated into the parent LNPs to obtain the final antibody-targeted LNP formulation using the process described in Example 43 above. Particles thus produced were tested in vitro in primary NHP CD8+ T cells to assess reporter gene expression as described in Example 56 above.

Transfection efficiency was increased with 15C01-targeted LNPs over TRX2-targeted LNPs and the untreated control in terms of the mCherry MFI values (FIG. 40). This indicates that the 15C01 targeting moiety is cross-reactive and can thus bind and be internalized by NHP CD8+ cells.

### Example 74. Illustration of CD19 CAR Cassette Designs

This example shows the designs of the chimeric antigen receptors (CARs) (FIG. 41). Each anti-CD19 binder, in a VHH format, was incorporated into different CAR cassettes containing varying intracellular costimulatory domains. The first CAR cassette was designed to incorporate an intracellular costimulatory domain derived from human CD28 (exemplary CAR Cassette #1). Other CAR cassettes contained intracellular co-stimulatory domains derived from the human 4-1BB domain (exemplary CAR Cassettes #2 and #3). Extracellularly, the CAR cassettes included a CD28 hinge and transmembrane domain and a signaling domain derived from CD3zeta.

### Example 75. Cytokine Secretion of T Cells Transfected with Different Cd19 Car mRNA Constructs by Electroporation

This example compares the level of cytokine secretion (IFN-γ) in primary human CD8+ T cells resulting from anti-CD19 CAR transfection by electroporation. mRNA encoding various anti-CD19 CAR constructs were transfected into primary human CD8+ T cells by electroporation as described in Example 63 above. The level of IFN-γ secreted into the supernatant was assessed by ELISA using the method described in Example 79 below. CD8+ T cells transfected with mRNA encoding clones 40C02 (SEQ ID NO: 710), 36F01 (SEQ ID NO: 719), 44A08 (SEQ ID NO: 707), 36D10 (SEQ ID NO: 720), 46C04 (SEQ ID NO: 699), and 46F07 (SEQ ID NO: 687) incorporated into CAR cassettes showed elevated levels of IFN-Y (>50 μg/mL) in the supernatant in comparison to other CAR constructs tested (see FIG. 42). These data suggest that these constructs mediate undesirable tonic signaling once expressed on the T cell surface.

### Example 76. In Vitro CAR T Cell Function by Cancer Cell Line Co-Culture with ACD19 CAR-Expressing T Cells Derived by Transfection of Primary Human T Cells with ACD8 (15C01) Targeted Lipid 15 LNPS Bearing CAR-mRNA or GFP-mRNA

This example compares cytotoxicity resulting from Lipid 15 LNPs with inserted anti-CD8 (15C01v8 (SEQ ID NO: 9))-targeting moiety encapsulating various anti-CD19 CAR-encoding mRNA payloads. CD8-targeted nanoparticles bearing anti-CD19 CAR-encoding mRNA were produced using the microfluidic mixing and buffer exchange processes described in Example 41 above. An αCD8 VHH conjugate was incorporated into the parent LNPs to obtain the final Nanobody® ISVD-targeted LNP formulation using the process described in Example 43 above. Particles thus produced were tested in vitro in primary human CD8+ T cells co-cultured with target cancer cell lines to assess the CAR-mediated cytotoxicity as described in Example 55 above. The clinically validated anti-CD19 FMC63 clone was used as a benchmark control.

Increased levels of CAR-mediated cytotoxicity against wild-type Nalm6 cells at effector-to-target cell ratios (E:Ts) of 8:1 (FIG. 43A) and 1:1 (FIG. 43B) were observed for CAR constructs incorporating clones αCD19 #7 and αCD19 #6 into CAR cassettes containing a CD28-derived hinge domain of 40 amino acids. The level of cytotoxicity observed was comparable to the clinically validated FMC63 clone. A similar trend was observed against wild-type Raji cells at E:Ts of 8:1 (FIG. 43C) and 1:1 (FIG. 43D). As expected, no cytotoxicity was observed above background, as determined by co-culturing with the non-target expressing K562 cell line E:Ts of 8:1 (FIG. 43E) and 1:1 (FIG. 43F).

### Example 77. Cytotoxicity of Raji Cells Co-Cultured with Human CD8+ T Cells Electroporated with mRNA Encoding Various CD19 CAR Constructs

This example compares the CAR-mediated cytotoxicity of Raji cells resulting from CAR mRNA transfection by electroporation and subsequent co-culture with CD19-expressing Raji cells. mRNA encoding various anti-CD19 CAR constructs were transfected into primary human CD8+ T cells and co-cultured with Raji cells, and then the level of CAR-mediated cytotoxicity was assessed as described in Example 63. The clinically validated FMC63 clone was used as a benchmark control.

Levels of CAR-mediated cytotoxicity (>65%) above the benchmark control was observed in CD8+ T cells transfected with mRNA encoding clones αCD19 #13, αCD19 #16, αCD19 #15, αCD19 #14, αCD19 #12, αCD19 #11, αCD19 #6, αCD19 #7, αCD19 #10, and αCD19 #3 incorporated into CAR cassettes containing a CD28-derived hinge domain of 40 amino acids in length or a CD8alpha-derived hinge domain of 46 amino acids in length (see FIG. 44).

### Example 78. Human IFN-Gamma ELISA Method

IFN-γ levels were assayed using an R&D Duoset IFN-γ ELISA kit, PN DY285B. Briefly: an Immulon 2HB 96-well plate (Thermo X1506319) was coated by adding 100 μL of a 2 μg/mL solution of the R&D IFN-γ capture antibody to each well, and then the plate was incubated overnight at 4° C. The plate was washed three times with wash buffer (0.05 TWEEN-20 in pH 7.4 TRIS buffered saline, Thermo 28360), blocked with reagent diluent (0.1% BSA in wash buffer) for one hour at room temperature, and then washed an additional three times with wash buffer. Supernatants were diluted three-fold in reagent diluent, and then 100 μL of diluted supernatant was added to each well. IFN-γ standards were prepared on the same plate by serial dilution. Plates were incubated for two hours at room temperature and then washed three times with wash buffer. 100 μL of detection antibody diluted in reagent diluent was added and incubated for 2 hours at room temperature, and then the plate was washed three times with wash buffer. 100 μL of Streptavidin-HRP was added and incubated for 20 minutes at room temperature, and then the plate was washed three times with wash buffer. 100 μL of substrate solution (Thermo N301) was added and incubated for 20 minutes at room temperature, and then the reaction was quenched by adding 100 μL of stop solution (Invitrogen SS04). Optical density at 450 nm was read on a SpectraMax M5 plate reader. IFN-γ concentration was quantified relative to a standard curve based on contemporaneously analyzed IFN-γ standards.

### Example 79-1. B Cell Depletion in Human and NHP PBMCS after Transfection Using Lipid 15 LNPS Containing ACD8 (15C01V8) Targeting Moiety and ACD19 CAR mRNA

This example compares the B cell depletion levels in primary human and non-human primate (NHP) PBMCs resulting from Lipid 15 LNPs with an anti-CD8-targeting moiety (15C01v8) and anti-CD19 CAR mRNA. Anti-CD8-targeted nanoparticles bearing anti-CD19 CAR-encoding mRNA were produced using the processes described in Example 41 above. Particles thus produced were tested in vitro in primary human and NHP PBMCs to assess B cell depletion as described in Example 56 above. The level of B cell depletion was assessed by gating on the CD20+ cells and determining the % of dead cells via live/dead stain. B cell depletion was achieved in all species tested using exemplary anti-CD19 VHH CAR constructs (CAR constructs #1-3 and 47-49), indicating NHP cross-reactivity of these anti-CD19 clones (see FIG. 45). For the CAR construct incorporating the clinically validated FMC63 clone, B cell depletion above background was only observed in human PBMCs.

### Example 79-2. B Cell Depletion in Rhesus Macaques after Intravenous Infusion of Lipid 15 LNPs Containing ACD8 (15C01V8) Targeting Moiety and ACD19 CAR mRNA

CD19 CAR expression is associated with a robust decrease in B cells (e.g., as identified via CD20 expression, frequency, and count). Humanized mouse models used herein demonstrated that the CD8-targeted LNP formulation described herein is capable of inducing anti-CD19 CAR expression in CD8+ T cells after repeated dosing. Additional studies in both rhesus macaques and cynomolgus monkeys has shown re-doseablity and resulting B cell depletion.

This example shows that CD8-targeted LNPs could deliver selectively anti-CD19 CAR mRNA into CD8+ T cells in vivo to re-program CD8+ T cells into functional anti-CD19 CAR T cells. B cell depletion was used as a tool to measure functional anti-CD19 CAR T cell persistence. This example compares the B cell depletion levels in an in vivo non-human primate (NHP) model wherein rhesus macaques were intravenously infused with a single dose (SD) or multiple doses (MD) of Lipid 15 LNPs comprising an anti-CD8-targeting moiety (15C01v8) and anti-CD19 CAR mRNA. Anti-CD8-targeted nanoparticles bearing anti-CD19 CAR-encoding mRNA were produced using the processes described in Example 41 above.

Twenty rhesus macaques of approximately 2-4 years of age weighing between 3-6 kg were infused intravenously with 0.1 mg/kg, 0.3 mg/kg, or 1.0 mg/kg as a single dose or multiple doses of CD8-targeted LNPs comprising anti-CD19 CAR mRNA or vehicle control, with 2-4 macaques per dosage group. Intravenous (IV) injection was performed as a single slow infusion (via pump at 5 mL/kg) over a 1-hour duration. Blood, bone marrow, and peripheral lymph node samples were collected for analysis, and macaques were monitored throughout the study for any clinical signs of adverse events. No infusion-related toxicities were observed across any of the groups. Anti-CD19 CAR expression was identified on the surface of CD8+ T cells and NK cells of the spleen (FIG. 69A). Anti-CD19 CAR expression was identified on the surface of CD8+ T cells as early as 1 hr following the intravenous infusion of CD8-targeted LNPs that peaked at 24 hrs after infusion.

A single dose infusion of the CD8-targeted LNPs comprising anti-CD19 CAR mRNA resulted in >95% B cell depletion in peripheral blood of rhesus macaques 24 hrs after treatment relative to the pre-infusion baseline B cell frequencies. Maximal B cell depletion in the peripheral blood was observed at 24 hrs after infusion of the CD8-targeted LNPs, followed by a return to the pre-infusion baseline at 50 days post-infusion. It also was found that CD8+ T cells and NK cells in the bone marrow of the rhesus macaques expressed the anti-CD19 CAR 24 hrs after infusion of the CD8-targeted LNPs. A concomitant decrease of bone marrow resident CD20+ B cells was observed at >90% relative to control animals (FIG. 69B). Similar to peripheral blood, maximal B cell depletion in the bone marrow was observed at 24 hrs after the CD8-targeted LNP infusion and returned to baseline at 29 days post-infusion.

Administration of the CD8-targeted LNP comprising mRNA encoding anti-CD19 CAR #1 to NHPs at 0.1 mg/kg (single dose) or 0.3 mg/kg (single or multi-dose) induced dose-dependent mean reduction of CD19-positive cells (FIG. 70A) and CD20-positive cells (FIG. 70B) in both the spleen and axillary lymph node. This change was significant in the 0.3 mg/kg multi-dose treated group when compared to the vehicle-treated group and was most notable in the spleen. Administration of the CD8-targeted LNP comprising mRNA encoding anti-CD19 CAR #2 to NHPs at 1.0 mg/kg (single) or 0.3 mg/kg (multi-dose) showed reduction of CD19-positive cells (FIG. 70A) and CD20-positive cells (FIG. 70B) in the spleen. This cell depletion was most prominent in the 0.3 mg/kg multi-dose group. In the axillary lymph node, the mean CD19- and CD20-positive cell depletion was comparable between the two dose regimens.

Gross changes in B cell receptor CDR and isotype distribution from B cells harvested from the bone marrow was observed at 57 days post-infusion relative to B cells harvested from the bone marrow pre-infusion. These results demonstrated the in vivo efficacy of the CD8-targeted LNPs comprising anti-CD19 CAR mRNA to target selectively and reprogram CD8+ T cells into functional anti-CD19 CAR T cells.

Staining of NHP samples was performed using the antibodies provided in Table 51 below.

TABLE 51
Staining antibodies for immunophenotyping analysis of NHP samples.

| | | | | | | Target |
|---|---|---|---|---|---|---|
| Marker | Clone | Fluorophore | Vendor | Catalog # | Size | Species |
| Viability | NA | LD Blue | ThermoFisher | L34962 | 1000test | N/A |
| CD3 | SP34-2 | BUV496 | BD | 741183 | 100test | Rhesus |
| CD14 | M5E2 | BUV563 | BD | 741360 | 100test | Rhesus |
| CD4 | L200 | BUV615 | BD | 751365 | 100test | Rhesus |
| CXCR5 | MU5UBEE | BUV737 | ThermoFisher | 367-9185-42 | 100test | Rhesus |
| CD56 | MEM-188 | BUV805 | ThermoFisher | 368-0566-42 | 100test | Rhesus |
| IgD | IA6-2 | super bright 436 | ThermoFisher | 62-9868-42 | 100test | Rhesus |
| CD69 | FN50 | BV510 | BioLegend | 310936 | 100test | Rhesus |
| CD45RA | 5H9 | BV605 | BD | 740424 | 100test | Rhesus |
| CD11c | 3.9 | BV650 | BioLegend | 301638 | 100test | Rhesus |
| CD8 | SK1 | BV711 | BD | 569389 | 100test | Rhesus |
| CD16 | 3G8 | BV750 | BioLegend | 302082 | 100test | Rhesus |
| HLA-DR | L243 | BV785 | BioLegend | 307642 | 100test | Rhesus |
| CD27 | O323 | FITC | BioLegend | 302806 | 100test | Rhesus |
| CD19 | CVID3/429 | AF532 | Novus Biologicals | NBP2-61909AF532 | 100test | Rhesus |
| CD20 | 2H7 | PerCP-Cy5.5 | BD | 560736 | 100test | Rhesus |
| CTLA-4 | 14D3 | PerCP-eF710 | ThermoFisher | 46-1529-42 | 100test | Rhesus |
| CD38 | OKT10 | PE | Leinco Technologies | C547-200ug | 100test | Rhesus |
| CCR7 | G043H7 | PE-Cy7 | BioLegend | 353226 | 100test | Rhesus |
| CD19Ag | N/A | AF647 | Acro | CD9-HA2H6-200ug | 200 ug | Rhesus |
| PD-1 | EH12.2H7 | AF700 | BioLegend | 329952 | 100test | Rhesus |
| CD95 | DX2 | APC-Cy7 | BioLegend | 305636 | 100test | Rhesus |
| (Fas) | | | | | | |

### Example 80. Repeated Car-Mediated Cytotoxicity of NALM6 Cells Co-Cultured With Human Cd8+ T Cells Transfected with Lipid 15 LNPS Containing an Anti-Cd8 (15C01V8) Targeting Moiety and Encapsulating Various Anti-CD19 CAR-Encoding mRNAs In Vitro

This example compares repeated CAR-mediated cytotoxicity of Nalm6 target cells resulting from co-culture with CD8+ T cells after being transfected with Lipid 15 LNPs comprising an anti-CD8-targeting moiety (15C01v8). Anti-CD8-targeted nanoparticles bearing anti-CD19 CAR-encoding mRNA were produced using the processes described in Example 41 above. Particles thus produced were tested in vitro in primary human CD8+ T cells co-cultured with Nalm6 cells to assess CAR-mediated cytotoxicity as described in Example 55 above but with the level of cytotoxicity being assessed at the various time points by utilizing the live cell imaging SX5 Incucyte system. Repeated addition of Nalm6 target cells to the culture was performed every 48 or 72 hours. Anti-CD19 CAR construct #1 showed the highest level of CAR-mediated cytotoxicity upon repeated addition of Nalm6 target cells that was comparable to the CAR incorporating the clinically validated FMC63 clone (see FIG. 46).

### Example 81: Generation of Anti-Cd8 ISVD Molecules Generation of Recombinant Soluble CD8 Proteins

A DNA fragment encoding a secretion signal, the ectodomains of either human or cynomolgous CD8α (CD8alpha) or CD8B (CD8beta), a human IgG1 Fc domain and a HIS tag or Twin-streptag was cloned into a vector suitable for expression in CHOEBNALT85 cells (Icosagen). CHOEBNALT85 cells were transfected with equimolar quantities of both vectors. Both CD8α-Fc homodimers and CD8α/B-Fc heterodimers were purified from the culture broth 7 days after transfection by HIS trap chromatography capturing both CD8α homodimers and CD8α/B heterodimers, followed by Streptactin chromatography, separating CD8α homodimers from CD8α/β heterodimers.

Table 52 depicts the protein sequence of human and cyno CD8α-Fc-HIS6 and CD8β-Fc-Twinstrep chains.

TABLE 52
Human and cyno CD8 sequences for immunization.

| | Amino acid sequence |
|---|---|
| Human CD8α-Fc-HIS6 | SQFRVSPLDRTWNLGETVELKCQVLLSNPTSGCSWLFQPRGAAA |
| (SEQ ID NO: 149) | SPTFLLYLSQNKPKAAEGLDTQRFSGKRLGDTFVLTLSDFRRENE |
| | GYYFCSALSNSIMYFSHFVPVFLPAKPTTTPAPRPPTPAPTIASQP |
| | LSLRPEACRPAAGGAVHTRGLDFACPPCPAPELLGGPSVFLFPPKP |
| | KDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISK |
| | AKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEA |
| | LHNHYTQKSLSLSPGHHHHHH |
| Human CD8β-Fc-Twinstrep | LQQTPAYIKVQTNKMVMLSCEAKISLSNMRIYWLRQRQAPSSDS |
| (SEQ ID NO: 153) | HHEFLALWDSAKGTIHGEEVEQEKIAVFRDASRFILNLTSVKPED |
| | SGIYFCMIVGSPELTFGKGTQLSVVDFLPTTAQPTKKSTLKKRVC |
| | RLPRPETQKGPLCPPCPAPELLGGPSVFLFPPKPKDTLMISRTPEVTC |
| | VVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPG |
| | WSHPQFEKGGGSGGGSGGSSAWSHPQFEK |
| Cynomolgous CD8α-Fc-HIS6 | NQFRVSPLGRTWNLGETVELKCQVLLSNPTSGCSWLFQPRGTA |
| (SEQ ID NO: 155) | ARPTFLLYLSQNKPKAAEGLDTQRFSGKRLGDTFVLTLRDFRQE |
| | NEGYYFCSALSNSIMYFSHFVPVFLPAKPTTTPAPRPPTPAPTTAS |
| | QPLSLRPEACRPAAGGSVNTRGLDFACPPCPAPELLGGPSVFLFPP |
| | KPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHE |
| | ALHNHYTQKSLSLSPGHHHHHH |
| Cynomolgous CD8β-Fc- | LQQTPAYIQVQTNKMAMLSCEAKISLGNMRIYWLRQRQAPSSDS |
| Twinstrep | HHEFLALWDSTKGTVHGEEVAQEKIAVFRDASRFILNLTSVKPE |
| (SEQ ID NO: 157) | DSGIYFCMIIGSPELTFGKGTQLSVVDFLPTTAQPTKKSTPKKRG |
| | CRLPRPATQKGPLCPPCPAPELLGGPSVFLFPPKPKDTLMISRTPEVT |
| | CVVVDVSHEDPEVKFNWYVDGVEVHNAKTKPREEQYNSTYRVVSV |
| | LTVLHQDWLNGKEYKCKVSNKALPAPIEKTISKAKGQPREPQVYTL |
| | PPSRDELTKNQVSLTCLVKGFYPSDIAVEWESNGQPENNYKTTPPVL |
| | DSDGSFFLYSKLTVDKSRWQQGNVFSCSVMHEALHNHYTQKSLSLS |
| | PGWSHPQFEKGGGSGGGSGGSSAWSHPQFEK |

### Immunizations

To induce a heavy-chain antibody (HcAb)-dependent humoral immune response, two llamas and one alpaca were immunized four times with DNA encoding full-length recombinant human CD8α and CD8β.

### Library Constructions and Phage Display

Immune blood samples were taken at regular intervals, serum responses were determined, and total RNA was prepared from the isolated PBL. From these blood samples, peripheral blood mononuclear cells (PBMCs) were prepared using Ficoll-Hypaque according to the manufacturer's instructions (Amersham Biosciences, Piscataway, NJ, US). From the PBMCs, total RNA was extracted and used as starting material for RT-PCR to amplify the ISVD-encoding DNA segments, essentially as described in WO 2005/044858. Subsequently, phages were prepared according to standard protocols (see for example the prior art and applications filed by Ablynx N.V. cited herein) and stored after filter sterilization at 4° C. for further use.

For general phage display selection techniques, reference is made to Antibody Phage Display: Methods and Protocols (First Edition, 2002, O'Brian and Aitken eds., Humana Press, Totowa, NJ). Two rounds of panning phage display selections were performed using the llama and alpaca ISVD immune libraries in alternating selection rounds on human and cynomolgus CD8α-Fc homodimers or CD8α/β heterodimers. The output from the selection was plated onto LB/amp/2% glu plates. Colonies were picked and grown in 96 deep well plates (1 ml volume) and induced by adding IPTG for ISVD expression. Periplasmic extracts (volume: ~80 μl) were prepared according to standard methods (see for example the prior art and applications filed by applicant cited herein).

### Primary Screening and Sequencing

Primary screening of periplasmic extracts was performed by a bead based multiplex screening assay (xMAP). Human CD8α-Fc, human CD8α/β-Fc, cyno CD8α-Fc or cyno CD8α/β Fc proteins were coupled to spectrally distinct sets of Magplex beads, a mix of the respective beads was prepared and incubated with 1/10 diluted periplasmic extract and anti-FLAG-PE as a detection reagent. Microspheres coupled with huIgG were additionally included in the mix so that clones that bind to the Fc portion of the CD8-Fc proteins could be excluded.

Clones that showed binding to human and cyno CD8α-Fc, but not to huIgG were sequenced Sequence analysis of ISVDs from phage display selection outputs was done according to commonly known procedures (Pardon et al. 2014, Nat. Protoc. 9:674). 731 unique sequences were obtained.

### Example 82: Characterization of the Unique Sequences

### Off-Rate Analysis of the Unique ISVD Clones

All unique clones from the sequence analysis were subjected to off-rate analysis towards human and cyno CD8α-Fc and CD8α/β-Fc by Surface Plasmon Resonance (SPR) on a ProteOn XPR36 instrument (Bio-Rad Laboratories). The experiment was performed at 25° C. As running buffer 2×HBS-EP+ was used. Anti-hu-Fc ISVD was immobilized on 6 different ligand lanes from a GLC sensorchip (Bio-Rad Laboratories) with the ProteOn Amine Coupling Kit (Bio-Rad Laboratories) according to the manufacturer's instructions. Human CD8α-Fc, cyno CD8α-Fc, human CD8α/β-Fc and cyno CD8α/β-Fc were captured on the anti-hu-Fc ISVD immobilized ligand lanes. One ligand lane served as a reference surface and no target was captured on the anti-hu-Fc ISVD surface. Samples (periplasmic extracts containing unique ISVDs) diluted 1:10 in running buffer were flowed over for 2 minutes, followed by a constant flow of the assay buffer for 10 minutes. Between sample injections, the surfaces were regenerated with 0.85% H₃PO₄. Several buffer blanks were injected for double referencing.

Data was analyzed with the ProteOn Manager 3.1.0 software (Bio-Rad Laboratories), off-rates were determined based on the 1:1 interaction model (Langmuir binding model). Only clones with an off rate slower than 10-2/s were retained.

### Selection of Initial Lead Panel

Based on off-rate on human and cyno CD8 and on sequence uniqueness, 43 clones were selected for expression, purification, and characterization.

### Example 83: Generation of ISVD Constructs

For the selected clones, ISVD-containing DNA fragments, obtained by PCR with specific combinations of forward FR1 and reverse FR4 primers each carrying a unique restriction site, were digested with the appropriate restriction enzymes, and ligated into the matching cloning cassettes of ISVD expression vectors (described below). The ligation mixtures were then transformed to electrocompetent Escherichia coli TG1 (60502, Lucigen, Middleton, WI) or TOP10 (C404052, ThermoFisher Scientific, Waltham, MA) cells which were then grown under the appropriate antibiotic selection pressure (kanamycin or Zeocin). Resistant clones were verified by Sanger sequencing of plasmid DNA (LGC Genomics, Berlin, Germany).

ISVDs were expressed in E. coli TG1 from a plasmid expression vector containing the lac promoter, a resistance gene for kanamycin, an E. coli replication origin and a ISVD cloning site preceded by the coding sequence for the OmpA signal peptide. In frame with the ISVD coding sequence, the vector codes for a C-terminal FLAG3 and HIS6 tag. The signal peptide directs the expressed ISVD to the periplasmic compartment of the bacterial host.

HIS6-tagged ISVDs were purified by immobilized metal affinity chromatography (IMAC) followed by a desalting step and if necessary, gel filtration chromatography (Superdex column, GE Healthcare) in PBS. Else, ISVDs were purified on Protein A followed by a desalting step and if necessary, gel filtration chromatography (Superdex column, GE Healthcare) in PBS. The purity and integrity of the ISVDs was verified by SDS-PAGE and mass spectrometry.

### Example 84: Characterization in Cell-Based Assay

The monovalent lead panel ISVDs were characterized in binding FACS.

### Binding FACS

Binding of purified anti-CD8 ISVDs to CD8⁺ T cells was evaluated by flow cytometry. A serial dilution of each ISVD starting at 1 μM was applied to human T cells (purified from peripheral blood monocytes). ISVDs were allowed to bind for 30 minutes at 4° C. in FACS buffer (PBS supplemented with 10% FBS and 0.05% azide). Cells were washed by centrifugation/resuspension in FACS buffer and bound ISVD probed with a Brilliant Violet 421 anti-Flag antibody (Biolegend; cat #: F1804) for 30 minutes at 4° C. In the same incubation step, CD4+ T cells were probed with APC anti-human CD4 [RPA-T4] (Biolegend). Cells were washed again and incubated with propidium Iodide to stain for dead cells. The cells were analysed via an iQue Plus. Dead cells and CD4+ cells were gated out from the analysis (mean fluorescence intensity in function of ISVD concentration).

For 22 of the 43 ISVDs, a dose-response curve could be fitted and an EC50 calculated (Table 53 below).

TABLE 53
FACS binding analysis of purified ISVDs on human CD8+ T cells.

| | FACS (EC50) (M) |
|---|---|
| ID | CD8⁺ T cells |
| T0347013F01 | 1.5E−10 |
| T0347014D02 | 1.8E−10 |
| T0347014G05 | 1.9E−10 |
| T0347015C01 | 2.0E−10 |
| T0347023A12 | 4.2E−10 |
| T0347002C04 | 4.6E−10 |
| T0347022A02 | 4.9E−10 |
| T0347014D12 | 8.4E−10 |
| T0347014D12 | 1.0E−09 |
| T0347001B04 | 1.4E−09 |
| T0347001E05 | 2.0E−09 |
| T0347001B02 | 2.3E−09 |
| T0347021B10 | 2.5E−09 |
| T0347013B01 | 2.6E−09 |
| T0347002C07 | 4.3E−09 |
| T0347002A07 | 7.3E−09 |
| T0347014A02 | 8.3E−09 |
| T0347012C08 | 8.5E−09 |
| T0347001D12 | 1.1E−08 |
| T0347013G07 | 1.4E−08 |
| T0347008C01 | 2.6E−08 |
| T0347008A10 | 2.8E−08 |

### Functional Testing of Monovalent ISVDs in T Cell Transfection Assays

See above Examples.

The functional assay allowed a clear ranking of the initial lead panel. Clones from different families that had reasonable potency in the TT assay and FACS were selected. This led to a filtered lead panel of 4 ISVDs.

### Affinity Determination of Monovalent ISVDs of Filtered Lead Panel

The affinity (K_{D}) of the lead panel clones to human and cyno CD8α-Fc was determined via SPR on a Biacore 8K⁺ (Cytiva). The experiment was performed at 25° C., as running buffer HBS-EP+1× (Cytiva, cat #BR100669) was used. An anti-hIgG(Fc) ISVD was immobilized on a Biacore CM5 chip by amine coupling according to the manufacturer's instructions. Human and cynomolgus CD8α-Fc were captured via their Fc-portion at a concentration of 1.25 μg/ml, and then nine different concentrations of the clones (diluted in assay buffer) were applied. Samples were applied to the respective targets in multi-cycle kinetics for 2 minutes, followed by a constant flow of the running buffer for 15 minutes. Between the different injections, the surfaces were regenerated with 0.85% H₃PO₄. Data was analysed with the Biacore Insight Evaluation software V5.0.18.22102 (Cytiva). The kinetic properties (ka and kd) were determined using the Langmuir 1:1 model. The equilibrium dissociation constant K_{D} was calculated as the kd/ka ratio. Results are shown in Table 54.

TABLE 54
Kinetic analysis of lead panel ISVDs on human and cynomolgus CD8α-Fc.

| | | Ka | Kd | K_{D} |
|---|---|---|---|---|
| ID | Target | 1/Ms | 1/s | M |
| T0347015C01 | Human CD8α-Fc | 2.27E+06 | 3.01E−04 | 1.33E−10 |
| (SEQ ID NO: 1) | Cynomolgus CD8α-Fc | 2.58E+06 | 2.72E−04 | 1.06E−10 |
| T0347001E05 | Human CD8α-Fc | 4.79E+06 | 4.53E−04 | 9.47E−11 |
| | Cynomolgus CD8α-Fc | 2.66E+05 | 1.18E−03 | 4.45E−09 |
| T0347002A07 | Human CD8α-Fc | 2.82E+06 | 2.73E−02 | 9.67E−09 |
| | Cynomolgus CD8α-Fc | 9.43E+04 | 2.02E−02 | 2.14E−07 |
| T0347013G07 | Human CD8α-Fc | 2.07E+06 | 2.52E−02 | 1.22E−08 |
| | Cynomolgus CD8α-Fc | 5.72E+04 | 1.05E−04 | 1.84E−09 |

Based on the affinity determination and on all other characterization data, ISVD T0347015C01 (SEQ ID NO: 1) was selected for further optimization.

### Example 85: Sequence Optimization of ISVD T0347015C01

The protein sequence of anti-CD8 ISVD T0347015C01 was further optimized, involving the mutagenesis of one or more specific amino acid residues to make the ISVD more humanlike, to reduce its binding by pre-existing antibodies and to improve its long-term stability. Nine sequence variants of ISVD T0347015C01 (SEQ ID NOs: 1-9) showed preserved binding (ka, kd and KD) to human and cynomolgus CD8α-Fc (Tables 55-56). Their melting temperature (TSA) was determined, and their functional properties as part of a targeted lipo nanoparticle were assessed.

TABLE 55
Kinetic analysis of ISVD T0347015C01 variants on human
and cynomolgous CD8α-Fc.

| | | Ka | Kd | KD | Tm |
|---|---|---|---|---|---|
| ID | Target | 1/Ms | 1/s | M | ° C. |
| A044300805 | Human CD8a-Fc | 2.11E+06 | 4.97E−04 | 2.35E−10 | |
| (SEQ ID NO: 1) | Cynomolgus CD8a-Fc | 1.77E+06 | 5.37E−04 | 3.03E−10 | 74 |
| A044300805_v1 | Human CD8a-Fc | 1.18E+06 | 6.06E−04 | 5.12E−10 | |
| (SEQ ID NO: 2) | Cynomolgus CD8a-Fc | 1.11E+06 | 6.89E−04 | 6.20E−10 | 73 |
| A044300805_v2 | Human CD8a-Fc | 2.41E+06 | 5.54E−04 | 2.30E−10 | |
| (SEQ ID NO: 3) | Cynomolgus CD8a-Fc | 1.88E+06 | 5.64E−04 | 3.00E−10 | 74 |
| A044300805_v3 | Human CD8a-Fc | 2.69E+06 | 5.09E−04 | 1.89E−10 | |
| (SEQ ID NO: 4) | Cynomolgus CD8a-Fc | 1.84E+06 | 4.98E−04 | 2.70E−10 | 70 |
| A044300805_v4 | Human CD8a-Fc | 1.34E+06 | 5.21E−04 | 3.89E−10 | |
| (SEQ ID NO: 5) | Cynomolgus CD8a-Fc | 1.22E+06 | 6.22E−04 | 5.09E−10 | 69 |
| A044300805_v5 | Human CD8a-Fc | 3.38E+06 | 5.99E−04 | 1.77E−10 | |
| (SEQ ID NO: 6) | Cynomolgus CD8a-Fc | 2.48E+06 | 5.96E−04 | 2.40E−10 | 70 |
| A044300805_v6 | Human CD8a-Fc | 1.93E+06 | 4.87E−04 | 2.52E−10 | |
| (SEQ ID NO: 7) | Cynomolgus CD8a-Fc | 1.63E+06 | 4.90E−04 | 3.02E−10 | 74 |
| A044300805_v7 | Human CD8a-Fc | 1.51E+06 | 5.97E−04 | 3.95E−10 | |
| (SEQ ID NO: 8) | Cynomolgus CD8a-Fc | 1.29E+06 | 6.47E−04 | 5.00E−10 | 68 |
| A044300805_v8 | Human CD8a-Fc | 2.77E+06 | 5.35E−04 | 1.93E−10 | |
| (SEQ ID NO: 9) | Cynomolgus CD8a-Fc | 3.71E+06 | 7.51E−04 | 2.02E−10 | 69 |

TABLE 56
ISVD T0347015C01 variants on human and cynomolgous CD8α-Fc.

| name | Description (modifications compared to T0347015C01) |
|---|---|
| Abm CDR | |
| definition | |
| T0347015C01 | T0347015C01 |
| A044300805 | T0347015C01(L11V, A14P, D30E, G55E, N56Q, I60A, S71R, K83R, A89L, |
| | N100kG, M100oL, K105Q) |
| A044300805 v1 | T0347015C01(L11V, A14P, D30E, G55E, N56Q, I60A, S71R, K83R, A89L, |
| (T0347015C01v1; | K100bE, N100KG, M100oL, K105Q) |
| or 15C01v1) | |
| A044300805 v2 | T0347015C01(L11V, A14P, D30E, G55E, N56Q, I60A, S71R, K83R, A89L, |
| (T0347015C01v2; | K100bY, N100kG, M100oL, K105Q) |
| or 15C01v2) | |
| A044300805 v3 | T0347015C01(L11V, A14P, D30E, G55E, N56Q, I60A, S71R, Y79D, K83R, |
| (T0347015C01v3; | A89L, N100KG, M100oL, K105Q) |
| or 15C01v3) | |
| A044300805 v4 | T0347015C01(L11V, A14P, D30E, G55E, N56Q, I60A, S71R, Y79D, K83R, |
| (T0347015C01v4; | A89L, K100bE, N100KG, M100oL, K105Q) |
| or 15C01v4) | |
| A044300805 v5 | T0347015C01(L11V, A14P, D30E, G55E, N56Q, I60A, S71R, Y79D, K83R, |
| (T0347015C01v5; | A89L, K100bY, N100KG, M100oL, K105Q) |
| or 15C01v5) | |
| A044300805 v6 | T0347015C01(L11V, A14P, D30E, G55E, N56Q, I60A, S71R, Y79S, K83R, |
| (T0347015C01v6; | A89L, N100KG, M100oL, K105Q) |
| or 15C01v6) | |
| A044300805 v7 | T0347015C01(L11V, A14P, D30E, G55E, N56Q, I60A, S71R, Y79S, K83R, |
| (T0347015C01v7; | A89L, K100bE, N100KG, M100oL, K105Q) |
| or 15C01v7) | |
| A044300805 v8 | T0347015C01(L11V, A14P, D30E, G55E, N56Q, I60A, S71R, Y79S, K83R, |
| (T0347015C01v8; | A89L, K100bY, N100KG, M100oL, K105Q) |
| or 15C01v8) | |
| Kabat CDR | |
| definition | |
| T0347015C01 | T0347015C01 |
| A044300805 | T0347015C01(L11V, A14P, D30E, G55E, N56Q, I60A, S71R, K83R, A89L, |
| | N100kG, M100oL, K105Q) |
| A044300805 v1 | T0347015C01(L11V, A14P, D30E, G55E, N56Q, I60A, S71R, K83R, A89L, |
| (T0347015C01v1; | K100bE, N100kG, M100oL, K105Q) |
| or 15C01v1) | |
| A044300805 v2 | T0347015C01(L11V, A14P, D30E, G55E, N56Q, I60A, S71R, K83R, A89L, |
| (T0347015C01v2; | K100bY, N100kG, M100oL, K105Q) |
| or 15C01v2) | |
| A044300805 v3 | T0347015C01(L11V, A14P, D30E, G55E, N56Q, I60A, S71R, Y79D, K83R, |
| (T0347015C01v3; | A89L, N100kG, M100oL, K105Q) |
| or 15C01v3) | |
| A044300805 v4 | T0347015C01(L11V, A14P, D30E, G55E, N56Q, I60A, S71R, Y79D, K83R, |
| (T0347015C01v4; | A89L, K100bE, N100KG, M100oL, K105Q) |
| or 15C01v4) | |
| A044300805 v5 | T0347015C01(L11V, A14P, D30E, G55E, N56Q, I60A, S71R, Y79D, K83R, |
| (T0347015C01v5; | A89L, K100bY, N100KG, M100oL, K105Q) |
| or 15C01v5) | |
| A044300805 v6 | T0347015C01(L11V, A14P, D30E, G55E, N56Q, I60A, S71R, Y79S, K83R, |
| (T0347015C01v6; | A89L, N100KG, M100oL, K105Q) |
| or 15C01v6) | |
| A044300805 v7 | T0347015C01(L11V, A14P, D30E, G55E, N56Q, I60A, S71R, Y79S, K83R, |
| (T0347015C01v7; | A89L, K100bE, N100kG, M100oL, K105Q) |
| or 15C01v7) | |
| A044300805 v8 | T0347015C01(L11V, A14P, D30E, G55E, N56Q, I60A, S71R, Y79S, K83R, |
| (T0347015C01v8; | A89L, K100bY, N100KG, M100oL, K105Q) |
| or 15C01v8) | |

### Melting Temperature of T0347015C01 Sequence Variants

A thermal shift assay (TSA) was performed in a 96-well plate on a qPCR machine (LightCycler 480II, Roche). Per row, one ISVD protein was analyzed in the following pH range: 4, 5, 6, 7, 8 and 9.

Per well, 5 μL of ISVD protein sample (0.8 mg/mL in D-PBS) was added to 5 μL of Sypro Orange (40× in MilliQ water; Invitrogen, Cat. No. S6551) and 10 μL of buffer (100 mM phosphate, 100 mM borate, 100 mM citrate and 115 mM NaCl with a pH ranging from 4 to 9). A temperature gradient (37 to 99° C. at a rate of 0.03° C./s) was applied, which induced unfolding of the ISVD proteins, and hence exposure of hydrophobic patches. Binding of Sypro Orange to those hydrophobic patches, caused increase in fluorescence intensity, which was measured (Ex/Em=465/580 nm). The inflection point of the first derivative of the fluorescence intensity curve at pH 7 served as a measure of the melting temperature (Tm).

The Tm (° C.) values obtained for the sequence optimized variants are given in Table 57.

TABLE 57
Tm of ISVD T0347015C01 sequence variants.

| ID | Tm (° C.) |
|---|---|
| A044300805 | 74 |
| A044300805_v1 | 73 |
| A044300805_v2 | 74 |
| A044300805_v3 | 70 |
| A044300805_v4 | 69 |
| A044300805_v5 | 70 |
| A044300805_v6 | 74 |
| A044300805_v7 | 68 |
| A044300805_v8 | 69 |

### Example 86: Preparation of a DSPE-PEG3.4K-15C01 Conjugate

86.1. Expression of 15C01 with GCC Tag

For the expression of GCC tagged VHH15C01 in P. pastoris, the yeast expression vectors contain the AOX1 promoter and terminator, a resistance gene for Zeocin and the coding information for the Saccharomyces cerevisiae α-mating factor signal peptide was used. P. pastoris cells (NRRL Y-11430 cells; ATCC 76273) containing the 15C01-GGC (amino acid sequence of SEQ ID NO:28) encoding vector construct were grown in glycerol fed batches and induction was initiated by the addition of methanol. Standard fed batch fermentations conditions were used.

### 86.2. Capture Step

The anti-CD8 VHH was first isolated from the harvest stream using the standard affinity chromatography on protein A resin. The product eluting from this affinity chromatography step was a mixture of anti-CD8 VHH monomers and dimers. The amount of dimers was quantified by size exclusion HPLC (SE-HPLC). 58% of VHH was a dimer.

### 86.3. Addition of One Reduction Step Before the Polish Step Increases Conjugation Yield

In this example, the influence of a reduction step before the polish step on the conjugation yield was tested. Two experiments were conducted, one with and one without a reduction step before the polish step.

86.3.1 Experiment without Reduction Step Before the Polish Step

In a first experiment, the product obtained in the capture step was immediately purified by anion exchange chromatography (AEX) on Capto Q ImpRes (from Cytiva). After equilibration in 25 mM Tris pH7.5, the product (i.e., mixture of monomer and dimer) adjusted at pH 7.5±0.2 was loaded on the column. After washing the column with the same 25 mM Tris pH7.5 buffer, the product was eluted with a salt gradient from 0 to 2000 mM of NaCl. FIG. 47 shows the chromatographic profile obtained. The AEX chromatographic profile showed two peaks corresponding to the monomeric and the dimeric fractions. The monomers elute first from the column, followed by the dimers.

In this first experiment the dimeric fraction, i.e., the second peak eluting from the AEX chromatography, was further processed. The dimeric product was reduced using a final concentration of TCEP of 5 mM, for 90 min at room temperature under stirring. After reduction, TCEP was removed by UF/DF with a cut off of 10 kDa with seven diafiltration buffer volumes of PBS pH 7.4 with 5 mM EDTA. Analysis of the product by SE-HPLC showed complete reduction of the product, where monomers represented more than 98%.

Conjugation was done by addition of the lipid mixture containing DSPE-PEG2k and DSPE-PEG3.4k-Malemeide. The molar ratio of ‘DSPE-PEG2k:DSPE-PEG3.4k-Malemeide’ was ‘1:4’. Product concentration was first adjusted to 2 mg/mL with PBS pH 7.4, 5 mM EDTA and heated at the temperature of 37° C. The required volume of lipid mix was then added in order to reach the molar ratio ‘VHH:DSPE-PEG2k:DSPE-PEG3.4k-Malemeide’ of ‘1:1:4’. After an incubation of 2 hrs under stirring, the conjugation was stopped by addition of 15 mM of free Cysteine with 5 mM of EDTA for 15 min at 37° C.

Finally, a second UF/DF with a cut off of 100 kDa was done on the conjugated product. The goal of this final step was to remove unconjugated free anti-CD8 VHH that flows through the UF/DF membrane, when the conjugated VHH in micellar form is retained. During this UF/DF 100 kDa, the buffer was exchanged to 25 mM HEPES pH 7.4, 150 mM NaCl. The conjugation yield determined by UV was 66%.

86.3.2 Experiment with a Reduction Step Before the Polish Step

In a second experiment, the affinity protein A chromatography eluate was submitted to a first reduction step with 5 mM of TCEP for 1.5 h at room temperature under stirring. After reduction, the product was immediately purified by AEX using the same protocol as described for the first experiment (see 87.3.1). The chromatogram (FIG. 48) showed a single peak corresponding to the monomeric fraction obtained after reduction. The eluate from the AEX contained 99% of monomer.

Following AEX, the product was reduced with 0.1 mM TCEP for 90 min at room temperature and under stirring. TCEP was then removed from the product with UF/DF 10 kDa and conjugated using the same protocol as described for the first experiment (see 87.3.1). The unconjugated product was removed by UF/DF 100 kDa as explained for the first experiment (see 87.3.1). The UV measurement showed an improved conjugation yield of 74%.

86.4 Optimization of the Reduction Step after the Polish Step

In this example, the second reduction step, after polish and prior to conjugation, was optimized to increase the conjugation yield.

Prior to the conjugation step, the C-terminal cysteine of the GGC tag needs to be free for conjugation to the maleimide functional group of the lipid nanoparticle (LNP). Hence a reduction is needed after the polish chromatography to convert formed VHH dimers to monomers. As the VHH scaffold also contains internal disfulphide bonds, a balance must be struck between sufficient reduction and avoiding over-reduction of these canonical disulphide bonds in the VHH scaffold which could lead to potential over conjugation.

This reduction step after the polish step was optimized using the Design of Experiments (DoE) concept. The experimental design varied 4 factors:
1. pH and buffer system:
   a. 25 mM L-Histidine pH 6.0
   b. 25 mM BIS-TRIS pH 6.5
   c. 25 mM TRIS pH 7.5
2. TCEP concentration:
   a. 1× molar excess to Nb (i.e. equimolar quantity)
   b. 10× molar excess to Nb
   c. 20× molar excess to Nb
3. Conjugation incubation time:
   a. 1 h
   b. 2 h
   c. 16 h
   d. 20 h
4. EDTA concentration:
   a. No EDTA
   b. 5 mM EDTA

The full experimental design is displayed in Table 58.

TABLE 58
Experimental design of the optimization of the reduction step after the polish step.
Four factors (conjugation incubation time, buffer system and pH, EDTA concentration, and
TCEP concentration) were tested in a DoE experimental set up.

| | Incubation time | | | TCEP |
|---|---|---|---|---|
| Experiment | [hours] | Buffer pH | EDTA [mM] | [molar excess to Nb] |
| 1 | 2 | 7.5 | 0 | 20 |
| 2 | 1 | 7.5 | 5 | 1 |
| 3 | 20 | 7.5 | 0 | 1 |
| 4 | 2 | 6 | 0 | 20 |
| 5 | 1 | 6.5 | 0 | 20 |
| 6 | 20 | 6.5 | 0 | 10 |
| 7 | 20 | 6 | 0 | 1 |
| 8 | 1 | 6 | 0 | 1 |
| 9 | 2 | 6.5 | 0 | 10 |
| 10 | 16 | 7.5 | 5 | 1 |
| 11 | 2 | 6.5 | 0 | 1 |
| 12 | 16 | 6 | 0 | 1 |
| 13 | 1 | 7.5 | 0 | 10 |
| 14 | 1 | 6.5 | 5 | 1 |
| 15 | 20 | 7.5 | 0 | 20 |
| 16 | 16 | 6 | 5 | 20 |
| 17 | 2 | 7.5 | 0 | 1 |
| 18 | 2 | 6 | 5 | 1 |
| 19 | 20 | 6.5 | 5 | 20 |
| 20 | 20 | 6 | 5 | 1 |
| 21 | 20 | 6 | 0 | 20 |
| 22 | 2 | 6.5 | 5 | 20 |
| 23 | 1 | 6 | 5 | 20 |
| 24 | 16 | 7.5 | 5 | 20 |
| 25 | 16 | 6.5 | 0 | 20 |
| 26 | 16 | 7.5 | 0 | 10 |
| 27 | 20 | 7.5 | 5 | 10 |
| 28 | 16 | 6.5 | 5 | 10 |
| 29 | 1 | 7.5 | 5 | 20 |
| 30 | 2 | 7.5 | 5 | 10 |

To evaluate the experiments, three assays were applied to all samples. The first analytical technique was SE-HPLC under non-reducing conditions to evaluate the reduction efficiency by determining the ratio of monomer and dimer VHH. The second analytical technique was RP-(U)HPLC under non-reducing conditions which can quantify the amount of missing canonical disulphide bridges due to overreduction. The last assay was to verify whether there was limited residual TCEP present after stopping the reduction reaction, as this could alter the monomer-dimer ratio before analysis.

The protocol was started with dimerized VHH at a concentration of 2.2 mg/mL. The material was buffer exchanged to their respective pH and buffer system each containing 230 mM NaCl. EDTA was added from a 500 mM stock solution to some samples according to the experimental design. Reduction reaction was initiated by adding the appropriate amount of TCEP from a 400 mM stock solution. Incubation of the 1.6 mL total reaction volume was done at room temperature under mixing conditions in a Thermoshaker at 300 RPM. The reduction was stopped by removing the TCEP from the VHH using a 4 kDa MWCO 10 mL Zeba Spin Desalting Column to buffer exchanging it to D-PBS containing 5 mM EDTA. Samples for residual TCEP analysis were taken after this buffer exchange. The bulk of the reduced material was subsequently capped by adding 2-Iodoacetamide (IAA) in highly purified water from a 375 mM freshly made stock solution to a final concentration of 17.85 mM. The capping fixates the monomer-dimer ratio and prevents further over conjugation before analysis on SE-HPLC and RP-(U)HPLC. Results are demonstrated in Table 59.

TABLE 59
Results of the DoE experiment to optimize reduction process after polish step.

| | | | TCEP | | | Residual |
|---|---|---|---|---|---|---|
| | Time | EDTA | (molar | SE-HPLC | RP-(U)HPLC | TCEP |
| Buffer pH | (h) | (mM) | excess) | (% monomer) | (% missing S-S) | (mM) |
| His pH 6.0 | 1 | 0 | 1 | 6.61 | 1.44 | <QL |
| His pH 6.0 | 2 | 5 | 1 | 15.75 | 2.02 | <QL |
| His pH 6.0 | 16 | 0 | 1 | 70.43 | 3.58 | <QL |
| His pH 6.0 | 20 | 0 | 1 | 42.07 | 3.46 | <QL |
| His pH 6.0 | 20 | 5 | 1 | 47.12 | 3.28 | <QL |
| His pH 6.0 | 1 | 5 | 20 | 29.25 | 2.79 | 0.009 |
| His pH 6.0 | 2 | 0 | 20 | 57.00 | 3.30 | <QL |
| His pH 6.0 | 16 | 5 | 20 | 89.15 | 3.76 | 0.066 |
| His pH 6.0 | 20 | 0 | 20 | 94.93 | 3.59 | 0.073 |
| BIS-TRIS pH 6.5 | 1 | 5 | 1 | 22.24 | 2.51 | <QL |
| BIS-TRIS pH 6.5 | 2 | 0 | 1 | 20.63 | 2.11 | <QL |
| BIS-TRIS pH 6.5 | 2 | 0 | 10 | 26.77 | 2.64 | <QL |
| BIS-TRIS pH 6.5 | 16 | 5 | 10 | 98.35 | 3.90 | 0.039 |
| BIS-TRIS pH 6.5 | 20 | 0 | 10 | 88.05 | 3.78 | 0.013 |
| BIS-TRIS pH 6.5 | 1 | 0 | 20 | 31.23 | 2.97 | <QL |
| BIS-TRIS pH 6.5 | 2 | 5 | 20 | 73.30 | 3.86 | 0.030 |
| BIS-TRIS pH 6.5 | 16 | 0 | 20 | 98.21 | 3.91 | 0.063 |
| BIS-TRIS pH 6.5 | 20 | 5 | 20 | 98.58 | 4.18 | 0.055 |
| TRIS pH 7.5 | 1 | 5 | 1 | 31.72 | 2.85 | <QL |
| TRIS pH 7.5 | 2 | 0 | 1 | 49.87 | 3.68 | <QL |
| TRIS pH 7. 5 | 16 | 5 | 1 | 90.91 | 3.31 | <QL |
| TRIS pH 7.5 | 20 | 0 | 1 | 84.60 | 3.51 | <QL |
| TRIS pH 7.5 | 1 | 0 | 10 | 45.22 | 2.07 | <QL |
| TRIS pH 7.5 | 2 | 5 | 10 | 85.36 | 3.67 | 0.023 |
| TRIS pH 7.5 | 16 | 0 | 10 | 98.84 | 4.10 | 0.021 |
| TRIS pH 7.5 | 20 | 5 | 10 | 99.03 | 3.94 | 0.011 |
| TRIS pH 7.5 | 1 | 5 | 20 | 52.78 | 4.45 | 0.017 |
| TRIS pH 7.5 | 2 | 0 | 20 | 84.07 | 3.99 | 0.105 |
| TRIS pH 7.5 | 16 | 5 | 20 | 99.01 | 4.68 | 0.050 |
| TRIS pH 7.5 | 20 | 0 | 20 | 98.83 | 4.42 | 0.024 |

The relative peak area related to over conjugation on RP-(U)HPLC analysis showed little variation as all experiments resulted in an acceptable over conjugation between 1.44% and 4.68%. There were no restrictions on the process parameters in the design space based on the over conjugation risk.

The removal of TCEP to stop the reduction reaction was confirmed by the low TCEP quantification with the highest concentration being 0.105 mM.

The buffer system and pH played a role in an effective reduction. The higher the pH, the faster and thus the better was the reduction. The same applies for the TCEP concentration, higher amounts of TCEP corresponded to a faster and more robust reduction. For incubation times of 1 h and 2 h timepoint, there was a high variability ranging from 31.72% monomer to 85.36% at pH 7.5. While the reduction was much more robust at pH 7.5 for timepoints between 16 and 20 hours, reflected by a narrower range of monomer ranging from 84.60% to 99.03%.

The optimal process parameters for the second reduction (after the polish step) were defined as 25 mM TRIS pH 7.5, combined with a reduction time between 16-20 hours and 20 times molar excess of TCEP for robustness. 2.5 mM EDTA was still added as a pre-caution to prevent oxidation potentially leading to dimerization.

### 86.5 Removal of TCEP

### 86.5.1 Removal of TCEP is Necessary Before Conjugation

In this example, the influence of TCEP on the conjugation of the anti-CD8 VHH with the DSPE-PEG-3.4k-Malemeide was investigated. Purified anti-CD8 VHH was reduced with increasing concentrations of TCEP for 1.5 hour at room temperature. Following the reduction and buffer exchange, the anti-CD8 VHH was conjugated with a lipid mix of DSPE-PEG-2k and DSPE-PEG-3.4k-Malemeide in order to form the anti-CD8 VHH-DSPE-PEG-3.4k conjugate in micellar form. Conjugation was done at 37° C. for 2 h under agitation. Table 60 below summarizes the results obtained.

TABLE 60
Levels of monomeric/dimeric anti-CD8 VHH after TCEP reduction and its influence
on the conjugation of the anti-CD8 VHH with DSPE-PEG-3.4k-Malemeide.

| | | | Concentration of | Level of | Level of |
|---|---|---|---|---|---|
| TCEP | Level of monomer | Level of dimer | TCEP at beginning | unconjugated | conjugated anti- |
| concentration | after reduction | after reduction | of conjugation | ant-CD8 VHH | CD8 VHH |
| (mM) | % | % | mM | % | % |
| 0.01 | 73.2 | 26.8 | <0.01 | 38 | 62 |
| 1.4 | 99.0 | 1.0 | 0.87 | 77 | 23 |
| 2.8 | 99.5 | 0.5 | 1.09 | 80 | 20 |

Increasing the concentration of TCEP improves the reduction of dimers to monomers. At the lower concentration of 0.01 mM TCEP, 73% of the product was monomeric. At higher concentration of 1.4 and 2.8 mM, at least 99% of the product was monomeric.

Following the reduction step, part of the TCEP was removed by chromatography. The concentration of residual TCEP after chromatography and before start of conjugation was lower than 0.01 mM when 0.01M TCEP was used for the reduction step and 0.87 and 1.09 mM when higher concentrations of TCEP had been used for the conjugation step. This higher of amount of residual TCEP negatively impacted the conjugation reaction analysed by Reverse Phase-HPLC connected to a CAD detector. In absence of TCEP (<0.01 mM), 62% of the anti-CD8 VHH was conjugated, and 38% did not react with the lipids. From this 38% of unreacted product, 27% was a dimer that had no free Cysteine for conjugation. On the other hand, at higher TCEP concentration with more than 99% of monomer free for conjugation, only approximately 20% of the anti-CD8 VHH conjugated with the lipids while 80% of free product did not react with the DSPE-PEG-3.4k-Malemeide. These data demonstrate that complete removal of TCEP is necessary for the successful conjugation of the anti-CD8 VHH with the DSPE-PEG-3.4k Maleimide.

86.5.2 Buffering the VHH in PBS Avoids Dimerization of the VHH

Stability of the monomeric anti-CD8 VHH in the absence of TCEP was evaluated in this example. In a first experiment, removal of TCEP with UF/DF 10 kDa was demonstrated. The concentration of TCEP was measured in the permeate sample at different timepoints of the UF/DF (Table 61).

TABLE 61
Concentration of TCEP during the UF/DF 10 kDa.

| Sample | Level of TCEP (mM) |
|---|---|
| Before the start of Reduction | <0.01 |
| End of Reduction | 0.045 |
| Diafiltration Volume = 1 | 0.032 |
| Diafiltration Volume = 2 | 0.014 |
| Diafiltration Volume = 3 | <0.01 |
| Diafiltration Volume = 4 | <0.01 |
| Diafiltration Volume = 5 | <0.01 |
| Final UFDF pool after 5 Diafiltration volumes | <0.01 |

After 3 diafiltration volumes, a complete clearance of TCEP was observed.

In a second experiment, reduced anti-CD8 VHH was submitted to UF/DF 10 kDa. During this UF/DF, TCEP was completely removed and the anti-CD8 VHH was buffered in 2.68 mM KCl, 1.47 mM KH₂PO₄, 136.9 mM NaCl, 8.10 mM Na₂HPO₄ pH 7.4 with 2.5 mM of EDTA. This buffered product without any TCEP was put on hold for 14 days at different temperatures without agitation and the stability of the monomeric form (in the absence of TCEP) was measured at different time points (Table 62).

TABLE 62
Percentage of monomeric anti-CD8 VHH without
TCEP in phosphate buffer at different temperatures,
measured at different time intervals.

| Temperature [° C.] | Time [days] | Level of monomer [%] |
|---|---|---|
| Start material for all | 0 | 98.5 |
| three temperatures | | |
| Room temperature | 1 | 97.9 |
| Room temperature | 3 | 96.8 |
| Room temperature | 14 | 93.2 |
| 2-8 | 1 | 98.4 |
| 2-8 | 3 | 97.7 |
| 2-8 | 14 | 96.6 |
| ≤−20 | 3 | 97.4 |
| ≤−20 | 14 | 96.8 |

Results in Table 62 show that the level of monomers remained above 96% for 3 days at the three temperatures. For longer hold times, where samples are normally stored at low temperatures of 2-8° C. or frozen at −20° C., the monomeric form remained above 96% for 14 days. These results show the stability of the anti CD8-VHH in the absence of TCEP in the selected buffer.

### 86.6 Conjugation Step

In this example, the conjugation step was optimized to increase the conjugation yield and increase the number of VHH molecules on the PEG-Micelle. As described above, before the conjugation, the anti-CD8 VHH polish eluate material was submitted to a reduction step followed by an UF/DF step to remove TCEP. Then, the anti-CD8 VHH was prepared for the conjugation step. Conjugation was based on the reaction of a lipid mixture containing DSPE-PEG2k-Methoxy and DSPE-PEG3.4k-Maleimide. DSPE-PEG3.4k-Maleimide is the reagent containing maleimide which is the functional moiety that drives the conjugation reaction. As a result of the conjugation step, a lipid micelle is obtained with anti-CD8 VHH conjugated to it. The conjugation step can be evaluated by means of the conjugation recovery (i.e., how many molecules of VHH have efficiently reacted) and by the DSPE-PEG2k-Methoxy: VHH ratio. This ratio, evaluated after UF/DF II, describes the micelle composition. It indicates the amount of VHH molecules that decorates the PEG-lipid micelle. The smallest this ratio is, the more VHH molecules decorate the PEG-lipid micelle, hence presenting more antiCD8 recognition sites.

The conjugation step was optimized using the Design of Experiments (DoE) concept. Two main goals were defined: a) to increase conjugation recovery; and b) to decrease the DSPE-PEG2k-Methoxy: VHH ratio.

The experimental design varied 7 factors:
1. pH and buffer system:
   a. 10 mM Sodium phosphate pH 7.5
   b. 10 mM Sodium citrate pH 6.5
2. NaCl concentration:
   a. 0 mM
   b. 70 mM
   c. 180 mM
   d. 250 mM
3. Ratio VHH:DSPE-PEG3.4k-Maleimide
   a. 1
   b. 2
   c. 3
   d. 4
4. Ratio DSPE-PEG3.4k-Maleimide:DSPE-PEG2k-Methoxy
   a. 1.5
   b. 4
5. VHH concentration
   a. 0.5 mg/mL
   b. 3 mg/mL
   c. 6 mg/mL
   d. 10 mg/mL
6. Incubation step
   a. 2 h
   b. O/N (16-20 h)
7. Temperature
   a. RT
   b. 37° C.

The full experimental design was performed in 30 runs (conditions). To evaluate the experiments, SE-HPLC and RPC chromatography followed by UV/CAD detector were applied to the different samples. SE-HPLC under non-reducing conditions evaluated the reduction efficiency to confirm that a suboptimal reduction step resulting in a high dimer content was not impacting the conjugation outcome. RPC-UV followed the protein part of the conjugation whereas CAD detector studied the lipidic part.

The protocol started with anti-CD8 VHH material that had been submitted to the second reduction step. To remove the reducing agent, the reduced material was buffer exchanged with Zeba Spin Desalting Column 7K MWCO to their respective pH and buffer system. Protein concentration, pH, buffer system and NaCl concentration were adapted before conjugation for each condition. 5 mM EDTA was also added to each condition. Samples for analysis by SE-HPLC and RPC-UV under non-reducing conditions were taken after the buffer exchange. Before analysing, these samples were capped by adding 2-Iodoacetamide (IAA) in highly purified water from a 375 mM freshly made stock solution to a final concentration of 17.85 mM. The capping fixates the monomer-dimer ratio and prevents further over conjugation before analysis on SE-HPLC and RP-(U)HPLC.

The conjugation reaction was performed with two different PEG-Lipids: DSPE-3.4 kDa PEG-Maleimide and DSPE-2 kDa PEG-Methoxy. Initially, each lipid was resuspended separately to a 3.4 mM concentration in 1 mM citric acid pH 5.7 to generate two separate stock solutions.

For each condition, and based on factor #4 (Ratio DSPE-PEG2k:DSPE-PEG3.4-Maleimide) values, the needed volume from the two separate stock solutions were mixed together. The appropriate volume of PEG-Lipid mix, following the defined ratio VHH:DSPE-PEG3.4-Maleimide in Factor #3, was added to 1.6 mL of the VHH sample. The VHHs presented different concentration values, based on Factor #5. This preparation was mixed at 400 rpm in a ThermoMixer equipment and incubated at 21° C. (2 hrs or O/N) or 37° C. (2 hrs or O/N). Overnight conditions never exceeded 20 hrs.

After the incubation time, the conjugation reaction was stopped by quenching it with L-Cysteine. The appropriate volume of a stock solution of 15 mM L-Cysteine+5 mM EDTA in D-PBS was added to achieve a final concentration of 1.5 mM L-Cysteine. Samples were mixed at 37° C. for 15 minutes while shaking at 400 rpm in Thermomixer during quenching step. After the incubation time, the samples for RPC-UV and RPC-CAD were taken.

The samples after the reduction step and subsequent buffer exchange presented a monomer percentage higher than 93% measured by non-reduced SE-HPLC. This confirms that the reduction step was sufficient to proceed with the conjugation. The reduction step also did not generate missing disulphide brides based on RP-(U)HPLC results.

RPC-UV values from quenched conjugated samples showed a relative peak area of conjugated anti-CD8 VHH with a variation between 21% and 70%. Free anti-CD8 VHH (i.e., anti-CD8 VHH molecules that have not been conjugated) presented a peak area with a wide variation, with values from 26% to 85%. Peak area related to over conjugation presented limited variation with values ranging from 2% to 8%.

Information on the DSPE-PEG2k-Methoxy: VHH ratio in the samples was obtained by combining RPC-UV and RPC-CAD data. The highest value that was obtained in the DoE was 24 and the lowest one was 1.

Over conjugation evolution was also evaluated. Over conjugation is undesirable, and any conditions that would favor it should be discarded.

To increase the conjugation recovery, there were two key parameters: the VHH concentration and the ratio VHH:DSPE-PEG3.4-Maleimide. The conjugation recovery response (i.e., percentage of conjugate VHH) followed an inverted-U-shaped curve for both parameters. Regarding the VHH concentration, the maximum conjugation recovery was achieved when the conjugation was performed between 3-4.5 g/L. Regarding the ratio VHH:DSPE-PEG3.4-Maleimide, the maximum conjugation recovery was achieved with a ratio of 2.8. With higher ratios, from 3 onwards, not only the conjugation recovery decreased but also the over conjugation increased.

The ratio of the conjugation reagents impacted the micelle composition. The ratio DSPE-PEG2k-Methoxy:VHH ratio decreased almost in a linear way when the ratio VHH:DSPE-PEG3.4k-Maleimide also decreased or when using smaller DSPE-PEG3.4k-Maleimide: DSPE-PEG2k-Methoxy ratios. A variation in DSPE-PEG3.4k-Maleimide: DSPE-PEG2k-Methoxy ratio did not impact the over conjugation neither the conjugation recovery. However, a decrease in VHH: DSPE-PEG3.4k-Maleimide, from 2.3 downwards, presented a negative impact on conjugation recovery, going down until 50%.

The presence or absence of NaCl, buffer system, pH selected, incubation time and temperatures were factors that showed no impact on the conjugation recovery or micelle composition based on DoE results.

Based on the different results obtained, we defined the optimal process conditions for conjugation. The conjugation reagents are preferably added to a VHH solution in D-PBS+2.5 mM EDTA pH 7.4 with a protein concentration between 5.0-7.0 g/L (before conjugation), 3-4.5 g/L during conjugation. The ratio of the reagents is preferably 1 mol VHH:2 mol DSPE-PEG3.4k-Maleimide: 3 mol DSPE-PEG2k-Methoxy. The conjugation reaction is preferably stirred at 20-22° C. during 105-135 minutes. Afterwards, the conjugation reaction is preferably stopped by quenching it with L-Cysteine. The appropriate volume of a stock solution of 15 mM L-Cysteine+5 mM EDTA in D-PBS is preferably added to achieve a final concentration of 1.5 mM L-Cysteine. The sample is preferably mixed at 37° C. for 15 minutes while shaking at 400 rpm in Thermomixer.

### Example 87. In Vitro Transcription (IVT)

In vitro transcription reaction is using enzymatic reaction transcribing mRNA from DNA template.

Reaction mixture contains DNA template, ribo-nucleotides, enzyme, RNase inhibitor and corresponding buffer. DNA template must contain polymerase promoter. DNA template can be either linearized plasmid DNA, or PCR product, or annealed complimentary oligonucleotides.

Typical IVT protocol:
1. Linearize plasmid DNA template, ensuring that linearization site is at the desired 3′end of mRNA to be synthesized.
2. Purify linearized DNA.
3. Mix reaction mixture and incubate at 35-55° C. for 1-3 hrs.
4. DNAse treatment of the reaction mixture to remove DNA template.
5. Purify mRNA from the reaction mixture.

Example using T7 polymerase.

This reaction produces 100-200 μg of mRNA using 1 μg DNA template.
1. Assemble the reaction at room temperature in the following order:

| REAGENT | AMOUNT | FINAL CONCENTRATION |
|---|---|---|
| Nuclease-free water | X μl | |
| 10X Reaction Buffer | 2 μl | |
| ATP (100 mM) | 2 μl | 10 mM final |
| GTP (100 mM) | 2 μl | 10 mM final |
| UTP (100 mM) | 2 μl | 10 mM final |
| CTP (100 mM) | 2 μl | 10 mM final |
| Template DNA | X μl | 1 μg |
| T7 RNA Polymerase Mix | 2 μl | |
| Total Reaction Volume | 20 μl | |

2. Mix thoroughly, pulse-spin in microfuge. Incubate at 37° C. for 2 hours.
3. DNase treatment to remove DNA template. Add 70 μl nuclease-free water, 10 μl of 10× DNase I Buffer, and 2 μl of DNase I (RNase-free), mix and incubate for 15 minutes at 37° C.
4. Proceed with purification of synthesized RNA or analysis of transcription products by gel electrophoresis.

Resulting mRNA will have 5′-triphosphate and can be enzymatically capped.

It is also possible to generate co-trascriptionally capped mRNA. In this case reaction should include cap analog (e.g., CleanCap), and reaction mixture should be assembled as following:

| | | FINAL |
|---|---|---|
| REAGENT | AMOUNT | CONCENTRATION |
| Nuclease-free water | X μl | |
| 10X Reaction Buffer | 2 μl | |
| ATP (100 mM) | 2 μl | 10 mM final |
| UTP (100 mM) | 2 | 10 mM final |
| CTP (100 mM) | 2 μl | 0 mM final |
| GTP (20 mM) | 2 μl | 2 mM final |
| Cap Analog (40 mM) | 4 μl | 8 mM final |
| Template DNA | X μl | 1 μg |
| T7 RNA Polymerase Mix | 2 μl | |
| Total Reaction Volume | 20 μl | |

### Example 88. Additional Methodology Details

### LNP Transfection in Human Whole Blood

After collection from eight healthy human donors into sodium heparin collection tubes (BD Biosciences), 100 μL fresh human whole blood was seeded into a 96-well round bottom plate and transfected with targeted LNPs. Red blood cells were lysed 24 hours post-transfection using Versalyse lysis buffer (Beckman Coulter) and immune cells were Fc blocked and stained with CD14-BUV495 (BD Biosciences), CD8-BUV661 (BD Biosciences), CD16-BV421 (BD Biosciences), CD4-BV605 (BD Biosciences), CD3-BV711 (BD Biosciences), CD19-BV785 (Biolegend), CD56-PE (Biolegend), and CD15-PE/Cy5 (Biolegend). Dead cells were excluded from analysis by using eFluor780 fixable viability dye (Thermo Fisher). Granulocytes were gated based on SSC and CD16. Total lymphocytes were gated based on SSC and the following subsets were defined: CD4+ T cells were defined as CD3+, CD56−, and CD4+; CD8+ T cells were defined as CD3+, CD56−, and CD4− due to steric hindrance and internalization mediated by the CD8-targeted LNPs; NKT cells were defined as CD3+ and CD56+; NK cells were defined as CD3− and CD56+; and B cells were defined as CD19+ or CD20+. The GFP expression was evaluated after 24 hours of transfection in all the abovementioned cell subsets. The gating strategy is shown in FIG. 49.

### Incucyte Cytotoxicity Assay

The Incucyte NucLight Red Lentivirus reagent (Sartorius) was used to transduce Nalm6 cells following the manufacturer's protocol. Transduced NucLight Red positive cells were selected using puromycin, and >95% purity was confirmed by flow cytometry prior to co-culture assays. CD8+ T cells were isolated and transfected as described above. Unbound LNPs were removed by washing in PBS 2 hours post-transfection. Transfected T cells were then co-cultured with NucLight Red Lentivirus-transduced Nalm6 cells in T cell media (RPMI-1640, 10% FBS and 50 ng/mL recombinant human IL-2) in a 96-well flat-bottom plate. Cancer cell killing was monitored in the SX5 Incucyte every 2 hours. Re-challenge of the T cells was performed by gently removing the supernatant and replacing it with NucLight Red Lentivirus-transduced Nalm6 cells in T cell media. Re-transfection was performed by gently removing the supernatant and replacing it with T cell media containing fresh LNPs. The level of cytotoxicity was quantified by normalizing the count of red cells to the initial time point.

### Optimized ICL and PEG-Lipid Improves the T-Cell Transfection Efficiency

With the ionizable lipid component playing a key role in the transfection efficiency, different ionizable lipid families were designed and tested that were formulated into LNPs using microfluidic mixing (FIG. 54A). A traceable Dil dye was incorporated into the LNPs comprising reporter mRNA encoding eGFP to evaluate the efficiency with which the formulated LNPs could get to and deliver a functional mRNA payload to human T-cells. The DiI-LNPs encapsulating GFP mRNA were inserted with an anti-CD8 or anti-CD3 antibody conjugate post-formulation and added to primary human T-cells to evaluate the LNP-association and GFP expression by Flow Cytometry and to compare the different ionizable lipid families (FIGS. 54B-54E). Initial development of the platform utilized previously identified ICLs (FIGS. 56A-56F) such as KC2-DMA, ALC-0315, SM-102, KC3-DMA, or a dimethylaminopropyl-dialkyl lipid (TID-02) either as components of the investigational LNPs or as part of comparator LNPs to demonstrate differentiation from LNPs including a novel ICL series shown in FIGS. 57A-57C.

Molecular design features of this novel lipid family included the use of an aminopropyl diol linker group to bridge the dimethyl aminopropyl ionizable head group to two hydrophobic lipid “tail” groups via degradable ester linkages and a single less hydrophobic N-acyl amide linked “side” group. The two “tail” groups and the single “side” group were chosen to impart an asymmetric structure to the lipid molecule, and the resulting cone-shaped structure was additionally amplified using either degree of unsaturation or by incorporation of branched aliphatic substituents. Furthermore, degradable ester linkages were introduced systematically into both “side” and “tail” groups to impart in vivo biodegradability and enable rapid lipid elimination.

In lipids 1 through 8, the N-acyl “side” group consists of 8 carbon (Lipid 5), 9 carbon (Lipids 2, 3 and 4), 10 carbon (Lipids 1 and 8), or 11 carbon (Lipids 6 and 7)O-acyl substituents in combination with either dioleoyl (lipid 4) or di-linoleoyl (Lipids 1, 2, 3, 5 & 6, 7, 8)O-acyl “tail” groups, thereby spanning a range of “side” group hydrophobic content with mono- and di-unsaturated “tail” groups. Similarly, the three-dimensional structure and steric properties of the “side” were tuned via position and degree of branching, i.e., using linear and branched (Lipid 2 versus Lipids 3 and 4), or α- and β-substitution (Lipid 6 versus 8) of the alkyl groups within the N-acyl “side” group.

In lipids 9, 10, 15, 16, 24A, and 26, a succinate-derived degradable ester linkage was introduced into the “side” group, and the hydrophobicity of the ester moiety was controlled via the choice of succinate ester composition, i.e., less hydrophobic 3-octanol succinate (in Lipid 16) or more hydrophobic 3-decanol succinate (in Lipids 9, 10, 15, 24A and 26). Also, in this sub-set, the lipid “tail” groups comprised of di-linoleoyl (Lipids 9 and 10), di-oleoyl (Lipids 15, 16), di-(8-hydroxydecanoate) (Lipid 24A) or 4-((2-hexyldecanoyl)oxy) butanoate (Lipid 26)O-acyl substituents. These tail group compositions were chosen to evaluate the effect of the degree of lipid unsaturation (Lipids 9, 10, 15, and 16) and branching (Lipids 24A and 26) as these structural parameters are expected to modulate molecular shape (“cone” structure) and lipid melting points and thereby alter the ability of the resulting LNP formulations to enable efficient endosomal membrane disruption and cytosolic delivery of the RNA payload.

Lipid 15 showed superior transfection efficiency, as illustrated by increased GFP MFI values (FIG. 54D), in comparison to other ionizable lipid families that were tested, including the ionizable lipid, ALC-0315, which was used in the SARS-COV-2 vaccine, BNT162b2. Additionally, Lipid 15 LNPs proved to be stable after one freeze-thaw cycle as no significant decrease in T cell transfection performance was observed. This contrasted with LNPs based on ALC-0315, where a significant drop in T cell transfection efficiency was observed after a single freeze-thaw cycle. See, e.g., FIGS. 58A-66E and Tables 63-70). A single olefin was preferred over side-chains with dual olefins (e.g. linoleic acid; Lipids 8-10). Biodegradable ester linkages and N-acyl substituents derived from succinate-linked branched aliphatics resulted in improved LNP colloidal stability. Lipids that displayed a strongly positive charge at acidic pH (endosomal) and neutral or slightly anionic at neutral physiologic pH were preferable with regard to activity and selectivity.

Optimization of the PEG-lipid component is critical for the delivery and potency of targeted LNPs., PEG-lipid conjugates with varying anchor lengths di-C14 (DMG-PEG), di-C16 (DPG-PEG), and di-C18 (DSG-PEG), and backbone chemistries (diacylglycerol versus dialkylphosphatidylethanolamine) were compared (structures shown in FIGS. 57B and 57C). To optimize the PEG component of the formulation, CD3 Fab-targeted LNP formulations were compared based on the ionizable lipid KC2 containing 2.5% DMG-PEG, DPG-PEG, and DSG-PEG, respectively, in vivo.

### An Anti-CD8 Nanobody® Facilitates Specific Uptake in Human T Cells

Using the selected Lipid 15, the transfection efficiency of LNPs inserted with various targeting moieties against different T cell targets was evaluated. Fabs against CD3 and CD8, respectively, as well as a Nanobody® ISVD against CD8 (Nb8) were tested, and it was found that increased transfection efficiency at various doses and time points could be achieved using the CD8-targeting Nanobody® ISVD (see FIGS. 55A-55C and FIGS. 67A, 67B, and 67E). Additionally, CD8-mediated transfection did not result in elevated release of IFN-γ (FIG. 55D) and TNF-α (FIG. 67F), nor upregulation of the early T cell activation marker, CD69 (FIGS. 67C and 67D), as opposed to transfection using CD3-targeted LNPs.

Following the selection of Nb8 as the lead targeting moiety, the amino acid sequence was further optimized to make the binder more human-like, to reduce its binding by pre-existing antibodies, and to improve its long-term stability. The resulting optimized variants were conjugated, inserted into LNPs, and compared by transfecting T cells in vitro. Nb8-H3 showed superior binding and GFP expression compared to other variants tested and was thus selected as the targeting moiety for subsequent experiments (FIGS. 68A-68E).

Having observed high transfection efficiency in isolated T cells, next was sought to investigate the level of delivery specificity. An ex vivo assay was developed, testing human whole blood from 8 healthy donors to evaluate the expression efficiency in various cell subsets by Flow Cytometry (FIG. 55E). Significant off-target cell transfection in the Granulocyte and B cell populations was not observed (FIG. 55E). Additionally, the CD8-targeted LNPs showed high transfection efficiency in the CD8⁺ cell subsets, including CD8⁺ T cells, NKT cells, and NK cells, while no significant GFP expression was observed in the CD4⁺ T cell population. Generally, modest differences in transfection efficiencies between the donors tested were observed, but all 8 donors showed a high degree of cell specificity, as no GFP expression could be observed in off-target cell populations.

### LNP Characterization

The LNPs were characterized to determine the average hydrodynamic diameter, zeta potential, and mRNA content (total and dye-accessible). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer.

RNA content of the nanoparticles was measured using Thermo Fisher Quant-iT RiboGreen RNA Assay Kit. Dye-accessible RNA, which includes both non-incorporated RNA and RNA that is near the surface of the nanoparticle, was measured by diluting the nanoparticles to approximately 1 μg/mL mRNA using HEPES buffered saline, and then adding Quant-iT reagent to the mixture. Total RNA content was measured by diluting the particles to 1 μg/mL mRNA using HEPES buffered saline, disrupting the nanoparticles by heating them to 60° C. for 30 minutes in HEPES buffered saline containing 0.5% Triton, and then adding Quant-It reagent. RNA was quantified by measuring fluorescence at 485/535 nm, and concentration was determined relative to a contemporaneously run RNA standard curve.

### Cryogenic Electron Microscopy (Cryo-EM)

Cryo Transmission Electron Microscopy was performed by NanoImaging Services (San Diego, CA). Samples were prepared by applying a 3 μL drop of sample suspension to a cleaned grid, blotting with filter paper, and then vitrifying the sample in liquid ethane. Electron microscopy was performed using a Thermo Fisher Scientific Glacios Cryo Transmission Electron Microscope (Cryo-TEM) operated at 200 kV and equipped with a Falcon 4 direct electron detector. Images were acquired at a nominal underfocus of −4.1 μm to −2.0 μm and electron doses of ~11-21 e⁻/Å².

Novel Ionizable Cationic Lipid Development for T Cell Reprogramming with Targeted LNPs

A novel ionizable lipid family (of the general structure shown in FIG. 57A) was screened in targeted LNP formulations to identify the optimal molecular features for reprogramming CD8-positive (CD8+) T cells. Lipid selection criteria included the physiochemical properties of the resulting LNPs as well as the ability of the resulting formulations to illicit reporter protein expression in an in vitro primary human CD8+ T cell transfection experiment using αCD8-targeted LNPs. See, e.g., FIGS. 57A-57C.

Molecular design features of this novel lipid family included the use of an aminopropyl diol linker group to bridge the dimethyl aminopropyl ionizable head group to two hydrophobic lipid “tail” groups via degradable ester linkages and a single less hydrophobic N-acyl amide linked “side” group. The two “tail” groups and the single “side” group were chosen to impart an asymmetric structure to lipid molecule, and the resulting cone-shaped structure was additionally amplified using either degree of unsaturation or by incorporation of branched aliphatic substituents. Furthermore, degradable ester linkages were introduced systematically into both “side” and “tail” groups to impart in vivo biodegradability and enable rapid lipid elimination. As seen in Scheme S1, in lipids 1 through 8.

N-acyl “side” group consists of 8 carbon (Lipid 5), 9 carbon (Lipids 2, 3 and 4), 10 carbon (Lipids 1 and 8), or 11 carbon (Lipids 6 and 7) O-acyl substituents in combination with either dioleoyl (lipid 4) or di-linoleoyl (Lipids 1, 2, 3, 5 & 6, 7, 8) O-acyl “tail” groups, thereby spanning a range of “side” group hydrophobic content with mono- and di-unsaturated “tail” groups. Similarly, the three-dimensional structure and steric properties of the “side” were tuned via position and degree of branching, i.e., using linear and branched (Lipid 2 versus Lipids 3 and 4), or α- and β-substitution (Lipid 6 versus 8) of the alkyl groups within the N-acyl “side” group.

In lipids 9, 10, 15, 16, 24A, and 26, a succinate-derived degradable ester linkage was introduced into the “side” group, and the hydrophobicity of the ester moiety was controlled via the choice of succinate ester composition, i.e., a less hydrophobic 3-octanol succinate (in Lipid 16) or more hydrophobic 3-decanol succinate (in Lipids 9, 10, 15, 24A and 26). Also, in this sub-set, the lipid “tail” groups comprised of di-linoleoyl (Lipids 9 and 10), di-oleoyl (Lipids 15, 16), di-(8-hydroxydecanoate) (Lipid 24A) or 4-((2-hexyldecanoyl)oxy) butanoate (Lipid 26)O-acyl substituents. These tail group compositions were chosen to evaluate the effect of the degree of lipid unsaturation (Lipids 9, 10, 15, and 16) and branching (Lipids 24A and 26) as these structural parameters are expected to modulate molecular shape (“cone” structure) and lipid melting points and thereby alter the ability of the resulting LNP formulations to enable efficient endosomal membrane disruption and cytosolic delivery of the RNA payload.

### Physiochemical Properties of Lipids 1-11 and Lipid 15 Targeted LNPs

Lipids 1, 2, 3, 4, 5, 6, 7, 8, 9, 10, 11, and 15 LNPs encapsulating GFP-mRNA (TriLink Biotechnologies Inc.) were prepared and characterized as described (see Methods Section). As seen in FIGS. 58A and 59A, LNP diameter increased incrementally upon buffer exchange from HBS (pH 7.4) into MBS (pH 6.5), as well as upon insertion of antibody conjugate (via 4 hr incubation at 37° C.) and after 1× freeze/thaw cycle. The magnitude of the upward shift in LNP diameter was lipid dependent suggesting a relationship between LNP colloidal stability and ionizable lipid chemistry. Lipid 2, 3, and 8 LNPs exhibited only small changes in particle diameter during the processing and storage steps, suggesting that nine and ten carbon (C9 and C10)N-acyl substitution constitutes the most optimal head group composition in lipids based on di-linoleoyl O-acyl lipid tails. Additionally, comparing lipids 8 to 1 (both C10 N-acyl substituted), the β-ethyl substitution (to the N-acyl group in lipid 8) resulted in better size stability relative to α-ethyl substitution (in lipid 1). Both smaller (less hydrophobic, e.g., C8 N-acyl substitution of lipid 5) and larger (more hydrophobic, i.e., C11 N-acyl substitution of lipids 6 and 7) resulted in a larger shift in LNP size indicating loss of LNP colloidal stability relative to the C9 and C10 analogs. C11 N-acyl group (lipids 6 and 7) also exhibited a lower zeta potential value at pH 5.5 relative to all other lipids consistent with lower LNP apparent pKa value (Tables 63 and 64). This downward shift in LNP apparent pKa is attributed to more pronounced neighboring group effects with increasing hydrophobicity of the N-acyl substituent. Furthermore, the optimum head group chemistry was dependent on lipid tail group composition. A nine-carbon (C9)N-acyl substituent in lipid 4 (bearing oleoyl lipid tail groups) exhibited more significant LNP size shifts relative to size changes observed in lipids 2 and 3 LNPs (bearing linoleoyl tail groups). See, e.g., FIGS. 58A-58B and FIGS. 59A-59B.

TABLE 63
LNP charge (Zeta Potential) of Lipids 1-8. Charge
(Zeta Potential) (DLS, mV) in pH 5.5 MES and pH 7.4 HBS
of LNPs with Lipids 1-8 and comparator Lipids SM-102,
ALC-0315, MC3, and KC3, respectively.

| | LNP charge | LNP charge |
|---|---|---|
| Ionizable Lipid, LNP no. | (ZP, mV); pH 5.5 | (ZP, mV); pH 7.4 |
| Lipid 1, DPG-PEG | 19.9 | −0.212 |
| Lipid 2, DMG-PEG | 25.3 | 3.64 |
| Lipid 2, DPG-PEG | 23.1 | 2.03 |
| Lipid 3, DMG-PEG | 21.7 | 1.18 |
| Lipid 3, DPG-PEG | 19.6 | −2.01 |
| Lipid 4, DPG-PEG | 21.6 | 1.16 |
| Lipid 5, DPG-PEG | 19.1 | −0.742 |
| Lipid 6, DPG-PEG | 13.5 | −1.9 |
| Lipid 7, DPG-PEG | 10.8 | −4.31 |
| Lipid 8, DPG-PEG | 20.6 | 0.051 |
| SM-102, DPG-PEG | 12.8 | −2.25 |
| ALC-0315, DPG-PEG | 4.25 | −2.95 |
| MC-3, DPG-PEG | 12.7 | −0.43 |
| KC-2, DPG-PEG | 22.7 | 1.41 |

TABLE 64
LNP charge (Zeta Potential) of Lipids 9, 10, 11, and 15. Charge
(Zeta Potential) (DLS, mV) in pH 5.5 MES and pH 7.4
HBS of LNPs with Lipids 9, 10, 11, and 15 and comparator
Lipids SM-102, ALC-0315, MC3, and KC3, respectively.

| Ionizable Lipid, | LNP charge (ZP, mV); | LNP charge (ZP, mV); |
|---|---|---|
| LNP no. | pH 5.5 | pH 7.4 |
| Lipid 9 | 17.2 | 0.0437 |
| Lipid 10 | 21 | −0.137 |
| Lipid 11 | 16.5 | −1.01 |
| Lipid 15 | 19.7 | −0.322 |
| SM-102 | 12.8 | −2.25 |
| ALC-0315 | 4.25 | −2.95 |
| DLn-MC3-DMA | 12.7 | −0.43 |
| DLn-KC2-DMA | 22.7 | 1.41 |

Introduction of a biodegradable ester linkage in the lipid head group (lipids 9, 10, 11 and 15) resulted in a stabilizing effect on LNP colloidal stability. N-acyl substituents derived from succinate linked branched aliphatic moieties (C8 of lipid 10 and C10 of lipids 9 and 15) resulted in colloidally stable particles. However, the α-hydroxyl substituted ester analog (in lipid 11) resulted in significant loss of LNP size stability and final (post-freeze/thaw) LNP diameters >150 nm. All lipids (except lipids 6 and 7) showed Polydispersity values <0.2 suggesting that the greater hydrophobicity of the head group in lipids 6 and 7 (both C11 N-acyl substituted) drives LNP aggregation upon freeze/thaw. As shown in Tables 65 and 66, all novel lipids tested resulted in high (>80%) RNA encapsulation and good process recoveries. Tables 63 and 64 summarize the LNP charge values, all ionizable lipids showed a relatively strong positive charge at acidic pH (5.5) and near neutral charge at physiological pH (as expected).

TABLE 65
RNA content of Lipids 1-8. Dye Accessible RNA and total RNA content of LNPs
with Lipids 1-8 and comparator Lipids SM-102, ALC-0315, MC3, and KC3, respectively.

| | Nominal | Measured | Dye-accessible | Total mRNA | Dye- |
|---|---|---|---|---|---|
| Ionizable Lipid, | mRNA Conc. | Total mRNA | mRNA | Recovery | accessible |
| LNP no. | (μg/mL) | (μg/mL) | (μg/mL) | (%) | mRNA (%) |
| Lipid 1, DPG-PEG | 100 | 75.5 | 8.9 | 75.5 | 11.8 |
| Lipid 2, DMG-PEG | 100 | 134.7 | 11.1 | 134.7 | 8.2 |
| Lipid 2, DPG-PEG | 100 | 136.9 | 9.4 | 136.9 | 6.9 |
| Lipid 3, DMG-PEG | 100 | 83.2 | 8.1 | 83.2 | 9.8 |
| Lipid 3, DPG-PEG | 100 | 93.5 | 10.3 | 93.5 | 11.0 |
| Lipid 4, DPG-PEG | 100 | 77.6 | 9 | 77.6 | 11.6 |
| Lipid 5, DPG-PEG | 100 | 73.4 | 11.8 | 73.4 | 16.1 |
| Lipid 6, DPG-PEG | 50 | 34.7 | 3.3 | 69.4 | 9.5 |
| Lipid 7, DPG-PEG | 50 | 38.9 | 4.3 | 77.8 | 11.1 |
| Lipid 8, DPG-PEG | 100 | 85.9 | 8.4 | 85.9 | 9.8 |
| SM-102, DPG-PEG | 50 | 28.7 | 4.3 | 57.4 | 15.0 |
| ALC-0315, DPG- | 50 | 38.1 | 4.8 | 76.2 | 12.6 |
| PEG | | | | | |
| MC-3, DPG-PEG | 50 | 36.8 | 1.8 | 73.6 | 4.9 |
| KC-2, DPG-PEG | 50 | 43 | 3.6 | 86 | 8.4 |

TABLE 66
RNA content of Lipids 9, 10, 11, and 15. Dye Accessible RNA and total RNA
content of LNPs with Lipids 9, 10, 11, and 15, respectively.

| Ionizable | Nominal | Measured | Dye-accessible | Total mRNA | Dye- |
|---|---|---|---|---|---|
| Lipid, LNP | mRNA Conc. | Total mRNA | mRNA | Recovery | accessible |
| no. | (μg/mL) | (μg/mL) | (μg/mL) | (%) | mRNA (%) |
| Lipid 9 | 100 | 84.4 | 5.4 | 84.4 | 6.4 |
| Lipid 10 | 100 | 80.2 | 6.6 | 80.2 | 8.2 |
| Lipid 11 | 100 | 85.6 | 13.6 | 85.6 | 15.9 |
| Lipid 15 | 100 | 74.9 | 5.1 | 74.9 | 6.8 |

In addition to the α-CD3 targeted LNPs described above, α-CD8 targeted LNPs based on lipids 3, 4, 5, 9, and 15 were produced by post-insertion (using a 4-hour incubation at 37° C.) of two α-CD8 targeting conjugates, namely, a TRX2 (an α-CD8 Fab conjugated to the PEG terminus of DSPE-PEG2k) and Nb8 (an α-CD8 VHH conjugated to the PEG terminus of DSPE-PEG3.4k). The resulting particle size characteristics (post-insertion and post-freeze/thaw) are summarized in Table 67. LNP diameters after the initial mixing step and subsequent buffer exchange into HBS measured ≤120 nm with Lipids 3, 9 and 15, while Lipid 5 LNPs exhibited >150 nm diameter. Notably, lipids 9 and 15 LNPs exhibited the smallest sizes (≤120 nm, PDI <0.2) after both αCD8 antibody conjugate insertions (TRX2 Fab conjugate and the Nb8 VHH conjugate) and after one freeze/thaw cycle, suggesting more robust LNP colloidal stability relative the LNPs based on lipids 3, 4 and 5. Thus, introduction of a biodegradable succinate-derived N-acyl substituent helps improve LNP colloidal stability under processing and freeze/thaw stress.

TABLE 67
LNP size and PDI of targeted LNPs. Size and PDI of LNPs with Lipids 3, 4, 5, 9,
and 15, pre- and post-insertion with aCD8 (TRX2 Fab and Nb8 VHH) targeting conjugates.

| | Z-Avg. | Z-Avg. | Z-Avg. Size | Z-Avg. Size | | | PDI (DLS); | PDI (DLS); |
|---|---|---|---|---|---|---|---|---|
| Ionizable | Size | Size | (nm); TRX2 | (nm); Nb8 | PDI | PDI | TRX2 Post- | Nb8; Post- |
| Lipid, LNP | (nm); | (nm); | post-insertion; | post-insertion; | (DLS); | (DLS); | insertion; | insertion; |
| no. | HBS | MBS | MBS | MBS | HBS | MBS | MBS | MBS |
| Lipid 3, | 101.5 | 110.8 | 117.4 | 131.0 | 0.08 | 0.09 | 0.116 | 0.166 |
| DPG-PEG, | | | | | | | | |
| TRX2 | | | | | | | | |
| Lipid 9, | 83.9 | 94.8 | 102.5 | 108.1 | 0.08 | 0.07 | 0.1255 | 0.143 |
| DPG-PEG, | | | | | | | | |
| TRX2 | | | | | | | | |
| Lipid 4, | 95.5 | 120.6 | 127.8 | 133.7 | 0.09 | 0.13 | 0.1325 | 0.114 |
| DPG-PEG, | | | | | | | | |
| TRX2 | | | | | | | | |
| Lipid 5, | 102.4 | 161.2 | 162.3 | 161.3 | 0.09 | 0.13 | 0.1335 | 0.095 |
| DPG-PEG, | | | | | | | | |
| TRX2 | | | | | | | | |
| Lipid | 91.55 | 97.65 | 106.0 | 115.1 | 0.07 | 0.06 | 0.1415 | 0.164 |
| 15; DPG- | | | | | | | | |
| PEG, TRX2 | | | | | | | | |

### Physiochemical Properties of Targeted LNPs Based on Lipids 10, 15, 16, 24A. 26 and ALC-0315

LNP size, polydispersity, charge, and RNA content values of Lipids 10, 15, 16, 24A, 26, and ALC-0315 LNPs are summarized herein (Tables 64, 68, 69, and 70, and FIGS. 60A-60D). As seen in FIG. 60A, all parent formulations based on Lipids 10, 15, 16, 24A and 26 resulted in LNP diameter <100 nm in pH 6.5 MES buffer. Lipid 10 and ALC-0315 LNPs exhibited a notable size increase upon freeze/thaw (with 10% sucrose in MES pH 6.5 buffer), while a minimal impact of one freeze/thaw cycle on LNP diameter was observed in Lipid 15, 16, 24A, and 26 LNPs. Similarly, relatively small changes in LNP diameter were observed upon targeting antibody insertion (in pH 6.5 MES using a 37° C. incubation for 4 hours) for Lipids 15, 16, 24A, and 26, while larger increase in LNP diameter was observed for Lipid 10 and ALC-0315 LNPs. The corresponding targeted LNPs also exhibited a similar trend in size changes upon one freeze/thaw cycle. All LNPs exhibited moderate to high encapsulation efficiency (<15% Dye accessible RNA), except for ALC-0315 LNPs, where higher dye accessible RNA was observed. Also, lipid 10, 15 and 16 LNPs exhibited strongly positive charge in acidic pH (5.5), while near neutral charge in physiological pH (7.4). In contrast, Lipids 24A, 26 an ALC-0315 LNPs exhibited a relatively weak positive charge in acidic pH (5.5) and a slight negative charge at physiological pH (7.4). The negative charge under physiological pH conditions (seen in LNPs based on lipids 14A, 26 and ALC-0315) suggests that the branched tail groups in this sub-set of lipids alters the interfacial presentation of the ionizable head group, thereby driving LNP apparent pKa towards lower values relative to the linoleic and oleic acid tail groups of lipids 10 and 15, respectively.

TABLE 68
LNP size and PDI of targeted LNPs. Size and PDI of LNPs with Lipids 10, 15, 16,
24A, 26, and ALC-0315, pre- and post-insertion with Nb8 VHH targeting conjugate.

| | | Z-Avg. Size | | | Polydispersity | |
|---|---|---|---|---|---|---|
| Ionizable | Z-Avg. | (nm); | Z-Avg. | Polydispersity | (DLS); | Polydispersity |
| lipid, LNP | Size (nm); | post-insertion; | Size (nm); | (DLS); | Post-insertion; | (DLS); |
| no. | MBS | MBS | Post F/T | MBS | MBS | Post F/T |
| Lipid 10, | 90 | 131 | 208 | 0.21 | 0.22 | 0.18 |
| DPG-PEG | | | | | | |
| Lipid 15, | 86 | 90 | 94 | 0.11 | 0.12 | 0.11 |
| DPG-PEG | | | | | | |
| Lipid 16, | 92 | 106 | 109 | 0.17 | 0.20 | 0.17 |
| DPG-PEG | | | | | | |
| Lipid 24A, | 82 | 91 | 94 | 0.10 | 0.12 | 0.16 |
| DPG-PEG | | | | | | |
| Lipid 26, | 83 | 87 | 93 | 0.07 | 0.07 | 0.09 |
| DPG-PEG | | | | | | |
| ALC-0315, | 93 | 111 | 136 | 0.12 | 0.13 | 0.14 |
| DPG-PEG | | | | | | |

TABLE 69
LNP charge (Zeta Potential) of Lipids 10, 15, 16, 24A,
and 26. Charge (Zeta Potential) (DLS, mV) in pH 5.5 MES
and pH 7.4 HBS of LNPs consisting of Ionizable Lipids
10, 15, 16, 24A, and 26 and comparator Lipid ALC-0315, respectively.

| | Charge (ZP, mV); | Charge (ZP, mV); |
|---|---|---|
| Ionizable lipid, LNP no. | pH 5.5 | pH 7.4 |
| Lipid 10, DPG-PEG | 25.8 | 0.8 |
| Lipid 15, DPG-PEG | 18.4 | −0.2 |
| Lipid 16, DPG-PEG | 24.5 | −0.6 |
| Lipid 24A, DPG-PEG | −0.7 | −5.9 |
| Lipid 26, DPG-PEG | 4.3 | −5.2 |
| ALC-0315, DPG-PEG | 4.4 | −10.5 |

TABLE 70
RNA content of Lipids 10, 15, 16, 24A, and 26. Dye Accessible
RNA and total RNA content of LNPs consisting of Ionizable
Lipids 10, 15, 16, 24A, and 26, respectively.

| | Nominal | Measured | Dye-accessible |
|---|---|---|---|
| Ionizable lipid, LNP | mRNA Conc. | Total mRNA | mRNA |
| no. | (μg/m L) | (μg/m L) | (%) |
| Lipid 10, DPG-PEG | 150 | 124.30 | 10.9 |
| Lipid 15, DPG-PEG | 150 | 117.60 | 10.3 |
| Lipid 16, DPG-PEG | 150 | 109.90 | 9.40 |
| Lipid 24A, DPG-PEG | 150 | 134.90 | 26.4 |
| Lipid 26, DPG-PEG | 150 | 124.40 | ≤6 (5.5) |
| ALC-0315, DPG-PEG | 150 | 88.50 | 14.70 |

### Effect of Lipid Head Group Hydrophobicity and Regio-Isomerism on In Vitro T Cell Transfection With Targeted LNPs Based on Lipids 1, 3, 5, and 8.

αCD3 (SP34 Fab) targeted GFP mRNA LNPs based on lipids 1, 3, 5, and 8 were dosed to primary human T cells at dose levels between 0.001 and 1 μg/mL in two independent experiments (each experiment included LNPs based on a comparator lipid DLn-KC2-DMA) and GFP expression was assessed by Flow Cytometry analysis at 24 hours. As seen in FIGS. 61A and 61B, and FIGS. 62A and 62B, the percent GFP+ cells and the Mean Fluorescence Intensity (MFI) of transfected T cells suggested that hydrophobicity of the N-acyl substituent impacts protein expression. Lipids 5 and 3 feature an 8 carbon, 9 carbon N-acyl group respectively, while lipids 1 and 8 feature a 10-carbon N-acyl substituent (α-ethyl octanoic acid in lipid 1 vs β-ethyl octanoic acid in lipid 8). The order of GFP expression levels observed was Lipid 5 LNP>Lipid 3 LNP>1 LNP. This indicates that the least hydrophobic N-acyl substituent (C8) resulted in the most efficient cytosolic delivery of the mRNA payload and a step wise drop in the efficiency result with C9, C10 substitution (of Lipid 3 and 1, respectively). This trend suggests that greater hydrophobicity of the N-acyl substituent either limits partitioning (and fusogenicity) into endosomal membranes or slows RNA release (dissociation of ionizable lipid from RNA) in the cytoplasm thereby limiting the availability of the RNA payload for ribosome engagement and protein translation. See FIGS. 61A-61E and FIGS. 62A-62C.

Additionally, the β-ethyl octanoic acid substitution (in lipid 8) significantly improves LNP activity compared to the α-ethyl octanoic acid substitution (in lipid 1) indicating an effect of positional isomerism. The improved performance of the β-ethyl octanoic acid substitution (in lipid 8) over α-ethyl octanoic acid substitution (in lipid 1) may be attributed to enhanced aliphatic hydrocarbon mobility (lower melting point) and enhanced fusogenicity towards anionic endosomal membranes.

With respect to the optimal lipid composition for RNA delivery to T cells, both LNP colloidal stability and LNP activity were considered. As described earlier, the 8-carbon N-acyl substitution (of lipid 5) was the most active lipid (resulting in the highest protein expression). However, Lipid 5 LNPs exhibited inferior size stability relative to the 9C (lipid 3) and 10C compositions (lipids 1 and 8) LNPs. Thus, considering both factors, the 9-carbon N-acyl composition (of lipid 3) or β-substituted 10C compositions were found optimal.

Effect of Head Group Hydrophobicity on In Vitro T Cell Transfection with Targeted LNPs Based On Lipids Derived from a Biodegradable Ester-Linked N-Acyl Substituent

Lipids 10 and 9 were designed to introduce a biodegradable ester linkage in the N-acyl substituent. Based on optimal hydrophobic content and effects of β-substitution described above, either an 8- or a 10-carbon (C8 or C10) aliphatic ester of 3-octanol (in lipid 10) or 4-decanol (in lipid 9) and succinic acid were chosen. The in vitro protein expression levels (in primary human T cells) resulting from αCD3 targeted, GFP mRNA LNPs based lipids 10 and 9 was compared to the expression levels from lipid 8 LNPs (a 10 carbon, β-substituted N-acyl composition without the succinate ester linkage) and a comparator lipid DLn-KC2-DMA. As seen in FIG. 63C, GFP expression in T cells ranked in the order lipid 10>lipid 9>lipid 8 indicating that succinate ester improves LNP performance. Furthermore, improved performance of lipid 10 LNPs over Lipid 9 LNPs indicates that the 3-octanol derived succinate ester results in improved expression over the 4-decanoate derived analog consistent with the trends in N-acyl substituent hydrophobicity observed between lipid 5 (8C) and lipid 3 (9C) LNPs as described above. See FIGS. 63A-63D.

Effect of Lipid Tail Unsaturation on In Vitro T Cell Transfection with Targeted LNPs Derived From Linoleic Acid and Oleic Acid Lipid Tail Groups

αCD3 (SP34 Fab) targeted GFP mRNA LNPs based on lipids 3, 4, 9, and 15 were dosed to primary human T cells at dose levels between 0.001 and 1 μg/mL (alongside comparator lipid, DLn-KC2-DMA LNPs) either after storage at 4° C. or after being stored frozen at −80° C. and thawed at room temperature prior to dosing. GFP expression was assessed by Flow Cytometry analysis at 24 hours. As seen in FIGS. 64A-64E, at all dose levels, performance improvements (both in terms of fraction of GFP+ T cells as reflected by % GFP+ cells and the copies of GFP protein produced on a per cell basis as reflected by GFP MFI values) were observed with lipid 4 (relative to lipid 3) and with lipid 15 (relative to lipid 9) indicating that the oleoyl tail groups (in Lipids 4 and 15) result in higher levels of protein expression over the corresponding linoleoyl tail groups (in Lipids 3 and 9) in the context of T cell targeted lipid nanoparticles. Ionizable lipids derived from linoleic acid tail (di-unsaturated) groups are reported to improve protein expression relative to lipids derived from either oleic acid (mono-unsaturated) or saturated fatty acid tails. Linoleic acid-derived lipid tails are expected to lower lipid melting point (Tₘ) and favor phase transition of the lipid bilayer towards an inverted hexagonal H_{II} phase via ionizable lipid fusion with anionic lipids of the endosomal membranes. Thus, promoting endosomal membrane disruption and improved cytosolic delivery of the RNA payload. However, the phenomenon is not universal as illustrated here by the improved performance of lipid 5 (over lipid 4) and of lipid 15 (over lipid 9) LNPs. Furthermore, in contrast to Lipid 4 where poorer LNP size distribution properties were observed (relative to Lipid 3), Lipid 15 LNPs exhibited size distribution properties similar to those observed with Lipid 9 LNPs. Thus, while the di-oleoyl tail group does not affect size stability in the context of the decan-4-ol succinate N-acyl substituent (of lipid 15), it adversely impacted size stability in lipid 4 in the context of the 3-carboxyoctane derived N-acyl substituent. This suggests that the LNP size distribution (and in-process size stability) is dependent both on the chemistry of lipid tail group as well as of the N-acyl substituent. The performance levels and relative order of LNP efficiency were well preserved in all formulations tested after 1 freeze/thaw cycle (Post −80° C. storage) as illustrated by the comparison of % GFP+ cells and GFP MFI values before and after −80° C. storage (FIG. 64A versus FIG. 64B, and FIG. 64C versus FIG. 64D. Lipids 3, 4, 9, and 15 LNPs were well tolerated by primary human T cells (FIG. 64E).

Effect of Lipid Tail Unsaturation on In Vitro T Cell Transfection with LNPs Based on Lipids 3, 4, 9, and 15, and Evidence of Receptor-Mediated Uptake into Primary Human T Cells

Parent LNPs based on lipids 3, 4, 9, and 15 as well the corresponding targeted LNPs resulting from post insertion of αCD8 V_{HH} Nb8 were dosed to primary human T cells at dose levels between 0.001 and 1 μg/mL, and GFP expression was assessed by Flow Cytometry analysis at 24 hours. As seen in FIG. 65A and FIG. 65B, at all dose levels, Lipid 4 and Lipid 15 outperformed Lipid 3 and Lipid 9 LNPs, consistent with the results described above with the αCD3 SP34 Fab-targeted LNPs. Additionally, parent LNPs (dosed at 1 μg/mL only) resulted in no significant expression, thus confirming the role of receptor-mediated uptake into T cells. Lipids 3, 4, 9, and 15 used in αCD8 V_{HH} Nb8-targeted LNPs were well-tolerated by primary human T cells (FIG. 65E) with cell viability trending measurably lower in Lipid 9 and Lipid 15 formulations possibly due to the higher levels of GFP protein expression (as indicated by the higher GFP MFI values seen FIG. 65B). See FIGS. 65A-65E.

Effect of Tail Group Composition (Unsaturation and Branching) on In Vitro GFP Protein Expression in Primary Human T Cells Transfected with αCD8 (15C01) Targeted LNPs Based On Lipids 10, 15, 16, 24A, 26, and ALC-0315

Lipids 10, 15, 16, 24A, 26, and ALC-0315, αCD8 (Nb8 V_{HH}) targeted LNPs were well-tolerated by primary human T cells (FIG. 66E). As seen in FIGS. 66C and 66D, αCD8 Nb8-targeted LNPs based on lipids 10, 15, 16, 24A, 26, and ALC-0315 showed similar high % DiI+ (dye) T cells and Dil MFI reflective of equally efficient LNP association with the CD8+ T cell population. However, notable differences in GFP protein expression levels (as reflected by % GFP+ cells and the copies of GFP protein produced on a per cell basis as reflected by GFP MFI values) were observed (see FIGS. 66A and 66B). Lipid 15 LNPs outperformed the other Lipids within the group, with Lipids 26 and ALC-0315 also enabling relatively high levels of protein expression. It is noteworthy that Lipid 15 and Lipid 16 LNP Zeta Potential values at pH 5.5 and pH 7.4 suggest a large shift in charge and consequently a strong ability for fusion with endosomal membranes (and endosome disruption/endosomal escape) upon acidification of the endosomal compartment. However, Lipid 26 and ALC-0315 LNP Zeta Potential values suggest a relatively small charge shift under endosomal acidification. Hence, despite potentially a lower ability for charge-driven endosomal membrane destabilization, Lipid 26 and ALC-0315 LNPs enabled high levels of protein expression reflective of potent cytosolic delivery of the GFP RNA payload. This suggests that the more pronounced “cone” structure of the branched tail group and greater lipid cross-sectional area (in lipids 26 and ALC-0315) favors lipid bilayer transition to the Hexagonal Hu phase upon ionizable lipid fusion with negatively charged endosomal membrane lipids, thus, enabling efficient endosomal membrane disruption, cytosolic availability of the RNA payload, and higher levels of protein expression.

### Jurkat T Cell Activation Assay

The human T cell line, Jurkat, Clone E6-1, was purchased from ATCC and was maintained according to the manufacturer's recommendations. Jurkat T cells were transfected with CAR-encoding mRNA by electroporation. Briefly, 10⁵ Jurkat T cells were resuspended in 20 μL SE 4D-Nucleofector™ X Solution (Lonza) and mixed with 0.6 μg mRNA. The T cells were then electroporated in 20 μL Nucleocuvette strips using a 4D-Nucleofector X Unit (Lonza). Following a 2-hour rest period post-electroporation, Jurkat T cells were co-cultured with Raji cells at effector-to-target cell ratios of 4:1. 24 hours after co-culture, T cells were stained with CD3-APC (BD Biosciences) and CD69-BV421 (BD Biosciences). Dead cells were excluded from the analysis by using eFluor780 fixable viability dye (Thermo Fisher). CAR-mediated activation was defined as the CD69 MFI level in live CD3⁺ cells (signal) divided by the CD69 MFI level in a group without Raji cells (background).

### Cytotoxicity Against Cell Lines and Quantification of Surface Markers

Cells from various cell lines (e.g., CD19+ cell lines: Nalm6 and Raji; CD19-cell lines: K562; see Table 71) were stained with Cell Trace Violet proliferation dye (CTV) (Thermo Fisher) following the manufacturer's protocol. Briefly, target cells were incubated with CTV for 20 minutes at room temperature prior to washing and co-culturing. CD8⁺ T cells were isolated and transfected as described above and washed after 2 hours of incubation to remove unbound LNPs. Transfected CD8⁺ T cells were then co-cultured with CTV-stained target cells at varying effector-to-target cell ratios in T cell media (RPMI-1640, 10% FBS, and 50 ng/ml recombinant human IL-2) in a 96-well flat-bottom plate for 48 hours. After 48 hours of co-culture, cells were stained with eFluor780 fixable viability dye (Thermo Fisher) to assess the level of cytotoxicity. Cells were washed twice in FACS buffer and acquired by Flow Cytometry on a Penteon Novocyte (Agilent) and further analyzed in FlowJo, where dead target cells were defined as CTV⁺ and Live/Dead eFluor780⁺ cells.

A Quantibrite PE kit (BD Biosciences) was used to quantify the number of CD19 and CD22 molecules per cell, respectively, on each cell line following the manufacturer's instructions.

TABLE 71
Receptor quantification and EC50 values in various target
cell lines (CD19 and CD22 expression density,
and FMC63 cytotoxicity in selected target cell lines)

| | FMC63 | CD19 | CD22 | |
|---|---|---|---|---|
| Cell line | EC50 | density | density | Media |
| Nalm6 | 0.89 | 35740 | 2741 | RPMI 10% FBS |
| Raji | 6.78 | 57232 | 10322 | RPMI 10% FBS |
| K562 | — | 33 | 48 | RPMI 10% FBS |

### Amnis® ImageStream Assay

### Sample Acquisition

Samples were acquired on a Cytek® Amnis® ImageStream®X Mk II (Cytek Biosciences, Fremont, CA, USA) equipped with 2 cameras and 4 lasers. Data was acquired using Cytek® Amnis® INSPIRE™ acquisition software. The system was calibrated with ASSIST (Automated Suite of System-wide ImageStreamX Tests) using the INSPIRE software.

To maximize signal detection and avoid saturation of the cameras, the following settings were utilized: 120 mW 405 nm violet laser, 150 mW 488 nm blue laser, 150 mW 642 nm red laser and 2 mW 785 nm NIR laser (for side scatter). The images were acquired using slow speed (highest sensitivity) with a magnification of 40×(0.5 um2/pixel resolution). Bright-field imagery was collected using an LED array with wavelengths of 430 nm to 470 nm in channel 1 (brightfield 1) and 575 nm to 595 nm in channel 9 (brightfield 2). Side scatter was collected in channel 6 using the dedicated 785 nm laser.

Controls for compensation were acquired with brightfield and side scatter illumination sources off and with all imaging channels enabled.

The staining panel included Channel 1, Em Band 430-470 for Brightfield-Morphology; Channel 2, Ex 488, Em Band 470-560 for FITC-CD8; Channel 3, Ex 488/561, Em Band 560-595 for PE-CAR; Channel 6, Ex 488/561, Em Band 745-800 for SSC-Granularity; Channel 7, Ex 405, Em Band 430-505 for CellTrace Violet-Nalm6 cells; Channel 9, Ex 405, Em Band 575-595 for Brightfield-Morphology; Channel 11, Ex 642/405, Em Band 660-720 for BV711-CD3; Channel 12, Ex 642/405, Em Band 720-800 for eFluor780-Viability.

Approximately 50,000 objects were recorded per sample, based on objects displaying a larger area than the ASSIST SpeedBeads in the Area vs Aspect ratio plot of the BF channel (Ch01). For each sample, data was saved in raw image file (.rif) format then analyzed in Cytek® Amnis® IDEAS® v6.4+Machine Learning (ML) software.

### Interacting Cell Analysis Using IDEAS® v6.4+ML

IDEAS® v6.4 is a cellular analysis software for the Cytek® Amnis® ImageStreamRX Mk II imaging flow cytometer. The software allows for quantification of cellular activity by performing statistical analyses on thousands of events and, at the same time, permits visual confirmation of any individual event.

The single-color compensation controls acquired during acquisition were used to correct for fluorescence emission spillover into off-target detection channels and generate a compensation matrix. The matrix was applied to the fully stained samples and the compensated data was analyzed.

Doublet cells were identified by generating a gate on “In Focus” objects (Gradient RMS Brightfield >50) and “doublet” objects (Area Brightfield >220 μm² and Aspect Ratio Brightfield <0.77). To confirm the presence of CellTrace Violet (B Cells) and CD3 doublet events, an intensity plot was created, and double positive objects were selected. Dead cells were removed from the analysis with a negative selection gate on the intensity of Live/Dead Far Red. The “In Focus”, “Doublet”, “CellTrace Violet+, CD3+”, and “Live” objects were further observed for the total intensity of CAR versus the maximum pixel intensity of CAR. CAR+ events were identified as total intensity >1600 RFUs and max pixel intensity >97 RFUs. The final step in the analysis was to determine if the B and T cells were interacting.

The machine learning (ML) module for the Cytek® Amnis® IDEAS® software creates an experiment-specific feature that distinguishes populations based on user input. The module is built upon a modified version of the linear discriminant analysis (LDA) method and is integrated into Cytek® Amnis® IDEAS® v6.4. The module leverages available feature and masking algorithms, normalizes all features to a common base (to offset the wide variation in feature value ranges), uses Fisher's Discriminant Ratio for feature selection then uses a weighted linear combination of top-selected features to generate a linear classifier based upon input populations.

Twenty-five positive B cell-T cell interactions and 25 negative B cell-T cell interactions were individually tagged via the software tagging tool. The tagged populations were analyzed using the machine learning module while specifying “In Focus”, “Doublet”, “CellTrace Violet+, CD3+”, “Live”, “CAR+” objects as the base population. Using the B cell—T cell positive interaction classifier, objects yielding a positive value (greater than 0) were gated as interacting and those with a negative value (less than 0) were gated as not interacting. Features used in the classifier consisted of aspect ratio Brightfield (BF), circularity (BF), major axis (BF), and symmetry BF.

To validate that the classifier was accurate, a template of the training sample analysis was applied to untrained files. All samples were batch-analyzed and a statistics report was generated.

SEQUENCE TABLE

| SEQ | | |
|---|---|---|
| ID NO | Description | Sequence |
| 1 | A044300805 | EVQLVESGGGVVQPGGSLRLSCAASGFTFEDYAIGWFRQAPGKEREEVSCIRTYDEQTYYAD |
| | | SVKGRFTISRDNAKNTVYLQMNSLRPEDTALYYCAAGSYYACAKYSRPDPSEGHVDLDYW |
| | | GQGTLVTVSS |
| 2 | A044300805_v1 | EVQLVESGGGVVQPGGSLRLSCAASGFTFEDYAIGWFRQAPGKEREEVSCIRTYDEQTYYAD |
| | | SVKGRFTISRDNAKNTVYLQMNSLRPEDTALYYCAAGSYYACAEYSRPDPSEGHVDLDYWG |
| | | QGTLVTVSS |
| 3 | A044300805_v2 | EVQLVESGGGVVQPGGSLRLSCAASGFTFEDYAIGWFRQAPGKEREEVSCIRTYDEQTYYAD |
| | | SVKGRFTISRDNAKNTVYLQMNSLRPEDTALYYCAAGSYYACAYYSRPDPSEGHVDLDYW |
| | | GQGTLVTVSS |
| 4 | A044300805_v3 | EVQLVESGGGVVQPGGSLRLSCAASGFTFEDYAIGWFRQAPGKEREEVSCIRTYDEQTYYAD |
| | | SVKGRFTISRDNAKNTVDLQMNSLRPEDTALYYCAAGSYYACAKYSRPDPSEGHVDLDYW |
| | | GQGTLVTVSS |
| 5 | A044300805_v4 | EVQLVESGGGVVQPGGSLRLSCAASGFTFEDYAIGWFRQAPGKEREEVSCIRTYDEQTYYAD |
| | | SVKGRFTISRDNAKNTVDLQMNSLRPEDTALYYCAAGSYYACAEYSRPDPSEGHVDLDYWG |
| | | QGTLVTVSS |
| 6 | A044300805_v5 | EVQLVESGGGVVQPGGSLRLSCAASGFTFEDYAIGWFRQAPGKEREEVSCIRTYDEQTYYAD |
| | | SVKGRFTISRDNAKNTVDLQMNSLRPEDTALYYCAAGSYYACAYYSRPDPSEGHVDLDYW |
| | | GQGTLVTVSS |
| 7 | A044300805_v6 | EVQLVESGGGVVQPGGSLRLSCAASGFTFEDYAIGWFRQAPGKEREEVSCIRTYDEQTYYAD |
| | | SVKGRFTISRDNAKNTVSLQMNSLRPEDTALYYCAAGSYYACAKYSRPDPSEGHVDLDYWG |
| | | QGTLVTVSS |
| 8 | A044300805_v7 | EVQLVESGGGVVQPGGSLRLSCAASGFTFEDYAIGWFRQAPGKEREEVSCIRTYDEQTYYAD |
| | | SVKGRFTISRDNAKNTVSLQMNSLRPEDTALYYCAAGSYYACAEYSRPDPSEGHVDLDYWG |
| | | QGTLVTVSS |
| 9 | A044300805_v8 | EVQLVESGGGVVQPGGSLRLSCAASGFTFEDYAIGWFRQAPGKEREEVSCIRTYDEQTYYAD |
| | | SVKGRFTISRDNAKNTVSLQMNSLRPEDTALYYCAAGSYYACAYYSRPDPSEGHVDLDYWG |
| | | QGTLVTVSS |
| 10 | A044300694 | EVQLVESGGGVVQPGGSLRLSCAASGSTFSDYGVGWFRQAPGKGREFVADIDWEGEHTSYA |
| | | DSVKGRFTISRDNAKNTAYLQMNSLRPEDTALYYCAADALPYTVRKYRYWGQGTLVTVSS |
| 11 | A044300694_v1 | EVQLVESGGGVVQPGGSLRLSCAASGSTFSDYGVGWFRQAPGKGREFVADIDWEGEHTSYA |
| | | DSVKGRFTISRDNAKNTAYLQMNSLRPEDTALYYCAEDALPYTVRKYRYWGQGTLVTVSS |
| 12 | A044300694_v2 | EVQLVESGGGVVQPGGSLRLSCAASGSTFSDYGVGWFRQAPGKGREFVADIDWEGEHTSYA |
| | | DSVKGRFTISRDNAKNTAYLQMNSLRPEDTALYYCAYDALPYTVRKYRYWGQGTLVTVSS |
| 13 | A044300694_v3 | EVQLVESGGGVVQPGGSLRLSCAASGSTFSDYGVGWFRQAPGKGREFVADIDWEGEHTSYA |
| | | DSVKGRFTISRDNAKNTAYLQMNSLRPEDTALYYCAADALPYTLRKYRYWGQGTLVTVSS |
| 14 | A044300694_v4 | EVQLVESGGGVVQPGGSLRLSCAASGSTFSDYGVGWFRQAPGKGREFVADIDWEGEHTSYA |
| | | DSVKGRFTISRDNAKNTAYLQMNSLRPEDTALYYCAEDALPYTLRKYRYWGQGTLVTVSS |
| 15 | A044300694_v5 | EVQLVESGGGVVQPGGSLRLSCAASGSTFSDYGVGWFRQAPGKGREFVADIDWEGEHTSYA |
| | | DSVKGRFTISRDNAKNTAYLQMNSLRPEDTALYYCAYDALPYTLRKYRYWGQGTLVTVSS |
| 16 | A044300694_v6 | EVQLVESGGGVVQPGGSLRLSCAASGSTFSDYGVGWFRQAPGKGREFVADIDWEGEHTSYA |
| | | DSVKGRFTISRDNAKNTADLQMNSLRPEDTALYYCAADALPYTVRKYRYWGQGTLVTVSS |
| 17 | A044300694_v7 | EVQLVESGGGVVQPGGSLRLSCAASGSTFSDYGVGWFRQAPGKGREFVADIDWEGEHTSYA |
| | | DSVKGRFTISRDNAKNTADLQMNSLRPEDTALYYCAADALPYTLRKYRYWGQGTLVTVSS |
| 18 | A044300694_v8 | EVQLVESGGGVVQPGGSLRLSCAASGSTFSDYGVGWFRQAPGKGREFVADIDWEGEHTSYA |
| | | DSVKGRFTISRDNAKNTADLQMNSLRPEDTALYYCAEDALPYTLRKYRYWGQGTLVTVSS |
| 19 | A044300694_v9 | EVQLVESGGGVVQPGGSLRLSCAASGSTFSDYGVGWFRQAPGKGREFVADIDWEGEHTSYA |
| | | DSVKGRFTISRDNAKNTADLQMNSLRPEDTALYYCAYDALPYTLRKYRYWGQGTLVTVSS |
| 20 | A044300694_v10 | EVQLVESGGGVVQPGGSLRLSCAASGSTFSDYGVGWFRQAPGKGREFVADIDWEGEHTSYA |
| | | DSVKGRFTISRDNAKNTADLQMNSLRPEDTALYYCAEDALPYTVRKYRYWGQGTLVTVSS |
| 21 | A044300694_v11 | EVQLVESGGGVVQPGGSLRLSCAASGSTFSDYGVGWFRQAPGKGREFVADIDWEGEHTSYA |
| | | DSVKGRFTISRDNAKNTADLQMNSLRPEDTALYYCAYDALPYTVRKYRYWGQGTLVTVSS |
| 22 | A044300694_v12 | EVQLVESGGGVVQPGGSLRLSCAASGSTFSDYGVGWFRQAPGKGREFVADIDWEGEHTSYA |
| | | DSVKGRFTISRDNAKNTASLQMNSLRPEDTALYYCAADALPYTLRKYRYWGQGTLVTVSS |
| 23 | A044300694_v13 | EVQLVESGGGVVQPGGSLRLSCAASGSTFSDYGVGWFRQAPGKGREFVADIDWEGEHTSYA |
| | | DSVKGRFTISRDNAKNTASLQMNSLRPEDTALYYCAEDALPYTLRKYRYWGQGTLVTVSS |
| 24 | A044300694_v14 | EVQLVESGGGVVQPGGSLRLSCAASGSTFSDYGVGWFRQAPGKGREFVADIDWEGEHTSYA |
| | | DSVKGRFTISRDNAKNTASLQMNSLRPEDTALYYCAYDALPYTLRKYRYWGQGTLVTVSS |
| 25 | A044300694_v15 | EVQLVESGGGVVQPGGSLRLSCAASGSTFSDYGVGWFRQAPGKGREFVADIDWEGEHTSYA |
| | | DSVKGRFTISRDNAKNTASLQMNSLRPEDTALYYCAADALPYTVRKYRYWGQGTLVTVSS |
| 26 | A044300694_v16 | EVQLVESGGGVVQPGGSLRLSCAASGSTFSDYGVGWFRQAPGKGREFVADIDWEGEHTSYA |
| | | DSVKGRFTISRDNAKNTASLQMNSLRPEDTALYYCAEDALPYTVRKYRYWGQGTLVTVSS |
| 27 | A044300694_v17 | EVQLVESGGGVVQPGGSLRLSCAASGSTFSDYGVGWFRQAPGKGREFVADIDWEGEHTSYA |
| | | DSVKGRFTISRDNAKNTASLQMNSLRPEDTALYYCAYDALPYTVRKYRYWGQGTLVTVSS |
| 28 | 10347015C01_GGC | EVQLVESGGGLVQAGGSLRLSCAASGFTFDDYAIGWFRQAPGKEREEVSCIRTYDGNTYYID |
| | | SVKGRFTISSDNAKNTVYLQMNSLKPEDTAAYYCAAGSYYACAKYSRPDPSENHVDMDYW |
| | | GKGTLVTVSSGCC |
| 29 | A044300805_GGC | EVQLVESGGGVVQPGGSLRLSCAASGFTFEDYAIGWFRQAPGKEREEVSCIRTYDEQTYYAD |
| | | SVKGRFTISRDNAKNTVYLQMNSLRPEDTALYYCAAGSYYACAKYSRPDPSEGHVDLDYW |
| | | GQGTLVTVSSGGC |
| 30 | A044300805_v1_GGC | EVQLVESGGGVVQPGGSLRLSCAASGFTFEDYAIGWFRQAPGKEREEVSCIRTYDEQTYYAD |
| | | SVKGRFTISRDNAKNTVYLQMNSLRPEDTALYYCAAGSYYACAEYSRPDPSEGHVDLDYWG |
| | | QGTLVTVSSGGC |
| 31 | A044300805_v2_GGC | EVQLVESGGGVVQPGGSLRLSCAASGFTFEDYAIGWFRQAPGKEREEVSCIRTYDEQTYYAD |
| | | SVKGRFTISRDNAKNTVYLQMNSLRPEDTALYYCAAGSYYACAYYSRPDPSEGHVDLDYW |
| | | GQGTLVTVSSGGC |
| 32 | A044300805_v3_GGC | EVQLVESGGGVVQPGGSLRLSCAASGFTFEDYAIGWFRQAPGKEREEVSCIRTYDEQTYYAD |
| | | SVKGRFTISRDNAKNTVDLQMNSLRPEDTALYYCAAGSYYACAKYSRPDPSEGHVDLDYW |
| | | GQGTLVTVSSGGC |
| 33 | A044300805_v4_GGC | EVQLVESGGGVVQPGGSLRLSCAASGFTFEDYAIGWFRQAPGKEREEVSCIRTYDEQTYYAD |
| | | SVKGRFTISRDNAKNTVDLQMNSLRPEDTALYYCAAGSYYACAEYSRPDPSEGHVDLDYWG |
| | | QGTLVTVSSGGC |
| 34 | A044300805_v5_GGC | EVQLVESGGGVVQPGGSLRLSCAASGFTFEDYAIGWFRQAPGKEREEVSCIRTYDEQTYYAD |
| | | SVKGRFTISRDNAKNTVDLQMNSLRPEDTALYYCAAGSYYACAYYSRPDPSEGHVDLDYW |
| | | GQGTLVTVSSGGC |
| 35 | A044300805_v6_GGC | EVQLVESGGGVVQPGGSLRLSCAASGFTFEDYAIGWFRQAPGKEREEVSCIRTYDEQTYYAD |
| | | SVKGRFTISRDNAKNTVSLQMNSLRPEDTALYYCAAGSYYACAKYSRPDPSEGHVDLDYWG |
| | | QGTLVTVSSGGC |
| 36 | A044300805_v7_GGC | EVQLVESGGGVVQPGGSLRLSCAASGFTFEDYAIGWFRQAPGKEREEVSCIRTYDEQTYYAD |
| | | SVKGRFTISRDNAKNTVSLQMNSLRPEDTALYYCAAGSYYACAEYSRPDPSEGHVDLDYWG |
| | | QGTLVTVSSGGC |
| 37 | VHH6 | DVQLQESGGGLVQAGGSLRLSCAVSGYPYSSYCMGWFRQAPGKEREGVAAIDSDGRTRYA |
| | | DSVKGRFTISQDNAKNTLYLQMNRMKPEDTAMYYCAARFGPMGCVDLSTLSFGHWGQGTQ |
| | | VTVSITTSFVPVFLPAKPTTTPAPRPPTPAPTIASQPLSLRPEASRPAAGGAVHTRGLDFAGGCG |
| | | GHHHHHH |
| 38 | TRX2 bDS | QVQLVESGGGVVQPGRSLRLSCAASGFTFSDFGMNWVRQAPGKGLEWVALIYYDGSNKFY |
| | (HC) | ADSVKGRFTISRDNSKNTLYLQMNSLRAEDTAVYYCAKPHYDGYYHFFDSWGQGTLVTVSS |
| | | ASTKGPSVFPLAPSSKSTSGGTAALGCLVKDYFPEPVTVSWNSGALTSGVHTCPAVLQSSGLY |
| | | SLSSVVTVPSSSLGTQTYICNVNHKPSNTKVDKKVEPKSSDKTHTC |
| 39 | TRX2 bDS (LC) | QTVVTQEPSLTVSPGGTVTLTCGSSTGAVTSGNYPNWVQQKPGQAPRGLIGGTKFLAPGTPA |
| | | RFSGSLLGGKAALTLSGVQPEDEAEYYCVLWYSNRWVFGGGTKLTVLSQPKAAPSVTLFPPS |
| | | SEELQANKATLVCLVSDFYPGAVTVAWKADGSPVKVGVETTKPSKQSNNKYAACSYLSLTP |
| | | EQWKSHRSYSCRVTHEGSTVEKTVAPAESS |
| 40 | hSP34 bDS (HC) | EVQLVESGGGLVQPGGSLKLSCAASGFTFNKYAMNWVRQAPGKGLEWVARIRSKYNNYAT |
| | | YYADSVKDRFTISRDDSKNTAYLQMNNLKTEDTAVYYCVRHGNFGNSYISYWAYWGQGTL |
| | | VTVSSASTKGPSVFPLAPSSKSTSGGTAALGCLVKDYFPEPVTVSWNSGALTSGVHTCPAVLQ |
| | | SSGLYSLSSVVTVPSSSLGTQTYICNVNHKPSNTKVDKKVEPKSSDKTHTC |
| 41 | hSP34 bDS (LC) | QTVVTQEPSLTVSPGGTVTLTCGSSTGAVTSGNYPNWVQQKPGQAPRGLIGGTKFLAPGTPA |
| | | RFSGSLLGGKAALTLSGVQPEDEAEYYCVLWYSNRWVFGGGTKLTVLSQPKAAPSVTLFPPS |
| | | SEELQANKATLVCLVSDFYPGAVTVAWKADGSPVKVGVETTKPSKQSNNKYAACSYLSLTP |
| | | EQWKSHRSYSCRVTHEGSTVEKTVAPAESS |
| 42 | mutOKT8 NoDS (HC) | QVQLVQSGAEDKKPGASVKVSCKASGFNIKDTYIHWVRQAPGQGLEWMGRIDPANDNTLY |
| | | ASKFQGRVTITADTSSNTAYMELSSLRSEDTAVYYCGRGAGYYVFDHWGQGTTVTVSSAST |
| | | KGPSVFPLAPSSKSTSGGTAALGCLVKDYFPEPVTVSWNSGALTSGVHTFPÅVLQSSGLYSLS |
| | | SVVTVPSSSLGTQTYICNVNHKPSNTKVDKKVEPKSSDKTHTC |
| 43 | mutOKT8 NoDS (LC) | DIVMTQSPSSLSASVGDRVTITCRTSRSISQALAWYQEKPGKAPKLLIYSGSTLQSGVPSRFSG |
| | | SGSGTDFTLTISSLQPEDFATYYCQQHNENPLTFGQGTKVEIKRTVAAPSVFIFPPSDEQLKSG |
| | | TASVVCLLNNFYPREAKVQWKVDNALQSGNSQESVTEQDSKDSTYSLSSTLTLSKADYEKH |
| | | KVYACEVTHQGLSSPVTKSFNRGES |
| 44 | A044300805_v8_GGC | EVQLVESGGGVVQPGGSLRLSCAASGFTFEDYAIGWFRQAPGKEREEVSCIRTYDEQTYYAD |
| | | SVKGRFTISRDNAKNTVSLQMNSLRPEDTALYYCAAGSYYACAYYSRPDPSEGHVDLDYWG |
| | | QGTLVTVSSGGC |
| 45 | PolyA | AAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAA |
| 46 | Anti-CD19 CAR #1 nucleic | ATGGCTCTGCCCGTGACCGCACTGCTGCTGCCCCTGGCCCTGCTGCTGCACGCCGCCAGG |
| | acid sequence | CCCGAGGTGCAGCTGGTGGAGTCCGGCGGCGGCGTGGTGCAGCCAGGCGGCTCTCTGAG |
| | | GCTGAGCTGCGCCGCCAGCGGCCTATTTCTAAGCGACTACACGCTAGCCTGGTTCAGGCA |
| | | GGCCCCCGGCAAGGAGCGCGAGTTTGTGATCGTTAGCACGTGGAACCGCTATACCTACT |
| | | ACGCTGAAGGAGTGAAGGGCAGGTTCACAATTTCTCGGGACAACGCCAAGAAAACCGTG |
| | | TATCTGCAGATGAACTCCCTGCGCCCCGAGGACACAGCCCTGTACTACTGCGCCGCAAAA |
| | | TTTCGCATCTCATACAACGTTAGGGACGACAGCTTCTACGACCTGTGGGGACAGGGCACC |
| | | CTGGTGACCGTGTCCTCCGCCGCCGCTATCGAGGTGATGTACCCTCCCCCTTACCTGGAC |
| | | AACGAGAAATCAAACGGCACCATCATCCACGTGAAGGGAAAGCACCTGTGTCCTAGCCC |
| | | ACTGTTCCCAGGCCCCAGCAAGCCCTTTTGGGTGCTGGTTGTGGTGGGCGGCGTGCTGGC |
| | | CTGCTACAGCCTGCTGGTCACCGTGGCCTTCATCATCTTCTGGGTTCGGTCCAAGCGCTCA |
| | | CGCCTGCTACACTCAGACTACATGAACATGACGCCCCGCAGACCTGGACCTACCAGGAA |
| | | ACACTATCAACCATATGCCCCCCCAAGGGACTTTGCAGCATACAGGAGCCGCGTGAAGT |
| | | TCTCCAGGAGCGCCGATGCCCCCGCCTACCAGCAGGGCCAGAACCAGCTGTACAACGAG |
| | | CTGAACCTGGGCCGGCGGGAGGAATACGACGTGCTGGACAAGCGGAGAGGCCGGGACC |
| | | CTGAGATGGGCGGAAAGCCTAGACGCAAGAACCCCCAGGAGGGCCTGTACAACGAGCT |
| | | GCAGAAAGATAAAATGGCCGAGGCCTATAGCGAGATCGGCATGAAGGGCGAGAGAAGA |
| | | CGCGGCAAGGGGCACGATGGCCTGTACCAGGGGCTGAGCACCGCCACCAAAGACACCTA |
| | | CGATGCCCTGCACATGCAGGCCCTGCCCCCGCGGTAATAATAA |
| 47 | Anti-CD19 CAR #2 nucleic | ATGGCTCTGCCCGTGACCGCACTGCTGCTGCCCCTGGCCCTGCTGCTGCACGCCGCCAGG |
| | acid sequence | CCCGAGGTGCAGCTGGTGGAGTCCGGCGGCGGCGTGGTGCAGCCAGGCGGCTCTCTGAG |
| | | GCTGAGCTGCGCCGCCAGCGGCCTATTTCTAAGCGACTACACGCTAGCCTGGTTCAGGCA |
| | | GGCCCCCGGCAAGGAGCGCGAGTTTGTGATCGTTAGCACGTGGAACCGCTATACCTACT |
| | | ACGCTGAAGGAGTGAAGGGCAGGTTCACAATTTCTCGGGACAACGCCAAGAAAACCGTG |
| | | TATCTGCAGATGAACTCCCTGCGCCCCGAGGACACAGCCCTGTACTACTGCGCCGCAAAA |
| | | TTTCGCATCTCATACAACGTTAGGGACGACAGCTTCTACGACCTGTGGGGACAGGGCACC |
| | | CTGGTGACCGTGTCCTCCGCCGCCGCTATCGAGGTGATGTACCCTCCCCCTTACCTGGAC |
| | | AACGAGAAATCAAACGGCACCATCATCCACGTGAAGGGAAAGCACCTGTGTCCTAGCCC |
| | | ACTGTTCCCAGGCCCCAGCAAGCCCTTTTGGGTGCTGGTTGTGGTGGGCGGCGTGCTGGC |
| | | CTGCTACAGCCTGCTGGTCACCGTGGCCTTCATCATCTTCTGGAAGCGGGGACGCAAGAA |
| | | GCTGCTGTATATCTTTAAGCAACCATTCATGCGGCCCGTTCAAACGACTCAAGAAGAAGA |
| | | CGGGTGTAGCTGCCGGTTTCCCGAAGAAGAAGAGGGCGGCTGTGAACTACGCGTGAAGT |
| | | TCTCCAGGAGCGCCGATGCCCCCGCCTACCAGCAGGGCCAGAACCAGCTGTACAACGAG |
| | | CTGAACCTGGGCCGGCGGGAGGAATACGACGTGCTGGACAAGCGGAGAGGCCGGGACC |
| | | CTGAGATGGGCGGAAAGCCTAGACGCAAGAACCCCCAGGAGGGCCTGTACAACGAGCT |
| | | GCAGAAAGATAAAATGGCCGAGGCCTATAGCGAGATCGGCATGAAGGGCGAGAGAAGA |
| | | CGCGGCAAGGGGCACGATGGCCTGTACCAGGGGCTGAGCACCGCCACCAAAGACACCTA |
| | | CGATGCCCTGCACATGCAGGCCCTGCCCCCGCGGTAATAATAA |
| 48 | Anti-CD19 CAR #3 nucleic | ATGGCTCTGCCCGTGACCGCACTGCTGCTGCCCCTGGCCCTGCTGCTGCACGCCGCCAGG |
| | acid sequence | CCCGAGGTGCAGCTGGTGGAGTCCGGCGGCGGCGTGGTGCAGCCAGGCGGCTCTCTGAG |
| | | GCTGAGCTGCGCCGCCAGCGGCGACACCTTCGGAACGAAAGCCATGGGATGGTTCAGGC |
| | | AGGCCCCCGGCAAGGGACGCGAGTTTGTGGCCGCAATCACGTGGACAGCAGGCCTAACC |
| | | TACTACGCTGACTCCGTGAAGGGCAGGTTCACAATTTCTAAAGACCGCGCCAAGAACAC |
| | | CGTGGATCTGCAGATGAACTCCCTGCGCCCCGAGGACACAGCCCTGTACTACTGCGCCTC |
| | | ACGGCGCACGTTCTCATACGAGCACCCCAATTACTATACCAGGTCAGCCTACCACTTTTG |
| | | GGGACAGGGCACCCTGGTGACCGTGTCCTCCGCCGCCGCTATCGAGGTGATGTACCCTCC |
| | | CCCTTACCTGGACAACGAGAAATCAAACGGCACCATCATCCACGTGAAGGGAAAGCACC |
| | | TGTGTCCTAGCCCACTGTTCCCAGGCCCCAGCAAGCCCTTTTGGGTGCTGGTTGTGGTGG |
| | | GCGGCGTGCTGGCCTGCTACAGCCTGCTGGTCACCGTGGCCTTCATCATCTTCTGGAAGC |
| | | GGGGACGCAAGAAGCTGCTGTATATCTTTAAGCAACCATTCATGCGGCCCGTTCAAACG |
| | | ACTCAAGAAGAAGACGGGTGTAGCTGCCGGTTTCCCGAAGAAGAAGAGGGCGGCTGTGA |
| | | ACTACGCGTGAAGTTCTCCAGGAGCGCCGATGCCCCCGCCTACCAGCAGGGCCAGAACC |
| | | AGCTGTACAACGAGCTGAACCTGGGCCGGCGGGAGGAATACGACGTGCTGGACAAGCG |
| | | GAGAGGCCGGGACCCTGAGATGGGCGGAAAGCCTAGACGCAAGAACCCCCAGGAGGGC |
| | | CTGTACAACGAGCTGCAGAAAGATAAAATGGCCGAGGCCTATAGCGAGATCGGCATGAA |
| | | GGGCGAGAGAAGACGCGGCAAGGGGCACGATGGCCTGTACCAGGGGCTGAGCACCGCC |
| | | ACCAAAGACACCTACGATGCCCTGCACATGCAGGCCCTGCCCCCGCGGTAATAATAA |
| 49 | Anti-CD19 CAR #1 amino | MALPVTALLLPLALLLHAARPEVQLVESGGGVVQPGGSLRLSCAASGLFLSDYTLAWFRQ |
| | acid sequence with signal | APGKEREFVIVSTWNRYTYYAEGVKGRFTISRDNAKKTVYLQMNSLRPEDTALYYCAAKFRI |
| | peptide (underlined/bolded) | SYNVRDDSFYDLWGQGTLVTVSSAAAIEVMYPPPYLDNEKSNGTIIHVKGKHLCPSPLFPGPS |
| | | KPFWVLVVVGGVLACYSLLVTVAFIIFWVRSKRSRLLHSDYMNMTPRRPGPTRKHYQPYAP |
| | | PRDFAAYRSRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALHMQALPP |
| | | R |
| 50 | Anti-CD19 CAR #2 amino | MALPVTALLLPLALLLHAARPEVQLVESGGGVVQPGGSLRLSCAASGLFLSDYTLAWFRQ |
| | acid sequence with signal | APGKEREFVIVSTWNRYTYYAEGVKGRFTISRDNAKKTVYLQMNSLRPEDTALYYCAAKFRI |
| | peptide (underlined/bolded) | SYNVRDDSFYDLWGQGTLVTVSSAAAIEVMYPPPYLDNEKSNGTIIHVKGKHLCPSPLFPGPS |
| | | KPFWVLVVVGGVLACYSLLVTVAFIIFWKRGRKKLLYIFKQPFMRPVQTTQEEDGCSCRFPE |
| | | EEEGGCELRVKFSRSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDTYDALHMQALPPR |
| 51 | Anti-CD19 CAR #3 amino | MALPVTALLLPLALLLHAARPEVQLVESGGGVVQPGGSLRLSCAASGDTFGTKAMGWFR |
| | acid sequence with signal | QAPGKGREFVAAITWTAGLTYYADSVKGRFTISKDRAKNTVDLQMNSLRPEDTALYYCASR |
| | peptide (underlined/bolded) | RTFSYEHPNYYTRSAYHFWGQGTLVTVSSAAAIEVMYPPPYLDNEKSNGTIIHVKGKHLCPSP |
| | | LFPGPSKPFWVLVVVGGVLACYSLLVTVAFIIFWKRGRKKLLYIFKQPFMRPVQTTQEEDGCS |
| | | CRFPEEEEGGCELRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHMQ |
| | | ALPPR |
| 114 | CD8a-CD19-FMC63-CD28- | AUGGCUCUCCCAGUGACCGCCCUGCUGCUGCCCCUAGCCCUCCUUUUGCACGCAGCGA |
| | 40 CAR nucleic acid sequence | GGCCUGAGGUGAAGCUGCAGGAGUCCGGCCCCGGGCUGGUCGCGCCCAGCCAGAGCCU |
| | | CAGCGUGACCUGCACCGUGAGCGGCGUGAGCCUGCCCGACUACGGCGUCAGCUGGAUC |
| | | CGCCAGCCCCCCAGGAAGGGGCUGGAGUGGCUGGGCGUGAUCUGGGGCAGUGAGACG |
| | | ACCUAUUACAACUCCGCCCUCAAGAGCCGCCUAACCAUCAUCAAGGACAACAGCAAGA |
| | | GCCAGGUGUUCCUGAAGAUGAACAGCCUGCAGACCGACGACACCGCCAUCUACUACUG |
| | | CGCCAAGCACUACUACUACGGCGGGUCCUACGCCAUGGACUACUGGGGGCAGGGCACC |
| | | AGCGUGACCGUCAGCUCGGGCGGCGGCGGCAGCGGGGGAGGCGGGUCGGGGGGCGGC |
| | | GGCAGCGACAUCCAGAUGACCCAGACCACCAGCUCCCUGAGCGCCUCCCUCGGCGACC |
| | | GAGUCACGAUCAGCUGCAGGGCCUCCCAGGACAUCAGCAAGUACCUCAACUGGUACCA |
| | | GCAGAAGCCGGACGGGACCGUGAAGCUGCUGAUCUACCACACCUCCCGGCUGCACAGC |
| | | GGCGUGCCCAGCCGGUUCAGCGGCUCCGGCAGCGGCACCGACUACAGCCUGACCAUCA |
| | | GCAACCUGGAGCAGGAGGACAUCGCCACCUACUUCUGCCAGCAGGGGAACACCCUGCC |
| | | CUACACGUUCGGCGGCGGCACCAAGCUGGAGAUCAAGGCGGCCGCGAUAGAGGUAAU |
| | | GUACCCUCCUCCUUACCUGGACAACGAGAAGUCGAACGGCACCAUCAUCCACGUCAAG |
| | | GGCAAGCACCUCUGCCCCUCGCCCCUGUUCCCCGGCCCCUCCAAGCCCUUCUGGGUGCU |
| | | GGUCGUGGUUGGUGGCGUCCUCGCCUGCUACUCCCUCCUCGUCACGGUGGCGUUCAUC |
| | | AUCUUCUGGGUGCGGAGCAAGCGAAGCCGCCUCCUGCACAGCGACUACAUGAACAUGA |
| | | CGCCCCGCCGCCCCGGCCCGACCCGGAAGCACUACCAGCCCUACGCCCCGCCGCGCGAC |
| | | UUCGCGGCCUACCGCUCCCGGGUCAAGUUCUCCCGCAGCGCCGACGCCCCCGCGUACC |
| | | AGCAGGGCCAGAACCAGCUCUACAACGAGCUCAACCUGGGGAGGCGCGAGGAGUACGA |
| | | CGUGCUUGACAAGCGCCGUGGCCGGGACCCUGAGAUGGGGGGUAAGCCGCGACGGAA |
| | | GAACCCGCAGGAGGGCCUGUACAACGAGCUGCAGAAGGACAAGAUGGCGGAGGCCUA |
| | | CAGCGAGAUCGGGAUGAAGGGGGAGCGGCGGCGUGGCAAGGGGCACGACGGCCUGUA |
| | | CCAGGGCCUCAGUACGGCCACCAAGGACACCUACGACGCCUUGCACAUGCAGGCCCUC |
| | | CCCCCCCGCUAGUGAUGA |
| 122 | CD8a-CD19-FMC63-CD28- | MALPVTALLLPLALLLHAARPEVKLQESGPGLVAPSQSLSVTCTVSGVSLPDYGVSWIRQ |
| | 40 CAR amino acid sequence | PPRKGLEWLGVIWGSETTYYNSALKSRLTIIKDNSKSQVFLKMNSLQTDDTAIYYCAKHY |
| | | YYGGSYAMDYWGQGTSVTVSSGGGGSGGGGSGGGGSDIQMTQTTSSLSASLGDRVTISCR |
| | | ASQDISKYLNWYQQKPDGTVKLLIYHTSRLHSGVPSRFSGSGSGTDYSLTISNLEQEDIA |
| | | TYFCQQGNTLPYTFGGGTKLEIKAAAIEVMYPPPYLDNEKSNGTIIHVKGKHLCPSPLFP |
| | | GPSKPFWVLVVVGGVLACYSLLVTVAFIIFWVRSKRSRLLHSDYMNMTPRRPGPTRKHYQ |
| | | PYAPPRDFAAYRSRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLSTATKDTYDALHM |
| | | Q |
| | | ALPPR |
| 145 | CD8a-CD19-FMC63-CD28- | GGGUCAAGAUUAGAGAACGGUCGUAGCAUUAUCGGAGGUUCUGCCACCAUGGCUCUC |
| | 40 CAR nucleic acid sequence | CCAGUGACCGCCCUGCUGCUGCCCCUAGCCCUCCUUUUGCACGCAGCGAGGCCUGAGG |
| | | UGAAGCUGCAGGAGUCCGGCCCCGGGCUGGUCGCGCCCAGCCAGAGCCUCAGCGUGAC |
| | | CUGCACCGUGAGCGGCGUGAGCCUGCCCGACUACGGCGUCAGCUGGAUCCGCCAGCCC |
| | | CCCAGGAAGGGGCUGGAGUGGCUGGGCGUGAUCUGGGGCAGUGAGACGACCUAUUAC |
| | | AACUCCGCCCUCAAGAGCCGCCUAACCAUCAUCAAGGACAACAGCAAGAGCCAGGUGU |
| | | UCCUGAAGAUGAACAGCCUGCAGACCGACGACACCGCCAUCUACUACUGCGCCAAGCA |
| | | CUACUACUACGGCGGGUCCUACGCCAUGGACUACUGGGGGCAGGGCACCAGCGUGACC |
| | | GUCAGCUCGGGCGGCGGCGGCAGCGGGGGAGGCGGGUCGGGGGGCGGCGGCAGCGAC |
| | | AUCCAGAUGACCCAGACCACCAGCUCCCUGAGCGCCUCCCUCGGCGACCGAGUCACGA |
| | | UCAGCUGCAGGGCCUCCCAGGACAUCAGCAAGUACCUCAACUGGUACCAGCAGAAGCC |
| | | GGACGGGACCGUGAAGCUGCUGAUCUACCACACCUCCCGGCUGCACAGCGGCGUGCCC |
| | | AGCCGGUUCAGCGGCUCCGGCAGCGGCACCGACUACAGCCUGACCAUCAGCAACCUGG |
| | | AGCAGGAGGACAUCGCCACCUACUUCUGCCAGCAGGGGAACACCCUGCCCUACACGUU |
| | | CGGCGGCGGCACCAAGCUGGAGAUCAAGGCGGCCGCGAUAGAGGUAAUGUACCCUCCU |
| | | CCUUACCUGGACAACGAGAAGUCGAACGGCACCAUCAUCCACGUCAAGGGCAAGCACC |
| | | UCUGCCCCUCGCCCCUGUUCCCCGGCCCCUCCAAGCCCUUCUGGGUGCUGGUCGUGGU |
| | | UGGUGGCGUCCUCGCCUGCUACUCCCUCCUCGUCACGGUGGCGUUCAUCAUCUUCUGG |
| | | GUGCGGAGCAAGCGAAGCCGCCUCCUGCACAGCGACUACAUGAACAUGACGCCCCGCC |
| | | GCCCCGGCCCGACCCGGAAGCACUACCAGCCCUACGCCCCGCCGCGCGACUUCGCGGCC |
| | | UACCGCUCCCGGGUCAAGUUCUCCCGCAGCGCCGACGCCCCCGCGUACCAGCAGGGCC |
| | | AGAACCAGCUCUACAACGAGCUCAACCUGGGGAGGCGCGAGGAGUACGACGUGCUUG |
| | | ACAAGCGCCGUGGCCGGGACCCUGAGAUGGGGGGUAAGCCGCGACGGAAGAACCCGCA |
| | | GGAGGGCCUGUACAACGAGCUGCAGAAGGACAAGAUGGCGGAGGCCUACAGCGAGAU |
| | | CGGGAUGAAGGGGGAGCGGCGGCGUGGCAAGGGGCACGACGGCCUGUACCAGGGCCU |
| | | CAGUACGGCCACCAAGGACACCUACGACGCCUUGCACAUGCAGGCCCUCCCCCCCCGC |
| | | UAGUGAUGAGGCACAUUUGAAUAAAUUCUAUUACCAGUUCAAGCUUGGCGUAAUCAU |
| | | GGUCAUAGCUG |
| 149 | Human CD8a-Fc-HIS6 | SQFRVSPLDRTWNLGETVELKCQVLLSNPTSGCSWLFQPRGAAASPTFLLYLSQNKPKA |
| | | AEGLDTQRFSGKRLGDTFVLTLSDFRRENEGYYFCSALSNSIMYFSHFVPVFLPAKPTTT |
| | | PAPRPPTPAPTIASQPLSLRPEACRPAAGGAVHTRGLDFACPPCPAPELLGGPSVFLFPPKP |
| | | KDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRWQQGNVFSCSVMHEAL |
| | | HNHYTQKSLSLSPGHHHHHH |
| 150 | Human CD8a | SQFRVSPLDRTWNLGETVELKCQVLLSNPTSGCSWLFQPRGAAASPTFLLYLSQNKPKA |
| | | AEGLDTQRFSGKRLGDTFVLTLSDFRRENEGYYFCSALSNSIMYFSHFVPVFLPÅKPTTT |
| | | PAPRPPTPAPTIASQPLSLRPEACRPAAGGAVHTRGLDFAC |
| 151 | IFC | PPCPAPELLGGPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPVLDSDGSFFLYSKLTVD |
| | | KSRWQQGNVFSCSVMHEALHNHYTQKSLSLSPG |
| 152 | HIS6 | HHHHHH |
| 153 | Human CD8b-Fc-Twinstrep | LQQTPAYIKVQTNKMVMLSCEAKISLSNMRIYWLRQRQAPSSDSHHEFLALWDSAKGT |
| | | IHGEEVEQEKIAVFRDASRFILNLTSVKPEDSGIYFCMIVGSPELTFGKGTQLSVVDFLPT |
| | | TAQPTKKSTLKKRVCRLPRPETQKGPLCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVMHEALHNHYTQKSLSL |
| | | SPGWSHPQFEKGGGSGGGSGGSSAWSHPQFEK |
| 154 | Twinstrep | WSHPQFEKGGGGGGSGGSSAWSHPQFEK |
| 155 | Cynomolgus CD8a-Fc-HIS6 | NQFRVSPLGRTWNLGETVELKCQVLLSNPTSGCSWLFQPRGTAARPTFLLYLSQNKPK |
| | | AAEGLDTQRFSGKRLGDTFVLTLRDFRQENEGYYFCSALSNSIMYFSHFVPVFLPAKPTT |
| | | TPAPRPPTPAPTTASQPLSLRPEACRPAAGGSVNTRGLDFACPPCPAPELLGGPSVFLFPPK |
| | | PKDTLMISRTPEVTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPSRDELTKNQVSLTCLV |
| | | KGFYPSDIAVEWESNGQPENNYKTTPPVLDSDGSFFLYSKLTVDKSRWQQGNVFSCSVMHE |
| | | ALHNHYTQKSLSLSPGHHHHHH |
| 156 | Cynomolgus CD8a | NQFRVSPLGRTWNLGETVELKCQVLLSNPTSGCSWLFQPRGTAARPTFLLYLSQNKPK |
| | | AAEGLDTQRFSGKRLGDTFVLTLRDFRQENEGYYFCSALSNSIMYFSHFVPVFLPAKPTT |
| | | TPAPRPPTPAPTTASQPLSLRPEACRPAAGGSVNTRGLDFAC |
| 157 | Cynomolgus CD8b-Fc- | LQQTPAYIQVQTNKMAMLSCEAKISLGNMRIYWLRQRQAPSSDSHHEFLALWDSTKGT |
| | Twinstrep | VHGEEVAQEKIAVFRDASRFILNLTSVKPEDSGIYFCMIIGSPELTFGKGTQLSVVDFLPT |
| | | TAQPTKKSTPKKRGCRLPRPATQKGPLCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVMHEALHNHYTQKSLSL |
| | | SPGWSHPQFEKGGGSGGGSGGSSAWSHPQFEK |
| 158 | Cynomolgus CD8b | LQQTPAYIQVQTNKMAMLSCEAKISLGNMRIYWLRQRQAPSSDSHHEFLALWDSTKGT |
| | | VHGEEVAQEKIAVFRDASRFILNLTSVKPEDSGIYFCMIIGSPELTFGKGTQLSVVDFLPT |
| | | TAQPTKKSTPKKRGCRLPRPATQKGPLC |
| 160 | T0347015C01 (Abm CDR) | EVQLVESGGGLVQAGGSLRLSCAASGFTFDDYAIGWFRQAPGKEREEVSCIRTYDGNTYYID |
| | | SVKGRFTISSDNAKNTVYLQMNSLKPEDTAAYYCAAGSYYACAKYSRPDPSENHVDMDYW |
| | | GKGTLVTVSS |
| 161 | A044300805 | EVQLVESGGGVVQPGGSLRLSCAASGFTFEDYAIGWFRQAPGKEREEVSCIRTYDEQTYYAD |
| | (L11V, A14P, D30E, G55E, N5Q, | SVKGRFTISRDNAKNTVYLQMNSLRPEDTALYYCAAGSYYACAKYSRPDPSEGHVDLDYW |
| | I60A, S71R, K83R, A89L, | GQGTLVTVSS |
| | N100kG, M100oL, K105Q) | |
| 162 | A044300805_v1 (Abm CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVYLQMNSLRPEDTALYYCAAGSYYACAEYSRPDPSEGHVDLDYWG |
| | I60A, S71R, K83R, A89L, | QGTLVTVSS |
| | K100bE, N100KG, M100oL, K105Q) | |
| 163 | A044300805_v2 (Abm CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVYLQMNSLRPEDTALYYCAAGSYYACAYYSRPDPSEGHVDLDYW |
| | I60A, S71R, K83R, A89L, | GQGTLVTVSS |
| | K100bY, N100KG, M100oL, K105Q) | |
| 164 | A044300805_v3 (Abm CDR) | EVQLVESGGGVVQPGGSLRLSCAASGFTFEDYAIGWFRQAPGKEREEVSCIRTYDEQTYYAD |
| | (Abm CDR) | SVKGRFTISRDNAKNTVDLQMNSLRPEDTALYYCAAGSYYACAKYSRPDPSEGHVDLDYW |
| | (L11V, A14P, D30E, G55E, N56Q, | GQGTLVTVSS |
| | I60A, S71R, Y79D, K83R, | |
| | A89L, N100KG, M100oL, K105Q) | |
| 163 | A044300805_v4 (Abm CDR) | EVQLVESGGGVVQPGGSLRLSCAASGFTFEDYAIGWFRQAPGKEREEVSCIRTYDEQTYYAD |
| | (L11V,A14P,D30E,G55E,N5 | SVKGRFTISRDNAKNTVDLQMNSLRPEDTALYYCAAGSYYACAEYSRPDPSEGHVDLDYWG |
| | 6Q,I60A,S71R, Y79D,K83R | QGTLVTVSS |
| | A89L,K100bE,N100kG,M100 | |
| | oL,K105Q) | |
| 166 | A044300805_v5 (Abm CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVDLQMNSLRPEDTALYYCAAGSYYACAYYSRPDPSEGHVDLDYW |
| | I60A, S71R, Y79D, K83R, | GQGTLVTVSS |
| | A89L, K100bY, N100KG, M1000L, | |
| | K105Q) | |
| 167 | A044300805_v6 (Abm CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVSLQMNSLRPEDTALYYCAAGSYYACAKYSRPDPSEGHVDLDYWG |
| | I60A, S71R, Y79S, K83R, | QGTLVTVSS |
| | A89L, N100KG, M100oL, K105Q | |
| 168 | A044300805_v7 (Abm CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVSLQMNSLRPEDTALYYCAAGSYYACAEYSRPDPSEGHVDLDYWG |
| | I60A, S71R, Y79S, K83R, | QGTLVTVSS |
| | A89L, K100bE, N100KG, M100oL, | |
| | K105Q) | |
| 169 | A044300805_v8 (Abm CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVSLQMNSLRPEDTALYYCAAGSYYACAYYSRPDPSEGHVDLDYWG |
| | I60A, S71R, Y79S, K83R, A89L, | QGTLVTVSS |
| | K100bY, N100kG, M100oL, K105Q) | |
| 170 | T0347015C01 (Kabat CDR) | EVQLVESGGGLVQAGGSLRLSCAASGFTFDDYAIGWFRQAPGKEREEVSCIRTYDGNTYYID |
| | | SVKGRFTISSDNAKNTVYLQMNSLKPEDTAAYYCAAGSYYACAKYSRPDPSENHVDMDYW |
| | | GKGTLVTVSS |
| 171 | A044300805 (Kabat CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVYLQMNSLRPEDTALYYCAAGSYYACAKYSRPDPSEGHVDLDYW |
| | I60A, S71R, K83R, A89L, | GQGTLVTVSS |
| | N100kG, M100oL, K105Q) | |
| 172 | A044300805_v1 (Kabat CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVYLQMNSLRPEDTALYYCAAGSYYACAEYSRPDPSEGHVDLDYWG |
| | I60A, S71R, K83R, A89L, | QGTLVTVSS |
| | K100bE, N100KG, M100oL, K105Q | |
| 173 | A044300805_v2 (Kabat CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVYLQMNSLRPEDTALYYCAAGSYYACAYYSRPDPSEGHVDLDYW |
| | I60A, S71R, K83R, A89L, | GQGTLVTVSS |
| | K100bY, N100KG, M100oL, K105Q) | |
| 174 | A044300805_v3 (Kabat CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVDLQMNSLRPEDTALYYCAAGSYYACAKYSRPDPSEGHVDLDYW |
| | I60A, S71R, Y79D, K83R, | GQGTLVTVSS |
| | A89L, N100kG, M100oL, K105Q) | |
| 175 | A044300805_v4 (Kabat CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVDLQMNSLRPEDTALYYCAAGSYYACAEYSRPDPSEGHVDLDYWG |
| | 160A, S71R, Y79D, K83R, | QGTLVTVSS |
| | A89L, K100bE, N100kG, M100oL, | |
| | K105Q) | |
| 176 | A044300805_v5 (Kabat CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVDLQMNSLRPEDTALYYCAAGSYYACAYYSRPDPSEGHVDLDYW |
| | I60A, S71R, Y79D, K83R, | GQGTLVTVSS |
| | A89L, K100bY, N100KG, M1000L, | |
| | K105Q) | |
| 177 | A044300805_v6 (Kabat CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVSLQMNSLRPEDTALYYCAAGSYYACAKYSRPDPSEGHVDLDYWG |
| | I60A, S71R, Y79S, K83R, | QGTLVTVSS |
| | A89L, N100kG, M100oL, K105Q) | |
| 178 | A044300805_v7 (Kabat CDR) | EVQLVESGGGVVQPGGSLRLSCAASGFTFEDYAIGWFRQAPGKEREEVSCIRTYDEQTYYAD |
| | (L11V, A14P, D30E, G55E, N56Q, | SVKGRFTISRDNAKNTVSLQMNSLRPEDTALYYCAAGSYYACAEYSRPDPSEGHVDLDYWG |
| | I60A, S71R, Y79S, K83R, | QGTLVTVSS |
| | A89L, K100bE, N100KG, M100oL, | |
| | K105Q) | |
| 179 | A044300805_v8 (Kabat CDR) | EVQLVESGGGVVQPGGSLRLSCAASGFTFEDYAIGWFRQAPGKEREEVSCIRTYDEQTYYAD |
| | | SVKGRFTISRDNAKNTVSLQMNSLRPEDTALYYCAAGSYYACAYYSRPDPSEGHVDLDYWG |
| | | QGTLVTVSS |
| 180 | (L11V, A14P, D30E, G35E, N56Q, | EVQLVESGGGLVQAGGSLRLSCAAS |
| | I60A, S71R, Y79S, K83R, | |
| | A89L, K100bY, N100kG, M100oL, | |
| | K105Q) | |
| 187 | A044300805 (Abm | EVQLVESGGGVVQPGGSLRLSCAAS |
| | CDR)_fw1 | |
| 194 | A044300805_v1 (Abm | EVQLVESGGGVVQPGGSLRLSCAAS |
| | CDR)_fw1 | |
| 201 | A044300805_v2 (Abm | EVQLVESGGGVVQPGGSLRLSCAAS |
| | CDR)_fw1 | |
| 208 | A044300805_v3 (Abm | EVQLVESGGGVVQPGGSLRLSCAAS |
| | CDR)_fw1 | |
| 215 | A044300805_v4 (Abm | EVQLVESGGGVVQPGGSLRLSCAAS |
| | CDR)_fw1 | |
| 222 | A044300805_v5 (Abm | EVQLVESGGGVVQPGGSLRLSCAAS |
| | CDR)_fw1 | |
| 229 | A044300805_v6 (Abm | EVQLVESGGGVVQPGGSLRLSCAAS |
| | CDR)_fw1 | |
| 236 | A044300805_v7 (Abm | EVQLVESGGGVVQPGGSLRLSCAAS |
| | CDR)_fw1 | |
| 243 | A044300805_v8 (Abm | EVQLVESGGGVVQPGGSLRLSCAAS |
| | CDR)_fw1 | |
| 181 | T0347015C01 (Abm | GFTFDDYAIG |
| | CDR)_CDR1 | |
| 188 | A044300805 (Abm | GFTFEDYAIG |
| | CDR)_CDR1 | |
| 195 | A044300805_v1 (Abm | GFTFEDYAIG |
| | CDR)_CDR1 | |
| 202 | A044300805_v2 (Abm | GFTFEDYAIG |
| | CDR)_CDR1 | |
| 209 | A044300805_v3 (Abm | GFTFEDYAIG |
| | CDR)_CDR1 | |
| 216 | A044300805_v3 (Abm | GFTFEDYAIG |
| | CDR)_CDR1 | |
| 223 | A044300805_v3 (Abm | GFTFEDYAIG |
| | CDR)_CDR1 | |
| 230 | A044300805_v3 (Abm | GFTFEDYAIG |
| | CDR)_CDR1 | |
| 237 | A044300805_v3 (Abm | GFTFEDYAIG |
| | CDR)_CDR1 | |
| 244 | A044300805_v3 (Abm | GFTFEDYAIG |
| | CDR)_CDR1 | |
| 182 | T0347015C01 (Abm | WFRQAPGKEREEVS |
| | CDR)_fw2 | |
| 189 | A044300805 (Abm | WFRQAPGKEREEVS |
| | CDR)_fw2 | |
| 196 | A044300805_v1 (Abm | WFRQAPGKEREEVS |
| | CDR)_fw2 | |
| 203 | A044300805_v2 (Abm | WFRQAPGKEREEVS |
| | CDR)_fw2 | |
| 210 | A044300805_v3 (Abm | WFRQAPGKEREEVS |
| | CDR)_fw2 | |
| 217 | A044300805_v4 (Abm | WFRQAPGKEREEVS |
| | CDR)_fw2 | |
| 224 | A044300805_v5 (Abm | WFRQAPGKEREEVS |
| | CDR)_fw2 | |
| 231 | A044300805_v6 (Abm | WFRQAPGKEREEVS |
| | CDR)_fw2 | |
| 238 | A044300805_v7 (Abm | WFRQAPGKEREEVS |
| | CDR)_fw2 | |
| 245 | A044300805_v8 (Abm | WFRQAPGKEREEVS |
| | CDR)_fw2 | |
| 183 | T0347015C01 (Abm | CIRTYDGNTY |
| | CDR)_CDR2 | |
| 190 | A044300805 (Abm | CIRTYDEQTY |
| | CDR)_CDR2 | |
| 197 | A044300805_v1 (Abm | CIRTYDEQTY |
| | CDR)_CDR2 | |
| 204 | A044300805_v2 (Abm | CIRTYDEQTY |
| | CDR)_CDR2 | |
| 211 | A044300805_v3 (Abm | CIRTYDEQTY |
| | CDR)_CDR2 | |
| 218 | A044300805_v3 (Abm | CIRTYDEQTY |
| | CDR)_CDR2 | |
| 225 | A044300805_v3 (Abm | CIRTYDEQTY |
| | CDR)_CDR2 | |
| 232 | A044300805_v3 (Abm | CIRTYDEQTY |
| | CDR)_CDR2 | |
| 239 | A044300805_v3 (Abm | CIRTYDEQTY |
| | CDR)_CDR2 | |
| 246 | A044300805_v3 (Abm | CIRTYDEQTY |
| | CDR)_CDR2 | |
| 184 | T0347015C01 (Abm | YIDSVKGRFTISSDNAKNTVYLQMNSLKPEDTAAYYCAA |
| | CDR)_fw3 | |
| 191 | A044300805 (Abm | YADSVKGRFTISRDNAKNTVYLQMNSLRPEDTALYYCAA |
| | CDR)_fw3 | |
| 198 | A044300805_v1 (Abm | YADSVKGRFTISRDNAKNTVYLQMNSLRPEDTALYYCAA |
| | CDR)_fw3 | |
| 205 | A044300805_v2 (Abm | YADSVKGRFTISRDNAKNTVYLQMNSLRPEDTALYYCAA |
| | CDR)_fw3 | |
| 212 | A044300805_v3 (Abm | YADSVKGRFTISRDNAKNTVDLQMNSLRPEDTALYYCAA |
| | CDR)_fw3 | |
| 219 | A044300805_v4 (Abm | YADSVKGRFTISRDNAKNTVDLQMNSLRPEDTALYYCAA |
| | CDR)_fw3 | |
| 226 | A044300805_v5 (Abm | YADSVKGRFTISRDNAKNTVDLQMNSLRPEDTALYYCAA |
| | CDR)_fw3 | |
| 233 | A044300805_v6 (Abm | YADSVKGRFTISRDNAKNTVSLQMNSLRPEDTALYYCAA |
| | CDR)_fw3 | |
| 240 | A044300805_v7 (Abm | YADSVKGRFTISRDNAKNTVSLQMNSLRPEDTALYYCAA |
| | CDR)_fw3 | |
| 247 | A044300805_v8 (Abm | YADSVKGRFTISRDNAKNTVSLQMNSLRPEDTALYYCAA |
| | CDR)_fw3 | |
| 185 | T0347015C01 (Abm | GSYYACAKYSRPDPSENHVDMDY |
| | CDR)_CDR3 | |
| 192 | A044300805 (Abm | GSYYACAKYSRPDPSEGHVDLDY |
| | CDR)_CDR3 | |
| 199 | A044300805_v1 (Abm | GSYYACAEYSRPDPSEGHVDLDY |
| | CDR)_CDR3 | |
| 206 | A044300805_v2 (Abm | GSYYACAYYSRPDPSEGHVDLDY |
| | CDR)_CDR3 | |
| 213 | A044300805_v3 (Abm | GSYYACAKYSRPDPSEGHVDLDY |
| | CDR)_CDR3 | |
| 220 | A044300805_v3 (Abm | GSYYACAEYSRPDPSEGHVDLDY |
| | CDR)_CDR3 | |
| 227 | A044300805_v3 (Abm | GSYYACAYYSRPDPSEGHVDLDY |
| | CDR)_CDR3 | |
| 234 | A044300805_v3 (Abm | GSYYACAKYSRPDPSEGHVDLDY |
| | CDR)_CDR3 | |
| 241 | A044300805_v3 (Abm | GSYYACAEYSRPDPSEGHVDLDY |
| | CDR)_CDR3 | |
| 248 | A044300805_v3 (Abm | GSYYACAYYSRPDPSEGHVDLDY |
| | CDR)_CDR3 | |
| 186 | T0347015C01 (Abm | WGKGTLVTVSS |
| | CDR)_fw4 | |
| 193 | A044300805 (Abm | WGQGTLVTVSS |
| | CDR)_fw4 | |
| 200 | A044300805_v1 (Abm | WGQGTLVTVSS |
| | CDR)_fw4 | |
| 207 | A044300805_v2 (Abm | WGQGTLVTVSS |
| | CDR)_fw4 | |
| 214 | A044300805_v3 (Abm | WGQGTLVTVSS |
| | CDR)_fw4 | |
| 221 | A044300805_v4 (Abm | WGQGTLVTVSS |
| | CDR)_fw4 | |
| 228 | A044300805_v5 (Abm | WGQGTLVTVSS |
| | CDR)_fw4 | |
| 235 | A044300805_v6 (Abm | WGQGTLVTVSS |
| | CDR)_fw4 | |
| 242 | A044300805_v7 (Abm | WGQGTLVTVSS |
| | CDR)_fw4 | |
| 249 | A044300805_v8 (Abm | WGQGTLVTVSS |
| | CDR)_fw4 | |
| 250 | T0347015C01 (Kabat | EVQLVESGGGLVQAGGSLRLSCAASGFTFD |
| | CDR)_fw1 | |
| 257 | A044300805 (Kabat | EVQLVESGGGVVQPGGSLRLSCAASGFTFE |
| | CDR)_fw1 | |
| 264 | A044300805_v1 (Kabat | EVQLVESGGGVVQPGGSLRLSCAASGFTFE |
| | CDR)_fw1 | |
| 271 | A044300805_v2 (Kabat | EVQLVESGGGVVQPGGSLRLSCAASGFTFE |
| | CDR)_fw1 | |
| 278 | A044300805_v3 (Kabat | EVQLVESGGGVVQPGGSLRLSCAASGFTFE |
| | CDR)_fw1 | |
| 285 | A044300805_v4 (Kabat | EVQLVESGGGVVQPGGSLRLSCAASGFTFE |
| | CDR)_fw1 | |
| 292 | A044300805_v5 (Kabat | EVQLVESGGGVVQPGGSLRLSCAASGFTFE |
| | CDR)_fw1 | |
| 299 | A044300805_v6 (Kabat | EVQLVESGGGVVQPGGSLRLSCAASGFTFE |
| | CDR)_fw1 | |
| 306 | A044300805_v7 (Kabat | EVQLVESGGGVVQPGGSLRLSCAASGFTFE |
| | CDR)_fw1 | |
| 313 | A044300805_v8 (Kabat | EVQLVESGGGVVQPGGSLRLSCAASGFTFE |
| | CDR)_fw1 | |
| 251 | T0347015C01 (Kabat | DYAIG |
| | CDR)_CDR1 | |
| 258 | A044300805 (Kabat | DYAIG |
| | CDR)_CDR1 | |
| 265 | A044300805_v1 (Kabat | DYAIG |
| | CDR)_CDR1 | |
| 272 | A044300805_v2 (Kabat | DYAIG |
| | CDR)_CDR1 | |
| 279 | A044300805_v3 (Kabat | DYAIG |
| | CDR)_CDR1 | |
| 286 | A044300805_v3 (Kabat | DYAIG |
| | CDR)_CDR1 | |
| 293 | A044300805_v3 (Kabat | DYAIG |
| | CDR)_CDR1 | |
| 300 | A044300805_v3 (Kabat | DYAIG |
| | CDR)_CDR1 | |
| 307 | A044300805_v3 (Kabat | DYAIG |
| | CDR)_CDR1 | |
| 314 | A044300805_v3 (Kabat | DYAIG |
| | CDR)_CDR1 | |
| 252 | T0347015C01 (Kabat | WFRQAPGKEREEVS |
| | CDR)_fw2 | |
| 259 | A044300805 (Kabat | WFRQAPGKEREEVS |
| | CDR)_fw2 | |
| 266 | A044300805_v1 (Kabat | WFRQAPGKEREEVS |
| | CDR)_fw2 | |
| 273 | A044300805_v2 (Kabat | WFRQAPGKEREEVS |
| | CDR)_fw2 | |
| 280 | A044300805_v3 (Kabat | WFRQAPGKEREEVS |
| | CDR)_fw2 | |
| 287 | A044300805_v4 (Kabat | WFRQAPGKEREEVS |
| | CDR)_fw2 | |
| 294 | A044300805_v5 (Kabat | WFRQAPGKEREEVS |
| | CDR)_fw2 | |
| 301 | A044300805_v6 (Kabat | WFRQAPGKEREEVS |
| | CDR)_fw2 | |
| 308 | A044300805_v7 (Kabat | WFRQAPGKEREEVS |
| | CDR)_fw2 | |
| 315 | A044300805_v8 (Kabat | WFRQAPGKEREEVS |
| | CDR)_fw2 | |
| 253 | T0347015C01 (Kabat | CIRTYDGNTYYIDSVKG |
| | CDR)_CDR2 | |
| 260 | A044300805 (Kabat | CIRTYDEQTYYADSVKG |
| | CDR)_CDR2 | |
| 267 | A044300805_v1 (Kabat | CIRTYDEQTYYADSVKG |
| | CDR)_CDR2 | |
| 274 | A044300805_v2 (Kabat | CIRTYDEQTYYADSVKG |
| | CDR)_CDR2 | |
| 281 | A044300805_v3 (Kabat | CIRTYDEQTYYADSVKG |
| | CDR)_CDR2 | |
| 288 | A044300805_v3 (Kabat | CIRTYDEQTYYADSVKG |
| | CDR)_CDR2 | |
| 295 | A044300805_v3 (Kabat | CIRTYDEQTYYADSVKG |
| | CDR)_CDR2 | |
| 302 | A044300805_v3 (Kabat | CIRTYDEQTYYADSVKG |
| | CDR)_CDR2 | |
| 309 | A044300805_v3 (Kabat | CIRTYDEQTYYADSVKG |
| | CDR)_CDR2 | |
| 316 | A044300805_v3 (Kabat | YADSVKGCIRTYDEQTY |
| | CDR)_CDR2 | |
| 254 | T0347015C01 (Kabat | RFTISSDNAKNTVYLQMNSLKPEDTAAYYCAA |
| | CDR)_fw3 | |
| 261 | A044300805 (Kabat | RFTISRDNAKNTVYLQMNSLRPEDTALYYCAA |
| | CDR)_fw3 | |
| 268 | A044300805_v1 (Kabat | RFTISRDNAKNTVYLQMNSLRPEDTALYYCAA |
| | CDR)_fw3 | |
| 275 | A044300805_v2 (Kabat | RFTISRDNAKNTVYLQMNSLRPEDTALYYCAA |
| | CDR)_fw3 | |
| 282 | A044300805_v3 (Kabat | RFTISRDNAKNTVDLQMNSLRPEDTALYYCAA |
| | CDR)_fw3 | |
| 289 | A044300805_v4 (Kabat | RFTISRDNAKNTVDLQMNSLRPEDTALYYCAA |
| | CDR)_fw3 | |
| 296 | A044300805_v5 (Kabat | RFTISRDNAKNTVDLQMNSLRPEDTALYYCAA |
| | CDR)_fw3 | |
| 303 | A044300805_v6 (Kabat | RFTISRDNAKNTVSLQMNSLRPEDTALYYCAA |
| | CDR)_fw3 | |
| 310 | A044300805_v7 (Kabat | RFTISRDNAKNTVSLQMNSLRPEDTALYYCAA |
| | CDR)_fw3 | |
| 317 | A044300805_v8 (Kabat | RFTISRDNAKNTVSLQMNSLRPEDTALYYCAA |
| | CDR)_fw3 | |
| 255 | T0347015C01 (Kabat | GSYYACAKYSRPDPSENHVDMDY |
| | CDR)_CDR3 | |
| 262 | A044300805 (Kabat | GSYYACAKYSRPDPSEGHVDLDY |
| | CDR)_CDR3 | |
| 269 | A044300805_v1 (Kabat | GSYYACAEYSRPDPSEGHVDLDY |
| | CDR)_CDR3 | |
| 276 | A044300805_v2 (Kabat | GSYYACAYYSRPDPSEGHVDLDY |
| | CDR)_CDR3 | |
| 283 | A044300805_v3 (Kabat | GSYYACAKYSRPDPSEGHVDLDY |
| | CDR)_CDR3 | |
| 290 | A044300805_v3 (Kabat | GSYYACAEYSRPDPSEGHVDLDY |
| | CDR)_CDR3 | |
| 297 | A044300805_v3 (Kabat | GSYYACAYYSRPDPSEGHVDLDY |
| | CDR)_CDR3 | |
| 304 | A044300805_v3 (Kabat | GSYYACAKYSRPDPSEGHVDLDY |
| | CDR)_CDR3 | |
| 311 | A044300805_v3 (Kabat | GSYYACAEYSRPDPSEGHVDLDY |
| | CDR)_CDR3 | |
| 318 | A044300805_v3 (Kabat | GSYYACAYYSRPDPSEGHVDLDY |
| | CDR)_CDR3 | |
| 256 | T0347015C01 (Kabat | WGKGTLVTVSS |
| | CDR)_fw4 | |
| 263 | A044300805 (Kabat | WGQGTLVTVSS |
| | CDR)_fw4 | |
| 270 | A044300805_v1 (Kabat | WGQGTLVTVSS |
| | CDR)_fw4 | |
| 277 | A044300805_v2 (Kabat | WGQGTLVTVSS |
| | CDR)_fw4 | |
| 284 | A044300805_v3 (Kabat | WGQGTLVTVSS |
| | CDR)_fw4 | |
| 291 | A044300805_v4 (Kabat | WGQGTLVTVSS |
| | CDR)_fw4 | |
| 298 | A044300805_v5 (Kabat | WGQGTLVTVSS |
| | CDR)_fw4 | |
| 305 | A044300805_v6 (Kabat | WGQGTLVTVSS |
| | CDR)_fw4 | |
| 312 | A044300805_v7 (Kabat | WGQGTLVTVSS |
| | CDR)_fw4 | |
| 319 | A044300805_v8 (Kabat | WGQGTLVTVSS |
| | CDR)_fw4 | |
| 320 | Alb8 | EVQLVESGGGLVQPGNSLRLSCAASGFTFSSFGMSWVRQAPGKGLEWVSSISGSGSDTLYAD |
| | | SVKGRFTISRDNAKTTLYLQMNSLRPEDTAVYYCTIGGSLSRSSQGTLVTVSS |
| 321 | Alb23 | EVQLLESGGGLVQPGGSLRLSCAASGFTFRSFGMSWVRQAPGKGPEWVSSISGSGSDTLYAD |
| | | SVKGRFTISRDNSKNTLYLQMNSLRPEDTAVYYCTIGGSLSRSSQGTLVTVSS |
| 322 | Alb129 | EVQLVESGGGVVQPGNSLRLSCAASGFTFSSFGMSWVRQAPGKGLEWVSSISGSGSDTLYAD |
| | | SVKGRFTISRDNAKTTLYLQMNSLRPEDTATYYCTIGGSLSRSSQGTLVTVSSA |
| 323 | Alb132 | EVQLVESGGGVVQPGGSLRLSCAASGFTFRSFGMSWVRQAPGKGPEWVSSISGSGSDTLYAD |
| | | SVKGRFTISRDNSKNTLYLQMNSLRPEDTATYYCTIGGSLSRSSQGTLVTVSSA |
| 324 | Alb11 | EVQLVESGGGLVQPGNSLRLSCAASGFTFSSFGMSWVRQAPGKGLEWVSSISGSGSDTLYAD |
| | | SVKGRFTISRDNAKTTLYLQMNSLRPEDTAVYYCTIGGSLSRSSQGTLVTVSS |
| 325 | Alb11 (S112K)-A | EVQLVESGGGLVQPGNSLRLSCAASGFTFSSFGMSWVRQAPGKGLEWVSSISGSGSDTLYAD |
| | | SVKGRFTISRDNAKTTLYLQMNSLRPEDTAVYYCTIGGSLSRSSQGTLVKVSSA |
| 326 | Alb82 | EVQLVESGGGVVQPGNSLRLSCAASGFTFSSFGMSWVRQAPGKGLEWVSSISGSGSDTLYAD |
| | | SVKGRFTISRDNAKTTLYLQMNSLRPEDTALYYCTIGGSLSRSSQGTLVTVSS |
| 327 | Alb82-A | EVQLVESGGGVVQPGNSLRLSCAASGFTFSSFGMSWVRQAPGKGLEWVSSISGSGSDTLYAD |
| | | SVKGRFTISRDNAKTTLYLQMNSLRPEDTALYYCTIGGSLSRSSQGTLVTVSSA |
| 328 | Alb82-AA | EVQLVESGGGVVQPGNSLRLSCAASGFTFSSFGMSWVRQAPGKGLEWVSSISGSGSDTLYAD |
| | | SVKGRFTISRDNAKTTLYLQMNSLRPEDTALYYCTIGGSLSRSSQGTLVTVSSAA |
| 329 | Alb82-AAA | EVQLVESGGGVVQPGNSLRLSCAASGFTFSSFGMSWVRQAPGKGLEWVSSISGSGSDTLYAD |
| | | SVKGRFTISRDNAKTTLYLQMNSLRPEDTALYYCTIGGSLSRSSQGTLVTVSSAAA |
| 330 | Alb82-G | EVQLVESGGGVVQPGNSLRLSCAASGFTFSSFGMSWVRQAPGKGLEWVSSISGSGSDTLYAD |
| | | SVKGRFTISRDNAKTTLYLQMNSLRPEDTALYYCTIGGSLSRSSQGTLVTVSSG |
| 331 | Alb82-GG | EVQLVESGGGVVQPGNSLRLSCAASGFTFSSFGMSWVRQAPGKGLEWVSSISGSGSDTLYAD |
| | | SVKGRFTISRDNAKTTLYLQMNSLRPEDTALYYCTIGGSLSRSSQGTLVTVSSGG |
| 332 | Alb82-GGG | EVQLVESGGGVVQPGNSLRLSCAASGFTFSSFGMSWVRQAPGKGLEWVSSISGSGSDTLYAD |
| | | SVKGRFTISRDNAKTTLYLQMNSLRPEDTALYYCTIGGSLSRSSQGTLVTVSSGGG |
| 333 | Alb223 | EVQLVESGGGVVQPGGSLRLSCAASGFTFRSFGMSWVRQAPGKGPEWVSSISGSGSDTLYAD |
| | | SVKGRFTISRDNSKNTLYLQMNSLRPEDTALYYCTIGGSLSRSSQGTLVTVSSA |
| 334 | AlbX00001 | EVQLVESGGGVVQPGGSLRLSCAASGLTFSSYAMGWFRQAPGKERERVVSISRGGGYTYYA |
| | | DSVKGRFTISRDNSENTVYLQMNSLRPEDTALYYCAAARYWATGSEYEFDYWGQGTLVTVS |
| | | S |
| 335 | Alb23002 | EVQLVESGGGVVQPGGSLRLSCAASGFTFRSFGMSWVRQAPGKGPEWVSSISGSGSDTLYAD |
| | | SVKGRFTISRDNSKNTLYLQMNSLRPEDTALYYCTIGGSLSRSSQGTLVTVSS |
| 336 | 3A linker | AAA |
| 337 | 5GS linker | GGGGS |
| 338 | 7GS linker | SGGSGGS |
| 339 | 8GS linker | GGGGSGGS |
| 340 | 9GS Tinker | GGGGSGGGS |
| 341 | 10GS linker | GGGGSGGGGS |
| 342 | 13GS linker | GGGGSGGGGSGGGGS |
| 343 | 18GS linker | GGGGSGGGGSGGGGSGGS |
| 344 | 20GS Tinker | GGGGSGGGGGGGGSGGGGS |
| 345 | 25GS Tinker | GGGGSGGGGSGGGGSGGGGGGGGS |
| 346 | 30GS linker | GGGGSGGGGSGGGGSGGGGSGGGGSGGGGS |
| 347 | 35GS linker | GGGGSGGGGSGGGGSGGGGSGGGGSGGGGSGGGGS |
| 348 | 40GS linker | GGGGSGGGGSGGGGSGGGGSGGGGSGGGGSGGGGSGGGGS |
| 349 | G1 hinge | EPKSCDKTHTCPPCP |
| 350 | 9GS-G1 hinge | GGGGSGGGSEPKSCDKTHTCPPCP |
| 351 | Llama upper long hinge | EPKTPKPQPAAA |
| | region | |
| 352 | G3 hinge | ELKTPLGDTTHTCPRCPEPKSCDTPPPCPRCPEPKSCDTPPPCPRCPEPKSCDTPPPCPRCP |
| 353 | | VTVSS(X)n |
| 354 | | VKVSS |
| 355 | | VQVSS |
| 356 | | VTVKS |
| 357 | | VTVQS |
| 358 | | VKVKS |
| 359 | | VKVQS |
| 360 | | VQVKS |
| 361 | | VQVQS |
| 362 | | VTVSSA |
| 363 | | VKVSSA |
| 364 | | VQVSSA |
| 365 | | VTVKSA |
| 366 | | VTVQSA |
| 367 | | VKVKSA |
| 368 | | VKVQSA |
| 369 | | VQVKSA |
| 370 | | VQVQSA |
| 371 | | VTVSS |
| 372 | | KERE |
| 373 | | KQRE |
| 374 | | GLEW |
| 375 | | KEREL |
| 376 | | KEREF |
| 377 | | KQREL |
| 378 | | KQREF |
| 379 | | KEREG |
| 380 | | KQREW |
| 381 | | KQREG |
| 382 | | TERE |
| 383 | | TEREL |
| 384 | | TQRE |
| 385 | | TQREL |
| 386 | | KECE |
| 387 | | KECEL |
| 388 | | KECER |
| 389 | | KQCE |
| 390 | | KQCEL |
| 391 | | RERE |
| 392 | | REREG |
| 393 | | RQRE |
| 394 | | RQREL |
| 395 | | RQREF |
| 396 | | RQREW |
| 397 | | QERE |
| 398 | | QEREG |
| 399 | | QQRE |
| 400 | | QQREW |
| 401 | | QQREL |
| 402 | | QQREF |
| 403 | | KGRE |
| 404 | | KGREG |
| 405 | | KDRE |
| 406 | | KDREV |
| 407 | | DECKL |
| 408 | | NVCEL |
| 409 | | GVEW |
| 410 | | EPEW |
| 411 | | GLER |
| 412 | | DQEW |
| 413 | | DLEW |
| 414 | | GIEW |
| 415 | | ELEW |
| 416 | | GPEW |
| 417 | | EWLP |
| 418 | | GPER |
| 419 | BDSn ISVD | EVQLVESGGGLVQAGGSLRLSCAASGSTFSDYGVGWFRQAPGKGREFVADIDWNGEHTSYA |
| | | DSVKGRFATSRDNAKNTAYLQMNSLKPEDTAVYYCAADALPYTVRKYNYWGQGTQVTVS |
| | | SGGCGGHHHHHH |
| 420 | BDSn ISVD_fw1 | EVQLVESGGGLVQAGGSLRLSCAAS |
| 421 | BDSn ISVD_CDR1 | GSTFSDYG |
| 422 | BDSn ISVD_fw2 | VGWFRQAPGKGREFVAD |
| 423 | BDSn ISVD_CDR2 | IDWNGEHT |
| 424 | BDSn ISVD_fw3 | SYADSVKGRFATSRDNAKNTAYLQMNSLKPEDTAVYYC |
| 425 | TBDSn ISVD_CDR3 | AADALPYTVRKYNY |
| 426 | BDSn ISVD_fw4 | WGQGTQVTVSSGGCGGHHHHHH |
| 427 | anti-CD19-A H-CDR1 (Kabat | DYTLA |
| | numbering) | |
| 428 | anti-CD19-A H-CDR2 (Kabat | VSTWNRYTYYAEGVKG |
| | numbering) | |
| 429 | anti-CD19-A H-CDR3 (Kabat | KFRISYNVRDDSFYDL |
| | numbering) | |
| 430 | GM-CSF signal peptide | MWLQNLLLLGAVVCSIS |
| 431 | CD28 hinge-transmembrane | IEVMYPPPYLDNEKSNGTIIHVKGKHLCPSPLFPGPSKPFWVLVVVGGVLACYSLLVTVAFIIF |
| | domain | WV |
| 432 | CD137 (4-1BB) co-stim | RGRKKLLYIFKQPFMRPVQTTQEEDGCSCRFPEEEEGGCEL |
| 433 | VHH-A (VHH of CAR #1, | EVQLVESGGGVVQPGGSLRLSCAASGLFLSDYTLAWFRQAPGKEREFVIVSTWNRYTYYAE |
| | CAR #2, CAR #41 and CAR | GVKGRFTISRDNAKKTVYLQMNSLRPEDTALYYCAAKFRISYNVRDDSFYDLWGQGTLVTV |
| | #42) | SS |
| 434 | CD28 hinge-transmembrane | IEVMYPPPYLDNEKSNGTIIHVKGKHLCPSPLFPGPSKPFWVLVVVGGVLACYSLLVTVAFIIF |
| | domain | WK |
| 435 | Anti-CD19 CAR#1 | EVQLVESGGGVVQPGGSLRLSCAASGLFLSDYTLAWFRQAPGKEREFVIVSTWNRYTYYAE |
| | | GVKGRFTISRDNAKKTVYLQMNSLRPEDTALYYCAAKFRISYNVRDDSFYDLWGQGTLVTV |
| | | SSAAAIEVMYPPPYLDNEKSNGTIIHVKGKHLCPSPLFPGPSKPFWVLVVVGGVLACYSLLVT |
| | | VAFIIFWVRSKRSRLLHSDYMNMTPRRPGPTRKHYQPYAPPRDFAAYRSRVKFSRSADAPAY |
| | | QQGQNQLYNELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 436 | Anti-CD19 CAR#2 | EVQLVESGGGVVQPGGSLRLSCAASGLFLSDYTLAWFRQAPGKEREFVIVSTWNRYTYYAE |
| | | GVKGRFTISRDNAKKTVYLQMNSLRPEDTALYYCAAKFRISYNVRDDSFYDLWGQGTLVTV |
| | | SSAAAIEVMYPPPYLDNEKSNGTIIHVKGKHLCPSPLFPGPSKPFWVLVVVGGVLACYSLLVT |
| | | VAFIIFWKRGRKKLLYIFKQPFMRPVQTTQEEDGCSCRFPEEEEGGCELRVKFSRSADAPAYQ |
| | | QGQNQLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 437 | Anti-CD19 CAR#3 | EVQLVESGGGVVQPGGSLRLSCAASGDTFGTKAMGWFRQAPGKGREFVAAITWTAGLTYY |
| | | ADSVKGRFTISKDRAKNTVDLQMNSLRPEDTALYYCASRRTFSYEHPNYYTRSAYHFWGQG |
| | | TLVTVSSAAAIEVMYPPPYLDNEKSNGTIIHVKGKHLCPSPLFPGPSKPFWVLVVVGGVLACY |
| | | SLLVTVAFIIFWKRGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 438 | CD28 hinge #1 | IEVMYPPPYLDNEKSNGTIIHVKGKHLCPSPLFPGPSKPF |
| 439 | CD28 hinge #2 | IEVMYPPPYLDNEKSNGTIIHVKGKHLCPSPLFPGPSKPF |
| 440 | CD28 transmembrane domain | WVLVVVGGVLACYSLLVTVAFIIFWK |
| | #1 | |
| 441 | Anti-CD19 CAR #4 nucleic | ATGGAGACCGACACCCTCCTGCTGTGGGTGCTGCTGCTTTGGGTGCCCGGGAGCACGGG |
| | acid sequence | GGAGGTGAAGCTGCAGGAGTCCGGCCCCGGGCTGGTCGCGCCCAGCCAGAGCCTCAGCG |
| | | TGACCTGCACCGTGAGCGGCGTGAGCCTGCCCGACTACGGCGTCAGCTGGATCCGCCAG |
| | | CCCCCCAGGAAGGGGCTGGAGTGGCTGGGCGTGATCTGGGGCAGTGAGACGACCTATTA |
| | | CAACTCCGCCCTCAAGAGCCGCCTAACCATCATCAAGGACAACAGCAAGAGCCAGGTGT |
| | | TCCTGAAGATGAACAGCCTGCAGACCGACGACACCGCCATCTACTACTGCGCCAAGCAC |
| | | TACTACTACGGCGGGTCCTACGCCATGGACTACTGGGGGCAGGGCACCAGCGTGACCGT |
| | | CAGCTCGGGCGGCGGCGGCAGCGGGGGAGGCGGGTCGGGGGGCGGCGGCAGCGACATC |
| | | CAGATGACCCAGACCACCAGCTCCCTGAGCGCCTCCCTCGGCGACCGAGTCACGATCAG |
| | | CTGCAGGGCCTCCCAGGACATCAGCAAGTACCTCAACTGGTACCAGCAGAAGCCGGACG |
| | | GGACCGTGAAGCTGCTGATCTACCACACCTCCCGGCTGCACAGCGGCGTGCCCAGCCGG |
| | | TTCAGCGGCTCCGGCAGCGGCACCGACTACAGCCTGACCATCAGCAACCTGGAGCAGGA |
| | | GGACATCGCCACCTACTTCTGCCAGCAGGGGAACACCCTGCCCTACACGTTCGGCGGCG |
| | | GCACCAAGCTGGAGATCAAGGCGGCCGCGATAGAGGTAATGTACCCTCCTCCTTACCTG |
| | | GACAACGAGAAGTCGAACGGCACCATCATCCACGTCAAGGGCAAGCACCTCTGCCCCTC |
| | | GCCCCTGTTCCCCGGCCCCTCCAAGCCCTTCTGGGTGCTGGTCGTGGTTGGTGGCGTCCTC |
| | | GCCTGCTACTCCCTCCTCGTCACGGTGGCGTTCATCATCTTCTGGGTGCGGAGCAAGCGA |
| | | AGCCGCCTCCTGCACAGCGACTACATGAACATGACGCCCCGCCGCCCCGGCCCGACCCG |
| | | GAAGCACTACCAGCCCTACGCCCCGCCGCGCGACTTCGCGGCCTACCGCTCCCGGGTCAA |
| | | GTTCTCCCGCAGCGCCGACGCCCCCGCGTACCAGCAGGGCCAGAACCAGCTCTACAACG |
| | | AGCTCAACCTGGGGAGGCGCGAGGAGTACGACGTGCTTGACAAGCGCCGTGGCCGGGAC |
| | | CCTGAGATGGGGGGTAAGCCGCGACGGAAGAACCCGCAGGAGGGCCTGTACAACGAGC |
| | | TGCAGAAGGACAAGATGGCGGAGGCCTACAGCGAGATCGGGATGAAGGGGGAGCGGCG |
| | | GCGTGGCAAGGGGCACGACGGCCTGTACCAGGGCCTCAGTACGGCCACCAAGGACACCT |
| | | ACGACGCCTTGCACATGCAGGCCCTCCCCCCCCGCTAGTGATGA |
| 442 | Anti-CD19 CAR #5 nucleic | ATGGAGACCGACACCCTCCTGCTGTGGGTGCTGCTGCTTTGGGTGCCCGGGAGCACGGG |
| | acid sequence | GGAGGTGAAGCTGCAGGAGAGCGGCCCCGGCCTGGTGGCCCCCAGCCAGAGCCTGAGCG |
| | | TGACCTGCACCGTGAGCGGCGTGAGCCTGCCCGACTACGGCGTGAGCTGGATCAGGCAG |
| | | CCCCCCAGGAAGGGCCTGGAGTGGCTGGGCGTGATCTGGGGCAGCGAGACCACCTACTA |
| | | CAACAGCGCCCTGAAGAGCAGGCTGACCATCATCAAGGACAACAGCAAGAGCCAGGTGT |
| | | TCCTGAAGATGAACAGCCTGCAGACCGACGACACCGCCATCTACTACTGCGCCAAGCAC |
| | | TACTACTACGGCGGCAGCTACGCCATGGACTACTGGGGCCAGGGCACCAGCGTGACCGT |
| | | GAGCAGCGGCGGCGGCGGCAGCGGCGGCGGCGGCAGCGGCGGCGGCGGCAGCGACATC |
| | | CAGATGACCCAGACCACCAGCAGCCTGAGCGCCAGCCTGGGCGACAGGGTGACCATCAG |
| | | CTGCAGGGCCAGCCAGGACATCAGCAAGTACCTGAACTGGTACCAGCAGAAGCCCGACG |
| | | GCACCGTGAAGCTGCTGATCTACCACACCAGCAGGCTGCACAGCGGCGTGCCCAGCAGG |
| | | TTCAGCGGCAGCGGCAGCGGCACCGACTACAGCCTGACCATCAGCAACCTGGAGCAGGA |
| | | GGACATCGCCACCTACTTCTGCCAGCAGGGCAACACCCTGCCCTACACCTTCGGCGGCGG |
| | | CACCAAGCTGGAGATCAAGGCGAGCACCACCACGCCCGCGCCGCGGCCGCCGACGCCGG |
| | | CGCCCACAATCGCCTCACAGCCACTGAGCCTGCGCCCGGAGGCCTGCAGGCCCGCCGCA |
| | | GGCGGCGCCGTGCACACGCGTGGGCTCGACTTCGCCTGTGACATATACATCTGGGCGCCC |
| | | CTGGCCGGCACCTGCGGCGTCCTGCTGCTGAGCCTGGTGATCACCCTGTATTGCAACCAC |
| | | CGGAACAAGCGCGGCAGGAAGAAGCTCCTGTACATCTTCAAGCAGCCGTTCATGCGCCC |
| | | GGTGCAGACGACCCAGGAGGAGGACGGGTGCAGCTGCCGGTTCCCGGAGGAGGAGGAG |
| | | GGCGGCTGCGAGCTGAGGGTTAAGTTCTCACGGTCGGCCGACGCGCCCGCCTACCAGCA |
| | | GGGCCAGAACCAGCTGTACAATGAGCTGAACCTGGGCCGGAGGGAGGAGTACGACGTA |
| | | CTGGATAAGAGGCGCGGCAGAGACCCCGAGATGGGCGGCAAACCCAGGCGTAAGAACC |
| | | CGCAGGAGGGCCTGTACAACGAACTGCAGAAGGACAAGATGGCCGAGGCCTACAGCGA |
| | | AATAGGCATGAAGGGCGAGCGTAGGCGCGGGAAGGGCCACGACGGCCTGTATCAAGGC |
| | | CTGTCGACCGCCACCAAAGACACCTACGACGCCCTGCACATGCAGGCCCTGCCGCCCAG |
| | | GTAGTGATGA |
| 443 | Anti-CD19 CAR #6 nucleic | ATGGAGACCGACACCCTCCTGCTGTGGGTGCTGCTGCITIGGGTGCCCGGGAGCACGGGC |
| | acid sequence | CAGGTGCAGCTGGTGGAGAGCGGCGGCGGAGTGGTCCAGCCCGGACGCAGCCTGCGACT |
| | | GAGCTGCGCGGCAAGCGGGTTCACCTTCAGCAGCTACGGGATGCACTGGGTCCGTCAGG |
| | | CCCCCGGCAAGGGCCTCGAGTGGGTGGCCGTCATCTGGTACGACGGGAGCAACAAGTAC |
| | | TACGTGGACAGCGTGAAGGGCCGGTTCACGATCAGCAGGGACAACTCCAAGAACACGAT |
| | | GTACCTGCAGATGAACTCCCTGCGGGCCGAGGACACCGCCGTGTATTACTGCGCCAGGC |
| | | GAGGCTACAACTGGAACATCGCCTTCGACATCTGGGGCCAGGGCACCATGGTGACAGTG |
| | | TCGAGCGGAGGCGGCGGCTCCGGAGGCGGAGGCAGCGGCGGCGGCGGATCGGGAGGAG |
| | | GCGGCTCCGACATCCAGATGACCCAGAGCCCAAGCACCCTAAGCGCCAGCGTGGGCGAC |
| | | CGGGTGACCATCACCTGCAGGGCCTCCCAGAGCATCAGCTCCTGGCTGGCCTGGTACCAG |
| | | CAGAAGCCGGGCAAGGCGCCCAAGCTGCTCATCTACAAGGCCTCGAGCCTGGAGAACGG |
| | | CGTGCCCTCCCGCTTCAGCGGCTCCGGCAGCGGAACCGAGTTCACCCTCACAATCAGCTC |
| | | CCTGCAACCCGACGACTTCGCGACCTACTACTGCCAACAGTACAACAGCTACTGGACCTT |
| | | CGGCCAGGGCACCAAGGTCGAGATCAAGGCAAGCAGCCTGCGCCCGGAGGCCTGCAGG |
| | | CCCGCCGCAGGCGGCGCCGTGCACACGCGTGGGCTCGACTTCGCCTGTGACATATACATC |
| | | TGGGCGCCCCTGGCCGGCACCTGCGGCGTCCTGCTGCTGAGCCTGGTGATCACCCTGTAT |
| | | TGCAACCACCGGAACGTGCGCTCCAAACGCAGCCGGCTGCTGCACAGCGACTACATGAA |
| | | CATGACGCCGCGCAGGCCCGGCCCGACCCGAAAGCACTACCAGCCATACGCGCCGCCGC |
| | | GGGACTTCGCCGCCTACCGGTCAAGGGTTAAGTTCTCACGGTCGGCCGACGCGCCCGCCT |
| | | ACCAGCAGGGCCAGAACCAGCTGTACAATGAGCTGAACCTGGGCCGGAGGGAGGAGTA |
| | | CGACGTACTGGATAAGAGGCGCGGCAGAGACCCCGAGATGGGCGGCAAACCCAGGCGT |
| | | AAGAACCCGCAGGAGGGCCTGTACAACGAACTGCAGAAGGACAAGATGGCCGAGGCCT |
| | | ACAGCGAAATAGGCATGAAGGGCGAGCGTAGGCGCGGGAAGGGCCACGACGGCCTGTA |
| | | TCAAGGCCTGTCGACCGCCACCAAAGACACCTACGACGCCCTGCACATGCAGGCCCTGC |
| | | CGCCCAGGTAGTGATGA |
| 444 | Anti-CD19 CAR #7 nucleic | ATGGAGACCGACACCCTCCTGCTGTGGGTGCTGCTGCTTTGGGTGCCCGGGAGCACGGGC |
| | acid sequence | CAGGTCCAGCTCGTGCAGAGCGGGGCGGAGGTGAAGAAGCCCGGGGCATCGGTGAAGG |
| | | TGAGCTGTAAGGCCAGCGGCTACACGTTCACCAGCTACGGCATCAGCTGGGTGCGCCAG |
| | | GCCCCCGGCCAGGGCCTAGAGTGGATGGGGTGGATCAGCGCATACAACGGCAACACCAA |
| | | TTACGCCCAGAAACTGCAGGACCGCGTGACCATGACCACCCACACCTCCACCAGCGCCG |
| | | CATACATGGAGCTGAGGAGCCTGAGGTCCGACGACACCGCCGTGTACTACTGTGCCCGA |
| | | GACGGCGGCCTGGTCGCCGCCTCCCCGGACTACTGGGGCCAGGGGACCCTGGTGACAGT |
| | | GTCCAGTGGAGGCGGCGGCTCCGGAGGCGGAGGCAGCGGCGGCGGCGGATCGGGAGGA |
| | | GGCGGCTCCGACATCGTGATGACACAGAGCCCCGACAGCCTGGCGGTCAGCCTGGGAGA |
| | | GAGGGCCACAATCAACTGCAAGAGCAGCCAGAGTGTCCTCTACAACAGCAACAACAAGA |
| | | ACTATCTCGCCTGGTACCAGCAGAAGCCCGGACAGCCCCCCAAGCTGCTGATCTACCGTG |
| | | CCAGCACCCGGGACAGCGGCGTGCCCGACCGGTTCAGCGGCAGTGGCAGCGGCACGGAC |
| | | TTCACCCTGACCATCTCGAGCCTCCAAGCCGAGGACGTGGCCCTGTACTACTGCCAGCAA |
| | | TACTACTCCACCCCCTACACGTTTGGCCAGGGCACCAAGCTGGAGATCAAAGCGAGCAC |
| | | CACCACGCCCGCGCCGCGGCCGCCGACGCCGGCGCCCACAATCGCCTCACAGCCACTGA |
| | | GCCTGCGCCCGGAGGCCTGCAGGCCCGCCGCAGGCGGCGCCGTGCACACGCGTGGGCTC |
| | | GACTTCGCCTGTGACATATACATCTGGGCGCCCCTGGCCGGCACCTGCGGCGTCCTGCTG |
| | | CTGAGCCTGGTGATCACCCTGTATTGCAACCACCGGAACGTGCGCTCCAAACGCAGCCG |
| | | GCTGCTGCACAGCGACTACATGAACATGACGCCGCGCAGGCCCGGCCCGACCCGAAAGC |
| | | ACTACCAGCCATACGCGCCGCCGCGGGACTTCGCCGCCTACCGGTCAAGGGTTAAGTTCT |
| | | CACGGTCGGCCGACGCGCCCGCCTACCAGCAGGGCCAGAACCAGCTGTACAATGAGCTG |
| | | AACCTGGGCCGGAGGGAGGAGTACGACGTACTGGATAAGAGGCGCGGCAGAGACCCCG |
| | | AGATGGGCGGCAAACCCAGGCGTAAGAACCCGCAGGAGGGCCTGTACAACGAACTGCA |
| | | GAAGGACAAGATGGCCGAGGCCTACAGCGAAATAGGCATGAAGGGCGAGCGTAGGCGC |
| | | GGGAAGGGCCACGACGGCCTGTATCAAGGCCTGTCGACCGCCACCAAAGACACCTACGA |
| | | CGCCCTGCACATGCAGGCCCTGCCGCCCAGGTAGTGATGA |
| 445 | Anti-CD19 CAR #8 nucleic | ATGGAGACCGACACCCTCCTGCTGTGGGTGCTGCTGCTTTGGGTGCCCGGGAGCACGGGC |
| | acid sequence | GAGGTGCAGCTGGTCGAGTCCGGCGGCGGCCTGGTGCAGGCCGGCGAGAGCCTGCGGCT |
| | | CTCGTGCATCGCCAGCGGCCGCACGATGAGTAGCTACGCCATGGCCTGGTTCAGACAGG |
| | | CGCCCGGCAAGGAGAGAGAGTTCGTGGCGGTGAGCTCCCTGTCCGGCGGGACCAGCGAC |
| | | TACGCCGACAGCGTGAAGGGGCGTTTCATCATCAGCAGGGACAACGCCAGGAGCACCGT |
| | | GACCCTCCAGATGAACAACCTGAAGCCCGAGGACACCGCCGTGTACTACTGCGCAGCCC |
| | | GCCCGGTCTACATCCGATACTCCATCCGGGACTATGACATCTGGGGACAGGGCACCCTGG |
| | | TCACCGTGAGCAGCGCCAGCCTTAGCAACAGCATCATGTACTTCAGCCACTTTGTCCCGG |
| | | TCTTCCTGCCAGCCAAGCCGACCACCACGCCCGCGCCGCGGCCGCCGACGCCGGCGCCC |
| | | ACAATCGCCTCACAGCCACTGAGCCTGCGCCCGGAGGCCTGCAGGCCCGCCGCAGGCGG |
| | | CGCCGTGCACACGCGTGGGCTCGACTTCGCCTGTGACATATACATCTGGGCGCCCCTGGC |
| | | CGGCACCTGCGGCGTCCTGCTGCTGAGCCTGGTGATCACCCTGTATTGCAACCACCGGAA |
| | | CGTGCGCTCCAAACGCAGCCGGCTGCTGCACAGCGACTACATGAACATGACGCCGCGCA |
| | | GGCCCGGCCCGACCCGAAAGCACTACCAGCCATACGCGCCGCCGCGGGACTTCGCCGCC |
| | | TACCGGTCAAGGGTTAAGTTCTCACGGTCGGCCGACGCGCCCGCCTACCAGCAGGGCCA |
| | | GAACCAGCTGTACAATGAGCTGAACCTGGGCCGGAGGGAGGAGTACGACGTACTGGATA |
| | | AGAGGCGCGGCAGAGACCCCGAGATGGGCGGCAAACCCAGGCGTAAGAACCCGCAGGA |
| | | GGGCCTGTACAACGAACTGCAGAAGGACAAGATGGCCGAGGCCTACAGCGAAATAGGC |
| | | ATGAAGGGCGAGCGTAGGCGCGGGAAGGGCCACGACGGCCTGTATCAAGGCCTGTCGAC |
| | | CGCCACCAAAGACACCTACGACGCCCTGCACATGCAGGCCCTGCCGCCCAGGTAGTGAT |
| | | GA |
| 446 | Anti-CD19 CAR #9 nucleic | ATGGAGACCGACACCCTCCTGCTGTGGGTGCTGCTGCTTTGGGTGCCCGGGAGCACGGGC |
| | acid sequence | GAGGTGCAACTGGTCGAGAGCGGCGGCGGACTGGTGCAGGCAGGCGAGAGCCTGAGGC |
| | | TGTCCTGCATCGCCAGCGGCCGAACCATGAGCAGCTACGCCATGGCGTGGTTCAGACAG |
| | | GCACCGGGCAAGGAGCGCGAGTTCGTGGCCGTGAGCTCCCTGAGCGGCGGGACCAGCGA |
| | | CTACGCCGACAGCGTGAAGGGCCGGTTCATCATCAGCCGCGACAACGCCAGGTCCACCG |
| | | TGACCCTCCAGATGAACAACCTGAAGCCCGAGGACACGGCAGTGTACTACTGCGCGGCC |
| | | AGGCCCGTCTACATCCGGTACTCGATCCGTGACTACGACATCTGGGGCCAGGGCACCCTC |
| | | GTCACCGTGTCCAGCGCGAGCACCACCACGCCCGCGCCGCGGCCGCCGACGCCGGCGCC |
| | | CACAATCGCCTCACAGCCACTGAGCCTGCGCCCGGAGGCCTGCAGGCCCGCCGCAGGCG |
| | | GCGCCGTGCACACGCGTGGGCTCGACTTCGCCTGTGACATATACATCTGGGCGCCCCTGG |
| | | CCGGCACCTGCGGCGTCCTGCTGCTGAGCCTGGTGATCACCCTGTATTGCAACCACCGGA |
| | | ACGTGCGCTCCAAACGCAGCCGGCTGCTGCACAGCGACTACATGAACATGACGCCGCGC |
| | | AGGCCCGGCCCGACCCGAAAGCACTACCAGCCATACGCGCCGCCGCGGGACTTCGCCGC |
| | | CTACCGGTCAAGGGTTAAGTTCTCACGGTCGGCCGACGCGCCCGCCTACCAGCAGGGCC |
| | | AGAACCAGCTGTACAATGAGCTGAACCTGGGCCGGAGGGAGGAGTACGACGTACTGGAT |
| | | AAGAGGCGCGGCAGAGACCCCGAGATGGGCGGCAAACCCAGGCGTAAGAACCCGCAGG |
| | | AGGGCCTGTACAACGAACTGCAGAAGGACAAGATGGCCGAGGCCTACAGCGAAATAGG |
| | | CATGAAGGGCGAGCGTAGGCGCGGGAAGGGCCACGACGGCCTGTATCAAGGCCTGTCGA |
| | | CCGCCACCAAAGACACCTACGACGCCCTGCACATGCAGGCCCTGCCGCCCAGGTAGTGA |
| | | TGA |
| 447 | Anti-CD19 CAR #10 nucleic | ATGGAGACCGACACCCTCCTGCTGTGGGTGCTGCTGCTTTGGGTGCCCGGGAGCACGGGC |
| | acid sequence | GAGGTGCAGCTGGTCGAGAGCGGCGGAGGCCTGGTGCAGGCCGGCGGCAGCCTGCGTCT |
| | | GAGCTGCGCCTTCAGCGGGCGGACCTTCTCGTCCTACGCCATGGGCTGGTTCCGCCAGGC |
| | | GCCGGGCAAGGAGCGAGAGTTTGTCGCAACGATAAGCCTCTCCGGCGGCACCACGTGGT |
| | | ACGCCGACAGCGTGAAGGGCCGGTTTACCATCAGCAGGGACAACGGAGCGTCCACCGCC |
| | | TACCTGCAGATGAACAGCCTCAAGCCCGAGGATACCGCAGTATACTACTGCGCGGCAAG |
| | | GCCCGTCTACCACAAGACCAGCGCCCGCGACTACGACTTCTGGGGCCAGGGCACCCAGG |
| | | TGACCGTGAGCAGTGCCAGCCTTAGCAACAGCATCATGTACTTCAGCCACTTTGTCCCGG |
| | | TCTTCCTGCCAGCCAAGCCGACCACCACGCCCGCGCCGCGGCCGCCGACGCCGGCGCCC |
| | | ACAATCGCCTCACAGCCACTGAGCCTGCGCCCGGAGGCCTGCAGGCCCGCCGCAGGCGG |
| | | CGCCGTGCACACGCGTGGGCTCGACTTCGCCTGTGACATATACATCTGGGCGCCCCTGGC |
| | | CGGCACCTGCGGCGTCCTGCTGCTGAGCCTGGTGATCACCCTGTATTGCAACCACCGGAA |
| | | CGTGCGCTCCAAACGCAGCCGGCTGCTGCACAGCGACTACATGAACATGACGCCGCGCA |
| | | GGCCCGGCCCGACCCGAAAGCACTACCAGCCATACGCGCCGCCGCGGGACTTCGCCGCC |
| | | TACCGGTCAAGGGTTAAGTTCTCACGGTCGGCCGACGCGCCCGCCTACCAGCAGGGCCA |
| | | GAACCAGCTGTACAATGAGCTGAACCTGGGCCGGAGGGAGGAGTACGACGTACTGGATA |
| | | AGAGGCGCGGCAGAGACCCCGAGATGGGCGGCAAACCCAGGCGTAAGAACCCGCAGGA |
| | | GGGCCTGTACAACGAACTGCAGAAGGACAAGATGGCCGAGGCCTACAGCGAAATAGGC |
| | | ATGAAGGGCGAGCGTAGGCGCGGGAAGGGCCACGACGGCCTGTATCAAGGCCTGTCGAC |
| | | CGCCACCAAAGACACCTACGACGCCCTGCACATGCAGGCCCTGCCGCCCAGGTAGTGAT |
| | | GA |
| 448 | Anti-CD19 CAR #11 nucleic | ATGGAGACCGACACCCTCCTGCTGTGGGTGCTGCTGCTTTGGGTGCCCGGGAGCACGGGC |
| | acid sequence | GAGGTCCAGCTCGTCGAGAGCGGCGGCGGCCTGGTGCAGGCGGGCGGCAGCCTGAGGCT |
| | | GAGTTGCGCGTTCTCCGGCCGGACCTTTAGCAGCTACGCCATGGGCTGGTTCAGGCAGGC |
| | | CCCGGGCAAGGAACGCGAGTTCGTGGCCACCATCTCACTGAGCGGCGGGACGACCTGGT |
| | | ACGCCGACTCCGTGAAGGGCCGCTTCACCATCAGCAGAGACAACGGCGCCAGCACCGCA |
| | | TACCTGCAGATGAACAGCCTCAAGCCCGAGGACACAGCCGTGTACTACTGCGCCGCACG |
| | | GCCCGTGTACCACAAGACCTCCGCCCGAGACTACGACTTCTGGGGACAGGGGACCCAGG |
| | | TCACGGTGAGTTCGGCGAGCACCACCACGCCCGCGCCGCGGCCGCCGACGCCGGCGCCC |
| | | ACAATCGCCTCACAGCCACTGAGCCTGCGCCCGGAGGCCTGCAGGCCCGCCGCAGGCGG |
| | | CGCCGTGCACACGCGTGGGCTCGACTTCGCCTGTGACATATACATCTGGGCGCCCCTGGC |
| | | CGGCACCTGCGGCGTCCTGCTGCTGAGCCTGGTGATCACCCTGTATTGCAACCACCGGAA |
| | | CGTGCGCTCCAAACGCAGCCGGCTGCTGCACAGCGACTACATGAACATGACGCCGCGCA |
| | | GGCCCGGCCCGACCCGAAAGCACTACCAGCCATACGCGCCGCCGCGGGACTTCGCCGCC |
| | | TACCGGTCAAGGGTTAAGTTCTCACGGTCGGCCGACGCGCCCGCCTACCAGCAGGGCCA |
| | | GAACCAGCTGTACAATGAGCTGAACCTGGGCCGGAGGGAGGAGTACGACGTACTGGATA |
| | | AGAGGCGCGGCAGAGACCCCGAGATGGGCGGCAAACCCAGGCGTAAGAACCCGCAGGA |
| | | GGGCCTGTACAACGAACTGCAGAAGGACAAGATGGCCGAGGCCTACAGCGAAATAGGC |
| | | ATGAAGGGCGAGCGTAGGCGCGGGAAGGGCCACGACGGCCTGTATCAAGGCCTGTCGAC |
| | | CGCCACCAAAGACACCTACGACGCCCTGCACATGCAGGCCCTGCCGCCCAGGTAGTGAT |
| | | GA |
| 449 | Anti-CD19 CAR #12 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGAGCGGCGGCGGCCTGGTGCAGGCCGGCGGCAGCCTGCGTCT |
| | | CAGCTGCGCGGCCTCGGGGTTCACCTTCAGCAGCTACGCCATGGCCTGGTTCCGGCAGGC |
| | | CCCGGGCAAGGAGCGAGAGTTTGTGGCGGTCATCAGCCTGAGCGGCGGCTCCGTCGATT |
| | | ACCGGGACAGCGTCAAGGGCCGCTTCACGATCTCCAGGGACAACGCACAGAGCACCGTG |
| | | TCCCTCCAGATGAACAACCTGAAGCCCGAGGACACCGCCATATATTACTGCGCCGCCCGC |
| | | CCGGTGTACATGAAGTGGAGTGAGAGGGACTTCGACTACTGGGGCCAGGGCACACTGGT |
| | | GACCGTGAGCTCCGCCAGCCTTAGCAACAGCATCATGTACTTCAGCCACTTTGTCCCGGT |
| | | CTTCCTGCCAGCCAAGCCGACCACCACGCCCGCGCCGCGGCCGCCGACGCCGGCGCCCA |
| | | CAATCGCCTCACAGCCACTGAGCCTGCGCCCGGAGGCCTGCAGGCCCGCCGCAGGCGGC |
| | | GCCGTGCACACGCGTGGGCTCGACTTCGCCTGTGACATATACATCTGGGCGCCCCTGGCC |
| | | GGCACCTGCGGCGTCCTGCTGCTGAGCCTGGTGATCACCCTGTATTGCAACCACCGGAAC |
| | | GTGCGCTCCAAACGCAGCCGGCTGCTGCACAGCGACTACATGAACATGACGCCGCGCAG |
| | | GCCCGGCCCGACCCGAAAGCACTACCAGCCATACGCGCCGCCGCGGGACTTCGCCGCCT |
| | | ACCGGTCAAGGGTTAAGTTCTCACGGTCGGCCGACGCGCCCGCCTACCAGCAGGGCCAG |
| | | AACCAGCTGTACAATGAGCTGAACCTGGGCCGGAGGGAGGAGTACGACGTACTGGATAA |
| | | GAGGCGCGGCAGAGACCCCGAGATGGGCGGCAAACCCAGGCGTAAGAACCCGCAGGAG |
| | | GGCCTGTACAACGAACTGCAGAAGGACAAGATGGCCGAGGCCTACAGCGAAATAGGCA |
| | | TGAAGGGCGAGCGTAGGCGCGGGAAGGGCCACGACGGCCTGTATCAAGGCCTGTCGACC |
| | | GCCACCAAAGACACCTACGACGCCCTGCACATGCAGGCCCTGCCGCCCAGGTAGTGATG |
| | | A |
| 450 | Anti-CD19 CAR #13 nucleic | ATGGAGACCGACACCCTCCTGCTGTGGGTGCTGCTGCTTTGGGTGCCCGGGAGCACGGGC |
| | acid sequence | GAGGTCCAGCTGGTGGAGTCCGGCGGCGGGCTCGTGCAGGCCGGCGGCAGCCTGAGGCT |
| | | GAGTTGCGCCGCGAGCGGGTTCACCTTCAGCAGCTACGCCATGGCCTGGTTCAGGCAGG |
| | | CGCCGGGCAAGGAGCGCGAGTTCGTGGCCGTCATCAGCCTGTCCGGCGGCAGCGTCGAT |
| | | TACCGGGACAGCGTAAAGGGCCGCTTCACAATCTCCCGAGACAACGCCCAGAGCACAGT |
| | | GTCCCTGCAGATGAACAACCTCAAGCCCGAGGACACGGCCATCTACTACTGCGCCGCCC |
| | | GGCCCGTGTACATGAAGTGGAGTGAGCGTGACTTCGACTACTGGGGGCAGGGCACTCTG |
| | | GTGACCGTGAGCTCGGCGAGCACCACCACGCCCGCGCCGCGGCCGCCGACGCCGGCGCC |
| | | CACAATCGCCTCACAGCCACTGAGCCTGCGCCCGGAGGCCTGCAGGCCCGCCGCAGGCG |
| | | GCGCCGTGCACACGCGTGGGCTCGACTTCGCCTGTGACATATACATCTGGGCGCCCCTGG |
| | | CCGGCACCTGCGGCGTCCTGCTGCTGAGCCTGGTGATCACCCTGTATTGCAACCACCGGA |
| | | ACGTGCGCTCCAAACGCAGCCGGCTGCTGCACAGCGACTACATGAACATGACGCCGCGC |
| | | AGGCCCGGCCCGACCCGAAAGCACTACCAGCCATACGCGCCGCCGCGGGACTTCGCCGC |
| | | CTACCGGTCAAGGGTTAAGTTCTCACGGTCGGCCGACGCGCCCGCCTACCAGCAGGGCC |
| | | AGAACCAGCTGTACAATGAGCTGAACCTGGGCCGGAGGGAGGAGTACGACGTACTGGAT |
| | | AAGAGGCGCGGCAGAGACCCCGAGATGGGCGGCAAACCCAGGCGTAAGAACCCGCAGG |
| | | AGGGCCTGTACAACGAACTGCAGAAGGACAAGATGGCCGAGGCCTACAGCGAAATAGG |
| | | CATGAAGGGCGAGCGTAGGCGCGGGAAGGGCCACGACGGCCTGTATCAAGGCCTGTCGA |
| | | CCGCCACCAAAGACACCTACGACGCCCTGCACATGCAGGCCCTGCCGCCCAGGTAGTGA |
| | | TGA |
| 451 | Anti-CD19 CAR #14 nucleic | ATGGAGACCGACACCCTCCTGCTGTGGGTGCTGCTGCTTTGGGTGCCCGGGAGCACGGGC |
| | acid sequence | GAGGTCCAGCTGGTGGAGTCCGGCGGCGGCCTGGTGCAGGCCGGAGGGAGCCTGAAGCT |
| | | GGCGTGCGCCGCCTCCGTGCACACCAGCTACACGTACGGCATGGGCTGGTTCAGGCAGG |
| | | CGCCCGGCAAGGAGCGCGAGTTCGTCGCCGTGATCAGCTCGGACGGCGTCCGAACCAAC |
| | | TACGCGGACAGCGTGCAGGGCCGGTTCACCATCAGCAGGGACAACGCCAAGAACACCAT |
| | | CTACCTCCAGATGAACAACCTGAAGACCGAGGACAGCGCCATCTACTACTGCGCCGCCG |
| | | GGAGCGGCCTCTTCTCCGCAGGCTACGAGTATCAGTATTGGGGCCAGGGCACCCAGGTG |
| | | ACCGTGAGCAGCGCGGCCGCGATAGAGGTAATGTACCCTCCTCCTTACCTGGACAACGA |
| | | GAAGTCGAACGGCACCATCATCCACGTCAAGGGCAAGCACCTCTGCCCGTCGCCGCTGTT |
| | | CCCGGGCCCGTCCAAGCCCTTCTGGGTGCTGGTCGTGGTTGGTGGCGTCCTCGCCTGCTA |
| | | CTCCCTCCTCGTCACGGTGGCGTTCATCATCTTCTGGGTGCGGAGCAAGCGAAGCCGCCT |
| | | CCTGCACAGCGACTACATGAACATGACGCCGCGCCGCCCGGGCCCGACCCGGAAGCACT |
| | | ACCAGCCCTACGCGCCGCCGCGCGACTTCGCGGCCTACCGCTCCCGGGTCAAGTTCTCCC |
| | | GCAGCGCCGACGCGCCCGCGTACCAGCAGGGCCAGAACCAGCTCTACAACGAGCTCAAC |
| | | CTGGGGAGGCGCGAGGAGTACGACGTGCTTGACAAGCGCCGTGGCCGGGACCCTGAGAT |
| | | GGGCGGTAAGCCGCGACGGAAGAACCCGCAGGAGGGCCTGTACAACGAGCTGCAGAAG |
| | | GACAAGATGGCGGAGGCCTACAGCGAGATCGGGATGAAGGGCGAGCGGCGGCGTGGCA |
| | | AGGGCCACGACGGCCTGTACCAGGGCCTCAGTACGGCCACCAAGGACACCTACGACGCC |
| | | TTGCACATGCAGGCCCTCCCGCCGCGCTAGTGATGA |
| 452 | Anti-CD19 CAR #15 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGTCCGGCGGCGGCCTGGTCCAGGCCGGCGGCTCACTGAAGCT |
| | | GGCCTGCGCGGCGAGCGTCCACACCTCGTACACCTACGGCATGGGCTGGTTCAGGCAGG |
| | | CGCCCGGGAAGGAGCGGGAGTTCGTGGCCGTGATCAGTAGCGACGGCGTGCGCACCAAC |
| | | TACGCCGACAGCGTGCAGGGCCGATTCACCATAAGCAGGGACAACGCCAAGAACACCAT |
| | | CTACCTCCAGATGAACAACCTCAAGACGGAGGACAGCGCCATCTACTATTGCGCCGCCG |
| | | GCTCCGGGCTGTTCTCCGCCGGCTACGAGTACCAGTACTGGGGCCAGGGCACCCAGGTC |
| | | ACCGTGAGCAGCGCGAGCACCACCACGCCCGCGCCGCGGCCGCCGACGCCGGCGCCCAC |
| | | AATCGCCTCACAGCCACTGAGCCTGCGCCCGGAGGCCTGCAGGCCCGCCGCAGGCGGCG |
| | | CCGTGCACACGCGTGGGCTCGACTTCGCCTGTGACATATACATCTGGGCGCCCCTGGCCG |
| | | GCACCTGCGGCGTCCTGCTGCTGAGCCTGGTGATCACCCTGTATTGCAACCACCGGAACG |
| | | TGCGCTCCAAACGCAGCCGGCTGCTGCACAGCGACTACATGAACATGACGCCGCGCAGG |
| | | CCCGGCCCGACCCGAAAGCACTACCAGCCATACGCGCCGCCGCGGGACTTCGCCGCCTA |
| | | CCGGTCAAGGGTTAAGTTCTCACGGTCGGCCGACGCGCCCGCCTACCAGCAGGGCCAGA |
| | | ACCAGCTGTACAATGAGCTGAACCTGGGCCGGAGGGAGGAGTACGACGTACTGGATAAG |
| | | AGGCGCGGCAGAGACCCCGAGATGGGCGGCAAACCCAGGCGTAAGAACCCGCAGGAGG |
| | | GCCTGTACAACGAACTGCAGAAGGACAAGATGGCCGAGGCCTACAGCGAAATAGGCAT |
| | | GAAGGGCGAGCGTAGGCGCGGGAAGGGCCACGACGGCCTGTATCAAGGCCTGTCGACC |
| | | GCCACCAAAGACACCTACGACGCCCTGCACATGCAGGCCCTGCCGCCCAGGTAGTGATG |
| | | A |
| 453 | Anti-CD19 CAR #16 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGAGCGGAGGCGGCCTGGTGCAGGCGGAGGGCAGCCTCCGGC |
| | | TGAGCTGCACGGCCAGCGGACTGACCTTTGACAACTACGCCATGGGCTGGTTCCGACAG |
| | | GCCCCGGGCAAGGAGAGGGAGTTCGTGGCCACCATCTCCGCCGCGGGCCACAGAACCAT |
| | | CTTCACCAACAGCGTGCGCGGGAGGTTTACCGCCTCGCGGGACAACGCCAAGAAGACCA |
| | | CGTACCTGCAGATGAATAGCCTGAAGCCGGAGGACACCGCCGTCTACTACTGCGCCGCC |
| | | TCCCCAAGCCAGTACGGGAGCATGGGCAAGTCCGCCTACGACTATTGGGGCCAGGGCAC |
| | | CCTCGTGACCGTCAGCAGCGCCAGCCTTAGCAACAGCATCATGTACTTCAGCCACTTTGT |
| | | CCCGGTCTTCCTGCCAGCCAAGCCGACCACCACGCCCGCGCCGCGGCCGCCGACGCCGG |
| | | CGCCCACAATCGCCTCACAGCCACTGAGCCTGCGCCCGGAGGCCTGCAGGCCCGCCGCA |
| | | GGCGGCGCCGTGCACACGCGTGGGCTCGACTTCGCCTGTGACATATACATCTGGGCGCCC |
| | | CTGGCCGGCACCTGCGGCGTCCTGCTGCTGAGCCTGGTGATCACCCTGTATTGCAACCAC |
| | | CGGAACGTGCGCTCCAAACGCAGCCGGCTGCTGCACAGCGACTACATGAACATGACGCC |
| | | GCGCAGGCCCGGCCCGACCCGAAAGCACTACCAGCCATACGCGCCGCCGCGGGACTTCG |
| | | CCGCCTACCGGTCAAGGGTTAAGTTCTCACGGTCGGCCGACGCGCCCGCCTACCAGCAG |
| | | GGCCAGAACCAGCTGTACAATGAGCTGAACCTGGGCCGGAGGGAGGAGTACGACGTACT |
| | | GGATAAGAGGCGCGGCAGAGACCCCGAGATGGGCGGCAAACCCAGGCGTAAGAACCCG |
| | | CAGGAGGGCCTGTACAACGAACTGCAGAAGGACAAGATGGCCGAGGCCTACAGCGAAA |
| | | TAGGCATGAAGGGCGAGCGTAGGCGCGGGAAGGGCCACGACGGCCTGTATCAAGGCCT |
| | | GTCGACCGCCACCAAAGACACCTACGACGCCCTGCACATGCAGGCCCTGCCGCCCAGGT |
| | | AGTGATGA |
| 454 | Anti-CD19 CAR #17 nucleic | ATGGAGACCGACACCCTCCTGCTGTGGGTGCTGCTGCTTTGGGTGCCCGGGAGCACGGGC |
| | acid sequence | GAGGTCCAACTGGTGGAGAGCGGAGGCGGACTCGTGCAGGCCGAGGGCTCCCTGCGGCT |
| | | GAGCTGCACCGCATCCGGCCTCACATTCGACAACTACGCCATGGGCTGGTTCCGCCAGGC |
| | | CCCAGGCAAGGAAAGGGAGTTCGTGGCGACGATCAGCGCGGCCGGCCACCGCACCATCT |
| | | TCACCAACAGCGTGCGCGGAAGGTTCACCGCCAGCCGAGACAACGCCAAGAAGACCACG |
| | | TACCTGCAGATGAACTCCCTGAAGCCCGAGGACACCGCCGTGTACTACTGTGCCGCCAGC |
| | | CCGAGCCAGTACGGGAGCATGGGCAAGAGCGCCTACGACTACTGGGGCCAGGGCACCCT |
| | | GGTCACCGTGTCGAGCGCGAGCACCACCACGCCCGCGCCGCGGCCGCCGACGCCGGCGC |
| | | CCACAATCGCCTCACAGCCACTGAGCCTGCGCCCGGAGGCCTGCAGGCCCGCCGCAGGC |
| | | GGCGCCGTGCACACGCGTGGGCTCGACTTCGCCTGTGACATATACATCTGGGCGCCCCTG |
| | | GCCGGCACCTGCGGCGTCCTGCTGCTGAGCCTGGTGATCACCCTGTATTGCAACCACCGG |
| | | AACGTGCGCTCCAAACGCAGCCGGCTGCTGCACAGCGACTACATGAACATGACGCCGCG |
| | | CAGGCCCGGCCCGACCCGAAAGCACTACCAGCCATACGCGCCGCCGCGGGACTTCGCCG |
| | | CCTACCGGTCAAGGGTTAAGTTCTCACGGTCGGCCGACGCGCCCGCCTACCAGCAGGGC |
| | | CAGAACCAGCTGTACAATGAGCTGAACCTGGGCCGGAGGGAGGAGTACGACGTACTGGA |
| | | TAAGAGGCGCGGCAGAGACCCCGAGATGGGCGGCAAACCCAGGCGTAAGAACCCGCAG |
| | | GAGGGCCTGTACAACGAACTGCAGAAGGACAAGATGGCCGAGGCCTACAGCGAAATAG |
| | | GCATGAAGGGCGAGCGTAGGCGCGGGAAGGGCCACGACGGCCTGTATCAAGGCCTGTCG |
| | | ACCGCCACCAAAGACACCTACGACGCCCTGCACATGCAGGCCCTGCCGCCCAGGTAGTG |
| | | ATGA |
| 435 | Anti-CD19 CAR #18 nucleic | ATGGAGACCGACACCCTCCTGCTGTGGGTGCTGCTGCTTTGGGTGCCCGGGAGCACGGGC |
| | acid sequence | GAGGTCCAACTGGTGGAGAGCGGAGGCGGACTCGTGCAGGCCGAGGGCTCCCTGCGGCT |
| | | GAGCTGCACCGCATCCGGCCTCACATTCGACAACTACGCCATGGGCTGGTTCCGCCAGGC |
| | | CCCAGGCAAGGAAAGGGAGTTCGTGGCGACGATCAGCGCGGCCGGCCACCGCACCATCT |
| | | TCACCAACAGCGTGCGCGGAAGGTTCACCGCCAGCCGAGACAACGCCAAGAAGACCACG |
| | | TACCTGCAGATGAACTCCCTGAAGCCCGAGGACACCGCCGTGTACTACTGTGCCGCCAGC |
| | | CCGAGCCAGTACGGGAGCATGGGCAAGAGCGCCTACGACTACTGGGGCCAGGGCACCCT |
| | | GGTCACCGTGTCGAGCGGAGGCGGCGGCTCCGGAGGCGGAGGCAGCGGCGGCGGCGGA |
| | | TCGGAGGTGCAGCTGGTGGAGTCCGGCGGCGGGCTGGTGCAGGCCGGCGACAGCCTGAG |
| | | GCTCTCGTGCGCCGCCAGCGGCAGGACCCTGAGCAGCTACACGACCGGCTGGTTCCGTC |
| | | AGGGGCTGGGCAAGGAGCGCGAGTTCGTGGCCGCGATCACCAAGAACGGCCCCTACCTC |
| | | TACTACAAGGACAGCGTCAGGGGCCGCTTCACCATCTCCCGAGACAGCGCCAAGACCAC |
| | | CGTGTTCCTGCAGATGGACAACCTGAAGCCGGAGGACACCGCCGTCTATTACTGCGCCGT |
| | | AAGCGACGCCCAGTACGACATCCGGAACCGGGAGAAGTACTACTACTGGGGCCAGGGCA |
| | | CCCTGGTGACGGTCTCCAGCGCGGCCGCGATAGAGGTAATGTACCCTCCTCCTTACCTGG |
| | | ACAACGAGAAGTCGAACGGCACCATCATCCACGTCAAGGGCAAGCACCTCTGCCCGTCG |
| | | CCGCTGTTCCCGGGCCCGTCCAAGCCCTTCTGGGTGCTGGTCGTGGTTGGTGGCGTCCTC |
| | | GCCTGCTACTCCCTCCTCGTCACGGTGGCGTTCATCATCTTCTGGGTGCGGAGCAAGCGA |
| | | AGCCGCCTCCTGCACAGCGACTACATGAACATGACGCCGCGCCGCCCGGGCCCGACCCG |
| | | GAAGCACTACCAGCCCTACGCGCCGCCGCGCGACTTCGCGGCCTACCGCTCCCGGGTCA |
| | | AGTTCTCCCGCAGCGCCGACGCGCCCGCGTACCAGCAGGGCCAGAACCAGCTCTACAAC |
| | | GAGCTCAACCTGGGGAGGCGCGAGGAGTACGACGTGCTTGACAAGCGCCGTGGCCGGGA |
| | | CCCTGAGATGGGCGGTAAGCCGCGACGGAAGAACCCGCAGGAGGGCCTGTACAACGAG |
| | | CTGCAGAAGGACAAGATGGCGGAGGCCTACAGCGAGATCGGGATGAAGGGCGAGCGGC |
| | | GGCGTGGCAAGGGCCACGACGGCCTGTACCAGGGCCTCAGTACGGCCACCAAGGACACC |
| | | TACGACGCCTTGCACATGCAGGCCCTCCCGCCGCGCTAGTGATGA |
| 456 | Anti-CD19 CAR #19 nucleic | ATGGAGACCGACACCCTCCTGCTGTGGGTGCTGCTGCTTTGGGTGCCCGGGAGCACGGGC |
| | acid sequence | GAGGTCCAACTGGTGGAGAGCGGAGGCGGACTCGTGCAGGCCGAGGGCTCCCTGCGGCT |
| | | GAGCTGCACCGCATCCGGCCTCACATTCGACAACTACGCCATGGGCTGGTTCCGCCAGGC |
| | | CCCAGGCAAGGAAAGGGAGTTCGTGGCGACGATCAGCGCGGCCGGCCACCGCACCATCT |
| | | TCACCAACAGCGTGCGCGGAAGGTTCACCGCCAGCCGAGACAACGCCAAGAAGACCACG |
| | | TACCTGCAGATGAACTCCCTGAAGCCCGAGGACACCGCCGTGTACTACTGTGCCGCCAGC |
| | | CCGAGCCAGTACGGGAGCATGGGCAAGAGCGCCTACGACTACTGGGGCCAGGGCACCCT |
| | | GGTCACCGTGTCGAGCGGAGGCGGCGGCTCCGGAGGCGGAGGCAGCGGCGGCGGCGGA |
| | | TCGGAGGTCCAGCTGGTGGAGAGCGGCGGCGGGCTCGTGCAGCCCGGCGGGAGCCTCAG |
| | | TCTGAGCTGCGCCGCCAGCGGAGACACCTTCGGGACCAAGGCCATGGGCTGGTTCAGGC |
| | | AGGCGCCGGGCAAGGGCCGAGAATTCGTGGCCGCCATCACGTGGACCGCCGGCCTGACC |
| | | TACCTGGCGGACTCCGCCAAGGGCCGGTTTACCATCTCCAAGGACCGCGTGCAGAACAC |
| | | CGTCTACCTGGAGATGAACAACCTGGAGCCCGAGGACACAGCCGTCTACTACTGCGCCT |
| | | CGCGGAGGACCTTCAGCTACGAACACCCCAACTACTACACCGCCTCCGCGTACCACTTCT |
| | | GGGGACAGGGCACGCTGGTGACCGTGAGCAGCGCGGCCGCGATAGAGGTAATGTACCCT |
| | | CCTCCTTACCTGGACAACGAGAAGTCGAACGGCACCATCATCCACGTCAAGGGCAAGCA |
| | | CCTCTGCCCGTCGCCGCTGTTCCCGGGCCCGTCCAAGCCCTTCTGGGTGCTGGTCGTGGTT |
| | | GGTGGCGTCCTCGCCTGCTACTCCCTCCTCGTCACGGTGGCGTTCATCATCTTCTGGGTGC |
| | | GGAGCAAGCGAAGCCGCCTCCTGCACAGCGACTACATGAACATGACGCCGCGCCGCCCG |
| | | GGCCCGACCCGGAAGCACTACCAGCCCTACGCGCCGCCGCGCGACTTCGCGGCCTACCG |
| | | CTCCCGGGTCAAGTTCTCCCGCAGCGCCGACGCGCCCGCGTACCAGCAGGGCCAGAACC |
| | | AGCTCTACAACGAGCTCAACCTGGGGAGGCGCGAGGAGTACGACGTGCTTGACAAGCGC |
| | | CGTGGCCGGGACCCTGAGATGGGCGGTAAGCCGCGACGGAAGAACCCGCAGGAGGGCC |
| | | TGTACAACGAGCTGCAGAAGGACAAGATGGCGGAGGCCTACAGCGAGATCGGGATGAA |
| | | GGGCGAGCGGCGGCGTGGCAAGGGCCACGACGGCCTGTACCAGGGCCTCAGTACGGCCA |
| | | CCAAGGACACCTACGACGCCTTGCACATGCAGGCCCTCCCGCCGCGCTAGTGATGA |
| 457 | Anti-CD19 CAR #20 nucleic | ATGGAGACCGACACCCTCCTGCTGTGGGTGCTGCTGCTTTGGGTGCCCGGGAGCACGGGC |
| | acid sequence | GAGGTCCAACTGGTGGAGAGCGGAGGCGGACTCGTGCAGGCCGAGGGCTCCCTGCGGCT |
| | | GAGCTGCACCGCATCCGGCCTCACATTCGACAACTACGCCATGGGCTGGTTCCGCCAGGC |
| | | CCCAGGCAAGGAAAGGGAGTTCGTGGCGACGATCAGCGCGGCCGGCCACCGCACCATCT |
| | | TCACCAACAGCGTGCGCGGAAGGTTCACCGCCAGCCGAGACAACGCCAAGAAGACCACG |
| | | TACCTGCAGATGAACTCCCTGAAGCCCGAGGACACCGCCGTGTACTACTGTGCCGCCAGC |
| | | CCGAGCCAGTACGGGAGCATGGGCAAGAGCGCCTACGACTACTGGGGCCAGGGCACCCT |
| | | GGTCACCGTGTCGAGCGGAGGCGGCGGCTCCGGAGGCGGAGGCAGCGGCGGCGGCGGA |
| | | TCGGAGGTGCAGCTCGTCGAGAGCGGCGGCGGCCTGGTACAGACCGGCGACAGCCTCCG |
| | | TCTGACCTGCGCCGCCAGCGGCCGCACCATCGGCGACCGGTCCATGGCGTGGTTCAGGC |
| | | AGGCCCCGGGCAAGGAGCGGGAGTTCGTCATCGTCAACACGTGGAACGGCTACACCTAC |
| | | ACCGACGAGAGCGTGGAGGGCCGCTTCACCATCAGCAGGGACAGCGGCGCCAACACGGT |
| | | GCACCTGCAGATGAACAAGCTGAACCCCAAGGACACCGCCGTGTACTACTGCGCCTCCT |
| | | CCAGCTACACCTATAACATCAGGCAGCGAAGCTCGTACGACTGGTGGGGCCAGGGGACG |
| | | CTGGTGACCGTGAGCAGCGCGGCCGCGATAGAGGTAATGTACCCTCCTCCTTACCTGGAC |
| | | AACGAGAAGTCGAACGGCACCATCATCCACGTCAAGGGCAAGCACCTCTGCCCGTCGCC |
| | | GCTGTTCCCGGGCCCGTCCAAGCCCTTCTGGGTGCTGGTCGTGGTTGGTGGCGTCCTCGC |
| | | CTGCTACTCCCTCCTCGTCACGGTGGCGTTCATCATCTTCTGGGTGCGGAGCAAGCGAAG |
| | | CCGCCTCCTGCACAGCGACTACATGAACATGACGCCGCGCCGCCCGGGCCCGACCCGGA |
| | | AGCACTACCAGCCCTACGCGCCGCCGCGCGACTTCGCGGCCTACCGCTCCCGGGTCAAGT |
| | | TCTCCCGCAGCGCCGACGCGCCCGCGTACCAGCAGGGCCAGAACCAGCTCTACAACGAG |
| | | CTCAACCTGGGGAGGCGCGAGGAGTACGACGTGCTTGACAAGCGCCGTGGCCGGGACCC |
| | | TGAGATGGGCGGTAAGCCGCGACGGAAGAACCCGCAGGAGGGCCTGTACAACGAGCTG |
| | | CAGAAGGACAAGATGGCGGAGGCCTACAGCGAGATCGGGATGAAGGGCGAGCGGCGGC |
| | | GTGGCAAGGGCCACGACGGCCTGTACCAGGGCCTCAGTACGGCCACCAAGGACACCTAC |
| | | GACGCCTTGCACATGCAGGCCCTCCCGCCGCGCTAGTGATGA |
| 458 | Anti-CD19 CAR #21 nucleic | ATGGAGACCGACACCCTCCTGCTGTGGGTGCTGCTGCTTTGGGTGCCCGGGAGCACGGGC |
| | acid sequence | GAAGTGCAGCTGGTCGAGAGCGGCGGCGGCCTGGTGCAGGCCGGCGGGAGCCTCAGACT |
| | | GAGCTGCGCCGGGTCCGGCTTCACCTTCTCCTCGACCGTGATAGGCTGGTACCGCCAGGC |
| | | GCCGGGCAAGCAGCGGGAATTCGTGGCCCTGATCACCGCCGGCGGCCACACCAACAGCG |
| | | TCGAGGACCGATTCATCATTTCCAGGGACAACGCCAAGAGCACCGCCTACCTGCAGATG |
| | | ACCAGCCTCAAGAGCGAGGACACCGCCGTGTACTACTGCAACCTGCCCAACCAGGGCTA |
| | | CTGGGGCCAGGGGACCCTGGTCACGGTGAGCAGCGCGGCCGCGATAGAGGTAATGTACC |
| | | CTCCTCCTTACCTGGACAACGAGAAGTCGAACGGCACCATCATCCACGTCAAGGGCAAG |
| | | CACCTCTGCCCGTCGCCGCTGTTCCCGGGCCCGTCCAAGCCCTTCTGGGTGCTGGTCGTG |
| | | GTTGGTGGCGTCCTCGCCTGCTACTCCCTCCTCGTCACGGTGGCGTTCATCATCTTCTGGG |
| | | TGCGGAGCAAGCGAAGCCGCCTCCTGCACAGCGACTACATGAACATGACGCCGCGCCGC |
| | | CCGGGCCCGACCCGGAAGCACTACCAGCCCTACGCGCCGCCGCGCGACTTCGCGGCCTA |
| | | CCGCTCCCGGGTCAAGTTCTCCCGCAGCGCCGACGCGCCCGCGTACCAGCAGGGCCAGA |
| | | ACCAGCTCTACAACGAGCTCAACCTGGGGAGGCGCGAGGAGTACGACGTGCTTGACAAG |
| | | CGCCGTGGCCGGGACCCTGAGATGGGCGGTAAGCCGCGACGGAAGAACCCGCAGGAGG |
| | | GCCTGTACAACGAGCTGCAGAAGGACAAGATGGCGGAGGCCTACAGCGAGATCGGGAT |
| | | GAAGGGCGAGCGGCGGCGTGGCAAGGGCCACGACGGCCTGTACCAGGGCCTCAGTACG |
| | | GCCACCAAGGACACCTACGACGCCTTGCACATGCAGGCCCTCCCGCCGCGCTAGTGATG |
| | | A |
| 459 | | GFX1FX2X3YG; X1 is T or I, X2 is S or R, X3 is S or N |
| 460 | | IYYDGX4X5X6; X4 is N or S, X5 is K or N, X6 is K or N |
| 461 | | ARELTGDAFDX7; X7 is I or L |
| 462 | | QX8IGSX9; X8 is S or R, X9 is S or H |
| 463 | | SANDWTVDHPQTLFAWEGACIRIPCKYKTPLPKARLDNILLFQNYEFDKATKKFTGTVLYNA |
| | | TKTEKDPESELYLSKQGRVTFLGNRIDNCTLKIHPIRANDSGNLGLRMTAGTERWMEPIHLNV |
| | | SEKPFQPYIQMPSEIRESQSVTLTCGLNFSCFGYDILLKWFLEDSEITSITSSVTSITSSVTSSIKN |
| | | VYTESKLTFQPKWTDHGKSVKCQVQHSSKVLSERTVRLDVKYTPKLEIKVNPTEVEKNNSVT |
| | | MTCRVNSSNPKLRTVAVSWFKDGRPLEDQELEQEQQMSKLILHSVTKDMRGKYRCQASNDI |
| | | GPGESEEVELTVHYAPEPSRVHIYPSPAEEGQSVELICESLASPSATNYTWYHNRKPIPGDTQE |
| | | KLRIPKVSPWHAGNYSCLAENRLGHGKIDQEAKLDVHYAPKAVTTVIQSFTPILEGDSVTLVC |
| | | RYNSSNPDVTSYRWNPQGSGSVLKPGVLRIQKVTWDSMPVSCAACNHKCSWALPVILNVHY |
| | | APRDVKVLKVSPASEIRAGQRVLLQCDFAESNPAEVRFFWKKNGSLVQEGRYLSFGSVSPED |
| | | SGNYNCMVNNSIGETLSQAWNLQVLYAPRRLRVSISPGDHVMEGKKATLSCESDANPPISQY |
| | | TWFDSSGQDLHSSGQK |
| 464 | | HQSSX10EPYT; X10 is T, R, or S |
| 465 | | CAGGTGCAGCTGGTGGAATCTGGGGGAGACGTGGTCCAGCCTGGGAGGTCCCTGAGACT |
| | | CTCCTGTGCAGCGTCTGGATTCATCTTCAGGAACTATGGCATGCACTGGGTCCGCCAGGC |
| | | TCCAGGCAAGGGGCTGGAGTGGGTGGCAGTCATTTACTATGATGGAAGTAAAAATTACT |
| | | ATGAAGACTCCGTGAAGGGCCGATTCACCATCTCCAGAGACAATTCCAAAAACACGCTG |
| | | TATCTGCAAATGAACAGCCTGAGAGTCGAGGACACGGCTGTGTATTACTGTGCGAGAGA |
| | | ACTTACTGGGGATGCTTTTGATATCTGGGGCCAAGGGACAATGGTCACCGTCTCTTCA |
| 466 | | GAAATTGTGCTGACTCAGTCTCCAGACTTTCAGTCTCTGACTCCAAAGGAGAAAGTCACC |
| | | ATCACCTGTCGGGCCAGTCAGAGAATTGGTAGTAGCTTACACTGGTACCAGCAGAAACC |
| | | AGATCAGTCTCCAAAACTCCTCATCAAGTATGCTTCCCAGTCCTTCTCAGGGGTCCCCTC |
| | | GAGGTTCAGTGGCAGTGGATCTGGGACAGATTTCACCCTCACCATCAATAGCCTGGATGC |
| | | TGAAGATGCTGCAACGTATTACTGTCATCAGAGTAGTAGTTTCCCGTACACTTTTGGCCA |
| | | GGGGACCAAGCTGCAGATCAAA |
| 467 | | CAGGTGCAGCTGGTGGAGTCTGGGGGAGGCGTGGTCCAGCCTGGGAGGTCCCTGAGACT |
| | | CTCCTGTGCAGCGTCTGGATTCACCTTCAGTAGCTATGGCATGCACTGGGTCCGCCAGGC |
| | | TCCAGGCAAGGGGCTGGAGTGGGTGGCGGTTATATACTATGATGGAAATAAAAAATACT |
| | | ATGTTGACTCCGTGAAGGACCGAATCACCATCTCCAGAGACAATTCCAAGAACACGTTGT |
| | | ATCTGCAAATGAACAGCCTGAGAGCCGAGGACACGGCTGTGTATTATTGTGCGAGAGAA |
| | | CTAACTGGGGATGCTTTTGATATCTGGGGCCAAGGGACAATGGTCACCGTCTCTTCA |
| 468 | | GAAATTGTGCTGACTCAGTCTCCAGACTTTCAGTCTGTGACTCCAAAGGAGAAAGTCACC |
| | | ATCACCTGCCGGGCCAGTCAGAGAATTGGAAGTAGCTTACACTGGTACCAGCAGAAATC |
| | | AGATCAGTCTCCAAAGCTCCTCATCAAGTATGCTTCCCAGTCCTTCTCAGGGGTCCCCTC |
| | | GAGGTTCAGTGGCAGTGGATCTGGGACAGATTTCACCCTCACCATCAATAGCCTGGAAG |
| | | CTGAAGATGCTGCAACGTATTACTGTCATCAGAGTAGTACGTTCCCGTACACTTTTGGCC |
| | | AGGGGACCAAGCTAGAGATCAAA |
| 469 | | CAGGTGCAGCTGGTGGAGTCTGGGGGGGGCGTGGTCCAGCCTGGGAGGTCCCTGAGACT |
| | | CTCCTGTGCAGCGTCTGGATTCACCTTCAGTAGCTATGGCATGCACTGGGTCCGCCAGAC |
| | | TCCAGGCAAGGGGCTGGAGTGGGTGGCAGTTATATACTATGATGGAAATAATAAATACT |
| | | ATGCAGACTCCGTGAAGGGCCGATTCACCATCTCCAGAGAGAATTCCAAGAACACGCTG |
| | | TATCTGCAAATGAACAGCCTGAGAGCCGATGACACGGCTCTATATTACTGTGCGAGAGA |
| | | ACTAACTGGGGATGCTTTTGATCTCTGGGGCCAAGGGACAATGGTCACCGTCTCTTCA |
| 470 | | GAAATTGTGCTGACTCAGTCTCCAGATTTTCAGTCTGCGACTCCAAAGGAGAAAGTCACC |
| | | ATCACCTGCCGGGCCAGTCAGAGCATTGGTAGTCACTTACACTGGTATCAGCAGAAACC |
| | | AGATCAGTCTCCAAAGCTCCTCATCAAGTATGCTTCCCAGTCCTTCTCAGGGGTCCCCTC |
| | | GAGGTTCAGTGGCAGTGGATCTGGGACAGATTTCACCCTCACCATCAATAGACTGGAAG |
| | | ATGAAGATGCTGCAACGTATTACTGTCATCAGAGTAGTAGGTTCCCGTACACTTTTGGCC |
| | | AGGGGACCAAGCTGGATATCAAA |
| 471 | | DSSKWVFEHPETLYAWEGACVWIPCTYRALDGDLESFILFHNPEYNKNTSKFDGTRLYESTK |
| | | DGKVPSEQKRVQFLGDKNKNCTLSIHPVHLNDSGQLGLRMESKTEKWMERIHLNVSERPFPP |
| | | HIQLPPEIQESQEVTLTCLLNFSCYGYPIQLQWLLEGVPMRQAAVTSTSLTIKSVFTRSELKFSP |
| | | QWSHHGKIVTCQLQDADGKFLSNDTVQLNVKHTPKLEIKVTPSDAIVREGDSVTMTCEVSSS |
| | | NPEYTTVSWLKDGTSLKKQNTFTLNLREVTKDQSGKYCCQVSNDVGPGRSEEVFLQVQYAP |
| | | EPSTVQILHSPAVEGSQVEFLCMSLANPLPTNYTWYHNGKEMQGRTEEKVHIPKILPWHAGT |
| | | YSCVAENILGTGQRGPGAELDVQYPPKKVTTVIQNPMPIREGDTVTLSCNYNSSNPSVTRYE |
| | | WKPHGAWEEPSLGVLKIQNVGWDNTTIACAACNSWCSWASPVALNVQYAPRDVRVRKIKP |
| | | LSEIHSGNSVSLQCDFSSSHPKEVQFFWEKNGRLLGKESQLNFDSISPEDAGSYSCWVNNSIG |
| | | QTASKAWTLEVLYAPRRLRVSMSPGDQVMEGKSATLTCESDANPPVSHYTWFDWNNQSLP |
| | | YHSQKLRLEPVKVQHSGAYWCQGTNSVGKGRSPLSTLTVYYSPETIGRR |
| 472 | | DSSKWVFEHPETLYAWEGACVWIPCTYRALDGDLESFILFHNPEYNKNTSKFDGTRLYESTK |
| | | DGKVPSEQKRVQFLGDKNKNCTLSIHPVHLNDSGQLGLRMESKTEKWMERIHLNVSERPFPP |
| | | HIQLPPEIQESQEVTLTCLLNFSCYGYPIQLQWLLEGVPMRQAAVTSTSLTIKSVFTRSELKFSP |
| | | QWSHHGKIVTCQLQDADGKFLSNDTVQLNVKHTPKLEIKVTPSDAIVREGDSVTMTCEVSSS |
| | | NPEYTTVSWLKDGTSLKKQNTFTLNLREVTKDQSGKYCCQVSNDVGPGRSEEVFLQVQYAP |
| | | EPSTVQILHSPAVEGSQVEFLCMSLANPLPTNYTWYHNGKEMQGRTEEKVHIPKILPWHAGT |
| | | YSCVAENILGTGQRGPGAELDVQYPPKKVTTVIQNPMPIREGDTVTLSCNYNSSNPSVTRYE |
| | | WKPHGAWEEPSLGVLKIQNVGWDNTTIACAACNSWCSWASPVALNVQYAPRDVRVRKIKP |
| | | LSEIHSGNSVSLQCDFSSSHPKEVQFFWEKNGRLLGKESQLNFDSISPEDAGSYSCWVNNSIG |
| | | QTASKAWTLEVLYAPRRLRVSMSPGDQVMEGKSATLTCESDANPPVSHYTWFDWNNQSLP |
| | | YHSQKLRLEPVKVQHSGAYWCQGTNSVGKGRSPLSTLTVYYSPETIGRRGGGGSGLNDIFEA |
| | | QKIEWHEGGGGSHHHHHH |
| 473 | | QYAPEPSTVQILHSPAVEGSQVEFLCMSLANPLPTNYTWYANGKEMQGRTEEKVHIPKILPW |
| | | HAGTYSCVAENILGTGQRGPGAELDVQYPPKKVTTVIQNPMPIREGDTVTLSCNYNSSNPSVT |
| | | RYEWKPHGAWEEPSLGVLKIQNVGWDNTTIACAACNSWCSWASPVALNVQYAPRDVRVR |
| | | KIKPLSEIHSGNSVSLQCDFSSSHPKEVQFFWEKNGRLLGKESQLNFDSISPEDAGSYSCWVN |
| | | NSIGQTASKAWTLEVLYAPRRLRVSMSPGDQVMEGKSATLTCESDANPPVSHYTWFDWNN |
| | | QSLPYHSQKLRLEPVKVQHSGAYWCQGTNSVGKGRSPLSTLTVYYSPETIGRRGGGGSGLND |
| | | IFEAQKIEWHEGGGGSHHHHHH |
| 474 | | DSSKWVFEHPETLYAWEGACVWIPCTYRALDGDLESFILFHNPEYNKNTSKFDGTRLYESTK |
| | | DGKVPSEQKRVQFLGDKNKNCTLSIHPVHLNDSGQLGLRMESKTEKWMERIHLNVSERPFPP |
| | | HIQLPPEIQESQEVTLTCLLNFSCYGYPIQLQWLLEGVPMRQAAVTSTSLTIKSVFTRSELKFSP |
| | | QWSHHGKIVTCQLQDADGKFLSNDTVQLNVKHTPKLEIKVTPSDAIVREGDSVTMTCEVSSS |
| | | NPEYTTVSWLKDGTSLKKQNTFTLNLREVTKDQSGKYCCQVSNDVGPGRSEEVFLQVQYAG |
| | | GGGSGLNDIFEAQKIEWHEGGGGSHHHHHH |
| 475 | anti-CD19-B H-CDR1 (Kabat | TKAMG |
| | numbering) | |
| 476 | anti-CD19-B H-CDR2 (Kabat | AITWTAGLTYYADSVKG |
| | numbering) | |
| 477 | anti-CD19-B H-CDR3 (Kabat | RRTFSYEHPNYYTRSAYHF |
| | numbering) | |
| 481 | VHH-B (VHH of CAR #3, | EVQLVESGGGVVQPGGSLRLSCAASGDTFGTKAMGWFRQAPGKGREFVAAITWTAGLTYY |
| | CAR #43, CAR #44, and | ADSVKGRFTISKDRAKNTVDLQMNSLRPEDTALYYCASRRTFSYEHPNYYTRSAYHFWGQG |
| | CAR #47) | TLVTVSS |
| 482 | Anti-CD19 CAR #22 nucleic | ATGGAGACCGACACCCTCCTGCTGTGGGTGCTGCTGCTTTGGGTGCCCGGGAGCACGGGC |
| | acid sequence | GAGGTCCAGCTGGTCGAGAGTGGCGGAGGCCTGGTGCAGCCCGGCGGCAGCCTGAGACT |
| | | GTTCTGCGCCGCCAGCGGCAGCAGGCTGAGCTTCACCACCATGGGCTGGTACCGCCAGG |
| | | CCCCAGGCAAGCAGCGAGAGGTGGTGGCCTATATCACGGAGAGCGGCTCGACCGCCTAC |
| | | ACCGCCAGTGTGAAGGACAGGTTCACCATCTCCCGGGACAACGCCAAGAATACCGTGTC |
| | | CCTCCAGATGAACAGCCTGAAGCCGGAGGACACCGCCGTGTACTTCTGCAACGCGGTGA |
| | | TCATGATGGGCGGGAGCGGCACCTACTGGGGCCCCGGGACGCTCGTGACCGTCAGCTCC |
| | | GCGGCCGCGATAGAGGTAATGTACCCTCCTCCTTACCTGGACAACGAGAAGTCGAACGG |
| | | CACCATCATCCACGTCAAGGGCAAGCACCTCTGCCCGTCGCCGCTGTTCCCGGGCCCGTC |
| | | CAAGCCCTTCTGGGTGCTGGTCGTGGTTGGTGGCGTCCTCGCCTGCTACTCCCTCCTCGTC |
| | | ACGGTGGCGTTCATCATCTTCTGGGTGCGGAGCAAGCGAAGCCGCCTCCTGCACAGCGA |
| | | CTACATGAACATGACGCCGCGCCGCCCGGGCCCGACCCGGAAGCACTACCAGCCCTACG |
| | | CGCCGCCGCGCGACTTCGCGGCCTACCGCTCCCGGGTCAAGTTCTCCCGCAGCGCCGACG |
| | | CGCCCGCGTACCAGCAGGGCCAGAACCAGCTCTACAACGAGCTCAACCTGGGGAGGCGC |
| | | GAGGAGTACGACGTGCTTGACAAGCGCCGTGGCCGGGACCCTGAGATGGGCGGTAAGCC |
| | | GCGACGGAAGAACCCGCAGGAGGGCCTGTACAACGAGCTGCAGAAGGACAAGATGGCG |
| | | GAGGCCTACAGCGAGATCGGGATGAAGGGCGAGCGGCGGCGTGGCAAGGGCCACGACG |
| | | GCCTGTACCAGGGCCTCAGTACGGCCACCAAGGACACCTACGACGCCTTGCACATGCAG |
| | | GCCCTCCCGCCGCGCTAGTGATGA |
| 483 | Anti-CD19 CAR #23 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGTCCGGCGGCGGGCTGGTGCAGGCCGGCGACAGCCTGAGGCT |
| | | CTCGTGCGCCGCCAGCGGCAGGACCCTGAGCAGCTACACGACCGGCTGGTTCCGTCAGG |
| | | GGCTGGGCAAGGAGCGCGAGTTCGTGGCCGCGATCACCAAGAACGGCCCCTACCTCTAC |
| | | TACAAGGACAGCGTCAGGGGCCGCTTCACCATCTCCCGAGACAGCGCCAAGACCACCGT |
| | | GTTCCTGCAGATGGACAACCTGAAGCCGGAGGACACCGCCGTCTATTACTGCGCCGTAA |
| | | GCGACGCCCAGTACGACATCCGGAACCGGGAGAAGTACTACTACTGGGGCCAGGGCACC |
| | | CTGGTGACGGTCTCCAGCGCGGCCGCGATAGAGGTAATGTACCCTCCTCCTTACCTGGAC |
| | | AACGAGAAGTCGAACGGCACCATCATCCACGTCAAGGGCAAGCACCTCTGCCCGTCGCC |
| | | GCTGTTCCCGGGCCCGTCCAAGCCCTTCTGGGTGCTGGTCGTGGTTGGTGGCGTCCTCGC |
| | | CTGCTACTCCCTCCTCGTCACGGTGGCGTTCATCATCTTCTGGGTGCGGAGCAAGCGAAG |
| | | CCGCCTCCTGCACAGCGACTACATGAACATGACGCCGCGCCGCCCGGGCCCGACCCGGA |
| | | AGCACTACCAGCCCTACGCGCCGCCGCGCGACTTCGCGGCCTACCGCTCCCGGGTCAAGT |
| | | TCTCCCGCAGCGCCGACGCGCCCGCGTACCAGCAGGGCCAGAACCAGCTCTACAACGAG |
| | | CTCAACCTGGGGAGGCGCGAGGAGTACGACGTGCTTGACAAGCGCCGTGGCCGGGACCC |
| | | TGAGATGGGCGGTAAGCCGCGACGGAAGAACCCGCAGGAGGGCCTGTACAACGAGCTG |
| | | CAGAAGGACAAGATGGCGGAGGCCTACAGCGAGATCGGGATGAAGGGCGAGCGGCGGC |
| | | GTGGCAAGGGCCACGACGGCCTGTACCAGGGCCTCAGTACGGCCACCAAGGACACCTAC |
| | | GACGCCTTGCACATGCAGGCCCTCCCGCCGCGCTAGTGATGA |
| 484 | Anti-CD19 CAR #24 nucleic | ATGGAGACCGACACCCTCCTGCTGTGGGTGCTGCTGCTTTGGGTGCCCGGGAGCACGGGC |
| | acid sequence | GAGGTCCAGCTGGTGGAGAGCGGCGGCGGCCTGGTGCAGGCCGGCGACAGCCTGCGCCT |
| | | GTCCTGCGCCGCCAGCGGCAGGACCCTGAGCAGCTACACGACCGGCTGGTTCCGACAGG |
| | | GCCTCGGGAAGGAGAGGGAATTCGTCGCCGCCATCACCAAGAACGGCCCGTACCTCTAC |
| | | TACAAGGACTCCGTCCGCGGCCGCTTCACCATCAGCCGGGACTCCGCCAAGACCACCGT |
| | | GTTCCTGCAGATGGACAACCTGAAGCCCGAGGACACCGCGGTGTACTACTGCGCCGTGT |
| | | CGGACGCCCAGTACGACATCCGTAACAGAGAGAAGTACTACTACTGGGGGCAGGGCACC |
| | | CTGGTGACGGTGAGCAGCGCCAGCCTTAGCAACAGCATCATGTACTTCAGCCACTTTGTC |
| | | CCGGTCTTCCTGCCAGCCAAGCCGACCACCACGCCCGCGCCGCGGCCGCCGACGCCGGC |
| | | GCCCACAATCGCCTCACAGCCACTGAGCCTGCGCCCGGAGGCCTGCAGGCCCGCCGCAG |
| | | GCGGCGCCGTGCACACGCGTGGGCTCGACTTCGCCTGTGACATATACATCTGGGCGCCCC |
| | | TGGCCGGCACCTGCGGCGTCCTGCTGCTGAGCCTGGTGATCACCCTGTATTGCAACCACC |
| | | GGAACGTGCGCTCCAAACGCAGCCGGCTGCTGCACAGCGACTACATGAACATGACGCCG |
| | | CGCAGGCCCGGCCCGACCCGAAAGCACTACCAGCCATACGCGCCGCCGCGGGACTTCGC |
| | | CGCCTACCGGTCAAGGGTTAAGTTCTCACGGTCGGCCGACGCGCCCGCCTACCAGCAGG |
| | | GCCAGAACCAGCTGTACAATGAGCTGAACCTGGGCCGGAGGGAGGAGTACGACGTACTG |
| | | GATAAGAGGCGCGGCAGAGACCCCGAGATGGGCGGCAAACCCAGGCGTAAGAACCCGC |
| | | AGGAGGGCCTGTACAACGAACTGCAGAAGGACAAGATGGCCGAGGCCTACAGCGAAAT |
| | | AGGCATGAAGGGCGAGCGTAGGCGCGGGAAGGGCCACGACGGCCTGTATCAAGGCCTG |
| | | TCGACCGCCACCAAAGACACCTACGACGCCCTGCACATGCAGGCCCTGCCGCCCAGGTA |
| | | GTGATGA |
| 485 | Anti-CD19 CAR #25 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGTCCGGCGGCGGGCTGGTGCAGGCCGGCGACAGCCTGAGGCT |
| | | CTCGTGCGCCGCCAGCGGCAGGACCCTGAGCAGCTACACGACCGGCTGGTTCCGTCAGG |
| | | GGCTGGGCAAGGAGCGCGAGTTCGTGGCCGCGATCACCAAGAACGGCCCCTACCTCTAC |
| | | TACAAGGACAGCGTCAGGGGCCGCTTCACCATCTCCCGAGACAGCGCCAAGACCACCGT |
| | | GTTCCTGCAGATGGACAACCTGAAGCCGGAGGACACCGCCGTCTATTACTGCGCCGTAA |
| | | GCGACGCCCAGTACGACATCCGGAACCGGGAGAAGTACTACTACTGGGGCCAGGGCACC |
| | | CTGGTGACGGTCTCCAGCGGAGGCGGCGGCTCCGGAGGCGGAGGCAGCGGCGGCGGCG |
| | | GATCGGAGGTCCAACTGGTGGAGAGCGGAGGCGGACTCGTGCAGGCCGAGGGCTCCCTG |
| | | CGGCTGAGCTGCACCGCATCCGGCCTCACATTCGACAACTACGCCATGGGCTGGTTCCGC |
| | | CAGGCCCCAGGCAAGGAAAGGGAGTTCGTGGCGACGATCAGCGCGGCCGGCCACCGCA |
| | | CCATCTTCACCAACAGCGTGCGCGGAAGGTTCACCGCCAGCCGAGACAACGCCAAGAAG |
| | | ACCACGTACCTGCAGATGAACTCCCTGAAGCCCGAGGACACCGCCGTGTACTACTGTGCC |
| | | GCCAGCCCGAGCCAGTACGGGAGCATGGGCAAGAGCGCCTACGACTACTGGGGCCAGG |
| | | GCACCCTGGTCACCGTGTCGAGCGCGGCCGCGATAGAGGTAATGTACCCTCCTCCTTACC |
| | | TGGACAACGAGAAGTCGAACGGCACCATCATCCACGTCAAGGGCAAGCACCTCTGCCCG |
| | | TCGCCGCTGTTCCCGGGCCCGTCCAAGCCCTTCTGGGTGCTGGTCGTGGTTGGTGGCGTC |
| | | CTCGCCTGCTACTCCCTCCTCGTCACGGTGGCGTTCATCATCTTCTGGGTGCGGAGCAAG |
| | | CGAAGCCGCCTCCTGCACAGCGACTACATGAACATGACGCCGCGCCGCCCGGGCCCGAC |
| | | CCGGAAGCACTACCAGCCCTACGCGCCGCCGCGCGACTTCGCGGCCTACCGCTCCCGGGT |
| | | CAAGTTCTCCCGCAGCGCCGACGCGCCCGCGTACCAGCAGGGCCAGAACCAGCTCTACA |
| | | ACGAGCTCAACCTGGGGAGGCGCGAGGAGTACGACGTGCTTGACAAGCGCCGTGGCCGG |
| | | GACCCTGAGATGGGCGGTAAGCCGCGACGGAAGAACCCGCAGGAGGGCCTGTACAACG |
| | | AGCTGCAGAAGGACAAGATGGCGGAGGCCTACAGCGAGATCGGGATGAAGGGCGAGCG |
| | | GCGGCGTGGCAAGGGCCACGACGGCCTGTACCAGGGCCTCAGTACGGCCACCAAGGACA |
| | | CCTACGACGCCTTGCACATGCAGGCCCTCCCGCCGCGCTAGTGATGA |
| 486 | Anti-CD19 CAR #26 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGTCCGGCGGCGGGCTGGTGCAGGCCGGCGACAGCCTGAGGCT |
| | | CTCGTGCGCCGCCAGCGGCAGGACCCTGAGCAGCTACACGACCGGCTGGTTCCGTCAGG |
| | | GGCTGGGCAAGGAGCGCGAGTTCGTGGCCGCGATCACCAAGAACGGCCCCTACCTCTAC |
| | | TACAAGGACAGCGTCAGGGGCCGCTTCACCATCTCCCGAGACAGCGCCAAGACCACCGT |
| | | GTTCCTGCAGATGGACAACCTGAAGCCGGAGGACACCGCCGTCTATTACTGCGCCGTAA |
| | | GCGACGCCCAGTACGACATCCGGAACCGGGAGAAGTACTACTACTGGGGCCAGGGCACC |
| | | CTGGTGACGGTCTCCAGCGGAGGCGGCGGCTCCGGAGGCGGAGGCAGCGGCGGCGGCG |
| | | GATCGGAGGTGCAGCTCGTCGAGAGCGGCGGCGGCCTGGTACAGACCGGCGACAGCCTC |
| | | CGTCTGACCTGCGCCGCCAGCGGCCGCACCATCGGCGACCGGTCCATGGCGTGGTTCAG |
| | | GCAGGCCCCGGGCAAGGAGCGGGAGTTCGTCATCGTCAACACGTGGAACGGCTACACCT |
| | | ACACCGACGAGAGCGTGGAGGGCCGCTTCACCATCAGCAGGGACAGCGGCGCCAACAC |
| | | GGTGCACCTGCAGATGAACAAGCTGAACCCCAAGGACACCGCCGTGTACTACTGCGCCT |
| | | CCTCCAGCTACACCTATAACATCAGGCAGCGAAGCTCGTACGACTGGTGGGGCCAGGGG |
| | | ACGCTGGTGACCGTGAGCAGCGCGGCCGCGATAGAGGTAATGTACCCTCCTCCTTACCTG |
| | | GACAACGAGAAGTCGAACGGCACCATCATCCACGTCAAGGGCAAGCACCTCTGCCCGTC |
| | | GCCGCTGTTCCCGGGCCCGTCCAAGCCCTTCTGGGTGCTGGTCGTGGTTGGTGGCGTCCT |
| | | CGCCTGCTACTCCCTCCTCGTCACGGTGGCGTTCATCATCTTCTGGGTGCGGAGCAAGCG |
| | | AAGCCGCCTCCTGCACAGCGACTACATGAACATGACGCCGCGCCGCCCGGGCCCGACCC |
| | | GGAAGCACTACCAGCCCTACGCGCCGCCGCGCGACTTCGCGGCCTACCGCTCCCGGGTC |
| | | AAGTTCTCCCGCAGCGCCGACGCGCCCGCGTACCAGCAGGGCCAGAACCAGCTCTACAA |
| | | CGAGCTCAACCTGGGGAGGCGCGAGGAGTACGACGTGCTTGACAAGCGCCGTGGCCGGG |
| | | ACCCTGAGATGGGCGGTAAGCCGCGACGGAAGAACCCGCAGGAGGGCCTGTACAACGA |
| | | GCTGCAGAAGGACAAGATGGCGGAGGCCTACAGCGAGATCGGGATGAAGGGCGAGCGG |
| | | CGGCGTGGCAAGGGCCACGACGGCCTGTACCAGGGCCTCAGTACGGCCACCAAGGACAC |
| | | CTACGACGCCTTGCACATGCAGGCCCTCCCGCCGCGCTAGTGATGA |
| 487 | Anti-CD19 CAR #27 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGTCCGGCGGCGGCCTGGTGCCGCCGGGCGGCTCCCTCAGGCT |
| | | CAGTTGCGCGGCCAGCGGGCGGACGGGAGACATCAACGGCATGGGCTGGTTCAGGCGCG |
| | | CCGCCGGCAAAGAAAGAGAGTTCGTGGCCGCCACGCGGTGGAACGGCGGCACCAAGTA |
| | | CTACACCAGCAGCGTCGAGGGCAGATTCACCATAAGCGGCGACAGGGTCGGAAACACCG |
| | | TGACCCTGGAAATGAATAACCTGGTGCCCGAGGACACGGCCCGCTATTACTGTGCAACC |
| | | ACCCGACGTCGGTTCGACTTCTATAGCGTCGGCGGGTACGAGAACTGGGGCCAGGGCAC |
| | | CCAGGTGACCGTGAGCAGCGCCAGCCTTAGCAACAGCATCATGTACTTCAGCCACTTTGT |
| | | CCCGGTCTTCCTGCCAGCCAAGCCGACCACCACGCCCGCGCCGCGGCCGCCGACGCCGG |
| | | CGCCCACAATCGCCTCACAGCCACTGAGCCTGCGCCCGGAGGCCTGCAGGCCCGCCGCA |
| | | GGCGGCGCCGTGCACACGCGTGGGCTCGACTTCGCCTGTGACATATACATCTGGGCGCCC |
| | | CTGGCCGGCACCTGCGGCGTCCTGCTGCTGAGCCTGGTGATCACCCTGTATTGCAACCAC |
| | | CGGAACGTGCGCTCCAAACGCAGCCGGCTGCTGCACAGCGACTACATGAACATGACGCC |
| | | GCGCAGGCCCGGCCCGACCCGAAAGCACTACCAGCCATACGCGCCGCCGCGGGACTTCG |
| | | CCGCCTACCGGTCAAGGGTTAAGTTCTCACGGTCGGCCGACGCGCCCGCCTACCAGCAG |
| | | GGCCAGAACCAGCTGTACAATGAGCTGAACCTGGGCCGGAGGGAGGAGTACGACGTACT |
| | | GGATAAGAGGCGCGGCAGAGACCCCGAGATGGGCGGCAAACCCAGGCGTAAGAACCCG |
| | | CAGGAGGGCCTGTACAACGAACTGCAGAAGGACAAGATGGCCGAGGCCTACAGCGAAA |
| | | TAGGCATGAAGGGCGAGCGTAGGCGCGGGAAGGGCCACGACGGCCTGTATCAAGGCCT |
| | | GTCGACCGCCACCAAAGACACCTACGACGCCCTGCACATGCAGGCCCTGCCGCCCAGGT |
| | | AGTGATGA |
| 488 | Anti-CD19 CAR #28 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGAGCGGCGGCGGGCTCGTGCCGCCCGGCGGGAGCCTACGGCT |
| | | GAGCTGCGCCGCATCCGGCCGGACCGGAGACATCAACGGCATGGGCTGGTTTCGCCGAG |
| | | CCGCAGGGAAGGAACGGGAGTTCGTCGCCGCCACGCGTTGGAACGGCGGAACCAAATAC |
| | | TATACAAGCAGTGTCGAGGGCAGGTTCACCATCTCCGGGGACCGAGTGGGCAACACCGT |
| | | GACCCTGGAGATGAACAACCTCGTGCCGGAGGATACGGCCAGGTACTACTGCGCGACCA |
| | | CCCGCCGCAGGTTCGATTTCTACAGCGTGGGCGGATACGAGAACTGGGGCCAGGGCACC |
| | | CAGGTGACCGTCAGCAGCGCGAGCACCACCACGCCCGCGCCGCGGCCGCCGACGCCGGC |
| | | GCCCACAATCGCCTCACAGCCACTGAGCCTGCGCCCGGAGGCCTGCAGGCCCGCCGCAG |
| | | GCGGCGCCGTGCACACGCGTGGGCTCGACTTCGCCTGTGACATATACATCTGGGCGCCCC |
| | | TGGCCGGCACCTGCGGCGTCCTGCTGCTGAGCCTGGTGATCACCCTGTATTGCAACCACC |
| | | GGAACGTGCGCTCCAAACGCAGCCGGCTGCTGCACAGCGACTACATGAACATGACGCCG |
| | | CGCAGGCCCGGCCCGACCCGAAAGCACTACCAGCCATACGCGCCGCCGCGGGACTTCGC |
| | | CGCCTACCGGTCAAGGGTTAAGTTCTCACGGTCGGCCGACGCGCCCGCCTACCAGCAGG |
| | | GCCAGAACCAGCTGTACAATGAGCTGAACCTGGGCCGGAGGGAGGAGTACGACGTACTG |
| | | GATAAGAGGCGCGGCAGAGACCCCGAGATGGGCGGCAAACCCAGGCGTAAGAACCCGC |
| | | AGGAGGGCCTGTACAACGAACTGCAGAAGGACAAGATGGCCGAGGCCTACAGCGAAAT |
| | | AGGCATGAAGGGCGAGCGTAGGCGCGGGAAGGGCCACGACGGCCTGTATCAAGGCCTG |
| | | TCGACCGCCACCAAAGACACCTACGACGCCCTGCACATGCAGGCCCTGCCGCCCAGGTA |
| | | GTGATGA |
| 489 | Anti-CD19 CAR #29 nucleic | ATGGAGACCGACACCCTCCTGCTGTGGGTGCTGCTGCTTTGGGTGCCCGGGAGCACGGGC |
| | acid sequence | GAGGTCCAGCTGGTGGAGAGCGGCGGCGGGCTCGTGCAGCCCGGCGGGAGCCTCAGTCT |
| | | GAGCTGCGCCGCCAGCGGAGACACCTTCGGGACCAAGGCCATGGGCTGGTTCAGGCAGG |
| | | CGCCGGGCAAGGGCCGAGAATTCGTGGCCGCCATCACGTGGACCGCCGGCCTGACCTAC |
| | | CTGGCGGACTCCGCCAAGGGCCGGTTTACCATCTCCAAGGACCGCGTGCAGAACACCGT |
| | | CTACCTGGAGATGAACAACCTGGAGCCCGAGGACACAGCCGTCTACTACTGCGCCTCGC |
| | | GGAGGACCTTCAGCTACGAACACCCCAACTACTACACCGCCTCCGCGTACCACTTCTGGG |
| | | GACAGGGCACGCTGGTGACCGTGAGCAGCGCGGCCGCGATAGAGGTAATGTACCCTCCT |
| | | CCTTACCTGGACAACGAGAAGTCGAACGGCACCATCATCCACGTCAAGGGCAAGCACCT |
| | | CTGCCCGTCGCCGCTGTTCCCGGGCCCGTCCAAGCCCTTCTGGGTGCTGGTCGTGGTTGG |
| | | TGGCGTCCTCGCCTGCTACTCCCTCCTCGTCACGGTGGCGTTCATCATCTTCTGGGTGCGG |
| | | AGCAAGCGAAGCCGCCTCCTGCACAGCGACTACATGAACATGACGCCGCGCCGCCCGGG |
| | | CCCGACCCGGAAGCACTACCAGCCCTACGCGCCGCCGCGCGACTTCGCGGCCTACCGCTC |
| | | CCGGGTCAAGTTCTCCCGCAGCGCCGACGCGCCCGCGTACCAGCAGGGCCAGAACCAGC |
| | | TCTACAACGAGCTCAACCTGGGGAGGCGCGAGGAGTACGACGTGCTTGACAAGCGCCGT |
| | | GGCCGGGACCCTGAGATGGGCGGTAAGCCGCGACGGAAGAACCCGCAGGAGGGCCTGT |
| | | ACAACGAGCTGCAGAAGGACAAGATGGCGGAGGCCTACAGCGAGATCGGGATGAAGGG |
| | | CGAGCGGCGGCGTGGCAAGGGCCACGACGGCCTGTACCAGGGCCTCAGTACGGCCACCA |
| | | AGGACACCTACGACGCCTTGCACATGCAGGCCCTCCCGCCGCGCTAGTGATGA |
| 490 | Anti-CD19 CAR #30 nucleic | ATGGAGACCGACACCCTCCTGCTGTGGGTGCTGCTGCTTTGGGTGCCCGGGAGCACGGGC |
| | acid sequence | GAGGTCCAGCTGGTGGAGAGCGGCGGCGGGCTCGTGCAGCCCGGCGGGAGCCTCAGTCT |
| | | GAGCTGCGCCGCCAGCGGAGACACCTTCGGGACCAAGGCCATGGGCTGGTTCAGGCAGG |
| | | CGCCGGGCAAGGGCCGAGAATTCGTGGCCGCCATCACGTGGACCGCCGGCCTGACCTAC |
| | | CTGGCGGACTCCGCCAAGGGCCGGTTTACCATCTCCAAGGACCGCGTGCAGAACACCGT |
| | | CTACCTGGAGATGAACAACCTGGAGCCCGAGGACACAGCCGTCTACTACTGCGCCTCGC |
| | | GGAGGACCTTCAGCTACGAACACCCCAACTACTACACCGCCTCCGCGTACCACTTCTGGG |
| | | GACAGGGCACGCTGGTGACCGTGAGCAGCGGAGGCGGCGGCTCCGGAGGCGGAGGCAG |
| | | CGGCGGCGGCGGATCGGAGGTCCAACTGGTGGAGAGCGGAGGCGGACTCGTGCAGGCC |
| | | GAGGGCTCCCTGCGGCTGAGCTGCACCGCATCCGGCCTCACATTCGACAACTACGCCATG |
| | | GGCTGGTTCCGCCAGGCCCCAGGCAAGGAAAGGGAGTTCGTGGCGACGATCAGCGCGGC |
| | | CGGCCACCGCACCATCTTCACCAACAGCGTGCGCGGAAGGTTCACCGCCAGCCGAGACA |
| | | ACGCCAAGAAGACCACGTACCTGCAGATGAACTCCCTGAAGCCCGAGGACACCGCCGTG |
| | | TACTACTGTGCCGCCAGCCCGAGCCAGTACGGGAGCATGGGCAAGAGCGCCTACGACTA |
| | | CTGGGGCCAGGGCACCCTGGTCACCGTGTCGAGCGCGGCCGCGATAGAGGTAATGTACC |
| | | CTCCTCCTTACCTGGACAACGAGAAGTCGAACGGCACCATCATCCACGTCAAGGGCAAG |
| | | CACCTCTGCCCGTCGCCGCTGTTCCCGGGCCCGTCCAAGCCCTTCTGGGTGCTGGTCGTG |
| | | GTTGGTGGCGTCCTCGCCTGCTACTCCCTCCTCGTCACGGTGGCGTTCATCATCTTCTGGG |
| | | TGCGGAGCAAGCGAAGCCGCCTCCTGCACAGCGACTACATGAACATGACGCCGCGCCGC |
| | | CCGGGCCCGACCCGGAAGCACTACCAGCCCTACGCGCCGCCGCGCGACTTCGCGGCCTA |
| | | CCGCTCCCGGGTCAAGTTCTCCCGCAGCGCCGACGCGCCCGCGTACCAGCAGGGCCAGA |
| | | ACCAGCTCTACAACGAGCTCAACCTGGGGAGGCGCGAGGAGTACGACGTGCTTGACAAG |
| | | CGCCGTGGCCGGGACCCTGAGATGGGCGGTAAGCCGCGACGGAAGAACCCGCAGGAGG |
| | | GCCTGTACAACGAGCTGCAGAAGGACAAGATGGCGGAGGCCTACAGCGAGATCGGGAT |
| | | GAAGGGCGAGCGGCGGCGTGGCAAGGGCCACGACGGCCTGTACCAGGGCCTCAGTACG |
| | | GCCACCAAGGACACCTACGACGCCTTGCACATGCAGGCCGTCCCGCCGCGCTAGTGATG |
| | | A |
| 491 | Anti-CD19 CAR #31 nucleic | ATGGAGACCGACACCCTCCTGCTGTGGGTGCTGCTGCTTTGGGTGCCCGGGAGCACGGGC |
| | acid sequence | GAGGTGCAACTCGTGGAGTCCGGCGGCGGCCTGGTGCAGCCGGGCGGGAGCCTGCGGCT |
| | | GTCGTGCGTGGCCAGCGGCCTGTTCCTGAGCGACTACACGCTGGCCTGGTTCCGGCAGGC |
| | | GCCCGGCAAGGAGAGGGAGTTCGTGATCGTGAGCACCTGGAACGGCTACACCTACTACG |
| | | GCGAGAGCGTCTCCGGCAGGTTCACCATCAGCCGCGACAACGCCGAGAAGACCGTGTTC |
| | | CTGCAGATGAACAGCCTGAAGCCCGAGGACACCGCCGTCTACTACTGCGCCGCCAAGTT |
| | | CCGAATCAGCTACAACGTGCGCGACGACAGCTTCTACGACCTCTGGGGCCAGGGCACCC |
| | | TCGTGACCGTCTCCAGCGCGGCCGCGATAGAGGTAATGTACCCTCCTCCTTACCTGGACA |
| | | ACGAGAAGTCGAACGGCACCATCATCCACGTCAAGGGCAAGCACCTCTGCCCGTCGCCG |
| | | CTGTTCCCGGGCCCGTCCAAGCCCTTCTGGGTGCTGGTCGTGGTTGGTGGCGTCCTCGCCT |
| | | GCTACTCCCTCCTCGTCACGGTGGCGTTCATCATCTTCTGGGTGCGGAGCAAGCGAAGCC |
| | | GCCTCCTGCACAGCGACTACATGAACATGACGCCGCGCCGCCCGGGCCCGACCCGGAAG |
| | | CACTACCAGCCCTACGCGCCGCCGCGCGACTTCGCGGCCTACCGCTCCCGGGTCAAGTTC |
| | | TCCCGCAGCGCCGACGCGCCCGCGTACCAGCAGGGCCAGAACCAGCTCTACAACGAGCT |
| | | CAACCTGGGGAGGCGCGAGGAGTACGACGTGCTTGACAAGCGCCGTGGCCGGGACCCTG |
| | | AGATGGGCGGTAAGCCGCGACGGAAGAACCCGCAGGAGGGCCTGTACAACGAGCTGCA |
| | | GAAGGACAAGATGGCGGAGGCCTACAGCGAGATCGGGATGAAGGGCGAGCGGCGGCGT |
| | | GGCAAGGGCCACGACGGCCTGTACCAGGGCCTCAGTACGGCCACCAAGGACACCTACGA |
| | | CGCCTTGCACATGCAGGCCCTCCCGCCGCGCTAGTGATGA |
| 492 | Anti-CD19 CAR #32 nucleic | ATGGAGACCGACACCCTCCTGCTGTGGGTGCTGCTGCTTTGGGTGCCCGGGAGCACGGGC |
| | acid sequence | GAGGTCCAGCTGGTCGAGTCGGGCGGCGGCCTGGTGCAGCCGGGCGGCTCCCTGCGGCT |
| | | GTCCTGCGTGGCCAGCGGGCTGTTCCTCAGCGACTACACCCTGGCCTGGTTCAGGCAGGC |
| | | CCCCGGGAAGGAGCGCGAGTTCGTGATCGTGAGCACCTGGAACGGCTACACCTACTACG |
| | | GCGAGTCCGTGAGCGGCAGGTTCACCATCAGCCGCGACAACGCCGAGAAGACCGTGTTC |
| | | CTGCAGATGAACAGCCTGAAGCCCGAGGACACCGCCGTGTACTACTGCGCGGCGAAGTT |
| | | CCGAATCAGCTACAACGTGCGGGACGACAGCTTCTACGACCTCTGGGGCCAGGGCACCC |
| | | TCGTGACGGTCAGCAGCGCGAGCACCACCACGCCCGCGCCGCGGCCGCCGACGCCGGCG |
| | | CCCACAATCGCCTCACAGCCACTGAGCCTGCGCCCGGAGGCCTGCAGGCCCGCCGCAGG |
| | | CGGCGCCGTGCACACGCGTGGGCTCGACTTCGCCTGTGACATATACATCTGGGCGCCCCT |
| | | GGCCGGCACCTGCGGCGTCCTGCTGCTGAGCCTGGTGATCACCCTGTATTGCAACCACCG |
| | | GAACAAGCGCGGCAGGAAGAAGCTCCTGTACATCTTCAAGCAGCCGTTCATGCGCCCGG |
| | | TGCAGACGACCCAGGAGGAGGACGGGTGCAGCTGCCGGTTCCCGGAGGAGGAGGAGGG |
| | | CGGCTGCGAGCTGAGGGTTAAGTTCTCACGGTCGGCCGACGCGCCCGCCTACCAGCAGG |
| | | GCCAGAACCAGCTGTACAATGAGCTGAACCTGGGCCGGAGGGAGGAGTACGACGTACTG |
| | | GATAAGAGGCGCGGCAGAGACCCCGAGATGGGCGGCAAACCCAGGCGTAAGAACCCGC |
| | | AGGAGGGCCTGTACAACGAACTGCAGAAGGACAAGATGGCCGAGGCCTACAGCGAAAT |
| | | AGGCATGAAGGGCGAGCGTAGGCGCGGGAAGGGCCACGACGGCCTGTATCAAGGCCTG |
| | | TCGACCGCCACCAAAGACACCTACGACGCCCTGCACATGCAGGCCCIGCCGCCCAGGTA |
| | | GTGATGA |
| 493 | Anti-CD19 CAR #33 nucleic | ATGGAGACCGACACCCTCCTGCTGTGGGTGCTGCTGCTTTGGGTGCCCGGGAGCACGGGC |
| | acid sequence | GAGGTCCAGCTGGTCGAGTCGGGCGGCGGCCTGGTGCAGCCGGGCGGCTCCCTGCGGCT |
| | | GTCCTGCGTGGCCAGCGGGCTGTTCCTCAGCGACTACACCCTGGCCTGGTTCAGGCAGGC |
| | | CCCCGGGAAGGAGCGCGAGTTCGTGATCGTGAGCACCTGGAACGGCTACACCTACTACG |
| | | GCGAGTCCGTGAGCGGCAGGTTCACCATCAGCCGCGACAACGCCGAGAAGACCGTGTTC |
| | | CTGCAGATGAACAGCCTGAAGCCCGAGGACACCGCCGTGTACTACTGCGCGGCGAAGTT |
| | | CCGAATCAGCTACAACGTGCGGGACGACAGCTTCTACGACCTCTGGGGCCAGGGCACCC |
| | | TCGTGACGGTCAGCAGCGCGAGCACCACCACGCCCGCGCCGCGGCCGCCGACGCCGGCG |
| | | CCCACAATCGCCTCACAGCCACTGAGCCTGCGCCCGGAGGCCTGCAGGCCCGCCGCAGG |
| | | CGGCGCCGTGCACACGCGTGGGCTCGACTTCGCCTGTGACATATACATCTGGGCGCCCCT |
| | | GGCCGGCACCTGCGGCGTCCTGCTGCTGAGCCTGGTGATCACCCTGTATTGCAACCACCG |
| | | GAACGTGCGCTCCAAACGCAGCCGGCTGCTGCACAGCGACTACATGAACATGACGCCGC |
| | | GCAGGCCCGGCCCGACCCGAAAGCACTACCAGCCATACGCGCCGCCGCGGGACTTCGCC |
| | | GCCTACCGGTCAAGGGTTAAGTTCTCACGGTCGGCCGACGCGCCCGCCTACCAGCAGGG |
| | | CCAGAACCAGCTGTACAATGAGCTGAACCTGGGCCGGAGGGAGGAGTACGACGTACTGG |
| | | ATAAGAGGCGCGGCAGAGACCCCGAGATGGGCGGCAAACCCAGGCGTAAGAACCCGCA |
| | | GGAGGGCCTGTACAACGAACTGCAGAAGGACAAGATGGCCGAGGCCTACAGCGAAATA |
| | | GGCATGAAGGGCGAGCGTAGGCGCGGGAAGGGCCACGACGGCCTGTATCAAGGCCTGTC |
| | | GACCGCCACCAAAGACACCTACGACGCCCTGCACATGCAGGCCCTGCCGCCCAGGTAGT |
| | | GATGA |
| 494 | Anti-CD19 CAR #34 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGAGCGGAGGCGGACTGGTGCAGGCGGGCGGCAGCCTGACGC |
| | | TCACCTGTGCCGCCTCCGGCCTCACCTTCGCAGGATATAGCGTCGGGTGGTTCCGGCAGG |
| | | CCCCAGGCAAGGAGAGGGAGTTCGTGGCCGTGATCACCCGTGGCGGAGACTACCGCTAC |
| | | TTCGCCGAGAGCGTGCAGGGCCGGTTCACCGTGAGCCGAGATAACGGGAAGAACACCGT |
| | | CAGCCTGCAGATGCTGGACCTGAAGCCCGACGACACCGCCGTCTACTACTGCGCCGCAG |
| | | GCACCATGACCTACGCAATCCGCGAGACGAGGGCGTACAACTACTGGGGCCAAGGGACC |
| | | CTGGTGACCGTGAGTTCCGCGAGCACCACCACGCCCGCGCCGCGGCCGCCGACGCCGGC |
| | | GCCCACAATCGCCTCACAGCCACTGAGCCTGCGCCCGGAGGCCTGCAGGCCCGCCGCAG |
| | | GCGGCGCCGTGCACACGCGTGGGCTCGACTTCGCCTGTGACATATACATCTGGGCGCCCC |
| | | TGGCCGGCACCTGCGGCGTCCTGCTGCTGAGCCTGGTGATCACCCTGTATTGCAACCACC |
| | | GGAACGTGCGCTCCAAACGCAGCCGGCTGCTGCACAGCGACTACATGAACATGACGCCG |
| | | CGCAGGCCCGGCCCGACCCGAAAGCACTACCAGCCATACGCGCCGCCGCGGGACTTCGC |
| | | CGCCTACCGGTCAAGGGTTAAGTTCTCACGGTCGGCCGACGCGCCCGCCTACCAGCAGG |
| | | GCCAGAACCAGCTGTACAATGAGCTGAACCTGGGCCGGAGGGAGGAGTACGACGTACTG |
| | | GATAAGAGGCGCGGCAGAGACCCCGAGATGGGCGGCAAACCCAGGCGTAAGAACCCGC |
| | | AGGAGGGCCTGTACAACGAACTGCAGAAGGACAAGATGGCCGAGGCCTACAGCGAAAT |
| | | AGGCATGAAGGGCGAGCGTAGGCGCGGGAAGGGCCACGACGGCCTGTATCAAGGCCTG |
| | | TCGACCGCCACCAAAGACACCTACGACGCCCTGCACATGCAGGCCCTGCCGCCCAGGTA |
| | | GTGATGA |
| 495 | Anti-CD19 CAR #35 nucleic | ATGGAGACCGACACCCTCCTGCTGTGGGTGCTGCTGCTTTGGGTGCCCGGGAGCACGGGC |
| | acid sequence | GAGGTGCAGCTCGTCGAGAGCGGCGGCGGCCTGGTACAGACCGGCGACAGCCTCCGTCT |
| | | GACCTGCGCCGCCAGCGGCCGCACCATCGGCGACCGGTCCATGGCGTGGTTCAGGCAGG |
| | | CCCCGGGCAAGGAGCGGGAGTTCGTCATCGTCAACACGTGGAACGGCTACACCTACACC |
| | | GACGAGAGCGTGGAGGGCCGCTTCACCATCAGCAGGGACAGCGGCGCCAACACGGTGC |
| | | ACCTGCAGATGAACAAGCTGAACCCCAAGGACACCGCCGTGTACTACTGCGCCTCCTCC |
| | | AGCTACACCTATAACATCAGGCAGCGAAGCTCGTACGACTGGTGGGGCCAGGGGACGCT |
| | | GGTGACCGTGAGCAGCGCGGCCGCGATAGAGGTAATGTACCCTCCTCCTTACCTGGACA |
| | | ACGAGAAGTCGAACGGCACCATCATCCACGTCAAGGGCAAGCACCTCTGCCCGTCGCCG |
| | | CTGTTCCCGGGCCCGTCCAAGCCCTTCTGGGTGCTGGTCGTGGTTGGTGGCGTCCTCGCCT |
| | | GCTACTCCCTCCTCGTCACGGTGGCGTTCATCATCTTCTGGGTGCGGAGCAAGCGAAGCC |
| | | GCCTCCTGCACAGCGACTACATGAACATGACGCCGCGCCGCCCGGGCCCGACCCGGAAG |
| | | CACTACCAGCCCTACGCGCCGCCGCGCGACTTCGCGGCCTACCGCTCCCGGGTCAAGTTC |
| | | TCCCGCAGCGCCGACGCGCCCGCGTACCAGCAGGGCCAGAACCAGCTCTACAACGAGCT |
| | | CAACCTGGGGAGGCGCGAGGAGTACGACGTGCTTGACAAGCGCCGTGGCCGGGACCCTG |
| | | AGATGGGCGGTAAGCCGCGACGGAAGAACCCGCAGGAGGGCCTGTACAACGAGCTGCA |
| | | GAAGGACAAGATGGCGGAGGCCTACAGCGAGATCGGGATGAAGGGCGAGCGGCGGCGT |
| | | GGCAAGGGCCACGACGGCCTGTACCAGGGCCTCAGTACGGCCACCAAGGACACCTACGA |
| | | CGCCTTGCACATGCAGGCCCTCCCGCCGCGCTAGTGATGA |
| 496 | Anti-CD19 CAR #36 nucleic | ATGGAGACCGACACCCTCCTGCTGTGGGTGCTGCTGCTTTGGGTGCCCGGGAGCACGGGC |
| | acid sequence | GAGGTGCAGCTCGTCGAGAGCGGCGGCGGCCTGGTACAGACCGGCGACAGCCTCCGTCT |
| | | GACCTGCGCCGCCAGCGGCCGCACCATCGGCGACCGGTCCATGGCGTGGTTCAGGCAGG |
| | | CCCCGGGCAAGGAGCGGGAGTTCGTCATCGTCAACACGTGGAACGGCTACACCTACACC |
| | | GACGAGAGCGTGGAGGGCCGCTTCACCATCAGCAGGGACAGCGGCGCCAACACGGTGC |
| | | ACCTGCAGATGAACAAGCTGAACCCCAAGGACACCGCCGTGTACTACTGCGCCTCCTCC |
| | | AGCTACACCTATAACATCAGGCAGCGAAGCTCGTACGACTGGTGGGGCCAGGGGACGCT |
| | | GGTGACCGTGAGCAGCGGAGGCGGCGGCTCCGGAGGCGGAGGCAGCGGCGGCGGCGGA |
| | | TCGGAGGTCCAACTGGTGGAGAGCGGAGGCGGACTCGTGCAGGCCGAGGGCTCCCTGCG |
| | | GCTGAGCTGCACCGCATCCGGCCTCACATTCGACAACTACGCCATGGGCTGGTTCCGCCA |
| | | GGCCCCAGGCAAGGAAAGGGAGTTCGTGGCGACGATCAGCGCGGCCGGCCACCGCACC |
| | | ATCTTCACCAACAGCGTGCGCGGAAGGTTCACCGCCAGCCGAGACAACGCCAAGAAGAC |
| | | CACGTACCTGCAGATGAACTCCCTGAAGCCCGAGGACACCGCCGTGTACTACTGTGCCGC |
| | | CAGCCCGAGCCAGTACGGGAGCATGGGCAAGAGCGCCTACGACTACTGGGGCCAGGGC |
| | | ACCCTGGTCACCGTGTCGAGCGCGGCCGCGATAGAGGTAATGTACCCTCCTCCTTACCTG |
| | | GACAACGAGAAGTCGAACGGCACCATCATCCACGTCAAGGGCAAGCACCTCTGCCCGTC |
| | | GCCGCTGTTCCCGGGCCCGTCCAAGCCCTTCTGGGTGCTGGTCGTGGTTGGTGGCGTCCT |
| | | CGCCTGCTACTCCCTCCTCGTCACGGTGGCGTTCATCATCTTCTGGGTGCGGAGCAAGCG |
| | | AAGCCGCCTCCTGCACAGCGACTACATGAACATGACGCCGCGCCGCCCGGGCCCGACCC |
| | | GGAAGCACTACCAGCCCTACGCGCCGCCGCGCGACTTCGCGGCCTACCGCTCCCGGGTC |
| | | AAGTTCTCCCGCAGCGCCGACGCGCCCGCGTACCAGCAGGGCCAGAACCAGCTCTACAA |
| | | CGAGCTCAACCTGGGGAGGCGCGAGGAGTACGACGTGCTTGACAAGCGCCGTGGCCGGG |
| | | ACCCTGAGATGGGCGGTAAGCCGCGACGGAAGAACCCGCAGGAGGGCCTGTACAACGA |
| | | GCTGCAGAAGGACAAGATGGCGGAGGCCTACAGCGAGATCGGGATGAAGGGCGAGCGG |
| | | CGGCGTGGCAAGGGCCACGACGGCCTGTACCAGGGCCTCAGTACGGCCACCAAGGACAC |
| | | CTACGACGCCTTGCACATGCAGGCCCTCCCGCCGCGCTAGTGATGA |
| 497 | Anti-CD19 CAR #37 nucleic | ATGGAGACCGACACCCTCCTGCTGTGGGTGCTGCTGCTTTGGGTGCCCGGGAGCACGGGC |
| | acid sequence | GAGGTGCAGCTCGTCGAGAGCGGCGGCGGCCTGGTACAGACCGGCGACAGCCTCCGTCT |
| | | GACCTGCGCCGCCAGCGGCCGCACCATCGGCGACCGGTCCATGGCGTGGTTCAGGCAGG |
| | | CCCCGGGCAAGGAGCGGGAGTTCGTCATCGTCAACACGTGGAACGGCTACACCTACACC |
| | | GACGAGAGCGTGGAGGGCCGCTTCACCATCAGCAGGGACAGCGGCGCCAACACGGTGC |
| | | ACCTGCAGATGAACAAGCTGAACCCCAAGGACACCGCCGTGTACTACTGCGCCTCCTCC |
| | | AGCTACACCTATAACATCAGGCAGCGAAGCTCGTACGACTGGTGGGGCCAGGGGACGCT |
| | | GGTGACCGTGAGCAGCGGAGGCGGCGGCTCCGGAGGCGGAGGCAGCGGCGGCGGCGGA |
| | | TCGGAGGTGCAGCTGGTGGAGTCCGGCGGCGGGCTGGTGCAGGCCGGCGACAGCCTGAG |
| | | GCTCTCGTGCGCCGCCAGCGGCAGGACCCTGAGCAGCTACACGACCGGCTGGTTCCGTC |
| | | AGGGGCTGGGCAAGGAGCGCGAGTTCGTGGCCGCGATCACCAAGAACGGCCCCTACCTC |
| | | TACTACAAGGACAGCGTCAGGGGCCGCTTCACCATCTCCCGAGACAGCGCCAAGACCAC |
| | | CGTGTTCCTGCAGATGGACAACCTGAAGCCGGAGGACACCGCCGTCTATTACTGCGCCGT |
| | | AAGCGACGCCCAGTACGACATCCGGAACCGGGAGAAGTACTACTACTGGGGCCAGGGCA |
| | | CCCTGGTGACGGTCTCCAGCGCGGCCGCGATAGAGGTAATGTACCCTCCTCCTTACCTGG |
| | | ACAACGAGAAGTCGAACGGCACCATCATCCACGTCAAGGGCAAGCACCTCTGCCCGTCG |
| | | CCGCTGTTCCCGGGCCCGTCCAAGCCCTTCTGGGTGCTGGTCGTGGTTGGTGGCGTCCTC |
| | | GCCTGCTACTCCCTCCTCGTCACGGTGGCGTTCATCATCTTCTGGGTGCGGAGCAAGCGA |
| | | AGCCGCCTCCTGCACAGCGACTACATGAACATGACGCCGCGCCGCCCGGGCCCGACCCG |
| | | GAAGCACTACCAGCCCTACGCGCCGCCGCGCGACTTCGCGGCCTACCGCTCCCGGGTCA |
| | | AGTTCTCCCGCAGCGCCGACGCGCCCGCGTACCAGCAGGGCCAGAACCAGCTCTACAAC |
| | | GAGCTCAACCTGGGGAGGCGCGAGGAGTACGACGTGCTTGACAAGCGCCGTGGCCGGGA |
| | | CCCTGAGATGGGCGGTAAGCCGCGACGGAAGAACCCGCAGGAGGGCCTGTACAACGAG |
| | | CTGCAGAAGGACAAGATGGCGGAGGCCTACAGCGAGATCGGGATGAAGGGCGAGCGGC |
| | | GGCGTGGCAAGGGCCACGACGGCCTGTACCAGGGCCTCAGTACGGCCACCAAGGACACC |
| | | TACGACGCCTTGCACATGCAGGCCCTCCCGCCGCGCTAGTGATGA |
| 498 | Anti-CD19 CAR #38 nucleic | ATGGAGACCGACACCCTCCTGCTGTGGGTGCTGCTGCTTTGGGTGCCCGGGAGCACGGGC |
| | acid sequence | GAGGTCCAGCTGGTCGAGTCCGGCGGCGGCCAGGTGCAGGCCGGGGGCAGCCTGCGCCT |
| | | CAGCTGCGTGCACTCGGGACGAACGTTCTCCTCCTACGTGGCAGCGTGGTTTCGGCAGTA |
| | | CCCCGGCAAGGAGAGGGAGTTCGTGGCCGCGGCCAGCCTCAGCGGCGGCACCACCGATT |
| | | ACGCCGACAGCGTGAAGGGCCGGTTCACCATCACAAGGGACAACGCCGCAAGCACGCAC |
| | | TACCTGCAGATGAACAGCCTGCAGCCGGAGGACACCGCCGTCTACTACTGCGCCGCCAG |
| | | ACCCGTGTACCTGAAGTACACCTACCACGATTACGACTTTTGGGGCCAAGGCACCCTGGT |
| | | GACCGTGAGCAGCGCGGCCGCGATAGAGGTAATGTACCCTCCTCCTTACCTGGACAACG |
| | | AGAAGTCGAACGGCACCATCATCCACGTCAAGGGCAAGCACCTCTGCCCGTCGCCGCTG |
| | | TTCCCGGGCCCGTCCAAGCCCTTCTGGGTGCTGGTCGTGGTTGGTGGCGTCCTCGCCTGCT |
| | | ACTCCCTCCTCGTCACGGTGGCGTTCATCATCTTCTGGGTGCGGAGCAAGCGAAGCCGCC |
| | | TCCTGCACAGCGACTACATGAACATGACGCCGCGCCGCCCGGGCCCGACCCGGAAGCAC |
| | | TACCAGCCCTACGCGCCGCCGCGCGACTTCGCGGCCTACCGCTCCCGGGTCAAGTTCTCC |
| | | CGCAGCGCCGACGCGCCCGCGTACCAGCAGGGCCAGAACCAGCTCTACAACGAGCTCAA |
| | | CCTGGGGAGGCGCGAGGAGTACGACGTGCTTGACAAGCGCCGTGGCCGGGACCCTGAGA |
| | | TGGGCGGTAAGCCGCGACGGAAGAACCCGCAGGAGGGCCTGTACAACGAGCTGCAGAA |
| | | GGACAAGATGGCGGAGGCCTACAGCGAGATCGGGATGAAGGGCGAGCGGCGGCGTGGC |
| | | AAGGGCCACGACGGCCTGTACCAGGGCCTCAGTACGGCCACCAAGGACACCTACGACGC |
| | | CTTGCACATGCAGGCCCTCCCGCCGCGCTAGTGATGA |
| 499 | Anti-CD19 CAR #39 nucleic | ATGGAGACCGACACCCTCCTGCTGTGGGTGCTGCTGCTTTGGGTGCCCGGGAGCACGGGC |
| | acid sequence | GAGGTGCAGCTGGTGGAGAGCGGCGGCGGCCTGGTCCAGGCCGGCGACAGCCTGAGGCT |
| | | CAGCTGCAGCCTGAGCGGCCGCACCTTCTCCAGCTACGCCATGGCCTGGTTCCGGCAGCT |
| | | GCCCGGGAAGGAGCGAGAGTTCGTGAGCGTGATCAGCCTGTCCGGCCAGAACACGTACT |
| | | ACGCCGACAGCGTCAAGGGCCGCTTCACCATCAGCCGGGACAACTCGAAGTCCACCGTG |
| | | TTCCTCCAGATGAACAGCCTGAAGCCCGAGGACACCGCCGTGTACTACTGCGCCGCGAG |
| | | GCCGGTCTACCACAAGTTCCACTACCGCGACTACGACTTCTGGGGCCAGGGCACCCTGGT |
| | | GACCGTGAGCTCCGCGAGCACCACCACGCCCGCGCCGCGGCCGCCGACGCCGGCGCCCA |
| | | CAATCGCCTCACAGCCACTGAGCCTGCGCCCGGAGGCCTGCAGGCCCGCCGCAGGCGGC |
| | | GCCGTGCACACGCGTGGGCTCGACTTCGCCTGTGACATATACATCTGGGCGCCCCTGGCC |
| | | GGCACCTGCGGCGTCCTGCTGCTGAGCCTGGTGATCACCCTGTATTGCAACCACCGGAAC |
| | | GTGCGCTCCAAACGCAGCCGGCTGCTGCACAGCGACTACATGAACATGACGCCGCGCAG |
| | | GCCCGGCCCGACCCGAAAGCACTACCAGCCATACGCGCCGCCGCGGGACTTCGCCGCCT |
| | | ACCGGTCAAGGGTTAAGTTCTCACGGTCGGCCGACGCGCCCGCCTACCAGCAGGGCCAG |
| | | AACCAGCTGTACAATGAGCTGAACCTGGGCCGGAGGGAGGAGTACGACGTACTGGATAA |
| | | GAGGCGCGGCAGAGACCCCGAGATGGGCGGCAAACCCAGGCGTAAGAACCCGCAGGAG |
| | | GGCCTGTACAACGAACTGCAGAAGGACAAGATGGCCGAGGCCTACAGCGAAATAGGCA |
| | | TGAAGGGCGAGCGTAGGCGCGGGAAGGGCCACGACGGCCTGTATCAAGGCCTGTCGACC |
| | | GCCACCAAAGACACCTACGACGCCCTGCACATGCAGGCCCTGCCGCCCAGGTAGTGATG |
| | | A |
| 500 | Anti-CD19 CAR #40 nucleic | ATGGAGACCGACACCCTCCTGCTGTGGGTGCTGCTGCTTTGGGTGCCCGGGAGCACGGGC |
| | acid sequence | GAGGTCCAGCTCGTGGAGAGCGGAGGCGGGCTCGTGCAGCCCGGCGGGAGCATGCGGCT |
| | | GTCGTGCCAGGGCTCCGGGCGCAGCTTCAGGCTGAATACCATGGTGTGGTACCGTCGAG |
| | | CGAGCGGCAACCAGAGGGAGCTGGTCGCCAGCCTGACCTCCGGCGGCAACACCTACTAC |
| | | GCCGACTCCGTGAAGGGCCGCTTCACAATCAGCCGGGACGACGCCAAGAGCACCGTCGA |
| | | CCTACAGATGAACAGCCTGAGGCCGGCCGACACCGCAGTGTACTTCTGCTACATGGGCG |
| | | GAAACAGCTGGGGCCAGGAAACGCAGGTGACCGTGAGCAGCGCGGCCGCGATAGAGGT |
| | | AATGTACCCTCCTCCTTACCTGGACAACGAGAAGTCGAACGGCACCATCATCCACGTCAA |
| | | GGGCAAGCACCTCTGCCCGTCGCCGCTGTTCCCGGGCCCGTCCAAGCCCTTCTGGGTGCT |
| | | GGTCGTGGTTGGTGGCGTCCTCGCCTGCTACTCCCTCCTCGTCACGGTGGCGTTCATCATC |
| | | TTCTGGGTGCGGAGCAAGCGAAGCCGCCTCCTGCACAGCGACTACATGAACATGACGCC |
| | | GCGCCGCCCGGGCCCGACCCGGAAGCACTACCAGCCCTACGCGCCGCCGCGCGACTTCG |
| | | CGGCCTACCGCTCCCGGGTCAAGTTCTCCCGCAGCGCCGACGCGCCCGCGTACCAGCAG |
| | | GGCCAGAACCAGCTCTACAACGAGCTCAACCTGGGGAGGCGCGAGGAGTACGACGTGCT |
| | | TGACAAGCGCCGTGGCCGGGACCCTGAGATGGGCGGTAAGCCGCGACGGAAGAACCCG |
| | | CAGGAGGGCCTGTACAACGAGCTGCAGAAGGACAAGATGGCGGAGGCCTACAGCGAGA |
| | | TCGGGATGAAGGGCGAGCGGCGGCGTGGCAAGGGCCACGACGGCCTGTACCAGGGCCTC |
| | | AGTACGGCCACCAAGGACACCTACGACGCCTTGCACATGCAGGCCCTCCCGCCGCGCTA |
| | | GTGATGA |
| 501 | Anti-CD19 CAR #41 nucleic | ATGGCCCTGCCCGTGACCGCCCTGCTGCTGCCACTGGCCCTGCTGCTGCACGCCGCCCGG |
| | acid sequence | CCCGAGGTGCAGCTGGTGGAGTCTGGCGGCGGAGTGGTGCAGCCCGGCGGCAGCCTGAG |
| | | ACTGAGCTGTGCCGCCTCCGGCCTATTTCTAAGCGACTACACGCTAGCCTGGTTTCGCCA |
| | | GGCCCCCGGCAAGGAGCGCGAGTTCGTGATCGTTTCCACGTGGAACCGCTATACCTACTA |
| | | TGCCGAAGGAGTGAAGGGCAGGTTCACCATCAGCAGAGACAACGCAAAAAAAACCGTG |
| | | TATCTGCAGATGAATAGCCTGAGGCCTGAGGATACCGCCCTGTACTACTGCGCCGCCAAA |
| | | TTCCGCATCTCATACAACGTTAGGGACGACAGCTTTTACGACCTGTGGGGCCAGGGCACC |
| | | CTGGTGACCGTGAGCTCCGCCGCCGCCATCGAGGTGATGTACCCCCCCCCTTACCTGGAC |
| | | AACGAGAAGTCCAACGGCACCATCATCCACGTGAAAGGCAAGCACCTGTGTCCCAGCCC |
| | | TCTGTTCCCCGGCCCAAGCAAGCCTTTCTGGGTGCTGGTGGTGGTGGGCGGCGTGCTGGC |
| | | CTGCTACTCCCTGCTGGTGACCGTGGCCTTCATCATCTTCTGGGTTAGAAGCAAGCGCTC |
| | | ACGCCTGCTACACTCAGACTACATGAACATGACGCCCAGAAGACCCGGACCTACCAGAA |
| | | AGCACTACCAACCATATGCCCCACCACGCGACTTTGCAGCATACAGAAGCCGGGTGAAA |
| | | TTCAGCCGGAGCGCCGACGCCCCCGCCTACCAGCAGGGCCAGAACCAGCTGTATAACGA |
| | | GCTGAACCTGGGCAGAAGGGAGGAGTACGACGTGCTGGACAAGCGCAGGGGCCGCGAC |
| | | CCTGAGATGGGCGGCAAGCCCAGGAGAAAGAACCCACAGGAGGGCCTGTATAACGAGC |
| | | TGCAGAAGGACAAGATGGCCGAGGCCTACAGCGAGATCGGCATGAAGGGAGAGCGCAG |
| | | GAGAGGCAAGGGCCACGACGGCCTGTACCAGGGCCTGTCCACCGCCACAAAGGACACAT |
| | | ACGACGCCCTGCACATGCAGGCCCTGCCCCCCAGGTAATAATAA |
| 502 | Anti-CD19 CAR #42 nucleic | ATGGCCCTGCCCGTGACCGCCCTGCTGCTGCCACTGGCCCTGCTGCTGCACGCCGCCCGG |
| | acid sequence | CCCGAGGTGCAGCTGGTGGAGTCTGGCGGCGGAGTGGTGCAGCCCGGCGGCAGCCTGAG |
| | | ACTGAGCTGTGCCGCCTCCGGCCTATTTCTAAGCGACTACACGCTAGCCTGGTTTCGCCA |
| | | GGCCCCCGGCAAGGAGCGCGAGTTCGTGATCGTTTCCACGTGGAACCGCTATACCTACTA |
| | | TGCCGAAGGAGTGAAGGGCAGGTTCACCATCAGCAGAGACAACGCAAAAAAAACCGTG |
| | | TATCTGCAGATGAATAGCCTGAGGCCTGAGGATACCGCCCTGTACTACTGCGCCGCCAAA |
| | | TTCCGCATCTCATACAACGTTAGGGACGACAGCTTTTACGACCTGTGGGGCCAGGGCACC |
| | | CTGGTGACCGTGAGCTCCGCCGCCGCCATCGAGGTGATGTACCCCCCCCCTTACCTGGAC |
| | | AACGAGAAGTCCAACGGCACCATCATCCACGTGAAAGGCAAGCACCTGTGTCCCAGCCC |
| | | TCTGTTCCCCGGCCCAAGCAAGCCTTTCTGGGTGCTGGTGGTGGTGGGCGGCGTGCTGGC |
| | | CTGCTACTCCCTGCTGGTGACCGTGGCCTTCATCATCTTCTGGAAGAGAGGACGCAAGAA |
| | | GCTGCTGTACATCTTCAAGCAACCATTCATGCGGCCCGTTCAAACGACCCAAGAAGAAG |
| | | ACGGCTGTAGCTGTAGGTTTCCAGAGGAAGAAGAGGGCGGCTGTGAACTACGGGTGAAA |
| | | TTCAGCCGGAGCGCCGACGCCCCCGCCTACCAGCAGGGCCAGAACCAGCTGTATAACGA |
| | | GCTGAACCTGGGCAGAAGGGAGGAGTACGACGTGCTGGACAAGCGCAGGGGCCGCGAC |
| | | CCTGAGATGGGCGGCAAGCCCAGGAGAAAGAACCCACAGGAGGGCCTGTATAACGAGC |
| | | TGCAGAAGGACAAGATGGCCGAGGCCTACAGCGAGATCGGCATGAAGGGAGAGCGCAG |
| | | GAGAGGCAAGGGCCACGACGGCCTGTACCAGGGCCTGTCCACCGCCACAAAGGACACAT |
| | | ACGACGCCCTGCACATGCAGGCCCTGCCCCCCAGGTAATAATAA |
| 503 | Anti-CD19 CAR #43 nucleic | ATGGCCCTGCCCGTGACCGCCCTGCTGCTGCCACTGGCCCTGCTGCTGCACGCCGCCCGG |
| | acid sequence | CCCGAGGTGCAGCTGGTGGAGTCTGGCGGCGGAGTGGTGCAGCCCGGCGGCAGCCTGAG |
| | | ACTGAGCTGTGCCGCCTCCGGCGACACCTTCGGAACGAAAGCCATGGGATGGTTTCGCC |
| | | AGGCCCCCGGCAAGGGACGCGAGTTCGTGGCCGCAATCACGTGGACCGCAGGCCTAACC |
| | | TACTATGCCGACAGCGTGAAGGGCAGGTTCACCATCAGCAAAGACCGCGCAAAAAACAC |
| | | CGTGGACCTGCAGATGAATAGCCTGAGGCCTGAGGATACCGCCCTGTACTACTGCGCCTC |
| | | ACGGCGCACGTTTTCATACGAGCACCCTAACTACTATACCAGGTCAGCCTACCACTTTTG |
| | | GGGCCAGGGCACCCTGGTGACCGTGAGCTCCGCCGCCGCCATCGAGGTGATGTACCCCC |
| | | CCCCTTACCTGGACAACGAGAAGTCCAACGGCACCATCATCCACGTGAAAGGCAAGCAC |
| | | CTGTGTCCCAGCCCTCTGTTCCCCGGCCCAAGCAAGCCTTTCTGGGTGCTGGTGGTGGTG |
| | | GGCGGCGTGCTGGCCTGCTACTCCCTGCTGGTGACCGTGGCCTTCATCATCTTCTGGGTTA |
| | | GAAGCAAGCGCTCACGCCTGCTACACTCAGACTACATGAACATGACGCCCAGAAGACCC |
| | | GGACCTACCAGAAAGCACTACCAACCATATGCCCCACCACGCGACTTTGCAGCATACAG |
| | | AAGCCGGGTGAAATTCAGCCGGAGCGCCGACGCCCCCGCCTACCAGCAGGGCCAGAACC |
| | | AGCTGTATAACGAGCTGAACCTGGGCAGAAGGGAGGAGTACGACGTGCTGGACAAGCG |
| | | CAGGGGCCGCGACCCTGAGATGGGCGGCAAGCCCAGGAGAAAGAACCCACAGGAGGGC |
| | | CTGTATAACGAGCTGCAGAAGGACAAGATGGCCGAGGCCTACAGCGAGATCGGCATGAA |
| | | GGGAGAGCGCAGGAGAGGCAAGGGCCACGACGGCCTGTACCAGGGCCTGTCCACCGCC |
| | | ACAAAGGACACATACGACGCCCTGCACATGCAGGCCCTGCCCCCCAGGTAATAATAA |
| 504 | Anti-CD19 CAR #44 nucleic | ATGGCCCTGCCCGTGACCGCCCTGCTGCTGCCACTGGCCCTGCTGCTGCACGCCGCCCGG |
| | acid sequence | CCCGAGGTGCAGCTGGTGGAGTCTGGCGGCGGAGTGGTGCAGCCCGGCGGCAGCCTGAG |
| | | ACTGAGCTGTGCCGCCTCCGGCGACACCTTCGGAACGAAAGCCATGGGATGGTTTCGCC |
| | | AGGCCCCCGGCAAGGGACGCGAGTTCGTGGCCGCAATCACGTGGACCGCAGGCCTAACC |
| | | TACTATGCCGACAGCGTGAAGGGCAGGTTCACCATCAGCAAAGACCGCGCAAAAAACAC |
| | | CGTGGACCTGCAGATGAATAGCCTGAGGCCTGAGGATACCGCCCTGTACTACTGCGCCTC |
| | | ACGGCGCACGTTTTCATACGAGCACCCTAACTACTATACCAGGTCAGCCTACCACTTTTG |
| | | GGGCCAGGGCACCCTGGTGACCGTGAGCTCCGCCGCCGCCATCGAGGTGATGTACCCCC |
| | | CCCCTTACCTGGACAACGAGAAGTCCAACGGCACCATCATCCACGTGAAAGGCAAGCAC |
| | | CTGTGTCCCAGCCCTCTGTTCCCCGGCCCAAGCAAGCCTTTCTGGGTGCTGGTGGTGGTG |
| | | GGCGGCGTGCTGGCCTGCTACTCCCTGCTGGTGACCGTGGCCTTCATCATCTTCTGGAAG |
| | | AGAGGACGCAAGAAGCTGCTGTACATCTTCAAGCAACCATTCATGCGGCCCGTTCAAAC |
| | | GACCCAAGAAGAAGACGGCTGTAGCTGTAGGTTTCCAGAGGAAGAAGAGGGCGGCTGT |
| | | GAACTACGGGTGAAATTCAGCCGGAGCGCCGACGCCCCCGCCTACCAGCAGGGCCAGAA |
| | | CCAGCTGTATAACGAGCTGAACCTGGGCAGAAGGGAGGAGTACGACGTGCTGGACAAGC |
| | | GCAGGGGCCGCGACCCTGAGATGGGCGGCAAGCCCAGGAGAAAGAACCCACAGGAGGG |
| | | CCTGTATAACGAGCTGCAGAAGGACAAGATGGCCGAGGCCTACAGCGAGATCGGCATGA |
| | | AGGGAGAGCGCAGGAGAGGCAAGGGCCACGACGGCCTGTACCAGGGCCTGTCCACCGC |
| | | CACAAAGGACACATACGACGCCCTGCACATGCAGGCCCTGCCCCCCAGGTAATAATAA |
| 505 | Anti-CD19 CAR #45 nucleic | ATGGCCCTGCCCGTGACCGCCCTGCTGCTGCCACTGGCCCTGCTGCTGCACGCCGCCCGG |
| | acid sequence | CCCGAGGTGCAGCTGGTGGAGTCTGGCGGCGGAGTGGTGCAGCCCGGCGGCAGCCTGAG |
| | | ACTGAGCTGTGCCGCCTCCGGCTTTACCTTCAGCAGCTACGCCATGGCCTGGTTTCGCCA |
| | | GGCCCCCGGCAAGGAGCGCGAGTTCGTGGCCGTGATCTCCCTATCAGGCGGCAGCGTTG |
| | | ACTATCGCGACAGCGTGAAGGGCAGGTTCACCATCAGCAGAGACAACGCAAAAAACACC |
| | | GTGGACCTGCAGATGAATAGCCTGAGGCCTGAGGATACCGCCCTGTACTACTGCGCCGC |
| | | CCGGCCTGTTTATATCAAATGGTCCGAAAGGGACTTTGACTATTGGGGCCAGGGCACCCT |
| | | GGTGACCGTGAGCTCCGCCGCCGCCATCGAGGTGATGTACCCCCCCCCTTACCTGGACAA |
| | | CGAGAAGTCCAACGGCACCATCATCCACGTGAAAGGCAAGCACCTGTGTCCCAGCCCTC |
| | | TGTTCCCCGGCCCAAGCAAGCCTTTCTGGGTGCTGGTGGTGGTGGGCGGCGTGCTGGCCT |
| | | GCTACTCCCTGCTGGTGACCGTGGCCTTCATCATCTTCTGGGTTAGAAGCAAGCGCTCAC |
| | | GCCTGCTACACTCAGACTACATGAACATGACGCCCAGAAGACCCGGACCTACCAGAAAG |
| | | CACTACCAACCATATGCCCCACCACGCGACTTTGCAGCATACAGAAGCCGGGTGAAATT |
| | | CAGCCGGAGCGCCGACGCCCCCGCCTACCAGCAGGGCCAGAACCAGCTGTATAACGAGC |
| | | TGAACCTGGGCAGAAGGGAGGAGTACGACGTGCTGGACAAGCGCAGGGGCCGCGACCC |
| | | TGAGATGGGCGGCAAGCCCAGGAGAAAGAACCCACAGGAGGGCCTGTATAACGAGCTG |
| | | CAGAAGGACAAGATGGCCGAGGCCTACAGCGAGATCGGCATGAAGGGAGAGCGCAGGA |
| | | GAGGCAAGGGCCACGACGGCCTGTACCAGGGCCTGTCCACCGCCACAAAGGACACATAC |
| | | GACGCCCTGCACATGCAGGCCCTGCCCCCCAGGTAATAATAA |
| 506 | Anti-CD19 CAR #46 nucleic | ATGGCCCTGCCCGTGACCGCCCTGCTGCTGCCACTGGCCCTGCTGCTGCACGCCGCCCGG |
| | acid sequence | CCCGAGGTGCAGCTGGTGGAGTCTGGCGGCGGAGTGGTGCAGCCCGGCGGCAGCCTGAG |
| | | ACTGAGCTGTGCCGCCTCCGGCTTTACCTTCAGCAGCTACGCCATGGCCTGGTTTCGCCA |
| | | GGCCCCCGGCAAGGAGCGCGAGTTCGTGGCCGTGATCTCCCTATCAGGCGGCAGCGTTG |
| | | ACTATCGCGACAGCGTGAAGGGCAGGTTCACCATCAGCAGAGACAACGCAAAAAACACC |
| | | GTGGACCTGCAGATGAATAGCCTGAGGCCTGAGGATACCGCCCTGTACTACTGCGCCGC |
| | | CCGGCCTGTTTATATCAAATGGTCCGAAAGGGACTTTGACTATTGGGGCCAGGGCACCCT |
| | | GGTGACCGTGAGCTCCGCCGCCGCCATCGAGGTGATGTACCCCCCCCCTTACCTGGACAA |
| | | CGAGAAGTCCAACGGCACCATCATCCACGTGAAAGGCAAGCACCTGTGTCCCAGCCCTC |
| | | TGTTCCCCGGCCCAAGCAAGCCTTTCTGGGTGCTGGTGGTGGTGGGCGGCGTGCTGGCCT |
| | | GCTACTCCCTGCTGGTGACCGTGGCCTTCATCATCTTCTGGAAGAGAGGACGCAAGAAGC |
| | | TGCTGTACATCTTCAAGCAACCATTCATGCGGCCCGTTCAAACGACCCAAGAAGAAGAC |
| | | GGCTGTAGCTGTAGGTTTCCAGAGGAAGAAGAGGGCGGCTGTGAACTACGGGTGAAATT |
| | | CAGCCGGAGCGCCGACGCCCCCGCCTACCAGCAGGGCCAGAACCAGCTGTATAACGAGC |
| | | TGAACCTGGGCAGAAGGGAGGAGTACGACGTGCTGGACAAGCGCAGGGGCCGCGACCC |
| | | TGAGATGGGCGGCAAGCCCAGGAGAAAGAACCCACAGGAGGGCCTGTATAACGAGCTG |
| | | CAGAAGGACAAGATGGCCGAGGCCTACAGCGAGATCGGCATGAAGGGAGAGCGCAGGA |
| | | GAGGCAAGGGCCACGACGGCCTGTACCAGGGCCTGTCCACCGCCACAAAGGACACATAC |
| | | GACGCCCTGCACATGCAGGCCCTGCCCCCCAGGTAATAATAA |
| 507 | Anti-CD19 CAR #47 nucleic | ATGGCTCTGCCCGTGACCGCACTGCTGCTGCCCCTGGCCCTGCTGCTGCACGCCGCCAGG |
| | acid sequence | CCCGAGGTGCAGCTGGTGGAGTCCGGCGGCGGCGTGGTGCAGCCAGGCGGCTCTCTGAG |
| | | GCTGAGCTGCGCCGCCAGCGGCGACACCTTCGGAACGAAAGCCATGGGATGGTTCAGGC |
| | | AGGCCCCCGGCAAGGGACGCGAGTTTGTGGCCGCAATCACGTGGACAGCAGGCCTAACC |
| | | TACTACGCTGACTCCGTGAAGGGCAGGTTCACAATTTCTAAAGACCGCGCCAAGAACAC |
| | | CGTGGATCTGCAGATGAACTCCCTGCGCCCCGAGGACACAGCCCTGTACTACTGCGCCTC |
| | | ACGGCGCACGTTCTCATACGAGCACCCCAATTACTATACCAGGTCAGCCTACCACTTTTG |
| | | GGGACAGGGCACCCTGGTGACCGTGTCCTCCGCCGCCGCTATCGAGGTGATGTACCCTCC |
| | | CCCTTACCTGGACAACGAGAAATCAAACGGCACCATCATCCACGTGAAGGGAAAGCACC |
| | | TGTGTCCTAGCCCACTGTTCCCAGGCCCCAGCAAGCCCTTTTGGGTGCTGGTTGTGGTGG |
| | | GCGGCGTGCTGGCCTGCTACAGCCTGCTGGTCACCGTGGCCTTCATCATCTTCTGGGTTC |
| | | GGTCCAAGCGCTCACGCCTGCTACACTCAGACTACATGAACATGACGCCCCGCAGACCT |
| | | GGACCTACCAGGAAACACTATCAACCATATGCCCCCCCAAGGGACTTTGCAGCATACAG |
| | | GAGCCGCGTGAAGTTCTCCAGGAGCGCCGATGCCCCCGCCTACCAGCAGGGCCAGAACC |
| | | AGCTGTACAACGAGCTGAACCTGGGCCGGCGGGAGGAATACGACGTGCTGGACAAGCG |
| | | GAGAGGCCGGGACCCTGAGATGGGCGGAAAGCCTAGACGCAAGAACCCCCAGGAGGGC |
| | | CTGTACAACGAGCTGCAGAAAGATAAAATGGCCGAGGCCTATAGCGAGATCGGCATGAA |
| | | GGGCGAGAGAAGACGCGGCAAGGGGCACGATGGCCTGTACCAGGGGCTGAGCACCGCC |
| | | ACCAAAGACACCTACGATGCCCTGCACATGCAGGCCCTGCCCCCGCGGTAATAATAA |
| 508 | Anti-CD19 CAR #48 nucleic | ATGGCTCTGCCCGTGACCGCACTGCTGCTGCCCCTGGCCCTGCTGCTGCACGCCGCCAGG |
| | acid sequence | CCCGAGGTGCAGCTGGTGGAGTCCGGCGGCGGCGTGGTGCAGCCAGGCGGCTCTCTGAG |
| | | GCTGAGCTGCGCCGCCAGCGGCTTTACCTTCAGCTCCTACGCCATGGCCTGGTTCAGGCA |
| | | GGCCCCCGGCAAGGAGCGCGAGTTTGTGGCCGTGATCAGCCTATCAGGCGGCAGCGTTG |
| | | ACTACCGCGACTCCGTGAAGGGCAGGTTCACAATTTCTCGGGACAACGCCAAGAACACC |
| | | GTGGATCTGCAGATGAACTCCCTGCGCCCCGAGGACACAGCCCTGTACTACTGCGCCGCA |
| | | CGGCCCGTTTATATCAAATGGAGCGAAAGGGACTTTGACTATTGGGGACAGGGCACCCT |
| | | GGTGACCGTGTCCTCCGCCGCCGCTATCGAGGTGATGTACCCTCCCCCTTACCTGGACAA |
| | | CGAGAAATCAAACGGCACCATCATCCACGTGAAGGGAAAGCACCTGTGTCCTAGCCCAC |
| | | TGTTCCCAGGCCCCAGCAAGCCCTTTTGGGTGCTGGTTGTGGTGGGCGGCGTGCTGGCCT |
| | | GCTACAGCCTGCTGGTCACCGTGGCCTTCATCATCTTCTGGGTTCGGTCCAAGCGCTCAC |
| | | GCCTGCTACACTCAGACTACATGAACATGACGCCCCGCAGACCTGGACCTACCAGGAAA |
| | | CACTATCAACCATATGCCCCCCCAAGGGACTTTGCAGCATACAGGAGCCGCGTGAAGTTC |
| | | TCCAGGAGCGCCGATGCCCCCGCCTACCAGCAGGGCCAGAACCAGCTGTACAACGAGCT |
| | | GAACCTGGGCCGGCGGGAGGAATACGACGTGCTGGACAAGCGGAGAGGCCGGGACCCT |
| | | GAGATGGGCGGAAAGCCTAGACGCAAGAACCCCCAGGAGGGCCTGTACAACGAGCTGC |
| | | AGAAAGATAAAATGGCCGAGGCCTATAGCGAGATCGGCATGAAGGGCGAGAGAAGACG |
| | | CGGCAAGGGGCACGATGGCCTGTACCAGGGGCTGAGCACCGCCACCAAAGACACCTACG |
| | | ATGCCCTGCACATGCAGGCCCTGCCCCCGCGGTAATAATAA |
| 509 | Anti-CD19 CAR #49 nucleic | ATGGCTCTGCCCGTGACCGCACTGCTGCTGCCCCTGGCCCTGCTGCTGCACGCCGCCAGG |
| | acid sequence | CCCGAGGTGCAGCTGGTGGAGTCCGGCGGCGGCGTGGTGCAGCCAGGCGGCTCTCTGAG |
| | | GCTGAGCTGCGCCGCCAGCGGCTTTACCTTCAGCTCCTACGCCATGGCCTGGTTCAGGCA |
| | | GGCCCCCGGCAAGGAGCGCGAGTTTGTGGCCGTGATCAGCCTATCAGGCGGCAGCGTTG |
| | | ACTACCGCGACTCCGTGAAGGGCAGGTTCACAATTTCTCGGGACAACGCCAAGAACACC |
| | | GTGGATCTGCAGATGAACTCCCTGCGCCCCGAGGACACAGCCCTGTACTACTGCGCCGCA |
| | | CGGCCCGTTTATATCAAATGGAGCGAAAGGGACTTTGACTATTGGGGACAGGGCACCCT |
| | | GGTGACCGTGTCCTCCGCCGCCGCTATCGAGGTGATGTACCCTCCCCCTTACCTGGACAA |
| | | CGAGAAATCAAACGGCACCATCATCCACGTGAAGGGAAAGCACCTGTGTCCTAGCCCAC |
| | | TGTTCCCAGGCCCCAGCAAGCCCTTTTGGGTGCTGGTTGTGGTGGGCGGCGTGCTGGCCT |
| | | GCTACAGCCTGCTGGTCACCGTGGCCTTCATCATCTTCTGGAAGCGGGGACGCAAGAAGC |
| | | TGCTGTATATCTTTAAGCAACCATTCATGCGGCCCGTTCAAACGACTCAAGAAGAAGACG |
| | | GGTGTAGCTGCCGGTTTCCCGAAGAAGAAGAGGGCGGCTGTGAACTACGCGTGAAGTTC |
| | | TCCAGGAGCGCCGATGCCCCCGCCTACCAGCAGGGCCAGAACCAGCTGTACAACGAGCT |
| | | GAACCTGGGCCGGCGGGAGGAATACGACGTGCTGGACAAGCGGAGAGGCCGGGACCCT |
| | | GAGATGGGCGGAAAGCCTAGACGCAAGAACCCCCAGGAGGGCCTGTACAACGAGCTGC |
| | | AGAAAGATAAAATGGCCGAGGCCTATAGCGAGATCGGCATGAAGGGCGAGAGAAGACG |
| | | CGGCAAGGGGCACGATGGCCTGTACCAGGGGCTGAGCACCGCCACCAAAGACACCTACG |
| | | ATGCCCTGCACATGCAGGCCCTGCCCCCGCGGTAATAATAA |
| 510 | Anti-CD19 CAR #50 nucleic | ATGTTGCTGCTCGTGACATCTCTGCTCCTCTGTGAGCTCCCCCATCCAGCATTTCTGCTCA |
| | acid sequence | TCCCCGACATCCAGATGACACAGACAACCAGCAGCCTGAGCGCCAGCCTGGGCGACAGA |
| | | GTGACCATCTCCTGCCGCGCCAGCCAGGATATCTCCAAGTACCTGAACTGGTACCAGCAG |
| | | AAGCCCGACGGCACAGTGAAGCTGCTGATCTACCACACCAGCAGGCTGCACTCCGGCGT |
| | | GCCCTCCAGGTTCAGCGGCTCTGGCAGCGGGACCGATTACAGCCTGACCATCAGCAATCT |
| | | GGAGCAGGAGGACATCGCCACCTACTTCTGCCAGCAGGGCAACACCCTGCCCTACACAT |
| | | TCGGCGGCGGCACCAAGCTGGAGATCACCGGCTCCACCTCCGGCTCTGGCAAGCCCGGC |
| | | AGCGGCGAGGGCTCCACCAAGGGCGAGGTGAAGCTGCAGGAGAGCGGCCCCGGCCTGG |
| | | TGGCCCCCAGCCAGAGTCTGTCCGTGACCTGCACAGTGAGCGGCGTGTCCCTGCCAGACT |
| | | ATGGCGTGAGCTGGATCAGGCAGCCACCCAGGAAGGGCCTGGAGTGGCTGGGCGTGATC |
| | | TGGGGAAGCGAGACTACCTATTACAACTCCGCCCTGAAGTCCAGGCTGACCATCATCAA |
| | | GGACAACAGCAAGTCCCAGGTGTTCCTGAAGATGAACTCCCTGCAGACCGATGACACCG |
| | | CCATCTACTACTGCGCCAAACACTACTACTACGGCGGCAGCTACGCCATGGACTACTGGG |
| | | GCCAGGGCACCAGCGTGACCGTGAGCAGCGCCGCCGCCATCGAGGTGATGTACCCACCC |
| | | CCATACCTGGATAACGAGAAGTCCAATGGCACCATCATCCACGTGAAGGGCAAGCACCT |
| | | GTGTCCCTCCCCCCTGTTCCCCGGCCCCAGCAAGCCCTTCTGGGTGCTGGTGGTGGTGGG |
| | | CGGCGTGCTGGCCTGCTACAGCCTGCTGGTGACAGTGGCCTTCATCATCTTCTGGGTGCG |
| | | CAGCAAGAGGTCTAGGCTGCTGCACAGCGACTACATGAATATGACCCCCCGGCGGCCCG |
| | | GCCCCACCAGAAAGCACTATCAGCCCTACGCCCCCCCAAGAGACTTCGCCGCCTACAGA |
| | | AGCCGGGTGAAGTTCAGTAGAAGCGCCGACGCCCCCGCCTACCAGCAGGGCCAGAACCA |
| | | GCTGTACAATGAGCTGAATCTGGGCCGCCGCGAGGAGTACGACGTGCTGGATAAGAGAA |
| | | GAGGCAGGGACCCCGAGATGGGCGGCAAGCCTAGGAGAAAGAACCCTCAGGAGGGGCT |
| | | GTACAACGAGCTGCAGAAGGATAAGATGGCCGAGGCCTACAGCGAGATCGGCATGAAG |
| | | GGGGAGAGAAGGAGAGGCAAGGGCCACGACGGCCTGTACCAGGGCCTGAGCACCGCTA |
| | | CCAAGGACACCTACGACGCCCTGCACATGCAGGCCCTGCCCCCCAGATAATAATAA |
| 511 | Anti-CD19 CAR #51 nucleic | ATGTTGCTGTTGGTCACCAGCTTGCTGTTATGTGAATTGCCTCACCCGGCCTTCCTCTTGA |
| | acid sequence | TCCCCGACATCCAGATGACCCAGACCACCTCTTCCCTGTCCGCCTCCCTGGGCGATAGGG |
| | | TGACCATCAGCTGCAGGGCCTCCCAGGACATCAGCAAGTACCTGAATTGGTACCAGCAG |
| | | AAGCCCGATGGAACCGTGAAGCTGCTGATCTACCACACCTCCCGCCTGCACAGCGGCGT |
| | | GCCCAGCAGGTTCTCCGGCAGCGGCTCCGGAACCGATTACAGCCTGACCATCAGCAACC |
| | | TGGAGCAGGAGGACATCGCCACCTACTTTTGCCAGCAGGGGAACACCCTGCCCTACACA |
| | | TTCGGCGGCGGCACCAAGCTGGAGATCACCGGCTCCACCTCCGGCTCTGGCAAGCCCGG |
| | | CAGCGGCGAGGGCTCCACCAAGGGCGAGGTGAAGCTGCAGGAGAGCGGCCCCGGCCTG |
| | | GTGGCCCCCAGCCAGAGTCTGTCCGTGACCTGCACAGTGAGCGGCGTGTCCCTGCCAGAC |
| | | TATGGCGTGAGCTGGATCAGGCAGCCACCCAGGAAGGGCCTGGAGTGGCTGGGCGTGAT |
| | | CTGGGGAAGCGAGACTACCTATTACAACTCCGCCCTGAAGTCCAGACTGACCATCATCA |
| | | AGGATAACAGCAAGAGCCAGGTGTTCCTGAAGATGAACAGCCTGCAGACCGACGACACC |
| | | GCCATCTACTACTGCGCCAAGCACTACTACTACGGCGGCTCCTACGCCATGGACTACTGG |
| | | GGCCAGGGCACAAGCGTGACTGTGTCTAGCGCCGCCGCAATCGAGGTGATGTACCCCCC |
| | | TCCCTACCTGGACAACGAGAAAAGCAACGGCACCATTATCCATGTCAAAGGCAAGCACC |
| | | TGTGCCCTTCCCCCCTGTTCCCCGGCCCCAGCAAGCCCTTCTGGGTGCTGGTGGTGGTGG |
| | | GCGGCGTGCTGGCCTGTTACAGCCTGCTGGTGACCGTGGCCTTCATCATCTTCTGGGTGA |
| | | GGAGCAAGCGGTCCAGACTGCTGCACAGCGACTACATGAACATGACACCAAGAAGACCA |
| | | GGCCCTACCAGAAAGCACTACCAGCCCTATGCCCCTCCCAGGGACTTCGCCGCCTATAGA |
| | | TCTCGGGTGAAGTTCTCAAGAAGCGCCGACGCCCCTGCCTACCAGCAGGGCCAGAACCA |
| | | GCTGTATAACGAACTGAACCTGGGCCGGAGGGAGGAGTACGATGTGCTGGACAAGAGA |
| | | AGGGGCAGGGACCCAGAGATGGGGGGAAAGCCTCGGAGGAAGAATCCCCAGGAGGGCC |
| | | TGTATAACGAGCTGCAGAAGGACAAGATGGCCGAGGCCTACAGCGAGATTGGCATGAAG |
| | | GGCGAGAGACGGCGCGGCAAGGGGCACGACGGCCTGTACCAGGGCCTGTCTACCGCCAC |
| | | AAAGGACACATACGACGCCCTGCATATGCAGGCCCTGCCACCCAGGTAATAATAA |
| 512 | | |
| 514 | O49 (Pro L) light chain | EIVLTQTPSSLSASLGDRVTISCRASQSIGSSLHWYQQKPDQSPKLLIKYASQSFSGVPSKFIGS |
| | | GSGTDFTLTINSLEAEDAATFYCHQSSTLPYTFGQGTKLEIK |
| 515 | CD8α Signal peptide | MALPVTALLLPLALLLHAARP |
| 516 | 5′UTR (MRT14577) | GGACAGATCGCCTGGAGACGCCATCCACGCTGTTTTGACCTCCATAGAAGACACCGGGA |
| | | CCGATCCAGCCTCCGCGGCCGGGAACGGTGCATTGGAACGCGGATTCCCCGTGCCAAGA |
| | | GTGACTCACCGTCCTTGACACG |
| 517 | 3′URT (MRT14577) | CGGGTGGCATCCCTGTGACCCCTCCCCAGTGCCTCTCCTGGCCCTGGAAGTIGCCACTCC |
| | | AGTGCCCACCAGCCTTGTCCTAATAAAATTAAGTTGCATC |
| 518 | 5′UTR (TTR-102) | GGGTCAAGATTAGAGAACGGTCGTAGCATTATCGGAGGTTCTGCCACC |
| 519 | 3′UTR (TTR-102) | GGCACATTTGAATAAATTCTATTACCAGTTCAAGCTTGGCGTAATCATGGTCATAGCTG |
| 520 | Igκ signal peptide | MDMRVPAQLLGLLLLWLPGAKC |
| 321 | CD8alpha hinge- | LSNSIMYFSHFVPVFLPAKPTTTPAPRPPTPAPTIASQPLSLRPEACRPAAGGAVHTRGLDFAC |
| | transmembrane (66) | DIYIWAPLAGTCGVLLLSLVITLYCNHRN |
| 522 | CD28 hinge-transmembrane | IEVMYPPPYLDNERSNGTIIHVKGKHLCPSPLFPGPSKPFWVLVVVGGVLACYSLLVTVAFIIF |
| | domain | WV |
| 538 | 46 aa CD8 hinge- | TTTPAPRPPTPAPTIASQPLSLRPEACRPAAGGAVHTRGLDFACDIYIWAPLAGTCGVLLLSLV |
| | transmembrane | ITLYCNHRN |
| 339 | 26 aa CD8 hinge- | SLRPEACRPAAGGAVHTRGLDFACDIYIWAPLAGTCGVLLLSLVITLYCNARN |
| | transmembrane | |
| 540 | 66 aa CD8 hinge- | LSNSIMYFSHFVPVFLPAKPTTTPAPRPPTPAPTIASQPLSLRPEACRPAAGGAVHTRGLDFAC |
| | transmembrane | DIYIWAPLAGTCGVLLLSLVITLYCNHRN |
| 541 | CD28 Hinge #3 | IEVMYPPPYLDNEKSNGTIIHVKGKHLCPSPLFPGPSKPF |
| 542 | CD28 Transmembrane | WVLVVVGGVLACYSLLVTVAFIIFWV |
| | domain #2 | |
| 543 | CD28 co-stim domain | RSKRSRLLHSDYMNMTPRRPGPTRKHYQPYAPPRDFAAYRS |
| 544 | CD3 zeta intracellular domain | RVKFSRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 565 | Human CD8 alpha chain | malpvtalll plalllhaar psqfrvspld rtwnlgetve lkcqvllsnp tsgcswlfqp |
| | isoform 1 precursor | rgaaasptfl lylsqnkpka aegldtqrfs gkrlgdtfvl tlsdfrrene gyyfcsalsn |
| | | simyfshfvp vflpakpttt paprpptpap tiasqplslr peacrpaagg avhtrgldfa |
| | | cdiyiwapla gtcgvlllsl vitlycnhrn rrrvckeprp vvksgdkpsl saryv |
| 566 | Albumin VHH CD1 | GFTFRSFGMS |
| 567 | Albumin VHH CD2 | SISGSGSDTL |
| 568 | Albumin VHH CD3 | GGSLSR |
| 569 | frame sequence of VHH1 type | QVQLVESGGGLVQPGGSLRLSCAASGFTLDYYAIGWFRQAPGKEREGVSCISSSDGSTYYAD |
| | ISVD | SVKGRFTISRDNAKNTVYLQMNSLKPEDTAVYYCAA |
| 570 | Anti-CD19 CAR #1 mRNA | AUGGCUCUGCCCGUGACCGCACUGCUGCUGCCCCUGGCCCUGCUGCUGCACGCCGCCA |
| | sequence (full open reading | GGCCCGAGGUGCAGCUGGUGGAGUCCGGCGGCGGCGUGGUGCAGCCAGGCGGCUCUCU |
| | frame) | GAGGCUGAGCUGCGCCGCCAGCGGCCUAUUUCUAAGCGACUACACGCUAGCCUGGUUC |
| | | AGGCAGGCCCCCGGCAAGGAGCGCGAGUUUGUGAUCGUUAGCACGUGGAACCGCUAU |
| | | ACCUACUACGCUGAAGGAGUGAAGGGCAGGUUCACAAUUUCUCGGGACAACGCCAAG |
| | | AAAACCGUGUAUCUGCAGAUGAACUCCCUGCGCCCCGAGGACACAGCCCUGUACUACU |
| | | GCGCCGCAAAAUUUCGCAUCUCAUACAACGUUAGGGACGACAGCUUCUACGACCUGUG |
| | | GGGACAGGGCACCCUGGUGACCGUGUCCUCCGCCGCCGCUAUCGAGGUGAUGUACCCU |
| | | CCCCCUUACCUGGACAACGAGAAAUCAAACGGCACCAUCAUCCACGUGAAGGGAAAGC |
| | | ACCUGUGUCCUAGCCCACUGUUCCCAGGCCCCAGCAAGCCCUUUUGGGUGCUGGUUGU |
| | | GGUGGGCGGCGUGCUGGCCUGCUACAGCCUGCUGGUCACCGUGGCCUUCAUCAUCUUC |
| | | UGGGUUCGGUCCAAGCGCUCACGCCUGCUACACUCAGACUACAUGAACAUGACGCCCC |
| | | GCAGACCUGGACCUACCAGGAAACACUAUCAACCAUAUGCCCCCCCAAGGGACUUUGC |
| | | AGCAUACAGGAGCCGCGUGAAGUUCUCCAGGAGCGCCGAUGCCCCCGCCUACCAGCAG |
| | | GGCCAGAACCAGCUGUACAACGAGCUGAACCUGGGCCGGCGGGAGGAAUACGACGUGC |
| | | UGGACAAGCGGAGAGGCCGGGACCCUGAGAUGGGCGGAAAGCCUAGACGCAAGAACC |
| | | CCCAGGAGGGCCUGUACAACGAGCUGCAGAAAGAUAAAAUGGCCGAGGCCUAUAGCG |
| | | AGAUCGGCAUGAAGGGCGAGAGAAGACGCGGCAAGGGGCACGAUGGCCUGUACCAGG |
| | | GGCUGAGCACCGCCACCAAAGACACCUACGAUGCCCUGCACAUGCAGGCCCUGCCCCC |
| | | GCGGUAAUAAUAA |
| 571 | Anti-CD19 CAR #2 mRNA | AUGGCUCUGCCCGUGACCGCACUGCUGCUGCCCCUGGCCCUGCUGCUGCACGCCGCCA |
| | sequence (full open reading | GGCCCGAGGUGCAGCUGGUGGAGUCCGGCGGCGGCGUGGUGCAGCCAGGCGGCUCUCU |
| | frame) | GAGGCUGAGCUGCGCCGCCAGCGGCCUAUUUCUAAGCGACUACACGCUAGCCUGGUUC |
| | | AGGCAGGCCCCCGGCAAGGAGCGCGAGUUUGUGAUCGUUAGCACGUGGAACCGCUAU |
| | | ACCUACUACGCUGAAGGAGUGAAGGGCAGGUUCACAAUUUCUCGGGACAACGCCAAG |
| | | AAAACCGUGUAUCUGCAGAUGAACUCCCUGCGCCCCGAGGACACAGCCCUGUACUACU |
| | | GCGCCGCAAAAUUUCGCAUCUCAUACAACGUUAGGGACGACAGCUUCUACGACCUGUG |
| | | GGGACAGGGCACCCUGGUGACCGUGUCCUCCGCCGCCGCUAUCGAGGUGAUGUACCCU |
| | | CCCCCUUACCUGGACAACGAGAAAUCAAACGGCACCAUCAUCCACGUGAAGGGAAAGC |
| | | ACCUGUGUCCUAGCCCACUGUUCCCAGGCCCCAGCAAGCCCUUUUGGGUGCUGGUUGU |
| | | GGUGGGCGGCGUGCUGGCCUGCUACAGCCUGCUGGUCACCGUGGCCUUCAUCAUCUUC |
| | | UGGAAGCGGGGACGCAAGAAGCUGCUGUAUAUCUUUAAGCAACCAUUCAUGCGGCCC |
| | | GUUCAAACGACUCAAGAAGAAGACGGGUGUAGCUGCCGGUUUCCCGAAGAAGAAGAG |
| | | GGCGGCUGUGAACUACGCGUGAAGUUCUCCAGGAGCGCCGAUGCCCCCGCCUACCAGC |
| | | AGGGCCAGAACCAGCUGUACAACGAGCUGAACCUGGGCCGGCGGGAGGAAUACGACG |
| | | UGCUGGACAAGCGGAGAGGCCGGGACCCUGAGAUGGGCGGAAAGCCUAGACGCAAGA |
| | | ACCCCCAGGAGGGCCUGUACAACGAGCUGCAGAAAGAUAAAAUGGCCGAGGCCUAUA |
| | | GCGAGAUCGGCAUGAAGGGCGAGAGAAGACGCGGCAAGGGGCACGAUGGCCUGUACC |
| | | AGGGGCUGAGCACCGCCACCAAAGACACCUACGAUGCCCUGCACAUGCAGGCCCUGCC |
| | | CCCGCGGUAAUAAUAA |
| 572 | Anti-CD19 CAR #3 mRNA | AUGGCUCUGCCCGUGACCGCACUGCUGCUGCCCCUGGCCCUGCUGCUGCACGCCGCCA |
| | sequence (full open reading | GGCCCGAGGUGCAGCUGGUGGAGUCCGGCGGCGGCGUGGUGCAGCCAGGCGGCUCUCU |
| | frame) | GAGGCUGAGCUGCGCCGCCAGCGGCGACACCUUCGGAACGAAAGCCAUGGGAUGGUUC |
| | | AGGCAGGCCCCCGGCAAGGGACGCGAGUUUGUGGCCGCAAUCACGUGGACAGCAGGCC |
| | | UAACCUACUACGCUGACUCCGUGAAGGGCAGGUUCACAAUUUCUAAAGACCGCGCCAA |
| | | GAACACCGUGGAUCUGCAGAUGAACUCCCUGCGCCCCGAGGACACAGCCCUGUACUAC |
| | | UGCGCCUCACGGCGCACGUUCUCAUACGAGCACCCCAAUUACUAUACCAGGUCAGCCU |
| | | ACCACUUUUGGGGACAGGGCACCCUGGUGACCGUGUCCUCCGCCGCCGCUAUCGAGGU |
| | | GAUGUACCCUCCCCCUUACCUGGACAACGAGAAAUCAAACGGCACCAUCAUCCACGUG |
| | | AAGGGAAAGCACCUGUGUCCUAGCCCACUGUUCCCAGGCCCCAGCAAGCCCUUUUGGG |
| | | UGCUGGUUGUGGUGGGCGGCGUGCUGGCCUGCUACAGCCUGCUGGUCACCGUGGCCQU |
| | | CAUCAUCUUCUGGAAGCGGGGACGCAAGAAGCUGCUGUAUAUCUUUAAGCAACCAUU |
| | | CAUGCGGCCCGUUCAAACGACUCAAGAAGAAGACGGGUGUAGCUGCCGGUUUCCCGAA |
| | | GAAGAAGAGGGCGGCUGUGAACUACGCGUGAAGUUCUCCAGGAGCGCCGAUGCCCCCG |
| | | CCUACCAGCAGGGCCAGAACCAGCUGUACAACGAGCUGAACCUGGGCCGGCGGGAGGA |
| | | AUACGACGUGCUGGACAAGCGGAGAGGCCGGGACCCUGAGAUGGGCGGAAAGCCUAG |
| | | ACGCAAGAACCCCCAGGAGGGCCUGUACAACGAGCUGCAGAAAGAUAAAAUGGCCGA |
| | | GGCCUAUAGCGAGAUCGGCAUGAAGGGCGAGAGAAGACGCGGCAAGGGGCACGAUGG |
| | | CCUGUACCAGGGGCUGAGCACCGCCACCAAAGACACCUACGAUGCCCUGCACAUGCAG |
| | | GCCCUGCCCCCGCGGUAAUAAUAA |
| 573 | Anti-CD19 CAR #1 mRNA | GGACAGAUCGCCUGGAGACGCCAUCCACGCUGUUUUGACCUCCAUAGAAGACACCGGG |
| | sequence (full RNA sequence) | ACCGAUCCAGCCUCCGCGGCCGGGAACGGUGCAUUGGAACGCGGAUUCCCCGUGCCAA |
| | | GAGUGACUCACCGUCCUUGACACGAUGGCUCUGCCCGUGACCGCACUGCUGCUGCCCC |
| | | UGGCCCUGCUGCUGCACGCCGCCAGGCCCGAGGUGCAGCUGGUGGAGUCCGGCGGCGG |
| | | CGUGGUGCAGCCAGGCGGCUCUCUGAGGCUGAGCUGCGCCGCCAGCGGCCUAUUUCUA |
| | | AGCGACUACACGCUAGCCUGGUUCAGGCAGGCCCCCGGCAAGGAGCGCGAGUUUGUGA |
| | | UCGUUAGCACGUGGAACCGCUAUACCUACUACGCUGAAGGAGUGAAGGGCAGGUUCA |
| | | CAAUUUCUCGGGACAACGCCAAGAAAACCGUGUAUCUGCAGAUGAACUCCCUGCGCCC |
| | | CGAGGACACAGCCCUGUACUACUGCGCCGCAAAAUUUCGCAUCUCAUACAACGUUAGG |
| | | GACGACAGCUUCUACGACCUGUGGGGACAGGGCACCCUGGUGACCGUGUCCUCCGCCG |
| | | CCGCUAUCGAGGUGAUGUACCCUCCCCCUUACCUGGACAACGAGAAAUCAAACGGCAC |
| | | CAUCAUCCACGUGAAGGGAAAGCACCUGUGUCCUAGCCCACUGUUCCCAGGCCCCAGC |
| | | AAGCCCUUUUGGGUGCUGGUUGUGGUGGGCGGCGUGCUGGCCUGCUACAGCCUGCUG |
| | | GUCACCGUGGCCUUCAUCAUCUUCUGGGUUCGGUCCAAGCGCUCACGCCUGCUACACU |
| | | CAGACUACAUGAACAUGACGCCCCGCAGACCUGGACCUACCAGGAAACACUAUCAACC |
| | | AUAUGCCCCCCCAAGGGACUUUGCAGCAUACAGGAGCCGCGUGAAGUUCUCCAGGAGC |
| | | GCCGAUGCCCCCGCCUACCAGCAGGGCCAGAACCAGCUGUACAACGAGCUGAACCUGG |
| | | GCCGGCGGGAGGAAUACGACGUGCUGGACAAGCGGAGAGGCCGGGACCCUGAGAUGG |
| | | GCGGAAAGCCUAGACGCAAGAACCCCCAGGAGGGCCUGUACAACGAGCUGCAGAAAGA |
| | | UAAAAUGGCCGAGGCCUAUAGCGAGAUCGGCAUGAAGGGCGAGAGAAGACGCGGCAA |
| | | GGGGCACGAUGGCCUGUACCAGGGGCUGAGCACCGCCACCAAAGACACCUACGAUGCC |
| | | CUGCACAUGCAGGCCCUGCCCCCGCGGUAAUAAUAACGGGUGGCAUCCCUGUGACCCC |
| | | UCCCCAGUGCCUCUCCUGGCCCUGGAAGUUGCCACUCCAGUGCCCACCAGCCUUGUCC |
| | | UAAUAAAAUUAAGUUGCAUC |
| 574 | Anti-CD19 CAR #2 mRNA | GGACAGAUCGCCUGGAGACGCCAUCCACGCUGUUUUGACCUCCAUAGAAGACACCGGG |
| | sequence (full RNA sequence) | ACCGAUCCAGCCUCCGCGGCCGGGAACGGUGCAUUGGAACGCGGAUUCCCCGUGCCAA |
| | | GAGUGACUCACCGUCCUUGACACGAUGGCUCUGCCCGUGACCGCACUGCUGCUGCCCC |
| | | UGGCCCUGCUGCUGCACGCCGCCAGGCCCGAGGUGCAGCUGGUGGAGUCCGGCGGCGG |
| | | CGUGGUGCAGCCAGGCGGCUCUCUGAGGCUGAGCUGCGCCGCCAGCGGCCUAUUUCUA |
| | | AGCGACUACACGCUAGCCUGGUUCAGGCAGGCCCCCGGCAAGGAGCGCGAGUUUGUGA |
| | | UCGUUAGCACGUGGAACCGCUAUACCUACUACGCUGAAGGAGUGAAGGGCAGGUUCA |
| | | CAAUUUCUCGGGACAACGCCAAGAAAACCGUGUAUCUGCAGAUGAACUCCCUGCGCCC |
| | | CGAGGACACAGCCCUGUACUACUGCGCCGCAAAAUUUCGCAUCUCAUACAACGUUAGG |
| | | GACGACAGCUUCUACGACCUGUGGGGACAGGGCACCCUGGUGACCGUGUCCUCCGCCG |
| | | CCGCUAUCGAGGUGAUGUACCCUCCCCCUUACCUGGACAACGAGAAAUCAAACGGCAC |
| | | CAUCAUCCACGUGAAGGGAAAGCACCUGUGUCCUAGCCCACUGUUCCCAGGCCCCAGC |
| | | AAGCCCUUUUGGGUGCUGGUUGUGGUGGGCGGCGUGCUGGCCUGCUACAGCCUGCUG |
| | | GUCACCGUGGCCUUCAUCAUCUUCUGGAAGCGGGGACGCAAGAAGCUGCUGUAUAUC |
| | | UUUAAGCAACCAUUCAUGCGGCCCGUUCAAACGACUCAAGAAGAAGACGGGUGUAGC |
| | | UGCCGGUUUCCCGAAGAAGAAGAGGGCGGCUGUGAACUACGCGUGAAGUUCUCCAGG |
| | | AGCGCCGAUGCCCCCGCCUACCAGCAGGGCCAGAACCAGCUGUACAACGAGCUGAACC |
| | | UGGGCCGGCGGGAGGAAUACGACGUGCUGGACAAGCGGAGAGGCCGGGACCCUGAGA |
| | | UGGGCGGAAAGCCUAGACGCAAGAACCCCCAGGAGGGCCUGUACAACGAGCUGCAGAA |
| | | AGAUAAAAUGGCCGAGGCCUAUAGCGAGAUCGGCAUGAAGGGCGAGAGAAGACGCGG |
| | | CAAGGGGCACGAUGGCCUGUACCAGGGGCUGAGCACCGCCACCAAAGACACCUACGAU |
| | | GCCCUGCACAUGCAGGCCCUGCCCCCGCGGUAAUAAUAACGGGUGGCAUCCCUGUGAC |
| | | CCCUCCCCAGUGCCUCUCCUGGCCCUGGAAGUUGCCACUCCAGUGCCCACCAGCCUUG |
| | | UCCUAAUAAAAUUAAGUUGCAUC |
| 575 | Anti-CD19 CAR #3 mRNA | GGACAGAUCGCCUGGAGACGCCAUCCACGCUGUUUUGACCUCCAUAGAAGACACCGGG |
| | sequence (full RNA sequence) | ACCGAUCCAGCCUCCGCGGCCGGGAACGGUGCAUUGGAACGCGGAUUCCCCGUGCCAA |
| | | GAGUGACUCACCGUCCUUGACACGAUGGCUCUGCCCGUGACCGCACUGCUGCUGCCCC |
| | | UGGCCCUGCUGCUGCACGCCGCCAGGCCCGAGGUGCAGCUGGUGGAGUCCGGCGGCGG |
| | | CGUGGUGCAGCCAGGCGGCUCUCUGAGGCUGAGCUGCGCCGCCAGCGGCGACACCUUC |
| | | GGAACGAAAGCCAUGGGAUGGUUCAGGCAGGCCCCCGGCAAGGGACGCGAGUUUGUG |
| | | GCCGCAAUCACGUGGACAGCAGGCCUAACCUACUACGCUGACUCCGUGAAGGGCAGGU |
| | | UCACAAUUUCUAAAGACCGCGCCAAGAACACCGUGGAUCUGCAGAUGAACUCCCUGCG |
| | | CCCCGAGGACACAGCCCUGUACUACUGCGCCUCACGGCGCACGUUCUCAUACGAGCAC |
| | | CCCAAUUACUAUACCAGGUCAGCCUACCACUUUUGGGGACAGGGCACCCUGGUGACCG |
| | | UGUCCUCCGCCGCCGCUAUCGAGGUGAUGUACCCUCCCCCUUACCUGGACAACGAGAA |
| | | AUCAAACGGCACCAUCAUCCACGUGAAGGGAAAGCACCUGUGUCCUAGCCCACUGUUC |
| | | CCAGGCCCCAGCAAGCCCUUUUGGGUGCUGGUUGUGGUGGGCGGCGUGCUGGCCUGCU |
| | | ACAGCCUGCUGGUCACCGUGGCCUUCAUCAUCUUCUGGAAGCGGGGACGCAAGAAGCU |
| | | GCUGUAUAUCUUUAAGCAACCAUUCAUGCGGCCCGUUCAAACGACUCAAGAAGAAGA |
| | | CGGGUGUAGCUGCCGGUUUCCCGAAGAAGAAGAGGGCGGCUGUGAACUACGCGUGAA |
| | | GUUCUCCAGGAGCGCCGAUGCCCCCGCCUACCAGCAGGGCCAGAACCAGCUGUACAAC |
| | | GAGCUGAACCUGGGCCGGCGGGAGGAAUACGACGUGCUGGACAAGCGGAGAGGCCGG |
| | | GACCCUGAGAUGGGCGGAAAGCCUAGACGCAAGAACCCCCAGGAGGGCCUGUACAACG |
| | | AGCUGCAGAAAGAUAAAAUGGCCGAGGCCUAUAGCGAGAUCGGCAUGAAGGGCGAGA |
| | | GAAGACGCGGCAAGGGGCACGAUGGCCUGUACCAGGGGCUGAGCACCGCCACCAAAGA |
| | | CACCUACGAUGCCCUGCACAUGCAGGCCCUGCCCCCGCGGUAAUAAUAACGGGUGGCA |
| | | UCCCUGUGACCCCUCCCCAGUGCCUCUCCUGGCCCUGGAAGUUGCCACUCCAGUGCCC |
| | | ACCAGCCUUGUCCUAAUAAAAUUAAGUUGCAUC |
| 576 | Anti-CD19 CAR #4 amino | METDTLLLWVLLLWVPGSTGEVKLQESGPGLVAPSQSLSVTCTVSGVSLPDYGVSWIRQPPR |
| | acid sequence | KGLEWLGVIWGSETTYYNSALKSRLTIIKDNSKSQVFLKMNSLQTDDTAIYYCAKHYYYGGS |
| | | YAMDYWGQGTSVTVSSGGGGSGGGGSGGGGSDIQMTQTTSSLSASLGDRVTISCRASQDISK |
| | | YLNWYQQKPDGTVKLLIYHTSRLHSGVPSRFSGSGSGTDYSLTISNLEQEDIATYFCQQGNTL |
| | | PYTFGGGTKLEIKAAAIEVMYPPPYLDNEKSNGTIIHVKGKHLCPSPLFPGPSKPFWVLVVVG |
| | | GVLACYSLLVTVAFIIFWVRSKRSRLLHSDYMNMTPRRPGPTRKHYQPYAPPRDFAAYRSRV |
| | | KFSRSADAPAYQQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 577 | Anti-CD19 CAR #5 amino | METDTLLLWVLLLWVPGSTGEVKLQESGPGLVAPSQSLSVTCTVSGVSLPDYGVSWIRQPPR |
| | acid sequence | KGLEWLGVIWGSETTYYNSALKSRLTIIKDNSKSQVFLKMNSLQTDDTAIYYCAKHYYYGGS |
| | | YAMDYWGQGTSVTVSSGGGGSGGGGSGGGGSDIQMTQTTSSLSASLGDRVTISCRASQDISK |
| | | YLNWYQQKPDGTVKLLIYHTSRLHSGVPSRFSGSGSGTDYSLTISNLEQEDIATYFCQQGNTL |
| | | PYTFGGGTKLEIKASTTTPAPRPPTPAPTIASQPLSLRPEACRPAAGGAVHTRGLDFACDIYIW |
| | | APLAGTCGVLLLSLVITLYCNHRNKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRRKNPQEG |
| | | LYNELQKDKMAEAYSEIGMKGERRRGKGHDGLYQGLSTATKDTYDALHMQALPPR |
| 578 | Anti-CD19 CAR #6 amino | METDTLLLWVLLLWVPGSTGQVQLVESGGGVVQPGRSLRLSCAASGFTFSSYGMHWVRQA |
| | acid sequence | PGKGLEWVAVIWYDGSNKYYVDSVKGRFTISRDNSKNTMYLQMNSLRAEDTAVYYCARRG |
| | | YNWNIAFDIWGQGTMVTVSSGGGGSGGGGSGGGGSGGGGSDIQMTQSPSTLSASVGDRVTI |
| | | TCRASQSISSWLAWYQQKPGKAPKLLIYKASSLENGVPSRFSGSGSGTEFTLTISSLQPDDFAT |
| | | YYCQQYNSYWTFGQGTKVEIKASSLRPEACRPAAGGAVHTRGLDFACDIYIWAPLAGTCGV |
| | | LLLSLVITLYCNHRNVRSKRSRLLHSDYMNMTPRRPGPTRKHYQPYAPPRDFAAYRSRVKFS |
| | | RSADAPAYQQGQNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| 579 | Anti-CD19 CAR #7 amino | METDTLLLWVLLLWVPGSTGQVQLVQSGAEVKKPGASVKVSCKASGYTFTSYGISWVRQAP |
| | acid sequence | GQGLEWMGWISAYNGNTNYAQKLQDRVTMTTHTSTSAAYMELRSLRSDDTAVYYCARDG |
| | | GLVAASPDYWGQGTLVTVSSGGGGSGGGGSGGGGSGGGGSDIVMTQSPDSLAVSLGERATI |
| | | NCKSSQSVLYNSNNKNYLAWYQQKPGQPPKLLIYRASTRDSGVPDRFSGSGSGTDFTLTISSL |
| | | QAEDVALYYCQQYYSTPYTFGQGTKLEIKASTTTPAPRPPTPAPTIASQPLSLRPEACRPAAGG |
| | | AVHTRGLDFACDIYIWAPLAGTCGVLLLSLVITLYCNHRNVRSKRSRLLHSDYMNMTPRRPG |
| | | PTRKHYQPYAPPRDFAAYRSRVKFSRSADAPAYQQGQNQLYNELNLGRREEYDVLDKRRGR |
| | | DPEMGGKPRRKNPQEGLYNELQKDKMAEAYSEIGMKGERRRGKGHDGLYQGLSTATKDTY |
| | | DALHMQALPPR |
| 580 | Anti-CD19 CAR #8 amino | METDTLLLWVLLLWVPGSTGEVQLVESGGGLVQAGESLRLSCIASGRTMSSYAMAWFRQAP |
| | acid sequence | GKEREFVAVSSLSGGTSDYADSVKGRFIISRDNARSTVTLQMNNLKPEDTAVYYCAARPVYI |
| | | RYSIRDYDIWGQGTLVTVSSASLSNSIMYFSHFVPVFLPAKPTTTPAPRPPTPAPTIASQPLSLR |
| | | PEACRPAAGGAVHTRGLDFACDIYIWAPLAGTCGVLLLSLVITLYCNHRNVRSKRSRLLHSD |
| | | YMNMTPRRPGPTRKHYQPYAPPRDFAAYRSRVKFSRSADAPAYQQGQNQLYNELNLGRREE |
| | | YDVLDKRRGRDPEMGGKPRRKNPQEGLYNELQKDKMAEAYSEIGMKGERRRGKGHDGLY |
| | | QGLSTATKDTYDALHMQALPPR |
| 581 | Anti-CD19 CAR #9 amino | METDTLLLWVLLLWVPGSTGEVQLVESGGGLVQAGESLRLSCIASGRTMSSYAMAWFRQAP |
| | acid sequence | GKEREFVAVSSLSGGTSDYADSVKGRFIISRDNARSTVTLQMNNLKPEDTAVYYCAARPVYI |
| | | RYSIRDYDIWGQGTLVTVSSASTTTPAPRPPTPAPTIASQPLSLRPEACRPAAGGAVHTRGLDF |
| | | ACDIYIWAPLAGTCGVLLLSLVITLYCNHRNVRSKRSRLLHSDYMNMTPRRPGPTRKHYQPY |
| | | APPRDFAAYRSRVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATKDTYDALHMQA |
| | | LPPR |
| 582 | Anti-CD19 CAR #10 amino | METDTLLLWVLLLWVPGSTGEVQLVESGGGLVQAGGSLRLSCAFSGRTFSSYAMGWFRQAP |
| | acid sequence | GKEREFVATISLSGGTTWYADSVKGRFTISRDNGASTAYLQMNSLKPEDTAVYYCAARPVY |
| | | HKTSARDYDFWGQGTQVTVSSASLSNSIMYFSHFVPVFLPAKPTTTPAPRPPTPAPTIASQPLS |
| | | LRPEACRPAAGGAVHTRGLDFACDIYIWAPLAGTCGVLLLSLVITLYCNHRNVRSKRSRLLH |
| | | SDYMNMTPRRPGPTRKHYQPYAPPRDFAAYRSRVKFSRSADAPAYQQGQNQLYNELNLGR |
| | | REEYDVLDKRRGRDPEMGGKPRRKNPQEGLYNELQKDKMAEAYSEIGMKGERRRGKGHD |
| | | GLYQGLSTATKDTYDALHMQALPPR |
| 583 | Anti-CD19 CAR #11 amino | METDTLLLWVLLLWVPGSTGEVQLVESGGGLVQAGGSLRLSCAFSGRTFSSYAMGWFRQAP |
| | acid sequence | GKEREFVATISLSGGTTWYADSVKGRFTISRDNGASTAYLQMNSLKPEDTAVYYCAARPVY |
| | | HKTSARDYDFWGQGTQVTVSSASTTTPAPRPPTPAPTIASQPLSLRPEACRPAAGGAVHTRGL |
| | | DFACDIYIWAPLAGTCGVLLLSLVITLYCNHRNVRSKRSRLLHSDYMNMTPRRPGPTRKHYQ |
| | | PYAPPRDFAAYRSRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLSTATKDTYDALHM |
| | | QALPPR |
| 584 | Anti-CD19 CAR #12 amino | METDTLLLWVLLLWVPGSTGEVQLVESGGGLVQAGGSLRLSCAASGFTFSSYAMAWFRQAP |
| | acid sequence | GKEREFVAVISLSGGSVDYRDSVKGRFTISRDNAQSTVSLQMNNLKPEDTAIYYCAARPVYM |
| | | KWSERDFDYWGQGTLVTVSSASLSNSIMYFSHFVPVFLPAKPTTTPAPRPPTPAPTIASQPLSL |
| | | RPEACRPAAGGAVHTRGLDFACDIYIWAPLAGTCGVLLLSLVITLYCNHRNVRSKRSRLLHS |
| | | DYMNMTPRRPGPTRKHYQPYAPPRDFAAYRSRVKFSRSADAPAYQQGQNQLYNELNLGRR |
| | | EEYDVLDKRRGRDPEMGGKPRRKNPQEGLYNELQKDKMAEAYSEIGMKGERRRGKGHDGL |
| | | YQGLSTATKDTYDALHMQALPPR |
| 385 | Anti-CD19 CAR #13 amino | METDTLLLWVLLLWVPGSTGEVQLVESGGGLVQAGGSLRLSCAASGFTFSSYAMAWFRQAP |
| | acid sequence | GKEREFVAVISLSGGSVDYRDSVKGRFTISRDNAQSTVSLQMNNLKPEDTAIYYCAARPVYM |
| | | KWSERDFDYWGQGTLVTVSSASTTTPAPRPPTPAPTIASQPLSLRPEACRPAAGGAVHTRGLD |
| | | FACDIYIWAPLAGTCGVLLLSLVITLYCNHRNVRSKRSRLLHSDYMNMTPRRPGPTRKHYQP |
| | | YAPPRDFAAYRSRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHMQ |
| | | ALPPR |
| 586 | Anti-CD19 CAR #14 amino | METDTLLLWVLLLWVPGSTGEVQLVESGGGLVQAGGSLKLACAASVHTSYTYGMGWFRQ |
| | acid sequence | APGKEREFVAVISSDGVRTNYADSVQGRFTISRDNAKNTIYLQMNNLKTEDSAIYYCAAGSG |
| | | LFSAGYEYQYWGQGTQVTVSSAAAIEVMYPPPYLDNEKSNGTIIHVKGKHLCPSPLFPGPSKP |
| | | FWVLVVVGGVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALHMQALPPR |
| 587 | Anti-CD19 CAR #15 amino | METDTLLLWVLLLWVPGSTGEVQLVESGGGLVQAGGSLKLACAASVHTSYTYGMGWFRQ |
| | acid sequence | APGKEREFVAVISSDGVRTNYADSVQGRFTISRDNAKNTIYLQMNNLKTEDSAIYYCAAGSG |
| | | LFSAGYEYQYWGQGTQVTVSSASTTTPAPRPPTPAPTIASQPLSLRPEACRPAAGGAVHTRGL |
| | | DFACDIYIWAPLAGTCGVLLLSLVITLYCNHRNVRSKRSRLLHSDYMNMTPRRPGPTRKHYQ |
| | | PYAPPRDFAAYRSRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLSTATKDTYDALHM |
| | | QALPPR |
| 588 | Anti-CD19 CAR #16 amino | METDTLLLWVLLLWVPGSTGEVQLVESGGGLVQAEGSLRLSCTASGLTFDNYAMGWFRQA |
| | acid sequence | PGKEREFVATISAAGHRTIFTNSVRGRFTASRDNAKKTTYLQMNSLKPEDTAVYYCAASPSQ |
| | | YGSMGKSAYDYWGQGTLVTVSSASLSNSIMYFSHFVPVFLPAKPTTTPAPRPPTPAPTIASQP |
| | | LSLRPEACRPAAGGAVHTRGLDFACDIYIWAPLAGTCGVLLLSLVITLYCNHRNVRSKRSRLL |
| | | HSDYMNMTPRRPGPTRKHYQPYAPPRDFAAYRSRVKFSRSADAPAYQQGQNQLYNELNLG |
| | | RREEYDVLDKRRGRDPEMGGKPRRKNPQEGLYNELQKDKMAEAYSEIGMKGERRRGKGHD |
| | | GLYQGLSTATKDTYDALHMQALPPR |
| 589 | Anti-CD19 CAR #17 amino | METDTLLLWVLLLWVPGSTGEVQLVESGGGLVQAEGSLRLSCTASGLTFDNYAMGWFRQA |
| | acid sequence | PGKEREFVATISAAGHRTIFTNSVRGRFTASRDNAKKTTYLQMNSLKPEDTAVYYCAASPSQ |
| | | YGSMGKSAYDYWGQGTLVTVSSASTTTPAPRPPTPAPTIASQPLSLRPEACRPAAGGAVHTR |
| | | GLDFACDIYIWAPLAGTCGVLLLSLVITLYCNHRNVRSKRSRLLHSDYMNMTPRRPGPTRKH |
| | | YQPYAPPRDFAAYRSRVKFSRSADAPAYQQGQNQLYNELNLGRREEYDVLDKRRGRDPEM |
| | | GGKPRRKNPQEGLYNELQKDKMAEAYSEIGMKGERRRGKGHDGLYQGLSTATKDTYDALH |
| | | MQALPPR |
| 590 | Anti-CD19 CAR #18 amino | METDTLLLWVLLLWVPGSTGEVQLVESGGGLVQAEGSLRLSCTASGLTFDNYAMGWFRQA |
| | acid sequence | PGKEREFVATISAAGHRTIFTNSVRGRFTASRDNAKKTTYLQMNSLKPEDTAVYYCAASPSQ |
| | | YGSMGKSAYDYWGQGTLVTVSSGGGGSGGGGSGGGGSEVQLVESGGGLVQAGDSLRLSCA |
| | | ASGRTLSSYTTGWFRQGLGKEREFVAAITKNGPYLYYKDSVRGRFTISRDSAKTTVFLQMDN |
| | | LKPEDTAVYYCAVSDAQYDIRNREKYYYWGQGTLVTVSSAAAIEVMYPPPYLDNEKSNGTII |
| | | HVKGKHLCPSPLFPGPSKPFWVLVVVGGVLACYSLLVTVAFIIFWVRSKRSRLLHSDYMNMT |
| | | PRRPGPTRKHYQPYAPPRDFAAYRSRVKFSRSADAPAYQQGQNQLYNELNLGRREEYDVLD |
| | | KRRGRDPEMGGKPRRKNPQEGLYNELQKDKMAEAYSEIGMKGERRRGKGHDGLYQGLSTA |
| | | TKDTYDALHMQALPPR |
| 591 | Anti-CD19 CAR #19 amino | MEETDTLLLWVLLLWVPGSTGEVQLVESGGGLVQAEGSLRLSCTASGLTFDNYAMGWFRQA |
| | acid sequence | PGKEREFVATISAAGHRTIFTNSVRGRFTASRDNAKKTTYLQMNSLKPEDTAVYYCAASPSQ |
| | | YGSMGKSAYDYWGQGTLVTVSSGGGGSGGGGSGGGGSEVQLVESGGGLVQPGGSLSLSCA |
| | | ASGDTFGTKAMGWFRQAPGKGREFVAAITWTAGLTYLADSAKGRFTISKDRVQNTVYLEM |
| | | NNLEPEDTAVYYCASRRTFSYEHPNYYTASAYHFWGQGTLVTVSSAAAIEVMYPPPYLDNE |
| | | KSNGTIIHVKGKHLCPSPLFPGPSKPFWVLVVVGGVLACYSLLVTVAFIIFWVRSKRSRLLHS |
| | | DYMNMTPRRPGPTRKHYQPYAPPRDFAAYRSRVKFSRSADAPAYQQGQNQLYNELNLGRR |
| | | EEYDVLDKRRGRDPEMGGKPRRKNPQEGLYNELQKDKMAEAYSEIGMKGERRRGKGHDGL |
| | | YQGLSTATKDTYDALHMQALPPR |
| 592 | Anti-CD19 CAR #20 amino | METDTLLLWVLLLWVPGSTGEVQLVESGGGLVQAEGSLRLSCTASGLTFDNYAMGWFRQA |
| | acid sequence | PGKEREFVATISAAGHRTIFTNSVRGRFTASRDNAKKTTYLQMNSLKPEDTAVYYCAASPSQ |
| | | YGSMGKSAYDYWGQGTLVTVSSGGGGSGGGGSGGGGSEVQLVESGGGLVQTGDSLRLTCA |
| | | ASGRTIGDRSMAWFRQAPGKEREFVIVNTWNGYTYTDESVEGRFTISRDSGANTVHLQMNK |
| | | LNPKDTAVYYCASSSYTYNIRQRSSYDWWGQGTLVTVSSAAAIEVMYPPPYLDNEKSNGTII |
| | | HVKGKHLCPSPLFPGPSKPFWVLVVVGGVLACYSLLVTVAFIIFWVRSKRSRLLHSDYMNMT |
| | | PRRPGPTRKHYQPYAPPRDFAAYRSRVKFSRSADAPAYQQGQNQLYNELNLGRREEYDVLD |
| | | KRRGRDPEMGGKPRRKNPQEGLYNELQKDKMAEAYSEIGMKGERRRGKGHDGLYQGLSTA |
| | | TKDTYDALHMQALPPR |
| 593 | Anti-CD19 CAR #21 amino | METDTLLLWVLLLWVPGSTGEVQLVESGGGLVQAGGSLRLSCAGSGFTFSSTVIGWYRQAP |
| | acid sequence | GKQREFVALITAGGHTNSVEDRFIISRDNAKSTAYLQMTSLKSEDTAVYYCNLPNQGYWGQ |
| | | GTLVTVSSAAAIEVMYPPPYLDNEKSNGTIIHVKGKHLCPSPLFPGPSKPFWVLVVVGGVLAC |
| | | YSLLVTVAFIIFWVRSKRSRLLHSDYMNMTPRRPGPTRKHYQPYAPPRDFAAYRSRVKFSRS |
| | | ADAPAYQQGQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 594 | Anti-CD19 CAR #22 amino | METDTLLLWVLLLWVPGSTGEVQLVESGGGLVQPGGSLRLFCAASGSRLSFTTMGWYRQAP |
| | acid sequence | GKQREVVAYITESGSTAYTASVKDRFTISRDNAKNTVSLQMNSLKPEDTAVYFCNAVIMMG |
| | | GSGTYWGPGTLVTVSSAAAIEVMYPPPYLDNEKSNGTIIHVKGKHLCPSPLFPGPSKPFWVLV |
| | | VVGGVLACYSLLVTVAFIIFWVRSKRSRLLHSDYMNMTPRRPGPTRKHYQPYAPPRDFAAYR |
| | | SRVKFSRSADAPAYQQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQALPPR |
| 595 | Anti-CD19 CAR #23 amino | MEETDTLLLWVLLLWVPGSTGEVQLVESGGGLVQAGDSLRLSCAASGRTLSSYTTGWFRQGL |
| | acid sequence | GKEREFVAAITKNGPYLYYKDSVRGRFTISRDSAKTTVFLQMDNLKPEDTAVYYCAVSDAQ |
| | | YDIRNREKYYYWGQGTLVTVSSAAAIEVMYPPPYLDNEKSNGTIIHVKGKHLCPSPLFPGPSK |
| | | PFWVLVVVGGVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALHMQALPPR |
| 596 | Anti-CD19 CAR #24 amino | METDTLLLWVLLLWVPGSTGEVQLVESGGGLVQAGDSLRLSCAASGRTLSSYTTGWFRQGL |
| | acid sequence | GKEREFVAAITKNGPYLYYKDSVRGRFTISRDSAKTTVFLQMDNLKPEDTAVYYCAVSDAQ |
| | | YDIRNREKYYYWGQGTLVTVSSASLSNSIMYFSHFVPVFLPAKPTTTPAPRPPTPAPTIASQPL |
| | | SLRPEACRPAAGGAVHTRGLDFACDIYIWAPLAGTCGVLLLSLVITLYCNHRNVRSKRSRLL |
| | | HSDYMNMTPRRPGPTRKHYQPYAPPRDFAAYRSRVKFSRSADAPAYQQGQNQLYNELNLG |
| | | RREEYDVLDKRRGRDPEMGGKPRRKNPQEGLYNELQKDKMAEAYSEIGMKGERRRGKGHD |
| | | GLYQGLSTATKDTYDALHMQALPPR |
| 597 | Anti-CD19 CAR #25 amino | METDTLLLWVLLLWVPGSTGEVQLVESGGGLVQAGDSLRLSCAASGRTLSSYTTGWFRQGL |
| | acid sequence | GKEREFVAAITKNGPYLYYKDSVRGRFTISRDSAKTTVFLQMDNLKPEDTAVYYCAVSDAQ |
| | | YDIRNREKYYYWGQGTLVTVSSGGGGSGGGGSGGGGSEVQLVESGGGLVQAEGSLRLSCTA |
| | | SGLTFDNYAMGWFRQAPGKEREFVATISAAGHRTIFTNSVRGRFTASRDNAKKTTYLQMNS |
| | | LKPEDTAVYYCAASPSQYGSMGKSAYDYWGQGTLVTVSSAAAIEVMYPPPYLDNEKSNGTII |
| | | HVKGKHLCPSPLFPGPSKPFWVLVVVGGVLACYSLLVTVAFIIFWVRSKRSRLLHSDYMNMT |
| | | PRRPGPTRKHYQPYAPPRDFAAYRSRVKFSRSADAPAYQQGQNQLYNELNLGRREEYDVLD |
| | | KRRGRDPEMGGKPRRKNPQEGLYNELQKDKMAEAYSEIGMKGERRRGKGHDGLYQGLSTA |
| | | TKDTYDALHMQALPPR |
| 598 | Anti-CD 19 CAR #26 amino | METDTLLLWVLLLWVPGSTGEVQLVESGGGLVQAGDSLRLSCAASGRTLSSYTTGWFRQGL |
| | acid sequence | GKEREFVAAITKNGPYLYYKDSVRGRFTISRDSAKTTVFLQMDNLKPEDTAVYYCAVSDAQ |
| | | YDIRNREKYYYWGQGTLVTVSSGGGGSGGGGSGGGGSEVQLVESGGGLVQTGDSLRLTCA |
| | | ASGRTIGDRSMAWFRQAPGKEREFVIVNTWNGYTYTDESVEGRFTISRDSGANTVHLQMNK |
| | | LNPKDTAVYYCASSSYTYNIRQRSSYDWWGQGTLVTVSSAAAIEVMYPPPYLDNEKSNGTII |
| | | HVKGKHLCPSPLFPGPSKPFWVLVVVGGVLACYSLLVTVAFIIFWVRSKRSRLLHSDYMNMT |
| | | PRRPGPTRKHYQPYAPPRDFAAYRSRVKFSRSADAPAYQQGQNQLYNELNLGRREEYDVLD |
| | | KRRGRDPEMGGKPRRKNPQEGLYNELQKDKMAEAYSEIGMKGERRRGKGHDGLYQGLSTA |
| | | TKDTYDALHMQALPPR |
| 599 | Anti-CD19 CAR #27 amino | MEETDTLLLWVLLLWVPGSTGEVQLVESGGGLVPPGGSLRLSCAASGRTGDINGMGWFRRAA |
| | acid sequence | GKEREFVAATRWNGGTKYYTSSVEGRFTISGDRVGNTVTLEMNNLVPEDTARYYCATTRRR |
| | | FDFYSVGGYENWGQGTQVTVSSASLSNSIMYFSHFVPVFLPAKPTTTPAPRPPTPAPTIASQPL |
| | | SLRPEACRPAAGGAVHTRGLDFACDIYIWAPLAGTCGVLLLSLVITLYCNHRNVRSKRSRLL |
| | | HSDYMNMTPRRPGPTRKHYQPYAPPRDFAAYRSRVKFSRSADAPAYQQGQNQLYNELNLG |
| | | RREEYDVLDKRRGRDPEMGGKPRRKNPQEGLYNELQKDKMAEAYSEIGMKGERRRGKGHD |
| | | GLYQGLSTATKDTYDALHMQALPPR |
| 600 | Anti-CD19 CAR #28 amino | METDTLLLWVLLLWVPGSTGEVQLVESGGGLVPPGGSLRLSCAASGRTGDINGMGWFRRAA |
| | acid sequence | GKEREFVAATRWNGGTKYYTSSVEGRFTISGDRVGNTVTLEMNNLVPEDTARYYCATTRRR |
| | | FDFYSVGGYENWGQGTQVTVSSASTTTPAPRPPTPAPTIASQPLSLRPEACRPAAGGAVHTRG |
| | | LDFACDIYIWAPLAGTCGVLLLSLVITLYCNHRNVRSKRSRLLHSDYMNMTPRRPGPTRKHY |
| | | QPYAPPRDFAAYRSRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGLSTATKDTYDALH |
| | | MQALPPR |
| 601 | Anti-CD19 CAR #29 amino | METDTLLLWVLLLWVPGSTGEVQLVESGGGLVQPGGSLSLSCAASGDTFGTKAMGWFRQA |
| | acid sequence | PGKGREFVAAITWTAGLTYLADSAKGRFTISKDRVQNTVYLEMNNLEPEDTAVYYCASRRT |
| | | FSYEHPNYYTASAYHFWGQGTLVTVSSAAAIEVMYPPPYLDNEKSNGTIIHVKGKHLCPSPLF |
| | | PGPSKPFWVLVVVGGVLACYSLLVTVAFIIFWVRSKRSRLLHSDYMNMTPRRPGPTRKHYQP |
| | | YAPPRDFAAYRSRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHMQ |
| | | ALPPR |
| 602 | Anti-CD19 CAR #30 amino | METDTLLLWVLLLWVPGSTGEVQLVESGGGLVQPGGSLSLSCAASGDTFGTKAMGWFRQA |
| | acid sequence | PGKGREFVAAITWTAGLTYLADSAKGRFTISKDRVQNTVYLEMNNLEPEDTAVYYCASRRT |
| | | FSYEHPNYYTASAYHFWGQGTLVTVSSGGGGSGGGGSGGGGSEVQLVESGGGLVQAEGSLR |
| | | LSCTASGLTFDNYAMGWFRQAPGKEREFVATISAAGHRTIFTNSVRGRFTASRDNAKKTTYL |
| | | QMNSLKPEDTAVYYCAASPSQYGSMGKSAYDYWGQGTLVTVSSAAAIEVMYPPPYLDNEK |
| | | SNGTIIHVKGKHLCPSPLFPGPSKPFWVLVVVGGVLACYSLLVTVAFIIFWVRSKRSRLLHSD |
| | | YMNMTPRRPGPTRKHYQPYAPPRDFAAYRSRVKFSRSADAPAYQQGQNQLYNELNLGRREE |
| | | YDVLDKRRGRDPEMGGKPRRKNPQEGLYNELQKDKMAEAYSEIGMKGERRRGKGHDGLY |
| | | QGLSTATKDTYDALHMQALPPR |
| 603 | Anti-CD19 CAR #31 amino | METDTLLLWVLLLWVPGSTGEVQLVESGGGLVQPGGSLRLSCVASGLFLSDYTLAWFRQAP |
| | acid sequence | GKEREFVIVSTWNGYTYYGESVSGRFTISRDNAEKTVFLQMNSLKPEDTAVYYCAAKFRISY |
| | | NVRDDSFYDLWGQGTLVTVSSAAAIEVMYPPPYLDNEKSNGTIIHVKGKHLCPSPLFPGPSKP |
| | | FWVLVVVGGVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALHMQALPPR |
| 604 | Anti-CD19 CAR #32 amino | METDTLLLWVLLLWVPGSTGEVQLVESGGGLVQPGGSLRLSCVASGLFLSDYTLAWFRQAP |
| | acid sequence | GKEREFVIVSTWNGYTYYGESVSGRFTISRDNAEKTVFLQMNSLKPEDTAVYYCAAKFRISY |
| | | NVRDDSFYDLWGQGTLVTVSSASTTTPAPRPPTPAPTIASQPLSLRPEACRPAAGGAVHTRGL |
| | | DFACDIYIWAPLAGTCGVLLLSLVITLYCNHRNKRGRKKLLYIFKQPFMRPVQTTQEEDGCSC |
| | | RFPEEEEGGCELRVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATKDTYDALHMQA |
| | | LPPR |
| 605 | Anti-CD19 CAR #33 amino | METDTLLLWVLLLWVPGSTGEVQLVESGGGLVQPGGSLRLSCVASGLFLSDYTLAWFRQAP |
| | acid sequence | GKEREFVIVSTWNGYTYYGESVSGRFTISRDNAEKTVFLQMNSLKPEDTAVYYCAAKFRISY |
| | | NVRDDSFYDLWGQGTLVTVSSASTTTPAPRPPTPAPTIASQPLSLRPEACRPAAGGAVHTRGL |
| | | DFACDIYIWAPLAGTCGVLLLSLVITLYCNHRNVRSKRSRLLHSDYMNMTPRRPGPTRKHYQ |
| | | PYAPPRDFAAYRSRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLSTATKDTYDALHM |
| | | QALPPR |
| 606 | Anti-CD19 CAR #34 amino | METDTLLLWVLLLWVPGSTGEVQLVESGGGLVQAGGSLTLTCAASGLIFAGYSVGWFRQA |
| | acid sequence | PGKEREFVAVITRGGDYRYFAESVQGRFTVSRDNGKNTVSLQMLDLKPDDTAVYYCAAGT |
| | | MTYAIRETRAYNYWGQGTLVTVSSASTTTPAPRPPTPAPTIASQPLSLRPEACRPAAGGAVHT |
| | | RGLDFACDIYIWAPLAGTCGVLLLSLVITLYCNHRNVRSKRSRLLHSDYMNMTPRRPGPTRK |
| | | HYQPYAPPRDFAAYRSRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQGLSTATKDTYDA |
| | | LHMQALPPR |
| 607 | Anti-CD19 CAR #35 amino | MEETDTLLLWVLLLWVPGSTGEVQLVESGGGLVQTGDSLRLTCAASGRTIGDRSMAWFRQAP |
| | acid sequence | GKEREFVIVNTWNGYTYTDESVEGRFTISRDSGANTVHLQMNKLNPKDTAVYYCASSSYTY |
| | | NIRQRSSYDWWGQGTLVTVSSAAAIEVMYPPPYLDNEKSNGTIIHVKGKHLCPSPLFPGPSKP |
| | | FWVLVVVGGVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALHMQALPPR |
| 608 | Anti-CD19 CAR #36 amino | METDTLLLWVLLLWVPGSTGEVQLVESGGGLVQTGDSLRLTCAASGRTIGDRSMAWFRQAP |
| | acid sequence | GKEREFVIVNTWNGYTYTDESVEGRFTISRDSGANTVHLQMNKLNPKDTAVYYCASSSYTY |
| | | NIRQRSSYDWWGQGTLVTVSSGGGGSGGGGSGGGGSEVQLVESGGGLVQAEGSLRLSCTAS |
| | | GLTFDNYAMGWFRQAPGKEREFVATISAAGHRTIFTNSVRGRFTASRDNAKKTTYLQMNSL |
| | | KPEDTAVYYCAASPSQYGSMGKSAYDYWGQGTLVTVSSAAAIEVMYPPPYLDNEKSNGTII |
| | | HVKGKHLCPSPLFPGPSKPFWVLVVVGGVLACYSLLVTVAFIIFWVRSKRSRLLHSDYMNMT |
| | | PRRPGPTRKHYQPYAPPRDFAAYRSRVKFSRSADAPAYQQGQNQLYNELNLGRREEYDVLD |
| | | KRRGRDPEMGGKPRRKNPQEGLYNELQKDKMAEAYSEIGMKGERRRGKGHDGLYQGLSTA |
| | | TKDTYDALHMQALPPR |
| 609 | Anti-CD19 CAR #37 amino | MEETDTLLLWVLLLWVPGSTGEVQLVESGGGLVQTGDSLRLTCAASGRTIGDRSMAWFRQAP |
| | acid sequence | GKEREFVIVNTWNGYTYTDESVEGRFTISRDSGANTVHLQMNKLNPKDTAVYYCASSSYTY |
| | | NIRQRSSYDWWGQGTLVTVSSGGGGSGGGGSGGGGSEVQLVESGGGLVQAGDSLRLSCAA |
| | | SGRTLSSYTTGWFRQGLGKEREFVAAITKNGPYLYYKDSVRGRFTISRDSAKTTVFLQMDNL |
| | | KPEDTAVYYCAVSDAQYDIRNREKYYYWGQGTLVTVSSAAAIEVMYPPPYLDNEKSNGTIIH |
| | | VKGKHLCPSPLFPGPSKPFWVLVVVGGVLACYSLLVTVAFIIFWVRSKRSRLLHSDYMNMTP |
| | | RRPGPTRKHYQPYAPPRDFAAYRSRVKFSRSADAPAYQQGQNQLYNELNLGRREEYDVLDK |
| | | RRGRDPEMGGKPRRKNPQEGLYNELQKDKMAEAYSEIGMKGERRRGKGHDGLYQGLSTAT |
| | | KDTYDALHMQALPPR |
| 610 | Anti-CD19 CAR #38 amino | MEETDTLLLWVLLLWVPGSTGEVQLVESGGGQVQAGGSLRLSCVHSGRTFSSYVAAWFRQY |
| | acid sequence | PGKEREFVAAASLSGGTTDYADSVKGRFTITRDNAASTHYLQMNSLQPEDTAVYYCAARPV |
| | | YLKYTYHDYDFWGQGTLVTVSSAAAIEVMYPPPYLDNEKSNGTIIHVKGKHLCPSPLFPGPS |
| | | KPFWVLVVVGGVLACYSLLVTVAFIIFWVRSKRSRLLHSDYMNMTPRRPGPTRKHYQPYAP |
| | | PRDFAAYRSRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALHMQALPP |
| | | R |
| 611 | Anti-CD19 CAR #39 amino | METDTLLLWVLLLWVPGSTGEVQLVESGGGLVQAGDSLRLSCSLSGRTFSSYAMAWFRQLP |
| | acid sequence | GKEREFVSVISLSGQNTYYADSVKGRFTISRDNSKSTVFLQMNSLKPEDTAVYYCAARPVYH |
| | | KFHYRDYDFWGQGTLVTVSSASTTTPAPRPPTPAPTIASQPLSLRPEACRPAAGGAVHTRGLD |
| | | FACDIYIWAPLAGTCGVLLLSLVITLYCNHRNVRSKRSRLLHSDYMNMTPRRPGPTRKHYQP |
| | | YAPPRDFAAYRSRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHMQ |
| | | ALPPR |
| 612 | Anti-CD19 CAR #40 amino | METDTLLLWVLLLWVPGSTGEVQLVESGGGLVQPGGSMRLSCQGSGRSFRLNTMVWYRRA |
| | acid sequence | SGNQRELVASLTSGGNTYYADSVKGRFTISRDDAKSTVDLQMNSLRPADTAVYFCYMGGNS |
| | | WGQETQVTVSSAAAIEVMYPPPYLDNEKSNGTIIHVKGKHLCPSPLFPGPSKPFWVLVVVGG |
| | | VLACYSLLVTVAFIIFWVRSKRSRLLHSDYMNMTPRRPGPTRKHYQPYAPPRDFAAYRSRVK |
| | | FSRSADAPAYQQGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQ |
| | | ALPPR |
| 613 | Anti-CD19 CAR #41 amino | MALPVTALLLPLALLLHAARPEVQLVESGGGVVQPGGSLRLSCAASGLFLSDYTLAWFRQAP |
| | acid sequence | GKEREFVIVSTWNRYTYYAEGVKGRFTISRDNAKKTVYLQMNSLRPEDTALYYCAAKFRISY |
| | | NVRDDSFYDLWGQGTLVTVSSAAAIEVMYPPPYLDNEKSNGTIIHVKGKHLCPSPLFPGPSKP |
| | | FWVLVVVGGVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALHMQALPPR |
| 614 | Anti-CD19 CAR #42 amino | MALPVTALLLPLALLLHAARPEVQLVESGGGVVQPGGSLRLSCAASGLFLSDYTLAWFRQAP |
| | acid sequence | GKEREFVIVSTWNRYTYYAEGVKGRFTISRDNAKKTVYLQMNSLRPEDTALYYCAAKFRISY |
| | | NVRDDSFYDLWGQGTLVTVSSAAAIEVMYPPPYLDNEKSNGTIIHVKGKHLCPSPLFPGPSKP |
| | | FWVLVVVGGVLACYSLLVTVAFIIFWKRGRKKLLYIFKQPFMRPVQTTQEEDGCSCRFPEEE |
| | | EGGCELRVKFSRSADAPAYQQGQNQLYNELNLGRREEYDVLDKRRGRDPEMGGKPRRKNP |
| | | QEGLYNELQKDKMAEAYSEIGMKGERRRGKGHDGLYQGLSTATKDTYDALHMQALPPR |
| 615 | Anti-CD19 CAR #43 amino | MALPVTALLLPLALLLHAARPEVQLVESGGGVVQPGGSLRLSCAASGDTFGTKAMGWFRQA |
| | acid sequence | PGKGREFVAAITWTAGLTYYADSVKGRFTISKDRAKNTVDLQMNSLRPEDTALYYCASRRT |
| | | FSYEHPNYYTRSAYHFWGQGTLVTVSSAAAIEVMYPPPYLDNEKSNGTIIHVKGKHLCPSPLF |
| | | PGPSKPFWVLVVVGGVLACYSLLVTVAFIIFWVRSKRSRLLHSDYMNMTPRRPGPTRKHYQP |
| | | YAPPRDFAAYRSRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHMQ |
| | | ALPPR |
| 616 | Anti-CD19 CAR #44 amino | MALPVTALLLPLALLLHAARPEVQLVESGGGVVQPGGSLRLSCAASGDTFGTKAMGWFRQA |
| | acid sequence | PGKGREFVAAITWTAGLTYYADSVKGRFTISKDRAKNTVDLQMNSLRPEDTALYYCASRRT |
| | | FSYEHPNYYTRSAYHFWGQGTLVTVSSAAAIEVMYPPPYLDNEKSNGTIIHVKGKHLCPSPLF |
| | | PGPSKPFWVLVVVGGVLACYSLLVTVAFIIFWKRGRKKLLYIFKQPFMRPVQTTQEEDGCSC |
| | | RFPEEEEGGCELRVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATKDTYDALHMQA |
| | | LPPR |
| 617 | Anti-CD19 CAR #45 amino | MALPVTALLLPLALLLHAARPEVQLVESGGGVVQPGGSLRLSCAASGFTFSSYAMAWFRQA |
| | acid sequence | PGKEREFVAVISLSGGSVDYRDSVKGRFTISRDNAKNTVDLQMNSLRPEDTALYYCAARPVY |
| | | IKWSERDFDYWGQGTLVTVSSAAAIEVMYPPPYLDNEKSNGTIIHVKGKHLCPSPLFPGPSKP |
| | | FWVLVVVGGVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALHMQALPPR |
| 618 | Anti-CD19 CAR #46 amino | MALPVTALLLPLALLLHAARPEVQLVESGGGVVQPGGSLRLSCAASGFTFSSYAMAWFRQA |
| | acid sequence | PGKEREFVAVISLSGGSVDYRDSVKGRFTISRDNAKNTVDLQMNSLRPEDTALYYCAARPVY |
| | | IKWSERDFDYWGQGTLVTVSSAAAIEVMYPPPYLDNEKSNGTIIHVKGKHLCPSPLFPGPSKP |
| | | FWVLVVVGGVLACYSLLVTVAFIIFWKRGRKKLLYIFKQPFMRPVQTTQEEDGCSCRFPEEE |
| | | EGGCELRVKFSRSADAPAYQQGQNQLYNELNLGRREEYDVLDKRRGRDPEMGGKPRRKNP |
| | | QEGLYNELQKDKMAEAYSEIGMKGERRRGKGHDGLYQGLSTATKDTYDALHMQALPPR |
| 619 | Anti-CD19 CAR #47 amino | MALPVTALLLPLALLLHAARPEVQLVESGGGVVQPGGSLRLSCAASGDTFGTKAMGWFRQA |
| | acid sequence | PGKGREFVAAITWTAGLTYYADSVKGRFTISKDRAKNTVDLQMNSLRPEDTALYYCASRRT |
| | | FSYEHPNYYTRSAYHFWGQGTLVTVSSAAAIEVMYPPPYLDNEKSNGTIIHVKGKHLCPSPLF |
| | | PGPSKPFWVLVVVGGVLACYSLLVTVAFIIFWVRSKRSRLLHSDYMNMTPRRPGPTRKHYQP |
| | | YAPPRDFAAYRSRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHMQ |
| | | ALPPR |
| 620 | Anti-CD19 CAR #48 amino | MALPVTALLLPLALLLHAARPEVQLVESGGGVVQPGGSLRLSCAASGFTFSSYAMAWFRQA |
| | acid sequence | PGKEREFVAVISLSGGSVDYRDSVKGRFTISRDNAKNTVDLQMNSLRPEDTALYYCAARPVY |
| | | IKWSERDFDYWGQGTLVTVSSAAAIEVMYPPPYLDNEKSNGTIIHVKGKHLCPSPLFPGPSKP |
| | | FWVLVVVGGVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTYDALHMQALPPR |
| 621 | Anti-CD19 CAR #49 amino | MALPVTALLLPLALLLHAARPEVQLVESGGGVVQPGGSLRLSCAASGFTFSSYAMAWFRQA |
| | acid sequence | PGKEREFVAVISLSGGSVDYRDSVKGRFTISRDNAKNTVDLQMNSLRPEDTALYYCAARPVY |
| | | IKWSERDFDYWGQGTLVTVSSAAAIEVMYPPPYLDNEKSNGTIIHVKGKHLCPSPLFPGPSKP |
| | | FWVLVVVGGVLACYSLLVTVAFIIFWKRGRKKLLYIFKQPFMRPVQTTQEEDGCSCRFPEEE |
| | | EGGCELRVKFSRSADAPAYQQGQNQLYNELNLGRREEYDVLDKRRGRDPEMGGKPRRKNP |
| | | QEGLYNELQKDKMAEAYSEIGMKGERRRGKGHDGLYQGLSTATKDTYDALHMQALPPR |
| 622 | Anti-CD19 CAR #50 amino | MILLLVTSLLLCELPHPAFLLIPDIQMTQTTSSLSASLGDRVTISCRASQDISKYLNWYQQKPDG |
| | acid sequence | TVKLLIYHTSRLHSGVPSRFSGSGSGTDYSLTISNLEQEDIATYFCQQGNTLPYTFGGGTKLEIT |
| | | GSTSGSGKPGSGEGSTKGEVKLQESGPGLVAPSQSLSVTCTVSGVSLPDYGVSWIRQPPRKGL |
| | | EWLGVIWGSETTYYNSALKSRLTIIKDNSKSQVFLKMNSLQTDDTAIYYCAKHYYYGGSYA |
| | | MDYWGQGTSVTVSSAAAIEVMYPPPYLDNEKSNGTIIHVKGKHLCPSPLFPGPSKPFWVLVV |
| | | VGGVLACYSLLVTVAFIIFWVRSKRSRLLHSDYMNMTPRRPGPTRKHYQPYAPPRDFAAYRS |
| | | RVKFSRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 623 | Anti-CD19 CAR #51 amino | MLLLVTSLLLCELPHPAFLLIPDIQMTQTTSSLSASLGDRVTISCRASQDISKYLNWYQQKPDG |
| | acid sequence | TVKLLIYHTSRLHSGVPSRFSGSGSGTDYSLTISNLEQEDIATYFCQQGNTLPYTFGGGTKLEIT |
| | | GSTSGSGKPGSGEGSTKGEVKLQESGPGLVAPSQSLSVTCTVSGVSLPDYGVSWIRQPPRKGL |
| | | EWLGVIWGSETTYYNSALKSRLTIIKDNSKSQVFLKMNSLQTDDTAIYYCAKHYYYGGSYA |
| | | MDYWGQGTSVTVSSAAAIEVMYPPPYLDNEKSNGTIIHVKGKHLCPSPLFPGPSKPFWVLVV |
| | | VGGVLACYSLLVTVAFIIFWVRSKRSRLLHSDYMNMTPRRPGPTRKHYQPYAPPRDFAAYRS |
| | | RVKFSRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 624 | Anti-CD19 CAR #4 amino | EVKLQESGPGLVAPSQSLSVTCTVSGVSLPDYGVSWIRQPPRKGLEWLGVIWGSETTYYNSA |
| | acid sequence (without | LKSRLTIIKDNSKSQVFLKMNSLQTDDTAIYYCAKHYYYGGSYAMDYWGQGTSVTVSSGGG |
| | signaling peptide) | GSGGGGSGGGGSDIQMTQTTSSLSASLGDRVTISCRASQDISKYLNWYQQKPDGTVKLLIYH |
| | | TSRLHSGVPSRFSGSGSGTDYSLTISNLEQEDIATYFCQQGNTLPYTFGGGTKLEIKAAAIEVM |
| | | YPPPYLDNEKSNGTIIHVKGKHLCPSPLFPGPSKPFWVLVVVGGVLACYSLLVTVAFIIFWVRS |
| | | KRSRLLHSDYMNMTPRRPGPTRKHYQPYAPPRDFAAYRSRVKFSRSADAPAYQQGQNQLYN |
| | | ELNLGRREEYDVLDKRRGRDPEMGGKPRRKNPQEGLYNELQKDKMAEAYSEIGMKGERRR |
| | | GKGHDGLYQGLSTATKDTYDALHMQALPPR |
| 625 | Anti-CD19 CAR #5 amino | EVKLQESGPGLVAPSQSLSVTCTVSGVSLPDYGVSWIRQPPRKGLEWLGVIWGSETTYYNSA |
| | acid sequence (without | LKSRLTIIKDNSKSQVFLKMNSLQTDDTAIYYCAKHYYYGGSYAMDYWGQGTSVTVSSGGG |
| | signaling peptide) | GSGGGGSGGGGSDIQMTQTTSSLSASLGDRVTISCRASQDISKYLNWYQQKPDGTVKLLIYH |
| | | TSRLHSGVPSRFSGSGSGTDYSLTISNLEQEDIATYFCQQGNTLPYTFGGGTKLEIKASTTTPAP |
| | | RPPTPAPTIASQPLSLRPEACRPAAGGAVHTRGLDFACDIYIWAPLAGTCGVLLLSLVITLYCN |
| | | HRNKRGRKKLLYIFKQPFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQKDKMAEAYSEIGMK |
| | | GERRRGKGHDGLYQGLSTATKDTYDALHMQALPPR |
| 626 | Anti-CD19 CAR #6 amino | QVQLVESGGGVVQPGRSLRLSCAASGFTFSSYGMHWVRQAPGKGLEWVAVIWYDGSNKYY |
| | acid sequence (without | VDSVKGRFTISRDNSKNTMYLQMNSLRAEDTAVYYCARRGYNWNIAFDIWGQGTMVTVSS |
| | signaling peptide) | GGGGSGGGGSGGGGSGGGGSDIQMTQSPSTLSASVGDRVTITCRASQSISSWLAWYQQKPG |
| | | KAPKLLIYKASSLENGVPSRFSGSGSGTEFTLTISSLQPDDFATYYCQQYNSYWTFGQGTKVEI |
| | | KASSLRPEACRPAAGGAVHTRGLDFACDIYIWAPLAGTCGVLLLSLVITLYCNHRNVRSKRS |
| | | RLLHSDYMNMTPRRPGPTRKHYQPYAPPRDFAAYRSRVKFSRSADAPAYQQGQNQLYNELN |
| | | LGRREEYDVLDKRRGRDPEMGGKPRRKNPQEGLYNELQKDKMAEAYSEIGMKGERRRGKG |
| | | HDGLYQGLSTATKDTYDALHMQALPPR |
| 627 | Anti-CD19 CAR #7 amino | QVQLVQSGAEVKKPGASVKVSCKASGYTFTSYGISWVRQAPGQGLEWMGWISAYNGNTNY |
| | acid sequence (without | AQKLQDRVTMTTHTSTSAAYMELRSLRSDDTAVYYCARDGGLVAASPDYWGQGTLVTVSS |
| | signaling peptide) | GGGGSGGGGSGGGGSGGGGSDIVMTQSPDSLAVSLGERATINCKSSQSVLYNSNNKNYLAW |
| | | YQQKPGQPPKLLIYRASTRDSGVPDRFSGSGSGTDFTLTISSLQAEDVALYYCQQYYSTPYTF |
| | | GQGTKLEIKASTTTPAPRPPTPAPTIASQPLSLRPEACRPAAGGAVHTRGLDFACDIYIWAPLA |
| | | GTCGVLLLSLVITLYCNHRNVRSKRSRLLHSDYMNMTPRRPGPTRKHYQPYAPPRDFAAYRS |
| | | RVKFSRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 628 | Anti-CD19 CAR #8 amino | EVQLVESGGGLVQAGESLRLSCIASGRTMSSYAMAWFRQAPGKEREFVAVSSLSGGTSDYA |
| | acid sequence (without | DSVKGRFIISRDNARSTVTLQMNNLKPEDTAVYYCAARPVYIRYSIRDYDIWGQGTLVTVSS |
| | signaling peptide) | ASLSNSIMYFSHFVPVFLPAKPTTTPAPRPPTPAPTIASQPLSLRPEACRPAAGGAVHTRGLDF |
| | | ACDIYIWAPLAGTCGVLLLSLVITLYCNHRNVRSKRSRLLHSDYMNMTPRRPGPTRKHYQPY |
| | | APPRDFAAYRSRVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATKDTYDALHMQA |
| | | LPPR |
| 629 | Anti-CD19 CAR #9 amino | EVQLVESGGGLVQAGESLRLSCIASGRTMSSYAMAWFRQAPGKEREFVAVSSLSGGTSDYA |
| | acid sequence (without | DSVKGRFIISRDNARSTVTLQMNNLKPEDTAVYYCAARPVYIRYSIRDYDIWGQGTLVTVSS |
| | signaling peptide) | ASTTTPAPRPPTPAPTIASQPLSLRPEACRPAAGGAVHTRGLDFACDIYIWAPLAGTCGVLLLS |
| | | LVITLYCNHRNVRSKRSRLLHSDYMNMTPRRPGPTRKHYQPYAPPRDFAAYRSRVKFSRSAD |
| | | APAYQQGQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 630 | Anti-CD19 CAR #10 amino | EVQLVESGGGLVQAGGSLRLSCAFSGRTFSSYAMGWFRQAPGKEREFVATISLSGGTTWYA |
| | acid sequence (without | DSVKGRFTISRDNGASTAYLQMNSLKPEDTAVYYCAARPVYHKTSARDYDFWGQGTQVTV |
| | signaling peptide) | SSASLSNSIMYFSHFVPVFLPAKPTTTPAPRPPTPAPTIASQPLSLRPEACRPAAGGAVHTRGLD |
| | | FACDIYIWAPLAGTCGVLLLSLVITLYCNHRNVRSKRSRLLHSDYMNMTPRRPGPTRKHYQP |
| | | YAPPRDFAAYRSRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHMQ |
| | | ALPPR |
| 631 | Anti-CD19 CAR #11 amino | EVQLVESGGGLVQAGGSLRLSCAFSGRTFSSYAMGWFRQAPGKEREFVATISLSGGTTWYA |
| | acid sequence (without | DSVKGRFTISRDNGASTAYLQMNSLKPEDTAVYYCAARPVYHKTSARDYDFWGQGTQVTV |
| | signaling peptide) | SSASTTTPAPRPPTPAPTIASQPLSLRPEACRPAAGGAVHTRGLDFACDIYIWAPLAGTCGVLL |
| | | LSLVITLYCNHRNVRSKRSRLLHSDYMNMTPRRPGPTRKHYQPYAPPRDFAAYRSRVKFSRS |
| | | ADAPAYQQGQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 632 | Anti-CD19 CAR #12 amino | EVQLVESGGGLVQAGGSLRLSCAASGFTFSSYAMAWFRQAPGKEREFVAVISLSGGSVDYR |
| | acid sequence (without | DSVKGRFTISRDNAQSTVSLQMNNLKPEDTAIYYCAARPVYMKWSERDFDYWGQGTLVTVS |
| | signaling peptide) | SASLSNSIMYFSHFVPVFLPAKPTTTPAPRPPTPAPTIASQPLSLRPEACRPAAGGAVHTRGLDF |
| | | ACDIYIWAPLAGTCGVLLLSLVITLYCNHRNVRSKRSRLLHSDYMNMTPRRPGPTRKHYQPY |
| | | APPRDFAAYRSRVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATKDTYDALHMQA |
| | | LPPR |
| 633 | Anti-CD19 CAR #13 amino | EVQLVESGGGLVQAGGSLRLSCAASGFTFSSYAMAWFRQAPGKEREFVAVISLSGGSVDYR |
| | acid sequence (without | DSVKGRFTISRDNAQSTVSLQMNNLKPEDTAIYYCAARPVYMKWSERDFDYWGQGTLVTVS |
| | signaling peptide) | SASTTTPAPRPPTPAPTIASQPLSLRPEACRPAAGGAVHTRGLDFACDIYIWAPLAGTCGVLLL |
| | | SLVITLYCNHRNVRSKRSRLLHSDYMNMTPRRPGPTRKHYQPYAPPRDFAAYRSRVKFSRSA |
| | | DAPAYQQGQNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 634 | Anti-CD19 CAR #14 amino | EVQLVESGGGLVQAGGSLKLACAASVHTSYTYGMGWFRQAPGKEREFVAVISSDGVRTNY |
| | acid sequence (without | ADSVQGRFTISRDNAKNTIYLQMNNLKTEDSAIYYCAAGSGLFSAGYEYQYWGQGTQVTVS |
| | signaling peptide) | SAAAIEVMYPPPYLDNEKSNGTIIHVKGKHLCPSPLFPGPSKPFWVLVVVGGVLACYSLLVTV |
| | | AFIIFWVRSKRSRLLHSDYMNMTPRRPGPTRKHYQPYAPPRDFAAYRSRVKFSRSADAPAYQ |
| | | QGQNQLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 635 | Anti-CD19 CAR #15 amino | EVQLVESGGGLVQAGGSLKLACAASVHTSYTYGMGWFRQAPGKEREFVAVISSDGVRTNY |
| | acid sequence (without | ADSVQGRFTISRDNAKNTIYLQMNNLKTEDSAIYYCAAGSGLFSAGYEYQYWGQGTQVTVS |
| | signaling peptide) | SASTTTPAPRPPTPAPTIASQPLSLRPEACRPAAGGAVHTRGLDFACDIYIWAPLAGTCGVLLL |
| | | SLVITLYCNHRNVRSKRSRLLHSDYMNMTPRRPGPTRKHYQPYAPPRDFAAYRSRVKFSRSA |
| | | DAPAYQQGQNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 636 | Anti-CD19 CAR #16 amino | EVQLVESGGGLVQAEGSLRLSCTASGLTFDNYAMGWFRQAPGKEREFVATISAAGHRTIFTN |
| | acid sequence (without | SVRGRFTASRDNAKKTTYLQMNSLKPEDTAVYYCAASPSQYGSMGKSAYDYWGQGTLVTV |
| | signaling peptide) | SSASLSNSIMYFSHFVPVFLPAKPTTTPAPRPPTPAPTIASQPLSLRPEACRPAAGGAVHTRGLD |
| | | FACDIYIWAPLAGTCGVLLLSLVITLYCNHRNVRSKRSRLLHSDYMNMTPRRPGPTRKHYQP |
| | | YAPPRDFAAYRSRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHMQ |
| | | ALPPR |
| 637 | Anti-CD19 CAR #17 amino | EVQLVESGGGLVQAEGSLRLSCTASGLTFDNYAMGWFRQAPGKEREFVATISAAGHRTIFTN |
| | acid sequence (without | SVRGRFTASRDNAKKTTYLQMNSLKPEDTAVYYCAASPSQYGSMGKSAYDYWGQGTLVTV |
| | signaling peptide) | SSASTTTPAPRPPTPAPTIASQPLSLRPEACRPAAGGAVHTRGLDFACDIYIWAPLAGTCGVLL |
| | | LSLVITLYCNHRNVRSKRSRLLHSDYMNMTPRRPGPTRKHYQPYAPPRDFAAYRSRVKFSRS |
| | | ADAPAYQQGQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 638 | Anti-CD19 CAR #18 amino | EVQLVESGGGLVQAEGSLRLSCTASGLTFDNYAMGWFRQAPGKEREFVATISAAGHRTIFTN |
| | acid sequence (without | SVRGRFTASRDNAKKTTYLQMNSLKPEDTAVYYCAASPSQYGSMGKSAYDYWGQGTLVTV |
| | signaling peptide) | SSGGGGSGGGGSGGGGSEVQLVESGGGLVQAGDSLRLSCÅASGRTLSSYTTGWFRQGLGKE |
| | | REFVAAITKNGPYLYYKDSVRGRFTISRDSAKTTVFLQMDNLKPEDTAVYYCAVSDAQYDIR |
| | | NREKYYYWGQGTLVTVSSAAAIEVMYPPPYLDNEKSNGTIIHVKGKHLCPSPLFPGPSKPFW |
| | | VLVVVGGVLACYSLLVTVAFIIFWVRSKRSRLLHSDYMNMTPRRPGPTRKHYQPYAPPRDFA |
| | | AYRSRVKFSRSADAPAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDALHMQALPPR |
| 639 | Anti-CD19 CAR #19 amino | EVQLVESGGGLVQAEGSLRLSCTASGLTFDNYAMGWFRQAPGKEREFVATISAAGHRTIFTN |
| | acid sequence (without | SVRGRFTASRDNAKKTTYLQMNSLKPEDTAVYYCAASPSQYGSMGKSAYDYWGQGTLVTV |
| | signaling peptide) | SSGGGGSGGGGSGGGGSEVQLVESGGGLVQPGGSLSLSCAASGDTFGTKAMGWFRQAPGK |
| | | GREFVAAITWTAGLTYLADSAKGRFTISKDRVQNTVYLEMNNLEPEDTAVYYCASRRTFSYE |
| | | HPNYYTASAYHFWGQGTLVTVSSAAAIEVMYPPPYLDNEKSNGTIIHVKGKHLCPSPLFPGPS |
| | | KPFWVLVVVGGVLACYSLLVTVAFIIFWVRSKRSRLLHSDYMNMTPRRPGPTRKHYQPYAP |
| | | PRDFAAYRSRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALHMQALPP |
| | | R |
| 640 | Anti-CD19 CAR #20 amino | EVQLVESGGGLVQAEGSLRLSCTASGLTFDNYAMGWFRQAPGKEREFVATISAAGHRTIFTN |
| | acid sequence (without | SVRGRFTASRDNAKKTTYLQMNSLKPEDTAVYYCAASPSQYGSMGKSAYDYWGQGTLVTV |
| | signaling peptide) | SSGGGGSGGGGSGGGGSEVQLVESGGGLVQTGDSLRLTCAASGRTIGDRSMAWFRQAPGKE |
| | | REFVIVNTWNGYTYTDESVEGRFTISRDSGANTVHLQMNKLNPKDTAVYYCASSSYTYNIRQ |
| | | RSSYDWWGQGTLVTVSSAAAIEVMYPPPYLDNEKSNGTIIHVKGKHLCPSPLFPGPSKPFWV |
| | | LVVVGGVLACYSLLVTVAFIIFWVRSKRSRLLHSDYMNMTPRRPGPTRKHYQPYAPPRDFAA |
| | | YRSRVKFSRSADAPAYQQGQNQLYNELNLGRREEYDVLDKRRGRDPEMGGKPRRKNPQEG |
| | | LYNELQKDKMAEAYSEIGMKGERRRGKGHDGLYQGLSTATKDTYDALHMQALPPR |
| 641 | Anti-CD19 CAR #21 amino | EVQLVESGGGLVQAGGSLRLSCAGSGFTFSSTVIGWYRQAPGKQREFVALITAGGHTNSVED |
| | acid sequence (without | RFIISRDNAKSTAYLQMTSLKSEDTAVYYCNLPNQGYWGQGTLVTVSSAAAIEVMYPPPYLD |
| | signaling peptide) | NEKSNGTIIHVKGKHLCPSPLFPGPSKPFWVLVVVGGVLACYSLLVTVAFIIFWVRSKRSRLL |
| | | HSDYMNMTPRRPGPTRKHYQPYAPPRDFAAYRSRVKFSRSADAPAYQQGQNQLYNELNLG |
| | | RREEYDVLDKRRGRDPEMGGKPRRKNPQEGLYNELQKDKMAEAYSEIGMKGERRRGKGHD |
| | | GLYQGLSTATKDTYDALHMQALPPR |
| 642 | Anti-CD19 CAR #22 amino | EVQLVESGGGLVQPGGSLRLFCAASGSRLSFTTMGWYRQAPGKQREVVAYITESGSTAYTAS |
| | acid sequence (without | VKDRFTISRDNAKNTVSLQMNSLKPEDTAVYFCNAVIMMGGSGTYWGPGTLVTVSSAAAIE |
| | signaling peptide) | VMYPPPYLDNEKSNGTIIHVKGKHLCPSPLFPGPSKPFWVLVVVGGVLACYSLLVTVAFIIFW |
| | | VRSKRSRLLHSDYMNMTPRRPGPTRKHYQPYAPPRDFAAYRSRVKFSRSADAPAYQQGQNQ |
| | | LYNELNLGRREEYDVLDKRRGRDPEMGGKPRRKNPQEGLYNELQKDKMAEAYSEIGMKGE |
| | | RRRGKGHDGLYQGLSTATKDTYDALHMQALPPR |
| 643 | Anti-CD19 CAR #23 amino | EVQLVESGGGLVQAGDSLRLSCAASGRTLSSYTTGWFRQGLGKEREFVAAITKNGPYLYYK |
| | acid sequence (without | DSVRGRFTISRDSAKTTVFLQMDNLKPEDTAVYYCAVSDAQYDIRNREKYYYWGQGTLVTV |
| | signaling peptide) | SSAAAIEVMYPPPYLDNEKSNGTIIHVKGKHLCPSPLFPGPSKPFWVLVVVGGVLACYSLLVT |
| | | VAFIIFWVRSKRSRLLHSDYMNMTPRRPGPTRKHYQPYAPPRDFAAYRSRVKFSRSADAPAY |
| | | QQGQNQLYNELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 644 | Anti-CD19 CAR #24 amino | EVQLVESGGGLVQAGDSLRLSCAASGRTLSSYTTGWFRQGLGKEREFVAAITKNGPYLYYK |
| | acid sequence (without | DSVRGRFTISRDSAKTTVFLQMDNLKPEDTAVYYCAVSDAQYDIRNREKYYYWGQGTLVTV |
| | signaling peptide) | SSASLSNSIMYFSHFVPVFLPAKPTTTPAPRPPTPAPTIASQPLSLRPEACRPAAGGAVHTRGLD |
| | | FACDIYIWAPLAGTCGVLLLSLVITLYCNHRNVRSKRSRLLHSDYMNMTPRRPGPTRKHYQP |
| | | YAPPRDFAAYRSRVKFSRSADAPAYQQGQNQLYNELNLGRREEYDVLDKRRGRDPEMGGK |
| | | PRRKNPQEGLYNELQKDKMAEAYSEIGMKGERRRGKGHDGLYQGLSTATKDTYDALHMQ |
| | | ALPPR |
| 645 | Anti-CD19 CAR #25 amino | EVQLVESGGGLVQAGDSLRLSCAASGRTLSSYTTGWFRQGLGKEREFVAAITKNGPYLYYK |
| | acid sequence (without | DSVRGRFTISRDSAKTTVFLQMDNLKPEDTAVYYCAVSDAQYDIRNREKYYYWGQGTLVTV |
| | signaling peptide) | SSGGGGSGGGGSGGGGSEVQLVESGGGLVQAEGSLRLSCTASGLTFDNYAMGWFRQAPGK |
| | | EREFVATISAAGHRTIFTNSVRGRFTASRDNAKKTTYLQMNSLKPEDTAVYYCAASPSQYGS |
| | | MGKSAYDYWGQGTLVTVSSAAAIEVMYPPPYLDNEKSNGTIIHVKGKHLCPSPLFPGPSKPF |
| | | WVLVVVGGVLACYSLLVTVAFIIFWVRSKRSRLLHSDYMNMTPRRPGPTRKHYQPYAPPRD |
| | | FAAYRSRVKFSRSADAPAYQQGQNQLYNELNLGRREEYDVLDKRRGRDPEMGGKPRRKNP |
| | | QEGLYNELQKDKMAEAYSEIGMKGERRRGKGHDGLYQGLSTATKDTYDALHMQALPPR |
| 646 | Anti-CD19 CAR #26 amino | EVQLVESGGGLVQAGDSLRLSCAASGRTLSSYTTGWFRQGLGKEREFVAAITKNGPYLYYK |
| | acid sequence (without | DSVRGRFTISRDSAKTTVFLQMDNLKPEDTAVYYCAVSDAQYDIRNREKYYYWGQGTLVTV |
| | signaling peptide) | SSGGGGSGGGGSGGGGSEVQLVESGGGLVQTGDSLRLTCAÅSGRTIGDRSMAWFRQAPGKE |
| | | REFVIVNTWNGYTYTDESVEGRFTISRDSGANTVHLQMNKLNPKDTAVYYCASSSYTYNIRQ |
| | | RSSYDWWGQGTLVTVSSAAAIEVMYPPPYLDNEKSNGTIIHVKGKHLCPSPLFPGPSKPFWV |
| | | LVVVGGVLACYSLLVTVAFIIFWVRSKRSRLLHSDYMNMTPRRPGPTRKHYQPYAPPRDFAA |
| | | YRSRVKFSRSADAPAYQQGQNQLYNELNLGRREEYDVLDKRRGRDPEMGGKPRRKNPQEG |
| | | LYNELQKDKMAEAYSEIGMKGERRRGKGHDGLYQGLSTATKDTYDALHMQALPPR |
| 647 | Anti-CD19 CAR #27 amino | EVQLVESGGGLVPPGGSLRLSCAASGRTGDINGMGWFRRAAGKEREFVAATRWNGGTKYY |
| | acid sequence (without | TSSVEGRFTISGDRVGNTVTLEMNNLVPEDTARYYCATTRRRFDFYSVGGYENWGQGTQVT |
| | signaling peptide) | VSSASLSNSIMYFSHFVPVFLPAKPTTTPAPRPPTPAPTIASQPLSLRPEACRPAAGGAVHTRGL |
| | | DFACDIYIWAPLAGTCGVLLLSLVITLYCNHRNVRSKRSRLLHSDYMNMTPRRPGPTRKHYQ |
| | | PYAPPRDFAAYRSRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLSTATKDTYDALHM |
| | | QALPPR |
| 648 | Anti-CD19 CAR #28 amino | EVQLVESGGGLVPPGGSLRLSCAASGRTGDINGMGWFRRAAGKEREFVAATRWNGGTKYY |
| | acid sequence (without | TSSVEGRFTISGDRVGNTVTLEMNNLVPEDTARYYCATTRRRFDFYSVGGYENWGQGTQVT |
| | signaling peptide) | VSSASTTTPAPRPPTPAPTIASQPLSLRPEACRPAAGGAVHTRGLDFACDIYIWAPLAGTCGVL |
| | | LLSLVITLYCNHRNVRSKRSRLLHSDYMNMTPRRPGPTRKHYQPYAPPRDFAAYRSRVKFSR |
| | | SADAPAYQQGQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 649 | Anti-CD19 CAR #29 amino | EVQLVESGGGLVQPGGSLSLSCAASGDTFGTKAMGWFRQAPGKGREFVAAITWTAGLTYLA |
| | acid sequence (without | DSAKGRFTISKDRVQNTVYLEMNNLEPEDTAVYYCASRRTFSYEHPNYYTASAYHFWGQGT |
| | signaling peptide) | LVTVSSAAAIEVMYPPPYLDNEKSNGTIIHVKGKHLCPSPLFPGPSKPFWVLVVVGGVLACYS |
| | | LLVTVAFIIFWVRSKRSRLLHSDYMNMTPRRPGPTRKHYQPYAPPRDFAAYRSRVKFSRSAD |
| | | APAYQQGQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 650 | Anti-CD19 CAR #30 amino | EVQLVESGGGLVQPGGSLSLSCAASGDTFGTKAMGWFRQAPGKGREFVAAITWTAGLTYLA |
| | acid sequence (without | DSAKGRFTISKDRVQNTVYLEMNNLEPEDTAVYYCASRRTFSYEHPNYYTASAYHFWGQGT |
| | signaling peptide) | LVTVSSGGGGSGGGGSGGGGSEVQLVESGGGLVQAEGSLRLSCTASGLTFDNYAMGWFRQ |
| | | APGKEREFVATISAAGHRTIFTNSVRGRFTASRDNAKKTTYLQMNSLKPEDTAVYYCAASPS |
| | | QYGSMGKSAYDYWGQGTLVTVSSAAAIEVMYPPPYLDNEKSNGTIIHVKGKHLCPSPLFPGP |
| | | SKPFWVLVVVGGVLACYSLLVTVAFIIFWVRSKRSRLLHSDYMNMTPRRPGPTRKHYQPYA |
| | | PPRDFAAYRSRVKFSRSADAPAYQQGQNQLYNELNLGRREEYDVLDKRRGRDPEMGGKPR |
| | | RKNPQEGLYNELQKDKMAEAYSEIGMKGERRRGKGHDGLYQGLSTATKDTYDALHMQALP |
| | | PR |
| 651 | Anti-CD19 CAR #31 amino | EVQLVESGGGLVQPGGSLRLSCVASGLFLSDYTLAWFRQAPGKEREFVIVSTWNGYTYYGES |
| | acid sequence (without | VSGRFTISRDNAEKTVFLQMNSLKPEDTAVYYCAAKFRISYNVRDDSFYDLWGQGTLVTVSS |
| | signaling peptide) | AAAIEVMYPPPYLDNEKSNGTIIHVKGKHLCPSPLFPGPSKPFWVLVVVGGVLACYSLLVTV |
| | | AFIIFWVRSKRSRLLHSDYMNMTPRRPGPTRKHYQPYAPPRDFAAYRSRVKFSRSADAPAYQ |
| | | QGQNQLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 652 | Anti-CD19 CAR #32 amino | EVQLVESGGGLVQPGGSLRLSCVASGLFLSDYTLAWFRQAPGKEREFVIVSTWNGYTYYGES |
| | acid sequence (without | VSGRFTISRDNAEKTVFLQMNSLKPEDTAVYYCAAKFRISYNVRDDSFYDLWGQGTLVTVSS |
| | signaling peptide) | ASTTTPAPRPPTPAPTIASQPLSLRPEACRPAAGGAVHTRGLDFACDIYIWAPLAGTCGVLLLS |
| | | LVITLYCNHRNKRGRKKLLYIFKQPFMRPVQTTQEEDGCSCRFPEEEEGGCELRVKFSRSADA |
| | | PAYQQGQNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 653 | Anti-CD19 CAR #33 amino | EVQLVESGGGLVQPGGSLRLSCVASGLFLSDYTLAWFRQAPGKEREFVIVSTWNGYTYYGES |
| | acid sequence (without | VSGRFTISRDNAEKTVFLQMNSLKPEDTAVYYCAAKFRISYNVRDDSFYDLWGQGTLVTVSS |
| | signaling peptide) | ASTTTPAPRPPTPAPTIASQPLSLRPEACRPAAGGAVHTRGLDFACDIYIWAPLAGTCGVLLLS |
| | | LVITLYCNHRNVRSKRSRLLHSDYMNMTPRRPGPTRKHYQPYAPPRDFAAYRSRVKFSRSAD |
| | | APAYQQGQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 654 | Anti-CD19 CAR #34 amino | EVQLVESGGGLVQAGGSLTLTCAASGLTFAGYSVGWFRQAPGKEREFVAVITRGGDYRYFA |
| | acid sequence (without | ESVQGRFTVSRDNGKNTVSLQMLDLKPDDTAVYYCAAGTMTYAIRETRAYNYWGQGTLVT |
| | signaling peptide) | VSSASTTTPAPRPPTPAPTIASQPLSLRPEACRPAAGGAVHTRGLDFACDIYIWAPLAGTCGVL |
| | | LLSLVITLYCNHRNVRSKRSRLLHSDYMNMTPRRPGPTRKHYQPYAPPRDFAAYRSRVKFSR |
| | | SADAPAYQQGQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 635 | Anti-CD19 CAR #35 amino | EVQLVESGGGLVQTGDSLRLTCAASGRTIGDRSMAWFRQAPGKEREFVIVNTWNGYTYTDE |
| | acid sequence (without | SVEGRFTISRDSGANTVHLQMNKLNPKDTAVYYCASSSYTYNIRQRSSYDWWGQGTLVTVS |
| | signaling peptide) | SAAAIEVMYPPPYLDNEKSNGTIIHVKGKHLCPSPLFPGPSKPFWVLVVVGGVLACYSLLVTV |
| | | AFIIFWVRSKRSRLLHSDYMNMTPRRPGPTRKHYQPYAPPRDFAAYRSRVKFSRSADAPAYQ |
| | | QGQNQLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 666 | Anti-CD 19 CAR #36 amino | EVQLVESGGGLVQTGDSLRLTCAASGRTIGDRSMAWFRQAPGKEREFVIVNTWNGYTYTDE |
| | acid sequence (without | SVEGRFTISRDSGANTVHLQMNKLNPKDTAVYYCASSSYTYNIRQRSSYDWWGQGTLVTVS |
| | signaling peptide) | SGGGGSGGGGSGGGGSEVQLVESGGGLVQAEGSLRLSCTASGLTFDNYAMGWFRQAPGKE |
| | | REFVATISAAGHRTIFTNSVRGRFTASRDNAKKTTYLQMNSLKPEDTAVYYCAASPSQYGSM |
| | | GKSAYDYWGQGTLVTVSSAAAIEVMYPPPYLDNEKSNGTIIHVKGKHLCPSPLFPGPSKPFW |
| | | VLVVVGGVLACYSLLVTVAFIIFWVRSKRSRLLHSDYMNMTPRRPGPTRKHYQPYAPPRDFA |
| | | AYRSRVKFSRSADAPAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDALHMQALPPR |
| 667 | Anti-CD19 CAR #37 amino | EVQLVESGGGLVQTGDSLRLTCAASGRTIGDRSMAWFRQAPGKEREFVIVNTWNGYTYTDE |
| | acid sequence (without | SVEGRFTISRDSGANTVHLQMNKLNPKDTAVYYCASSSYTYNIRQRSSYDWWGQGTLVTVS |
| | signaling peptide) | SGGGGSGGGGSGGGGSEVQLVESGGGLVQAGDSLRLSCAASGRTLSSYTTGWFRQGLGKER |
| | | EFVAAITKNGPYLYYKDSVRGRFTISRDSAKTTVFLQMDNLKPEDTAVYYCAVSDÅQYDIRN |
| | | REKYYYWGQGTLVTVSSAAAIEVMYPPPYLDNEKSNGTIIHVKGKHLCPSPLFPGPSKPFWV |
| | | LVVVGGVLACYSLLVTVAFIIFWVRSKRSRLLHSDYMNMTPRRPGPTRKHYQPYAPPRDFAA |
| | | YRSRVKFSRSADAPAYQQGQNQLYNELNLGRREEYDVLDKRRGRDPEMGGKPRRKNPQEG |
| | | LYNELQKDKMAEAYSEIGMKGERRRGKGHDGLYQGLSTATKDTYDALHMQALPPR |
| 668 | Anti-CD19 CAR #38 amino | EVQLVESGGGQVQAGGSLRLSCVHSGRTFSSYVAAWFRQYPGKEREFVAAASLSGGTTDYA |
| | acid sequence (without | DSVKGRFTITRDNAASTHYLQMNSLQPEDTAVYYCAARPVYLKYTYHDYDFWGQGTLVTV |
| | signaling peptide) | SSAAAIEVMYPPPYLDNEKSNGTIIHVKGKHLCPSPLFPGPSKPFWVLVVVGGVLACYSLLVT |
| | | VAFIIFWVRSKRSRLLHSDYMNMTPRRPGPTRKHYQPYAPPRDFAAYRSRVKFSRSADAPAY |
| | | QQGQNQLYNELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 669 | Anti-CD19 CAR #39 amino | EVQLVESGGGLVQAGDSLRLSCSLSGRTFSSYAMAWFRQLPGKEREFVSVISLSGQNTYYAD |
| | acid sequence (without | SVKGRFTISRDNSKSTVFLQMNSLKPEDTAVYYCAARPVYHKFHYRDYDFWGQGTLVTVSS |
| | signaling peptide) | ASTTTPAPRPPTPAPTIASQPLSLRPEACRPAAGGAVHTRGLDFACDIYIWAPLAGTCGVLLLS |
| | | LVITLYCNHRNVRSKRSRLLHSDYMNMTPRRPGPTRKHYQPYAPPRDFAAYRSRVKFSRSAD |
| | | APAYQQGQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 670 | Anti-CD19 CAR #40 amino | EVQLVESGGGLVQPGGSMRLSCQGSGRSFRLNTMVWYRRASGNQRELVASLTSGGNTYYA |
| | acid sequence (without | DSVKGRFTISRDDAKSTVDLQMNSLRPADTAVYFCYMGGNSWGQETQVTVSSAAAIEVMYP |
| | signaling peptide) | PPYLDNEKSNGTIIHVKGKHLCPSPLFPGPSKPFWVLVVVGGVLACYSLLVTVAFIIFWVRSK |
| | | RSRLLHSDYMNMTPRRPGPTRKHYQPYAPPRDFAAYRSRVKFSRSADAPAYQQGQNQLYNE |
| | | LNLGRREEYDVLDKRRGRDPEMGGKPRRKNPQEGLYNELQKDKMAEAYSEIGMKGERRRG |
| | | KGHDGLYQGLSTATKDTYDALHMQALPPR |
| 671 | Anti-CD19 CAR #41 amino | EVQLVESGGGVVQPGGSLRLSCAASGLFLSDYTLAWFRQAPGKEREFVIVSTWNRYTYYAE |
| | acid sequence (without | GVKGRFTISRDNAKKTVYLQMNSLRPEDTALYYCAAKFRISYNVRDDSFYDLWGQGTLVTV |
| | signaling peptide) | SSAAAIEVMYPPPYLDNEKSNGTIIHVKGKHLCPSPLFPGPSKPFWVLVVVGGVLACYSLLVT |
| | | VAFIIFWVRSKRSRLLHSDYMNMTPRRPGPTRKHYQPYAPPRDFAAYRSRVKFSRSADAPAY |
| | | QQGQNQLYNELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 672 | Anti-CD19 CAR #42 amino | EVQLVESGGGVVQPGGSLRLSCAASGLFLSDYTLAWFRQAPGKEREFVIVSTWNRYTYYAE |
| | acid sequence (without | GVKGRFTISRDNAKKTVYLQMNSLRPEDTALYYCAAKFRISYNVRDDSFYDLWGQGTLVTV |
| | signaling peptide) | SSAAAIEVMYPPPYLDNEKSNGTIIHVKGKHLCPSPLFPGPSKPFWVLVVVGGVLACYSLLVT |
| | | VAFIIFWKRGRKKLLYIFKQPFMRPVQTTQEEDGCSCRFPEEEEGGCELRVKFSRSADAPAYQ |
| | | QGQNQLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 673 | Anti-CD19 CAR #43 amino | EVQLVESGGGVVQPGGSLRLSCAASGDTFGTKAMGWFRQAPGKGREFVAAITWTAGLTYY |
| | acid sequence (without | ADSVKGRFTISKDRAKNTVDLQMNSLRPEDTALYYCASRRTFSYEHPNYYTRSAYHFWGQG |
| | signaling peptide) | TLVTVSSAAAIEVMYPPPYLDNEKSNGTIIHVKGKHLCPSPLFPGPSKPFWVLVVVGGVLACY |
| | | SLLVTVAFIIFWVRSKRSRLLHSDYMNMTPRRPGPTRKHYQPYAPPRDFAAYRSRVKFSRSA |
| | | DAPAYQQGQNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 674 | Anti-CD19 CAR #44 amino | EVQLVESGGGVVQPGGSLRLSCAASGDTFGTKAMGWFRQAPGKGREFVAAITWTAGLTYY |
| | acid sequence (without | ADSVKGRFTISKDRAKNTVDLQMNSLRPEDTALYYCASRRTFSYEHPNYYTRSAYHFWGQG |
| | signaling peptide) | TLVTVSSAAAIEVMYPPPYLDNEKSNGTIIHVKGKHLCPSPLFPGPSKPFWVLVVVGGVLACY |
| | | SLLVTVAFIIFWKRGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 675 | Anti-CD19 CAR #45 amino | EVQLVESGGGVVQPGGSLRLSCAASGFTFSSYAMAWFRQAPGKEREFVAVISLSGGSVDYRD |
| | acid sequence (without | SVKGRFTISRDNAKNTVDLQMNSLRPEDTALYYCAARPVYIKWSERDFDYWGQGTLVTVSS |
| | signaling peptide) | AAAIEVMYPPPYLDNEKSNGTIIHVKGKHLCPSPLFPGPSKPFWVLVVVGGVLACYSLLVTV |
| | | AFIIFWVRSKRSRLLHSDYMNMTPRRPGPTRKHYQPYAPPRDFAAYRSRVKFSRSADAPAYQ |
| | | QGQNQLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 676 | Anti-CD19 CAR #46 amino | EVQLVESGGGVVQPGGSLRLSCAASGFTFSSYAMAWFRQAPGKEREFVAVISLSGGSVDYRD |
| | acid sequence (without | SVKGRFTISRDNAKNTVDLQMNSLRPEDTALYYCAARPVYIKWSERDFDYWGQGTLVTVSS |
| | signaling peptide) | AAAIEVMYPPPYLDNEKSNGTIIHVKGKHLCPSPLFPGPSKPFWVLVVVGGVLACYSLLVTV |
| | | AFIIFWKRGRKKLLYIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 677 | Anti-CD19 CAR #47 amino | EVQLVESGGGVVQPGGSLRLSCAASGDTFGTKAMGWFRQAPGKGREFVAAITWTAGLTYY |
| | acid sequence (without | ADSVKGRFTISKDRAKNTVDLQMNSLRPEDTALYYCASRRTFSYEHPNYYTRSAYHFWGQG |
| | signaling peptide) | TLVTVSSAAAIEVMYPPPYLDNEKSNGTIIHVKGKHLCPSPLFPGPSKPFWVLVVVGGVLACY |
| | | SLLVTVAFIIFWVRSKRSRLLHSDYMNMTPRRPGPTRKHYQPYAPPRDFAAYRSRVKFSRSA |
| | | DAPAYQQGQNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| 678 | Anti-CD19 CAR #48 amino | EVQLVESGGGVVQPGGSLRLSCAASGFTFSSYAMAWFRQAPGKEREFVAVISLSGGSVDYRD |
| | acid sequence (without | SVKGRFTISRDNAKNTVDLQMNSLRPEDTALYYCAARPVYIKWSERDFDYWGQGTLVTVSS |
| | signaling peptide) | AAAIEVMYPPPYLDNEKSNGTIIHVKGKHLCPSPLFPGPSKPFWVLVVVGGVLACYSLLVTV |
| | | AFIIFWVRSKRSRLLHSDYMNMTPRRPGPTRKHYQPYAPPRDFAAYRSRVKFSRSADAPAYQ |
| | | QGQNQLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 679 | Anti-CD19 CAR #49 amino | EVQLVESGGGVVQPGGSLRLSCAASGFTFSSYAMAWFRQAPGKEREFVAVISLSGGSVDYRD |
| | acid sequence (without | SVKGRFTISRDNAKNTVDLQMNSLRPEDTALYYCAARPVYIKWSERDFDYWGQGTLVTVSS |
| | signaling peptide) | AAAIEVMYPPPYLDNEKSNGTIIHVKGKHLCPSPLFPGPSKPFWVLVVVGGVLACYSLLVTV |
| | | AFIIFWKRGRKKLLYIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 680 | Anti-CD19 CAR #50 amino | DIQMTQTTSSLSASLGDRVTISCRASQDISKYLNWYQQKPDGTVKLLIYHTSRLHSGVPSRFS |
| | acid sequence (without | GSGSGTDYSLTISNLEQEDIATYFCQQGNTLPYTFGGGTKLEITGSTSGSGKPGSGEGSTKGEV |
| | signaling peptide) | KLQESGPGLVAPSQSLSVTCTVSGVSLPDYGVSWIRQPPRKGLEWLGVIWGSETTYYNSALK |
| | | SRLTIIKDNSKSQVFLKMNSLQTDDTAIYYCAKHYYYGGSYAMDYWGQGTSVTVSSAAAIE |
| | | VMYPPPYLDNEKSNGTIIHVKGKHLCPSPLFPGPSKPFWVLVVVGGVLACYSLLVTVAFIIFW |
| | | VRSKRSRLLHSDYMNMTPRRPGPTRKHYQPYAPPRDFAAYRSRVKFSRSADAPAYQQGQNQ |
| | | LYNELNLGRREEYDVLDKRRGRDPEMGGKPRRKNPQEGLYNELQKDKMAEAYSEIGMKGE |
| | | RRRGKGHDGLYQGLSTATKDTYDALHMQALPPR |
| 681 | Anti-CD19 CAR #51 amino | DIQMTQTTSSLSASLGDRVTISCRASQDISKYLNWYQQKPDGTVKLLIYHTSRLHSGVPSRFS |
| | acid sequence (without | GSGSGTDYSLTISNLEQEDIATYFCQQGNTLPYTFGGGTKLEITGSTSGSGKPGSGEGSTKGEV |
| | signaling peptide) | KLQESGPGLVAPSQSLSVTCTVSGVSLPDYGVSWIRQPPRKGLEWLGVIWGSETTYYNSALK |
| | | SRLTIIKDNSKSQVFLKMNSLQTDDTAIYYCAKHYYYGGSYAMDYWGQGTSVTVSSAAAIE |
| | | VMYPPPYLDNEKSNGTIIHVKGKHLCPSPLFPGPSKPFWVLVVVGGVLACYSLLVTVAFIIFW |
| | | VRSKRSRLLHSDYMNMTPRRPGPTRKHYQPYAPPRDFAAYRSRVKFSRSADAPAYQQGQNQ |
| | | LYNELNLGRREEYDVLDKRRGRDPEMGGKPRRKNPQEGLYNELQKDKMAEAYSEIGMKGE |
| | | RRRGKGHDGLYQGLSTATKDTYDALHMQALPPR |
| 684 | VHH-C (VHH of CAR #4 and | EVKLQESGPGLVAPSQSLSVTCTVSGVSLPDYGVSWIRQPPRKGLEWLGVIWGSETTYYNSA |
| | CAR #5) | LKSRLTIIKDNSKSQVFLKMNSLQTDDTAIYYCAKHYYYGGSYAMDYWGQGTSVTVSS |
| 685 | VHH-D (VHH of CAR #6) | QVQLVESGGGVVQPGRSLRLSCAASGFTFSSYGMHWVRQAPGKGLEWVAVIWYDGSNKYY |
| | | VDSVKGRFTISRDNSKNTMYLQMNSLRAEDTAVYYCARRGYNWNIAFDIWGQGTMVTVSS |
| 686 | VHH-E (VHH of CAR #7) | QVQLVQSGAEVKKPGASVKVSCKASGYTFTSYGISWVRQAPGQGLEWMGWISAYNGNTNY |
| | | AQKLQDRVTMTTHTSTSAAYMELRSLRSDDTAVYYCARDGGLVAASPDYWGQGTLVTVSS |
| 687 | VHH-F (VHH of CAR #8 and | EVQLVESGGGLVQAGESLRLSCIASGRTMSSYAMAWFRQAPGKEREFVAVSSLSGGTSDYA |
| | CAR #9) | DSVKGRFIISRDNARSTVTLQMNNLKPEDTAVYYCAARPVYIRYSIRDYDIWGQGTLVTVSS |
| 688 | VHH-G (VHH of CAR #10 | EVQLVESGGGLVQAGGSLRLSCAFSGRTFSSYAMGWFRQAPGKEREFVATISLSGGTTWYA |
| | and CAR #11) | DSVKGRFTISRDNGASTAYLQMNSLKPEDTAVYYCAARPVYHKTSARDYDFWGQGTQVTV |
| | | SS |
| 699 | VHH-H (VHH of CAR #12 | EVQLVESGGGLVQAGGSLRLSCAASGFTFSSYAMAWFRQAPGKEREFVAVISLSGGSVDYR |
| | and CAR #13) | DSVKGRFTISRDNAQSTVSLQMNNLKPEDTAIYYCAARPVYMKWSERDFDYWGQGTLVTVS |
| | | S |
| 700 | VHH-I (VHH of CAR #14 | EVQLVESGGGLVQAGGSLKLACAASVHTSYTYGMGWFRQAPGKEREFVAVISSDGVRTNY |
| | and CAR #15) | ADSVQGRFTISRDNAKNTIYLQMNNLKTEDSAIYYCAAGSGLFSAGYEYQYWGQGTQVTVS |
| | | S |
| 701 | VHH-J (VHH of CAR #16 | EVQLVESGGGLVQAEGSLRLSCTASGLTFDNYAMGWFRQAPGKEREFVATISAAGHRTIFTN |
| | and CAR #17) | SVRGRFTASRDNAKKTTYLQMNSLKPEDTAVYYCAASPSQYGSMGKSAYDYWGQGTLVTV |
| | | SS |
| 702 | VHH-K (VHH of CAR #18) | EVQLVESGGGLVQAEGSLRLSCTASGLTFDNYAMGWFRQAPGKEREFVATISAAGHRTIFTN |
| | | SVRGRFTASRDNAKKTTYLQMNSLKPEDTAVYYCAASPSQYGSMGKSAYDYWGQGTLVTV |
| | | SSGGGGSGGGGSGGGGSEVQLVESGGGLVQAGDSLRLSCAASGRTLSSYTTGWFRQGLGKE |
| | | REFVAAITKNGPYLYYKDSVRGRFTISRDSAKTTVFLQMDNLKPEDTAVYYCAVSDAQYDIR |
| | | NREKYYYWGQGTLVTVSS |
| 703 | VHH-L (VHH of CAR #19) | EVQLVESGGGLVQAEGSLRLSCTASGLTFDNYAMGWFRQAPGKEREFVATISAAGHRTIFTN |
| | | SVRGRFTASRDNAKKTTYLQMNSLKPEDTAVYYCAASPSQYGSMGKSAYDYWGQGTLVTV |
| | | SSGGGGSGGGGSGGGGSEVQLVESGGGLVQPGGSLSLSCAASGDTFGTKAMGWFRQAPGK |
| | | GREFVAAITWTAGLTYLADSAKGRFTISKDRVQNTVYLEMNNLEPEDTAVYYCASRRTFSYE |
| | | HPNYYTASAYHFWGQGTLVTVSS |
| 704 | VHH-M (VHH of CAR #20) | EVQLVESGGGLVQAEGSLRLSCTASGLTFDNYAMGWFRQAPGKEREFVATISAAGHRTIFTN |
| | | SVRGRFTASRDNAKKTTYLQMNSLKPEDTAVYYCAASPSQYGSMGKSAYDYWGQGTLVTV |
| | | SSGGGGSGGGGSGGGGSEVQLVESGGGLVQTGDSLRLTCAASGRTIGDRSMAWFRQAPGKE |
| | | REFVIVNTWNGYTYTDESVEGRFTISRDSGANTVHLQMNKLNPKDTAVYYCASSSYTYNIRQ |
| | | RSSYDWWGQGTLVTVSS |
| 705 | VHH-N (VHH of CAR #21) | EVQLVESGGGLVQAGGSLRLSCAGSGFTFSSTVIGWYRQAPGKQREFVALITAGGHINSVED |
| | | RFIISRDNAKSTAYLQMTSLKSEDTAVYYCNLPNQGYWGQGTLVTVSS |
| 706 | VHH-O (VHH of CAR #22) | EVQLVESGGGLVQPGGSLRLFCAASGSRLSFTTMGWYRQAPGKQREVVAYITESGSTAYTAS |
| | | VKDRFTISRDNAKNTVSLQMNSLKPEDTAVYFCNAVIMMGGSGTYWGPGTLVTVSS |
| 707 | VHH-P (VHH of CAR #23 | EVQLVESGGGLVQAGDSLRLSCAASGRTLSSYTTGWFRQGLGKEREFVAAITKNGPYLYYK |
| | and CAR #24) | DSVRGRFTISRDSAKTTVFLQMDNLKPEDTAVYYCAVSDAQYDIRNREKYYYWGQGTLVTV |
| | | SS |
| 708 | VHH-Q (VHH of CAR #25) | EVQLVESGGGLVQAGDSLRLSCAASGRTLSSYTTGWFRQGLGKEREFVAAITKNGPYLYYK |
| | | DSVRGRFTISRDSAKTTVFLQMDNLKPEDTAVYYCAVSDAQYDIRNREKYYYWGQGTLVTV |
| | | SSGGGGSGGGGSGGGGSEVQLVESGGGLVQAEGSLRLSCTASGLTFDNYAMGWFRQAPGK |
| | | EREFVATISAAGHRTIFTNSVRGRFTASRDNAKKTTYLQMNSLKPEDTAVYYCAASPSQYGS |
| | | MGKSAYDYWGQGTLVTVSS |
| 709 | VHH-R (VHH of CAR #26) | EVQLVESGGGLVQAGDSLRLSCAASGRTLSSYTTGWFRQGLGKEREFVAAITKNGPYLYYK |
| | | DSVRGRFTISRDSAKTTVFLQMDNLKPEDTAVYYCAVSDAQYDIRNREKYYYWGQGTLVTV |
| | | SSGGGGSGGGGSGGGGSEVQLVESGGGLVQTGDSLRLTCAASGRTIGDRSMAWFRQAPGKE |
| | | REFVIVNTWNGYTYTDESVEGRFTISRDSGANTVHLQMNKLNPKDTAVYYCASSSYTYNIRQ |
| | | RSSYDWWGQGTLVTVSS |
| 710 | VHH-S (VHH of CAR #27 | EVQLVESGGGLVPPGGSLRLSCAASGRTGDINGMGWFRRAAGKEREFVAATRWNGGTKYY |
| | and CAR #28) | TSSVEGRFTISGDRVGNTVTLEMNNLVPEDTARYYCATTRRRFDFYSVGGYENWGQGTQVT |
| | | VSS |
| 711 | VHH-T (VHH of CAR #29) | EVQLVESGGGLVQPGGSLSLSCAASGDTFGTKAMGWFRQAPGKGREFVAAITWTAGLTYLA |
| | | DSAKGRFTISKDRVQNTVYLEMNNLEPEDTAVYYCASRRTFSYEHPNYYTASAYHFWGQGT |
| | | LVTVSS |
| 712 | VHH-U (VHH of CAR #30) | EVQLVESGGGLVQPGGSLSLSCAASGDTFGTKAMGWFRQAPGKGREFVAAITWTAGLTYLA |
| | | DSAKGRFTISKDRVQNTVYLEMNNLEPEDTAVYYCASRRTFSYEHPNYYTASAYHFWGQGT |
| | | LVTVSSGGGGSGGGGSGGGGSEVQLVESGGGLVQAEGSLRLSCTASGLTFDNYAMGWFRQ |
| | | APGKEREFVATISAAGHRTIFTNSVRGRFTASRDNAKKTTYLQMNSLKPEDTAVYYCAASPS |
| | | QYGSMGKSAYDYWGQGTLVTVSS |
| 713 | VHH-V (VHH of CAR #31, | EVQLVESGGGLVQPGGSLRLSCVASGLFLSDYTLAWFRQAPGKEREFVIVSTWNGYTYYGES |
| | CAR #32, and CAR #33) | VSGRFTISRDNAEKTVFLQMNSLKPEDTAVYYCAAKFRISYNVRDDSFYDLWGQGTLVTVSS |
| 714 | VHH-W (VHH of CAR #34) | EVQLVESGGGLVQAGGSLTLTCAASGLTFAGYSVGWFRQAPGKEREFVAVITRGGDYRYFA |
| | | ESVQGRFTVSRDNGKNTVSLQMLDLKPDDTAVYYCAAGTMTYAIRETRAYNYWGQGTLVT |
| | | VSS |
| 715 | VHH-X (VHH of CAR #35) | EVQLVESGGGLVQTGDSLRLTCAASGRTIGDRSMAWFRQAPGKEREFVIVNTWNGYTYTDE |
| | | SVEGRFTISRDSGANTVHLQMNKLNPKDTAVYYCASSSYTYNIRQRSSYDWWGQGTLVTVS |
| | | S |
| 716 | VHH-Y (VHH of CAR #36) | EVQLVESGGGLVQTGDSLRUTCAASGRTIGDRSMAWFRQAPGKEREFVIVNTWNGYTYTDE |
| | | SVEGRFTISRDSGANTVHLQMNKLNPKDTAVYYCASSSYTYNIRQRSSYDWWGQGTLVTVS |
| | | SGGGGSGGGGSGGGGSEVQLVESGGGLVQAEGSLRLSCTASGLTFDNYAMGWFRQAPGKE |
| | | REFVATISAAGHRTIFTNSVRGRFTASRDNAKKTTYLQMNSLKPEDTAVYYCAASPSQYGSM |
| | | GKSAYDYWGQGTLVTVSS |
| 717 | VHH-Z (VHH of CAR #37) | EVQLVESGGGLVQTGDSLRLTCAASGRTIGDRSMAWFRQAPGKEREFVIVNTWNGYTYTDE |
| | | SVEGRFTISRDSGANTVHLQMNKLNPKDTAVYYCASSSYTYNIRQRSSYDWWGQGTLVTVS |
| | | SGGGGSGGGGSGGGGSEVQLVESGGGLVQAGDSLRLSCAASGRTLSSYTTGWFRQGLGKER |
| | | EFVAAITKNGPYLYYKDSVRGRFTISRDSAKTTVFLQMDNLKPEDTAVYYCAVSDAQYDIRN |
| | | REKYYYWGQGTLVTVSS |
| 718 | VHH-AA (VHH of CAR #38) | EVQLVESGGGQVQAGGSLRLSCVHSGRTFSSYVAAWFRQYPGKEREFVAAASLSGGTTDYA |
| | | DSVKGRFTITRDNAASTHYLQMNSLQPEDTAVYYCAARPVYLKYTYHDYDFWGQGTLVTV |
| | | SS |
| 719 | VHH-AB (VHH of CAR #39) | EVQLVESGGGLVQAGDSLRLSCSLSGRTFSSYAMAWFRQLPGKEREFVSVISLSGQNTYYAD |
| | | SVKGRFTISRDNSKSTVFLQMNSLKPEDTAVYYCAARPVYHKFHYRDYDFWGQGTLVTVSS |
| 720 | VHH-AC (VHH of CAR #40) | EVQLVESGGGLVQPGGSMRLSCQGSGRSFRLNTMVWYRRASGNQRELVASLTSGGNTYYA |
| | | DSVKGRFTISRDDÅKSTVDLQMNSLRPADTAVYFCYMGGNSWGQETQVTVSS |
| 721 | VHH-AD (VHH of CAR | EVQLVESGGGVVQPGGSLRLSCAASGFTFSSYAMAWFRQAPGKEREFVAVISLSGGSVDYRD |
| | #45, CAR #46, CAR #48 and | SVKGRFTISRDNAKNTVDLQMNSLRPEDTALYYCAARPVYIKWSERDFDYWGQGTLVTVSS |
| | CAR #49) | |
| 722 | VHH-AE VHH of CAR #50 | DIQMTQTTSSLSASLGDRVTISCRASQDISKYLNWYQQKPDGTVKLLIYHTSRLHSGVPSRFS |
| | and CAR #51 | GSGSGTDYSLTISNLEQEDIATYFCQQGNTLPYTFGGGTKLEITGSTSGSGKPGSGEGSTKGEV |
| | | KLQESGPGLVAPSQSLSVTCTVSGVSLPDYGVSWIRQPPRKGLEWLGVIWGSETTYYNSALK |
| | | SRLTIIKDNSKSQVFLKMNSLQTDDTAIYYCAKHYYYGGSYAMDYWGQGTSVTVSS |
| 723 | VHH-AF | EVQLVESGGGVVQPGGSLRLSCAASGFTFSSYAMAWFRQAPGKEREFVAVISLSGGSVDYRD |
| | | SVKGRFTISRDNAQSTVSLQMNNLRPEDTALYYCAARPVYMKWSERDFDYWGQGTLVTVS |
| | | S |
| 724 | VHH-AG | EVQLVESGGGVVQPGGSLRLSCAASGFTFSSYAMAWFRQAPGKEREFVAVISLSGGSVDYA |
| | | DSVKGRFTISRDNAQSTVSLQMNSLRPEDTALYYCAARPVYMKWSERDFDYWGQGTLVTV |
| | | SS |
| 725 | VHH-AH | EVQLVESGGGVVQPGGSLRLSCAASGFTFSSYAMAWFRQAPGKEREFVAVISLSGGSVDYRD |
| | | SVKGRFTISRDNAKSTVSLQMNSLRPEDTALYYCAARPVYMKWSERDFDYWGQGTLVTVSS |
| 726 | VHH-AI | EVQLVESGGGVVQPGGSLRLSCAASGFTFSSYAMAWFRQAPGKEREFVAVISLSGGSVDYA |
| | | DSVKGRFTISRDNAKSTVSLQMNSLRPEDTALYYCAARPVYMKWSERDFDYWGQGTLVTV |
| | | SS |
| 727 | VHH-AJ | EVQLVESGGGVVQPGGSLRLSCAASGFTFSSYAMAWFRQAPGKEREFVAVISLSGGSVDYRD |
| | | SVKGRFTISRDNAQNTVSLQMNSLRPEDTALYYCAARPVYMKWSERDFDYWGQGTLVTVS |
| | | S |
| 728 | VHH-AK | EVQLVESGGGVVQPGGSLRLSCAASGFTFSSYAMAWFRQAPGKEREFVAVISLSGGSVDYA |
| | | DSVKGRFTISRDNAQNTVSLQMNSLRPEDTALYYCAARPVYMKWSERDFDYWGQGTLVTV |
| | | SS |
| 729 | VHH-AL | EVQLVESGGGVVQPGGSLRLSCAASGFTFSSYAMAWFRQAPGKEREFVAVISLSGGSVDYRD |
| | | SVKGRFTISRDNAKNTVSLQMNSLRPEDTALYYCAARPVYMKWSERDFDYWGQGTLVTVS |
| | | S |
| 730 | VHH-AM | EVQLVESGGGVVQPGGSLRLSCAASGFTFSSYAMAWFRQAPGKEREFVAVISLSGGSVDYA |
| | | DSVKGRFTISRDNAKNTVSLQMNSLRPEDTALYYCAARPVYMKWSERDFDYWGQGTLVTV |
| | | SS |
| 731 | VHH-AN | EVQLVESGGGVVQPGGSLRLSCAASGFTFSSYAMAWFRQAPGKEREFVAVISLSGGSVDYRD |
| | | SVKGRFTISRDNAQSTVYLQMNSLRPEDTALYYCAARPVYMKWSERDFDYWGQGTLVTVS |
| | | S |
| 732 | VHH-AO | EVQLVESGGGVVQPGGSLRLSCAASGFTFSSYAMAWFRQAPGKEREFVAVISLSGGSVDYRD |
| | | SVKGRFTISRDNAQSTVSLQMNSLRPEDTALYYCAARPVYIKWSERDFDYWGQGTLVTVSS |
| 733 | VHH-AP | EVQLVESGGGVVQPGGSLRLSCAASGFTFSSYAMAWFRQAPGKEREFVAVISLSGGSVDYRD |
| | | SVKGRFTISRDNAQSTVSLQMNSLRPEDTALYYCAARPVYLKWSERDFDYWGQGTLVTVSS |
| 734 | VHH-AQ | EVQLVESGGGVVQPGGSLRLSCAASGFTFSSYAMAWFRQAPGKEREFVAVISLSGGSVDYRD |
| | | SVKGRFTISRDNAQSTVSLQMNSLRPEDTALYYCAARPVYVKWSERDFDYWGQGTLVTVSS |
| 735 | VHH-AR | EVQLVESGGGVVQPGGSLRLSCAASGFTFSSYAMAWFRQAPGKEREFVAVISLSGGSVDYRD |
| | | SVKGRFTISRDNAQSTVSLQMNSLRPEDTALYYCAARPVYMKWSERDFDYWGQGTLVTVSS |
| 736 | VHH-AS | EVQLVESGGGVVQPGGSLRLSCAASGFTFSSYAMAWFRQAPGKEREFVAVISLSGGSVDYA |
| | | DSVKGRFTISRDNAKNTVSLQMNSLRPEDTALYYCAARPVYIKWSERDFDYWGQGTLVTVS |
| | | S |
| 737 | VHH-AT | EVQLVESGGGVVQPGGSLRLSCAASGFTFSSYAMAWFRQAPGKEREFVAVISLSGGSVDYRD |
| | | SVKGRFTISRDNAKNTVSLQMNSLRPEDTALYYCARRPVYIKWSERDFDYWGQGTLVTVSS |
| 738 | VHH-AU | EVQLVESGGGVVQPGGSLRLSCAASGFTFSSYAMAWFRQAPGKEREFVAVISLSGGSVDYRD |
| | | SVKGRFTISRDNAKNTVDLQMNSLRPEDTALYYCARRPVYIKWSERDFDYWGQGTLVTVSS |
| 739 | VHH-AV | EVQLVESGGGVVQPGGSLRLSCAASGFTFSSYAMAWFRQAPGKEREFVAVISLSGGSVDYRD |
| | | SVKGRFTISRDNAKNTVSLQMNSLRPEDTALYYCAARPVYIKWSERDFDYWGQGTLVTVSS |
| 740 | VHH-H H-CDR1 (Kabat | SYAMA |
| | numbering) | |
| 741 | VHH-H H-CDR2 (Kabat | VISLSGGSVDYRDSVKG |
| | numbering) | |
| 742 | VHH-AG H-CDR2 (Kabat | VISLSGGSVDYADSVKG |
| | numbering) | |
| 743 | VHH-H H-CDR3 (Kabat | RPVYMKWSERDFDY |
| | numbering) | |
| 744 | VHH-AD H-CDR3 (Kabat | RPVYIKWSERDFDY |
| | numbering) | |
| 745 | VHH-AP H-CDR3 (Kabat | RPVYLKWSERDFDY |
| | numbering) | |
| 746 | VHH-AQ H-CDR3 (Kabat | RPVYVKWSERDFDY |
| | numbering) | |
| 747 | VHH-H H-CDR1 (Abm | GFTFSSYAMA |
| | numbering) | |
| 748 | VHH-H H-CDR2 (Abm | VISLSGGSVD |
| | numbering) | |
| 749 | VHH-HH-CDR3 (Abm | RPVYMKWSERDFDY |
| | numbering) | |
| 750 | VHH-AD H-CDR3 (Abm | RPVYIKWSERDFDY |
| | numbering) | |
| 751 | VHH-AP H-CDR3 (Abm | RPVYLKWSERDFDY |
| | numbering) | |
| 752 | VHH-AQ H-CDR3 (Abm | RPVYVKWSERDFDY |
| | numbering) | |
| 753 | VHH-X-CDR1 (Kabat | DRSMA |
| | numbering) | |
| 754 | VHH-X H-CDR2 (Kabat | VNTWNGYTYTDESVEG |
| | numbering) | |
| 753 | VHH-X H-CDR3 (Kabat | SSYTYNIRQRSSYDW |
| | numbering) | |
| 756 | VHH-X H-CDR1 (Abm | GRTIGDRSMA |
| | numbering) | |
| 757 | VHH-X H-CDR2 (Abm | VNTWNGYTY |
| | numbering) | |
| 758 | VHH-X H-CDR3 (Abm | SSYTYNIRQRSSYDW |
| | numbering) | |
| 759 | VHH-W-CDR1 (Kabat | GYSVG |
| | numbering) | |
| 760 | VHH-W H-CDR2 (Kabat | VITRGGDYRYFAESVQG |
| | numbering) | |
| 761 | VHH-W H-CDR3 (Kabat | GTMTYAIRETRAYNY |
| | numbering) | |
| 762 | VHH-W H-CDR1 (Abm | GLTFAGYSVG |
| | numbering) | |
| 763 | VHH-W H-CDR2 (Abm | VITRGGDYRY |
| | numbering) | |
| 764 | VHH-W H-CDR3 (Abm | GTMTYAIRETRAYNY |
| | numbering) | |
| 765 | VHH-P-CDR1 (Kabat | SYTTG |
| | numbering) | |
| 766 | VHH-P H-CDR2 (Kabat | AITKNGPYLYYKDSVRG |
| | numbering) | |
| 767 | VHH-P H-CDR3 (Kabat | SDAQYDIRNREKYYY |
| | numbering) | |
| 768 | VHH-P H-CDR1 (Abm | GRTLSSYTTG |
| | numbering) | |
| 769 | VHH-P H-CDR2 (Abm | AITKNGPYLY |
| | numbering) | |
| 770 | VHH-P H-CDR3 (Abm | SDAQYDIRNREKYYY |
| | numbering) | |
| 771 | VHH-J-CDR1 (Kabat | NYAMG |
| | numbering) | |
| 772 | VHH-J H-CDR2 (Kabat | TISAAGHRTIFTNSVRG |
| | numbering) | |
| 773 | VHH-J H-CDR3 (Kabat | SPSQYGSMGKSAYDY |
| | numbering) | |
| 774 | VHH-J H-CDR1 (Abm | GLTFDNYAMG |
| | numbering) | |
| 775 | VHH-J H-CDR2 (Abm | TISAAGHRTI |
| | numbering) | |
| 776 | VHH-J H-CDR3 (Abm | SPSQYGSMGKSAYDY |
| | numbering) | |
| 777 | VHH-AA-CDR1 (Kabat | SYVAA |
| | numbering) | |
| 778 | VHH-AA H-CDR2 (Kabat | AASLSGGTTDYADSVKG |
| | numbering) | |
| 779 | VHH-AA H-CDR3 (Kabat | RPVYLKYTYHDYDF |
| | numbering) | |
| 780 | VHH-AA H-CDR1 (Abm | GRTFSSYVAA |
| | numbering) | |
| 781 | VHH-AA H-CDR2 (Abm | AASLSGGTTD |
| | numbering) | |
| 782 | VHH-AA H-CDR3 (Abm | RPVYLKYTYHDYDF |
| | numbering) | |
| 783 | VHH-O-CDR1 (Kabat | FTTMG |
| | numbering) | |
| 784 | VHH-O H-CDR2 (Kabat | YITESGSTAYTASVKD |
| | numbering) | |
| 785 | VHH-O H-CDR3 (Kabat | VIMMGGSGTY |
| | numbering) | |
| 786 | VHH-O H-CDR1 (Abm | GSRLSFTTMG |
| | numbering) | |
| 787 | VHH-O H-CDR2 (Abm | YITESGSTA |
| | numbering) | |
| 788 | VHH-O H-CDR3 (Abm | VIMMGGSGTY |
| | numbering) | |
| 789 | VHH-F CDR1 (Kabat | SYAMA |
| | numbering) | |
| 815 | VHH-F H-CDR2 (Kabat | VSSLSGGTSDYADSVKG |
| | numbering) | |
| 816 | VHH-F H-CDR3 (Kabat | RPVYIRYSIRDYDI |
| | numbering) | |
| 817 | VHH-F H-CDR1 (Abm | GRTMSSYAMA |
| | numbering) | |
| 818 | VHH-F H-CDR2 (Abm | VSSLSGGTSD |
| | numbering) | |
| 819 | VHH-F H-CDR3 (Abm | RPVYIRYSIRDYDI |
| | numbering) | |
| 820 | VHH-H FR1 (Kabat | EVQLVESGGGLVQAGGSLRLSCAASGFTFS |
| | numbering) | |
| 821 | VHH-H FR2 (Kabat | WFRQAPGKEREFVA |
| | numbering) | |
| 827 | VHH-H FR3 (Kabat | RFTISRDNAQSTVSLQMNNLKPEDTAIYYCAA |
| | numbering) | |
| 828 | VHH-H FR4 (Kabat | WGQGTLVTVSS |
| | numbering) | |
| 834 | VHH-AD FR1 (Kabat | EVQLVESGGGVVQPGGSLRLSCAASGFTFS |
| | numbering) | |
| 822 | VHH-AD FR2 (Kabat | WFRQAPGKEREFVA |
| | numbering) | |
| 790 | VHH-AD FR3 (Kabat | RFTISRDNAKNTVDLQMNSLRPEDTALYYCAA |
| | numbering) | |
| 829 | VHH-AD FR4 (Kabat | WGQGTLVTVSS |
| | numbering) | |
| 835 | VHH-AV FR1 (Kabat | EVQLVESGGGVVQPGGSLRLSCAASGFTFS |
| | numbering) | |
| 823 | VHH-AV FR2 (Kabat | WFRQAPGKEREFVA |
| | numbering) | |
| 791 | VHH-AV FR3 (Kabat | RFTISRDNAKNTVSLQMNSLRPEDTALYYCAA |
| | numbering) | |
| 830 | VHH-AV FR4 (Kabat | WGQGTLVTVSS |
| | numbering) | |
| 792 | VHH-H FR1 (Abm | EVQLVESGGGLVQAGGSLRLSCAAS |
| | numbering) | |
| 824 | VHH-H FR2 (Abm | WFRQAPGKEREFVA |
| | numbering) | |
| 793 | VHH-H FR3 (Abm | YRDSVKGRFTISRDNAQSTVSLQMNNLKPEDTAIYYCAA |
| | numbering) | |
| 831 | VHH-H FR4 (Abm | WGQGTLVTVSS |
| | numbering) | |
| 836 | VHH-AD FR1 (Abm | EVQLVESGGGVVQPGGSLRLSCAAS |
| | numbering) | |
| 825 | VHH-AD FR2 (Abm | WFRQAPGKEREFVA |
| | numbering) | |
| 794 | VHH-AD FR3 (Abm | YRDSVKGRFTISRDNAKNTVDLQMNSLRPEDTALYYCAA |
| | numbering) | |
| 832 | VHH-AD FR4 (Abm | WGQGTLVTVSS |
| | numbering) | |
| 837 | VHH-AV FR1 (Kabat | EVQLVESGGGVVQPGGSLRLSCAAS |
| | numbering) | |
| 826 | VHH-AV FR2 (Abm | WFRQAPGKEREFVA |
| | numbering) | |
| 795 | VHH-AV FR3 (Abm | YRDSVKGRFTISRDNAKNTVSLQMNSLRPEDTALYYCAA |
| | numbering) | |
| 833 | VHH-AV FR4 (Abm | WGQGTLVTVSS |
| | numbering) | |
| 796 | VHH FR2 (Kabat numbering) | RFTISRDNAQSTVSLQMNNLRPEDTALYYCAA |
| 797 | VHH FR3 (Kabat numbering) | RFTISRDNAQSTVSLQMNSLRPEDTALYYCAA |
| 798 | VHH FR3 (Kabat numbering) | RFTISRDNAKSTVSLQMNSLRPEDTALYYCAA |
| 799 | VHH FR3 (Kabat numbering) | RFTISRDNAKSTVSLQMNSLRPEDTALYYCAA |
| 800 | VHH FR3 (Kabat numbering) | RFTISRDNAKSTVSLQMNSLRPEDTALYYCAA |
| 801 | VHH FR3 (Kabat numbering) | RFTISRDNAQSTVYLQMNSLRPEDTALYYCAA |
| 802 | VHH FR3 (Kabat numbering) | RFTISRDNAKNTVSLQMNSLRPEDTALYYCAR |
| 803 | VHH FR3 (Kabat numbering) | RFTISRDNAKNTVDLQMNSLRPEDTALYYCAR |
| 804 | VHH FR3 (Abm numbering) | YRDSVKGRFTISRDNAQSTVSLQMNNLRPEDTALYYCAA |
| 805 | VHH FR3 (Abm numbering) | YADSVKGRFTISRDNAQSTVSLQMNSLRPEDTALYYCAA |
| 806 | VHH FR3 (Abm numbering) | YRDSVKGRFTISRDNAKSTVSLQMNSLRPEDTALYYCAA |
| 807 | VHH FR3 (Abm numbering) | YADSVKGRFTISRDNAKSTVSLQMNSLRPEDTALYYCAA |
| 808 | VHH FR3 (Abm numbering) | YRDSVKGRFTISRDNAQNTVSLQMNSLRPEDTALYYCAA |
| 809 | VHH FR3 (Abm numbering) | YADSVKGRFTISRDNAQNTVSLQMNSLRPEDTALYYCAA |
| 810 | VHH FR3 (Abm numbering) | YADSVKGRFTISRDNAKNTVSLQMNSLRPEDTALYYCAA |
| 811 | VHH FR3 (Abm numbering) | YRDSVKGRFTISRDNAQSTVYLQMNSLRPEDTALYYCAA |
| 812 | VHH FR3 (Abm numbering) | YRDSVKGRFTISRDNAQSTVSLQMNSLRPEDTALYYCAA |
| 813 | VHH FR3 (Abm numbering) | YRDSVKGRFTISRDNAKNTVSLQMNSLRPEDTALYYCAR |
| 814 | VHH FR3 (Abm numbering) | YRDSVKGRFTISRDNAKNTVDLQMNSLRPEDTALYYCAR |
| 838 | | RPVYX₁KWSERDFDY; X₁ is M, I, L, or V |
| 839 | | VISLSGGSVDYX₂DSVKG; X₂ is R or A |
| 840 | | METDTLLLWVLLLWVPGSTG |
| 841 | VHH-G-CDR1 (Kabat | SYAMG |
| | numbering) | |
| 842 | VHH-G-CDR2 (Kabat | TISLSGGTTWYADSVKG |
| | numbering) | |
| 843 | VHH-G-CDR3 (Kabat | RPVYHKTSARDYDF |
| | numbering) | |
| 844 | VHH-I-CDR1 (Kabat | TYGMG |
| | numbering) | |
| 845 | VHH-I-CDR2 (Kabat | VISSDGVRTNYADSVQG |
| | numbering) | |
| 846 | VHH-I-CDR3 (Kabat | GSGLFSAGYEYQY |
| | numbering) | |
| 847 | VHH-S-CDR1 (Kabat | INGMG |
| | numbering) | |
| 848 | VHH-S-CDR2 (Kabat | ATRWNGGTKYYTSSVEG |
| | numbering) | |
| 849 | VHH-S-CDR3 (Kabat | TRRRFDFYSVGGYEN |
| | numbering) | |
| 850 | VHH-T-CDR1 (Kabat | TKAMG |
| | numbering) | |
| 851 | VHH-T-CDR2 (Kabat | AITWTAGLTYLADSAKG |
| | numbering) | |
| 852 | VHH-T-CDR3 (Kabat | RRTFSYEHPNYYTASAYHF |
| | numbering) | |
| 853 | VHH-V-CDR1 (Kabat | DYTLA |
| | numbering) | |
| 854 | VHH-V-CDR2 (Kabat | VSTWNGYTYYGESVSG |
| | numbering) | |
| 429 | VHH-V-CDR3 (Kabat | KFRISYNVRDDSFYDL |
| | numbering) | |
| 740 | VHH-AB-CDR1 (Kabat | SYAMA |
| | numbering) | |
| 855 | VHH-AB-CDR2 (Kabat | VISLSGQNTYYADSVKG |
| | numbering) | |
| 856 | VHH-AB-CDR1 (Kabat | RPVYHKFHYRDYDF |
| | numbering) | |
| 857 | VHH-T-CDR1 (Abm | GDTFGTKAMG |
| | numbering) | |
| 858 | VHH-T-CDR2 (Abm | AITWTAGLTY |
| | numbering) | |
| 859 | VHH-T-CDR3 (Abm | RRTFSYEHPNYYTASAYHF |
| | numbering) | |

1: A polypeptide comprising an anti-CD19 chimeric antigen receptor (anti-CD19 CAR), wherein the anti-CD19 CAR comprises:
(a) an anti-CD19 antigen-binding domain, wherein the anti-CD19 antigen-binding domain is an anti-CD19 single domain antibody (anti-CD19 sdAb) domain comprising an anti-CD19 VHH comprising:
(i) an H-CDR1 comprising the amino acid sequence of SEQ ID NO:427, an H-CDR2 comprising the amino acid sequence of SEQ ID NO:428, and an H-CDR3 comprising the amino acid sequence of SEQ ID NO:429, or
(ii) an H-CDR1 comprising the amino acid sequence of SEQ ID NO:475, an H-CDR2 comprising the amino acid sequence of SEQ ID NO:476, and an H-CDR3 comprising the amino acid sequence of SEQ ID NO:477;
(b) a hinge domain,
(c) a transmembrane domain,
(d) an intracellular signaling domain, and
(e) optionally a co-stimulatory domain.

2: The polypeptide of claim 1, wherein the anti-CD19 sdAb comprises a VHH comprising the amino acid sequence of SEQ ID NO:433 or 481, or an amino acid sequence comprising at least about 80% sequence identity to the amino acid sequence of SEQ ID NO:433 or 481.

3: The polypeptide of claim 1, wherein the hinge domain is derived from a protein selected from the group consisting of CD28, CD5, CD9, CD2, CD27, CD28, IgG, IgK, and CD8α.

4: The polypeptide of claim 3, wherein in the hinge domain is derived from CD28 or CD8α.

5: The polypeptide of claim 1, wherein the transmembrane domain is derived from a protein selected from the group consisting of CD28, CD3ε, CD45, CD4, CD5, CD8α, CD9, CD16, CD22, CD33, CD37, CD64, CD80, CD86, CD147, 4-1BB (CD137), CD152, PD-1, CD2, CD27, CD11b, CD11d, CD11a, CD11c, CD84, and CD86.

6: The polypeptide of claim 5, wherein in the transmembrane domain is derived from CD28 or CD8α.

7: The polypeptide of claim 1, wherein the hinge domain and the transmembrane domain are derived from the same protein.

8: The polypeptide of claim 7, wherein the hinge domain and the transmembrane domain are derived from CD28 and comprises the amino acid sequence of SEQ ID NO:431 or 434.

9. (canceled)

10. The polypeptide of claim 8, wherein the intracellular signaling domain is derived from CD32, wherein the intracellular signaling domain comprises the amino acid sequence of SEQ ID NO:544.

11. (canceled)

12: The polypeptide of claim 1, wherein the anti-CD19 CAR comprises a co-stimulatory domain derived from a protein selected from the group consisting of CD27, CD28, CD137 (4-1BB), OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, ligands of CD83, and any combination thereof.

13: The polypeptide of claim 12, wherein the co-stimulatory domain is derived from CD137 (4-1BB) or CD28.

14: The polypeptide of claim 13, wherein the co-stimulatory domain comprises the amino acid sequence of SEQ ID NO:432 or 543.

15: The polypeptide of claim 1, further comprising a signal peptide sequence that comprises a signal peptide, wherein the peptide sequence is connected to the N-terminus of the anti-CD19 CAR.

16: The polypeptide of claim 15, wherein the signal peptide sequence is derived from a CD8α propeptide.

17: The polypeptide of claim 15, wherein the signal peptide sequence comprises the amino acid sequence of SEQ ID NO:515.

18. (canceled)

19: The polypeptide of claim 1, wherein the anti-CD19 CAR comprises the amino acid sequence of any one of SEQ ID NOs:49-51 and 435-437.

20: An isolated nucleic acid encoding the polypeptide of claim 1.

21: An isolated nucleic acid comprising a nucleic acid sequence selected from the group consisting of SEQ ID NO:46-48, 114, 145, 441-458, 482-511, and 570-575.

22-28. (canceled)

29: A lipid nanoparticle (LNP) comprising:
(a) a lipid-immune cell targeting group conjugate comprising the compound of Formula (II): [Lipid]-[optional linker]-[binding domain], wherein the binding domain specifically binds to human CD8α,
(b) an ionizable cationic lipid, and
(c) a nucleic acid encoding the polypeptide of claim 1, wherein the nucleic acid is encapsulated in the LNP.

30: A lipid nanoparticle (LNP) comprising:
(a) a lipid-immune cell targeting group conjugate comprising the compound of Formula (II): [Lipid]-[optional linker]-[binding domain], wherein the binding domain specifically binds to human CD8α,
(b) an ionizable cationic lipid, and
(c) a nucleic acid comprising a nucleic acid sequence selected from the group consisting of SEQ ID NO:46-48, 114, 145, 441-458, 482-511, and 570-575, wherein the nucleic acid is encapsulated in the LNP.

31: The LNP of claim 29, wherein:
a. the binding domain is an immunoglobulin single variable domain (ISVD) that specifically binds to human CD8α, wherein the ISVD comprises a complementarity-determining region 1 (CDR1), a CDR2, and a CDR3 of an ISVD having the sequence selected from the group consisting of SEQ ID NOs: 160-179; or
b. the binding domain is an immunoglobulin single variable domain (ISVD) that specifically binds to human CD8α, wherein the ISVD specifically binding to CD8α comprises a CDR1, a CDR2, and a CDR3 according to the Abm CDR definition,
wherein CDR1 is chosen from the group consisting of:
(i) SEQ ID NO: 244; and
(ii) amino acid sequences that have 3, 2, or 1 amino acid difference with at least one of the amino acid sequences of SEQ ID NO: 244;
wherein CDR2 is chosen from the group consisting of:
(iii) SEQ ID NO: 246; and
(iv) amino acid sequences that have 3, 2, or 1 amino acid difference with at least one of the amino acid sequences of SEQ ID NO: 246; and
wherein CDR3 is chosen from the group consisting of:
(v) SEQ ID NO: 248; and
amino acid sequences that have 3, 2, or 1 amino acid difference with at least one of the amino acid sequences of SEQ ID NO: 248.

32: The LNP of claim 29, wherein the binding domain specifically binding to human CD8α is covalently coupled to the Lipid in Formula (II) via a linker comprising polyethylene glycol (PEG).

33-60. (canceled)

61: The LNP of claim 29, wherein the ionizable cationic lipid comprises a compound of Formula (I): 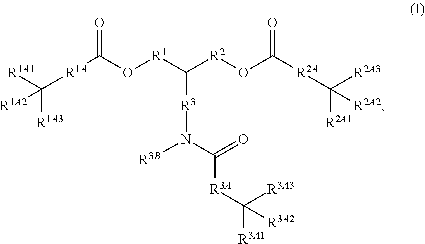
or a salt thereof, or both, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is 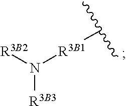
R^{3B1} is C₁₋₆ alkylene; and
R^{3B2} and R^{3B3} are each independently H, unsubstituted C₁₋₆ alkyl, or C₁₋₆ alkyl substituted with 1 or 2-OH.

62-86. (canceled)

87: A method of modulating immune response in a human subject, comprising administering the LNP of claim 29 to the human subject.

88: A method of treating a B cell malignancy in a human subject comprising administering the LNP of claim 29 to the human subject.

89-102. (canceled)


SEQUENCE LISTING (859 sequences)

SEQ ID NO: 1 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAKYSRPDPSEGHVDLDYWGQGTLVTVSS

SEQ ID NO: 2 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAEYSRPDPSEGHVDLDYWGQGTLVTVSS

SEQ ID NO: 3 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAYYSRPDPSEGHVDLDYWGQGTLVTVSS

SEQ ID NO: 4 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAKYSRPDPSEGHVDLDYWGQGTLVTVSS

SEQ ID NO: 5 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAEYSRPDPSEGHVDLDYWGQGTLVTVSS

SEQ ID NO: 6 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAYYSRPDPSEGHVDLDYWGQGTLVTVSS

SEQ ID NO: 7 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAKYSRPDPSEGHVDLDYWGQGTLVTVSS

SEQ ID NO: 8 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAEYSRPDPSEGHVDLDYWGQGTLVTVSS

SEQ ID NO: 9 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAYYSRPDPSEGHVDLDYWGQGTLVTVSS

SEQ ID NO: 10 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAADALPYTVRKYRYWGQGTLVTVSS

SEQ ID NO: 11 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAEDALPYTVRKYRYWGQGTLVTVSS

SEQ ID NO: 12 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAYDALPYTVRKYRYWGQGTLVTVSS

SEQ ID NO: 13 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAADALPYTLRKYRYWGQGTLVTVSS

SEQ ID NO: 14 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAEDALPYTLRKYRYWGQGTLVTVSS

SEQ ID NO: 15 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAYDALPYTLRKYRYWGQGTLVTVSS

SEQ ID NO: 16 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAADALPYTVRKYRYWGQGTLVTVSS

SEQ ID NO: 17 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAADALPYTLRKYRYWGQGTLVTVSS

SEQ ID NO: 18 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAEDALPYTLRKYRYWGQGTLVTVSS

SEQ ID NO: 19 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAYDALPYTLRKYRYWGQGTLVTVSS

SEQ ID NO: 20 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAEDALPYTVRKYRYWGQGTLVTVSS

SEQ ID NO: 21 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAYDALPYTVRKYRYWGQGTLVTVSS

SEQ ID NO: 22 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAADALPYTLRKYRYWGQGTLVTVSS

SEQ ID NO: 23 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAEDALPYTLRKYRYWGQGTLVTVSS

SEQ ID NO: 24 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAYDALPYTLRKYRYWGQGTLVTVSS

SEQ ID NO: 25 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAADALPYTVRKYRYWGQGTLVTVSS

SEQ ID NO: 26 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAEDALPYTVRKYRYWGQGTLVTVSS

SEQ ID NO: 27 - protein, 121 aa, synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAYDALPYTVRKYRYWGQGTLVTVSS

SEQ ID NO: 28 - protein, 135 aa, synthetic construct
EVQLVESGGGLVQAGGSLRLSCAASGFTFDDYAIGWFRQAPGKEREEVSCIRTYDGNTYYIDSVKGRFTISSDNAKNTVYLQMNSLKPEDTAAYYCAAGSYYACAKYSRPDPSENHVDMDYWGKGTLVTVSSGCC

SEQ ID NO: 29 - protein, 135 aa, synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAKYSRPDPSEGHVDLDYWGQGTLVTVSSGGC

SEQ ID NO: 30 - protein, 135 aa, synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAEYSRPDPSEGHVDLDYWGQGTLVTVSSGGC

SEQ ID NO: 31 - protein, 135 aa, synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAYYSRPDPSEGHVDLDYWGQGTLVTVSSGGC

SEQ ID NO: 32 - protein, 135 aa, synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAKYSRPDPSEGHVDLDYWGQGTLVTVSSGGC

SEQ ID NO: 33 - protein, 135 aa, synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAEYSRPDPSEGHVDLDYWGQGTLVTVSSGGC

SEQ ID NO: 34 - protein, 135 aa, synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAYYSRPDPSEGHVDLDYWGQGTLVTVSSGGC

SEQ ID NO: 35 - protein, 135 aa, synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAKYSRPDPSEGHVDLDYWGQGTLVTVSSGGC

SEQ ID NO: 36 - protein, 135 aa, synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAEYSRPDPSEGHVDLDYWGQGTLVTVSSGGC

SEQ ID NO: 37 - protein, 192 aa, synthetic construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITTSFVPVFLPAKPTTTPAPRPPTPAPTIASQPLSLRPEASRPAAGGAVHTRGLDFAGGCGGHHHHHH

SEQ ID NO: 38 - protein, 230 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 39 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 40 - protein, 234 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 41 - protein, 215 aa, synthetic construct; identical to SEQ ID NO: 39

SEQ ID NO: 42 - protein, 227 aa, synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGAGYYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 43 - protein, 214 aa, synthetic construct
DIVMTQSPSSLSASVGDRVTITCRTSRSISQALAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 44 - protein, 135 aa, synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAYYSRPDPSEGHVDLDYWGQGTLVTVSSGGC

SEQ ID NO: 45 - DNA, 112 bp, synthetic construct
  source 1..112: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - DNA, 1110 bp, synthetic construct
  source 1..1110: mol_type = other DNA
atggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcctatttctaagcgactacacgctagcctggttcaggcaggcccccggcaaggagcgcgagtttgtgatcgttagcacgtggaaccgctatacctactacgctgaaggagtgaagggcaggttcacaatttctcgggacaacgccaagaaaaccgtgtatctgcagatgaactccctgcgccccgaggacacagccctgtactactgcgccgcaaaatttcgcatctcatacaacgttagggacgacagcttctacgacctgtggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctgggttcggtccaagcgctcacgcctgctacactcagactacatgaacatgacgccccgcagacctggacctaccaggaaacactatcaaccatatgcccccccaagggactttgcagcatacaggagccgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataa

SEQ ID NO: 47 - DNA, 1110 bp, synthetic construct
  source 1..1110: mol_type = other DNA
atggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcctatttctaagcgactacacgctagcctggttcaggcaggcccccggcaaggagcgcgagtttgtgatcgttagcacgtggaaccgctatacctactacgctgaaggagtgaagggcaggttcacaatttctcgggacaacgccaagaaaaccgtgtatctgcagatgaactccctgcgccccgaggacacagccctgtactactgcgccgcaaaatttcgcatctcatacaacgttagggacgacagcttctacgacctgtggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctggaagcggggacgcaagaagctgctgtatatctttaagcaaccattcatgcggcccgttcaaacgactcaagaagaagacgggtgtagctgccggtttcccgaagaagaagagggcggctgtgaactacgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataa

SEQ ID NO: 48 - DNA, 1122 bp, synthetic construct
  source 1..1122: mol_type = other DNA
atggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcgacaccttcggaacgaaagccatgggatggttcaggcaggcccccggcaagggacgcgagtttgtggccgcaatcacgtggacagcaggcctaacctactacgctgactccgtgaagggcaggttcacaatttctaaagaccgcgccaagaacaccgtggatctgcagatgaactccctgcgccccgaggacacagccctgtactactgcgcctcacggcgcacgttctcatacgagcaccccaattactataccaggtcagcctaccacttttggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctggaagcggggacgcaagaagctgctgtatatctttaagcaaccattcatgcggcccgttcaaacgactcaagaagaagacgggtgtagctgccggtttcccgaagaagaagagggcggctgtgaactacgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataa

SEQ ID NO: 49 - protein, 367 aa, synthetic construct
MALPVTALLLPLALLLHAARPEVQLVESGGGVVQPGGSLRLSCAASGLFLSDYTLAWFRQAPGKEREFVIVSTWNRYTYYAEGVKGRFTISRDNAKKTVYLQMNSLRPEDTALYYCAAKFRISYNVRDDSFYDLWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 367 aa, synthetic construct
MALPVTALLLPLALLLHAARPEVQLVESGGGVVQPGGSLRLSCAASGLFLSDYTLAWFRQAPGKEREFVIVSTWNRYTYYAEGVKGRFTISRDNAKKTVYLQMNSLRPEDTALYYCAAKFRISYNVRDDSFYDLWGQGTLVTVSSAAAIEVMYPPPYLDNEKSNGTIIHVKGKHLCPSPLFPGPSKPFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 371 aa, synthetic construct
MALPVTALLLPLALLLHAARPEVQLVESGGGVVQPGGSLRLSCAASGDTFGTKAMGWFRQAPGKGREFVAAITWTAGLTYYADSVKGRFTISKDRAKNTVDLQMNSLRPEDTALYYCASRRTFSYEHPNYYTRSAYHFWGQGTLVTVSSAAAIEVMYPPPYLDNEKSNGTIIHVKGKHLCPSPLFPGPSKPFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52-113 - skipped (62 entries)

SEQ ID NO: 114 - RNA, 1464 nt, synthetic construct
  source 1..1464: mol_type = other RNA
atggctctcccagtgaccgccctgctgctgcccctagccctccttttgcacgcagcgaggcctgaggtgaagctgcaggagtccggccccgggctggtcgcgcccagccagagcctcagcgtgacctgcaccgtgagcggcgtgagcctgcccgactacggcgtcagctggatccgccagccccccaggaaggggctggagtggctgggcgtgatctggggcagtgagacgacctattacaactccgccctcaagagccgcctaaccatcatcaaggacaacagcaagagccaggtgttcctgaagatgaacagcctgcagaccgacgacaccgccatctactactgcgccaagcactactactacggcgggtcctacgccatggactactgggggcagggcaccagcgtgaccgtcagctcgggcggcggcggcagcgggggaggcgggtcggggggcggcggcagcgacatccagatgacccagaccaccagctccctgagcgcctccctcggcgaccgagtcacgatcagctgcagggcctcccaggacatcagcaagtacctcaactggtaccagcagaagccggacgggaccgtgaagctgctgatctaccacacctcccggctgcacagcggcgtgcccagccggttcagcggctccggcagcggcaccgactacagcctgaccatcagcaacctggagcaggaggacatcgccacctacttctgccagcaggggaacaccctgccctacacgttcggcggcggcaccaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 115-121 - skipped (7 entries)

SEQ ID NO: 122 - protein, 485 aa, synthetic construct
MALPVTALLLPLALLLHAARPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123-144 - skipped (22 entries)

SEQ ID NO: 145 - RNA, 1571 nt, synthetic construct
  source 1..1571: mol_type = other RNA
gggtcaagattagagaacggtcgtagcattatcggaggttctgccaccatggctctcccagtgaccgccctgctgctgcccctagccctccttttgcacgcagcgaggcctgaggtgaagctgcaggagtccggccccgggctggtcgcgcccagccagagcctcagcgtgacctgcaccgtgagcggcgtgagcctgcccgactacggcgtcagctggatccgccagccccccaggaaggggctggagtggctgggcgtgatctggggcagtgagacgacctattacaactccgccctcaagagccgcctaaccatcatcaaggacaacagcaagagccaggtgttcctgaagatgaacagcctgcagaccgacgacaccgccatctactactgcgccaagcactactactacggcgggtcctacgccatggactactgggggcagggcaccagcgtgaccgtcagctcgggcggcggcggcagcgggggaggcgggtcggggggcggcggcagcgacatccagatgacccagaccaccagctccctgagcgcctccctcggcgaccgagtcacgatcagctgcagggcctcccaggacatcagcaagtacctcaactggtaccagcagaagccggacgggaccgtgaagctgctgatctaccacacctcccggctgcacagcggcgtgcccagccggttcagcggctccggcagcggcaccgactacagcctgaccatcagcaacctggagcaggaggacatcgccacctacttctgccagcaggggaacaccctgccctacacgttcggcggcggcaccaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgaggcacatttgaataaattctattaccagttcaagcttggcgtaatcatggtcatagctg

SEQ ID NO: 146-148 - skipped (3 entries)

SEQ ID NO: 149 - protein, 386 aa, Homo sapiens
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 150 - protein, 160 aa, Homo sapiens
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFAC

SEQ ID NO: 151 - protein, 220 aa, synthetic construct
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 6 aa, synthetic construct
HHHHHH

SEQ ID NO: 153 - protein, 396 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGWSHPQFEKGGGSGGGSGGSSAWSHPQFEK

SEQ ID NO: 154 - protein, 29 aa, synthetic construct
WSHPQFEKGGGSGGGSGGSSAWSHPQFEK

SEQ ID NO: 155 - protein, 386 aa, Macaca fascicularis
NQFRVSPLGRTWNLGETVELKCQVLLSNPTSGCSWLFQPRGTAARPTFLLYLSQNKPKAAEGLDTQRFSGKRLGDTFVLTLRDFRQENEGYYFCSALSNSIMYFSHFVPVFLPAKPTTTPAPRPPTPAPTTASQPLSLRPEACRPAAGGSVNTRGLDFACPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 156 - protein, 160 aa, Macaca fascicularis
NQFRVSPLGRTWNLGETVELKCQVLLSNPTSGCSWLFQPRGTAARPTFLLYLSQNKPKAAEGLDTQRFSGKRLGDTFVLTLRDFRQENEGYYFCSALSNSIMYFSHFVPVFLPAKPTTTPAPRPPTPAPTTASQPLSLRPEACRPAAGGSVNTRGLDFAC

SEQ ID NO: 157 - protein, 396 aa, Macaca fascicularis
LQQTPAYIQVQTNKMAMLSCEAKISLGNMRIYWLRQRQAPSSDSHHEFLALWDSTKGTVHGEEVAQEKIAVFRDASRFILNLTSVKPEDSGIYFCMIIGSPELTFGKGTQLSVVDFLPTTAQPTKKSTPKKRGCRLPRPATQKGPLCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGWSHPQFEKGGGSGGGSGGSSAWSHPQFEK

SEQ ID NO: 158 - protein, 147 aa, Macaca fascicularis
LQQTPAYIQVQTNKMAMLSCEAKISLGNMRIYWLRQRQAPSSDSHHEFLALWDSTKGTVHGEEVAQEKIAVFRDASRFILNLTSVKPEDSGIYFCMIIGSPELTFGKGTQLSVVDFLPTTAQPTKKSTPKKRGCRLPRPATQKGPLC

SEQ ID NO: 159 - skipped

SEQ ID NO: 160 - protein, 132 aa, synthetic construct
EVQLVESGGGLVQAGGSLRLSCAASGFTFDDYAIGWFRQAPGKEREEVSCIRTYDGNTYYIDSVKGRFTISSDNAKNTVYLQMNSLKPEDTAAYYCAAGSYYACAKYSRPDPSENHVDMDYWGKGTLVTVSS

SEQ ID NO: 161 - protein, 132 aa, synthetic construct; identical to SEQ ID NO: 1

SEQ ID NO: 162 - protein, 132 aa, synthetic construct; identical to SEQ ID NO: 2

SEQ ID NO: 163 - protein, 132 aa, synthetic construct; identical to SEQ ID NO: 3

SEQ ID NO: 164 - protein, 132 aa, synthetic construct; identical to SEQ ID NO: 4

SEQ ID NO: 165 - protein, 132 aa, synthetic construct; identical to SEQ ID NO: 5

SEQ ID NO: 166 - protein, 132 aa, synthetic construct; identical to SEQ ID NO: 6

SEQ ID NO: 167 - protein, 132 aa, synthetic construct; identical to SEQ ID NO: 7

SEQ ID NO: 168 - protein, 132 aa, synthetic construct; identical to SEQ ID NO: 8

SEQ ID NO: 169 - protein, 132 aa, synthetic construct; identical to SEQ ID NO: 9

SEQ ID NO: 170 - protein, 132 aa, synthetic construct; identical to SEQ ID NO: 160

SEQ ID NO: 171 - protein, 132 aa, synthetic construct; identical to SEQ ID NO: 1

SEQ ID NO: 172 - protein, 132 aa, synthetic construct; identical to SEQ ID NO: 2

SEQ ID NO: 173 - protein, 132 aa, synthetic construct; identical to SEQ ID NO: 3

SEQ ID NO: 174 - protein, 132 aa, synthetic construct; identical to SEQ ID NO: 4

SEQ ID NO: 175 - protein, 132 aa, synthetic construct; identical to SEQ ID NO: 5

SEQ ID NO: 176 - protein, 132 aa, synthetic construct; identical to SEQ ID NO: 6

SEQ ID NO: 177 - protein, 132 aa, synthetic construct; identical to SEQ ID NO: 7

SEQ ID NO: 178 - protein, 132 aa, synthetic construct; identical to SEQ ID NO: 8

SEQ ID NO: 179 - protein, 132 aa, synthetic construct; identical to SEQ ID NO: 9

SEQ ID NO: 180 - protein, 25 aa, synthetic construct
EVQLVESGGGLVQAGGSLRLSCAAS

SEQ ID NO: 181 - protein, 10 aa, synthetic construct
GFTFDDYAIG

SEQ ID NO: 182 - protein, 14 aa, synthetic construct
WFRQAPGKEREEVS

SEQ ID NO: 183 - protein, 10 aa, synthetic construct
CIRTYDGNTY

SEQ ID NO: 184 - protein, 39 aa, synthetic construct
YIDSVKGRFTISSDNAKNTVYLQMNSLKPEDTAAYYCAA

SEQ ID NO: 185 - protein, 23 aa, synthetic construct
GSYYACAKYSRPDPSENHVDMDY

SEQ ID NO: 186 - protein, 11 aa, synthetic construct
WGKGTLVTVSS

SEQ ID NO: 187 - protein, 25 aa, synthetic construct
EVQLVESGGGVVQPGGSLRLSCAAS

SEQ ID NO: 188 - protein, 10 aa, synthetic construct
GFTFEDYAIG

SEQ ID NO: 189 - protein, 14 aa, synthetic construct
WFRQAPGKEREEVS

SEQ ID NO: 190 - protein, 10 aa, synthetic construct
CIRTYDEQTY

SEQ ID NO: 191 - protein, 39 aa, synthetic construct
YADSVKGRFTISRDNAKNTVYLQMNSLRPEDTALYYCAA

SEQ ID NO: 192 - protein, 23 aa, synthetic construct
GSYYACAKYSRPDPSEGHVDLDY

SEQ ID NO: 193 - protein, 11 aa, synthetic construct
WGQGTLVTVSS

SEQ ID NO: 194 - protein, 25 aa, synthetic construct
EVQLVESGGGVVQPGGSLRLSCAAS

SEQ ID NO: 195 - protein, 10 aa, synthetic construct
GFTFEDYAIG

SEQ ID NO: 196 - protein, 14 aa, synthetic construct
WFRQAPGKEREEVS

SEQ ID NO: 197 - protein, 10 aa, synthetic construct
CIRTYDEQTY

SEQ ID NO: 198 - protein, 39 aa, synthetic construct; identical to SEQ ID NO: 191

SEQ ID NO: 199 - protein, 23 aa, synthetic construct
GSYYACAEYSRPDPSEGHVDLDY

SEQ ID NO: 200 - protein, 11 aa, synthetic construct
WGQGTLVTVSS

SEQ ID NO: 201 - protein, 25 aa, synthetic construct
EVQLVESGGGVVQPGGSLRLSCAAS

SEQ ID NO: 202 - protein, 10 aa, synthetic construct
GFTFEDYAIG

SEQ ID NO: 203 - protein, 14 aa, synthetic construct
WFRQAPGKEREEVS

SEQ ID NO: 204 - protein, 10 aa, synthetic construct
CIRTYDEQTY

SEQ ID NO: 205 - protein, 39 aa, synthetic construct; identical to SEQ ID NO: 191

SEQ ID NO: 206 - protein, 23 aa, synthetic construct
GSYYACAYYSRPDPSEGHVDLDY

SEQ ID NO: 207 - protein, 11 aa, synthetic construct
WGQGTLVTVSS

SEQ ID NO: 208 - protein, 25 aa, synthetic construct
EVQLVESGGGVVQPGGSLRLSCAAS

SEQ ID NO: 209 - protein, 10 aa, synthetic construct
GFTFEDYAIG

SEQ ID NO: 210 - protein, 14 aa, synthetic construct
WFRQAPGKEREEVS

SEQ ID NO: 211 - protein, 10 aa, synthetic construct
CIRTYDEQTY

SEQ ID NO: 212 - protein, 39 aa, synthetic construct
YADSVKGRFTISRDNAKNTVDLQMNSLRPEDTALYYCAA

SEQ ID NO: 213 - protein, 23 aa, synthetic construct
GSYYACAKYSRPDPSEGHVDLDY

SEQ ID NO: 214 - protein, 11 aa, synthetic construct
WGQGTLVTVSS

SEQ ID NO: 215 - protein, 25 aa, synthetic construct
EVQLVESGGGVVQPGGSLRLSCAAS

SEQ ID NO: 216 - protein, 10 aa, synthetic construct
GFTFEDYAIG

SEQ ID NO: 217 - protein, 14 aa, synthetic construct
WFRQAPGKEREEVS

SEQ ID NO: 218 - protein, 10 aa, synthetic construct
CIRTYDEQTY

SEQ ID NO: 219 - protein, 39 aa, synthetic construct; identical to SEQ ID NO: 212

SEQ ID NO: 220 - protein, 23 aa, synthetic construct
GSYYACAEYSRPDPSEGHVDLDY

SEQ ID NO: 221 - protein, 11 aa, synthetic construct
WGQGTLVTVSS

SEQ ID NO: 222 - protein, 25 aa, synthetic construct
EVQLVESGGGVVQPGGSLRLSCAAS

SEQ ID NO: 223 - protein, 10 aa, synthetic construct
GFTFEDYAIG

SEQ ID NO: 224 - protein, 14 aa, synthetic construct
WFRQAPGKEREEVS

SEQ ID NO: 225 - protein, 10 aa, synthetic construct
CIRTYDEQTY

SEQ ID NO: 226 - protein, 39 aa, synthetic construct; identical to SEQ ID NO: 212

SEQ ID NO: 227 - protein, 23 aa, synthetic construct
GSYYACAYYSRPDPSEGHVDLDY

SEQ ID NO: 228 - protein, 11 aa, synthetic construct
WGQGTLVTVSS

SEQ ID NO: 229 - protein, 25 aa, synthetic construct
EVQLVESGGGVVQPGGSLRLSCAAS

SEQ ID NO: 230 - protein, 10 aa, synthetic construct
GFTFEDYAIG

SEQ ID NO: 231 - protein, 14 aa, synthetic construct
WFRQAPGKEREEVS

SEQ ID NO: 232 - protein, 10 aa, synthetic construct
CIRTYDEQTY

SEQ ID NO: 233 - protein, 39 aa, synthetic construct
YADSVKGRFTISRDNAKNTVSLQMNSLRPEDTALYYCAA

SEQ ID NO: 234 - protein, 23 aa, synthetic construct
GSYYACAKYSRPDPSEGHVDLDY

SEQ ID NO: 235 - protein, 11 aa, synthetic construct
WGQGTLVTVSS

SEQ ID NO: 236 - protein, 25 aa, synthetic construct
EVQLVESGGGVVQPGGSLRLSCAAS

SEQ ID NO: 237 - protein, 10 aa, synthetic construct
GFTFEDYAIG

SEQ ID NO: 238 - protein, 14 aa, synthetic construct
WFRQAPGKEREEVS

SEQ ID NO: 239 - protein, 10 aa, synthetic construct
CIRTYDEQTY

SEQ ID NO: 240 - protein, 39 aa, synthetic construct; identical to SEQ ID NO: 233

SEQ ID NO: 241 - protein, 23 aa, synthetic construct
GSYYACAEYSRPDPSEGHVDLDY

SEQ ID NO: 242 - protein, 11 aa, synthetic construct
WGQGTLVTVSS

SEQ ID NO: 243 - protein, 25 aa, synthetic construct
EVQLVESGGGVVQPGGSLRLSCAAS

SEQ ID NO: 244 - protein, 10 aa, synthetic construct
GFTFEDYAIG

SEQ ID NO: 245 - protein, 14 aa, synthetic construct
WFRQAPGKEREEVS

SEQ ID NO: 246 - protein, 10 aa, synthetic construct
CIRTYDEQTY

SEQ ID NO: 247 - protein, 39 aa, synthetic construct; identical to SEQ ID NO: 233

SEQ ID NO: 248 - protein, 23 aa, synthetic construct
GSYYACAYYSRPDPSEGHVDLDY

SEQ ID NO: 249 - protein, 11 aa, synthetic construct
WGQGTLVTVSS

SEQ ID NO: 250 - protein, 30 aa, synthetic construct
EVQLVESGGGLVQAGGSLRLSCAASGFTFD

SEQ ID NO: 251 - protein, 5 aa, synthetic construct
DYAIG

SEQ ID NO: 252 - protein, 14 aa, synthetic construct
WFRQAPGKEREEVS

SEQ ID NO: 253 - protein, 17 aa, synthetic construct
CIRTYDGNTYYIDSVKG

SEQ ID NO: 254 - protein, 32 aa, synthetic construct
RFTISSDNAKNTVYLQMNSLKPEDTAAYYCAA

SEQ ID NO: 255 - protein, 23 aa, synthetic construct
GSYYACAKYSRPDPSENHVDMDY

SEQ ID NO: 256 - protein, 11 aa, synthetic construct
WGKGTLVTVSS

SEQ ID NO: 257 - protein, 30 aa, synthetic construct
EVQLVESGGGVVQPGGSLRLSCAASGFTFE

SEQ ID NO: 258 - protein, 5 aa, synthetic construct
DYAIG

SEQ ID NO: 259 - protein, 14 aa, synthetic construct
WFRQAPGKEREEVS

SEQ ID NO: 260 - protein, 17 aa, synthetic construct
CIRTYDEQTYYADSVKG

SEQ ID NO: 261 - protein, 32 aa, synthetic construct
RFTISRDNAKNTVYLQMNSLRPEDTALYYCAA

SEQ ID NO: 262 - protein, 23 aa, synthetic construct
GSYYACAKYSRPDPSEGHVDLDY

SEQ ID NO: 263 - protein, 11 aa, synthetic construct
WGQGTLVTVSS

SEQ ID NO: 264 - protein, 30 aa, synthetic construct; identical to SEQ ID NO: 257

SEQ ID NO: 265 - protein, 5 aa, synthetic construct
DYAIG

SEQ ID NO: 266 - protein, 14 aa, synthetic construct
WFRQAPGKEREEVS

SEQ ID NO: 267 - protein, 17 aa, synthetic construct
CIRTYDEQTYYADSVKG

SEQ ID NO: 268 - protein, 32 aa, synthetic construct; identical to SEQ ID NO: 261

SEQ ID NO: 269 - protein, 23 aa, synthetic construct
GSYYACAEYSRPDPSEGHVDLDY

SEQ ID NO: 270 - protein, 11 aa, synthetic construct
WGQGTLVTVSS

SEQ ID NO: 271 - protein, 30 aa, synthetic construct; identical to SEQ ID NO: 257

SEQ ID NO: 272 - protein, 5 aa, synthetic construct
DYAIG

SEQ ID NO: 273 - protein, 14 aa, synthetic construct
WFRQAPGKEREEVS

SEQ ID NO: 274 - protein, 17 aa, synthetic construct
CIRTYDEQTYYADSVKG

SEQ ID NO: 275 - protein, 32 aa, synthetic construct; identical to SEQ ID NO: 261

SEQ ID NO: 276 - protein, 23 aa, synthetic construct
GSYYACAYYSRPDPSEGHVDLDY

SEQ ID NO: 277 - protein, 11 aa, synthetic construct
WGQGTLVTVSS

SEQ ID NO: 278 - protein, 30 aa, synthetic construct; identical to SEQ ID NO: 257

SEQ ID NO: 279 - protein, 5 aa, synthetic construct
DYAIG

SEQ ID NO: 280 - protein, 14 aa, synthetic construct
WFRQAPGKEREEVS

SEQ ID NO: 281 - protein, 17 aa, synthetic construct
CIRTYDEQTYYADSVKG

SEQ ID NO: 282 - protein, 32 aa, synthetic construct
RFTISRDNAKNTVDLQMNSLRPEDTALYYCAA

SEQ ID NO: 283 - protein, 23 aa, synthetic construct
GSYYACAKYSRPDPSEGHVDLDY

SEQ ID NO: 284 - protein, 11 aa, synthetic construct
WGQGTLVTVSS

SEQ ID NO: 285 - protein, 30 aa, synthetic construct; identical to SEQ ID NO: 257

SEQ ID NO: 286 - protein, 5 aa, synthetic construct
DYAIG

SEQ ID NO: 287 - protein, 14 aa, synthetic construct
WFRQAPGKEREEVS

SEQ ID NO: 288 - protein, 17 aa, synthetic construct
CIRTYDEQTYYADSVKG

SEQ ID NO: 289 - protein, 32 aa, synthetic construct; identical to SEQ ID NO: 282

SEQ ID NO: 290 - protein, 23 aa, synthetic construct
GSYYACAEYSRPDPSEGHVDLDY

SEQ ID NO: 291 - protein, 11 aa, synthetic construct
WGQGTLVTVSS

SEQ ID NO: 292 - protein, 30 aa, synthetic construct; identical to SEQ ID NO: 257

SEQ ID NO: 293 - protein, 5 aa, synthetic construct
DYAIG

SEQ ID NO: 294 - protein, 14 aa, synthetic construct
WFRQAPGKEREEVS

SEQ ID NO: 295 - protein, 17 aa, synthetic construct
CIRTYDEQTYYADSVKG

SEQ ID NO: 296 - protein, 32 aa, synthetic construct; identical to SEQ ID NO: 282

SEQ ID NO: 297 - protein, 23 aa, synthetic construct
GSYYACAYYSRPDPSEGHVDLDY

SEQ ID NO: 298 - protein, 11 aa, synthetic construct
WGQGTLVTVSS

SEQ ID NO: 299 - protein, 30 aa, synthetic construct; identical to SEQ ID NO: 257

SEQ ID NO: 300 - protein, 5 aa, synthetic construct
DYAIG

SEQ ID NO: 301 - protein, 14 aa, synthetic construct
WFRQAPGKEREEVS

SEQ ID NO: 302 - protein, 17 aa, synthetic construct
CIRTYDEQTYYADSVKG

SEQ ID NO: 303 - protein, 32 aa, synthetic construct
RFTISRDNAKNTVSLQMNSLRPEDTALYYCAA

SEQ ID NO: 304 - protein, 23 aa, synthetic construct
GSYYACAKYSRPDPSEGHVDLDY

SEQ ID NO: 305 - protein, 11 aa, synthetic construct
WGQGTLVTVSS

SEQ ID NO: 306 - protein, 30 aa, synthetic construct; identical to SEQ ID NO: 257

SEQ ID NO: 307 - protein, 5 aa, synthetic construct
DYAIG

SEQ ID NO: 308 - protein, 14 aa, synthetic construct
WFRQAPGKEREEVS

SEQ ID NO: 309 - protein, 17 aa, synthetic construct
CIRTYDEQTYYADSVKG

SEQ ID NO: 310 - protein, 32 aa, synthetic construct; identical to SEQ ID NO: 303

SEQ ID NO: 311 - protein, 23 aa, synthetic construct
GSYYACAEYSRPDPSEGHVDLDY

SEQ ID NO: 312 - protein, 11 aa, synthetic construct
WGQGTLVTVSS

SEQ ID NO: 313 - protein, 30 aa, synthetic construct; identical to SEQ ID NO: 257

SEQ ID NO: 314 - protein, 5 aa, synthetic construct
DYAIG

SEQ ID NO: 315 - protein, 14 aa, synthetic construct
WFRQAPGKEREEVS

SEQ ID NO: 316 - protein, 17 aa, synthetic construct
YADSVKGCIRTYDEQTY

SEQ ID NO: 317 - protein, 32 aa, synthetic construct; identical to SEQ ID NO: 303

SEQ ID NO: 318 - protein, 23 aa, synthetic construct
GSYYACAYYSRPDPSEGHVDLDY

SEQ ID NO: 319 - protein, 11 aa, synthetic construct
WGQGTLVTVSS

SEQ ID NO: 320 - protein, 115 aa, synthetic construct [sequence omitted]

SEQ ID NO: 321 - protein, 115 aa, synthetic construct [sequence omitted]

SEQ ID NO: 322 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 323 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 324 - protein, 115 aa, synthetic construct [sequence omitted]

SEQ ID NO: 325 - protein, 116 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 326 - protein, 115 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 327 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 328 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 329 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 330 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 331 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 332 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 333 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 334 - protein, 123 aa, synthetic construct
EVQLVESGGGVVQPGGSLRLSCAASGLTFSSYAMGWFRQAPGKERERVVSISRGGGYTYYADSVKGRFTISRDNSENTVYLQMNSLRPEDTALYYCAAARYWATGSEYEFDYWGQGTLVTVSS

SEQ ID NO: 335 - protein, 115 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 336 - skipped

SEQ ID NO: 337 - protein, 5 aa, synthetic construct
GGGGS

SEQ ID NO: 338 - protein, 7 aa, synthetic construct
SGGSGGS

SEQ ID NO: 339 - protein, 8 aa, synthetic construct
GGGGSGGS

SEQ ID NO: 340 - protein, 9 aa, synthetic construct
GGGGSGGGS

SEQ ID NO: 341 - protein, 10 aa, synthetic construct
GGGGSGGGGS

SEQ ID NO: 342 - protein, 15 aa, synthetic construct
GGGGSGGGGSGGGGS

SEQ ID NO: 343 - protein, 18 aa, synthetic construct
GGGGSGGGGSGGGGSGGS

SEQ ID NO: 344 - protein, 20 aa, synthetic construct
GGGGSGGGGSGGGGSGGGGS

SEQ ID NO: 345 - protein, 25 aa, synthetic construct
GGGGSGGGGSGGGGSGGGGSGGGGS

SEQ ID NO: 346 - protein, 30 aa, synthetic construct
GGGGSGGGGSGGGGSGGGGSGGGGSGGGGS

SEQ ID NO: 347 - protein, 35 aa, synthetic construct
GGGGSGGGGSGGGGSGGGGSGGGGSGGGGSGGGGS

SEQ ID NO: 348 - protein, 40 aa, synthetic construct
GGGGSGGGGSGGGGSGGGGSGGGGSGGGGSGGGGSGGGGS

SEQ ID NO: 349 - protein, 15 aa, synthetic construct
EPKSCDKTHTCPPCP

SEQ ID NO: 350 - protein, 24 aa, synthetic construct
GGGGSGGGSEPKSCDKTHTCPPCP

SEQ ID NO: 351 - protein, 12 aa, synthetic construct
EPKTPKPQPAAA

SEQ ID NO: 352 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 353 - protein, 6 aa, synthetic construct
  VARIANT 6: note = Can be present in repeats of 1 to 10
VTVSSX

SEQ ID NO: 354 - protein, 5 aa, synthetic construct
VKVSS

SEQ ID NO: 355 - protein, 5 aa, synthetic construct
VQVSS

SEQ ID NO: 356 - protein, 5 aa, synthetic construct
VTVKS

SEQ ID NO: 357 - protein, 5 aa, synthetic construct
VTVQS

SEQ ID NO: 358 - protein, 5 aa, synthetic construct
VKVKS

SEQ ID NO: 359 - protein, 5 aa, synthetic construct
VKVQS

SEQ ID NO: 360 - protein, 5 aa, synthetic construct
VQVKS

SEQ ID NO: 361 - protein, 5 aa, synthetic construct
VQVQS

SEQ ID NO: 362 - protein, 6 aa, synthetic construct
VTVSSA

SEQ ID NO: 363 - protein, 6 aa, synthetic construct
VKVSSA

SEQ ID NO: 364 - protein, 6 aa, synthetic construct
VQVSSA

SEQ ID NO: 365 - protein, 6 aa, synthetic construct
VTVKSA

SEQ ID NO: 366 - protein, 6 aa, synthetic construct
VTVQSA

SEQ ID NO: 367 - protein, 6 aa, synthetic construct
VKVKSA

SEQ ID NO: 368 - protein, 6 aa, synthetic construct
VKVQSA

SEQ ID NO: 369 - protein, 6 aa, synthetic construct
VQVKSA

SEQ ID NO: 370 - protein, 6 aa, synthetic construct
VQVQSA

SEQ ID NO: 371 - protein, 5 aa, synthetic construct
VTVSS

SEQ ID NO: 372 - protein, 4 aa, synthetic construct
KERE

SEQ ID NO: 373 - protein, 4 aa, synthetic construct
KQRE

SEQ ID NO: 374 - protein, 4 aa, synthetic construct
GLEW

SEQ ID NO: 375 - protein, 5 aa, synthetic construct
KEREL

SEQ ID NO: 376 - protein, 5 aa, synthetic construct
KEREF

SEQ ID NO: 377 - protein, 5 aa, synthetic construct
KQREL

SEQ ID NO: 378 - protein, 5 aa, synthetic construct
KQREF

SEQ ID NO: 379 - protein, 5 aa, synthetic construct
KEREG

SEQ ID NO: 380 - protein, 5 aa, synthetic construct
KQREW

SEQ ID NO: 381 - protein, 5 aa, synthetic construct
KQREG

SEQ ID NO: 382 - protein, 4 aa, synthetic construct
TERE

SEQ ID NO: 383 - protein, 5 aa, synthetic construct
TEREL

SEQ ID NO: 384 - protein, 4 aa, synthetic construct
TQRE

SEQ ID NO: 385 - protein, 5 aa, synthetic construct
TQREL

SEQ ID NO: 386 - protein, 4 aa, synthetic construct
KECE

SEQ ID NO: 387 - protein, 5 aa, synthetic construct
KECEL

SEQ ID NO: 388 - protein, 5 aa, synthetic construct
KECER

SEQ ID NO: 389 - protein, 4 aa, synthetic construct
KQCE

SEQ ID NO: 390 - protein, 5 aa, synthetic construct
KQCEL

SEQ ID NO: 391 - protein, 4 aa, synthetic construct
RERE

SEQ ID NO: 392 - protein, 5 aa, synthetic construct
REREG

SEQ ID NO: 393 - protein, 4 aa, synthetic construct
RQRE

SEQ ID NO: 394 - protein, 5 aa, synthetic construct
RQREL

SEQ ID NO: 395 - protein, 5 aa, synthetic construct
RQREF

SEQ ID NO: 396 - protein, 5 aa, synthetic construct
RQREW

SEQ ID NO: 397 - protein, 4 aa, synthetic construct
QERE

SEQ ID NO: 398 - protein, 5 aa, synthetic construct
QEREG

SEQ ID NO: 399 - protein, 4 aa, synthetic construct
QQRE

SEQ ID NO: 400 - protein, 5 aa, synthetic construct
QQREW

SEQ ID NO: 401 - protein, 5 aa, synthetic construct
QQREL

SEQ ID NO: 402 - protein, 5 aa, synthetic construct
QQREF

SEQ ID NO: 403 - protein, 4 aa, synthetic construct
KGRE

SEQ ID NO: 404 - protein, 5 aa, synthetic construct
KGREG

SEQ ID NO: 405 - protein, 4 aa, synthetic construct
KDRE

SEQ ID NO: 406 - protein, 5 aa, synthetic construct
KDREV

SEQ ID NO: 407 - protein, 5 aa, synthetic construct
DECKL

SEQ ID NO: 408 - protein, 5 aa, synthetic construct
NVCEL

SEQ ID NO: 409 - protein, 4 aa, synthetic construct
GVEW

SEQ ID NO: 410 - protein, 4 aa, synthetic construct
EPEW

SEQ ID NO: 411 - protein, 4 aa, synthetic construct
GLER

SEQ ID NO: 412 - protein, 4 aa, synthetic construct
DQEW

SEQ ID NO: 413 - protein, 4 aa, synthetic construct
DLEW

SEQ ID NO: 414 - protein, 4 aa, synthetic construct
GIEW

SEQ ID NO: 415 - protein, 4 aa, synthetic construct
ELEW

SEQ ID NO: 416 - protein, 4 aa, synthetic construct
GPEW

SEQ ID NO: 417 - protein, 4 aa, synthetic construct
EWLP

SEQ ID NO: 418 - protein, 4 aa, synthetic construct
GPER

SEQ ID NO: 419 - protein, 132 aa, synthetic construct
EVQLVESGGGLVQAGGSLRLSCAASGSTFSDYGVGWFRQAPGKGREFVADIDWNGEHTSYADSVKGRFATSRDNAKNTAYLQMNSLKPEDTAVYYCAADALPYTVRKYNYWGQGTQVTVSSGGCGGHHHHHH

SEQ ID NO: 420 - protein, 25 aa, synthetic construct
EVQLVESGGGLVQAGGSLRLSCAAS

SEQ ID NO: 421 - protein, 8 aa, synthetic construct
GSTFSDYG

SEQ ID NO: 422 - protein, 17 aa, synthetic construct
VGWFRQAPGKGREFVAD

SEQ ID NO: 423 - protein, 8 aa, synthetic construct
IDWNGEHT

SEQ ID NO: 424 - protein, 38 aa, synthetic construct
SYADSVKGRFATSRDNAKNTAYLQMNSLKPEDTAVYYC

SEQ ID NO: 425 - protein, 14 aa, synthetic construct
AADALPYTVRKYNY

SEQ ID NO: 426 - protein, 22 aa, synthetic construct
WGQGTQVTVSSGGCGGHHHHHH

SEQ ID NO: 427 - protein, 5 aa, synthetic construct
DYTLA

SEQ ID NO: 428 - protein, 16 aa, synthetic construct
VSTWNRYTYYAEGVKG

SEQ ID NO: 429 - protein, 16 aa, synthetic construct
KFRISYNVRDDSFYDL

SEQ ID NO: 430 - protein, 17 aa, synthetic construct
MWLQNLLLLGAVVCSIS

SEQ ID NO: 431 - protein, 66 aa, synthetic construct
IEVMYPPPYLDNEKSNGTIIHVKGKHLCPSPLFPGPSKPFWVLVVVGGVLACYSLLVTVAFIIFWV

SEQ ID NO: 432 - protein, 41 aa, synthetic construct
RGRKKLLYIFKQPFMRPVQTTQEEDGCSCRFPEEEEGGCEL

SEQ ID NO: 433 - protein, 124 aa, synthetic construct
EVQLVESGGGVVQPGGSLRLSCAASGLFLSDYTLAWFRQAPGKEREFVIVSTWNRYTYYAEGVKGRFTISRDNAKKTVYLQMNSLRPEDTALYYCAAKFRISYNVRDDSFYDLWGQGTLVTVSS

SEQ ID NO: 434 - protein, 66 aa, synthetic construct
IEVMYPPPYLDNEKSNGTIIHVKGKHLCPSPLFPGPSKPFWVLVVVGGVLACYSLLVTVAFIIFWK

SEQ ID NO: 435 - protein, 346 aa, synthetic construct
EVQLVESGGGVVQPGGSLRLSCAASGLFLSDYTLAWFRQAPGKEREFVIVSTWNRYTYYAEGVKGRFTISRDNAKKTVYLQMNSLRPEDTALYYCAAKFRISYNVRDDSFYDLWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 436 - protein, 346 aa, synthetic construct
EVQLVESGGGVVQPGGSLRLSCAASGLFLSDYTLAWFRQAPGKEREFVIVSTWNRYTYYAEGVKGRFTISRDNAKKTVYLQMNSLRPEDTALYYCAAKFRISYNVRDDSFYDLWGQGTLVTVSSAAAIEVMYPPPYLDNEKSNGTIIHVKGKHLCPSPLFPGPSKPFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 437 - protein, 350 aa, synthetic construct
EVQLVESGGGVVQPGGSLRLSCAASGDTFGTKAMGWFRQAPGKGREFVAAITWTAGLTYYADSVKGRFTISKDRAKNTVDLQMNSLRPEDTALYYCASRRTFSYEHPNYYTRSAYHFWGQGTLVTVSSAAAIEVMYPPPYLDNEKSNGTIIHVKGKHLCPSPLFPGPSKPFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 438 - protein, 40 aa, synthetic construct
IEVMYPPPYLDNEKSNGTIIHVKGKHLCPSPLFPGPSKPF

SEQ ID NO: 439 - protein, 40 aa, synthetic construct; identical to SEQ ID NO: 438

SEQ ID NO: 440 - protein, 26 aa, synthetic construct
WVLVVVGGVLACYSLLVTVAFIIFWK

SEQ ID NO: 441 - DNA, 1461 bp, synthetic construct
  source 1..1461: mol_type = other DNA
atggagaccgacaccctcctgctgtgggtgctgctgctttgggtgcccgggagcacgggggaggtgaagctgcaggagtccggccccgggctggtcgcgcccagccagagcctcagcgtgacctgcaccgtgagcggcgtgagcctgcccgactacggcgtcagctggatccgccagccccccaggaaggggctggagtggctgggcgtgatctggggcagtgagacgacctattacaactccgccctcaagagccgcctaaccatcatcaaggacaacagcaagagccaggtgttcctgaagatgaacagcctgcagaccgacgacaccgccatctactactgcgccaagcactactactacggcgggtcctacgccatggactactgggggcagggcaccagcgtgaccgtcagctcgggcggcggcggcagcgggggaggcgggtcggggggcggcggcagcgacatccagatgacccagaccaccagctccctgagcgcctccctcggcgaccgagtcacgatcagctgcagggcctcccaggacatcagcaagtacctcaactggtaccagcagaagccggacgggaccgtgaagctgctgatctaccacacctcccggctgcacagcggcgtgcccagccggttcagcggctccggcagcggcaccgactacagcctgaccatcagcaacctggagcaggaggacatcgccacctacttctgccagcaggggaacaccctgccctacacgttcggcggcggcaccaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 442 - DNA, 1482 bp, synthetic construct
  source 1..1482: mol_type = other DNA
atggagaccgacaccctcctgctgtgggtgctgctgctttgggtgcccgggagcacgggggaggtgaagctgcaggagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcggcggcggcagcggcggcggcggcagcggcggcggcggcagcgacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagatcaaggcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacaagcgcggcaggaagaagctcctgtacatcttcaagcagccgttcatgcgcccggtgcagacgacccaggaggaggacgggtgcagctgccggttcccggaggaggaggagggcggctgcgagctgagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 443 - DNA, 1434 bp, synthetic construct
  source 1..1434: mol_type = other DNA
atggagaccgacaccctcctgctgtgggtgctgctgctttgggtgcccgggagcacgggccaggtgcagctggtggagagcggcggcggagtggtccagcccggacgcagcctgcgactgagctgcgcggcaagcgggttcaccttcagcagctacgggatgcactgggtccgtcaggcccccggcaagggcctcgagtgggtggccgtcatctggtacgacgggagcaacaagtactacgtggacagcgtgaagggccggttcacgatcagcagggacaactccaagaacacgatgtacctgcagatgaactccctgcgggccgaggacaccgccgtgtattactgcgccaggcgaggctacaactggaacatcgccttcgacatctggggccagggcaccatggtgacagtgtcgagcggaggcggcggctccggaggcggaggcagcggcggcggcggatcgggaggaggcggctccgacatccagatgacccagagcccaagcaccctaagcgccagcgtgggcgaccgggtgaccatcacctgcagggcctcccagagcatcagctcctggctggcctggtaccagcagaagccgggcaaggcgcccaagctgctcatctacaaggcctcgagcctggagaacggcgtgccctcccgcttcagcggctccggcagcggaaccgagttcaccctcacaatcagctccctgcaacccgacgacttcgcgacctactactgccaacagtacaacagctactggaccttcggccagggcaccaaggtcgagatcaaggcaagcagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 444 - DNA, 1515 bp, synthetic construct
  source 1..1515: mol_type = other DNA
atggagaccgacaccctcctgctgtgggtgctgctgctttgggtgcccgggagcacgggccaggtccagctcgtgcagagcggggcggaggtgaagaagcccggggcatcggtgaaggtgagctgtaaggccagcggctacacgttcaccagctacggcatcagctgggtgcgccaggcccccggccagggcctagagtggatggggtggatcagcgcatacaacggcaacaccaattacgcccagaaactgcaggaccgcgtgaccatgaccacccacacctccaccagcgccgcatacatggagctgaggagcctgaggtccgacgacaccgccgtgtactactgtgcccgagacggcggcctggtcgccgcctccccggactactggggccaggggaccctggtgacagtgtccagtggaggcggcggctccggaggcggaggcagcggcggcggcggatcgggaggaggcggctccgacatcgtgatgacacagagccccgacagcctggcggtcagcctgggagagagggccacaatcaactgcaagagcagccagagtgtcctctacaacagcaacaacaagaactatctcgcctggtaccagcagaagcccggacagccccccaagctgctgatctaccgtgccagcacccgggacagcggcgtgcccgaccggttcagcggcagtggcagcggcacggacttcaccctgaccatctcgagcctccaagccgaggacgtggccctgtactactgccagcaatactactccaccccctacacgtttggccagggcaccaagctggagatcaaagcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 445 - DNA, 1185 bp, synthetic construct
  source 1..1185: mol_type = other DNA
atggagaccgacaccctcctgctgtgggtgctgctgctttgggtgcccgggagcacgggcgaggtgcagctggtcgagtccggcggcggcctggtgcaggccggcgagagcctgcggctctcgtgcatcgccagcggccgcacgatgagtagctacgccatggcctggttcagacaggcgcccggcaaggagagagagttcgtggcggtgagctccctgtccggcgggaccagcgactacgccgacagcgtgaaggggcgtttcatcatcagcagggacaacgccaggagcaccgtgaccctccagatgaacaacctgaagcccgaggacaccgccgtgtactactgcgcagcccgcccggtctacatccgatactccatccgggactatgacatctggggacagggcaccctggtcaccgtgagcagcgccagccttagcaacagcatcatgtacttcagccactttgtcccggtcttcctgccagccaagccgaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 446 - DNA, 1125 bp, synthetic construct
  source 1..1125: mol_type = other DNA
atggagaccgacaccctcctgctgtgggtgctgctgctttgggtgcccgggagcacgggcgaggtgcaactggtcgagagcggcggcggactggtgcaggcaggcgagagcctgaggctgtcctgcatcgccagcggccgaaccatgagcagctacgccatggcgtggttcagacaggcaccgggcaaggagcgcgagttcgtggccgtgagctccctgagcggcgggaccagcgactacgccgacagcgtgaagggccggttcatcatcagccgcgacaacgccaggtccaccgtgaccctccagatgaacaacctgaagcccgaggacacggcagtgtactactgcgcggccaggcccgtctacatccggtactcgatccgtgactacgacatctggggccagggcaccctcgtcaccgtgtccagcgcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 447 - DNA, 1185 bp, synthetic construct
  source 1..1185: mol_type = other DNA
atggagaccgacaccctcctgctgtgggtgctgctgctttgggtgcccgggagcacgggcgaggtgcagctggtcgagagcggcggaggcctggtgcaggccggcggcagcctgcgtctgagctgcgccttcagcgggcggaccttctcgtcctacgccatgggctggttccgccaggcgccgggcaaggagcgagagtttgtcgcaacgataagcctctccggcggcaccacgtggtacgccgacagcgtgaagggccggtttaccatcagcagggacaacggagcgtccaccgcctacctgcagatgaacagcctcaagcccgaggataccgcagtatactactgcgcggcaaggcccgtctaccacaagaccagcgcccgcgactacgacttctggggccagggcacccaggtgaccgtgagcagtgccagccttagcaacagcatcatgtacttcagccactttgtcccggtcttcctgccagccaagccgaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 448 - DNA, 1125 bp, synthetic construct
  source 1..1125: mol_type = other DNA
atggagaccgacaccctcctgctgtgggtgctgctgctttgggtgcccgggagcacgggcgaggtccagctcgtcgagagcggcggcggcctggtgcaggcgggcggcagcctgaggctgagttgcgcgttctccggccggacctttagcagctacgccatgggctggttcaggcaggccccgggcaaggaacgcgagttcgtggccaccatctcactgagcggcgggacgacctggtacgccgactccgtgaagggccgcttcaccatcagcagagacaacggcgccagcaccgcatacctgcagatgaacagcctcaagcccgaggacacagccgtgtactactgcgccgcacggcccgtgtaccacaagacctccgcccgagactacgacttctggggacaggggacccaggtcacggtgagttcggcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 449 - DNA, 1185 bp, synthetic construct
  source 1..1185: mol_type = other DNA
atggagaccgacaccctcctgctgtgggtgctgctgctttgggtgcccgggagcacgggcgaggtgcagctggtggagagcggcggcggcctggtgcaggccggcggcagcctgcgtctcagctgcgcggcctcggggttcaccttcagcagctacgccatggcctggttccggcaggccccgggcaaggagcgagagtttgtggcggtcatcagcctgagcggcggctccgtcgattaccgggacagcgtcaagggccgcttcacgatctccagggacaacgcacagagcaccgtgtccctccagatgaacaacctgaagcccgaggacaccgccatatattactgcgccgcccgcccggtgtacatgaagtggagtgagagggacttcgactactggggccagggcacactggtgaccgtgagctccgccagccttagcaacagcatcatgtacttcagccactttgtcccggtcttcctgccagccaagccgaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 450 - DNA, 1125 bp, synthetic construct
  source 1..1125: mol_type = other DNA
atggagaccgacaccctcctgctgtgggtgctgctgctttgggtgcccgggagcacgggcgaggtccagctggtggagtccggcggcgggctcgtgcaggccggcggcagcctgaggctgagttgcgccgcgagcgggttcaccttcagcagctacgccatggcctggttcaggcaggcgccgggcaaggagcgcgagttcgtggccgtcatcagcctgtccggcggcagcgtcgattaccgggacagcgtaaagggccgcttcacaatctcccgagacaacgcccagagcacagtgtccctgcagatgaacaacctcaagcccgaggacacggccatctactactgcgccgcccggcccgtgtacatgaagtggagtgagcgtgacttcgactactgggggcagggcactctggtgaccgtgagctcggcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 451 - DNA, 1101 bp, synthetic construct
  source 1..1101: mol_type = other DNA
atggagaccgacaccctcctgctgtgggtgctgctgctttgggtgcccgggagcacgggcgaggtccagctggtggagtccggcggcggcctggtgcaggccggagggagcctgaagctggcgtgcgccgcctccgtgcacaccagctacacgtacggcatgggctggttcaggcaggcgcccggcaaggagcgcgagttcgtcgccgtgatcagctcggacggcgtccgaaccaactacgcggacagcgtgcagggccggttcaccatcagcagggacaacgccaagaacaccatctacctccagatgaacaacctgaagaccgaggacagcgccatctactactgcgccgccgggagcggcctcttctccgcaggctacgagtatcagtattggggccagggcacccaggtgaccgtgagcagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 452 - DNA, 1122 bp, synthetic construct
  source 1..1122: mol_type = other DNA
atggagaccgacaccctcctgctgtgggtgctgctgctttgggtgcccgggagcacgggcgaggtgcagctggtggagtccggcggcggcctggtccaggccggcggctcactgaagctggcctgcgcggcgagcgtccacacctcgtacacctacggcatgggctggttcaggcaggcgcccgggaaggagcgggagttcgtggccgtgatcagtagcgacggcgtgcgcaccaactacgccgacagcgtgcagggccgattcaccataagcagggacaacgccaagaacaccatctacctccagatgaacaacctcaagacggaggacagcgccatctactattgcgccgccggctccgggctgttctccgccggctacgagtaccagtactggggccagggcacccaggtcaccgtgagcagcgcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 453 - DNA, 1188 bp, synthetic construct
  source 1..1188: mol_type = other DNA
atggagaccgacaccctcctgctgtgggtgctgctgctttgggtgcccgggagcacgggcgaggtgcagctggtggagagcggaggcggcctggtgcaggcggagggcagcctccggctgagctgcacggccagcggactgacctttgacaactacgccatgggctggttccgacaggccccgggcaaggagagggagttcgtggccaccatctccgccgcgggccacagaaccatcttcaccaacagcgtgcgcgggaggtttaccgcctcgcgggacaacgccaagaagaccacgtacctgcagatgaatagcctgaagccggaggacaccgccgtctactactgcgccgcctccccaagccagtacgggagcatgggcaagtccgcctacgactattggggccagggcaccctcgtgaccgtcagcagcgccagccttagcaacagcatcatgtacttcagccactttgtcccggtcttcctgccagccaagccgaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 454 - DNA, 1128 bp, synthetic construct
  source 1..1128: mol_type = other DNA
atggagaccgacaccctcctgctgtgggtgctgctgctttgggtgcccgggagcacgggcgaggtccaactggtggagagcggaggcggactcgtgcaggccgagggctccctgcggctgagctgcaccgcatccggcctcacattcgacaactacgccatgggctggttccgccaggccccaggcaaggaaagggagttcgtggcgacgatcagcgcggccggccaccgcaccatcttcaccaacagcgtgcgcggaaggttcaccgccagccgagacaacgccaagaagaccacgtacctgcagatgaactccctgaagcccgaggacaccgccgtgtactactgtgccgccagcccgagccagtacgggagcatgggcaagagcgcctacgactactggggccagggcaccctggtcaccgtgtcgagcgcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 455 - DNA, 1524 bp, synthetic construct
  source 1..1524: mol_type = other DNA
atggagaccgacaccctcctgctgtgggtgctgctgctttgggtgcccgggagcacgggcgaggtccaactggtggagagcggaggcggactcgtgcaggccgagggctccctgcggctgagctgcaccgcatccggcctcacattcgacaactacgccatgggctggttccgccaggccccaggcaaggaaagggagttcgtggcgacgatcagcgcggccggccaccgcaccatcttcaccaacagcgtgcgcggaaggttcaccgccagccgagacaacgccaagaagaccacgtacctgcagatgaactccctgaagcccgaggacaccgccgtgtactactgtgccgccagcccgagccagtacgggagcatgggcaagagcgcctacgactactggggccagggcaccctggtcaccgtgtcgagcggaggcggcggctccggaggcggaggcagcggcggcggcggatcggaggtgcagctggtggagtccggcggcgggctggtgcaggccggcgacagcctgaggctctcgtgcgccgccagcggcaggaccctgagcagctacacgaccggctggttccgtcaggggctgggcaaggagcgcgagttcgtggccgcgatcaccaagaacggcccctacctctactacaaggacagcgtcaggggccgcttcaccatctcccgagacagcgccaagaccaccgtgttcctgcagatggacaacctgaagccggaggacaccgccgtctattactgcgccgtaagcgacgcccagtacgacatccggaaccgggagaagtactactactggggccagggcaccctggtgacggtctccagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 456 - DNA, 1536 bp, synthetic construct
  source 1..1536: mol_type = other DNA
atggagaccgacaccctcctgctgtgggtgctgctgctttgggtgcccgggagcacgggcgaggtccaactggtggagagcggaggcggactcgtgcaggccgagggctccctgcggctgagctgcaccgcatccggcctcacattcgacaactacgccatgggctggttccgccaggccccaggcaaggaaagggagttcgtggcgacgatcagcgcggccggccaccgcaccatcttcaccaacagcgtgcgcggaaggttcaccgccagccgagacaacgccaagaagaccacgtacctgcagatgaactccctgaagcccgaggacaccgccgtgtactactgtgccgccagcccgagccagtacgggagcatgggcaagagcgcctacgactactggggccagggcaccctggtcaccgtgtcgagcggaggcggcggctccggaggcggaggcagcggcggcggcggatcggaggtccagctggtggagagcggcggcgggctcgtgcagcccggcgggagcctcagtctgagctgcgccgccagcggagacaccttcgggaccaaggccatgggctggttcaggcaggcgccgggcaagggccgagaattcgtggccgccatcacgtggaccgccggcctgacctacctggcggactccgccaagggccggtttaccatctccaaggaccgcgtgcagaacaccgtctacctggagatgaacaacctggagcccgaggacacagccgtctactactgcgcctcgcggaggaccttcagctacgaacaccccaactactacaccgcctccgcgtaccacttctggggacagggcacgctggtgaccgtgagcagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 457 - DNA, 1521 bp, synthetic construct
  source 1..1521: mol_type = other DNA
atggagaccgacaccctcctgctgtgggtgctgctgctttgggtgcccgggagcacgggcgaggtccaactggtggagagcggaggcggactcgtgcaggccgagggctccctgcggctgagctgcaccgcatccggcctcacattcgacaactacgccatgggctggttccgccaggccccaggcaaggaaagggagttcgtggcgacgatcagcgcggccggccaccgcaccatcttcaccaacagcgtgcgcggaaggttcaccgccagccgagacaacgccaagaagaccacgtacctgcagatgaactccctgaagcccgaggacaccgccgtgtactactgtgccgccagcccgagccagtacgggagcatgggcaagagcgcctacgactactggggccagggcaccctggtcaccgtgtcgagcggaggcggcggctccggaggcggaggcagcggcggcggcggatcggaggtgcagctcgtcgagagcggcggcggcctggtacagaccggcgacagcctccgtctgacctgcgccgccagcggccgcaccatcggcgaccggtccatggcgtggttcaggcaggccccgggcaaggagcgggagttcgtcatcgtcaacacgtggaacggctacacctacaccgacgagagcgtggagggccgcttcaccatcagcagggacagcggcgccaacacggtgcacctgcagatgaacaagctgaaccccaaggacaccgccgtgtactactgcgcctcctccagctacacctataacatcaggcagcgaagctcgtacgactggtggggccaggggacgctggtgaccgtgagcagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 458 - DNA, 1065 bp, synthetic construct
  source 1..1065: mol_type = other DNA
atggagaccgacaccctcctgctgtgggtgctgctgctttgggtgcccgggagcacgggcgaagtgcagctggtcgagagcggcggcggcctggtgcaggccggcgggagcctcagactgagctgcgccgggtccggcttcaccttctcctcgaccgtgataggctggtaccgccaggcgccgggcaagcagcgggaattcgtggccctgatcaccgccggcggccacaccaacagcgtcgaggaccgattcatcatttccagggacaacgccaagagcaccgcctacctgcagatgaccagcctcaagagcgaggacaccgccgtgtactactgcaacctgcccaaccagggctactggggccaggggaccctggtcacggtgagcagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 459 - protein, 8 aa, synthetic construct
  VARIANT 3: note = X = T or I
  VARIANT 5: note = X = S or R
  VARIANT 6: note = X = S or N
GFXFXXYG

SEQ ID NO: 460 - protein, 8 aa, synthetic construct
  VARIANT 6: note = X = N or S
  VARIANT 7: note = X = K or N
  VARIANT 8: note = X = K or N
IYYDGXXX

SEQ ID NO: 461 - protein, 11 aa, synthetic construct
  VARIANT 11: note = I or L
ARELTGDAFDX

SEQ ID NO: 462 - protein, 6 aa, synthetic construct
  VARIANT 2: note = X = S or R
  VARIANT 6: note = X = S or H
QXIGSX

SEQ ID NO: 463 - protein, 643 aa, synthetic construct
SANDWTVDHPQTLFAWEGACIRIPCKYKTPLPKARLDNILLFQNYEFDKATKKFTGTVLYNATKTEKDPESELYLSKQGRVTFLGNRIDNCTLKIHPIRANDSGNLGLRMTAGTERWMEPIHLNVSEKPFQPYIQMPSEIRESQSVTLTCGLNFSCFGYDILLKWFLEDSEITSITSSVTSITSSVTSSIKNVYTESKLTFQPKWTDHGKSVKCQVQHSSKVLSERTVRLDVKYTPKLEIKVNPTEVEKNNSVTMTCRVNSSNPKLRTVAVSWFKDGRPLEDQELEQEQQMSKLILHSVTKDMRGKYRCQASNDIGPGESEEVELTVHYAPEPSRVHIYPSPAEEGQSVELICESLASPSATNYTWYHNRKPIPGDTQEKLRIPKVSPWHAGNYSCLAENRLGHGKIDQEAKLDVHYAPKAVTTVIQSFTPILEGDSVTLVCRYNSSNPDVTSYRWNPQGSGSVLKPGVLRIQKVTWDSMPVSCAACNHKCSWALPVILNVHYAPRDVKVLKVSPASEIRAGQRVLLQCDFAESNPAEVRFFWKKNGSLVQEGRYLSFGSVSPEDSGNYNCMVNNSIGETLSQAWNLQVLYAPRRLRVSISPGDHVMEGKKATLSCESDANPPISQYTWFDSSGQDLHSSGQK

SEQ ID NO: 464 - protein, 9 aa, synthetic construct
  VARIANT 5: note = X= T, R, or S
HQSSXEPYT

SEQ ID NO: 465 - DNA, 354 bp, synthetic construct
  source 1..354: mol_type = other DNA
caggtgcagctggtggaatctgggggagacgtggtccagcctgggaggtccctgagactctcctgtgcagcgtctggattcatcttcaggaactatggcatgcactgggtccgccaggctccaggcaaggggctggagtgggtggcagtcatttactatgatggaagtaaaaattactatgaagactccgtgaagggccgattcaccatctccagagacaattccaaaaacacgctgtatctgcaaatgaacagcctgagagtcgaggacacggctgtgtattactgtgcgagagaacttactggggatgcttttgatatctggggccaagggacaatggtcaccgtctcttca

SEQ ID NO: 466 - DNA, 321 bp, synthetic construct
  source 1..321: mol_type = other DNA
gaaattgtgctgactcagtctccagactttcagtctctgactccaaaggagaaagtcaccatcacctgtcgggccagtcagagaattggtagtagcttacactggtaccagcagaaaccagatcagtctccaaaactcctcatcaagtatgcttcccagtccttctcaggggtcccctcgaggttcagtggcagtggatctgggacagatttcaccctcaccatcaatagcctggatgctgaagatgctgcaacgtattactgtcatcagagtagtagtttcccgtacacttttggccaggggaccaagctgcagatcaaa

SEQ ID NO: 467 - DNA, 354 bp, synthetic construct
  source 1..354: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcggttatatactatgatggaaataaaaaatactatgttgactccgtgaaggaccgaatcaccatctccagagacaattccaagaacacgttgtatctgcaaatgaacagcctgagagccgaggacacggctgtgtattattgtgcgagagaactaactggggatgcttttgatatctggggccaagggacaatggtcaccgtctcttca

SEQ ID NO: 468 - DNA, 321 bp, synthetic construct
  source 1..321: mol_type = other DNA
gaaattgtgctgactcagtctccagactttcagtctgtgactccaaaggagaaagtcaccatcacctgccgggccagtcagagaattggaagtagcttacactggtaccagcagaaatcagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcatcagagtagtacgttcccgtacacttttggccaggggaccaagctagagatcaaa

SEQ ID NO: 469 - DNA, 354 bp, synthetic construct
  source 1..354: mol_type = other DNA
caggtgcagctggtggagtctggggggggcgtggtccagcctgggaggtccctgagactctcctgtgcagcgtctggattcaccttcagtagctatggcatgcactgggtccgccagactccaggcaaggggctggagtgggtggcagttatatactatgatggaaataataaatactatgcagactccgtgaagggccgattcaccatctccagagagaattccaagaacacgctgtatctgcaaatgaacagcctgagagccgatgacacggctctatattactgtgcgagagaactaactggggatgcttttgatctctggggccaagggacaatggtcaccgtctcttca

SEQ ID NO: 470 - DNA, 321 bp, synthetic construct
  source 1..321: mol_type = other DNA
gaaattgtgctgactcagtctccagattttcagtctgcgactccaaaggagaaagtcaccatcacctgccgggccagtcagagcattggtagtcacttacactggtatcagcagaaaccagatcagtctccaaagctcctcatcaagtatgcttcccagtccttctcaggggtcccctcgaggttcagtggcagtggatctgggacagatttcaccctcaccatcaatagactggaagatgaagatgctgcaacgtattactgtcatcagagtagtaggttcccgtacacttttggccaggggaccaagctggatatcaaa

SEQ ID NO: 471 - protein, 668 aa, synthetic construct
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 472 - protein, 699 aa, synthetic construct
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRGGGGSGLNDIFEAQKIEWHEGGGGSHHHHHH

SEQ ID NO: 473 - protein, 391 aa, synthetic construct
QYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRGGGGSGLNDIFEAQKIEWHEGGGGSHHHHHH

SEQ ID NO: 474 - protein, 342 aa, synthetic construct
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAGGGGSGLNDIFEAQKIEWHEGGGGSHHHHHH

SEQ ID NO: 475 - protein, 5 aa, synthetic construct
TKAMG

SEQ ID NO: 476 - protein, 17 aa, synthetic construct
AITWTAGLTYYADSVKG

SEQ ID NO: 477 - protein, 19 aa, synthetic construct
RRTFSYEHPNYYTRSAYHF

SEQ ID NO: 478-480 - skipped (3 entries)

SEQ ID NO: 481 - protein, 128 aa, synthetic construct
EVQLVESGGGVVQPGGSLRLSCAASGDTFGTKAMGWFRQAPGKGREFVAAITWTAGLTYYADSVKGRFTISKDRAKNTVDLQMNSLRPEDTALYYCASRRTFSYEHPNYYTRSAYHFWGQGTLVTVSS

SEQ ID NO: 482 - DNA, 1089 bp, synthetic construct
  source 1..1089: mol_type = other DNA
atggagaccgacaccctcctgctgtgggtgctgctgctttgggtgcccgggagcacgggcgaggtccagctggtcgagagtggcggaggcctggtgcagcccggcggcagcctgagactgttctgcgccgccagcggcagcaggctgagcttcaccaccatgggctggtaccgccaggccccaggcaagcagcgagaggtggtggcctatatcacggagagcggctcgaccgcctacaccgccagtgtgaaggacaggttcaccatctcccgggacaacgccaagaataccgtgtccctccagatgaacagcctgaagccggaggacaccgccgtgtacttctgcaacgcggtgatcatgatgggcgggagcggcacctactggggccccgggacgctcgtgaccgtcagctccgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 483 - DNA, 1107 bp, synthetic construct
  source 1..1107: mol_type = other DNA
atggagaccgacaccctcctgctgtgggtgctgctgctttgggtgcccgggagcacgggcgaggtgcagctggtggagtccggcggcgggctggtgcaggccggcgacagcctgaggctctcgtgcgccgccagcggcaggaccctgagcagctacacgaccggctggttccgtcaggggctgggcaaggagcgcgagttcgtggccgcgatcaccaagaacggcccctacctctactacaaggacagcgtcaggggccgcttcaccatctcccgagacagcgccaagaccaccgtgttcctgcagatggacaacctgaagccggaggacaccgccgtctattactgcgccgtaagcgacgcccagtacgacatccggaaccgggagaagtactactactggggccagggcaccctggtgacggtctccagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 484 - DNA, 1188 bp, synthetic construct
  source 1..1188: mol_type = other DNA
atggagaccgacaccctcctgctgtgggtgctgctgctttgggtgcccgggagcacgggcgaggtccagctggtggagagcggcggcggcctggtgcaggccggcgacagcctgcgcctgtcctgcgccgccagcggcaggaccctgagcagctacacgaccggctggttccgacagggcctcgggaaggagagggaattcgtcgccgccatcaccaagaacggcccgtacctctactacaaggactccgtccgcggccgcttcaccatcagccgggactccgccaagaccaccgtgttcctgcagatggacaacctgaagcccgaggacaccgcggtgtactactgcgccgtgtcggacgcccagtacgacatccgtaacagagagaagtactactactgggggcagggcaccctggtgacggtgagcagcgccagccttagcaacagcatcatgtacttcagccactttgtcccggtcttcctgccagccaagccgaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 485 - DNA, 1524 bp, synthetic construct
  source 1..1524: mol_type = other DNA
atggagaccgacaccctcctgctgtgggtgctgctgctttgggtgcccgggagcacgggcgaggtgcagctggtggagtccggcggcgggctggtgcaggccggcgacagcctgaggctctcgtgcgccgccagcggcaggaccctgagcagctacacgaccggctggttccgtcaggggctgggcaaggagcgcgagttcgtggccgcgatcaccaagaacggcccctacctctactacaaggacagcgtcaggggccgcttcaccatctcccgagacagcgccaagaccaccgtgttcctgcagatggacaacctgaagccggaggacaccgccgtctattactgcgccgtaagcgacgcccagtacgacatccggaaccgggagaagtactactactggggccagggcaccctggtgacggtctccagcggaggcggcggctccggaggcggaggcagcggcggcggcggatcggaggtccaactggtggagagcggaggcggactcgtgcaggccgagggctccctgcggctgagctgcaccgcatccggcctcacattcgacaactacgccatgggctggttccgccaggccccaggcaaggaaagggagttcgtggcgacgatcagcgcggccggccaccgcaccatcttcaccaacagcgtgcgcggaaggttcaccgccagccgagacaacgccaagaagaccacgtacctgcagatgaactccctgaagcccgaggacaccgccgtgtactactgtgccgccagcccgagccagtacgggagcatgggcaagagcgcctacgactactggggccagggcaccctggtcaccgtgtcgagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 486 - DNA, 1521 bp, synthetic construct
  source 1..1521: mol_type = other DNA
atggagaccgacaccctcctgctgtgggtgctgctgctttgggtgcccgggagcacgggcgaggtgcagctggtggagtccggcggcgggctggtgcaggccggcgacagcctgaggctctcgtgcgccgccagcggcaggaccctgagcagctacacgaccggctggttccgtcaggggctgggcaaggagcgcgagttcgtggccgcgatcaccaagaacggcccctacctctactacaaggacagcgtcaggggccgcttcaccatctcccgagacagcgccaagaccaccgtgttcctgcagatggacaacctgaagccggaggacaccgccgtctattactgcgccgtaagcgacgcccagtacgacatccggaaccgggagaagtactactactggggccagggcaccctggtgacggtctccagcggaggcggcggctccggaggcggaggcagcggcggcggcggatcggaggtgcagctcgtcgagagcggcggcggcctggtacagaccggcgacagcctccgtctgacctgcgccgccagcggccgcaccatcggcgaccggtccatggcgtggttcaggcaggccccgggcaaggagcgggagttcgtcatcgtcaacacgtggaacggctacacctacaccgacgagagcgtggagggccgcttcaccatcagcagggacagcggcgccaacacggtgcacctgcagatgaacaagctgaaccccaaggacaccgccgtgtactactgcgcctcctccagctacacctataacatcaggcagcgaagctcgtacgactggtggggccaggggacgctggtgaccgtgagcagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 487 - DNA, 1188 bp, synthetic construct
  source 1..1188: mol_type = other DNA
atggagaccgacaccctcctgctgtgggtgctgctgctttgggtgcccgggagcacgggcgaggtgcagctggtggagtccggcggcggcctggtgccgccgggcggctccctcaggctcagttgcgcggccagcgggcggacgggagacatcaacggcatgggctggttcaggcgcgccgccggcaaagaaagagagttcgtggccgccacgcggtggaacggcggcaccaagtactacaccagcagcgtcgagggcagattcaccataagcggcgacagggtcggaaacaccgtgaccctggaaatgaataacctggtgcccgaggacacggcccgctattactgtgcaaccacccgacgtcggttcgacttctatagcgtcggcgggtacgagaactggggccagggcacccaggtgaccgtgagcagcgccagccttagcaacagcatcatgtacttcagccactttgtcccggtcttcctgccagccaagccgaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 488 - DNA, 1128 bp, synthetic construct
  source 1..1128: mol_type = other DNA
atggagaccgacaccctcctgctgtgggtgctgctgctttgggtgcccgggagcacgggcgaggtgcagctggtggagagcggcggcgggctcgtgccgcccggcgggagcctacggctgagctgcgccgcatccggccggaccggagacatcaacggcatgggctggtttcgccgagccgcagggaaggaacgggagttcgtcgccgccacgcgttggaacggcggaaccaaatactatacaagcagtgtcgagggcaggttcaccatctccggggaccgagtgggcaacaccgtgaccctggagatgaacaacctcgtgccggaggatacggccaggtactactgcgcgaccacccgccgcaggttcgatttctacagcgtgggcggatacgagaactggggccagggcacccaggtgaccgtcagcagcgcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 489 - DNA, 1119 bp, synthetic construct
  source 1..1119: mol_type = other DNA
atggagaccgacaccctcctgctgtgggtgctgctgctttgggtgcccgggagcacgggcgaggtccagctggtggagagcggcggcgggctcgtgcagcccggcgggagcctcagtctgagctgcgccgccagcggagacaccttcgggaccaaggccatgggctggttcaggcaggcgccgggcaagggccgagaattcgtggccgccatcacgtggaccgccggcctgacctacctggcggactccgccaagggccggtttaccatctccaaggaccgcgtgcagaacaccgtctacctggagatgaacaacctggagcccgaggacacagccgtctactactgcgcctcgcggaggaccttcagctacgaacaccccaactactacaccgcctccgcgtaccacttctggggacagggcacgctggtgaccgtgagcagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 490 - DNA, 1536 bp, synthetic construct
  source 1..1536: mol_type = other DNA
atggagaccgacaccctcctgctgtgggtgctgctgctttgggtgcccgggagcacgggcgaggtccagctggtggagagcggcggcgggctcgtgcagcccggcgggagcctcagtctgagctgcgccgccagcggagacaccttcgggaccaaggccatgggctggttcaggcaggcgccgggcaagggccgagaattcgtggccgccatcacgtggaccgccggcctgacctacctggcggactccgccaagggccggtttaccatctccaaggaccgcgtgcagaacaccgtctacctggagatgaacaacctggagcccgaggacacagccgtctactactgcgcctcgcggaggaccttcagctacgaacaccccaactactacaccgcctccgcgtaccacttctggggacagggcacgctggtgaccgtgagcagcggaggcggcggctccggaggcggaggcagcggcggcggcggatcggaggtccaactggtggagagcggaggcggactcgtgcaggccgagggctccctgcggctgagctgcaccgcatccggcctcacattcgacaactacgccatgggctggttccgccaggccccaggcaaggaaagggagttcgtggcgacgatcagcgcggccggccaccgcaccatcttcaccaacagcgtgcgcggaaggttcaccgccagccgagacaacgccaagaagaccacgtacctgcagatgaactccctgaagcccgaggacaccgccgtgtactactgtgccgccagcccgagccagtacgggagcatgggcaagagcgcctacgactactggggccagggcaccctggtcaccgtgtcgagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 491 - DNA, 1107 bp, synthetic construct
  source 1..1107: mol_type = other DNA
atggagaccgacaccctcctgctgtgggtgctgctgctttgggtgcccgggagcacgggcgaggtgcaactcgtggagtccggcggcggcctggtgcagccgggcgggagcctgcggctgtcgtgcgtggccagcggcctgttcctgagcgactacacgctggcctggttccggcaggcgcccggcaaggagagggagttcgtgatcgtgagcacctggaacggctacacctactacggcgagagcgtctccggcaggttcaccatcagccgcgacaacgccgagaagaccgtgttcctgcagatgaacagcctgaagcccgaggacaccgccgtctactactgcgccgccaagttccgaatcagctacaacgtgcgcgacgacagcttctacgacctctggggccagggcaccctcgtgaccgtctccagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 492 - DNA, 1128 bp, synthetic construct
  source 1..1128: mol_type = other DNA
atggagaccgacaccctcctgctgtgggtgctgctgctttgggtgcccgggagcacgggcgaggtccagctggtcgagtcgggcggcggcctggtgcagccgggcggctccctgcggctgtcctgcgtggccagcgggctgttcctcagcgactacaccctggcctggttcaggcaggcccccgggaaggagcgcgagttcgtgatcgtgagcacctggaacggctacacctactacggcgagtccgtgagcggcaggttcaccatcagccgcgacaacgccgagaagaccgtgttcctgcagatgaacagcctgaagcccgaggacaccgccgtgtactactgcgcggcgaagttccgaatcagctacaacgtgcgggacgacagcttctacgacctctggggccagggcaccctcgtgacggtcagcagcgcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacaagcgcggcaggaagaagctcctgtacatcttcaagcagccgttcatgcgcccggtgcagacgacccaggaggaggacgggtgcagctgccggttcccggaggaggaggagggcggctgcgagctgagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 493 - DNA, 1128 bp, synthetic construct
  source 1..1128: mol_type = other DNA
atggagaccgacaccctcctgctgtgggtgctgctgctttgggtgcccgggagcacgggcgaggtccagctggtcgagtcgggcggcggcctggtgcagccgggcggctccctgcggctgtcctgcgtggccagcgggctgttcctcagcgactacaccctggcctggttcaggcaggcccccgggaaggagcgcgagttcgtgatcgtgagcacctggaacggctacacctactacggcgagtccgtgagcggcaggttcaccatcagccgcgacaacgccgagaagaccgtgttcctgcagatgaacagcctgaagcccgaggacaccgccgtgtactactgcgcggcgaagttccgaatcagctacaacgtgcgggacgacagcttctacgacctctggggccagggcaccctcgtgacggtcagcagcgcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 494 - DNA, 1128 bp, synthetic construct
  source 1..1128: mol_type = other DNA
atggagaccgacaccctcctgctgtgggtgctgctgctttgggtgcccgggagcacgggcgaggtgcagctggtggagagcggaggcggactggtgcaggcgggcggcagcctgacgctcacctgtgccgcctccggcctcaccttcgcaggatatagcgtcgggtggttccggcaggccccaggcaaggagagggagttcgtggccgtgatcacccgtggcggagactaccgctacttcgccgagagcgtgcagggccggttcaccgtgagccgagataacgggaagaacaccgtcagcctgcagatgctggacctgaagcccgacgacaccgccgtctactactgcgccgcaggcaccatgacctacgcaatccgcgagacgagggcgtacaactactggggccaagggaccctggtgaccgtgagttccgcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 495 - DNA, 1104 bp, synthetic construct
  source 1..1104: mol_type = other DNA
atggagaccgacaccctcctgctgtgggtgctgctgctttgggtgcccgggagcacgggcgaggtgcagctcgtcgagagcggcggcggcctggtacagaccggcgacagcctccgtctgacctgcgccgccagcggccgcaccatcggcgaccggtccatggcgtggttcaggcaggccccgggcaaggagcgggagttcgtcatcgtcaacacgtggaacggctacacctacaccgacgagagcgtggagggccgcttcaccatcagcagggacagcggcgccaacacggtgcacctgcagatgaacaagctgaaccccaaggacaccgccgtgtactactgcgcctcctccagctacacctataacatcaggcagcgaagctcgtacgactggtggggccaggggacgctggtgaccgtgagcagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 496 - DNA, 1521 bp, synthetic construct
  source 1..1521: mol_type = other DNA
atggagaccgacaccctcctgctgtgggtgctgctgctttgggtgcccgggagcacgggcgaggtgcagctcgtcgagagcggcggcggcctggtacagaccggcgacagcctccgtctgacctgcgccgccagcggccgcaccatcggcgaccggtccatggcgtggttcaggcaggccccgggcaaggagcgggagttcgtcatcgtcaacacgtggaacggctacacctacaccgacgagagcgtggagggccgcttcaccatcagcagggacagcggcgccaacacggtgcacctgcagatgaacaagctgaaccccaaggacaccgccgtgtactactgcgcctcctccagctacacctataacatcaggcagcgaagctcgtacgactggtggggccaggggacgctggtgaccgtgagcagcggaggcggcggctccggaggcggaggcagcggcggcggcggatcggaggtccaactggtggagagcggaggcggactcgtgcaggccgagggctccctgcggctgagctgcaccgcatccggcctcacattcgacaactacgccatgggctggttccgccaggccccaggcaaggaaagggagttcgtggcgacgatcagcgcggccggccaccgcaccatcttcaccaacagcgtgcgcggaaggttcaccgccagccgagacaacgccaagaagaccacgtacctgcagatgaactccctgaagcccgaggacaccgccgtgtactactgtgccgccagcccgagccagtacgggagcatgggcaagagcgcctacgactactggggccagggcaccctggtcaccgtgtcgagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 497 - DNA, 1521 bp, synthetic construct
  source 1..1521: mol_type = other DNA
atggagaccgacaccctcctgctgtgggtgctgctgctttgggtgcccgggagcacgggcgaggtgcagctcgtcgagagcggcggcggcctggtacagaccggcgacagcctccgtctgacctgcgccgccagcggccgcaccatcggcgaccggtccatggcgtggttcaggcaggccccgggcaaggagcgggagttcgtcatcgtcaacacgtggaacggctacacctacaccgacgagagcgtggagggccgcttcaccatcagcagggacagcggcgccaacacggtgcacctgcagatgaacaagctgaaccccaaggacaccgccgtgtactactgcgcctcctccagctacacctataacatcaggcagcgaagctcgtacgactggtggggccaggggacgctggtgaccgtgagcagcggaggcggcggctccggaggcggaggcagcggcggcggcggatcggaggtgcagctggtggagtccggcggcgggctggtgcaggccggcgacagcctgaggctctcgtgcgccgccagcggcaggaccctgagcagctacacgaccggctggttccgtcaggggctgggcaaggagcgcgagttcgtggccgcgatcaccaagaacggcccctacctctactacaaggacagcgtcaggggccgcttcaccatctcccgagacagcgccaagaccaccgtgttcctgcagatggacaacctgaagccggaggacaccgccgtctattactgcgccgtaagcgacgcccagtacgacatccggaaccgggagaagtactactactggggccagggcaccctggtgacggtctccagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 498 - DNA, 1104 bp, synthetic construct
  source 1..1104: mol_type = other DNA
atggagaccgacaccctcctgctgtgggtgctgctgctttgggtgcccgggagcacgggcgaggtccagctggtcgagtccggcggcggccaggtgcaggccgggggcagcctgcgcctcagctgcgtgcactcgggacgaacgttctcctcctacgtggcagcgtggtttcggcagtaccccggcaaggagagggagttcgtggccgcggccagcctcagcggcggcaccaccgattacgccgacagcgtgaagggccggttcaccatcacaagggacaacgccgcaagcacgcactacctgcagatgaacagcctgcagccggaggacaccgccgtctactactgcgccgccagacccgtgtacctgaagtacacctaccacgattacgacttttggggccaaggcaccctggtgaccgtgagcagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 499 - DNA, 1125 bp, synthetic construct
  source 1..1125: mol_type = other DNA
atggagaccgacaccctcctgctgtgggtgctgctgctttgggtgcccgggagcacgggcgaggtgcagctggtggagagcggcggcggcctggtccaggccggcgacagcctgaggctcagctgcagcctgagcggccgcaccttctccagctacgccatggcctggttccggcagctgcccgggaaggagcgagagttcgtgagcgtgatcagcctgtccggccagaacacgtactacgccgacagcgtcaagggccgcttcaccatcagccgggacaactcgaagtccaccgtgttcctccagatgaacagcctgaagcccgaggacaccgccgtgtactactgcgccgcgaggccggtctaccacaagttccactaccgcgactacgacttctggggccagggcaccctggtgaccgtgagctccgcgagcaccaccacgcccgcgccgcggccgccgacgccggcgcccacaatcgcctcacagccactgagcctgcgcccggaggcctgcaggcccgccgcaggcggcgccgtgcacacgcgtgggctcgacttcgcctgtgacatatacatctgggcgcccctggccggcacctgcggcgtcctgctgctgagcctggtgatcaccctgtattgcaaccaccggaacgtgcgctccaaacgcagccggctgctgcacagcgactacatgaacatgacgccgcgcaggcccggcccgacccgaaagcactaccagccatacgcgccgccgcgggacttcgccgcctaccggtcaagggttaagttctcacggtcggccgacgcgcccgcctaccagcagggccagaaccagctgtacaatgagctgaacctgggccggagggaggagtacgacgtactggataagaggcgcggcagagaccccgagatgggcggcaaacccaggcgtaagaacccgcaggagggcctgtacaacgaactgcagaaggacaagatggccgaggcctacagcgaaataggcatgaagggcgagcgtaggcgcgggaagggccacgacggcctgtatcaaggcctgtcgaccgccaccaaagacacctacgacgccctgcacatgcaggccctgccgcccaggtagtgatga

SEQ ID NO: 500 - DNA, 1071 bp, synthetic construct
  source 1..1071: mol_type = other DNA
atggagaccgacaccctcctgctgtgggtgctgctgctttgggtgcccgggagcacgggcgaggtccagctcgtggagagcggaggcgggctcgtgcagcccggcgggagcatgcggctgtcgtgccagggctccgggcgcagcttcaggctgaataccatggtgtggtaccgtcgagcgagcggcaaccagagggagctggtcgccagcctgacctccggcggcaacacctactacgccgactccgtgaagggccgcttcacaatcagccgggacgacgccaagagcaccgtcgacctacagatgaacagcctgaggccggccgacaccgcagtgtacttctgctacatgggcggaaacagctggggccaggaaacgcaggtgaccgtgagcagcgcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccgcgccgcccgggcccgacccggaagcactaccagccctacgcgccgccgcgcgacttcgcggcctaccgctcccgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 501 - DNA, 1110 bp, synthetic construct
  source 1..1110: mol_type = other DNA
atggccctgcccgtgaccgccctgctgctgccactggccctgctgctgcacgccgcccggcccgaggtgcagctggtggagtctggcggcggagtggtgcagcccggcggcagcctgagactgagctgtgccgcctccggcctatttctaagcgactacacgctagcctggtttcgccaggcccccggcaaggagcgcgagttcgtgatcgtttccacgtggaaccgctatacctactatgccgaaggagtgaagggcaggttcaccatcagcagagacaacgcaaaaaaaaccgtgtatctgcagatgaatagcctgaggcctgaggataccgccctgtactactgcgccgccaaattccgcatctcatacaacgttagggacgacagcttttacgacctgtggggccagggcaccctggtgaccgtgagctccgccgccgccatcgaggtgatgtacccccccccttacctggacaacgagaagtccaacggcaccatcatccacgtgaaaggcaagcacctgtgtcccagccctctgttccccggcccaagcaagcctttctgggtgctggtggtggtgggcggcgtgctggcctgctactccctgctggtgaccgtggccttcatcatcttctgggttagaagcaagcgctcacgcctgctacactcagactacatgaacatgacgcccagaagacccggacctaccagaaagcactaccaaccatatgccccaccacgcgactttgcagcatacagaagccgggtgaaattcagccggagcgccgacgcccccgcctaccagcagggccagaaccagctgtataacgagctgaacctgggcagaagggaggagtacgacgtgctggacaagcgcaggggccgcgaccctgagatgggcggcaagcccaggagaaagaacccacaggagggcctgtataacgagctgcagaaggacaagatggccgaggcctacagcgagatcggcatgaagggagagcgcaggagaggcaagggccacgacggcctgtaccagggcctgtccaccgccacaaaggacacatacgacgccctgcacatgcaggccctgccccccaggtaataataa

SEQ ID NO: 502 - DNA, 1110 bp, synthetic construct
  source 1..1110: mol_type = other DNA
atggccctgcccgtgaccgccctgctgctgccactggccctgctgctgcacgccgcccggcccgaggtgcagctggtggagtctggcggcggagtggtgcagcccggcggcagcctgagactgagctgtgccgcctccggcctatttctaagcgactacacgctagcctggtttcgccaggcccccggcaaggagcgcgagttcgtgatcgtttccacgtggaaccgctatacctactatgccgaaggagtgaagggcaggttcaccatcagcagagacaacgcaaaaaaaaccgtgtatctgcagatgaatagcctgaggcctgaggataccgccctgtactactgcgccgccaaattccgcatctcatacaacgttagggacgacagcttttacgacctgtggggccagggcaccctggtgaccgtgagctccgccgccgccatcgaggtgatgtacccccccccttacctggacaacgagaagtccaacggcaccatcatccacgtgaaaggcaagcacctgtgtcccagccctctgttccccggcccaagcaagcctttctgggtgctggtggtggtgggcggcgtgctggcctgctactccctgctggtgaccgtggccttcatcatcttctggaagagaggacgcaagaagctgctgtacatcttcaagcaaccattcatgcggcccgttcaaacgacccaagaagaagacggctgtagctgtaggtttccagaggaagaagagggcggctgtgaactacgggtgaaattcagccggagcgccgacgcccccgcctaccagcagggccagaaccagctgtataacgagctgaacctgggcagaagggaggagtacgacgtgctggacaagcgcaggggccgcgaccctgagatgggcggcaagcccaggagaaagaacccacaggagggcctgtataacgagctgcagaaggacaagatggccgaggcctacagcgagatcggcatgaagggagagcgcaggagaggcaagggccacgacggcctgtaccagggcctgtccaccgccacaaaggacacatacgacgccctgcacatgcaggccctgccccccaggtaataataa

SEQ ID NO: 503 - DNA, 1122 bp, synthetic construct
  source 1..1122: mol_type = other DNA
atggccctgcccgtgaccgccctgctgctgccactggccctgctgctgcacgccgcccggcccgaggtgcagctggtggagtctggcggcggagtggtgcagcccggcggcagcctgagactgagctgtgccgcctccggcgacaccttcggaacgaaagccatgggatggtttcgccaggcccccggcaagggacgcgagttcgtggccgcaatcacgtggaccgcaggcctaacctactatgccgacagcgtgaagggcaggttcaccatcagcaaagaccgcgcaaaaaacaccgtggacctgcagatgaatagcctgaggcctgaggataccgccctgtactactgcgcctcacggcgcacgttttcatacgagcaccctaactactataccaggtcagcctaccacttttggggccagggcaccctggtgaccgtgagctccgccgccgccatcgaggtgatgtacccccccccttacctggacaacgagaagtccaacggcaccatcatccacgtgaaaggcaagcacctgtgtcccagccctctgttccccggcccaagcaagcctttctgggtgctggtggtggtgggcggcgtgctggcctgctactccctgctggtgaccgtggccttcatcatcttctgggttagaagcaagcgctcacgcctgctacactcagactacatgaacatgacgcccagaagacccggacctaccagaaagcactaccaaccatatgccccaccacgcgactttgcagcatacagaagccgggtgaaattcagccggagcgccgacgcccccgcctaccagcagggccagaaccagctgtataacgagctgaacctgggcagaagggaggagtacgacgtgctggacaagcgcaggggccgcgaccctgagatgggcggcaagcccaggagaaagaacccacaggagggcctgtataacgagctgcagaaggacaagatggccgaggcctacagcgagatcggcatgaagggagagcgcaggagaggcaagggccacgacggcctgtaccagggcctgtccaccgccacaaaggacacatacgacgccctgcacatgcaggccctgccccccaggtaataataa

SEQ ID NO: 504 - DNA, 1122 bp, synthetic construct
  source 1..1122: mol_type = other DNA
atggccctgcccgtgaccgccctgctgctgccactggccctgctgctgcacgccgcccggcccgaggtgcagctggtggagtctggcggcggagtggtgcagcccggcggcagcctgagactgagctgtgccgcctccggcgacaccttcggaacgaaagccatgggatggtttcgccaggcccccggcaagggacgcgagttcgtggccgcaatcacgtggaccgcaggcctaacctactatgccgacagcgtgaagggcaggttcaccatcagcaaagaccgcgcaaaaaacaccgtggacctgcagatgaatagcctgaggcctgaggataccgccctgtactactgcgcctcacggcgcacgttttcatacgagcaccctaactactataccaggtcagcctaccacttttggggccagggcaccctggtgaccgtgagctccgccgccgccatcgaggtgatgtacccccccccttacctggacaacgagaagtccaacggcaccatcatccacgtgaaaggcaagcacctgtgtcccagccctctgttccccggcccaagcaagcctttctgggtgctggtggtggtgggcggcgtgctggcctgctactccctgctggtgaccgtggccttcatcatcttctggaagagaggacgcaagaagctgctgtacatcttcaagcaaccattcatgcggcccgttcaaacgacccaagaagaagacggctgtagctgtaggtttccagaggaagaagagggcggctgtgaactacgggtgaaattcagccggagcgccgacgcccccgcctaccagcagggccagaaccagctgtataacgagctgaacctgggcagaagggaggagtacgacgtgctggacaagcgcaggggccgcgaccctgagatgggcggcaagcccaggagaaagaacccacaggagggcctgtataacgagctgcagaaggacaagatggccgaggcctacagcgagatcggcatgaagggagagcgcaggagaggcaagggccacgacggcctgtaccagggcctgtccaccgccacaaaggacacatacgacgccctgcacatgcaggccctgccccccaggtaataataa

SEQ ID NO: 505 - DNA, 1107 bp, synthetic construct
  source 1..1107: mol_type = other DNA
atggccctgcccgtgaccgccctgctgctgccactggccctgctgctgcacgccgcccggcccgaggtgcagctggtggagtctggcggcggagtggtgcagcccggcggcagcctgagactgagctgtgccgcctccggctttaccttcagcagctacgccatggcctggtttcgccaggcccccggcaaggagcgcgagttcgtggccgtgatctccctatcaggcggcagcgttgactatcgcgacagcgtgaagggcaggttcaccatcagcagagacaacgcaaaaaacaccgtggacctgcagatgaatagcctgaggcctgaggataccgccctgtactactgcgccgcccggcctgtttatatcaaatggtccgaaagggactttgactattggggccagggcaccctggtgaccgtgagctccgccgccgccatcgaggtgatgtacccccccccttacctggacaacgagaagtccaacggcaccatcatccacgtgaaaggcaagcacctgtgtcccagccctctgttccccggcccaagcaagcctttctgggtgctggtggtggtgggcggcgtgctggcctgctactccctgctggtgaccgtggccttcatcatcttctgggttagaagcaagcgctcacgcctgctacactcagactacatgaacatgacgcccagaagacccggacctaccagaaagcactaccaaccatatgccccaccacgcgactttgcagcatacagaagccgggtgaaattcagccggagcgccgacgcccccgcctaccagcagggccagaaccagctgtataacgagctgaacctgggcagaagggaggagtacgacgtgctggacaagcgcaggggccgcgaccctgagatgggcggcaagcccaggagaaagaacccacaggagggcctgtataacgagctgcagaaggacaagatggccgaggcctacagcgagatcggcatgaagggagagcgcaggagaggcaagggccacgacggcctgtaccagggcctgtccaccgccacaaaggacacatacgacgccctgcacatgcaggccctgccccccaggtaataataa

SEQ ID NO: 506 - DNA, 1107 bp, synthetic construct
  source 1..1107: mol_type = other DNA
atggccctgcccgtgaccgccctgctgctgccactggccctgctgctgcacgccgcccggcccgaggtgcagctggtggagtctggcggcggagtggtgcagcccggcggcagcctgagactgagctgtgccgcctccggctttaccttcagcagctacgccatggcctggtttcgccaggcccccggcaaggagcgcgagttcgtggccgtgatctccctatcaggcggcagcgttgactatcgcgacagcgtgaagggcaggttcaccatcagcagagacaacgcaaaaaacaccgtggacctgcagatgaatagcctgaggcctgaggataccgccctgtactactgcgccgcccggcctgtttatatcaaatggtccgaaagggactttgactattggggccagggcaccctggtgaccgtgagctccgccgccgccatcgaggtgatgtacccccccccttacctggacaacgagaagtccaacggcaccatcatccacgtgaaaggcaagcacctgtgtcccagccctctgttccccggcccaagcaagcctttctgggtgctggtggtggtgggcggcgtgctggcctgctactccctgctggtgaccgtggccttcatcatcttctggaagagaggacgcaagaagctgctgtacatcttcaagcaaccattcatgcggcccgttcaaacgacccaagaagaagacggctgtagctgtaggtttccagaggaagaagagggcggctgtgaactacgggtgaaattcagccggagcgccgacgcccccgcctaccagcagggccagaaccagctgtataacgagctgaacctgggcagaagggaggagtacgacgtgctggacaagcgcaggggccgcgaccctgagatgggcggcaagcccaggagaaagaacccacaggagggcctgtataacgagctgcagaaggacaagatggccgaggcctacagcgagatcggcatgaagggagagcgcaggagaggcaagggccacgacggcctgtaccagggcctgtccaccgccacaaaggacacatacgacgccctgcacatgcaggccctgccccccaggtaataataa

SEQ ID NO: 507 - DNA, 1122 bp, synthetic construct
  source 1..1122: mol_type = other DNA
atggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcgacaccttcggaacgaaagccatgggatggttcaggcaggcccccggcaagggacgcgagtttgtggccgcaatcacgtggacagcaggcctaacctactacgctgactccgtgaagggcaggttcacaatttctaaagaccgcgccaagaacaccgtggatctgcagatgaactccctgcgccccgaggacacagccctgtactactgcgcctcacggcgcacgttctcatacgagcaccccaattactataccaggtcagcctaccacttttggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctgggttcggtccaagcgctcacgcctgctacactcagactacatgaacatgacgccccgcagacctggacctaccaggaaacactatcaaccatatgcccccccaagggactttgcagcatacaggagccgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataa

SEQ ID NO: 508 - DNA, 1107 bp, synthetic construct
  source 1..1107: mol_type = other DNA
atggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggctttaccttcagctcctacgccatggcctggttcaggcaggcccccggcaaggagcgcgagtttgtggccgtgatcagcctatcaggcggcagcgttgactaccgcgactccgtgaagggcaggttcacaatttctcgggacaacgccaagaacaccgtggatctgcagatgaactccctgcgccccgaggacacagccctgtactactgcgccgcacggcccgtttatatcaaatggagcgaaagggactttgactattggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctgggttcggtccaagcgctcacgcctgctacactcagactacatgaacatgacgccccgcagacctggacctaccaggaaacactatcaaccatatgcccccccaagggactttgcagcatacaggagccgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataa

SEQ ID NO: 509 - DNA, 1107 bp, synthetic construct
  source 1..1107: mol_type = other DNA
atggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggctttaccttcagctcctacgccatggcctggttcaggcaggcccccggcaaggagcgcgagtttgtggccgtgatcagcctatcaggcggcagcgttgactaccgcgactccgtgaagggcaggttcacaatttctcgggacaacgccaagaacaccgtggatctgcagatgaactccctgcgccccgaggacacagccctgtactactgcgccgcacggcccgtttatatcaaatggagcgaaagggactttgactattggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctggaagcggggacgcaagaagctgctgtatatctttaagcaaccattcatgcggcccgttcaaacgactcaagaagaagacgggtgtagctgccggtttcccgaagaagaagagggcggctgtgaactacgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataa

SEQ ID NO: 510 - DNA, 1476 bp, synthetic construct
  source 1..1476: mol_type = other DNA
atgttgctgctcgtgacatctctgctcctctgtgagctcccccatccagcatttctgctcatccccgacatccagatgacacagacaaccagcagcctgagcgccagcctgggcgacagagtgaccatctcctgccgcgccagccaggatatctccaagtacctgaactggtaccagcagaagcccgacggcacagtgaagctgctgatctaccacaccagcaggctgcactccggcgtgccctccaggttcagcggctctggcagcgggaccgattacagcctgaccatcagcaatctggagcaggaggacatcgccacctacttctgccagcagggcaacaccctgccctacacattcggcggcggcaccaagctggagatcaccggctccacctccggctctggcaagcccggcagcggcgagggctccaccaagggcgaggtgaagctgcaggagagcggccccggcctggtggcccccagccagagtctgtccgtgacctgcacagtgagcggcgtgtccctgccagactatggcgtgagctggatcaggcagccacccaggaagggcctggagtggctgggcgtgatctggggaagcgagactacctattacaactccgccctgaagtccaggctgaccatcatcaaggacaacagcaagtcccaggtgttcctgaagatgaactccctgcagaccgatgacaccgccatctactactgcgccaaacactactactacggcggcagctacgccatggactactggggccagggcaccagcgtgaccgtgagcagcgccgccgccatcgaggtgatgtacccacccccatacctggataacgagaagtccaatggcaccatcatccacgtgaagggcaagcacctgtgtccctcccccctgttccccggccccagcaagcccttctgggtgctggtggtggtgggcggcgtgctggcctgctacagcctgctggtgacagtggccttcatcatcttctgggtgcgcagcaagaggtctaggctgctgcacagcgactacatgaatatgaccccccggcggcccggccccaccagaaagcactatcagccctacgcccccccaagagacttcgccgcctacagaagccgggtgaagttcagtagaagcgccgacgcccccgcctaccagcagggccagaaccagctgtacaatgagctgaatctgggccgccgcgaggagtacgacgtgctggataagagaagaggcagggaccccgagatgggcggcaagcctaggagaaagaaccctcaggaggggctgtacaacgagctgcagaaggataagatggccgaggcctacagcgagatcggcatgaagggggagagaaggagaggcaagggccacgacggcctgtaccagggcctgagcaccgctaccaaggacacctacgacgccctgcacatgcaggccctgccccccagataataataa

SEQ ID NO: 511 - DNA, 1476 bp, synthetic construct
  source 1..1476: mol_type = other DNA
atgttgctgttggtcaccagcttgctgttatgtgaattgcctcacccggccttcctcttgatccccgacatccagatgacccagaccacctcttccctgtccgcctccctgggcgatagggtgaccatcagctgcagggcctcccaggacatcagcaagtacctgaattggtaccagcagaagcccgatggaaccgtgaagctgctgatctaccacacctcccgcctgcacagcggcgtgcccagcaggttctccggcagcggctccggaaccgattacagcctgaccatcagcaacctggagcaggaggacatcgccacctacttttgccagcaggggaacaccctgccctacacattcggcggcggcaccaagctggagatcaccggctccacctccggctctggcaagcccggcagcggcgagggctccaccaagggcgaggtgaagctgcaggagagcggccccggcctggtggcccccagccagagtctgtccgtgacctgcacagtgagcggcgtgtccctgccagactatggcgtgagctggatcaggcagccacccaggaagggcctggagtggctgggcgtgatctggggaagcgagactacctattacaactccgccctgaagtccagactgaccatcatcaaggataacagcaagagccaggtgttcctgaagatgaacagcctgcagaccgacgacaccgccatctactactgcgccaagcactactactacggcggctcctacgccatggactactggggccagggcacaagcgtgactgtgtctagcgccgccgcaatcgaggtgatgtacccccctccctacctggacaacgagaaaagcaacggcaccattatccatgtcaaaggcaagcacctgtgcccttcccccctgttccccggccccagcaagcccttctgggtgctggtggtggtgggcggcgtgctggcctgttacagcctgctggtgaccgtggccttcatcatcttctgggtgaggagcaagcggtccagactgctgcacagcgactacatgaacatgacaccaagaagaccaggccctaccagaaagcactaccagccctatgcccctcccagggacttcgccgcctatagatctcgggtgaagttctcaagaagcgccgacgcccctgcctaccagcagggccagaaccagctgtataacgaactgaacctgggccggagggaggagtacgatgtgctggacaagagaaggggcagggacccagagatggggggaaagcctcggaggaagaatccccaggagggcctgtataacgagctgcagaaggacaagatggccgaggcctacagcgagattggcatgaagggcgagagacggcgcggcaaggggcacgacggcctgtaccagggcctgtctaccgccacaaaggacacatacgacgccctgcatatgcaggccctgccacccaggtaataataa

SEQ ID NO: 512-513 - skipped (2 entries)

SEQ ID NO: 514 - protein, 107 aa, synthetic construct
EIVLTQTPSSLSASLGDRVTISCRASQSIGSSLHWYQQKPDQSPKLLIKYASQSFSGVPSKFIGSGSGTDFTLTINSLEAEDAATFYCHQSSTLPYTFGQGTKLEIK

SEQ ID NO: 515 - protein, 21 aa, synthetic construct
MALPVTALLLPLALLLHAARP

SEQ ID NO: 516 - DNA, 140 bp, synthetic construct
  source 1..140: mol_type = other DNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 517 - DNA, 100 bp, synthetic construct
  source 1..100: mol_type = other DNA
cgggtggcatccctgtgacccctccccagtgcctctcctggccctggaagttgccactccagtgcccaccagccttgtcctaataaaattaagttgcatc

SEQ ID NO: 518 - DNA, 48 bp, synthetic construct
  source 1..48: mol_type = other DNA
gggtcaagattagagaacggtcgtagcattatcggaggttctgccacc

SEQ ID NO: 519 - DNA, 59 bp, synthetic construct
  source 1..59: mol_type = other DNA
ggcacatttgaataaattctattaccagttcaagcttggcgtaatcatggtcatagctg

SEQ ID NO: 520 - protein, 22 aa, synthetic construct
MDMRVPAQLLGLLLLWLPGAKC

SEQ ID NO: 521 - protein, 93 aa, synthetic construct
LSNSIMYFSHFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 522 - protein, 66 aa, synthetic construct
IEVMYPPPYLDNERSNGTIIHVKGKHLCPSPLFPGPSKPFWVLVVVGGVLACYSLLVTVAFIIFWV

SEQ ID NO: 523-537 - skipped (15 entries)

SEQ ID NO: 538 - protein, 73 aa, synthetic construct
TTTPAPRPPTPAPTIASQPLSLRPEACRPAAGGAVHTRGLDFACDIYIWAPLAGTCGVLLLSLVITLYCNHRN

SEQ ID NO: 539 - protein, 53 aa, synthetic construct
SLRPEACRPAAGGAVHTRGLDFACDIYIWAPLAGTCGVLLLSLVITLYCNHRN

SEQ ID NO: 540 - protein, 93 aa, synthetic construct; identical to SEQ ID NO: 521

SEQ ID NO: 541 - protein, 40 aa, synthetic construct; identical to SEQ ID NO: 438

SEQ ID NO: 542 - protein, 26 aa, synthetic construct
WVLVVVGGVLACYSLLVTVAFIIFWV

SEQ ID NO: 543 - protein, 41 aa, synthetic construct
RSKRSRLLHSDYMNMTPRRPGPTRKHYQPYAPPRDFAAYRS

SEQ ID NO: 544 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 545-564 - skipped (20 entries)

SEQ ID NO: 565 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 566 - protein, 10 aa, synthetic construct
GFTFRSFGMS

SEQ ID NO: 567 - protein, 10 aa, synthetic construct
SISGSGSDTL

SEQ ID NO: 568 - protein, 6 aa, synthetic construct
GGSLSR

SEQ ID NO: 569 - protein, 98 aa, synthetic construct
QVQLVESGGGLVQPGGSLRLSCAASGFTLDYYAIGWFRQAPGKEREGVSCISSSDGSTYYADSVKGRFTISRDNAKNTVYLQMNSLKPEDTAVYYCAA

SEQ ID NO: 570 - RNA, 1110 nt, synthetic construct
  source 1..1110: mol_type = other RNA
atggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcctatttctaagcgactacacgctagcctggttcaggcaggcccccggcaaggagcgcgagtttgtgatcgttagcacgtggaaccgctatacctactacgctgaaggagtgaagggcaggttcacaatttctcgggacaacgccaagaaaaccgtgtatctgcagatgaactccctgcgccccgaggacacagccctgtactactgcgccgcaaaatttcgcatctcatacaacgttagggacgacagcttctacgacctgtggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctgggttcggtccaagcgctcacgcctgctacactcagactacatgaacatgacgccccgcagacctggacctaccaggaaacactatcaaccatatgcccccccaagggactttgcagcatacaggagccgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataa

SEQ ID NO: 571 - RNA, 1110 nt, synthetic construct
  source 1..1110: mol_type = other RNA
atggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcctatttctaagcgactacacgctagcctggttcaggcaggcccccggcaaggagcgcgagtttgtgatcgttagcacgtggaaccgctatacctactacgctgaaggagtgaagggcaggttcacaatttctcgggacaacgccaagaaaaccgtgtatctgcagatgaactccctgcgccccgaggacacagccctgtactactgcgccgcaaaatttcgcatctcatacaacgttagggacgacagcttctacgacctgtggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctggaagcggggacgcaagaagctgctgtatatctttaagcaaccattcatgcggcccgttcaaacgactcaagaagaagacgggtgtagctgccggtttcccgaagaagaagagggcggctgtgaactacgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataa

SEQ ID NO: 572 - RNA, 1122 nt, synthetic construct
  source 1..1122: mol_type = other RNA
atggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcgacaccttcggaacgaaagccatgggatggttcaggcaggcccccggcaagggacgcgagtttgtggccgcaatcacgtggacagcaggcctaacctactacgctgactccgtgaagggcaggttcacaatttctaaagaccgcgccaagaacaccgtggatctgcagatgaactccctgcgccccgaggacacagccctgtactactgcgcctcacggcgcacgttctcatacgagcaccccaattactataccaggtcagcctaccacttttggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctggaagcggggacgcaagaagctgctgtatatctttaagcaaccattcatgcggcccgttcaaacgactcaagaagaagacgggtgtagctgccggtttcccgaagaagaagagggcggctgtgaactacgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataa

SEQ ID NO: 573 - RNA, 1350 nt, synthetic construct
  source 1..1350: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcctatttctaagcgactacacgctagcctggttcaggcaggcccccggcaaggagcgcgagtttgtgatcgttagcacgtggaaccgctatacctactacgctgaaggagtgaagggcaggttcacaatttctcgggacaacgccaagaaaaccgtgtatctgcagatgaactccctgcgccccgaggacacagccctgtactactgcgccgcaaaatttcgcatctcatacaacgttagggacgacagcttctacgacctgtggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctgggttcggtccaagcgctcacgcctgctacactcagactacatgaacatgacgccccgcagacctggacctaccaggaaacactatcaaccatatgcccccccaagggactttgcagcatacaggagccgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataacgggtggcatccctgtgacccctccccagtgcctctcctggccctggaagttgccactccagtgcccaccagccttgtcctaataaaattaagttgcatc

SEQ ID NO: 574 - RNA, 1350 nt, synthetic construct
  source 1..1350: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcctatttctaagcgactacacgctagcctggttcaggcaggcccccggcaaggagcgcgagtttgtgatcgttagcacgtggaaccgctatacctactacgctgaaggagtgaagggcaggttcacaatttctcgggacaacgccaagaaaaccgtgtatctgcagatgaactccctgcgccccgaggacacagccctgtactactgcgccgcaaaatttcgcatctcatacaacgttagggacgacagcttctacgacctgtggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctggaagcggggacgcaagaagctgctgtatatctttaagcaaccattcatgcggcccgttcaaacgactcaagaagaagacgggtgtagctgccggtttcccgaagaagaagagggcggctgtgaactacgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataacgggtggcatccctgtgacccctccccagtgcctctcctggccctggaagttgccactccagtgcccaccagccttgtcctaataaaattaagttgcatc

SEQ ID NO: 575 - RNA, 1362 nt, synthetic construct
  source 1..1362: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggctctgcccgtgaccgcactgctgctgcccctggccctgctgctgcacgccgccaggcccgaggtgcagctggtggagtccggcggcggcgtggtgcagccaggcggctctctgaggctgagctgcgccgccagcggcgacaccttcggaacgaaagccatgggatggttcaggcaggcccccggcaagggacgcgagtttgtggccgcaatcacgtggacagcaggcctaacctactacgctgactccgtgaagggcaggttcacaatttctaaagaccgcgccaagaacaccgtggatctgcagatgaactccctgcgccccgaggacacagccctgtactactgcgcctcacggcgcacgttctcatacgagcaccccaattactataccaggtcagcctaccacttttggggacagggcaccctggtgaccgtgtcctccgccgccgctatcgaggtgatgtaccctcccccttacctggacaacgagaaatcaaacggcaccatcatccacgtgaagggaaagcacctgtgtcctagcccactgttcccaggccccagcaagcccttttgggtgctggttgtggtgggcggcgtgctggcctgctacagcctgctggtcaccgtggccttcatcatcttctggaagcggggacgcaagaagctgctgtatatctttaagcaaccattcatgcggcccgttcaaacgactcaagaagaagacgggtgtagctgccggtttcccgaagaagaagagggcggctgtgaactacgcgtgaagttctccaggagcgccgatgcccccgcctaccagcagggccagaaccagctgtacaacgagctgaacctgggccggcgggaggaatacgacgtgctggacaagcggagaggccgggaccctgagatgggcggaaagcctagacgcaagaacccccaggagggcctgtacaacgagctgcagaaagataaaatggccgaggcctatagcgagatcggcatgaagggcgagagaagacgcggcaaggggcacgatggcctgtaccaggggctgagcaccgccaccaaagacacctacgatgccctgcacatgcaggccctgcccccgcggtaataataacgggtggcatccctgtgacccctccccagtgcctctcctggccctggaagttgccactccagtgcccaccagccttgtcctaataaaattaagttgcatc

SEQ ID NO: 576 - protein, 484 aa, synthetic construct
METDTLLLWVLLLWVPGSTGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 577 - protein, 491 aa, synthetic construct
METDTLLLWVLLLWVPGSTGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKASTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 578 - protein, 475 aa, synthetic construct
METDTLLLWVLLLWVPGSTGQVQLVESGGGVVQPGRSLRLSCAASGFTFSSYGMHWVRQAPGKGLEWVAVIWYDGSNKYYVDSVKGRFTISRDNSKNTMYLQMNSLRAEDTAVYYCARRGYNWNIAFDIWGQGTMVTVSSGGGGSGGGGSGGGGSGGGGSDIQMTQSPSTLSASVGDRVTITCRASQSISSWLAWYQQKPGKAPKLLIYKASSLENGVPSRFSGSGSGTEFTLTISSLQPDDFATYYCQQYNSYWTFGQGTKVEIKASSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 579 - protein, 502 aa, synthetic construct
METDTLLLWVLLLWVPGSTGQVQLVQSGAEVKKPGASVKVSCKASGYTFTSYGISWVRQAPGQGLEWMGWISAYNGNTNYAQKLQDRVTMTTHTSTSAAYMELRSLRSDDTAVYYCARDGGLVAASPDYWGQGTLVTVSSGGGGSGGGGSGGGGSGGGGSDIVMTQSPDSLAVSLGERATINCKSSQSVLYNSNNKNYLAWYQQKPGQPPKLLIYRASTRDSGVPDRFSGSGSGTDFTLTISSLQAEDVALYYCQQYYSTPYTFGQGTKLEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 580 - protein, 392 aa, synthetic construct
METDTLLLWVLLLWVPGSTGEVQLVESGGGLVQAGESLRLSCIASGRTMSSYAMAWFRQAPGKEREFVAVSSLSGGTSDYADSVKGRFIISRDNARSTVTLQMNNLKPEDTAVYYCAARPVYIRYSIRDYDIWGQGTLVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 581 - protein, 372 aa, synthetic construct
METDTLLLWVLLLWVPGSTGEVQLVESGGGLVQAGESLRLSCIASGRTMSSYAMAWFRQAPGKEREFVAVSSLSGGTSDYADSVKGRFIISRDNARSTVTLQMNNLKPEDTAVYYCAARPVYIRYSIRDYDIWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 582 - protein, 392 aa, synthetic construct
METDTLLLWVLLLWVPGSTGEVQLVESGGGLVQAGGSLRLSCAFSGRTFSSYAMGWFRQAPGKEREFVATISLSGGTTWYADSVKGRFTISRDNGASTAYLQMNSLKPEDTAVYYCAARPVYHKTSARDYDFWGQGTQVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 583 - protein, 372 aa, synthetic construct
METDTLLLWVLLLWVPGSTGEVQLVESGGGLVQAGGSLRLSCAFSGRTFSSYAMGWFRQAPGKEREFVATISLSGGTTWYADSVKGRFTISRDNGASTAYLQMNSLKPEDTAVYYCAARPVYHKTSARDYDFWGQGTQVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 584 - protein, 392 aa, synthetic construct
METDTLLLWVLLLWVPGSTGEVQLVESGGGLVQAGGSLRLSCAASGFTFSSYAMAWFRQAPGKEREFVAVISLSGGSVDYRDSVKGRFTISRDNAQSTVSLQMNNLKPEDTAIYYCAARPVYMKWSERDFDYWGQGTLVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 585 - protein, 372 aa, synthetic construct
METDTLLLWVLLLWVPGSTGEVQLVESGGGLVQAGGSLRLSCAASGFTFSSYAMAWFRQAPGKEREFVAVISLSGGSVDYRDSVKGRFTISRDNAQSTVSLQMNNLKPEDTAIYYCAARPVYMKWSERDFDYWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 586 - protein, 364 aa, synthetic construct
METDTLLLWVLLLWVPGSTGEVQLVESGGGLVQAGGSLKLACAASVHTSYTYGMGWFRQAPGKEREFVAVISSDGVRTNYADSVQGRFTISRDNAKNTIYLQMNNLKTEDSAIYYCAAGSGLFSAGYEYQYWGQGTQVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 587 - protein, 371 aa, synthetic construct
METDTLLLWVLLLWVPGSTGEVQLVESGGGLVQAGGSLKLACAASVHTSYTYGMGWFRQAPGKEREFVAVISSDGVRTNYADSVQGRFTISRDNAKNTIYLQMNNLKTEDSAIYYCAAGSGLFSAGYEYQYWGQGTQVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 588 - protein, 393 aa, synthetic construct
METDTLLLWVLLLWVPGSTGEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 589 - protein, 373 aa, synthetic construct
METDTLLLWVLLLWVPGSTGEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 590 - protein, 505 aa, synthetic construct
METDTLLLWVLLLWVPGSTGEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSGGGGSGGGGSGGGGSEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 591 - protein, 509 aa, synthetic construct
METDTLLLWVLLLWVPGSTGEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSGGGGSGGGGSGGGGSEVQLVESGGGLVQPGGSLSLSCAASGDTFGTKAMGWFRQAPGKGREFVAAITWTAGLTYLADSAKGRFTISKDRVQNTVYLEMNNLEPEDTAVYYCASRRTFSYEHPNYYTASAYHFWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 592 - protein, 504 aa, synthetic construct
METDTLLLWVLLLWVPGSTGEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSGGGGSGGGGSGGGGSEVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 593 - protein, 352 aa, synthetic construct
METDTLLLWVLLLWVPGSTGEVQLVESGGGLVQAGGSLRLSCAGSGFTFSSTVIGWYRQAPGKQREFVALITAGGHTNSVEDRFIISRDNAKSTAYLQMTSLKSEDTAVYYCNLPNQGYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 594 - protein, 360 aa, synthetic construct
METDTLLLWVLLLWVPGSTGEVQLVESGGGLVQPGGSLRLFCAASGSRLSFTTMGWYRQAPGKQREVVAYITESGSTAYTASVKDRFTISRDNAKNTVSLQMNSLKPEDTAVYFCNAVIMMGGSGTYWGPGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 595 - protein, 366 aa, synthetic construct
METDTLLLWVLLLWVPGSTGEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 596 - protein, 393 aa, synthetic construct
METDTLLLWVLLLWVPGSTGEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 597 - protein, 505 aa, synthetic construct
METDTLLLWVLLLWVPGSTGEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSGGGGSGGGGSGGGGSEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 598 - protein, 504 aa, synthetic construct
METDTLLLWVLLLWVPGSTGEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSGGGGSGGGGSGGGGSEVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 599 - protein, 393 aa, synthetic construct
METDTLLLWVLLLWVPGSTGEVQLVESGGGLVPPGGSLRLSCAASGRTGDINGMGWFRRAAGKEREFVAATRWNGGTKYYTSSVEGRFTISGDRVGNTVTLEMNNLVPEDTARYYCATTRRRFDFYSVGGYENWGQGTQVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 600 - protein, 373 aa, synthetic construct
METDTLLLWVLLLWVPGSTGEVQLVESGGGLVPPGGSLRLSCAASGRTGDINGMGWFRRAAGKEREFVAATRWNGGTKYYTSSVEGRFTISGDRVGNTVTLEMNNLVPEDTARYYCATTRRRFDFYSVGGYENWGQGTQVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 601 - protein, 370 aa, synthetic construct
METDTLLLWVLLLWVPGSTGEVQLVESGGGLVQPGGSLSLSCAASGDTFGTKAMGWFRQAPGKGREFVAAITWTAGLTYLADSAKGRFTISKDRVQNTVYLEMNNLEPEDTAVYYCASRRTFSYEHPNYYTASAYHFWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 602 - protein, 509 aa, synthetic construct
METDTLLLWVLLLWVPGSTGEVQLVESGGGLVQPGGSLSLSCAASGDTFGTKAMGWFRQAPGKGREFVAAITWTAGLTYLADSAKGRFTISKDRVQNTVYLEMNNLEPEDTAVYYCASRRTFSYEHPNYYTASAYHFWGQGTLVTVSSGGGGSGGGGSGGGGSEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 603 - protein, 366 aa, synthetic construct
METDTLLLWVLLLWVPGSTGEVQLVESGGGLVQPGGSLRLSCVASGLFLSDYTLAWFRQAPGKEREFVIVSTWNGYTYYGESVSGRFTISRDNAEKTVFLQMNSLKPEDTAVYYCAAKFRISYNVRDDSFYDLWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 604 - protein, 373 aa, synthetic construct
METDTLLLWVLLLWVPGSTGEVQLVESGGGLVQPGGSLRLSCVASGLFLSDYTLAWFRQAPGKEREFVIVSTWNGYTYYGESVSGRFTISRDNAEKTVFLQMNSLKPEDTAVYYCAAKFRISYNVRDDSFYDLWGQGTLVTVSSASTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 605 - protein, 373 aa, synthetic construct
METDTLLLWVLLLWVPGSTGEVQLVESGGGLVQPGGSLRLSCVASGLFLSDYTLAWFRQAPGKEREFVIVSTWNGYTYYGESVSGRFTISRDNAEKTVFLQMNSLKPEDTAVYYCAAKFRISYNVRDDSFYDLWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 606 - protein, 373 aa, synthetic construct
METDTLLLWVLLLWVPGSTGEVQLVESGGGLVQAGGSLTLTCAASGLTFAGYSVGWFRQAPGKEREFVAVITRGGDYRYFAESVQGRFTVSRDNGKNTVSLQMLDLKPDDTAVYYCAAGTMTYAIRETRAYNYWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 607 - protein, 365 aa, synthetic construct
METDTLLLWVLLLWVPGSTGEVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 608 - protein, 504 aa, synthetic construct
METDTLLLWVLLLWVPGSTGEVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSGGGGSGGGGSGGGGSEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 609 - protein, 504 aa, synthetic construct
METDTLLLWVLLLWVPGSTGEVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSGGGGSGGGGSGGGGSEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 610 - protein, 365 aa, synthetic construct
METDTLLLWVLLLWVPGSTGEVQLVESGGGQVQAGGSLRLSCVHSGRTFSSYVAAWFRQYPGKEREFVAAASLSGGTTDYADSVKGRFTITRDNAASTHYLQMNSLQPEDTAVYYCAARPVYLKYTYHDYDFWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 611 - protein, 372 aa, synthetic construct
METDTLLLWVLLLWVPGSTGEVQLVESGGGLVQAGDSLRLSCSLSGRTFSSYAMAWFRQLPGKEREFVSVISLSGQNTYYADSVKGRFTISRDNSKSTVFLQMNSLKPEDTAVYYCAARPVYHKFHYRDYDFWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 612 - protein, 354 aa, synthetic construct
METDTLLLWVLLLWVPGSTGEVQLVESGGGLVQPGGSMRLSCQGSGRSFRLNTMVWYRRASGNQRELVASLTSGGNTYYADSVKGRFTISRDDAKSTVDLQMNSLRPADTAVYFCYMGGNSWGQETQVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 613 - protein, 367 aa, synthetic construct; identical to SEQ ID NO: 49

SEQ ID NO: 614 - protein, 367 aa, synthetic construct; identical to SEQ ID NO: 50

SEQ ID NO: 615 - protein, 371 aa, synthetic construct
MALPVTALLLPLALLLHAARPEVQLVESGGGVVQPGGSLRLSCAASGDTFGTKAMGWFRQAPGKGREFVAAITWTAGLTYYADSVKGRFTISKDRAKNTVDLQMNSLRPEDTALYYCASRRTFSYEHPNYYTRSAYHFWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 616 - protein, 371 aa, synthetic construct; identical to SEQ ID NO: 51

SEQ ID NO: 617 - protein, 366 aa, synthetic construct
MALPVTALLLPLALLLHAARPEVQLVESGGGVVQPGGSLRLSCAASGFTFSSYAMAWFRQAPGKEREFVAVISLSGGSVDYRDSVKGRFTISRDNAKNTVDLQMNSLRPEDTALYYCAARPVYIKWSERDFDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 618 - protein, 366 aa, synthetic construct
MALPVTALLLPLALLLHAARPEVQLVESGGGVVQPGGSLRLSCAASGFTFSSYAMAWFRQAPGKEREFVAVISLSGGSVDYRDSVKGRFTISRDNAKNTVDLQMNSLRPEDTALYYCAARPVYIKWSERDFDYWGQGTLVTVSSAAAIEVMYPPPYLDNEKSNGTIIHVKGKHLCPSPLFPGPSKPFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 619 - protein, 371 aa, synthetic construct; identical to SEQ ID NO: 615

SEQ ID NO: 620 - protein, 366 aa, synthetic construct; identical to SEQ ID NO: 617

SEQ ID NO: 621 - protein, 366 aa, synthetic construct; identical to SEQ ID NO: 618

SEQ ID NO: 622 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 623 - protein, 489 aa, synthetic construct; identical to SEQ ID NO: 622

SEQ ID NO: 624 - protein, 464 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 625 - protein, 471 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKASTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 626 - protein, 455 aa, synthetic construct
QVQLVESGGGVVQPGRSLRLSCAASGFTFSSYGMHWVRQAPGKGLEWVAVIWYDGSNKYYVDSVKGRFTISRDNSKNTMYLQMNSLRAEDTAVYYCARRGYNWNIAFDIWGQGTMVTVSSGGGGSGGGGSGGGGSGGGGSDIQMTQSPSTLSASVGDRVTITCRASQSISSWLAWYQQKPGKAPKLLIYKASSLENGVPSRFSGSGSGTEFTLTISSLQPDDFATYYCQQYNSYWTFGQGTKVEIKASSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 627 - protein, 482 aa, synthetic construct
QVQLVQSGAEVKKPGASVKVSCKASGYTFTSYGISWVRQAPGQGLEWMGWISAYNGNTNYAQKLQDRVTMTTHTSTSAAYMELRSLRSDDTAVYYCARDGGLVAASPDYWGQGTLVTVSSGGGGSGGGGSGGGGSGGGGSDIVMTQSPDSLAVSLGERATINCKSSQSVLYNSNNKNYLAWYQQKPGQPPKLLIYRASTRDSGVPDRFSGSGSGTDFTLTISSLQAEDVALYYCQQYYSTPYTFGQGTKLEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 628 - protein, 372 aa, synthetic construct
EVQLVESGGGLVQAGESLRLSCIASGRTMSSYAMAWFRQAPGKEREFVAVSSLSGGTSDYADSVKGRFIISRDNARSTVTLQMNNLKPEDTAVYYCAARPVYIRYSIRDYDIWGQGTLVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 629 - protein, 352 aa, synthetic construct
EVQLVESGGGLVQAGESLRLSCIASGRTMSSYAMAWFRQAPGKEREFVAVSSLSGGTSDYADSVKGRFIISRDNARSTVTLQMNNLKPEDTAVYYCAARPVYIRYSIRDYDIWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 630 - protein, 372 aa, synthetic construct
EVQLVESGGGLVQAGGSLRLSCAFSGRTFSSYAMGWFRQAPGKEREFVATISLSGGTTWYADSVKGRFTISRDNGASTAYLQMNSLKPEDTAVYYCAARPVYHKTSARDYDFWGQGTQVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 631 - protein, 352 aa, synthetic construct
EVQLVESGGGLVQAGGSLRLSCAFSGRTFSSYAMGWFRQAPGKEREFVATISLSGGTTWYADSVKGRFTISRDNGASTAYLQMNSLKPEDTAVYYCAARPVYHKTSARDYDFWGQGTQVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 632 - protein, 372 aa, synthetic construct
EVQLVESGGGLVQAGGSLRLSCAASGFTFSSYAMAWFRQAPGKEREFVAVISLSGGSVDYRDSVKGRFTISRDNAQSTVSLQMNNLKPEDTAIYYCAARPVYMKWSERDFDYWGQGTLVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 633 - protein, 352 aa, synthetic construct
EVQLVESGGGLVQAGGSLRLSCAASGFTFSSYAMAWFRQAPGKEREFVAVISLSGGSVDYRDSVKGRFTISRDNAQSTVSLQMNNLKPEDTAIYYCAARPVYMKWSERDFDYWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 634 - protein, 344 aa, synthetic construct
EVQLVESGGGLVQAGGSLKLACAASVHTSYTYGMGWFRQAPGKEREFVAVISSDGVRTNYADSVQGRFTISRDNAKNTIYLQMNNLKTEDSAIYYCAAGSGLFSAGYEYQYWGQGTQVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 635 - protein, 351 aa, synthetic construct
EVQLVESGGGLVQAGGSLKLACAASVHTSYTYGMGWFRQAPGKEREFVAVISSDGVRTNYADSVQGRFTISRDNAKNTIYLQMNNLKTEDSAIYYCAAGSGLFSAGYEYQYWGQGTQVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 636 - protein, 373 aa, synthetic construct
EVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 637 - protein, 353 aa, synthetic construct
EVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 638 - protein, 485 aa, synthetic construct
EVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSGGGGSGGGGSGGGGSEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 639 - protein, 489 aa, synthetic construct
EVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSGGGGSGGGGSGGGGSEVQLVESGGGLVQPGGSLSLSCAASGDTFGTKAMGWFRQAPGKGREFVAAITWTAGLTYLADSAKGRFTISKDRVQNTVYLEMNNLEPEDTAVYYCASRRTFSYEHPNYYTASAYHFWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 640 - protein, 484 aa, synthetic construct
EVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSGGGGSGGGGSGGGGSEVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 641 - protein, 332 aa, synthetic construct
EVQLVESGGGLVQAGGSLRLSCAGSGFTFSSTVIGWYRQAPGKQREFVALITAGGHTNSVEDRFIISRDNAKSTAYLQMTSLKSEDTAVYYCNLPNQGYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 642 - protein, 340 aa, synthetic construct
EVQLVESGGGLVQPGGSLRLFCAASGSRLSFTTMGWYRQAPGKQREVVAYITESGSTAYTASVKDRFTISRDNAKNTVSLQMNSLKPEDTAVYFCNAVIMMGGSGTYWGPGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 643 - protein, 346 aa, synthetic construct
EVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 644 - protein, 373 aa, synthetic construct
EVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 645 - protein, 485 aa, synthetic construct
EVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSGGGGSGGGGSGGGGSEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 646 - protein, 484 aa, synthetic construct
EVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSGGGGSGGGGSGGGGSEVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 647 - protein, 373 aa, synthetic construct
EVQLVESGGGLVPPGGSLRLSCAASGRTGDINGMGWFRRAAGKEREFVAATRWNGGTKYYTSSVEGRFTISGDRVGNTVTLEMNNLVPEDTARYYCATTRRRFDFYSVGGYENWGQGTQVTVSSASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 648 - protein, 353 aa, synthetic construct
EVQLVESGGGLVPPGGSLRLSCAASGRTGDINGMGWFRRAAGKEREFVAATRWNGGTKYYTSSVEGRFTISGDRVGNTVTLEMNNLVPEDTARYYCATTRRRFDFYSVGGYENWGQGTQVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 649 - protein, 350 aa, synthetic construct
EVQLVESGGGLVQPGGSLSLSCAASGDTFGTKAMGWFRQAPGKGREFVAAITWTAGLTYLADSAKGRFTISKDRVQNTVYLEMNNLEPEDTAVYYCASRRTFSYEHPNYYTASAYHFWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 650 - protein, 489 aa, synthetic construct
EVQLVESGGGLVQPGGSLSLSCAASGDTFGTKAMGWFRQAPGKGREFVAAITWTAGLTYLADSAKGRFTISKDRVQNTVYLEMNNLEPEDTAVYYCASRRTFSYEHPNYYTASAYHFWGQGTLVTVSSGGGGSGGGGSGGGGSEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 651 - protein, 346 aa, synthetic construct
EVQLVESGGGLVQPGGSLRLSCVASGLFLSDYTLAWFRQAPGKEREFVIVSTWNGYTYYGESVSGRFTISRDNAEKTVFLQMNSLKPEDTAVYYCAAKFRISYNVRDDSFYDLWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 652 - protein, 353 aa, synthetic construct
EVQLVESGGGLVQPGGSLRLSCVASGLFLSDYTLAWFRQAPGKEREFVIVSTWNGYTYYGESVSGRFTISRDNAEKTVFLQMNSLKPEDTAVYYCAAKFRISYNVRDDSFYDLWGQGTLVTVSSASTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 653 - protein, 353 aa, synthetic construct
EVQLVESGGGLVQPGGSLRLSCVASGLFLSDYTLAWFRQAPGKEREFVIVSTWNGYTYYGESVSGRFTISRDNAEKTVFLQMNSLKPEDTAVYYCAAKFRISYNVRDDSFYDLWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 654 - protein, 353 aa, synthetic construct
EVQLVESGGGLVQAGGSLTLTCAASGLTFAGYSVGWFRQAPGKEREFVAVITRGGDYRYFAESVQGRFTVSRDNGKNTVSLQMLDLKPDDTAVYYCAAGTMTYAIRETRAYNYWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 655 - protein, 345 aa, synthetic construct
EVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 656-665 - skipped (10 entries)

SEQ ID NO: 666 - protein, 484 aa, synthetic construct
EVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSGGGGSGGGGSGGGGSEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 667 - protein, 484 aa, synthetic construct
EVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSGGGGSGGGGSGGGGSEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 668 - protein, 345 aa, synthetic construct
EVQLVESGGGQVQAGGSLRLSCVHSGRTFSSYVAAWFRQYPGKEREFVAAASLSGGTTDYADSVKGRFTITRDNAASTHYLQMNSLQPEDTAVYYCAARPVYLKYTYHDYDFWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 669 - protein, 352 aa, synthetic construct
EVQLVESGGGLVQAGDSLRLSCSLSGRTFSSYAMAWFRQLPGKEREFVSVISLSGQNTYYADSVKGRFTISRDNSKSTVFLQMNSLKPEDTAVYYCAARPVYHKFHYRDYDFWGQGTLVTVSSASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 670 - protein, 334 aa, synthetic construct
EVQLVESGGGLVQPGGSMRLSCQGSGRSFRLNTMVWYRRASGNQRELVASLTSGGNTYYADSVKGRFTISRDDAKSTVDLQMNSLRPADTAVYFCYMGGNSWGQETQVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 671 - protein, 346 aa, synthetic construct; identical to SEQ ID NO: 435

SEQ ID NO: 672 - protein, 346 aa, synthetic construct; identical to SEQ ID NO: 436

SEQ ID NO: 673 - protein, 350 aa, synthetic construct
EVQLVESGGGVVQPGGSLRLSCAASGDTFGTKAMGWFRQAPGKGREFVAAITWTAGLTYYADSVKGRFTISKDRAKNTVDLQMNSLRPEDTALYYCASRRTFSYEHPNYYTRSAYHFWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 674 - protein, 350 aa, synthetic construct; identical to SEQ ID NO: 437

SEQ ID NO: 675 - protein, 345 aa, synthetic construct
EVQLVESGGGVVQPGGSLRLSCAASGFTFSSYAMAWFRQAPGKEREFVAVISLSGGSVDYRDSVKGRFTISRDNAKNTVDLQMNSLRPEDTALYYCAARPVYIKWSERDFDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 676 - protein, 345 aa, synthetic construct
EVQLVESGGGVVQPGGSLRLSCAASGFTFSSYAMAWFRQAPGKEREFVAVISLSGGSVDYRDSVKGRFTISRDNAKNTVDLQMNSLRPEDTALYYCAARPVYIKWSERDFDYWGQGTLVTVSSAAAIEVMYPPPYLDNEKSNGTIIHVKGKHLCPSPLFPGPSKPFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 677 - protein, 350 aa, synthetic construct; identical to SEQ ID NO: 673

SEQ ID NO: 678 - protein, 345 aa, synthetic construct; identical to SEQ ID NO: 675

SEQ ID NO: 679 - protein, 345 aa, synthetic construct; identical to SEQ ID NO: 676

SEQ ID NO: 680 - protein, 467 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 681 - protein, 467 aa, synthetic construct; identical to SEQ ID NO: 680

SEQ ID NO: 682-683 - skipped (2 entries)

SEQ ID NO: 684 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 685 - protein, 120 aa, synthetic construct
QVQLVESGGGVVQPGRSLRLSCAASGFTFSSYGMHWVRQAPGKGLEWVAVIWYDGSNKYYVDSVKGRFTISRDNSKNTMYLQMNSLRAEDTAVYYCARRGYNWNIAFDIWGQGTMVTVSS

SEQ ID NO: 686 - protein, 120 aa, synthetic construct
QVQLVQSGAEVKKPGASVKVSCKASGYTFTSYGISWVRQAPGQGLEWMGWISAYNGNTNYAQKLQDRVTMTTHTSTSAAYMELRSLRSDDTAVYYCARDGGLVAASPDYWGQGTLVTVSS

SEQ ID NO: 687 - protein, 123 aa, synthetic construct
EVQLVESGGGLVQAGESLRLSCIASGRTMSSYAMAWFRQAPGKEREFVAVSSLSGGTSDYADSVKGRFIISRDNARSTVTLQMNNLKPEDTAVYYCAARPVYIRYSIRDYDIWGQGTLVTVSS

SEQ ID NO: 688 - protein, 123 aa, synthetic construct
EVQLVESGGGLVQAGGSLRLSCAFSGRTFSSYAMGWFRQAPGKEREFVATISLSGGTTWYADSVKGRFTISRDNGASTAYLQMNSLKPEDTAVYYCAARPVYHKTSARDYDFWGQGTQVTVSS

SEQ ID NO: 689-698 - skipped (10 entries)

SEQ ID NO: 699 - protein, 123 aa, synthetic construct
EVQLVESGGGLVQAGGSLRLSCAASGFTFSSYAMAWFRQAPGKEREFVAVISLSGGSVDYRDSVKGRFTISRDNAQSTVSLQMNNLKPEDTAIYYCAARPVYMKWSERDFDYWGQGTLVTVSS

SEQ ID NO: 700 - protein, 122 aa, synthetic construct
EVQLVESGGGLVQAGGSLKLACAASVHTSYTYGMGWFRQAPGKEREFVAVISSDGVRTNYADSVQGRFTISRDNAKNTIYLQMNNLKTEDSAIYYCAAGSGLFSAGYEYQYWGQGTQVTVSS

SEQ ID NO: 701 - protein, 124 aa, synthetic construct
EVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSS

SEQ ID NO: 702 - protein, 263 aa, synthetic construct
EVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSGGGGSGGGGSGGGGSEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSS

SEQ ID NO: 703 - protein, 267 aa, synthetic construct
EVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSGGGGSGGGGSGGGGSEVQLVESGGGLVQPGGSLSLSCAASGDTFGTKAMGWFRQAPGKGREFVAAITWTAGLTYLADSAKGRFTISKDRVQNTVYLEMNNLEPEDTAVYYCASRRTFSYEHPNYYTASAYHFWGQGTLVTVSS

SEQ ID NO: 704 - protein, 262 aa, synthetic construct
EVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSSGGGGSGGGGSGGGGSEVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSS

SEQ ID NO: 705 - protein, 110 aa, synthetic construct
EVQLVESGGGLVQAGGSLRLSCAGSGFTFSSTVIGWYRQAPGKQREFVALITAGGHTNSVEDRFIISRDNAKSTAYLQMTSLKSEDTAVYYCNLPNQGYWGQGTLVTVSS

SEQ ID NO: 706 - protein, 118 aa, synthetic construct
EVQLVESGGGLVQPGGSLRLFCAASGSRLSFTTMGWYRQAPGKQREVVAYITESGSTAYTASVKDRFTISRDNAKNTVSLQMNSLKPEDTAVYFCNAVIMMGGSGTYWGPGTLVTVSS

SEQ ID NO: 707 - protein, 124 aa, synthetic construct
EVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSS

SEQ ID NO: 708 - protein, 263 aa, synthetic construct
EVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSGGGGSGGGGSGGGGSEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSS

SEQ ID NO: 709 - protein, 262 aa, synthetic construct
EVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSSGGGGSGGGGSGGGGSEVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSS

SEQ ID NO: 710 - protein, 124 aa, synthetic construct
EVQLVESGGGLVPPGGSLRLSCAASGRTGDINGMGWFRRAAGKEREFVAATRWNGGTKYYTSSVEGRFTISGDRVGNTVTLEMNNLVPEDTARYYCATTRRRFDFYSVGGYENWGQGTQVTVSS

SEQ ID NO: 711 - protein, 128 aa, synthetic construct
EVQLVESGGGLVQPGGSLSLSCAASGDTFGTKAMGWFRQAPGKGREFVAAITWTAGLTYLADSAKGRFTISKDRVQNTVYLEMNNLEPEDTAVYYCASRRTFSYEHPNYYTASAYHFWGQGTLVTVSS

SEQ ID NO: 712 - protein, 267 aa, synthetic construct
EVQLVESGGGLVQPGGSLSLSCAASGDTFGTKAMGWFRQAPGKGREFVAAITWTAGLTYLADSAKGRFTISKDRVQNTVYLEMNNLEPEDTAVYYCASRRTFSYEHPNYYTASAYHFWGQGTLVTVSSGGGGSGGGGSGGGGSEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSS

SEQ ID NO: 713 - protein, 124 aa, synthetic construct
EVQLVESGGGLVQPGGSLRLSCVASGLFLSDYTLAWFRQAPGKEREFVIVSTWNGYTYYGESVSGRFTISRDNAEKTVFLQMNSLKPEDTAVYYCAAKFRISYNVRDDSFYDLWGQGTLVTVSS

SEQ ID NO: 714 - protein, 124 aa, synthetic construct
EVQLVESGGGLVQAGGSLTLTCAASGLTFAGYSVGWFRQAPGKEREFVAVITRGGDYRYFAESVQGRFTVSRDNGKNTVSLQMLDLKPDDTAVYYCAAGTMTYAIRETRAYNYWGQGTLVTVSS

SEQ ID NO: 715 - protein, 123 aa, synthetic construct
EVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSS

SEQ ID NO: 716 - protein, 262 aa, synthetic construct
EVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSGGGGSGGGGSGGGGSEVQLVESGGGLVQAEGSLRLSCTASGLTFDNYAMGWFRQAPGKEREFVATISAAGHRTIFTNSVRGRFTASRDNAKKTTYLQMNSLKPEDTAVYYCAASPSQYGSMGKSAYDYWGQGTLVTVSS

SEQ ID NO: 717 - protein, 262 aa, synthetic construct
EVQLVESGGGLVQTGDSLRLTCAASGRTIGDRSMAWFRQAPGKEREFVIVNTWNGYTYTDESVEGRFTISRDSGANTVHLQMNKLNPKDTAVYYCASSSYTYNIRQRSSYDWWGQGTLVTVSSGGGGSGGGGSGGGGSEVQLVESGGGLVQAGDSLRLSCAASGRTLSSYTTGWFRQGLGKEREFVAAITKNGPYLYYKDSVRGRFTISRDSAKTTVFLQMDNLKPEDTAVYYCAVSDAQYDIRNREKYYYWGQGTLVTVSS

SEQ ID NO: 718 - protein, 123 aa, synthetic construct
EVQLVESGGGQVQAGGSLRLSCVHSGRTFSSYVAAWFRQYPGKEREFVAAASLSGGTTDYADSVKGRFTITRDNAASTHYLQMNSLQPEDTAVYYCAARPVYLKYTYHDYDFWGQGTLVTVSS

SEQ ID NO: 719 - protein, 123 aa, synthetic construct
EVQLVESGGGLVQAGDSLRLSCSLSGRTFSSYAMAWFRQLPGKEREFVSVISLSGQNTYYADSVKGRFTISRDNSKSTVFLQMNSLKPEDTAVYYCAARPVYHKFHYRDYDFWGQGTLVTVSS

SEQ ID NO: 720 - protein, 112 aa, synthetic construct
EVQLVESGGGLVQPGGSMRLSCQGSGRSFRLNTMVWYRRASGNQRELVASLTSGGNTYYADSVKGRFTISRDDAKSTVDLQMNSLRPADTAVYFCYMGGNSWGQETQVTVSS

SEQ ID NO: 721 - protein, 123 aa, synthetic construct
EVQLVESGGGVVQPGGSLRLSCAASGFTFSSYAMAWFRQAPGKEREFVAVISLSGGSVDYRDSVKGRFTISRDNAKNTVDLQMNSLRPEDTALYYCAARPVYIKWSERDFDYWGQGTLVTVSS

SEQ ID NO: 722 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 723 - protein, 123 aa, synthetic construct
EVQLVESGGGVVQPGGSLRLSCAASGFTFSSYAMAWFRQAPGKEREFVAVISLSGGSVDYRDSVKGRFTISRDNAQSTVSLQMNNLRPEDTALYYCAARPVYMKWSERDFDYWGQGTLVTVSS

SEQ ID NO: 724 - protein, 123 aa, synthetic construct
EVQLVESGGGVVQPGGSLRLSCAASGFTFSSYAMAWFRQAPGKEREFVAVISLSGGSVDYADSVKGRFTISRDNAQSTVSLQMNSLRPEDTALYYCAARPVYMKWSERDFDYWGQGTLVTVSS

SEQ ID NO: 725 - protein, 123 aa, synthetic construct
EVQLVESGGGVVQPGGSLRLSCAASGFTFSSYAMAWFRQAPGKEREFVAVISLSGGSVDYRDSVKGRFTISRDNAKSTVSLQMNSLRPEDTALYYCAARPVYMKWSERDFDYWGQGTLVTVSS

SEQ ID NO: 726 - protein, 123 aa, synthetic construct
EVQLVESGGGVVQPGGSLRLSCAASGFTFSSYAMAWFRQAPGKEREFVAVISLSGGSVDYADSVKGRFTISRDNAKSTVSLQMNSLRPEDTALYYCAARPVYMKWSERDFDYWGQGTLVTVSS

SEQ ID NO: 727 - protein, 123 aa, synthetic construct
EVQLVESGGGVVQPGGSLRLSCAASGFTFSSYAMAWFRQAPGKEREFVAVISLSGGSVDYRDSVKGRFTISRDNAQNTVSLQMNSLRPEDTALYYCAARPVYMKWSERDFDYWGQGTLVTVSS

SEQ ID NO: 728 - protein, 123 aa, synthetic construct
EVQLVESGGGVVQPGGSLRLSCAASGFTFSSYAMAWFRQAPGKEREFVAVISLSGGSVDYADSVKGRFTISRDNAQNTVSLQMNSLRPEDTALYYCAARPVYMKWSERDFDYWGQGTLVTVSS

SEQ ID NO: 729 - protein, 123 aa, synthetic construct
EVQLVESGGGVVQPGGSLRLSCAASGFTFSSYAMAWFRQAPGKEREFVAVISLSGGSVDYRDSVKGRFTISRDNAKNTVSLQMNSLRPEDTALYYCAARPVYMKWSERDFDYWGQGTLVTVSS

SEQ ID NO: 730 - protein, 123 aa, synthetic construct
EVQLVESGGGVVQPGGSLRLSCAASGFTFSSYAMAWFRQAPGKEREFVAVISLSGGSVDYADSVKGRFTISRDNAKNTVSLQMNSLRPEDTALYYCAARPVYMKWSERDFDYWGQGTLVTVSS

SEQ ID NO: 731 - protein, 123 aa, synthetic construct
EVQLVESGGGVVQPGGSLRLSCAASGFTFSSYAMAWFRQAPGKEREFVAVISLSGGSVDYRDSVKGRFTISRDNAQSTVYLQMNSLRPEDTALYYCAARPVYMKWSERDFDYWGQGTLVTVSS

SEQ ID NO: 732 - protein, 123 aa, synthetic construct
EVQLVESGGGVVQPGGSLRLSCAASGFTFSSYAMAWFRQAPGKEREFVAVISLSGGSVDYRDSVKGRFTISRDNAQSTVSLQMNSLRPEDTALYYCAARPVYIKWSERDFDYWGQGTLVTVSS

SEQ ID NO: 733 - protein, 123 aa, synthetic construct
EVQLVESGGGVVQPGGSLRLSCAASGFTFSSYAMAWFRQAPGKEREFVAVISLSGGSVDYRDSVKGRFTISRDNAQSTVSLQMNSLRPEDTALYYCAARPVYLKWSERDFDYWGQGTLVTVSS

SEQ ID NO: 734 - protein, 123 aa, synthetic construct
EVQLVESGGGVVQPGGSLRLSCAASGFTFSSYAMAWFRQAPGKEREFVAVISLSGGSVDYRDSVKGRFTISRDNAQSTVSLQMNSLRPEDTALYYCAARPVYVKWSERDFDYWGQGTLVTVSS

SEQ ID NO: 735 - protein, 123 aa, synthetic construct
EVQLVESGGGVVQPGGSLRLSCAASGFTFSSYAMAWFRQAPGKEREFVAVISLSGGSVDYRDSVKGRFTISRDNAQSTVSLQMNSLRPEDTALYYCAARPVYMKWSERDFDYWGQGTLVTVSS

SEQ ID NO: 736 - protein, 123 aa, synthetic construct
EVQLVESGGGVVQPGGSLRLSCAASGFTFSSYAMAWFRQAPGKEREFVAVISLSGGSVDYADSVKGRFTISRDNAKNTVSLQMNSLRPEDTALYYCAARPVYIKWSERDFDYWGQGTLVTVSS

SEQ ID NO: 737 - protein, 123 aa, synthetic construct
EVQLVESGGGVVQPGGSLRLSCAASGFTFSSYAMAWFRQAPGKEREFVAVISLSGGSVDYRDSVKGRFTISRDNAKNTVSLQMNSLRPEDTALYYCARRPVYIKWSERDFDYWGQGTLVTVSS

SEQ ID NO: 738 - protein, 123 aa, synthetic construct
EVQLVESGGGVVQPGGSLRLSCAASGFTFSSYAMAWFRQAPGKEREFVAVISLSGGSVDYRDSVKGRFTISRDNAKNTVDLQMNSLRPEDTALYYCARRPVYIKWSERDFDYWGQGTLVTVSS

SEQ ID NO: 739 - protein, 123 aa, synthetic construct
EVQLVESGGGVVQPGGSLRLSCAASGFTFSSYAMAWFRQAPGKEREFVAVISLSGGSVDYRDSVKGRFTISRDNAKNTVSLQMNSLRPEDTALYYCAARPVYIKWSERDFDYWGQGTLVTVSS

SEQ ID NO: 740 - protein, 5 aa, synthetic construct
SYAMA

SEQ ID NO: 741 - protein, 17 aa, synthetic construct
VISLSGGSVDYRDSVKG

SEQ ID NO: 742 - protein, 17 aa, synthetic construct
VISLSGGSVDYADSVKG

SEQ ID NO: 743 - protein, 14 aa, synthetic construct
RPVYMKWSERDFDY

SEQ ID NO: 744 - protein, 14 aa, synthetic construct
RPVYIKWSERDFDY

SEQ ID NO: 745 - protein, 14 aa, synthetic construct
RPVYLKWSERDFDY

SEQ ID NO: 746 - protein, 14 aa, synthetic construct
RPVYVKWSERDFDY

SEQ ID NO: 747 - protein, 10 aa, synthetic construct
GFTFSSYAMA

SEQ ID NO: 748 - protein, 10 aa, synthetic construct
VISLSGGSVD

SEQ ID NO: 749 - protein, 14 aa, synthetic construct
RPVYMKWSERDFDY

SEQ ID NO: 750 - protein, 14 aa, synthetic construct
RPVYIKWSERDFDY

SEQ ID NO: 751 - protein, 14 aa, synthetic construct
RPVYLKWSERDFDY

SEQ ID NO: 752 - protein, 14 aa, synthetic construct
RPVYVKWSERDFDY

SEQ ID NO: 753 - protein, 5 aa, synthetic construct
DRSMA

SEQ ID NO: 754 - protein, 16 aa, synthetic construct
VNTWNGYTYTDESVEG

SEQ ID NO: 755 - protein, 15 aa, synthetic construct
SSYTYNIRQRSSYDW

SEQ ID NO: 756 - protein, 10 aa, synthetic construct
GRTIGDRSMA

SEQ ID NO: 757 - protein, 9 aa, synthetic construct
VNTWNGYTY

SEQ ID NO: 758 - protein, 15 aa, synthetic construct
SSYTYNIRQRSSYDW

SEQ ID NO: 759 - protein, 5 aa, synthetic construct
GYSVG

SEQ ID NO: 760 - protein, 17 aa, synthetic construct
VITRGGDYRYFAESVQG

SEQ ID NO: 761 - protein, 15 aa, synthetic construct
GTMTYAIRETRAYNY

SEQ ID NO: 762 - protein, 10 aa, synthetic construct
GLTFAGYSVG

SEQ ID NO: 763 - protein, 10 aa, synthetic construct
VITRGGDYRY

SEQ ID NO: 764 - protein, 15 aa, synthetic construct
GTMTYAIRETRAYNY

SEQ ID NO: 765 - protein, 5 aa, synthetic construct
SYTTG

SEQ ID NO: 766 - protein, 17 aa, synthetic construct
AITKNGPYLYYKDSVRG

SEQ ID NO: 767 - protein, 15 aa, synthetic construct
SDAQYDIRNREKYYY

SEQ ID NO: 768 - protein, 10 aa, synthetic construct
GRTLSSYTTG

SEQ ID NO: 769 - protein, 10 aa, synthetic construct
AITKNGPYLY

SEQ ID NO: 770 - protein, 15 aa, synthetic construct
SDAQYDIRNREKYYY

SEQ ID NO: 771 - protein, 5 aa, synthetic construct
NYAMG

SEQ ID NO: 772 - protein, 17 aa, synthetic construct
TISAAGHRTIFTNSVRG

SEQ ID NO: 773 - protein, 15 aa, synthetic construct
SPSQYGSMGKSAYDY

SEQ ID NO: 774 - protein, 10 aa, synthetic construct
GLTFDNYAMG

SEQ ID NO: 775 - protein, 10 aa, synthetic construct
TISAAGHRTI

SEQ ID NO: 776 - protein, 15 aa, synthetic construct
SPSQYGSMGKSAYDY

SEQ ID NO: 777 - protein, 5 aa, synthetic construct
SYVAA

SEQ ID NO: 778 - protein, 17 aa, synthetic construct
AASLSGGTTDYADSVKG

SEQ ID NO: 779 - protein, 14 aa, synthetic construct
RPVYLKYTYHDYDF

SEQ ID NO: 780 - protein, 10 aa, synthetic construct
GRTFSSYVAA

SEQ ID NO: 781 - protein, 10 aa, synthetic construct
AASLSGGTTD

SEQ ID NO: 782 - protein, 14 aa, synthetic construct
RPVYLKYTYHDYDF

SEQ ID NO: 783 - protein, 5 aa, synthetic construct
FTTMG

SEQ ID NO: 784 - protein, 16 aa, synthetic construct
YITESGSTAYTASVKD

SEQ ID NO: 785 - protein, 10 aa, synthetic construct
VIMMGGSGTY

SEQ ID NO: 786 - protein, 10 aa, synthetic construct
GSRLSFTTMG

SEQ ID NO: 787 - protein, 9 aa, synthetic construct
YITESGSTA

SEQ ID NO: 788 - protein, 10 aa, synthetic construct
VIMMGGSGTY

SEQ ID NO: 789 - protein, 5 aa, synthetic construct
SYAMA

SEQ ID NO: 790 - protein, 32 aa, synthetic construct; identical to SEQ ID NO: 282

SEQ ID NO: 791 - protein, 32 aa, synthetic construct; identical to SEQ ID NO: 303

SEQ ID NO: 792 - protein, 25 aa, synthetic construct
EVQLVESGGGLVQAGGSLRLSCAAS

SEQ ID NO: 793 - protein, 39 aa, synthetic construct
YRDSVKGRFTISRDNAQSTVSLQMNNLKPEDTAIYYCAA

SEQ ID NO: 794 - protein, 39 aa, synthetic construct
YRDSVKGRFTISRDNAKNTVDLQMNSLRPEDTALYYCAA

SEQ ID NO: 795 - protein, 39 aa, synthetic construct
YRDSVKGRFTISRDNAKNTVSLQMNSLRPEDTALYYCAA

SEQ ID NO: 796 - protein, 32 aa, synthetic construct
RFTISRDNAQSTVSLQMNNLRPEDTALYYCAA

SEQ ID NO: 797 - protein, 32 aa, synthetic construct
RFTISRDNAQSTVSLQMNSLRPEDTALYYCAA

SEQ ID NO: 798 - protein, 32 aa, synthetic construct
RFTISRDNAKSTVSLQMNSLRPEDTALYYCAA

SEQ ID NO: 799 - protein, 32 aa, synthetic construct; identical to SEQ ID NO: 798

SEQ ID NO: 800 - protein, 32 aa, synthetic construct; identical to SEQ ID NO: 798

SEQ ID NO: 801 - protein, 32 aa, synthetic construct
RFTISRDNAQSTVYLQMNSLRPEDTALYYCAA

SEQ ID NO: 802 - protein, 32 aa, synthetic construct
RFTISRDNAKNTVSLQMNSLRPEDTALYYCAR

SEQ ID NO: 803 - protein, 32 aa, synthetic construct
RFTISRDNAKNTVDLQMNSLRPEDTALYYCAR

SEQ ID NO: 804 - protein, 39 aa, synthetic construct
YRDSVKGRFTISRDNAQSTVSLQMNNLRPEDTALYYCAA

SEQ ID NO: 805 - protein, 39 aa, synthetic construct
YADSVKGRFTISRDNAQSTVSLQMNSLRPEDTALYYCAA

SEQ ID NO: 806 - protein, 39 aa, synthetic construct
YRDSVKGRFTISRDNAKSTVSLQMNSLRPEDTALYYCAA

SEQ ID NO: 807 - protein, 39 aa, synthetic construct
YADSVKGRFTISRDNAKSTVSLQMNSLRPEDTALYYCAA

SEQ ID NO: 808 - protein, 39 aa, synthetic construct
YRDSVKGRFTISRDNAQNTVSLQMNSLRPEDTALYYCAA

SEQ ID NO: 809 - protein, 39 aa, synthetic construct
YADSVKGRFTISRDNAQNTVSLQMNSLRPEDTALYYCAA

SEQ ID NO: 810 - protein, 39 aa, synthetic construct; identical to SEQ ID NO: 233

SEQ ID NO: 811 - protein, 39 aa, synthetic construct
YRDSVKGRFTISRDNAQSTVYLQMNSLRPEDTALYYCAA

SEQ ID NO: 812 - protein, 39 aa, synthetic construct
YRDSVKGRFTISRDNAQSTVSLQMNSLRPEDTALYYCAA

SEQ ID NO: 813 - protein, 39 aa, synthetic construct
YRDSVKGRFTISRDNAKNTVSLQMNSLRPEDTALYYCAR

SEQ ID NO: 814 - protein, 39 aa, synthetic construct
YRDSVKGRFTISRDNAKNTVDLQMNSLRPEDTALYYCAR

SEQ ID NO: 815 - protein, 17 aa, synthetic construct
VSSLSGGTSDYADSVKG

SEQ ID NO: 816 - protein, 14 aa, synthetic construct
RPVYIRYSIRDYDI

SEQ ID NO: 817 - protein, 10 aa, synthetic construct
GRTMSSYAMA

SEQ ID NO: 818 - protein, 10 aa, synthetic construct
VSSLSGGTSD

SEQ ID NO: 819 - protein, 14 aa, synthetic construct
RPVYIRYSIRDYDI

SEQ ID NO: 820 - protein, 30 aa, synthetic construct
EVQLVESGGGLVQAGGSLRLSCAASGFTFS

SEQ ID NO: 821 - protein, 14 aa, synthetic construct
WFRQAPGKEREFVA

SEQ ID NO: 822 - protein, 14 aa, synthetic construct
WFRQAPGKEREFVA

SEQ ID NO: 823 - protein, 14 aa, synthetic construct
WFRQAPGKEREFVA

SEQ ID NO: 824 - protein, 14 aa, synthetic construct
WFRQAPGKEREFVA

SEQ ID NO: 825 - protein, 14 aa, synthetic construct
WFRQAPGKEREFVA

SEQ ID NO: 826 - protein, 14 aa, synthetic construct
WFRQAPGKEREFVA

SEQ ID NO: 827 - protein, 32 aa, synthetic construct
RFTISRDNAQSTVSLQMNNLKPEDTAIYYCAA

SEQ ID NO: 828 - protein, 11 aa, synthetic construct
WGQGTLVTVSS

SEQ ID NO: 829 - protein, 11 aa, synthetic construct
WGQGTLVTVSS

SEQ ID NO: 830 - protein, 11 aa, synthetic construct
WGQGTLVTVSS

SEQ ID NO: 831 - protein, 11 aa, synthetic construct
WGQGTLVTVSS

SEQ ID NO: 832 - protein, 11 aa, synthetic construct
WGQGTLVTVSS

SEQ ID NO: 833 - protein, 11 aa, synthetic construct
WGQGTLVTVSS

SEQ ID NO: 834 - protein, 30 aa, synthetic construct
EVQLVESGGGVVQPGGSLRLSCAASGFTFS

SEQ ID NO: 835 - protein, 30 aa, synthetic construct; identical to SEQ ID NO: 834

SEQ ID NO: 836 - protein, 25 aa, synthetic construct
EVQLVESGGGVVQPGGSLRLSCAAS

SEQ ID NO: 837 - protein, 25 aa, synthetic construct
EVQLVESGGGVVQPGGSLRLSCAAS

SEQ ID NO: 838 - protein, 14 aa, synthetic construct
  VARIANT 5: note = X = M, I, L, or V
RPVYXKWSERDFDY

SEQ ID NO: 839 - protein, 17 aa, synthetic construct
  VARIANT 12: note = X = R or A
VISLSGGSVDYXDSVKG

SEQ ID NO: 840 - protein, 20 aa, synthetic construct
METDTLLLWVLLLWVPGSTG

SEQ ID NO: 841 - protein, 5 aa, synthetic construct
SYAMG

SEQ ID NO: 842 - protein, 17 aa, synthetic construct
TISLSGGTTWYADSVKG

SEQ ID NO: 843 - protein, 14 aa, synthetic construct
RPVYHKTSARDYDF

SEQ ID NO: 844 - protein, 5 aa, synthetic construct
TYGMG

SEQ ID NO: 845 - protein, 17 aa, synthetic construct
VISSDGVRTNYADSVQG

SEQ ID NO: 846 - protein, 13 aa, synthetic construct
GSGLFSAGYEYQY

SEQ ID NO: 847 - protein, 5 aa, synthetic construct
INGMG

SEQ ID NO: 848 - protein, 17 aa, synthetic construct
ATRWNGGTKYYTSSVEG

SEQ ID NO: 849 - protein, 15 aa, synthetic construct
TRRRFDFYSVGGYEN

SEQ ID NO: 850 - protein, 5 aa, synthetic construct
TKAMG

SEQ ID NO: 851 - protein, 17 aa, synthetic construct
AITWTAGLTYLADSAKG

SEQ ID NO: 852 - protein, 19 aa, synthetic construct
RRTFSYEHPNYYTASAYHF

SEQ ID NO: 853 - protein, 5 aa, synthetic construct
DYTLA

SEQ ID NO: 854 - protein, 16 aa, synthetic construct
VSTWNGYTYYGESVSG

SEQ ID NO: 855 - protein, 17 aa, synthetic construct
VISLSGQNTYYADSVKG

SEQ ID NO: 856 - protein, 14 aa, synthetic construct
RPVYHKFHYRDYDF

SEQ ID NO: 857 - protein, 10 aa, synthetic construct
GDTFGTKAMG

SEQ ID NO: 858 - protein, 10 aa, synthetic construct
AITWTAGLTY

SEQ ID NO: 859 - protein, 19 aa, synthetic construct
RRTFSYEHPNYYTASAYHF